(19) United States
(12) Patent Application Publication
(10) Pub. No.: US 2025/0361239 A1
(43) Pub. Date: Nov. 27, 2025

(54) PROTEIN DEGRADERS AND USES THEREOF

(71) Applicant: ZHUHAI YUFAN BIOTECHNOLOGIES CO., LTD., Zhuhai, Guangdong (CN)

(72) Inventors: Xingyu LIN, Zhuhai, Guangdong (CN); Na AN, Zhuhai, Guangdong (CN); Tingting LU, Zhuhai, Guangdong (CN)

(21) Appl. No.: 18/873,159

(22) PCT Filed: Jun. 9, 2023
(86) PCT No.: PCT/CN2023/099547

§ 371 Date: Dec. 9, 2024

(30) Foreign Application Priority Data
Jun. 10, 2022 (CN) 202210654080.7

(51) Int. Cl. C07D 487/14; A61K 31/454; A61K 31/4545; A61K 31/496; A61K 31/4985; A61K 31/506; A61K 31/52; A61P 35/00; A61P 35/02; C07D 401/04; C07D 401/14; C07D 403/14; C07D 409/14; C07D 413/14; C07D 417/14; C07D 471/04; C07D 471/14; C07D 473/00; C07D 495/04; C07D 498/04; C07D 513/04

(52) U.S. Cl. CPC C07D 487/14; A61K 31/454; A61K 31/4545; A61K 31/496; A61K 31/4985; A61K 31/506; A61K 31/52; A61P 35/00; A61P 35/02; C07D 401/04; C07D 401/14; C07D 403/14; C07D 409/14; C07D 413/14; C07D 417/14; C07D 471/04; C07D 471/14; C07D 473/00; C07D 495/04; C07D 498/04; C07D 513/04

(57) ABSTRACT

The present invention discloses a protein degrader such as a GSPT1 degrader and use thereof, particularly use in the prevention and/or treatment of diseases.

This application claims priority to Chinese Patent Application No. CN202210654080.7, filed Jun. 10, 2022, the entire contents of which are herein incorporated by reference for all purposes.

### TECHNICAL FIELD

The present invention relates to the technical field of biomedicine, and particularly to protein degraders such as a GSPT1 degrader and use thereof.

### BACKGROUND

Protein dysfunction and/or protein imbalance are signs of many disease states. For example, the function of the immune system is well balanced through the activity of pro- and anti-inflammatory mediators or cytokines. Protein synthesis disorder may lead to uncontrolled cell growth, proliferation and migration and thus to cancer. For example, the translation termination factor GSPT1 (eRF3a) mediates stop codon recognition, facilitating ribosome's release of nascent peptides. In addition to its role in translation termination, GSPT1 is also involved in several other key cellular processes, such as cell cycle regulation, cytoskeletal organization, and apoptosis. GSPT1 is considered an oncogenic driver for several different types of cancer, including breast cancer, hepatocellular cancer, gastric cancer, and prostate cancer. GSPT1 is also involved in glial scar formation and astrocyte proliferation following injuries to the central nervous system.

One way to destroy disease protein drivers is to reduce the cellular concentration of these proteins. For example, proteolytic degradation of cellular proteins is of critical importance to normal cellular functions. The process of intercepting specific disease-related proteins provides a new mechanism for treating the disease. The irreversibility of proteolysis makes them very suitable as regulatory switches for controlling unidirectional processes.

At present, there are not many reports about GSPT1-targeting degraders. The development of new GSPT1 degrader compounds is beneficial to the variety of candidates and drugs for the treatment of GSPT1-associated diseases.

### SUMMARY

In various embodiments, the present invention generally relates to protein degraders, such as a GSPT1 degrader. Without wishing to be bound by theories, it is believed that compounds of the present disclosure can act as a molecular glue, which is capable of binding to an E3 ligase, which can lead to the recruitment, ubiquitination, and then degradation of various substrates that are not normally substrates of the ligase. Without wishing to be bound by theories, it is also believed that the compounds of the present disclosure can be viewed as containing an E3 ligase binding portion, typically a glutarimide containing moiety or other moiety that can bind to a cereblon pocket, which is linked to a structure that can bind to the protein substrate.

In some embodiments, the present invention provides a compound that can be used as a GSPT1 degrader, and a pharmaceutically acceptable salt, a stereoisomer, an ester, a prodrug, a solvate and a deuterated compound thereof, as well as a preparation method therefor and use thereof, particularly use thereof in preventing and/or treating diseases. In some embodiments, the compounds herein can also used as a degrader for proteins other than GSPT1.

In various embodiments, the compounds of the present disclosure can be characterized as having a structure according to Formula (I), Formula A, Formula B, Formula C, or a subformula thereof, as defined herein.

In a first aspect, the present invention provides a compound having the following structure:

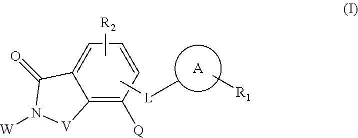

wherein,
W is selected from: C₀₋₁₀ alkyl, C₃₋₆ cycloalkyl, heterocycloalkyl, and heteroaryl;
R₁ is one or more independent substituents of ring A and is selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), and

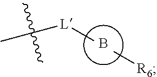

L′ is selected from: single bond, C₁₋₁₀ alkylene, —O—, —O—C₁₋₁₀ alkylene-, —S—, —S—C₁₋₁₀ alkylene-, —SO—, —SO—C₁₋₁₀ alkylene-, —SO₂—, —SO₂—C₁₋₁₀ alkylene-, —N(C₀₋₁₀ alkyl)-, —N—(C₀₋₁₀ alkyl) alkylene-, —CO—, —CO—C₁₋₁₀ alkylene-, —CONH—, and —CONH—C₁₋₁₀ alkylene-; R₆ is one or more independent substituents on ring B and is selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl; R₂ is one or more independent substituents of a benzene ring and is selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, aryl, heterocycloalkyl, and heteroaryl;
V is selected from: O, S,

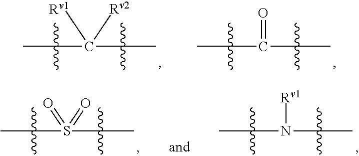

wherein R^{v1}, R^{v2} is independently selected from: C₀₋₁₀ alkyl, C₃₋₆ cycloalkyl, and heterocycloalkyl;
Q is H or a single bond (when Q is a single bond, the site in the benzene ring to which Q is linked is linked to R₂, or to L), or Q and V, together with carbon atoms therebetween, form aryl or heteroaryl;
L has the following structure:

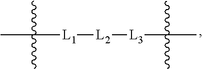

wherein,
L₁ is selected from: —NR^{L1}—(C₀₋₁₀ alkylene)-, —O—(C₀₋₁₀ alkylene)-, —S—(C₀₋₁₀ alkylene)-, —NR^{L1}CO—(C₀₋₁₀ alkylene)-, —CONR^{L1}—(C₀₋₁₀ alkylene)-, —CO—(C₀₋₁₀ alkylene)-, —NR^{L1}CONH—(C₀₋₁₀ alkylene)-, —(C₁₋₁₀ alkylene)-, —SO₂—(C₀₋₁₀ alkylene)-, —SO—(C₀₋₁₀ alkylene)-,

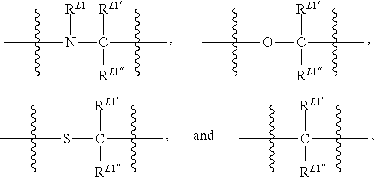

wherein R is selected from: H, —OH, C₁₋₆ alkyl, C₃₋₆ cycloalkyl, and heterocycloalkyl, wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, C₃₋₆ cycloalkyl, and heterocycloalkyl; R^{L1} and R^{L2} are independently selected from: halogen, C₁₋₆ alkyl, C₃₋₆ cycloalkyl, and heterocycloalkyl, wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, C₃₋₆ cycloalkyl, and heterocycloalkyl; or R^{L1′} and R^{L1″}, together with carbon atoms linked thereto, form cycloalkyl or heterocyclyl;
L₃ is selected from: —(C₀₋₁₀ alkylene)-, —(C₀₋₁₀ alkylene)-NR^{L3}—, —(C₀₋₁₀ alkylene)-O—, —(C₀₋₁₀ alkylene)-S—, —(C₀₋₁₀ alkylene)-NR^{L3}CO—, —(C₀₋₁₀ alkylene)-CONR^{L3}—, —(C₀₋₁₀ alkylene)-CO—, —(C₀₋₁₀ alkylene)-NHCONR^{L3}—, —(C₀₋₁₀ alkylene)-SO₂—, —(C₀₋₁₀ alkylene)-SO—,

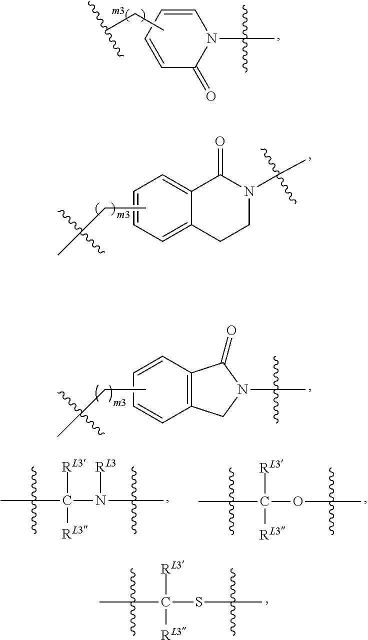

—NR^{L3}—(C₀₋₁₀ alkylene)-, —O—(C₀₋₁₀ alkylene)-, —S—(C₀₋₁₀ alkylene)-, —NR^{L3}CO—(C₀₋₁₀ alkylene)-, —CONR^{L3}—(C₀₋₁₀ alkylene)-, —CO—(C₀₋₁₀ alkylene)-, —NHCONR^{L3}—(C₀₋₁₀ alkylene)-, —SO₂—(C₀₋₁₀ alkylene)-, —SO—(C₀₋₁₀ alkylene)-,

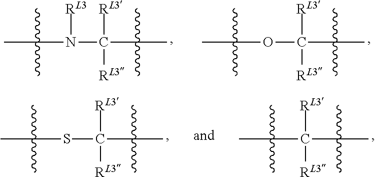

wherein R^{L3} is selected from: H, —OH, C₁₋₆ alkyl, C₃₋₆ cycloalkyl, and heterocycloalkyl, wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, C₃₋₆ cycloalkyl, and heterocycloalkyl; R^{L3′} and R^{L3″} are independently selected from: halogen, C₁₋₆ alkyl, C₃₋₆ cycloalkyl, and heterocycloalkyl, wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, C₃₋₆ cycloalkyl, and heterocycloalkyl; or R^{L3′} and R^{L3″}, together with carbon atoms linked thereto, form cycloalkyl or heterocyclyl; L₂ is a covalent bond, or a divalent, saturated or unsaturated, linear or branched C₁₋₅₀ (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₅, C₃₀, C₃₅, C₄₀, C₄₅, or C₅₀) hydrocarbon chain, wherein 0-6 methylene units are independently substituted with: -Cy-, —O—, —NR^{L2}—, —S—, —OC(O)—, —C(O)O—, —C(O)—, —S(O)—, —S(O)₂—, —NR^{L2}S(O)₂—, —S(O)₂—NR^{L2}—, —NR^{L2}—C(O)—, —C(O)NR^{L2}—, —OC(O)NR^{L2}—, —NR^{L2}—C(O)O—,

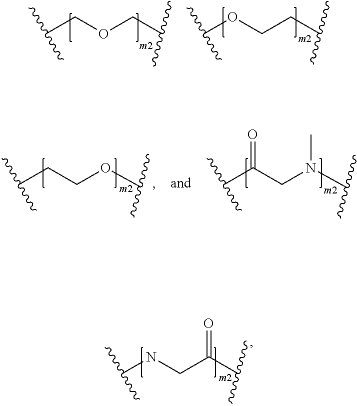

wherein m2 is an integer selected from 0-10 (e.g., 0, 1, 2, 3, 4, 5, 6, 7, 8, 9, or 10), and each -Cy- is independently selected from the following optionally substituted divalent rings: arylene, cycloalkylene, and heterocyclylene; R^{L2} is selected from: H, —OH, C₁₋₆ alkyl, C₃₋₆ cycloalkyl, and heterocycloalkyl, wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, C₃₋₆ cycloalkyl, and heterocycloalkyl.

In one embodiment of the present invention, Q is a single bond, and the compound described above may have the following structures:

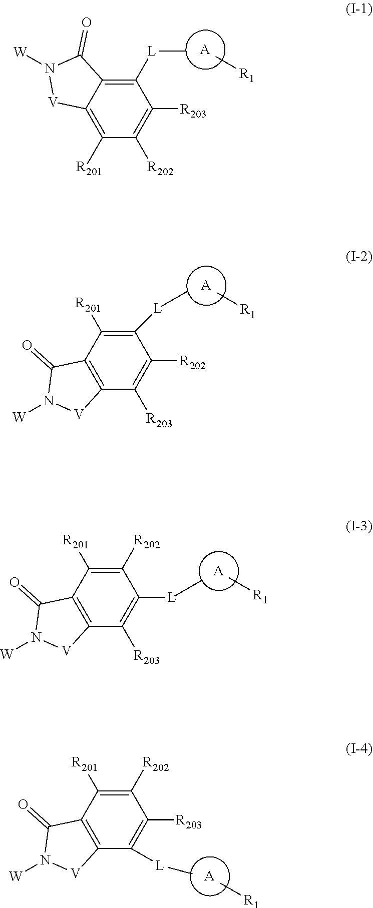

wherein R₂₀₁, R₂₀₂ and R₂₀₃ each have the definition as described above for R₂. In another embodiment of the present invention, Q and V, together with carbon atoms therebetween, form aryl or heteroaryl, particularly six-membered aryl or heteroaryl; for example, the compound described above may have the following structure:

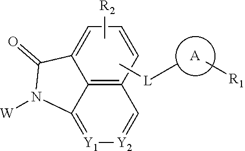

wherein Y₁ and Y₂ are independently selected from: CH and N.
Specifically, the Compound May have the Following Structures:

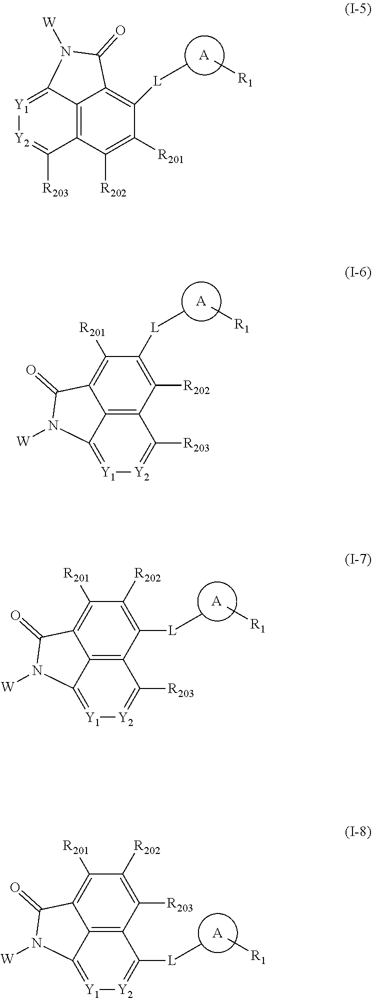

wherein R₂₀₁, R₂₀₂ and R₂₀₃ each have the definition as described above for R₂.

In some embodiments of the present invention, Y₁ and Y₂ are both CH.

In other embodiments of the present invention, Y₁ is CH, and Y₂ is N.

In other embodiments of the present invention, Y₁ is N, and Y₂ is CH.

In other embodiments of the present invention, Y₁ and Y₂ are both N.

In some embodiments of the present invention, R₂₀₁, R₂₀₂ and R₂₀₃ are independently selected from: cycloalkyl, aryl, heterocycloalkyl, and heteroaryl; in other embodiments of the present invention, R₂₀₁, R₂₀₂ and R₂₀₃ are independently selected from: halogen, —CN, —NO₂, —CF₃, —OCF₃, —H, C₁₋₆ alkyl (e.g., —CH₃, or —CH₂CH₃), —OH, —NH₂, and —N(C₁₋₃ alkyl)(C₁₋₃ alkyl); particularly, R₂₀₁, R₂₀₂ and R₂₀₃ are all —H.

In some embodiments of the present invention, W is

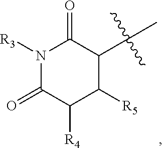

wherein R₃ is selected from: C₀₋₁₀ alkyl, C₃₋₆ cycloalkyl, heterocycloalkyl, and heteroaryl; R₄ and R₅ are independently selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl.

Specifically, V may be selected from: —CH₂—,

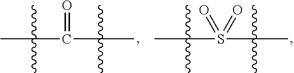

and —NH—; in some embodiments of the present invention, V is

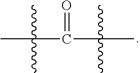 [SMILES: *C(*)=O]

Specifically, R₃ may be selected from: —H, and C₁₋₆ alkyl (e.g., —CH₃, or —CH₂CH₃); in some embodiments of the present invention, R₃ is —H.

Specifically, R₄ may be selected from: halogen, —CN, —NO₂, —CF₃, —OCF₃, —H, C₁₋₆ alkyl (e.g., —CH₃, or —CH₂CH₃), —OH, —NH₂, and —N(C₁₋₃ alkyl)(C₁₋₃ alkyl); in some embodiments of the present invention, R₄ is —H.

Specifically, R₅ may be selected from: halogen, —CN, —NO₂, —CF₃, —OCF₃, —H, C₁₋₆ alkyl (e.g., —CH₃, or —CH₂CH₃), —OH, —NH₂, and —N(C₁₋₃ alkyl)(C₁₋₃ alkyl); in some embodiments of the present invention, R₅ is —H.

Particularly, W is

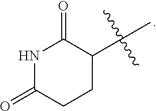 [SMILES: *C1CCC(=O)NC1=O]

Specifically, R₂ may be selected from: halogen, —CN, —NO₂, —CF₃, —OCF₃, —H, C₁₋₆ alkyl (e.g., —CH₃, or —CH₂CH₃), —OH, —NH₂, and —N(C₁₋₃ alkyl)(C₁₋₃ alkyl); in some embodiments of the present invention, R₂ is —H.

Further, the Compound Described Above May have the Following Structure:

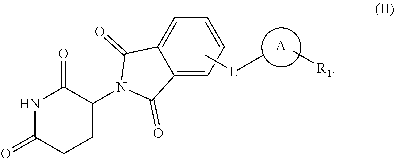

Still Further, the Compound Described Above May have the Following Structure:

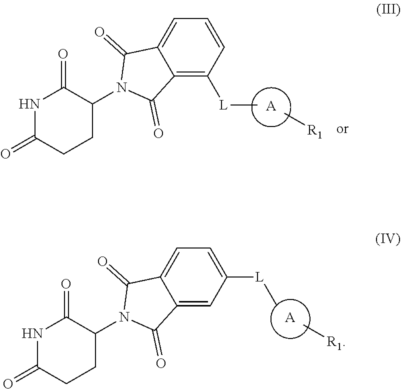

Further, the Compound Described Above May Also have the Following Structure:

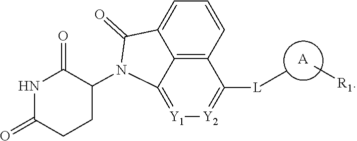

Still Further, the Compound Described Above May Also have the Following Structures:

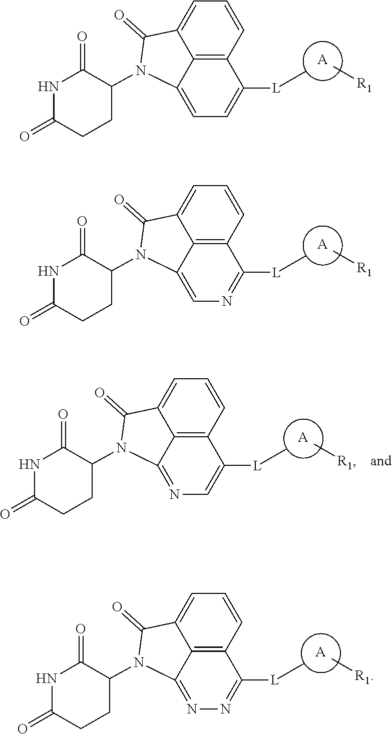

Specifically, each -Cy- is independently an optionally substituted divalent ring selected from: phenylene, bicyclic arylene, tricyclic arylene, monocyclic cycloalkylene, bicyclic cycloalkylene, tricyclic cycloalkylene, monocyclic heteroarylene, bicyclic heteroarylene, tricyclic heteroarylene, monocyclic heterocycloalkylene, bicyclic heterocycloalkylene, and tricyclic heterocycloalkylene; particularly, phenylene, 8- to 10-membered bicyclic arylene, 3- to 7-membered monocyclic saturated or partially unsaturated carbocyclylene, 4- to 7-membered saturated or partially unsaturated spirocarbocyclylene, 8- to 10-membered saturated or partially unsaturated fused carbocyclylene, 3- to 7-membered monocyclic saturated or partially unsaturated heterocyclylene, 4- to 7-membered saturated or partially unsaturated spiro heterocyclylene, and 8- to 10-membered saturated or partially unsaturated fused heterocyclylene.

More specifically, each -Cy- may be independently selected from:

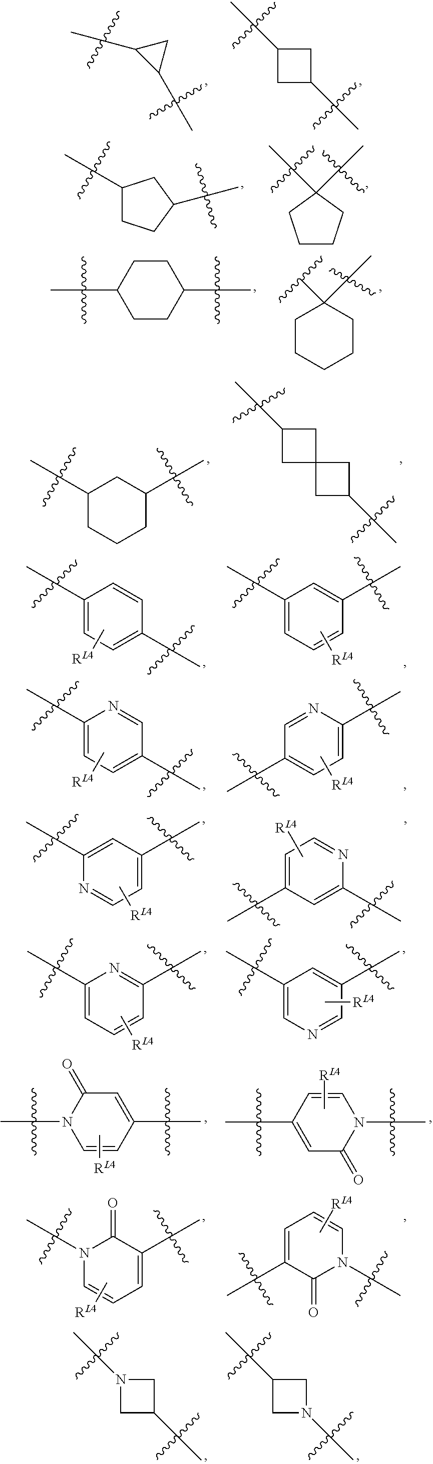

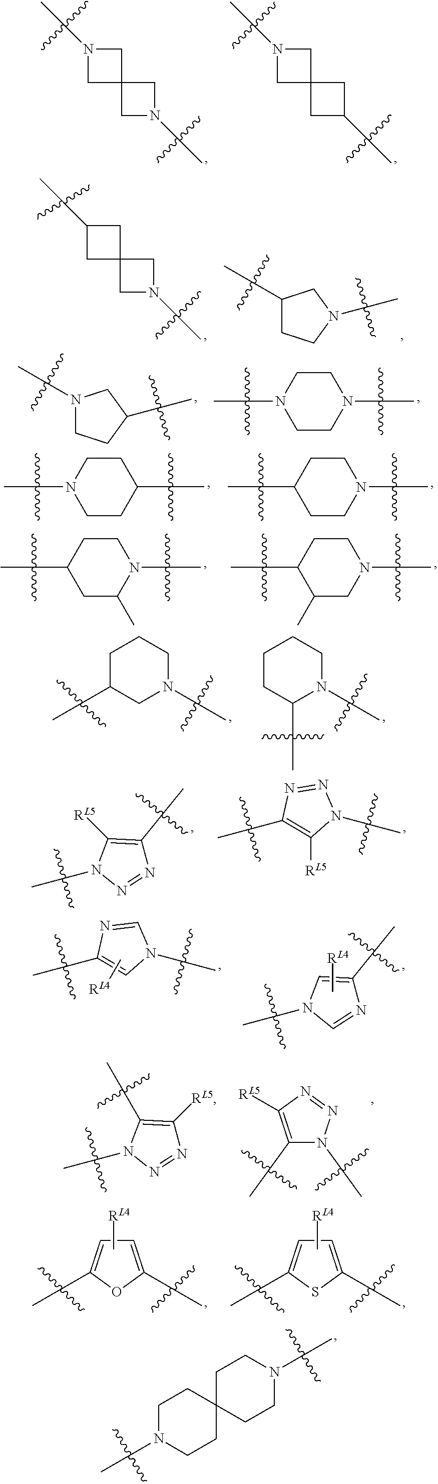

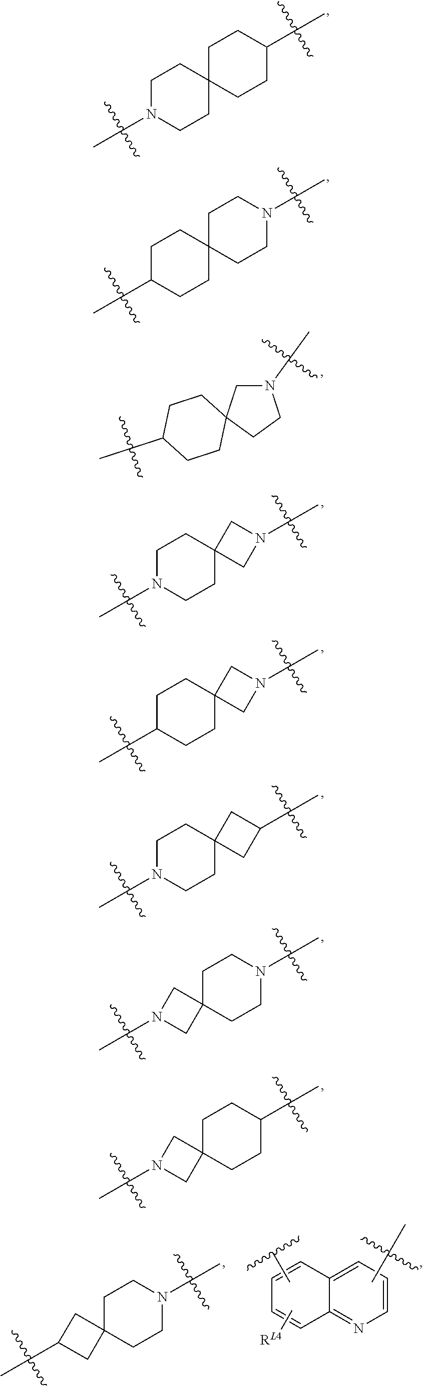

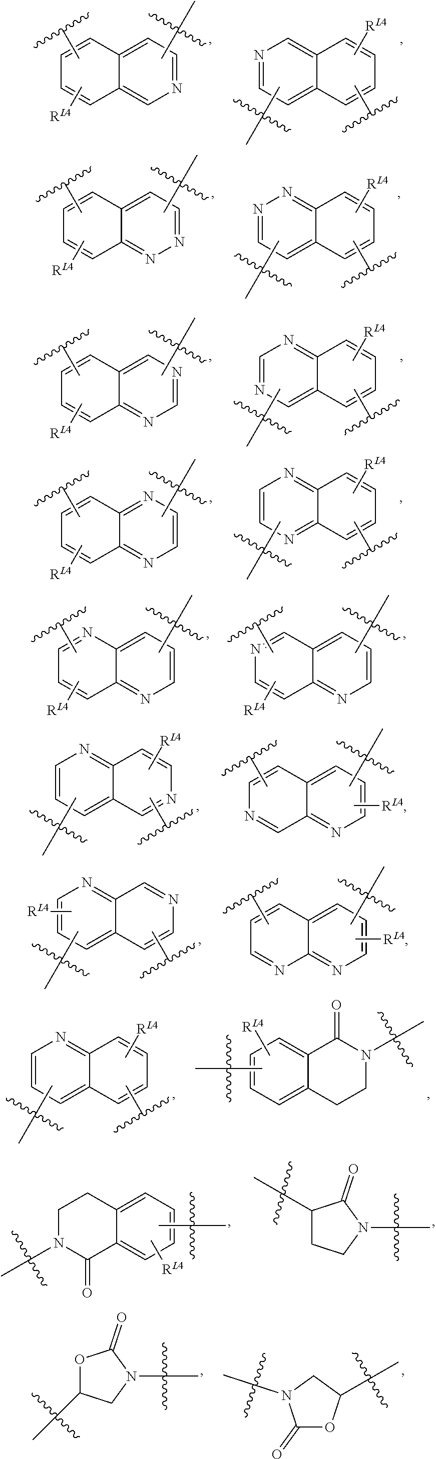

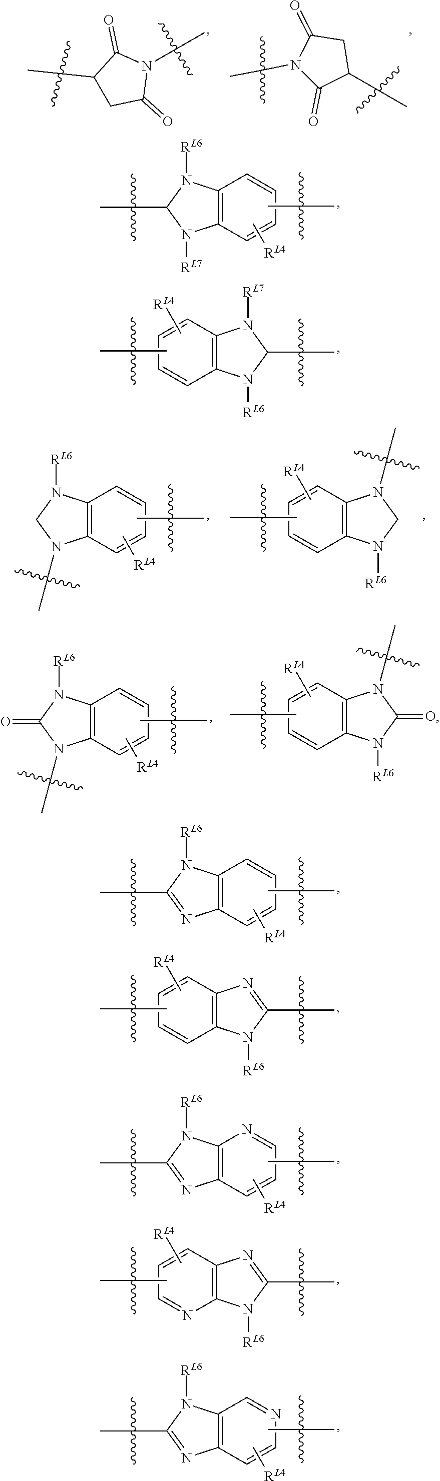

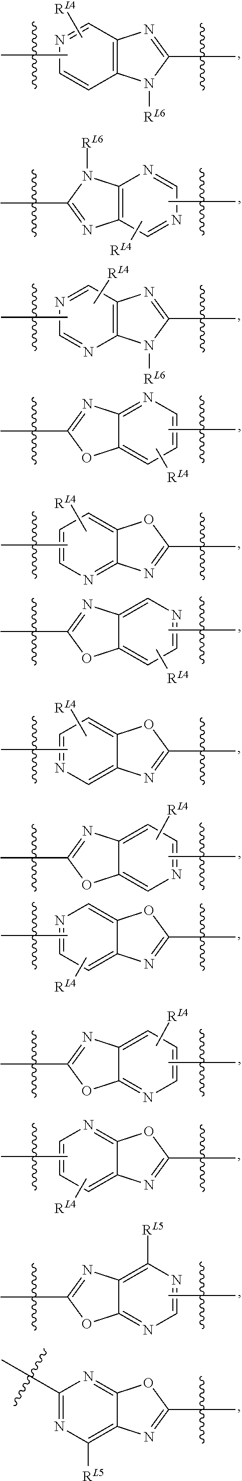

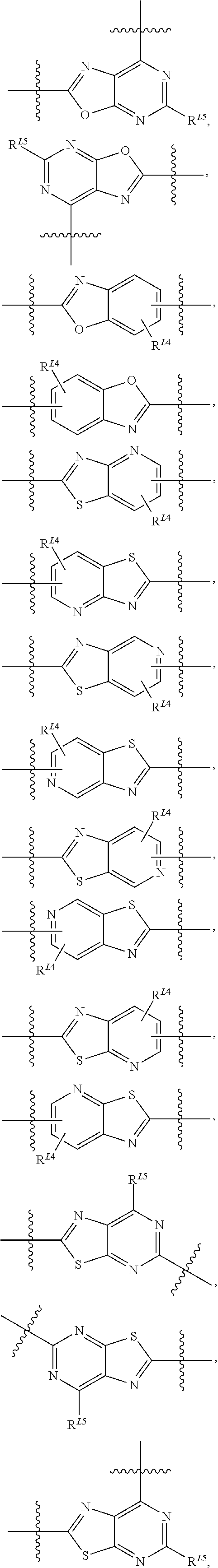

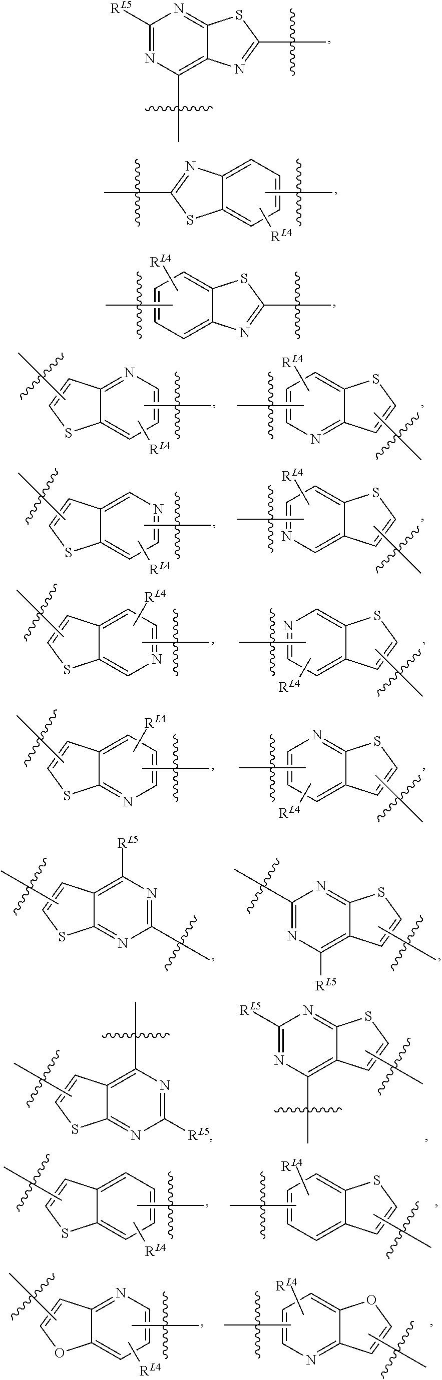

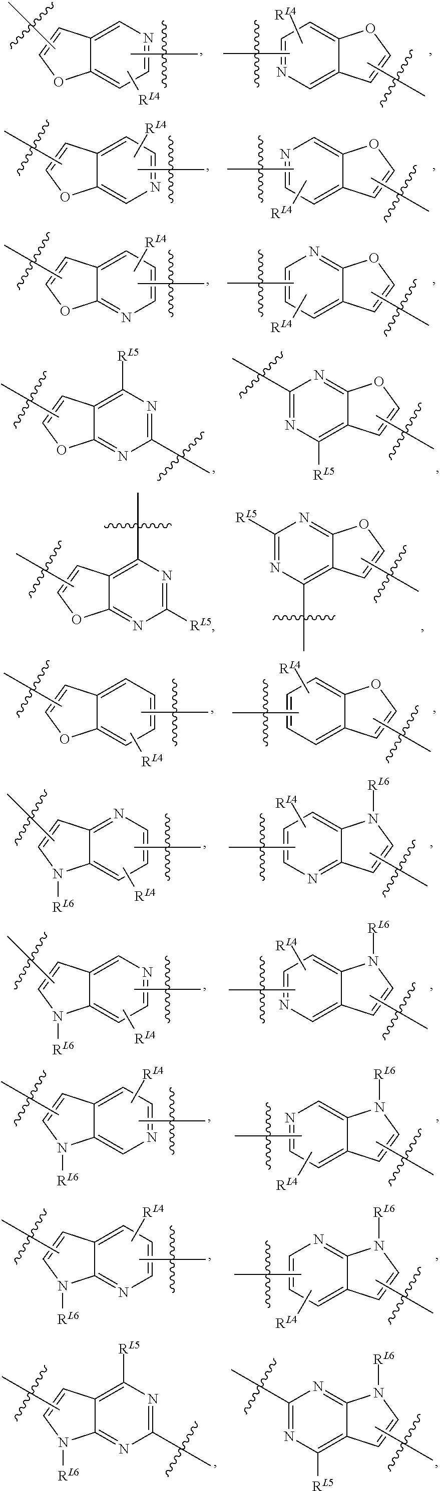

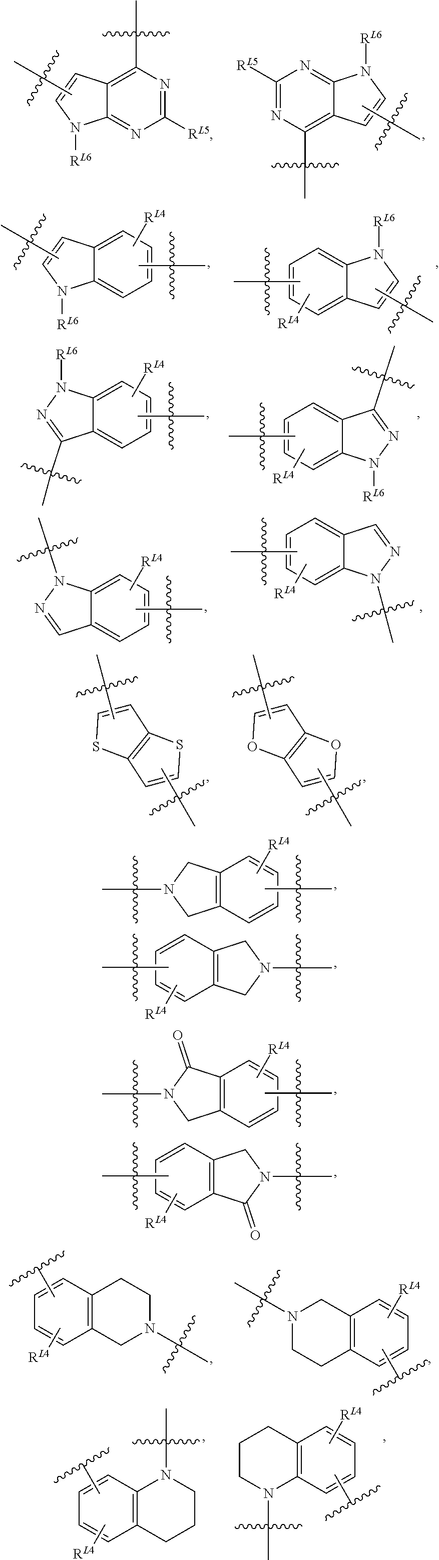

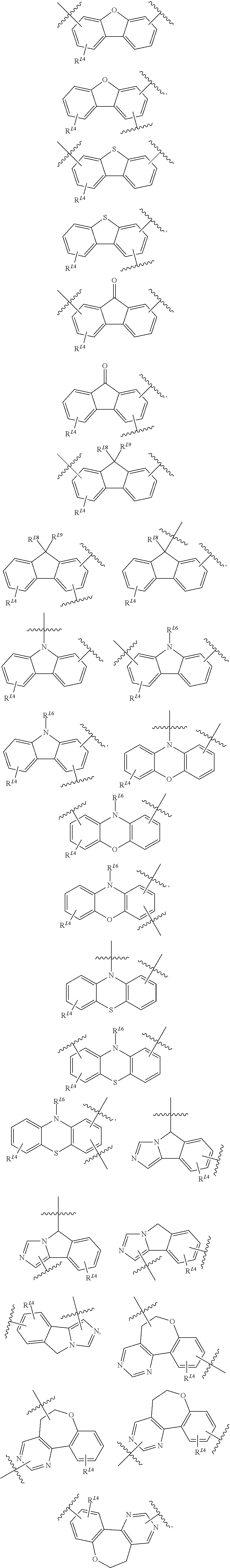

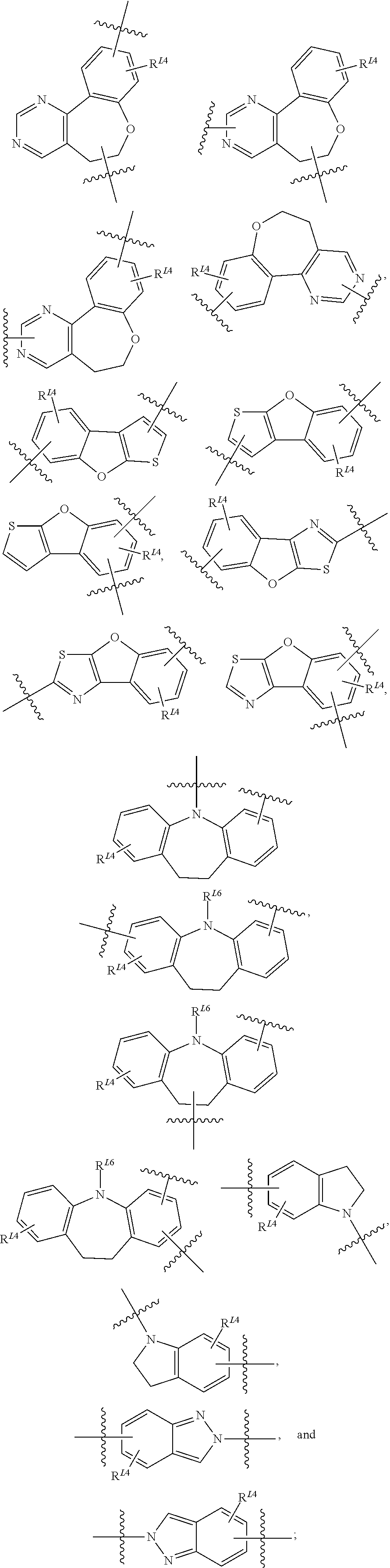

wherein R^{L4} is one or more independent substituents of a ring and is selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), and three- to six-membered heterocycloalkyl;
R^{L5} is selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), and —CO(C₀₋₁₀ alkyl);
R^{L6} and R^{L7} are independently selected from: C₀₋₁₀ alkyl, C₃₋₆ cycloalkyl, heterocycloalkyl, and heteroaryl;
R^{L8} and R^{L9} are independently selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), and —CO(C₀₋₁₀ alkyl); or R^{L8} and R^{L9}, together with a carbon atom linked to both R^{L8} and R^{L9}, form substituted or unsubstituted cycloalkyl or heterocyclyl.
   Further, Each -Cy- May be Independently Selected from:

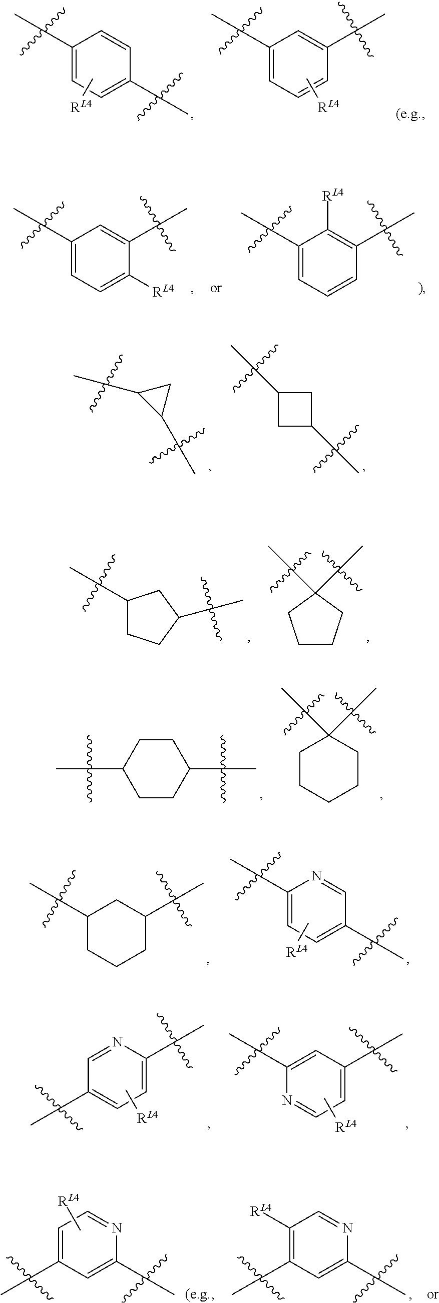

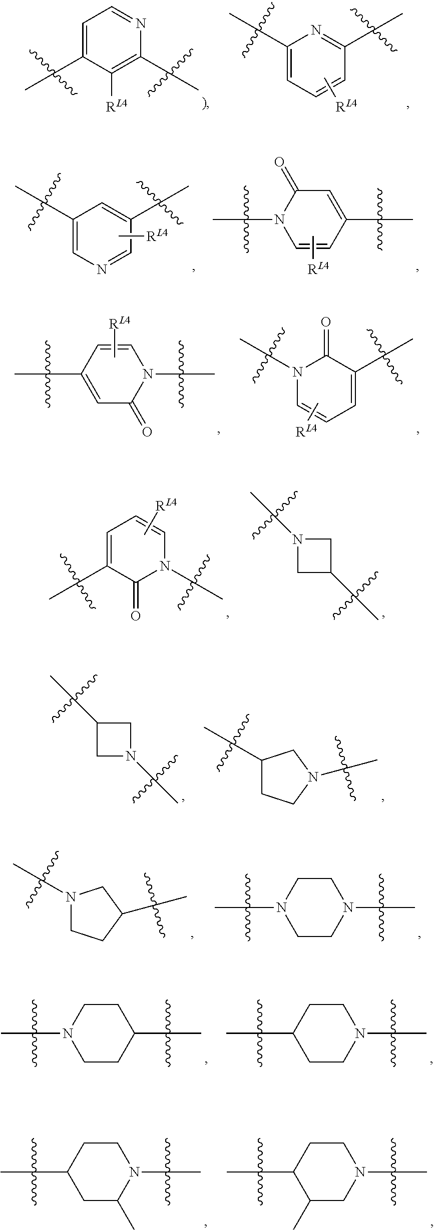

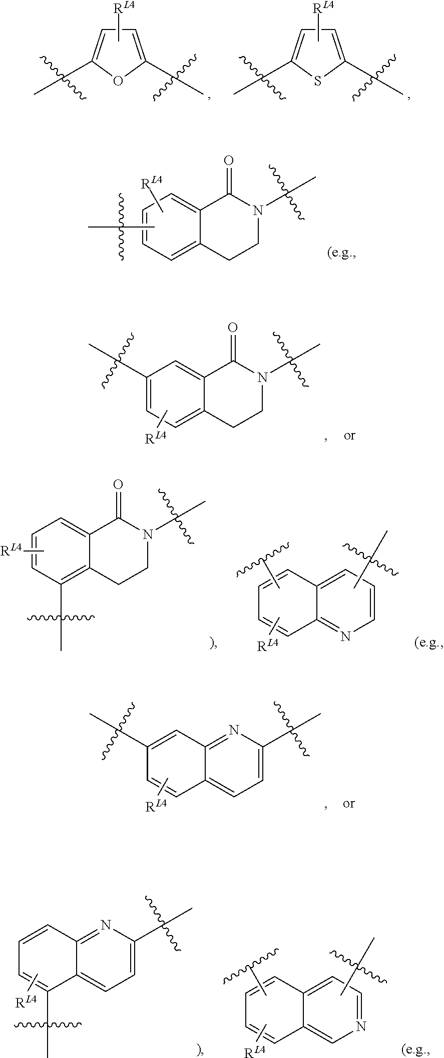

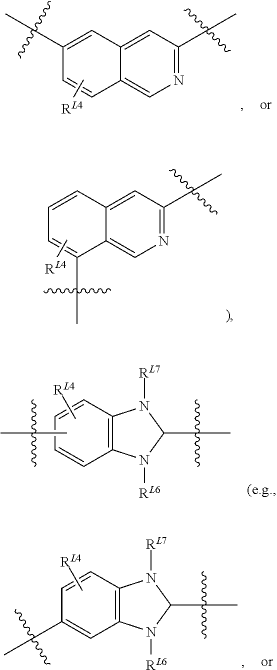

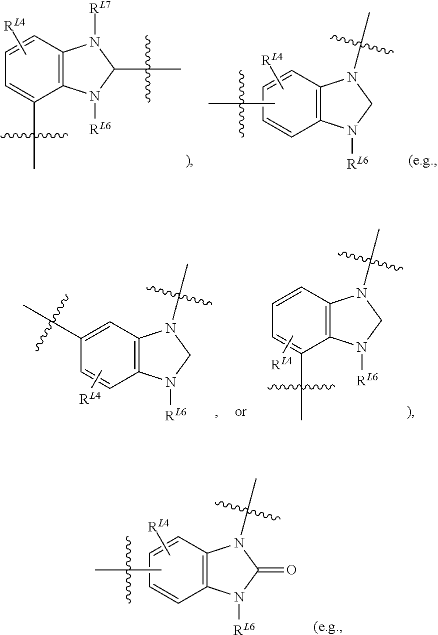

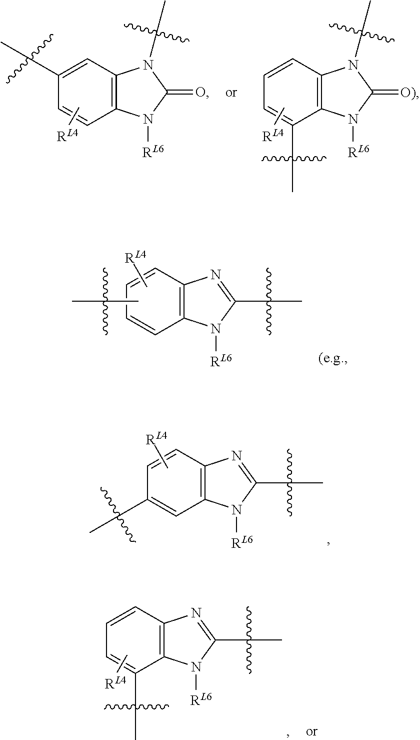

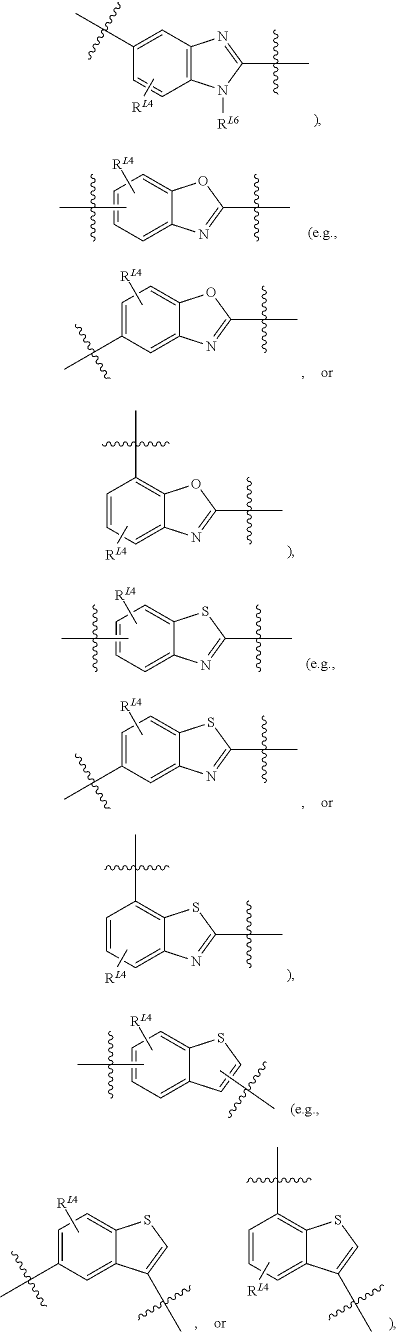

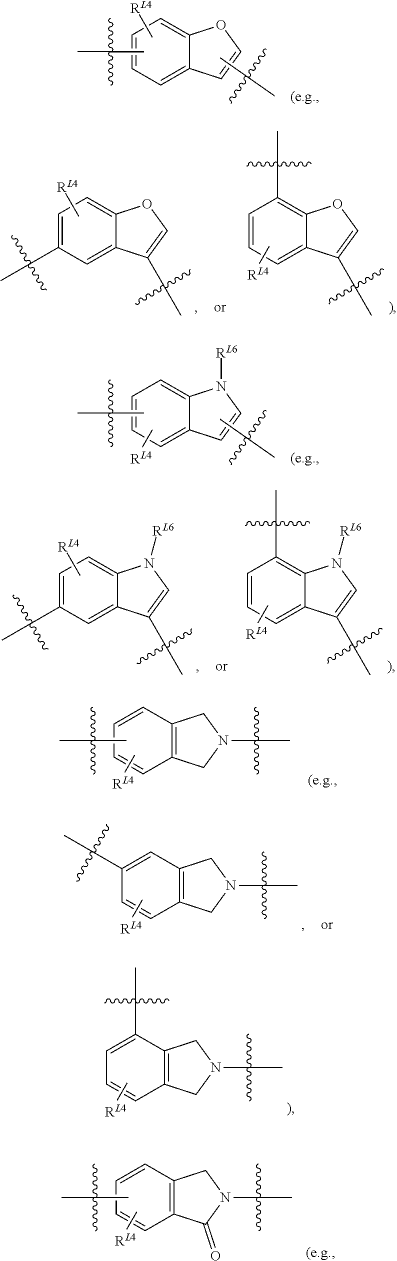

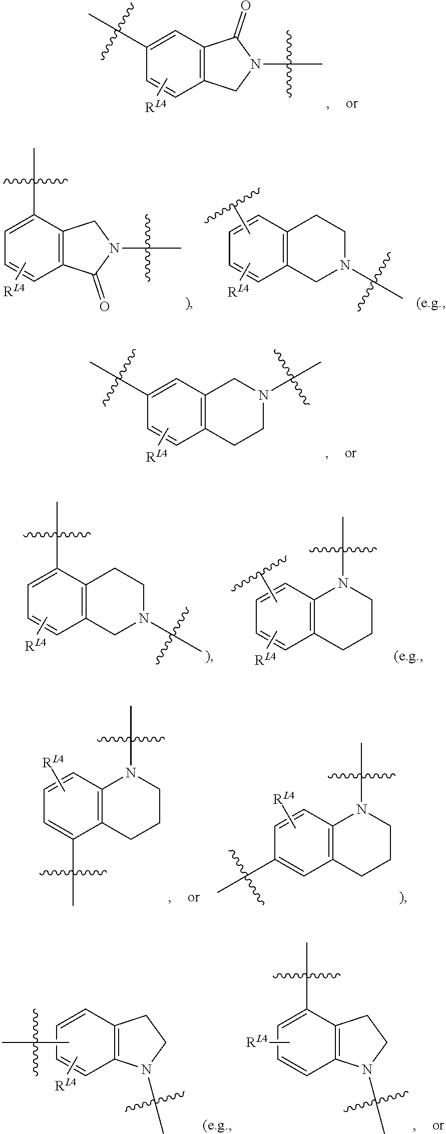

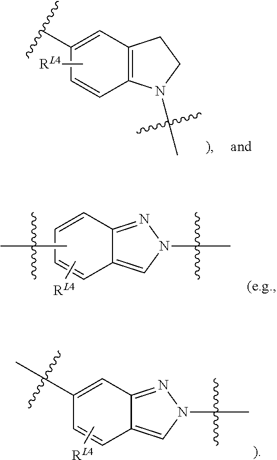

More specifically, R^{L4} may be selected from: —H, halogen (particularly F), —OH, C₁₋₆ alkyl (e.g., —CH₃, or —CH₂CH₃), —O(C₁₋₆ alkyl)(e.g., methoxy, or ethoxy), and four- to six-membered nitrogen-containing heterocycloalkyl; in some embodiments of the present invention, R^{L4} is selected from: —H, F, methoxy, ethoxy,

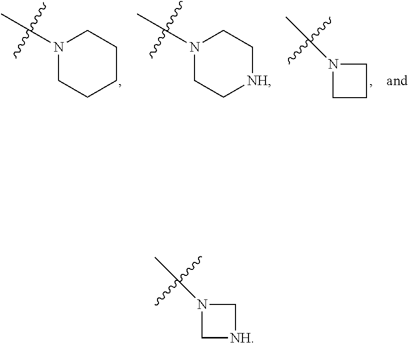 [SMILES: *N1CCCCC1 ; *N1CCNCC1 ; *N1CCC1 ; *N1CNC1]

More specifically, R^{L5} may be selected from: —H, halogen (particularly F), —OH, C₁₋₆ alkyl (e.g., —CH₃, or —CH₂CH₃), and —O(C₁₋₆ alkyl)(e.g., methoxy, or ethoxy); in some embodiments of the present invention, R^{L} is —H.

More specifically, R^{L6} may be selected from: —H, C₁₋₆ alkyl (e.g., —CH₃, or —CH₂CH₃), and C₃₋₆ cycloalkyl; in some embodiments of the present invention, R^{L6} is selected from: —H, —CH₃, —CH₂CH₃, cyclopropyl, and cyclobutyl.

In some embodiments of the present invention, each -Cy- may be independently selected from:

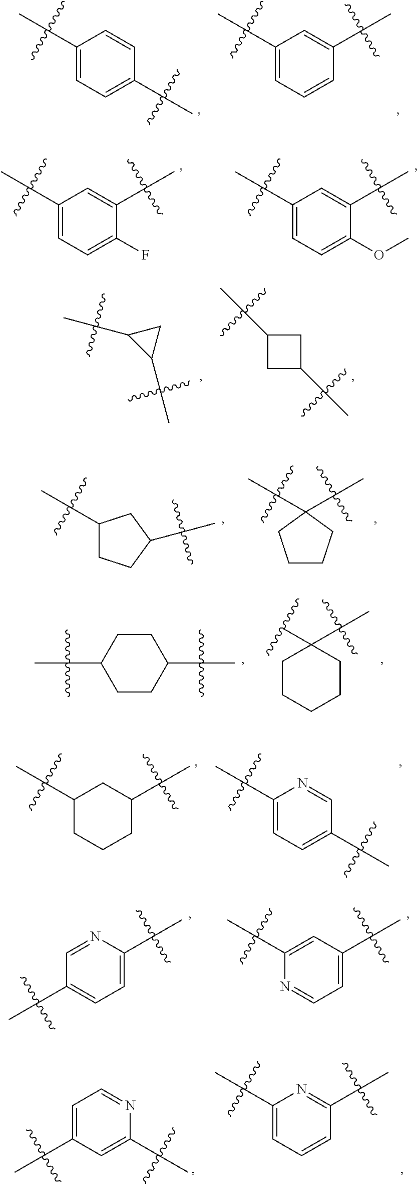

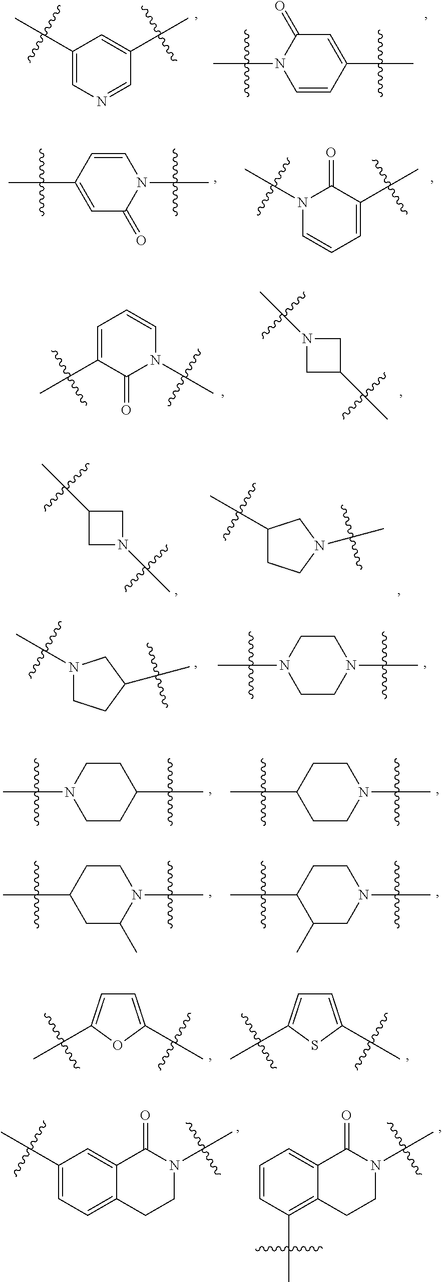

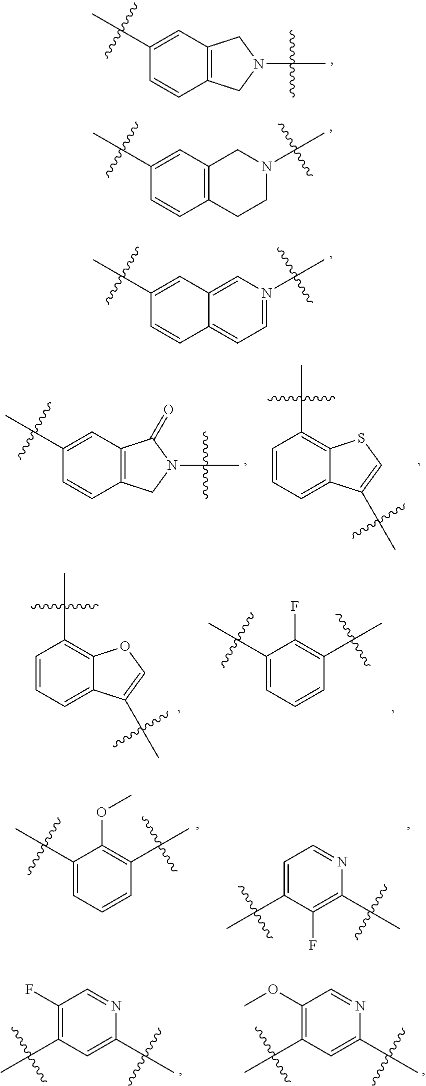

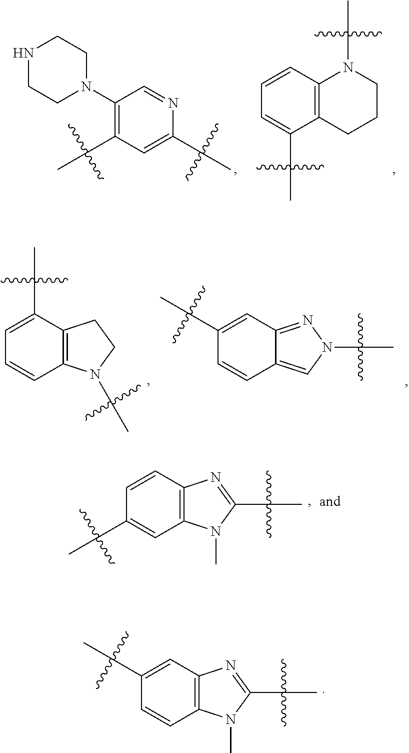 [SMILES: *c1cc(*)c(N2CCNCC2)cn1 ; *c1cccc2c1CCCN2* ; *c1cccc2c1CCN2* ; *c1ccc2cn(*)nc2c1 ; *c1ccc2nc(*)n(C)c2c1 ; *c1ccc2c(c1)nc(*)n2C]

Specifically, R^{L1} may be selected from: H. C₁₋₆ alkyl, wherein one or more H in the alkyl may be substituted with a substituent selected from: halogen (particularly F), —OH, —NH₂, C₁₋₆ alkoxy (e.g., methoxy, or ethoxy), and C₁₋₆ alkylamino

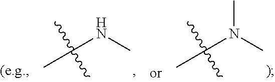

more specifically, R^{L1} may be selected from: H, C₁₋₃ alkyl,

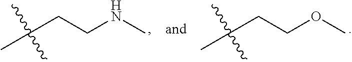

Specifically, R^{L1′} and R^{L″} may be independently selected from: halogen, and C₁₋₆ alkyl, wherein one or more H in the alkyl may be substituted with a substituent selected from: halogen (particularly F), —OH, —NH₂, C₁₋₆ alkoxy (e.g., methoxy, or ethoxy), and C₁₋₆ alkylamino

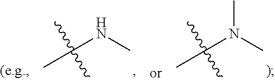

more specifically, R^{L1′} and R^{L″} may be independently selected from: H, C₁₋₃ alkyl,

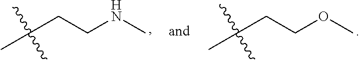

Specifically, R^{L1′} and R^{L1″}, together with carbon atoms linked thereto, may form C₃₋₆ cycloalkyl, e.g.,

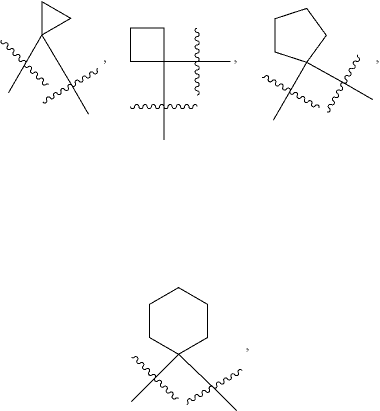 [SMILES: *C1(*)CC1 ; *C1(*)CCC1 ; *C1(*)CCCC1 ; *C1(*)CCCCC1]

or 4- to 6-membered heterocyclyl, e.g.,

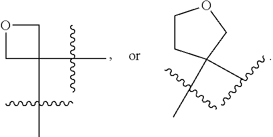 [SMILES: *C1(*)COC1 ; *C1(*)CCOC1]

In some embodiments of the present invention, L₁ is —NR^{L1}—(C₀₋₆ alkylene)-, —O—(C₀₋₆ alkylene)-, —S—(C₀₋₆ alkylene)-, —(C₀₋₆ alkylene)-,

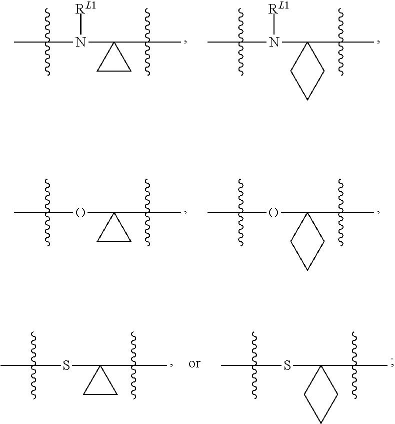

for example, L₁ may be —NH—, —NH—CH₂—, —NH—CH₂CH₂—, —NH—CH₂CH₂CH₂—,

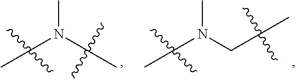

—O—, —O—CH₂—, —O—CH₂CH₂—, —O—CH₂CH₂CH₂—, —S—, —S—CH₂—, —S—CH₂CH₂—, —S—CH₂CH₂CH₂—, single bond, —CH₂—, —CH₂CH₂—, —CH₂CH₂CH₂—, or

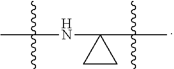 [SMILES: *NC1(*)CC1]

Specifically, R^{L3} may be selected from: H, C₁₋₆ alkyl, wherein one or more H in the alkyl may be substituted with a substituent selected from: halogen (particularly F), —OH, —NH₂, C₁₋₆ alkoxy (e.g., methoxy, or ethoxy), and C₁₋₆ alkylamino

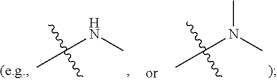

more specifically, R^{L3} may be selected from: H, C₁₋₃ alkyl,

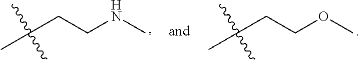

Specifically, R^{L3′} and R^{L3″} may be independently selected from: halogen, and C₁₋₆ alkyl, wherein one or more H in the alkyl may be substituted with a substituent selected from: halogen (particularly F), —OH, —NH₂, C₁₋₆ alkoxy (e.g., methoxy, or ethoxy), and C₁₋₆ alkylamino

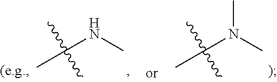

more specifically, R^{L3′} and R^{L3″} may be independently selected from: H, C₁₋₃ alkyl,

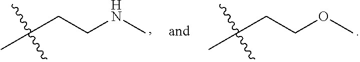

Specifically, R^{L3′} and R^{L3″}, together with carbon atoms linked thereto, may form C₃₋₆ cycloalkyl, e.g.,

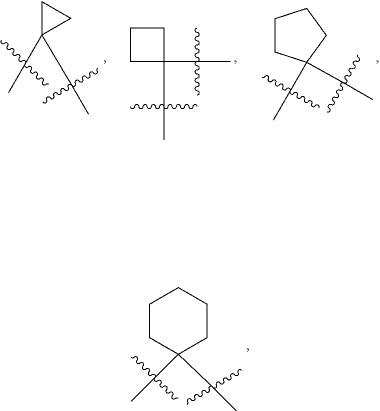 [SMILES: *C1(*)CC1 ; *C1(*)CCC1 ; *C1(*)CCCC1 ; *C1(*)CCCCC1]

or 4- to 6-membered heterocyclyl, e.g.,

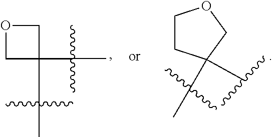 [SMILES: *C1(*)COC1 ; *C1(*)CCOC1]

In some embodiments of the present invention, L₃ is —(C₀₋₆ alkylene)-NR^{L3}CO—, —(C₀₋₆ alkylene)-, CONR^{L3}—, —(C₀₋₆ alkylene)-CO—, —(C₀₋₆ alkylene)-NH—, —(C₀₋₆ alkylene)-,

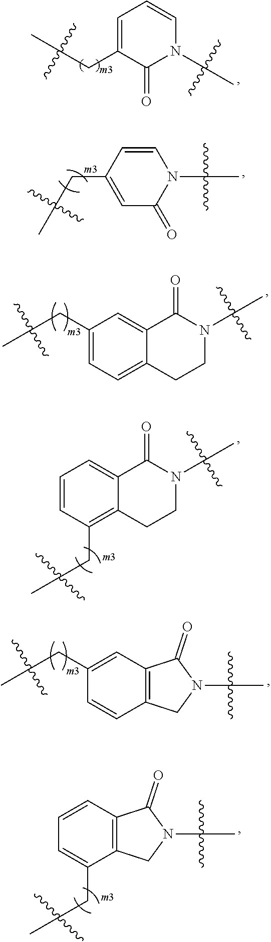

—NR^{L3}—(C₀₋₆ alkylene)-, —O—(C₀₋₆ alkylene)-, —S—(C₀₋₆ alkylene)-, —NR^{L3}CO—(C₀₋₆ alkylene)-, —CONR^{L3}—(C₀₋₆ alkylene)-, or —CO—(C₀₋₆ alkylene)-; for example, L₃ may be —CONH—, —CH₂—CONH—, —CH₂CH₂—CONH—, —CH₂CH₂CH₂

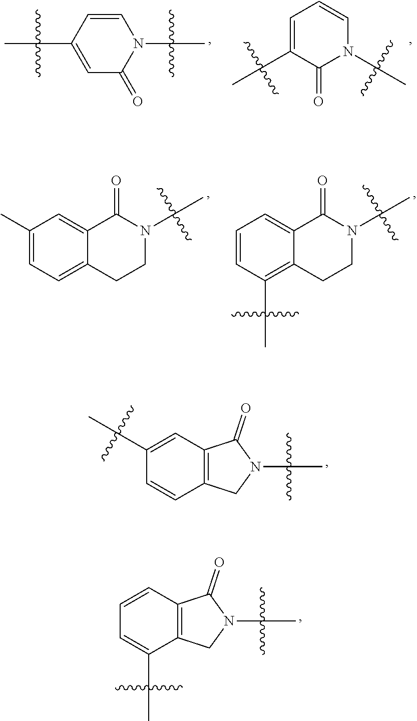 [SMILES: *c1ccn(*)c(=O)c1 ; *c1cccn(*)c1=O ; *N1CCc2ccc(C)cc2C1=O ; *c1cccc2c1CCN(*)C2=O ; *c1ccc2c(c1)C(=O)N(*)C2 ; *c1cccc2c1CN(*)C2=O]

—NH—, —CH₂—NH—, —CH₂CH₂—NH—, —CH₂CH₂CH₂—NH—, single bond,

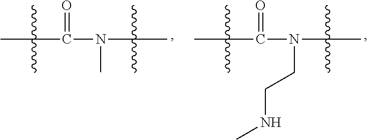

### —NHCO—,

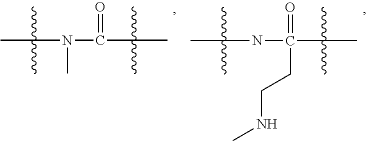

—NHCO—CH₂—, —CO—, —CO—CH₂—, —NH—CH₂—,

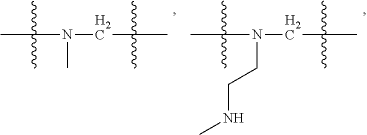

—O—CH₂—, or —S—CH₂—.

Specifically, L₂ may be selected from: C₁-C₂₀ linear alkylene (e.g., C₁, C₂, C₃, C₄, C₅, C₆, C₇, C₈, C₉, C₁₀, C₁₁, C₁₂, C₁₃, C₁₄, C₁₅, C₁₆, C₁₈, or C₂₀ linear alkylene), —(CH₂CH₂O)ₘ₂—, and -Cy-, wherein, m2 is an integer selected from 0-5 (e.g., 0, 1, 2, 3, 4, or 5).

In some embodiments of the present invention, L is selected from:

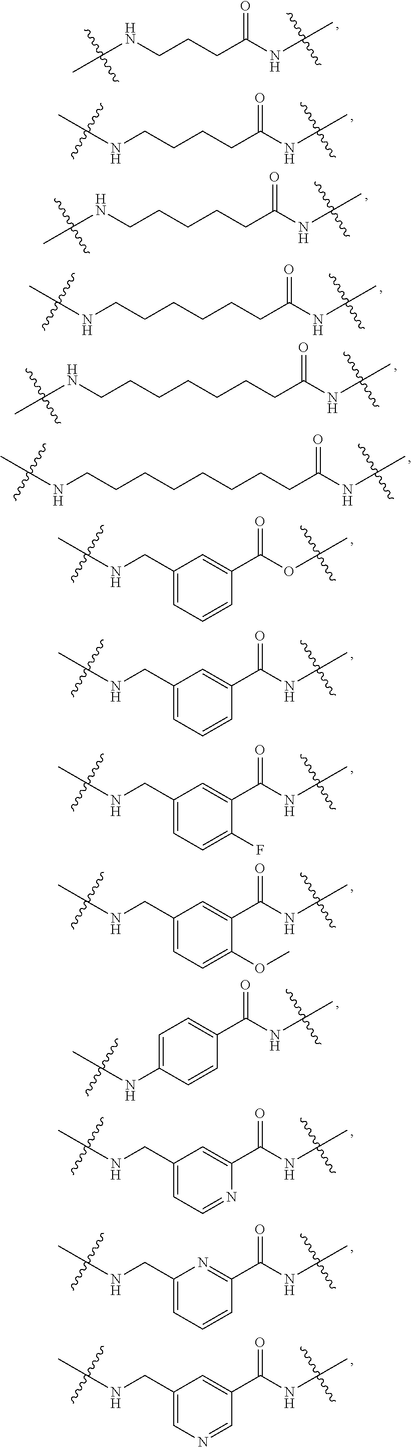

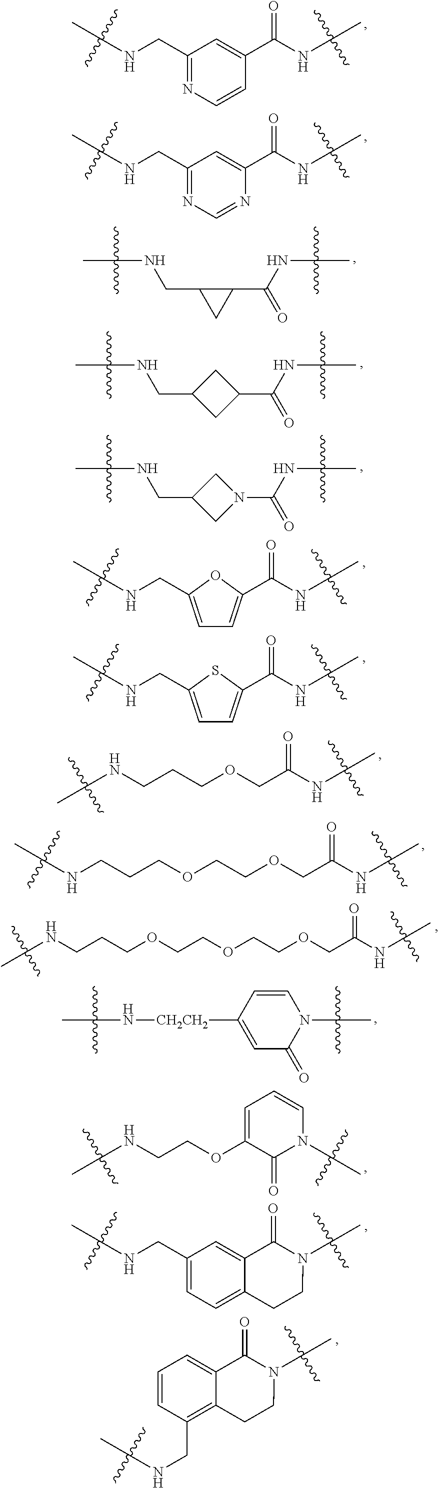

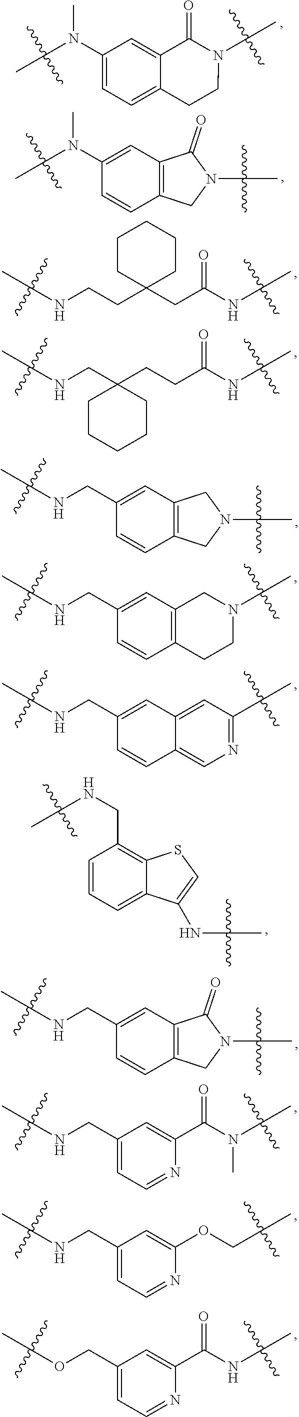

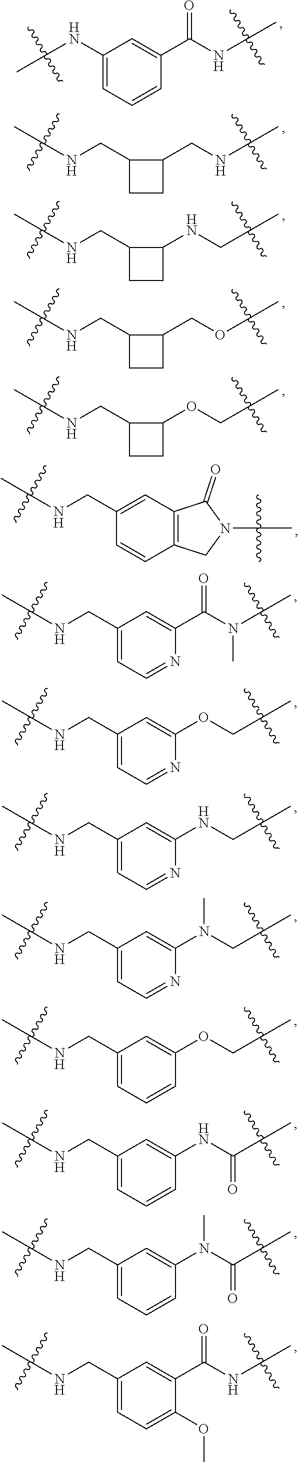

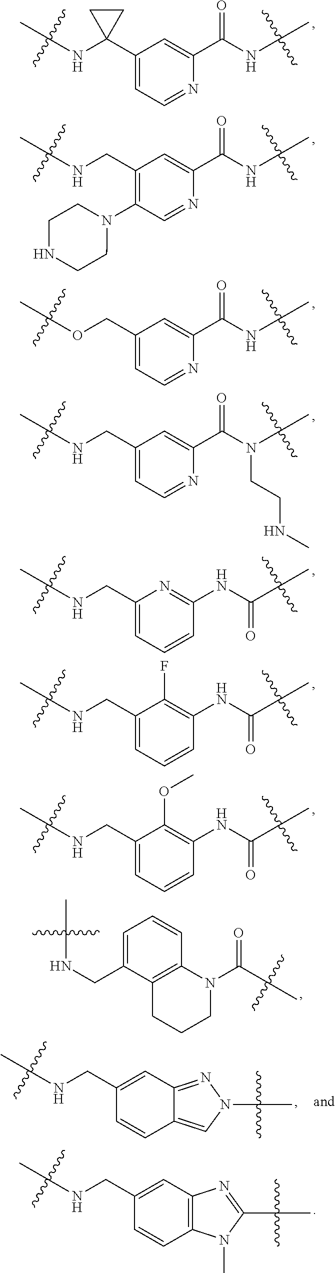

Specifically, ring A may be an aliphatic ring, an aromatic ring or a heterocyclic ring, e.g., a monocyclic saturated aliphatic ring, a bicyclic saturated aliphatic ring, a monocyclic aromatic ring, a bicyclic aromatic ring, a monocyclic heterocyclic ring, or a bicyclic heterocyclic ring (e.g., a benzene ring, an 8- to 10-membered bicyclic aromatic ring, a 3- to 7-membered monocyclic saturated aliphatic ring, a 4- to 7-membered saturated spiro aliphatic ring, an 8- to 10-membered saturated fused aliphatic ring, an 8- to 10-membered saturated bridged aliphatic ring, a 4- to 7-membered monocyclic saturated heterocyclic ring, a 4- to 7-membered saturated spiro heterocyclic ring, an 8- to 10-membered saturated fused heterocyclic ring, or an 8- to 10-membered saturated bridged heterocyclic ring), particularly a saturated carbocyclic or heterocyclic ring (e.g., a 3- to 7-membered monocyclic saturated carbocyclic ring, a 4- to 7-membered saturated spiro carbocyclic ring, an 8- to 10-membered fused carbocyclic ring, an 8- to 10-membered saturated bridged carbocyclic ring, a 4- to 7-membered monocyclic saturated heterocyclic ring, a 4- to 7-membered saturated spiro heterocyclic ring, an 8- to 10-membered saturated fused heterocyclic ring, or an 8- to 10-membered saturated bridged heterocyclic ring); in some embodiments of the present invention, moiety

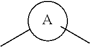

is a 3- to 7-membered monocyclic saturated carbocyclylene, a 4- to 7-membered saturated spiro carbocyclylene, an 8- to 10-membered saturated fused carbocyclylene, a 3- to 7-membered monocyclic saturated heterocyclylene, a 4- to 7-membered saturated spiro heterocyclylene, or an 8- to 10-membered saturated fused heterocyclylene.

More specifically, moiety

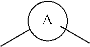

may be selected from:

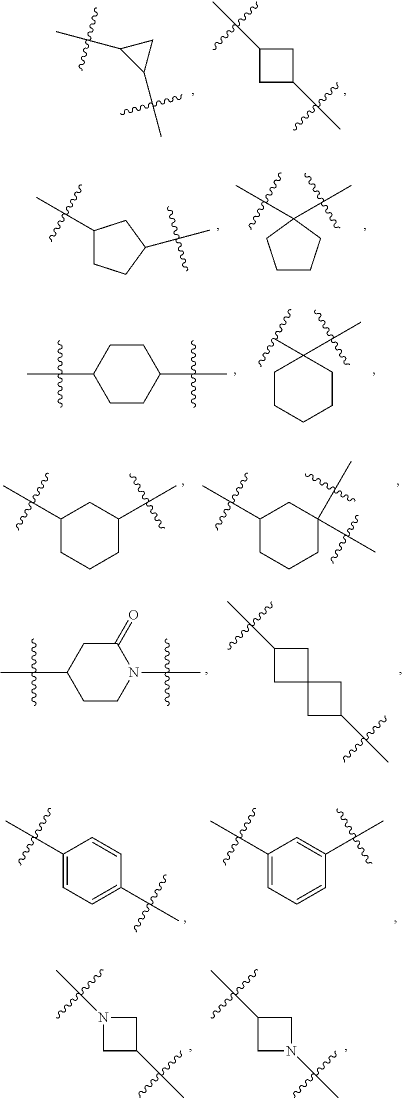

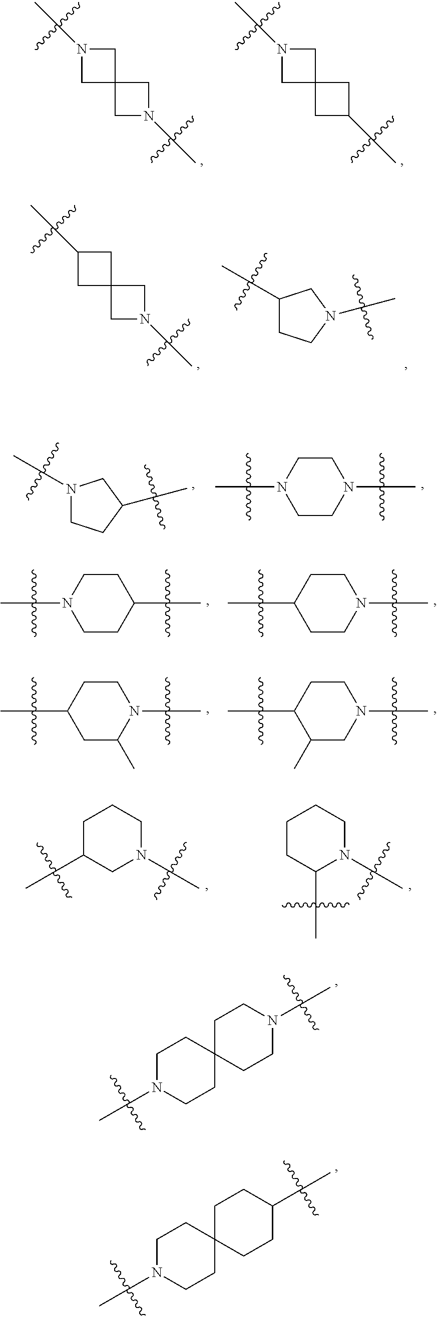

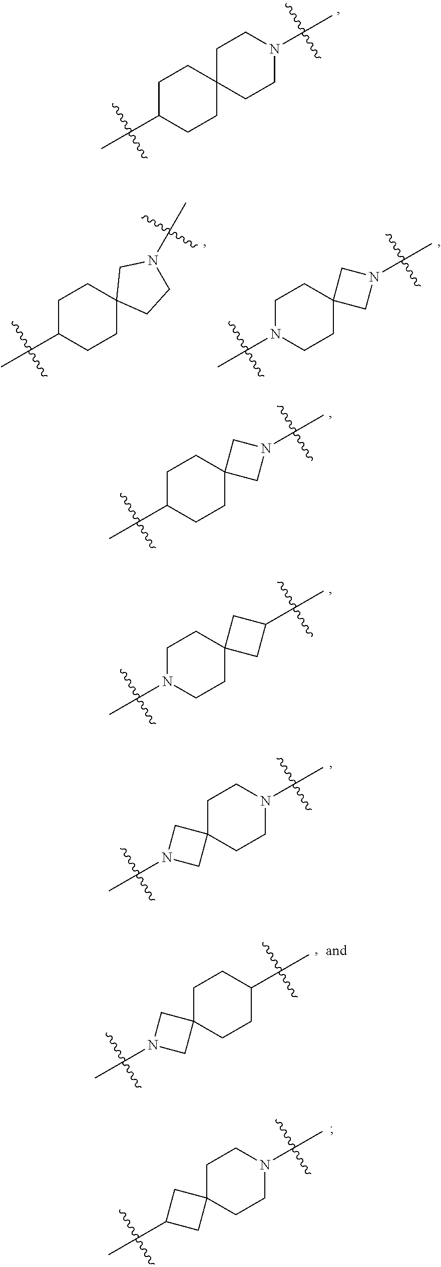

in some embodiments of the present invention, moiety

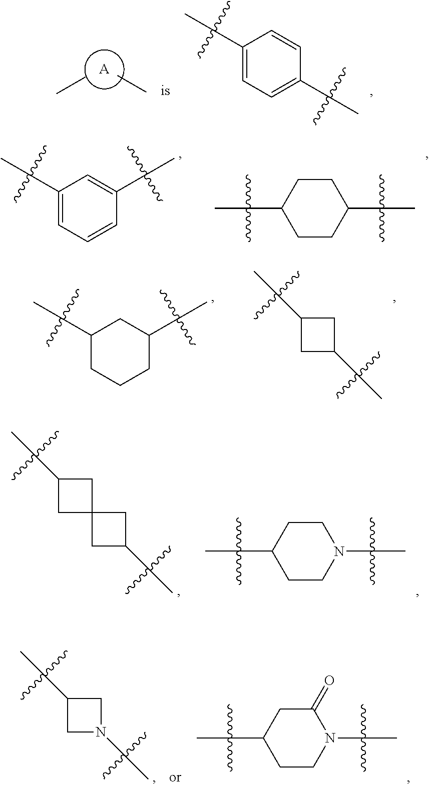

particularly

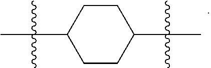 [SMILES: *C1CCC(*)CC1]

In one embodiment of the present invention, in the compound described above, R₁ is one or more independent substituents of ring A and is selected from: H, halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), and —CO(C₀₋₁₀ alkyl), and L₂ is -Cy-; more specifically, moiety

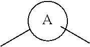

is 3- to 7-membered monocyclic saturated carbocyclylene, 4- to 7-membered saturated spiro carbocyclylene, 8- to 10-membered saturated fused carbocyclylene, 3- to 7-membered monocyclic saturated heterocyclylene, 4- to 7-membered saturated spiro heterocyclylene, or 8- to 10-membered saturated fused heterocyclylene, e.g.,

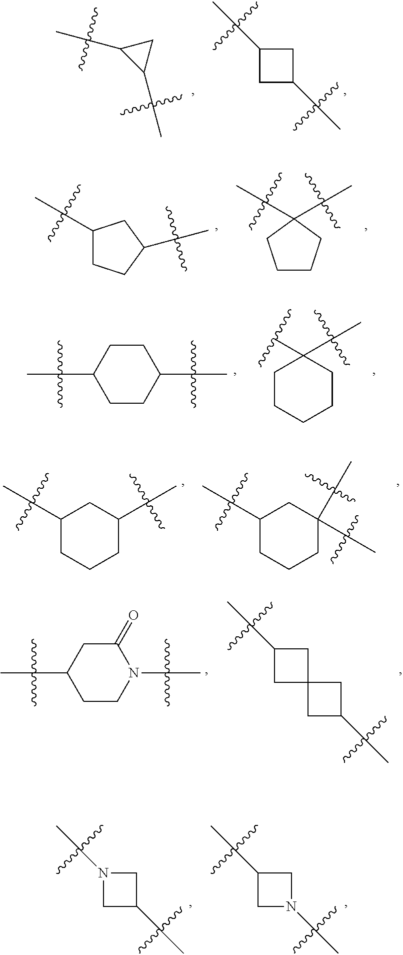

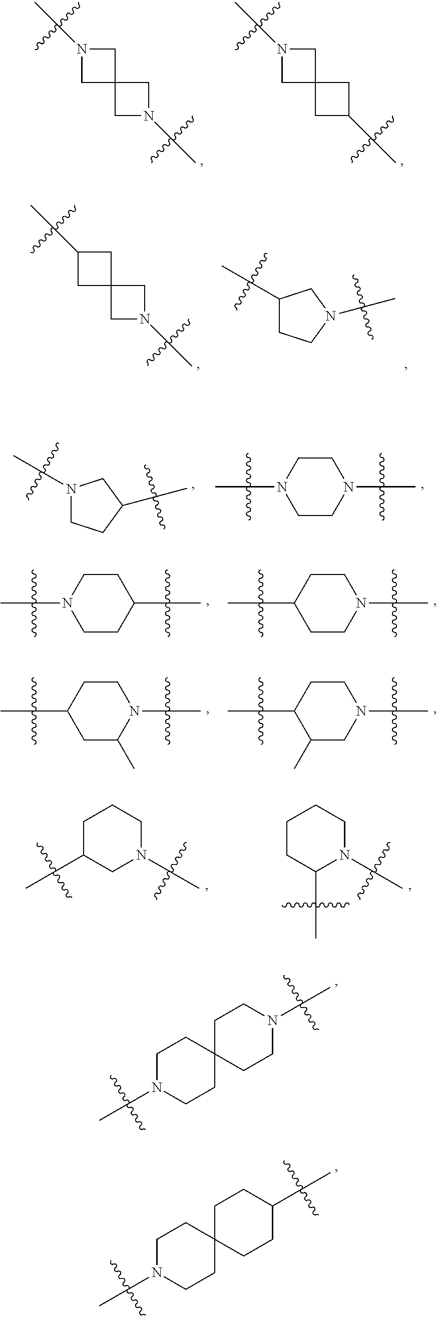

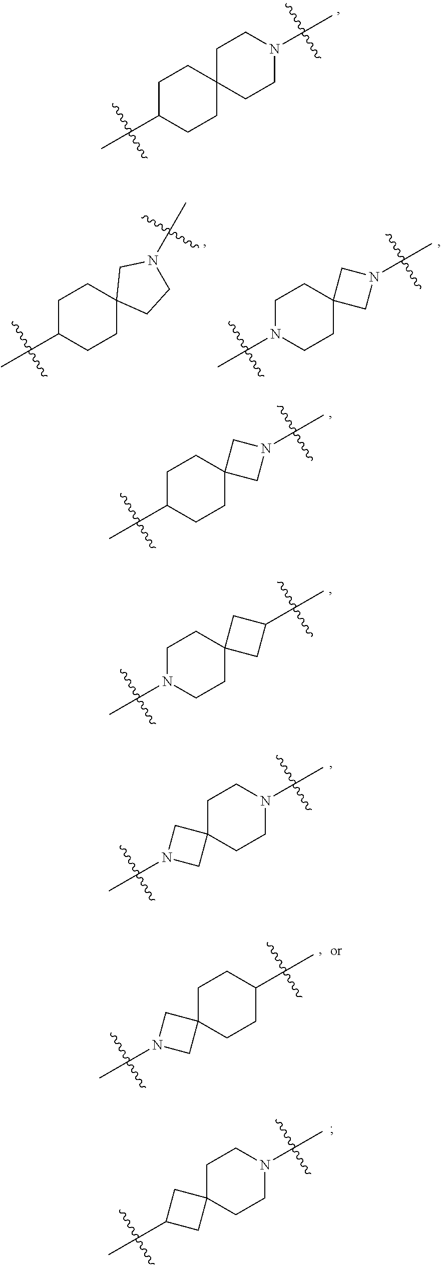

-Cy- may be substituted or unsubstituted arylene or heteroarylene, e.g., substituted or unsubstituted phenylene, e.g.,

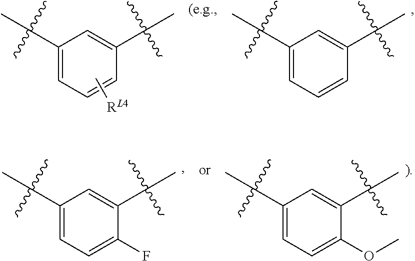

In some embodiments of the present invention, moiety

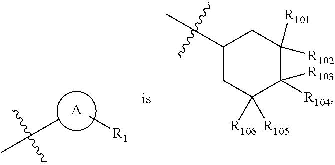

wherein R₁₀₁, R₁₀₂, R₁₀₃, R₁₀₄, R₁₀₅ and R₁₀₆ each have the definition for R₁; specifically, R₁₀₃, R₁₀₄, R₁₀₅ and R₁₀₆ may all be H

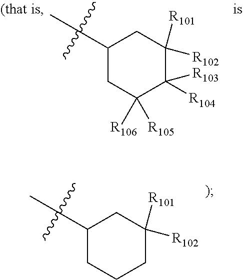

specifically, R₁₀₁ and R₁₀₂ are independently selected from: —H, halogen (e.g., F), —OH, C₁₋₆ alkyl (e.g., —CH₃, or —CH₂CH₃), and —O(C₁₋₆ alkyl)(e.g., methoxy, or ethoxy); more specifically,

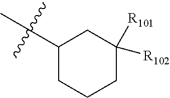

may be, for example,

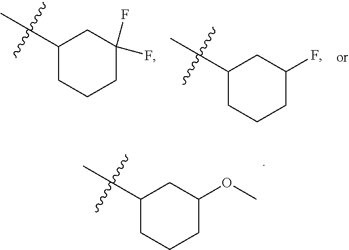

In another embodiment of the present invention, R₁ is

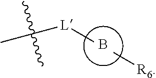

In some embodiments of the present invention, the compound described above may have the following structure:

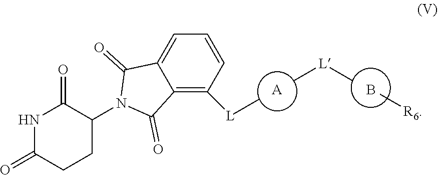

In one embodiment of the present invention, L′ is a single bond; that is, R₆ is

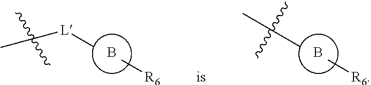

Specifically, ring B may be an aromatic ring or a heteroaromatic ring, e.g., a benzene ring, a bicyclic aromatic ring, a tricyclic aromatic ring, a monocyclic heteroaromatic ring, a bicyclic heteroaromatic ring, or a tricyclic heteroaromatic ring.

In one embodiment of the present invention, ring B is a benzene ring, and moiety

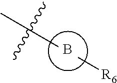

may be

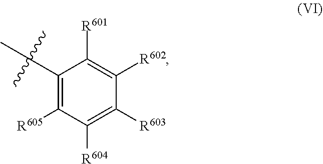

wherein R⁶⁰¹, R⁶⁰², R⁶⁰³, R⁶⁰⁴ and R⁶⁰⁵ each have the definition for R₆ and are independently selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), cycloalkyl, heterocycloalkyl, and heteroaryl, wherein, H at the carbon atoms may be substituted with one or more groups selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl.

Specifically, in formula VI, R⁶⁰¹, R⁶⁰², R⁶⁰³, R⁶⁰⁴ and R⁶⁰⁵ are independently selected from: —H, halogen, —OH, C₁₋₆ alkyl (e.g., —CH₃, —CH₂CH₃), and —O(C₁₋₆ alkyl)(e.g., methoxy, or ethoxy); in some embodiments of the present invention, R⁶⁰¹, R⁶⁰², R⁶⁰³, R⁶⁰⁴ and R⁶⁰⁵ are all —H.

In another embodiment of the present invention, ring B is a monocyclic heteroaromatic ring; for example, moiety

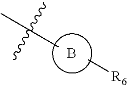

may be

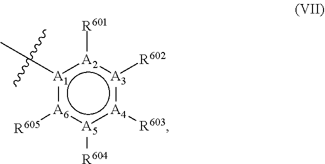

wherein A₁, A₂, A₃, A₄, A₅ and A₆ are independently selected from C and N, and at least one of A₁, A₂, A₃, A₄, A₅ and A₆ is N; when any one of A₂, A₃, A₄, A₅ and A₆ is N, R⁶⁰¹, R⁶⁰², R⁶⁰³, R⁶⁰⁴ and R⁶⁰⁵ corresponding thereto is absent; R⁶⁰¹, R⁶⁰², R⁶⁰³, R⁶⁰⁴ and R⁶⁰⁵ each have the definition as described above for R₆ and are independently selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl, wherein H at the carbon atoms may be substituted with one or more groups selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl.

Specifically, in the formula VII, one of A₁, A₂, A₃, A₄, A₅ and A₆ is N, and the remainder are C; or two of A₁, A₂, A₃, A₄, A₅, A₆ are N, and the remainder are C; three of A₁, A₂, A₃, A₄, A₅ and A₆ are N, and the remainder are C.

Further specifically, moiety

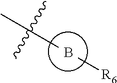

may have the following structures:

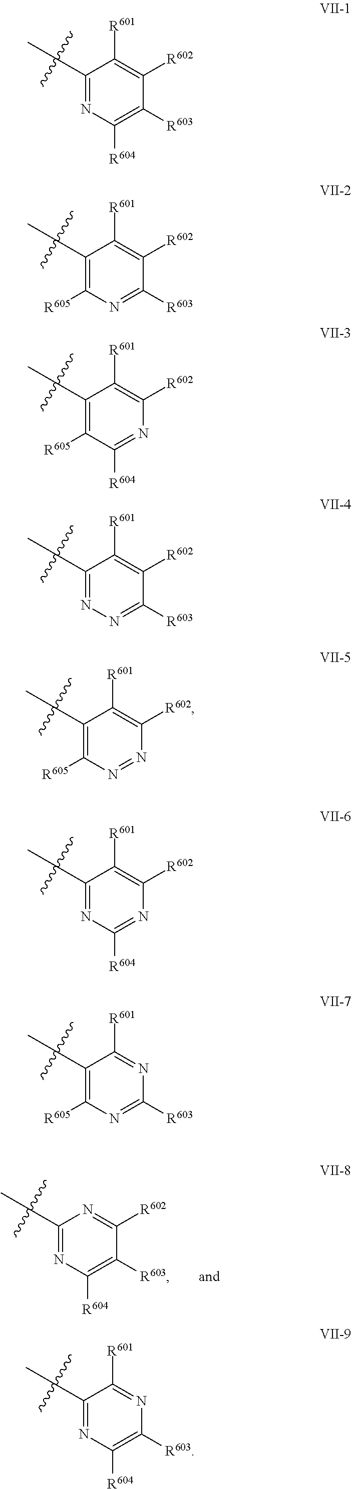

More specifically, in the formula VII, R⁶⁰¹, R⁶⁰², R⁶⁰³ and R⁶⁰⁴ are independently selected from: —H, -D, —F, —Cl, —Br, —I, —CF₃, —CHF₂, —CH₂F, —CH₂CF₃, —CN, —NO₂, —CH₃,

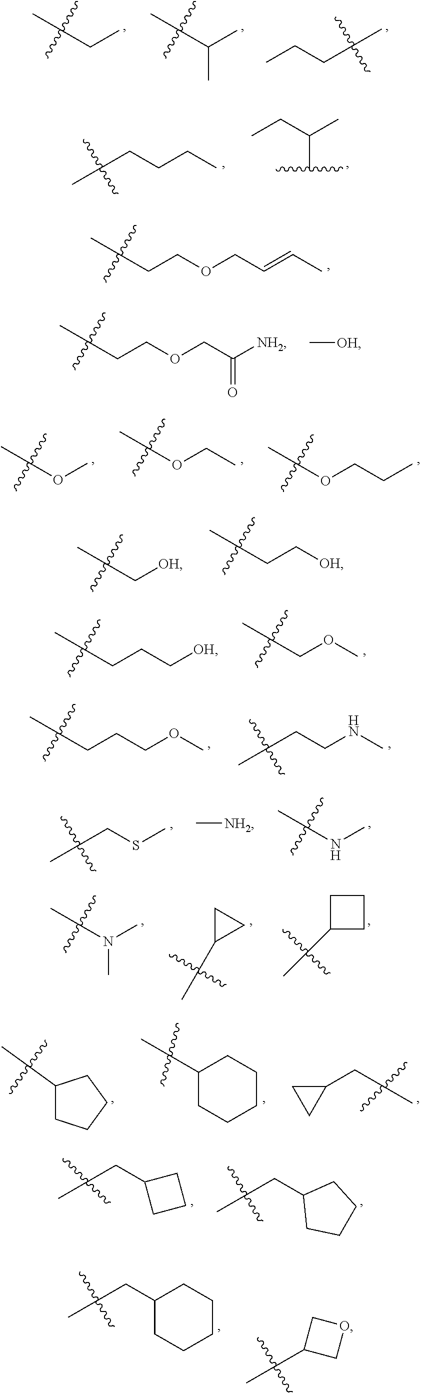

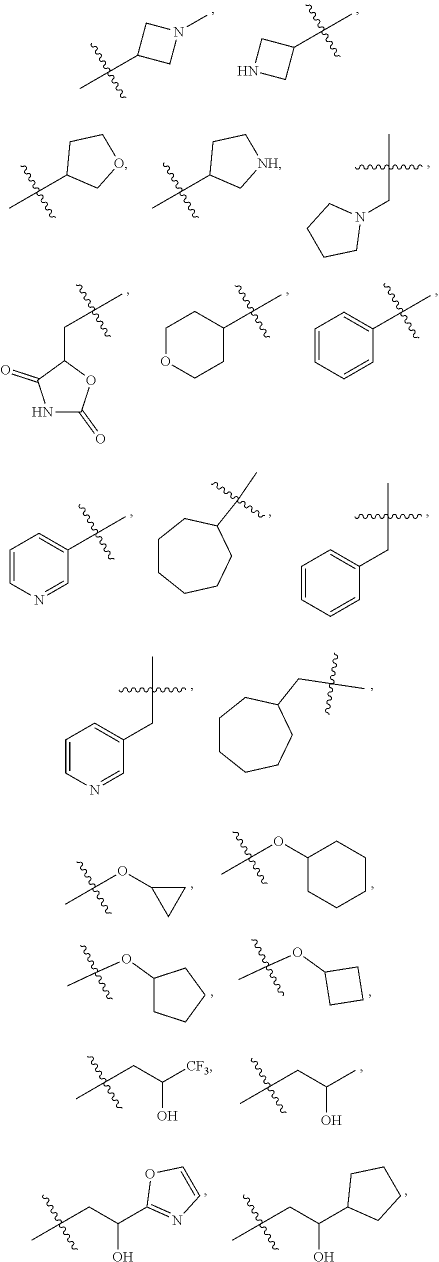

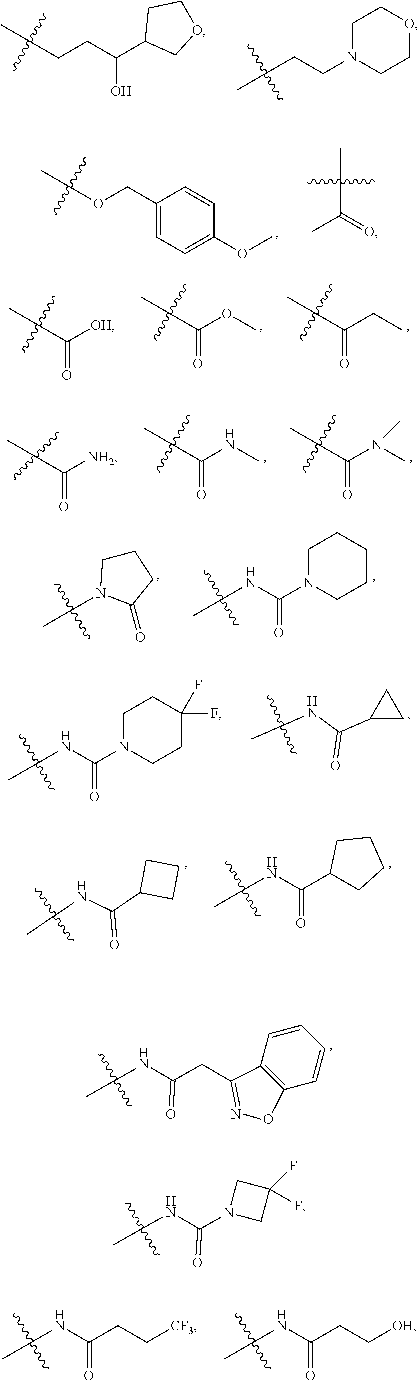

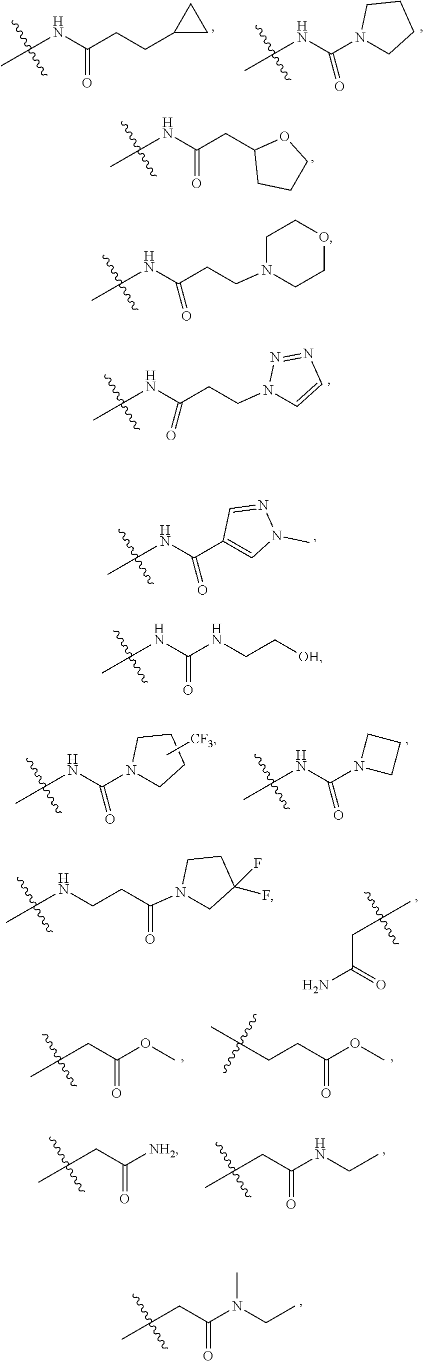

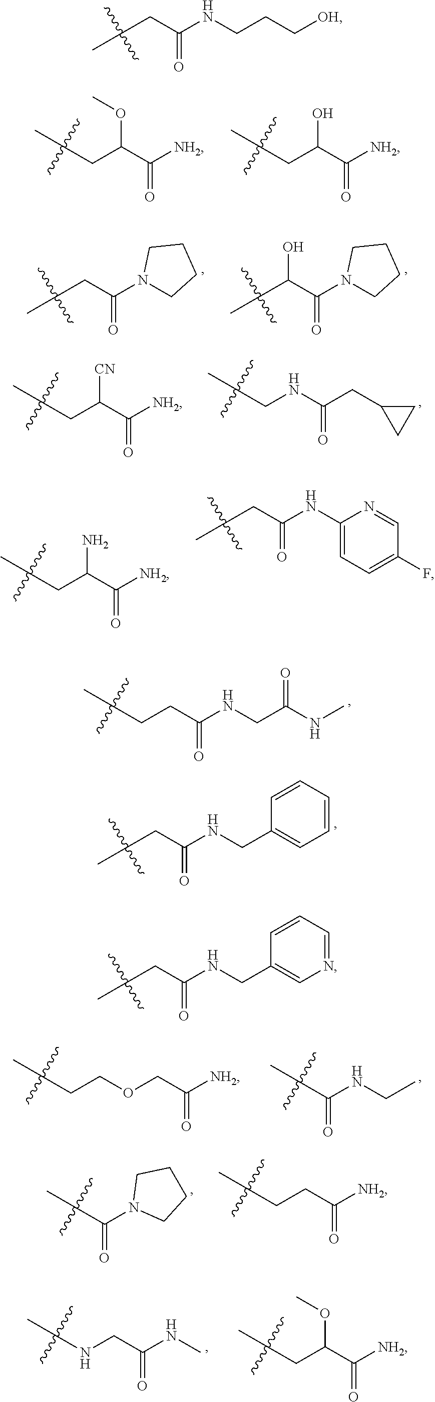

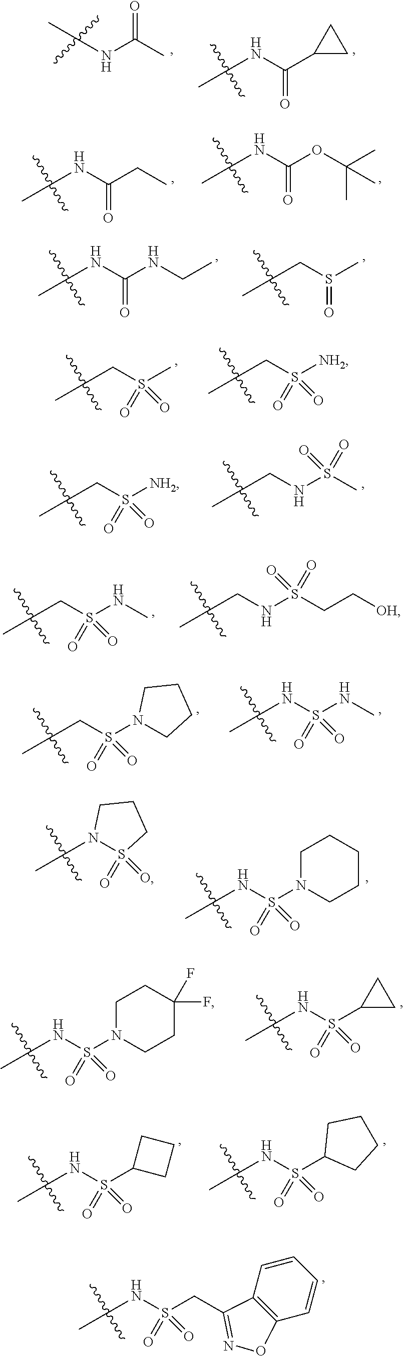

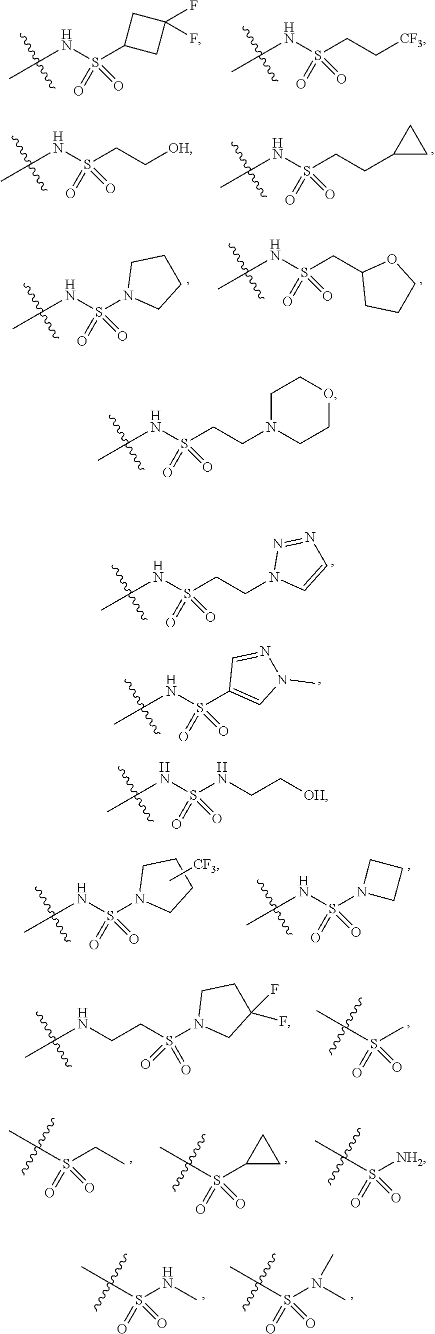

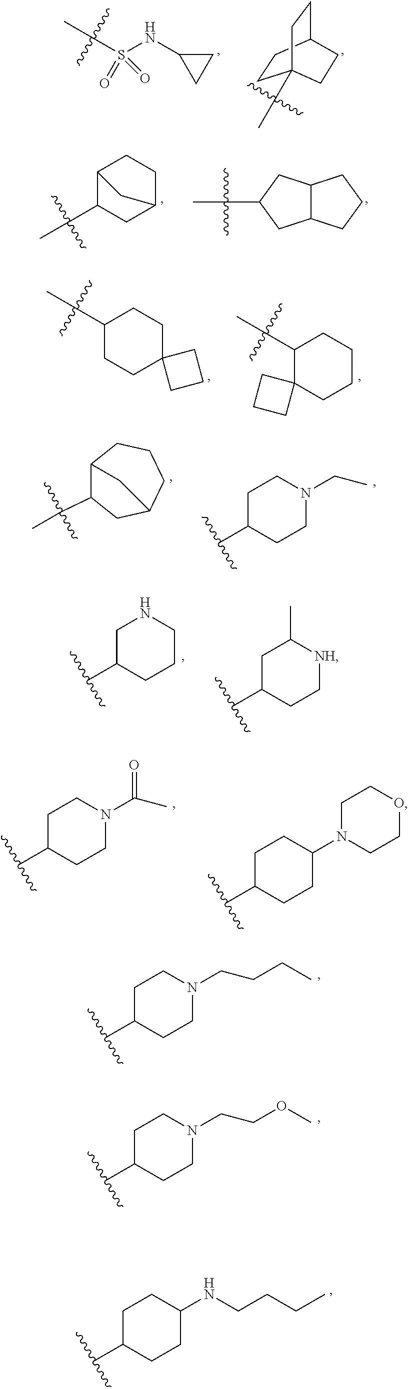

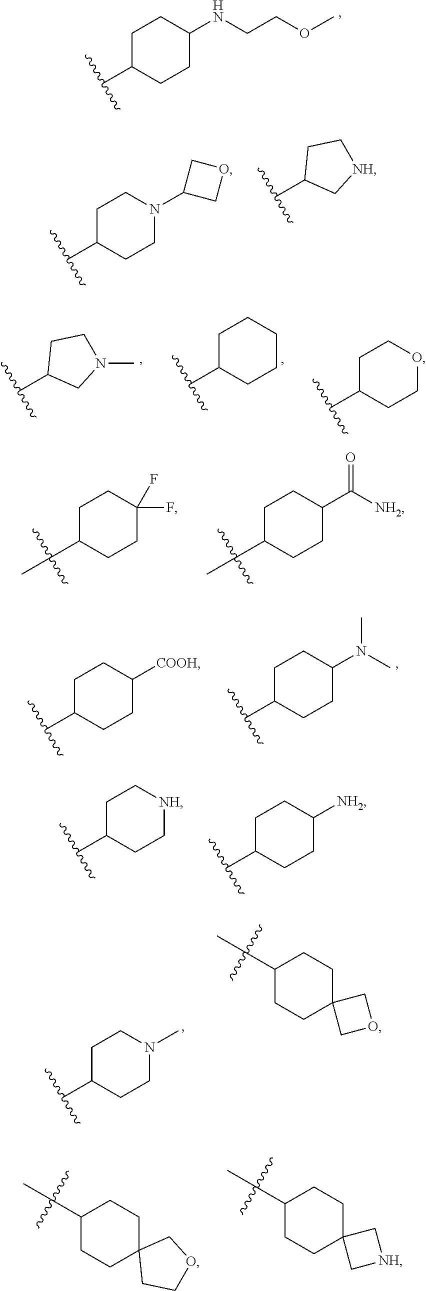

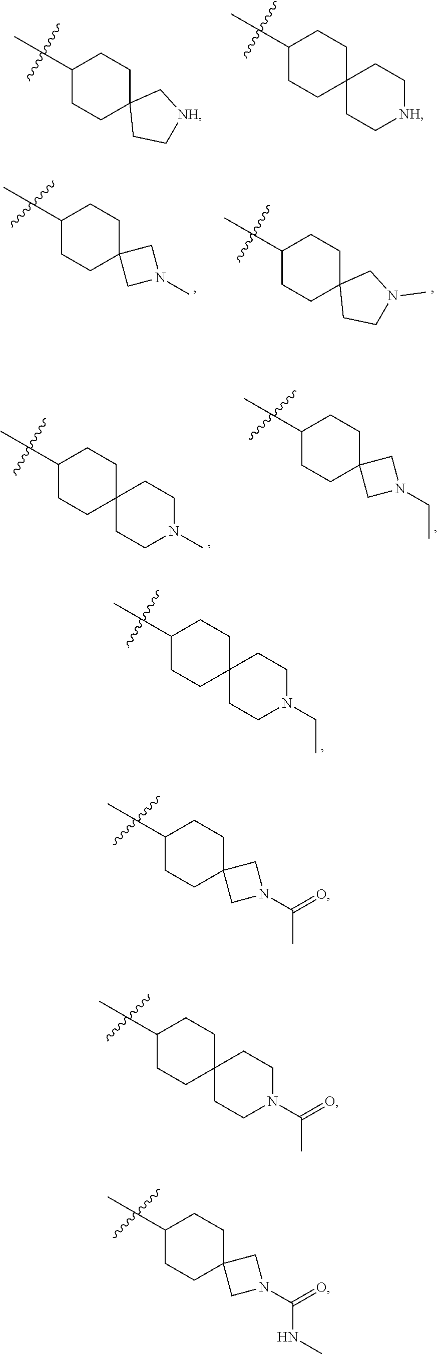

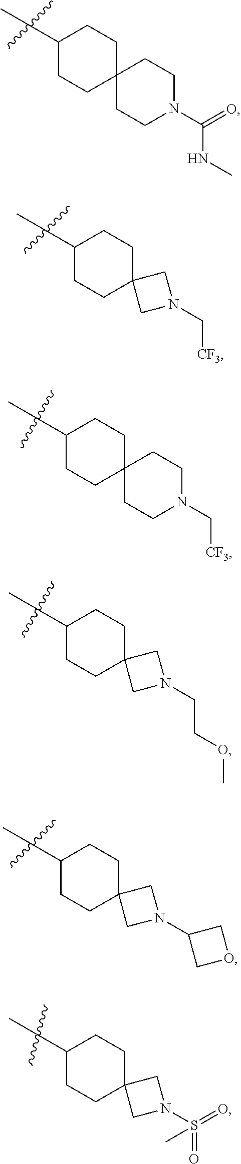 [SMILES: *C1CCC2(CC1)CCN(C(=O)NC)CC2 ; *C1CCC2(CC1)CN(CC(F)(F)F)C2 ; *C1CCC2(CC1)CCN(CC(F)(F)F)CC2 ; *C1CCC2(CC1)CN(CCOC)C2 ; *C1CCC2(CC1)CN(C1COC1)C2 ; *C1CCC2(CC1)CN(S(C)(=O)=O)C2]

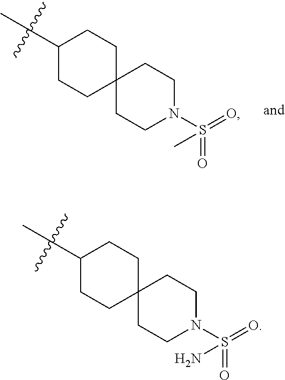 [SMILES: *C1CCC2(CC1)CCN(S(C)(=O)=O)CC2 ; *C1CCC2(CC1)CCN(S(N)(=O)=O)CC2]

In another embodiment of the present invention, ring B is a bicyclic heteroaromatic ring.

Specifically, moiety

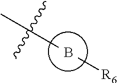

may have the following structure:

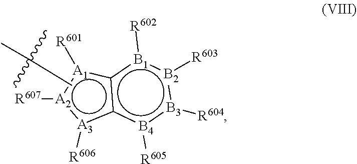

wherein A₁, A₂ and A₃ are independently selected from C, N, O and S; B₁, B₂ and B₃ are independently selected from C and N; B₄ is C, N or absent, and satisfies valence-bond saturation; R⁶⁰¹, R⁶⁰², R⁶⁰³, R⁶⁰⁴, R⁶⁰⁵, R⁶⁰⁶ and R⁶⁰⁷ may be appropriately absent; R⁶⁰¹, R⁶⁰², R⁶⁰³, R⁶⁰⁴, R⁶⁰⁵, R⁶⁰⁶ and R⁶⁰⁷ are independently selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl, wherein H at the carbon atoms may be substituted with one or more groups selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl.

In some embodiments of the present invention, B₁, B₂, B₃ and B₄ are all C; or B₁ is N, and B₂, B₃ and B₄ are all C; or B₂ is N, and B₁, B₃ and B₄ are all C; or B₃ is N, and B₁, B₂ and B₄ are all C; or B₄ is N, and B₂, B₃ and B₁ are all C.

More specifically, moiety

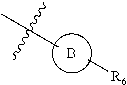

may have the following structure:

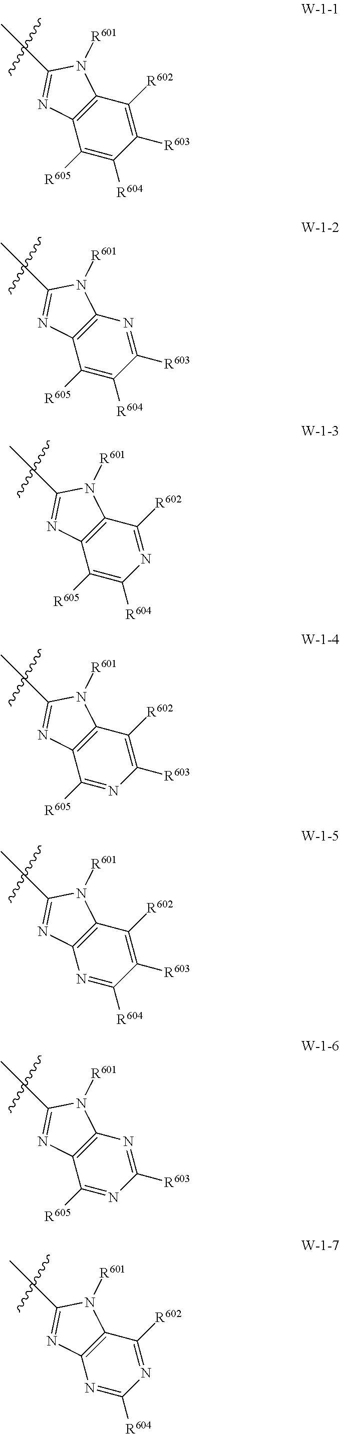

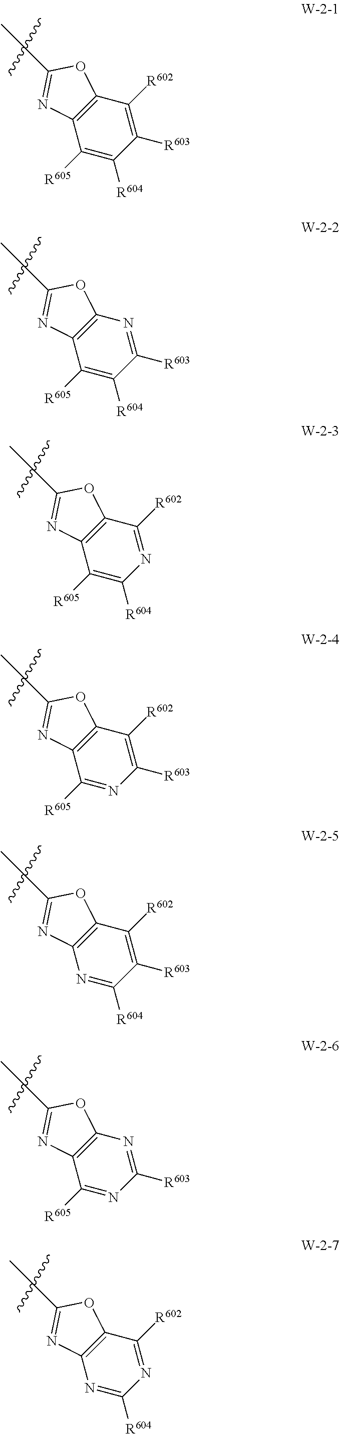

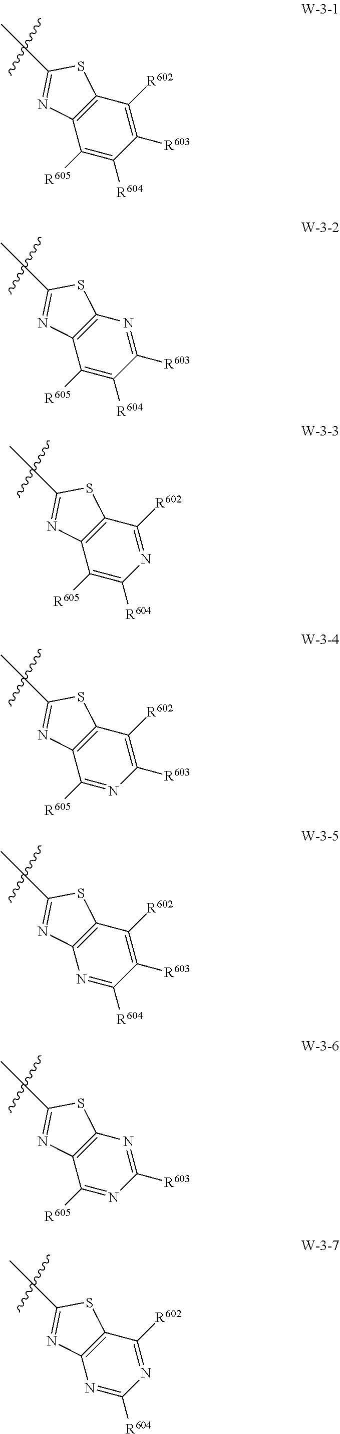

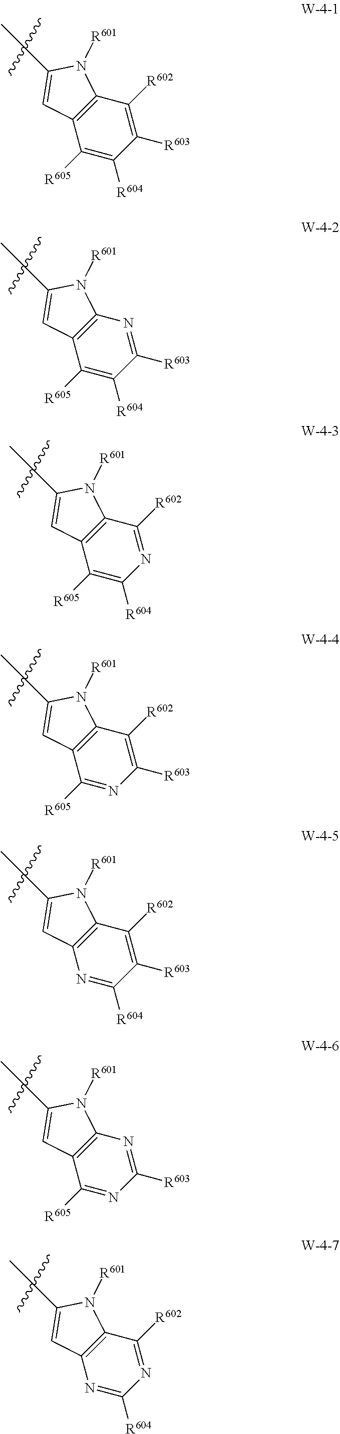

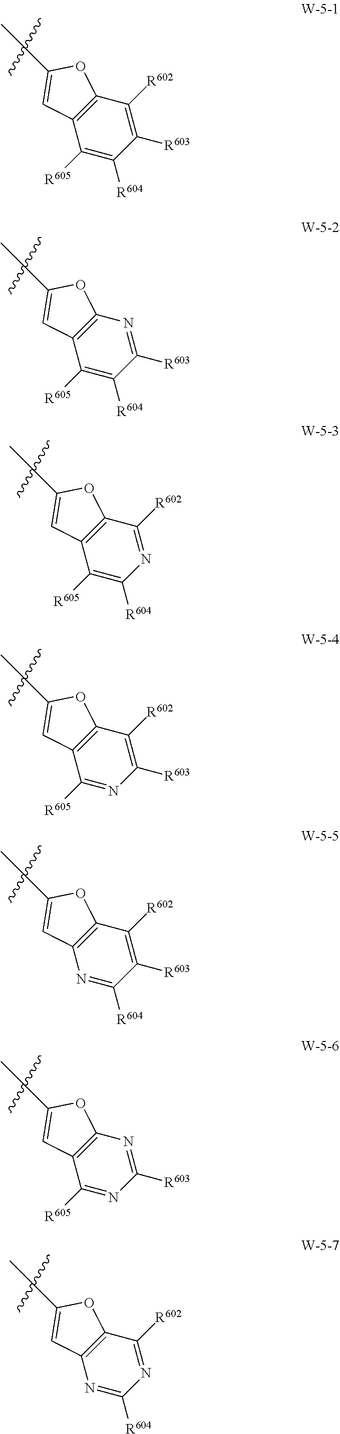

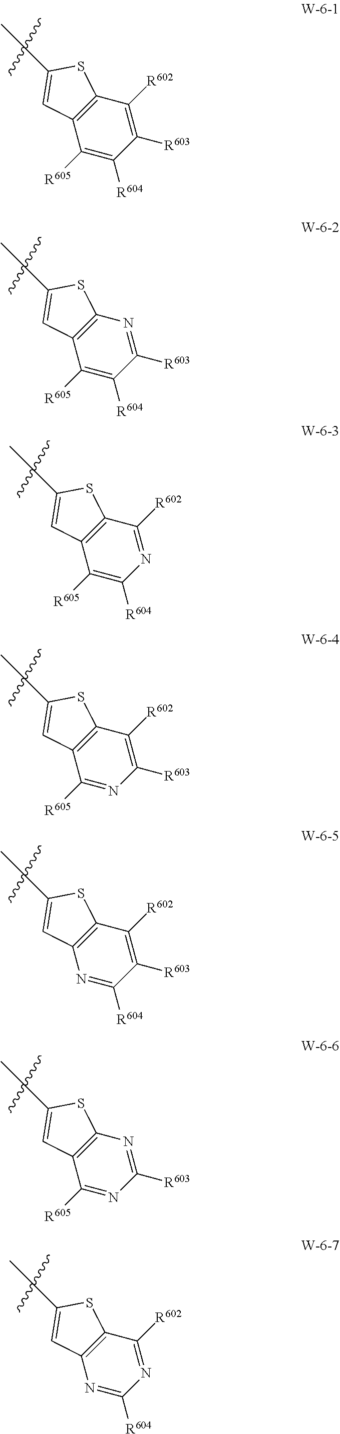

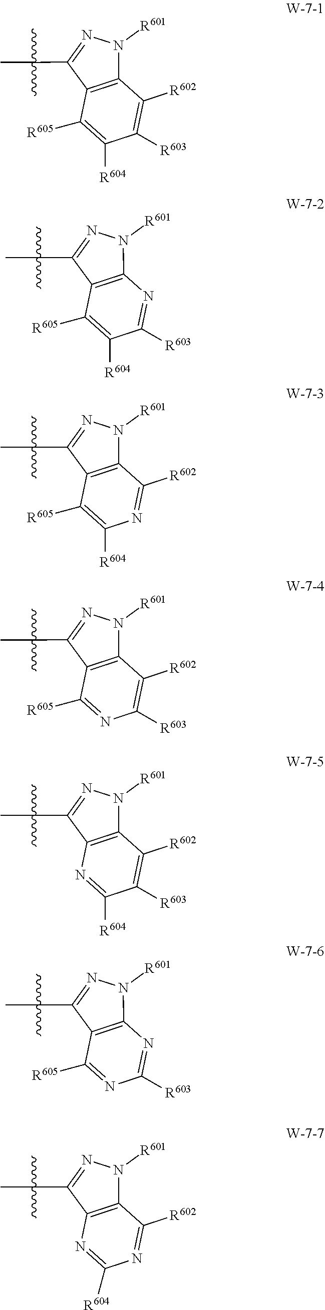

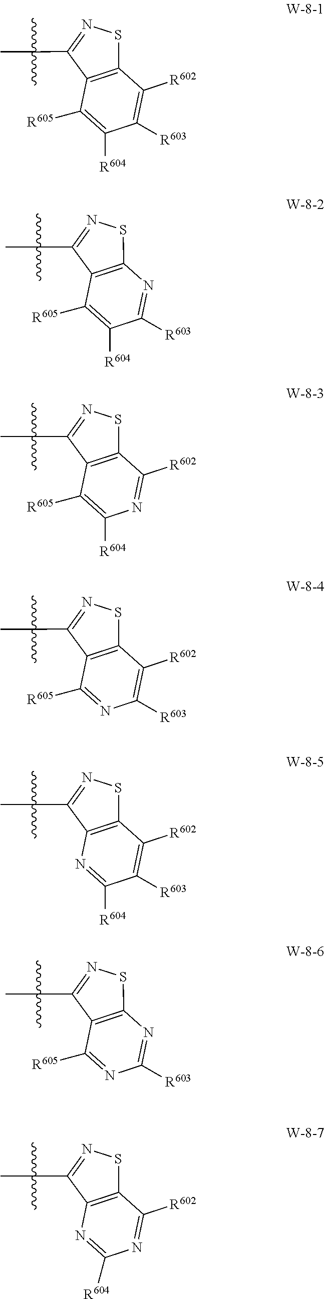

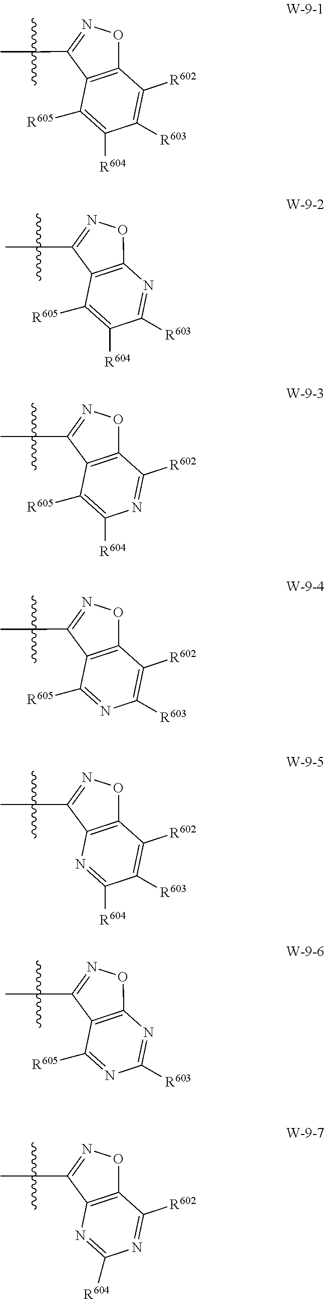

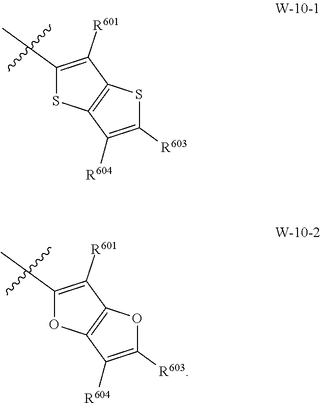

More specifically, in formula VIII, R⁶⁰¹, R⁶⁰², R⁶⁰³, R⁶⁰⁴, R⁶⁰⁵, R⁶⁰⁶ and R⁶⁰⁷ are independently selected from: —H, -D, —F, —Cl, —Br, —I, —CF₃, —CHF₂, —CH₂F, —CH₂CF₃, —CN, —NO₂, —CH₃,

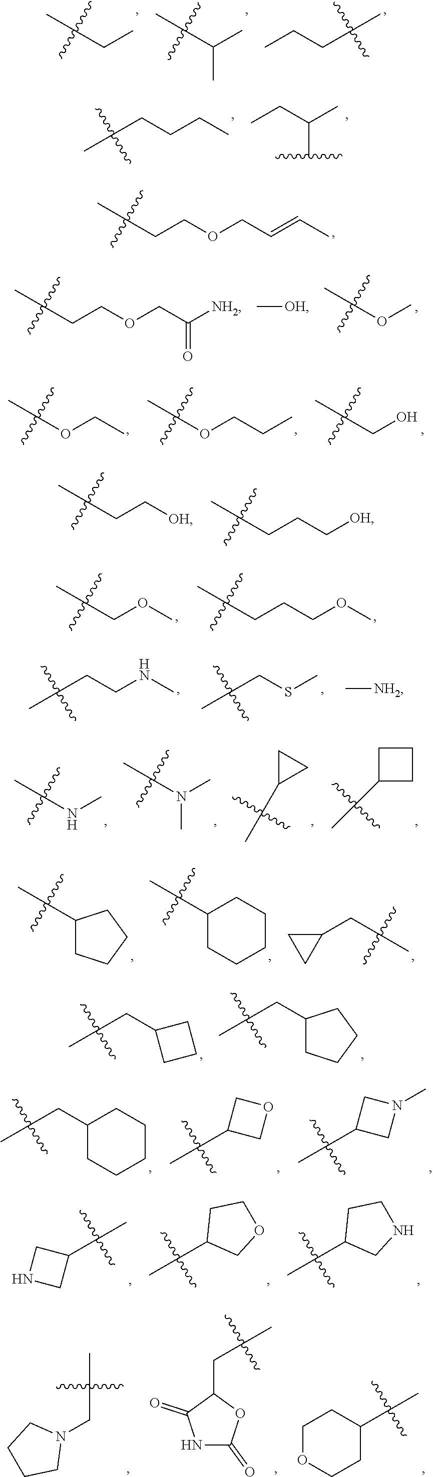

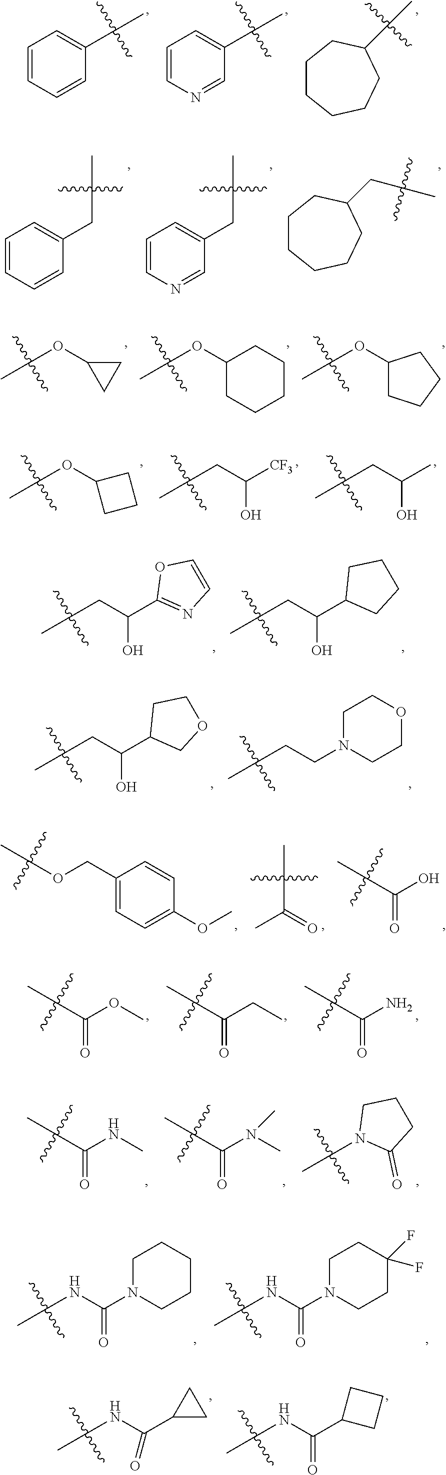

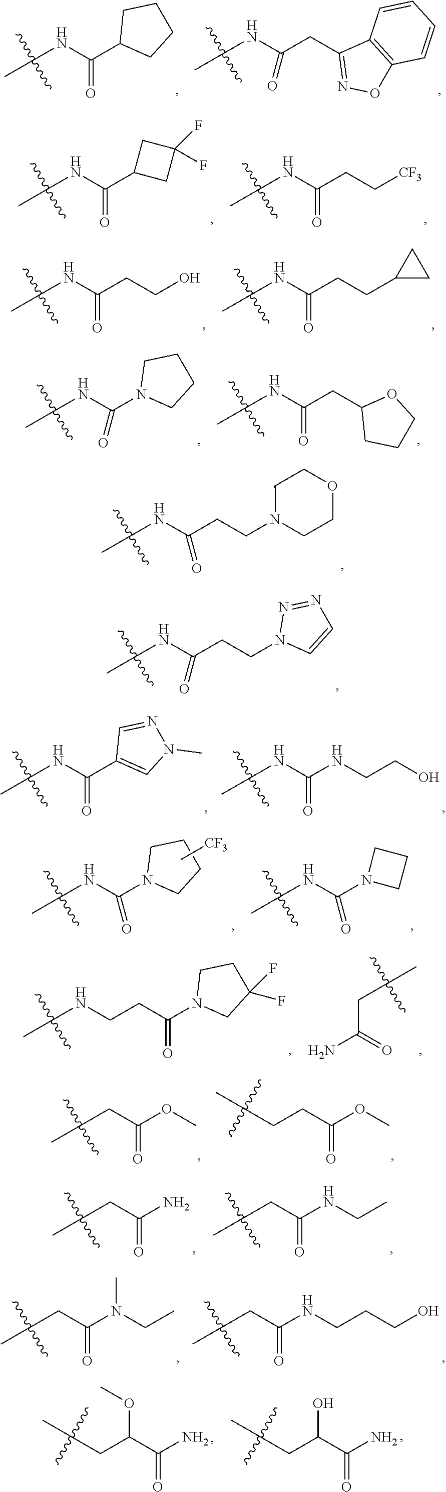

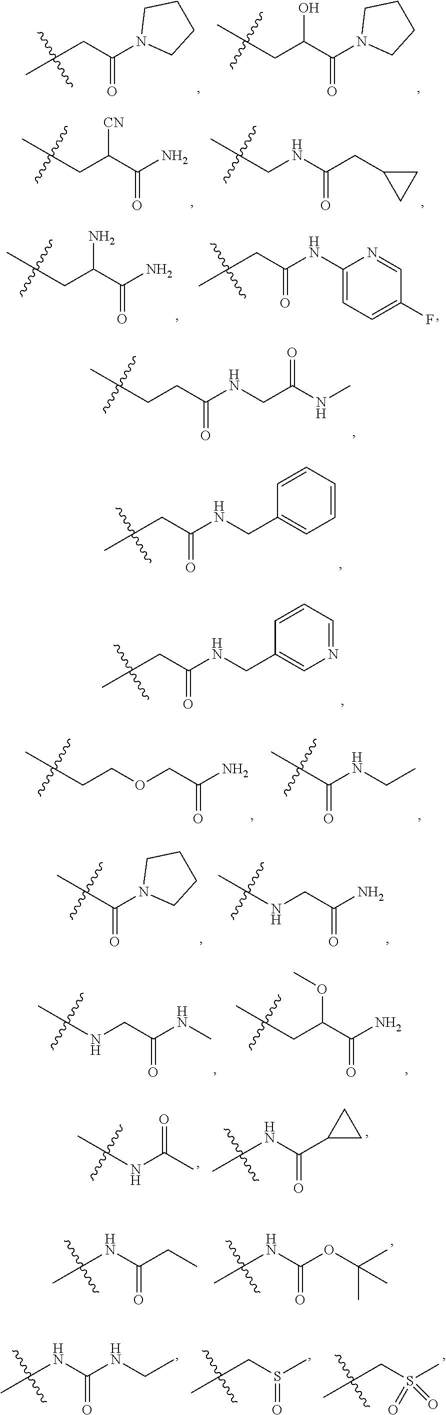

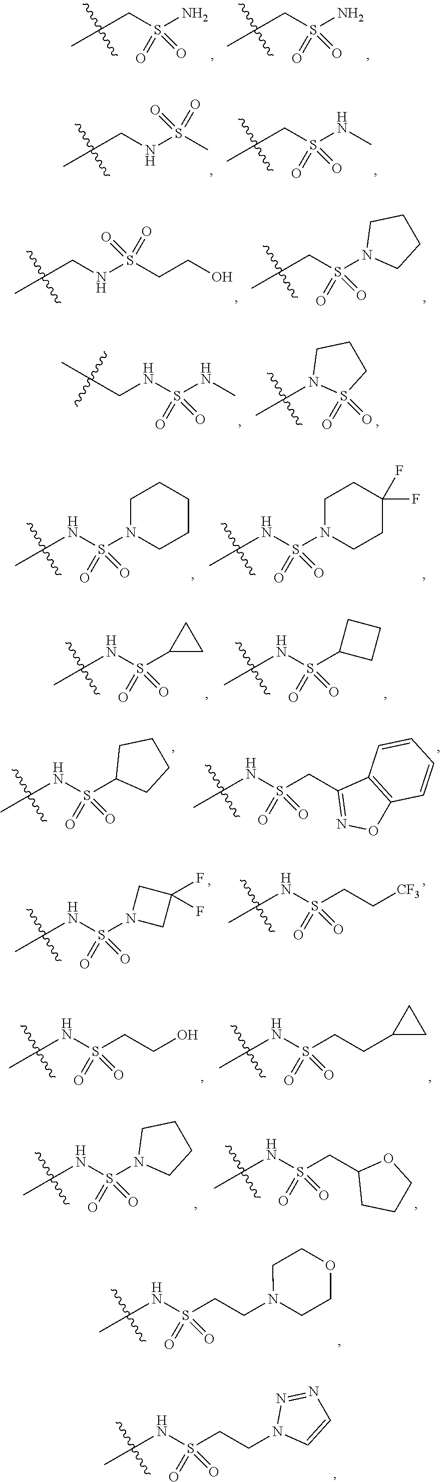

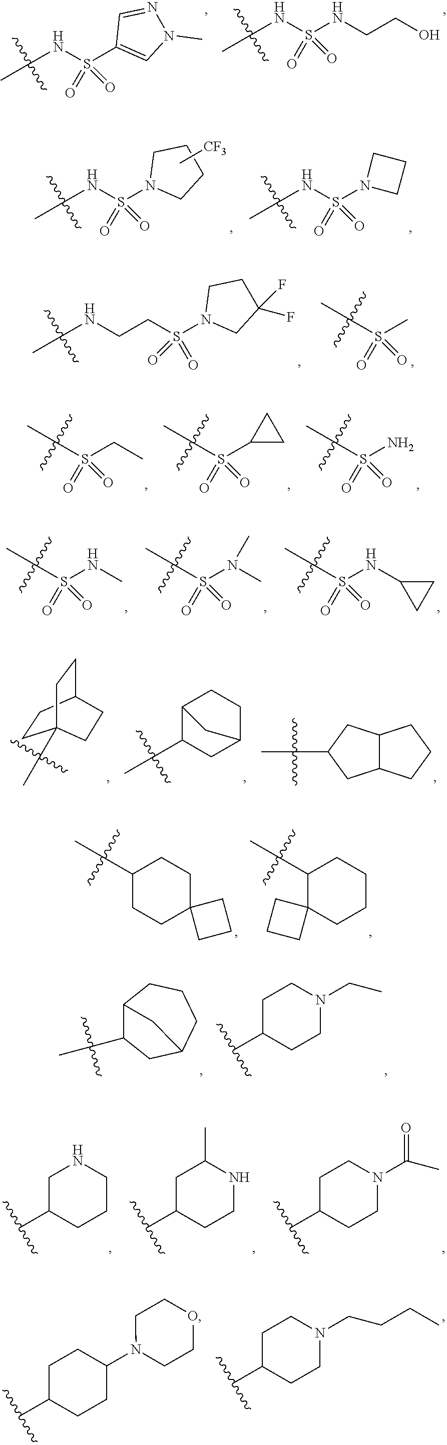

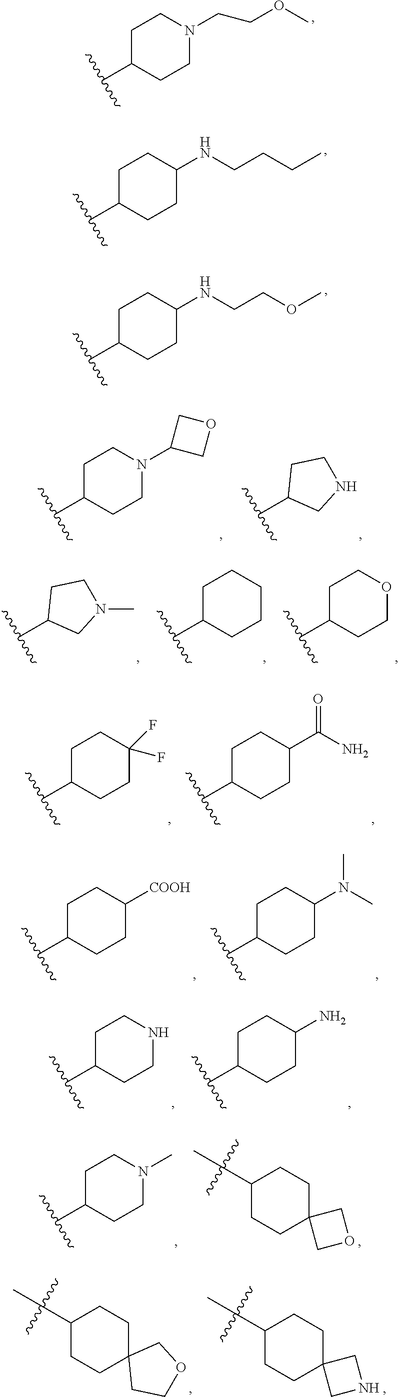

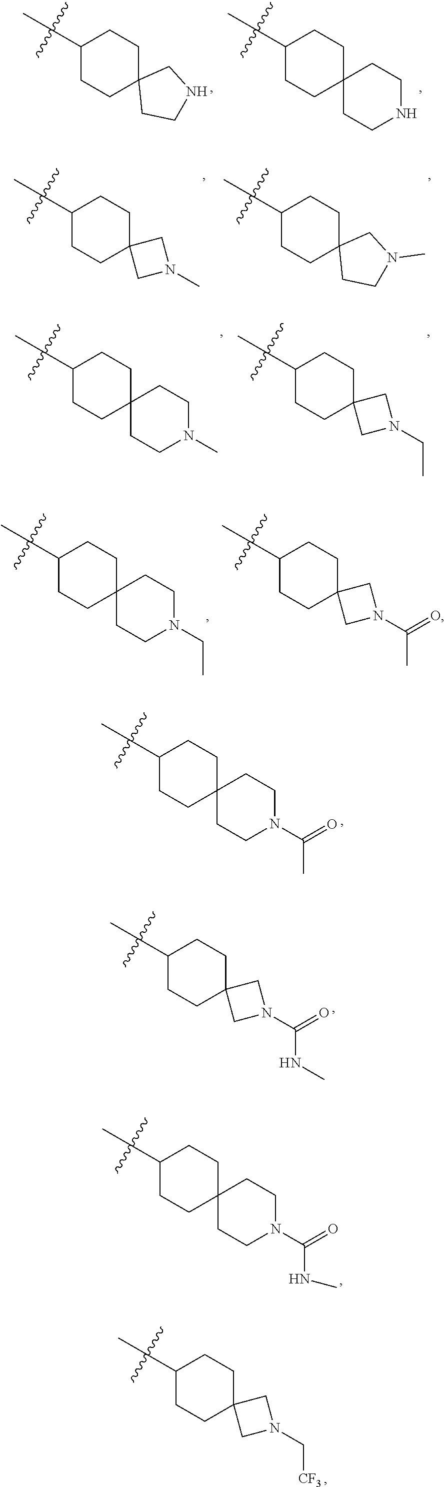

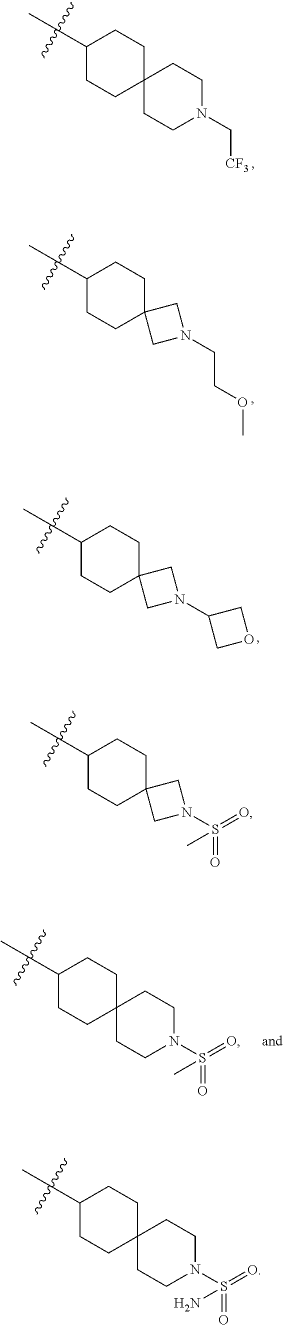 [SMILES: *C1CCC2(CC1)CCN(CC(F)(F)F)CC2 ; *C1CCC2(CC1)CN(CCOC)C2 ; *C1CCC2(CC1)CN(C1COC1)C2 ; *C1CCC2(CC1)CN(S(C)(=O)=O)C2 ; *C1CCC2(CC1)CCN(S(C)(=O)=O)CC2 ; *C1CCC2(CC1)CCN(S(N)(=O)=O)CC2]

More specifically, in formula VIII, R⁶⁰¹ may be selected from: —H, C₁₋₆ alkyl (e.g., methyl, ethyl, n-propyl, or isopropyl), C₃₋₆ cycloalkyl (e.g., cyclopropyl, cyclobutyl, cyclopentyl, or cyclohexyl), heterocycloalkyl

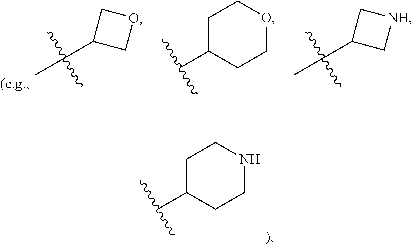 [SMILES: *C1COC1 ; *C1CCOCC1 ; *C1CNC1 ; *C1CCNCC1]

wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, and C₃₋₆ cycloalkyl; further, R⁶⁰¹ may be selected from: —H, and C₁₋₆ alkyl (e.g., methyl, ethyl, n-propyl, or isopropyl), wherein one or more H in the C₁₋₆ alkyl may be substituted with a substituent selected from: halogen (particularly F), —OH, —NH₂, C₁₋₆ alkoxy (e.g., methoxy, or ethoxy), and C₁₋₆ alkylamino

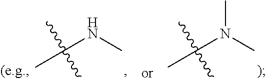

in some embodiments of the present invention, R⁶⁰¹ is selected from: —H, C₁₋₃ alkyl,

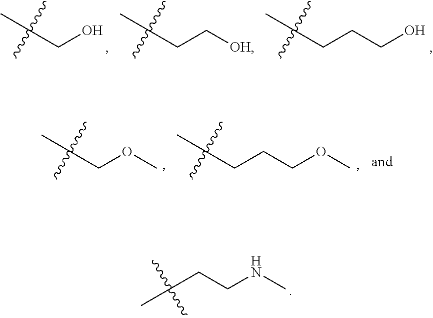

In some embodiments of the present invention, in formula VIII, R⁶⁰² is —H.

In some embodiments of the present invention, in formula VIII, R⁶⁰⁵ is —H.

In some embodiments of the present invention, in formula VIII, R⁶⁰⁶ is —H.

More specifically, in formula VIII, R⁶⁰³ may be selected from: —H, C₁₋₆ alkyl (e.g., methyl, ethyl, n-propyl, or isopropyl), —OH, C₁₋₆ alkoxy (e.g., methoxy, or ethoxy), —NH₂, C₁₋₆ alkylamino (e.g.,

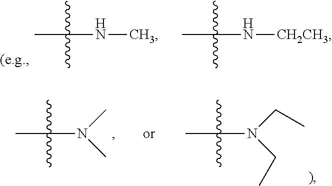

C₃₋₆ cycloalkyl (e.g., cyclopropyl, cyclobutyl, cyclopentyl, or cyclohexyl), heterocycloalkyl

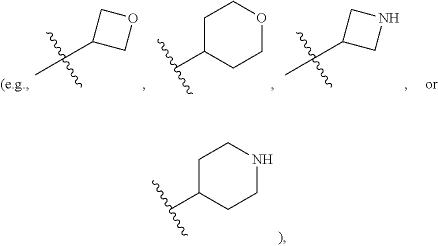 [SMILES: *C1COC1 ; *C1CCOCC1 ; *C1CNC1 ; *C1CCNCC1]

wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, and C₃₋₆ cycloalkyl; further, R⁶⁰³ may be selected from: —H, C₁₋₆ alkoxy (e.g., methoxy, or ethoxy), —NH₂, and C₁₋₆ alkylamino

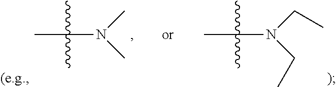

in some embodiments of the present invention, R⁶⁰³ is selected from: —H, methoxy, and

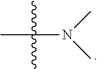 [SMILES: *N(C)C]

More specifically, in formula VIII, R⁶⁰⁴ may be selected from: —H, C₁₋₆ alkyl (e.g., methyl, ethyl, n-propyl, or isopropyl), —OH, C₁₋₆ alkoxy (e.g., methoxy, or ethoxy), —NH₂, C₁₋₆ alkylamino

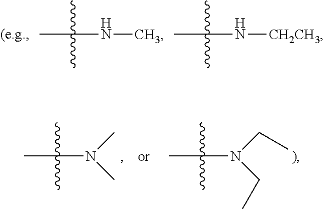

C₃₋₆ cycloalkyl (e.g., cyclopropyl, cyclobutyl, cyclopentyl, or cyclohexyl), heterocycloalkyl

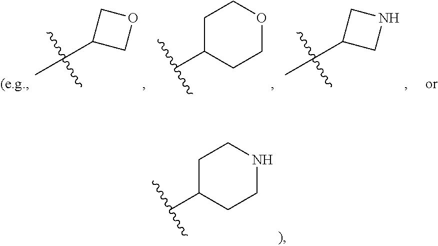 [SMILES: *C1COC1 ; *C1CCOCC1 ; *C1CNC1 ; *C1CCNCC1]

wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, and C₃₋₆ cycloalkyl; further, R⁶⁰⁴ may be selected from: —H, C₁₋₆ alkoxy (e.g., methoxy, or ethoxy), —NH₂, and C₁₋₆ alkylamino

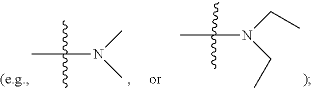

in some embodiments of the present invention, R⁶⁰⁴ is selected from: —H, methoxy, and

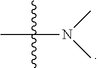 [SMILES: *N(C)C]

In another embodiment of the present invention, ring B is a tricyclic heteroaromatic ring.

Specifically, moiety

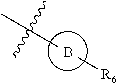

may have the following structure:

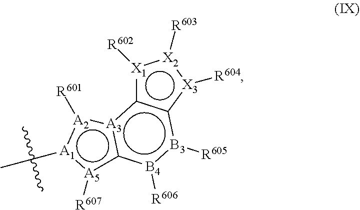

wherein A₁, A₂ and A₃ are independently selected from C, N, O and S; B₁, B₂ and B₃ are independently selected from C and N; B₄ is C, N or absent; X₁, X₂ and X₃ are independently selected from C, N, O and S, and satisfy valence-bond saturation; R⁶⁰¹, R⁶⁰², R⁶⁰³, R⁶⁰⁴, R⁶⁰⁵, R⁶⁰⁶ and R⁶⁰⁷ may be appropriately absent; R⁶⁰¹, R⁶⁰², R⁶⁰³, R⁶⁰⁴, R⁶⁰⁶ and R⁶⁰⁷ are independently selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl, wherein H at the carbon atoms may be substituted with one or more groups selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl.

Specifically, in formula IX, A₁, A₂ and A₃ are independently selected from C and N.

In some embodiments of the present invention, in formula IX, B₁, B₂, B₃ and B₄ are all C; or B₁ is N, and B₂, B₃ and B₄ are all C; or B₂ is N, and B₁, B₃ and B₄ are all C; or B₃ is N, and B₁, B₂ and B₄ are all C; or B₄ is N, and B₂, B₃ and B₁ are all C.

Specifically, in formula IX, X₁, X₂ and X₃ are independently selected from C and N.

In some embodiments of the present invention, in formula IX, X₁ is C, X₂ is C, and X₃ is N.

More specifically, moiety

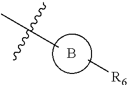

may be selected from the following structures:

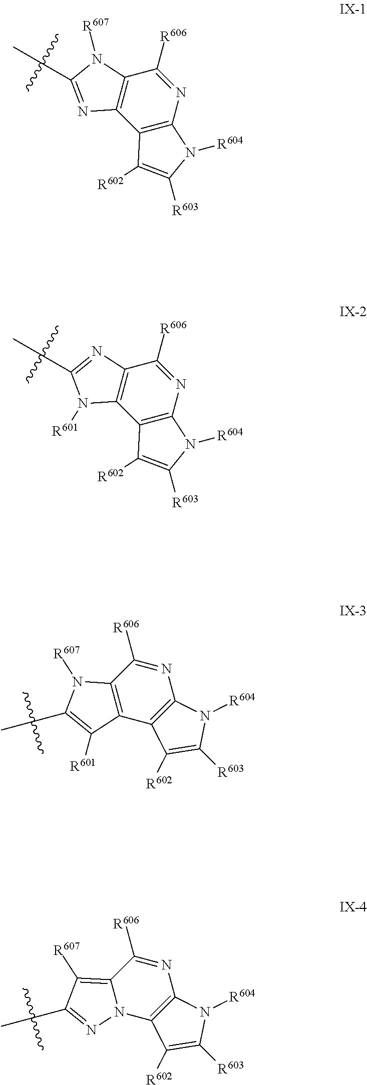

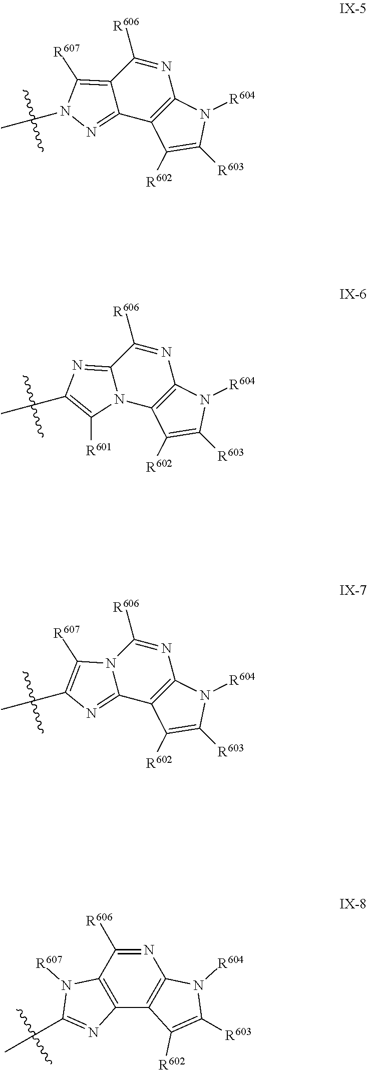

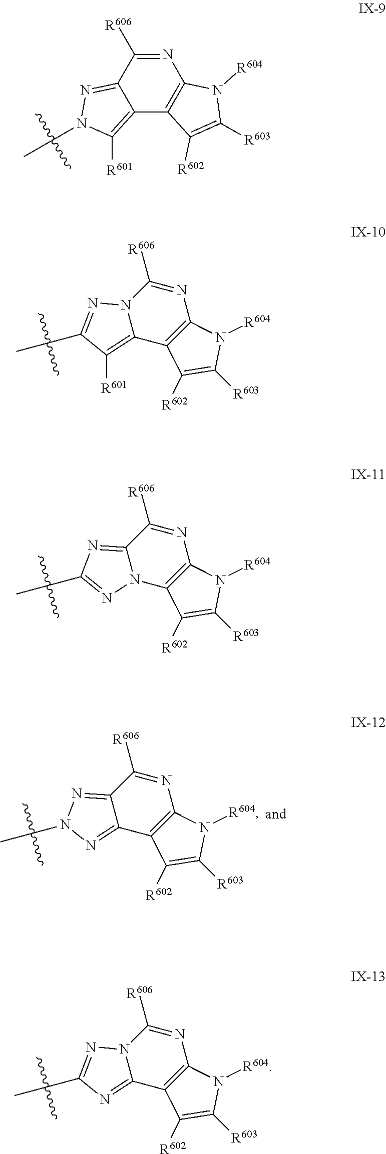

More specifically, in formula IX, R⁶⁰¹, R⁶⁰², R⁶⁰³, R⁶⁰⁴, R⁶⁰⁵, R⁶⁰⁶ and R⁶⁰⁷ are independently selected from: —H, -D, —F, —Cl, —Br, —I, —CF₃, —CHF₂, —CH₂F, —CH₂CF₃, —CN, —NO₂, —CH₃,

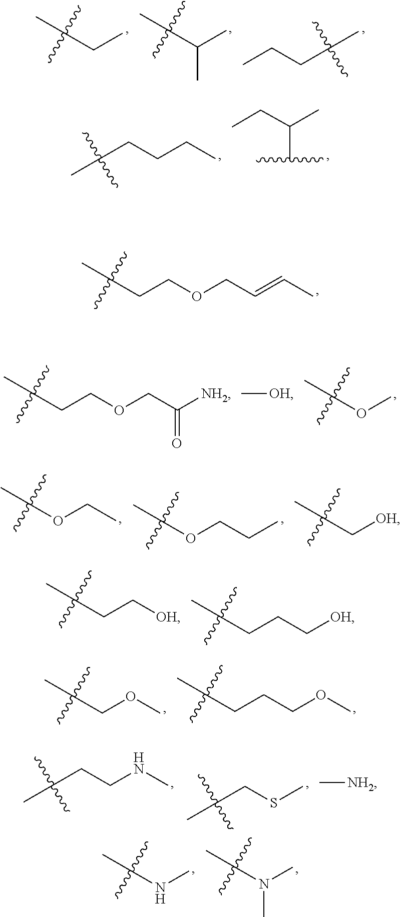

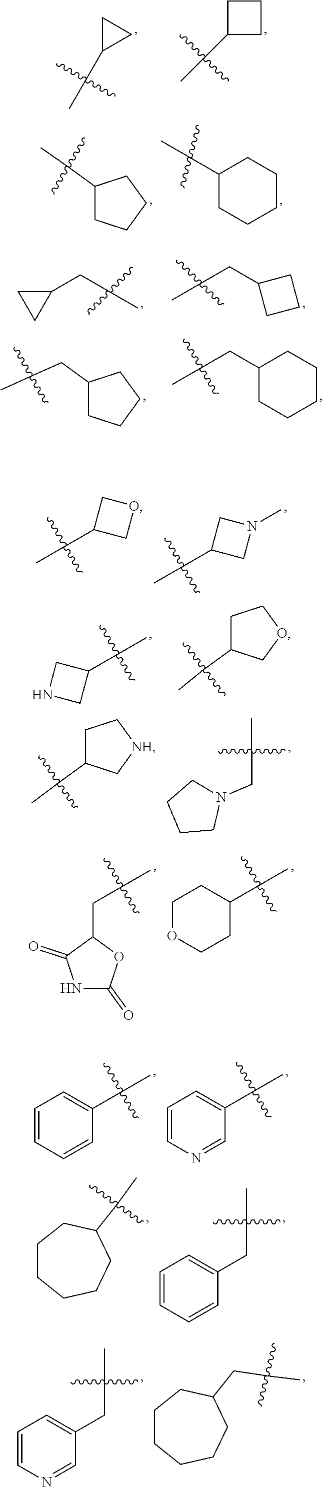

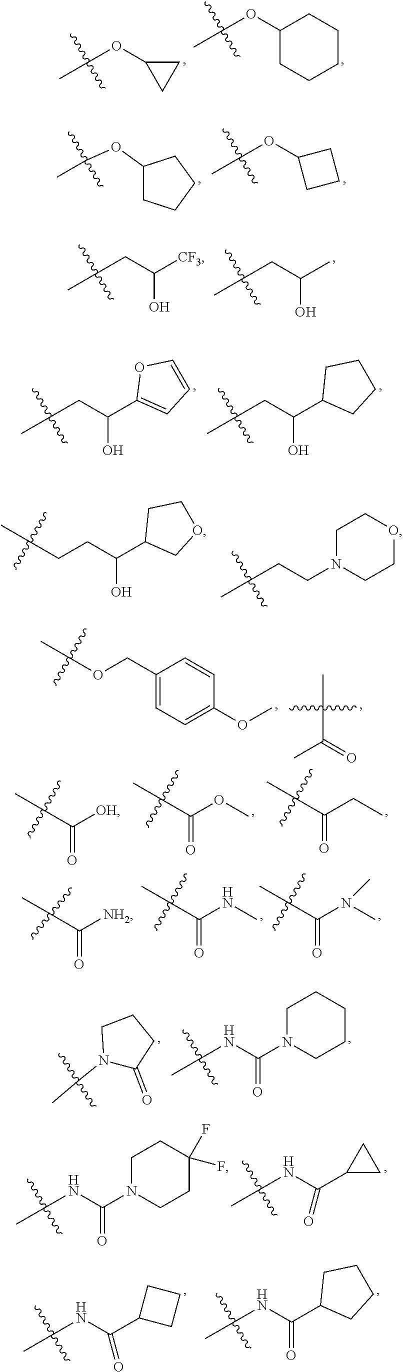

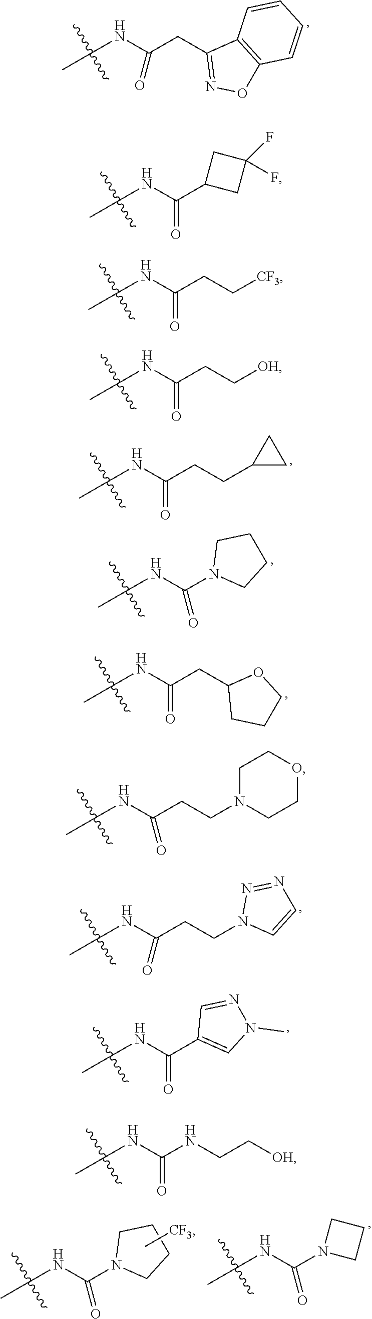

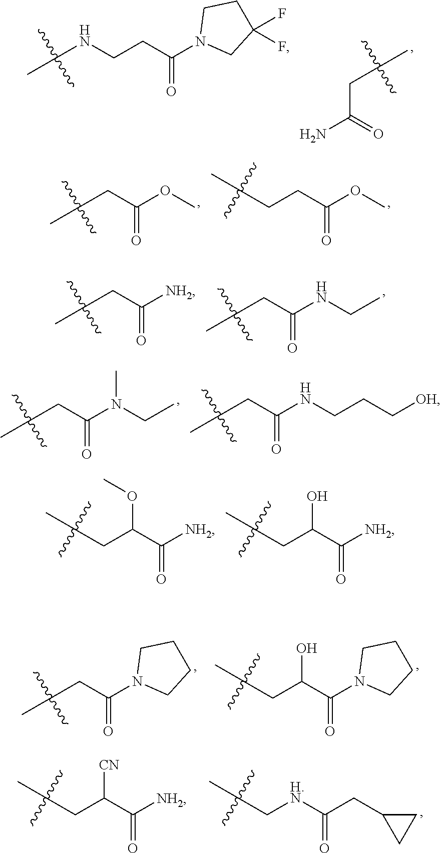

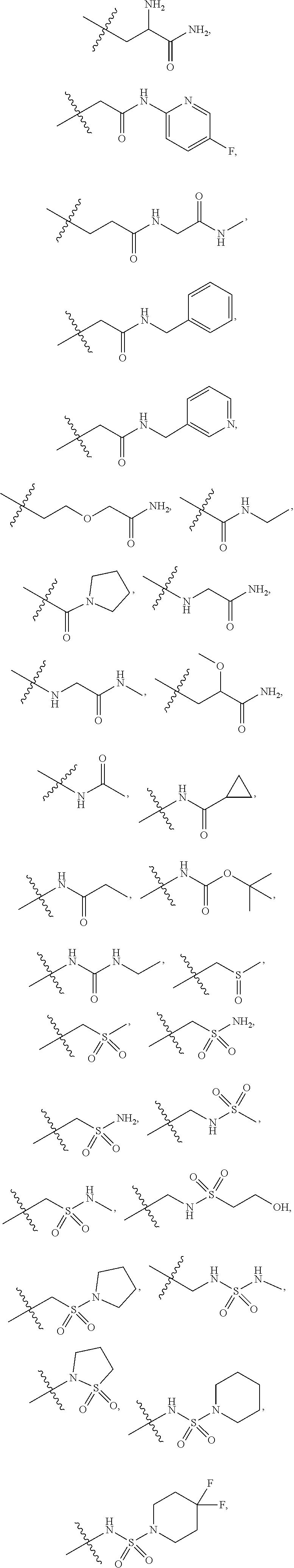

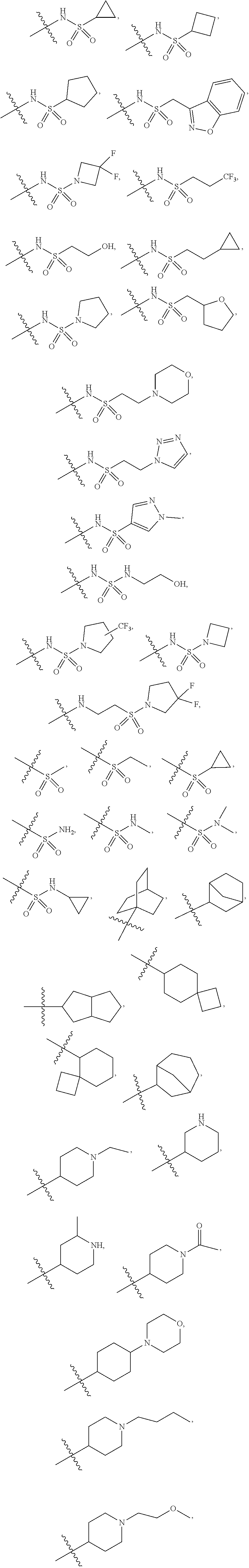

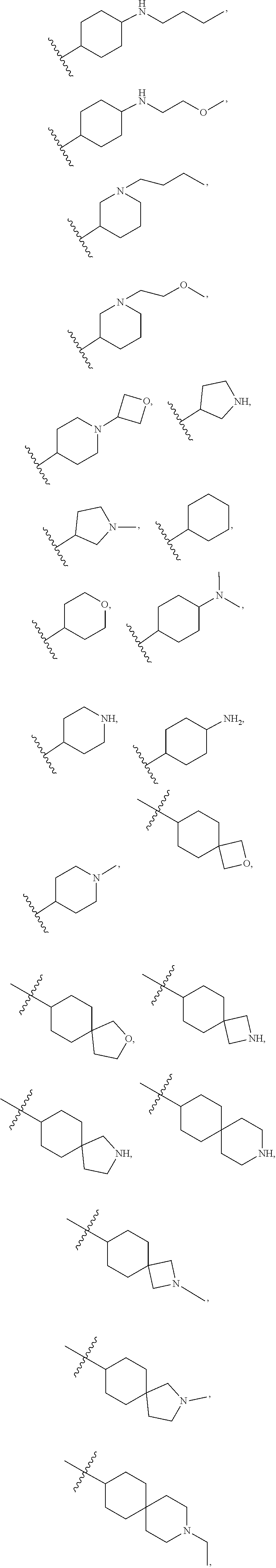

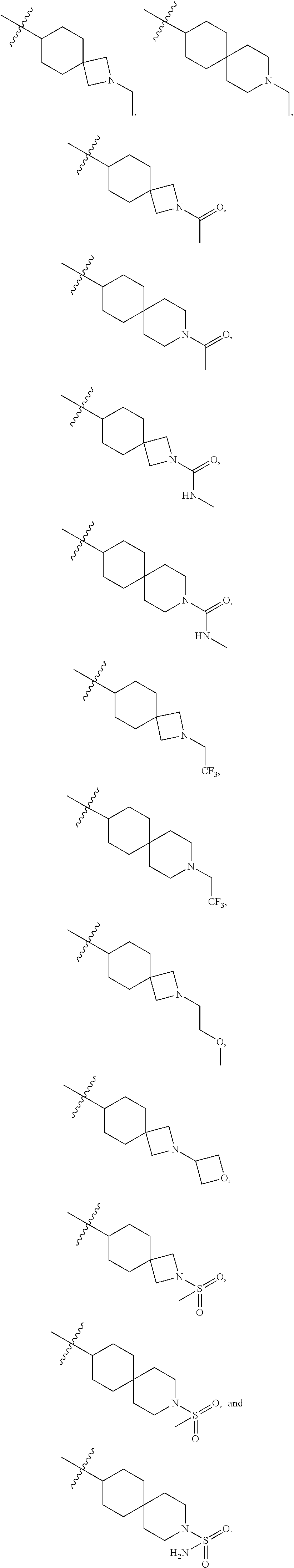

In some embodiments of the present invention, in formula IX, R⁶⁰¹ is selected from: —H, C₁₋₆ alkyl (e.g., methyl, ethyl, n-propyl, or isopropyl), and C₃₋₆ cycloalkyl (e.g. cyclopropyl, cyclobutyl, cyclopentyl, or cyclohexyl).

In some embodiments of the present invention, in formula IX, R⁶⁰³ is —H.

In some embodiments of the present invention, in formula IX, R⁶⁰⁴ is selected from: —H, C₁₋₆ alkyl (e.g., methyl, ethyl, n-propyl, or isopropyl), and C₃₋₆ cycloalkyl (e.g. cyclopropyl, cyclobutyl, cyclopentyl, or cyclohexyl).

In some embodiments of the present invention, in formula IX, R⁶⁰⁶ is —H.

In some embodiments of the present invention, in formula IX, R⁶⁰⁷ is selected from: —H, C₁₋₆ alkyl (e.g., methyl, ethyl, n-propyl, or isopropyl), and C₃₋₆ cycloalkyl (e.g. cyclopropyl, cyclobutyl, cyclopentyl, or cyclohexyl).

In some embodiments of the present invention, in formula IX, R⁶⁰² is selected from:

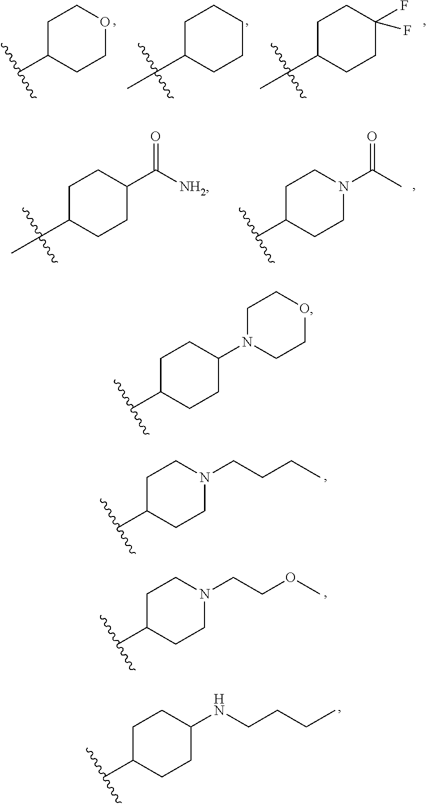

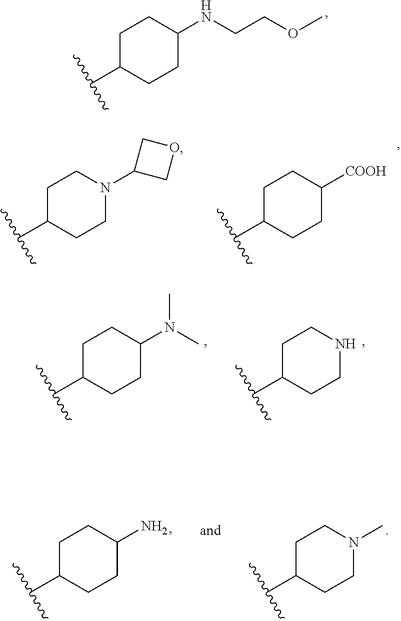

In some embodiments of the present invention,

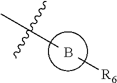

may be selected from:

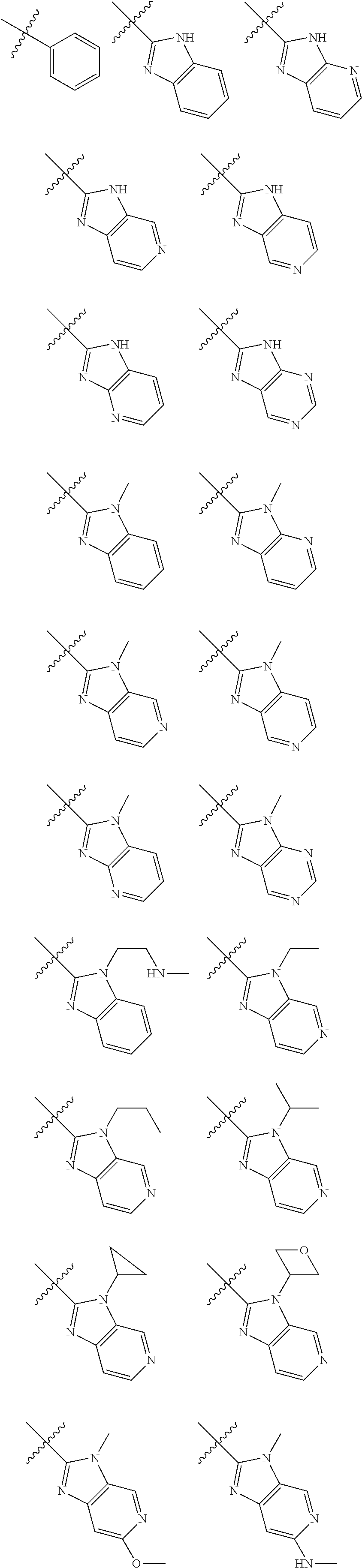

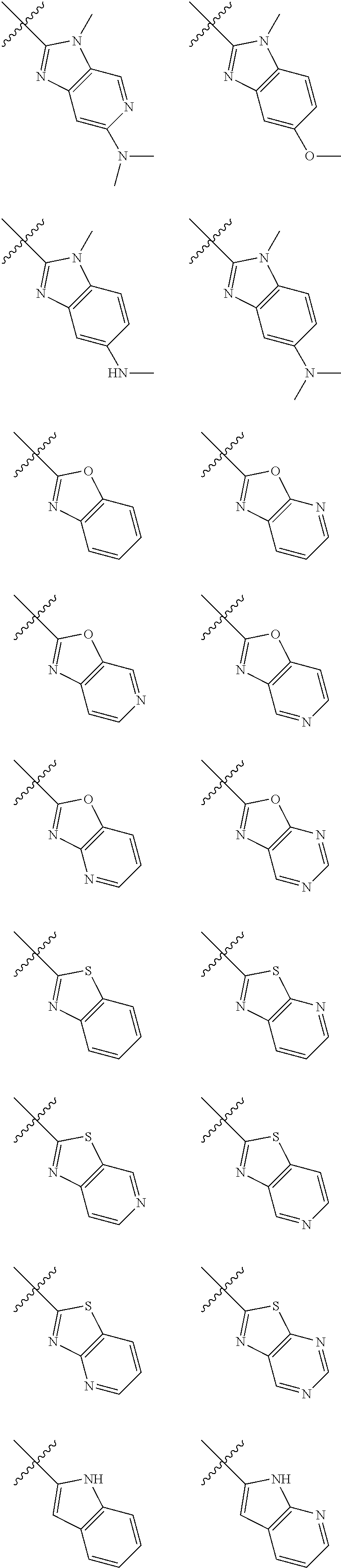

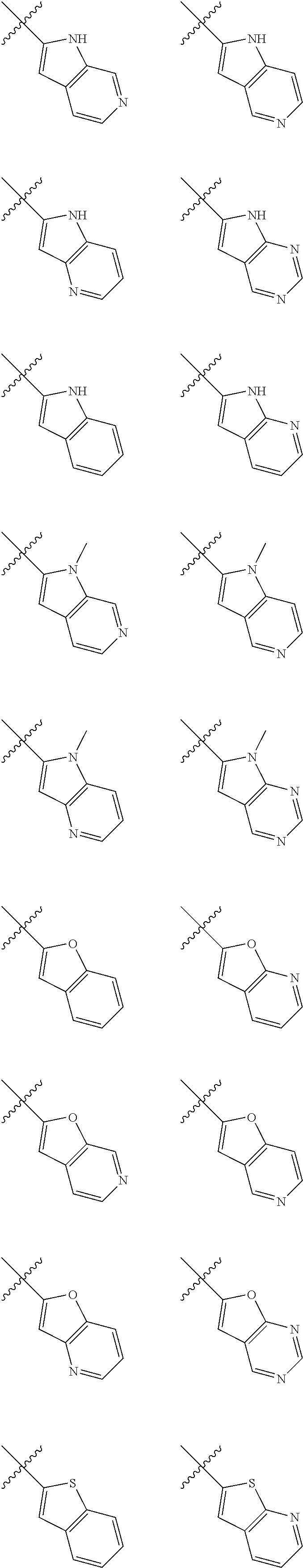

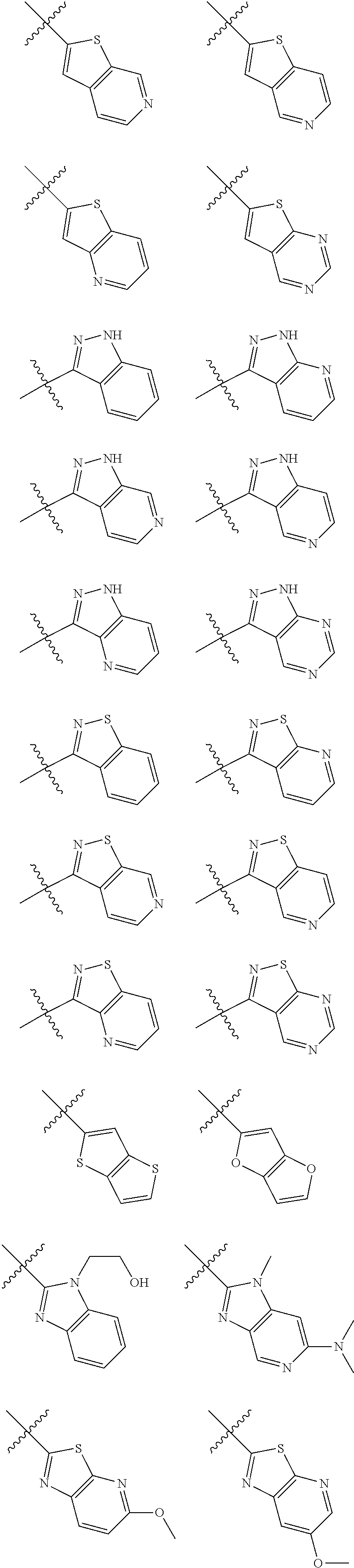

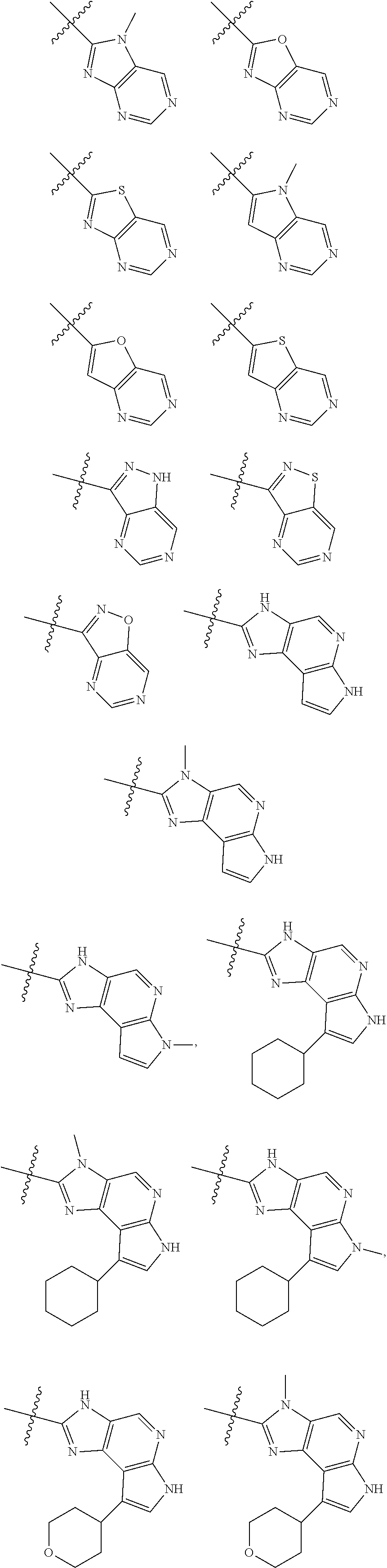

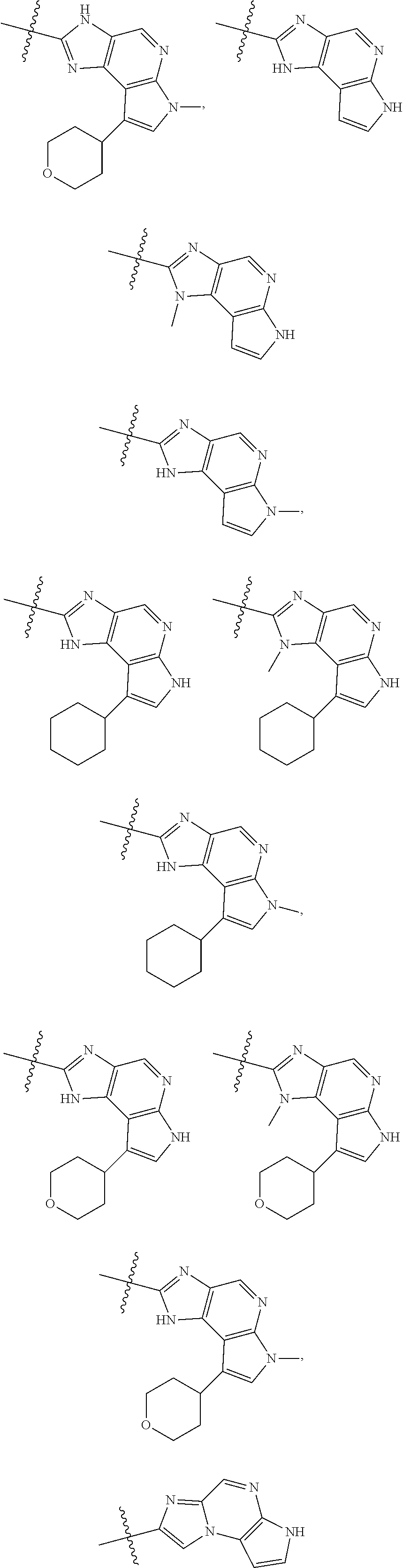

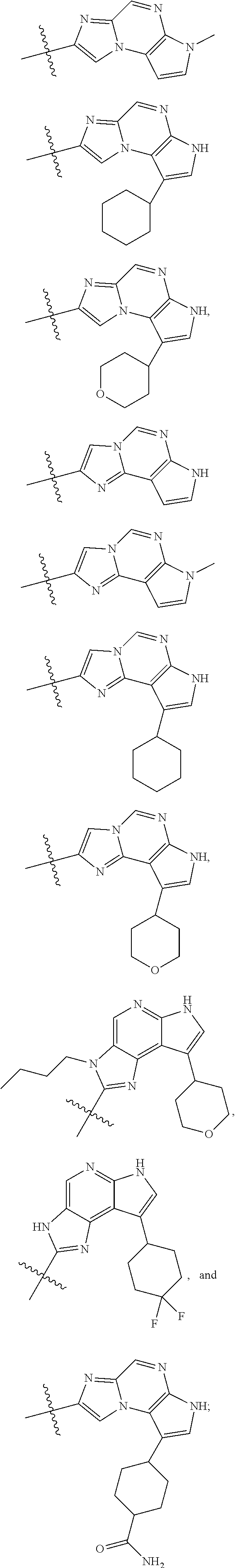

wherein H linked to carbon atoms may be optionally substituted with one or more groups selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl.

Specifically, R₁ may be selected from: H, halogen, —CN, —NO₂, —CF₃, —OCF₃, C₁₋₆ alkyl, —O(C₁₋₆ alkyl), —N(C₁₋₆ alkyl)(C₁₋₆ alkyl), —COOH, —COO(C₁₋₆ alkyl), —OCOH, —OCO(C₁₋₆ alkyl), —CONH(C₁₋₆ alkyl), —CON(C₁₋₆ alkyl)(C₁₋₆ alkyl),

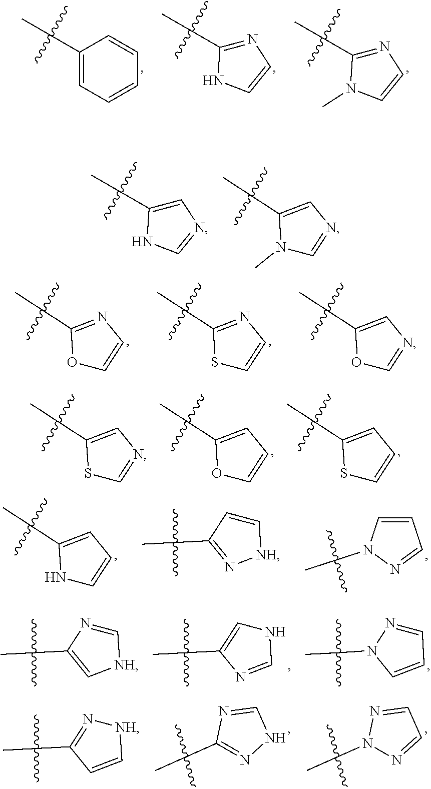

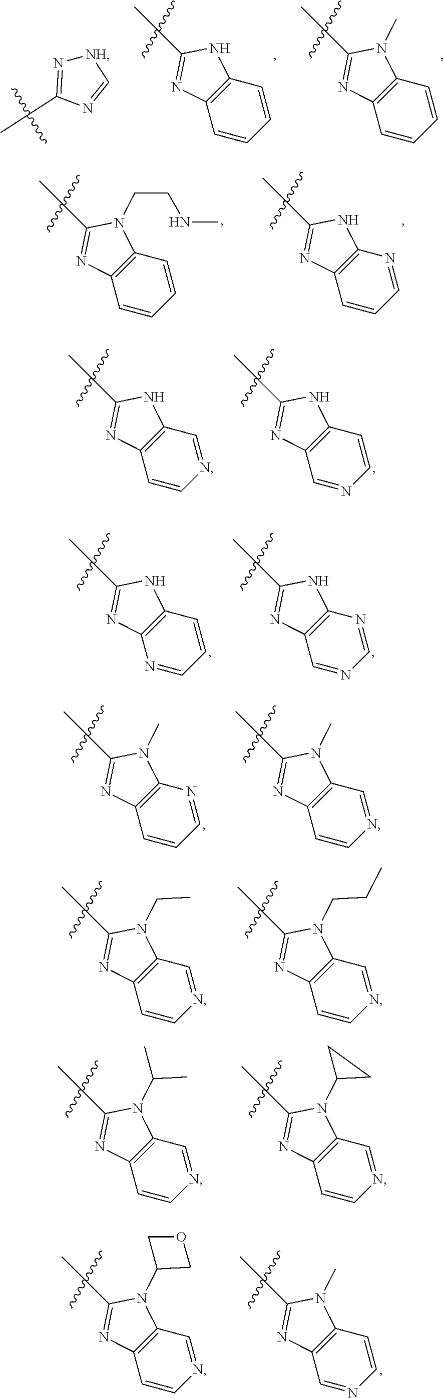

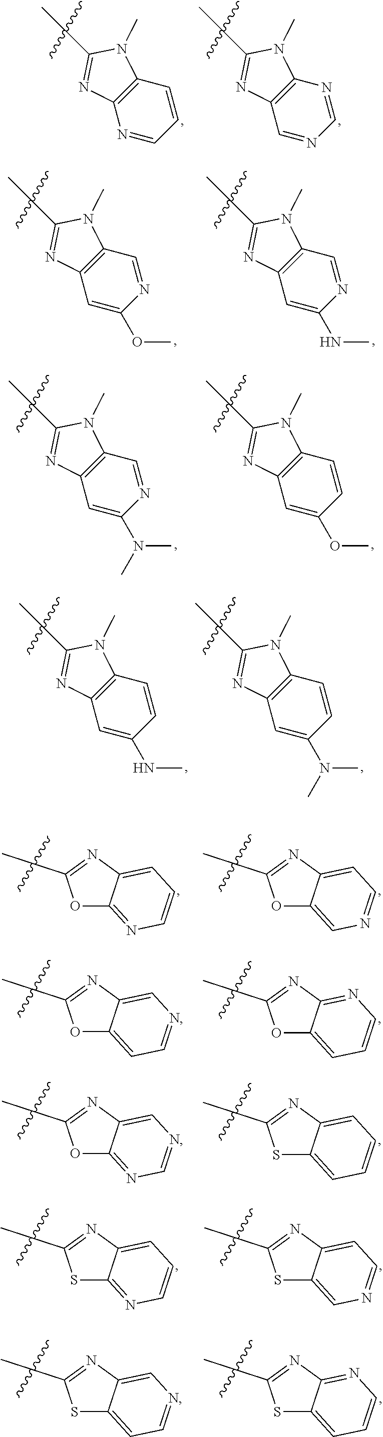

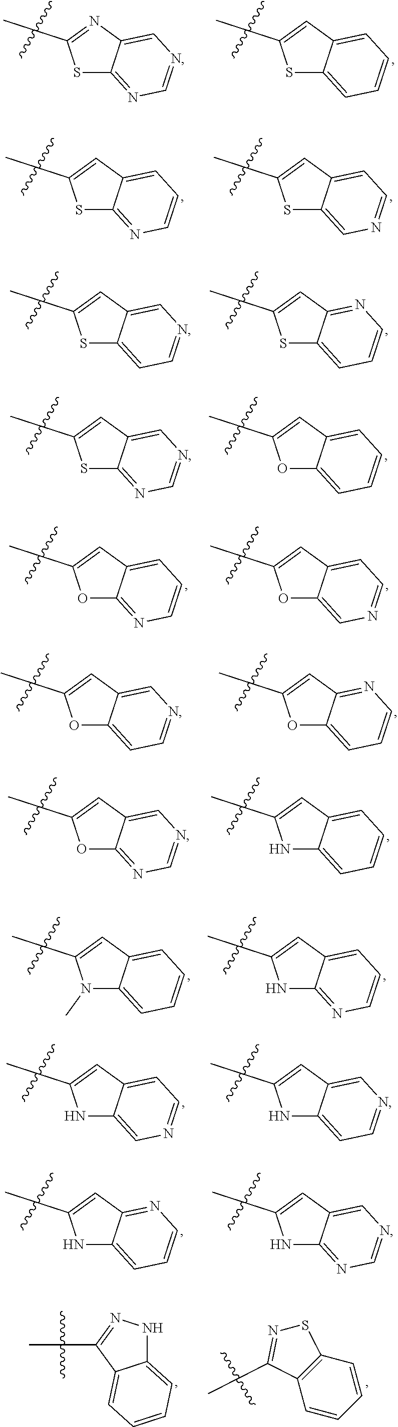

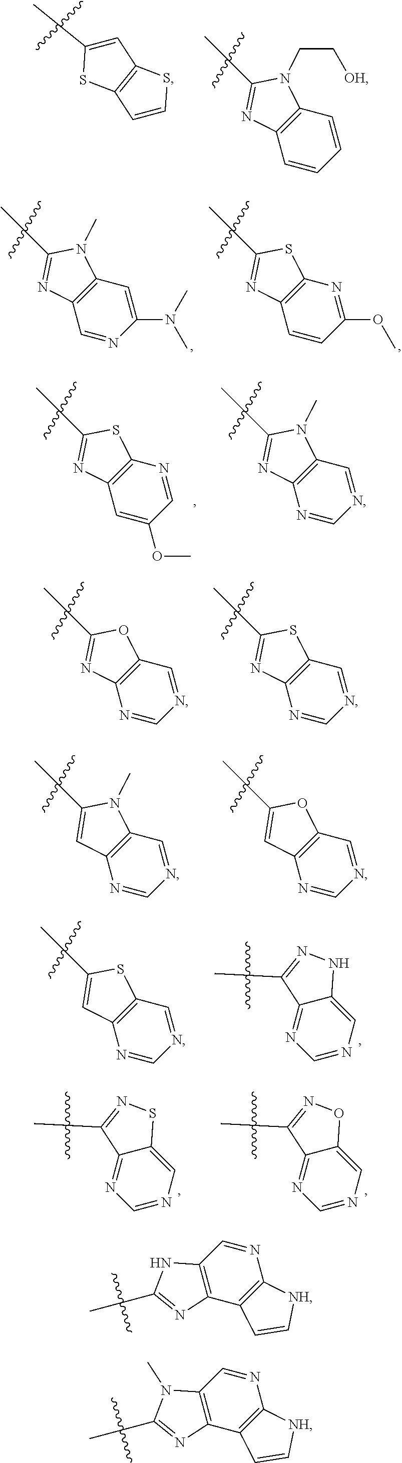

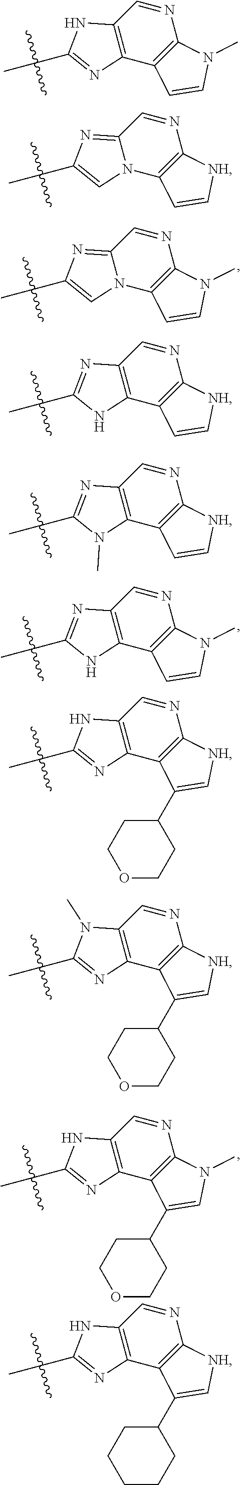

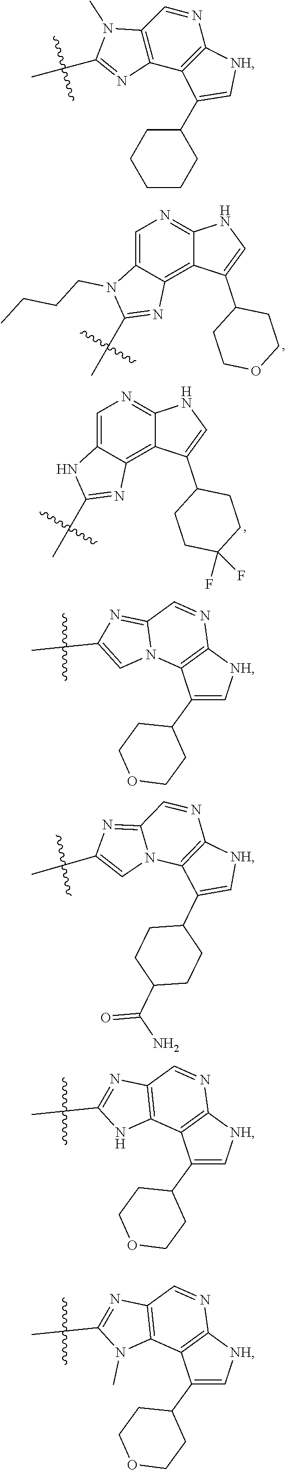

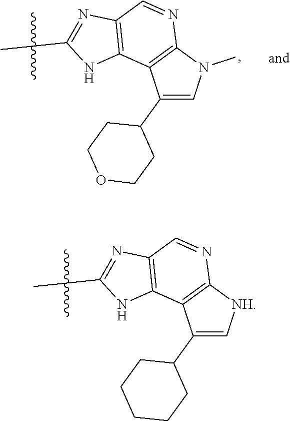 [SMILES: *c1nc2cnc3c(c(C4CCOCC4)cn3C)c2[nH]1 ; *c1nc2cnc3[nH]cc(C4CCCCC4)c3c2[nH]1]

In some embodiments, the present invention provides the following enumerated embodiments 1-56:

Embodiment 1. A compound of Formula A, or a pharmaceutically acceptable salt thereof,

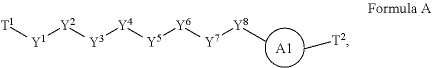

wherein:
T¹ is a glutarimide containing moiety;
T² is hydrogen or an optionally substituted fused 8-14 membered bicyclic or tricyclic heteroaryl having 1-6 ring heteroatoms each independently selected from N, S, and O, preferably, T² is not hydrogen;
Ring A1 is a 4-10 membered monocyclic or bicyclic carbocyclic or heterocyclic ring, which is optionally substituted with one or more substituents each independently halogen, OH, CN, oxo, NH₂, C₁₋₆ alkyl, C₂_₆ alkenyl, C₂₋₆ alkynyl, C₁₋₆ heteroalkyl, or a 3-7 membered ring selected from C₃_₇ carbocyclic, 3-7 membered heterocyclic, phenyl, or 5 or 6 membered heteroaryl, wherein the C₁₋₆ alkyl, C₂₋₆ alkenyl, C₂₋₆ alkynyl, C₁₋₆ heteroalkyl, or 3-7 membered ring is optionally substituted;
Y¹ to Y⁷ are defined according to (1)-(11) below:

Y¹ is NH,O,S,S(O),SO₂,C(G^{A})(G^{B}),C(O), or N(G^{C}); Y² is NH,A,S,S(O),SO₂,C(G^{A})(G^{B}),C(O), or N(G^{C}) Y³ is NH,O,S,S(O),SO₂,C(G^{A})(G^{B}),C(O), or N(G^{C}); Y⁴ is NH,O,S,S(O),SO₂,C(G^{A})(G^{B}),C(O), or N(G^{C}); Y⁵ is NH,O,S,S(O),SO₂,C(G^{A})(G^{B}),C(O), or N(G^{C}); Y⁶ is NH,O,S,S(O),SO₂,C(G^{A})(G^{B}),C(O), or N(G^{C}); Y⁷ is NH,O,S,S(O),SO₂,C(G^{A})(G^{B}),C(O), or N(G^{C}); (1)
wherein each of G^{A} and G^{B} at each occurrence is independently hydrogen, deuterium, halogen, OH, C₁₋₄ alkyl optionally substituted with halogen and/or OH, or C₁₋₄ heteroalkyl optionally substituted with halogen and/or OH, or two adjacent G^{A} are joined to form a double bond, or two adjacent C(G^{A})(G^{B}) represent a triple bond, or G^{A} and G^{B} together with the carbon atom they are both attached to are joined to form an optionally substituted 3-7 membered carbocyclic or heterocyclic ring; G^{C} at each occurrence is independently an optionally substituted group selected from C₁₋₄ alkyl, C₁₋₄ heteroalkyl, or a 3-8 membered ring;
provided that:
   (i) two adjacent groups of Y¹ to Y⁷ are not both selected from NH, O, S, or N(G^{C});
   (ii) two adjacent groups of Y¹ to Y⁷ are not both selected from S(O), SO₂, or C(O);
   (iii) at most three groups selected from Y¹ to Y⁷ can be S(O), SO₂, or C(O);
   (iv) at most two groups selected from Y¹ to Y⁷ can be C(G^{A})(G^{B}) wherein G^{A} and G^{B} together with the carbon atom they are both attached to are joined to form an optionally substituted 3-6 membered carbocyclic or heterocyclic ring; and
   (v) the combination of Y¹ to Y⁷ does not contain a bond selected from S—S(O), S—SO₂, and S—C(O);
(2) Y³, Y⁴, and Y⁵ together represent an optionally substituted 3-8 membered ring, and Y¹, Y², Y⁶, and Y⁷ are as defined in (1);
(3) Y⁴, Y⁵, and Y⁶ together represent an optionally substituted 3-8 membered ring, and Y¹, Y², Y³, and Y⁷ are as defined in (1);
(4) Y⁵, Y⁶, and Y⁷ together represent an optionally substituted 3-8 membered ring, and Y¹, Y², Y³, and Y⁴ are as defined in (1);
(5) Y³, Y⁴, Y⁵, and Y⁶ together represent an optionally substituted 3-10 membered ring, and Y¹, Y², and Y⁷ are as defined in (1);
(6) Y⁴, Y⁵, Y⁶, and Y⁷ together represent an optionally substituted 3-10 membered ring, and Y¹, Y², and Y³ are as defined in (1);
(7) Y², Y³, and Y⁴ together represent an optionally substituted 3-8 membered ring, and Y¹, Y⁵, Y⁶, and Y⁷ are as defined in (1);
(8) Y², Y³, Y⁴, and Y⁵ together represent an optionally substituted 3-10 membered ring, and Y⁵, Y⁶, and Y⁷ are as defined in (1);
(9) Y², Y³, Y⁴, Y⁵, and Y⁶ together represent an optionally substituted 3-10 membered ring, and Y¹ and Y⁷ are as defined in (1);
(10) Y³, Y⁴, Y⁵, Y⁶, and Y⁷ together represent an optionally substituted 3-10 membered ring, preferably, 8-10 membered heterocyclic or heteroaryl ring, and Y¹ and Y² are as defined in (1);
(11) Y², Y³, Y⁴, Y⁵, Y⁶ and Y⁷ together represent an optionally substituted 3-10 membered ring, preferably, 8-10 membered heterocyclic or heteroaryl ring, and Y¹ is as defined in (1); and
Y⁸ is null, O, NH, C(O), an optionally substituted C₁₋₆ alkylene, or an optionally substituted C₁₋₆ heteroalkylene.

Embodiment 2. The compound of Embodiment 1, or a pharmaceutically acceptable salt thereof, wherein T¹ is a moiety having a structure according to

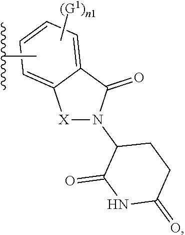

wherein:
X is CH₂ or C(═O),
n1 is an integer of 0-2, and
G¹ at each occurrence is independently halogen, CN, OH, NH₂, an optionally substituted C₁₋₆ alkyl, optionally substituted C₁₋₆ heteroalkyl, optionally substituted 3-8 membered carbocyclic or heterocyclic ring, optionally substituted phenyl, or optionally substituted heteroaryl.

Embodiment 3. The compound of Embodiment 2, or a pharmaceutically acceptable salt thereof, characterized as having a structure according to Formula A1:

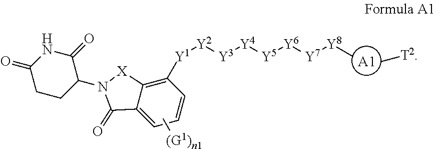

Embodiment 4. The compound of Embodiment 2 or 3, or a pharmaceutically acceptable salt thereof, wherein n1 is 0.

Embodiment 5. The compound of any of Embodiments 1-4, or a pharmaceutically acceptable salt thereof, wherein Y¹ is NH, O, or N(G^{C}), preferably, Y¹ is NH or O.

Embodiment 6. The compound of any of Embodiments 1-5, or a pharmaceutically acceptable salt thereof, wherein Y² is C(G^{A})(G^{B}), preferably, each of G^{A} and G^{B} is independently hydrogen, deuterium, halogen, or C₁₋₄ alkyl (e.g., methyl), or G^{A} and G^{B} together with the carbon atom they are both attached to are joined to form a 3-6 membered carbocyclic or heterocyclic ring, which is optionally substituted with one or more substituents each independently selected from halogen, OH, and C₁₋₄ alkyl (e.g., methyl), for example, Y² is CH₂, CH(CH₃), C(CH₃)₂,

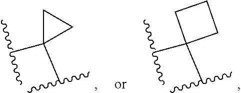 [SMILES: *C1(*)CC1 ; *C1(*)CCC1]

preferably, Y¹-Y² is —NHCH₂— or —OCH₂—.

Embodiment 7. The compound of any of Embodiments 1-6, or a pharmaceutically acceptable salt thereof, wherein Y³, Y⁴, and Y⁵ together represent an optionally substituted 3-8 membered ring selected from 3-8 membered carbocyclic ring, 3-8 membered heterocyclic ring, phenyl ring, or 5- or 6-membered heteroaryl ring, preferably, Y³, Y⁴, and Y⁵ together represent an optionally substituted phenylene or an optionally substituted 5 or 6-membered heteroarylene.

Embodiment 8. The compound of any of Embodiments 1-6, or a pharmaceutically acceptable salt thereof, wherein Y³, Y⁴, and Y⁵ together represent an optionally substituted phenylene or an optionally substituted 5 or 6-membered heteroarylene, wherein Y² and Y⁶ are not ortho to each other on the phenyl or heteroaryl ring, preferably, Y² and Y⁶ are meta to each other.

Embodiment 9. The compound of any of Embodiments 1-6, or a pharmaceutically acceptable salt thereof, wherein Y³, Y⁴, and Y⁵ together represent a 1,3-phenylene, which is optionally substituted with one or more substituents each independently selected from halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁. 4 alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

Embodiment 10. The compound of any of Embodiments 1-6, or a pharmaceutically acceptable salt thereof, wherein Y³, Y⁴, and Y⁵ together represent a 5 or 6-membered heteroarylene, such as a 2,5-thiophenylene

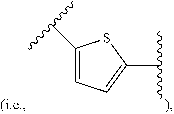 [SMILES: *c1ccc(*)s1]

2,5-furanylene, 2,4-pyridnylene, 2,6-pyridnylene, 3,5-pyridnylene, 2,4-pyrimidinylene, 2,6-pyrimidinylene, or 4,6-pyrimidinylene, etc., wherein the 5 or 6-membered heteroarylene is optionally substituted with one or more substituents each independently selected from halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁-4 alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2} or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

Embodiment 11. The compound of any of Embodiments 1-6, or a pharmaceutically acceptable salt thereof, wherein Y³, Y⁴, and Y⁵ together represent a 3-6 membered carbocyclic or heterocyclic ring, which is optionally substituted with one or more substituents each independently oxo, halogen, OH, NH₂, C₁₋₄ alkyl optionally substituted with F, C₁₋₄ alkoxy optionally substituted with F, or a 3-4 membered carbocyclic or heterocyclic ring optionally substituted with F and/or methyl, for example, Y³, Y⁴, and Y⁵ together represent

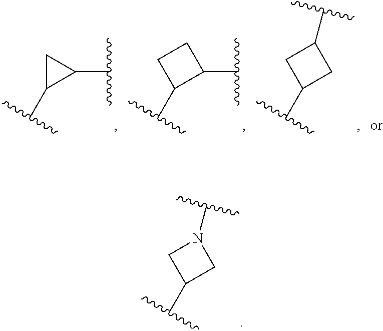 [SMILES: *C1CC1* ; *C1CCC1* ; *C1CC(*)C1 ; *C1CN(*)C1]

Embodiment 12. The compound of any of Embodiments 1-6, or a pharmaceutically acceptable salt thereof, wherein Y³ is C(G^{A})(G^{B}), preferably, each of G^{A} and G^{B} is independently hydrogen, deuterium, halogen, or C₁₋₄ alkyl (e.g., methyl), or G^{A} and G^{B} together with the carbon atom they are both attached to are joined to form a 3-6 membered carbocyclic or heterocyclic ring, which is optionally substituted with one or more substituents each independently selected from halogen, OH, and C₁₋₄ alkyl (e.g., methyl), for example, Y³ is CH₂,

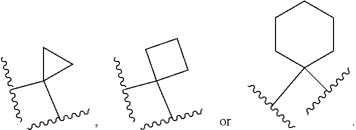 [SMILES: *C1(*)CC1 ; *C1(*)CCC1 ; *C1(*)CCCCC1]

Embodiment 13. The compound of any of Embodiments 1-6 and 12, or a pharmaceutically acceptable salt thereof, wherein Y⁴ is O, NH, N(C₁₋₄ alkyl), or C(G^{A})(G^{B}), preferably, each of G^{A} and G^{B} is independently hydrogen, deuterium, halogen, or C₁₋₄ alkyl (e.g., methyl), or G^{A} and G^{B} together with the carbon atom they are both attached to are joined to form a 3-6 membered carbocyclic or heterocyclic ring, which is optionally substituted with one or more substituents each independently selected from halogen, OH, and C₁₋₄ alkyl (e.g., methyl), for example, Y⁴ is O, CH₂, CH(CH₃), C(CH₃)₂,

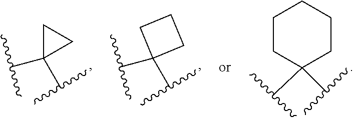 [SMILES: *C1(*)CC1 ; *C1(*)CCC1 ; *C1(*)CCCCC1]

Embodiment 14. The compound of any of Embodiments 1-6 and 12-13, or a pharmaceutically acceptable salt thereof, wherein Y⁵ is O, NH, N(C₁₋₄ alkyl), or C(G^{A})(G^{B}), preferably, each of G^{A} and G^{B} is independently hydrogen, deuterium, halogen, or C₁₋₄ alkyl (e.g., methyl), or G^{A} and G^{B} together with the carbon atom they are both attached to are joined to form a 3-6 membered carbocyclic or heterocyclic ring, which is optionally substituted with one or more substituents each independently selected from halogen, OH, and C₁₋₄ alkyl (e.g., methyl), for example, Y⁵ is O or CH₂.

Embodiment 15. The compound of any of Embodiments 1-6 and 12-13, or a pharmaceutically acceptable salt thereof, wherein Y⁵, Y⁶, and Y⁷ together represent an optionally substituted 3-8 membered ring selected from 3-8 membered carbocyclic ring, 3-8 membered heterocyclic ring, phenyl ring, or heteroaryl ring, for example, Y⁵, Y⁶, and Y⁷ together represent

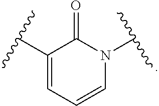 [SMILES: *c1cccn(*)c1=O]

Embodiment 16. The compound of any of Embodiments 1-6 and 12-14, or a pharmaceutically acceptable salt thereof, wherein Y⁶ is O, C(O) or C(G^{A})(G^{B}), preferably, each of G^{A} and G^{B} is independently hydrogen, deuterium, halogen, or C₁₋₄ alkyl (e.g., methyl), for example, Y⁶ is O, C(O) or CH₂.

Embodiment 17. The compound of any of Embodiments 1-6, 12-14, and 16, or a pharmaceutically acceptable salt thereof, wherein Y⁷ is O, C(O), NH, N(G^{C}), or C(G^{A})(G^{B}), preferably, each of G^{A} and G^{B} is independently hydrogen, deuterium, halogen, or C₁₋₄ alkyl (e.g., methyl), preferably, G^{C} is C₁₋₆ alkyl (e.g., methyl) or C₁₋₆ heteroalkyl, for example, Y⁷ is NH, O, CH₂, or N(CH₃).

Embodiment 18. The compound of any of Embodiments 1-6 and 12-14, or a pharmaceutically acceptable salt thereof, wherein Y⁶—Y⁷ is —C(O)NH—, —C(O)—N(G^{C})-, —NHCH₂—, or —OCH₂—, wherein G^{C} is C₁₋₆ alkyl (e.g., methyl) or C₁₋₆ heteroalkyl, for example, Y⁶-Y⁷ is —C(O)NH—, —C(O)—N(CH₃)—, —NHCH₂—, or —OCH₂—.

Embodiment 19. The compound of any of Embodiments 1-6 and 12, or a pharmaceutically acceptable salt thereof, wherein Y⁴, Y⁵, Y⁶, and Y⁷ together represent an optionally substituted 3-8 membered ring selected from 3-8 membered carbocyclic ring, 3-8 membered heterocyclic ring, phenyl ring, or heteroaryl ring, for example, Y⁴, Y⁵, Y⁶, and Y⁷ together represent

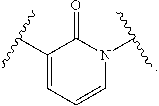 [SMILES: *c1cccn(*)c1=O]

Embodiment 20. The compound of any of Embodiments 1-5, or a pharmaceutically acceptable salt thereof, wherein Y², Y³, Y⁴, Y⁵, Y⁶, and Y⁷ together represent an optionally substituted 8-10 membered heterocyclic or heteroaryl ring.

Embodiment 21. The compound of any of Embodiments 1-6, or a pharmaceutically acceptable salt thereof, wherein Y³, Y⁴, Y⁵, Y⁶, and Y⁷ together represent an optionally substituted 8-10 membered heterocyclic or heteroaryl ring, preferably, a 6,5-fused or 6,6-fused heterocyclic or heteroaryl ring, having 1-5 ring heteroatoms each independently O, N, or S.

Embodiment 22. The compound of any of Embodiments 1-6, or a pharmaceutically acceptable salt thereof, wherein Y³, Y⁴, Y⁵, Y⁶, and Y⁷ together represent an optionally substituted 6,5-fused or 6,6-fused heterocyclic ring, in which a phenyl ring or 6-membered heteroaryl is fused with a 5- or 6-membered heterocyclic ring having 1 or 2 ring heteroatoms each independently O, N, or S, preferably, 1 ring nitrogen atom, for example, Y³, Y⁴, Y⁵, Y⁶, and Y⁷ together represent or

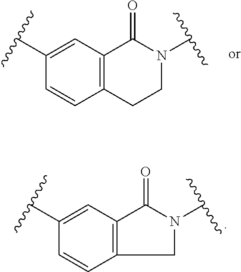 [SMILES: *c1ccc2c(c1)C(=O)N(*)CC2 ; *c1ccc2c(c1)C(=O)N(*)C2]

Embodiment 23. The compound of any of Embodiments 1-6, or a pharmaceutically acceptable salt thereof, wherein Y³, Y⁴, Y⁵, Y⁶, and Y⁷ together represent an optionally substituted 6,5-fused or 6,6-fused heteroaryl ring having 1-5 ring heteroatoms each independently O, N, or S, for example, an optionally substituted benzimidazole, indazole, benzothiophene, etc., when substituted, the 6,5-fused or 6,6-fused heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2} or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

Embodiment 24. The compound of any of Embodiments 1-23, or a pharmaceutically acceptable salt thereof, wherein Y⁸ is null.

Embodiment 25. The compound of any of Embodiments 1-23, or a pharmaceutically acceptable salt thereof, wherein Y⁸ is O, NH, C(O), C₁₋₂ alkylene, or C₁₋₆ heteroalkylene having 1-3 heteroatoms independently selected from O, N, and S, wherein the S is optionally oxidized, and the C₁₋₆ heteroalkylene is optionally substituted with 1 or 2 oxo groups.

Embodiment 26. The compound of any of Embodiments 1-25, or a pharmaceutically acceptable salt thereof, wherein Ring A1 is a 4-8 membered monocyclic carbocyclic or heterocyclic ring optionally substituted with one or more substituents each independently halogen, OH, CN, oxo, C₁₋₄ alkyl optionally substituted with F, or C₁₋₄ heteroalkyl optionally substituted with F.

Embodiment 27. The compound of any of Embodiments 1-25, or a pharmaceutically acceptable salt thereof, wherein Ring A1 is a C₄₋₇cycloalkylene optionally substituted with one or more substituents each independently halogen, OH, CN, oxo, C₁₋₄ alkyl optionally substituted with F, or C₁₋₄ heteroalkyl optionally substituted with F.

Embodiment 28. The compound of any of Embodiments 1-25, or a pharmaceutically acceptable salt thereof, wherein Ring A1 is cyclohexylene, preferably, a 1,4-trans-cyclohexylene,

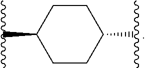 [SMILES: *[C@H]1CC[C@H](*)CC1]

Embodiment 29. The compound of any of Embodiments 1-28, or a pharmaceutically acceptable salt thereof, wherein T² is an optionally substituted 5,5-fused or 6,5-fused heteroaryl ring having 1-5 ring heteroatoms each independently O, N, or S, for example, an optionally substituted thiazolopyridine, imidazolopyridine, benzimidazole, pyrazolopyridine, oxazolopyridine, benzoxazole, indole, benzothiophene, benzothiazole, thienopyridine, thienopyrimidine, thienothiophene, etc., when substituted, the 5,5-fused or 6,5-fused heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

Embodiment 30. The compound of any of Embodiments 1-28, or a pharmaceutically acceptable salt thereof, wherein T² is an optionally substituted 6,5-fused heteroaryl ring having 1-5 ring heteroatoms each independently O, N, or S, wherein one of the fused rings is a furan, thiophene, pyrrole, pyrazole, imidazole, oxazole, thiazole, isoxazole, or isothiazole, which is fused with a benzene, pyridine, pyrimidine, pyridazine, or pyrazine, wherein when substituted, the 6,5-fused heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl, preferably, T² connects to Ring A1 through a ring atom on the 5-membered ring, for example, T² is an optionally substituted heteroaryl selected from:

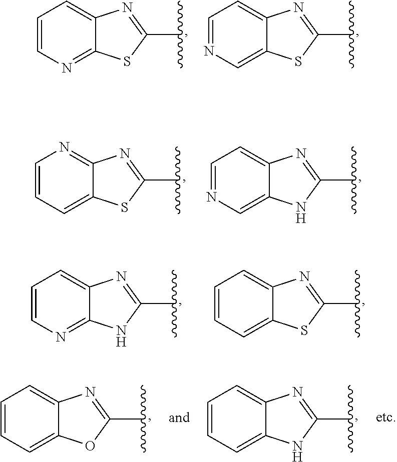

Embodiment 31. The compound of any of Embodiments 1-28, or a pharmaceutically acceptable salt thereof, wherein T² is an optionally substituted 5,6,5-fused, 6,5,5-fused, or 5,6,6-fused tricyclic heteroaryl ring having 1-5 ring heteroatoms each independently O, N, or S, wherein each 5-membered ring of the fused rings is independently a furan, thiophene, pyrrole, pyrazole, imidazole, oxazole, thiazole, isoxazole, or isothiazole, and each 6-membered ring of the fused rings is independently benzene, pyridine, pyrimidine, pyridazine, or pyrazine, wherein when substituted, the 5,6,5-fused, 6,5,5-fused, or 5,6,6-fused tricyclic heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl, for example, T² is an optionally substituted heteroaryl selected from:

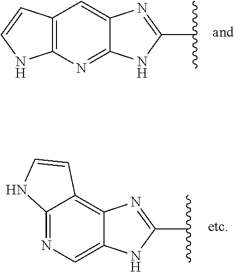 [SMILES: *c1nc2cc3cc[nH]c3nc2[nH]1 ; *c1nc2c(cnc3[nH]ccc32)[nH]1]

Embodiment 32. A compound of Formula B, or a pharmaceutically acceptable salt thereof,

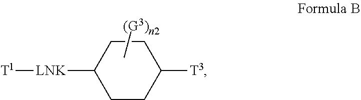

wherein:
T¹ is a glutarimide containing moiety;
T³ is an optionally substituted fused 8-14 membered bicyclic or tricyclic heteroaryl having 1-6 ring heteroatoms each independently selected from N, S, and O;
n2 is an integer of 0-2, and G³ at each occurrence is independently halogen, OH, CN, oxo, C₁₋₄ alkyl optionally substituted with F, or C₁₋₄ heteroalkyl optionally substituted with F; preferably, n2 is 0; and
LNK is a linker that connects T to the cyclohexyl ring, wherein the linker is a chain, ring, or a ring-chain structure, wherein the smallest number of chain or ring forming atoms of the linker is at least 4 (e.g., 4, 5, 6, 7, 8, or 9), wherein each of the chain or ring forming atoms is independently selected from C, N, O, and S, wherein the smallest number is the least number of atoms of the linker needed to reach from T¹ to the cyclohexyl ring, starting from the atom that is bonded to T¹ and ending with the atom that is bonded to the cyclohexyl ring.

Embodiment 33. The compound of Embodiment 32, or a pharmaceutically acceptable salt thereof, characterized as having a structure according to Formula B1:

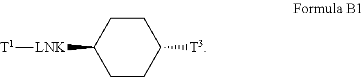

Embodiment 34. The compound of Embodiment 32 or 33, or a pharmaceutically acceptable salt thereof, wherein T³ is an optionally substituted 6,5-fused heteroaryl ring having 1-5 ring heteroatoms each independently O, N, or S, wherein one of the fused rings is a furan, thiophene, pyrrole, pyrazole, imidazole, oxazole, thiazole, isoxazole, or isothiazole, which is fused with a benzene, pyridine, pyrimidine, pyridazine, or pyrazine, wherein when substituted, the 6,5-fused heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl, preferably, T³ connects to the cyclohexyl ring through a ring atom on the 5-membered ring, for example, T³ is an optionally substituted heteroaryl selected from:

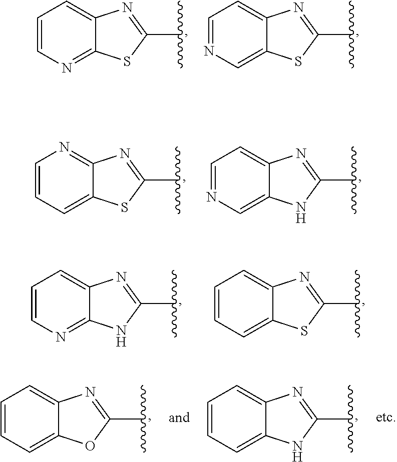

Embodiment 35. The compound of Embodiment 32 or 33, or a pharmaceutically acceptable salt thereof, wherein T³ is an optionally substituted 5,6,5-fused, 6,5,5-fused, or 5,6,6-fused tricyclic heteroaryl ring having 1-5 ring heteroatoms each independently O, N, or S, wherein each 5-membered ring of the fused rings is independently a furane, thiophene, pyrrole, pyrazole, imidazole, oxazole, thiazole, isoxazole, or isothiazole, and each 6-membered ring of the fused rings is independently benzene, pyridine, pyrimidine, pyridazine, or pyrazine, wherein when substituted, the 5,6,5-fused, 6,5,5-fused, or 5,6,6-fused tricyclic heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl, preferably, T³ connects to the cyclohexyl ring through a ring atom on a 5-membered ring, for example, T³ is an optionally substituted heteroaryl selected from:

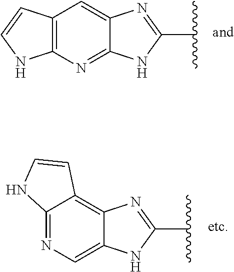 [SMILES: *c1nc2cc3cc[nH]c3nc2[nH]1 ; *c1nc2c(cnc3[nH]ccc32)[nH]1]

Embodiment 36. The compound of any of Embodiments 32-35, or a pharmaceutically acceptable salt thereof, wherein T¹ is a moiety having a structure according to

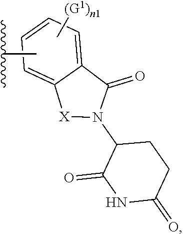

wherein:
X is CH₂ or C(═O),
n1 is an integer of 0-2, and
G¹ at each occurrence is independently halogen, CN, OH, NH₂, an optionally substituted C₁₋₆ alkyl, optionally substituted C₁₋₆ heteroalkyl, optionally substituted 3-8 membered carbocyclic or heterocyclic ring, optionally substituted phenyl, or optionally substituted heteroaryl.

Embodiment 37. The compound of Embodiment 36, or a pharmaceutically acceptable salt thereof, characterized as having a structure according to Formula B2:

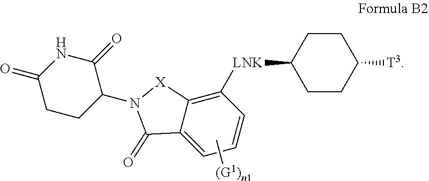

Embodiment 38. The compound of Embodiment 36 or 37, or a pharmaceutically acceptable salt thereof, wherein n1 is 0.

Embodiment 39. The compound of any of Embodiments 32-38, or a pharmaceutically acceptable salt thereof, wherein LNK is any linker defined herein, for example, a linker according to

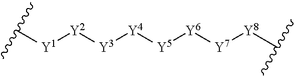

As defined in any of Embodiments 5-25, or any linker as defined herein for L in connection with Formula (I).

Embodiment 40. A compound of Formula C, or a pharmaceutically acceptable salt thereof,

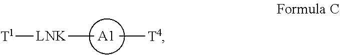

wherein:
T¹ is a glutarimide containing moiety;
LNK is a linker that connects T¹ to Ring A1, wherein the linker is a chain, ring, or a ring-chain structure, wherein the smallest number of chain or ring forming atoms of the linker is at least 4 (e.g., 4, 5, 6, 7, 8, or 9), wherein each of the chain or ring forming atoms is independently selected from C, N, O, and S, wherein the smallest number is the least number of atoms of the linker needed to reach from T¹ to Ring A1, starting from the atom that is bonded to T¹ and ending with the atom that is bonded to Ring A1;
Ring A1 is an optionally substituted 4-10 membered monocyclic or bicyclic carbocyclic or heterocyclic ring; and
T⁴ is an optionally substituted 5,5-fused or 6,5-fused heteroaryl ring having 1-5 ring heteroatoms each independently O, N, or S, or an optionally substituted 5,6,5-fused, 6,5,5-fused, or 5,6,6-fused tricyclic heteroaryl ring having 1-6 ring heteroatoms each independently O, N, or S, preferably, a ring atom of a 5-membered ring of T⁴ is bonded to Ring A1.

Embodiment 41. The compound of Embodiment 40, or a pharmaceutically acceptable salt thereof, wherein T⁴ is an optionally substituted 6,5-fused heteroaryl ring having 1-5 ring heteroatoms each independently O, N, or S, wherein one of the fused rings is a furan, thiophene, pyrrole, pyrazole, imidazole, oxazole, thiazole, isoxazole, or isothiazole, which is fused with a benzene, pyridine, pyrimidine, pyridazine, or pyrazine, wherein when substituted, the 6,5-fused heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl, for example, T⁴ is an optionally substituted heteroaryl selected from:

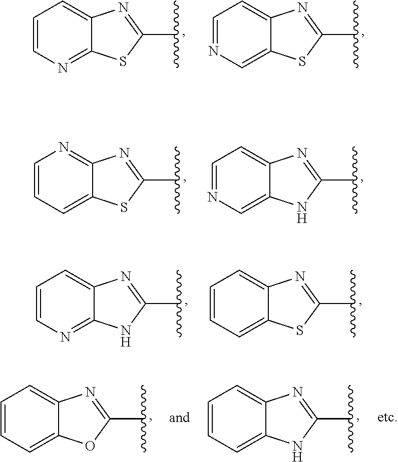

Embodiment 42. The compound of Embodiment 40, or a pharmaceutically acceptable salt thereof, wherein T⁴ is an optionally substituted 5,6,5-fused, 6,5,5-fused, or 5,6,6-fused tricyclic heteroaryl ring having 1-5 ring heteroatoms each independently O, N, or S, wherein each 5-membered ring of the fused rings is independently a furan, thiophene, pyrrole, pyrazole, imidazole, oxazole, thiazole, isoxazole, or isothiazole, and each 6-membered ring of the fused rings is independently benzene, pyridine, pyrimidine, pyridazine, or pyrazine, wherein when substituted, the 5,6,5-fused, 6,5,5-fused, or 5,6,6-fused tricyclic heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁ 4 heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl, for example, T⁴ is an optionally substituted heteroaryl selected from:

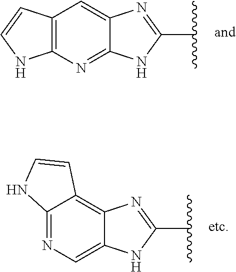 [SMILES: *c1nc2cc3cc[nH]c3nc2[nH]1 ; *c1nc2c(cnc3[nH]ccc32)[nH]1]

Embodiment 43. The compound of any of Embodiments 40-42, or a pharmaceutically acceptable salt thereof, wherein T¹ is a moiety having a structure according to

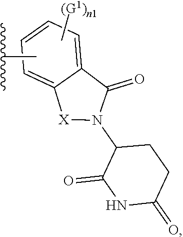

wherein:
X is CH₂ or C(═O),
n1 is an integer of 0-2, and
G¹ at each occurrence is independently halogen, CN, OH, NH₂, an optionally substituted
C₁₋₆ alkyl, optionally substituted C₁₋₆ heteroalkyl, optionally substituted 3-8 membered carbocyclic or heterocyclic ring, optionally substituted phenyl, or optionally substituted heteroaryl.

Embodiment 44. The compound of Embodiment 43, or a pharmaceutically acceptable salt thereof, characterized as having a structure according to Formula C₁:

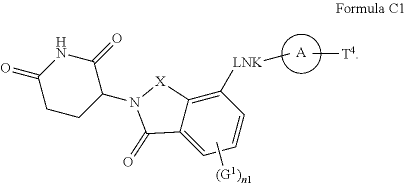

Embodiment 45. The compound of Embodiment 43 or 44, or a pharmaceutically acceptable salt thereof, wherein n1 is 0.

Embodiment 46. The compound of any of Embodiments 40-45, or a pharmaceutically acceptable salt thereof, wherein LNK is any linker defined herein, for example, a linker according to

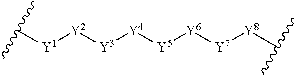

As defined in any of Embodiments 5-25, or any linker as defined herein for L in connection with Formula (I).

Embodiment 47. The compound of any of Embodiments 40-46, or a pharmaceutically acceptable salt thereof, wherein Ring A1 is an optionally substituted 4-8 membered monocyclic carbocyclic or heterocyclic ring.

Embodiment 48. The compound of any of Embodiments 40-46, or a pharmaceutically acceptable salt thereof, wherein Ring A1 is an optionally substituted C₄₋₇cycloalkylene.

Embodiment 49. The compound of any of Embodiments 40-46, or a pharmaceutically acceptable salt thereof, wherein Ring A1 is cyclohexylene, preferably, a 1,4-trans-cyclohexylene,

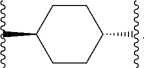 [SMILES: *[C@H]1CC[C@H](*)CC1]

Embodiment 50. A compound selected from any of Examples 1-150, or a pharmaceutically acceptable salt thereof. Structures of Examples 1-150 can be found in the Synthesis Examples section.

Embodiment 51. A pharmaceutical composition comprising the compound of any of Embodiments 1-50 or a pharmaceutically acceptable salt thereof and a pharmaceutically acceptable excipient.

Embodiment 52. A method of inducing degradation of a protein in a cell, the method comprising contacting the cell with the compound of any of Embodiments 1-50 or a pharmaceutically acceptable salt thereof.

Embodiment 53. The method of Embodiment 52, wherein the protein is GSPT1.

Embodiment 54. The method of Embodiment 52 or 53, wherein the cell is a cancer cell.

Embodiment 55. A method of treating cancer in a subject in need thereof, the method comprising administering to the subject a therapeutically effective amount of the compound of any of Embodiments 1-50 or a pharmaceutically acceptable salt thereof or the pharmaceutical composition of Embodiment 51.

Embodiment 56. The method of Embodiment 55, wherein the cancer is associated with GSPT1 activity.

In some embodiments of the present invention, the compound of the present invention is selected from the following structures:

| ID | Structure |
|---|---|
| A1 | 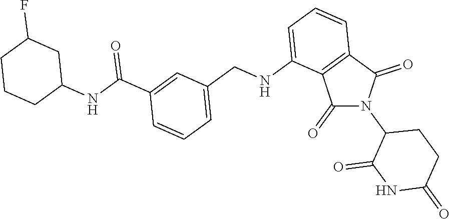 [SMILES: O=C1CCC(N2C(=O)c3cccc(NCc4cccc(C(=O)NC5CCCC(F)C5)c4)c3C2=O)C(=O)N1] |
| A2 | 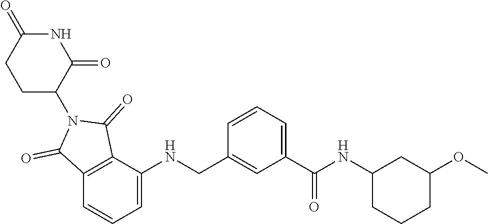 [SMILES: COC1CCCC(NC(=O)c2cccc(CNc3cccc4c3C(=O)N(C3CCC(=O)NC3=O)C4=O)c2)C1] |
| A3 | 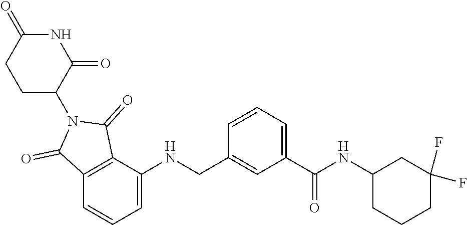 [SMILES: O=C1CCC(N2C(=O)c3cccc(NCc4cccc(C(=O)NC5CCCC(F)(F)C5)c4)c3C2=O)C(=O)N1] |
| A4 | 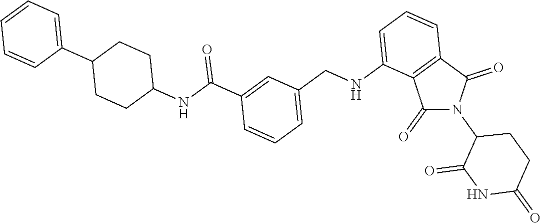 [SMILES: O=C1CCC(N2C(=O)c3cccc(NCc4cccc(C(=O)NC5CCC(c6ccccc6)CC5)c4)c3C2=O)C(=O)N1] |
| A5 | 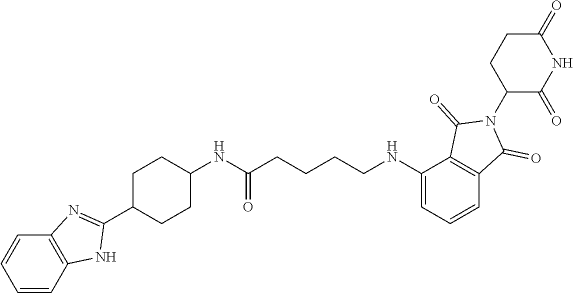 [SMILES: O=C1CCC(N2C(=O)c3cccc(NCCCCC(=O)NC4CCC(c5nc6ccccc6[nH]5)CC4)c3C2=O)C(=O)N1] |
| A6 | 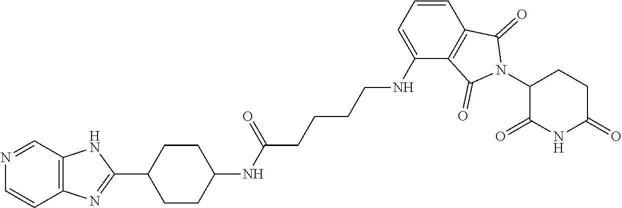 [SMILES: O=C1CCC(N2C(=O)c3cccc(NCCCCC(=O)NC4CCC(c5nc6ccncc6[nH]5)CC4)c3C2=O)C(=O)N1] |
| A7 | 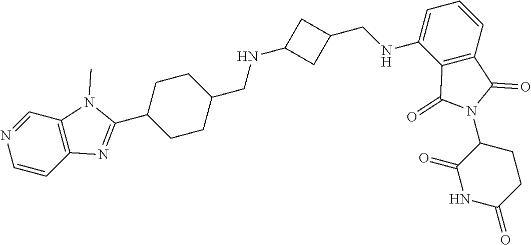 [SMILES: Cn1c(C2CCC(CNC3CC(CNc4cccc5c4C(=O)N(C4CCC(=O)NC4=O)C5=O)C3)CC2)nc2ccncc21] |
| A8 | 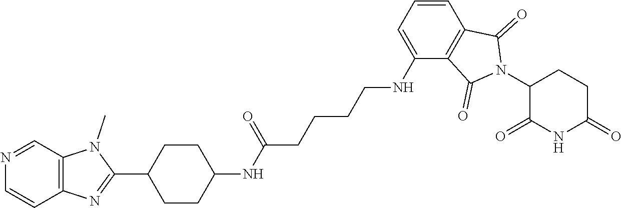 [SMILES: Cn1c(C2CCC(NC(=O)CCCCNc3cccc4c3C(=O)N(C3CCC(=O)NC3=O)C4=O)CC2)nc2ccncc21] |
| A9 | 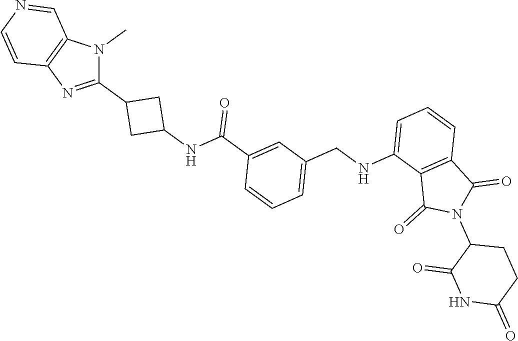 [SMILES: Cn1c(C2CC(NC(=O)c3cccc(CNc4cccc5c4C(=O)N(C4CCC(=O)NC4=O)C5=O)c3)C2)nc2ccncc21] |
| A10 | 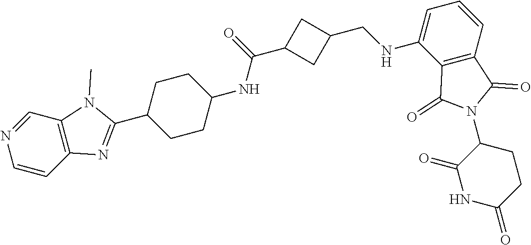 [SMILES: Cn1c(C2CCC(NC(=O)C3CC(CNc4cccc5c4C(=O)N(C4CCC(=O)NC4=O)C5=O)C3)CC2)nc2ccncc21] |
| A11 | 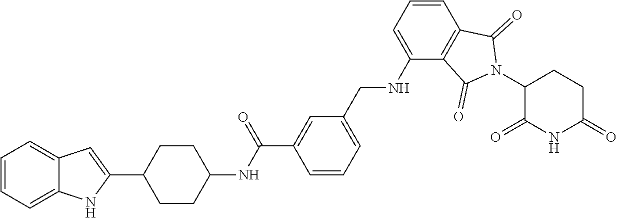 [SMILES: O=C1CCC(N2C(=O)c3cccc(NCc4cccc(C(=O)NC5CCC(c6cc7ccccc7[nH]6)CC5)c4)c3C2=O)C(=O)N1] |
| A12 | 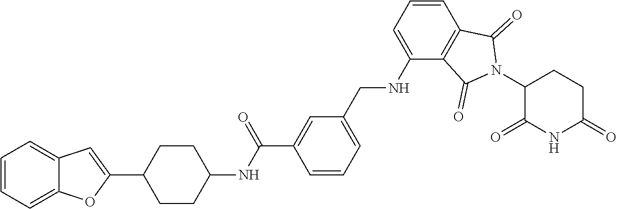 [SMILES: O=C1CCC(N2C(=O)c3cccc(NCc4cccc(C(=O)NC5CCC(c6cc7ccccc7o6)CC5)c4)c3C2=O)C(=O)N1] |
| A13 | 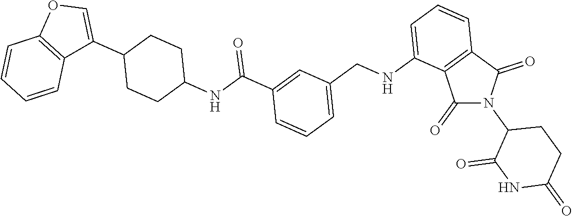 [SMILES: O=C1CCC(N2C(=O)c3cccc(NCc4cccc(C(=O)NC5CCC(c6coc7ccccc67)CC5)c4)c3C2=O)C(=O)N1] |
| A14 | 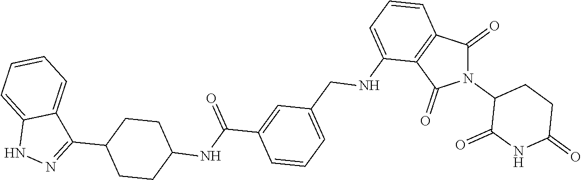 [SMILES: O=C1CCC(N2C(=O)c3cccc(NCc4cccc(C(=O)NC5CCC(c6n[nH]c7ccccc67)CC5)c4)c3C2=O)C(=O)N1] |
| A15 | 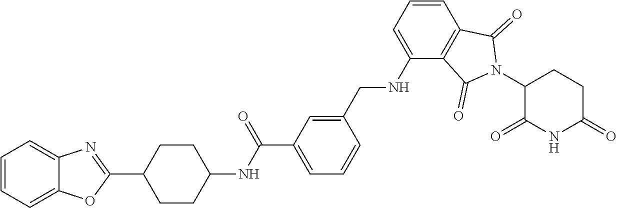 [SMILES: O=C1CCC(N2C(=O)c3cccc(NCc4cccc(C(=O)NC5CCC(c6nc7ccccc7o6)CC5)c4)c3C2=O)C(=O)N1] |
| A16 | 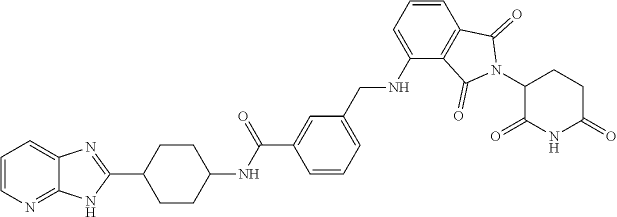 [SMILES: O=C1CCC(N2C(=O)c3cccc(NCc4cccc(C(=O)NC5CCC(c6nc7cccnc7[nH]6)CC5)c4)c3C2=O)C(=O)N1] |
| A17 | 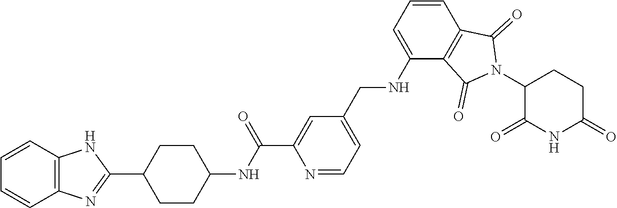 [SMILES: O=C1CCC(N2C(=O)c3cccc(NCc4ccnc(C(=O)NC5CCC(c6nc7ccccc7[nH]6)CC5)c4)c3C2=O)C(=O)N1] |
| A18 | 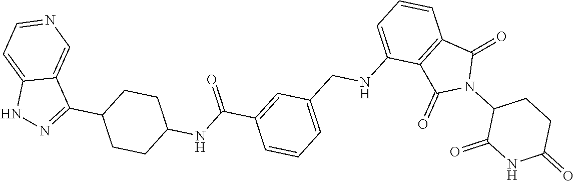 [SMILES: O=C1CCC(N2C(=O)c3cccc(NCc4cccc(C(=O)NC5CCC(c6n[nH]c7ccncc67)CC5)c4)c3C2=O)C(=O)N1] |
| A19 | 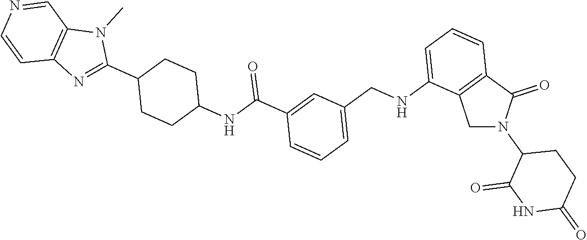 [SMILES: Cn1c(C2CCC(NC(=O)c3cccc(CNc4cccc5c4CN(C4CCC(=O)NC4=O)C5=O)c3)CC2)nc2ccncc21] |
| A20 | 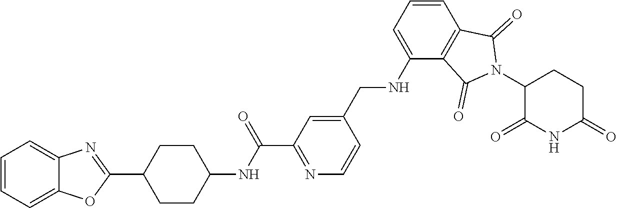 [SMILES: O=C1CCC(N2C(=O)c3cccc(NCc4ccnc(C(=O)NC5CCC(c6nc7ccccc7o6)CC5)c4)c3C2=O)C(=O)N1] |
| A21 | 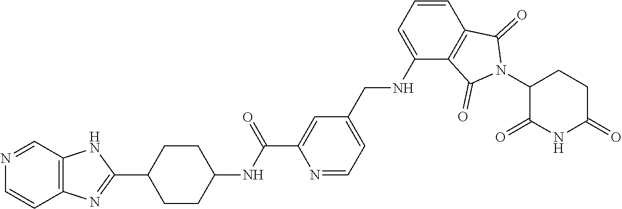 [SMILES: O=C1CCC(N2C(=O)c3cccc(NCc4ccnc(C(=O)NC5CCC(c6nc7ccncc7[nH]6)CC5)c4)c3C2=O)C(=O)N1] |
| A22 | 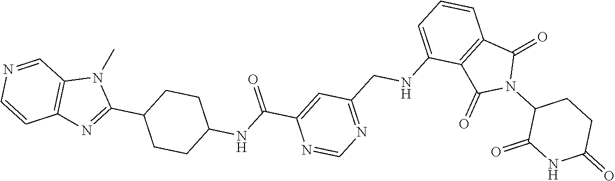 [SMILES: Cn1c(C2CCC(NC(=O)c3cc(CNc4cccc5c4C(=O)N(C4CCC(=O)NC4=O)C5=O)ncn3)CC2)nc2ccncc21] |
| A23 | 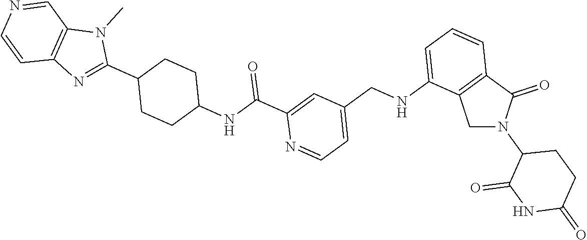 [SMILES: Cn1c(C2CCC(NC(=O)c3cc(CNc4cccc5c4CN(C4CCC(=O)NC4=O)C5=O)ccn3)CC2)nc2ccncc21] |
| A24 | 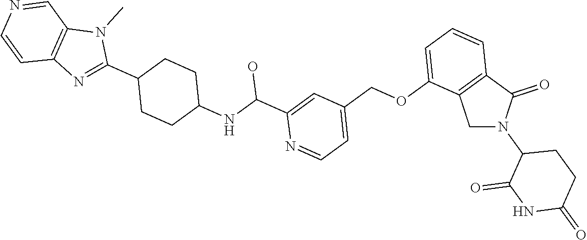 |
| A25 | 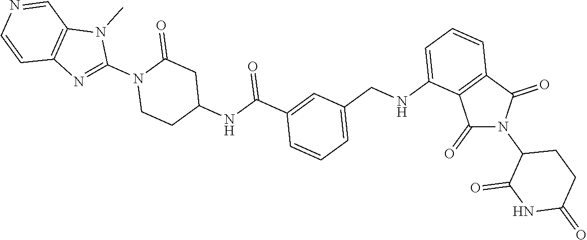 [SMILES: Cn1c(N2CCC(NC(=O)c3cccc(CNc4cccc5c4C(=O)N(C4CCC(=O)NC4=O)C5=O)c3)CC2=O)nc2ccncc21] |
| A26 | 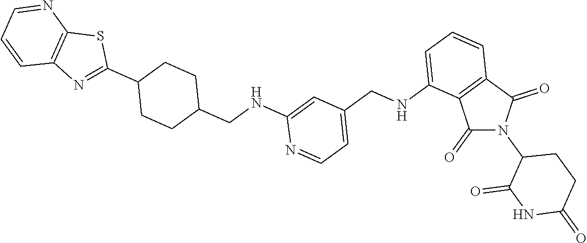 [SMILES: O=C1CCC(N2C(=O)c3cccc(NCc4ccnc(NCC5CCC(c6nc7cccnc7s6)CC5)c4)c3C2=O)C(=O)N1] |
| A27 | 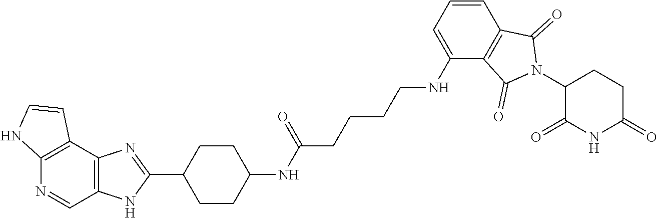 [SMILES: O=C1CCC(N2C(=O)c3cccc(NCCCCC(=O)NC4CCC(c5nc6c(cnc7[nH]ccc76)[nH]5)CC4)c3C2=O)C(=O)N1] |
| A28 | 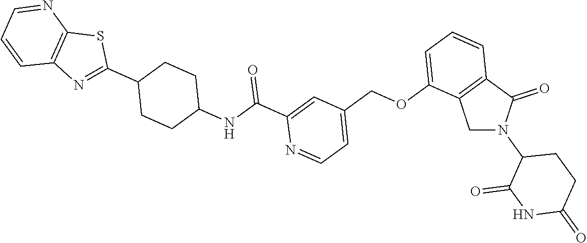 [SMILES: O=C1CCC(N2Cc3c(OCc4ccnc(C(=O)NC5CCC(c6nc7cccnc7s6)CC5)c4)cccc3C2=O)C(=O)N1] |
| A29 | 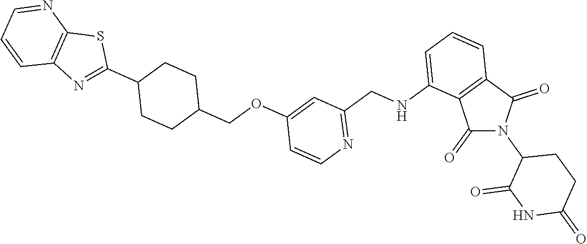 [SMILES: O=C1CCC(N2C(=O)c3cccc(NCc4cc(OCC5CCC(c6nc7cccnc7s6)CC5)ccn4)c3C2=O)C(=O)N1] |
| A30 | 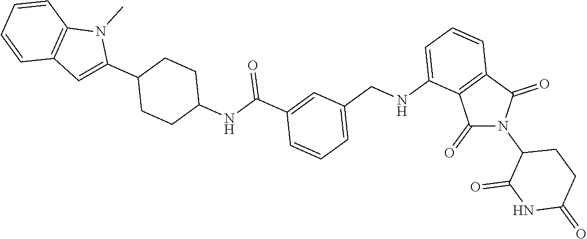 [SMILES: Cn1c(C2CCC(NC(=O)c3cccc(CNc4cccc5c4C(=O)N(C4CCC(=O)NC4=O)C5=O)c3)CC2)cc2ccccc21] |
| A31 | 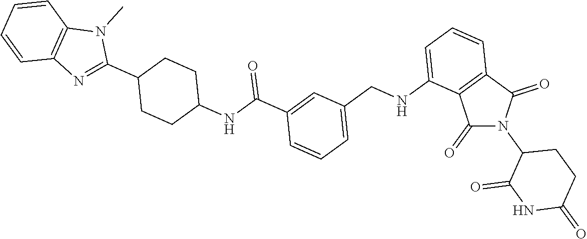 [SMILES: Cn1c(C2CCC(NC(=O)c3cccc(CNc4cccc5c4C(=O)N(C4CCC(=O)NC4=O)C5=O)c3)CC2)nc2ccccc21] |
| A32 | 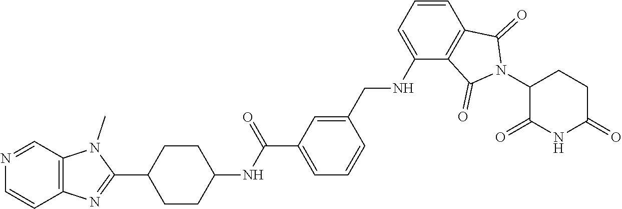 [SMILES: Cn1c(C2CCC(NC(=O)c3cccc(CNc4cccc5c4C(=O)N(C4CCC(=O)NC4=O)C5=O)c3)CC2)nc2ccncc21] |
| A33 | 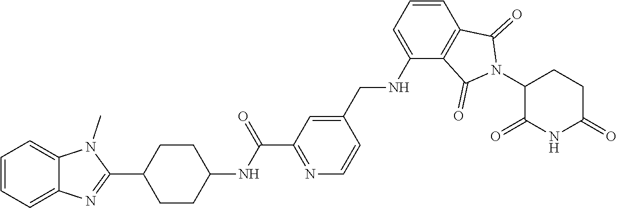 [SMILES: Cn1c(C2CCC(NC(=O)c3cc(CNc4cccc5c4C(=O)N(C4CCC(=O)NC4=O)C5=O)ccn3)CC2)nc2ccccc21] |
| A34 | 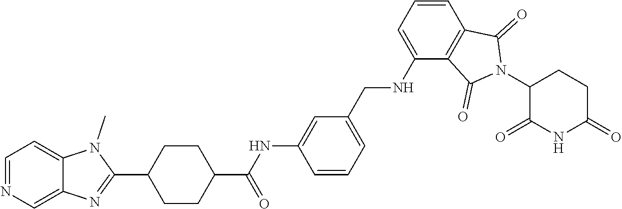 [SMILES: Cn1c(C2CCC(C(=O)Nc3cccc(CNc4cccc5c4C(=O)N(C4CCC(=O)NC4=O)C5=O)c3)CC2)nc2cnccc21] |
| A35 | 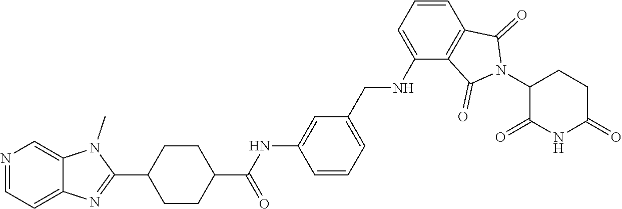 [SMILES: Cn1c(C2CCC(C(=O)Nc3cccc(CNc4cccc5c4C(=O)N(C4CCC(=O)NC4=O)C5=O)c3)CC2)nc2ccncc21] |
| A36 | 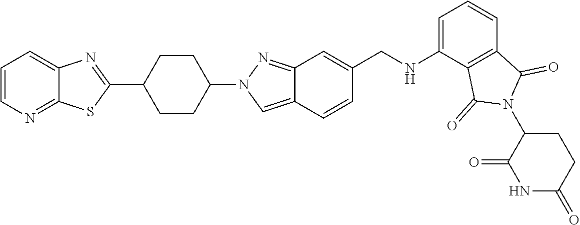 [SMILES: O=C1CCC(N2C(=O)c3cccc(NCc4ccc5cn(C6CCC(c7nc8cccnc8s7)CC6)nc5c4)c3C2=O)C(=O)N1] |
| A37 | 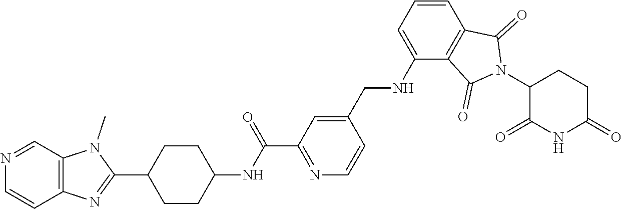 [SMILES: Cn1c(C2CCC(NC(=O)c3cc(CNc4cccc5c4C(=O)N(C4CCC(=O)NC4=O)C5=O)ccn3)CC2)nc2ccncc21] |
| A38 | 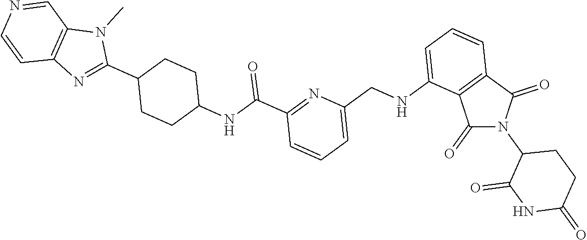 [SMILES: Cn1c(C2CCC(NC(=O)c3cccc(CNc4cccc5c4C(=O)N(C4CCC(=O)NC4=O)C5=O)n3)CC2)nc2ccncc21] |
| A39 | 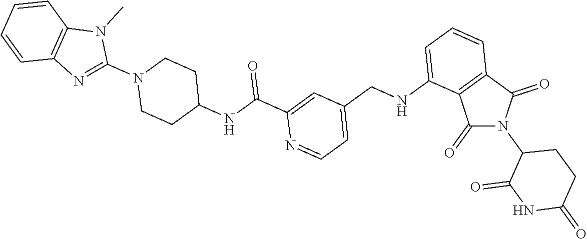 [SMILES: Cn1c(N2CCC(NC(=O)c3cc(CNc4cccc5c4C(=O)N(C4CCC(=O)NC4=O)C5=O)ccn3)CC2)nc2ccccc21] |
| A40 | 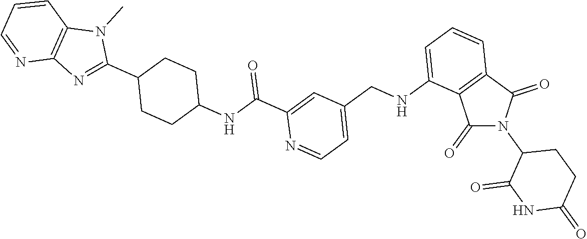 [SMILES: Cn1c(C2CCC(NC(=O)c3cc(CNc4cccc5c4C(=O)N(C4CCC(=O)NC4=O)C5=O)ccn3)CC2)nc2ncccc21] |
| A41 | 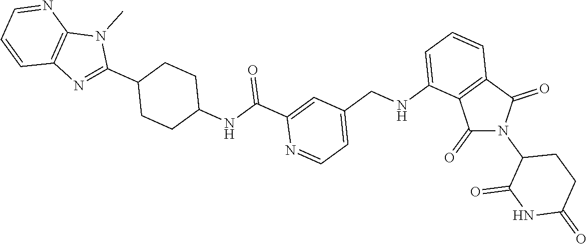 [SMILES: Cn1c(C2CCC(NC(=O)c3cc(CNc4cccc5c4C(=O)N(C4CCC(=O)NC4=O)C5=O)ccn3)CC2)nc2cccnc21] |
| A42 | 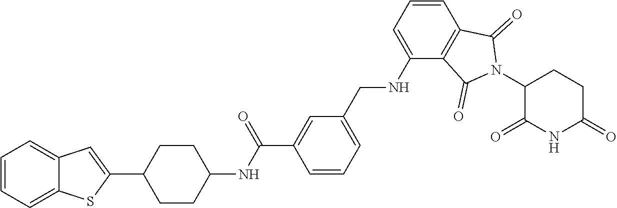 [SMILES: O=C1CCC(N2C(=O)c3cccc(NCc4cccc(C(=O)NC5CCC(c6cc7ccccc7s6)CC5)c4)c3C2=O)C(=O)N1] |
| A43 | 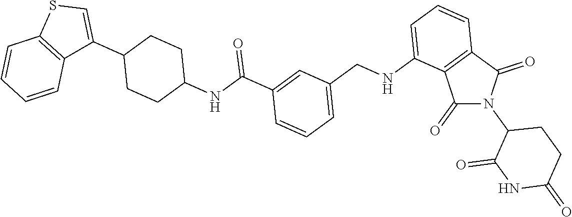 [SMILES: O=C1CCC(N2C(=O)c3cccc(NCc4cccc(C(=O)NC5CCC(c6csc7ccccc67)CC5)c4)c3C2=O)C(=O)N1] |
| A44 | 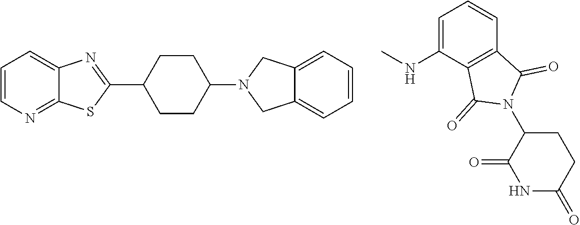 |
| A45 | 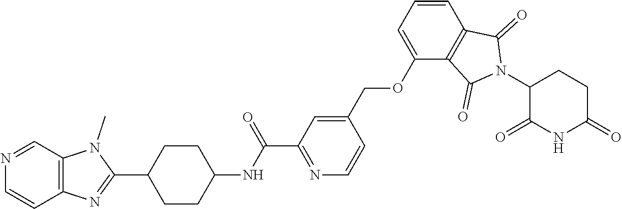 [SMILES: Cn1c(C2CCC(NC(=O)c3cc(COc4cccc5c4C(=O)N(C4CCC(=O)NC4=O)C5=O)ccn3)CC2)nc2ccncc21] |
| A46 | 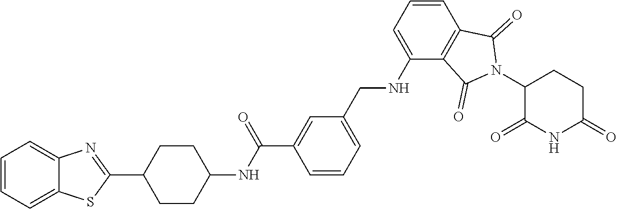 [SMILES: O=C1CCC(N2C(=O)c3cccc(NCc4cccc(C(=O)NC5CCC(c6nc7ccccc7s6)CC5)c4)c3C2=O)C(=O)N1] |
| A47 | 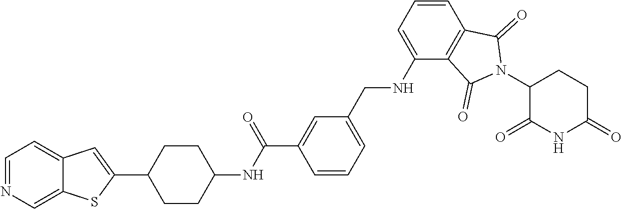 [SMILES: O=C1CCC(N2C(=O)c3cccc(NCc4cccc(C(=O)NC5CCC(c6cc7ccncc7s6)CC5)c4)c3C2=O)C(=O)N1] |
| A48 | 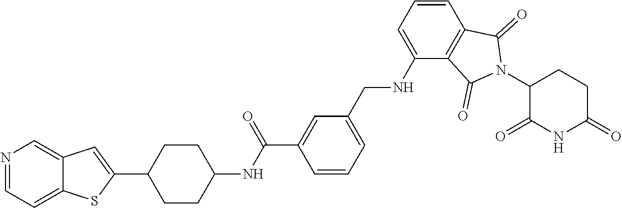 [SMILES: O=C1CCC(N2C(=O)c3cccc(NCc4cccc(C(=O)NC5CCC(c6cc7cnccc7s6)CC5)c4)c3C2=O)C(=O)N1] |
| A49 | 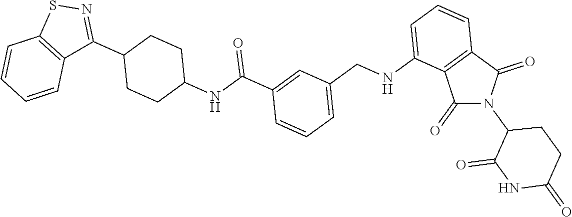 [SMILES: O=C1CCC(N2C(=O)c3cccc(NCc4cccc(C(=O)NC5CCC(c6nsc7ccccc67)CC5)c4)c3C2=O)C(=O)N1] |
| A50 | 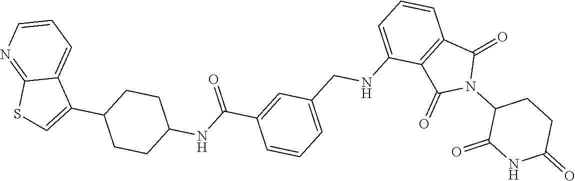 [SMILES: O=C1CCC(N2C(=O)c3cccc(NCc4cccc(C(=O)NC5CCC(c6csc7ncccc67)CC5)c4)c3C2=O)C(=O)N1] |
| A51 | 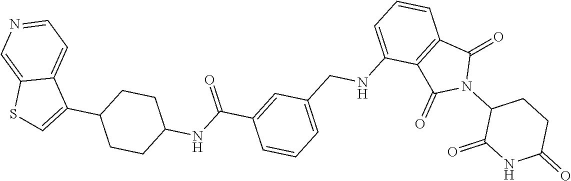 [SMILES: O=C1CCC(N2C(=O)c3cccc(NCc4cccc(C(=O)NC5CCC(c6csc7cnccc67)CC5)c4)c3C2=O)C(=O)N1] |
| A52 | 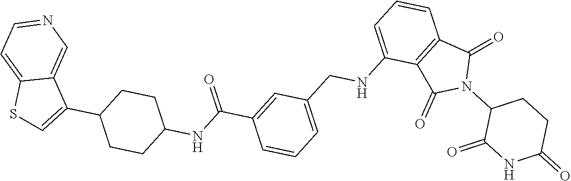 [SMILES: O=C1CCC(N2C(=O)c3cccc(NCc4cccc(C(=O)NC5CCC(c6csc7ccncc67)CC5)c4)c3C2=O)C(=O)N1] |
| A53 | 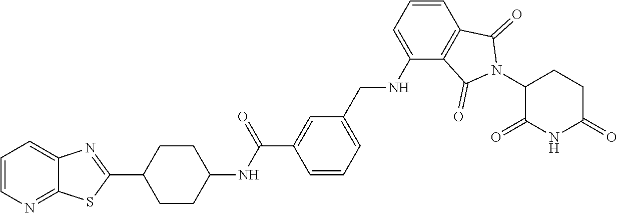 [SMILES: O=C1CCC(N2C(=O)c3cccc(NCc4cccc(C(=O)NC5CCC(c6nc7cccnc7s6)CC5)c4)c3C2=O)C(=O)N1] |
| A54 | 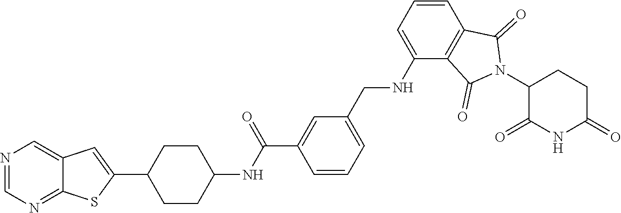 [SMILES: O=C1CCC(N2C(=O)c3cccc(NCc4cccc(C(=O)NC5CCC(c6cc7cncnc7s6)CC5)c4)c3C2=O)C(=O)N1] |
| A55 | 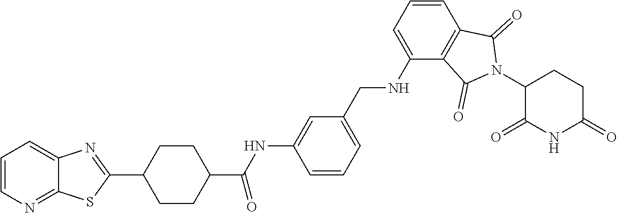 [SMILES: O=C1CCC(N2C(=O)c3cccc(NCc4cccc(NC(=O)C5CCC(c6nc7cccnc7s6)CC5)c4)c3C2=O)C(=O)N1] |
| A56 | 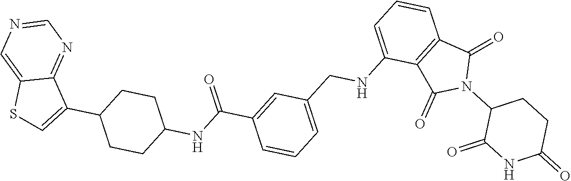 [SMILES: O=C1CCC(N2C(=O)c3cccc(NCc4cccc(C(=O)NC5CCC(c6csc7cncnc67)CC5)c4)c3C2=O)C(=O)N1] |
| A57 | 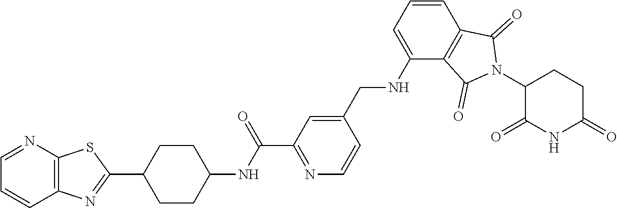 [SMILES: O=C1CCC(N2C(=O)c3cccc(NCc4ccnc(C(=O)NC5CCC(c6nc7cccnc7s6)CC5)c4)c3C2=O)C(=O)N1] |
| A58 | 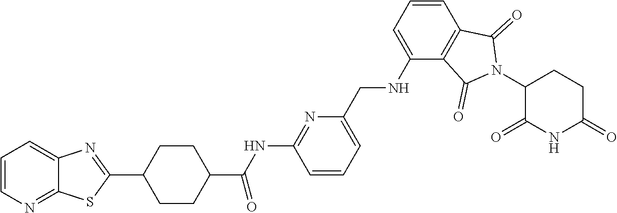 [SMILES: O=C1CCC(N2C(=O)c3cccc(NCc4cccc(NC(=O)C5CCC(c6nc7cccnc7s6)CC5)n4)c3C2=O)C(=O)N1] |
| A59 | 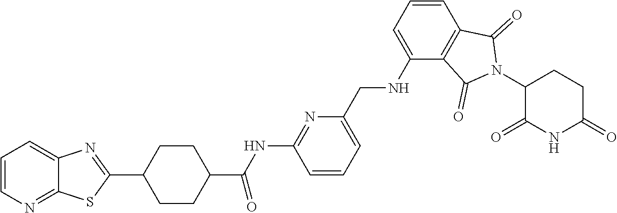 [SMILES: O=C1CCC(N2C(=O)c3cccc(NCc4cccc(NC(=O)C5CCC(c6nc7cccnc7s6)CC5)n4)c3C2=O)C(=O)N1] |
| A60 | 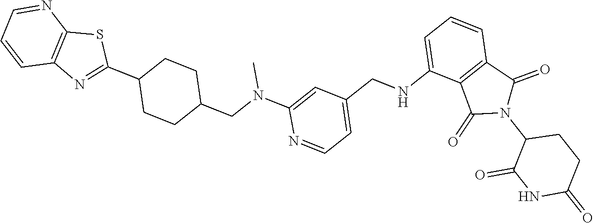 [SMILES: CN(CC1CCC(c2nc3cccnc3s2)CC1)c1cc(CNc2cccc3c2C(=O)N(C2CCC(=O)NC2=O)C3=O)ccn1] |
| A61 | 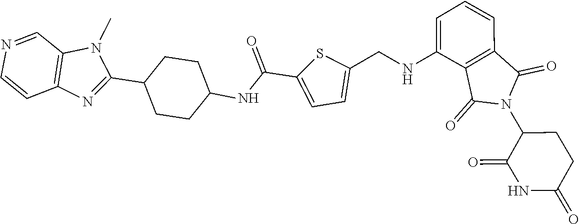 [SMILES: Cn1c(C2CCC(NC(=O)c3ccc(CNc4cccc5c4C(=O)N(C4CCC(=O)NC4=O)C5=O)s3)CC2)nc2ccncc21] |
| A62 | 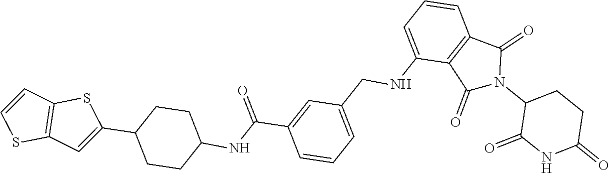 [SMILES: O=C1CCC(N2C(=O)c3cccc(NCc4cccc(C(=O)NC5CCC(c6cc7sccc7s6)CC5)c4)c3C2=O)C(=O)N1] |
| A63 | 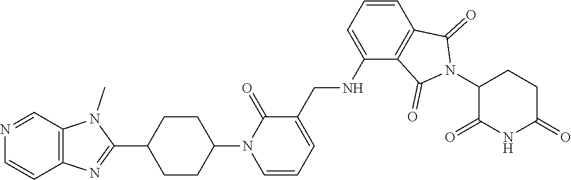 [SMILES: Cn1c(C2CCC(n3cccc(CNc4cccc5c4C(=O)N(C4CCC(=O)NC4=O)C5=O)c3=O)CC2)nc2ccncc21] |
| A64 | 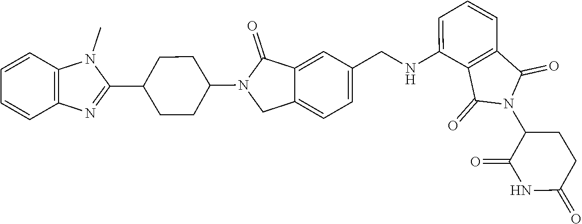 [SMILES: Cn1c(C2CCC(N3Cc4ccc(CNc5cccc6c5C(=O)N(C5CCC(=O)NC5=O)C6=O)cc4C3=O)CC2)nc2ccccc21] |
| A65 | 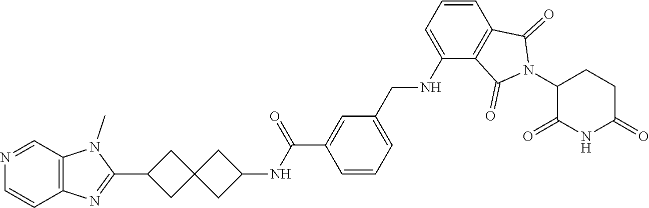 [SMILES: Cn1c(C2CC3(CC(NC(=O)c4cccc(CNc5cccc6c5C(=O)N(C5CCC(=O)NC5=O)C6=O)c4)C3)C2)nc2ccncc21] |
| A66 | 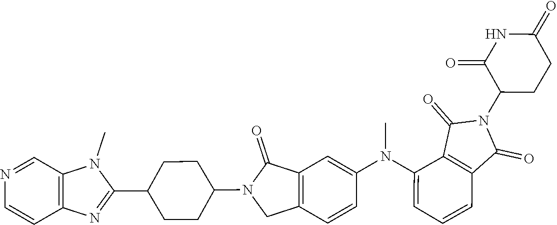 [SMILES: CN(c1ccc2c(c1)C(=O)N(C1CCC(c3nc4ccncc4n3C)CC1)C2)c1cccc2c1C(=O)N(C1CCC(=O)NC1=O)C2=O] |
| A67 | 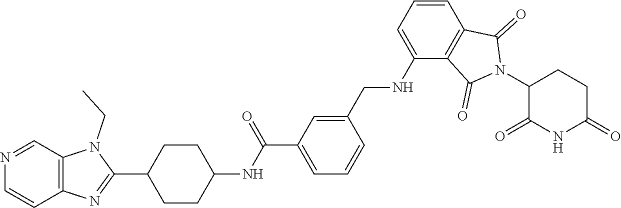 [SMILES: CCn1c(C2CCC(NC(=O)c3cccc(CNc4cccc5c4C(=O)N(C4CCC(=O)NC4=O)C5=O)c3)CC2)nc2ccncc21] |
| A68 | 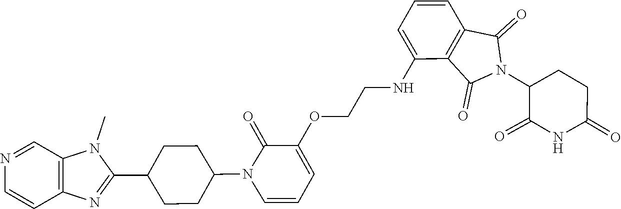 [SMILES: Cn1c(C2CCC(n3cccc(OCCNc4cccc5c4C(=O)N(C4CCC(=O)NC4=O)C5=O)c3=O)CC2)nc2ccncc21] |
| A69 | 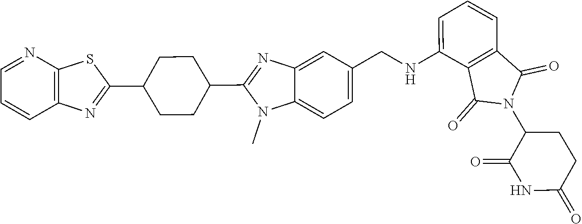 [SMILES: Cn1c(C2CCC(c3nc4cccnc4s3)CC2)nc2cc(CNc3cccc4c3C(=O)N(C3CCC(=O)NC3=O)C4=O)ccc21] |
| A70 | 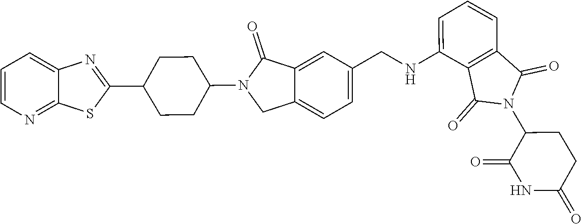 [SMILES: O=C1CCC(N2C(=O)c3cccc(NCc4ccc5c(c4)C(=O)N(C4CCC(c6nc7cccnc7s6)CC4)C5)c3C2=O)C(=O)N1] |
| A71 | 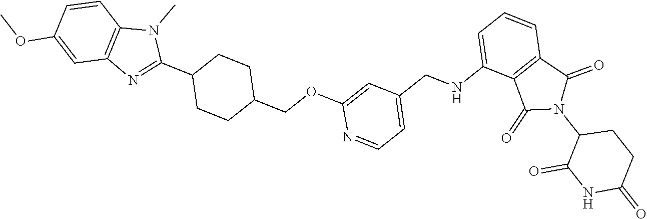 [SMILES: COc1ccc2c(c1)nc(C1CCC(COc3cc(CNc4cccc5c4C(=O)N(C4CCC(=O)NC4=O)C5=O)ccn3)CC1)n2C] |
| A72 | 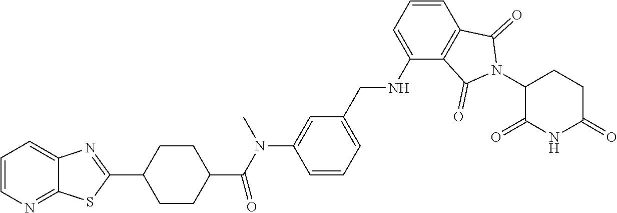 [SMILES: CN(C(=O)C1CCC(c2nc3cccnc3s2)CC1)c1cccc(CNc2cccc3c2C(=O)N(C2CCC(=O)NC2=O)C3=O)c1] |
| A73 | 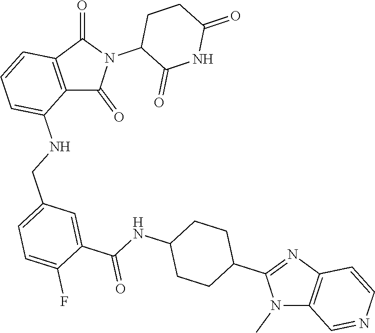 [SMILES: Cn1c(C2CCC(NC(=O)c3cc(CNc4cccc5c4C(=O)N(C4CCC(=O)NC4=O)C5=O)ccc3F)CC2)nc2ccncc21] |
| A74 | 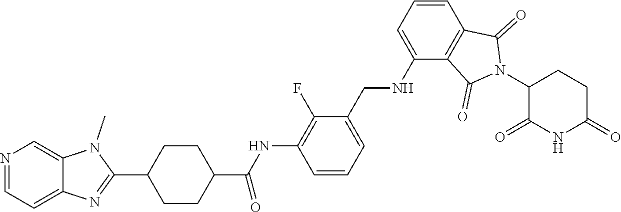 [SMILES: Cn1c(C2CCC(C(=O)Nc3cccc(CNc4cccc5c4C(=O)N(C4CCC(=O)NC4=O)C5=O)c3F)CC2)nc2ccncc21] |
| A75 | 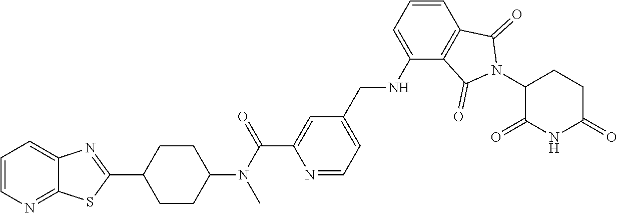 [SMILES: CN(C(=O)c1cc(CNc2cccc3c2C(=O)N(C2CCC(=O)NC2=O)C3=O)ccn1)C1CCC(c2nc3cccnc3s2)CC1] |
| A76 | 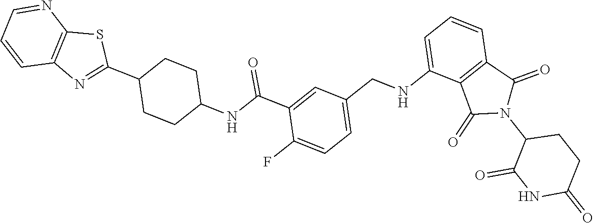 [SMILES: O=C1CCC(N2C(=O)c3cccc(NCc4ccc(F)c(C(=O)NC5CCC(c6nc7cccnc7s6)CC5)c4)c3C2=O)C(=O)N1] |
| A77 | 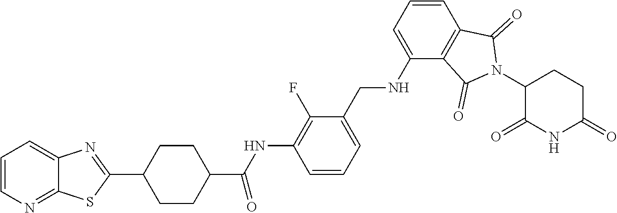 [SMILES: O=C1CCC(N2C(=O)c3cccc(NCc4cccc(NC(=O)C5CCC(c6nc7cccnc7s6)CC5)c4F)c3C2=O)C(=O)N1] |
| A78 | 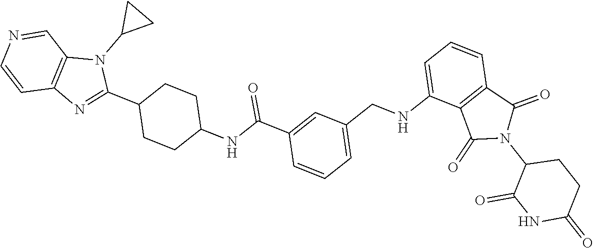 [SMILES: O=C1CCC(N2C(=O)c3cccc(NCc4cccc(C(=O)NC5CCC(c6nc7ccncc7n6C6CC6)CC5)c4)c3C2=O)C(=O)N1] |
| A79 | 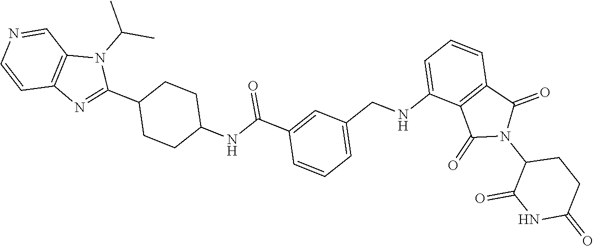 [SMILES: CC(C)n1c(C2CCC(NC(=O)c3cccc(CNc4cccc5c4C(=O)N(C4CCC(=O)NC4=O)C5=O)c3)CC2)nc2ccncc21] |
| A80 | 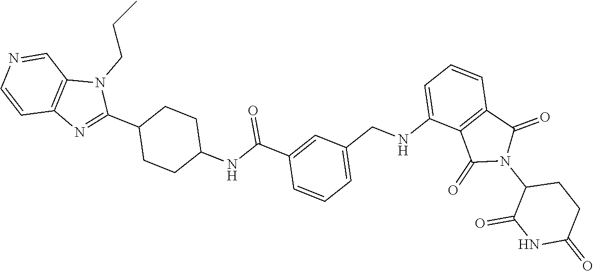 [SMILES: CCCn1c(C2CCC(NC(=O)c3cccc(CNc4cccc5c4C(=O)N(C4CCC(=O)NC4=O)C5=O)c3)CC2)nc2ccncc21] |
| A81 | 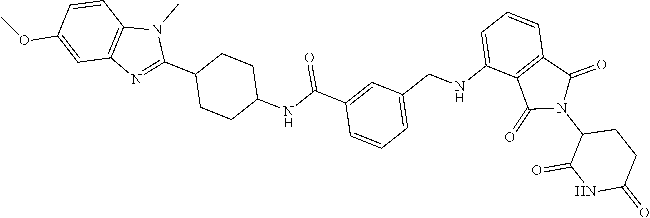 [SMILES: COc1ccc2c(c1)nc(C1CCC(NC(=O)c3cccc(CNc4cccc5c4C(=O)N(C4CCC(=O)NC4=O)C5=O)c3)CC1)n2C] |
| A82 | 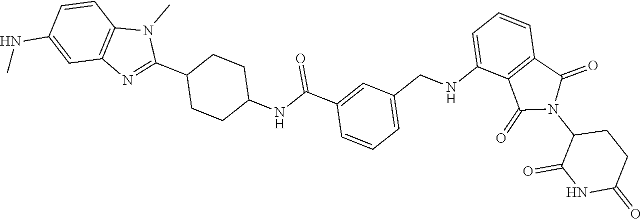 [SMILES: CNc1ccc2c(c1)nc(C1CCC(NC(=O)c3cccc(CNc4cccc5c4C(=O)N(C4CCC(=O)NC4=O)C5=O)c3)CC1)n2C] |
| A83 | 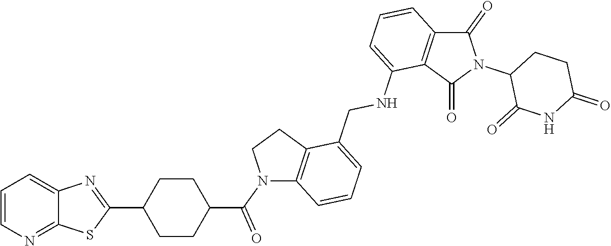 [SMILES: O=C1CCC(N2C(=O)c3cccc(NCc4cccc5c4CCN5C(=O)C4CCC(c5nc6cccnc6s5)CC4)c3C2=O)C(=O)N1] |
| A84 | 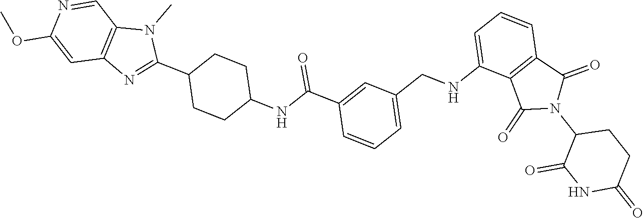 [SMILES: COc1cc2nc(C3CCC(NC(=O)c4cccc(CNc5cccc6c5C(=O)N(C5CCC(=O)NC5=O)C6=O)c4)CC3)n(C)c2cn1] |
| A85 | 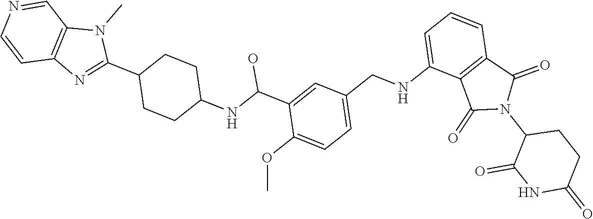 [SMILES: COc1ccc(CNc2cccc3c2C(=O)N(C2CCC(=O)NC2=O)C3=O)cc1C(O)NC1CCC(c2nc3ccncc3n2C)CC1] |
| A86 | 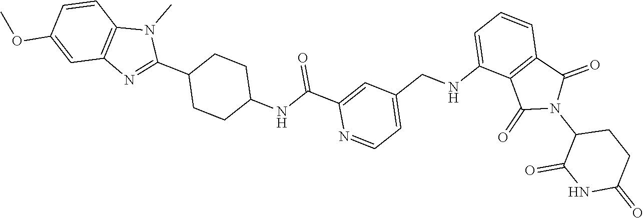 [SMILES: COc1ccc2c(c1)nc(C1CCC(NC(=O)c3cc(CNc4cccc5c4C(=O)N(C4CCC(=O)NC4=O)C5=O)ccn3)CC1)n2C] |
| A87 | 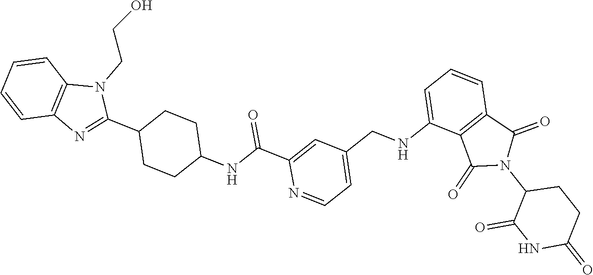 [SMILES: O=C1CCC(N2C(=O)c3cccc(NCc4ccnc(C(=O)NC5CCC(c6nc7ccccc7n6CCO)CC5)c4)c3C2=O)C(=O)N1] |
| A88 | 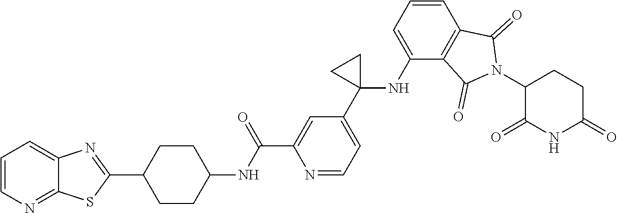 [SMILES: O=C1CCC(N2C(=O)c3cccc(NC4(c5ccnc(C(=O)NC6CCC(c7nc8cccnc8s7)CC6)c5)CC4)c3C2=O)C(=O)N1] |
| A89 | 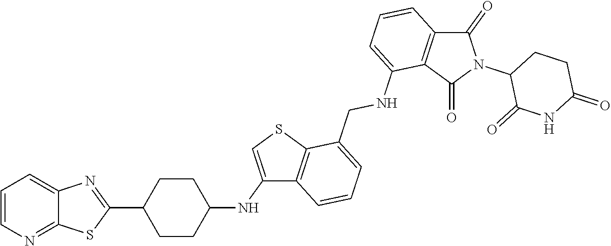 [SMILES: O=C1CCC(N2C(=O)c3cccc(NCc4cccc5c(NC6CCC(c7nc8cccnc8s7)CC6)csc45)c3C2=O)C(=O)N1] |
| A90 | 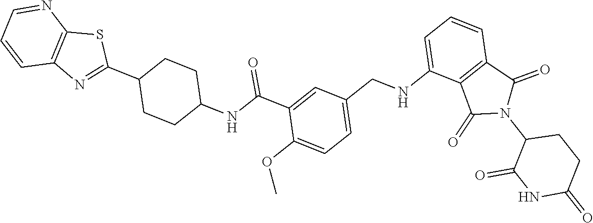 [SMILES: COc1ccc(CNc2cccc3c2C(=O)N(C2CCC(=O)NC2=O)C3=O)cc1C(=O)NC1CCC(c2nc3cccnc3s2)CC1] |
| A91 | 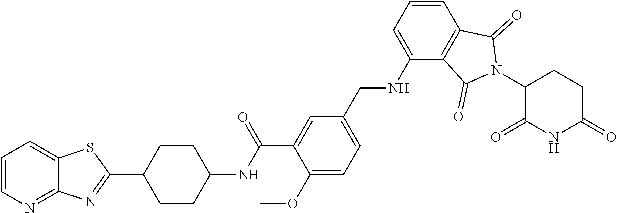 [SMILES: COc1ccc(CNc2cccc3c2C(=O)N(C2CCC(=O)NC2=O)C3=O)cc1C(=O)NC1CCC(c2nc3ncccc3s2)CC1] |
| A92 | 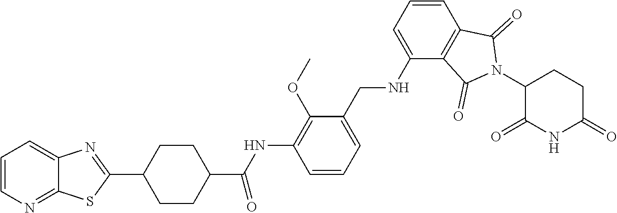 [SMILES: COc1c(CNc2cccc3c2C(=O)N(C2CCC(=O)NC2=O)C3=O)cccc1NC(=O)C1CCC(c2nc3cccnc3s2)CC1] |
| A93 | 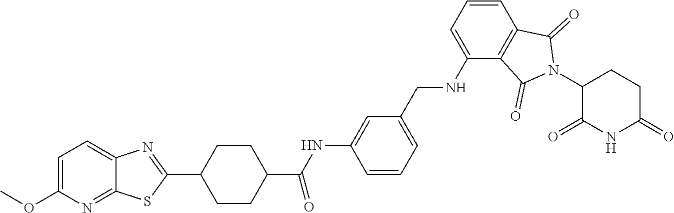 [SMILES: COc1ccc2nc(C3CCC(C(=O)Nc4cccc(CNc5cccc6c5C(=O)N(C5CCC(=O)NC5=O)C6=O)c4)CC3)sc2n1] |
| A94 | 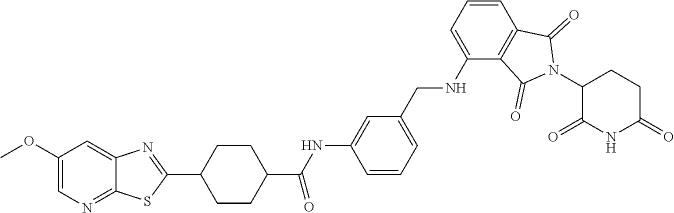 [SMILES: COc1cnc2sc(C3CCC(C(=O)Nc4cccc(CNc5cccc6c5C(=O)N(C5CCC(=O)NC5=O)C6=O)c4)CC3)nc2c1] |
| A95 | 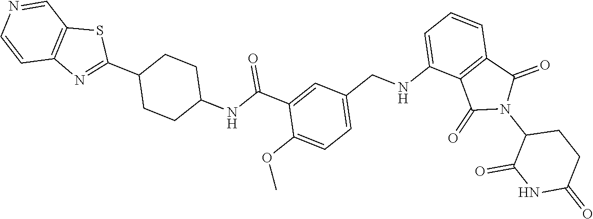 [SMILES: COc1ccc(CNc2cccc3c2C(=O)N(C2CCC(=O)NC2=O)C3=O)cc1C(=O)NC1CCC(c2nc3ccncc3s2)CC1] |
| A96 | 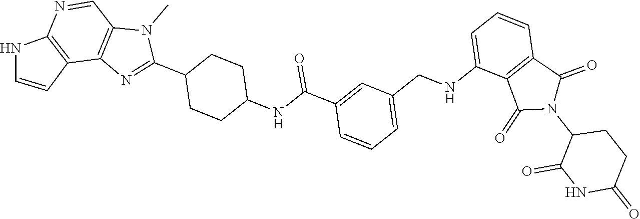 [SMILES: Cn1c(C2CCC(NC(=O)c3cccc(CNc4cccc5c4C(=O)N(C4CCC(=O)NC4=O)C5=O)c3)CC2)nc2c3cc[nH]c3ncc21] |
| A97 | 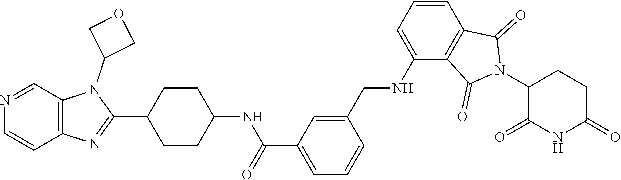 [SMILES: O=C1CCC(N2C(=O)c3cccc(NCc4cccc(C(=O)NC5CCC(c6nc7ccncc7n6C6COC6)CC5)c4)c3C2=O)C(=O)N1] |
| A98 | 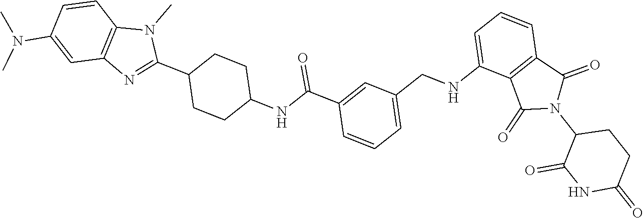 [SMILES: CN(C)c1ccc2c(c1)nc(C1CCC(NC(=O)c3cccc(CNc4cccc5c4C(=O)N(C4CCC(=O)NC4=O)C5=O)c3)CC1)n2C] |
| A99 | 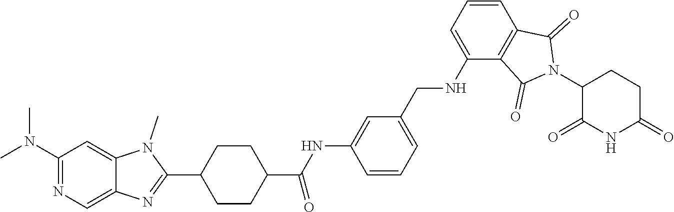 [SMILES: CN(C)c1cc2c(cn1)nc(C1CCC(C(=O)Nc3cccc(CNc4cccc5c4C(=O)N(C4CCC(=O)NC4=O)C5=O)c3)CC1)n2C] |
| A100 | 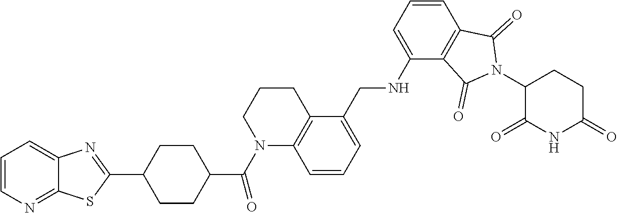 [SMILES: O=C1CCC(N2C(=O)c3cccc(NCc4cccc5c4CCCN5C(=O)C4CCC(c5nc6cccnc6s5)CC4)c3C2=O)C(=O)N1] |
| A101 | 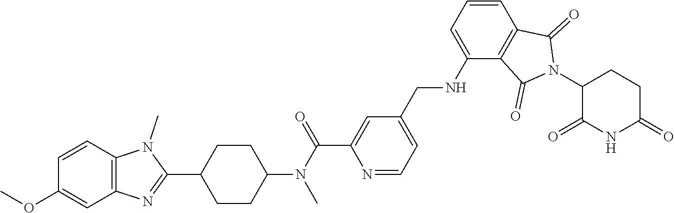 [SMILES: COc1ccc2c(c1)nc(C1CCC(N(C)C(=O)c3cc(CNc4cccc5c4C(=O)N(C4CCC(=O)NC4=O)C5=O)ccn3)CC1)n2C] |
| A102 | 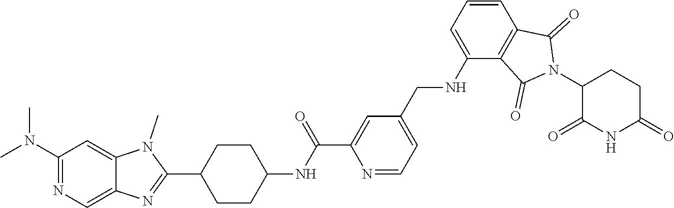 [SMILES: CN(C)c1cc2c(cn1)nc(C1CCC(NC(=O)c3cc(CNc4cccc5c4C(=O)N(C4CCC(=O)NC4=O)C5=O)ccn3)CC1)n2C] |
| A103 | 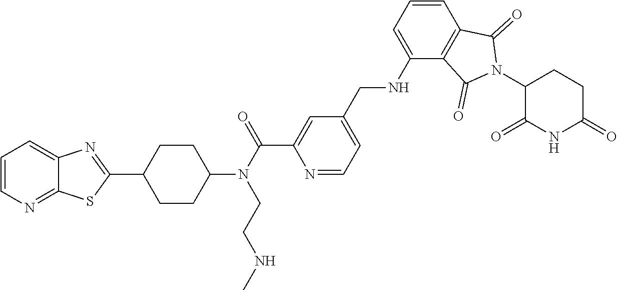 [SMILES: CNCCN(C(=O)c1cc(CNc2cccc3c2C(=O)N(C2CCC(=O)NC2=O)C3=O)ccn1)C1CCC(c2nc3cccnc3s2)CC1] |
| A104 | 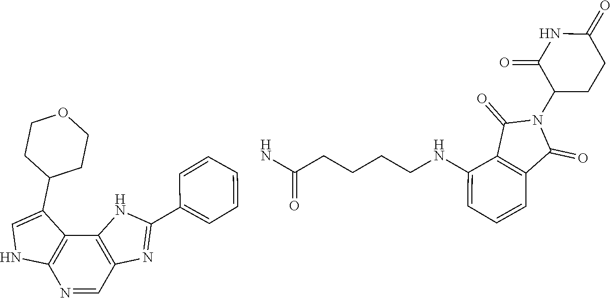 |
| A105 | 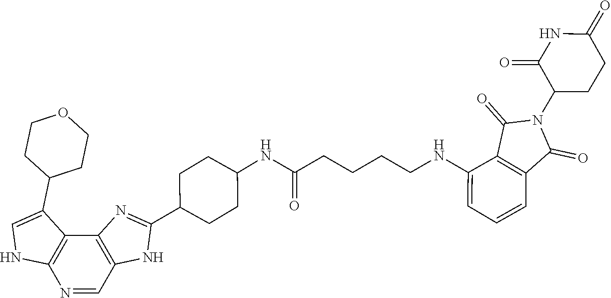 [SMILES: O=C1CCC(N2C(=O)c3cccc(NCCCCC(=O)NC4CCC(c5nc6c(cnc7[nH]cc(C8CCOCC8)c76)[nH]5)CC4)c3C2=O)C(=O)N1] |
| A106 | 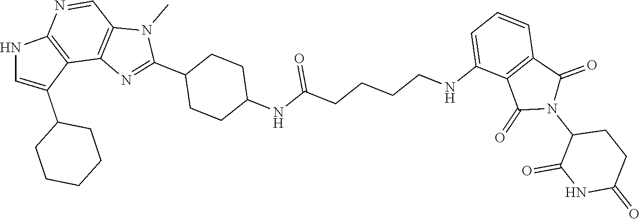 [SMILES: Cn1c(C2CCC(NC(=O)CCCCNc3cccc4c3C(=O)N(C3CCC(=O)NC3=O)C4=O)CC2)nc2c3c(C4CCCCC4)c[nH]c3ncc21] |
| A107 | 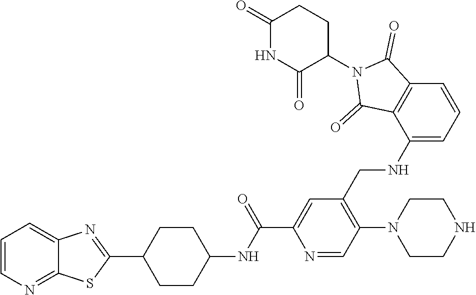 [SMILES: O=C1CCC(N2C(=O)c3cccc(NCc4cc(C(=O)NC5CCC(c6nc7cccnc7s6)CC5)ncc4N4CCNCC4)c3C2=O)C(=O)N1] |
| A108 | 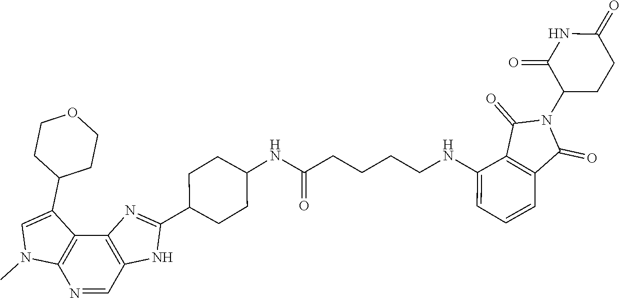 [SMILES: Cn1cc(C2CCOCC2)c2c3nc(C4CCC(NC(=O)CCCCNc5cccc6c5C(=O)N(C5CCC(=O)NC5=O)C6=O)CC4)[nH]c3cnc21] |
| A109 | 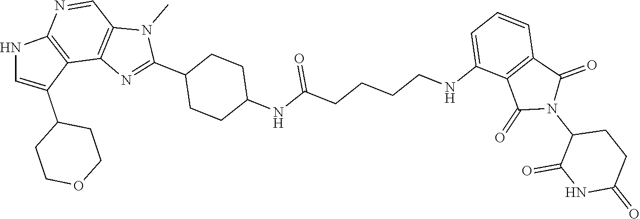 [SMILES: Cn1c(C2CCC(NC(=O)CCCCNc3cccc4c3C(=O)N(C3CCC(=O)NC3=O)C4=O)CC2)nc2c3c(C4CCOCC4)c[nH]c3ncc21] |
| A110 | 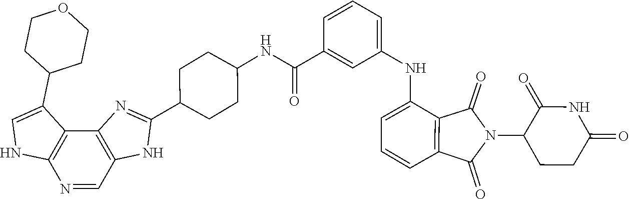 [SMILES: O=C1CCC(N2C(=O)c3cccc(Nc4cccc(C(=O)NC5CCC(c6nc7c(cnc8[nH]cc(C9CCOCC9)c87)[nH]6)CC5)c4)c3C2=O)C(=O)N1] |
| A111 | 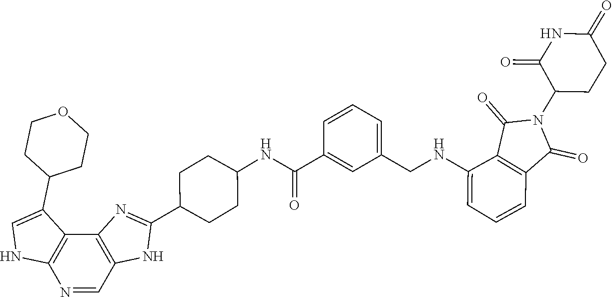 [SMILES: O=C1CCC(N2C(=O)c3cccc(NCc4cccc(C(=O)NC5CCC(c6nc7c(cnc8[nH]cc(C9CCOCC9)c87)[nH]6)CC5)c4)c3C2=O)C(=O)N1] |
| A112 | 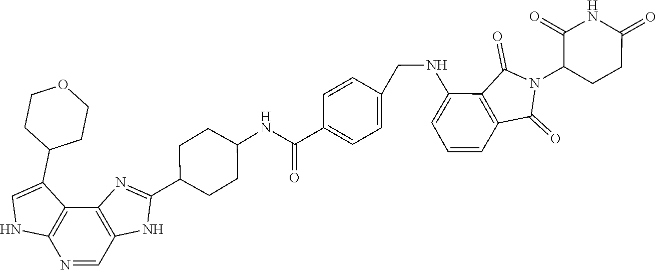 [SMILES: O=C1CCC(N2C(=O)c3cccc(NCc4ccc(C(=O)NC5CCC(c6nc7c(cnc8[nH]cc(C9CCOCC9)c87)[nH]6)CC5)cc4)c3C2=O)C(=O)N1] |
| A113 | 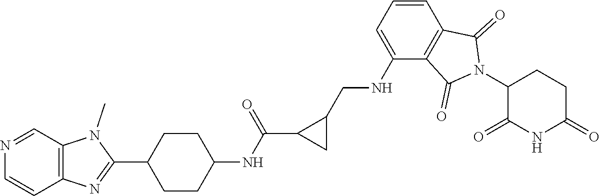 [SMILES: Cn1c(C2CCC(NC(=O)C3CC3CNc3cccc4c3C(=O)N(C3CCC(=O)NC3=O)C4=O)CC2)nc2ccncc21] |
| A114 | 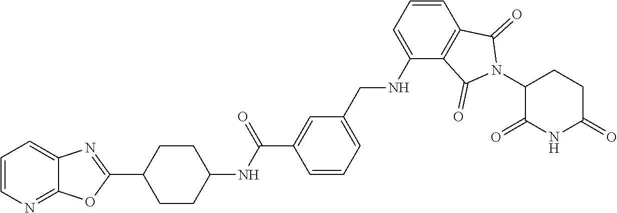 [SMILES: O=C1CCC(N2C(=O)c3cccc(NCc4cccc(C(=O)NC5CCC(c6nc7cccnc7o6)CC5)c4)c3C2=O)C(=O)N1] |
| A115 | 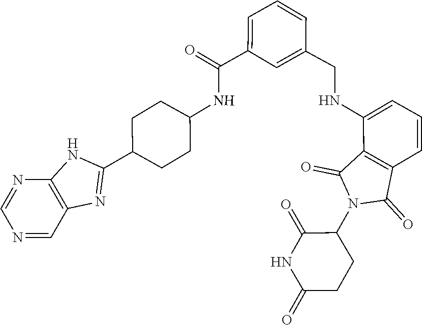 [SMILES: O=C1CCC(N2C(=O)c3cccc(NCc4cccc(C(=O)NC5CCC(c6nc7cncnc7[nH]6)CC5)c4)c3C2=O)C(=O)N1] |
| A116 | 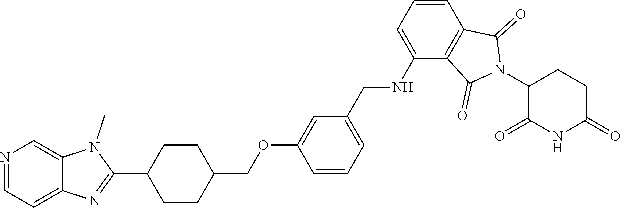 [SMILES: Cn1c(C2CCC(COc3cccc(CNc4cccc5c4C(=O)N(C4CCC(=O)NC4=O)C5=O)c3)CC2)nc2ccncc21] |
| A117 | 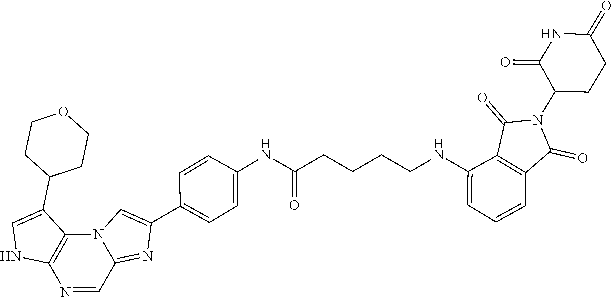 [SMILES: O=C1CCC(N2C(=O)c3cccc(NCCCCC(=O)Nc4ccc(-c5cn6c(cnc7[nH]cc(C8CCOCC8)c76)n5)cc4)c3C2=O)C(=O)N1] |
| A118 | 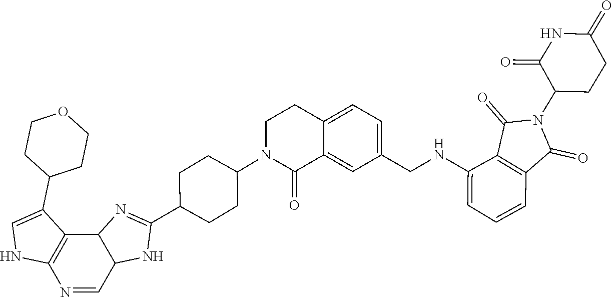 [SMILES: O=C1CCC(N2C(=O)c3cccc(NCc4ccc5c(c4)C(=O)N(C4CCC(C6=NC7c8c(C9CCOCC9)c[nH]c8N=CC7N6)CC4)CC5)c3C2=O)C(=O)N1] |
| A119 | 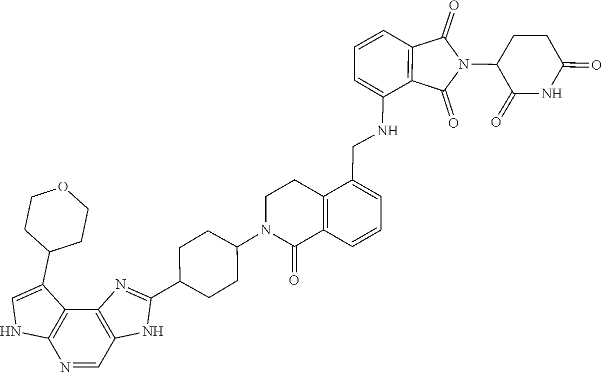 [SMILES: O=C1CCC(N2C(=O)c3cccc(NCc4cccc5c4CCN(C4CCC(c6nc7c(cnc8[nH]cc(C9CCOCC9)c87)[nH]6)CC4)C5=O)c3C2=O)C(=O)N1] |
| A120 | 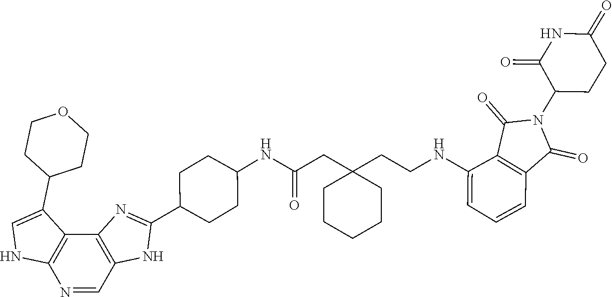 [SMILES: O=C1CCC(N2C(=O)c3cccc(NCCC4(CC(=O)NC5CCC(c6nc7c(cnc8[nH]cc(C9CCOCC9)c87)[nH]6)CC5)CCCCC4)c3C2=O)C(=O)N1] |
| A121 | 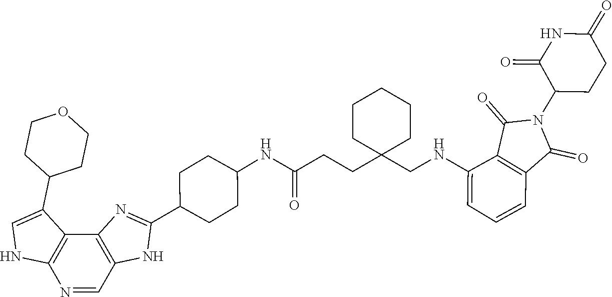 [SMILES: O=C1CCC(N2C(=O)c3cccc(NCC4(CCC(=O)NC5CCC(c6nc7c(cnc8[nH]cc(C9CCOCC9)c87)[nH]6)CC5)CCCCC4)c3C2=O)C(=O)N1] |
| A122 | 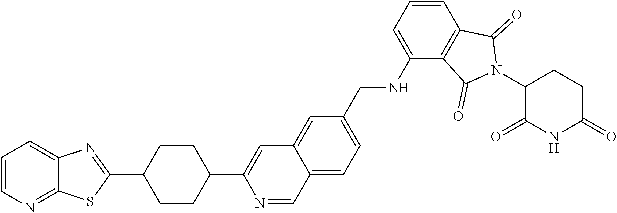 [SMILES: O=C1CCC(N2C(=O)c3cccc(NCc4ccc5cnc(C6CCC(c7nc8cccnc8s7)CC6)cc5c4)c3C2=O)C(=O)N1] |
| A123 | 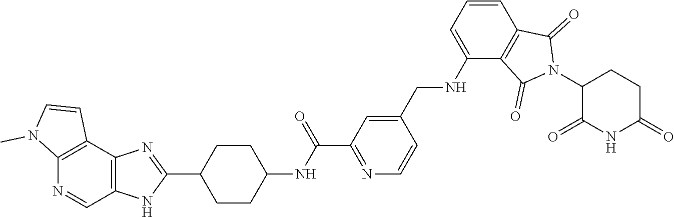 [SMILES: Cn1ccc2c3nc(C4CCC(NC(=O)c5cc(CNc6cccc7c6C(=O)N(C6CCC(=O)NC6=O)C7=O)ccn5)CC4)[nH]c3cnc21] |
| A124 | 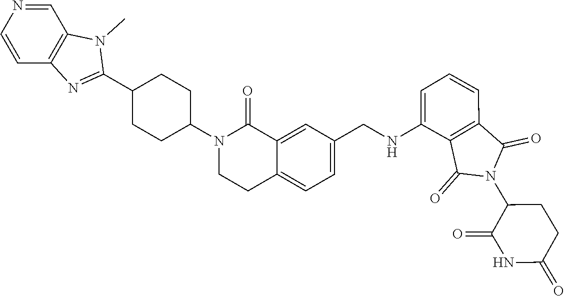 [SMILES: Cn1c(C2CCC(N3CCc4ccc(CNc5cccc6c5C(=O)N(C5CCC(=O)NC5=O)C6=O)cc4C3=O)CC2)nc2ccncc21] |
| A125 | 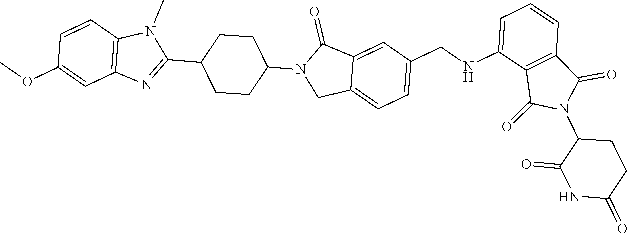 [SMILES: COc1ccc2c(c1)nc(C1CCC(N3Cc4ccc(CNc5cccc6c5C(=O)N(C5CCC(=O)NC5=O)C6=O)cc4C3=O)CC1)n2C] |

In a second aspect of the present invention, provided are a pharmaceutically acceptable salt, a stereoisomer, an ester, a prodrug, a solvate and a deuterated compound of the compound according to the first aspect.

In some embodiments of the present invention, the stereoisomer has the following structures:

| ID | Structure |
|---|---|
| A21′ | 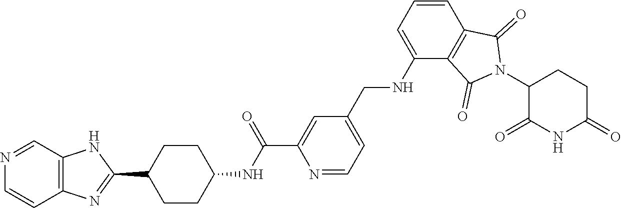 |
| A24′ | 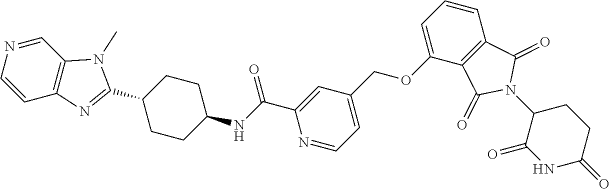 [SMILES: Cn1c2cnccc2nc1[C@H]1CC[C@H](NC(=O)c2cc(COc3cccc4c3C(=O)N(C3CCC(=O)NC3=O)C4=O)ccn2)CC1] |
| A26′ | 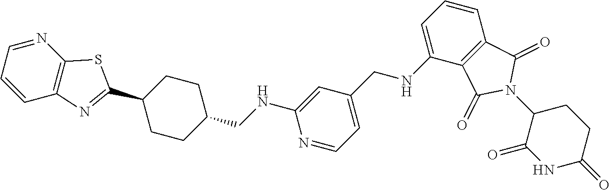 [SMILES: O=C1CCC(N2C(=O)c3cccc(NCc4ccnc(NC[C@H]5CC[C@H](c6nc7cccnc7s6)CC5)c4)c3C2=O)C(=O)N1] |
| A28′ | 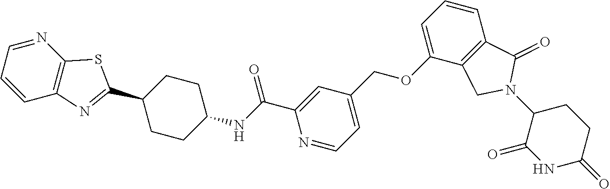 [SMILES: O=C1CCC(N2Cc3c(OCc4ccnc(C(=O)N[C@H]5CC[C@H](c6nc7cccnc7s6)CC5)c4)cccc3C2=O)C(=O)N1] |
| A29′ | 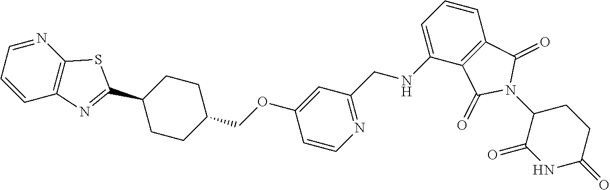 [SMILES: O=C1CCC(N2C(=O)c3cccc(NCc4cc(OC[C@H]5CC[C@H](c6nc7cccnc7s6)CC5)ccn4)c3C2=O)C(=O)N1] |
| A32′ | 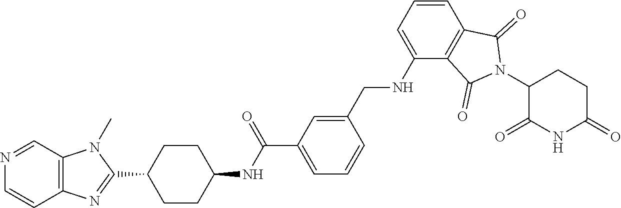 [SMILES: Cn1c2cnccc2nc1[C@H]1CC[C@H](NC(=O)c2cccc(CNc3cccc4c3C(=O)N(C3CCC(=O)NC3=O)C4=O)c2)CC1] |
| A33′ | 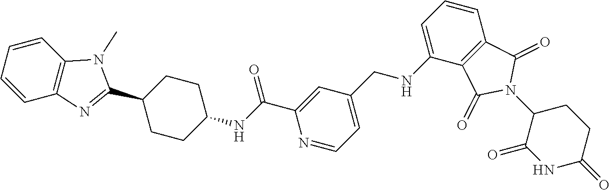 [SMILES: Cn1c2ccccc2nc1[C@H]1CC[C@H](NC(=O)c2cc(CNc3cccc4c3C(=O)N(C3CCC(=O)NC3=O)C4=O)ccn2)CC1] |
| A34′ | 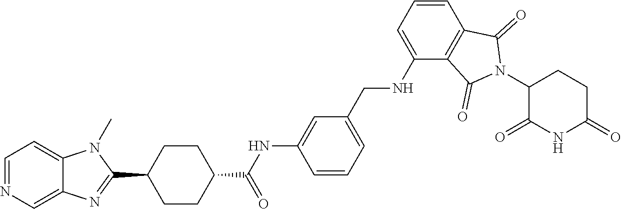 [SMILES: Cn1c2ccncc2nc1[C@H]1CC[C@H](C(=O)Nc2cccc(CNc3cccc4c3C(=O)N(C3CCC(=O)NC3=O)C4=O)c2)CC1] |
| A35′ | 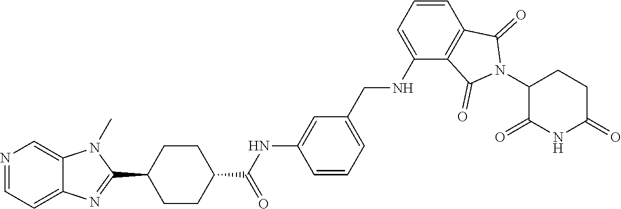 [SMILES: Cn1c2cnccc2nc1[C@H]1CC[C@H](C(=O)Nc2cccc(CNc3cccc4c3C(=O)N(C3CCC(=O)NC3=O)C4=O)c2)CC1] |
| A36′ | 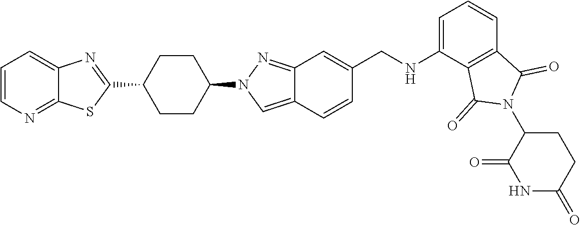 [SMILES: O=C1CCC(N2C(=O)c3cccc(NCc4ccc5cn([C@H]6CC[C@H](c7nc8cccnc8s7)CC6)nc5c4)c3C2=O)C(=O)N1] |
| A37′ | 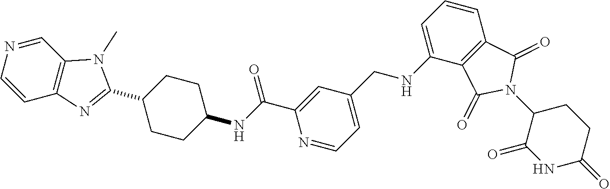 |
| A40′ | 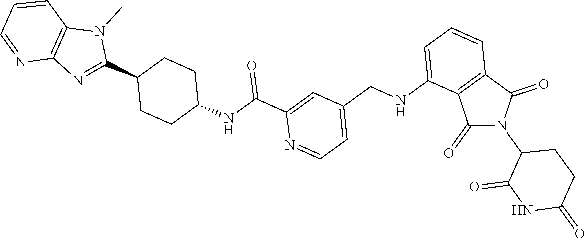 [SMILES: Cn1c2cccnc2nc1[C@H]1CC[C@H](NC(=O)c2cc(CNc3cccc4c3C(=O)N(C3CCC(=O)NC3=O)C4=O)ccn2)CC1] |
| A41′ | 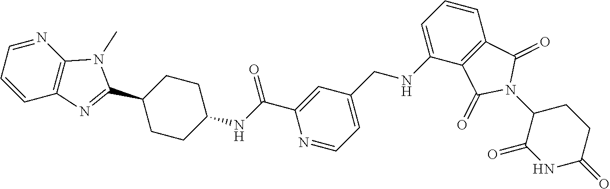 [SMILES: Cn1c2ncccc2nc1[C@H]1CC[C@H](NC(=O)c2cc(CNc3cccc4c3C(=O)N(C3CCC(=O)NC3=O)C4=O)ccn2)CC1] |
| A43′ | 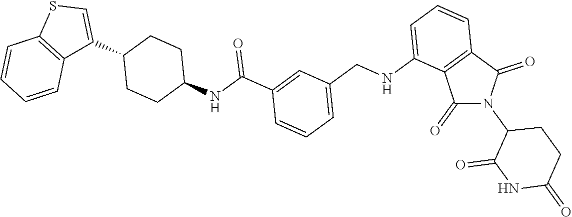 [SMILES: O=C1CCC(N2C(=O)c3cccc(NCc4cccc(C(=O)N[C@H]5CC[C@H](c6csc7ccccc76)CC5)c4)c3C2=O)C(=O)N1] |
| A44′ | 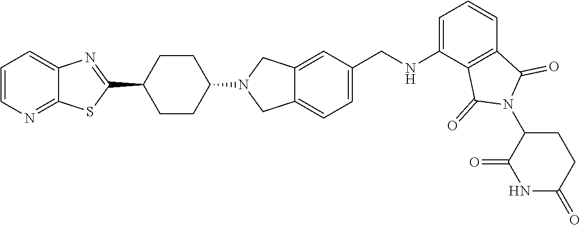 [SMILES: O=C1CCC(N2C(=O)c3cccc(NCc4ccc5c(c4)CN([C@H]4CC[C@H](c6nc7cccnc7s6)CC4)C5)c3C2=O)C(=O)N1] |
| A55′ | 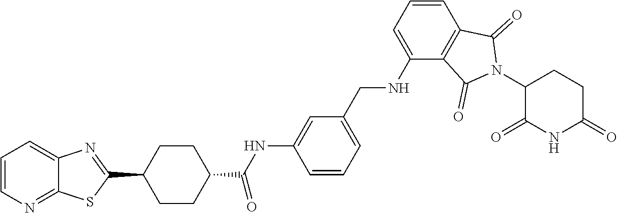 [SMILES: O=C1CCC(N2C(=O)c3cccc(NCc4cccc(NC(=O)[C@H]5CC[C@H](c6nc7cccnc7s6)CC5)c4)c3C2=O)C(=O)N1] |
| A56′ | 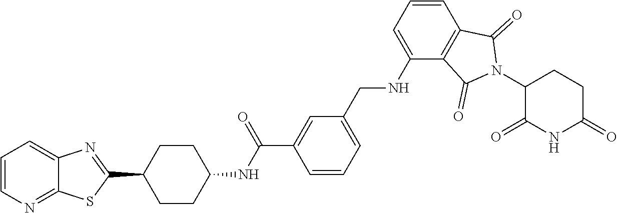 [SMILES: O=C1CCC(N2C(=O)c3cccc(NCc4cccc(C(=O)N[C@H]5CC[C@H](c6nc7cccnc7s6)CC5)c4)c3C2=O)C(=O)N1] |
| A58′ | 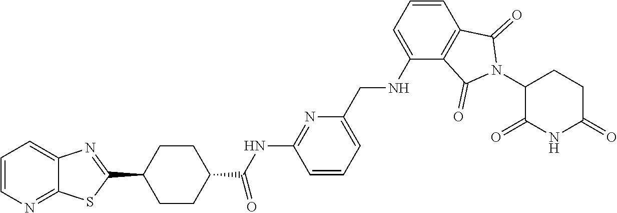 [SMILES: O=C1CCC(N2C(=O)c3cccc(NCc4cccc(NC(=O)[C@H]5CC[C@H](c6nc7cccnc7s6)CC5)n4)c3C2=O)C(=O)N1] |
| A59′ | 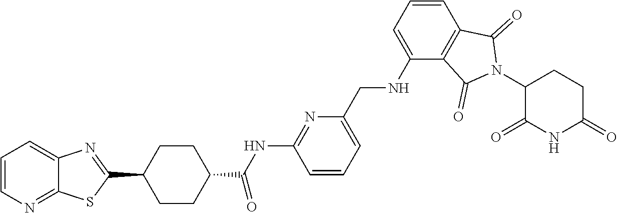 [SMILES: O=C1CCC(N2C(=O)c3cccc(NCc4cccc(NC(=O)[C@H]5CC[C@H](c6nc7cccnc7s6)CC5)n4)c3C2=O)C(=O)N1] |
| A60′ | 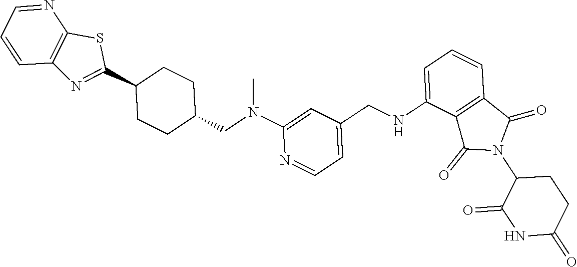 [SMILES: CN(C[C@H]1CC[C@H](c2nc3cccnc3s2)CC1)c1cc(CNc2cccc3c2C(=O)N(C2CCC(=O)NC2=O)C3=O)ccn1] |
| A64′ | 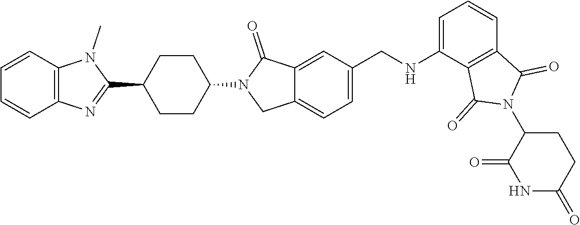 [SMILES: Cn1c2ccccc2nc1[C@H]1CC[C@H](N2Cc3ccc(CNc4cccc5c4C(=O)N(C4CCC(=O)NC4=O)C5=O)cc3C2=O)CC1] |
| A66′ | 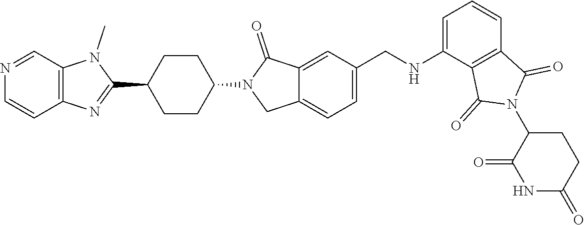 [SMILES: Cn1c2cnccc2nc1[C@H]1CC[C@H](N2Cc3ccc(CNc4cccc5c4C(=O)N(C4CCC(=O)NC4=O)C5=O)cc3C2=O)CC1] |
| A69′ | 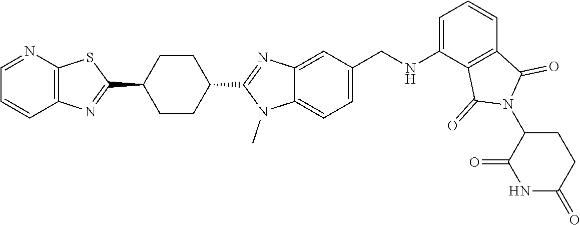 [SMILES: Cn1c2ccc(CNc3cccc4c3C(=O)N(C3CCC(=O)NC3=O)C4=O)cc2nc1[C@H]1CC[C@H](c2nc3cccnc3s2)CC1] |
| A70′ | 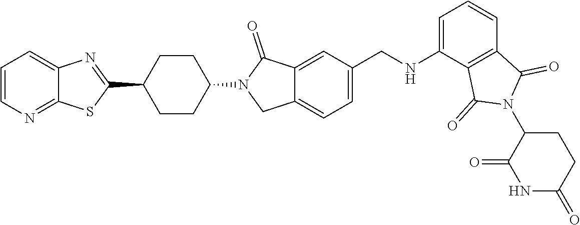 [SMILES: O=C1CCC(N2C(=O)c3cccc(NCc4ccc5c(c4)C(=O)N([C@H]4CC[C@H](c6nc7cccnc7s6)CC4)C5)c3C2=O)C(=O)N1] |
| A71′ | 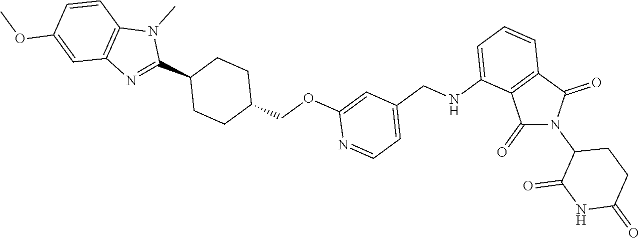 [SMILES: COc1ccc2c(c1)nc([C@H]1CC[C@H](COc3cc(CNc4cccc5c4C(=O)N(C4CCC(=O)NC4=O)C5=O)ccn3)CC1)n2C] |
| A72′ | 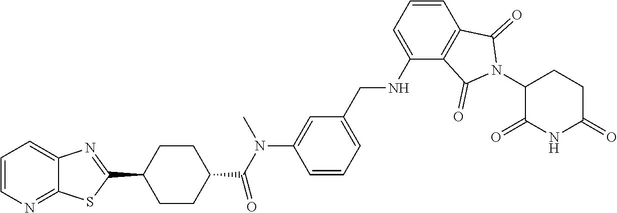 [SMILES: CN(c1cccc(CNc2cccc3c2C(=O)N(C2CCC(=O)NC2=O)C3=O)c1)C(=O)[C@H]1CC[C@H](c2nc3cccnc3s2)CC1] |
| A74′ | 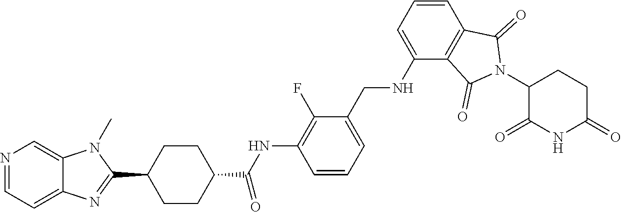 [SMILES: Cn1c2cnccc2nc1[C@H]1CC[C@H](C(=O)Nc2cccc(CNc3cccc4c3C(=O)N(C3CCC(=O)NC3=O)C4=O)c2F)CC1] |
| A75′ | 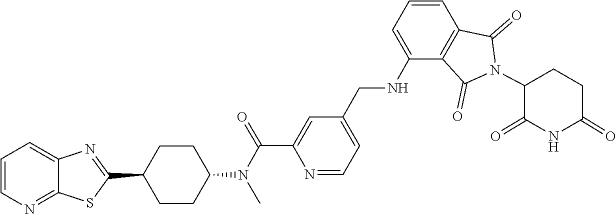 [SMILES: CN(C(=O)c1cc(CNc2cccc3c2C(=O)N(C2CCC(=O)NC2=O)C3=O)ccn1)[C@H]1CC[C@H](c2nc3cccnc3s2)CC1] |
| A76′ | 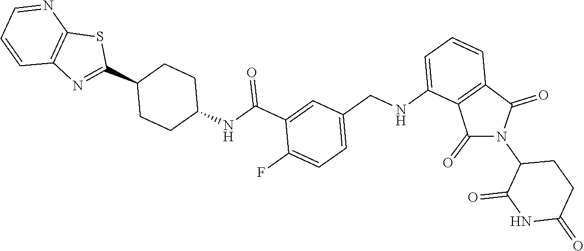 [SMILES: O=C1CCC(N2C(=O)c3cccc(NCc4ccc(F)c(C(=O)N[C@H]5CC[C@H](c6nc7cccnc7s6)CC5)c4)c3C2=O)C(=O)N1] |
| A77′ | 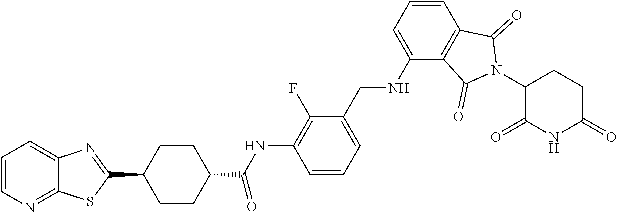 [SMILES: O=C1CCC(N2C(=O)c3cccc(NCc4cccc(NC(=O)[C@H]5CC[C@H](c6nc7cccnc7s6)CC5)c4F)c3C2=O)C(=O)N1] |
| A83′ | 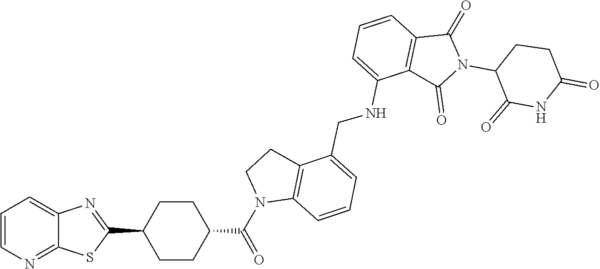 [SMILES: O=C1CCC(N2C(=O)c3cccc(NCc4cccc5c4CCN5C(=O)[C@H]4CC[C@H](c5nc6cccnc6s5)CC4)c3C2=O)C(=O)N1] |
| A85′ | 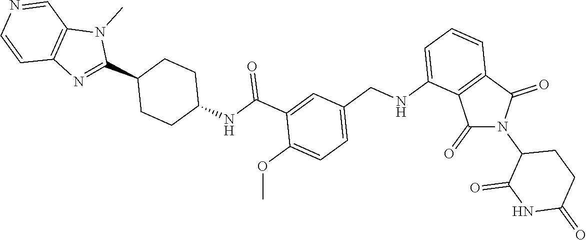 [SMILES: COc1ccc(CNc2cccc3c2C(=O)N(C2CCC(=O)NC2=O)C3=O)cc1C(=O)N[C@H]1CC[C@H](c2nc3ccncc3n2C)CC1] |
| A86′ | 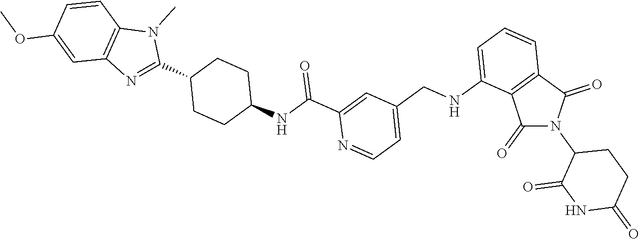 [SMILES: COc1ccc2c(c1)nc([C@H]1CC[C@H](NC(=O)c3cc(CNc4cccc5c4C(=O)N(C4CCC(=O)NC4=O)C5=O)ccn3)CC1)n2C] |
| A87′ | 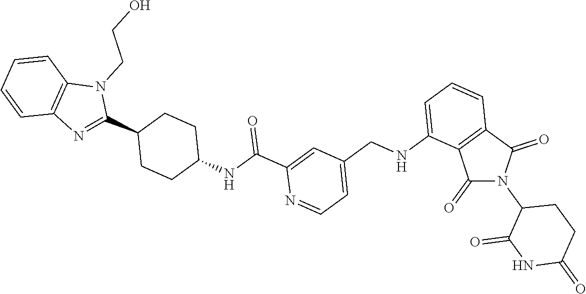 [SMILES: O=C1CCC(N2C(=O)c3cccc(NCc4ccnc(C(=O)N[C@H]5CC[C@H](c6nc7ccccc7n6CCO)CC5)c4)c3C2=O)C(=O)N1] |
| A88′ | 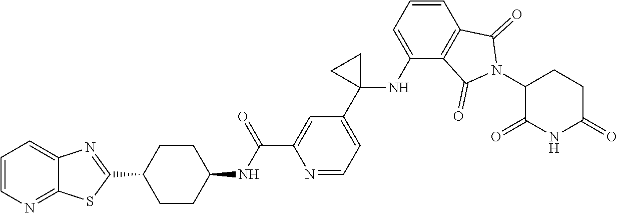 [SMILES: O=C1CCC(N2C(=O)c3cccc(NC4(c5ccnc(C(=O)N[C@H]6CC[C@H](c7nc8cccnc8s7)CC6)c5)CC4)c3C2=O)C(=O)N1] |
| A90′ | 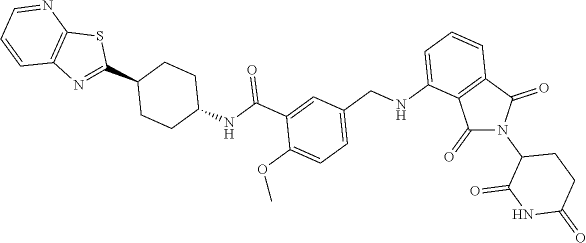 [SMILES: COc1ccc(CNc2cccc3c2C(=O)N(C2CCC(=O)NC2=O)C3=O)cc1C(=O)N[C@H]1CC[C@H](c2nc3cccnc3s2)CC1] |
| A91′ | 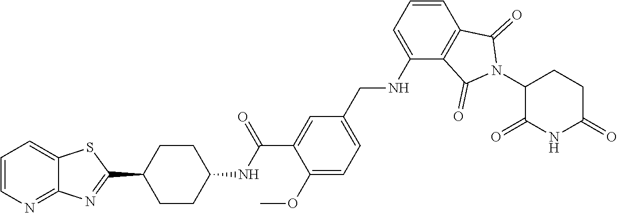 [SMILES: COc1ccc(CNc2cccc3c2C(=O)N(C2CCC(=O)NC2=O)C3=O)cc1C(=O)N[C@H]1CC[C@H](c2nc3ncccc3s2)CC1] |
| A92′ | 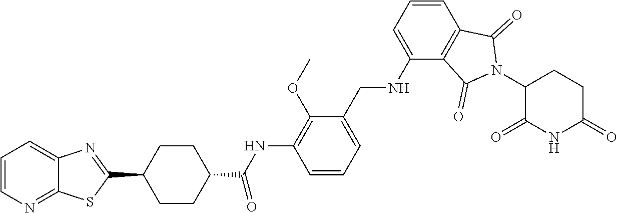 [SMILES: COc1c(CNc2cccc3c2C(=O)N(C2CCC(=O)NC2=O)C3=O)cccc1NC(=O)[C@H]1CC[C@H](c2nc3cccnc3s2)CC1] |
| A93′ | 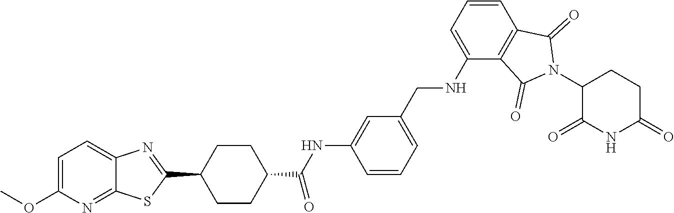 [SMILES: COc1ccc2nc([C@H]3CC[C@H](C(=O)Nc4cccc(CNc5cccc6c5C(=O)N(C5CCC(=O)NC5=O)C6=O)c4)CC3)sc2n1] |
| A94′ | 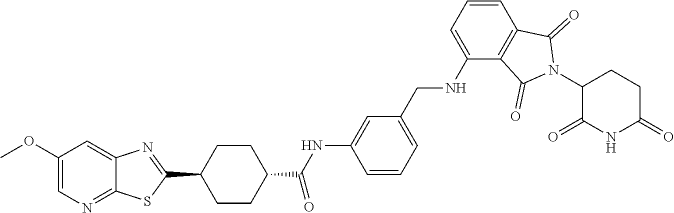 [SMILES: COc1cnc2sc([C@H]3CC[C@H](C(=O)Nc4cccc(CNc5cccc6c5C(=O)N(C5CCC(=O)NC5=O)C6=O)c4)CC3)nc2c1] |
| A95′ | 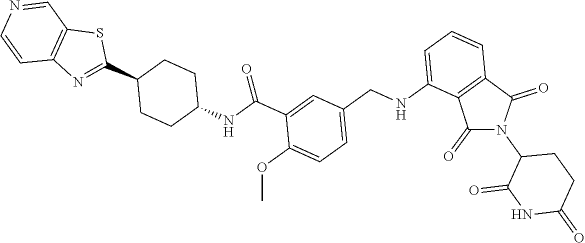 [SMILES: COc1ccc(CNc2cccc3c2C(=O)N(C2CCC(=O)NC2=O)C3=O)cc1C(=O)N[C@H]1CC[C@H](c2nc3ccncc3s2)CC1] |
| A99′ | 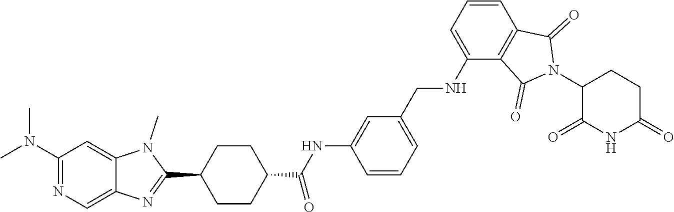 [SMILES: CN(C)c1cc2c(cn1)nc([C@H]1CC[C@H](C(=O)Nc3cccc(CNc4cccc5c4C(=O)N(C4CCC(=O)NC4=O)C5=O)c3)CC1)n2C] |
| A100′ | 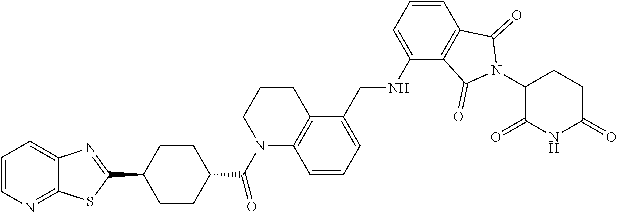 [SMILES: O=C1CCC(N2C(=O)c3cccc(NCc4cccc5c4CCCN5C(=O)[C@H]4CC[C@H](c5nc6cccnc6s5)CC4)c3C2=O)C(=O)N1] |
| A101′ | 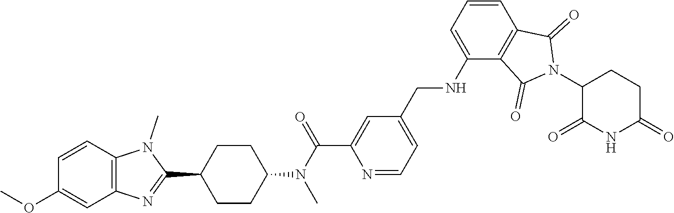 [SMILES: COc1ccc2c(c1)nc([C@H]1CC[C@H](N(C)C(=O)c3cc(CNc4cccc5c4C(=O)N(C4CCC(=O)NC4=O)C5=O)ccn3)CC1)n2C] |
| A102′ | 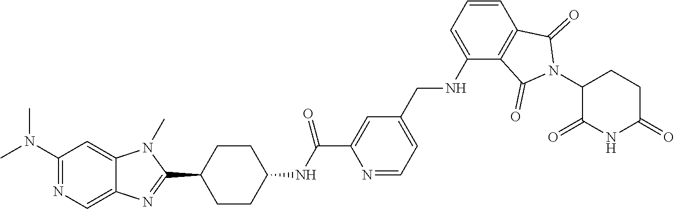 [SMILES: CN(C)c1cc2c(cn1)nc([C@H]1CC[C@H](NC(=O)c3cc(CNc4cccc5c4C(=O)N(C4CCC(=O)NC4=O)C5=O)ccn3)CC1)n2C] |
| A103′ | 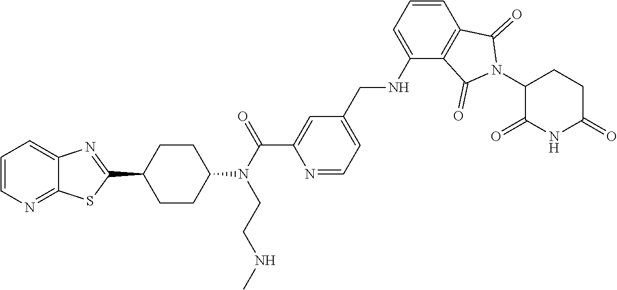 [SMILES: CNCCN(C(=O)c1cc(CNc2cccc3c2C(=O)N(C2CCC(=O)NC2=O)C3=O)ccn1)[C@H]1CC[C@H](c2nc3cccnc3s2)CC1] |
| A107′ | 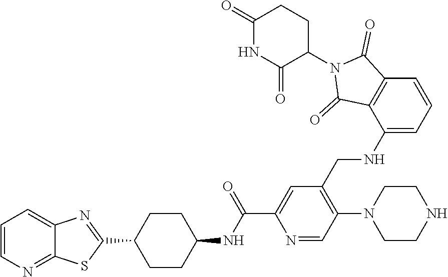 [SMILES: O=C1CCC(N2C(=O)c3cccc(NCc4cc(C(=O)N[C@H]5CC[C@H](c6nc7cccnc7s6)CC5)ncc4N4CCNCC4)c3C2=O)C(=O)N1] |
| A116′ | 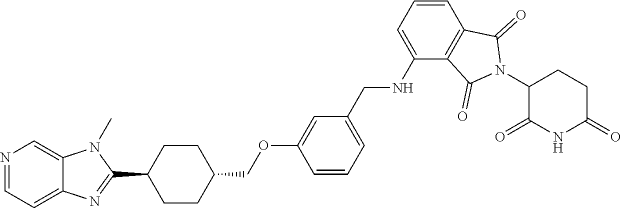 [SMILES: Cn1c2cnccc2nc1[C@H]1CC[C@H](COc2cccc(CNc3cccc4c3C(=O)N(C3CCC(=O)NC3=O)C4=O)c2)CC1] |

In some embodiments, the present disclosure provides a compound selected from the following:

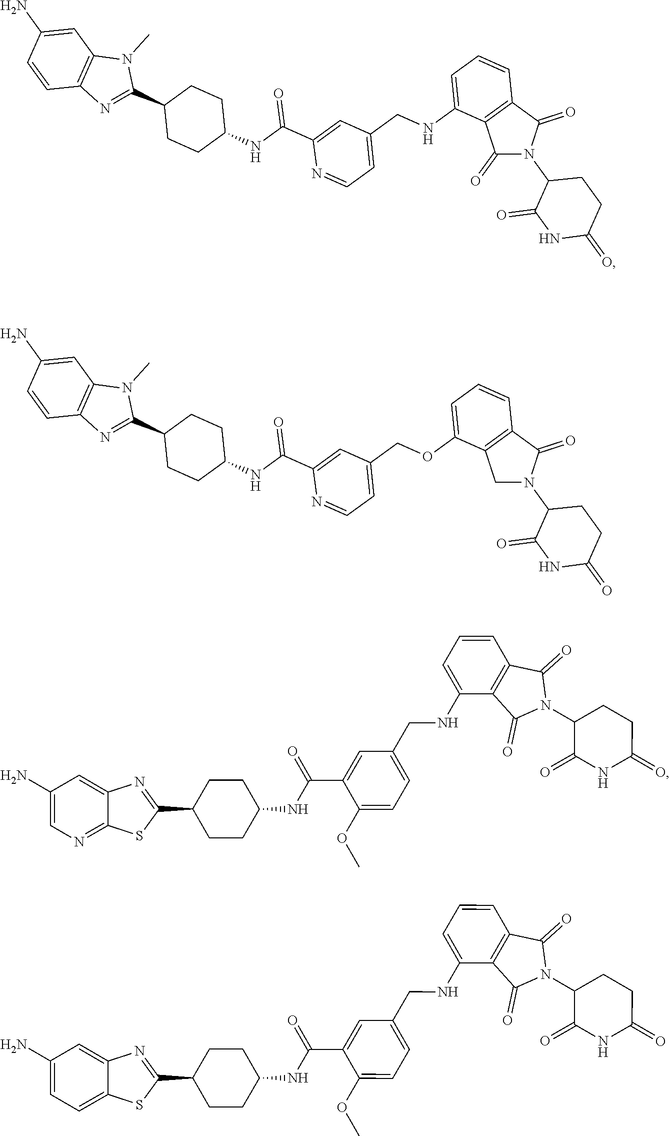 [SMILES: Cn1c2cc(N)ccc2nc1[C@H]1CC[C@H](NC(=O)c2cc(CNc3cccc4c3C(=O)N(C3CCC(=O)NC3=O)C4=O)ccn2)CC1 ; Cn1c2cc(N)ccc2nc1[C@H]1CC[C@H](NC(=O)c2cc(COc3cccc4c3CN(C3CCC(=O)NC3=O)C4=O)ccn2)CC1 ; COc1ccc(CNc2cccc3c2C(=O)N(C2CCC(=O)NC2=O)C3=O)cc1C(=O)N[C@H]1CC[C@H](c2nc3cc(N)cnc3s2)CC1 ; COc1ccc(CNc2cccc3c2C(=O)N(C2CCC(=O)NC2=O)C3=O)cc1C(=O)N[C@H]1CC[C@H](c2nc3cc(N)ccc3s2)CC1]

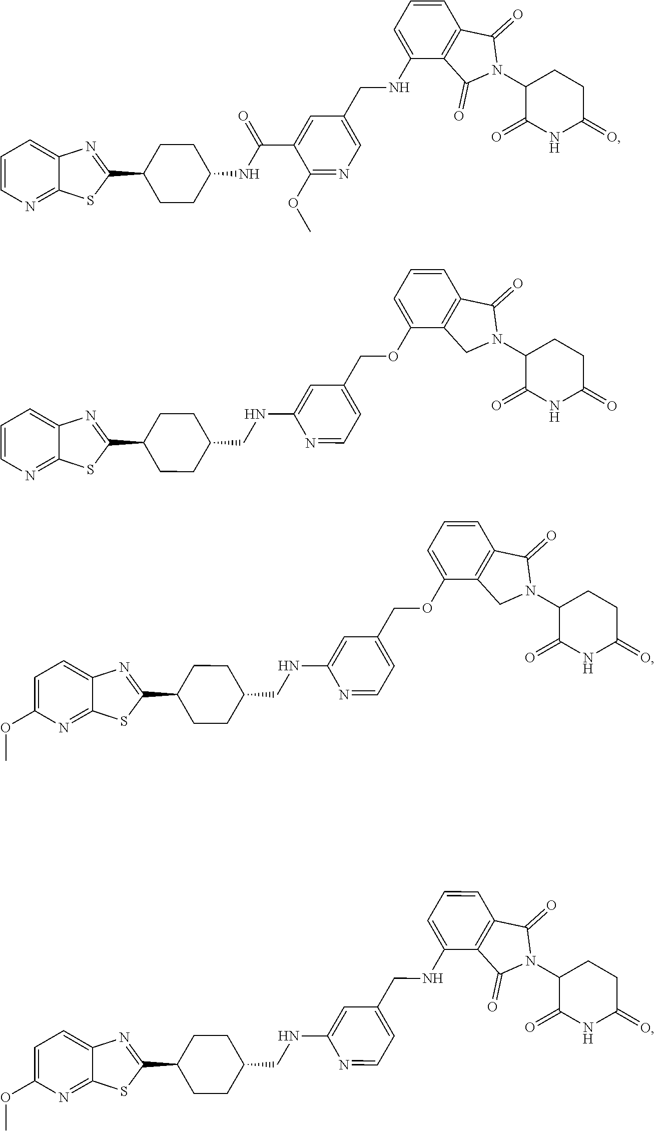 [SMILES: COc1ncc(CNc2cccc3c2C(=O)N(C2CCC(=O)NC2=O)C3=O)cc1C(=O)N[C@H]1CC[C@H](c2nc3cccnc3s2)CC1 ; O=C1CCC(N2Cc3c(OCc4ccnc(NC[C@H]5CC[C@H](c6nc7cccnc7s6)CC5)c4)cccc3C2=O)C(=O)N1 ; COc1ccc2nc([C@H]3CC[C@H](CNc4cc(COc5cccc6c5CN(C5CCC(=O)NC5=O)C6=O)ccn4)CC3)sc2n1 ; COc1ccc2nc([C@H]3CC[C@H](CNc4cc(CNc5cccc6c5C(=O)N(C5CCC(=O)NC5=O)C6=O)ccn4)CC3)sc2n1]

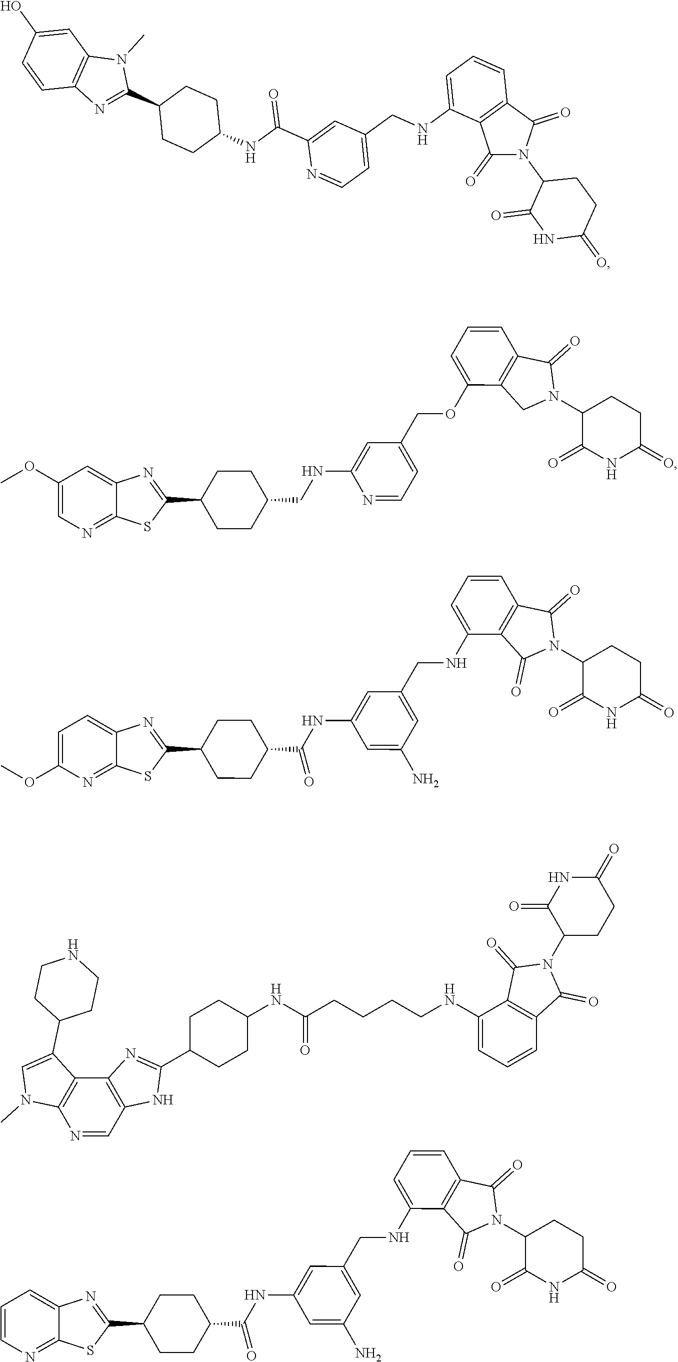 [SMILES: Cn1c2cc(O)ccc2nc1[C@H]1CC[C@H](NC(=O)c2cc(CNc3cccc4c3C(=O)N(C3CCC(=O)NC3=O)C4=O)ccn2)CC1 ; COc1cnc2sc([C@H]3CC[C@H](CNc4cc(COc5cccc6c5CN(C5CCC(=O)NC5=O)C6=O)ccn4)CC3)nc2c1 ; COc1ccc2nc([C@H]3CC[C@H](C(=O)Nc4cc(N)cc(CNc5cccc6c5C(=O)N(C5CCC(=O)NC5=O)C6=O)c4)CC3)sc2n1 ; Cn1cc(C2CCNCC2)c2c3nc(C4CCC(NC(=O)CCCCNc5cccc6c5C(=O)N(C5CCC(=O)NC5=O)C6=O)CC4)[nH]c3cnc21 ; Nc1cc(CNc2cccc3c2C(=O)N(C2CCC(=O)NC2=O)C3=O)cc(NC(=O)[C@H]2CC[C@H](c3nc4cccnc4s3)CC2)c1]

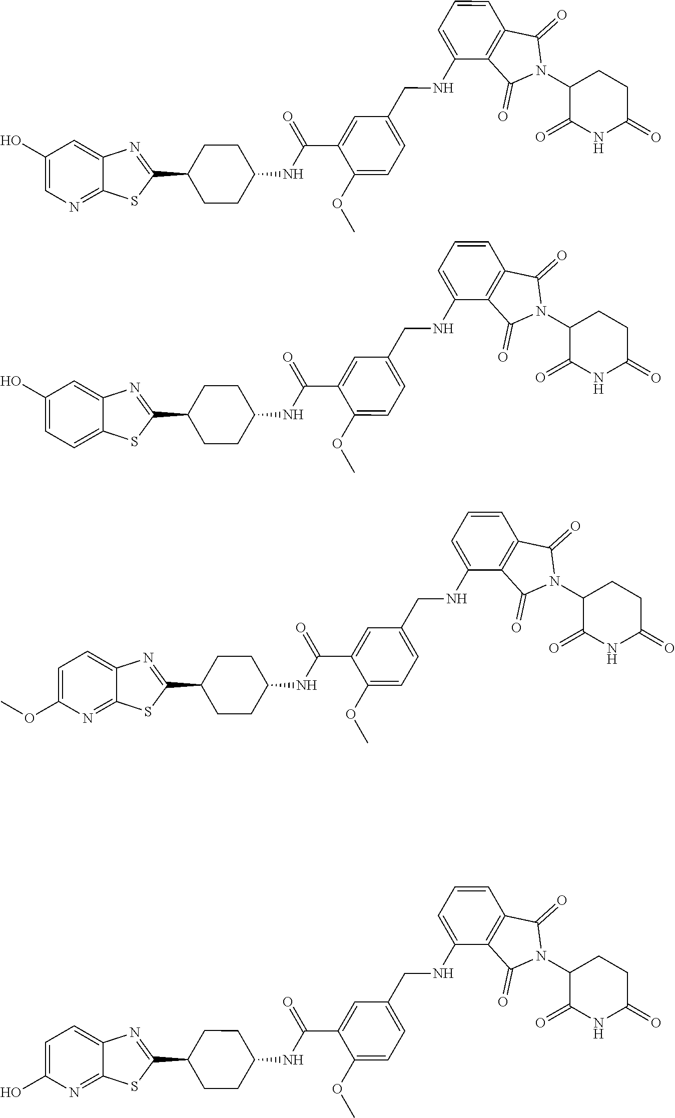 [SMILES: COc1ccc(CNc2cccc3c2C(=O)N(C2CCC(=O)NC2=O)C3=O)cc1C(=O)N[C@H]1CC[C@H](c2nc3cc(O)cnc3s2)CC1 ; COc1ccc(CNc2cccc3c2C(=O)N(C2CCC(=O)NC2=O)C3=O)cc1C(=O)N[C@H]1CC[C@H](c2nc3cc(O)ccc3s2)CC1 ; COc1ccc2nc([C@H]3CC[C@H](NC(=O)c4cc(CNc5cccc6c5C(=O)N(C5CCC(=O)NC5=O)C6=O)ccc4OC)CC3)sc2n1 ; COc1ccc(CNc2cccc3c2C(=O)N(C2CCC(=O)NC2=O)C3=O)cc1C(=O)N[C@H]1CC[C@H](c2nc3ccc(O)nc3s2)CC1]

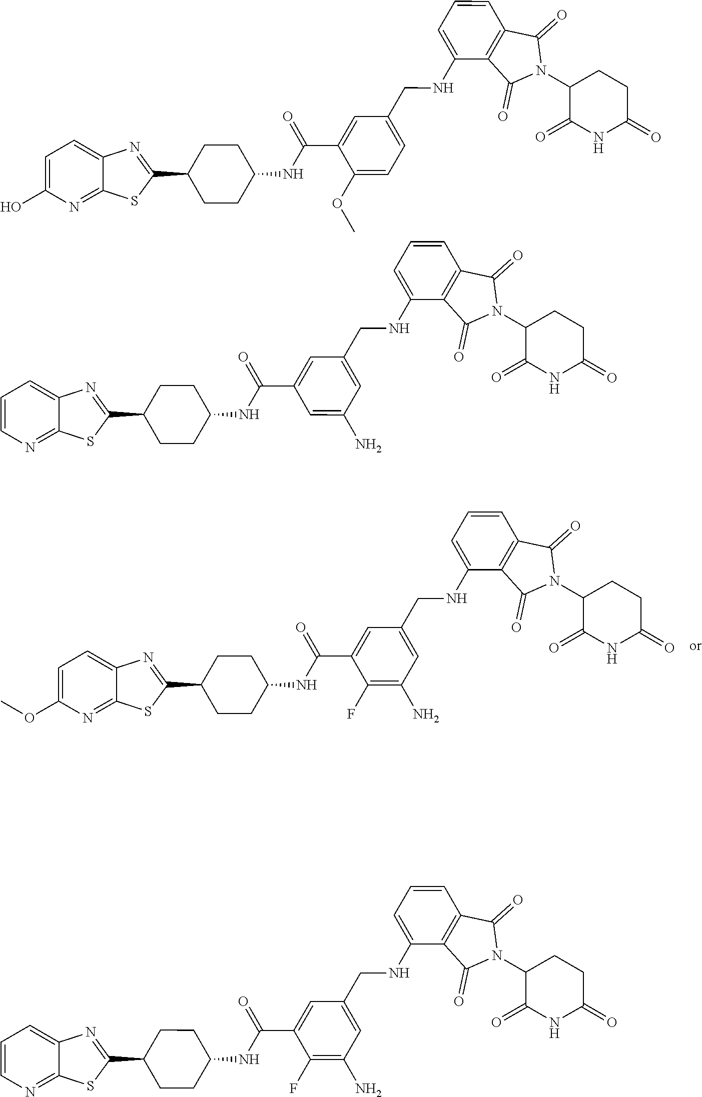 [SMILES: COc1ccc(CNc2cccc3c2C(=O)N(C2CCC(=O)NC2=O)C3=O)cc1C(=O)N[C@H]1CC[C@H](c2nc3ccc(O)nc3s2)CC1 ; Nc1cc(CNc2cccc3c2C(=O)N(C2CCC(=O)NC2=O)C3=O)cc(C(=O)N[C@H]2CC[C@H](c3nc4cccnc4s3)CC2)c1 ; COc1ccc2nc([C@H]3CC[C@H](NC(=O)c4cc(CNc5cccc6c5C(=O)N(C5CCC(=O)NC5=O)C6=O)cc(N)c4F)CC3)sc2n1 ; Nc1cc(CNc2cccc3c2C(=O)N(C2CCC(=O)NC2=O)C3=O)cc(C(=O)N[C@H]2CC[C@H](c3nc4cccnc4s3)CC2)c1F]

or a pharmaceutically acceptable salt thereof.

In a third aspect of the present invention, provided is a pharmaceutical composition comprising the compound according to the first aspect or a pharmaceutically acceptable salt, a stereoisomer, an ester, a prodrug, a solvate and a deuterated compound thereof.

Specifically, the pharmaceutical composition further comprises one or more pharmaceutically acceptable excipients.

Specifically, the pharmaceutically acceptable excipients may be selected from: a filler, a binder, a lubricant, and the like.

Specifically, in the pharmaceutical composition, the pharmaceutically acceptable salt, the stereoisomer, the ester, the prodrug, the solvate and the deuterated compound of the compound according to the first aspect can be used alone or in combination with other kinds of active ingredients.

Specifically, the pharmaceutical composition may also comprise an antioxidant, a buffer, a bacteriostat, a solute which renders a formulation isotonic with the blood of a subject, and aqueous and non-aqueous sterile suspensions which may comprise suspending agents, solubilizers, thickening agents, stabilizers, preservatives, etc.

Specifically, the pharmaceutical composition is suitable for being administered enterally or parenterally, e.g., by intravenous, intramuscular, intradermal and subcutaneous routes.

Specifically, the pharmaceutical composition can be prepared as pharmaceutical formulations in the form of: injections, syrups, elixirs, suspensions, powders, granules, tablets, capsules, lozenges, creams, ointments, lotions, gels, emulsions and the like.

In the preparation of injections, any carrier commonly used in the art can be used, for example, water, ethanol, propylene glycol, ethoxylated isostearyl alcohol, polyethoxylated isostearyl alcohol, and fatty acid esters of polyethylene sorbitan. In addition, conventional solvents and buffers can be added.

Specifically, the pharmaceutical composition is preferably in unit dosage form. In this form, the formulation is subdivided into unit dosages containing an appropriate amount of active components. The unit dosage form can be capsules, tablets or any other dosage forms; in addition, the unit dosage form can be packaged formulations such as tablets, capsules and powders packaged in vials or ampoules.

In a fourth aspect of the present invention, provided is use of the compound according to the first aspect, or a pharmaceutically acceptable salt, a stereoisomer, an ester, a prodrug, a solvate and a deuterated compound thereof, in preparing a medicament for preventing and/or treating a GSPT1-associated disease.

Specifically, the GSPT1-associated disease is selected from: an autoimmune disease, an inflammatory disease, a heteroimmune disease, a neurodegenerative disease, and a tumor.

Specifically, the autoimmune disease described above includes, but is not limited to, one or more of organ-specific autoimmune diseases, systemic lupus erythematosus, rheumatoid arthritis, systemic vasculitis, scleroderma, pemphigus, dermatomyositis, mixed connective tissue disease, autoimmune hemolytic anemia, thyroid autoimmune disease and ulcerative colitis.

Specifically, the inflammatory disease described above includes, but is not limited to, one or more of osteoarthritis, gout, chronic obstructive pulmonary disease, periodic fever, rash, lymphadenectasis, sepsis, osteoarthritis, ankylosing spondylitis, psoriasis, inflammatory bowel disease, Crohn's disease, ulcerative colitis, uveitis, asthma, and allergy.

Specifically, the heteroimmune disease is transplant rejection after organ transplantation.

Specifically, the neurodegenerative disease described above includes, but is not limited to Alzheimer's disease(AD), Parkinson's disease (PD), Huntington's disease (HD), amyotrophic lateral sclerosis (ALS), different types of spinocerebellar ataxia (SCA), Pick's disease, etc.

Specifically, the tumor described above includes, but is not limited to adrenal cancer, anal cancer, angiosarcoma, appendiceal cancer, biliary tract cancer, bladder cancer, breast cancer, brain cancer, bronchial cancer, carcinoid tumor, cervical cancer, choriocarcinoma, chordoma, craniopharyngioma, colorectal cancer, connective tissue cancer, epithelial cancer, ependymoma, endothelial sarcoma, endometrial cancer, esophageal cancer, Ewing sarcoma, eye cancer, gallbladder cancer, gastric cancer, gastrointestinal stromal tumor (GIST), germ cell cancer, head and neck cancer, hematologic malignancies, hemangioblastoma, hypopharynx cancer, inflammatory myofibroblastoma, immune cell amyloidosis, kidney cancer, liver cancer, lung cancer, leiomyosarcoma (LMS), muscle cancer, mesothelioma, myeloproliferative disease (MPD), neuroblastoma, neurofibroma, neuroendocrine cancer, osteosarcoma, ovarian cancer, papillary adenocarcinoma, pancreatic cancer, penile cancer, pineal tumor, primitive neuroectodermal tumor (PNT), prostate cancer, rhabdomyosarcoma, salivary gland cancer, skin cancer, small bowel cancer, soft tissue sarcoma, sebaceous gland cancer, sweat gland cancer, synovial tumor, testicular cancer, thyroid cancer, urethral cancer, vaginal cancer, and vulval cancer.

More specifically, the tumor described above is a hematologic malignancy, e.g., leukemia, lymphoma, or multiple myeloma (MM).

Specifically, the leukemia may be chronic lymphocytic leukemia (CLL)(e.g., B cell CLL, or T cell CLL), chronic myelogenous leukemia (CML)(e.g., B cell CML, or T cell CML), acute lymphocytic leukemia (ALL)(e.g., B cell ALL, or T cell ALL), acute myelogenous leukemia (AML)(e.g., B cell AML, or T cell AML), or acute monocytic leukemia, particularly acute myelogenous leukemia. Specifically, leukemia may be recurrent, refractory or drug-resistant.

Specifically, the lymphoma may be Hodgkin lymphoma (HL)(e.g., B-cell HL, or T-cell HL), non-Hodgkin lymphoma (NHL)(e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, or splenic marginal zone B-cell lymphoma), primary mediastinal B-cell lymphoma, Burkitt lymphoma, or lymphoplasmacytic lymphoma (i.e., Waldenström's macroglobulinemia)), hairy cell leukemia (HCL), immunoblastic large cell lymphoma, precursor B-lymphoblastic lymphoma, primary central nervous system (CNS) lymphoma, T-cell NHL such as precursor T-lymphoblastic lymphoma/leukemia, peripheral T-cell lymphoma (PTCL)(e.g., cutaneous T-cell lymphoma (CTCL)(e.g., mycosis fungoides, or Sezary syndrome)), angioimmunoblastic T-cell lymphoma, extranodal natural killer T-cell lymphoma, enteropathy-associated T-cell lymphoma, subcutaneous panniculitis-like T-cell lymphoma and anaplastic large-cell lymphoma, or NK/T-cell lymphoma, particularly diffuse large B-cell lymphoma (DLBCL).

More specifically, the tumor described above is multiple myeloma (MM), diffuse large B-cell lymphoma (DLBCL), or acute monocytic leukemia.

In a fifth aspect of the present invention, provided is a method for preventing and/or treating a GSPT1-associated disease comprising a step of administering to a subject in need the compound according to the first aspect of the present invention or a pharmaceutically acceptable salt, a stereoisomer, an ester, a prodrug, a solvate and a deuterated compound thereof, or the pharmaceutical composition according to the third aspect of the present invention.

Specifically, in the method described above, the GSPT1-associated disease has the corresponding definition as described in the fourth aspect of the present invention.

Specifically, the subject described above may be a mammal, particularly a human.

### DETAILED DESCRIPTION

The compounds of the present disclosure can be generally characterized as having a formula according to any of Formula (I)(including its subformulae such as Formulae I-1 to I-8, (II), (III), (IV), or (V)), Formula A, Formula B, Formula C, and their subformulae. Compounds of Formula (I) and its subformulae are described in detail in the Summary section.

In some embodiments, the present disclosure provides a compound of Formula A as defined in any of the enumerated Embodiments 1-31. In some embodiments, the present disclosure provides a compound of Formula B as defined in any of the enumerated Embodiments 32-39. In some embodiments, the present disclosure provides a compound of Formula C as defined in any of the enumerated Embodiments 40-49. In some embodiments, the variables for Formula A, B, or C can have any of the respective definitions of those specific compounds described herein, such as the respective definitions from those specific compounds selected from Examples 1-150.

### Formula A

In embodiments according to Formula A, T¹ is a glutarimide containing moiety, which typically can bind with a cereblon pocket in an E3 ligase. A glutarimide containing moiety as used herein refers to a moiety that derives from glutarimide,

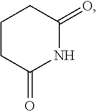

which is optionally substituted. For example, in some typical embodiments, T¹ has a structure according to

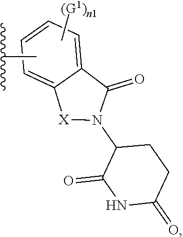

wherein:
X is CH₂ or C(═O),
n1 is an integer of 0-2, and
G¹ at each occurrence is independently halogen, CN, OH, NH₂, an optionally substituted C₁₋₆ alkyl, optionally substituted C₁₋₆ heteroalkyl, optionally substituted 3-8 membered carbocyclic or heterocyclic ring, optionally substituted phenyl, or optionally substituted heteroaryl. In some embodiments, n1 is 0. In some embodiments, n1 is 1 and G¹ can be halogen (e.g., F), C₁₋₄ alkyl optionally substituted with F, or C₁₋₄ heteroalkyl (e.g., C₁₋₄ alkoxy or C₁₋₄ monoalkylamino or dialkylamino) optionally substituted with F. In some embodiments, X is C(O). In some embodiments, X is CH₂. The remainder of the molecule can attach to T¹ at any position of the benzene ring. However, in some preferred embodiments, the compound of Formula A can be characterized as having a structure according to Formula A1:

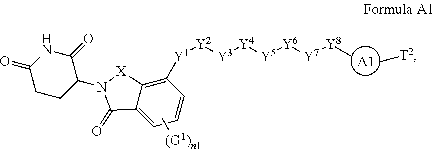

wherein the variables are defined herein.

In some embodiments according to Formula A (e.g., A1), Y¹ to Y⁷ represent a chain structure, for example, in some embodiments, Y¹ to Y⁷ can represent a heteroalkylene chain having 7 chain forming atoms, such as

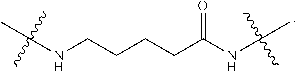 [SMILES: *NCCCCC(=O)N*]

In some embodiments, Y¹ to Y can have any of the definitions according to enumerated Embodiments 5, 6, 12-14, and 16-18, respectively.

In some embodiments according to Formula A (e.g., A1), Y¹ to Y⁷ represent a ring-chain structure, in other words, at least some of Y¹ to Y⁷ are represented by a ring structure and at least one of Y¹ to Y⁷ is considered as having a chain atom. The ring-chain structure is not particularly limited.

For example, in some embodiments, Y³, Y⁴, and Y⁵ together represent an optionally substituted 3-8 membered ring, and Y¹, Y², Y⁶, and Y⁷ are as defined herein, e.g., in enumerated Embodiment 1-(1). In some embodiments, Y¹, Y², Y⁶, and Y⁷ can be any of those defined in enumerated Embodiments 5, 6, and 16-18, respectively. In some preferred embodiments, Y¹-Y² can be a NHCH₂ or OCH₂ linker, and Y⁶—Y⁷ can be —C(O)NH—, —C(O)—N(CH₃)—, —NHCH₂—, or —OCH₂—. It should be noted that these bivalent linkers are not limited to any particular direction and can attach to the remainder of the molecule through either directions (left to right or right to left). For example, in some embodiments, the compound of Formula A can be characterized as having a structure according to Formula A1a, A1b, A1c, A1d, A1e, A1f, A1g, or A1h:

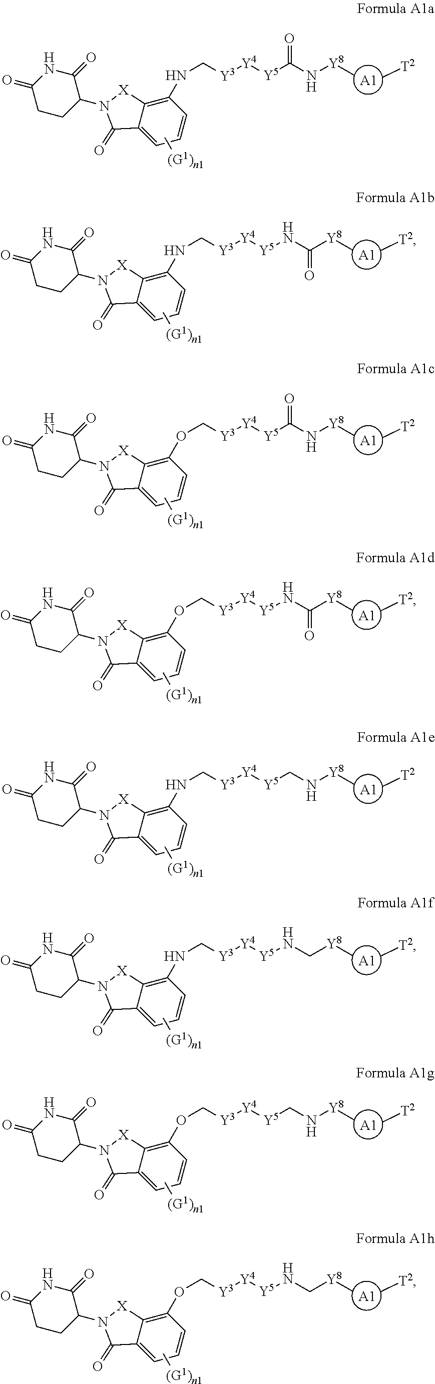

wherein the variables are defined herein.

In some embodiments according to Formula A (e.g., Formula A1a, A1b, A1c, A1d, A1e, A1f, A1g, or A1h), Y³, Y⁴, and Y⁵ together represent an optionally substituted phenylene or an optionally substituted 5 or 6-membered heteroarylene (e.g., as defined in any of Embodiments 8-10). For example, in some embodiments, Y³, Y⁴, and Y⁵ together represent an optionally substituted 1,3-phenylene, i.e., Y² and Y⁶ are meta to each other, such as shown in Formula A1i:

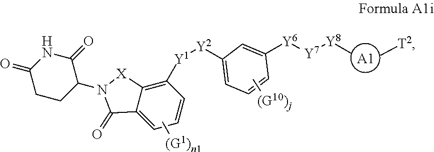

wherein j is 0, 1, or 2, and G¹⁰ at each occurrence is halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl. In some embodiments, G¹⁰ at each occurrence is halogen, OH, NH₂, CN, C₁₋₄ alkyl optionally substituted with F, or C₁₋₄ heteroalkyl, such as C₁₋₄ alkoxy, optionally substituted with F.

In some embodiments according to Formula A (e.g., Formula A1a, A1b, A1c, A1d, A1e, A1f, A1g, or A1h), Y³, Y⁴, and Y⁵ together represent an optionally substituted 5 or 6-membered heteroarylene, such as a 2,5-thiophenylene

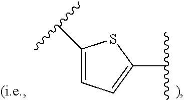 [SMILES: *c1ccc(*)s1]

2,5-furanylene, 2,4-pyridnylene, 2,6-pyridnylene, 3,5-pyridinylene, 2,4-pyrimidinylene, 2,6-pyrimidinylene, or 4,6-pyrimidinylene. In some preferred embodiments, Y³, Y⁴, and Y⁵ together represent an optionally substituted 6-membered heteroarylene. In some embodiments, Y³, Y⁴, and Y⁵ together represent an unsubstituted 6-membered heteroarylene, for example, an unsubstituted 2,4-pyridinylene

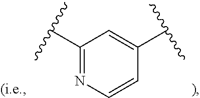 [SMILES: *c1ccnc(*)c1]

2,6-pyridinylene (i.e.

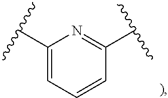 [SMILES: *c1cccc(*)n1]

3,5-pyridinylene

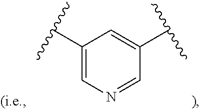 [SMILES: *c1cncc(*)c1]

2,4-pyrimidinylene

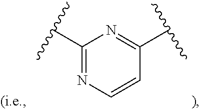 [SMILES: *c1ccnc(*)n1]

2,6-pyrimidinylene

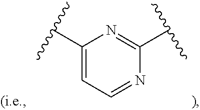 [SMILES: *c1ccnc(*)n1]

or 4,6-pyrimidinylene

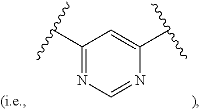 [SMILES: *c1cc(*)ncn1]

In some embodiments, Y³, Y⁴, and Y⁵ together represent a 5 or 6-membered heteroarylene (e.g., described herein), which is substituted with one or two substituents, such as one substituent, each independently selected from halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring). In some embodiments, the optionally substituted groups of G² are unsubstituted. In some embodiments, when substituted, the optionally substituted groups of G² are independently unsubstituted or substituted with one or more (e.g., 1, 2, or 3) substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring. In some preferred embodiments, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

In some embodiments according to Formula A (e.g., Formula A1a, A1b, A1e, A1d, A1e, A1f, A1g, or A1h), Y³, Y⁴, and Y⁵ together can also represent a 3-6 membered carbocyclic or heterocyclic ring, which is optionally substituted with one or more substituents each independently oxo, halogen, OH, NH₂, C₁₋₄ alkyl optionally substituted with F, C₁₋₄ alkoxy optionally substituted with F, or a 3-4 membered carbocyclic or heterocyclic ring optionally substituted with F and/or methyl. For example, in some embodiments, Y³, Y⁴, and Y⁵ can together represent

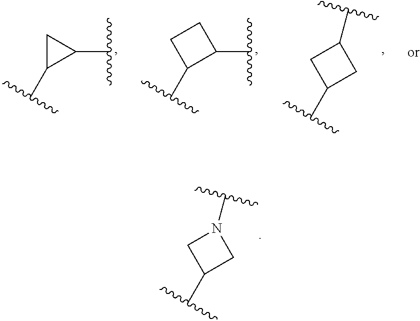 [SMILES: *C1CC1* ; *C1CCC1* ; *C1CC(*)C1 ; *C1CN(*)C1]

In some embodiments according to Formula A (e.g., A1), Y², Y³, and Y⁴ together represent an optionally substituted 3-8 membered ring, and Y¹, Y⁵, Y⁶, and Y⁷ are as defined herein, e.g., in enumerated Embodiment 1-(1). In some embodiments, Y¹, Y⁵, Y⁶, and Y⁷ can be any of those defined in enumerated Embodiments 5, 14, and 16-18, respectively.

In some embodiments according to Formula A (e.g., A1), Y², Y³, Y⁴, and Y⁵ together represent an optionally substituted 3-10 membered ring, and Y¹, Y⁶, and Y⁷ are as defined herein, e.g., in enumerated Embodiment 1-(1). In some embodiments, Y¹, Y⁶, and Y⁷ can be any of those defined in enumerated Embodiments 5 and 16-18, respectively.

In some embodiments according to Formula A (e.g., A1), Y², Y³, Y⁴, Y⁵, and Y⁶ together represent an optionally substituted 3-10 membered ring, and Y¹ and Y⁷ are as defined herein, e.g., in enumerated Embodiment 1-(1). In some embodiments, Y¹ and Y⁷ can be any of those defined in enumerated Embodiments 5 and 17, respectively.

In some embodiments according to Formula A (e.g., A1), Y⁴, Y⁵, and Y⁶ together represent an optionally substituted 3-8 membered ring, and Y¹, Y², Y³, and Y⁷ are as defined herein, e.g., in enumerated Embodiment 1-(1). In some embodiments, Y¹, Y², Y³ and Y⁷ can be any of those defined in enumerated Embodiments 5, 6, 12, and 17, respectively. In some preferred embodiments, Y¹-Y² can be a NHCH₂ or OCH₂ linker, and Y⁷ can be —C(O)—, —N(CH₃)—, —CH₂—, or —O—. In some embodiments, Y⁴, Y⁵, and Y⁶ together represent an optionally substituted 3-6 membered carbocyclic or heterocyclic ring, optionally substituted phenylene, or optionally substituted 5 or 6-membered heteroarylene.

In some embodiments according to Formula A (e.g., A1), Y⁵, Y⁶, and Y⁷ together represent an optionally substituted 3-8 membered ring, and Y¹, Y², Y³, and Y⁴ are as defined herein, e.g., in enumerated Embodiment 1-(1). In some embodiments, Y¹, Y², Y³, and Y⁴ can be any of those defined in enumerated Embodiments 5, 6, 12, and 13, respectively. In some embodiments, Y¹-Y² can be a NHCH₂ or OCH₂ linker, and Y³-Y⁴ can be —C(O)NH—, —C(O)—N(CH₃)—, —NHCH₂—, or —OCH₂—. In some embodiments, Y⁵, Y⁶, and Y⁷ together represent an optionally substituted 3-8 membered carbocyclic ring, 3-8 membered heterocyclic ring, phenyl ring, or heteroaryl ring. For example, in some embodiments, Y⁵, Y⁶, and Y⁷ together represent

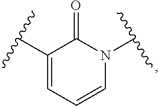 [SMILES: *c1cccn(*)c1=O]

preferably, the nitrogen of the pyridin-2-one is attached to Y⁸.

In some embodiments according to Formula A (e.g., A1), Y³, Y⁴, Y⁵, and Y⁶ together represent an optionally substituted 3-10 membered ring, and Y¹, Y², and Y⁷ are as defined herein, e.g., in enumerated Embodiment 1-(1). In some embodiments, Y¹, Y², and Y³ can be any of those defined in enumerated Embodiments 5, 6, and 17, respectively.

In some embodiments according to Formula A (e.g., A1), Y⁴, Y⁵, Y⁶, and Y⁷ together represent an optionally substituted 3-10 membered ring, and Y¹, Y², and Y³ are as defined herein, e.g., in enumerated Embodiment 1-(1). In some embodiments, Y¹, Y², and Y³ can be any of those defined in enumerated Embodiments 5, 6, and 12, respectively. In some embodiments, Y¹-Y² can be a NHCH₂ or OCH₂ linker, and Y³ can be —CH₂—. In some embodiments, Y⁴, Y⁵, Y⁶, and Y⁷ together represent an optionally substituted 3-8 membered carbocyclic ring, 3-8 membered heterocyclic ring, phenyl ring, or heteroaryl ring. For example, in some embodiments, Y⁴, Y⁵, Y⁶, and Y⁷ together represent

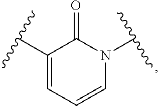 [SMILES: *c1cccn(*)c1=O]

preferably, the nitrogen of the pyridin-2-one is attached to Y⁸.

In some embodiments according to Formula A (e.g., A1), Y², Y³, Y⁴, Y⁵, Y⁶, and Y⁷ together represent an optionally substituted 8-10 membered heterocyclic or heteroaryl ring, and Y¹ is as defined herein, e.g., in enumerated Embodiment 1-(1). In some embodiments, Y¹ is defined in enumerated Embodiment 5. In some embodiments, Y¹ is NH or O.

In some embodiments according to Formula A (e.g., A1), Y³, Y⁴, Y⁵, Y⁶, and Y⁷ together represent an optionally substituted 8-10 membered heterocyclic or heteroaryl ring, preferably, a 6,5-fused or 6,6-fused heterocyclic or heteroaryl ring, having 1-5 ring heteroatoms each independently O, N, or S, and Y¹ and Y² are as defined herein, e.g., in enumerated Embodiment 1-(1). In some embodiments, Y¹ and Y² can be any of those defined in enumerated Embodiments 5 and 6, respectively. In some embodiments, Y¹-Y² can be a NHCH₂ or OCH₂ linker. In some embodiments, Y³, Y⁴, Y⁵, Y⁶, and Y⁷ together represent an optionally substituted 6,5-fused or 6,6-fused heterocyclic ring, in which a phenyl ring or 6-membered heteroaryl is fused with a 5- or 6-membered heterocyclic ring having 1 or 2 ring heteroatoms each independently O, N, or S, preferably, 1 ring nitrogen atom, for example, Y³, Y⁴, Y⁵, Y⁶, and Y⁷ together represent

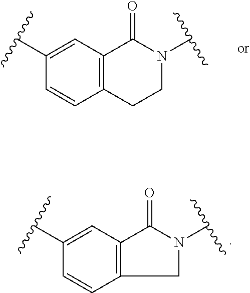 [SMILES: *c1ccc2c(c1)C(=O)N(*)CC2 ; *c1ccc2c(c1)C(=O)N(*)C2]

For example, in some embodiments, the compound of Formula A can have a structure according to Formula A1j or A1k:

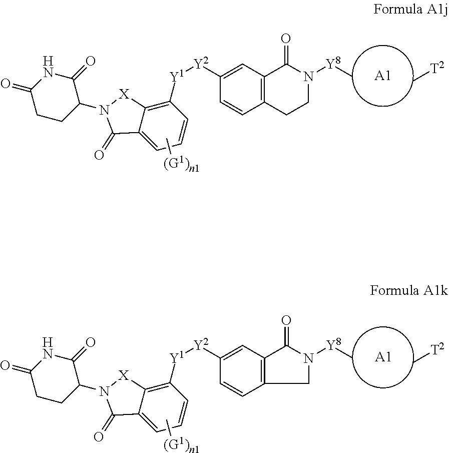

wherein the variables are defined herein.

In some embodiments, Y³, Y⁴, Y⁵, Y⁶, and Y⁷ together can also represent an optionally substituted 6,5-fused or 6,6-fused heteroaryl ring having 1-5 ring heteroatoms each independently O, N, or S. For example, in some embodiments, Y³, Y⁴, Y⁵, Y⁶, and Y⁷ together can be an optionally substituted benzimidazole, indazole, benzothiophene, etc. In some embodiments, the benzimidazole, indazole, or benzothiophene is unsubstituted. In some embodiments, when substituted, the 6,5-fused or 6,6-fused heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

In some embodiments according to Formula A (e.g., A1), Y¹-Y⁷ together can also be any of the specific linkers shown in specific compounds described herein, e.g., in Examples 1-150.

Typically, in Formula A (e.g., A1, such as A1a to A1k), Y⁸ is null. However, in some embodiments, Y⁸ can also be O, NH, C(O), C₁₋₂ alkylene, or C₁₋₆ heteroalkylene having 1-3 heteroatoms independently selected from O, N, and S, wherein the S is optionally oxidized, and the C₁₋₆ heteroalkylene is optionally substituted with 1 or 2 oxo groups.

In Formula A (e.g., A1, such as A1a to A1k), Ring A1 is preferably a 4-8 membered monocyclic carbocyclic or heterocyclic ring optionally substituted with one or more (e.g., 1 or 2) substituents each independently halogen, OH, CN, oxo, C₁₋₄ alkyl optionally substituted with F, or C₁₋₄ heteroalkyl optionally substituted with F.

In some preferred embodiments according to Formula A (e.g., A1, such as A1a to A1k), Ring A1 is a C₄₋₇cycloalkylene optionally substituted with one or more substituents each independently halogen, OH, CN, oxo, C₁₋₄ alkyl optionally substituted with F, or C₁₋₄ heteroalkyl optionally substituted with F. In some embodiments, Ring A1 is an unsubstituted C₄₋₇ cycloalkylene. For example, in some preferred embodiments, Ring A1 is cyclohexylene, preferably, a 1,4-trans-cyclohexylene,

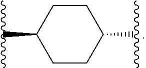 [SMILES: *[C@H]1CC[C@H](*)CC1]

T² in Formula A (including any of the subformulae) is preferably not hydrogen. As described herein, the inventors have found that it is beneficial to include a fused bicyclic or tricyclic heteroaryl as T² for compounds of Formula A, which can afford compounds with a good potency, such as a lower DC₅₀ in connection with GSPT1.

In Formula A (e.g., A1, such as A1a to A1k), T² is preferably an optionally substituted 5,5-fused or 6,5-fused heteroaryl ring having 1-5 ring heteroatoms each independently O, N, or S. More preferably, T² connects to Ring A1 through a ring atom on the 5-membered ring. In some embodiments, T² is an optionally substituted thiazolopyridine, imidazolopyridine, benzimidazole, pyrazolopyridine, oxazolopyridine, benzoxazole, indole, benzothiophene, benzothiazole, thienopyridine, thienopyrimidine, or thienothiophene, etc. Preferably, when substituted, the 5,5-fused or 6,5-fused heteroaryl ring herein is substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1} C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

In some preferred embodiments, in Formula A (e.g., A1, such as A1a to A1k), T² is an optionally substituted 6,5-fused heteroaryl ring having 1-5 ring heteroatoms each independently O, N, or S, characterized in that one of the fused rings is a furan, thiophene, pyrrole, pyrazole, imidazole, oxazole, thiazole, isoxazole, or isothiazole, which is fused with a benzene, pyridine, pyrimidine, pyridazine, or pyrazine. Preferably, T² connects to Ring A1 through a ring atom on the 5-membered ring. To be clear, in embodiments herein, a bicyclic fused heteroaryl or tricyclic fused heteroaryl may be described by the name of individual rings, for example, when it is stated that in a bicyclic fused heteroaryl, one of the fused rings is an imidazole, which is fused with a pyrazine ring, it should be understood that the bicyclic fused heteroaryl includes

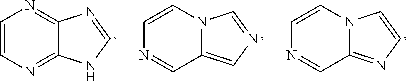

etc., i.e., the bicyclic fused heteroaryl includes all possible imidazopyrazine, including imidazo[1,2-a]pyrazine, imidazo[1,5-a]pyrazine, or imidazo[4,5-b]pyrazine. Similar expressions regarding other fused bicyclic or tricyclic heteroaryl should be understood similarly. In some preferred embodiments, the 6,5-fused heteroaryl is a thiazole fused to a benzene, pyridine, pyrimidine, pyridazine, or pyrazine, more preferably, a thiazole fused to a pyridine, which can be

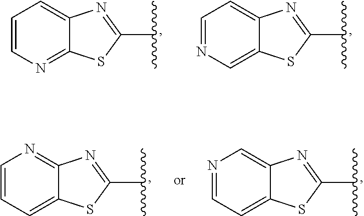 [SMILES: *c1nc2cccnc2s1 ; *c1nc2ccncc2s1 ; *c1nc2ncccc2s1 ; *c1nc2cnccc2s1]

and optionally substituted with one or more suitable substituents herein. In some preferred embodiments, the 6,5-fused heteroaryl is an imidazole fused to a benzene, pyridine, pyrimidine, pyridazine, or pyrazine, which can

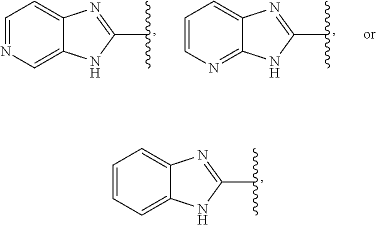

and optionally substituted with one or more suitable substituents herein. In some embodiments, the 6,5-fused heteroaryl ring is unsubstituted. In some embodiments, the 6,5-fused heteroaryl ring is substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl. In some embodiments, the 6,5-fused heteroaryl ring is unsubstituted or substituted with 1 or 2 substituents each independently halogen, CN, OH, NH₂, a C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} and G^{S2} are defined herein. In some embodiments, the 6,5-fused heteroaryl ring is unsubstituted or substituted with one substituent selected from a C₁₋₄ alkyl optionally substituted with 1-3 F or C₁₋₄ heteroalkyl optionally substituted with 1-3 F, such as methoxy. In some embodiments, the 6,5-fused heteroaryl ring is unsubstituted or substituted with one or two substituents each independently selected from halogen, halogen, CN, OH, NH₂, a C₁₋₄ alkyl optionally substituted with 1-3 F or C₁₋₄ heteroalkyl optionally substituted with 1-3 F, such as methoxy.

In some more specific embodiments, in Formula A (e.g., A1, such as A1a to A1k), T² is an optionally substituted 6,5-fused heteroaryl selected from:

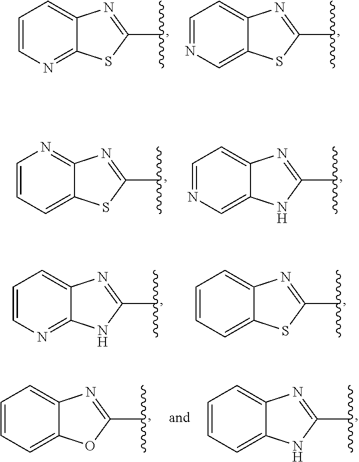

each of which is optionally substituted with 1-3 substituents each independently halogen, CN, G², OH, NH₂, O-G², NHG², NG²G², COOH, CONH₂, C(O)O-G², C(O)NHG², or C(O)NG²G², wherein G² is defined herein. To be clear, the substituents, when present, can attach to a ring carbon or ring nitrogen atom when applicable. In some embodiments, the foregoing 6,5-fused heteroaryl is unsubstituted or substituted with 1 or 2 substituents each independently a C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} and G^{S2} are defined herein. In some embodiments, the 6,5-fused heteroaryl ring is unsubstituted or substituted with one substituent selected from a C₁₋₄ alkyl optionally substituted with 1-3 F or C₁₋₄ heteroalkyl optionally substituted with 1-3 F, such as methoxy. In some embodiments, the 6,5-fused heteroaryl ring is unsubstituted or substituted with one or two substituents each independently selected from halogen, CN, OH, NH₂, a C₁₋₄ alkyl optionally substituted with 1-3 F or C₁₋₄ heteroalkyl optionally substituted with 1-3 F, such as methoxy. In some more specific embodiments, in Formula A (e.g., A1, such as A1a to A1k), T² can be a 6,5-fused heteroaryl as shown in any of the specific compounds of Examples 1-150.

In some embodiments, in Formula A (e.g., A1, such as A1a to A1k), T² can also be an optionally substituted 5,6,5-fused, 6,5,5-fused, or 5,6,6-fused tricyclic heteroaryl ring having 1-5 ring heteroatoms each independently O, N, or S, wherein each 5-membered ring of the fused rings is independently a furan, thiophene, pyrrole, pyrazole, imidazole, oxazole, thiazole, isoxazole, or isothiazole, and each 6-membered ring of the fused rings is independently benzene, pyridine, pyrimidine, pyridazine, or pyrazine. In some embodiments, one 5-membered ring is an imidazole. In some embodiments, one 5-membered ring is a pyrrole. In some embodiments, one 6-membered ring is a pyridine. In some embodiments, the 5,6,5-fused, 6,5,5-fused, or 5,6,6-fused tricyclic heteroaryl ring is unsubstituted. In some embodiments, the 5,6,5-fused, 6,5,5-fused, or 5,6,6-fused tricyclic heteroaryl ring is substituted with one or more substituents defined herein. In some embodiments, the 5,6,5-fused, 6,5,5-fused, or 5,6,6-fused tricyclic heteroaryl ring is substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl. In some embodiments, the 5,6,5-fused, 6,5,5-fused, or 5,6,6-fused tricyclic heteroaryl ring is unsubstituted or substituted with 1 or 2 substituents each independently halogen, CN, OH, NH₂, a C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} and G^{S2} are defined herein. In some embodiments, T² is a heteroaryl selected from:

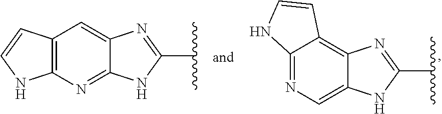 [SMILES: *c1nc2cc3cc[nH]c3nc2[nH]1 ; *c1nc2c(cnc3[nH]ccc32)[nH]1]

which is optionally substituted with one or two substituents independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} and G^{S2} are defined herein In some more specific embodiments, in Formula A (e.g., A1, such as A1a to A1k), T² can be a 5,6,5-fused heteroaryl as shown in any of the specific compounds of Examples 1-150.

### Formula B

In some embodiments, the present disclosure also provides a compound according to Formula B, as defined in any of the enumerated Embodiments 32-39, or a pharmaceutically acceptable salt thereof.

In Formula B, LNK is not particularly limited, which can be a chain, ring, or a ring-chain structure. In some embodiments, LNK is a linker of a chain structure, which means that the linker can connect T¹ with the cyclohexylene ring without using any ring forming atoms (or ring atoms for short). To be clear, such linker may optionally have one or more rings as substituents. In some embodiments, LNK is a linker of a ring-chain structure, which means that the linker connects T¹ with the cyclohexylene ring using at least one chain atom and at least one ring atom. In some embodiments, LNK is a linker of a ring structure, which means that the linker connects T¹ with the cyclohexylene ring without using a chain atom. To be clear, such ring structure linker may optionally have one or more substituents having one or more chain atoms. For example,

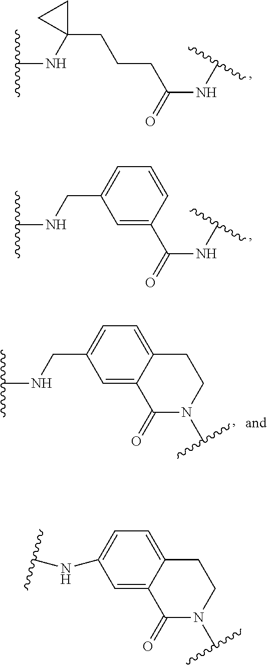

are all considered a ring-chain structure for the linker herein, whereas

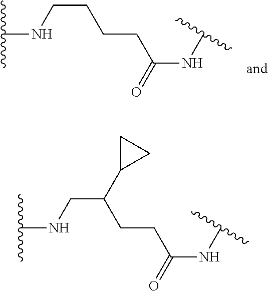 [SMILES: *NCCCCC(=O)N* ; *NCC(CCC(=O)N*)C1CC1]

would be considered as chain structure for the linker herein, and

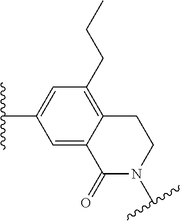 [SMILES: *c1cc(CCC)c2c(c1)C(=O)N(*)CC2]

is a ring structure for the linker herein. Typically, LNK is characterized as having certain “length”, reflected by the smallest number of chain or ring forming atoms of the linker, which is at least 4 (e.g., 4, 5, 6, 7, 8, or 9), wherein each of the chain or ring forming atoms is independently selected from C, N, O, and S, wherein the smallest number is the least number of atoms of the linker needed to reach from T¹ to the cyclohexyl ring, starting from the atom that is bonded to T¹ and ending with the atom that is bonded to the cyclohexyl ring. To explain, when LNK is a linker of

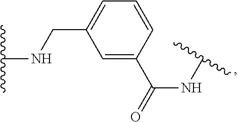 [SMILES: *NCc1cccc(C(=O)N*)c1]

the smallest number of chain or ring forming atoms of the linker is 7, which is counted from the bolded N atom bonded to T¹, including the least number of atoms, i.e., the carbon atoms which are the chain or ring forming atoms for the bolded bonds below, to reach the bolded N atom that is bonded to the cyclohexylene:

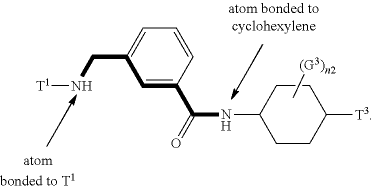

It should be apparent that the alternate path through the other three ring atoms of the phenyl ring (non-bolded bonds) would increase the atom count to 9 and thus not the least number of atoms of the linker needed to reach from T¹ to the cyclohexyl ring. Based on such, it is also clear that any substituents on the phenyl ring or the N atom or the methylene group in the linker above would not be counted towards the smallest number. Linkers of other structures should be understood similarly. In some embodiments, the smallest number of chain or ring forming atoms of the linker is 4-10, typically, the smallest number of chain or ring forming atoms of the linker has at most 6 of which from ring forming atoms. In some embodiments, the smallest number of chain or ring forming atoms of the linker can also be greater than 10, such as 12, 15, or 20, or any range in between. Typically, the molecular weight for the LNK ranges from about 50-350 Da, preferably, 50-250, Da such as about 80-180 Da or about 100-200 Da. Typically, LNK has no more than 8 heteroatoms. For example, in some embodiments, LNK has 2-6 (e.g., 2, 3, 4, or 5) heteroatoms, each independently O, N, or S, with the remaining atoms C, H, D, or halogen. Typically, the LNK has at most 3 rings, for example, 1 or 2 monocyclic rings, or 1 bicyclic ring, or 1 monocyclic ring and 1 bicyclic ring. In some embodiments, LNK has one 5 or 6 membered ring which is carbocylic, heterocyclic, phenyl, or heteroaryl. In some embodiments, LNK has a bicyclic heterocylic or heteroaryl ring. In some embodiments, LNK has a spiro 3-6 membered ring, such as a spiro cyclopropyl ring.

In some embodiments according to Formula B (including any of its subformulae), LNK can have any of the definitions of

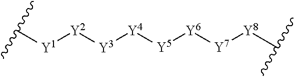

as defined herein, including any of those defined in enumerated Embodiments 5-25.

In some embodiments according to Formula B (including any of its subformulae), LNK can have any of the definitions of L as defined in connection with Formula (I).

In some embodiments according to Formula B (including any of its subformulae), LNK can have any of the linker definitions as shown in the specific structures herein, such as those shown in Examples 1-150.

Typically, in Formula B, n2 is 0, and the compound can have a structure according to Formula B1, in which the cyclohexylene is a 1,4-trans cyclohexylene.

In some embodiments, the compound of Formula B can have one or two G³ substituents on the cyclohexylene (preferably, 1,4-trans cyclohexylene). Typically, when substituted the G³ substituent is not attached to the carbon atoms that are bonded to LNK or T³. When present, G³ is typically F, OH, CN, C₁₋₂ alkyl optionally substituted with F, or C₁₋₃ heteroalkyl optionally substituted with F.

Typically, in Formula B (e.g., Formula B1 or B2), T³ is an optionally substituted 5,5-fused or 6,5-fused heteroaryl ring having 1-5 ring heteroatoms each independently O, N, or S. More preferably, T³ connects to the cyclohexylene through a ring atom on the 5-membered ring. In some embodiments, T³ is an optionally substituted thiazolopyridine, imidazolopyridine, benzimidazole, pyrazolopyridine, oxazolopyridine, benzoxazole, indole, benzothiophene, benzothiazole, thienopyridine, thienopyrimidine, or thienothiophene, etc. Preferably, when substituted, the 5,5-fused or 6,5-fused heteroaryl ring herein is substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

In some preferred embodiments, in Formula B (e.g., Formula B1 or B2), T³ is an optionally substituted 6,5-fused heteroaryl ring having 1-5 ring heteroatoms each independently O, N, or S, characterized in that one of the fused rings is a furan, thiophene, pyrrole, pyrazole, imidazole, oxazole, thiazole, isoxazole, or isothiazole, which is fused with a benzene, pyridine, pyrimidine, pyridazine, or pyrazine. Preferably, T³ connects to the cyclohexylene through a ring atom on the 5-membered ring. In some preferred embodiments, the 6,5-fused heteroaryl is a thiazole fused to a benzene, pyridine, pyrimidine, pyridazine, or pyrazine, more preferably, a thiazole fused to a pyridine, which can be

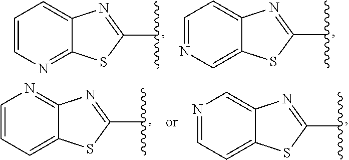

and optionally substituted with one or more suitable substituents herein. In some preferred embodiments, the 6,5-fused heteroaryl is an imidazole fused to a benzene, pyridine, pyrimidine, pyridazine, or pyrazine, which can be

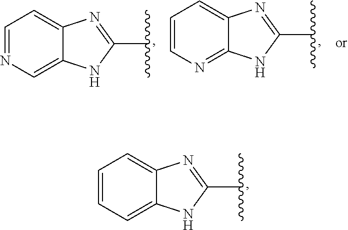

and optionally substituted with one or more suitable substituents herein. In some embodiments, the 6,5-fused heteroaryl ring is unsubstituted. In some embodiments, the 6,5-fused heteroaryl ring is substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂-4 alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl. In some embodiments, the 6,5-fused heteroaryl ring is unsubstituted or substituted with 1 or 2 substituents each independently a C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} and G^{S2} are defined herein. In some embodiments, the 6,5-fused heteroaryl ring is unsubstituted or substituted with one substituent selected from a C₁₋₄ alkyl optionally substituted with 1-3 F or C₁₋₄ heteroalkyl optionally substituted with 1-3 F, such as methoxy. In some embodiments, the 6,5-fused heteroaryl ring is unsubstituted or substituted with one or two substituents each independently selected from halogen, CN, OH, NH₂, a C₁₋₄ alkyl optionally substituted with 1-3 F or C₁₋₄ heteroalkyl optionally substituted with 1-3 F, such as methoxy.

In some more specific embodiments, in Formula B (e.g., Formula B1 or B2), T³ is an optionally substituted 6,5-fused heteroaryl selected from:

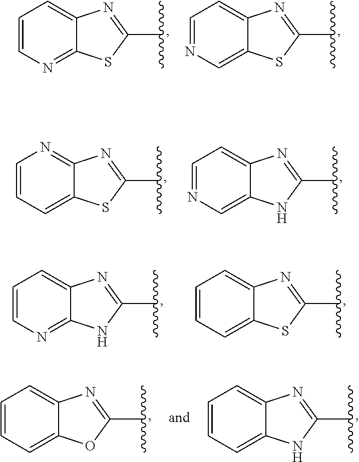

each of which is optionally substituted with 1-3 substituents each independently halogen, CN, G², OH, NH₂, O-G², NHG², NG²G², COOH, CONH₂, C(O)O-G², C(O)NHG², or C(O)NG²G², wherein G² is defined herein. To be clear, the substituents, when present, can attach to a ring carbon or ring nitrogen atom when applicable. In some embodiments, the foregoing 6,5-fused heteroaryl is unsubstituted or substituted with 1 or 2 substituents each independently halogen, CN, OH, NH₂, a C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} and G^{S2} are defined herein. In some embodiments, the 6,5-fused heteroaryl ring is unsubstituted or substituted with one substituent selected from a C₁₋₄ alkyl optionally substituted with 1-3 F or C₁₋₄ heteroalkyl optionally substituted with 1-3 F, such as methoxy. In some embodiments, the 6,5-fused heteroaryl ring is unsubstituted or substituted with or two substituents each independently selected from halogen, CN, OH, NH₂, a C₁₋₄ alkyl optionally substituted with 1-3 F or C₁₋₄ heteroalkyl optionally substituted with 1-3 F, such as methoxy. In some more specific embodiments, in Formula B (e.g., Formula B1 or B2), T³ can be a 6,5-fused heteroaryl as shown in any of the specific compounds of Examples 1-150.

In some embodiments, in Formula B (e.g., Formula B1 or B2), T³ can also be an optionally substituted 5,6,5-fused, 6,5,5-fused, or 5,6,6-fused tricyclic heteroaryl ring having 1-5 ring heteroatoms each independently O, N, or S, wherein each 5-membered ring of the fused rings is independently a furan, thiophene, pyrrole, pyrazole, imidazole, oxazole, thiazole, isoxazole, or isothiazole, and each 6-membered ring of the fused rings is independently benzene, pyridine, pyrimidine, pyridazine, or pyrazine. In some embodiments, one 5-membered ring is an imidazole. In some embodiments, one 5-membered ring is a pyrrole. In some embodiments, one 6-membered ring is a pyridine. In some embodiments, the 5,6,5-fused, 6,5,5-fused, or 5,6,6-fused tricyclic heteroaryl ring is unsubstituted. In some embodiments, the 5,6,5-fused, 6,5,5-fused, or 5,6,6-fused tricyclic heteroaryl ring is substituted with one or more substituents defined herein. In some embodiments, the 5,6,5-fused, 6,5,5-fused, or 5,6,6-fused tricyclic heteroaryl ring is substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl. In some embodiments, the 5,6,5-fused, 6,5,5-fused, or 5,6,6-fused tricyclic heteroaryl ring is unsubstituted or substituted with 1 or 2 substituents each independently halogen, CN, OH, NH₂, a C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} and G^{S2} are defined herein. In some embodiments, T³ is a heteroaryl selected from:

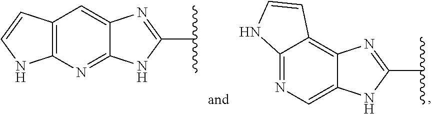 [SMILES: *c1nc2cc3cc[nH]c3nc2[nH]1 ; *c1nc2c(cnc3[nH]ccc32)[nH]1]

which is optionally substituted with one or two substituents independently halogen, CN, OH, NH₂, C₁₋₄ alkyl optionally substituted with 1-3 G^{S1} C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} and G^{S2} are defined herein In some more specific embodiments, in Formula B (e.g., Formula B1 or B2), T³ can be a 5,6,5-fused heteroaryl as shown in any of the specific compounds of Examples 1-150.

T¹ for Formula B (e.g., Formula B1 or B2) can be any of those described herein in connection with Formula (I) or A. In some embodiments, T¹ for Formula B (e.g., Formula B1 or B2) is as defined in any of enumerated Embodiments 36-38. In some embodiments, T¹ for Formula B (e.g., Formula B1 or B2) can be any of the glutarimide containing moiety as shown in any of the specific compounds of Examples 1-150.

### Formula C

In some embodiments, the present disclosure also provides a compound according to Formula C, as defined in any of the enumerated Embodiments 40-49, or a pharmaceutically acceptable salt thereof.

In some embodiments according to Formula C, T⁴ is an optionally substituted 5,5-fused or 6,5-fused heteroaryl ring having 1-5 ring heteroatoms each independently O, N, or S, in which a ring atom of a 5-membered ring of T⁴ is bonded to Ring A1. In some embodiments according to Formula C, T⁴ is an optionally substituted thiazolopyridine, imidazolopyridine, benzimidazole, pyrazolopyridine, oxazolopyridine, benzoxazole, indole, benzothiophene, benzothiazole, thienopyridine, thienopyrimidine, or thienothiophene, etc. Preferably, when substituted, the 5,5-fused or 6,5-fused heteroaryl ring herein is substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{Sl}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

In some preferred embodiments, in Formula C (e.g., Formula C₁), T⁴ is an optionally substituted 6,5-fused heteroaryl ring having 1-5 ring heteroatoms each independently O, N, or S, characterized in that one of the fused rings is a furan, thiophene, pyrrole, pyrazole, imidazole, oxazole, thiazole, isoxazole, or isothiazole, which is fused with a benzene, pyridine, pyrimidine, pyridazine, or pyrazine. Preferably, T⁴ connects to the Ring A1 through a ring atom on the 5-membered ring. In some preferred embodiments, the 6,5-fused heteroaryl is a thiazole fused to a benzene, pyridine, pyrimidine, pyridazine, or pyrazine, more preferably, a thiazole fused to a pyridine, which can be

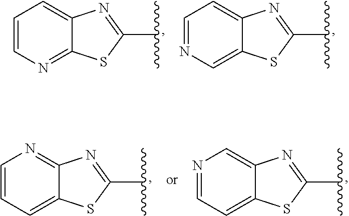

optionally substituted with one or more suitable substituents herein. In some preferred embodiments, the 6,5-fused heteroaryl is an imidazole fused to a benzene, pyridine, pyrimidine, pyridazine, or pyrazine, which can be

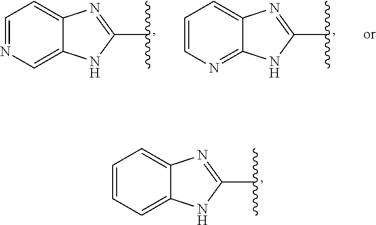

and optionally substituted with one or more suitable substituents herein. In some embodiments, the 6,5-fused heteroaryl ring is unsubstituted. In some embodiments, the 6,5-fused heteroaryl ring is substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl. In some embodiments, the 6,5-fused heteroaryl ring is unsubstituted or substituted with 1 or 2 substituents each independently halogen, CN, OH, NH₂, a C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} and G^{S2} are defined herein. In some embodiments, the 6,5-fused heteroaryl ring is unsubstituted or substituted with one substituent selected from a C₁₋₄ alkyl optionally substituted with 1-3 F or C₁₋₄ heteroalkyl optionally substituted with 1-3 F, such as methoxy. In some embodiments, the 6,5-fused heteroaryl ring is unsubstituted or substituted with one or two substituents each independently selected from halogen, CN, OH, NH₂, a C₁₋₄ alkyl optionally substituted with 1-3 F or C₁₋₄ heteroalkyl optionally substituted with 1-3 F, such as methoxy.

In some more specific embodiments, in Formula C (e.g., Formula C₁), T⁴ is an optionally substituted 6,5-fused heteroaryl selected from:

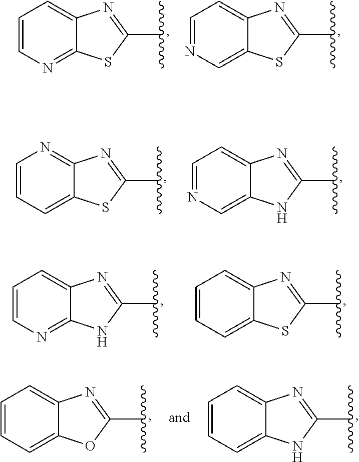

each of which is optionally substituted with 1-3 substituents each independently halogen, CN, G², OH, NH₂, O-G², NHG², NG²G², COOH, CONH₂, C(O)O-G², C(O)NHG², or C(O)NG²G², wherein G² is defined herein. To be clear, the substituents, when present, can attach to a ring carbon or ring nitrogen atom when applicable. In some embodiments, the foregoing 6,5-fused heteroaryl is unsubstituted or substituted with 1 or 2 substituents each independently halogen, CN, OH, NH₂, a C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} and G^{S2} are defined herein. In some embodiments, the 6,5-fused heteroaryl ring is unsubstituted or substituted with one substituent selected from a C₁₋₄ alkyl optionally substituted with 1-3 F or C₁₋₄ heteroalkyl optionally substituted with 1-3 F, such as methoxy. In some embodiments, the 6,5-fused heteroaryl ring is unsubstituted or substituted with one or two substituents each independently selected from halogen, CN, OH, NH₂, a C₁₋₄ alkyl optionally substituted with 1-3 F or C₁₋₄ heteroalkyl optionally substituted with 1-3 F, such as methoxy. In some more specific embodiments, in Formula C (e.g., Formula C₁), T⁴ can be a 6,5-fused heteroaryl as shown in any of the specific compounds of Examples 1-150.

In some embodiments according to Formula C, T⁴ is an optionally substituted 5,6,5-fused, 6,5,5-fused, or 5,6,6-fused tricyclic heteroaryl ring having 1-6 ring heteroatoms each independently O, N, or S, in which a ring atom of a 5-membered ring of T⁴ is bonded to Ring A1. In some embodiments, each 5-membered ring of the fused rings is independently a furan, thiophene, pyrrole, pyrazole, imidazole, oxazole, thiazole, isoxazole, or isothiazole, and each 6-membered ring of the fused rings is independently benzene, pyridine, pyrimidine, pyridazine, or pyrazine. In some embodiments, one 5-membered ring is an imidazole. In some embodiments, one 5-membered ring is a pyrrole. In some embodiments, one 6-membered ring is a pyridine. In some embodiments, the 5,6,5-fused, 6,5,5-fused, or 5,6,6-fused tricyclic heteroaryl ring is unsubstituted. In some embodiments, the 5,6,5-fused, 6,5,5-fused, or 5,6,6-fused tricyclic heteroaryl ring is substituted with one or more substituents defined herein. In some embodiments, the 5,6,5-fused, 6,5,5-fused, or 5,6,6-fused tricyclic heteroaryl ring is substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl. In some embodiments, the 5,6,5-fused, 6,5,5-fused, or 5,6,6-fused tricyclic heteroaryl ring is unsubstituted or substituted with 1 or 2 substituents each independently halogen, CN, OH, NH₂, a C₁₋₄ alkyl optionally substituted with 1-3 G^{S1}, C₁₋₄ heteroalkyl optionally substituted with 1-3 G^{S2} or a 3-7 membered ring (preferably 3-7 membered carbocyclic or heterocyclic ring) optionally substituted with 1-3 G^{S2}, wherein G^{S1} and G^{S2} are defined herein. In some embodiments, T³ is a heteroaryl selected from:

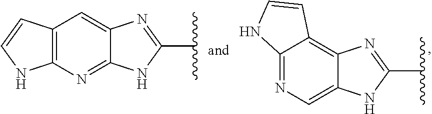 [SMILES: *c1nc2cc3cc[nH]c3nc2[nH]1 ; *c1nc2c(cnc3[nH]ccc32)[nH]1]

which is optionally substituted with one or two substituents independently halogen, CN, OH, NH₂, C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} and G^{S2} are defined herein In some more specific embodiments, in Formula C (e.g., Formula C₁), T⁴ can be a 5,6,5-fused heteroaryl as shown in any of the specific compounds of Examples 1-150.

T¹ and LNK for Formula C (e.g., Formula C₁) can be any of those described herein in connection with Formula B. For example, in some embodiments, LNK for Formula C can be any of those linkers according to

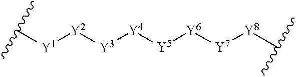

as defined in any of enumerated Embodiments 5-25, or any linker as defined herein for L in connection with Formula (I).

Ring A1 for Formula C (e.g., Formula C₁) can be any of those described herein in connection with Formula A. For example, typically, Ring A1 can be optionally substituted 4-8 membered monocyclic carbocyclic or heterocyclic ring. In some preferred embodiments, Ring A1 can be an optionally substituted C₄₋₇cycloalkylene, such as unsubstituted C₄₋₇cycloalkylene, preferably, cyclohexylene, e.g., 1,4-trans-cyclohexylene,

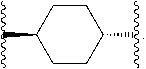 [SMILES: *[C@H]1CC[C@H](*)CC1]

In some embodiments, the present disclosure provides a pharmaceutical composition comprising one or more compounds of the present disclosure (e.g., any of the compounds according to Formula (I), Formula A, Formula B, Formula C, or a subformula thereof, or any of compounds shown in the tables herein, or any of Examples 1-150, a deaerated analog thereof, a stereoisomer thereof, a solvate or hydrate thereof, or a pharmaceutically acceptable salt thereof). In some embodiments, the pharmaceutical composition comprises one or more compounds of the present disclosure that has a DC₅₀ for GSPT1 of less than 100 nM as measured according to the methods described herein. The pharmaceutical compositions herein can be formulated for any suitable route of administration, such as oral or parenteral administration.

In some embodiments, the present disclosure provides a method of inducing degradation of a protein (in particular, GSPT1) in a cell, the method comprising contacting the cell with one or more compounds of the present disclosure (e.g., any of the compounds according to Formula (I), Formula A, Formula B, Formula C, or a subformula thereof, or any of compounds shown in the tables herein, or any of Examples 1-150, a deaerated analog thereof, a stereoisomer thereof, a solvate or hydrate thereof, or a pharmaceutically acceptable salt thereof). In some embodiments, the cell is a cancer cell.

In some embodiments, the present disclosure provides a method of treating a GSPT1 associated disease or disorder in a subject in need thereof, the method comprising administering to the subject a therapeutically effective amount of one or more compounds of the present disclosure (e.g., any of the compounds according to Formula (I), Formula A, Formula B, Formula C, or a subformula thereof, or any of compounds shown in the tables herein, or any of Examples 1-150, a deaerated analog thereof, a stereoisomer thereof, a solvate or hydrate thereof, or a pharmaceutically acceptable salt thereof) or a pharmaceutical composition herein. Suitable GSPT1 associated diseases or disorders include any of those described herein. In some embodiments, the one or more compounds of the present disclosure for the methods herein are selected from those having a DC₅₀ for GSPT1 of less than 100 nM as measured according to the methods described herein.

In some embodiments, the present disclosure provides a method of treating cancer in a subject in need thereof, the method comprising administering to the subject a therapeutically effective amount of one or more compounds of the present disclosure (e.g., any of the compounds according to Formula (I), Formula A, Formula B, Formula C, or a subformula thereof, or any of compounds shown in the tables herein, or any of Examples 1-150, a deaerated analog thereof, a stereoisomer thereof, a solvate or hydrate thereof, or a pharmaceutically acceptable salt thereof) or a pharmaceutical composition herein. In some embodiments, the cancer is associated with GSPT1 activity. The types of cancer to be treated by the methods herein are not particularly limited and can include any of those described herein, including the cancer cell lines that have been tested herein. In some embodiments, the one or more compounds of the present disclosure for the methods herein are selected from those having a DC₅₀ for GSPT1 of less than 100 nM as measured according to the methods described herein.

In the methods of treatment herein, the one or more compounds of the present disclosure can be formulated and administered by any suitable route of administration, such as oral or parenteral administration.

Compounds of the present disclosure can be used as a monotherapy or in a combination therapy. In some embodiments according to the methods described herein, one or more compounds of the present disclosure can be administered as the only active ingredient(s). In some embodiments, the method herein further comprises administering to the subject an additional therapeutic agent, such as those described herein. When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

Dosing regimen including doses for the methods described herein can vary and be adjusted, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Definitions

Unless otherwise defined, all scientific and technical terms used herein have the same meaning as commonly understood by those skilled in the art to which the present invention relates.

In the present invention, the term “alkyl” refers to a hydrocarbon chain radical that is linear or branched and does not contain unsaturated bonds, and the hydrocarbon chain radical is linked to other parts of a molecule by a single bond. Typical alkyl groups contain 1 to 10 (e.g., 1, 2, 3, 4, 5, 6, 7, 8, 9, or 10) carbon atoms, preferably 1 to 6 carbon atoms; examples include methyl, ethyl, n-propyl, isopropyl, n-butyl, isobutyl, tert-butyl, n-pentyl, isopentyl, neopentyl, tert-pentyl, n-hexyl, isohexyl, etc. If alkyl is substituted with cycloalkyl, it is correspondingly “cycloalkylalkyl”, such as cyclopropylmethyl, cyclopropylethyl, cyclobutylmethyl, cyclopentylmethyl, or cyclohexylmethyl. If alkyl is substituted with aryl, it is correspondingly “aralkyl”, such as benzyl, benzhydryl or phenethyl. If alkyl is substituted with heterocyclyl, it is correspondingly “heterocyclylalkyl”. In the present invention, C₀ alkyl refers to H, i.e., C₀₋₁₀ alkyl includes H and C₁₋₁₀ alkyl.

The term “alkylene” refers to a hydrocarbon group (divalent alkyl) formed from an alkane molecule by losing two hydrogen atoms, which may be linear or branched and is linked to other parts of a molecule by a single bond. Typical alkylene groups herein contain 1 to 10 (e.g., 1, 2, 3, 4, 5, 6, 7, 8, 9, or 10) carbon atoms, preferably 1 to 6 carbon atoms; examples include methylene (—CH₂—), ethylene, propylene, butylene, etc. In the present invention, C₀ alkylene refers to a single bond, that is, C₀₋₁₀ alkylene includes a single bond and C₁₋₁₀ alkylene.

The term “cycloalkyl” refers to an alicyclic hydrocarbon, for example, containing 1 to 4 monocyclic and/or fused rings and 3 to 18 carbon atoms, preferably 3 to 10 (e.g., 3, 4, 5, 6, 7, 8, 9, or 10) carbon atoms, e.g., cyclopropyl, cyclobutyl, cyclopentyl, cyclohexyl, or adamantyl. The term “alkoxy” refers to a substituent formed from a hydroxy group by substituting the hydrogen atom with alkyl, e.g., an alkoxy group containing 1 to 10 carbon atoms, e.g., methoxy, ethoxy, propoxy, or butoxy.

The term “alkylamino” refers to a substituent formed from an amino group (—NH₂) by substituting one or two of the hydrogen atoms with alkyl, e.g., an alkylamino group containing 1 to 10 carbon atoms, e.g.,

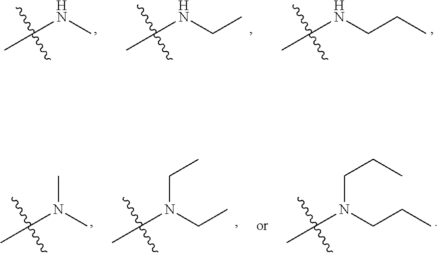

The term “halogen” refers to fluorine, chlorine, bromine, or iodine.

The term “haloalkyl” refers to a group formed from an alkyl group by substituting one or more hydrogen atoms with halogen (e.g., fluorine, chlorine, bromine, or iodine), e.g., —CHF₂, —CH₂F, —CF₃, —CH₂—CF₃, —CH₂CH₂—CF₃, or —CH₂CH₂CH₂—CF₃.

The term “aryl” refers to a monocyclic or polycyclic radical, including polycyclic radicals containing monoaryl and/or fused aryl groups, e.g., containing 1 to 3 monocyclic or fused rings and 6 to 18 (e.g., 6, 8, 10, 12, 14, 16, or 18) carbon ring atoms. The C₆-C₁₂ aryl described herein refers to an aryl group containing 6 to 12 carbon ring atoms, e.g., phenyl, naphthyl, biphenyl, or indenyl.

The term “heterocyclyl” refers to a 3- to 18-membered aromatic or non-aromatic ring group containing 2 to 17 carbon atoms and 1 to 10 heteroatoms. Heterocyclyl may be a monocyclic, bicyclic, tricyclic or tetracyclic ring system, which may include fused, spiro or bridged ring systems. Heterocyclyl may be aromatic (heteroaryl), partially unsaturated, or fully saturated (heterocycloalkyl). Suitable heteroaryl groups for the compound of the present invention contain 1, 2 or 3 heteroatoms selected from N, O and S atoms and include, for example, coumarin, including 8-coumarin, quinolyl, including 8-quinolyl, isoquinolyl, pyridinyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothiophenyl, benzothiazolyl, benzoxazolyl, quinazolinyl, quinoxalinyl, naphthyridinyl, and furopyridinyl. Suitable heterocycloalkyl groups for the compound of the present invention contain 1, 2 or 3 heteroatoms selected from N, O and S atoms and include, for example, pyrrolidinyl, tetrahydrofuryl, dihydrofuran, tetrahydrothienyl, tetrahydrothiopyranyl, piperidinyl, morpholinyl, thiomorpholinyl, oxathianyl, piperazinyl, azetidinyl, oxetanyl, thietanyl, homopiperidinyl, oxiranyl, thiiranyl, azepinyl, oxazepanyl, diazepinyl, triazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolyl, dihydropyranyl, dihydrothienyl, pyrazolidinyl, imidazolinyl, imidazolidinyl, 3-azabicyclo[3.1.0]hexyl, 3-azabicyclo[4.1.0]heptyl, 3H-indolyl, and quinolizinyl.

However, as used in connection with the enumerated embodiments 1-56 herein or related discussions concerning Formula A, B, C, or a subformula thereof, the term “Heterocyclyl” or “heterocyclic” as used by itself or as part of another group refers to a radical of a 3-membered or larger, such as 3- to 14-membered, non-aromatic ring system having ring carbon atoms and at least one ring heteroatom, such as 1 to 4 ring heteroatoms, wherein each heteroatom is independently selected from nitrogen, oxygen, sulfur, boron, phosphorus, and silicon. In other words, in enumerated embodiments 1-56, the term heterocyclyl and heteroaryl are separately defined. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings, and the point of attachment can be on any ring. As used herein, the term “heterocyclylene” as used by itself or as part of another group refers to a divalent radical derived from the heterocyclyl group defined herein. The heterocyclyl or heterocyclylene can be optionally linked to the rest of the molecule through a carbon or nitrogen atom.

In the present invention, “D” represents deuterium; “deuterated” means that one or more hydrogen atoms are substituted with the corresponding number of deuterium atoms. It should be appreciated that there is some variation in the abundance of natural isotopes in the synthesized compounds, depending on the sources of the chemical materials used in the synthesis. Therefore, the compound of the present invention will inherently contains small amounts of deuterated isotopologues. Despite this variation, the concentration of such naturally abundant stable hydrogen and carbon isotopes is very low and inconsequential compared to the degree to which the compound of the present invention is substituted with stable isotopes. See, e.g., Wada, E et. al., Seikagaku, 1994, 66: 15; and Gannes, L Z et. al., Comp BiochemPhysiol Mol IntegrPhysiol, 1998, 119: 725.

In the compound of the present invention, any atom not designated as deuterium is present at its natural isotopic abundance level. Unless otherwise stated, when “H” or “hydrogen” is specifically designated to a position, that position should be construed as having hydrogen composed of isotopes according to their natural abundance levels. Likewise, unless otherwise stated, when “D” or “deuterium” is specifically designated to a position, that position should be construed as having deuterium at an abundance level that is higher than the natural abundance level of deuterium (0.015%) by at least 3000 times (that is, at least 45% deuterium is incorporated).

The term “isotopic enrichment factor” as used herein refers to a ratio between the isotopic abundance and the natural abundance of a particular isotope.

In other embodiments, the isotopic enrichment factor for each designated deuterium atom in the compound of the present invention is at least 3500 (52.5% deuterium is incorporated at each designated deuterium atom), at least 4000 (60% deuterium is incorporated), at least 4500 (67.5% deuterium is incorporated), at least 5000 (75% deuterium is incorporated), at least 5500 (82.5% deuterium is incorporated), at least 6000 (90% deuterium is incorporated), at least 6333.3 (95% deuterium is incorporated), at least 6466.7 (97% deuterium is incorporated), at least 6600 (99% deuterium is incorporated), or at least 6633.3 (99.5% deuterium is incorporated).

The term “isotopologue” refers to a substance of which the chemical structure differs from that of a particular compound of the present invention only in isotopic composition.

The term “pharmaceutically acceptable salt” includes both acid addition salts and base addition salts.

The term “acid addition salts” includes, but is not limited to, salts derived from inorganic acids, such as hydrochloric acid, nitric acid, phosphoric acid, sulfuric acid, hydrobromic acid, hydroiodic acid, and phosphonic acid, and salts derived from organic acids, such as aliphatic mono-carboxylic acid and aliphatic dicarboxylic acid, phenyl-substituted alkanoic acid, hydroxyalkanoic acid, alkanedioic acids, aromatic acid, aliphatic sulfonic acid and aromatic sulfonic acid. Thus, these salts include, but are not limited to, sulfate, pyrosulfate, bisulfate, sulfite, bisulfite, nitrate, phosphate, monohydrogenphosphate, dihydrogenphosphate, metaphosphate, pyrophosphate, hydrochloride, hydrobromide, iodate, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, tosylate, phenylacetate, citrate, lactate, maleate, tartrate, and methanesulfonate, and salts comprising amino acids such as arginate, gluconate and galacturonate. Acid addition salts can be prepared by contacting the free base form with a sufficient amount of the desired acid to form the salt in a conventional manner. The free base form can be regenerated by contacting the salt form with a base and isolating the free base in a conventional manner.

The term “base addition salts” refers to salts formed with metals or amines, such as hydroxides of alkali metals and alkaline earth metals, or with organic amines. Examples of metals useful as cations include, but are not limited to, sodium, potassium, magnesium, and calcium. Examples of suitable amines include, but are not limited to, N,N-dibenzylethylenediamine, chloroprocaine, choline, diethanolamine, ethylenediamine(ethane-1,2-diamine), N-methylglucamine and procaine. Base addition salts can be prepared by contacting the free acid form with a sufficient amount of the desired base to form the salt in a conventional manner. The free acid form can be regenerated by contacting the salt form with an acid and isolating the free acid in a conventional manner.

The term “stereoisomer” includes enantiomeric, diastereomeric and geometric isomer forms. Some of the compounds of the present invention have cyclohydrocarbyl which may be substituted on more than one carbon atom, in which case all geometric forms thereof, including cis and trans, and mixtures thereof, are within the scope of the present invention.

The term “solvate” refers to a physical association of the compound of the present invention with one or more solvent molecules. The physical association includes various degrees of ionic and covalent bonding, including hydrogen bonding. In some cases, the solvate can be isolated, for example, when one or more solvent molecules are incorporated into the crystal lattice of a crystalline solid. Solvates include both solution phases and isolatable solvates. Representative solvates include ethanolates, methanolates, and the like.

The term “prodrug” refers to forms of the compound of formula I (including acetals, esters, and zwitterions) which are suitable for administration to patients without undue toxicity, irritation, allergic response and the like, and which are effective for the intended use thereof. The prodrug is converted in vivo, e.g., by hydrolysis in blood, to give the parent compound.

The terms “patient” and “subject” and the like are used interchangeably herein and refer to any animal or cell thereof, whether in vitro or in situ, treated according to the method described herein. Specifically, the aforementioned animal includes mammals, for example, rats, mice, guinea pigs, rabbits, dogs, monkeys, or humans, particularly humans.

The term “treating” refers to preventing, curing, reversing, attenuating, alleviating, minimizing, suppressing, arresting, and/or stopping one or more clinical symptoms of a disease after its onset.

The term “preventing” refers to treatment to avoid, minimize, or make difficult the onset or progression of a disease prior to its onset.

The term “tumor” refers to an abnormal mass of tissue, wherein the growth of the mass exceeds and is uncoordinated with that of normal tissue. A tumor may be “benign” or “malignant”, depending on the following characteristics: degree of cell differentiation (including morphology and functionality), growth rate, local invasion and metastasis. “Benign tumors” are generally well differentiated, characterized by growing slower than malignant tumors and remaining limited to the site of origin. In addition, benign tumors do not have the ability to infiltrate, invade or metastasize to distant sites. In some cases, certain “benign” tumors may later result in malignant tumors, possibly due to additional genetic alterations in subpopulations of neoplastic cells of the tumors, and these tumors are referred to as “precancerous tumors”. “Malignant tumors” are usually poorly differentiated (anaplastic), characterized by rapid growth and accompanied by progressive infiltration, invasion and destruction of surrounding tissues. In addition, malignant tumors generally have the ability to metastasize to distant sites. The term “cancer” refers to malignant tumors (Stedman's Medical Dictionary, 25th ed.; Hensyl ed.; Williams & Wilkins: Philadelphia, 1990). Exemplary cancers include, but are not limited to, adrenal cancer; anal cancer; angiosarcomas (e.g., lymphangiosarcoma, lymphangioendotheliosarcoma, angiosarcomas); appendiceal cancer; biliary tract cancer (e.g., cholangiocarcinoma); bladder cancer; breast cancer (e.g., breast adenocarcinoma, breast papillary carcinoma, mammary cancer, or breast medullary carcinoma); brain cancer (e.g., meningioma, glioblastoma, glioma (e.g., astrocytoma, and oligodendroglioma), and medulloblastoma); bronchial cancer; carcinoid tumor; cervical cancer (e.g., cervical adenocarcinoma); choriocarcinoma; chordoma; craniopharyngioma; colorectal cancer (e.g., colon cancer, rectal cancer, and colorectal adenocarcinoma); connective tissue cancer; epithelial cancer; ependymoma; endothelial sarcoma (e.g., Kaposi's sarcoma, and multiple idiopathic hemorrhagic sarcoma); endometrial cancer (e.g., uterine cancer, and uterine sarcoma); esophageal cancer (e.g., esophageal adenocarcinoma, and Barrett's adenocarcinoma); Ewing's sarcoma; eye cancer (e.g., intraocular melanoma, and retinoblastoma); gallbladder cancer; gastric cancer (e.g., gastric adenocarcinoma); gastrointestinal stromal tumor (GIST); germ cell cancer; head and neck cancer (e.g., head and neck squamous cell carcinoma, oral cavity cancer (e.g., oral cavity squamous cell carcinoma), and laryngeal cancer (e.g., laryngeal cancer, pharyngeal cancer, nasopharyngeal cancer, and oropharyngeal cancer)); hematologic malignancies (e.g., leukemias such as acute lymphocytic leukemia (ALL)(e.g., B-cell ALL, and T-cell ALL), acute myelogenous leukemia (AML)(e.g., B-cell AML, and T-cell AML), chronic myelogenous leukemia (CML)(e.g., B-cell CML, and T-cell CML), and chronic lymphocytic leukemia (CLL)(e.g., B-cell CLL, and T-cell CLL)); lymphomas, such as Hodgkin lymphoma (HL)(e.g., B-cell HL, and T-cell HL) and non-Hodgkin lymphoma (NHL)(e.g., B-cell NHL such as diffuse large cell lymphoma (DLCL)(e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, and splenic marginal zone B-cell lymphoma), primary mediastinal B-cell lymphoma, Burkitt lymphoma, and lymphoplasmacytic lymphoma (i.e., Waldenström's macroglobulinemia)), hairy cell leukemia (HCL), immunoblastic large cell lymphoma, precursor B lymphoblastic lymphoma and primary central nervous system (CNS) lymphoma; and T-cell NHL, such as precursor T-lymphoblastic lymphoma/leukemia, peripheral T-cell lymphoma (PTCL)(e.g., cutaneous T-cell lymphoma (CTCL)(e.g., mycosis fungoides, and Sezary syndrome), angioimmunoblastic T-cell lymphoma, extranodal natural killer T-cell lymphoma, enteropathy-associated T-cell lymphoma, subcutaneous panniculitis-like T-cell lymphoma and anaplastic large-cell lymphoma); a mixture of one or more of the leukemias/lymphomas described above; and multiple myeloma (MM), hemangioblastoma; hypopharyngeal carcinoma; inflammatory myofibroblastic tumor; immune cell amyloidosis; kidney cancer (e.g., nephroblastoma also known as Wilms' tumor, and renal cell carcinoma); liver cancer (e.g., hepatocellular carcinoma (HCC), and malignant hepatoma); lung cancer (e.g., bronchopulmonary carcinoma, small cell lung cancer (SCLC), non-small cell lung cancer (NSCLC), and lung adenocarcinoma); leiomyosarcoma (LMS); mastocytosis (e.g., systemic mastocytosis); muscle cancer; mesothelioma; myeloproliferative disease (MPD)(e.g., polycythemia vera (PV), essential thrombocythemia (ET), agnogenic myeloid metaplasia (AMM) also known as myelofibrosis (MF), chronic idiopathic myelofibrosis, chronic myelogenous leukemia (CML), chronic neutrophilic leukemia (CNL), and hypereosinophilic syndrome (HES)); neuroblastoma; neurofibromas (e.g., neurofibromatosis type 1 or type 2 (NF), and schwannomas); neuroendocrine cancer (e.g., gastroenteropancreatic neuroendocrine tumor (GEP-NET), and carcinoid tumor); osteosarcoma (e.g., bone cancer); ovarian cancer (e.g., cystadenocarcinoma, ovarian embryonal carcinoma, and ovarian adenocarcinoma); papillary adenocarcinoma; pancreatic cancer (e.g., pancreatic cancer, intraductal papillary mucinous neoplasm (IPMN), pancreatic islet cell tumor); and penile cancer (e.g., Paget's disease of the penis and scrotum); pineal gland tumors; primitive neuroectodermal tumor (PNT); prostate cancer (e.g., prostate adenocarcinoma); rhabdomyosarcoma; salivary gland cancer; skin cancer (e.g., squamous cell carcinoma (SCC), keratoacanthoma (KA), melanoma, and basal cell carcinoma (BCC)); small bowel cancer (e.g., appendiceal cancer); soft tissue sarcomas (e.g., malignant fibrous histiocytoma (MFH), liposarcoma, malignant peripheral nerve sheath tumor (MPNST), chondrosarcoma, fibrosarcoma, and myxosarcoma); sebaceous gland cancer; sweat gland cancer; synovial tumor; testicular cancer (e.g., seminoma, and testicular embryoma); thyroid cancer (e.g., papillary carcinoma of the thyroid, papillary thyroid cancer (PTC), and medullary thyroid cancer); urinary tract cancer; vaginal cancer; and vulvar cancer (e.g., Paget's disease of the vulva).

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, in which one or more of the carbons has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O, P and N may be placed at any interior position of the heteroalkyl group or at the position at which the alkyl group is attached to the remainder of the molecule. When the heteroalkyl is said to be substituted, the substituent(s) can replace one or more hydrogen atoms attached to the carbon atom(s) and/or the heteroatom(s) of the heteroalkyl. In some embodiments, the heteroalkyl is a C₁₋₄ heteroalkyl, which refers to the heteroalkyl defined herein having 1-4 carbon atoms. Examples of C₁₋₄ heteroalkyl include, but are not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, —CH₂—CH₂—S(O)₂—CH₃, C₂ heteroalkyl such as —CH₂—CH₂—OH, —CH₂—CH₂—NH₂, —CH₂—NH(CH₃), —O—CH₂—CH₃ and C₁ heteroalkyl such as, —CH₂—OH, —CH₂—NH₂, —O—CH₃. Preferably, the C₁₋₄ heteroalkyl (or C₁₋₄ heteroalkylene) herein contains 1 or 2 heteroatoms, such as one oxygen, one nitrogen, two oxygens, two nitrogens, or one oxygen and one nitrogen. In some preferred embodiments, unless specified or otherwise contrary, any of the C₁₋₄ heteroalkyl (or C₁₋₄ heteroalkylene) herein can have one oxygen or one nitrogen at the chain termini position, such as C₁₋₄ alkoxy, NH(C₁₋₄ alkyl), or a dialkylamino having a total of four carbons. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

As used herein, unless specified or otherwise contrary, a “ring structure”, “cyclic structure”, or simply “ring”, with a designated number of ring members, such as a “3-10 membered ring structure”, a “3-12 membered ring structure”, or a “5- or 6-membered ring”, should be understood as encompassing any ring structure (e.g., carbocyclic, heterocyclic, aryl, heteroaryl, etc.) having the designated number of ring members, which can be (1) monocyclic or polycyclic (as chemically feasible), such as a monocyclic ring or a bicyclic ring (including fused, spiro, and bridged bicyclic ring, and those ring systems where two monocyclic rings are connected through a single or double bond); (2) aromatic, partially unsaturated, or fully saturated; and in the case of a polycyclic structure, each ring can be independently aromatic, partially unsaturated, or fully saturated; and (3) containing no heteroatom (i.e., all ring members are carbon atoms) or 1-4 heteroatoms; in the case of a polycyclic structure, each ring can independently have no ring heteroatom or 1-4 ring heteroatoms (e.g., O, N, S, etc.). When a ring is said to contain a ring sulfur or nitrogen atom, the sulfur or nitrogen atom can be optionally oxidized. One or more ring carbon atoms in a ring structure can be present as C(═O). A fully saturated ring refers to a ring in which none of the ring carbon atom(s) and ring heteroatom(s) (e.g., nitrogen), if present, forms a double bond or triple bond with any other atom. The ring structure can be optionally substituted with one or more substituents described herein. The substituents of a ring structure herein can also have a cyclic structure, and in some cases, two substituents of a ring structure may be said to be joined to form a cyclic structure.

Unless otherwise defined or contrary from context, a heteroatom herein refers to an atom selected from nitrogen, oxygen, sulfur, boron, phosphorus, and silicon.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable, each of which can be optionally isotopically labeled, such as deuterated. Two of the optional substituents can join to form a ring structure, such as an optionally substituted cycloalkyl, heterocyclyl, aryl, or heteroaryl ring. Substitution can occur on any available carbon, oxygen, or nitrogen atom, and can form a spirocycle. Typically, substitution herein does not result in an O—O, O—N, S—S, S—N(except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y¹)(Y²), -alkylene-N(Y¹)(Y²), —C(O)N(Y¹)(Y²) and —S(O)₂N(Y¹)(Y²), wherein Y¹ and Y² can be the same or different and are independently selected from the group consisting of hydrogen, alkyl, aryl, cycloalkyl, and -alkylene-aryl.

Some examples of suitable substituents include, but not limited to, (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, —NHC(O)H, —NHC(O)(C₁-C₈)alkyl groups, —NHC(O)(C₃-C₈)cycloalkyl groups, —N((C₁₋₈)alkyl)C(O)H, —N((C₁-C₈)alkyl)C(O)(C₁-C₈)alkyl groups, —NHC(O)NH₂, —NHC(O)NH(C₁-C₈)alkyl groups, N((C₁-C₈)alkyl)C(O)NH₂ groups, —NHC(O)N((C₁₋₈)alkyl)₂ groups, —N((C₁-C₈)alkyl)C(O)N((C₁-C₈)alkyl)₂ groups, —N((C₁₋₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, —N((C₁-C₈)alkyl)S(O)₂(C₁-C₈)alkyl groups, (C₁-C₈)alkyl-O—(C₁-C₈)alkyl groups, —O—(C₁-C₈)alkyl-O—(C₁-C₈)alkyl groups, —C(O)OH, —C(O)O(C₁₋₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O —(C₁-C₆)alkyl groups, —C(═NH)—(C₁-C₆)alkyl groups, —C(═NOH)(C₁-C₆)alkyl groups, or —C(═N—O—(C₁-C₆)alkyl)-(C₁-C₆)alkyl groups.

Exemplary carbon atom substituents include, but are not limited to, deuterium, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂(C₁₋₆ alkyl), —OC(═O)(C₁₋₆ alkyl), -OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3rd edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trimethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

The disclosures of the various publications, patents, and published patent specifications cited herein are hereby incorporated by reference in their entirety. To the extent that any meaning or definition of a term in this document conflicts with any meaning or definition of the same term in a document incorporated by reference, the meaning or definition assigned to that term in this document shall govern.

The technical schemes of the present invention will be clearly and completely described below with reference to the examples of the present invention, and it is obvious that the described examples are only a part of the examples of the present invention but not all of them. Based on the examples of the present invention, all other examples obtained by those of ordinary skill in the art without creative work shall fall within the protection scope of the present invention.

### SYNTHESIS EXAMPLES

### Example 1

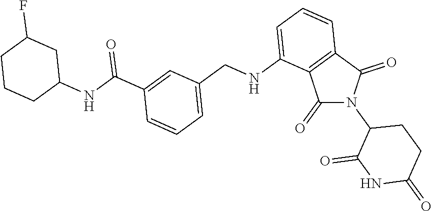 [SMILES: O=C1CCC(N2C(=O)c3cccc(NCc4cccc(C(=O)NC5CCCC(F)C5)c4)c3C2=O)C(=O)N1]

### Preparation Steps

### Step 1

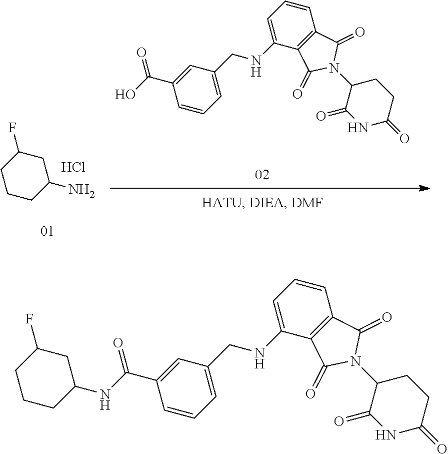 [SMILES: NC1CCCC(F)C1 ; Cl ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCCC(F)C5)c4)c3C2=O)C(=O)N1]

01 (29 mg, 0.189 mmol) was dissolved in N,N-dimethylformamide (1 mL) at room temperature, and 02 (50 mg, 0.123 mmol), HATU (70 mg, 0.184 mmol) and N,N-diisopropylethylamine (79 mg, 0.611 mmol) were added successively. The above mixture was stirred at room temperature for 1 h. The reaction mixture was purified by preparative high performance liquid chromatography to give a yellowish-green solid (43.66 mg, 70.2% yield). LC-MS: 507.3 [M+H]⁺, ¹H NMR (400 MHz, DMSO-d₆) δ 11.10 (s, 1H), 8.30 (dd, J=34.7, 8.0 Hz, 1H), 7.85 (s, 1H), 7.71 (d, J=7.7 Hz, 1H), 7.53-7.47 (m, 2H), 7.41 (t, J=7.6 Hz, 1H), 7.29 (t, J=6.2 Hz, 1H), 7.03 (d, J=7.1 Hz, 1H), 6.94 (d, J=8.6 Hz, 1H), 5.07 (dd, J=12.8, 5.4 Hz, 1H), 4.60 (d, J=6.1 Hz, 2H), 4.15-3.78 (m, 1H), 2.96-2.83 (m, 1H), 2.57 (dd, J=17.7, 10.5 Hz, 2H), 2.04 (d, J=5.4 Hz, 2H), 1.77 (dd, J=37.4, 20.0 Hz, 3H), 1.64-1.25 (m, 5H).

### Example 2

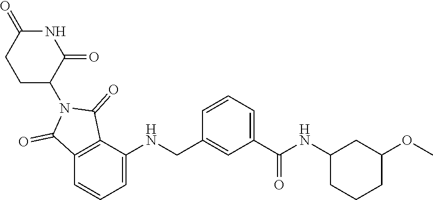 [SMILES: COC1CCCC(NC(=O)c2cccc(CNc3cccc4c3C(=O)N(C3CCC(=O)NC3=O)C4=O)c2)C1]

### Preparation Steps

### Step 1

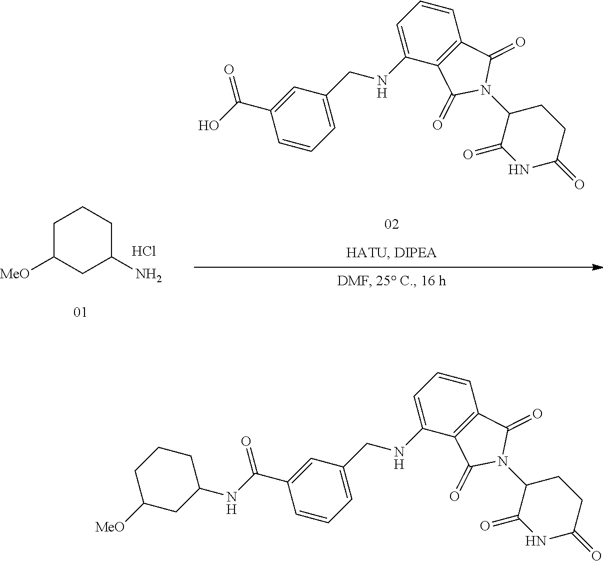 [SMILES: O=C1CCC(N2C(=O)c3cccc(NCc4cccc(C(=O)O)c4)c3C2=O)C(=O)N1 ; COC1CCCC(N)C1 ; Cl ; COC1CCCC(NC(=O)c2cccc(CNc3cccc4c3C(=O)N(C3CCC(=O)NC3=O)C4=O)c2)C1]

To a 100 mL single-neck flask were added 01 (30 mg, 0.181 mmol), 02 (73 mg, 0.181 mmol), HATU (103 mg, 0.272 mmol), N,N-diisopropylethylamine (70 mg, 0.543 mmol) and N,N-dimethylformamide (2 mL). The mixture was reacted at room temperature for 16 h and purified by preparative reversed-phase chromatography to give the target product as a yellow solid (65.9 mg, 70.2% yield). LC-MS: 519.1 [M+H]⁺, ¹H NMR (400 MHz, DMSO-d₆) δ 11.09 (s, 1H), 8.25 (d, J=4.0 Hz, 1H), 8.14 (d, J=4.0 Hz, 1H), 7.84 (s, 1H), 7.70 (d, J=7.0 Hz, 1H), 7.55-7.45 (m, 2H), 7.40 (td, J=7.6, 3.0 Hz, 1H), 7.27 (t, J=6.1 Hz, 1H), 7.03 (d, J=7.1 Hz, 1H), 6.94 (d, J=8.6 Hz, 1H), 5.07 (dd, J=12.9, 5.4 Hz, 1H), 4.60 (d, J=6.1 Hz, 2H), 4.14-3.99 (m, 1H), 3.79 (s, 1H), 3.23 (d, J=4.8 Hz, 3H), 2.95-2.83 (m, 1H), 2.64-2.54 (m, 2H), 2.21-1.91 (m, 3H), 1.74 (s, 2H), 1.58-1.41 (m, 2H), 1.35-1.15 (m, 2H).

### Example 3

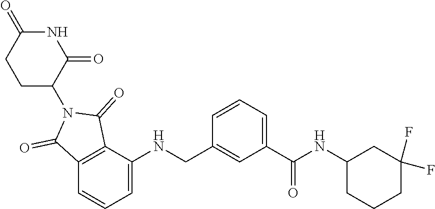 [SMILES: O=C1CCC(N2C(=O)c3cccc(NCc4cccc(C(=O)NC5CCCC(F)(F)C5)c4)c3C2=O)C(=O)N1]

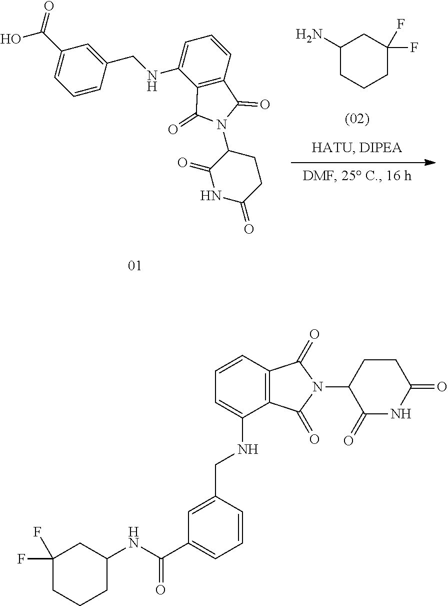 [SMILES: O=C1CCC(N2C(=O)c3cccc(NCc4cccc(C(=O)O)c4)c3C2=O)C(=O)N1 ; NC1CCCC(F)(F)C1 ; O=C1CCC(N2C(=O)c3cccc(NCc4cccc(C(=O)NC5CCCC(F)(F)C5)c4)c3C2=O)C(=O)N1]

01 (71 mg, 0.175 mmol) was dissolved in N,N-dimethylformamide (2 mL) at room temperature, and HATU (100 mg, 0.263 mmol), N,N-diisopropylethylamine (68 mg, 0.526 mmol) and 02 (30 mg, 0.175 mmol) were added successively. The mixture was stirred at room temperature for 16 h. The reaction mixture was purified by preparative high performance liquid chromatography to give the target product as a yellow solid (50.92 mg, 55.5% yield). LC-MS: 525.19 [M+H]⁺, ¹H NMR (400 MHz, DMSO-d₆) δ 11.10 (s, 1H), 8.39 (d, J=7.9 Hz, 1H), 7.85 (s, 1H), 7.71 (d, J=7.7 Hz, 1H), 7.51 (t, J=7.8 Hz, 2H), 7.42 (t, J=7.6 Hz, 1H), 7.30 (t, J=6.1 Hz, 1H), 7.03 (d, J=7.0 Hz, 1H), 6.94 (d, J=8.6 Hz, 1H), 5.07 (dd, J=13.0, 5.4 Hz, 1H), 4.61 (d, J=6.3 Hz, 2H), 4.00 (s, 1H), 2.95-2.84 (m, 1H), 2.60-2.51 (m, 2H), 2.29-2.23 (m, 1H), 2.04 (d, J=7.4 Hz, 2H), 1.88-1.77 (m, 3H), 1.41-1.25 (m, 2H).

### Example 4

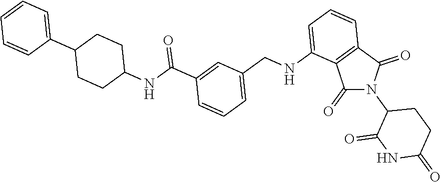 [SMILES: O=C1CCC(N2C(=O)c3cccc(NCc4cccc(C(=O)NC5CCC(c6ccccc6)CC5)c4)c3C2=O)C(=O)N1]

### Preparation Steps

### Step 1

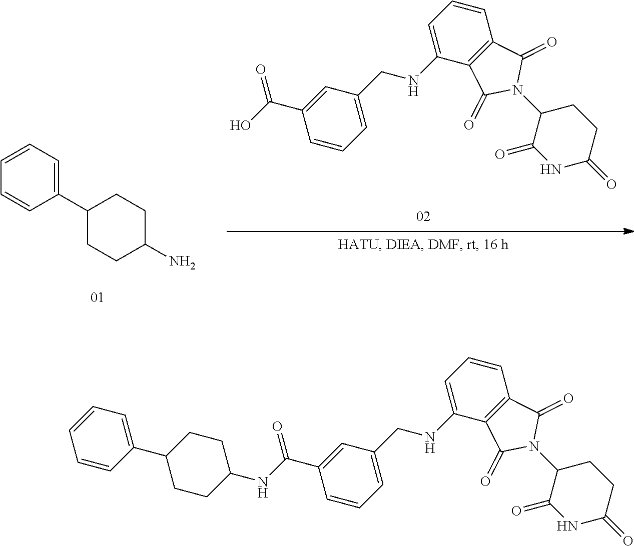 [SMILES: NC1CCC(c2ccccc2)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6ccccc6)CC5)c4)c3C2=O)C(=O)N1]

02 (30.0 mg, 0.074 mmol) was dissolved in N,N-dimethylformamide (1 mL) at room temperature, and 01 (13.0 mg, 0.074 mmol), HATU (42.2 mg, 0.111 mmol) and N,N-diisopropylethylamine (28.7 mg, 0.222 mmol) were added successively. The above mixture was stirred at room temperature for 16 h. The reaction mixture was purified by preparative high performance liquid chromatography to give a yellowish-green solid (22.7 mg, 54.6% yield). LC-MS: 565.2 [M+H]⁺, ¹H NMR (400 MHz, DMSO-d₆) δ 11.09 (s, 1H), 8.19 (dd, J=50.5, 7.9 Hz, 1H), 7.85 (d, J=17.0 Hz, 1H), 7.73 (d, J=7.5 Hz, 1H), 7.54-7.48 (m, 2H), 7.42 (dt, J=10.7, 5.3 Hz, 1H), 7.32-7.25 (m, 5H), 7.18 (d, J=6.3 Hz, 1H), 7.03 (dd, J=6.9, 3.3 Hz, 1H), 6.96 (dd, J=8.5, 3.4 Hz, 1H), 5.07 (dd, J=12.7, 5.1 Hz, 1H), 4.62 (s, 2H), 3.85 (s, 1H), 2.89 (s, 1H), 2.56 (s, 2H), 1.95 (dd, J=50.7, 38.5 Hz, 6H), 1.69-1.47 (m, 4H).

### Example 5

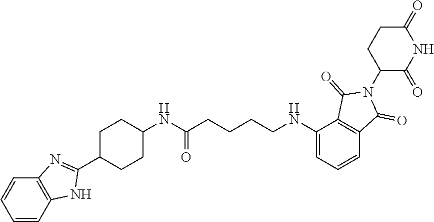 [SMILES: O=C1CCC(N2C(=O)c3cccc(NCCCCC(=O)NC4CCC(c5nc6ccccc6[nH]5)CC4)c3C2=O)C(=O)N1]

### Preparation Steps

### Step 1

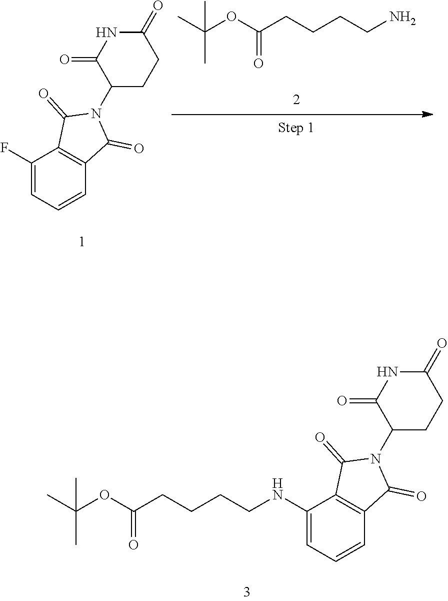

1 (200 mg, 0.724 mmol), 2 (150.5 mg, 0.87 mmol) and N-methylpyrrolidone (5 mL) were added to a 100 mL single-neck flask and mixed well by stirring, and DIEA (468 mg, 3.62 mmol) was added. The mixture was reacted at 110° C. for 18 h and concentrated to dryness by rotary evaporation, and the residue was purified by column chromatography to give the target product as a yellowish-green solid (171 mg, 55%). LC-MS: 430 [M+H]⁺.

### Step 2

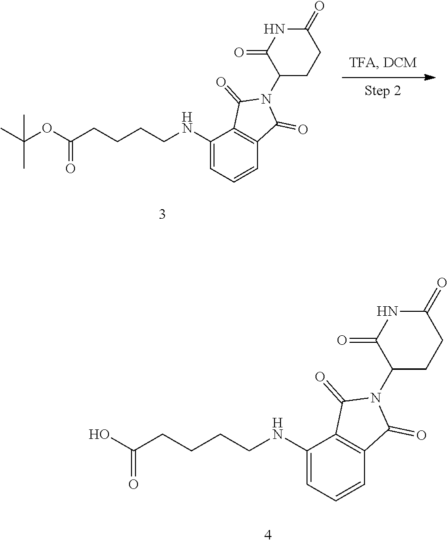 [SMILES: CC(C)(C)OC(=O)CCCCNc1cccc2c1C(=O)N(C1CCC(=O)NC1=O)C2=O ; O=C(O)CCCCNc1cccc2c1C(=O)N(C1CCC(=O)NC1=O)C2=O]

In a 100 mL single-neck flask, 3 (171 mg, 0.323 mmol) was dissolved in DCM (4 mL), and TFA (0.8 mL) was added. The mixture was reacted at room temperature for 16 h and concentrated to dryness by rotary evaporation, and the residue was purified by column chromatography to give the target product as a yellowish-green solid (135 mg, 91%). LC-MS: 374 [M+H]⁺.

### Step 3

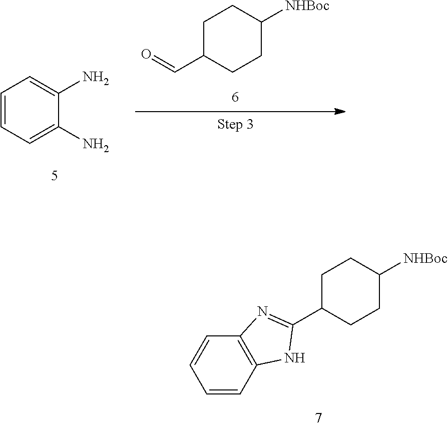 [SMILES: CC(C)(C)OC(=O)NC1CCC(C=O)CC1 ; Nc1ccccc1N ; CC(C)(C)OC(=O)NC1CCC(c2nc3ccccc3[nH]2)CC1]

5 (1 g, 9.25 mmol), 6 (3 g, 13.2 mmol) and methanol (100 mL) were added to a 250 mL single-neck flask and mixed well by stirring, and AcOH (8 mL) was added. The mixture was reacted in an oxygen atmosphere at 50° C. overnight and concentrated to dryness by rotary evaporation, and the residue was purified by column chromatography to give the target product as a white solid (440 mg, 15.1%). LC-MS: 316 [M+H]⁺.

### Step 4

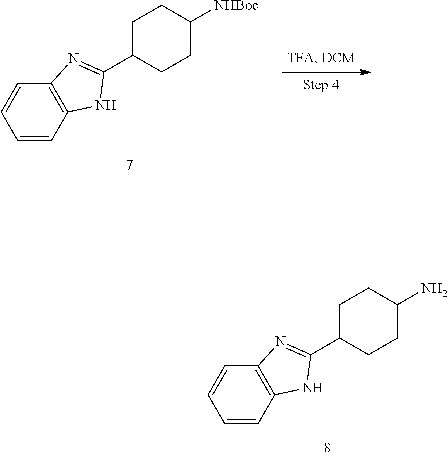 [SMILES: CC(C)(C)OC(=O)NC1CCC(c2nc3ccccc3[nH]2)CC1 ; NC1CCC(c2nc3ccccc3[nH]2)CC1]

In a 100 mL single-neck flask, 7 (440 mg, 1.395 mmol) was dissolved in DCM (4 mL), and TFA (0.8 mL) was added. The mixture was reacted at room temperature for 16 h and concentrated to dryness by rotary evaporation, and the residue was purified by column chromatography to give the target product as a yellowish-green solid (235 mg, 78.2%). LC-MS: 216 [M+H]⁺.

### Step 5

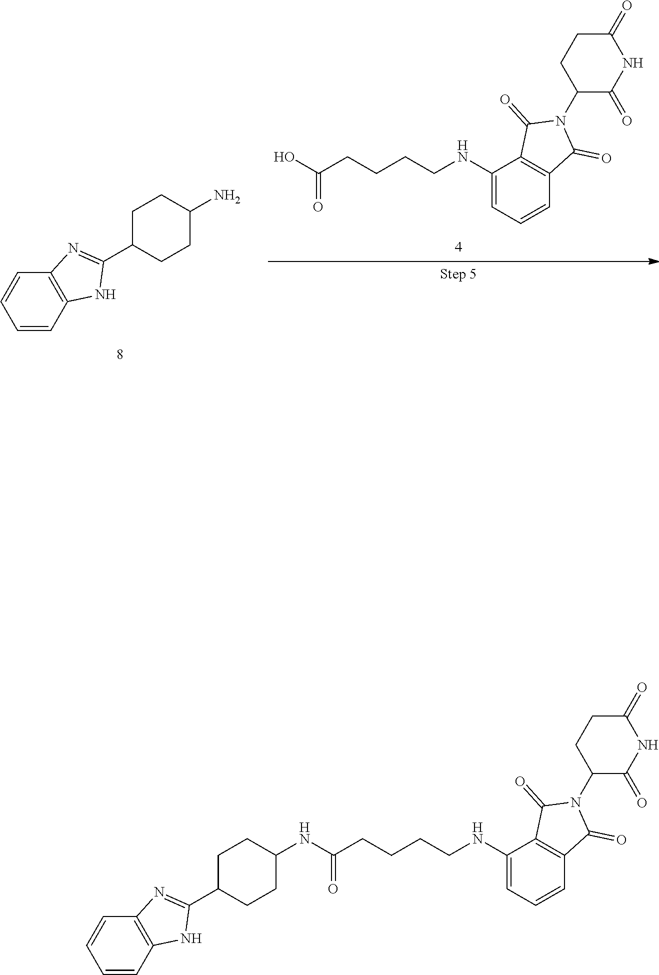 [SMILES: NC1CCC(c2nc3ccccc3[nH]2)CC1 ; O=C(O)CCCCNc1cccc2c1C(=O)N(C1CCC(=O)NC1=O)C2=O ; O=C1CCC(N2C(=O)c3cccc(NCCCCC(=O)NC4CCC(c5nc6ccccc6[nH]5)CC4)c3C2=O)C(=O)N1]

In a 100 mL single-neck flask, 4 (135 mg, 0.362 mmol) and 8 (93.4 mg, 0.434 mmol) were dissolved in DMF (4 mL) and mixed well by stirring, and HATU (206.5 mg, 0.543 mmol) and DIEA (140 mg, 1.086 mmol) were added. The mixture was reacted at room temperature for 18 h and concentrated to dryness by rotary evaporation, and the residue was purified by preparative high performance liquid chromatography to give the target product (17.3 mg, 8.38% yield). LC-MS: 571 [M+H]+¹H NMR (400 MHz, DMSO) δ 11.11 (s, 1H), 7.81 (m, 3H), 7.56 (m, 3H), 7.11 (d, J=8.6 Hz, 1H), 7.03 (d, J=7.0 Hz, 1H), 6.59 (s, 1H), 5.06 (dd, J=12.9, 5.3 Hz, 1H), 3.62 (m, 1H), 3.17 (t, J=12.1 Hz, 1H), 2.89 (dd, J=15.6, 10.5 Hz, 1H), 2.58 (m, 2H), 2.05 (d, J=37.6, 29.3, 15.3 Hz, 8H), 1.77 (m, 2H), 1.58 (s, 4H), 1.32 (m, 3H).

### Example 6

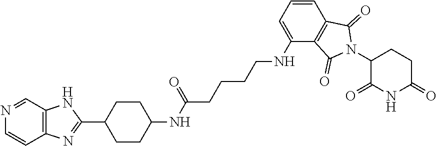 [SMILES: O=C1CCC(N2C(=O)c3cccc(NCCCCC(=O)NC4CCC(c5nc6ccncc6[nH]5)CC4)c3C2=O)C(=O)N1]

### Preparation Steps

### Step 1

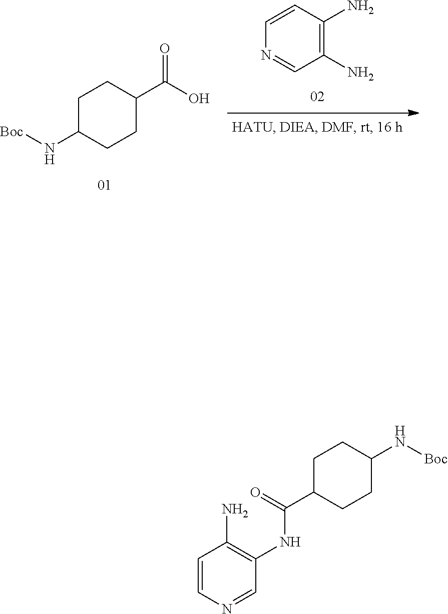 [SMILES: CC(C)(C)OC(=O)NC1CCC(C(=O)O)CC1 ; Nc1ccncc1N ; CC(C)(C)OC(=O)NC1CCC(C(=O)Nc2cnccc2N)CC1]

To a 500 mL single-neck flask were added 01 (7 g, 28.8 mmol), 02 (3.14 g, 28.8 mmol), HATU (16.4 g, 43.1 mmol), DIEA (11.15 g, 86.3 mmol) and DMF (200 mL), and the mixture was reacted at room temperature for 16 h. Water (300 mL) was added, followed by extraction with ethyl acetate (40 mL). The organic phase was washed with saturated brine (200 mL×3), dried over anhydrous sodium sulfate, and concentrated to dryness by rotary evaporation, and the residue was purified by column chromatography (dichloromethane:methanol=10:1) to give the target product as a brown solid (5 g, 51.97% yield). LC-MS: 335.1 [M+H]⁺.

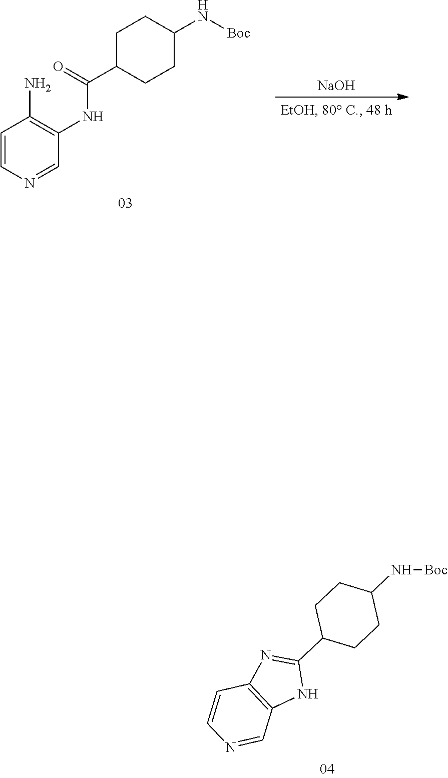 [SMILES: CC(C)(C)OC(=O)NC1CCC(C(=O)Nc2cnccc2N)CC1 ; "NaOH" ; CC(C)(C)OC(=O)NC1CCC(c2nc3ccncc3[nH]2)CC1]

In a 100 mL single-neck round-bottom flask, 03 (5 g, 15.0 mmol) and NaOH (1.79 g, 44.8 mmol) were dissolved in ethanol (50 mL), and the mixture was reacted at 80° C. for 48 h. The reaction mixture was concentrated to dryness by rotary evaporation, and water (50 mL) was added, followed by extraction with ethyl acetate (40 mL). The organic phase was washed with saturated brine (30 mL×3), dried over anhydrous sodium sulfate, and concentrated to dryness by rotary evaporation, and the residue was purified by column chromatography (dichloromethane:methanol=10:1) to give the target product as a pale yellow solid (1.3 g, 27.48% yield). LC-MS: 317.1 [M+H]⁺.

### Step 3

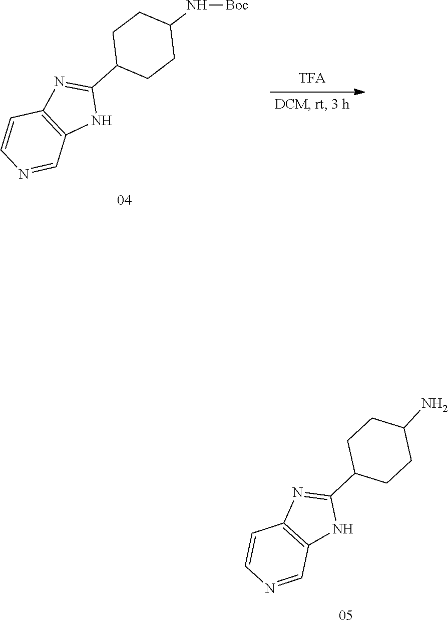 [SMILES: CC(C)(C)OC(=O)NC1CCC(c2nc3ccncc3[nH]2)CC1 ; NC1CCC(c2nc3ccncc3[nH]2)CC1]

In a 100 mL single-neck flask, 04 (100 mg, 0.316 mmol) was dissolved in dichloromethane:trifluoroacetic acid (4 mL, 3:1), and the mixture was stirred at room temperature for 2 h and purified by preparative reversed-phase liquid chromatography to give the target product as a brown solid (40 mg, 58.52% yield). LC-MS: 217.0 [M+H]⁺.

### Step 4

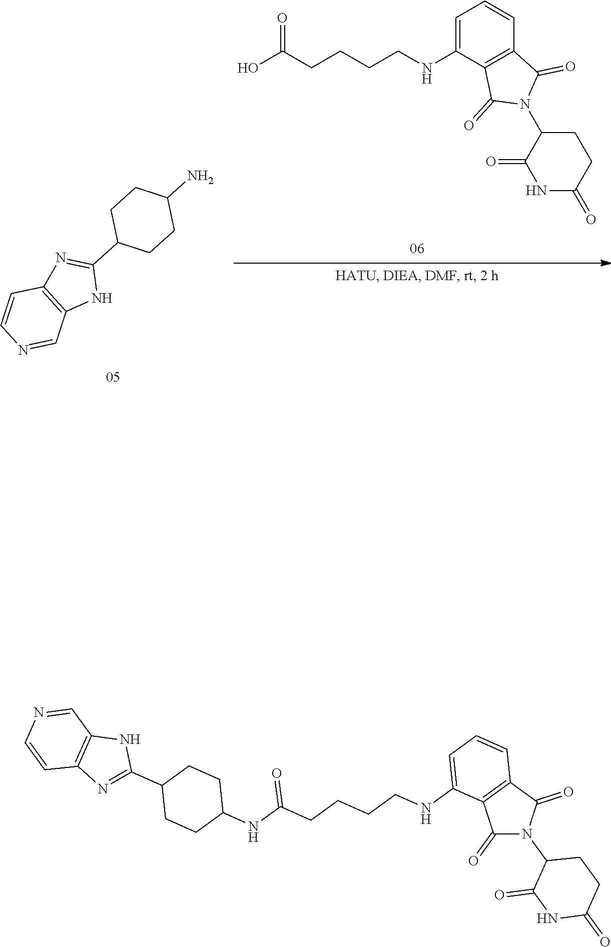 [SMILES: NC1CCC(c2nc3ccncc3[nH]2)CC1 ; O=C(O)CCCCNc1cccc2c1C(=O)N(C1CCC(=O)NC1=O)C2=O ; O=C1CCC(N2C(=O)c3cccc(NCCCCC(=O)NC4CCC(c5nc6ccncc6[nH]5)CC4)c3C2=O)C(=O)N1]

To a 100 mL single-neck flask were added 05 (40 mg, 0.185 mmol), 06 (70 mg, 0.187 mmol), HATU (105 mg, 0.276 mmol), DIEA (72 mg, 0.557 mmol) and DMF (2 mL), and the mixture was reacted at room temperature for 16 h and purified by preparative reversed-phase liquid chromatography to give the target product as a yellowish-green solid (11.44 mg, 10.82% yield). LC-MS: 572.3 [M+H]⁺, ¹H NMR (400 MHz, DMSO-d₆) δ 12.78 (s, 1H), 11.10 (d, J=8.8 Hz, 1H), 10.84-10.81 (m, 1H), 8.90 (s, 1H), 8.29 (s, 1H), 7.74 (dd, J=12.8, 7.9 Hz, 1H), 7.65-7.51 (m, 2H), 7.10 (d, J=8.7 Hz, 1H), 7.03 (d, J=7.0 Hz, 1H), 6.57 (t, J=5.8 Hz, 1H), 5.05 (dd, J=12.8, 5.4 Hz, 1H), 3.61 (d, J=4.5 Hz, 1H), 3.34 (s, 2H), 2.86 (d, J=9.3 Hz, 2H), 2.62-2.54 (m, 2H), 2.10 (d, J=6.9 Hz, 3H), 2.02 (d, J=7.7 Hz, 1H), 1.91 (d, J=9.6 Hz, 3H), 1.68-1.57 (m, 6H), 1.37-1.29 (m, 2H).

### Example 7

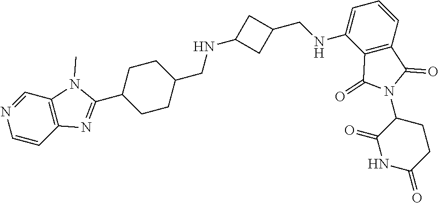 [SMILES: Cn1c(C2CCC(CNC3CC(CNc4cccc5c4C(=O)N(C4CCC(=O)NC4=O)C5=O)C3)CC2)nc2ccncc21]

### Preparation Steps

### Step 1

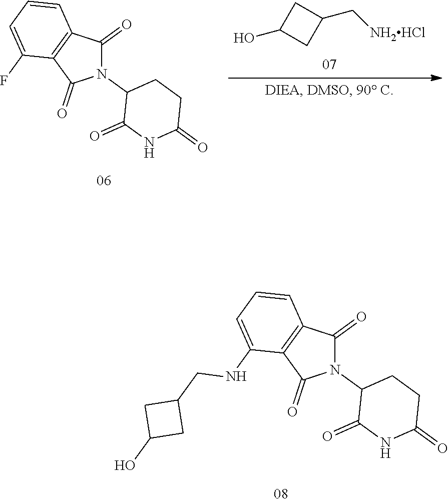

06 (700 g, 2.53 mmol), 07 (418 mg, 3.04 mmol) and N,N-diisopropylethylamine (982 mg, 7.60 mmol) were mixed in anhydrous dimethyl sulfoxide (5 mL). The reaction mixture was stirred at 90° C. for 16 h, cooled to 20° C., and then directly purified through a reversed-phase column (C18, 10-100% acetonitrile/water as mobile phase, 0.1% formic acid) to give the target product as a brownish-yellow solid (600 mg, 66.3% yield). LC-MS: 358.1 [M+H]⁺; 380.1 [M+Na]⁺.

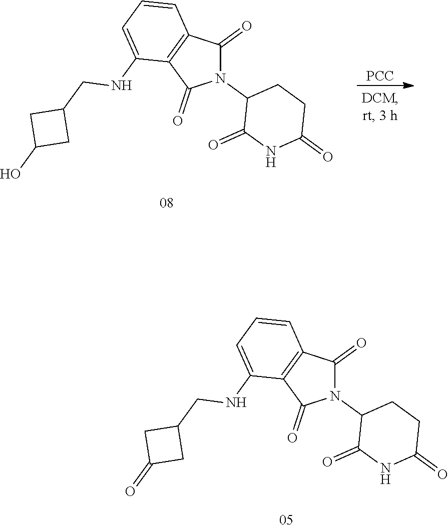 [SMILES: O=C1CCC(N2C(=O)c3cccc(NCC4CC(O)C4)c3C2=O)C(=O)N1 ; "PCC" ; O=C1CC(CNc2cccc3c2C(=O)N(C2CCC(=O)NC2=O)C3=O)C1]

08 (600 mg, 1.68 mmol) was dissolved in anhydrous dichloromethane (10 mL) at room temperature, and then pyridinium chlorochromate (724 mg, 3.36 mmol) was added. The mixture was stirred at room temperature for 3 h and passed through a silica gel column (dichloromethane:methanol=10:1) to separate the target product as a yellow solid (400 mg, 67.0% yield). LC-MS: 356.3 [M+H]⁺.

### Step 3

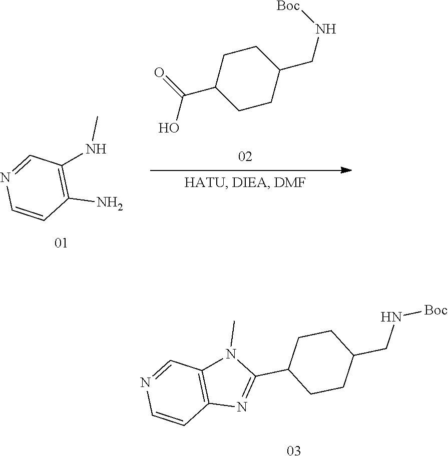

01 (750 mg, 6.09 mmol), 02 (1.57 g, 6.09 mmol) and N,N-diisopropylethylamine (2.36 g, 18.3 mmol) were mixed in anhydrous N,N-dimethylformamide (10 mL) at room temperature, and (7-azabenzotriazole)-tetramethyluronium hexafluorophosphate (3.47 g, 9.14 mmol) was added with stirring at room temperature. The reaction mixture was stirred at room temperature for 2 h. The mixture was passed through a reversed-phase column (C18, 10-100% acetonitrile/water as mobile phase, 0.1% formic acid) to separate the target product as a white solid (700 mg, 33.4% yield). LC-MS: [M+H]⁺.

### Step 4

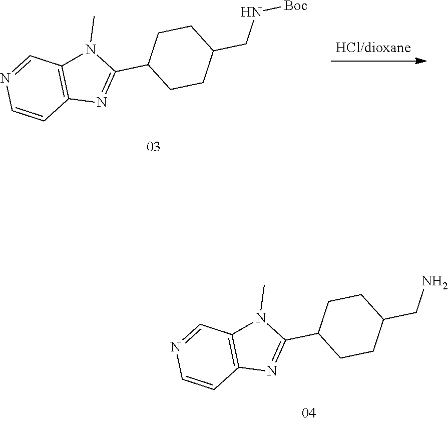 [SMILES: Cn1c(C2CCC(CNC(=O)OC(C)(C)C)CC2)nc2ccncc21 ; Cn1c(C2CCC(CN)CC2)nc2ccncc21]

03 (160 mg, 0.464 mmol) was dissolved in dichloromethane (2 mL) at room temperature, followed by dropwise addition of a solution of hydrogen chloride in dioxane (2 mL). The reaction mixture was stirred at room temperature for 2 h and concentrated to dryness by rotary evaporation to give the target product as a white solid (167 mg, crude). LC-MS: 245.1 [M+H]⁺.

### Step 5

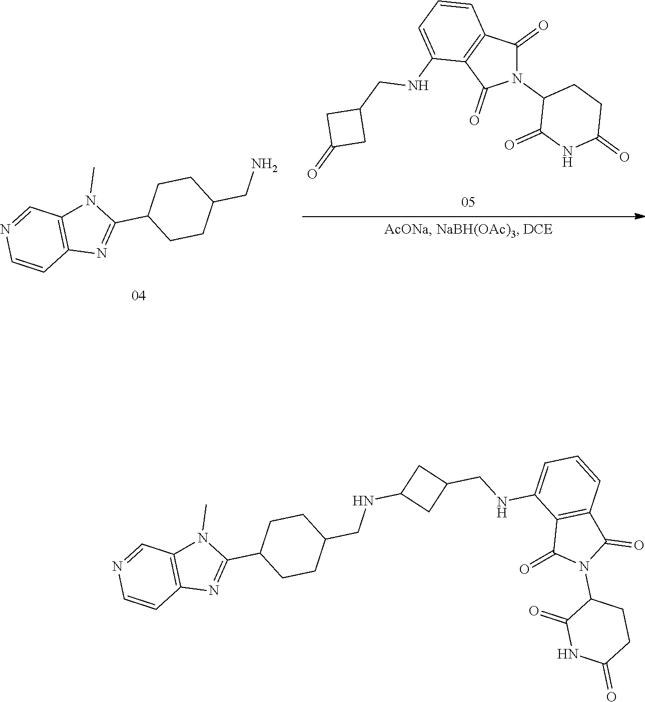 [SMILES: Cn1c(C2CCC(CN)CC2)nc2ccncc21 ; O=C1CC(CNc2cccc3c2C(=O)N(C2CCC(=O)NC2=O)C3=O)C1 ; Cn1c(C2CCC(CNC3CC(CNc4cccc5c4C(=O)N(C4CCC(=O)NC4=O)C5=O)C3)CC2)nc2ccncc21]

04 (20.0 mg, 0.071 mmol), 05 (25.2 mg, 0.071 mmol) and sodium acetate (19.3 mg, 0.142 mmol) were dissolved in dichloroethane (2 mL) at room temperature, and the mixture was stirred at room temperature for 30 min, followed by addition of sodium triacetoxyborohydride (45.1 mg, 0.213 mmol). The reaction mixture was stirred at room temperature for 16 h. The mixture was purified by preparative chromatography (C18, 5-100% acetonitrile/water as mobile phase, 0.1% formic acid) to separate the target product as a green solid (7.80 mg, 18.7% yield). LC-MS: 584.3 [M+H]⁺, ¹H NMR (400 MHz, DMSO-d₆) δ 11.15 (s, 1H), 8.92 (s, 1H), 8.34-8.31 (m, 2H), 7.67-7.57 (m, 2H), 7.22-7.14 (m, 1H), 7.11-7.06 (m, 1H), 6.65-6.53 (m, 1H), 5.11 (dd, J=12.4, 5.3 Hz, 1H), 3.93-3.89 (m, 3H), 3.47-3.44 (m, 1H), 3.41-3.37 (m, 1H), 3.25-3.19 (m, 1H), 3.08-2.88 (m, 2H), 2.68-2.59 (m, 2H), 2.53-2.43 (m, 2H), 2.39-2.22 (m, 2H), 2.10-1.84 (m, 6H), 1.83-1.50 (m, 6H), 1.22-1.11 (m, 1H).

### Example 8

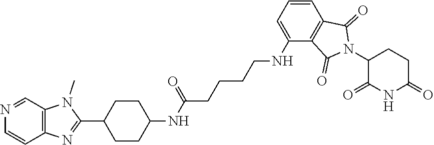 [SMILES: Cn1c(C2CCC(NC(=O)CCCCNc3cccc4c3C(=O)N(C3CCC(=O)NC3=O)C4=O)CC2)nc2ccncc21]

### Preparation Steps

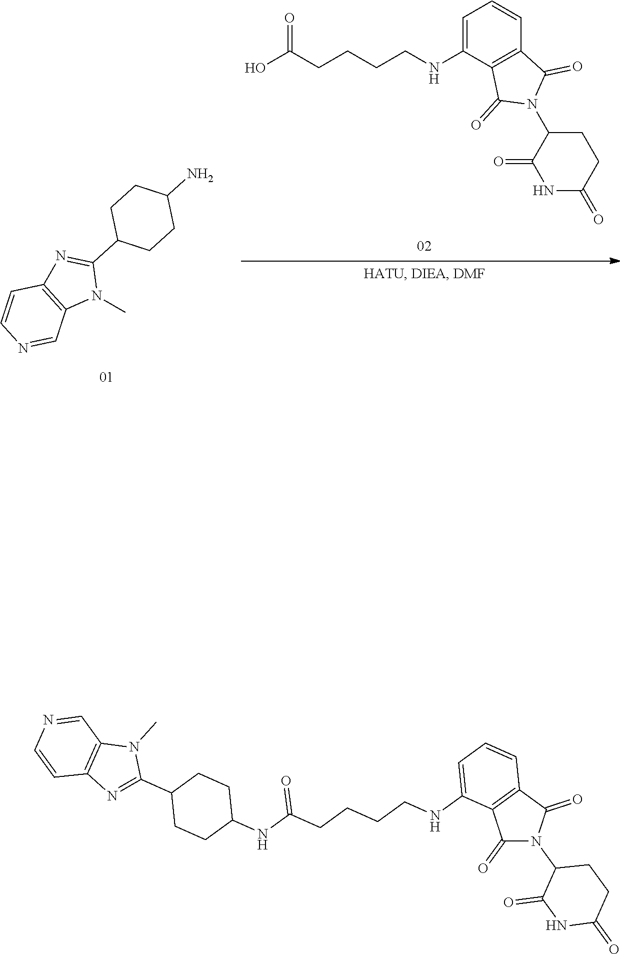 [SMILES: Cn1c(C2CCC(N)CC2)nc2ccncc21 ; O=C(O)CCCCNc1cccc2c1C(=O)N(C1CCC(=O)NC1=O)C2=O ; Cn1c(C2CCC(NC(=O)CCCCNc3cccc4c3C(=O)N(C3CCC(=O)NC3=O)C4=O)CC2)nc2ccncc21]

To a 100 mL single-neck flask were added 01 (30 mg, 0.130 mmol), 02 (49 mg, 0.131 mmol), HATU (74 mg, 0.195 mmol), DIEA (51 mg, 0.395 mmol) and DMF (1 mL), and the mixture was reacted at room temperature for 1 h and purified by preparative liquid chromatography to give the target product as a yellowish-green solid (51.59 mg, 67.6% yield). LC-MS: 586.4 [M+H]⁺.

¹H NMR (400 MHz, DMSO-d₆) δ 11.09 (s, 1H), 8.86 (s, 1H), 8.27 (d, J=5.5 Hz, 1H), 8.20 (s, 1H), 7.78 (d, J=7.5 Hz, 1H), 7.61-7.56 (m, 1H), 7.54 (d, J=5.4 Hz, 1H), 7.10 (d, J=8.7 Hz, 1H), 7.02 (d, J=7.0 Hz, 1H), 6.58 (s, 1H), 5.05 (dd, J=12.9, 5.4 Hz, 1H), 3.86 (d, J=4.1 Hz, 3H), 3.60 (s, 1H), 2.98 (t, J=11.8 Hz, 1H), 2.92-2.82 (m, 1H), 2.60 (dd, J=33.5, 16.9 Hz, 2H), 2.12 (s, 2H), 1.94 (dd, J=28.3, 11.6 Hz, 6H), 1.79-1.61 (m, 3H), 1.58 (s, 4H), 1.35 (dd, J=23.7, 10.8 Hz, 2H).

### Example 9

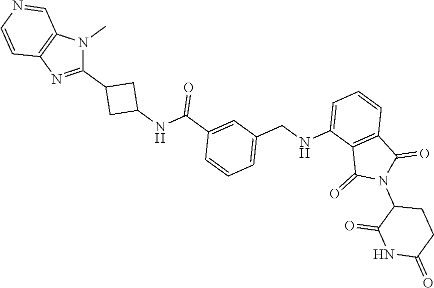 [SMILES: Cn1c(C2CC(NC(=O)c3cccc(CNc4cccc5c4C(=O)N(C4CCC(=O)NC4=O)C5=O)c3)C2)nc2ccncc21]

### Preparation Steps

### Step 1

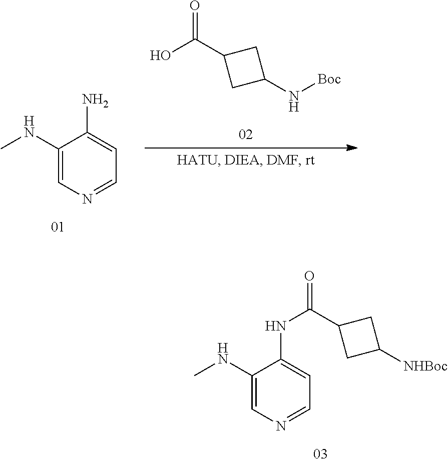

To a 100 mL single-neck flask were added 01 (400 mg, 3.25 mmol), 02 (664 mg, 3.08 mmol), HATU (1.85 g, 4.87 mmol), N,N-diisopropylethylamine (1.26 g, 9.75 mmol) and N,N-dimethylformamide (20 mL), and the mixture was reacted at room temperature for 2 h, quenched with water (20 mL), and extracted with ethyl acetate (20 mL×2). The organic phase was washed with saturated brine (20 mL×3) and concentrated to dryness by rotary evaporation, and the residue was purified by column chromatography (dichloromethane:methanol=20:1) to give the target product as a yellow solid (300 mg, 28.8% yield). LC-MS: 321.2 [M+H]⁺.

### Step 2

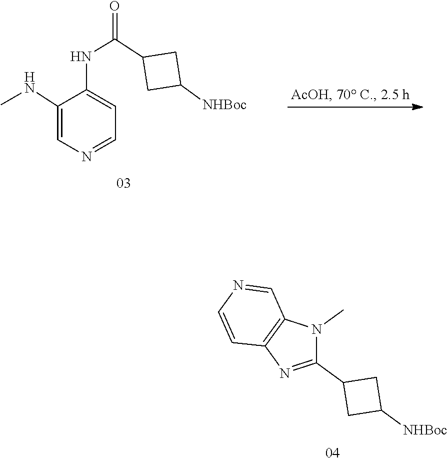 [SMILES: CNc1cnccc1NC(=O)C1CC(NC(=O)OC(C)(C)C)C1 ; Cn1c(C2CC(NC(=O)OC(C)(C)C)C2)nc2ccncc21]

In a 100 mL single-neck round-bottom flask, 03 (300 mg, 0.936 mmol) was dissolved in acetic acid (5 mL), and the mixture was reacted at 70° C. for 2.5 h and concentrated to dryness by rotary evaporation. The residue was purified by column chromatography (dichloromethane:methanol=10:1) to give the target product as a pale yellow solid (250 mg, 88.3% yield). LC-MS: 303.2 [M+H]⁺.

### Step 3

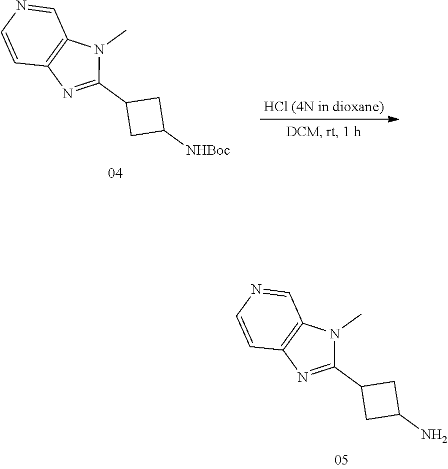 [SMILES: Cn1c(C2CC(NC(=O)OC(C)(C)C)C2)nc2ccncc21 ; Cn1c(C2CC(N)C2)nc2ccncc21]

In a 50 mL single-neck flask, 04 (150 mg, 0.496 mmol) was dissolved in dichloromethane (5 mL), and hydrochloric acid in 1,4-dioxane (0.5 mL) was added. The mixture was stirred at room temperature for 1 h and concentrated by rotary evaporation to remove the solvent to give the target product as a yellow solid (100 mg, 99.7% yield), which could be directly used in the next step without further purification. LC-MS: 203.1 [M+H]⁺.

### Step 4

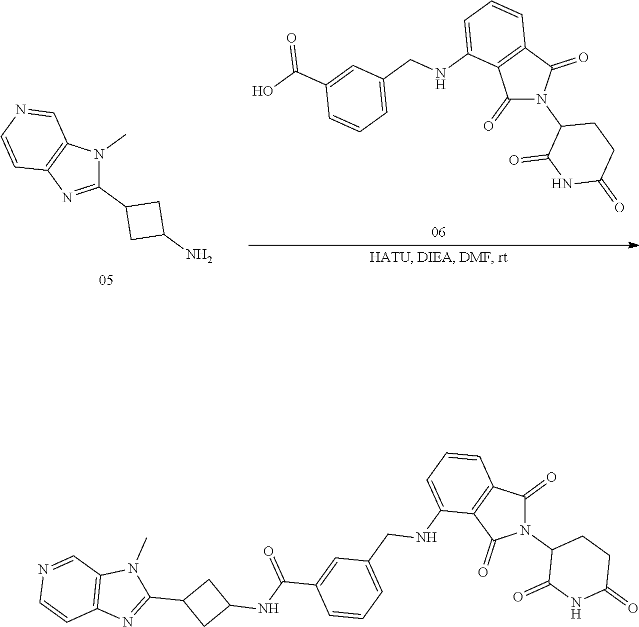 [SMILES: Cn1c(C2CC(N)C2)nc2ccncc21 ; O=C1CCC(N2C(=O)c3cccc(NCc4cccc(C(=O)O)c4)c3C2=O)C(=O)N1 ; Cn1c(C2CC(NC(=O)c3cccc(CNc4cccc5c4C(=O)N(C4CCC(=O)NC4=O)C5=O)c3)C2)nc2ccncc21]

05 (40 mg, 0.198 mmol) was dissolved in N,N-dimethylformamide (5 mL) at room temperature, and 06 (81 mg, 0.199 mmol), HATU (112 mg, 0.294 mmol) and N,N-diisopropylethylamine (77 mg, 0.596 mmol) were added successively. The above mixture was stirred at room temperature for 1 h. The reaction mixture was purified by preparative high performance liquid chromatography to give a yellowish-green solid (65 mg, 55.6% yield). LC-MS: 592.3 [M+H]⁺, H NMR (400 MHz, DMSO-d₆) δ 11.11 (s, 1H), 9.43 (d, J=5.0 Hz, 1H), 8.91-8.80 (m, 1H), 8.60 (dd, J=8.8, 6.6 Hz, 1H), 8.21 (dd, J=31.7, 6.5 Hz, 1H), 7.89 (d, J=4.9 Hz, 1H), 7.77 (d, J=7.9 Hz, 1H), 7.62-7.48 (m, 2H), 7.44 (dt, J=11.4, 7.6 Hz, 1H), 7.30 (d, J=4.3 Hz, 1H), 7.03 (dd, J=7.0, 2.5 Hz, 1H), 6.95 (dd, J=8.6, 5.0 Hz, 1H), 5.08 (dd, J=12.8, 5.2 Hz, 1H), 4.77 (d, J=6.4 Hz, 1H), 4.62 (t, J=5.8 Hz, 2H), 4.04-3.97 (m, 1H), 3.93 (d, J=13.0 Hz, 3H), 3.83-3.71 (m, 1H), 2.95-2.71 (m, 4H), 2.63-2.54 (m, 2H), 2.06 (d, J=13.5 Hz, 1H).

### Example 10

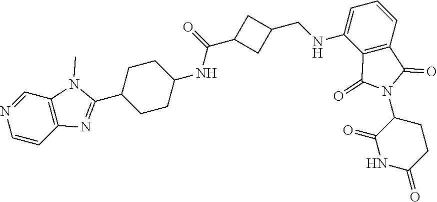 [SMILES: Cn1c(C2CCC(NC(=O)C3CC(CNc4cccc5c4C(=O)N(C4CCC(=O)NC4=O)C5=O)C3)CC2)nc2ccncc21]

### Preparation Steps

### Step 1

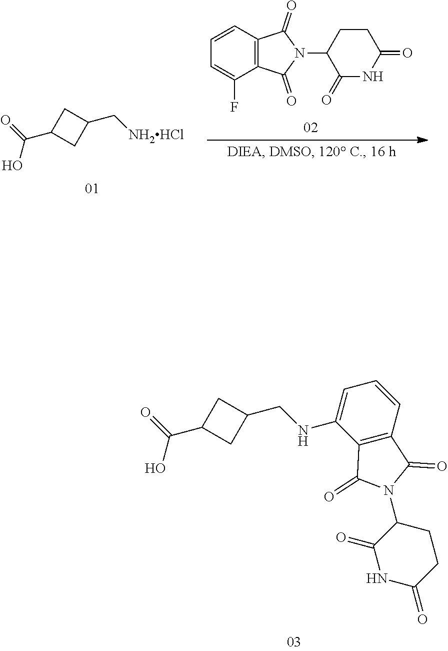

01 (100 mg, 0.604 mmol) was dissolved in dimethyl sulfoxide (1 mL), and 02 (167 mg, 0.604 mmol) and N,N-diisopropylethylamine (234 mg, 1.81 mmol) were added successively. The reaction system was heated to 120° C. and stirred for 16 h in an argon atmosphere. The reaction mixture was purified by preparative high performance liquid chromatography to give a yellowish-green solid (100 mg, 42.9% yield). LC-MS: 386.2 [M+H]⁺.

### Step 2

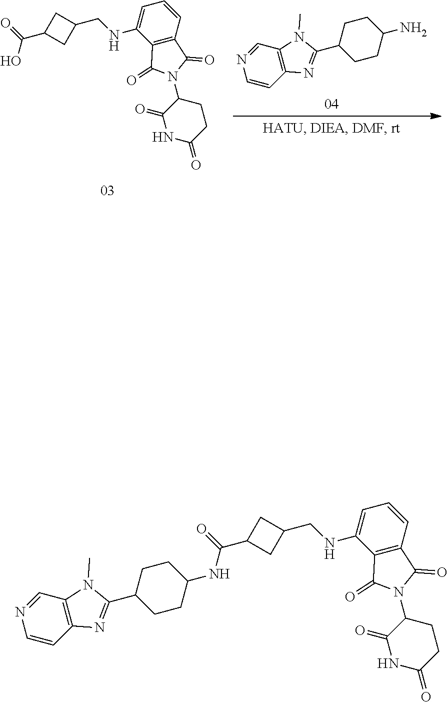

03 (50 mg, 0.13 mmol) was dissolved in N,N-dimethylformamide (1 mL) at room temperature, and 04 (30 mg, 0.13 mmol), HATU (74 mg, 0.195 mmol) and N,N-diisopropylethylamine (51 mg, 0.395 mmol) were added successively. The above mixture was stirred at room temperature for 1 h. The reaction mixture was purified by preparative high performance liquid chromatography to give a yellowish-green solid (45.90 mg, 55.8% yield). LC-MS: 598.3 [M+H]⁺.

¹H NMR (400 MHz, DMSO-d₆) δ 11.09 (s, 1H), 8.86 (s, 1H), 8.27 (d, J=5.3 Hz, 1H), 8.18 (s, 1H), 7.69-7.62 (m, 1H), 7.61-7.52 (m, 2H), 7.13 (t, J=8.2 Hz, 1H), 7.03 (dd, J=7.0, 2.8 Hz, 1H), 6.53 (d, J=25.7 Hz, 1H), 5.05 (dd, J=12.7, 5.2 Hz, 1H), 3.86 (d, J=5.0 Hz, 3H), 3.59 (s, 1H), 3.42-3.37 (m, 1H), 3.05-2.81 (m, 3H), 2.62-2.53 (m, 2H), 2.25-2.11 (m, 2H), 2.01 (dd, J=23.0, 9.7 Hz, 4H), 1.89 (s, 4H), 1.71 (dt, J=24.7, 9.7 Hz, 3H), 1.36 (dd, J=25.8, 14.0 Hz, 2H).

### Example 11

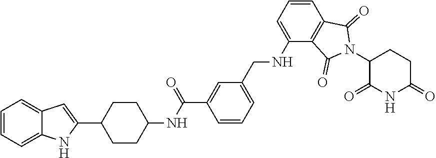 [SMILES: O=C1CCC(N2C(=O)c3cccc(NCc4cccc(C(=O)NC5CCC(c6cc7ccccc7[nH]6)CC5)c4)c3C2=O)C(=O)N1]

### Preparation Steps

### Step 1

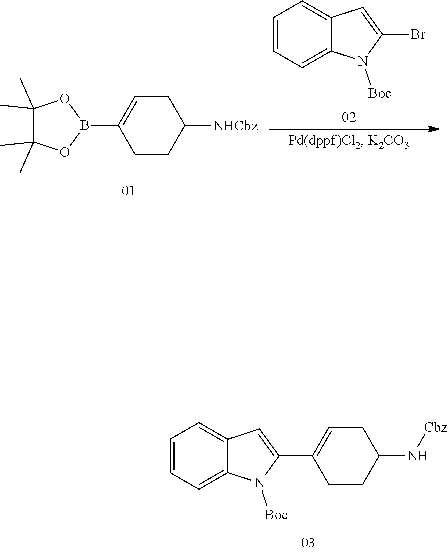 [SMILES: CC1(C)OB(C2=CCC(NC(=O)OCc3ccccc3)CC2)OC1(C)C ; CC(C)(C)OC(=O)n1c(Br)cc2ccccc21 ; CC(C)(C)OC(=O)n1c(C2=CCC(NC(=O)OCc3ccccc3)CC2)cc2ccccc21]

In a 100 mL single-neck flask, 01 (200 mg, 0.56 mmol) was dissolved in dioxane (5 mL), and 02 (165 mg, 0.56 mmol), potassium carbonate (232 mg, 1.68 mmol), Pd(dppf)Cl₂ (41 mg, 0.056 mmol) and water (1.2 mL) were added. The mixture was stirred in a nitrogen atmosphere at 100° C. for 16 h, cooled to room temperature, quenched with water (10 mL), and extracted with ethyl acetate (15 mL×2), and the organic phases were combined, washed with saturated brine (20 mL), dried over anhydrous sodium sulfate, and concentrated to dryness by rotary evaporation to give a crude product, which was then purified by Pre-TLC (PE:EA=20:1) to give the product as a white solid (160 mg, 64% yield). LC-MS: 469.1 [M+Na]⁺, 391.0 [M−56+H]⁺.

### Step 2

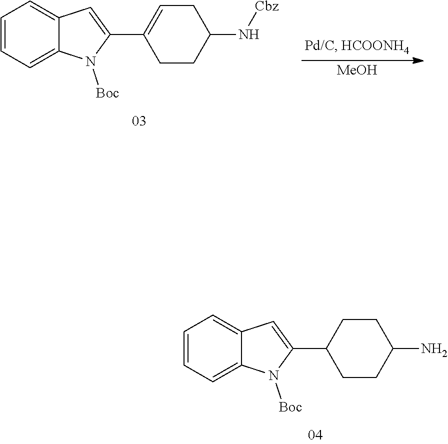

In a 100 mL single-neck flask, 03 (120 mg, 0.27 mmol) was dissolved in methanol (5 mL), and 10% palladium on carbon (10 mg) and ammonium formate (339 mg, 5.38 mmol) were added. The mixture was stirred at 25° C. for 16 h and filtered, and the filtrate was concentrated to dryness by rotary evaporation. The residue was purified by Pre-TLC (DCM:MeOH=12:1) to give the product as a white solid (50 mg, 59.5% yield). LC-MS: 315.3 [M+H]⁺.

### Step 3

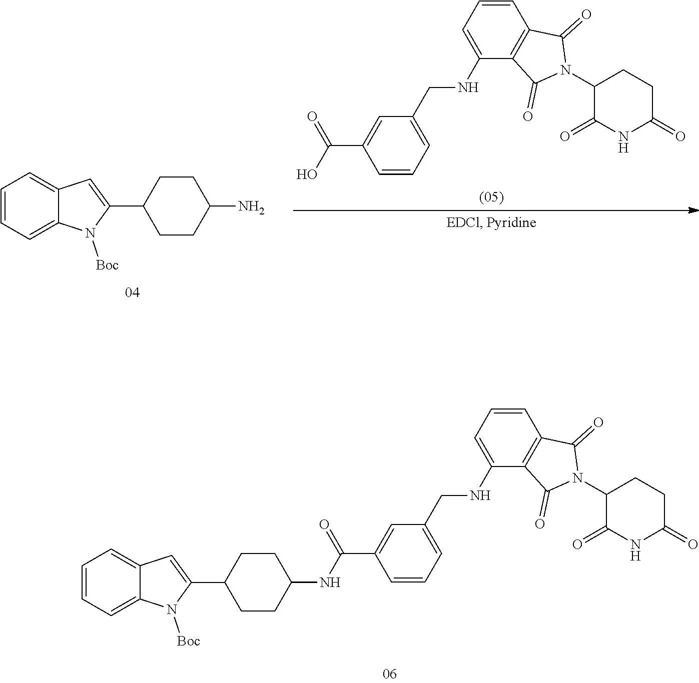 [SMILES: CC(C)(C)OC(=O)n1c(C2CCC(N)CC2)cc2ccccc21 ; O=C1CCC(N2C(=O)c3cccc(NCc4cccc(C(=O)O)c4)c3C2=O)C(=O)N1 ; CC(C)(C)OC(=O)n1c(C2CCC(NC(=O)c3cccc(CNc4cccc5c4C(=O)N(C4CCC(=O)NC4=O)C5=O)c3)CC2)cc2ccccc21]

04 (50 mg, 0.16 mmol) was dissolved in pyridine (2 mL) at room temperature, and 05 (65 mg, 0.16 mmol) and EDCI (92 mg, 0.48 mmol) were added. The mixture was stirred at room temperature for 16 h. The reaction mixture was concentrated, and the residue was purified by Pre-TLC (DCM:MeOH=30:1) to give the target product as a yellow solid (20 mg, 17.9% yield). LC-MS: 726.3 [M+H]⁺.

### Step 4

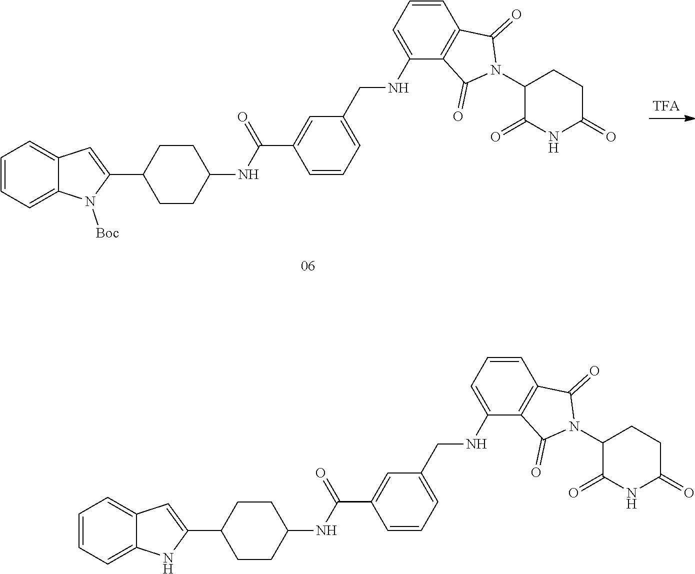 [SMILES: CC(C)(C)OC(=O)n1c(C2CCC(NC(=O)c3cccc(CNc4cccc5c4C(=O)N(C4CCC(=O)NC4=O)C5=O)c3)CC2)cc2ccccc21 ; O=C1CCC(N2C(=O)c3cccc(NCc4cccc(C(=O)NC5CCC(c6cc7ccccc7[nH]6)CC5)c4)c3C2=O)C(=O)N1]

06 (20 mg, 0.028 mmol) was dissolved in DCM (3 mL) at room temperature, and TFA (0.5 mL) was added. The mixture was stirred at room temperature for 2 h. The reaction mixture was concentrated, and the residue was purified by preparative high performance liquid chromatography to give the product as a yellow solid (2.06 mg, 12.2% yield). LC-MS: 604.1 [M+H]⁺, ¹HNMR (400 MHz, DMSO-d₆) δ 11.11 (s, 1H), 10.89-10.86 (m, 1H), 8.30 (d, J=7.5 Hz, 1H), 8.11 (s, 1H), 7.86 (d, J=16.9 Hz, 1H), 7.73 (dd, J=16.1, 7.4 Hz, 1H), 7.51 (dd, J=17.8, 9.6 Hz, 3H), 7.42 (dd, J=17.8, 7.2 Hz, 2H), 7.34-7.25 (m, 2H), 6.99-6.92 (m, 4H), 6.24 (s, 1H), 6.14 (s, 1H), 5.08 (dd, J=12.8, 5.9 Hz, 1H), 4.67-4.57 (m, 3H), 4.03-3.96 (m, 2H), 3.85 (s, 3H), 2.68 (s, 1H), 2.54 (s, 3H), 2.10 (d, J=12.1 Hz, 2H), 1.99 (s, 1H), 1.71 (s, 1H), 1.63-1.50 (m, 2H).

### Example 12

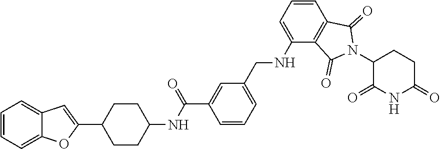 [SMILES: O=C1CCC(N2C(=O)c3cccc(NCc4cccc(C(=O)NC5CCC(c6cc7ccccc7o6)CC5)c4)c3C2=O)C(=O)N1]

### Preparation Steps

### Step 1

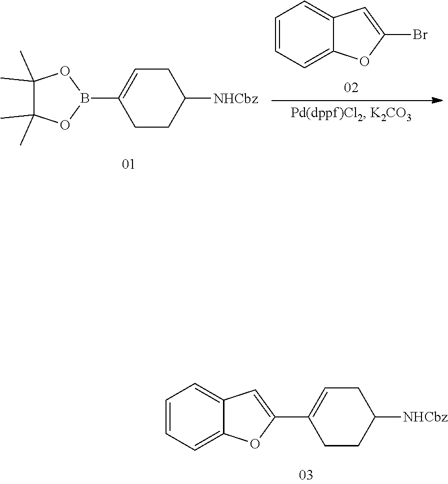

01 (300 mg, 0.840 mmol) was dissolved in dioxane (10 mL) and water (2 mL) at room temperature, and 2-bromo-1-benzofuran (166 mg, 0.840 mmol), potassium carbonate (348 mg, 2.52 mmol) and [1,1′-bis(diphenylphosphino)ferrocene]palladium dichloride (61.5 mg, 0.084 mmol) were added. The above mixture was stirred in an argon atmosphere at 90° C. for 16 h and concentrated to dryness by rotary evaporation, and water (30 mL) was added, followed by extraction with ethyl acetate (20 mL×3). The organic phase was washed with saturated brine (20 mL), dried over anhydrous sodium sulfate, and filtered, and the filtrate was concentrated to dryness by rotary evaporation. The residue was purified by flash silica gel column chromatography (petroleum ether:ethyl acetate=5:1) and preparative thin-layer chromatography (petroleum ether:ethyl acetate=8:1) to give the target product as a white solid (77 mg, 26.4% yield). LC-MS: 348.2 [M+H]⁺.

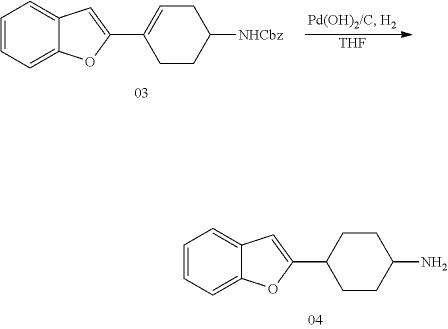 [SMILES: O=C(NC1CC=C(c2cc3ccccc3o2)CC1)OCc1ccccc1 ; NC1CCC(c2cc3ccccc3o2)CC1 ; "THF"]

03 (77 mg, 0.222 mmol) was dissolved in tetrahydrofuran (3 mL) at room temperature, and palladium hydroxide/carbon (20% palladium, 20 mg) was added. The above mixture was stirred in a hydrogen atmosphere at room temperature for 16 h, filtered, and concentrated to dryness by rotary evaporation to give a brownish-yellow oil as the target product (48 mg, crude). LC-MS: 216.1 [M+H]⁺.

### Step 3

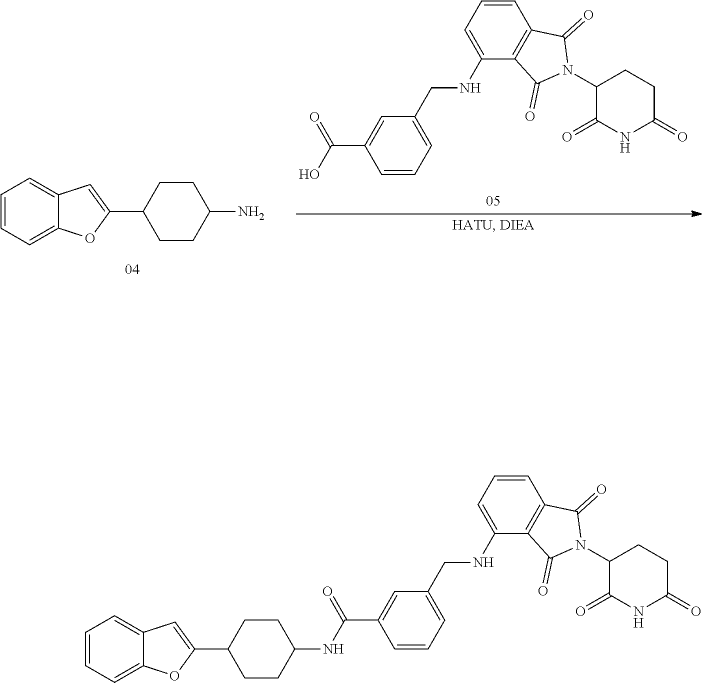 [SMILES: O=C1CCC(N2C(=O)c3cccc(NCc4cccc(C(=O)O)c4)c3C2=O)C(=O)N1 ; NC1CCC(c2cc3ccccc3o2)CC1 ; O=C1CCC(N2C(=O)c3cccc(NCc4cccc(C(=O)NC5CCC(c6cc7ccccc7o6)CC5)c4)c3C2=O)C(=O)N1]

05 (90.8 mg, 0.223 mmol), 04 (48 mg, 0.223 mmol) and N,N-diisopropylethylamine (86.5 mg, 0.669 mmol) were mixed in anhydrous N,N-dimethylformamide (1 mL) at room temperature, and (7-azabenzotriazole)-tetramethyluronium hexafluorophosphate (127 mg, 0.335 mmol) was added with stirring at room temperature. The reaction mixture was stirred at room temperature for 2 h. The mixture was purified by preparative chromatography (C18, 5-100% acetonitrile/water as mobile phase, 0.10% formic acid) to separate the target product as a yellow solid (31.12 mg, 23.1% yield). LC-MS: 605.3 [M+H]⁺. ¹H NMR (400 MHz, DMSO-d₆) δ 11.13-10.96 (m, 1H), 8.35-8.12 (m, 1H), 7.89-7.82 (m, 1H), 7.77-7.67 (m, 1H), 7.61-7.35 (m, 5H), 7.33-7.25 (m, 1H), 7.24-7.15 (m, 2H), 7.12-6.98 (m, 1H), 6.98-6.85 (m, 1H), 6.70-6.56 (m, 1H), 5.12-5.03 (m, 1H), 4.77-4.56 (m, 2H), 3.90-3.79 (m, 1H), 2.96-2.72 (m, 2H), 2.64-2.51 (m, 2H), 2.19-2.09 (m, 2H), 2.07-1.80 (m, 3H), 1.75-1.63 (m, 1H), 1.63-1.46 (m, 3H).

### Example 13

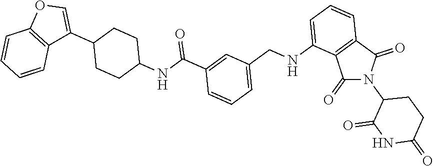 [SMILES: O=C1CCC(N2C(=O)c3cccc(NCc4cccc(C(=O)NC5CCC(c6coc7ccccc67)CC5)c4)c3C2=O)C(=O)N1]

### Preparation Steps

### Step 1

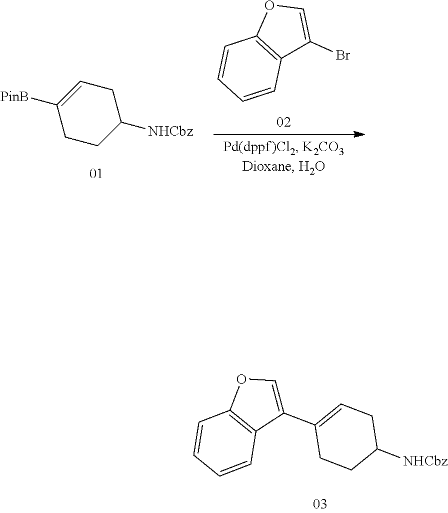

Starting materials 01 (200 mg, 0.56 mmol), 02 (144 mg, 0.73 mmol) and dichloro[1,1′-bis(diphenylphosphino)ferrocene]palladium (41 mg, 0.06 mmol) were dissolved in 1,4-dioxane (4 mL), and a solution of potassium carbonate (194 mg, 1.4 mmol) in water (1 mL) was added. The reaction mixture was stirred in an argon atmosphere at 90° C. for 3 h. Water (50 mL) was added, followed by extraction with ethyl acetate (50 mL×3). The organic phase was washed with saturated brine (50 mL), dried over anhydrous sodium sulfate, and filtered, and the filtrate was concentrated to dryness by rotary evaporation. The residue was passed through a silica gel column (petroleum ether:ethyl acetate=100:0 to 80:20) and a reversed-phase column (C18, water:acetonitrile=100:0 to 35:65, 0.1% formic acid) to separate the target product as a white solid (50 mg, 26.0% yield). LC-MS: 348.2 [M+H]⁺.

### Step 2

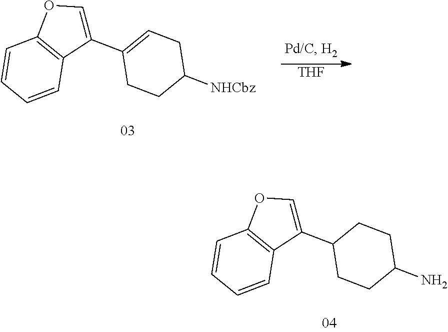 [SMILES: O=C(NC1CC=C(c2coc3ccccc23)CC1)OCc1ccccc1 ; NC1CCC(c2coc3ccccc23)CC1 ; "THF"]

03 (50 mg, 0.14 mmol) was dissolved in tetrahydrofuran (3 mL) at room temperature, and wet palladium on carbon (25 mg) was added. The mixture was purged twice with hydrogen, stirred in a hydrogen atmosphere at room temperature for 16 h, and filtered, and the filtrate was concentrated to dryness by rotary evaporation to give the target product as a white solid (13 mg, 44.0% yield). LC-MS: 216.1 [M+H]⁺.

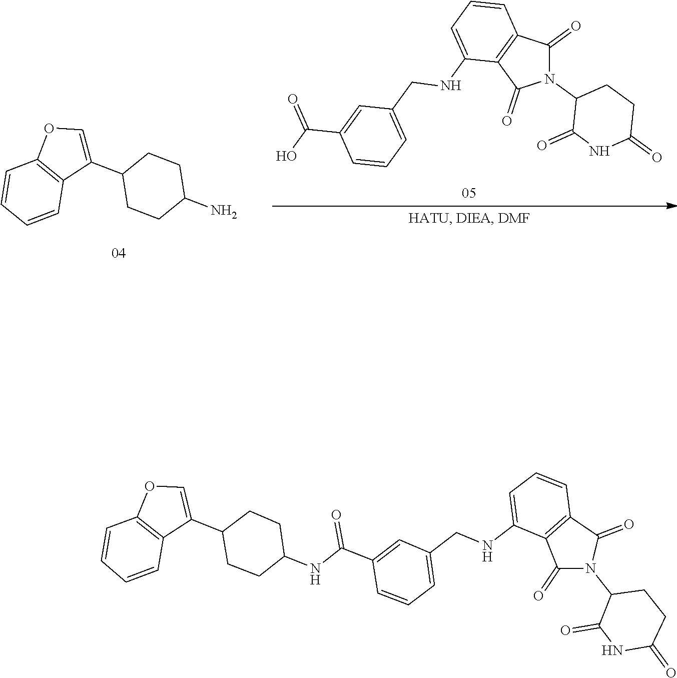 [SMILES: NC1CCC(c2coc3ccccc23)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6coc7ccccc67)CC5)c4)c3C2=O)C(=O)N1]

04 (13 mg, 0.06 mmol), 05 (25 mg, 0.06 mmol) and N,N-diisopropylethylamine (24 mg, 0.18 mmol) were dissolved in N,N-dimethylformamide (0.5 mL) at room temperature, and (7-azabenzotriazole)-tetramethyluronium hexafluorophosphate (28 g, 0.07 mmol) was added with stirring at room temperature. The reaction mixture was stirred at room temperature for 3 h. The mixture was purified by preparative chromatography (C18, 5-100% acetonitrile/water as mobile phase, 0.1% formic acid) to separate the target product as a yellow solid (9.65 mg, 26.6% yield). LC-MS: 605.3 [M+H]⁺. ¹H NMR (400 MHz, DMSO-d₆) δ 11.04 (s, 1H), 8.23 (dd, J=64.4, 7.6 Hz, 1H), 7.85 (d, J=16.9 Hz, 1H), 7.72 (dd, J=18.5, 7.6 Hz, 1H), 7.58-7.45 (m, 4H), 7.44-7.35 (m, 1H), 7.32-7.25 (m, 1H), 7.24-7.16 (m, 2H), 7.02 (t, J=7.7 Hz, 1H), 6.94 (t, 1H), 6.63 (d, J=41.4 Hz, 1H), 5.07 (dt, J=11.1, 5.4 Hz, 1H), 4.60 (dd, J=12.8, 6.0 Hz, 2H), 3.91 (d, J=58.3 Hz, 1H), 2.96-2.84 (m, 1H), 2.81-2.55 (m, 2H), 2.15 (d, J=9.4 Hz, 2H), 2.08-1.78 (m, 4H), 1.74-1.46 (m, 4H).

### Example 14

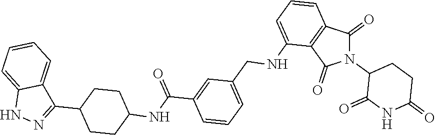 [SMILES: O=C1CCC(N2C(=O)c3cccc(NCc4cccc(C(=O)NC5CCC(c6n[nH]c7ccccc67)CC5)c4)c3C2=O)C(=O)N1]

### Preparation Steps

### Step 1

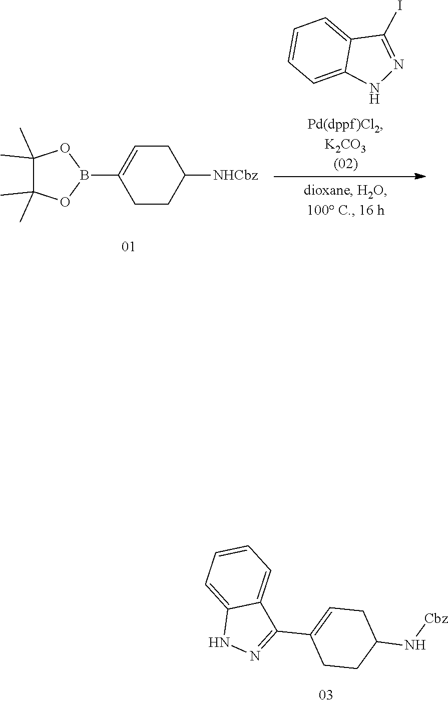 [SMILES: Ic1n[nH]c2ccccc12 ; CC1(C)OB(C2=CCC(NC(=O)OCc3ccccc3)CC2)OC1(C)C ; O=C(NC1CC=C(c2n[nH]c3ccccc23)CC1)OCc1ccccc1]

To a 50 mL single-neck flask were added 01 (431 mg, 1.23 mmol), 02 (300 mg, 1.23 mmol), Pd(dppf)Cl₂ (90 mg, 0.12 mmol), K₂CO₃ (509 mg, 3.69 mmol), 1,4-dioxane (4 mL) and water (1 mL), and the mixture was reacted in a nitrogen atmosphere at 100° C. for 16 h, cooled to room temperature, quenched with water (10 mL), and extracted with ethyl acetate (40 mL×3). The organic phase was washed with saturated brine (20 mL×3) and concentrated to dryness by rotary evaporation to give a crude product, which was then purified by pre-TLC (petroleum ether:ethyl acetate=1:1) to give the target product as a yellow solid (173 mg, 57.7% yield). LC-MS: 348.1 [M+H]⁺.

### Step 2

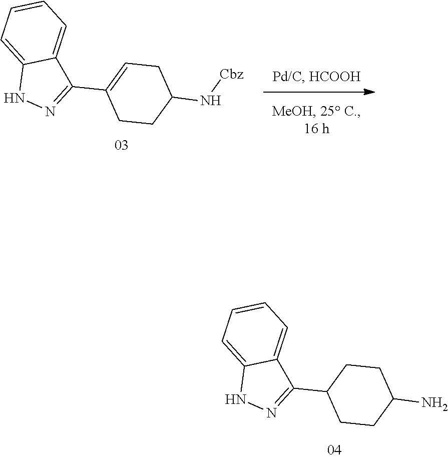 [SMILES: O=C(NC1CC=C(c2n[nH]c3ccccc23)CC1)OCc1ccccc1 ; NC1CCC(c2n[nH]c3ccccc23)CC1]

In a 100 mL single-neck round-bottom flask, 03 (173 mg, 0.50 mmol) and formic acid (2 mL) were dissolved in methanol (3 mL), and palladium on carbon (10 mg) was added. The mixture was reacted in a hydrogen atmosphere at room temperature for 16 h and filtered, and the filtrate was concentrated to dryness by rotary evaporation to give a crude product, which was then purified by pre-TLC (dichloromethane:methanol=5:1) to give the target product as a white solid (86 mg, 41.3% yield). LC-MS: 216.1 [M+H]⁺.

### Step 3

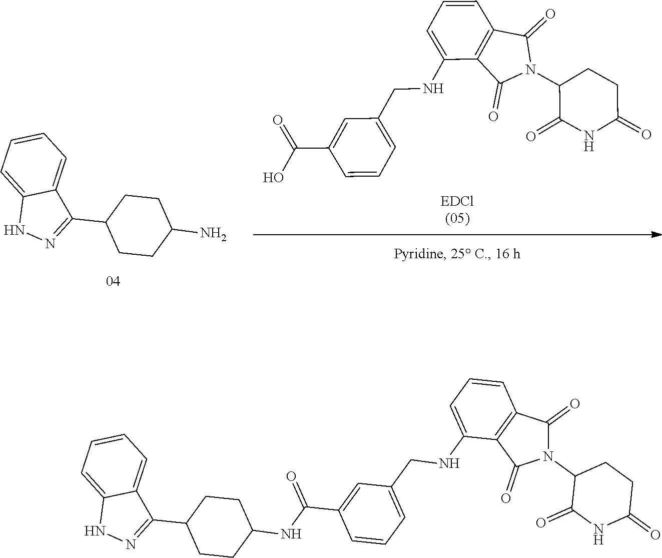 [SMILES: NC1CCC(c2n[nH]c3ccccc23)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6n[nH]c7ccccc67)CC5)c4)c3C2=O)C(=O)N1]

In a 100 mL single-neck flask, 04 (80 mg, 0.37 mmol) was dissolved in pyridine (2 mL), and 05 (151 mg, 0.37 mmol) and EDCI (212 mg, 1.11 mmol) were added. The mixture was stirred at room temperature for 16 h and purified by preparative reversed-phase chromatography to give target product 1 as a yellow solid (1.04 mg, 1.3% yield). LC-MS: 605.1 [M+H]⁺, ¹H NMR (400 MHz, DMSO-d₆) δ 12.61 (s, 1H), 11.10 (s, 1H), 8.31 (d, J=8.0 Hz, 1H), 7.88 (s, 1H), 7.83 (d, J=7.8 Hz, 1H), 7.75 (d, J=8.0 Hz, 1H), 7.51 (d, J=7.5 Hz, 2H), 7.44 (t, J=8.2 Hz, 2H), 7.30 (s, 2H), 7.04 (d, J=7.1 Hz, 2H), 6.97 (d, J=9.0 Hz, 1H), 5.08 (d, J=8.1 Hz, 1H), 4.62 (d, J=5.5 Hz, 2H), 3.91 (s, 1H), 3.02 (s, 1H), 2.89 (s, 1H), 2.67 (s, 1H), 2.06-1.98 (m, 6H), 1.79 (d, J=13.0 Hz, 2H), 1.66-1.53 (m, 3H).

### Example 15

The mixture obtained in step 3 of Example 14 was purified by preparative reversed-phase chromatography to give target product 2 as a yellow solid (2.20 mg, 2.8% yield). LC-MS: 605.1 [M+H]⁺. ¹H NMR (400 MHz, DMSO-d₆) δ 12.62 (s, 1H), 11.09 (s, 1H), 8.20 (d, J=6.8 Hz, 1H), 7.86 (s, 1H), 7.79 (d, J=8.3 Hz, 1H), 7.73 (d, J=7.4 Hz, 1H), 7.53-7.44 (m, 3H), 7.39 (t, J=7.6 Hz, 1H), 7.32-7.26 (m, 2H), 7.04 (dd, J=17.5, 7.2 Hz, 2H), 6.95 (d, J=8.7 Hz, 1H), 5.07 (dd, J=12.7, 5.2 Hz, 1H), 4.60 (d, J=5.8 Hz, 2H), 4.02 (s, 1H), 2.87 (d, J=13.3 Hz, 1H), 2.61 (s, 2H), 2.24 (s, 2H), 2.06 (s, 1H), 1.88 (s, 4H), 1.71 (s, 2H), 1.24 (s, 1H).

### Example 16

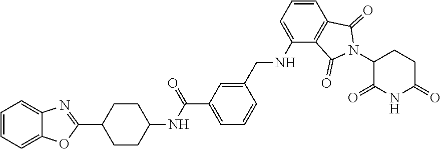 [SMILES: O=C1CCC(N2C(=O)c3cccc(NCc4cccc(C(=O)NC5CCC(c6nc7ccccc7o6)CC5)c4)c3C2=O)C(=O)N1]

### Preparation Steps

### Step 1

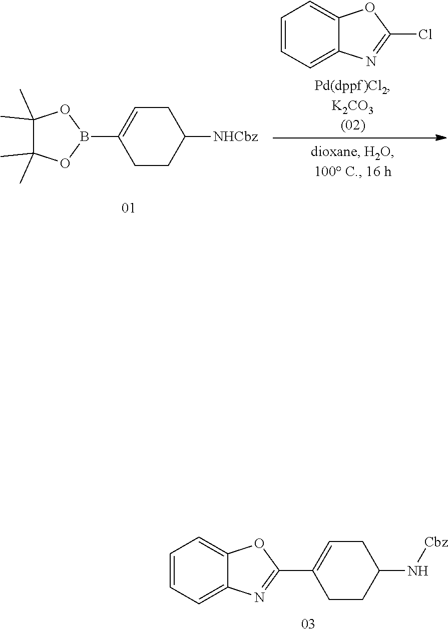 [SMILES: Clc1nc2ccccc2o1 ; CC1(C)OB(C2=CCC(NC(=O)OCc3ccccc3)CC2)OC1(C)C ; O=C(NC1CC=C(c2nc3ccccc3o2)CC1)OCc1ccccc1]

To a 50 mL single-neck flask were added 01 (227 mg, 0.64 mmol), 02 (100 mg, 0.64 mmol), Pd(dppf)Cl₂ (45.4 mg, 0.06 mmol), K₂CO₃ (263 mg, 1.90 mmol), 1,4-dioxane (2 mL) and water (1 mL), and the mixture was reacted in a nitrogen atmosphere at 100° C. for 16 h, cooled to room temperature, quenched with water (10 mL), and extracted with ethyl acetate (40 mL×3). The organic phase was washed with saturated brine (20 mL×3) and concentrated to dryness by rotary evaporation, and the residue was purified by pre-TLC (petroleum ether:ethyl acetate=5:1) to give the target product as a yellow solid (116 mg, 51.1% yield). LC-MS: 349.0 [M+H]⁺.

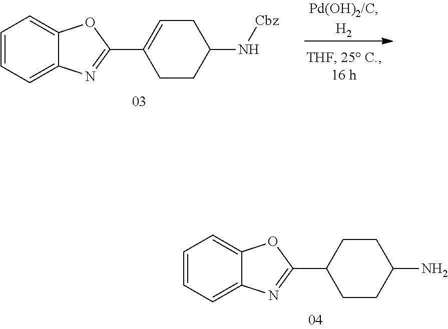 [SMILES: O=C(NC1CC=C(c2nc3ccccc3o2)CC1)OCc1ccccc1 ; NC1CCC(c2nc3ccccc3o2)CC1]

In a 100 mL single-neck round-bottom flask, 03 (116 mg, 0.33 mmol) was dissolved in tetrahydrofuran (3 mL), and palladium hydroxide/carbon (10 mg) was added. The mixture was reacted in a hydrogen atmosphere at room temperature for 16 h and filtered, and the filtrate was concentrated to dryness by rotary evaporation to give a crude product, which was then purified by pre-TLC (petroleum ether:ethyl acetate=3:1) to give the target product as a white solid (48 mg, 41.3% yield). LC-MS: 217.2 [M+H]⁺.

### Step 3

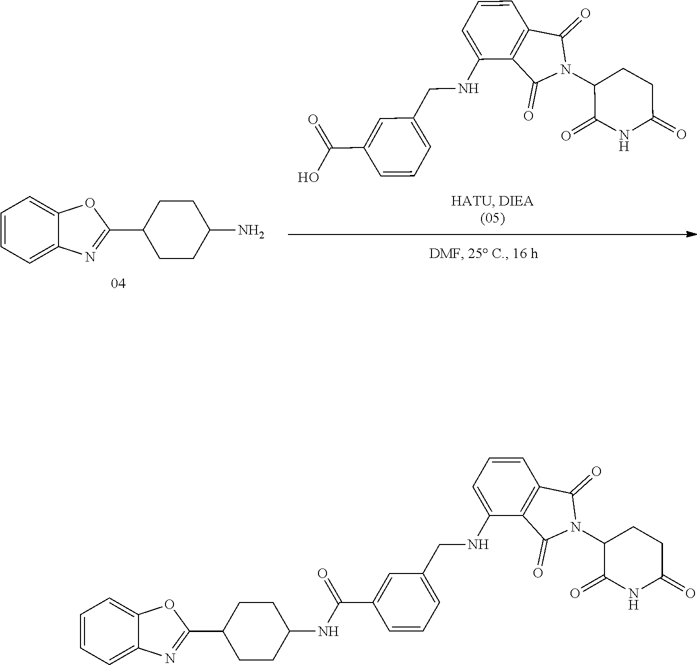 [SMILES: O=C1CCC(N2C(=O)c3cccc(NCc4cccc(C(=O)O)c4)c3C2=O)C(=O)N1 ; NC1CCC(c2nc3ccccc3o2)CC1 ; O=C1CCC(N2C(=O)c3cccc(NCc4cccc(C(=O)NC5CCC(c6nc7ccccc7o6)CC5)c4)c3C2=O)C(=O)N1]

In a 100 mL single-neck flask, 04 (48 mg, 0.14 mmol) was dissolved in N,N-dimethylformamide (2 mL), and HATU (126 mg, 0.21 mmol), DIEA (85 mg, 0.42 mmol) and 05 (48 mg, 0.14 mmol) were added. The mixture was stirred at room temperature for 16 h and purified by preparative reversed-phase chromatography to give target product 1 as a yellow solid (5.25 mg, 11.0% yield). LC-MS: 606.0 [M+H]⁺, ¹H NMR (400 MHz, CD₃OD) δ 7.86 (s, 1H), 7.72 (d, J=7.7 Hz, 1H), 7.67-7.62 (m, 1H), 7.60-7.54 (m, 2H), 7.51-7.41 (m, 2H), 7.39-7.33 (m, 2H), 7.06 (d, J=7.1 Hz, 1H), 6.94 (d, J=8.5 Hz, 1H), 5.08 (dd, J=12.4, 5.5 Hz, 1H), 4.63 (s, 2H), 3.98 (s, 1H), 3.01 (s, 1H), 2.90-2.81 (m, 1H), 2.80-2.69 (m, 2H), 2.31 (d, J=12.2 Hz, 2H), 2.19-2.09 (m, 3H), 1.90-1.75 (m, 2H), 1.59-1.50 (m, 2H).

### Example 17

The mixture obtained in step 3 of Example 16 was purified by preparative reversed-phase chromatography to give target product 2 as a yellow solid (2.16 mg, 4.5% yield). LC-MS: 606.1 [M+H]⁺. ¹H NMR (400 MHz, CD₃OD) δ 7.81 (s, 1H), 7.66 (dd, J=11.3, 5.5 Hz, 2H), 7.60-7.56 (m, 1H), 7.52 (d, J=7.9 Hz, 1H), 7.45 (d, J=7.9 Hz, 1H), 7.43-7.39 (m, 1H), 7.37-7.33 (m, 2H), 7.03 (d, J=7.1 Hz, 1H), 6.92 (d, J=8.5 Hz, 1H), 5.07-5.02 (m, 1H), 4.59 (s, 2H), 4.56 (s, 2H), 4.07 (d, J=4.6 Hz, 1H), 2.83 (dd, J=18.2, 4.8 Hz, 1H), 2.75-2.60 (m, 2H), 2.40 (d, J=9.5 Hz, 2H), 2.03 (dd, J=9.1, 4.9 Hz, 3H), 1.89 (s, 2H), 1.83-1.73 (m, 2H).

### Example 18

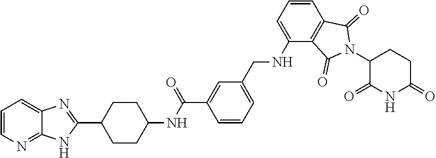 [SMILES: O=C1CCC(N2C(=O)c3cccc(NCc4cccc(C(=O)NC5CCC(c6nc7cccnc7[nH]6)CC5)c4)c3C2=O)C(=O)N1]

### Preparation Steps

### Step 1

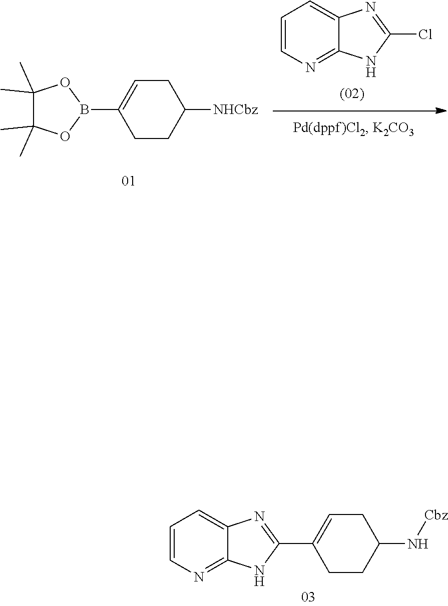 [SMILES: CC1(C)OB(C2=CCC(NC(=O)OCc3ccccc3)CC2)OC1(C)C ; Clc1nc2cccnc2[nH]1 ; O=C(NC1CC=C(c2nc3cccnc3[nH]2)CC1)OCc1ccccc1]

To a 100 mL single-neck flask were added 01 (150 mg, 0.42 mmol), 02 (64 mg, 0.42 mmol), potassium carbonate (173 mg, 1.26 mmol), Pd(dtbpf)Cl₂ (27 mg, 0.028 mmol), dioxane (4 mL) and water (1 mL), and the mixture was stirred in a nitrogen atmosphere at 100° C. for 16 h, cooled to room temperature, and concentrated to dryness by rotary evaporation to give a crude product, which was then purified by Pre-TLC (PE:EA=1:3) to give the target product as a brown solid (65 mg, 44.4% yield). LC-MS: 349.0 [M+H]⁺.

### Step 2

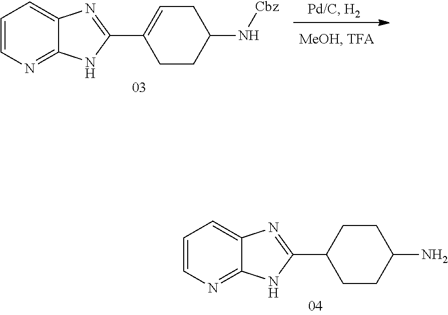 [SMILES: O=C(NC1CC=C(c2nc3cccnc3[nH]2)CC1)OCc1ccccc1 ; NC1CCC(c2nc3cccnc3[nH]2)CC1]

In a 100 mL single-neck flask, 03 (55 mg, 0.16 mmol) was dissolved in methanol (3 mL), and trifluoroacetic acid (1 mL) and palladium on carbon (10 mg) were added. The mixture was stirred in a hydrogen atmosphere for 16 h and filtered, and the filtrate was concentrated to dryness by rotary evaporation to give the target product as a yellow oil, which was directly used in the next step. (36 mg, 100% yield). LC-MS: 217.2 [M+H]⁺.

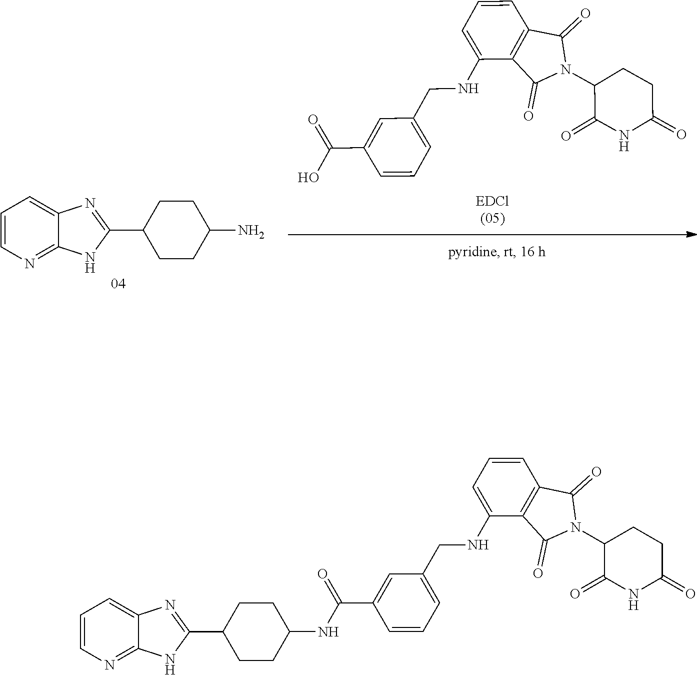 [SMILES: O=C1CCC(N2C(=O)c3cccc(NCc4cccc(C(=O)O)c4)c3C2=O)C(=O)N1 ; NC1CCC(c2nc3cccnc3[nH]2)CC1 ; O=C1CCC(N2C(=O)c3cccc(NCc4cccc(C(=O)NC5CCC(c6nc7cccnc7[nH]6)CC5)c4)c3C2=O)C(=O)N1]

04 (36 mg, 0.166 mmol) was dissolved in pyridine (1 mL) at room temperature, and 05 (67 mg, 0.166 mmol) and EDCI (96 mg, 0.499 mmol) were added. The above mixture was stirred at room temperature for 16 h and concentrated to give a crude product, which was then purified by preparative high performance liquid chromatography to give the target product as a yellow solid (4.93 mg, 4.9% yield). LC-MS: 606.1 [M+H]⁺, ¹H NMR (400 MHz, DMSO-d₆) δ 12.79 (s, 1H), 12.44 (s, 1H), 11.10 (s, 1H), 8.34-8.08 (m, 2H), 7.88 (s, 1H), 7.84 (s, 1H), 7.72 (dd, J=14.8, 7.7 Hz, 1H), 7.54-7.42 (m, 2H), 7.38 (dd, J=15.4, 7.6 Hz, 1H), 7.28 (dd, J=12.5, 6.4 Hz, 1H), 7.16 (s, 1H), 7.02 (dd, J=9.6, 7.1 Hz, 1H), 6.94 (dd, J=11.9, 8.7 Hz, 1H), 5.07 (dd, J=12.8, 5.5 Hz, 1H), 4.60 (dd, J=16.3, 6.3 Hz, 2H), 4.05-3.81 (m, 1H), 3.13 (s, 1H), 2.87 (d, J=12.4 Hz, 1H), 2.69-2.55 (m, 2H), 2.33 (s, 1H), 2.12 (s, 1H), 2.03 (d, J=7.1 Hz, 2H), 1.86 (s, 1H), 1.71 (s, 3H), 1.54 (s, 1H).

### Example 19

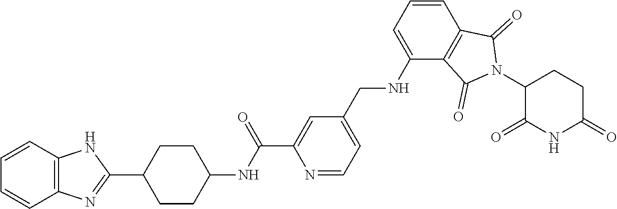 [SMILES: O=C1CCC(N2C(=O)c3cccc(NCc4ccnc(C(=O)NC5CCC(c6nc7ccccc7[nH]6)CC5)c4)c3C2=O)C(=O)N1]

### Preparation Steps

### Step 1

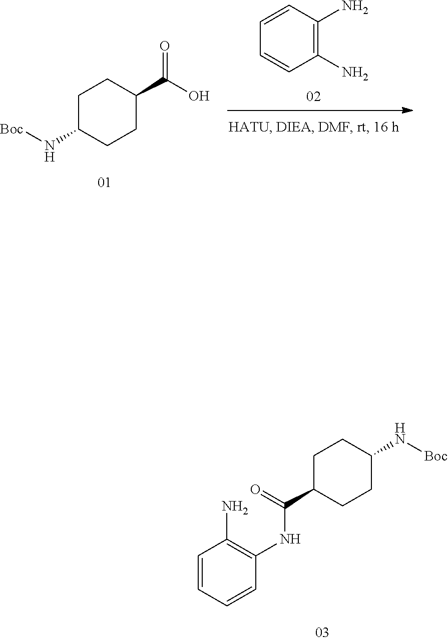 [SMILES: CC(C)(C)OC(=O)N[C@H]1CC[C@H](C(=O)O)CC1 ; Nc1ccccc1N ; CC(C)(C)OC(=O)N[C@H]1CC[C@H](C(=O)Nc2ccccc2N)CC1]

01 (1 g, 4.11 mmol) was dissolved in N,N-dimethylformamide (10 mL) at room temperature, and HATU (3.12 g, 8.22 mmol) was added. After the mixture was stirred at room temperature for 30 min, 02 (444 mg, 4.11 mmol) and DIEA (1.59 g, 12.33 mmol) were added. The reaction mixture was stirred at room temperature for 16 h, diluted with 50 mL of water, and extracted with ethyl acetate (50 mL×3), and the organic phases were washed with saturated brine (50 mL×3), combined, dried over anhydrous sodium sulfate, and concentrated to give a crude product, which was then purified by column chromatography (dichloromethane:methanol=15:1) to give a yellowish-white solid (140 mg, 10.2% yield). LC-MS: 333.4 [M+H]⁺.

### Step 2

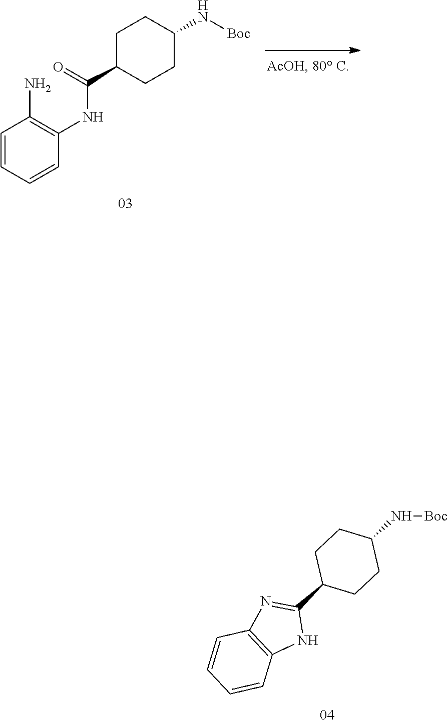 [SMILES: CC(C)(C)OC(=O)N[C@H]1CC[C@H](C(=O)Nc2ccccc2N)CC1 ; CC(C)(C)OC(=O)N[C@H]1CC[C@H](c2nc3ccccc3[nH]2)CC1]

03 (80 mg, 0.24 mmol) was dissolved in acetic acid (5 mL) at room temperature, and the mixture was heated to 80° C. and stirred for 3 h in a nitrogen atmosphere. After being cooled to room temperature, the reaction mixture was diluted with 10 mL of water and extracted with ethyl acetate (10 mL of ×3), and the organic phases were washed with saturated brine (10 mL of ×3), combined, dried over anhydrous sodium sulfate, and concentrated to give a yellowish-brown solid (140 mg, crude). LC-MS: 315.4 [M+H]⁺.

### Step 3

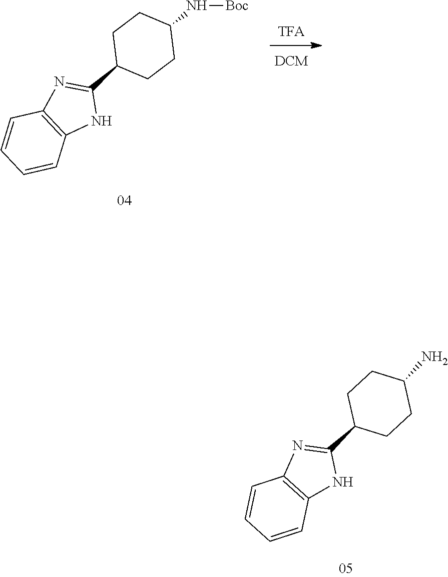 [SMILES: CC(C)(C)OC(=O)N[C@H]1CC[C@H](c2nc3ccccc3[nH]2)CC1 ; N[C@H]1CC[C@H](c2nc3ccccc3[nH]2)CC1]

To a 50 mL single-neck flask were added 04 (100 mg, 0.32 mmol), dichloromethane (6 mL) and trifluoroacetic acid (3 mL), and the mixture was stirred in a nitrogen atmosphere at room temperature for 1 h and concentrated to dryness by rotary evaporation to give a yellowish-brown solid (75 mg, crude).

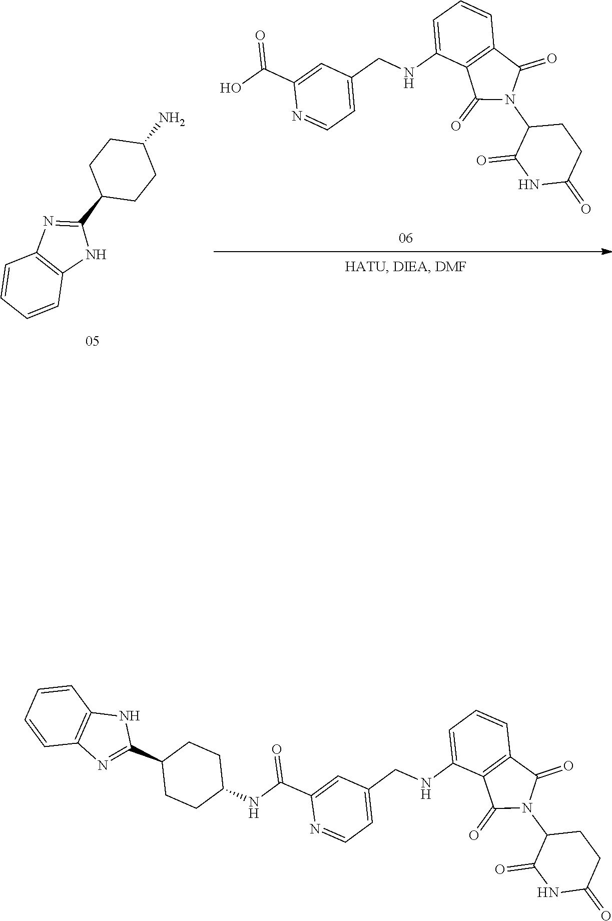 [SMILES: N[C@H]1CC[C@H](c2nc3ccccc3[nH]2)CC1 ; O=C1CCC(N2C(=O)c3cccc(NCc4ccnc(C(=O)O)c4)c3C2=O)C(=O)N1 ; O=C1CCC(N2C(=O)c3cccc(NCc4ccnc(C(=O)N[C@H]5CC[C@H](c6nc7ccccc7[nH]6)CC5)c4)c3C2=O)C(=O)N1]

In a 50 mL single-neck flask, 05 (75 mg, 0.35 mmol) was dissolved in N,N-dimethylformamide (2 mL) at room temperature, and N,N-diisopropylethylamine (136 mg, 1.05 mmol) was added. The mixture was stirred at room temperature for 30 min. Meanwhile, in another 50 mL single-neck flask, 06 (143 mg, 0.35 mmol) was dissolved in N,N-dimethylformamide (2 mL), and HATU (266 mg, 0.70 mmol) was added. The mixture was stirred at room temperature for 30 min. The mixtures in the two flasks were mixed and reacted at room temperature for 16 h. The reaction mixture was purified by preparative chromatography to give the product as a white solid (22.03 mg, 10.4% yield). LC-MS: 605.6 [M+H]⁺, ¹H NMR (400 MHz, DMSO-d6) δ 11.11 (s, 1H), 8.59-8.55 (m, 2H), 8.27 (s, 1H), 8.05 (s, 1H), 7.63 (dd, J=5.8, 3.2 Hz, 2H), 7.57 (d, J=4.8 Hz, 1H), 7.46 (dd, J=15.1, 7.3 Hz, 2H), 7.33 (dd, J=5.8, 3.1 Hz, 2H), 7.05 (d, J=7.0 Hz, 1H), 6.86 (d, J=8.5 Hz, 1H), 5.09 (dd, J=12.6, 5.2 Hz, 1H), 4.71 (d, J=6.3 Hz, 2H), 3.90 (d, J=8.3 Hz, 2H), 2.64-2.54 (m, 3H), 2.18 (d, J=11.8 Hz, 2H), 2.08 (d, J=11.1 Hz, 1H), 2.00 (d, J=10.5 Hz, 2H), 1.83-1.74 (m, 2H), 1.66 (d, J=12.0 Hz, 2H).

### Example 20

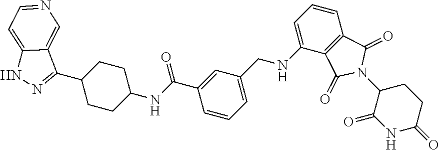 [SMILES: O=C1CCC(N2C(=O)c3cccc(NCc4cccc(C(=O)NC5CCC(c6n[nH]c7ccncc67)CC5)c4)c3C2=O)C(=O)N1]

### Preparation Steps

### Step 1

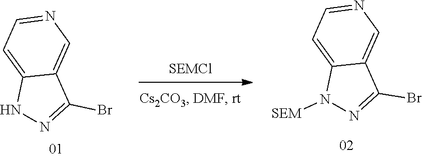 [SMILES: Brc1n[nH]c2ccncc12 ; "SEMCl" ; C[Si](C)(C)CCOCn1nc(Br)c2cnccc21]

01 (500 mg, 2.54 mmol), 2-(trimethylsilyl)ethoxymethyl chloride (0.4 mL) and cesium carbonate (820 mg, 2.54 mmol) were dissolved in N,N-dimethylformamide (10 mL) at room temperature, and the reaction mixture was stirred at room temperature for 16 h. Water (100 mL) was added, followed by extraction with ethyl acetate (100 mL×3). The organic phase was washed with saturated brine (50 mL×3), dried over anhydrous sodium sulfate, and filtered, and the filtrate was concentrated to dryness by rotary evaporation to give the target product as a yellow solid (360 mg, 43.4% yield). LC-MS: 328.1 [M+H]⁺.

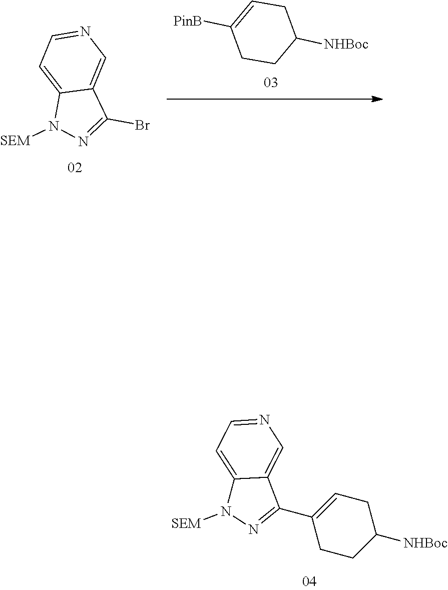

02 (360 mg, 1.10 mmol), 03 (323 mg, 1.65 mmol), [1,1′-bis(diphenylphosphino)ferrocene]palladium dichloride complexed with dichloromethane (161 mg, 0.22 mmol) and potassium carbonate (303 mg, 2.20 mmol) were dissolved in 1,4-dioxane (4 mL) and water (1 mL) at room temperature, and the reaction mixture was stirred at 90° C. for 16 h. Water (100 mL) was added, followed by extraction with ethyl acetate (100 mL×3). The organic phase was washed with saturated brine (100 mL×3), dried over anhydrous sodium sulfate, and filtered, and the filtrate was concentrated to dryness by rotary evaporation to give the target product as a yellow solid (260 mg, 53.3% yield). LC-MS: 445.2 [M+H]⁺.

### Step 3

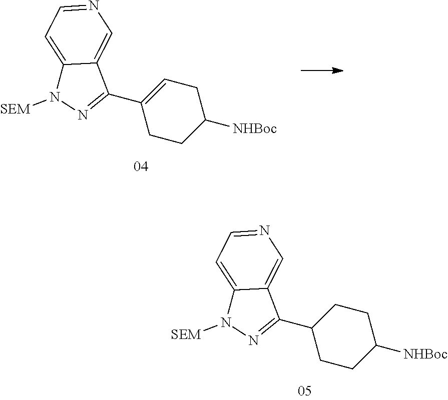 [SMILES: CC(C)(C)OC(=O)NC1CC=C(c2nn(COCC[Si](C)(C)C)c3ccncc23)CC1 ; CC(C)(C)OC(=O)NC1CCC(c2nn(COCC[Si](C)(C)C)c3ccncc23)CC1]

03 (240 mg, 0.54 mmol) was dissolved in tetrahydrofuran (5 mL) at room temperature, and wet palladium on carbon (100 mg) was added. The mixture was purged twice with hydrogen, stirred in a hydrogen atmosphere at room temperature for 16 h, and filtered, and the filtrate was concentrated to dryness by rotary evaporation to give the target product as a yellow solid (100 mg, crude). LC-MS: 447.2 [M+H]⁺.

### Step 4

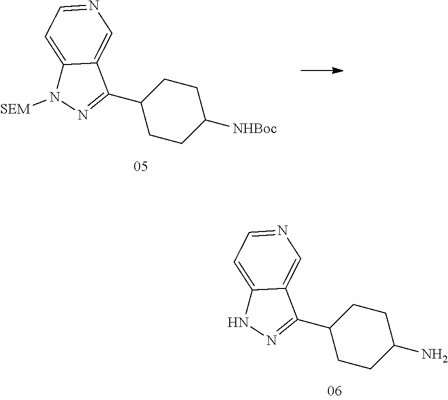 [SMILES: CC(C)(C)OC(=O)NC1CCC(c2nn(COCC[Si](C)(C)C)c3ccncc23)CC1 ; NC1CCC(c2n[nH]c3ccncc23)CC1]

05 (80 mg, 0.18 mmol) was dissolved in dichloromethane (3 mL) at room temperature, and trifluoroacetic acid (1 mL) was added dropwise with stirring at room temperature. The reaction mixture was stirred at room temperature for 6 h and concentrated by rotary evaporation to remove the solvent to give the target product as a yellow oil (80 mg, crude). LC-MS: 217.2 [M+H]⁺.

### Step 5

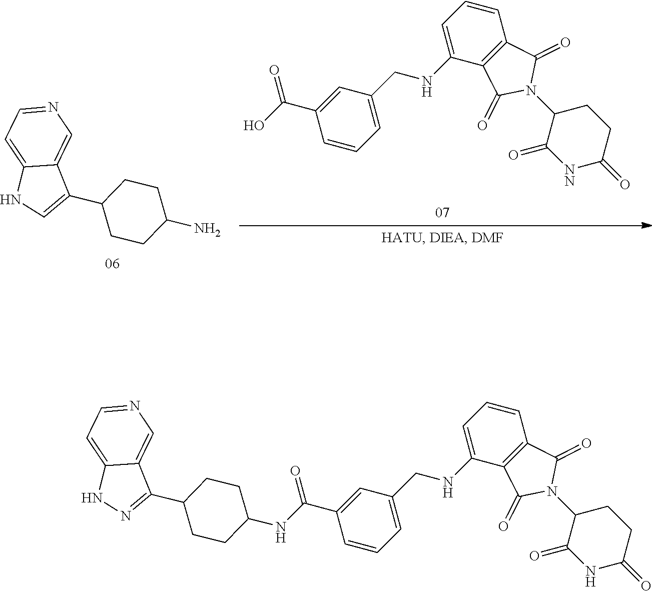

05 (70 mg, 0.32 mmol), 06 (146 mg, 0.38 mmol), N,N-diisopropylethylamine (82 mg, 0.64 mmol) and 2-(7-azobenzotriazol)-N,N,N,N-tetramethyluronium hexafluorophosphate (243 mg, 0.64 mmol) were dissolved in N,N-dimethylformamide (2 mL) at room temperature, and the reaction mixture was stirred at room temperature for 16 h and purified by preparative chromatography (C18, 5-100% acetonitrile/water as mobile phase, 0.1% formic acid) to separate the target product as a yellow solid (29.87 mg, 15.3% yield). LC-MS: 606.2 [M+H]⁺. ¹H NMR (400 MHz, DMSO) δ 11.28-10.88 (m, 1H), 9.76-9.33 (m, 1H), 8.80-8.43 (m, 1H), 8.36-8.14 (m, 1H), 7.97-7.63 (m, 3H), 7.57-7.36 (m, 3H), 7.34-7.23 (m, 1H), 7.07-6.90 (m, 2H), 5.14-4.99 (m, 1H), 4.71-4.53 (m, 2H), 3.30-3.29 (m, 1H), 2.96-2.82 (m, 3H), 2.56 (d, J=8.6 Hz, 1H), 2.28-1.97 (m, 5H), 1.93-1.79 (m, 3H), 1.68-1.54 (m, 1H).

### Example 21

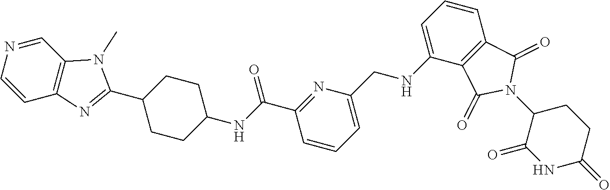 [SMILES: Cn1c(C2CCC(NC(=O)c3cccc(CNc4cccc5c4C(=O)N(C4CCC(=O)NC4=O)C5=O)n3)CC2)nc2ccncc21]

### Preparation Steps

### Step 1

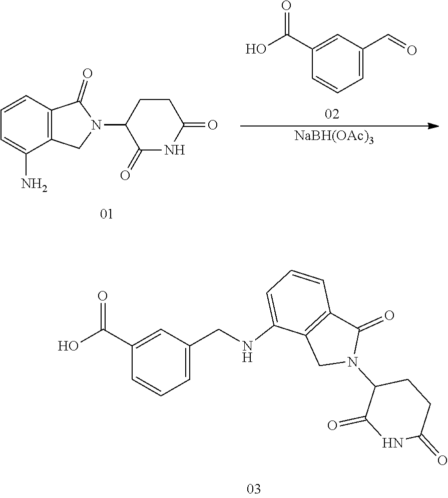 [SMILES: Nc1cccc2c1CN(C1CCC(=O)NC1=O)C2=O ; O=Cc1cccc(C(=O)O)c1 ; O=C1CCC(N2Cc3c(NCc4cccc(C(=O)O)c4)cccc3C2=O)C(=O)N1 ; "NaBH(OAc)3"]

In a 100 mL single-neck flask, 01 (200 mg, 0.771 mmol) was dissolved in acetic acid (2 mL), and the mixture was stirred and warmed to 70° C. After complete dissolution was achieved in the reaction system, dichloromethane (5 mL) and 02 (232 mg, 1.54 mmol) were slowly added, and the mixture was reacted at room temperature for 0.5 h. Sodium triacetoxyborohydride (490 mg, 2.31 mmol) was added, and the mixture was stirred at room temperature for another 16 h, quenched with water (15 mL), and extracted with dichloromethane (20 mL×3). The organic phases were combined, washed with saturated brine (15 mL×3), dried over anhydrous sodium sulfate, and filtered, and the filtrate was concentrated. The residue was purified by column chromatography (dichloromethane:methanol=10:1) to give the target product as a yellow solid (35 mg, 11.5% yield). LC-MS: 394.3 [M+H]⁺.

### Step 2

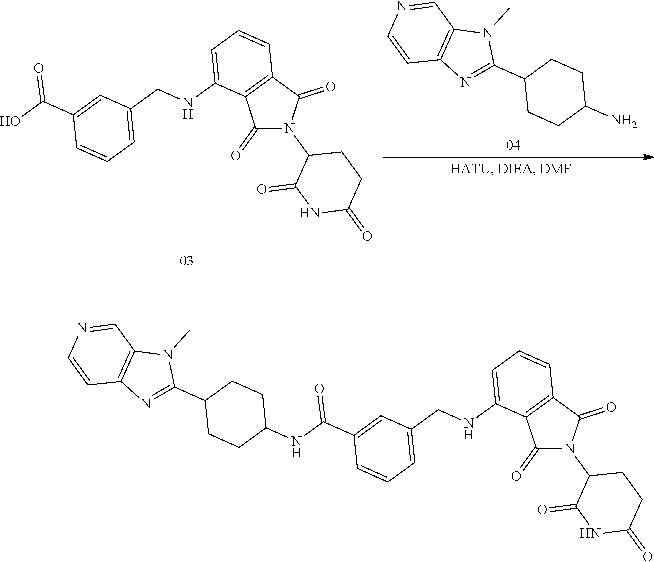 [SMILES: O=C1CCC(N2C(=O)c3cccc(NCc4cccc(C(=O)O)c4)c3C2=O)C(=O)N1 ; Cn1c(C2CCC(N)CC2)nc2ccncc21 ; Cn1c(C2CCC(NC(=O)c3cccc(CNc4cccc5c4C(=O)N(C4CCC(=O)NC4=O)C5=O)c3)CC2)nc2ccncc21]

03 (35 mg, 0.0890 mmol) was dissolved in N,N-dimethylformamide (0.5 mL) at room temperature, and HATU (51 mg, 0.133 mmol), N,N-diisopropylethylamine (34 mg, 0.267 mmol) and 04 (24 mg, 0.0890 mmol) were added successively. The above mixture was stirred at room temperature for 2 h. The reaction mixture was purified by preparative high performance liquid chromatography (FA) to give the target product as a white solid (8.34 mg, 15.5% yield). LC-MS: 606.2 [M+H]⁺.

¹H NMR (400 MHz, DMSO-d₆) δ 11.01 (s, 1H), 8.87 (s, 1H), 8.35-8.20 (m, 2H), 7.89 (s, 1H), 7.72 (d, J=7.2 Hz, 1H), 7.54 (dd, J=12.4, 6.5 Hz, 2H), 7.40 (t, J=7.9 Hz, 1H), 7.21 (t, J=7.8 Hz, 1H), 6.94 (d, J=7.2 Hz, 1H), 6.65 (d, J=8.2 Hz, 1H), 6.41 (d, J=5.9 Hz, 1H), 5.22-4.97 (m, 1H), 4.44 (s, 2H), 4.27 (dd, J=44.7, 17.1 Hz, 2H), 3.88 (d, J=7.6 Hz, 3H), 3.02-2.78 (m, 2H), 2.63 (d, J=15.1 Hz, 1H), 2.38-2.26 (m, 1H), 2.15-1.89 (m, 6H), 1.81-1.69 (m, 2H), 1.65-1.47 (m, 2H).

### Example 22

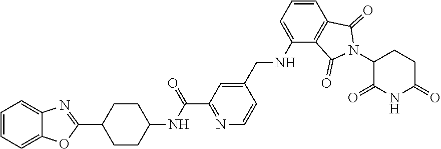 [SMILES: O=C1CCC(N2C(=O)c3cccc(NCc4ccnc(C(=O)NC5CCC(c6nc7ccccc7o6)CC5)c4)c3C2=O)C(=O)N1]

### Preparation Steps

### Step 1

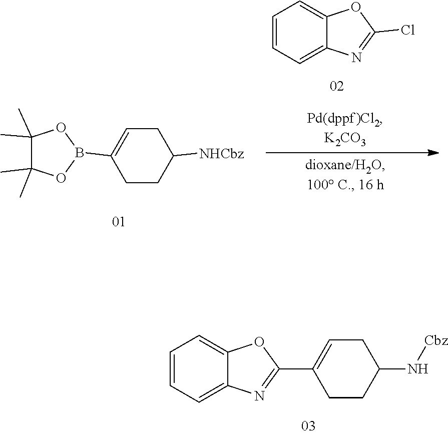 [SMILES: Clc1nc2ccccc2o1 ; CC1(C)OB(C2=CCC(NC(=O)OCc3ccccc3)CC2)OC1(C)C ; O=C(NC1CC=C(c2nc3ccccc3o2)CC1)OCc1ccccc1]

To a 50 mL single-neck flask were added 01 (500 mg, 1.40 mmol), 02 (215 mg, 1.40 mmol), Pd(dppf)Cl₂ (102 mg, 0.14 mmol), K₂CO₃ (580 mg, 4.20 mmol), 1,4-dioxane (8 mL) and water (2 mL), and the mixture was reacted in a nitrogen atmosphere at 100° C. for 16 h, cooled to room temperature, quenched with water (20 mL), and extracted with ethyl acetate (20 mL×3). The organic phase was washed with saturated brine (20 mL×3) and concentrated to dryness by rotary evaporation, and the residue was purified by pre-TLC (petroleum ether:ethyl acetate=5:1) to give the target product as a yellow solid (227 mg, 45.4% yield). LC-MS: 349.1 [M+H]⁺.

### Step 2

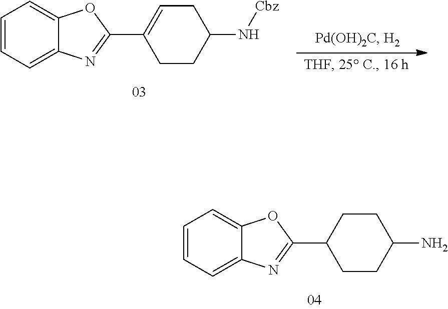 [SMILES: O=C(NC1CC=C(c2nc3ccccc3o2)CC1)OCc1ccccc1 ; NC1CCC(c2nc3ccccc3o2)CC1]

In a 100 mL single-neck round-bottom flask, 03 (227 mg, 0.65 mmol) and palladium hydroxide/carbon (10 mg) were placed in tetrahydrofuran (3 mL). The mixture was reacted in a hydrogen atmosphere at room temperature for 16 h and filtered, and the filtrate was concentrated to dryness by rotary evaporation to give a crude product, which was then purified by pre-TLC (petroleum ether:ethyl acetate=3:1) to give the target product as a white solid (192 mg, 84.6% yield). LC-MS: 217.1 [M+H]⁺.

### Step 3

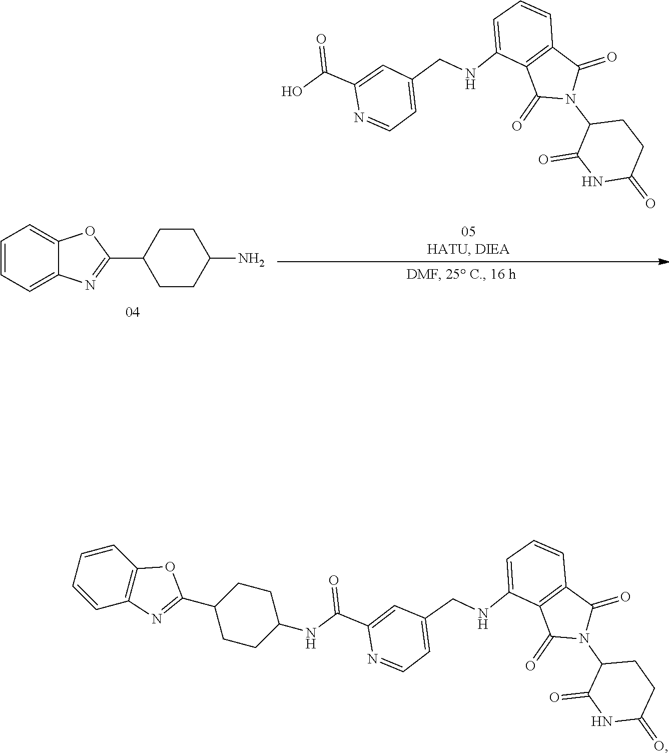 [SMILES: O=C1CCC(N2C(=O)c3cccc(NCc4ccnc(C(=O)O)c4)c3C2=O)C(=O)N1 ; NC1CCC(c2nc3ccccc3o2)CC1 ; O=C1CCC(N2C(=O)c3cccc(NCc4ccnc(C(=O)NC5CCC(c6nc7ccccc7o6)CC5)c4)c3C2=O)C(=O)N1]

In a 100 mL single-neck flask, 04 (90 mg, 0.42 mmol) was dissolved in N,N-dimethylformamide (4 mL), and HATU (236 mg, 0.63 mmol), DIEA (161 mg, 1.26 mmol) and 05 (169 mg, 0.42 mmol) were added. The mixture was stirred at room temperature for 16 h and purified by preparative reversed-phase chromatography to give the target product as a yellow solid (38.81 mg, 43.1% yield). LC-MS: 607.1 [M+H]⁺, 1H NMR (400 MHz, DMSO-d6) δ 11.12 (s, 1H), 8.58 (t, J=6.0 Hz, 2H), 8.03 (d, J=12.8 Hz, 1H), 7.76-7.62 (m, 2H), 7.52 (dt, J=13.5, 5.0 Hz, 3H), 7.35 (dd, J=6.5, 2.9 Hz, 2H), 7.05 (d, J=7.0 Hz, 1H), 6.86 (t, J=8.9 Hz, 1H), 5.10 (dd, J=12.8, 5.3 Hz, 1H), 4.70 (t, J=7.4 Hz, 2H), 3.86 (dd, J=1.5, 7.8 Hz, 1H), 3.02-2.82 (m, 2H), 2.22 (d, J=13.1 Hz, 2H), 2.08 (d, J=4.9 Hz, 1H), 1.95 (d, J=12.0 Hz, 2H), 1.78-1.58 (m, 4H).

### Example 23

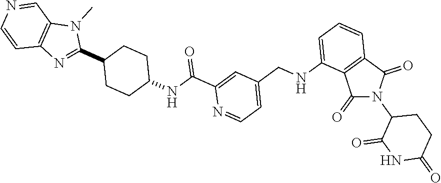

### Preparation Steps

### Step 1

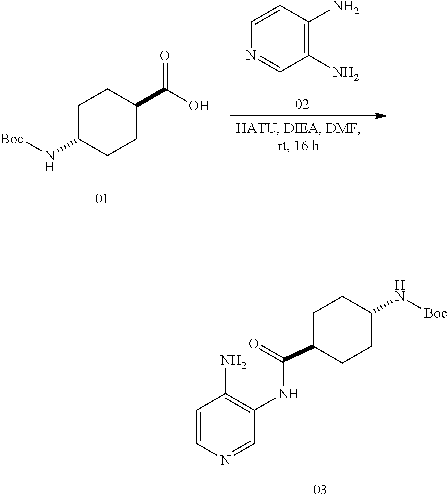 [SMILES: CC(C)(C)OC(=O)N[C@H]1CC[C@H](C(=O)O)CC1 ; Nc1ccncc1N ; CC(C)(C)OC(=O)N[C@H]1CC[C@H](C(=O)Nc2cnccc2N)CC1]

In a 100 mL single-neck flask, 01 (400 mg, 1.64 mmol) was dissolved in N,N-dimethylformamide (5 mL), and 02 (179 mg, 1.64 mmol), HATU (938 g, 2.47 mmol) and N,N-diisopropylethylamine (637 g, 4.93 mmol) were added. The mixture was reacted at room temperature for 16 h, quenched with water (15 mL), and extracted with ethyl acetate (20 mL×3). The organic phase was washed with saturated brine (20 mL×3) and concentrated to dryness by rotary evaporation, and the residue was purified by column chromatography (dichloromethane:methanol=20:1) to give 03 (500 mg, 90.9% yield, brown solid). LC-MS: 335.1 [M+H]⁺.

### Step 2

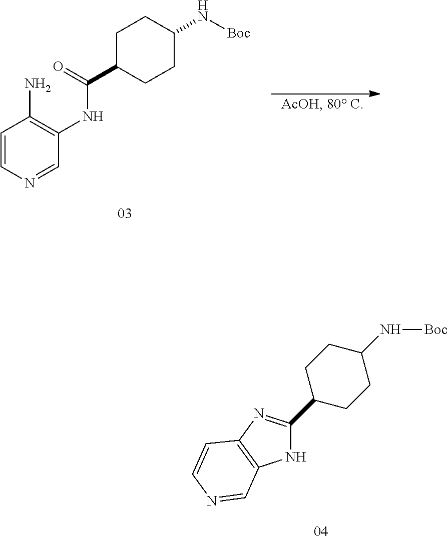

In a 100 mL single-neck flask, 03 (200 mg, 0.598 mmol) was dissolved in acetic acid (2 mL), and the mixture was reacted at 80° C. for 2.5 h, adjusted to pH 8-9 with saturated aqueous sodium bicarbonate solution, and extracted with ethyl acetate (20 mL×3). The organic phase was washed with saturated brine (20 mL×3), dried, and concentrated to give 04 (170 mg, 89.84% yield, yellow solid). LC-MS: 317.4 [M+H]⁺.

### Step 3

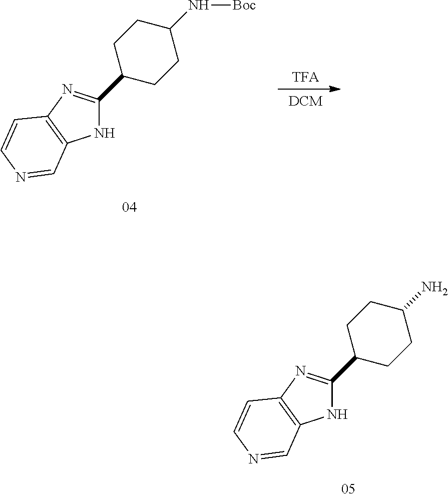

In a 50 mL single-neck flask, 04 (170 mg, 0.537 mmol) was dissolved in dichloromethane (5 mL), and trifluoroacetic acid (1 mL) was added. The mixture was reacted at room temperature for 2 h and concentrated to dryness by rotary evaporation to give crude 05 (130 mg, crude, yellow solid). LC-MS: 217.4 [M+H]⁺.

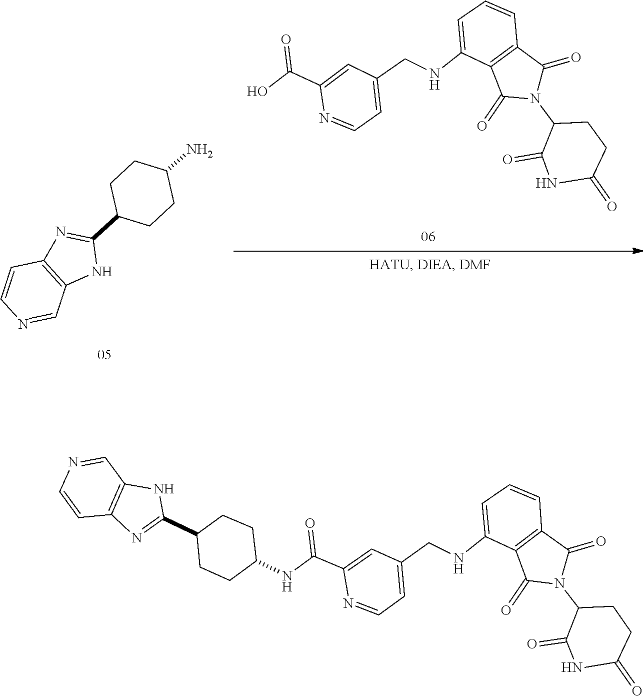 [SMILES: N[C@H]1CC[C@H](c2nc3ccncc3[nH]2)CC1 ; O=C1CCC(N2C(=O)c3cccc(NCc4ccnc(C(=O)O)c4)c3C2=O)C(=O)N1 ; O=C1CCC(N2C(=O)c3cccc(NCc4ccnc(C(=O)N[C@H]5CC[C@H](c6nc7ccncc7[nH]6)CC5)c4)c3C2=O)C(=O)N1]

06 (40 mg, 0.0980 mmol) was dissolved in N,N-dimethylformamide (1 mL) at room temperature, and crude 05 (21 mg, 0.0980 mmol), HATU (56 mg, 0.147 mmol) and N,N-diisopropylethylamine (38 mg, 0.294 mmol) were added successively. The above mixture was stirred at room temperature for 1 h. The reaction mixture was purified by preparative high performance liquid chromatography (formic acid) to give the target product (10.04 mg, 16.9% yield, yellow solid). LC-MS: 607.3 [M+H]⁺.

¹H NMR (400 MHz, DMSO-d₆) δ 12.85 (s, 1H), 11.11 (s, 1H), 8.91 (s, 1H), 8.62-8.52 (m, 2H), 8.30 (s, 1H), 8.04 (s, 1H), 7.64-7.54 (m, 2H), 7.49 (dd, J=16.0, 8.1 Hz, 2H), 7.05 (d, J=7.1 Hz, 1H), 6.87 (d, J=8.5 Hz, 1H), 5.09 (dd, J=12.8, 5.4 Hz, 1H), 4.71 (d, J=6.3 Hz, 2H), 3.94-3.80 (m, 1H), 2.97-2.84 (m, 2H), 2.65-2.53 (m, 2H), 2.18-2.04 (m, 3H), 2.00-1.91 (m, 2H), 1.81-1.56 (m, 4H).

### Example 24

### Synthesis of

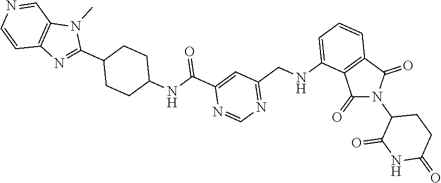 [SMILES: Cn1c(C2CCC(NC(=O)c3cc(CNc4cccc5c4C(=O)N(C4CCC(=O)NC4=O)C5=O)ncn3)CC2)nc2ccncc21]

### Example 25

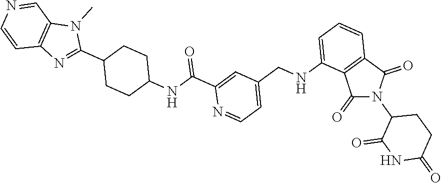 [SMILES: Cn1c(C2CCC(NC(=O)c3cc(CNc4cccc5c4C(=O)N(C4CCC(=O)NC4=O)C5=O)ccn3)CC2)nc2ccncc21]

### Step 1

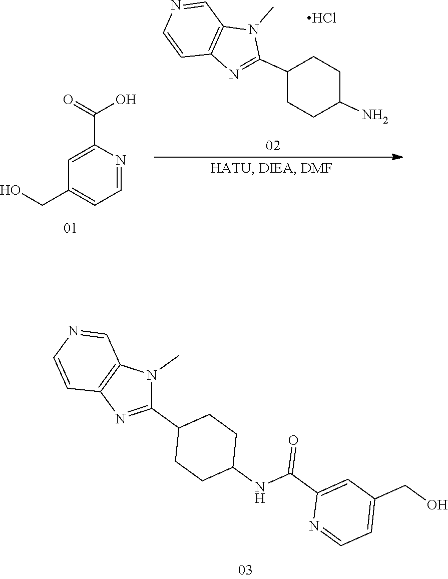 [SMILES: O=C(O)c1cc(CO)ccn1 ; Cn1c(C2CCC(N)CC2)nc2ccncc21 ; Cl ; Cn1c(C2CCC(NC(=O)c3cc(CO)ccn3)CC2)nc2ccncc21]

To a 25 mL single-neck flask were added 01 (40 mg, 0.26 mmol), HATU (200 mg, 0.52 mmol) and DMF (2 mL), followed by 02 (70 mg, 0.26 mmol) and DIEA (102 mg, 0.78 mmol). The mixture was stirred at room temperature for 16 h. After the reaction was completed, the reaction mixture was purified by reversed-phase chromatography (acetonitrile/water=50%) to give the target product as a white solid (180 mg, 70.1% yield). LC-MS: 366.3 [M+H]⁺.

### Step 2

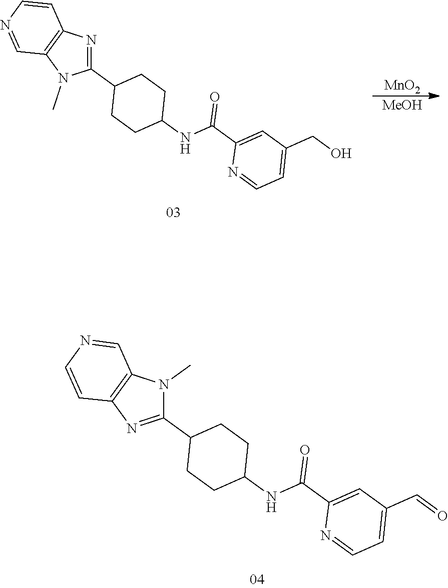 [SMILES: "MeOH" ; Cn1c(C2CCC(NC(=O)c3cc(CO)ccn3)CC2)nc2ccncc21 ; "MnO2" ; Cn1c(C2CCC(NC(=O)c3cc(C=O)ccn3)CC2)nc2ccncc21]

In a 50 mL single-neck flask, 03 (150 mg, 0.4 mmol) was dissolved in methanol (5 mL), and manganese dioxide (715 mg, 8.2 mmol) was added. The mixture was reacted in a nitrogen atmosphere at room temperature for 16 h and filtered, and the filtrate was concentrated to dryness by rotary evaporation to give the target product as a yellow solid (156 mg, 97.9% yield). LC-MS: 364.1 [M+H]⁺.

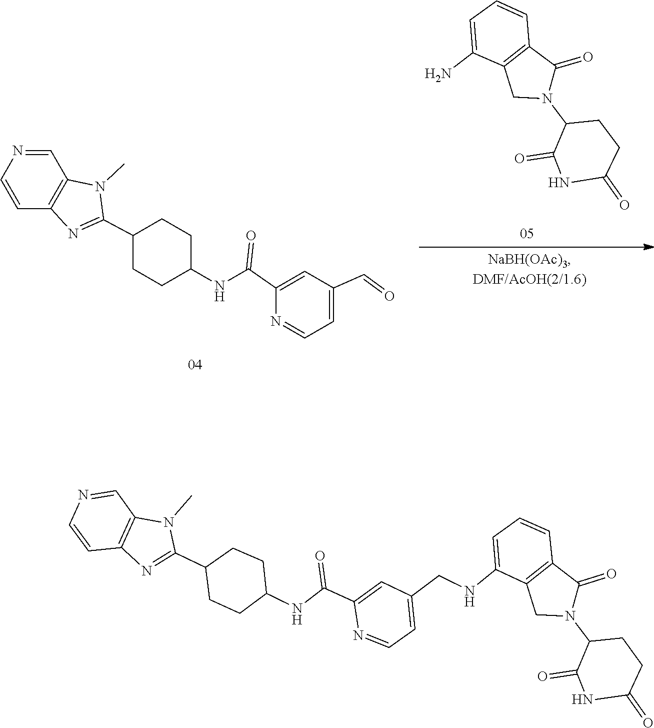 [SMILES: Cn1c(C2CCC(NC(=O)c3cc(C=O)ccn3)CC2)nc2ccncc21 ; Nc1cccc2c1CN(C1CCC(=O)NC1=O)C2=O ; Cn1c(C2CCC(NC(=O)c3cc(CNc4cccc5c4CN(C4CCC(=O)NC4=O)C5=O)ccn3)CC2)nc2ccncc21]

In a 50 mL single-neck flask, 05 (105 mg, 0.4 mmol) was dissolved in N,N-dimethylformamide/acetic acid=(6 mL/4.8 mL), and 04 (147 mg, 0.4 mmol) was added, followed by addition of sodium triacetoxyborohydride (257.5 mg, 1.2 mmol) with cooling in an ice bath. The mixture was stirred at room temperature for 16 h. After being filtered, the reaction mixture was purified by preparative high performance liquid chromatography to give the target product as a yellow solid (12.76 mg, 4.9% yield). LC-MS: 607.1 [M+H]⁺, ¹HNMR (400 MHz, DMSO-d₆) δ 11.02 (s, 1H), 9.32 (s, 1H), 8.59-8.53 (m, 3H), 8.07 (s, 2H), 7.60 (s, 1H), 7.20 (s, 1H), 6.96 (s, 1H), 6.57 (s, 2H), 5.15 (s, 1H), 4.55 (s, 2H), 4.33-4.26 (m, 2H), 4.00 (s, 3H), 2.94 (s, 1H), 2.66 (s, 2H), 2.33 (s, 1H), 2.11-1.90 (m, 6H), 1.81-1.69 (m, 4H).

### Example 26

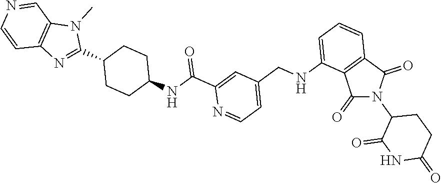 [SMILES: Cn1c2cnccc2nc1[C@H]1CC[C@H](NC(=O)c2cc(CNc3cccc4c3C(=O)N(C3CCC(=O)NC3=O)C4=O)ccn2)CC1]

### Preparation Steps

### Step 1

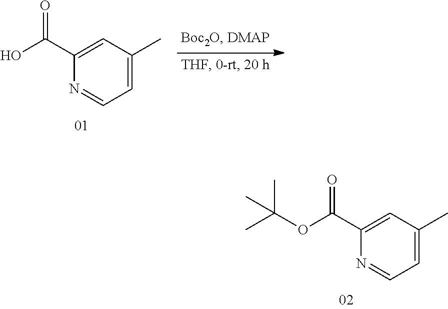

To a 250 mL three-necked flask were added 01 (5 g, 36.46 mmol), THF (50 mL) and DMAP (445 mg, 3.65 mmol), followed by di-tert-butyl dicarbonate (11.9 g, 54.69 mmol) with cooling in an ice bath. The mixture was reacted at room temperature for 12 h, quenched with water (50 mL), and extracted with ethyl acetate (50 mL×2). The organic phase was washed with saturated brine (50 mL×3), dried over anhydrous sodium sulfate, and concentrated to dryness by rotary evaporation, and the residue was purified by column chromatography (ethyl acetate:petroleum ether=1:2) to give a yellow oily liquid (4.37 g, 62.1% yield). LC-MS: 194.1 [M+H]⁺.

### Step 2

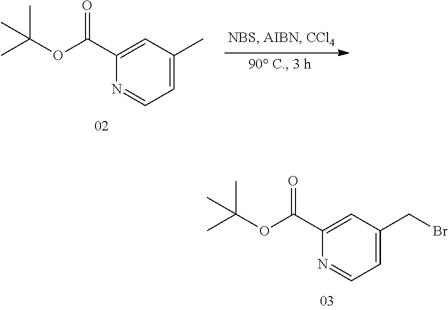

To a 100 mL single-neck flask were added 02 (500 mg, 2.59 mmol), NBS (460 mg, 2.59 mmol), AIBN (85 mg, 0.5174 mmol) and carbon tetrachloride (5 mL), and the mixture was reacted in an argon atmosphere at 90° C. for 3 h, cooled to room temperature, quenched with water (10 mL), and extracted with dichloromethane (10 mL×2). The organic phase was washed with saturated brine (10 mL×3), dried over anhydrous sodium sulfate, and concentrated to dryness by rotary evaporation, and the residue was purified by column-chromatography (ethyl acetate:petroleum ether=1:2) to give a brown oily liquid (386 mg, 54.9% yield). LC-MS: 274.0 [M+H]⁺.

### Step 3

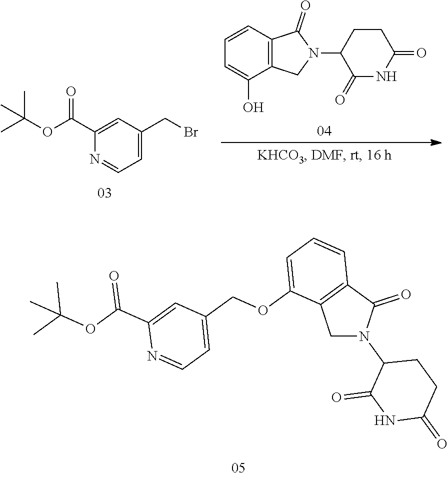 [SMILES: CC(C)(C)OC(=O)c1cc(CBr)ccn1 ; O=C1CCC(N2Cc3c(O)cccc3C2=O)C(=O)N1 ; CC(C)(C)OC(=O)c1cc(COc2cccc3c2CN(C2CCC(=O)NC2=O)C3=O)ccn1]

To a 50 mL single-neck flask were added 03 (104 mg, 0.384 mmol), 04 (100 mg, 0.384 mmol), potassium bicarbonate (115 mg, 1.152 mmol) and DMF (5 mL), and the mixture was reacted at 30° C. for 12 h, quenched with water (10 mL), and extracted with ethyl acetate (10 mL×2). The organic phase was washed with saturated brine (10 mL×3), dried over anhydrous sodium sulfate, and concentrated to dryness by rotary evaporation, and the residue was purified by column chromatography (methanol:dichloromethane=1:10) to give a brown solid (69 mg, 39.8% yield). LC-MS: 452.4 [M+H]⁺.

### Step 4

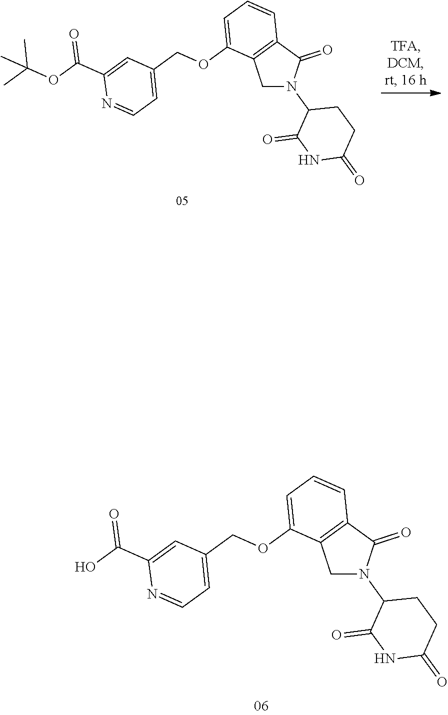 [SMILES: CC(C)(C)OC(=O)c1cc(COc2cccc3c2CN(C2CCC(=O)NC2=O)C3=O)ccn1 ; O=C1CCC(N2Cc3c(OCc4ccnc(C(=O)O)c4)cccc3C2=O)C(=O)N1]

To a 50 mL single-neck flask were added 05 (30 mg, 0.066 mmol), TFA (2 mL) and DCM (2 mL), and the mixture was reacted at room temperature for 2 h and concentrated to dryness by rotary evaporation to give a brown oily liquid (69 mg, crude). LC-MS: 396.3 [M+H]⁺.

### Step 5

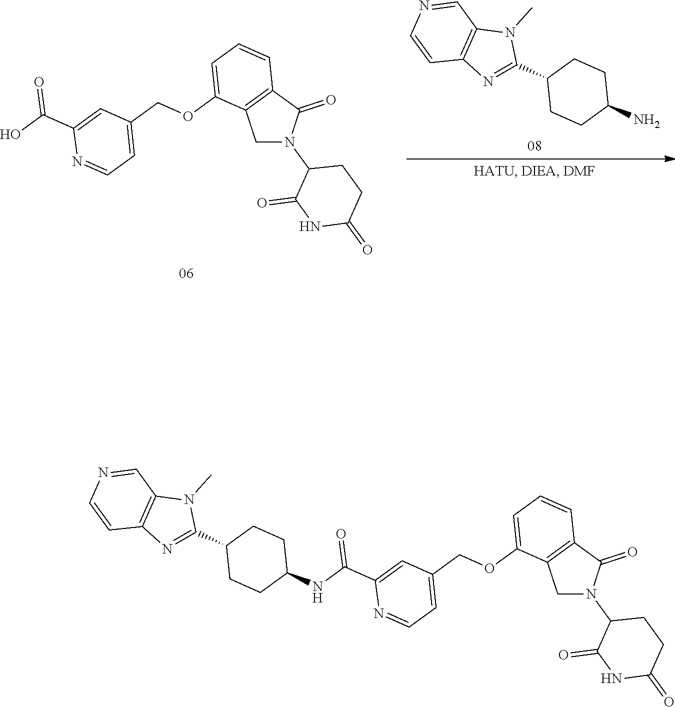 [SMILES: O=C1CCC(N2Cc3c(OCc4ccnc(C(=O)O)c4)cccc3C2=O)C(=O)N1 ; Cn1c2cnccc2nc1[C@H]1CC[C@H](N)CC1 ; Cn1c2cnccc2nc1[C@H]1CC[C@H](NC(=O)c2cc(COc3cccc4c3CN(C3CCC(=O)NC3=O)C4=O)ccn2)CC1]

To a 50 mL single-neck flask were added 06 (30 mg, 0.066 mmol), 08(15 mg, 0.0683 mmol), HATU (43.34 mg, 0.114 mmol), DIEA (29.46 mg, 0.228 mmol) and DMF (2 mL), and the reaction mixture was reacted at room temperature for 12 h and purified by preparative high performance liquid chromatography to give a white solid (1.37 mg, 3% yield). LC-MS: 608.3 [M+H]⁺. 1H NMR (400 MHz, DMSO-d6) δ 11.00 (s, 1H), 9.05 (s, 0.62H), 8.68-8.64 (m, 2H), 8.37 (s, 0.51H), 8.14 (s, 1H), 7.72-7.71 (m, 2H), 7.50 (t, J=4.9 Hz, 1H), 7.37 (d, J=3.8 Hz, 1H), 7.29 (d, J=3.8 Hz, 1H), 7.20-7.07 (m, 1H), 5.46 (s, 2H), 5.14 (dd, J=12.9, 5.3 Hz, 1H), 4.51 (d, J=17.6 Hz, 1H), 4.35 (d, J=17.0 Hz, 1H), 3.94 (s, 4H), 3.07-2.90 (m, 2H), 2.08-1.99 (m, 4H), 1.81-1.67 (m, 4H).

### Example 27

### Synthesis of

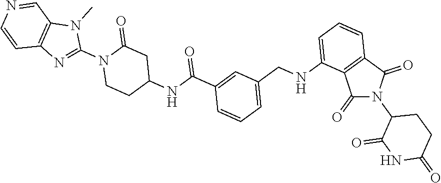 [SMILES: Cn1c(N2CCC(NC(=O)c3cccc(CNc4cccc5c4C(=O)N(C4CCC(=O)NC4=O)C5=O)c3)CC2=O)nc2ccncc21]

### Example 28

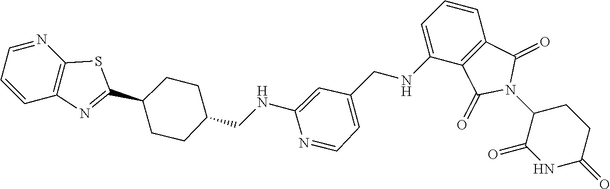 [SMILES: O=C1CCC(N2C(=O)c3cccc(NCc4ccnc(NC[C@H]5CC[C@H](c6nc7cccnc7s6)CC5)c4)c3C2=O)C(=O)N1]

### Preparation Steps

### Step 1

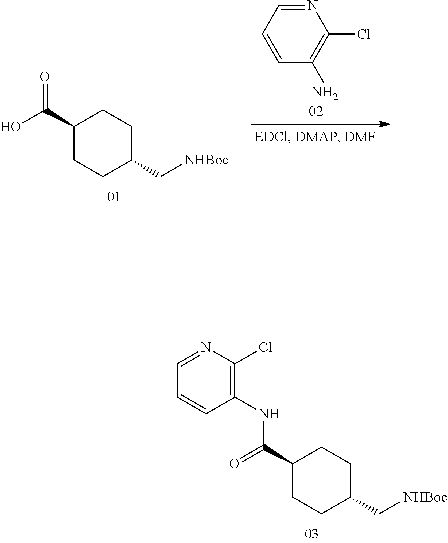 [SMILES: CC(C)(C)OC(=O)NC[C@H]1CC[C@H](C(=O)O)CC1 ; Nc1cccnc1Cl ; CC(C)(C)OC(=O)NC[C@H]1CC[C@H](C(=O)Nc2cccnc2Cl)CC1]

01 (3 g, 11.67 mmol), 02 (2.24 g, 17.51 mmol), 1-ethyl-3(3-dimethylpropylamine)carbodiimide (2.89 g, 15.18 mmol) and 4-dimethylaminopyridine (1.85 g, 15.18 mmol) were dissolved in N,N-dimethylformamide (15 mL) at room temperature. The reaction mixture was stirred at room temperature for 2 h. Water (20 mL) was added, and a solid precipitated. The solid was collected by filtration and slurried with anhydrous ether to give the target product as a white solid (1.3 g, 30.4% yield). LC-MS: 312.1 [M+H−56]⁺.

### Step 2

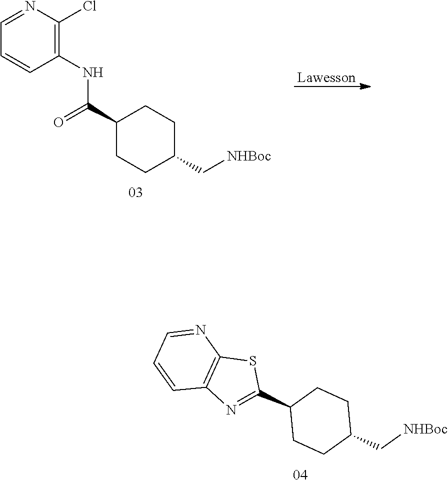 [SMILES: CC(C)(C)OC(=O)NC[C@H]1CC[C@H](C(=O)Nc2cccnc2Cl)CC1 ; "Lawesson" ; CC(C)(C)OC(=O)NC[C@H]1CC[C@H](c2nc3cccnc3s2)CC1]

03 (1.3 g, 3.54 mmol) was dissolved in tetrahydrofuran (10 mL) at room temperature, and Lawesson's reagent (2.15 g, 5.31 mmol) was added. The reaction mixture was stirred in an argon atmosphere at 60° C. for 16 h. Water (50 mL) was added, followed by extraction with ethyl acetate (50 mL×3). The organic phase was washed with saturated brine (50 mL), dried over anhydrous sodium sulfate, and filtered, and the filtrate was concentrated to dryness by rotary evaporation. The residue was purified through a silica gel column (petroleum ether:ethyl acetate=100:0 to 70:30) to give the target product as a white solid (850 mg, 69.2% yield). LC-MS: 348.1 [M+H]⁺.

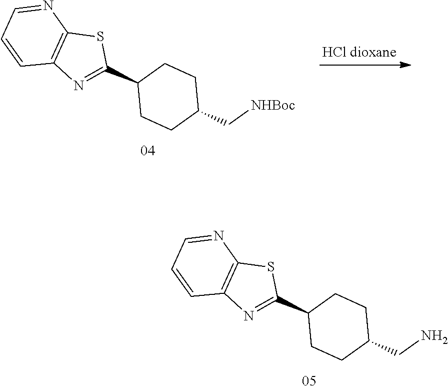 [SMILES: CC(C)(C)OC(=O)NC[C@H]1CC[C@H](c2nc3cccnc3s2)CC1 ; NC[C@H]1CC[C@H](c2nc3cccnc3s2)CC1]

04 (400 mg, 1.15 mmol) was dissolved in dichloromethane (4 mL) at room temperature, followed by addition of hydrochloric acid in 1,4-dioxane (4 mL). The reaction mixture was stirred at room temperature for 2 h and concentrated to dryness by rotary evaporation to give the target product as a white solid (325 mg, 99.5% yield). LC-MS: 248.1 [M+H]⁺.

### Step 4

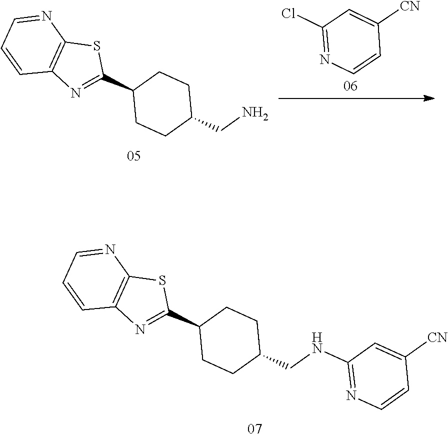 [SMILES: NC[C@H]1CC[C@H](c2nc3cccnc3s2)CC1 ; N#Cc1ccnc(Cl)c1 ; N#Cc1ccnc(NC[C@H]2CC[C@H](c3nc4cccnc4s3)CC2)c1]

05 (250 mg, 1.01 mmol), 06 (140 mg, 1.01 mmol), 4,5-bis(diphenylphosphino)-9,9-dimethylxanthene (76 mg, 0.13 mmol), bis(dibenzylideneacetone)palladium (29 mg, 0.05 mmol) and cesium carbonate (657 mg, 2.02 mmol) were dissolved in anhydrous 1,4-dioxane (5 mL) at room temperature. The reaction mixture was stirred in an argon atmosphere at 80° C. for 3 h and concentrated to dryness by rotary evaporation, and the residue was passed through a reversed-phase column (C18, water:acetonitrile=100:0 to 40:60, 0.1% formic acid) to separate the target product as a white solid (50 mg, 14.2% yield). LC-MS: 350.1 [M+H]⁺.

### Step 5

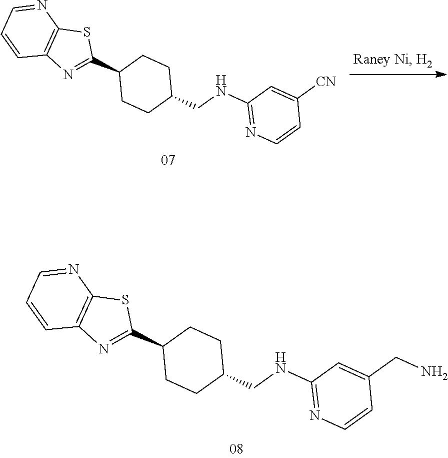 [SMILES: N#Cc1ccnc(NC[C@H]2CC[C@H](c3nc4cccnc4s3)CC2)c1 ; NCc1ccnc(NC[C@H]2CC[C@H](c3nc4cccnc4s3)CC2)c1]

07 (50 mg, 0.14 mmol) was dissolved in tetrahydrofuran (2 mL) at room temperature, and Raney nickel (20 mg) and one drop of ammonia solution were added at room temperature. The mixture was purged twice with hydrogen, stirred in a hydrogen atmosphere at room temperature for 6 h, and filtered, and the filtrate was concentrated to dryness by rotary evaporation to give the target product as a colorless sticky liquid (50 mg, 99.1% yield). LC-MS: 354.1 [M+H]⁺.

### Step 6

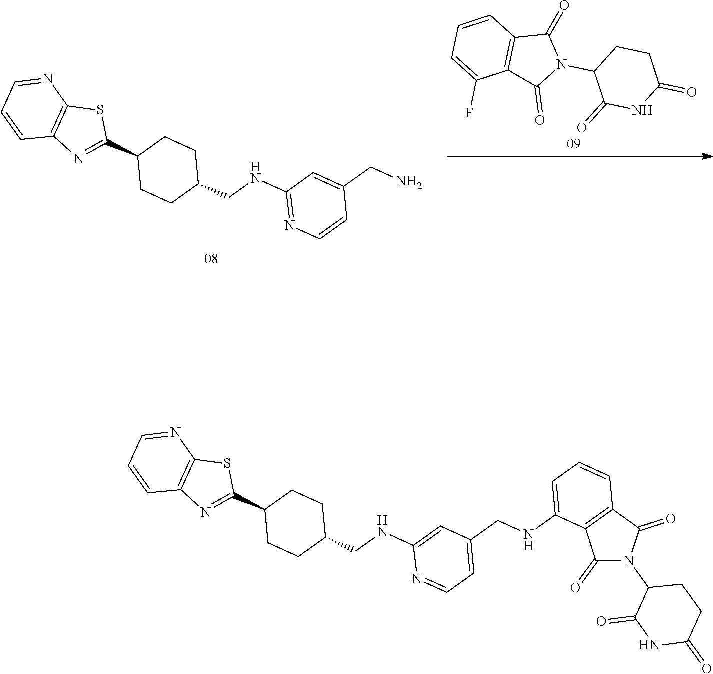 [SMILES: NCc1ccnc(NC[C@H]2CC[C@H](c3nc4cccnc4s3)CC2)c1 ; O=C1CCC(N2C(=O)c3cccc(F)c3C2=O)C(=O)N1 ; O=C1CCC(N2C(=O)c3cccc(NCc4ccnc(NC[C@H]5CC[C@H](c6nc7cccnc7s6)CC5)c4)c3C2=O)C(=O)N1]

08 (50 mg, 0.14 mmol), 09 (39 mg, 0.14 mmol) and N,N-diisopropylethylamine (54 mg, 0.42 mmol) were dissolved in dimethyl sulfoxide (1 mL) at room temperature. The reaction mixture was stirred at 110° C. for 16 h. The mixture was cooled to 20° C. and then purified by preparative chromatography (C18, 5-100% acetonitrile/water as mobile phase, 0.1% formic acid) to separate the target product as a yellow solid (2.89 mg, 3.39% yield). LC-MS: [M+H]+ 610.4.

¹H NMR (400 MHz, DMSO-d₆) δ 11.10 (s, 1H), 8.56 (s, 1H), 8.31 (d, J=7.7 Hz, 1H), 7.88 (d, J=4.9 Hz, 1H), 7.59-7.48 (m, 2H), 7.27-7.17 (m, 1H), 7.04 (d, J=7.1 Hz, 1H), 6.85 (d, J=8.4 Hz, 1H), 6.63-6.53 (m, 1H), 6.45-6.36 (m, 2H), 5.08 (dd, J=12.7, 5.1 Hz, 1H), 4.43 (d, J=5.8 Hz, 2H), 3.13-3.08 (m, 3H), 2.94-2.84 (m, 1H), 2.59 (d, J=20.9 Hz, 2H), 2.17 (d, J=11.0 Hz, 2H), 2.09-2.00 (m, 1H), 1.89 (d, J=11.1 Hz, 2H), 1.60-1.48 (m, 3H), 1.12 (dd, J=24.4, 12.0 Hz, 2H).

### Example 29

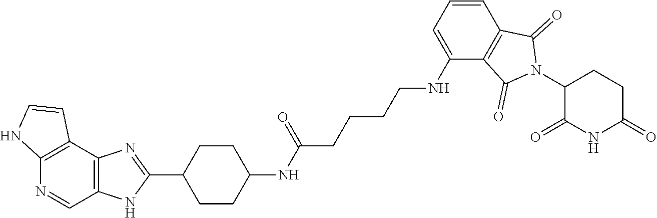 [SMILES: O=C1CCC(N2C(=O)c3cccc(NCCCCC(=O)NC4CCC(c5nc6c(cnc7[nH]ccc76)[nH]5)CC4)c3C2=O)C(=O)N1]

### Preparation Steps

### Step 1

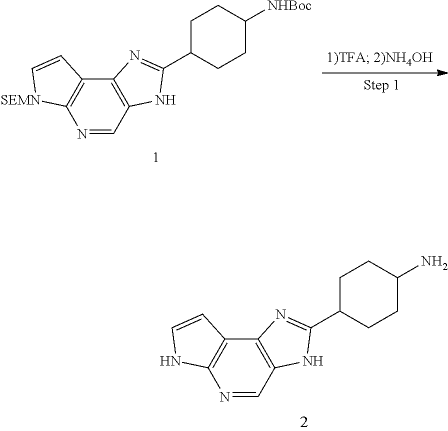 [SMILES: CC(C)(C)OC(=O)NC1CCC(c2nc3c(cnc4c3ccn4COCC[Si](C)(C)C)[nH]2)CC1 ; NC1CCC(c2nc3c(cnc4[nH]ccc43)[nH]2)CC1]

To a 100 mL single-neck flask were added 10 (200 mg, 0.41 mmol), trifluoroacetic acid (3 mL) and dichloromethane (10 mL), and the mixture was reacted at room temperature for 16 h and concentrated to give a crude product as a yellow oil (150 mg), followed by addition of ammonia (2 mL) and dioxane (10 mL). The mixture was reacted at 70° C. for 16 h and concentrated to dryness by rotary evaporation, and the residue was purified by Prep-HPLC (ACN-H₂O, TFA) to give the target product as a yellow oil (53 mg, 51.21%). LC-MS: 256 [M+H]⁺.

### Step 2

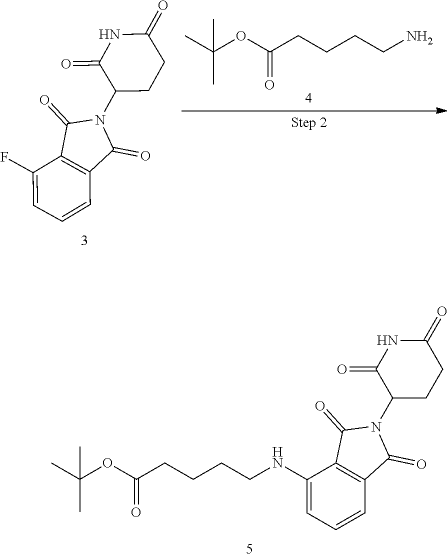

3 (500 mg, 1.81 mmol), 4 (376 mg, 2.17 mmol) and N-methylpyrrolidone (5 mL) were added to a 100 mL single-neck flask and mixed well by stirring, and DIEA (468 mg, 3.62 mmol) was added. The mixture was reacted at 110° C. for 18 h and concentrated to dryness by rotary evaporation, and the residue was purified by column chromatography to give the target product as a yellowish-green solid (341 mg, 43.91%). LC-MS: 430 [M+H]⁺.

### Step 3

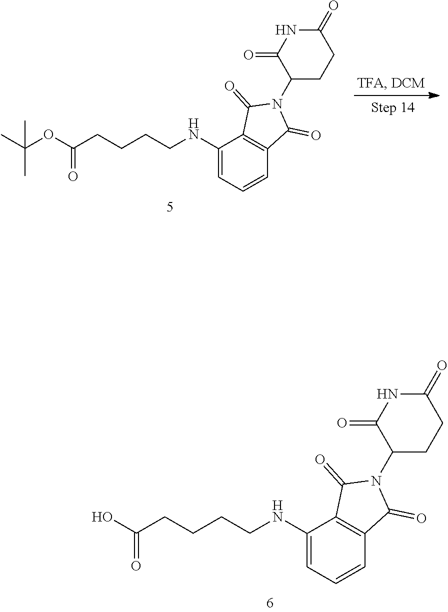 [SMILES: CC(C)(C)OC(=O)CCCCNc1cccc2c1C(=O)N(C1CCC(=O)NC1=O)C2=O ; O=C(O)CCCCNc1cccc2c1C(=O)N(C1CCC(=O)NC1=O)C2=O]

In a 100 mL single-neck flask, 5 (341 mg, 0.79 mmol) was dissolved in DCM (10 mL), and TFA (1 mL) was added. The mixture was reacted at room temperature for 16 h and concentrated to dryness by rotary evaporation, and the residue was purified by column chromatography to give the target product as a yellowish-green solid (210 mg, 71.07%). LC-MS: 374 [M+H]⁺.

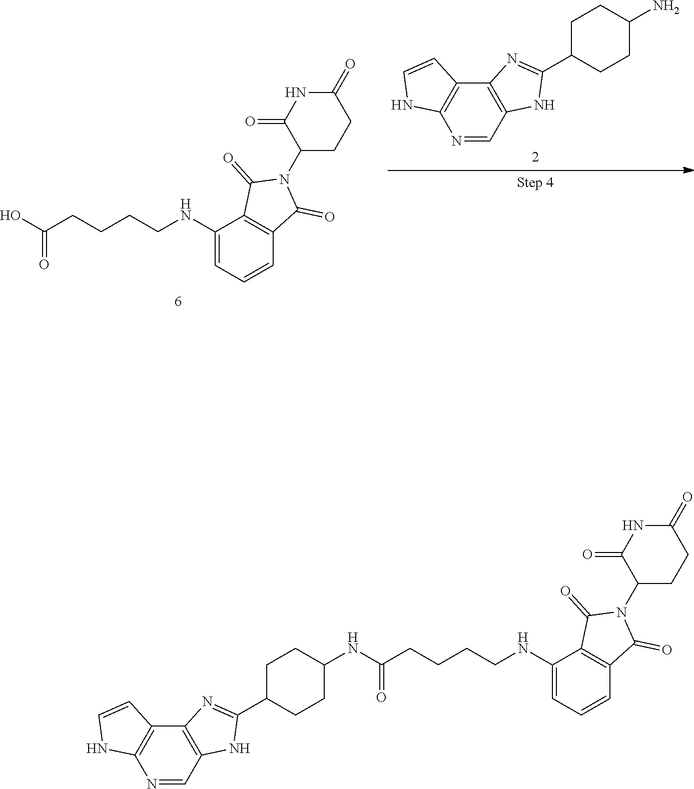 [SMILES: O=C(O)CCCCNc1cccc2c1C(=O)N(C1CCC(=O)NC1=O)C2=O ; NC1CCC(c2nc3c(cnc4[nH]ccc43)[nH]2)CC1 ; O=C1CCC(N2C(=O)c3cccc(NCCCCC(=O)NC4CCC(c5nc6c(cnc7[nH]ccc76)[nH]5)CC4)c3C2=O)C(=O)N1]

Compound 6 (75 mg, 0.2 mmol), compound 2 (53 mg, 0.20 mmol), HATU (114 mg, 0.3 mmol) and DIEA (77.4 mg, 0.6 mmol) were added to a flask containing dichloromethane, and the mixture was stirred at room temperature overnight. After the solvent was removed by rotary evaporation, the crude product was purified by Prep-HPLC to give the product as a yellow solid (15 mg, 12.29%). LC-MS: 611 [M+H]⁺, 1NMR (400 MHz, DMSO) δ 11.61 (s, 1H), 11.08 (s, 1H), 8.42 (s, 1H), 7.75 (d, J=7.7 Hz, 1H), 7.60-7.52 (m, 1H), 7.34 (s, 1H), 7.07 (d, J=8.6 Hz, 1H), 6.99 (d, J=7.0 Hz, 1H), 6.58-6.50 (m, 2H), 5.02 (dd, J=12.9, 5.3 Hz, 1H), 3.65-3.53 (m, 1H), 2.90-2.79 (m, 2H), 2.55 (d, J=16.8 Hz, 2H), 2.08 (s, 4H), 2.02-1.97 (m, 1H), 1.89 (d, J=10.4 Hz, 2H), 1.74-1.62 (m, 2H), 1.58 (d, J=28.0 Hz, 5H), 1.38-1.19 (m, 3H).

### Example 30

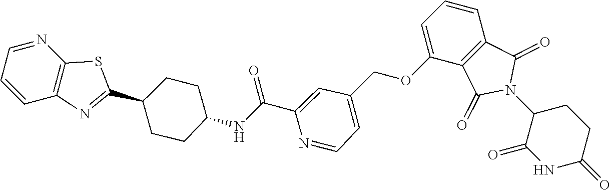 [SMILES: O=C1CCC(N2C(=O)c3cccc(OCc4ccnc(C(=O)N[C@H]5CC[C@H](c6nc7cccnc7s6)CC5)c4)c3C2=O)C(=O)N1]

### Preparation Steps

### Step 1

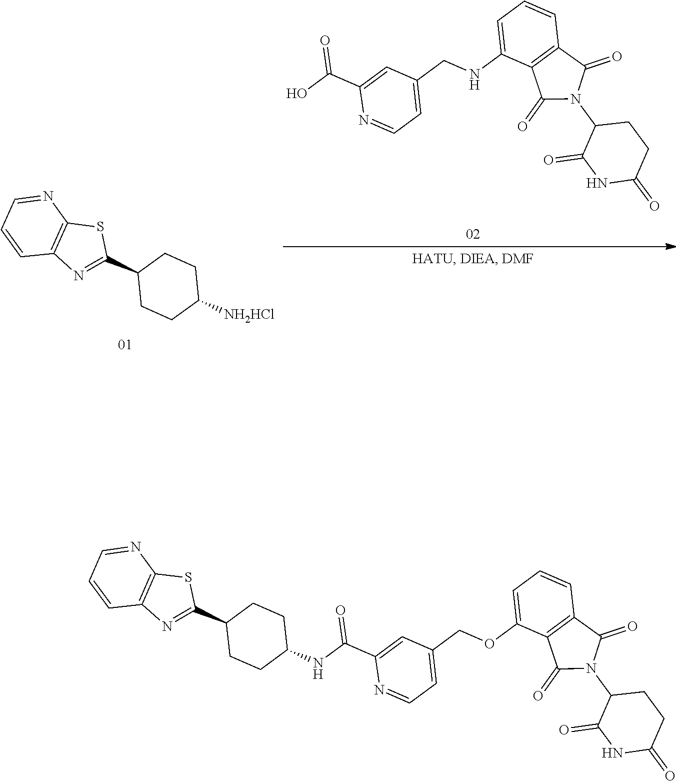

01 (20.0 mg, 0.074 mmol), 02 (37.7 mg, 0.074 mmol) and N,N-diisopropylethylamine (38.3 mg, 0.296 mmol) were mixed in anhydrous N,N-dimethylformamide (1 mL) at room temperature, and (7-azabenzotriazole)-tetramethyluronium hexafluorophosphate (42.2 mg, 0.111 mmol) was added in batches with stirring at room temperature. The reaction mixture was stirred at room temperature for 1 h. The mixture was purified by preparative chromatography (C18, 5-100% acetonitrile/water as mobile phase, 0.1% formic acid) to separate the target product as a white solid (10.4 mg, 22.9% yield). LC-MS: 611.4[M+H]⁺, ¹H NMR (400 MHz, DMSO-d₆) δ 10.99 (s, 1H), 8.67 (d, J=5.0 Hz, 1H), 8.62 (d, J=8.5 Hz, 1H), 8.58 (dd, J=4.6, 1.4 Hz, 1H), 8.35 (dd, J=8.2, 1.5 Hz, 1H), 8.13 (s, 1H), 7.71 (d, J=5.0 Hz, 1H), 7.56 (dd, J=8.2, 4.7 Hz, 1H), 7.50 (t, J=7.8 Hz, 1H), 7.36 (d, J=7.4 Hz, 1H), 7.28 (d, J=8.1 Hz, 1H), 5.46 (s, 2H), 5.14 (dd, J=13.3, 5.1 Hz, 1H), 4.51 (d, J=17.5 Hz, 1H), 4.35 (d, J=17.4 Hz, 1H), 3.97-3.87 (m, 1H), 3.18-3.09 (m, 1H), 2.98-2.87 (m, 1H), 2.64-2.56 (m, 1H), 2.46-2.41 (m, 1H), 2.28-2.20 (m, 2H), 2.05-1.96 (m, 3H), 1.81-1.63 (m, 4H).

### Example 31

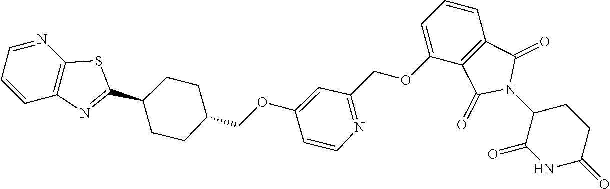 [SMILES: O=C1CCC(N2C(=O)c3cccc(OCc4cc(OC[C@H]5CC[C@H](c6nc7cccnc7s6)CC5)ccn4)c3C2=O)C(=O)N1]

### Preparation Steps

### Step 1

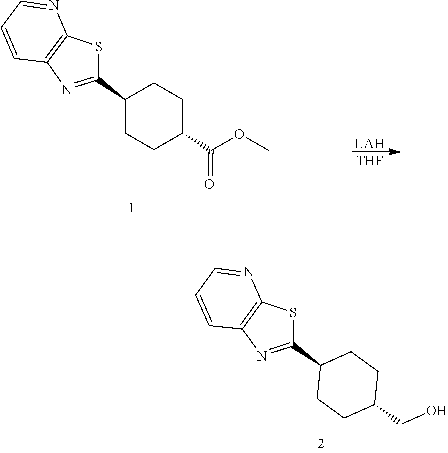 [SMILES: COC(=O)[C@H]1CC[C@H](c2nc3cccnc3s2)CC1 ; OC[C@H]1CC[C@H](c2nc3cccnc3s2)CC1]

1 (2.00 g, 7.24 mmol) was dissolved in anhydrous tetrahydrofuran (40 mL), and lithium aluminum hydride (1.37 g, 36.2 mmol) was added in batches with stirring at room temperature. The reaction mixture was stirred at 60° C. for 16 h. Water (1.5 mL), 10% aqueous sodium hydroxide solution (1.5 mL) and water (5 mL) were added slowly successively at 0° C. The mixture was filtered, and the filter cake was washed three times with tetrahydrofuran (20 mL). The filtrate was concentrated to dryness by rotary evaporation. The residue was purified through a flash silica gel column (petroleum ether:ethyl acetate=0 to 50%) to give the target product as a pale yellow solid (1.20 g, 66.8% yield). LC-MS: 249.4 [M+H]⁺.

### Step 2

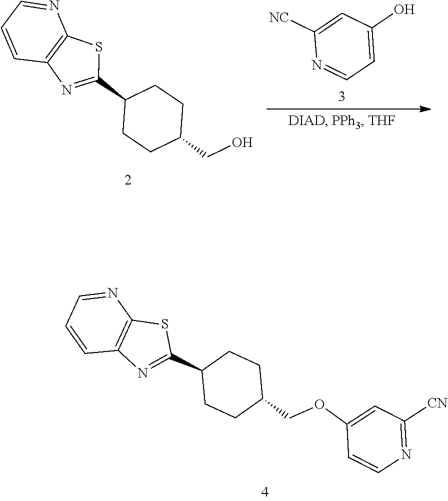 [SMILES: OC[C@H]1CC[C@H](c2nc3cccnc3s2)CC1 ; N#Cc1cc(O)ccn1 ; N#Cc1cc(OC[C@H]2CC[C@H](c3nc4cccnc4s3)CC2)ccn1]

2 (150 mg, 0.60 mmol), 3 (86 mg, 0.72 mmol) and triphenylphosphine (314 mg, 1.20 mmol) were dissolved in anhydrous tetrahydrofuran (15 mL), and diisopropyl azodicarboxylate (242 mg, 1.20 mmol) was added with stirring at 0° C. The reaction mixture was stirred at 0° C. for 16 h and concentrated to dryness by rotary evaporation, and the residue was purified through a reversed-phase column (C18, 10-100% acetonitrile/water as mobile phase, 0.1% formic acid) to give the target product as a white solid (130 mg, 61.9% yield). LC-MS: 351.2 [M+H]⁺.

### Step 3

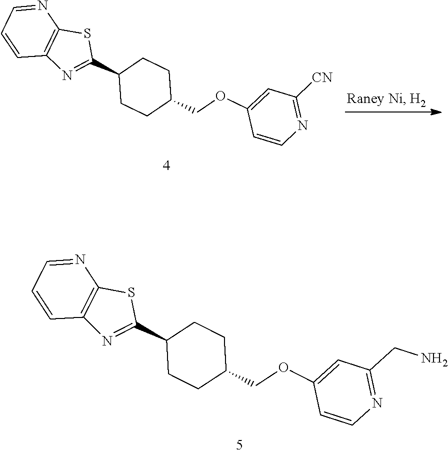 [SMILES: N#Cc1cc(OC[C@H]2CC[C@H](c3nc4cccnc4s3)CC2)ccn1 ; NCc1cc(OC[C@H]2CC[C@H](c3nc4cccnc4s3)CC2)ccn1]

4 (130 mg, 0.369 mmol) was dissolved in tetrahydrofuran (10 mL) at room temperature, and Raney nickel (100 mg) was added at room temperature. The reaction mixture was stirred in a hydrogen atmosphere at room temperature for 6 h and filtered, and the filtrate was concentrated to dryness by rotary evaporation to the target product as a colorless sticky liquid (80 mg, crude). LC-MS: 355.3 [M+H]⁺.

### Step 4

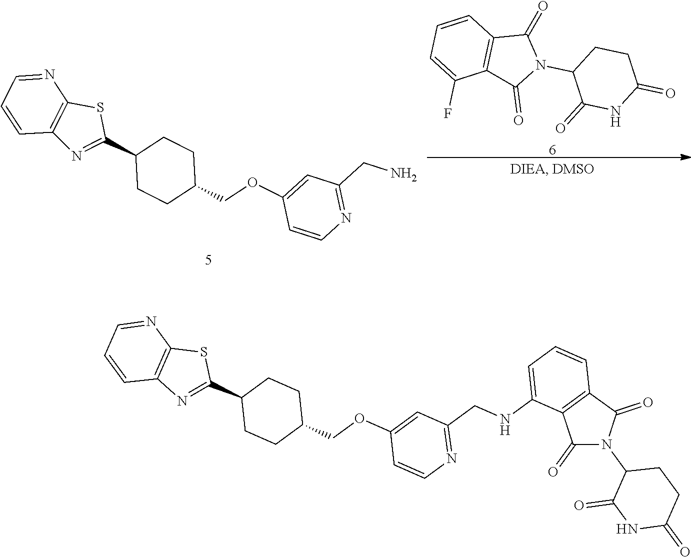 [SMILES: NCc1cc(OC[C@H]2CC[C@H](c3nc4cccnc4s3)CC2)ccn1 ; O=C1CCC(N2C(=O)c3cccc(F)c3C2=O)C(=O)N1 ; O=C1CCC(N2C(=O)c3cccc(NCc4cc(OC[C@H]5CC[C@H](c6nc7cccnc7s6)CC5)ccn4)c3C2=O)C(=O)N1]

5 (80 mg, 0.22 mmol), 6 (63 mg, 0.22 mmol) and N,N-diisopropylethylamine (38 mg, 0.44 mmol) were dissolved in dimethyl sulfoxide (2 mL) at room temperature. The reaction mixture was stirred at 120° C. for 2 h. The mixture was cooled to room temperature and then purified by preparative chromatography (C18, 5-100% acetonitrile/water as mobile phase, 0.1% formic acid) to separate the target product as a yellow solid (10.08 mg, 7.2% yield). LC-MS: 611.4 [M+H]⁺,

¹H NMR (400 MHz, DMSO) δ 11.10 (s, 1H), 8.57 (dd, J=4.6, 1.4 Hz, 1H), 8.37 (d, J=5.7 Hz, 1H), 8.33 (dd, J=8.2, 1.4 Hz, 1H), 7.60-7.53 (m, 2H), 7.45 (t, J=5.7 Hz, 1H), 7.06 (d, J=7.0 Hz, 1H), 7.02 (d, J=8.6 Hz, 1H), 6.98 (d, J=2.1 Hz, 1H), 6.92 (dd, J=5.8, 2.4 Hz, 1H), 5.08 (dd, J=12.8, 5.3 Hz, 1H), 4.57 (d, J=5.7 Hz, 2H), 3.93 (d, J=6.3 Hz, 2H), 3.13 (tt, J=12.0, 3.3 Hz, 1H), 2.94-2.84 (m, 1H), 2.62-2.53 (m, 2H), 2.25-2.17 (m, 2H), 2.08-2.00 (m, 1H), 2.00-1.92 (m, 2H), 1.90-1.81 (m, 1H), 1.69-1.58 (m, 2H), 1.33-1.22 (m, 2H).

### Example 32

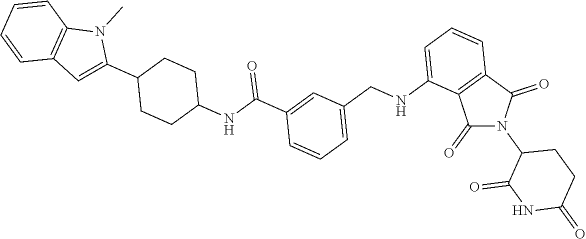 [SMILES: Cn1c(C2CCC(NC(=O)c3cccc(CNc4cccc5c4C(=O)N(C4CCC(=O)NC4=O)C5=O)c3)CC2)cc2ccccc21]

### Preparation Steps

### Step 1

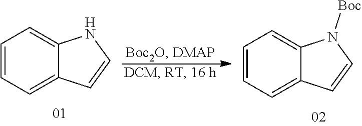 [SMILES: c1ccc2[nH]ccc2c1 ; "Boc2O, DMAPDCM, RT, 16 h" ; CC(C)(C)OC(=O)n1ccc2ccccc21]

In a 250 mL single-neck flask, 01 (3 g, 25.64 mmol) was dissolved in DCM (30 mL), and di-tert-butyl dicarbonate (6 g, 25.64 mmol) and DMAP (600 mg, 4.92 mmol) were added. The mixture was reacted in a nitrogen atmosphere at room temperature for 16 h, quenched with water (20 mL), and extracted with DCM (50 mL×3). The organic phase was washed with saturated brine (20 mL×3) and concentrated to dryness by rotary evaporation, and the residue was purified by column chromatography (ethyl acetate/petroleum ether=1:10) to give the target product as a yellow solid (4.5 g, 80.9% yield). LC-MS: 218.1 [M+H]⁺.

### Step 2

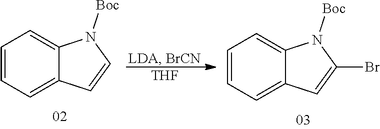 [SMILES: CC(C)(C)OC(=O)n1ccc2ccccc21 ; "LDA, BrCNTHF" ; CC(C)(C)OC(=O)n1c(Br)cc2ccccc21]

In a 500 mL three-necked round-bottom flask, 02 (4.5 g, 20.74 mmol) was dissolved in tetrahydrofuran (45 mL), and LDA (2 mol/L, 11.7 mL) was slowly added dropwise at −75° C. After the mixture was reacted at −75° C. for 30 min, cyanogen bromide (8.1 g, 78.59 mmol) was added. The mixture was slowly warmed to room temperature, reacted for 16 h, quenched with water (20 mL), and extracted with ethyl acetate (40 mL). The organic phase was washed with saturated brine (20 mL×3) and concentrated to dryness by rotary evaporation, and the residue was purified by column chromatography (ethyl acetate/petroleum ether=1:10) to give the product as a yellow solid (2.6 g, 58% yield). LC-MS: 296.0/298.0 [M+H]⁺.

### Step 3

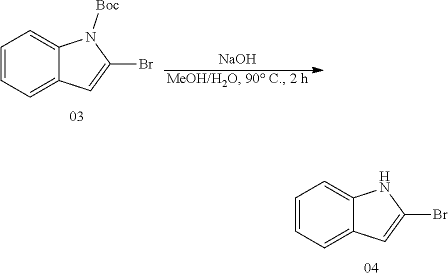 [SMILES: CC(C)(C)OC(=O)n1c(Br)cc2ccccc21 ; "NaOHMeOH/H2O, 90° C., 2 h" ; Brc1cc2ccccc2[nH]1]

In a 100 mL single-neck flask, 03 (2.6 g, 8.81 mmol) was dissolved in MeOH (13 mL) and H₂O (13 mL), and NaOH (1.67 g, 17.62 mmol) was added. The mixture was stirred at 90° C. for 2 h, cooled to room temperature, quenched with water (20 mL), and extracted with ethyl acetate (40 mL). The organic phase was washed with saturated brine (20 mL×3) and concentrated to dryness by rotary evaporation, and the residue was purified by column chromatography (ethyl acetate/petroleum ether=1:5) to give the product as a yellow solid (1.1 g, 42.3% yield). LC-MS: 196.0/198.0 [M+H]⁺.

### Step 4

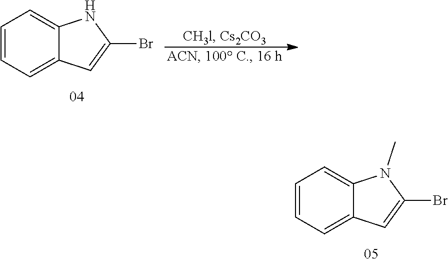

In a 250 mL single-neck flask, 04 (1.1 g, 5.67 mmol) was dissolved in acetonitrile (11 mL), and CH₃I (8.8 mL) and Cs₂CO₃ (5.5 g, 16.88 mmol) were added. The mixture was stirred at 100° C. for 16 h, cooled to room temperature, quenched with water (20 mL), and extracted with ethyl acetate (40 mL). The organic phase was washed with saturated brine (20 mL×3) and concentrated to dryness by rotary evaporation, and the residue was purified by column chromatography (ethyl acetate/petroleum ether=1:6) to give the product as a yellow solid (301 mg, 27.3% yield). LC-MS: 210.0/212.0 [M+H]⁺.

### Step 5

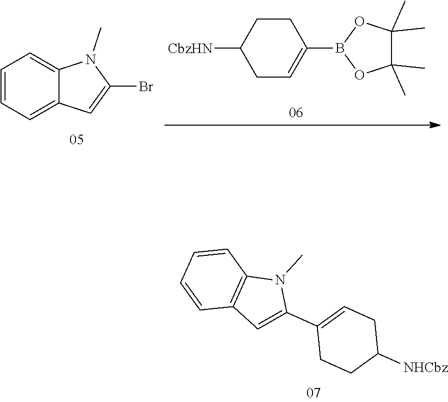 [SMILES: Cn1c(Br)cc2ccccc21 ; CC1(C)OB(C2=CCC(NC(=O)OCc3ccccc3)CC2)OC1(C)C ; Cn1c(C2=CCC(NC(=O)OCc3ccccc3)CC2)cc2ccccc21]

To a 50 mL single-neck flask were added 05 (301 mg, 1.44 mmol), 06 (513 mg, 1.44 mmol), Pd(dppf)Cl₂ (108 mg, 0.14 mmol), K₂CO₃ (602 mg, 4.32 mmol), 1,4-dioxane (8 mL) and water (2 mL), and the mixture was reacted in a nitrogen atmosphere at 100° C. for 16 h, cooled to room temperature, quenched with water (20 mL), and extracted with ethyl acetate (40 mL). The organic phase was washed with saturated brine (20 mL×3) and concentrated to dryness by rotary evaporation, and the residue was purified by column chromatography (ethyl acetate/petroleum ether=1:6) to give the product as a yellow solid (158 mg, 52.5% yield). LC-MS: 361.2 [M+H]⁺.

### Step 6

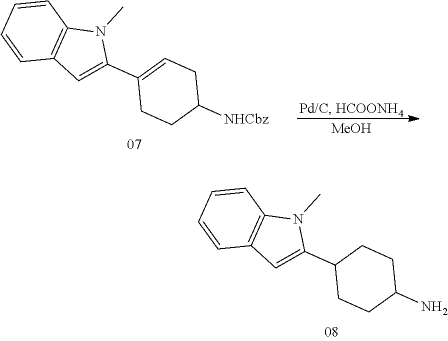 [SMILES: Cn1c(C2=CCC(NC(=O)OCc3ccccc3)CC2)cc2ccccc21 ; Cn1c(C2CCC(N)CC2)cc2ccccc21]

In a 50 mL round-bottom flask, 07 (158 mg, 0.44 mmol) was dissolved in methanol (3 mL), and 10% palladium on carbon (10 mg) and ammonium formate (50 mg) were added. The mixture was reacted at room temperature for 16 h and filtered, and the filtrate was concentrated to dryness by rotary evaporation. The residue was purified by pre-TLC (petroleum ether:ethyl acetate=3:1) to give the target product as a white solid (50 mg, 32% yield). LC-MS: 229.1 [M+H]⁺.

### Step 7

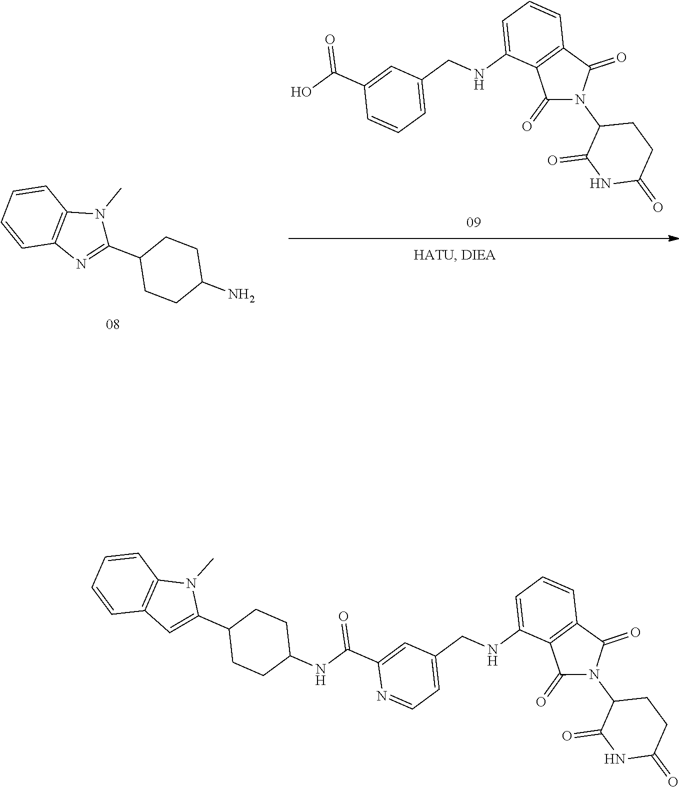 [SMILES: Cn1c(C2CCC(N)CC2)nc2ccccc21 ; O=C1CCC(N2C(=O)c3cccc(NCc4cccc(C(=O)O)c4)c3C2=O)C(=O)N1 ; Cn1c(C2CCC(NC(=O)c3cc(CNc4cccc5c4C(=O)N(C4CCC(=O)NC4=O)C5=O)ccn3)CC2)cc2ccccc21]

In a 50 mL single-neck flask, 08 (45.6 mg, 0.20 mmol) was dissolved in N,N-dimethylformamide (2 mL), and HATU (114 mg, 0.3 mmol), DIEA (77 mg, 0.6 mmol) and 09 (81.4 mg, 0.20 mmol) were added. The mixture was stirred at room temperature for 16 h and purified by preparative reversed-phase liquid chromatography to give product PRJ2-3052 as a yellow solid (2.30 mg, 1.9% yield). LC-MS: 618.2[M+H]^{+ 1}HNMR(400 MHz, DMSO-d₆) δ 11.09 (s, 1H), 8.30-8.15 (m, 1H), 7.86 (d, J=12.5 Hz, 1H), 7.73 (s, 1H), 7.50 (s, 2H), 7.44 (s, 2H), 7.37 (d, J=8.6 Hz, 1H), 7.29 (s, 1H), 7.05 (d, J=7.9 Hz, 2H), 6.96 (d, J=7.7 Hz, 2H), 6.28-6.25 (m, 1H), 5.06 (s, 1H), 4.62 (s, 2H), 3.70 (d, J=4.9 Hz, 3H), 2.88 (s, 2H), 2.65 (d, J=19.4 Hz, 2H), 2.33 (s, 1H), 2.06 (s, 2H), 1.90 (s, 2H), 1.85-1.74 (m, 2H), 1.57 (s, 2H), 1.24 (s, 1H)

### Example 33

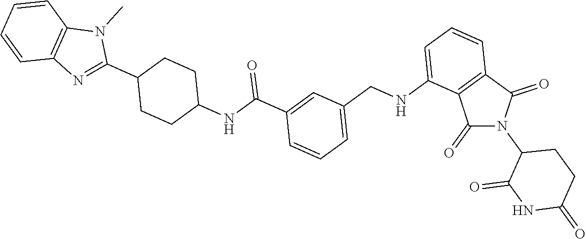 [SMILES: Cn1c(C2CCC(NC(=O)c3cccc(CNc4cccc5c4C(=O)N(C4CCC(=O)NC4=O)C5=O)c3)CC2)nc2ccccc21]

### Preparation Steps

### Step 1

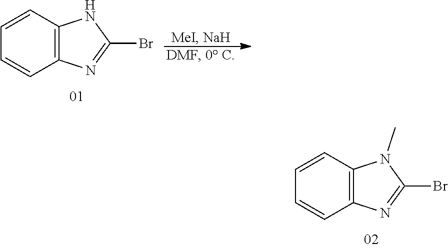

2-Bromo-1H-benzimidazole (500 mg, 2.54 mmol) was dissolved in anhydrous N,N-dimethylformamide (5 mL), and 60% sodium hydrogen (183 mg, 4.57 mmol) was added at 0° C. After the reaction mixture was stirred at 0° C. for 15 min, iodomethane (378 mg, 2.67 mmol) was added. The mixture was stirred at room temperature for 16 h. Water (30 mL) was added, followed by extraction with ethyl acetate (10 mL×3). The organic phase was washed with saturated brine (20 mL), dried over anhydrous sodium sulfate, and filtered, and the filtrate was concentrated to dryness by rotary evaporation. The residue was passed through a reversed-phase column (C18, 5% to 100% acetonitrile/water as mobile phase, 0.1% formic acid) to give the target product as a white solid (250 mg, 46.7% yield). LC-MS: 211.0, 213.0 [M+H]⁺.

### Step 2

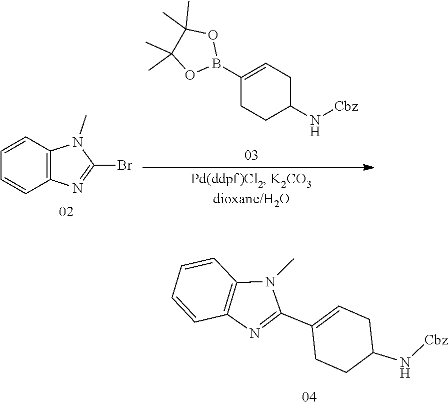

02 (150 mg, 0.710 mmol), 03 (304 mg, 0.850 mmol), potassium carbonate (294 mg, 2.13 mmol) and [1,1′-bis(diphenylphosphino)ferrocene]palladium(II) dichloride (52.0 mg, 0.071 mmol) were mixed in dioxane (5 mL) and water (1 mL). The mixture was purged with argon, then stirred in an argon atmosphere at 90° C. for 16 h, cooled to room temperature, and then concentrated under reduced pressure to remove the solvent. Water (20 mL) was added, followed by extraction with ethyl acetate (10 mL×2). The organic phase was washed with saturated brine (10 mL), dried over anhydrous sodium sulfate, and filtered, and the filtrate was concentrated to dryness by rotary evaporation. The residue was purified through a flash silica gel column (petroleum ether:ethyl acetate=1:1) to give the target product as a white solid (139 mg, 54.1% yield). LC-MS: 362.1 [M+H]⁺.

### Step 3

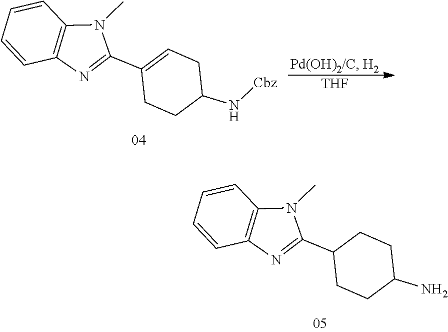 [SMILES: Cn1c(C2=CCC(NC(=O)OCc3ccccc3)CC2)nc2ccccc21 ; Cn1c(C2CCC(N)CC2)nc2ccccc21]

04 (139 mg, 0.385 mmol) was dissolved in tetrahydrofuran (3 mL), followed by addition of 20% palladium hydroxide/carbon (40 mg, 0.285). The mixture was stirred at room temperature for 16 h and filtered, and the filtrate was concentrated to dryness by rotary evaporation to give the target product as a brown oil (75.0 mg, 85.0% yield). LC-MS: 230.2 [M+H]⁺.

### Step 4

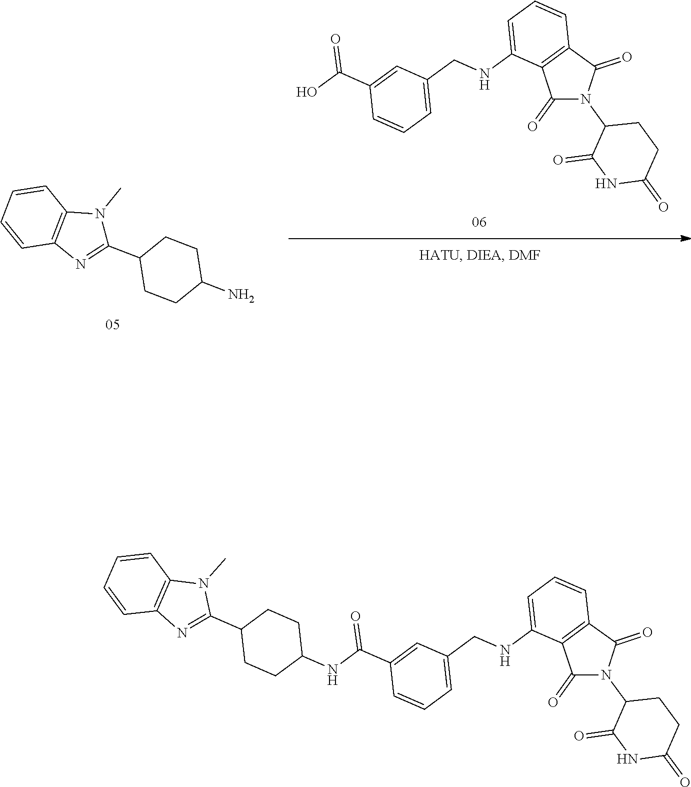 [SMILES: Cn1c(C2CCC(N)CC2)nc2ccccc21 ; O=C1CCC(N2C(=O)c3cccc(NCc4cccc(C(=O)O)c4)c3C2=O)C(=O)N1 ; Cn1c(C2CCC(NC(=O)c3cccc(CNc4cccc5c4C(=O)N(C4CCC(=O)NC4=O)C5=O)c3)CC2)nc2ccccc21]

05 (75.0 mg, 0.327 mmol), 06 (133 mg, 0.327 mmol) and N,N-diisopropylethylamine (127 mg, 0.981 mmol) were mixed in anhydrous N,N-dimethylformamide (2 mL) at room temperature, and (7-azabenzotriazole)-tetramethyluronium hexafluorophosphate (149 mg, 0.392 mmol) was added in batches with stirring at room temperature. The reaction mixture was stirred at room temperature for 2 h. The mixture was purified by preparative chromatography (C18, 5-100% acetonitrile/water as mobile phase, 0.1% formic acid) to separate the target product as a yellow solid (73.7 mg, 36.4% yield). LC-MS: 619.2 [M+H]⁺.

### Example 34

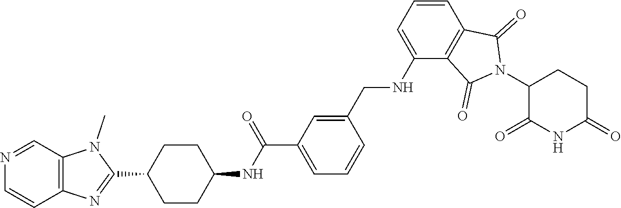 [SMILES: Cn1c2cnccc2nc1[C@H]1CC[C@H](NC(=O)c2cccc(CNc3cccc4c3C(=O)N(C3CCC(=O)NC3=O)C4=O)c2)CC1]

### Preparation Steps

### Step 1

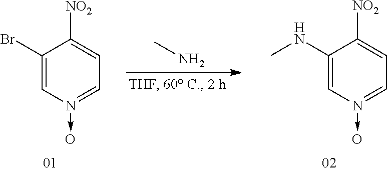

3-Bromo-4-nitropyridine-N-oxide (5.00 g, 22.8 mmol) was dissolved in tetrahydrofuran (150 mL) at room temperature, and 30% methylamine alcohol solution (3.54 g, 114 mmol) was added with stirring at room temperature. The reaction mixture was stirred at 60° C. for 2 h. The mixture was concentrated under reduced pressure to remove the solvent. The crude product was purified through a flash silica gel column (dichloromethane:methanol=10:1) to give the target product as an orange red solid (3.22 g, 83.4% yield). LC-MS: 170.0 [M+H]⁺; 211.0 [M+H⁺41]⁺.

### Step 2

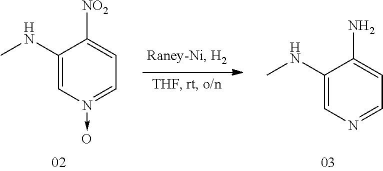

02 (3.22 g, 19.0 mmol) was dissolved in tetrahydrofuran (150 mL) at room temperature, and Raney nickel (1 g) was added. Then the reaction mixture was purged twice with hydrogen and stirred in a hydrogen atmosphere at room temperature for 16 h. The mixture was filtered, and the filtrate was concentrated to dryness by rotary evaporation under reduced pressure to give the target product as a brownish-black solid (2.18 g, 93% yield). LC-MS: 124.1 [M+H]⁺.

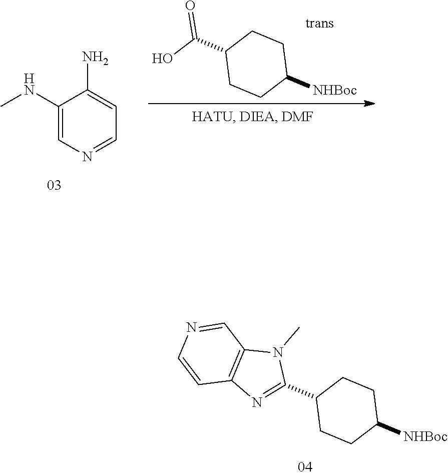

03 (500 mg, 4.06 mmol), trans-4-(Boc-amino)cyclohexanecarboxylic acid (938 mg, 3.86 mmol) and N,N-diisopropylethylamine (1.57 g, 12.2 mmol) were dissolved in anhydrous N,N-dimethylformamide (10 mL), and (7-azabenzotriazole)-tetramethyluronium hexafluorophosphate (2.32 g, 6.09 mmol) was added in batches with stirring at room temperature. The reaction mixture was stirred at room temperature for 2 h. The mixture was purified through a reversed-phase column (C18, 5-100% acetonitrile/water as mobile phase, 0.1% trifluoroacetic acid) to give the target product as a yellow oil (1.20 g, 84.8% yield)(note: unclosed rings in the product were all closed during concentration of the component obtained from the reversed-phase column purification). LC-MS: 331.3 [M+H]⁺; 372.2 [M+H⁺41]⁺; 275.2 [M+H−56]⁺.

### Step 4

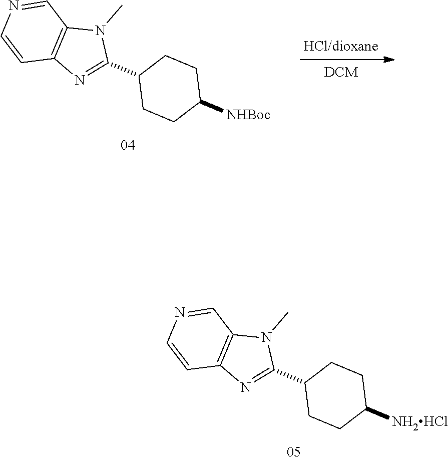

04 (564 mg, 1.71 mmol) was dissolved in anhydrous dichloromethane (2.5 mL), and a solution of 4 M hydrogen chloride in dioxane (2 mL) was added dropwise with stirring at room temperature. The reaction mixture was stirred at room temperature for 1 h. The mixture was concentrated to dryness by rotary evaporation to give the target product as an off-white solid (400 mg, 87.8% yield). LC-MS: 231.1 [M+H]⁺; 272.1 [M+H+41]⁺.

### Step 5

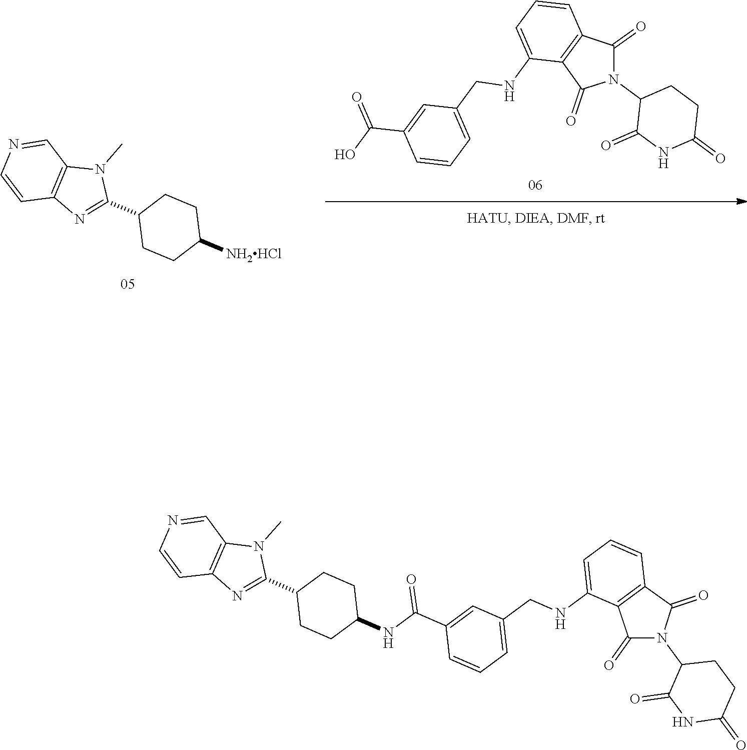

05 (32.8 mg, 0.123 mmol), 06 (50 mg, 0.123 mmol) and N,N-diisopropylethylamine (79.5 mg, 0.615 mmol) were dissolved in anhydrous N,N-dimethylformamide (2 mL), and (7-azabenzotriazole)-tetramethyluronium hexafluorophosphate (56.3 mg, 0.148 mmol) was added in batches with stirring at room temperature. The reaction mixture was stirred at room temperature for 2 h. The mixture was purified by preparative reversed-phase liquid chromatography (C18, 5-100% acetonitrile/water as mobile phase, 0.1% formic acid) to separate the target product as a yellow solid (27 mg, 35.5% yield). LC-MS: 620.4 [M+H]⁺. ¹H NMR (400 MHz, DMSO-d₆) δ 11.11 (s, 1H), 9.36 (s, 1H), 8.54 (d, J=6.4 Hz, 1H), 8.35 (d, J=7.7 Hz, 1H), 8.10 (d, J=6.1 Hz, 1H), 7.86 (s, 1H), 7.74 (d, J=7.8 Hz, 1H), 7.55-7.48 (m, 2H), 7.43 (t, J=7.6 Hz, 1H), 7.30 (t, J=6.3 Hz, 1H), 7.04 (d, J=7.0 Hz, 1H), 6.96 (d, J=8.7 Hz, 1H), 5.08 (dd, J=12.8, 5.4 Hz, 1H), 4.62 (d, J=6.1 Hz, 2H), 4.02 (s, 3H), 3.92-3.83 (m, 1H), 3.19-3.14 (m, 1H), 2.94-2.84 (m, 1H), 2.64-2.54 (m, 2H), 2.13-1.97 (m, 5H), 1.84-1.72 (m, 2H), 1.64-1.53 (m, 2H).

### Example 35

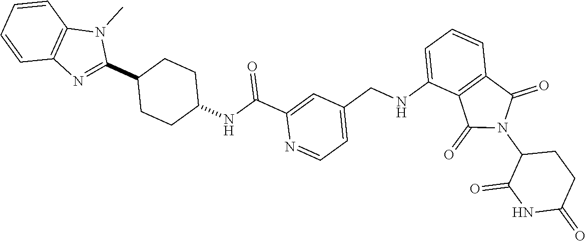

### Preparation Steps

### Step 1

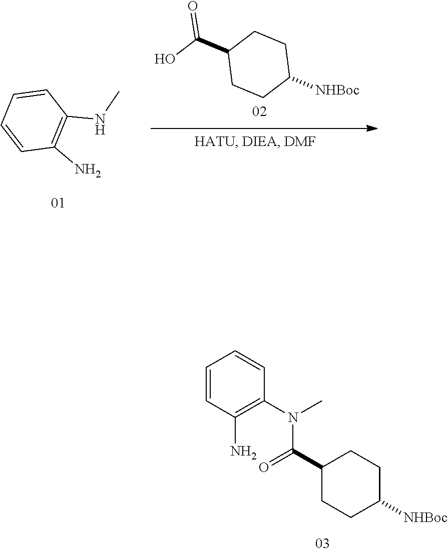

To a 100 mL single-neck flask were added 01 (200 mg, 1.64 mmol), 02 (400 mg, 1.64 mmol), HATU (810 mg, 2.13 mmol), DIEA (634 mg, 4.9 mmol) and DMF (10 mL), and the mixture was reacted at room temperature for 2 h, quenched with water (20 mL), and extracted with ethyl acetate (20 mL×2). The organic phase was washed with saturated brine (20 mL×3) and concentrated to dryness by rotary evaporation, and the residue was purified by column chromatography (dichloromethane:methanol=20:1) to give the target product as a white solid (500 mg, 88% yield). LC-MS: 348.2 [M+H]⁺.

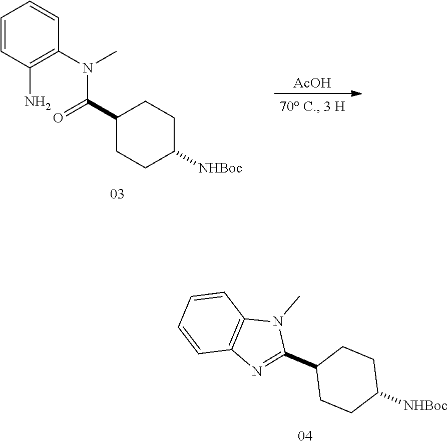

In a 100 mL single-neck round-bottom flask, 03 (250 mg, 0.720 mmol) was dissolved in acetic acid (2 mL), and the mixture was reacted at 70° C. for 3 h and concentrated to dryness by rotary evaporation, and the residue was purified by column chromatography (dichloromethane:methanol=10:1) to give the target product as a pale yellow solid (220 mg, 93% yield). LC-MS: 330.2 [M+H]⁺.

### Step 3

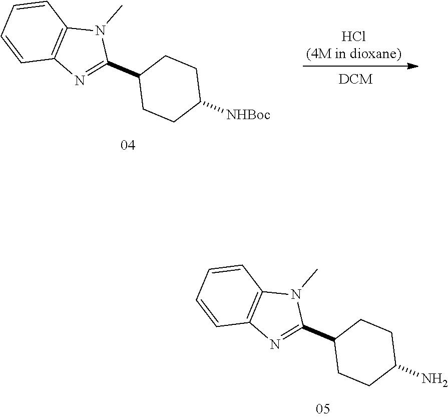 [SMILES: Cn1c2ccccc2nc1[C@H]1CC[C@H](NC(=O)OC(C)(C)C)CC1 ; "HCl(4M in dioxane)" ; Cn1c2ccccc2nc1[C@H]1CC[C@H](N)CC1]

In a 50 mL single-neck flask, 04 (50 mg, 0.152 mmol) was dissolved in dichloromethane (2 mL), and hydrochloric acid in 1,4-dioxane (0.5 mL) was added. The reaction mixture was stirred at room temperature for 1 h and could be directly used in the next step after being concentrated to dryness by rotary evaporation. LC-MS: 230.2 [M+H]⁺.

### Step 4

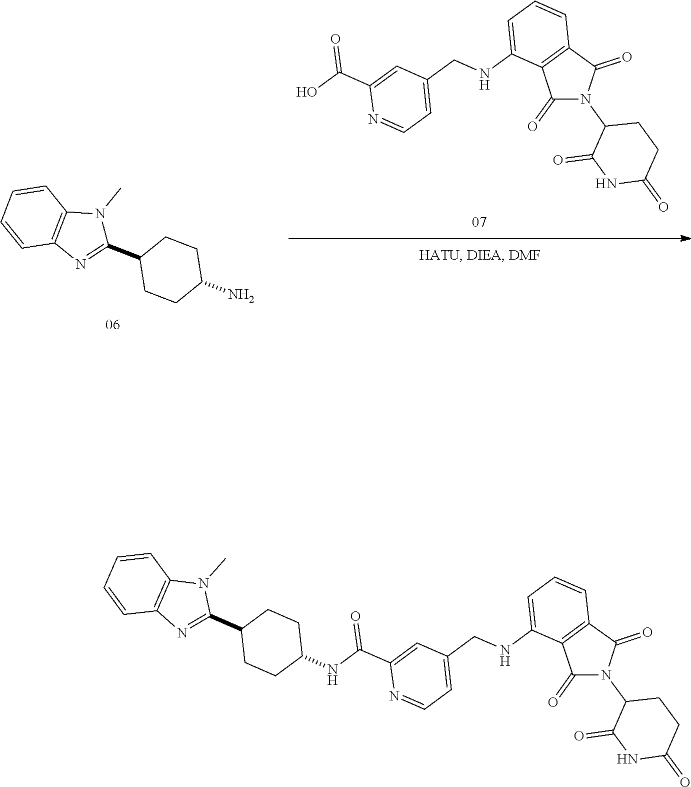 [SMILES: Cn1c2ccccc2nc1[C@H]1CC[C@H](N)CC1 ; O=C1CCC(N2C(=O)c3cccc(NCc4ccnc(C(=O)O)c4)c3C2=O)C(=O)N1 ; Cn1c2ccccc2nc1[C@H]1CC[C@H](NC(=O)c2cc(CNc3cccc4c3C(=O)N(C3CCC(=O)NC3=O)C4=O)ccn2)CC1]

06 (31 mg, 0.135 mmol) was dissolved in N,N-dimethylformamide (1 mL) at room temperature, and 07 (55 mg, 0.135 mmol), HATU (67 mg, 0.176 mmol) and N,N-diisopropylethylamine (87 mg, 0.673 mmol) were added successively. The above mixture was stirred at room temperature for 1 h. The reaction mixture was purified by preparative high performance liquid chromatography to give a yellowish-green solid (30.79 mg, 37% yield over two steps). LC-MS: 620.3 [M+H]⁺, 1H NMR (400 MHz, DMSO-d6) δ 11.11 (s, 1H), 8.70 (d, J=8.4 Hz, 1H), 8.60 (d, J=5.0 Hz, 1H), 8.04 (s, 1H), 7.98 (dd, J=6.3, 2.8 Hz, 1H), 7.81 (dd, J=6.2, 2.9 Hz, 1H), 7.64-7.57 (m, 3H), 7.53-7.46 (m, 2H), 7.06 (d, J=7.1 Hz, 1H), 6.87 (d, J=8.6 Hz, 1H), 5.10 (dd, J=12.8, 5.4 Hz, 1H), 4.72 (d, J=5.5 Hz, 2H), 4.04 (s, 3H), 3.97-3.86 (m, 1H), 3.34 (t, J=12.0 Hz, 1H), 2.98-2.81 (m, 1H), 2.65-2.53 (m, 2H), 2.17 (d, J=11.8 Hz, 2H), 2.13-2.04 (m, 1H), 2.00 (d, J=10.5 Hz, 2H), 1.82 (dd, J=24.2, 11.6 Hz, 2H), 1.70 (dd, J=22.9, 11.8 Hz, 2H).

### Example 36

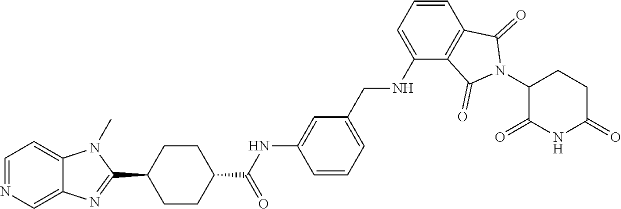 [SMILES: Cn1c2ccncc2nc1[C@H]1CC[C@H](C(=O)Nc2cccc(CNc3cccc4c3C(=O)N(C3CCC(=O)NC3=O)C4=O)c2)CC1]

### Preparation Steps

### Step 1

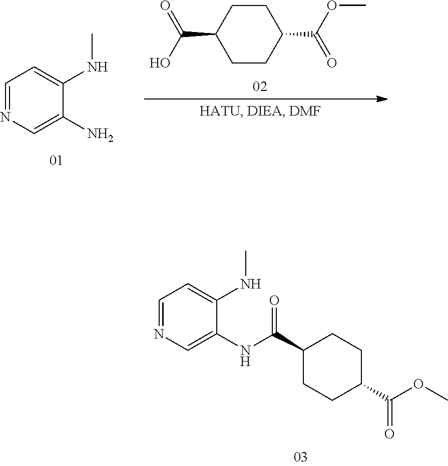

01 (1.00 g, 8.12 mmol), 02 (1.51 g, 8.12 mmol) and N,N-diisopropylethylamine (3.15 mg, 24.4 mmol) were dissolved in anhydrous N,N-dimethylformamide (30 mL), and (7-azabenzotriazole)-tetramethyluronium hexafluorophosphate (4.63 g, 12.2 mmol) was added in batches with stirring at room temperature. The reaction mixture was stirred at room temperature for 16 h. The mixture was passed through a reversed-phase column (C18, 10-100% acetonitrile/water as mobile phase, 0.1% ammonia solution) to separate the target product as a white solid (1.46 g, 61.7% yield). LC-MS: 292.4 [M+H]⁺.

### Step 2

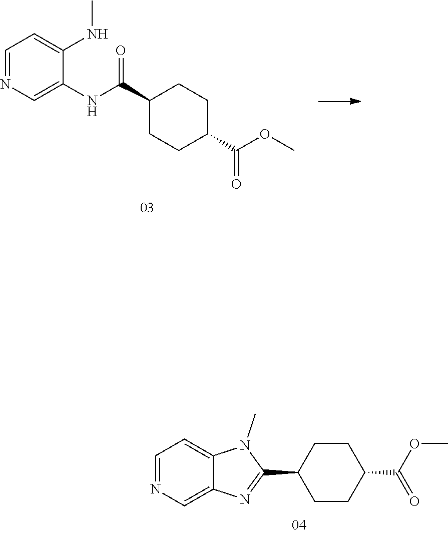 [SMILES: CNc1ccncc1NC(=O)[C@H]1CC[C@H](C(=O)OC)CC1 ; COC(=O)[C@H]1CC[C@H](c2nc3cnccc3n2C)CC1]

03 (200 mg, 0.686 mmol) was added to a sealed tube and dissolved in acetic acid (3 mL). The reaction mixture was stirred at 140° C. for 16 h and concentrated under reduced pressure to remove the solvent to give the target product as a white solid (165 mg, 87.9% yield). LC-MS: 274.3 [M+H]⁺.

### Step 3

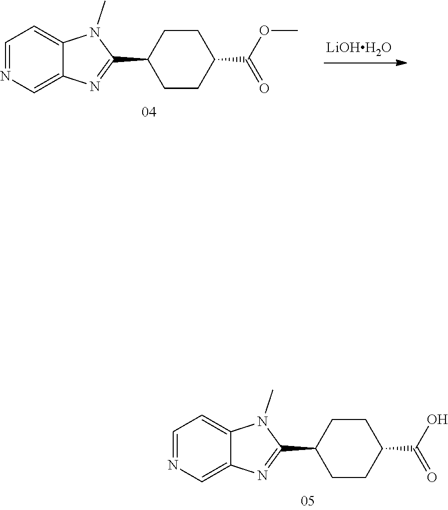

04 (165 mg, 0.604 mmol) was dissolved in tetrahydrofuran (2 mL) and water (0.5 mL) at room temperature, and lithium hydroxide (50.7 mg, 1.21 mmol) was then added. The reaction mixture was stirred at room temperature for 16 h. An appropriate amount of hydrochloric acid (2 M) was added to adjust the pH value of the reaction mixture to 3-4, and then the mixture was concentrated to dryness by rotary evaporation to give the target product as a white solid (165 mg, crude). LC-MS: 260.3 [M+H]⁺.

### Step 4

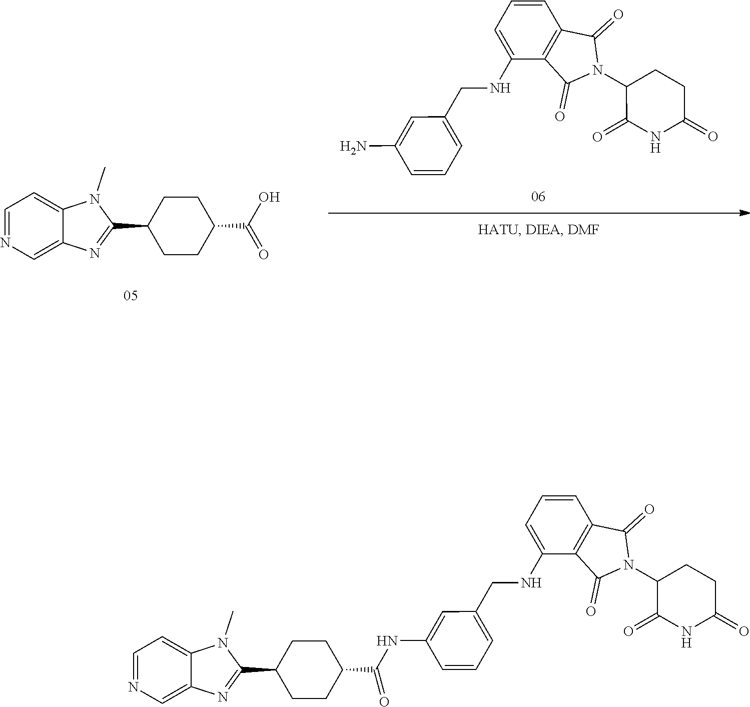 [SMILES: Cn1c2ccncc2nc1[C@H]1CC[C@H](C(=O)O)CC1 ; Nc1cccc(CNc2cccc3c2C(=O)N(C2CCC(=O)NC2=O)C3=O)c1 ; Cn1c2ccncc2nc1[C@H]1CC[C@H](C(=O)Nc2cccc(CNc3cccc4c3C(=O)N(C3CCC(=O)NC3=O)C4=O)c2)CC1]

05 (50.0 mg, 0.193 mmol), 06 (73.0 mg, 0.193 mmol) and N,N-diisopropylethylamine (74.8 mg, 0.579 mmol) were dissolved in anhydrous N,N-dimethylformamide (2 mL), and (7-azabenzotriazole)-tetramethyluronium hexafluorophosphate (110 mg, 0.289 mmol) was added in batches with stirring at room temperature. The reaction mixture was stirred at room temperature for 1 h. The mixture was purified by preparative reversed-phase liquid chromatography (C18, 5-100% acetonitrile/water as mobile phase, 0.1% formic acid) to separate the target product as a yellowish-green solid (12.20 mg, 10.2% yield). LC-MS: 620.4 [M+H]⁺, ¹H NMR (400 MHz, DMSO-d₆) δ 11.10 (s, 1H), 9.92 (s, 1H), 8.83 (s, 1H), 8.29 (d, J=5.5 Hz, 1H), 7.62-7.50 (m, 4H), 7.29-7.21 (m, 2H), 7.04 (d, J=7.0 Hz, 2H), 6.92 (d, J=8.6 Hz, 1H), 5.08 (dd, J=12.8, 5.3 Hz, 1H), 4.54 (d, J=5.9 Hz, 2H), 3.81 (s, 3H), 3.11-3.04 (m, 1H), 2.95-2.85 (m, 1H), 2.62-2.56 (m, 2H), 2.12-2.00 (m, 4H), 1.99-1.91 (m, 2H), 1.73-1.61 (m, 4H).

### Example 37

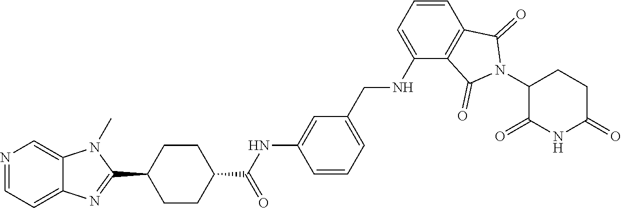 [SMILES: Cn1c2cnccc2nc1[C@H]1CC[C@H](C(=O)Nc2cccc(CNc3cccc4c3C(=O)N(C3CCC(=O)NC3=O)C4=O)c2)CC1]

### Preparation Steps

### Step 1

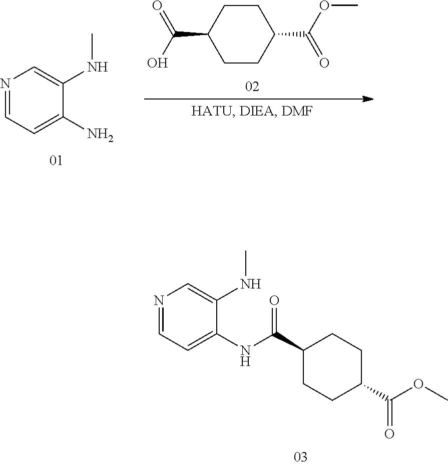

01 (500 mg, 4.06 mmol), 02 (680 mg, 3.15 mmol) and N,N-diisopropylethylamine (1049 mg, 8.12 mmol) were dissolved in N,N-dimethylformamide (8 mL) at room temperature, and (7-azabenzotriazole)-tetramethyluronium hexafluorophosphate (1852 mg, 4.87 mmol) was added with stirring at room temperature. The reaction mixture was stirred at room temperature for 2 h and passed through a reversed-phase column (C18, water:acetonitrile=100:0 to 35:65, 0.1% formic acid) to separate the target product as a white solid (190 mg, 20.7% yield). LC-MS: 292.4 [M+H]⁺.

### Step 2

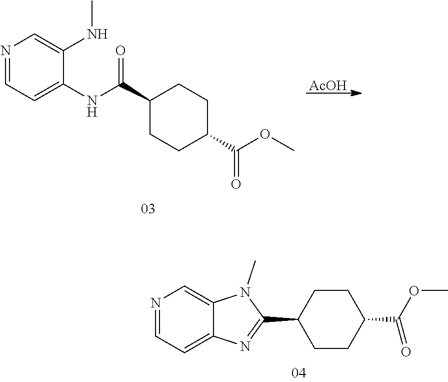 [SMILES: "AcOH" ; CNc1cnccc1NC(=O)[C@H]1CC[C@H](C(=O)OC)CC1 ; COC(=O)[C@H]1CC[C@H](c2nc3ccncc3n2C)CC1]

03 (190 mg, 0.65 mmol) was dissolved in acetic acid (2 mL) at room temperature, and the reaction mixture was stirred in an argon atmosphere at 70° C. for 2 h and concentrated to dryness by rotary evaporation to give the target product as a yellow oil (140 mg, 78.9% yield). LC-MS: 274.2 [M+H]⁺.

### Step 3

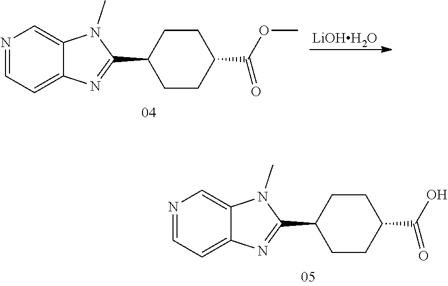

04 (140 mg, 0.51 mmol) was dissolved in tetrahydrofuran (2 mL) and water (2 mL) at room temperature, and lithium hydroxide monohydrate (52 mg, 1.23 mmol) was added at room temperature. The reaction mixture was stirred at room temperature for 3 h and concentrated to dryness by rotary evaporation. Water (20 mL) and ethyl acetate (20 mL) were added, and the aqueous phase was separated, adjusted to a pH value less than 7, and extracted with ethyl acetate (30 mL). The organic phase was concentrated to dryness by rotary evaporation to give the target product as a white solid (90 mg, 68.1% yield). LC-MS: 260.2 [M+H]⁺.

### Step 4

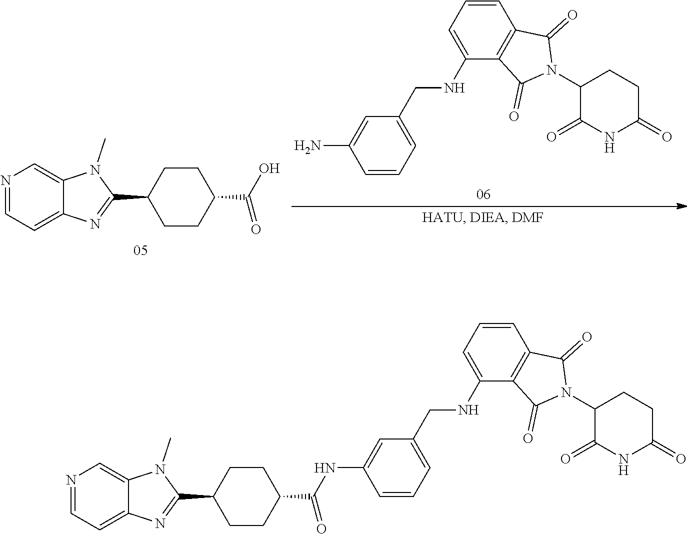 [SMILES: Cn1c2cnccc2nc1[C@H]1CC[C@H](C(=O)O)CC1 ; Nc1cccc(CNc2cccc3c2C(=O)N(C2CCC(=O)NC2=O)C3=O)c1 ; Cn1c2cnccc2nc1[C@H]1CC[C@H](C(=O)Nc2cccc(CNc3cccc4c3C(=O)N(C3CCC(=O)NC3=O)C4=O)c2)CC1]

05 (90 mg, 0.35 mmol), 06 (133 mg, 0.35 mmol) and N,N-diisopropylethylamine (136 mg, 1.05 mmol) were dissolved in N,N-dimethylformamide (1 mL) at room temperature, and (7-azabenzotriazole)-tetramethyluronium hexafluorophosphate (160 mg, 0.42 mmol) was added with stirring at room temperature. The reaction mixture was stirred at room temperature for 2 h. The mixture was purified by preparative chromatography (C18, 5-100% acetonitrile/water as mobile phase, 0.1% formic acid) to separate the target product as a yellow solid (1.83 mg, 0.8% yield). LC-MS: 620.5 [M+H]⁺.

¹H NMR (400 MHz, DMSO-d₆) δ 11.11 (s, 1H), 9.93 (s, 1H), 8.87 (s, 1H), 8.28 (d, J=5.5 Hz, 1H), 7.61 (d, J=9.3 Hz, 1H), 7.58-7.49 (m, 3H), 7.30-7.21 (m, 2H), 7.04 (d, J=7.0 Hz, 2H), 6.92 (d, J=8.7 Hz, 1H), 5.08 (dd, J=13.0, 5.5 Hz, 1H), 4.54 (d, J=5.8 Hz, 2H), 3.88 (s, 3H), 3.08 (s, 1H), 2.96-2.84 (m, 1H), 2.65 (d, J=19.2 Hz, 2H), 2.07-1.93 (m, 6H), 1.67 (t, J=9.8 Hz, 4H).

### Example 38

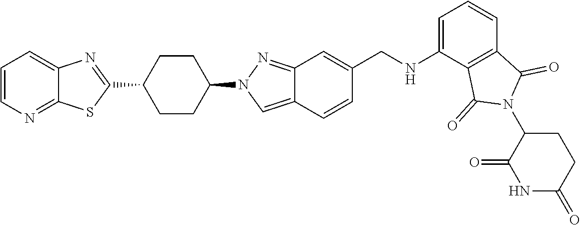 [SMILES: O=C1CCC(N2C(=O)c3cccc(NCc4ccc5cn([C@H]6CC[C@H](c7nc8cccnc8s7)CC6)nc5c4)c3C2=O)C(=O)N1]

### Preparation Steps

### Step 1

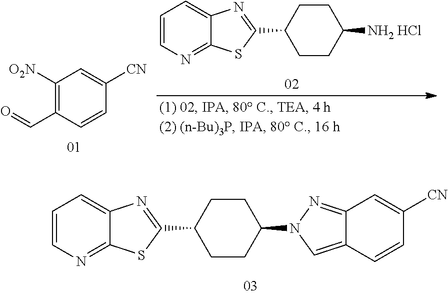

01 (65 mg, 0.369 mmol), 02 (100 mg, 0.370 mmol) and triethylamine (75 mg, 0.742 mmol) were dissolved in isopropanol (10 mL) at room temperature, and the reaction mixture was stirred in a nitrogen atmosphere at 80° C. for 4 h and then cooled to room temperature. A solution of tri-n-butylphosphine (225 mg, 1.11 mmol) in isopropanol (1 mL) was then added at room temperature, and the reaction mixture was stirred at 80° C. for 16 h, cooled to room temperature, and filtered. The filter cake was washed with isopropanol and dried to give the target product as a pale yellow solid (100 mg, 75.5% yield). LC-MS: 360.1 [M+H]⁺.

### Step 2

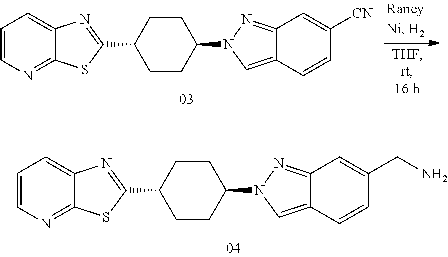 [SMILES: N#Cc1ccc2cn([C@H]3CC[C@H](c4nc5cccnc5s4)CC3)nc2c1 ; NCc1ccc2cn([C@H]3CC[C@H](c4nc5cccnc5s4)CC3)nc2c1]

03 (30 mg, 0.0836 mmol) was dissolved in tetrahydrofuran (5 mL) at room temperature, and Raney nickel (50 mg) was added at room temperature. The reaction mixture was purged with hydrogen three times, stirred in a hydrogen atmosphere at room temperature for 16 h, and filtered, and the filtrate was concentrated to dryness by rotary evaporation to give the target product as a white solid (20 mg, 65.9% yield). LC-MS: 364.0 [M+H]⁺.

### Step 3

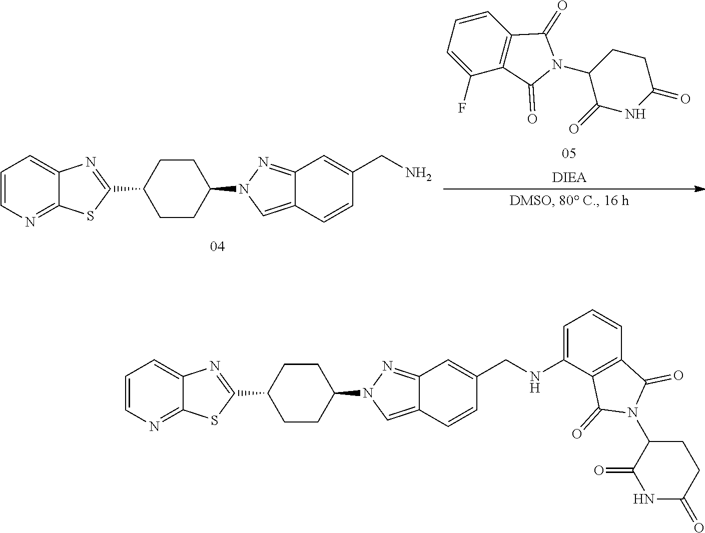 [SMILES: O=C1CCC(N2C(=O)c3cccc(F)c3C2=O)C(=O)N1 ; NCc1ccc2cn([C@H]3CC[C@H](c4nc5cccnc5s4)CC3)nc2c1 ; O=C1CCC(N2C(=O)c3cccc(NCc4ccc5cn([C@H]6CC[C@H](c7nc8cccnc8s7)CC6)nc5c4)c3C2=O)C(=O)N1]

04 (20 mg, 0.0551 mmol), 05 (15 mg, 0.0543 mmol) and N,N-diisopropylethylamine (21 mg, 0.163 mmol) were dissolved in anhydrous dimethyl sulfoxide (1 mL) at room temperature, and the reaction mixture was stirred at 80° C. for 16 h, cooled to 20° C., and purified by preparative chromatography (C18, 5-100% acetonitrile/water as mobile phase, 0.1% formic acid) to separate the target product as a yellow solid (3.08 mg, 9.03% yield). LC-MS: 620.2 [M+H]⁺. ¹H NMR (400 MHz, DMSO-d₆) δ 11.11 (s, 1H), 8.60 (d, J=4.6 Hz, 1H), 8.39 (s, 1H), 8.37 (d, J=8.2 Hz, 1H), 7.68 (d, J=8.6 Hz, 1H), 7.61-7.57 (m, 1H), 7.56 (s, 1H), 7.54-7.47 (m, 1H), 7.28 (t, J=6.0 Hz, 1H), 7.06 (t, J=9.3 Hz, 2H), 7.02 (d, J=7.1 Hz, 1H), 5.08 (dd, J=12.8, 5.4 Hz, 1H), 4.64 (d, J=6.2 Hz, 3H), 2.89 (dd, J=21.6, 9.9 Hz, 1H), 2.61 (dd, J=29.5, 12.5 Hz, 3H), 2.36 (d, J=13.3 Hz, 2H), 2.27 (d, J=9.6 Hz, 2H), 2.15 (dd, J=24.8, 12.5 Hz, 2H), 2.09-1.97 (m, 1H), 1.97-1.83 (m, 2H).

### Example 39

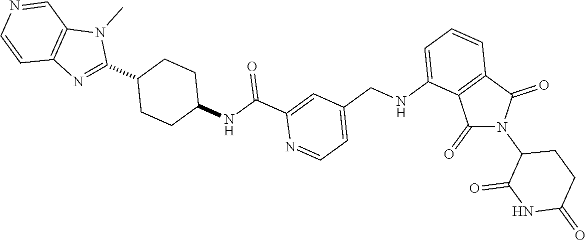

### Preparation Steps

### Step 1

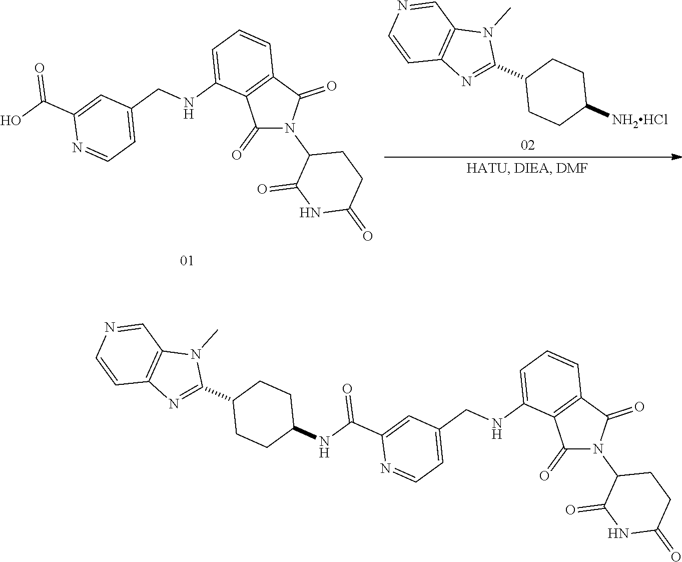

01 (465 mg, 1.14 mmol), 02 (263 mg, 1.14 mmol) and N,N-diisopropylethylamine (442 mg, 3.42 mmol) were dissolved in anhydrous N,N-dimethylformamide (15 mL), and (7-azabenzotriazole)-tetramethyluronium hexafluorophosphate (650 mg, 1.71 mmol) was added in batches with stirring at room temperature. The reaction mixture was stirred at room temperature for 2 h. The mixture was passed through a reversed-phase column (C18, 5-100% acetonitrile/water as mobile phase, 0.1% formic acid) and a silica gel column (methanol/dichloromethane=1:10) to separate the target product as a yellow solid (330 mg, 46.7% yield). LC-MS: 621.4 [M+H]⁺. ¹H NMR (400 MHz, DMSO-d₆) δ 11.12 (s, 1H), 9.09 (s, 1H), 8.66-8.52 (m, 2H), 8.39 (d, J=5.7 Hz, 1H), 8.04 (s, 1H), 7.79 (d, J=5.9 Hz, 1H), 7.57 (d, J=3.7 Hz, 1H), 7.53-7.43 (m, 2H), 7.05 (d, J=7.0 Hz, 1H), 6.87 (d, J=8.6 Hz, 1H), 5.10 (dd, J=12.8, 5.4 Hz, 1H), 4.71 (d, J=6.3 Hz, 2H), 3.94 (s, 3H), 3.91-3.83 (m, 1H), 3.11-3.03 (m, 1H), 2.96-2.85 (m, 1H), 2.64-2.52 (m, 2H), 2.12-1.94 (m, 5H), 1.83-1.61 (m, 4H).

### Example 40

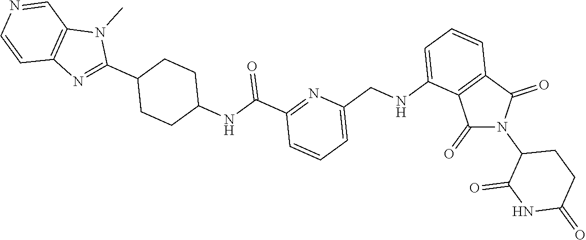 [SMILES: Cn1c(C2CCC(NC(=O)c3cccc(CNc4cccc5c4C(=O)N(C4CCC(=O)NC4=O)C5=O)n3)CC2)nc2ccncc21]

### Preparation Steps

### Step 1

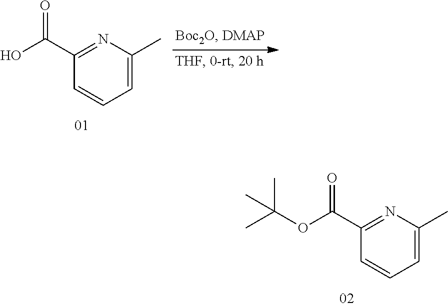

6-Methyl-2-pyridinecarboxylic acid (500 mg, 3.65 mmol) and 4-dimethylaminopyridine (223 mg, 1.82 mmol) were dissolved in anhydrous tetrahydrofuran (10 mL), and di-tert-butyl dicarbonate (1.03 g, 4.74 mmol) was added dropwise at 0° C. The reaction mixture was stirred at room temperature for 20 h. The mixture was concentrated under reduced pressure to remove the solvent. Water (30 mL) was added, followed by extraction with ethyl acetate (10 mL×2). The organic phase was washed with saturated brine (10 mL), dried over anhydrous sodium sulfate, and filtered, and the filtrate was concentrated to dryness by rotary evaporation. The residue was purified through a flash silica gel column (petroleum ether:ethyl acetate=10:1) to give the target product as a colorless transparent oil (570 mg, 80.9% yield). LC-MS: 194.2 [M+H]⁺; 138.2 [M+H]⁺.

### Step 2

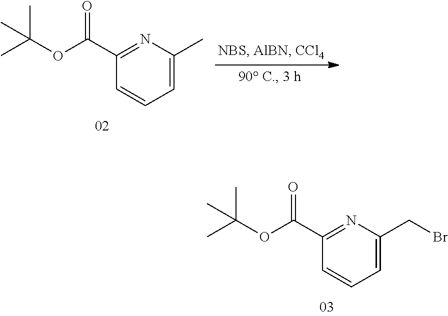

02 (570 mg, 2.95 mmol), N-bromosuccinimide (577 mg, 3.24 mmol) and azobisisobutyronitrile (48.4 mg, 0.295 mmol) were mixed in carbon tetrachloride (5 mL), and the reaction mixture was stirred at 90° C. for 3 h, cooled to room temperature, and concentrated under reduced pressure to remove the solvent. The residue was purified through a flash silica gel column (petroleum ether:ethyl acetate=10:1) to give the target product as a white solid (390 mg, 48.6% yield). LC-MS: 272.2, 274.2 [M+H]⁺; 216.1, 218.1 [M+H−56]⁺.

### Step 3

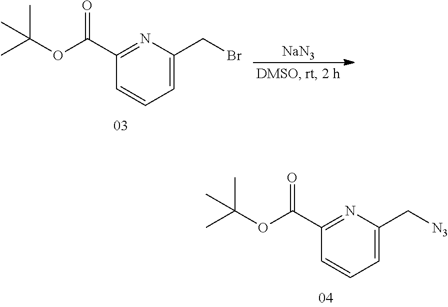 [SMILES: CC(C)(C)OC(=O)c1cccc(CBr)n1 ; CC(C)(C)OC(=O)c1cccc(CN=[N+]=[N-])n1 ; "NaN3"]

03 (390 mg, 1.43 mmol) was dissolved in anhydrous dimethyl sulfoxide (5 mL), and sodium azide (140 mg, 2.15 mmol) was added in batches with stirring at room temperature. The reaction mixture was stirred at room temperature for 2 h. The mixture was poured into water (50 mL), followed by extraction with ethyl acetate (10 mL×2). The organic phase was washed with saturated brine (20 mL), dried over anhydrous sodium sulfate, and filtered, and the filtrate was concentrated to dryness by rotary evaporation. The residue was purified through a flash silica gel column (petroleum ether:ethyl acetate=10:1) to give the target product as a yellow oil (310 mg, 92.3% yield). LC-MS: 235.3 [M+H]⁺; 179.2 [M+H−56]⁺.

### Step 4

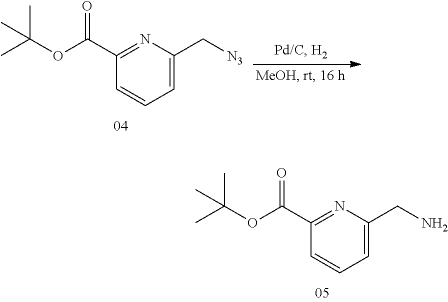 [SMILES: CC(C)(C)OC(=O)c1cccc(CN=[N+]=[N-])n1 ; CC(C)(C)OC(=O)c1cccc(CN)n1]

05 (160 mg, 0.683 mmol) was dissolved in methanol (5 mL), and wet palladium on carbon (100 mg, containing 10% palladium and 50% water) was added. The reaction mixture was purged with hydrogen, then stirred in a hydrogen atmosphere at room temperature for 16 h, and filtered, and the filtrate was concentrated to dryness by rotary evaporation to give the target product as a yellow solid (116 mg, 81.6% yield). LC-MS: 209.3 [M+H]⁺; 153.3 [M+H−56]⁺.

### Step 5

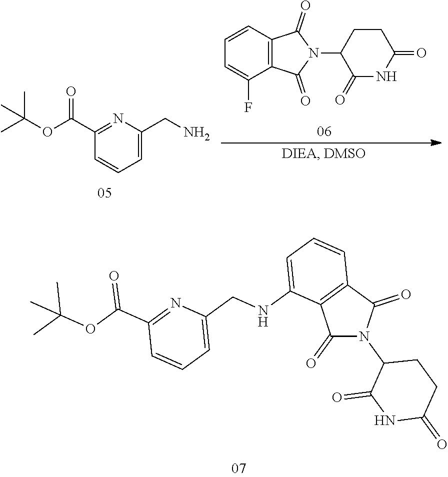 [SMILES: CC(C)(C)OC(=O)c1cccc(CN)n1 ; O=C1CCC(N2C(=O)c3cccc(F)c3C2=O)C(=O)N1 ; CC(C)(C)OC(=O)c1cccc(CNc2cccc3c2C(=O)N(C2CCC(=O)NC2=O)C3=O)n1]

05 (110 mg, 0.528 mmol), 2-(2,6-dioxo-piperidin-3-yl)-4-fluoro-isoindole-1,3-dione (146 mg, 0.528 mmol) and N,N-diisopropylethylamine (273 mg, 2.11 mmol) were dissolved in dimethyl sulfoxide (2 mL), and the reaction mixture was stirred at 120° C. for 5 h, cooled to room temperature, and then purified through a reversed-phase column (C18, 10-100% acetonitrile/water as mobile phase, 0.1% formic acid) to give the target product as a yellow solid (90.0 mg, 36.7% yield). LC-MS: 465.4 [M+H]⁺; 409.3 [M+H−56]⁺.

### Step 6

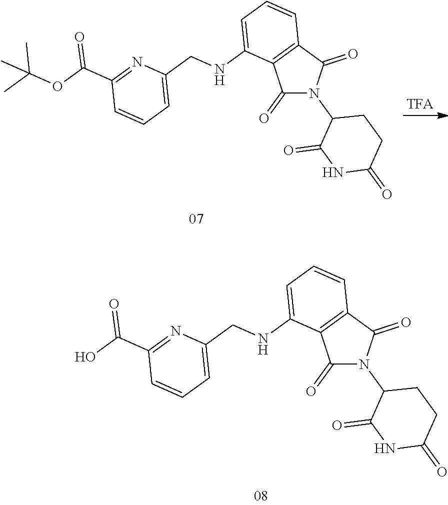 [SMILES: CC(C)(C)OC(=O)c1cccc(CNc2cccc3c2C(=O)N(C2CCC(=O)NC2=O)C3=O)n1 ; O=C1CCC(N2C(=O)c3cccc(NCc4cccc(C(=O)O)n4)c3C2=O)C(=O)N1]

07 (50 mg, 0.108 mmol) was dissolved in dichloromethane (2.5 mL), and trifluoroacetic acid (0.5 mL) was added dropwise with stirring at room temperature. The reaction mixture was stirred at room temperature for 14 h and concentrated under reduced pressure to remove the solvent to give the target product as a yellow solid (40.0 mg, 98.0% yield). LC-MS: 409.3 [M+H]⁺; 431.3 [M+Na]⁺.

### Step 7

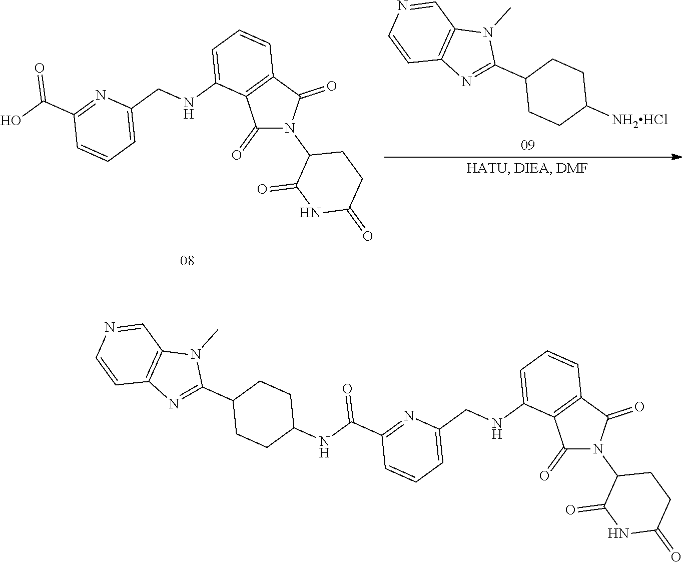

08 (40.0 mg, 0.0980 mmol), 09 (26.1 mg, 0.0980 mmol) and N,N-diisopropylethylamine (50.7 mg, 0.392 mmol) were dissolved in anhydrous N,N-dimethylformamide (1 mL), and (7-azabenzotriazole)-tetramethyluronium hexafluorophosphate (44.9 mg, 0.118 mmol) was added in batches with stirring at room temperature. The reaction mixture was stirred at room temperature for 2 h. The mixture was purified by preparative chromatography (C18, 5-100% acetonitrile/water as mobile phase, 0.1% formic acid) to separate the target product as a yellow solid (13.06 mg, 31.2% yield).

LC-MS: 621.4 [M+H]⁺. ¹H NMR (400 MHz, DMSO-d₆) δ 11.18 (s, 1H), 8.94 (s, 1H), 8.31 (d, J=5.6 Hz, 1H), 8.18 (d, J=8.1 Hz, 1H), 8.04-7.97 (m, 2H), 7.76 (t, J=5.6 Hz, 1H), 7.65-7.59 (m, 3H), 7.14 (s, 1H), 7.12 (s, 1H), 5.14 (dd, J=12.7, 5.2 Hz, 1H), 4.78 (d, J=5.2 Hz, 2H), 3.92 (s, 3H), 3.91-3.86 (m, 1H), 3.00-2.88 (m, 2H), 2.65-2.57 (m, 2H), 2.12-2.00 (m, 5H), 1.83-1.74 (m, 2H), 1.59-1.50 (m, 2H).

### Example 41

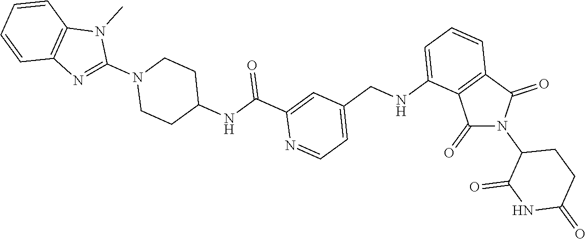 [SMILES: Cn1c(N2CCC(NC(=O)c3cc(CNc4cccc5c4C(=O)N(C4CCC(=O)NC4=O)C5=O)ccn3)CC2)nc2ccccc21]

### Preparation Steps

### Step 1

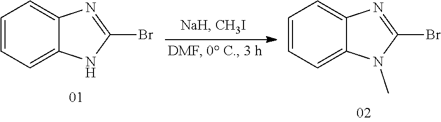

Sodium hydride (153 mg, 3.82 mmol, 60%) was added to a 100 mL three-necked flask and cooled to 0° C. in an argon atmosphere. 01 (500 mg, 2.538 mmol) was dissolved in DMF (5 mL), and the mixture was added to the reaction system. After the reaction system was reacted at 0° C. for 0.5 h, iodomethane (432 mg, 3.04 mmol) was added. The reaction system was reacted at 0° C. for 3 h, quenched with water (50 mL), and extracted with ethyl acetate (30 mL×3). The organic phases were combined, washed with saturated brine (30 mL×2), dried over anhydrous sodium sulfate, and concentrated to dryness by rotary evaporation, and the residue was purified by column chromatography (petroleum ether:ethyl acetate=3:1) to give the target product as a white solid (420 mg, 78.4% yield). LC-MS: 211.0/213.0 [M+H]⁺.

### Step 2

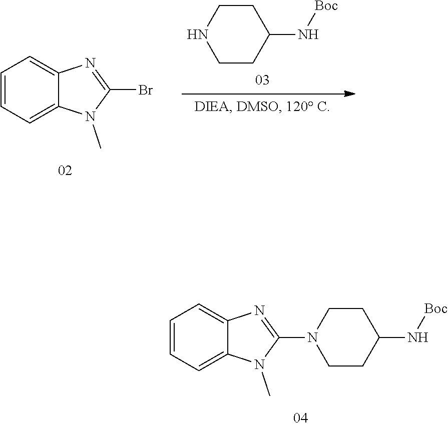 [SMILES: Cn1c(Br)nc2ccccc21 ; CC(C)(C)OC(=O)NC1CCNCC1 ; Cn1c(N2CCC(NC(=O)OC(C)(C)C)CC2)nc2ccccc21]

To a 100 mL single-neck round-bottom flask were added 02 (100 mg, 0.474 mmol), 03 (95 mg, 0.474 mmol), DIEA (306 mg, 2.31 mmol) and DMSO (10 mL), and the mixture was reacted in a nitrogen atmosphere at 120° C. for 16 h, diluted with water (20 mL), and extracted with ethyl acetate (20 mL×3). The organic phases were combined, washed with saturated brine (20 mL×2), dried over anhydrous sodium sulfate, and concentrated to dryness by rotary evaporation, and the residue was purified by column chromatography (petroleum ether:ethyl acetate=1:1) to give the target product as a brown solid (130 mg, 83.0% yield). LC-MS: 331.4 [M+H]⁺.

### Step 3

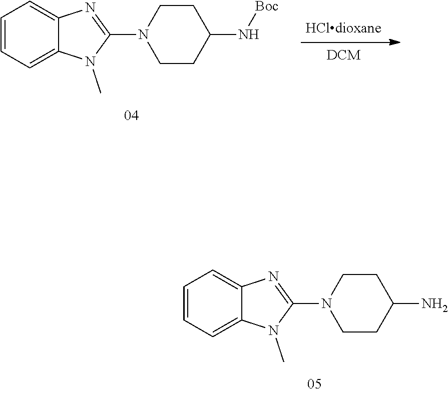 [SMILES: Cn1c(N2CCC(NC(=O)OC(C)(C)C)CC2)nc2ccccc21 ; Cn1c(N2CCC(N)CC2)nc2ccccc21]

To a 100 mL single-neck flask were added 04 (130 mg, 0.393 mmol), hydrochloric acid (4 M, 2 mL) and dichloromethane (5 mL), and the mixture was reacted at room temperature for 2 h and concentrated to give a white solid, which could be directly used in the next step. LC-MS: 231.2 [M+H]⁺.

### Step 4

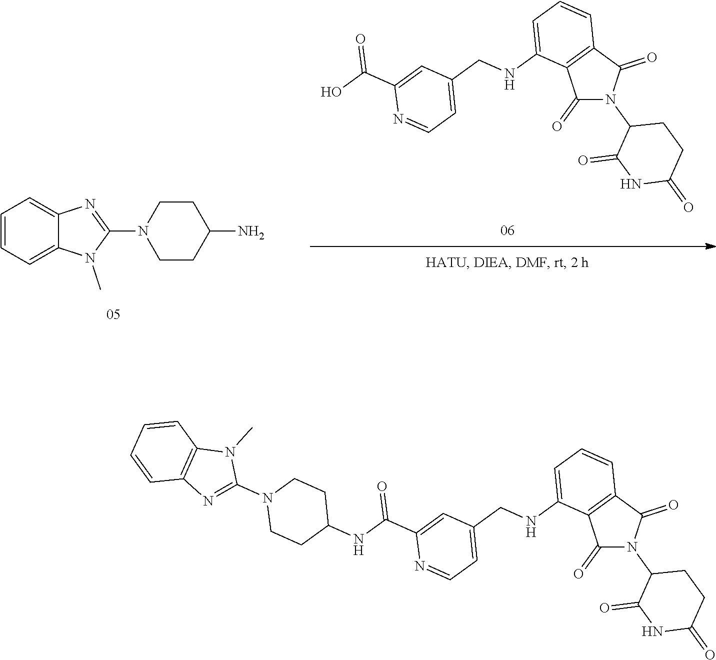 [SMILES: Cn1c(N2CCC(N)CC2)nc2ccccc21 ; O=C1CCC(N2C(=O)c3cccc(NCc4ccnc(C(=O)O)c4)c3C2=O)C(=O)N1 ; Cn1c(N2CCC(NC(=O)c3cc(CNc4cccc5c4C(=O)N(C4CCC(=O)NC4=O)C5=O)ccn3)CC2)nc2ccccc21]

To a 100 mL single-neck flask were added 05 (20 mg, 0.087 mmol), 06 (36 mg, 0.088 mmol), HATU (50 mg, 0.131 mmol), DIEA (57 mg, 0.44 mmol) and DMF (1 mL), and the mixture was reacted at room temperature for 2 h and purified by preparative high performance liquid chromatography to give the target product as a yellow solid (14.46 mg, 26.8% yield). LC-MS: 621.3 [M+H]⁺. ¹H NMR (400 MHz, DMSO-d₆) δ 11.12 (s, 1H), 8.79 (d, J=8.2 Hz, 1H), 8.59 (d, J=4.9 Hz, 1H), 8.06 (s, 1H), 7.67 (d, J=8.5 Hz, 1H), 7.59 (d, J=4.2 Hz, 1H), 7.55-7.45 (m, 3H), 7.45-7.36 (m, 2H), 7.06 (d, J=7.0 Hz, 1H), 6.87 (d, J=8.6 Hz, 1H), 5.10 (dd, J=12.9, 5.3 Hz, 1H), 4.71 (d, J=5.5 Hz, 2H), 4.16 (s, 1H), 3.92 (d, J=12.7 Hz, 2H), 3.76 (s, 3H), 3.46 (t, J=10.5 Hz, 2H), 2.91 (t, J=13.0 Hz, 1H), 2.68-2.52 (m, 2H), 1.95 (t, J=21.9 Hz, 5H).

### Example 42

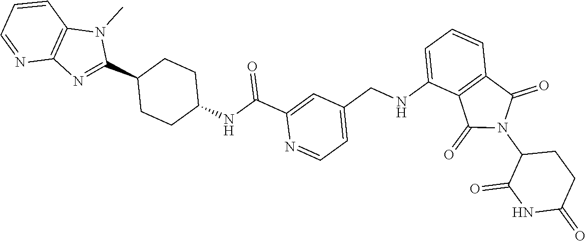 [SMILES: Cn1c2cccnc2nc1[C@H]1CC[C@H](NC(=O)c2cc(CNc3cccc4c3C(=O)N(C3CCC(=O)NC3=O)C4=O)ccn2)CC1]

### Preparation Steps

### Step 1

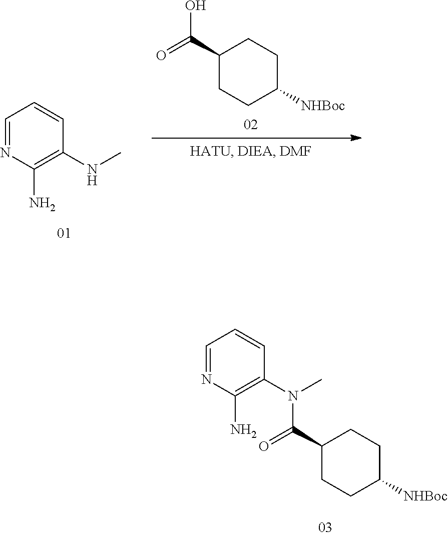

01 (100 mg, 0.812 mmol), 02 (198 mg, 0.812 mmol) and N,N-diisopropylethylamine (315 mg, 2.44 mmol) were dissolved in anhydrous N,N-dimethylformamide (3 mL), and (7-azabenzotriazole)-tetramethyluronium hexafluorophosphate (464 mg, 1.22 mmol) was added in batches with stirring at room temperature. The reaction mixture was stirred at room temperature for 16 h. The mixture was passed through a reversed-phase column (C18, 10-100% acetonitrile/water as mobile phase, 0.03% trifluoroacetic acid) to separate the target product as a brown oil (260 mg, 91.8% yield). LC-MS: 349.4 [M+H]⁺.

### Step 2

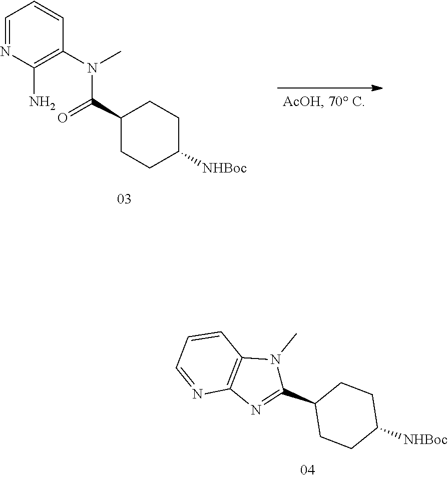 [SMILES: CN(c1cccnc1N)C(=O)[C@H]1CC[C@H](NC(=O)OC(C)(C)C)CC1 ; Cn1c2cccnc2nc1[C@H]1CC[C@H](NC(=O)OC(C)(C)C)CC1]

03 (260 mg, 0.746 mmol) was dissolved in acetic acid (2 mL), and the reaction mixture was stirred at 70° C. for 3 h and concentrated under reduced pressure to remove the solvent to give the target product as a brown oil (246 mg, crude). LC-MS: 331.4 [M+H]⁺.

### Step 3

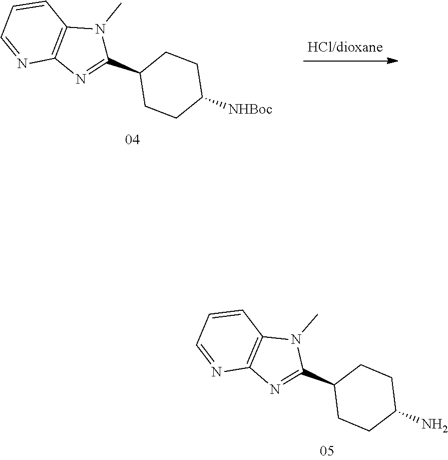 [SMILES: Cn1c2cccnc2nc1[C@H]1CC[C@H](NC(=O)OC(C)(C)C)CC1 ; Cn1c2cccnc2nc1[C@H]1CC[C@H](N)CC1]

04 (246 mg, 0.744 mmol) was dissolved in dichloromethane (2 mL) and methanol (1 mL), and a solution of hydrogen chloride in dioxane (2 mL) was added with stirring at room temperature. The reaction mixture was stirred at room temperature for 1 h and concentrated under reduced pressure to remove the solvent to give the target product as a white solid (131 mg, 76.2% yield over two steps). LC-MS: 231.3 [M+H]⁺.

### Step 4

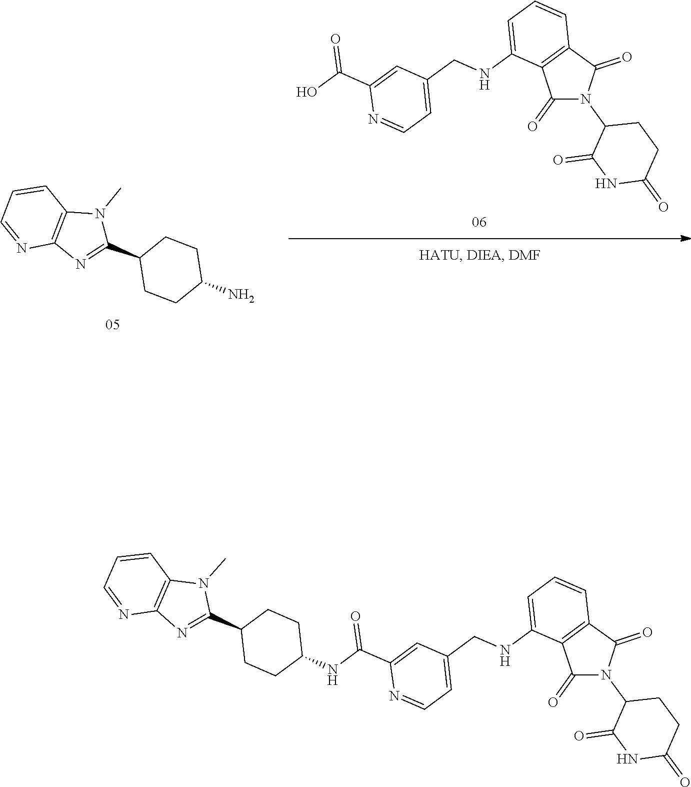 [SMILES: Cn1c2cccnc2nc1[C@H]1CC[C@H](N)CC1 ; O=C1CCC(N2C(=O)c3cccc(NCc4ccnc(C(=O)O)c4)c3C2=O)C(=O)N1 ; Cn1c2cccnc2nc1[C@H]1CC[C@H](NC(=O)c2cc(CNc3cccc4c3C(=O)N(C3CCC(=O)NC3=O)C4=O)ccn2)CC1]

05 (22.6 mg, 0.098 mmol), 06 (40.0 mg, 0.098 mmol) and N,N-diisopropylethylamine (38.0 mg, 0.294 mmol) were dissolved in anhydrous N,N-dimethylformamide (1 mL), and (7-azabenzotriazole)-tetramethyluronium hexafluorophosphate (55.9 mg, 0.147 mmol) was added in batches with stirring at room temperature. The reaction mixture was stirred at room temperature for 16 h. The mixture was purified by preparative reversed-phase liquid chromatography (C18, 5-100% acetonitrile/water as mobile phase, 0.1% formic acid) to separate the target product as a yellowish-green solid (13.3 mg, 21.6% yield). LC-MS: 621.3 [M+H]⁺, 1H NMR (400 MHz, DMSO) δ 11.12 (s, 1H), 8.64-8.56 (m, 2H), 8.39 (s, 1H), 8.13 (s, 1H), 8.09-8.00 (m, 2H), 7.57 (d, J=4.7 Hz, 1H), 7.54-7.45 (m, 2H), 7.28 (dd, J=8.2, 4.6 Hz, 1H), 7.05 (d, J=7.0 Hz, 1H), 6.87 (d, J=8.6 Hz, 1H), 5.10 (dd, J=12.9, 5.3 Hz, 1H), 4.71 (d, J=6.4 Hz, 2H), 3.94-3.81 (m, 4H), 3.08-3.00 (m, 1H), 2.95-2.86 (m, 1H), 2.64-2.54 (m, 2H), 2.11-1.94 (m, 5H), 1.83-1.73 (m, 2H), 1.71-1.61 (m, 2H).

### Example 43

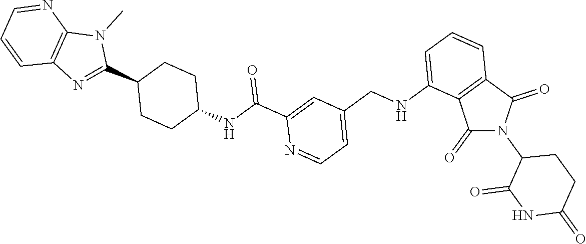 [SMILES: Cn1c2ncccc2nc1[C@H]1CC[C@H](NC(=O)c2cc(CNc3cccc4c3C(=O)N(C3CCC(=O)NC3=O)C4=O)ccn2)CC1]

### Preparation Steps

### Step 1

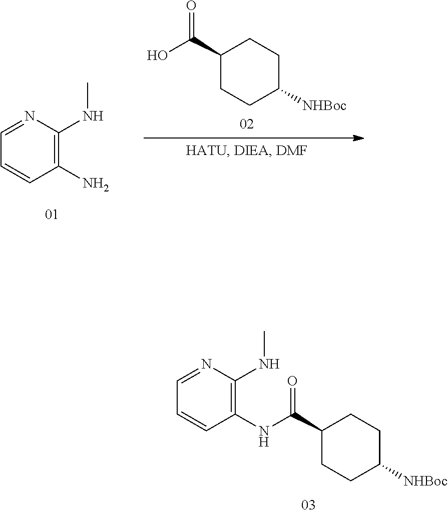

01 (200 mg, 1.62 mmol), 02 (355 mg, 1.46 mmol) and N,N-diisopropylethylamine (628 mg, 4.86 mmol) were dissolved in anhydrous N,N-dimethylformamide (3 mL), and (7-azabenzotriazole)-tetramethyluronium hexafluorophosphate (924 mg, 2.43 mmol) was added in batches with stirring at room temperature. The reaction mixture was stirred at room temperature for 16 h. The mixture was passed through a reversed-phase column (C18, 10-100% acetonitrile/water as mobile phase, 0.03% trifluoroacetic acid) to separate the target product as a red solid (290 mg, 51.2% yield). LC-MS: 349.4 [M+H]⁺.

### Step 2

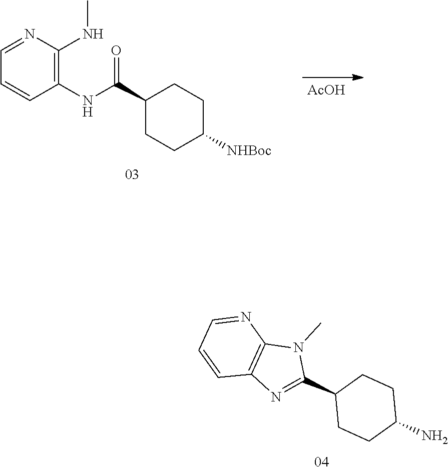 [SMILES: "AcOH" ; CNc1ncccc1NC(=O)[C@H]1CC[C@H](NC(=O)OC(C)(C)C)CC1 ; Cn1c2ncccc2nc1[C@H]1CC[C@H](N)CC1]

03 (290 mg, 0.832 mmol) and acetic acid (2 mL) were added to a sealed tube, and the reaction mixture was stirred at 130° C. for 16 h under closed conditions and concentrated under reduced pressure to remove the solvent to give the target product as a gray solid (117 mg, 61.0% yield). LC-MS: 231.4 [M+H]⁺.

### Step 3

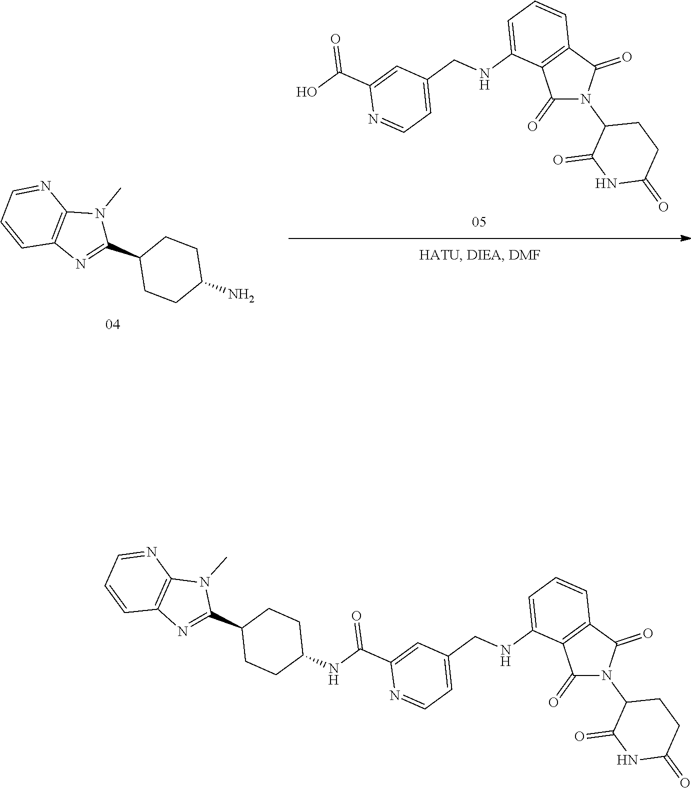 [SMILES: Cn1c2ncccc2nc1[C@H]1CC[C@H](N)CC1 ; O=C1CCC(N2C(=O)c3cccc(NCc4ccnc(C(=O)O)c4)c3C2=O)C(=O)N1 ; Cn1c2ncccc2nc1[C@H]1CC[C@H](NC(=O)c2cc(CNc3cccc4c3C(=O)N(C3CCC(=O)NC3=O)C4=O)ccn2)CC1]

04 (30.0 mg, 0.112 mmol), 05 (45.7 mg, 0.112 mmol) and N,N-diisopropylethylamine (43.4 mg, 0.336 mmol) were dissolved in anhydrous N,N-dimethylformamide (1 mL), and (7-azabenzotriazole)-tetramethyluronium hexafluorophosphate (63.9 mg, 0.168 mmol) was added in batches with stirring at room temperature. The reaction mixture was stirred at room temperature for 16 h. The mixture was purified by preparative reversed-phase liquid chromatography (C18, 5-100% acetonitrile/water as mobile phase, 0.1% formic acid) to separate the target product as a yellowish-green solid (21.06 mg, 30.2% yield). LC-MS: 621.2 [M+H]⁺,1H NMR (400 MHz, DMSO) δ 11.12 (s, 1H), 8.65-8.56 (m, 2H), 8.35 (d, J=4.9 Hz, 1H), 8.07-7.99 (m, 2H), 7.61-7.55 (m, 1H), 7.53-7.45 (m, 2H), 7.33-7.26 (m, 1H), 7.05 (d, J=7.0 Hz, 1H), 6.87 (d, J=8.6 Hz, 1H), 5.10 (dd, J=12.8, 5.4 Hz, 1H), 4.71 (d, J=6.2 Hz, 2H), 3.92-3.80 (m, 4H), 3.07 (t, J=11.9 Hz, 1H), 2.95-2.84 (m, 1H), 2.63-2.53 (m, 2H), 2.09-1.89 (m, 5H), 1.83-1.61 (m, 4H).

### Example 44

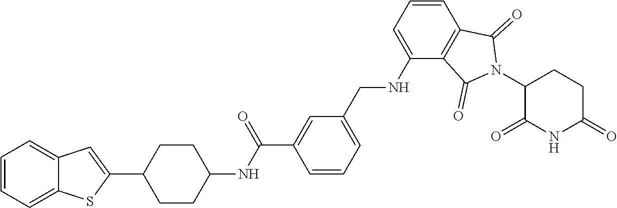 [SMILES: O=C1CCC(N2C(=O)c3cccc(NCc4cccc(C(=O)NC5CCC(c6cc7ccccc7s6)CC5)c4)c3C2=O)C(=O)N1]

### Preparation Steps

### Step 1

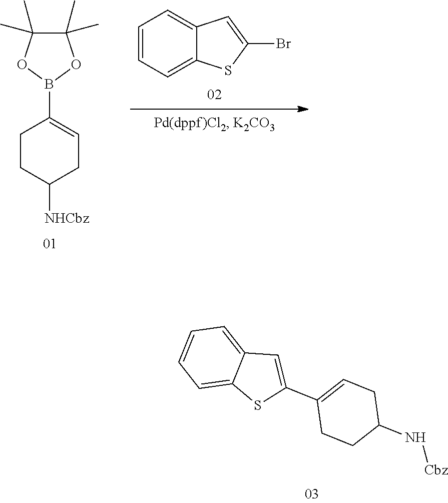

In a 25 mL single-neck flask, 01 (100 mg, 0.280 mmol) was dissolved in 1,4-dioxane (5 mL) and water (1 mL), and 02 (72 mg, 0.336 mmol), potassium carbonate (116 mg, 0.840 mmol) and Pd(dppf)Cl₂ (41 mg, 0.0560 mmol) were added. The mixture was reacted in an argon atmosphere at 80° C. for 16 h and purified by column chromatography (petroleum ether:ethyl acetate=5:1) to give the target product as a pale yellow solid (89 mg, 87.5% yield). LC-MS: 364.1 [M+H]⁺.

### Step 2

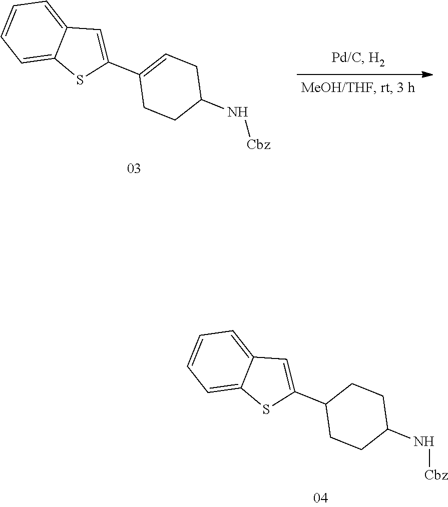 [SMILES: O=C(NC1CC=C(c2cc3ccccc3s2)CC1)OCc1ccccc1 ; O=C(NC1CCC(c2cc3ccccc3s2)CC1)OCc1ccccc1]

In a 50 mL single-neck flask, 03 (89 mg, 0.245 mmol) was dissolved in a mixed solution of methanol (5 mL) and tetrahydrofuran (1 mL), and palladium on carbon (15 mg) was added. The reaction mixture was stirred in a hydrogen atmosphere at room temperature for 3 h and filtered, and the filtrate was concentrated to dryness by rotary evaporation to give the target product as a yellow solid (82 mg, 91.8% yield). LC-MS: 366.1 [M+H]⁺.

### Step 3

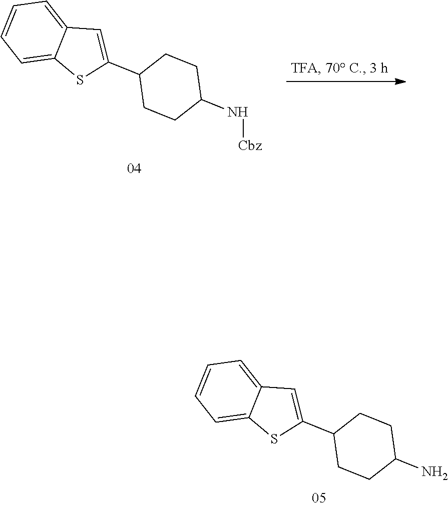 [SMILES: O=C(NC1CCC(c2cc3ccccc3s2)CC1)OCc1ccccc1 ; NC1CCC(c2cc3ccccc3s2)CC1]

In a 50 mL single-neck flask, 04 (82 mg, 0.224 mmol) was dissolved in trifluoroacetic acid (2 mL), and the reaction mixture was heated to 70° C., stirred for 2 h, and concentrated to dryness by rotary evaporation. The residue was purified by reversed-phase chromatography (acetonitrile:water=65%) to give the target product as a pale yellow solid (24 mg, 46.2% yield). LC-MS: 232.1 [M+H]⁺.

### Step 4

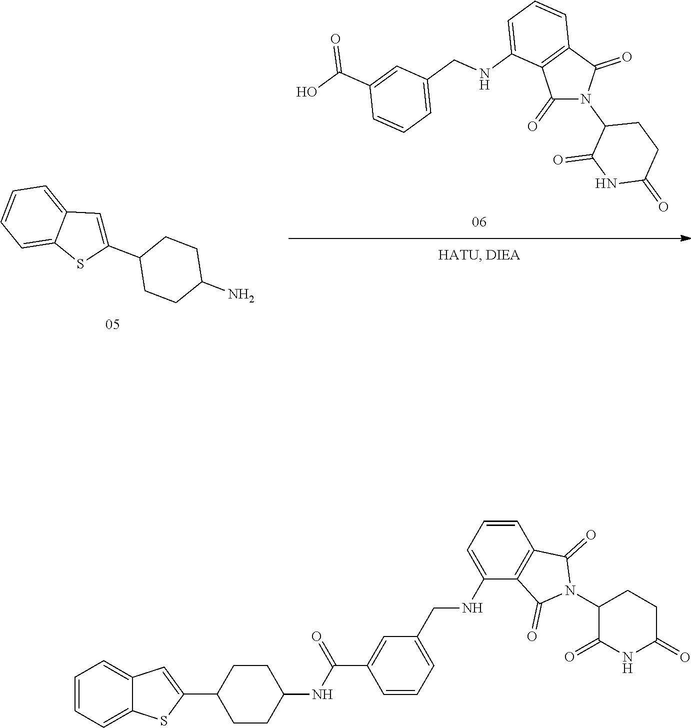 [SMILES: NC1CCC(c2cc3ccccc3s2)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6cc7ccccc7s6)CC5)c4)c3C2=O)C(=O)N1]

In a 25 mL single-neck flask, 06 (42 mg, 0.104 mmol) was dissolved in N,N-dimethylformamide (1 mL), and HATU (59 mg, 0.156 mmol), N,N-diisopropylethylamine (27 mg, 0.207 mmol) and 05 (24 mg, 0.104 mmol) were added. The mixture was reacted at room temperature for 2 h and purified by preparative high performance liquid chromatography to give the target product as a yellow solid (10.34 mg, 16.1% yield). LC-MS: 621.2 [M+H]⁺.

¹H NMR (400 MHz, DMSO-d₆) δ 11.09 (s, 1H), 8.22 (dd, J=59.7, 7.6 Hz, 1H), 7.87 (t, J=12.2 Hz, 2H), 7.72 (dd, J=15.1, 6.9 Hz, 2H), 7.55-7.46 (m, 2H), 7.41 (dd, J=15.9, 7.9 Hz, 1H), 7.36-7.31 (m, 1H), 7.30-7.25 (m, 2H), 7.20 (s, 1H), 7.02 (t, J=8.1 Hz, 1H), 6.95 (t, J=8.9 Hz, 1H), 5.20-4.96 (m, 1H), 4.72-4.50 (m, 2H), 3.93 (d, J=67.5 Hz, 1H), 3.40 (s, 1H), 3.14 (s, 1H), 2.97-2.81 (m, 2H), 2.64-2.57 (m, 1H), 2.14-2.04 (m, 2H), 2.01-1.87 (m, 2H), 1.84-1.68 (m, 2H), 1.65-1.49 (m, 2H).

### Example 45

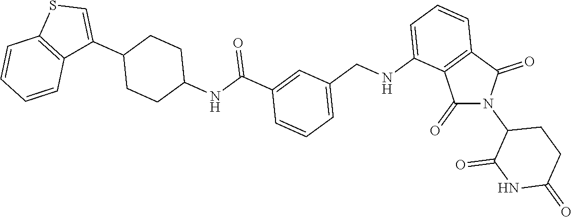 [SMILES: O=C1CCC(N2C(=O)c3cccc(NCc4cccc(C(=O)NC5CCC(c6csc7ccccc67)CC5)c4)c3C2=O)C(=O)N1]

### (1) Preparation Steps

### Step 1

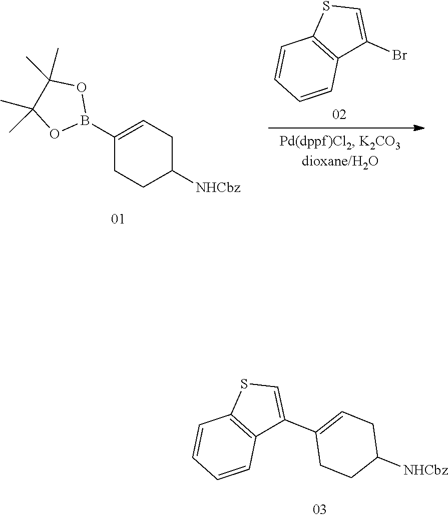 [SMILES: CC1(C)OB(C2=CCC(NC(=O)OCc3ccccc3)CC2)OC1(C)C ; Brc1csc2ccccc12 ; O=C(NC1CC=C(c2csc3ccccc23)CC1)OCc1ccccc1]

Starting materials 01 (200 mg, 0.56 mmol), 02 (143 mg, 0.67 mmol) and dichloro[1,1′-bis(diphenylphosphino)ferrocene]palladium (41 mg, 0.06 mmol) were dissolved in 1,4-dioxane (4 mL), and a solution of potassium carbonate (194 mg, 1.4 mmol) in water (1 mL) was added. The reaction mixture was stirred in an argon atmosphere at 90° C. for 3 h. Water (50 mL) was added, followed by extraction with ethyl acetate (50 mL×3). The organic phase was washed with saturated brine (50 mL), dried over anhydrous sodium sulfate, and filtered, and the filtrate was concentrated to dryness by rotary evaporation. The residue was passed through a silica gel column (petroleum ether:ethyl acetate=100:0 to 80:20) and a reversed-phase column (C18, water:acetonitrile=100:0 to 35:65, 0.1% formic acid) to separate the target product as a white solid (110 mg, 54.1% yield). LC-MS: 364.1 [M+H]⁺.

### Step 2

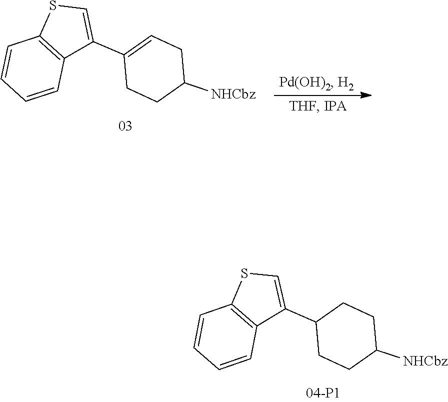 [SMILES: O=C(NC1CC=C(c2csc3ccccc23)CC1)OCc1ccccc1 ; O=C(NC1CCC(c2csc3ccccc23)CC1)OCc1ccccc1]

03 (110 mg, 0.30 mmol) was dissolved in tetrahydrofuran (1 mL) and isopropanol (1 mL) at room temperature, and palladium hydroxide on carbon (50 mg) was added. The reaction mixture was purged with hydrogen twice, stirred in a hydrogen atmosphere at room temperature for 16 h, and filtered. The filtrate was concentrated to dryness by rotary evaporation, and the residue was purified by TLC (petroleum ether:ethyl acetate=4:1) to separate the target product as a white solid (upper spot, 55 mg, 50.2% yield). LC-MS: 366.1 [M+H]⁺.

### Step 3

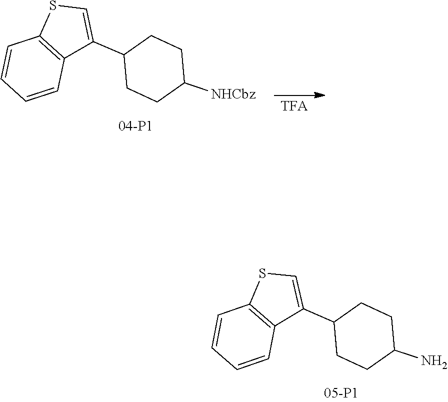 [SMILES: O=C(NC1CCC(c2csc3ccccc23)CC1)OCc1ccccc1 ; NC1CCC(c2csc3ccccc23)CC1]

04 (55 mg, 0.15 mmol) was dissolved in trifluoroacetic acid (1 mL) at room temperature, and the reaction mixture was stirred in an argon atmosphere at 70° C. for 0.5 h. The solvent was removed by rotary evaporation, and the residue was passed through a reversed-phase column (C18, water:acetonitrile=100:0 to 50:50, 0.1% formic acid) to separate the target product as a white solid (15 mg, 43.3% yield). LC-MS: 232.3 [M+H]⁺.

### Step 4

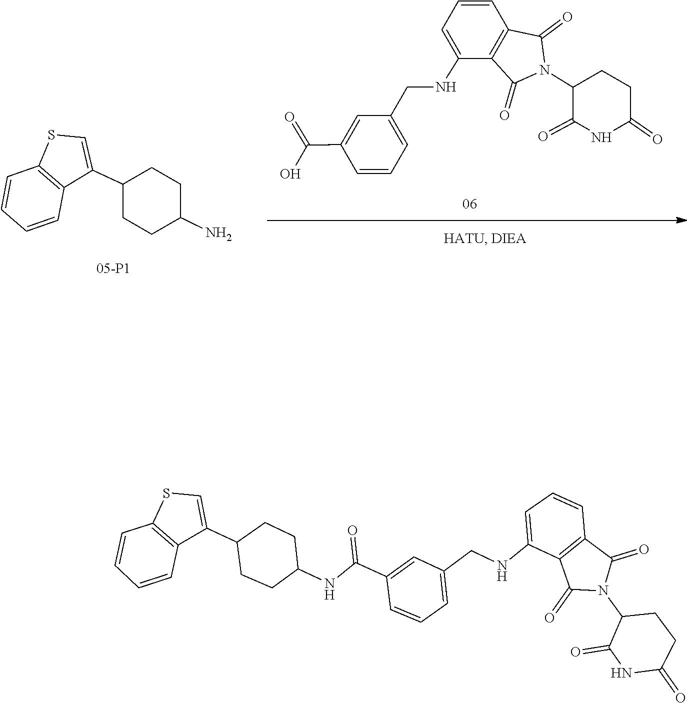 [SMILES: NC1CCC(c2csc3ccccc23)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6csc7ccccc67)CC5)c4)c3C2=O)C(=O)N1]

05-P1 (15 mg, 0.065 mmol), 06 (27 mg, 0.065 mmol) and N,N-diisopropylethylamine (26 mg, 0.195 mmol) were dissolved in N,N-dimethylformamide (0.5 mL) at room temperature, and (7-azabenzotriazole)-tetramethyluronium hexafluorophosphate (30 mg, 0.078 mmol) was added with stirring at room temperature. The reaction mixture was stirred at room temperature for 2 h. The mixture was purified by preparative chromatography (C18, 5-100% acetonitrile/water as mobile phase, 0.1% formic acid) to separate the target product as a yellow solid (11.06 mg, 27.4% yield). LC-MS: 621.3 [M+H]⁺. ¹H NMR (400 MHz, DMSO-d₆) δ 11.09 (s, 1H), 8.15 (d, J=6.8 Hz, 1H), 7.97 (d, J=7.6 Hz, 1H), 7.90-7.81 (m, 2H), 7.74 (d, J=7.6 Hz, 1H), 7.54-7.48 (m, 2H), 7.45-7.34 (m, 4H), 7.29 (t, J=5.9 Hz, 1H), 7.02 (d, J=7.0 Hz, 1H), 6.96 (d, J=8.6 Hz, 1H), 5.06 (dd, J=12.8, 5.3 Hz, 1H), 4.62 (d, J=5.9 Hz, 2H), 4.19-4.11 (m, 1H), 3.14-3.03 (m, 1H), 2.93-2.82 (m, 1H), 2.63-2.53 (m, 2H), 2.07-2.01 (m, 1H), 1.99-1.88 (m, 4H), 1.87-1.74 (m, 4H).

### Example 46

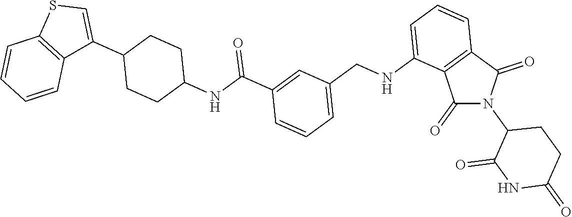 [SMILES: O=C1CCC(N2C(=O)c3cccc(NCc4cccc(C(=O)NC5CCC(c6csc7ccccc67)CC5)c4)c3C2=O)C(=O)N1]

### Preparation Steps

### Step 1

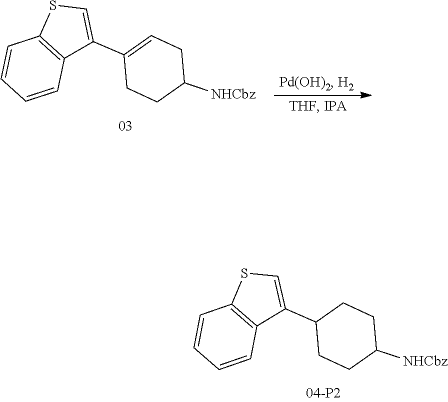 [SMILES: O=C(NC1CC=C(c2csc3ccccc23)CC1)OCc1ccccc1 ; O=C(NC1CCC(c2csc3ccccc23)CC1)OCc1ccccc1]

03 (110 mg, 0.30 mmol) was dissolved in tetrahydrofuran (1 mL) and isopropanol (1 mL) at room temperature, and palladium hydroxide on carbon (50 mg) was added. The reaction mixture was purged with hydrogen twice, stirred in a hydrogen atmosphere at room temperature for 16 h, and filtered. The filtrate was concentrated to dryness by rotary evaporation, and the residue was purified by TLC (petroleum ether:ethyl acetate=4:1) to separate the target product as a white solid (lower spot, 25 mg, 22.7% yield). LC-MS: 366.1 [M+H]⁺.

### Step 2

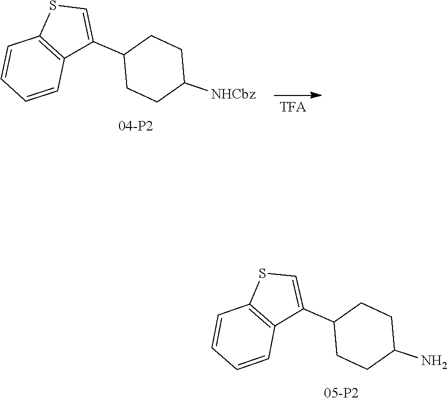 [SMILES: O=C(NC1CCC(c2csc3ccccc23)CC1)OCc1ccccc1 ; NC1CCC(c2csc3ccccc23)CC1]

04 (25 mg, 0.0684 mmol) was dissolved in trifluoroacetic acid (1 mL) at room temperature, and the reaction mixture was stirred in an argon atmosphere at 70° C. for 0.5 h. The solvent was removed by rotary evaporation, and the residue was passed through a reversed-phase column (C18, water:acetonitrile=100:0 to 50:50, 0.1% formic acid) to separate the target product as a white solid (10 mg, 63.2% yield). LC-MS: 232.3 [M+H]⁺.

### Step 3

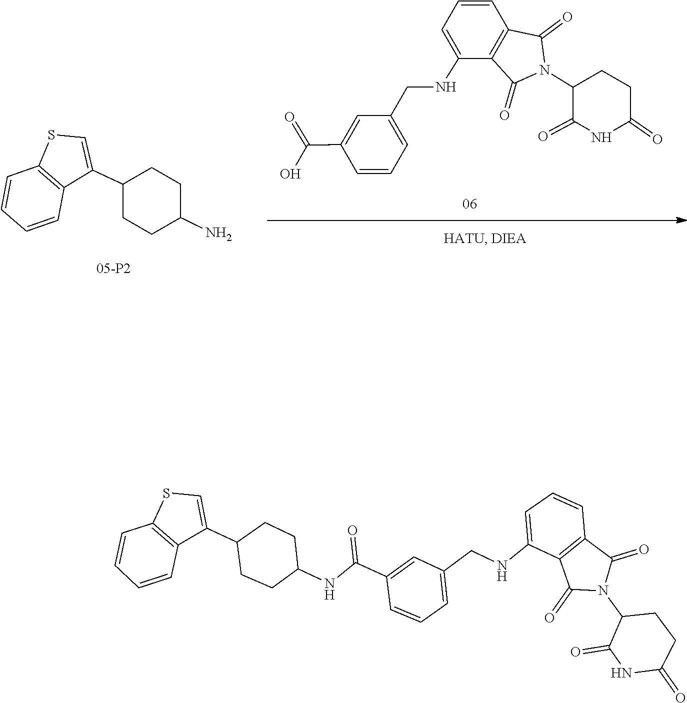 [SMILES: NC1CCC(c2csc3ccccc23)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6csc7ccccc67)CC5)c4)c3C2=O)C(=O)N1]

05-P2 (10 mg, 0.043 mmol), 06 (17.6 mg, 0.043 mmol) and N,N-diisopropylethylamine (16.6 mg, 0.129 mmol) were dissolved in N,N-dimethylformamide (0.5 mL) at room temperature, and (7-azabenzotriazole)-tetramethyluronium hexafluorophosphate (19.6 mg, 0.0516 mmol) was added with stirring at room temperature. The reaction mixture was stirred at room temperature for 2 h. The mixture was purified by preparative chromatography (C18, 5-100% acetonitrile/water as mobile phase, 0.1% formic acid) to separate the target product as a yellow solid (product 2: 7.27 mg, 27.2% yield). LC-MS: 621.3 [M+H]⁺. ¹H NMR (400 MHz, DMSO-d₆) δ 11.10 (s, 1H), 8.32 (d, J=8.1 Hz, 1H), 7.97 (d, J=7.4 Hz, 1H), 7.91-7.86 (m, 2H), 7.75 (d, J=6.9 Hz, 1H), 7.54-7.49 (m, 2H), 7.45-7.34 (m, 4H), 7.32-7.27 (m, 1H), 7.04 (d, J=7.1 Hz, 1H), 6.96 (d, J=8.3 Hz, 1H), 5.08 (dd, J=12.6, 5.2 Hz, 1H), 4.62 (d, J=5.8 Hz, 2H), 3.95-3.84 (m, 1H), 2.98-2.91 (m, 1H), 2.91-2.84 (m, 1H), 2.65-2.56 (m, 2H), 2.08-2.04 (m, 2H), 2.02-1.96 (m, 3H), 1.67-1.59 (m, 4H).

### Example 47

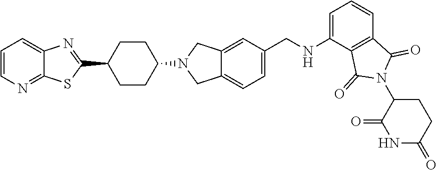 [SMILES: O=C1CCC(N2C(=O)c3cccc(NCc4ccc5c(c4)CN([C@H]4CC[C@H](c6nc7cccnc7s6)CC4)C5)c3C2=O)C(=O)N1]

### Preparation Steps

### Step 1

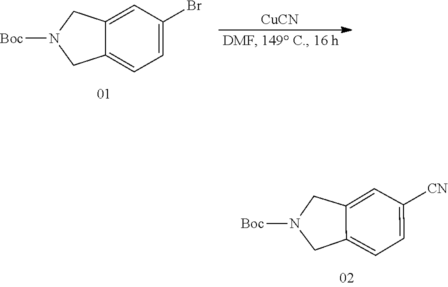

01 (854 mg, 2.864 mmol) and cuprous cyanide (1282 mg, 14.32 mmol) were dissolved in N,N-dimethylformamide (25 mL) at room temperature, and the reaction mixture was stirred at 149° C. for 16 h. Water (50 mL) was added, followed by extraction with ethyl acetate (50 mL×3). The organic phase was washed with saturated brine, dried over anhydrous sodium sulfate, and filtered, and the filtrate was concentrated to dryness by rotary evaporation. The residue was passed through a silica gel column (petroleum ether:ethyl acetate=2:1) to separate the target product as a white solid (249 mg, 35.5% yield). LC-MS: 189.1 [M+H−56]⁺.

### Step 2

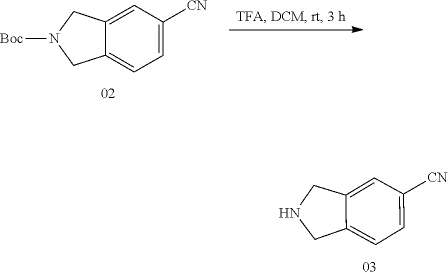 [SMILES: CC(C)(C)OC(=O)N1Cc2ccc(C#N)cc2C1 ; N#Cc1ccc2c(c1)CNC2]

02 (100 mg, 0.41 mmol) was dissolved in dichloromethane (4 mL) and trifluoroacetic acid (0.8 mL), and the reaction mixture was stirred at room temperature for 2 h and concentrated to dryness by rotary evaporation to give the target product as a white solid (100 mg, crude). LC-MS: 145.1 [M+H]⁺.

### Step 3

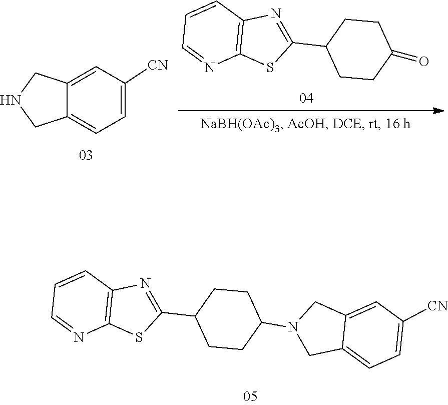

03 (100 mg, 0.69 mmol) and 04 (161 mg, 0.69 mmol) were dissolved in dichloroethane (5 mL) at room temperature, and sodium triacetoxyborohydride (441 mg, 2.08 mmol) was added with stirring at room temperature. The reaction mixture was stirred at room temperature for 12 h and concentrated to dryness by rotary evaporation. The residue was purified through a silica gel column (petroleum ether:ethyl acetate=2:1) to give the target product as a white solid (100 mg, 94.5% yield over two steps). LC-MS: 361.3 [M+H]⁺.

### Step 4

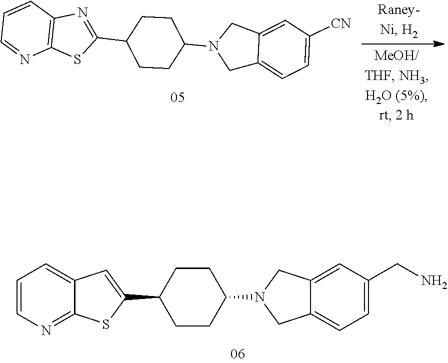 [SMILES: N#Cc1ccc2c(c1)CN(C1CCC(c3nc4cccnc4s3)CC1)C2 ; NCc1ccc2c(c1)CN([C@H]1CC[C@H](c3cc4cccnc4s3)CC1)C2]

05 (100 mg, 0.25 mmol) was dissolved in methanol (2.5 mL) at room temperature, and tetrahydrofuran (2.5 mL), ammonia solution (0.5 mL) and Raney-Ni were added. The reaction mixture was stirred in a hydrogen atmosphere at room temperature for 2 h and filtered, and the filtrate was concentrated to dryness by rotary evaporation to give the target product as a white solid (100 mg, 99% yield). LC-MS: 365.4 [M+H]⁺.

### Step 5

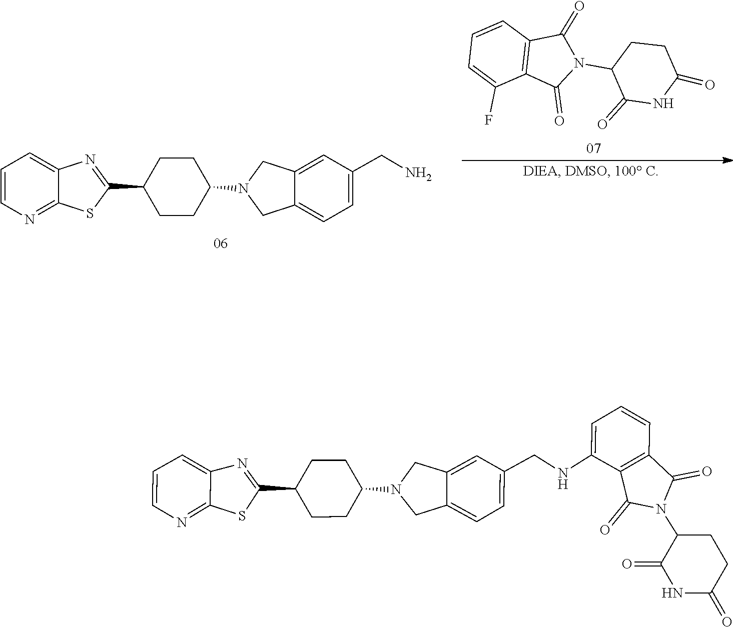 [SMILES: O=C1CCC(N2C(=O)c3cccc(F)c3C2=O)C(=O)N1 ; NCc1ccc2c(c1)CN([C@H]1CC[C@H](c3nc4cccnc4s3)CC1)C2 ; O=C1CCC(N2C(=O)c3cccc(NCc4ccc5c(c4)CN([C@H]4CC[C@H](c6nc7cccnc7s6)CC4)C5)c3C2=O)C(=O)N1]

06 (100 mg, 0.27 mmol) and 07 (76 mg, 0.27 mmol) were dissolved in dimethyl sulfoxide (1.5 mL) at room temperature, and N,N-diisopropylethylamine (106 mg, 0.82 mmol) was added at room temperature. The reaction mixture was stirred at 100° C. for 12 h and purified by preparative chromatography to separate the target product as a green solid (3.15 mg, 1.9% yield). LC-MS: 621.3 [M+H]⁺. ¹H NMR (400 MHz, DMSO-d₆) δ 11.09 (s, 1H), 8.59-8.53 (m, 1H), 8.35-8.28 (m, 1H), 7.58-7.48 (m, 2H), 7.21 (dd, J=18.8, 12.5 Hz, 4H), 7.02 (t, J=5.4 Hz, 1H), 6.96 (d, J=8.6 Hz, 1H), 5.06 (dd, J=12.9, 5.4 Hz, 1H), 4.53 (d, J=5.9 Hz, 2H), 3.87 (d, J=19.5 Hz, 4H), 2.87 (d, J=13.7 Hz, 1H), 2.59 (d, J=16.0 Hz, 4H), 2.18-1.99 (m, 4H), 1.91 (s, 3H), 1.71 (t, J=12.4 Hz, 2H).

### Example 48

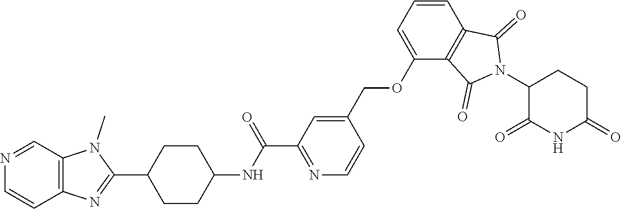 [SMILES: Cn1c(C2CCC(NC(=O)c3cc(COc4cccc5c4C(=O)N(C4CCC(=O)NC4=O)C5=O)ccn3)CC2)nc2ccncc21]

### Preparation Steps

### Step 1

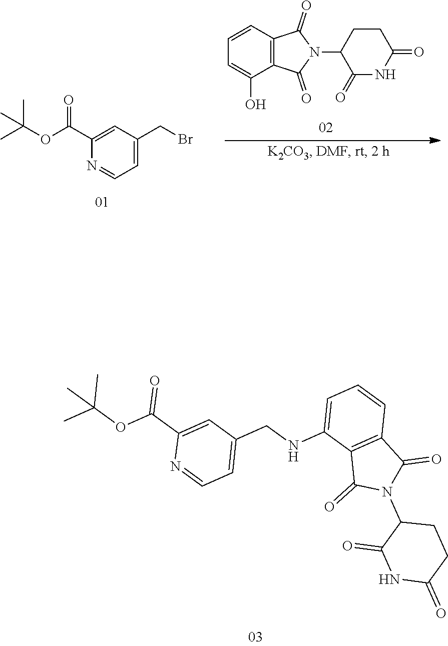 [SMILES: CC(C)(C)OC(=O)c1cc(CBr)ccn1 ; O=C1CCC(N2C(=O)c3cccc(O)c3C2=O)C(=O)N1 ; CC(C)(C)OC(=O)c1cc(CNc2cccc3c2C(=O)N(C2CCC(=O)NC2=O)C3=O)ccn1]

To a 50 mL single-neck flask were added 01 (78 mg, 0.287 mmol), 02 (78.7 mg, 0.287 mmol), potassium carbonate (39.7 mg, 0.287 mmol) and N,N-dimethylformamide (2 mL), and the mixture was reacted at room temperature for 2 h, quenched with water (10 mL), and extracted with ethyl acetate (10 mL×2). The organic phase was washed with saturated brine (10 mL×3), dried over anhydrous sodium sulfate, and concentrated to dryness by rotary evaporation, and the residue was purified by column chromatography (methanol:dichloromethane=10:1) to give a yellow solid (60 mg, 44.9% yield). LC-MS: 466.4 [M+H]⁺.

### Step 2

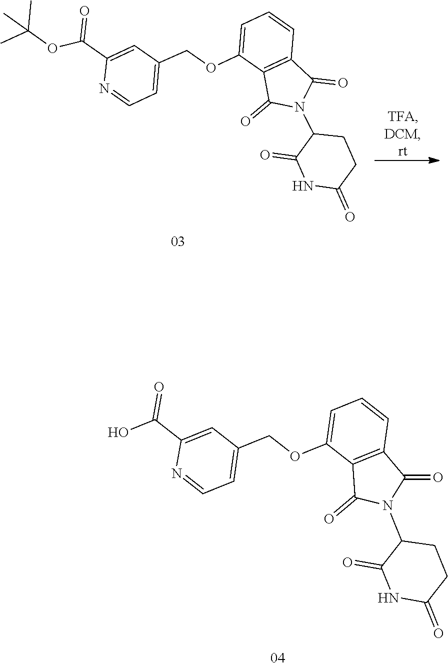 [SMILES: CC(C)(C)OC(=O)c1cc(COc2cccc3c2C(=O)N(C2CCC(=O)NC2=O)C3=O)ccn1 ; O=C1CCC(N2C(=O)c3cccc(OCc4ccnc(C(=O)O)c4)c3C2=O)C(=O)N1]

To a 50 mL single-neck flask were added 03 (30 mg, 0.064 mmol), dichloromethane (3 mL) and trifluoroacetic acid (1 mL), and the mixture was reacted at room temperature for 12 h and concentrated by rotary evaporation to remove the solvent to give the target product (20 mg, 76.3% yield). LC-MS: 410.3 [M+H]⁺.

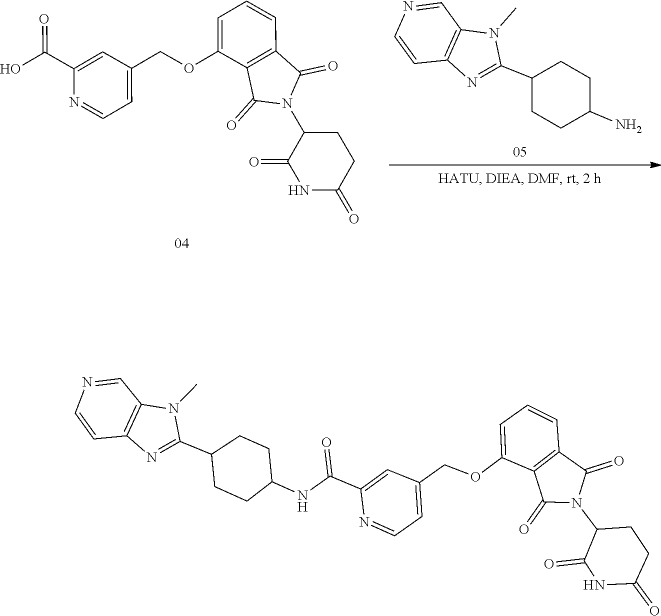 [SMILES: O=C1CCC(N2C(=O)c3cccc(OCc4ccnc(C(=O)O)c4)c3C2=O)C(=O)N1 ; Cn1c(C2CCC(N)CC2)nc2ccncc21 ; Cn1c(C2CCC(NC(=O)c3cc(COc4cccc5c4C(=O)N(C4CCC(=O)NC4=O)C5=O)ccn3)CC2)nc2ccncc21]

To a 50 mL single-neck flask were added 04 (20 mg, 0.0489 mmol), 05 (13.5 mg, 0.0587 mmol), HATU (27.9 mg, 0.0733 mmol), DIEA (19.0 mg, 0.147 mmol) and DMF (1 mL), and the mixture was reacted at room temperature for 2 h. The reaction mixture was purified by preparative high performance liquid chromatography to give a white solid (10.93 mg, 35.9% yield). LC-MS: 622.5 [M+H]⁺. ¹H NMR (400 MHz, DMSO-d6) δ 11.12 (s, 1H), 9.42 (s, 1H), 8.78-8.42 (m, 3H), 8.27-8.08 (m, 2H), 7.86 (t, J=7.9 Hz, 1H), 7.76 (t, J=5.9 Hz, 1H), 7.55 (dd, J=19.2, 7.9 Hz, 2H), 5.56 (s, 2H), 5.13 (dd, J=12.9, 5.3 Hz, 1H), 4.14-3.99 (m, 3H), 3.99-3.87 (m, 1H), 3.23-3.10 (m, 1H), 2.96-2.84 (m, 1H), 2.68-2.55 (m, 2H), 2.16-1.94 (m, 5H), 1.93-1.60 (m, 4H).

### Example 49

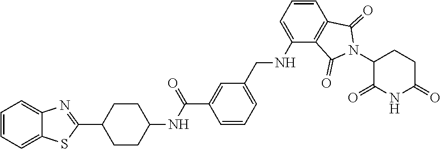 [SMILES: O=C1CCC(N2C(=O)c3cccc(NCc4cccc(C(=O)NC5CCC(c6nc7ccccc7s6)CC5)c4)c3C2=O)C(=O)N1]

### Preparation Steps

### Step 1

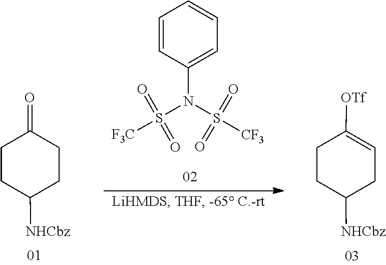 [SMILES: O=C1CCC(NC(=O)OCc2ccccc2)CC1 ; O=S(=O)(N(c1ccccc1)S(=O)(=O)C(F)(F)F)C(F)(F)F ; O=C(NC1CC=C(OS(=O)(=O)C(F)(F)F)CC1)OCc1ccccc1]

4-(Benzyloxyamido)cyclohexanone (12 g, 48.5 mmol) was dissolved in tetrahydrofuran (150 mL), and a 1 M solution of lithium bis(trimethylsilyl)amide in tetrahydrofuran (97.0 mL, 97.0 mmol) was added at −65° C. The reaction mixture was stirred at −65° C. for 1 h, and a solution of N-phenyl bis(trifluoromethanesulfonyl)imide (17.3 g, 48.5 mmol) in tetrahydrofuran (40 mL) was added dropwise. The reaction mixture was stirred at room temperature for 16 h. The mixture was quenched by slowly pouring into water (150 mL) and extracted with ethyl acetate (150 mL×2). The organic phase was washed with saturated brine (200 mL×2), dried over anhydrous sodium sulfate, and filtered, and the filtrate was concentrated to dryness by rotary evaporation. The residue was purified through a flash silica gel column (petroleum ether:ethyl acetate=10:1) to give a crude product, which was further slurried with petroleum ether:ethyl acetate (102 mL, 50:1) to give the target product as a white solid (5.5 g, 29.9% yield). LC-MS: 380.2 [M+H]⁺.

### Step 2

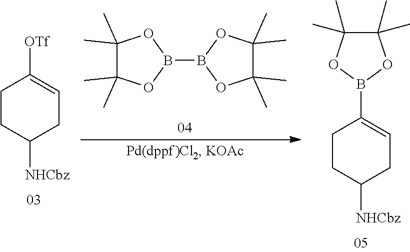

03 (9.0 g, 23.7 mmol), bis(pinacolato)diboron (7.23 g, 28.5 mmol), potassium acetate (6.99 g, 71.2 mmol) and [1,1′-bis(diphenylphosphino)ferrocene]palladium (II) dichloride (868 mg, 1.19 mmol) were mixed in anhydrous dioxane (150 mL), and the reaction mixture was purged with argon, stirred in an argon atmosphere at 90° C. for 3 h, cooled to room temperature, concentrated under reduced pressure to remove the solvent, diluted with ethyl acetate (300 mL), and filtered. The filtrate was washed with water (150 mL) and saturated brine (150 mL), dried over anhydrous sodium sulfate, and filtered, and the filtrate was concentrated to dryness by rotary evaporation. The residue was purified through a flash silica gel column (petroleum ether:ethyl acetate=10:1) to give the target product as an off-white solid (6.3 g, 74.3% yield). LC-MS: 358.2 [M+H]⁺.

### Step 3

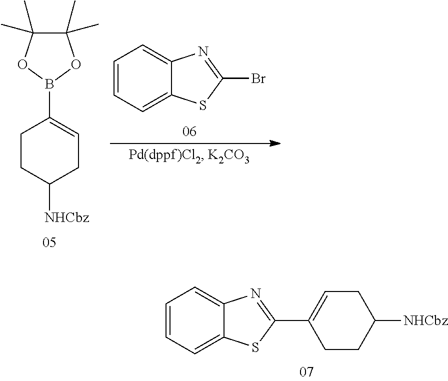

05 (100 mg, 0.280 mmol), 2-bromobenzothiazole (59.9 mg, 0.280 mmol), potassium carbonate (116 mg, 0.840 mmol) and [1,1′-bis(diphenylphosphino)ferrocene]palladium (II) dichloride (20.4 mg, 0.0280 mmol) were mixed in dioxane (5 mL) and water (1 mL), and the reaction mixture was purged with argon, stirred in an argon atmosphere at 100° C. for 7 h, cooled to room temperature, and then concentrated under reduced pressure to remove the solvent. Water (10 mL) was added, followed by extraction with ethyl acetate (5 mL×2). The organic phase was washed with saturated brine (10 mL), dried over anhydrous sodium sulfate, and filtered, and the filtrate was concentrated to dryness by rotary evaporation. The residue was purified through a flash silica gel column (petroleum ether:ethyl acetate=5:1) to give the target product as a yellow solid (60 mg, 58.8% yield). LC-MS: 365.2 [M+H]⁺.

### Step 4

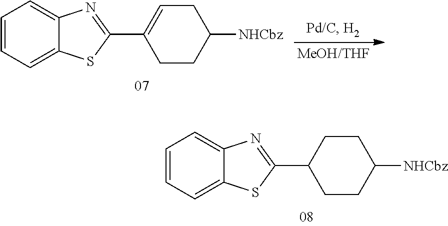 [SMILES: O=C(NC1CC=C(c2nc3ccccc3s2)CC1)OCc1ccccc1 ; O=C(NC1CCC(c2nc3ccccc3s2)CC1)OCc1ccccc1]

07 (60 mg, 0.165 mmol) was dissolved in tetrahydrofuran (2 mL) and methanol (0.5 mL), and wet palladium on carbon (20 mg, containing 10% palladium and 50% water) was added. The reaction mixture was purged with hydrogen, stirred in a hydrogen atmosphere at room temperature for 14 h, and filtered, and the filtrate was concentrated to dryness by rotary evaporation to give the target product as a yellow solid (55 mg, 91.2% yield). LC-MS: 367.3 [M+H]⁺.

### Step 5

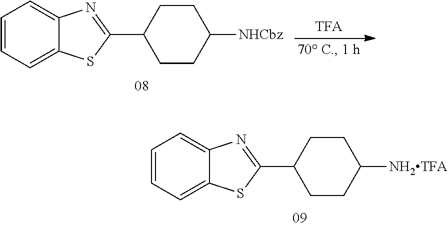

08 (55 mg, 0.150 mmol) was dissolved in trifluoroacetic acid (1 mL), and the reaction mixture was stirred at 70° C. for 1 h and concentrated under reduced pressure to remove the solvent to give the target product as a brownish-yellow oil (52.0 mg, 100% yield). LC-MS: 233.3 [M+H]⁺.

### Step 6

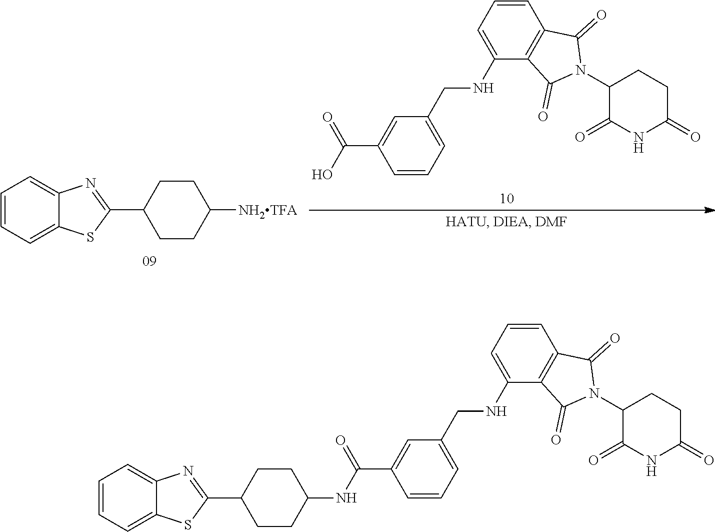

09 (52.0 mg, 0.150 mmol), 10 (61.1 mg, 0.150 mmol) and N,N-diisopropylethylamine (96.9 mg, 0.750 mmol) were dissolved in anhydrous N,N-dimethylformamide (1 mL), and (7-azabenzotriazole)-tetramethyluronium hexafluorophosphate (68.4 mg, 0.180 mmol) was added in batches with stirring at room temperature. The reaction mixture was stirred at room temperature for 2 h. The mixture was purified by preparative chromatography (C18, 5-100% acetonitrile/water as mobile phase, 0.1% hydrochloric acid) to separate the target product as a yellow solid (45 mg, 48.2% yield). LC-MS: 622.1 [M+H]⁺. ¹H NMR (400 MHz, DMSO-d₆) δ 11.13-11.08 (m, 1H), 8.35-8.14 (m, 1H), 8.10-8.05 (m, 1H), 7.99-7.93 (m, 1H), 7.89-7.83 (m, 1H), 7.76-7.67 (m, 1H), 7.54-7.45 (m, 3H), 7.44-7.34 (m, 2H), 7.33-7.23 (m, 1H), 7.06-6.99 (m, 1H), 6.98-6.91 (m, 1H), 5.13-5.03 (m, 1H), 4.66-4.54 (m, 2H), 4.04-3.86 (m, 1H), 3.39-3.08 (m, 1H), 2.96-2.83 (m, 1H), 2.66-2.52 (m, 2H), 2.35-2.19 (m, 2H), 2.09-1.95 (m, 3H), 1.80-1.67 (m, 3H), 1.62-1.51 (m, 1H).

### Example 50

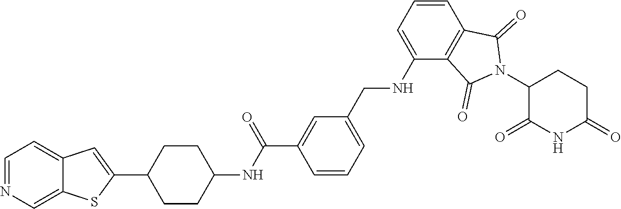 [SMILES: O=C1CCC(N2C(=O)c3cccc(NCc4cccc(C(=O)NC5CCC(c6cc7ccncc7s6)CC5)c4)c3C2=O)C(=O)N1]

### Preparation Steps

### Step 1

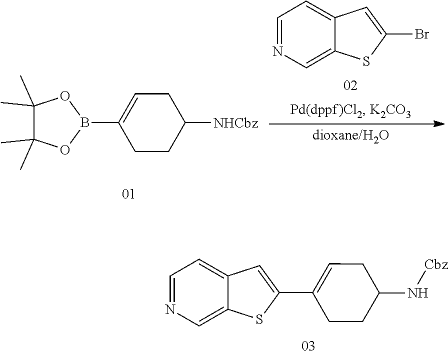 [SMILES: Brc1cc2ccncc2s1 ; CC1(C)OB(C2=CCC(NC(=O)OCc3ccccc3)CC2)OC1(C)C ; O=C(NC1CC=C(c2cc3ccncc3s2)CC1)OCc1ccccc1]

To a 100 mL single-neck flask were added 01 (100 mg, 0.279 mmol), 02 (60 mg, 0.279 mmol), potassium carbonate (116 mg, 0.839 mmol), Pd(dppf)Cl₂ (20 mg, 0.0279 mmol), dioxane (4 mL) and water (1 mL), and the reaction mixture was stirred in a nitrogen atmosphere at 100° C. for 16 h, cooled to room temperature, and concentrated to dryness by rotary evaporation to give a crude product, which was then purified by Pre-TLC (PE:EA=1:3) to give the target product as a white solid (83 mg, 81.4% yield). LC-MS: 365.1 [M+H]⁺.

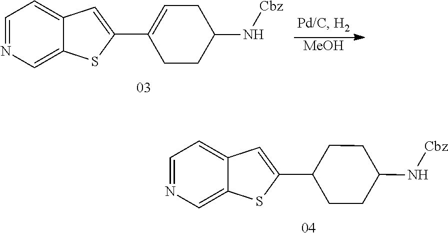 [SMILES: "MeOH" ; O=C(NC1CC=C(c2cc3ccncc3s2)CC1)OCc1ccccc1 ; O=C(NC1CCC(c2cc3ccncc3s2)CC1)OCc1ccccc1]

In a 100 mL single-neck flask, 03 (40 mg, 0.109 mmol) was dissolved in methanol (3 mL), and palladium on carbon (10 mg) was added. The reaction mixture was stirred in a hydrogen atmosphere for 16 h and filtered, and the filtrate was concentrated to dryness by rotary evaporation to give the target product as a white solid (35 mg, 87.0% yield), which was directly used in the next step.

LC-MS: 367.1 [M+H]⁺.

### Step 3

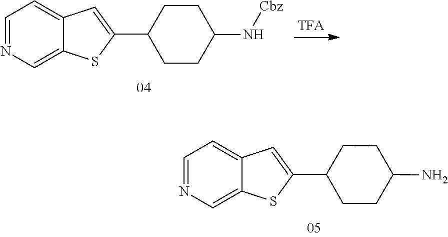 [SMILES: O=C(NC1CCC(c2cc3ccncc3s2)CC1)OCc1ccccc1 ; NC1CCC(c2cc3ccncc3s2)CC1]

04 (35 mg, 0.095 mmol) was dissolved in trifluoroacetic acid (3 mL) at room temperature, and the reaction mixture was stirred in a nitrogen atmosphere at 70° C. for 3 h and concentrated to dryness by rotary evaporation to give the target product as a yellow oil (20 mg, 90.1% yield).

LC-MS: 233.1 [M+H]⁺.

### Step 4

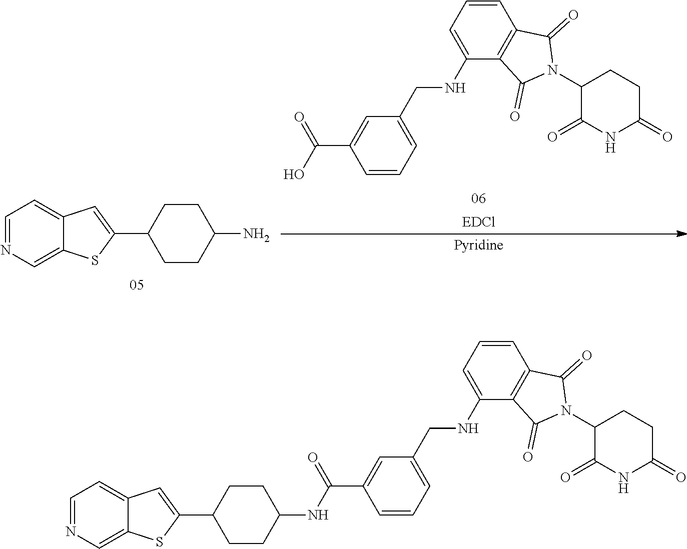 [SMILES: O=C1CCC(N2C(=O)c3cccc(NCc4cccc(C(=O)O)c4)c3C2=O)C(=O)N1 ; NC1CCC(c2cc3ccncc3s2)CC1 ; O=C1CCC(N2C(=O)c3cccc(NCc4cccc(C(=O)NC5CCC(c6cc7ccncc7s6)CC5)c4)c3C2=O)C(=O)N1]

05 (20 mg, 0.086 mmol) was dissolved in pyridine (1 mL) at room temperature, and 06 (35 mg, 0.086 mmol) and EDCI (49 mg, 0.258 mmol) were added. The reaction mixture was stirred at room temperature for 16 h and concentrated to give a crude product, which was then purified by preparative high performance liquid chromatography to give the target product as a yellow solid (6.35 mg, 11.9% yield). LC-MS: 622.1 [M+H]⁺, ¹H NMR (400 MHz, DMSO-d₆) δ 11.10 (s, 1H), 9.13 (s, 1H), 8.42 (d, J=5.4 Hz, 2H), 8.32 (d, J=7.8 Hz, 1H), 8.16 (d, J=7.2 Hz, 1H), 7.85 (d, J=8.0 Hz, 1H), 7.77-7.66 (m, 2H), 7.52-7.50 (m, 2H), 7.45-7.36 (m, 2H), 7.34-7.24 (m, 2H), 7.03 (t, J=7.4 Hz, 1H), 6.95 (t, J=8.9 Hz, 1H), 5.16-5.00 (m, 1H), 4.61 (dd, J=10.1, 6.3 Hz, 2H), 3.99-3.85 (m, 1H), 3.00 (s, 1H), 2.89-2.80 (m, 1H), 2.64-2.54 (m, 2H), 2.10-1.80 (m, 5H), 1.76 (s, 2H), 1.68-1.52 (m, 2H).

### Example 51

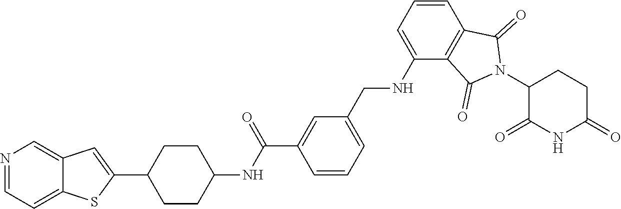 [SMILES: O=C1CCC(N2C(=O)c3cccc(NCc4cccc(C(=O)NC5CCC(c6cc7cnccc7s6)CC5)c4)c3C2=O)C(=O)N1]

### Preparation Steps

### Step 1

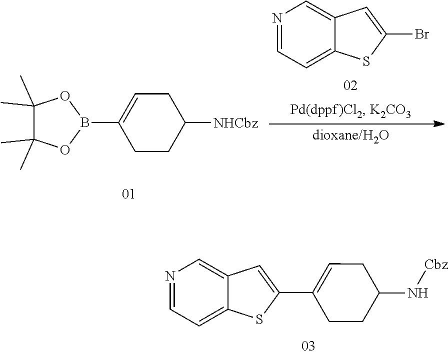 [SMILES: Brc1cc2cnccc2s1 ; CC1(C)OB(C2=CCC(NC(=O)OCc3ccccc3)CC2)OC1(C)C ; O=C(NC1CC=C(c2cc3cnccc3s2)CC1)OCc1ccccc1]

To a 100 mL single-neck flask were added 01 (166 mg, 0.467 mmol), 02 (100 mg, 0.467 mmol), potassium carbonate (193 mg, 1.401 mmol), Pd(dppf)Cl₂ (34 mg, 0.0467 mmol), dioxane (4 mL) and water (1 mL), and the reaction mixture was stirred in a nitrogen atmosphere at 100° C. for 16 h, cooled to room temperature, and concentrated to dryness by rotary evaporation to give a crude product, which was then purified by Pre-TLC (PE:EA=1:3) to give the target product as a white solid (120 mg, 70.9% yield). LC-MS: 365.0 [M+H]⁺.

### Step 2

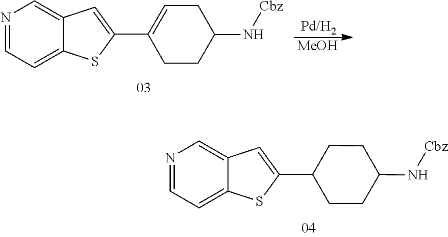

In a 100 mL single-neck flask, 03 (120 mg, 0.329 mmol) was dissolved in methanol (3 mL), and palladium on carbon (10 mg) was added. The reaction mixture was stirred in a hydrogen atmosphere for 16 h and filtered, and the filtrate was concentrated to dryness by rotary evaporation to give the target product as a white solid (103 mg, 85.4% yield), which was directly used in the next step. LC-MS: 367.0 [M+H]⁺.

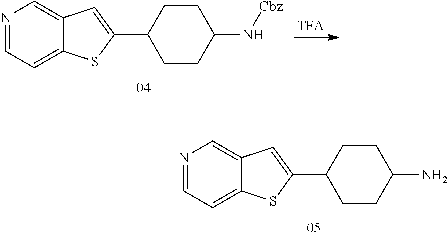 [SMILES: O=C(NC1CCC(c2cc3cnccc3s2)CC1)OCc1ccccc1 ; NC1CCC(c2cc3cnccc3s2)CC1]

04 (103 mg, 0.281 mmol) was dissolved in trifluoroacetic acid (5 mL) at room temperature, and the reaction mixture was stirred in a nitrogen atmosphere at 70° C. for 3 h and concentrated to dryness by rotary evaporation to give the target product as a yellow oil (55 mg, 84.2% yield). LC-MS: 233.1 [M+H]⁺.

### Step 4

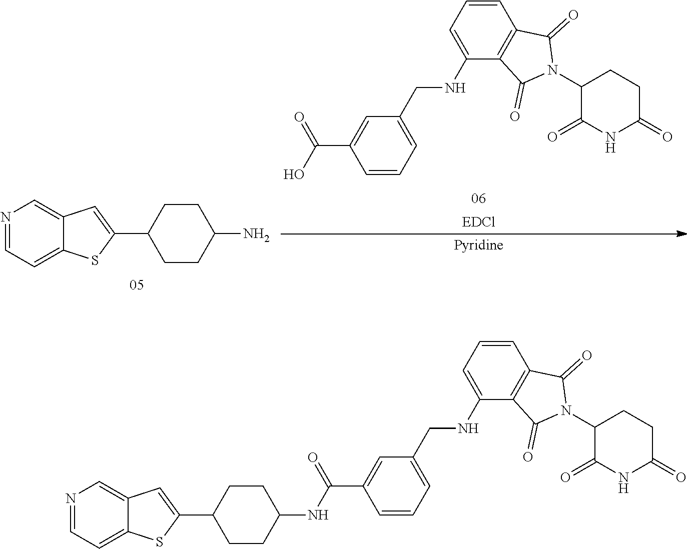 [SMILES: O=C1CCC(N2C(=O)c3cccc(NCc4cccc(C(=O)O)c4)c3C2=O)C(=O)N1 ; NC1CCC(c2cc3cnccc3s2)CC1 ; O=C1CCC(N2C(=O)c3cccc(NCc4cccc(C(=O)NC5CCC(c6cc7cnccc7s6)CC5)c4)c3C2=O)C(=O)N1]

05 (55 mg, 0.237 mmol) was dissolved in pyridine (2 mL) at room temperature, and 06 (96 mg, 0.237 mmol) and EDCI (161 mg, 0.711 mmol) were added. The above mixture was stirred at room temperature for 16 h. The reaction mixture was concentrated to dryness by rotary evaporation, and the residue was purified by preparative high performance liquid chromatography to give the target product as a yellow solid (1.46 mg, 0.99% yield). LC-MS: 622.1 [M+H]⁺, ¹H NMR (400 MHz, DMSO-d₆) δ 11.10 (s, 1H), 9.00 (s, 1H), 8.45-8.42 (m, 1H), 8.34 (s, 2H), 7.97 (s, 1H), 7.88 (s, 1H), 7.75 (s, 1H), 7.52 (s, 2H), 7.49-7.39 (m, 2H), 7.30 (s, 1H), 7.03 (s, 1H), 6.97 (s, 1H), 5.07 (s, 1H), 4.62 (s, 2H), 3.86 (s, 2H), 2.97 (s, 2H), 2.62 (s, 1H), 2.25-1.80 (m, 6H), 1.64-1.47 (s, 3H).

### Example 52

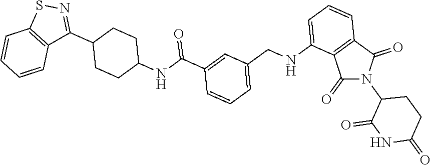 [SMILES: O=C1CCC(N2C(=O)c3cccc(NCc4cccc(C(=O)NC5CCC(c6nsc7ccccc67)CC5)c4)c3C2=O)C(=O)N1]

### (1) Preparation Steps

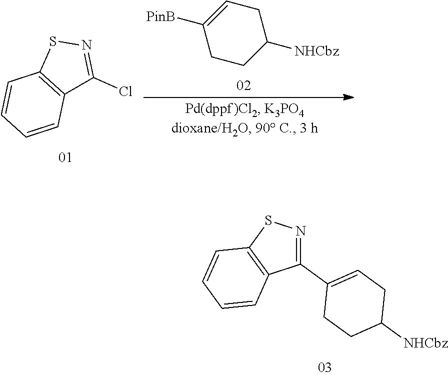

To a 100 mL single-neck flask were added 01 (142.49 mg, 0.84 mmol), 02 (200 mg, 0.56 mmol), potassium phosphate (237.73 mg, 1.12 mmol), tetrahydrofuran (10 mL), water (2 mL) and catalyst Ruphos-Pd (50 mg, 0.0612 mmol). The mixture was reacted in an argon atmosphere at 90° C. for 3 h and concentrated to dryness by rotary evaporation, and the residue was purified by column chromatography to give two products with molecular weight (03-P1: 50 mg, 16.3% yield; 03-P2: 60 mg, 19.6% yield). LC-MS: 365.2 [M+H]⁺. 03-P1:1H NMR (400 MHz, CD3OD) δ 8.24 (d, J=8.2 Hz, 1H), 8.06 (d, J=8.2 Hz, 1H), 7.60 (t, J=7.2 Hz, 1H), 7.51 (t, J=7.3 Hz, 1H), 7.37 (dt, J=22.2, 7.4 Hz, 5H), 6.41 (s, 1H), 5.14 (s, 2H), 3.90 (s, 1H), 2.87 (d, J=16.7 Hz, 1H), 2.72 (d, J=16.7 Hz, 2H), 2.36-2.24 (m, 1H), 2.11 (s, 1H), 1.83 (ddd, J=22.9, 10.3, 5.6 Hz, 1H). 03-P2:1H NMR (400 MHz, CD3OD) δ 7.74 (dd, J=7.7, 1.0 Hz, 1H), 7.65-7.60 (m, 1H), 7.49 (d, J=7.8 Hz, 1H), 7.41 (dd, J=6.6, 5.7 Hz, 1H), 7.39-7.31 (m, 5H), 6.16 (s, 1H), 5.10 (s, 2H), 3.78 (s, 1H), 2.56 (d, J=18.8 Hz, 1H), 2.31 (d, J=16.4 Hz, 1H), 2.28-2.16 (m, 2H), 1.98 (d, J=12.0 Hz, 1H), 1.82-1.69 (m, 1H).

### Step 2

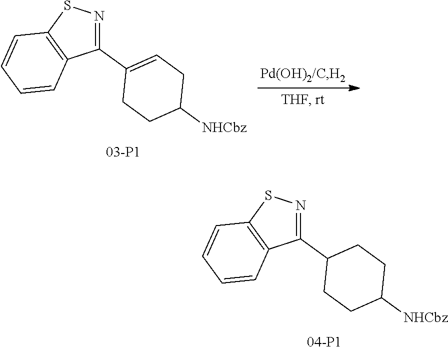 [SMILES: O=C(NC1CC=C(c2nsc3ccccc23)CC1)OCc1ccccc1 ; O=C(NC1CCC(c2nsc3ccccc23)CC1)OCc1ccccc1]

To a 50 mL single-neck flask were added 03-P1 (60 mg, 0.165 mmol), Pd(OH)₂/C (50 mg, 10%) and tetrahydrofuran (10 mL). The mixture was reacted in a hydrogen atmosphere at room temperature for 5 days and filtered, and the filtrate was concentrated to give the target product (04-P1: 18 mg, 29.7% yield). LC-MS: 367.3 [M+H]⁺.

### Step 3

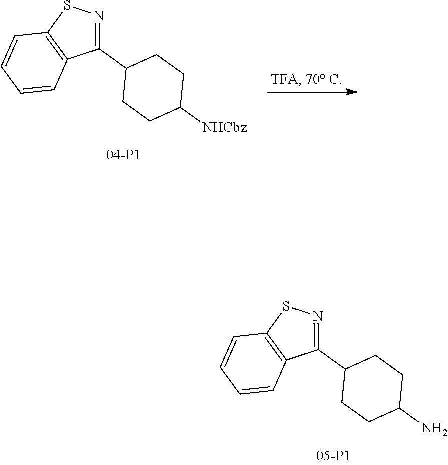 [SMILES: O=C(NC1CCC(c2nsc3ccccc23)CC1)OCc1ccccc1 ; NC1CCC(c2nsc3ccccc23)CC1]

To a 50 mL single-neck flask were added 04-P1 (18 mg, 0.0491 mmol) and trifluoroacetic acid (2 mL), and the mixture was reacted at 70° C. for 1 h and then concentrated to dryness by rotary evaporation to give the target product (05-P1: 10 mg, 87.65% yield). LC-MS: 234.3 [M+H]⁺.

### Step 4

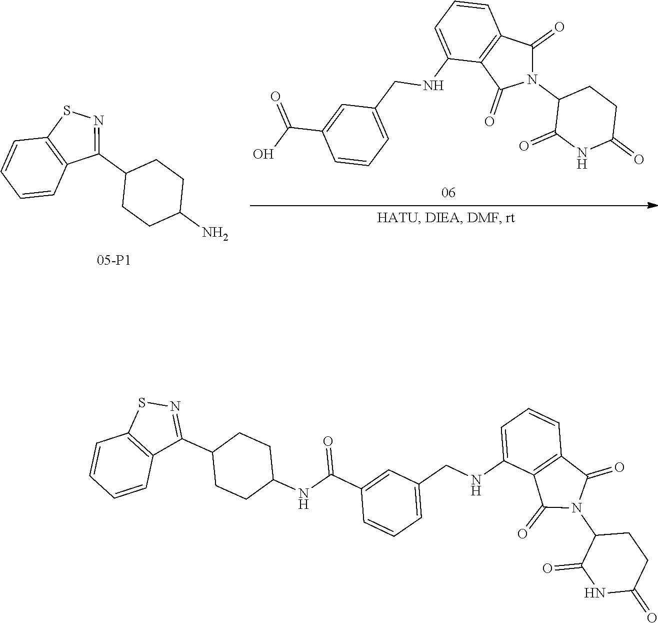 [SMILES: NC1CCC(c2nsc3ccccc23)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6nsc7ccccc67)CC5)c4)c3C2=O)C(=O)N1]

To a 50 mL single-neck flask were added 05-P1 (10 mg, 0.043 mmol), 06 (21.02 mg, 0.0516 mmol), HATU (24.525 mg, 0.0645 mmol), DIEA (16.67 mg, 0.129 mmol) and DMF (2 mL), and the mixture was reacted at room temperature for 2 h. The reaction mixture was purified by preparative high performance liquid chromatography to give a green solid (4.57 mg, 17.1% yield). LC-MS: 622.3 [M+H]⁺.

¹H NMR (400 MHz, DMSO-d6) δ 11.09 (s, 1H), 8.36-8.14 (m, 3H), 7.87 (s, 1H), 7.75 (d, J=7.3 Hz, 1H), 7.60 (t, J=7.4 Hz, 1H), 7.50 (t, J=7.2 Hz, 3H), 7.40 (t, J=7.6 Hz, 1H), 7.29 (d, J=5.5 Hz, 1H), 7.03 (t, J=6.1 Hz, 1H), 6.96 (d, J=8.6 Hz, 1H), 5.11-5.04 (m, 1H), 4.61 (d, J=5.8 Hz, 2H), 3.97 (d, J=69.5 Hz, 1H), 3.55 (s, 1H), 2.87 (d, J=13.4 Hz, 1H), 2.69-2.57 (m, 2H), 2.16-1.71 (m, 9H).

### Example 53

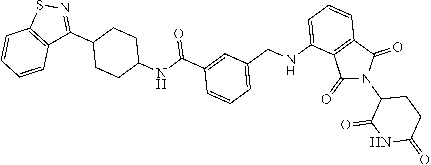 [SMILES: O=C1CCC(N2C(=O)c3cccc(NCc4cccc(C(=O)NC5CCC(c6nsc7ccccc67)CC5)c4)c3C2=O)C(=O)N1]

### Preparation Steps

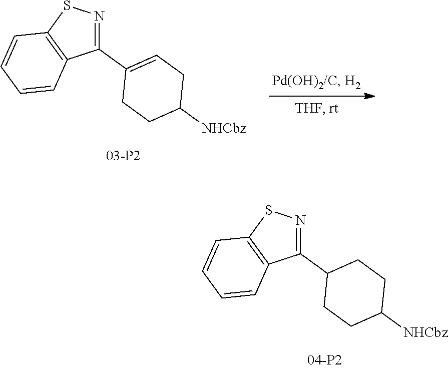 [SMILES: O=C(NC1CC=C(c2nsc3ccccc23)CC1)OCc1ccccc1 ; O=C(NC1CCC(c2nsc3ccccc23)CC1)OCc1ccccc1]

To a 50 mL single-neck flask were added 03-P2 (prepared in Example 2, 50 mg, 0.137 mmol), Pd(OH)₂/C (50 mg) and tetrahydrofuran (10 mL). The mixture was reacted in a hydrogen atmosphere at room temperature for 5 days and filtered, and the filtrate was concentrated to give the target product (04-P2: 12 mg, 23.9% yield). LC-MS: 367.3 [M+H]⁺.

### Step 2

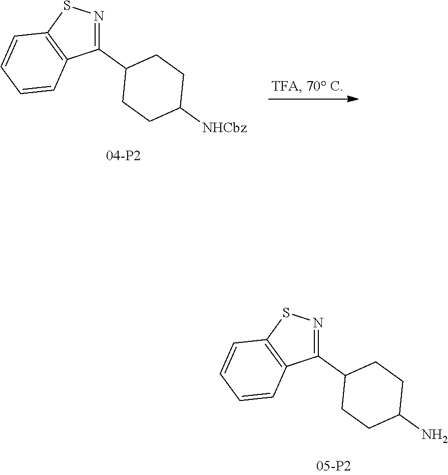 [SMILES: O=C(NC1CCC(c2nsc3ccccc23)CC1)OCc1ccccc1 ; NC1CCC(c2nsc3ccccc23)CC1]

To a 50 mL single-neck flask were added 04-P2 (12 mg, 0.0327 mmol) and trifluoroacetic acid (2 mL), and the mixture was reacted at 70° C. for 1 h and concentrated to dryness by rotary evaporation to give the target product (05-P2: 10 mg, 87.65% yield). LC-MS: 234.3 [M+H]⁺.

### Step 3

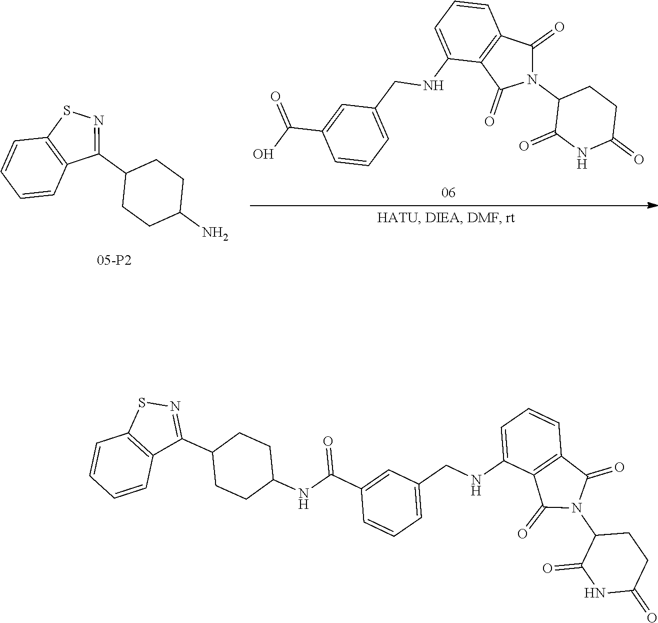 [SMILES: NC1CCC(c2nsc3ccccc23)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6nsc7ccccc67)CC5)c4)c3C2=O)C(=O)N1]

To a 50 mL single-neck flask were added 05-P2 (10 mg, 0.043 mmol), 07 (21.02 mg, 0.0516 mmol), HATU (24.525 mg, 0.0645 mmol), DIEA (16.67 mg, 0.129 mmol) and DMF (2 mL), and the mixture was reacted at room temperature for 2 h. The reaction mixture was purified by preparative high performance liquid chromatography to give a green solid (5.29 mg, 19.8% yield). LC-MS: 622.3 [M+H]⁺.

¹H NMR (400 MHz, DMSO-d6) δ 11.10 (s, 1H), 8.30 (dd, J=34.3, 7.7 Hz, 1H), 7.85 (dd, J=13.0, 6.6 Hz, 2H), 7.77-7.65 (m, 3H), 7.54-7.47 (m, 2H), 7.45-7.37 (m, 2H), 7.29 (d, J=6.3 Hz, 1H), 7.03 (d, J=7.1 Hz, 1H), 6.95 (dd, J=8.5, 4.6 Hz, 1H), 5.07 (dd, J=12.9, 5.4 Hz, 1H), 4.61 (d, J=5.9 Hz, 2H), 4.10-3.56 (m, 2H), 2.95-2.82 (m, 1H), 2.65-2.52 (m, 2H), 2.03 (t, J=12.2 Hz, 2H), 1.93-1.67 (m, 6H), 1.47 (t, J=9.8 Hz, 1H).

### Example 54

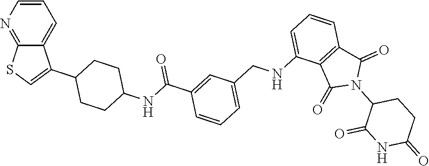 [SMILES: O=C1CCC(N2C(=O)c3cccc(NCc4cccc(C(=O)NC5CCC(c6csc7ncccc67)CC5)c4)c3C2=O)C(=O)N1]

### Preparation Steps

### Step 1

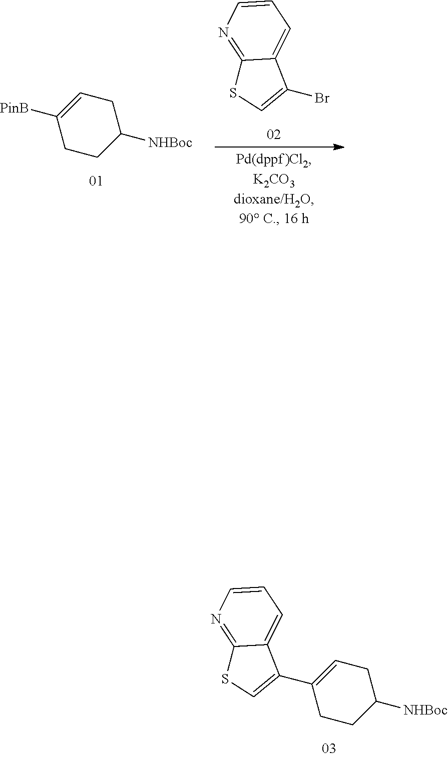

01 (100 mg, 0.310 mmol), 02 (99 mg, 1.464 mmol), [1,1′-bis(diphenylphosphino)ferrocene]palladium dichloride (23 mg, 0.0309 mmol) and potassium carbonate (85 mg, 0.616 mmol) were dissolved in 1,4-dioxane (4 mL) and water (4 mL) at room temperature, and the reaction mixture was stirred in an argon atmosphere at 90° C. for 16 h. Water (50 mL) was added, followed by extraction with ethyl acetate (50 mL×3). The organic phase was washed with saturated brine (50 mL×3), dried over anhydrous sodium sulfate, and filtered, and the filtrate was concentrated to dryness by rotary evaporation to give the target product as a yellow solid (70 mg, 68.6% yield). LC-MS: 331.3 [M+H]⁺.

### Step 2

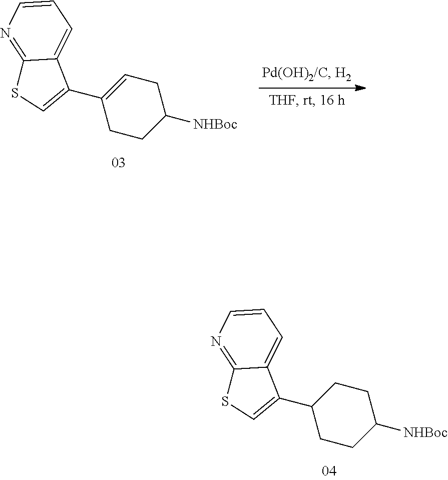 [SMILES: CC(C)(C)OC(=O)NC1CC=C(c2csc3ncccc23)CC1 ; CC(C)(C)OC(=O)NC1CCC(c2csc3ncccc23)CC1]

03 (70 mg, 0.212 mmol) was dissolved in tetrahydrofuran (5 mL) at room temperature, and wet palladium hydroxide on carbon (100 mg) was added. The reaction mixture was purged with hydrogen twice, stirred in a hydrogen atmosphere at room temperature for 16 h, and filtered. The filtrate was concentrated to dryness by rotary evaporation, and the residue was purified through a silica gel column (petroleum ether:ethyl acetate=4:1 to 3:1) to separate the target product as a yellow solid (40 mg, 57.2% yield). LC-MS: 333.2 [M+H]⁺.

### Step 3

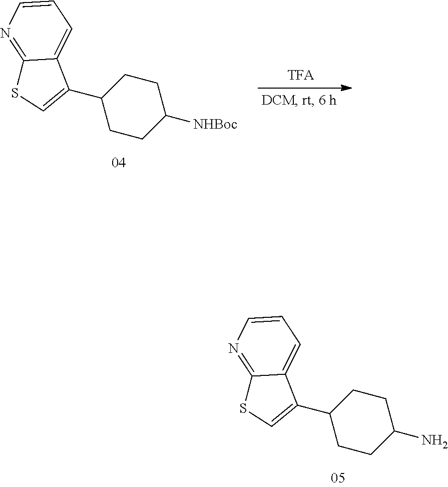 [SMILES: CC(C)(C)OC(=O)NC1CCC(c2csc3ncccc23)CC1 ; NC1CCC(c2csc3ncccc23)CC1]

04 (30 mg, 0.151 mmol) was dissolved in dichloromethane (3 mL) at room temperature, and trifluoroacetic acid (1 mL) was added dropwise with stirring at room temperature. The reaction mixture was stirred at room temperature for 6 h and concentrated by rotary evaporation to remove the solvent to give the target product as a yellow oil (30 mg, crude). LC-MS: 233.2 [M+H]⁺.

### Step 4

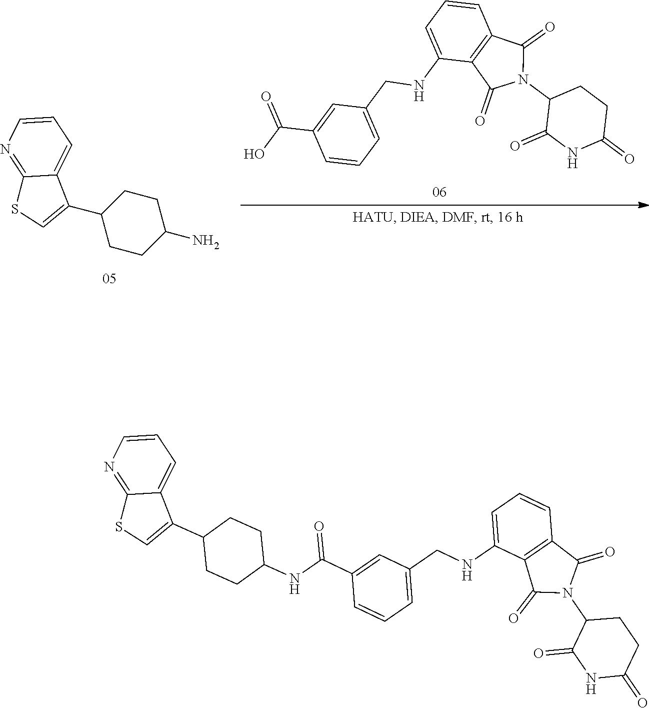 [SMILES: NC1CCC(c2csc3ncccc23)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6csc7ncccc67)CC5)c4)c3C2=O)C(=O)N1]

05 (30 mg, 0.129 mmol), 06 (78 mg, 0.192 mmol), N,N-diisopropylethylamine (48 mg, 0.372 mmol) and 2-(7-azobenzotriazol)-N,N,N,N-tetramethyluronium hexafluorophosphate (144 mg, 0.379 mmol) were dissolved in N,N-dimethylformamide (2 mL) at room temperature, and the reaction mixture was stirred at room temperature for 16 h and purified by preparative chromatography (C18, 5-100% acetonitrile/water as mobile phase, 0.1% formic acid) to separate the target product as a yellow solid (23.79 mg, 29.8% yield). LC-MS: 622.4 [M+H]⁺. ¹H NMR (400 MHz, DMSO-d₆) δ 11.04 (s, 1H), 8.56 (d, J=4.3 Hz, 1H), 8.38-8.10 (m, 2H), 7.92-7.70 (m, 2H), 7.61-7.39 (m, 5H), 7.29 (s, 1H), 7.11-6.92 (m, 2H), 5.08 (dt, J=12.8, 4.7 Hz, 1H), 4.62 (d, J=6.0 Hz, 2H), 4.16 (s, 1H), 3.01-2.82 (m, 2H), 2.58 (dd, J=14.4, 9.6 Hz, 2H), 2.09-1.88 (m, 5H), 1.81 (s, 2H), 1.63 (dd, J=18.8, 10.1 Hz, 2H)

### Example 55

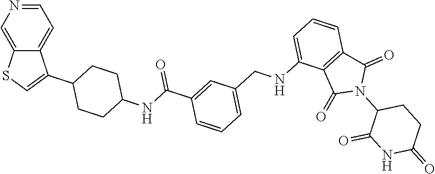 [SMILES: O=C1CCC(N2C(=O)c3cccc(NCc4cccc(C(=O)NC5CCC(c6csc7cnccc67)CC5)c4)c3C2=O)C(=O)N1]

### Preparation Steps

### Step 1

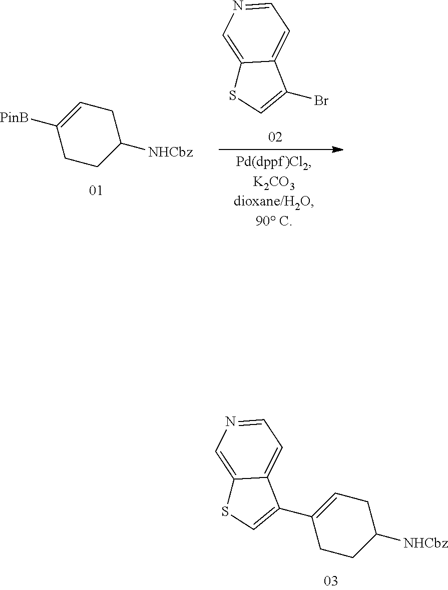

Starting materials 01 (250 mg, 0.7 mmol), 02 (178 mg, 0.84 mmol) and dichloro[1,1′-bis(diphenylphosphino)ferrocene]palladium (52 mg, 0.07 mmol) were dissolved in 1,4-dioxane (4 mL), and a solution of potassium carbonate (242 mg, 1.75 mmol) in water (1 mL) was added. The reaction mixture was then stirred in an argon atmosphere at 90° C. for 3 h. Water (50 mL) was added, followed by extraction with ethyl acetate (50 mL×3). The organic phase was washed with saturated brine (50 mL), dried over anhydrous sodium sulfate, and filtered, and the filtrate was concentrated to dryness by rotary evaporation. The residue was passed through a silica gel column (petroleum ether:ethyl acetate=100:0 to 80:20) to separate the target product as a white solid (170 mg, 67.0% yield). LC-MS: 365.2 [M+H]⁺.

### Step 2

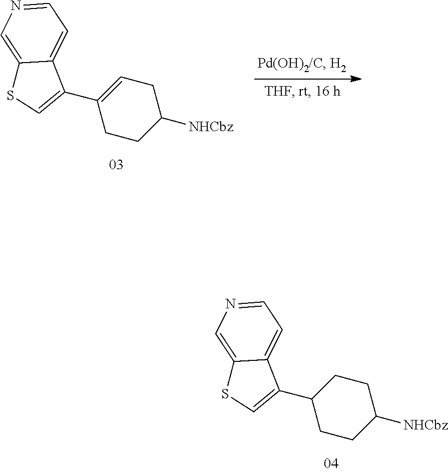 [SMILES: O=C(NC1CC=C(c2csc3cnccc23)CC1)OCc1ccccc1 ; O=C(NC1CCC(c2csc3cnccc23)CC1)OCc1ccccc1]

03 (75 mg, 0.21 mmol) was dissolved in tetrahydrofuran (3 mL) at room temperature, and wet palladium on carbon (40 mg) was added. The mixture was purged twice with hydrogen, stirred in a hydrogen atmosphere at room temperature for 16 h, and filtered, and the filtrate was concentrated to dryness by rotary evaporation to give the target product as a white solid (60 mg, 78.0% yield). LC-MS: 367.2 [M+H]⁺.

### Step 3

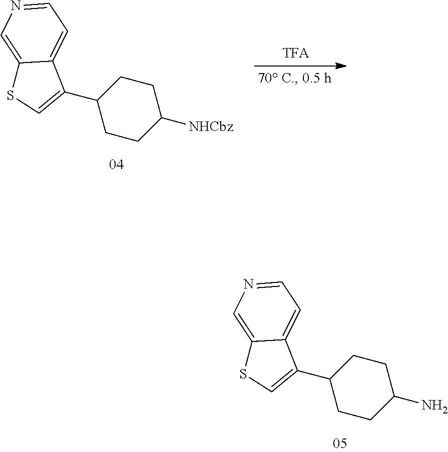 [SMILES: O=C(NC1CCC(c2csc3cnccc23)CC1)OCc1ccccc1 ; NC1CCC(c2csc3cnccc23)CC1]

04 (60 mg, 0.16 mmol) was dissolved in trifluoroacetic acid (1 mL) at room temperature, and the reaction mixture was stirred at 70° C. for 0.5 h and concentrated by rotary evaporation to remove the solvent to give the target product as a yellow oil (36 mg, 95.0% yield). LC-MS: 233.2 [M+H]⁺.

### Step 4

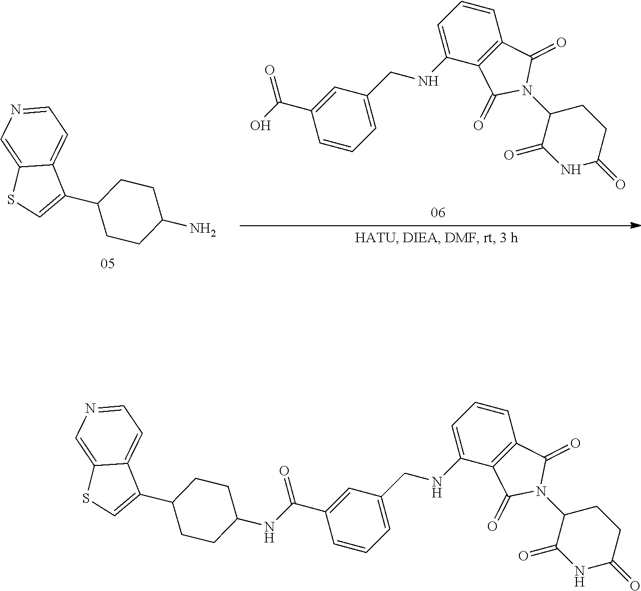 [SMILES: NC1CCC(c2csc3cnccc23)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6csc7cnccc67)CC5)c4)c3C2=O)C(=O)N1]

05 (36 mg, 0.15 mmol), 05 (62 mg, 0.15 mmol) and N,N-diisopropylethylamine (59 mg, 0.45 mmol) were dissolved in N,N-dimethylformamide (0.5 mL) at room temperature, and (7-azabenzotriazole)-tetramethyluronium hexafluorophosphate (69 mg, 0.18 mmol) was added with stirring at room temperature. The reaction mixture was stirred at room temperature for 3 h. The mixture was purified by preparative chromatography (C18, 5-100% acetonitrile/water as mobile phase, 0.1% formic acid) to separate the target product as a yellow solid (14.54 mg, 16% yield). LC-MS: 622.2 [M+H]⁺. ¹H NMR (400 MHz, DMSO-d₆) δ 11.10 (s, 1H), 9.22 (s, 1H), 8.47 (d, J=5.5 Hz, 1H), 8.25 (dd, J=70.3, 7.3 Hz, 1H), 7.91-7.86 (m, 1H), 7.85 (d, J=4.9 Hz, 1H), 7.80 (d, J=10.4 Hz, 1H), 7.74 (t, J=7.5 Hz, 1H), 7.50 (t, J=7.0 Hz, 2H), 7.42 (t, J=7.6 Hz, 1H), 7.30 (t, J=6.0 Hz, 1H), 7.03 (t, J=6.0 Hz, 1H), 6.96 (d, J=8.6 Hz, 1H), 5.11-5.03 (m, 1H), 4.62 (d, J=6.1 Hz, 2H), 4.16 (s, 1H), 3.11 (s, 1H), 2.93-2.82 (m, 1H), 2.66-2.53 (m, 2H), 2.09-1.97 (m, 3H), 1.97-1.88 (m, 2H), 1.86-1.74 (m, 3H), 1.63 (dd, J=18.2, 9.5 Hz, 1H).

### Example 56

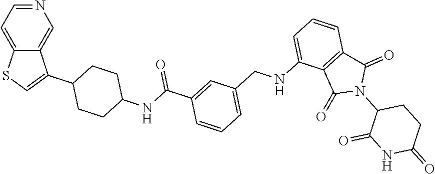 [SMILES: O=C1CCC(N2C(=O)c3cccc(NCc4cccc(C(=O)NC5CCC(c6csc7ccncc67)CC5)c4)c3C2=O)C(=O)N1]

### Preparation Steps

### Step 1

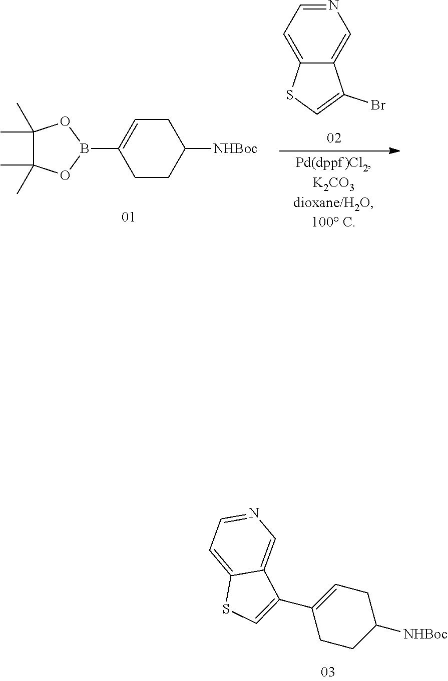 [SMILES: CC(C)(C)OC(=O)NC1CC=C(B2OC(C)(C)C(C)(C)O2)CC1 ; Brc1csc2ccncc12 ; CC(C)(C)OC(=O)NC1CC=C(c2csc3ccncc23)CC1]

01 (181 mg, 0.561 mmol), 02 (120 mg, 0.561 mmol), potassium carbonate (232 mg, 1.68 mmol) and [1,1′-bis(diphenylphosphino)ferrocene]palladium (II) dichloride (41.0 mg, 0.056 mmol) were mixed in dioxane (10 mL) and water (2 mL), and the reaction mixture was purged with argon, stirred in an argon atmosphere at 100° C. for 16 h, cooled to room temperature, and then concentrated under reduced pressure to remove the solvent. Water (100 mL) was added, followed by extraction with ethyl acetate (30 mL×3). The organic phase was washed with saturated brine (60 mL), dried over anhydrous sodium sulfate, and filtered, and the filtrate was concentrated to dryness by rotary evaporation. The residue was purified through a flash silica gel column (petroleum ether:ethyl acetate=1:1) to give the target product as a white solid (130 mg, 70.1% yield). LC-MS: 331.3 [M+H]⁺.

### Step 2

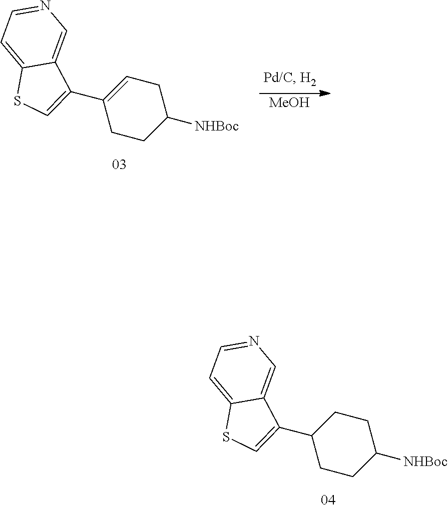 [SMILES: "MeOH" ; CC(C)(C)OC(=O)NC1CC=C(c2csc3ccncc23)CC1 ; CC(C)(C)OC(=O)NC1CCC(c2csc3ccncc23)CC1]

03 (80.0 mg, 0.242 mmol) was dissolved in methanol (2 mL) and tetrahydrofuran (0.5 mL), and 10% palladium on carbon (40.0 mg) was added. The reaction mixture was stirred at room temperature for 16 h and filtered, and the filtrate was concentrated to dryness by rotary evaporation to give the target product as a white solid (37 mg, 46.0% yield). LC-MS: 333.3 [M+H]⁺.

### Step 3

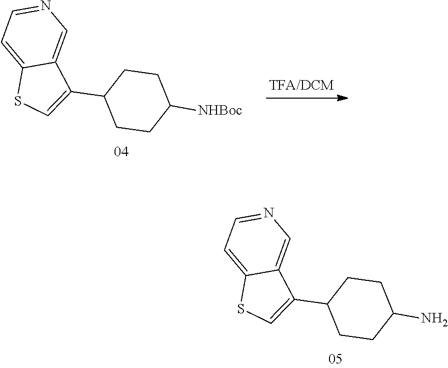 [SMILES: CC(C)(C)OC(=O)NC1CCC(c2csc3ccncc23)CC1 ; NC1CCC(c2csc3ccncc23)CC1]

04 (37.0 mg, 0.111 mmol) was dissolved in dichloromethane (2 mL), and then trifluoroacetic acid (2 mL) was added. The reaction mixture was stirred at room temperature for 2 h and concentrated to dryness by rotary evaporation to give the target product as a colorless oil (26 mg, crude). LC-MS: 233.3 [M+H]⁺.

### Step 4

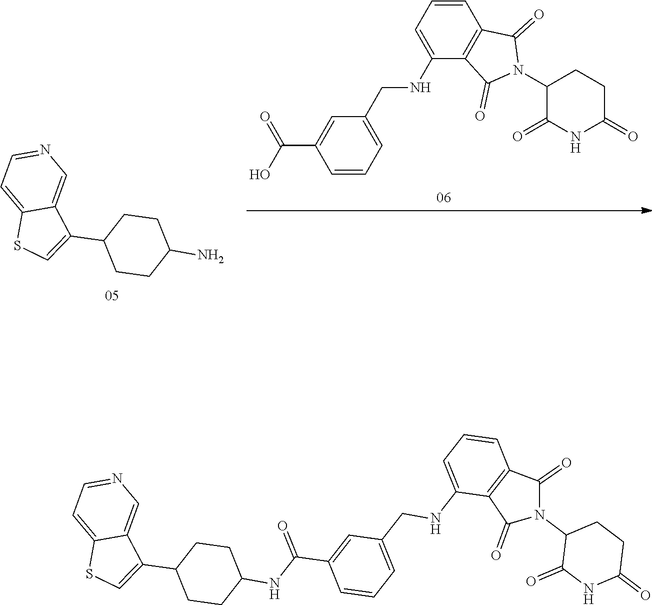 [SMILES: NC1CCC(c2csc3ccncc23)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6csc7ccncc67)CC5)c4)c3C2=O)C(=O)N1]

05 (26.0 mg, 0.111 mmol), 06 (45.2 mg, 0.111 mmol) and N,N-diisopropylethylamine (57.4 mg, 0.444 mmol) were mixed in anhydrous N,N-dimethylformamide (1 mL) at room temperature, and (7-azabenzotriazole)-tetramethyluronium hexafluorophosphate (63.3 mg, 0.167 mmol) was added in batches with stirring at room temperature. The reaction mixture was stirred at room temperature for 1 h. The mixture was purified by preparative chromatography (C18, 5-100% acetonitrile/water as mobile phase, 0.1% formic acid) to separate the target product as a yellow solid (32.74 mg, 47.0% yield over two steps). LC-MS: 622.2 [M+H]⁺, ¹H NMR (400 MHz, DMSO-d₆) δ 11.11 (s, 1H), 9.32-9.23 (m, 1H), 8.52-8.31 (m, 2H), 8.19-8.15 (m, 1H), 7.91-7.82 (m, 1H), 7.79-7.71 (m, 1H), 7.66-7.59 (m, 1H), 7.54-7.48 (m, 2H), 7.45-7.39 (m, 1H), 7.34-7.26 (m, 1H), 7.06-7.00 (m, 1H), 6.98-6.93 (m, 1H), 5.14-5.02 (m, 1H), 4.62 (d, J=6.2 Hz, 2H), 4.20-3.86 (m, 1H), 3.15-3.06 (m, 1H), 2.94-2.83 (m, 1H), 2.65-2.54 (m, 2H), 2.10-1.76 (m, 7H), 1.72-1.58 (m, 2H).

### Example 57

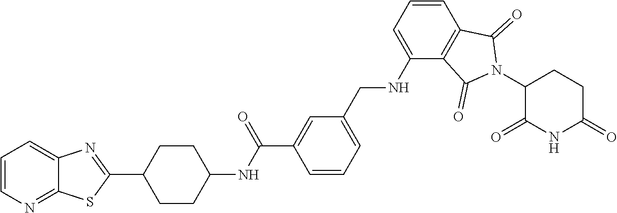 [SMILES: O=C1CCC(N2C(=O)c3cccc(NCc4cccc(C(=O)NC5CCC(c6nc7cccnc7s6)CC5)c4)c3C2=O)C(=O)N1]

### Preparation Steps

### Step 1

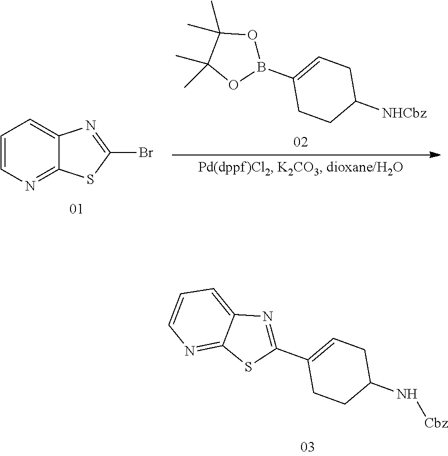 [SMILES: Brc1nc2cccnc2s1 ; CC1(C)OB(C2=CCC(NC(=O)OCc3ccccc3)CC2)OC1(C)C ; O=C(NC1CC=C(c2nc3cccnc3s2)CC1)OCc1ccccc1]

In a 100 mL single-neck flask, 01 (150 mg, 0.699 mmol) was dissolved in 1,4-dioxane (10 mL) and water (2 mL), and 02 (208 mg, 0.582 mmol), potassium carbonate (241 mg, 1.75 mmol) and Pd(dppf)Cl₂ (42 mg, 0.0582 mmol) were added. The mixture was reacted in an argon atmosphere at 80° C. for 16 h, cooled to room temperature, quenched with water (10 mL), and extracted with dichloromethane (25 mL×3). The organic phases were combined, washed with saturated brine (15 mL×3), dried over anhydrous sodium sulfate, and filtered, and the filtrate was concentrated. The residue was purified by column chromatography (dichloromethane:methanol=20:1) to give the target product as a white solid (120 mg, 56.4% yield). LC-MS: 366.1 [M+H]⁺.

### Step 2

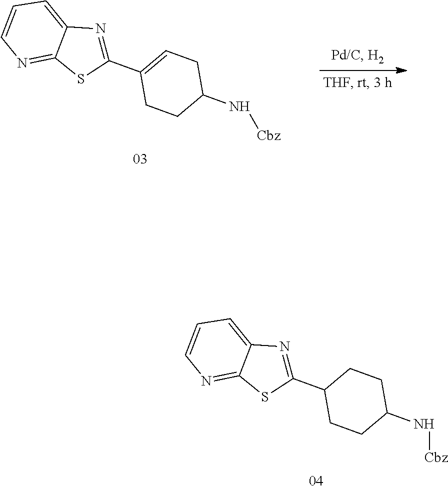 [SMILES: O=C(NC1CC=C(c2nc3cccnc3s2)CC1)OCc1ccccc1 ; O=C(NC1CCC(c2nc3cccnc3s2)CC1)OCc1ccccc1]

In a 100 mL single-neck flask, 03 (60 mg, 0.164 mmol) was dissolved in tetrahydrofuran (10 mL), and palladium on carbon (20 mg) was added. The reaction mixture was stirred in a hydrogen atmosphere for 16 h and filtered, and the filtrate was concentrated to dryness by rotary evaporation to give the target product as a yellow oil (45 mg, 74.7% yield). LC-MS: 368.1 [M+H]⁺.

### Step 3

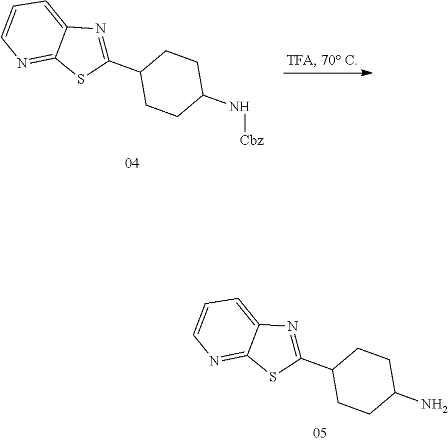 [SMILES: O=C(NC1CCC(c2nc3cccnc3s2)CC1)OCc1ccccc1 ; NC1CCC(c2nc3cccnc3s2)CC1]

In a 50 mL single-neck flask, 04 (45 mg, 0.123 mmol) was dissolved in trifluoroacetic acid (1 mL), and the mixture was heated to 70° C., reacted for 2 h, and concentrated to dryness by rotary evaporation to give the target crude product as a yellow oil (30 mg, crude). LC-MS: 234.1 [M+H]⁺.

### Step 4

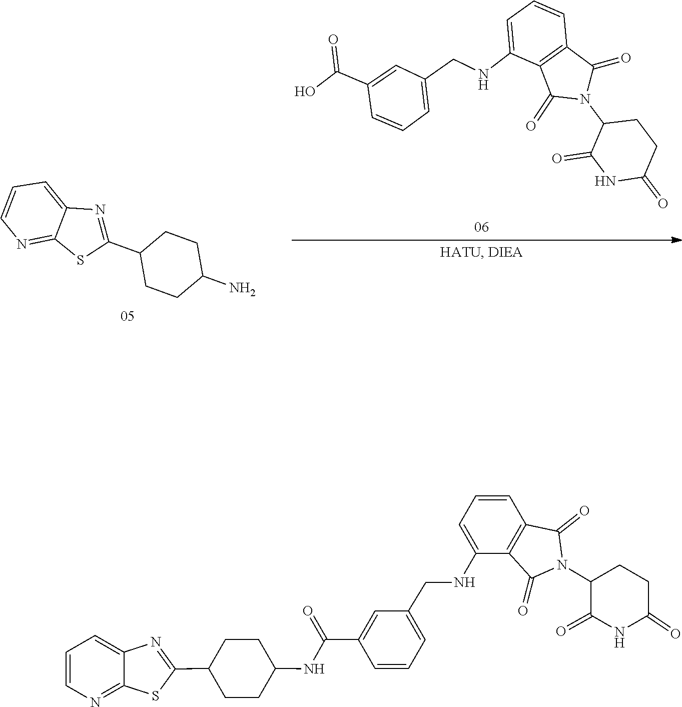 [SMILES: NC1CCC(c2nc3cccnc3s2)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6nc7cccnc7s6)CC5)c4)c3C2=O)C(=O)N1]

05 (30 mg, 0.123 mmol) was dissolved in N,N-dimethylformamide (0.5 mL) at room temperature, and HATU (70 mg, 0.185 mmol), N,N-diisopropylethylamine (50 mg, 0.386 mmol) and 04 (50 mg, 0.386 mmol) were added successively. The above mixture was stirred at room temperature for 2 h. The reaction mixture was purified by preparative high performance liquid chromatography (FA) to give the target product as a yellow solid (33.06 mg, 43.2% yield). LC-MS: 623.3 [M+H]⁺.

¹H NMR (400 MHz, DMSO-d₆) δ 11.10 (s, 1H), 8.58 (d, J=4.5 Hz, 1H), 8.33 (t, J=8.6 Hz, 2H), 7.85 (d, J=18.1 Hz, 1H), 7.79-7.71 (m, 1H), 7.56 (dd, J=8.1, 4.7 Hz, 1H), 7.51 (d, J=7.5 Hz, 1H), 7.41 (dd, J=17.0, 9.2 Hz, 1H), 7.29 (s, 1H), 7.22-7.07 (m, 1H), 7.02 (t, J=8.0 Hz, 1H), 6.94 (t, J=10.2 Hz, 1H), 5.08 (dd, J=12.9, 5.2 Hz, 1H), 4.62 (d, J=5.7 Hz, 2H), 3.92 (d, J=32.7 Hz, 1H), 3.15 (t, J=11.7 Hz, 1H), 2.90 (t, J=13.0 Hz, 1H), 2.62-2.55 (m, 1H), 2.35-2.14 (m, 3H), 2.08-1.98 (m, 3H), 1.78-1.69 (m, 2H), 1.64-1.50 (m, 2H).

### Example 58

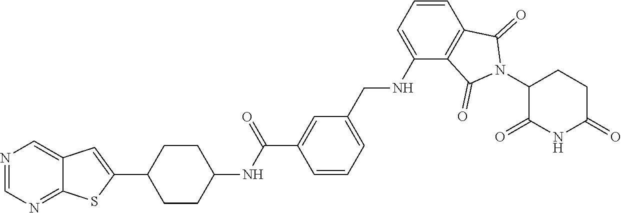 [SMILES: O=C1CCC(N2C(=O)c3cccc(NCc4cccc(C(=O)NC5CCC(c6cc7cncnc7s6)CC5)c4)c3C2=O)C(=O)N1]

### Preparation Steps

### Step 1

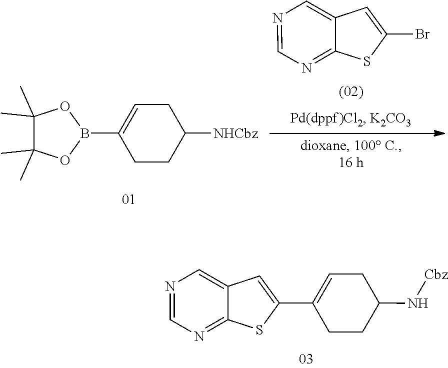 [SMILES: Brc1cc2cncnc2s1 ; CC1(C)OB(C2=CCC(NC(=O)OCc3ccccc3)CC2)OC1(C)C ; O=C(NC1CC=C(c2cc3cncnc3s2)CC1)OCc1ccccc1]

To a 100 mL single-neck flask were added 01 (200 mg, 0.57 mmol), 02 (120 mg, 0.57 mmol), potassium carbonate (232 mg, 1.67 mmol), Pd(dppf)Cl₂ (40 mg, 0.057 mmol), dioxane (4 mL) and water (1 mL), and the reaction mixture was stirred in a nitrogen atmosphere at 100° C. for 16 h, cooled to room temperature, and concentrated to dryness by rotary evaporation to give a crude product, which was then purified by Pre-TLC (PE:EA=1:3) to give the target product as a brown solid (180 mg, 88% yield). LC-MS: 366.0 [M+H]⁺.

### Step 2

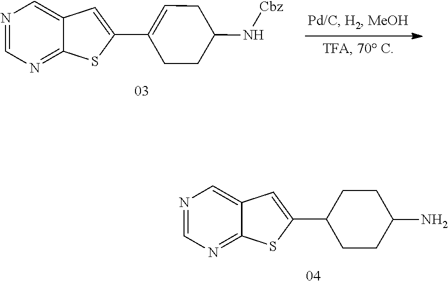 [SMILES: O=C(NC1CC=C(c2cc3cncnc3s2)CC1)OCc1ccccc1 ; NC1CCC(c2cc3cncnc3s2)CC1]

In a 100 mL single-neck flask, 03 (100 mg, 0.27 mmol) was dissolved in methanol (3 mL), and palladium on carbon (10 mg) was added. The reaction mixture was stirred in a hydrogen atmosphere for 16 h and filtered, and the filtrate was concentrated to dryness by rotary evaporation. Trifluoroacetic acid (2 mL) was added, and the mixture was heated at reflux at 70° C. for 3 h and concentrated to dryness by rotary evaporation to give the target product as a yellow oil (30 mg, 47.7% yield), which was directly used in the next step without purification. LC-MS: 234.1 [M+H]⁺.

### Step 3

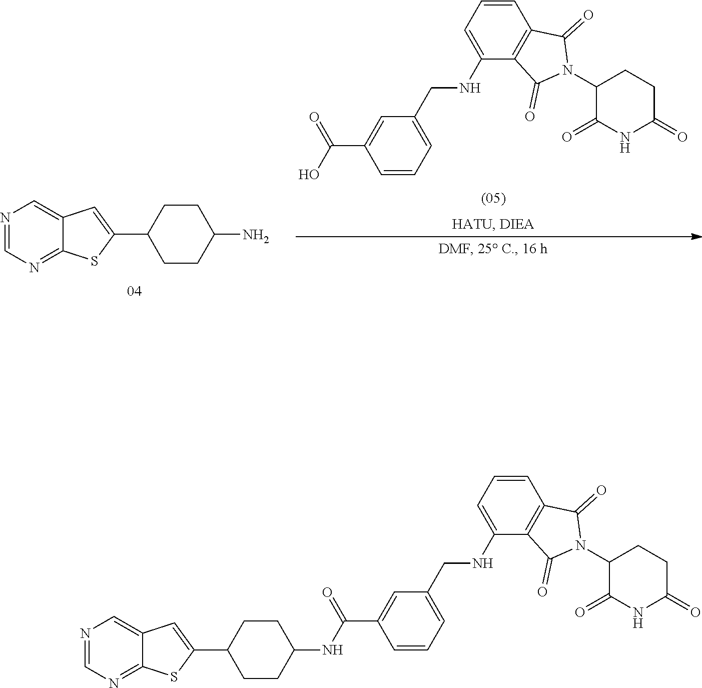 [SMILES: O=C1CCC(N2C(=O)c3cccc(NCc4cccc(C(=O)O)c4)c3C2=O)C(=O)N1 ; NC1CCC(c2cc3cncnc3s2)CC1 ; O=C1CCC(N2C(=O)c3cccc(NCc4cccc(C(=O)NC5CCC(c6cc7cncnc7s6)CC5)c4)c3C2=O)C(=O)N1]

04 (30 mg, 0.128 mmol) was dissolved in DMF (2 mL) at room temperature, and 05 (52 mg, 0.128 mmol), HATU (72 mg, 0.192 mmol) and DIEA (48 mg, 0.384 mmol) were added. The mixture was stirred at room temperature for 16 h. The reaction mixture was purified by preparative high performance liquid chromatography to give the target product as a yellow solid (11.84 mg, 14.9% yield). LC-MS: 623 [M+H]⁺, ¹H NMR (400 MHz, DMSO-d₆) δ 11.10 (s, 1H), 9.19 (d, J=4.3 Hz, 1H), 9.01 (s, 1H), 8.30-8.20 (m, 1H), 7.98-7.60 (m, 2H), 7.60-7.23 (m, 5H), 6.98 (dt, J=16.5, 7.5 Hz, 2H), 5.06 (s, 1H), 4.61 (s, 2H), 3.21 (s, 1H), 2.89 (s, 1H), 2.64-2.53 (m, 2H), 2.33 (s, 1H), 2.17-1.80 (m, 5H), 1.76 (s, 2H), 1.68-1.47 (m, 2H).

### Example 59

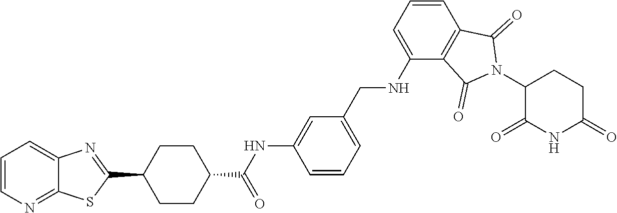 [SMILES: O=C1CCC(N2C(=O)c3cccc(NCc4cccc(NC(=O)[C@H]5CC[C@H](c6nc7cccnc7s6)CC5)c4)c3C2=O)C(=O)N1]

### Preparation Steps

### Step 1

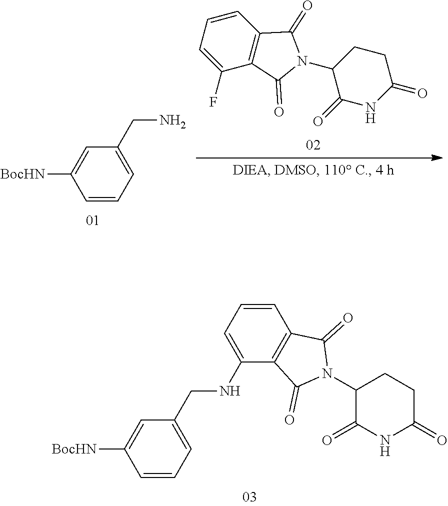 [SMILES: CC(C)(C)OC(=O)Nc1cccc(CN)c1 ; O=C1CCC(N2C(=O)c3cccc(F)c3C2=O)C(=O)N1 ; CC(C)(C)OC(=O)Nc1cccc(CNc2cccc3c2C(=O)N(C2CCC(=O)NC2=O)C3=O)c1]

01 (100 mg, 0.450 mmol), 02 (124 mg, 0.450 mmol) and N,N-diisopropylethylamine (174 mg, 1.35 mmol) were dissolved in anhydrous dimethyl sulfoxide (1 mL) at room temperature, and the reaction mixture was stirred at 110° C. for 4 h. The mixture was purified through a reversed-phase column (C18, 10-100% acetonitrile/water as mobile phase, 0.1% formic acid) to give the target product as a yellow solid (80.0 mg, 37.2% yield). LC-MS: 379.3 [M+H−100]⁺.

### Step 2

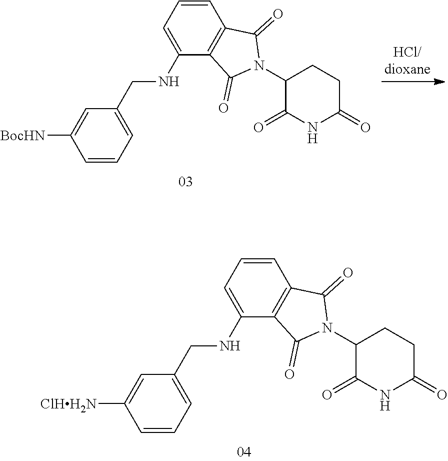

03 (80.0 mg, 0.167 mmol) was dissolved in dichloromethane (1 mL) at room temperature, and a solution of hydrogen chloride in dioxane (1 mL) was added dropwise. The reaction mixture was stirred at room temperature for 16 h and concentrated to dryness by rotary evaporation to give the target product as a yellow solid (80.0 mg, crude). LC-MS: 379.4 [M+H]⁺.

### Step 3

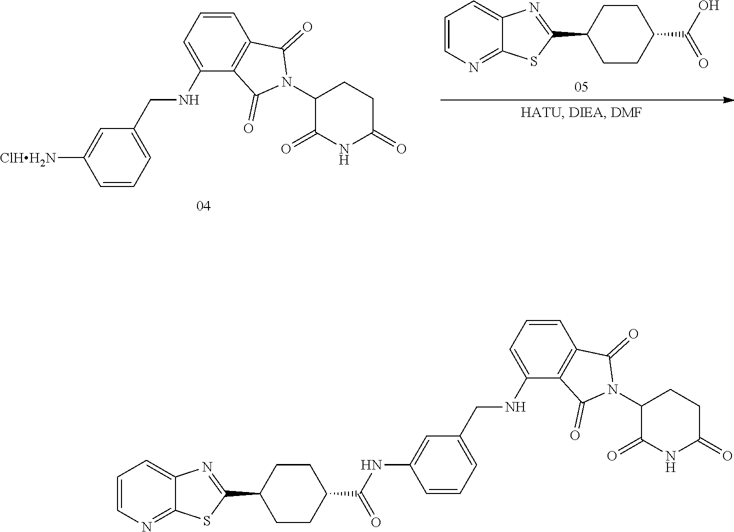

04 (70.0 mg, 0.169 mmol), 05 (44.3 mg, 0.169 mmol) and N,N-diisopropylethylamine (65.5 mg, 0.507 mmol) were mixed in anhydrous N,N-dimethylformamide (2 mL) at room temperature, and (7-azabenzotriazole)-tetramethyluronium hexafluorophosphate (96.6 mg, 0.254 mmol) was added in batches with stirring at room temperature. The reaction mixture was stirred at room temperature for 2 h. The mixture was purified by preparative chromatography (C18, 5-100% acetonitrile/water as mobile phase, 0.03% trifluoroacetic acid) to separate the target product as a yellow solid (21.52 mg, 20.5% yield). LC-MS: 623.2 [M+H]⁺, ¹H NMR (400 MHz, DMSO) δ 11.03 (s, 1H), 9.84 (s, 1H), 8.50 (dd, J=4.6, 1.4 Hz, 1H), 8.26 (dd, J=8.2, 1.4 Hz, 1H), 7.53-7.42 (m, 4H), 7.21-7.13 (m, 2H), 6.96 (d, J=7.1 Hz, 2H), 6.84 (d, J=8.6 Hz, 1H), 5.00 (dd, J=13.0, 5.3 Hz, 1H), 4.46 (d, J=5.6 Hz, 2H), 3.14-3.07 (m, 1H), 2.87-2.77 (m, 1H), 2.56-2.45 (m, 2H), 2.37-2.29 (m, 1H), 2.21-2.14 (m, 2H), 1.99-1.86 (m, 3H), 1.63-1.50 (m, 4H).

### Example 60

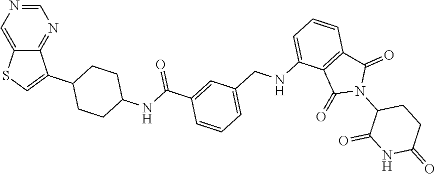 [SMILES: O=C1CCC(N2C(=O)c3cccc(NCc4cccc(C(=O)NC5CCC(c6csc7cncnc67)CC5)c4)c3C2=O)C(=O)N1]

### Preparation Steps

### Step 1

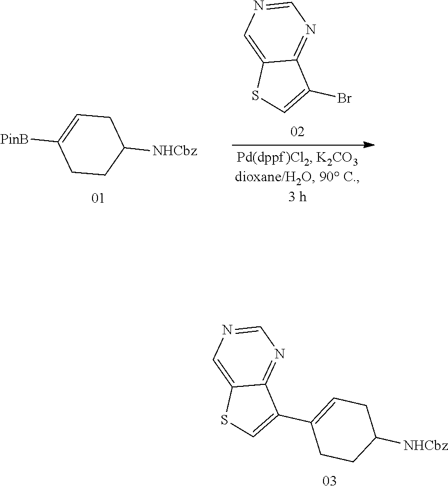

Starting materials 01 (150 mg, 0.42 mmol), 02 (108 mg, 0.5 mmol) and dichloro[1,1′-bis(diphenylphosphino)ferrocene]palladium (30 mg, 0.04 mmol) were dissolved in 1,4-dioxane (4 mL). Potassium carbonate (145 mg, 1.05 mmol) was dissolved in water (1 mL), and the resulting solution was added to the above mixture. The reaction mixture was then stirred at 90° C. for 3 h. Water (50 mL) was added, followed by extraction with ethyl acetate (50 mL×3). The organic phase was washed with saturated brine (50 mL), dried over anhydrous sodium sulfate, and filtered, and the filtrate was concentrated to dryness by rotary evaporation. The residue was passed through a silica gel column (petroleum ether:ethyl acetate=100:0 to 60:40) to separate the target product as a white solid (96 mg, 63.0% yield). LC-MS: 366.3 [M+H]⁺.

### Step 2

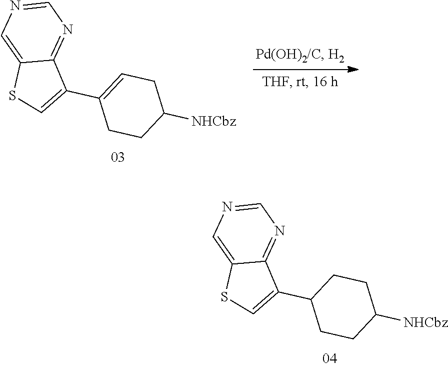 [SMILES: O=C(NC1CC=C(c2csc3cncnc23)CC1)OCc1ccccc1 ; O=C(NC1CCC(c2csc3cncnc23)CC1)OCc1ccccc1]

03 (96 mg, 0.26 mmol) was dissolved in tetrahydrofuran (3 mL) at room temperature, and wet palladium on carbon (50 mg) was added. The mixture was purged twice with hydrogen, stirred in a hydrogen atmosphere at room temperature for 16 h, and filtered, and the filtrate was concentrated to dryness by rotary evaporation to give the target product as a white solid (86 mg, 89.0% yield). LC-MS: 368.2 [M+H]⁺.

### Step 3

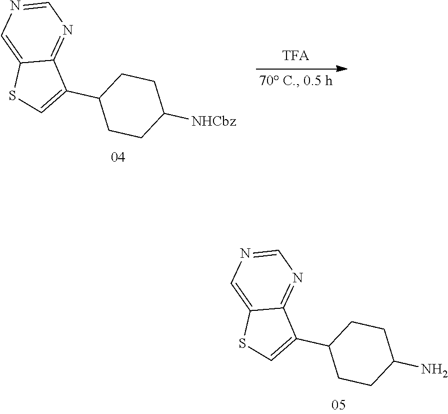 [SMILES: O=C(NC1CCC(c2csc3cncnc23)CC1)OCc1ccccc1 ; NC1CCC(c2csc3cncnc23)CC1]

04 (86 mg, 0.23 mmol) was dissolved in trifluoroacetic acid (1 mL) at room temperature, and the reaction mixture was stirred at 70° C. for 0.5 h and concentrated by rotary evaporation to remove the solvent to give the target product as a yellow oil (50 mg, 93.0% yield). LC-MS: 234.4 [M+H]⁺.

### Step 4

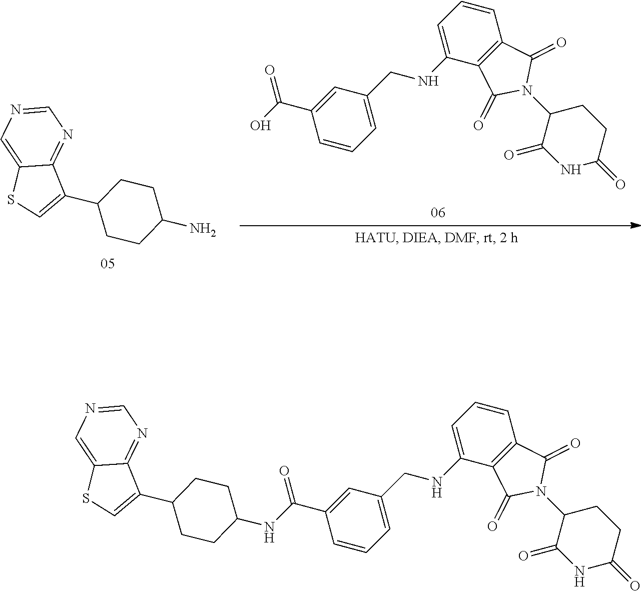 [SMILES: NC1CCC(c2csc3cncnc23)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6csc7cncnc67)CC5)c4)c3C2=O)C(=O)N1]

05 (50 mg, 0.21 mmol), 05 (88 mg, 0.21 mmol) and N,N-diisopropylethylamine (82 mg, 0.63 mmol) were dissolved in N,N-dimethylformamide (1.0 mL) at room temperature, and (7-azabenzotriazole)-tetramethyluronium hexafluorophosphate (96 mg, 0.25 mmol) was added with stirring at room temperature. The reaction mixture was stirred at room temperature for 2 h. The mixture was purified by preparative chromatography (C18, 5-100% acetonitrile/water as mobile phase, 0.1% formic acid) to separate the target product as a yellow solid (38.57 mg, 30% yield). LC-MS: 623.2 [M+H]⁺. ¹H NMR (400 MHz, DMSO-d₆) δ 11.09 (s, 1H), 9.51 (s, 1H), 9.19 (d, J=3.6 Hz, 1H), 8.33-8.11 (m, 2H), 7.86 (d, J=16.7 Hz, 1H), 7.77-7.72 (m, 1H), 7.54-7.48 (m, 2H), 7.42 (t, J=7.6 Hz, 1H), 7.29 (s, 1H), 7.03 (dd, J=6.9, 5.3 Hz, 1H), 6.96 (dd, J=8.6, 1.9 Hz, 1H), 5.11-5.04 (m, 1H), 4.62 (d, J=4.5 Hz, 2H), 4.13 (s, 1H), 3.29-3.08 (m, 1H), 2.95-2.84 (m, 1H), 2.63-2.55 (m, 2H), 2.12-1.97 (m, 4H), 1.88 (d, J=8.6 Hz, 2H), 1.78-1.65 (m, 2H), 1.57 (dd, J=22.5, 11.8 Hz, 1H).

### Example 61

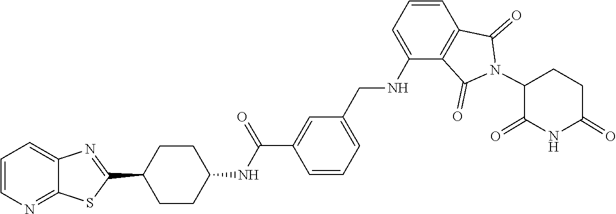 [SMILES: O=C1CCC(N2C(=O)c3cccc(NCc4cccc(C(=O)N[C@H]5CC[C@H](c6nc7cccnc7s6)CC5)c4)c3C2=O)C(=O)N1]

### Step 1

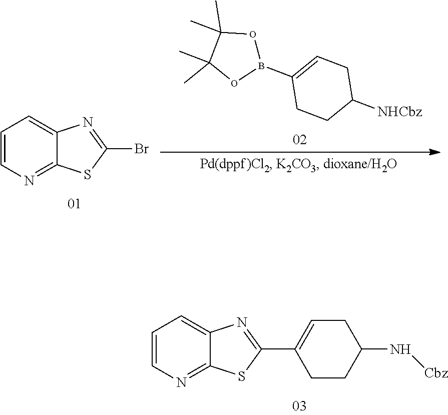 [SMILES: Brc1nc2cccnc2s1 ; CC1(C)OB(C2=CCC(NC(=O)OCc3ccccc3)CC2)OC1(C)C ; O=C(NC1CC=C(c2nc3cccnc3s2)CC1)OCc1ccccc1]

In a 100 mL single-neck flask, 01 (100 mg, 0.465 mmol) was dissolved in 1,4-dioxane (5 mL) and water (1 mL), and 02 (166 mg, 0.465 mmol), potassium carbonate (193 mg, 1.39 mmol) and Pd(dppf)Cl₂ (34 mg, 0.0465 mmol) were added. The mixture was reacted in an argon atmosphere at 100° C. for 16 h, cooled to room temperature, quenched with water (10 mL), and extracted with dichloromethane (25 mL×3). The organic phases were combined, washed with saturated brine (15 mL×3), dried over anhydrous sodium sulfate, and filtered, and the filtrate was concentrated. The residue was purified by column chromatography (dichloromethane:methanol=20:1) to give 03 (155 mg, 91.2% yield, pale brown solid). LC-MS: 366.3 [M+H]⁺.

### Step 2

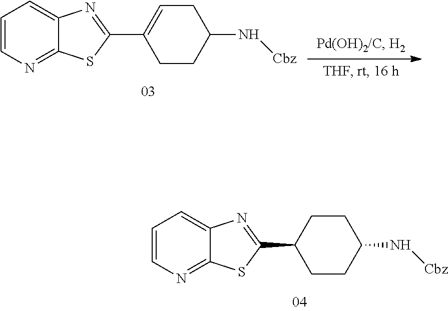 [SMILES: O=C(NC1CC=C(c2nc3cccnc3s2)CC1)OCc1ccccc1 ; O=C(N[C@H]1CC[C@H](c2nc3cccnc3s2)CC1)OCc1ccccc1]

In a 100 mL single-neck flask, 03 (155 mg, 0.424 mmol) was dissolved in tetrahydrofuran (15 mL), and palladium on carbon (30 mg) was added. The reaction mixture was stirred in a hydrogen atmosphere for 16 h and filtered, and the filtrate was concentrated to dryness by rotary evaporation. The residue was purified by column chromatography (petroleum ether:ethyl acetate=3:1) to give 04-F1 (65 mg, 41.7% yield, transparent oil) and 04-F2 (56 mg, 35.9% yield, white solid). The 04-F2 has a trans structure by nuclear magnetic identification.

LC-MS: 368.1 [M+H]⁺.

### Step 3

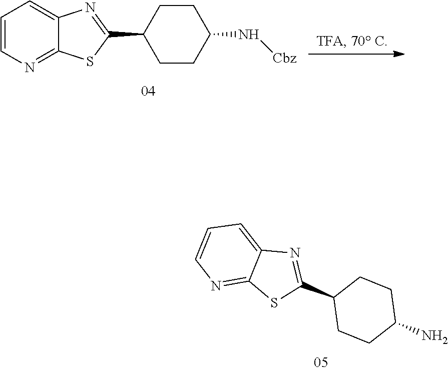 [SMILES: O=C(N[C@H]1CC[C@H](c2nc3cccnc3s2)CC1)OCc1ccccc1 ; N[C@H]1CC[C@H](c2nc3cccnc3s2)CC1]

In a 50 mL single-neck flask, 04-F2 (56 mg, 0.153 mmol) was dissolved in trifluoroacetic acid (1 mL), and the mixture was heated to 70° C., reacted for 2 h, and concentrated to dryness by rotary evaporation to give crude 05 (60 mg, crude, yellow oil).

LC-MS: 234.0 [M+H]⁺.

### Step 4

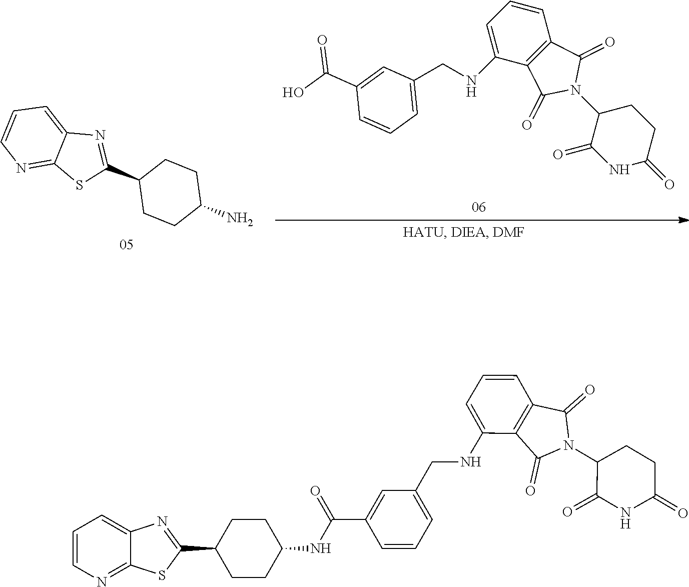 [SMILES: N[C@H]1CC[C@H](c2nc3cccnc3s2)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)N[C@H]5CC[C@H](c6nc7cccnc7s6)CC5)c4)c3C2=O)C(=O)N1]

Crude 05 (30 mg, 0.0765 mmol) was dissolved in N,N-dimethylformamide (1 mL) at room temperature, and HATU (44 mg, 0.115 mmol), N,N-diisopropylethylamine (30 mg, 0.230 mmol) and 04 (31 mg, 0.0765 mmol) were added successively. The above mixture was stirred at room temperature for 2 h. The reaction mixture was purified by preparative high performance liquid chromatography (FA) to give the target product (28.82 mg, 60.8% yield, yellow solid). LC-MS: 623.3 [M+H]⁺.

¹H NMR (400 MHz, DMSO-d₆) δ 11.10 (s, 1H), 8.61-8.53 (m, 1H), 8.34 (dd, J=18.3, 8.7 Hz, 2H), 7.86 (d, J=18.4 Hz, 1H), 7.72 (dd, J=20.8, 7.5 Hz, 1H), 7.58-7.53 (m, 1H), 7.51 (d, J=7.9 Hz, 1H), 7.42 (t, J=7.6 Hz, 1H), 7.29 (t, J=6.0 Hz, 1H), 7.02 (t, J=7.9 Hz, 1H), 6.95 (t, J=10.2 Hz, 1H), 5.08 (dd, J=12.8, 5.4 Hz, 1H), 4.60 (dd, J=13.9, 6.3 Hz, 2H), 4.05-3.81 (m, 1H), 3.15 (t, J=11.9 Hz, 1H), 2.97-2.80 (m, 1H), 2.65-2.54 (m, 2H), 2.25 (d, J=12.2 Hz, 2H), 2.04 (dd, J=20.4, 8.4 Hz, 3H), 1.74 (dd, J=23.7, 11.1 Hz, 2H), 1.57 (dd, J=23.2, 11.0 Hz, 2H).

### Example 62

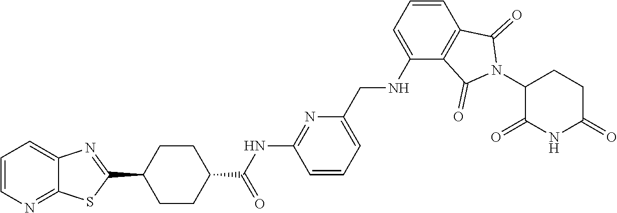 [SMILES: O=C1CCC(N2C(=O)c3cccc(NCc4cccc(NC(=O)[C@H]5CC[C@H](c6nc7cccnc7s6)CC5)n4)c3C2=O)C(=O)N1]

### Preparation Steps

### Step 1

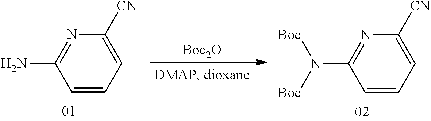 [SMILES: "Boc2O" ; N#Cc1cccc(N)n1 ; CC(C)(C)OC(=O)N(C(=O)OC(C)(C)C)c1cccc(C#N)n1]

Starting materials 01 (500 mg, 4.201 mmol), Boc anhydride (2015 mg, 9.243 mmol) and 4-dimethylaminopyridine (513 mg, 4.201 mmol) were dissolved in 1,4-dioxane (10 mL) at room temperature, and the reaction mixture was stirred at room temperature for 3 h. Water (100 mL) was added, followed by extraction with ethyl acetate (200 mL×3). The organic phase was washed with saturated brine (100 mL), dried over anhydrous sodium sulfate, and filtered, and the filtrate was concentrated to dryness by rotary evaporation. The residue was purified through a silica gel column (petroleum ether:ethyl acetate=4:1 to 3:1) to give the target product as a white solid (800 mg, 59.7%). LC-MS: 320.2 [M+H]⁺.

### Step 2

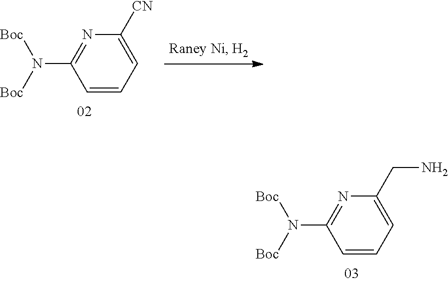 [SMILES: CC(C)(C)OC(=O)N(C(=O)OC(C)(C)C)c1cccc(C#N)n1 ; CC(C)(C)OC(=O)N(C(=O)OC(C)(C)C)c1cccc(CN)n1]

02 (500 mg, 1.563 mmol) was dissolved in tetrahydrofuran (10 mL) at room temperature, and Raney nickel (100 mg) was added. The reaction mixture was stirred in a hydrogen atmosphere at room temperature for 16 h and filtered, and the filtrate was concentrated to dryness by rotary evaporation to give the target product as a colorless sticky liquid (400 mg, crude). LC-MS: 324.3 [M+H]⁺.

### Step 3

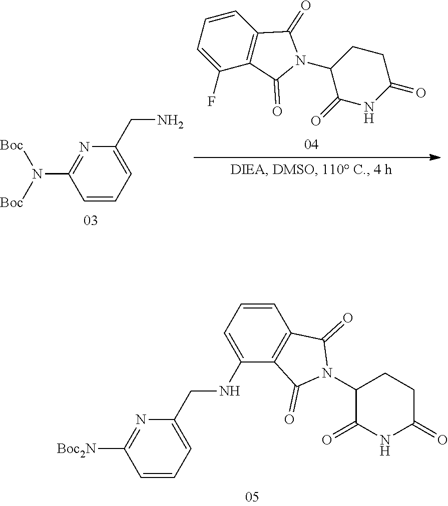

03 (400 mg, 1.238 mmol), 04 (634 mg, 2.477 mmol) and N,N-diisopropylethylamine (320 mg, 2.477 mmol) were dissolved in dimethyl sulfoxide (10 mL) at room temperature. The reaction mixture was stirred at 120° C. for 2 h. Water (100 mL) was added, followed by extraction with ethyl acetate (100 mL×3). The organic phase was washed with saturated brine (100 mL), dried over anhydrous sodium sulfate, and filtered, and the filtrate was concentrated to dryness by rotary evaporation. The residue was purified through a silica gel column (petroleum ether:ethyl acetate=1:1 to 1:2) to give the target product as a yellow solid (400 mg, 55.2% yield). LC-MS: 580.3 [M+H]⁺.

### Step 4

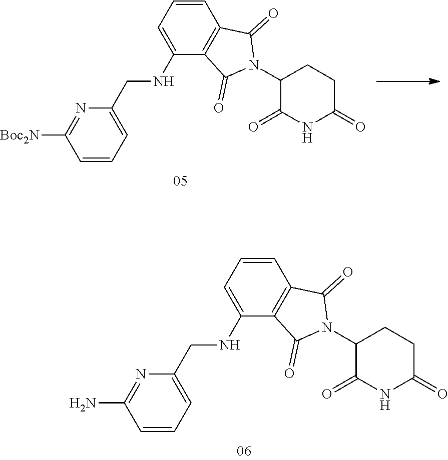

05 (400 mg, 0.690 mmol) was dissolved in dichloromethane (10 mL) at room temperature, and trifluoroacetic acid (3 mL) was added dropwise with stirring at room temperature. The reaction mixture was stirred at room temperature for 6 h and concentrated by rotary evaporation to remove the solvent to give the target product as a yellow oil (230 mg, crude). LC-MS: 380.2 [M+H]⁺.

### Step 5

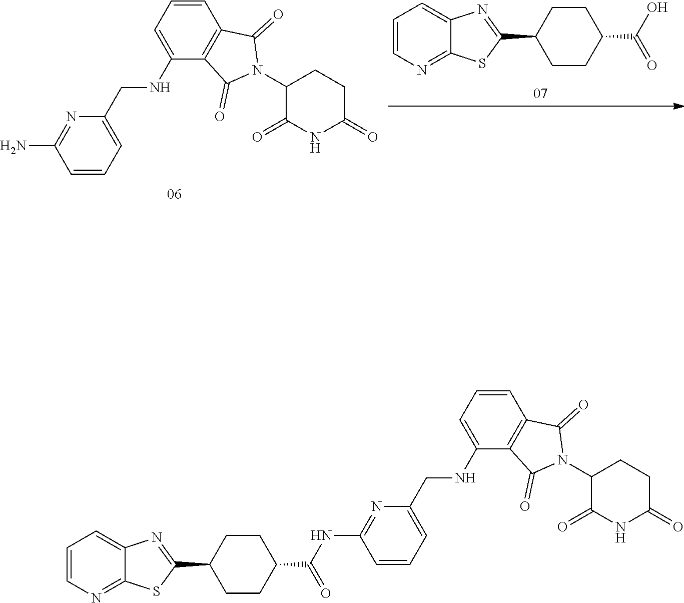 [SMILES: Nc1cccc(CNc2cccc3c2C(=O)N(C2CCC(=O)NC2=O)C3=O)n1 ; O=C(O)[C@H]1CC[C@H](c2nc3cccnc3s2)CC1 ; O=C1CCC(N2C(=O)c3cccc(NCc4cccc(NC(=O)[C@H]5CC[C@H](c6nc7cccnc7s6)CC5)n4)c3C2=O)C(=O)N1]

06 (100 mg, 0.264 mmol), 07 (138 mg, 0.528 mmol), triethylamine (63 mg, 0.634 mmol) and 2-chloro-1-methylpyridinium iodide (81 mg, 0.317 mmol) were dissolved in tetrahydrofuran (2 mL) at room temperature, and the reaction mixture was stirred at 60° C. for 5 days and purified by preparative chromatography (C18, 5-100% acetonitrile/water as mobile phase, 0.1% formic acid) to separate the target product as a yellow solid (2.28 mg, 1.4% yield). LC-MS: 624.4 [M+H]⁺. ¹H NMR (400 MHz, DMSO) δ 11.11 (s, 1H), 10.49 (s, 1H), 8.59 (d, J=3.8 Hz, 1H), 8.35 (d, J=8.0 Hz, 1H), 8.01 (d, J=8.1 Hz, 1H), 7.73 (dd, J=15.2, 7.4 Hz, 1H), 7.60-7.51 (m, 2H), 7.29 (s, 1H), 7.06 (d, J=7.2 Hz, 2H), 6.95 (d, J=8.5 Hz, 1H), 5.09 (dd, J=12.9, 5.3 Hz, 1H), 4.58 (d, J=5.7 Hz, 2H), 3.19 (s, 1H), 2.97-2.84 (m, 1H), 2.73-2.56 (m, 3H), 2.27 (s, 2H), 2.10-1.96 (m, 3H), 1.65 (t, J=10.0 Hz, 4H).

### Example 63

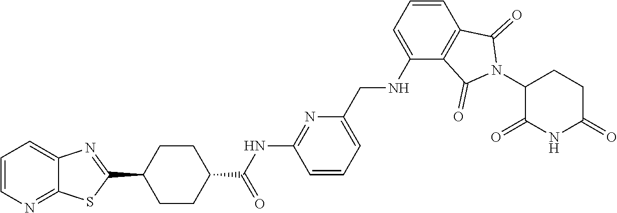 [SMILES: O=C1CCC(N2C(=O)c3cccc(NCc4cccc(NC(=O)[C@H]5CC[C@H](c6nc7cccnc7s6)CC5)n4)c3C2=O)C(=O)N1]

### Preparation Steps

### Step 1

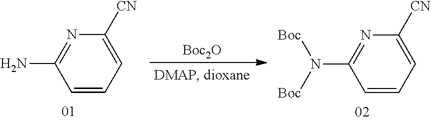 [SMILES: "Boc2O" ; N#Cc1cccc(N)n1 ; CC(C)(C)OC(=O)N(C(=O)OC(C)(C)C)c1cccc(C#N)n1]

01 (500 mg, 4.20 mmol), di-tert-butyl dicarbonate (1373 mg, 6.30 mmol) and 4-dimethylaminopyridine (512 mg, 4.20 mmol) were dissolved in hydrochloric acid in 1,4-dioxane (10 mL), and the reaction mixture was stirred at 50° C. for 2 h. Water (60 mL) was added to the mixture, followed by extraction with ethyl acetate (40 mL×3). The organic phase was washed with saturated brine (60 mL), dried over anhydrous sodium sulfate, and filtered, and the filtrate was concentrated to dryness by rotary evaporation. The residue was purified through a flash silica gel column (petroleum ether:ethyl acetate=5:1) to give the target product as a white solid (800 mg, 59.7% yield). LC-MS: 320.3 [M+H]⁺.

### Step 2

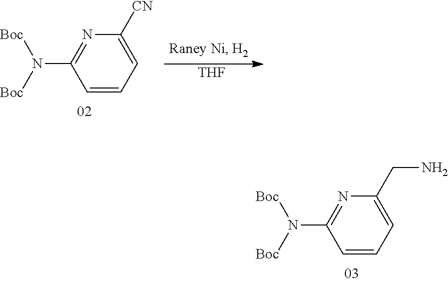 [SMILES: CC(C)(C)OC(=O)N(C(=O)OC(C)(C)C)c1cccc(C#N)n1 ; CC(C)(C)OC(=O)N(C(=O)OC(C)(C)C)c1cccc(CN)n1 ; "THF"]

02 (500 mg, 1.57 mmol) was dissolved in tetrahydrofuran (10 mL) at room temperature, and Raney nickel (300 mg) was added at room temperature. The reaction mixture was purged with hydrogen twice, stirred in a hydrogen atmosphere at room temperature for 16 h, and filtered. The filtrate was concentrated to dryness by rotary evaporation to give the target product as a yellow oil (400 mg, 78.8% yield). LC-MS: 324.4 [M+H]⁺.

### Step 3

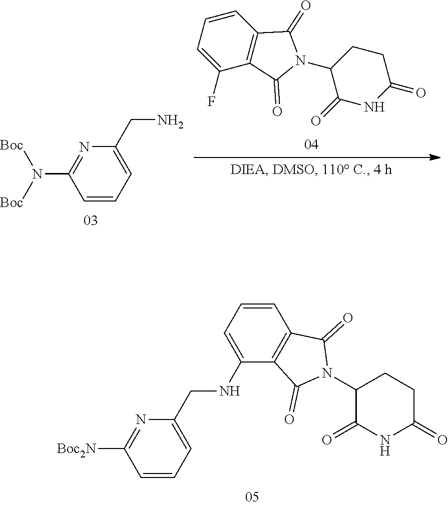

03 (400 mg, 1.24 mmol), 04 (342 mg, 1.24 mmol) and N,N-diisopropylethylamine (480 mg, 3.72 mmol) were dissolved in anhydrous dimethyl sulfoxide (4 mL) at room temperature, and the reaction mixture was stirred at 110° C. for 16 h. Water (80 mL) was added, followed by extraction with ethyl acetate (50 mL×3). The organic phase was washed with saturated brine (50 mL), dried over anhydrous sodium sulfate, and filtered, and the filtrate was concentrated to dryness by rotary evaporation. The residue was purified through a reversed-phase column (C18, water:acetonitrile=100:0 to 35:65, 0.1% formic acid) to give the target product as a yellow solid (349 mg, 48.6% yield). LC-MS: 480.4 [M+H−100]⁺.

### Step 4

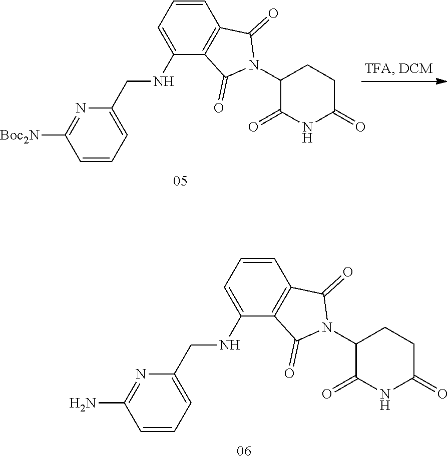

05 (349 mg, 0.603 mmol) was dissolved in dichloromethane (3 mL) at room temperature, and trifluoroacetic acid (1 mL) was added dropwise with stirring at room temperature. The reaction mixture was stirred at room temperature for 4 h and concentrated by rotary evaporation to remove the solvent to give the target product as a yellow oil (100 mg, crude). LC-MS: 380.3 [M+H]⁺.

### Step 5

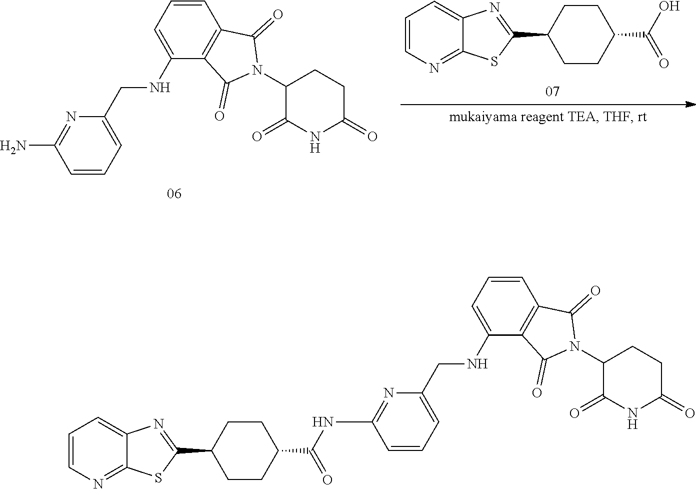 [SMILES: Nc1cccc(CNc2cccc3c2C(=O)N(C2CCC(=O)NC2=O)C3=O)n1 ; O=C(O)[C@H]1CC[C@H](c2nc3cccnc3s2)CC1 ; O=C1CCC(N2C(=O)c3cccc(NCc4cccc(NC(=O)[C@H]5CC[C@H](c6nc7cccnc7s6)CC5)n4)c3C2=O)C(=O)N1]

06 (60 mg, 0.158 mmol), 07 (41.5 mg, 0.158 mmol), triethylamine (38.4 mg, 0.38 mmol) and Mukaiyama's reagent (48.4 mg, 0.190 mmol) were dissolved in tetrahydrofuran (1 mL) at room temperature, and the reaction mixture was stirred at room temperature for 16 h and purified by preparative chromatography (C18, 5-100% acetonitrile/water as mobile phase, 0.1% formic acid) to separate the target product as a yellow solid (2.26 mg, 2.3% yield). LC-MS: 624.3 [M+H]⁺. ¹H NMR (400 MHz, DMSO-d₆) δ 11.12 (s, 1H), 8.53 (d, J=4.2 Hz, 1H), 8.35-8.13 (m, 2H), 7.91 (s, 1H), 7.86-7.74 (m, 2H), 7.51 (dd, J=8.1, 5.0 Hz, 1H), 6.52 (d, J=8.9 Hz, 1H), 6.35 (d, 1H), 6.10 (d, J=9.8 Hz, 2H), 5.25-5.07 (m, 2H), 4.23 (t, J=16.0 Hz, 1H), 2.89 (s, 2H), 2.63 (s, 1H), 2.33-2.21 (m, 2H), 2.12-1.97 (m, 4H), 1.88 (s, 1H), 1.66-1.59 (m, 4H).

### Example 64

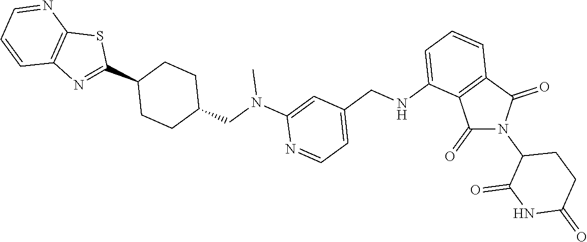 [SMILES: CN(C[C@H]1CC[C@H](c2nc3cccnc3s2)CC1)c1cc(CNc2cccc3c2C(=O)N(C2CCC(=O)NC2=O)C3=O)ccn1]

### Preparation Steps

### Step 1

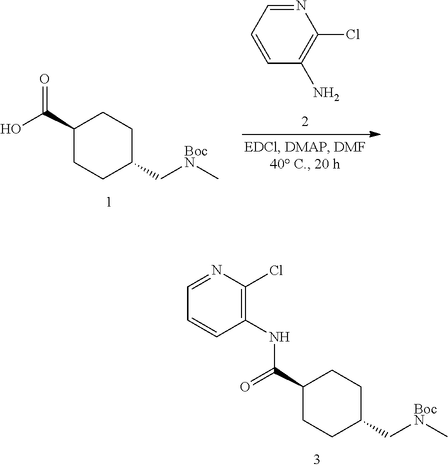 [SMILES: CN(C[C@H]1CC[C@H](C(=O)O)CC1)C(=O)OC(C)(C)C ; Nc1cccnc1Cl ; CN(C[C@H]1CC[C@H](C(=O)Nc2cccnc2Cl)CC1)C(=O)OC(C)(C)C]

1 (1.00 g, 3.69 mmol), 2 (569 mg, 4.43 mmol) and 4-dimethylaminopyridine (586 mg, 4.80 mmol) were dissolved in anhydrous N,N-dimethylformamide (10 mL) at room temperature, and 1-(3-dimethylaminopropyl)-3-ethylcarbodiimide hydrochloride (920 mg, 4.80 mmol) was added in batches with stirring at room temperature. The reaction mixture was stirred at 40° C. for 16 h. Water (50 mL) was added, followed by extraction with ethyl acetate (15 mL×3). The organic phase was washed with saturated brine (50 mL), dried over anhydrous sodium sulfate, and filtered, and the filtrate was concentrated to dryness by rotary evaporation. The residue was purified through a flash silica gel column (ethyl acetate/petroleum ether=0-40%) to give the target product as a yellow oil (780 mg, 55.4% yield). LC-MS: 404.3 [M+Na]⁺; 326.2 [M+H−56]⁺; 282.2 [M+H−100]⁺.

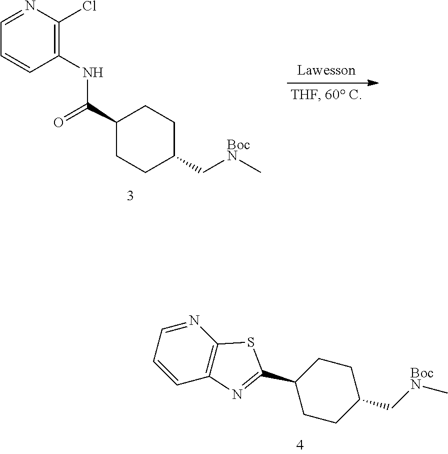 [SMILES: CN(C[C@H]1CC[C@H](C(=O)Nc2cccnc2Cl)CC1)C(=O)OC(C)(C)C ; CN(C[C@H]1CC[C@H](c2nc3cccnc3s2)CC1)C(=O)OC(C)(C)C]

3 (760 mg, 1.99 mmol) and Lawesson's reagent (1.21 g, 2.99 mmol) were mixed in anhydrous tetrahydrofuran (8 mL) at room temperature, and the reaction mixture was purged with nitrogen three times, then stirred at 60° C. for 4 h, cooled to room temperature, and then concentrated to dryness by rotary evaporation. The residue was purified through a flash silica gel column (ethyl acetate/petroleum ether=0-40%) and a reversed-phase column (C18, 10-100% acetonitrile/water as mobile phase, 0.1% formic acid) to give the target product as a colorless oil (575 mg, 79.9% yield). LC-MS: 362.5 [M+H]⁺; 306.4 [M+H−56]⁺; 262.4 [M+H−100]⁺.

### Step 3

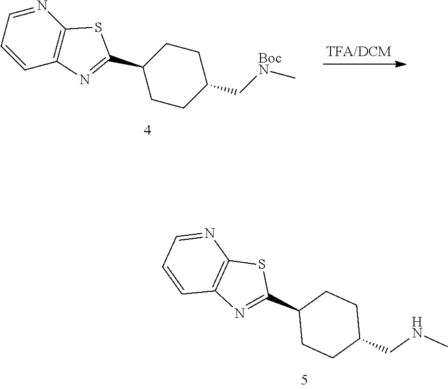 [SMILES: CN(C[C@H]1CC[C@H](c2nc3cccnc3s2)CC1)C(=O)OC(C)(C)C ; CNC[C@H]1CC[C@H](c2nc3cccnc3s2)CC1]

4 (565 mg, 1.56 mmol) was dissolved in dichloromethane (2 mL) at room temperature, and trifluoroacetic acid (2 mL) was added with stirring at room temperature. The reaction mixture was stirred at room temperature for 2 h and concentrated under reduced pressure to remove the solvent to give the target product as a yellow oil (430 mg, crude). LC-MS: 262.4 [M+H]⁺.

### Step 4

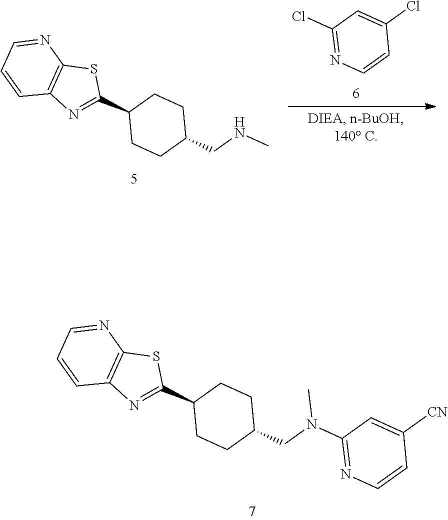 [SMILES: CNC[C@H]1CC[C@H](c2nc3cccnc3s2)CC1 ; Clc1ccnc(Cl)c1 ; CN(C[C@H]1CC[C@H](c2nc3cccnc3s2)CC1)c1cc(C#N)ccn1]

5 (370 mg, 1.42 mmol), 6 (197 mg, 1.42 mmol) and N,N-diisopropylethylamine (918 mg, 7.10 mmol) were dissolved in n-butanol (10 mL) at room temperature, and the reaction mixture was stirred at 140° C. for 16 h, cooled to room temperature, and then concentrated under reduced pressure to remove the solvent. The residue was purified through a flash silica gel column (ethyl acetate:petroleum ether=0-40%) to give the target product as a yellow solid (130 mg, 25.3% yield). LC-MS: 364.4 [M+H]⁺.

### Step 5

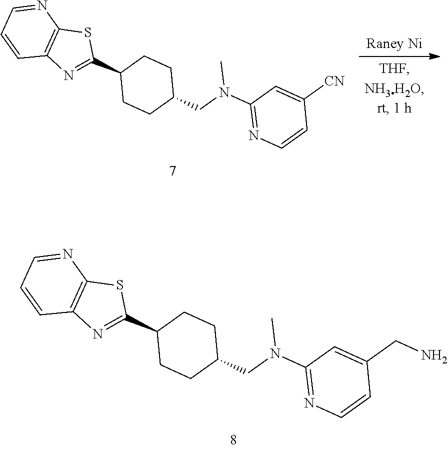 [SMILES: CN(C[C@H]1CC[C@H](c2nc3cccnc3s2)CC1)c1cc(C#N)ccn1 ; CN(C[C@H]1CC[C@H](c2nc3cccnc3s2)CC1)c1cc(CN)ccn1]

7 (110 mg, 0.303 mmol) was dissolved in tetrahydrofuran (5 mL) at room temperature, and a drop of ammonia solution (0.05 mL) and Raney nickel (100 mg) were added successively. The reaction mixture was purged with hydrogen three times, then stirred in a hydrogen atmosphere at room temperature for 1 h, and filtered. The filtrate was concentrated to dryness by rotary evaporation to give the target product as a white solid (40 mg, 36.0% yield). LC-MS: 368.1 [M+H]⁺.

### Step 6

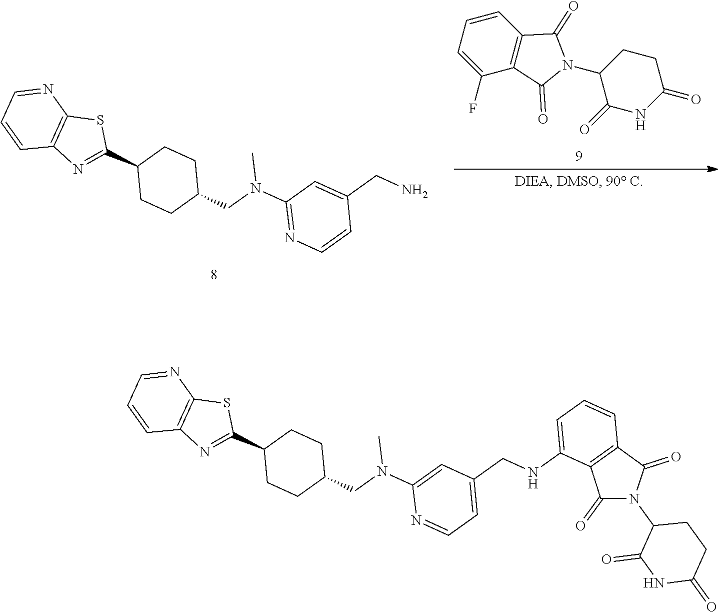 [SMILES: CN(C[C@H]1CC[C@H](c2nc3cccnc3s2)CC1)c1cc(CN)ccn1 ; O=C1CCC(N2C(=O)c3cccc(F)c3C2=O)C(=O)N1 ; CN(C[C@H]1CC[C@H](c2nc3cccnc3s2)CC1)c1cc(CNc2cccc3c2C(=O)N(C2CCC(=O)NC2=O)C3=O)ccn1]

8 (30.0 mg, 0.082 mmol), 9 (18.1 mg, 0.066 mmol) and N,N-diisopropylethylamine (31.8 mg, 0.246 mmol) were dissolved in anhydrous dimethyl sulfoxide (0.5 mL) at room temperature, and the reaction mixture was stirred at 90° C. for 16 h and purified by preparative reversed-phase liquid chromatography (C18, 5-100% acetonitrile/water as mobile phase, 0.1% formic acid) and preparative thin-layer chromatography (dichloromethane/methanol=20:1) to separate the target product as a yellow solid (2.51 mg, 6.1% yield). LC-MS: 624.2 [M+H]⁺, ¹H NMR (400 MHz, DMSO) δ 11.09 (s, 1H), 8.56 (dd, J=4.6, 1.5 Hz, 1H), 8.32 (dd, J=8.2, 1.5 Hz, 1H), 7.99 (d, J=5.1 Hz, 1H), 7.60-7.47 (m, 2H), 7.25 (t, J=6.3 Hz, 1H), 7.04 (d, J=7.0 Hz, 1H), 6.92 (d, J=8.6 Hz, 1H), 6.53 (s, 1H), 6.50 (d, J=5.2 Hz, 1H), 5.07 (dd, J=12.5, 5.4 Hz, 1H), 4.50 (d, J=6.3 Hz, 2H), 3.55-3.52 (m, 1H), 3.07-2.98 (m, 4H), 2.92-2.83 (m, 1H), 2.59-2.54 (m, 2H), 2.16-2.07 (m, 2H), 2.07-1.95 (m, 2H), 1.75-1.63 (m, 3H), 1.50-1.40 (m, 2H), 1.17-1.05 (m, 2H).

### Example 65

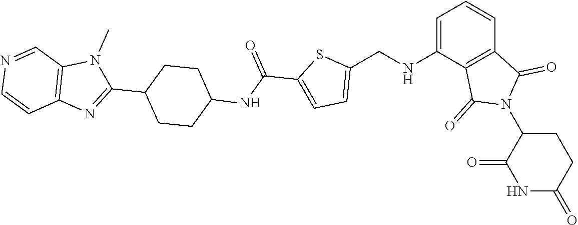 [SMILES: Cn1c(C2CCC(NC(=O)c3ccc(CNc4cccc5c4C(=O)N(C4CCC(=O)NC4=O)C5=O)s3)CC2)nc2ccncc21]

### Preparation Steps

### Step 1

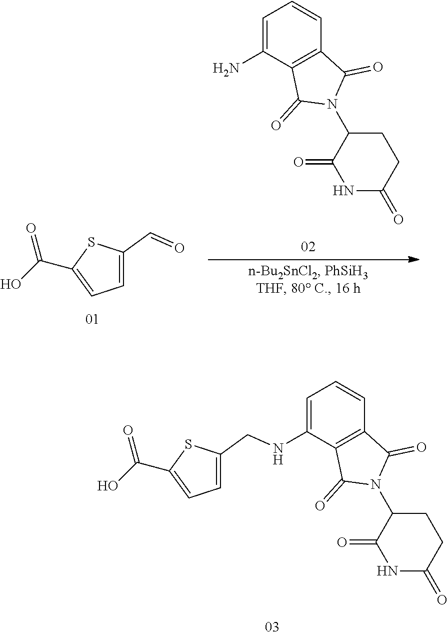 [SMILES: Nc1cccc2c1C(=O)N(C1CCC(=O)NC1=O)C2=O ; O=Cc1ccc(C(=O)O)s1 ; O=C1CCC(N2C(=O)c3cccc(NCc4ccc(C(=O)O)s4)c3C2=O)C(=O)N1]

01 (80 mg, 0.51 mmol) and 02 (118 mg, 0.43 mmol) were dissolved in THF (2 mL) at room temperature, and dibutyltin chloride (155 mg, 0.51 mmol) and triphenylsilane (47 mg, 0.43 mmol) were added. The mixture was reacted in a sealed tube at 80° C. for 16 h and cooled to room temperature. The reaction mixture was diluted with water (50 mL) and extracted with ethyl acetate (50 mL×3), and the organic phases were washed with saturated brine (25 mL×3), combined, dried over anhydrous sodium sulfate, and concentrated to give a crude product, which was then purified by column chromatography (petroleum ether:ethyl acetate=1:2) to give a green solid (50 mg, 23.6% yield). LC-MS: 414.4 [M+H]⁺.

### Step 2

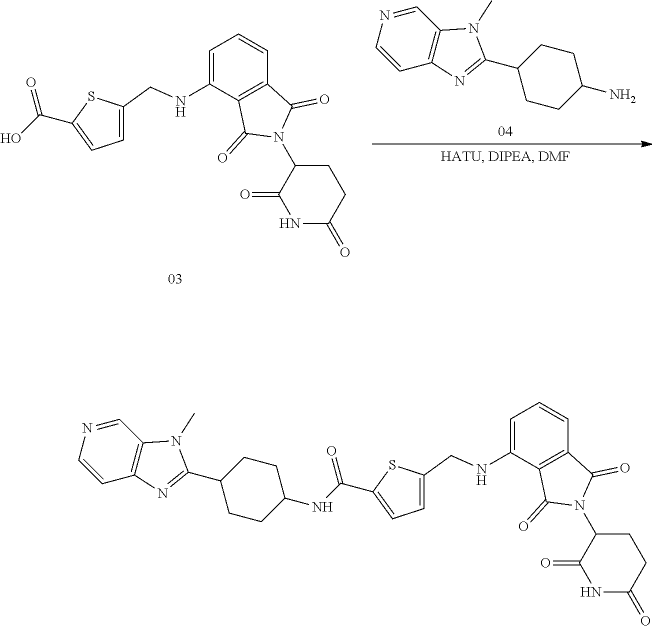 [SMILES: O=C1CCC(N2C(=O)c3cccc(NCc4ccc(C(=O)O)s4)c3C2=O)C(=O)N1 ; Cn1c(C2CCC(N)CC2)nc2ccncc21 ; Cn1c(C2CCC(NC(=O)c3ccc(CNc4cccc5c4C(=O)N(C4CCC(=O)NC4=O)C5=O)s3)CC2)nc2ccncc21]

03 (25 mg, 0.06 mmol) and 04 (16 mg, 0.06 mmol) were dissolved in DMF (5 mL) at room temperature, and HATU (34 mg, 0.09 mmol) and DIEA (23 mg, 0.18 mmol) were added. The mixture was stirred at room temperature for 16 h. The reaction mixture was diluted with water (100 mL) and extracted with ethyl acetate (75 mL×3). The organic phases were washed with saturated brine (30 mL×3), combined, dried over anhydrous sodium sulfate, and then concentrated, and the residue was purified by preparative chromatography to give PRJ2-3098 as a yellow solid (2.22 mg, 5.9% yield). LC-MS: 626.7 [M+H]⁺. ¹HNMR (400 MHz, DMSO-d₆) δ 11.10 (s, 1H), 8.87 (s, 1H), 8.42 (s, 1H), 8.31-8.24 (m, 2H), 7.66 (d, J=3.7 Hz, 1H), 7.60-7.54 (m, 2H), 7.32 (t, J=6.1 Hz, 1H), 7.08 (dd, J=7.7, 5.0 Hz, 3H), 5.08 (dd, J=12.8, 5.3 Hz, 1H), 4.76 (d, J=6.1 Hz, 2H), 3.90 (d, J=8.4 Hz, 3H), 3.78 (d, J=7.8 Hz, 1H), 3.05-2.96 (m, 2H), 2.92-2.85 (m, 1H), 2.07-1.96 (m, 6H), 1.75-1.72 (m, 2H), 1.56-1.53 (m, 2H).

### Example 66

### Preparation Steps

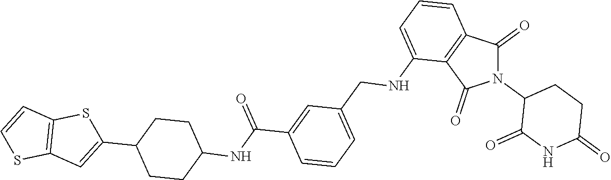 [SMILES: O=C1CCC(N2C(=O)c3cccc(NCc4cccc(C(=O)NC5CCC(c6cc7sccc7s6)CC5)c4)c3C2=O)C(=O)N1]

### Step 1

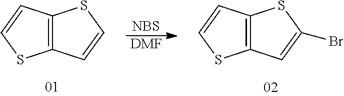

01 (500 mg, 3.57 mmol) was dissolved in anhydrous N,N-dimethylformamide (10 mL), and N-bromosuccinimide (635 mg, 3.57 mmol) was added at 0° C. The reaction mixture was stirred at 0° C. for 16 h. Water (100 mL) was added, followed by extraction with ethyl acetate (20 mL×2). The organic phase was washed with saturated brine (20 mL), dried over anhydrous sodium sulfate, and filtered, and the filtrate was concentrated to dryness by rotary evaporation. The residue was purified through a reversed-phase column (C18, 10-100% acetonitrile/water as mobile phase, 0.03% trifluoroacetic acid) to give the target product as a brown oil (463 mg, 59.3% yield).

### Step 2

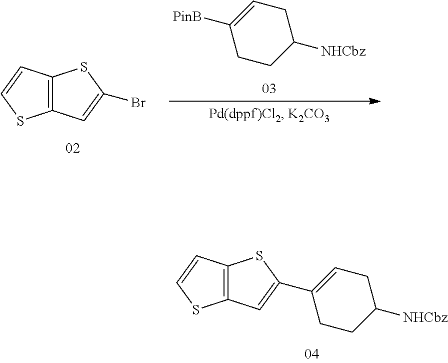

02 (263 mg, 1.20 mmol), 03 (429 mg, 1.20 mmol), potassium carbonate (498 mg, 3.60 mmol) and [1,1′-bis(diphenylphosphino)ferrocene]palladium (II) dichloride (87.8 mg, 0.120 mmol) were mixed in dioxane (10 mL) and water (2 mL), and the reaction mixture was purged with argon, stirred in an argon atmosphere at 90° C. for 16 h, cooled to room temperature, and then concentrated under reduced pressure to remove the solvent. Water (100 mL) was added, followed by extraction with ethyl acetate (30 mL×3). The organic phase was washed with saturated brine (60 mL), dried over anhydrous sodium sulfate, and filtered, and the filtrate was concentrated to dryness by rotary evaporation. The residue was purified through a flash silica gel column (petroleum ether:ethyl acetate=5:1) to give the target product as a white solid (167 mg, 37.7% yield). LC-MS: 370.2 [M+H]⁺.

### Step 3

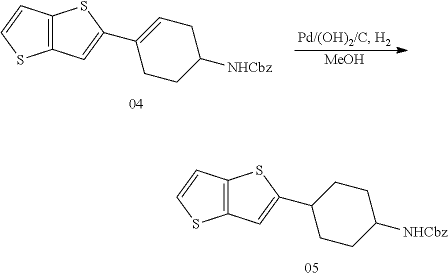 [SMILES: "MeOH" ; O=C(NC1CC=C(c2cc3sccc3s2)CC1)OCc1ccccc1 ; O=C(NC1CCC(c2cc3sccc3s2)CC1)OCc1ccccc1]

04 (167 mg, 0.452 mmol) was dissolved in tetrahydrofuran (5 mL), and then 20% palladium hydroxide/carbon (100 mg) was added. The reaction mixture was stirred at room temperature for 16 h and filtered, and the filtrate was concentrated to dryness by rotary evaporation. The residue was purified through a reversed-phase column (C18, 10%-100% acetonitrile/water as the mobile phase, 0.03% trifluoroacetic acid) to give the target product as a white solid (106 mg, 63.1% yield). LC-MS: 372.3 [M+H]⁺.

### Step 4

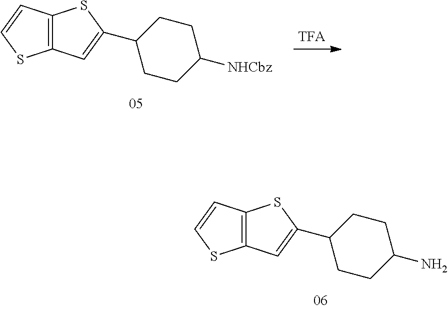 [SMILES: O=C(NC1CCC(c2cc3sccc3s2)CC1)OCc1ccccc1 ; NC1CCC(c2cc3sccc3s2)CC1]

05 (106 mg, 0.285 mmol) was dissolved in trifluoroacetic acid (2 mL), and the reaction mixture was stirred at 70° C. for 30 min and concentrated to dryness by rotary evaporation. The residue was purified through a reversed-phase column (C18, 10%-100% acetonitrile/water as mobile phase, 0.03% trifluoroacetic acid) to give the target product as a green solid (6.00 mg, 3.62% yield). LC-MS: 238.0 [M+H]⁺.

### Step 5

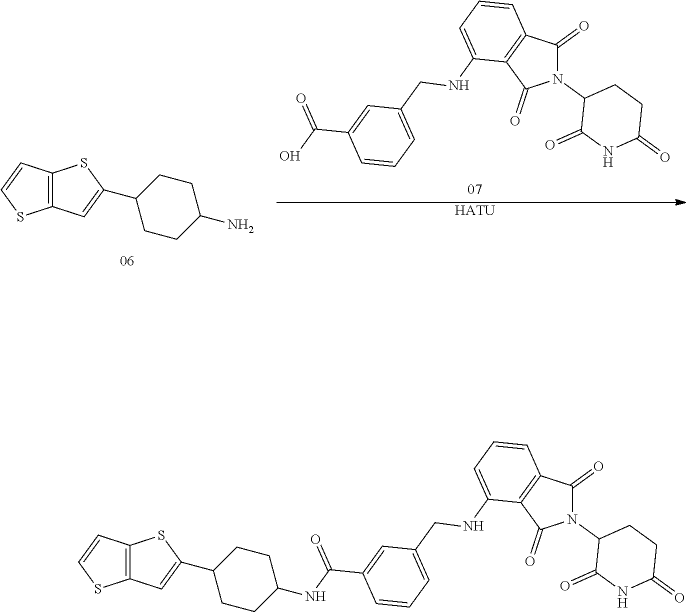 [SMILES: NC1CCC(c2cc3sccc3s2)CC1 ; O=C1CCC(N2C(=O)c3cccc(NCc4cccc(C(=O)O)c4)c3C2=O)C(=O)N1 ; "HATU" ; O=C1CCC(N2C(=O)c3cccc(NCc4cccc(C(=O)NC5CCC(c6cc7sccc7s6)CC5)c4)c3C2=O)C(=O)N1]

06 (6.00 mg, 0.025 mmol), 07 (10.2 mg, 0.025 mmol) and N,N-diisopropylethylamine (9.69 mg, 0.075 mmol) were mixed in anhydrous N,N-dimethylformamide (1 mL) at room temperature, and (7-azabenzotriazole)-tetramethyluronium hexafluorophosphate (14.4 mg, 0.038 mmol) was added in batches with stirring at room temperature. The reaction mixture was stirred at room temperature for 2 h. The mixture was purified by preparative chromatography (C18, 5-100% acetonitrile/water as mobile phase, 0.1% formic acid) to separate the target product as a yellow solid (13.92 mg, 87.9% yield). LC-MS: 627.1 [M+H]⁺.

¹H NMR (400 MHz, DMSO-d₆) δ 11.10 (s, 1H), 8.30-8.13 (m, 1H), 7.88-7.82 (m, 1H), 7.75-7.68 (m, 1H), 7.56-7.53 (m, 1H), 7.52-7.45 (m, 2H), 7.43-7.34 (m, 2H), 7.32-7.22 (m, 2H), 7.02 (t, J=6.8 Hz, 1H), 6.97-6.92 (m, 1H), 5.10-5.04 (m, 1H), 4.64-4.56 (m, 2H), 4.02-3.85 (m, 1H), 3.15-3.07 (m, 1H), 2.91-2.83 (m, 1H), 2.64-2.53 (m, 2H), 2.13-2.01 (m, 3H), 1.98-1.93 (m, 1H), 1.92-1.84 (m, 1H), 1.83-1.74 (m, 1H), 1.73-1.66 (m, 1H), 1.63-1.47 (m, 2H).

### Example 67

### Synthesis of

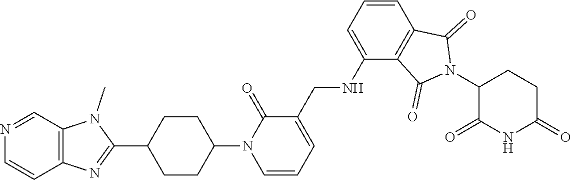 [SMILES: Cn1c(C2CCC(n3cccc(CNc4cccc5c4C(=O)N(C4CCC(=O)NC4=O)C5=O)c3=O)CC2)nc2ccncc21]

### Example 68

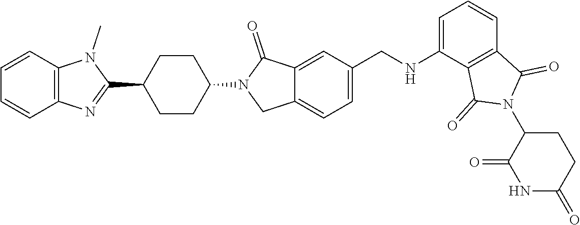 [SMILES: Cn1c2ccccc2nc1[C@H]1CC[C@H](N2Cc3ccc(CNc4cccc5c4C(=O)N(C4CCC(=O)NC4=O)C5=O)cc3C2=O)CC1]

### Preparation Steps

### Step 1

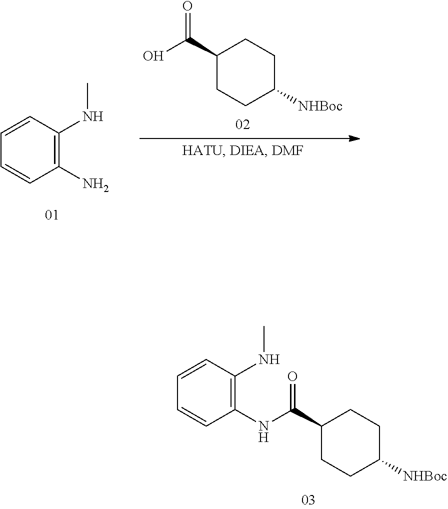

01 (2.51 g, 20.53 mmol), 02 (5.0 g, 20.53 mmol) and N,N-diisopropylethylamine (7.97 g, 61.59 mmol) were dissolved in N,N-dimethylformamide (50 mL) at room temperature, and (7-azabenzotriazole)-tetramethyluronium hexafluorophosphate (9.38 g, 24.64 mmol) was added with stirring at room temperature. The reaction mixture was stirred at room temperature for 2 h. Water (100 mL) was added, and a solid precipitated. The mixture was filtered, and the resulting filter cake was dried by rotary evaporation to give the target product as a white solid (5.7 g, 80% yield). LC-MS: 348.2 [M+H]⁺.

### Step 2

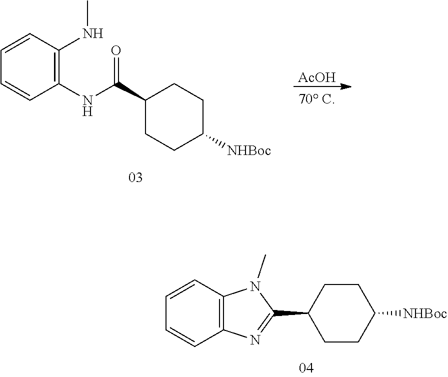 [SMILES: "AcOH" ; CNc1ccccc1NC(=O)[C@H]1CC[C@H](NC(=O)OC(C)(C)C)CC1 ; Cn1c2ccccc2nc1[C@H]1CC[C@H](NC(=O)OC(C)(C)C)CC1]

03 (5.7 g, 16.43 mmol) was dissolved in acetic acid (30 mL) at room temperature, and the reaction mixture was stirred in an argon atmosphere at 70° C. for 2 h and concentrated by rotary evaporation to remove the solvent to give the target product as a yellow oil (5.1 g, 94.3% yield). LC-MS: 330.2 [M+H]⁺.

### Step 3

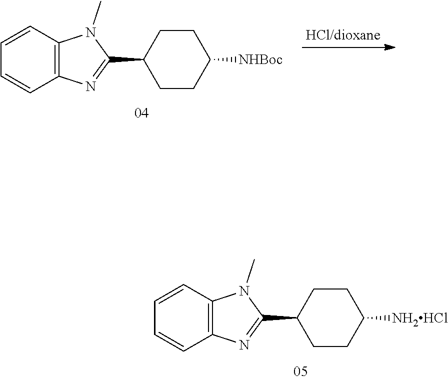

04 (1 g, 3.04 mmol) was dissolved in dichloromethane (10 mL) at room temperature, and hydrochloric acid in 1,4-dioxane (10 mL) was added. The reaction mixture was stirred at room temperature for 2 h and concentrated to dryness by rotary evaporation to give the target product as a white solid (800 mg, 99.3% yield). LC-MS: 230.1 [M+H]⁺.

### Step 4

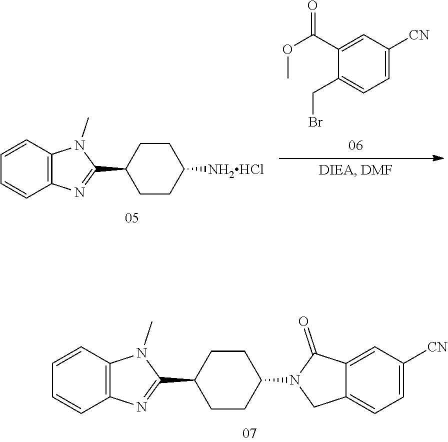

05 (100 mg, 0.37 mmol), 06 (96 mg, 0.37 mmol) and N,N-diisopropylethylamine (243 mg, 1.89 mmol) were dissolved in N,N-dimethylformamide (2 mL) at room temperature, and the reaction mixture was stirred at 90° C. for 2 h. Water (20 mL) was added, followed by extraction with ethyl acetate (50 mL×3). The organic phase was washed with saturated brine (50 mL), dried over anhydrous sodium sulfate, and filtered, and the filtrate was concentrated to dryness by rotary evaporation. The residue was passed through a silica gel column (dichloromethane:methanol=10:1) to separate the target product as a white solid (50 mg, 36.5% yield). LC-MS: 371.2 [M+H]⁺.

### Step 5

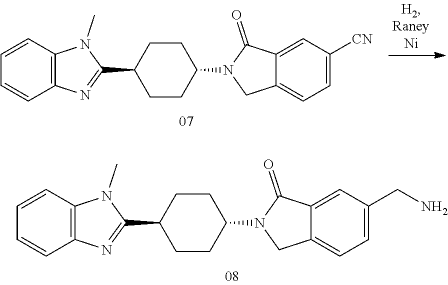 [SMILES: Cn1c2ccccc2nc1[C@H]1CC[C@H](N2Cc3ccc(C#N)cc3C2=O)CC1 ; Cn1c2ccccc2nc1[C@H]1CC[C@H](N2Cc3ccc(CN)cc3C2=O)CC1]

07 (50 mg, 0.135 mmol) was dissolved in tetrahydrofuran (2 mL) at room temperature, and Raney nickel (20 mg) was added at room temperature. The reaction mixture was purged with hydrogen twice, stirred in a hydrogen atmosphere at room temperature for 6 h, and filtered, and the filtrate was concentrated to dryness by rotary evaporation to give the target product as a colorless sticky liquid (42 mg, 83.2% yield). LC-MS: 375.4 [M+H]⁺.

### Step 6

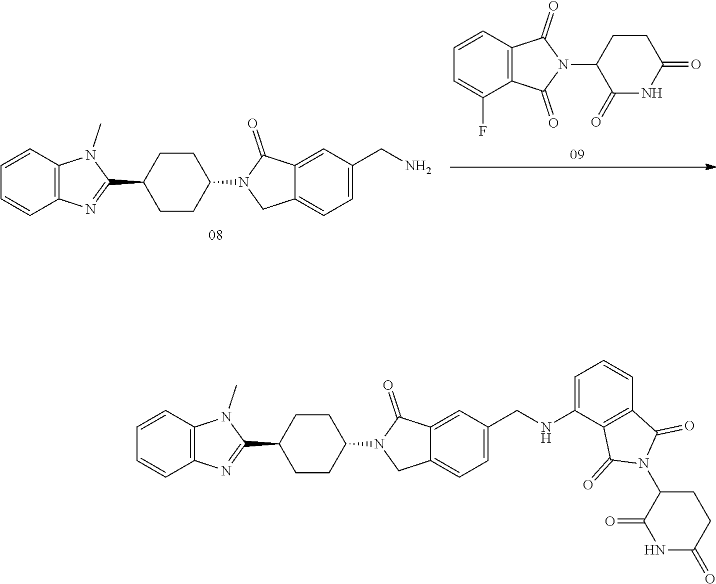 [SMILES: Cn1c2ccccc2nc1[C@H]1CC[C@H](N2Cc3ccc(CN)cc3C2=O)CC1 ; O=C1CCC(N2C(=O)c3cccc(F)c3C2=O)C(=O)N1 ; Cn1c2ccccc2nc1[C@H]1CC[C@H](N2Cc3ccc(CNc4cccc5c4C(=O)N(C4CCC(=O)NC4=O)C5=O)cc3C2=O)CC1]

08 (42 mg, 0.112 mmol), 09 (22 mg, 0.079 mmol) and N,N-diisopropylethylamine (44 mg, 0.337 mmol) were dissolved in dimethyl sulfoxide (1 mL) at room temperature. The reaction mixture was stirred at 110° C. for 16 h. The mixture was cooled to 20° C. and then purified by preparative chromatography (C18, 5-100% acetonitrile/water as mobile phase, 0.1% formic acid) to separate the target product as a yellow solid (15.24 mg, 21.6% yield). LC-MS: 631.3 [M+H]⁺. ¹H NMR (400 MHz, DMSO-d₆) δ 10.96 (s, 1H), 8.58 (d, J=4.1 Hz, 1H), 8.32 (t, J=16.3 Hz, 1H), 8.11 (dd, J=55.5, 7.4 Hz, 1H), 7.80 (d, J=6.7 Hz, 1H), 7.64-7.42 (m, 3H), 7.33 (s, 2H), 7.11 (dd, J=39.0, 8.4 Hz, 1H), 5.37-5.00 (m, 3H), 4.41-4.11 (m, 2H), 3.88 (dd, J=14.0, 6.1 Hz, 3H), 3.15 (s, 1H), 2.90 (s, 1H), 2.56 (d, J=21.0 Hz, 1H), 2.46-2.35 (m, 1H), 2.12 (dd, J=90.0, 15.3 Hz, 6H), 1.87-1.68 (m, 3H), 1.52 (d, J=13.6 Hz, 1H).

### Example 69

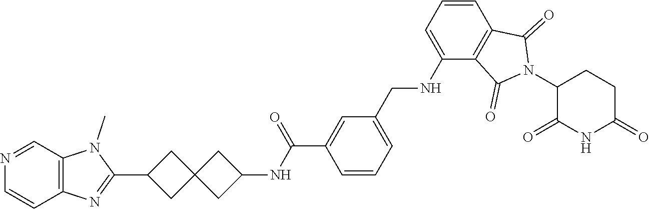 [SMILES: Cn1c(C2CC3(CC(NC(=O)c4cccc(CNc5cccc6c5C(=O)N(C5CCC(=O)NC5=O)C6=O)c4)C3)C2)nc2ccncc21]

### Preparation Steps

### Step 1

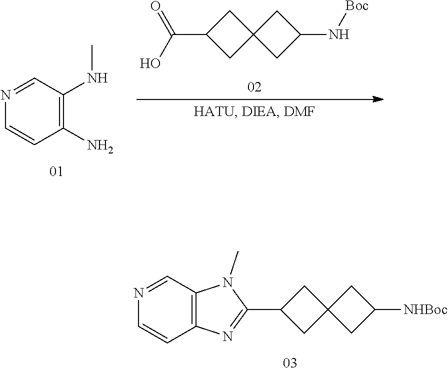

01 (200 mg, 1.62 mmol), 02 (393 mg, 1.54 mmol) and N,N-diisopropylethylamine (628 mg, 4.86 mmol) were dissolved in anhydrous N,N-dimethylformamide (6 mL), and (7-azabenzotriazole)-tetramethyluronium hexafluorophosphate (924 mg, 2.43 mmol) was added in batches with stirring at room temperature. The reaction mixture was stirred at room temperature for 2 h. The mixture was passed through a reversed-phase column (C18, 5-100% acetonitrile/water as mobile phase, 0.1% trifluoroacetic acid) to separate the target product as a yellow solid (26.63 mg, 28.8% yield). LC-MS: 335.1 [M+H]⁺.

### Step 2

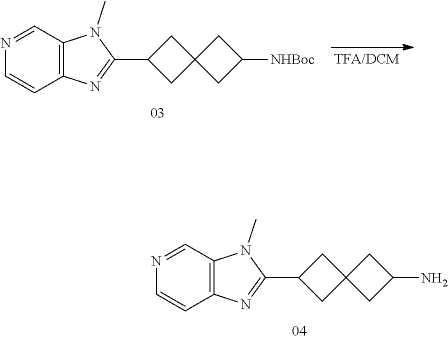 [SMILES: Cn1c(C2CC3(CC(NC(=O)OC(C)(C)C)C3)C2)nc2ccncc21 ; Cn1c(C2CC3(CC(N)C3)C2)nc2ccncc21]

03 (131 mg, 0.383 mmol) was dissolved in dichloromethane (2 mL), and trifluoroacetic acid (0.6 mL) was added dropwise with stirring at room temperature. The reaction mixture was stirred at room temperature for 16 h. The mixture was concentrated under reduced pressure to remove the solvent to give the target product as a brown oil (92 mg, 99.2% yield). LC-MS: 243.1 [M+H]⁺.

### Step 3

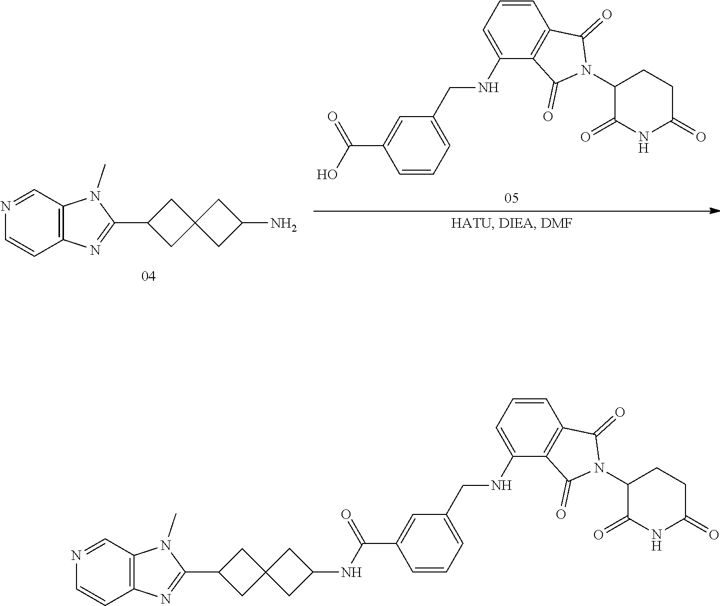 [SMILES: Cn1c(C2CC3(CC(N)C3)C2)nc2ccncc21 ; O=C1CCC(N2C(=O)c3cccc(NCc4cccc(C(=O)O)c4)c3C2=O)C(=O)N1 ; Cn1c(C2CC3(CC(NC(=O)c4cccc(CNc5cccc6c5C(=O)N(C5CCC(=O)NC5=O)C6=O)c4)C3)C2)nc2ccncc21]

04 (36.0 mg, 0.148 mmol), 05 (50.0 mg, 0.123 mmol) and N,N-diisopropylethylamine (48.0 mg, 0.369 mmol) were dissolved in anhydrous N,N-dimethylformamide (1 mL), and (7-azabenzotriazole)-tetramethyluronium hexafluorophosphate (70.0 mg, 0.185 mmol) was added in batches with stirring at room temperature. The reaction mixture was stirred at room temperature for 2 h. The mixture was purified by preparative reversed-phase liquid chromatography (C18, 5-100% acetonitrile/water as mobile phase, 0.1% trifluoroacetic acid) to separate the target product as a yellowish-green solid (30.7 mg, 32.7% yield). LC-MS: 632.4 [M+H]⁺, ¹H NMR (400 MHz, DMSO-d₆) δ 11.07 (s, 1H), 8.82 (s, 1H), 8.55 (d, J=7.5 Hz, 1H), 8.25 (d, J=5.5 Hz, 1H), 7.83 (s, 1H), 7.70 (d, J=7.6 Hz, 1H), 7.53 (d, J=5.6 Hz, 1H), 7.51-7.46 (m, 2H), 7.39 (t, J=7.6 Hz, 1H), 7.26 (t, J=6.2 Hz, 1H), 7.00 (d, J=7.0 Hz, 1H), 6.92 (d, J=8.5 Hz, 1H), 5.05 (dd, J=12.9, 5.3 Hz, 1H), 4.58 (d, J=6.0 Hz, 2H), 4.34-4.27 (m, 1H), 3.81-3.76 (m, 1H), 3.74 (s, 3H), 2.91-2.82 (m, 1H), 2.61-2.52 (m, 5H), 2.46-2.38 (m, 2H), 2.28-2.17 (m, 2H), 2.10-2.00 (m, 2H).

### Example 70

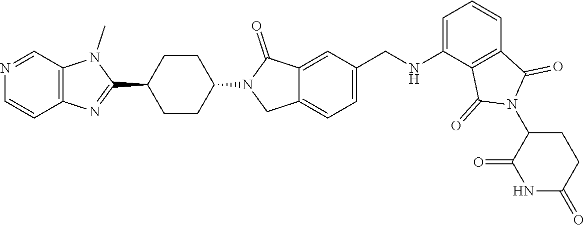 [SMILES: Cn1c2cnccc2nc1[C@H]1CC[C@H](N2Cc3ccc(CNc4cccc5c4C(=O)N(C4CCC(=O)NC4=O)C5=O)cc3C2=O)CC1]

### Preparation Steps

### Step 1

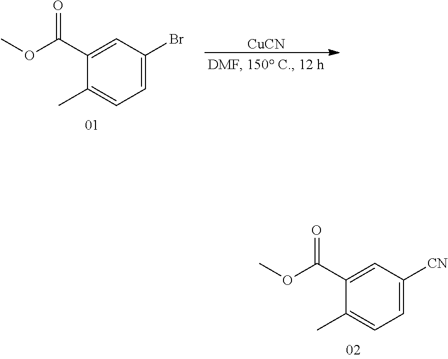

01 (13 g, 56.75 mmol) and cuprous cyanide (7.62 g, 85.08 mmol) were added to a 500 mL single-neck round-bottom flask and dissolved in DMF (100 mL), and the mixture was reacted in a nitrogen atmosphere at 150° C. for 16 h. Water (200 mL) was added, followed by extraction ethyl acetate (200 mL×3). The organic phase was washed with saturated brine (200 mL×2), dried over anhydrous sodium sulfate, and concentrated to dryness by rotary evaporation, and the residue was purified by column chromatography (ethyl acetate/petroleum ether=1:10) to give the target product as a white solid (7.8 g, 79% yield). LC-MS: 176.1 [M+H]⁺.

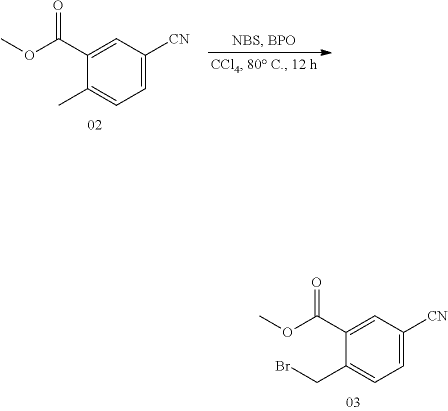

To a 100 mL single-neck round-bottom flask were added 02 (4.1 g, 23.4 mmol), NBS (4.37 g, 24.55 mmol), BPO (569 mg, 2.35 mmol) and CCl₄ (50 mL), and the mixture was reacted in a nitrogen atmosphere at 80° C. for 12 h. The reaction mixture was concentrated to dryness by rotary evaporation, and water (20 mL) was added, followed by extraction with ethyl acetate (20 mL×3). The organic phase was washed with saturated brine (20 mL×2), dried over anhydrous sodium sulfate, and concentrated to dryness by rotary evaporation, and the residue was purified by column chromatography (ethyl acetate/petroleum ether=1:10) to give the target product as a white solid (4.1 g, 69% yield). LC-MS: 253.2/255.2 [M+H]⁺.

Step 3

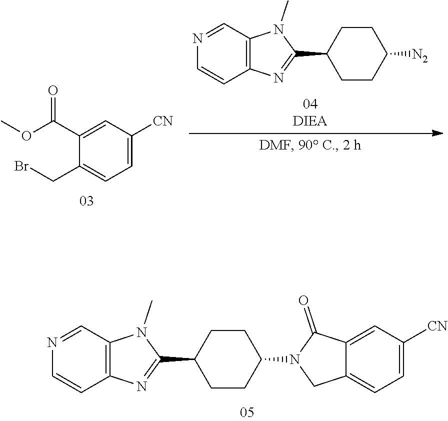

To a 100 mL single-neck round-bottom flask were added 03 (496 mg, 1.95 mmol), 04 (450 mg, 1.95 mmol), DIEA (1.26 g, 9.77 mmol) and DMF (5 mL), and the mixture was reacted in a nitrogen atmosphere at 90° C. for 2 h. Water (20 mL) was added, followed by extraction with ethyl acetate (20 mL×3). The organic phase was washed with saturated brine (20 mL×2), dried over anhydrous sodium sulfate, and concentrated to dryness by rotary evaporation, and the residue was purified by column chromatography (methanol/dichloromethane=1:10) to give the target product as a brown solid (60 mg, 8% yield). LC-MS: 372.2 [M+H]⁺.

### Step 3

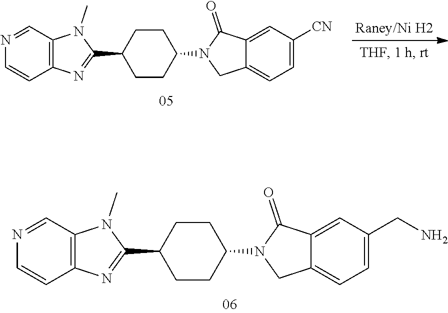 [SMILES: Cn1c2cnccc2nc1[C@H]1CC[C@H](N2Cc3ccc(C#N)cc3C2=O)CC1 ; Cn1c2cnccc2nc1[C@H]1CC[C@H](N2Cc3ccc(CN)cc3C2=O)CC1]

In a 100 mL single-neck flask, 05 (60 mg, 0.162 mmol) was dissolved in tetrahydrofuran (10 mL), and Raney-Ni (60 mg) was added. The reaction mixture was stirred in a hydrogen atmosphere for 2 h and filtered, and the filtrate was concentrated to dryness by rotary evaporation. The residue was purified by column chromatography (methanol/dichloromethane=1:10) to give the target product as a white solid (30 mg, 49% yield). LC-MS: 376.2 [M+H]⁺.

### Step 4

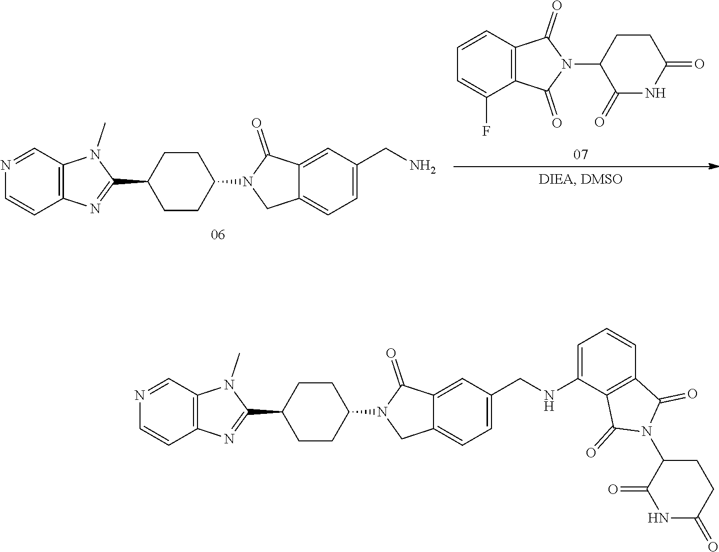 [SMILES: Cn1c2cnccc2nc1[C@H]1CC[C@H](N2Cc3ccc(CN)cc3C2=O)CC1 ; O=C1CCC(N2C(=O)c3cccc(F)c3C2=O)C(=O)N1 ; Cn1c2cnccc2nc1[C@H]1CC[C@H](N2Cc3ccc(CNc4cccc5c4C(=O)N(C4CCC(=O)NC4=O)C5=O)cc3C2=O)CC1]

To a 100 mL single-neck flask were added 06 (30 mg, 0.08 mmol), 07 (22 mg, 0.08 mmol), DIEA (51 mg, 0.4 mmol) and DMSO (1 mL), and the mixture was reacted at room temperature for 2 h and purified by preparative high performance liquid chromatography to give the target product as a yellow solid (9.61 mg, 19% yield). LC-MS: 632.1 [M+H]⁺. ¹H NMR (400 MHz, DMSO-d₆) δ 11.10 (s, 1H), 8.88 (s, 1H), 8.28 (s, 1H), 7.71 (s, 1H), 7.62 (s, 1H), 7.60-7.54 (m, 2H), 7.49 (t, J=7.6 Hz, 1H), 7.40 (s, 1H), 7.02 (d, J=6.8 Hz, 1H), 6.95 (d, J=8.6 Hz, 1H), 5.13-5.01 (m, 1H), 4.66 (s, 2H), 4.48 (s, 2H), 4.12 (s, 1H), 3.90 (s, 3H), 3.09 (s, 1H), 2.94-2.85 (m, 1H), 2.60 (d, J=15.6 Hz, 2H), 2.08 (s, 3H), 2.00 (d, J=8.7 Hz, 1H), 1.89-1.81 (m, 5H).

### Example 71

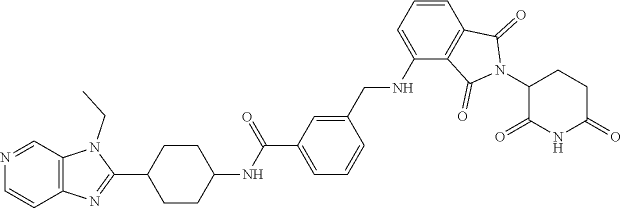 [SMILES: CCn1c(C2CCC(NC(=O)c3cccc(CNc4cccc5c4C(=O)N(C4CCC(=O)NC4=O)C5=O)c3)CC2)nc2ccncc21]

### Preparation Steps

### Step 1

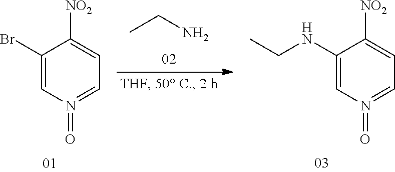

In a 100 mL single-neck flask, 01 (1.5 g, 6.88 mmol) was dissolved in tetrahydrofuran (10 mL), and 02 (17 mL) was added. The mixture was reacted at 50° C. for 2 h, cooled to room temperature, quenched with water (20 mL), and extracted with dichloromethane (20 mL×3), and the organic phase was washed with saturated sodium bicarbonate (20 mL×3) and concentrated to dryness by rotary evaporation to give the target product as a yellow solid (1.3 g, 100% yield). LC-MS: 184.1 [M+H]⁺.

### Step 2

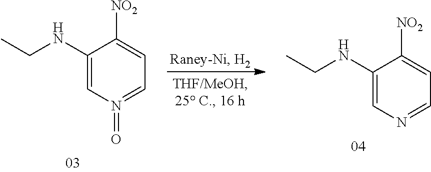

In a 100 mL single-neck flask, 03 (1.3 g, 7.10 mmol) was dissolved in tetrahydrofuran (12 mL) and methanol (30 mL), and Raney nickel (100 mg) was added. The mixture was reacted in a hydrogen atmosphere at room temperature for 16 h and filtered, and the filtrate was concentrated to dryness by rotary evaporation to give the target product as a yellow solid (400 mg, 41.1% yield).

LC-MS: 138.2 [M+H]⁺.

### Step 3

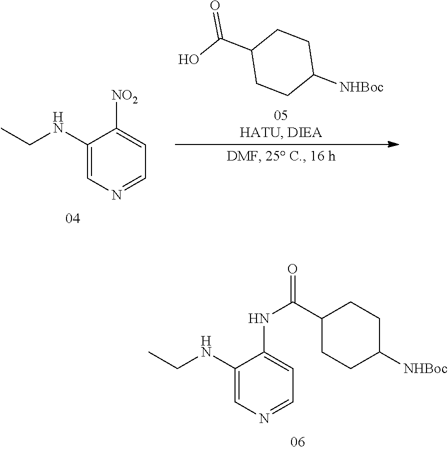 [SMILES: CCNc1cnccc1[N+](=O)[O-] ; CC(C)(C)OC(=O)NC1CCC(C(=O)O)CC1 ; CCNc1cnccc1NC(=O)C1CCC(NC(=O)OC(C)(C)C)CC1]

In a 100 mL single-neck flask, 04 (400 mg, 2.92 mmol) was dissolved in N,N-dimethylformamide (5 mL), and 05 (674 mg, 2.77 mmol), HATU (1.65 g, 4.38 mmol) and N,N-diisopropylethylamine (1.13 g, 8.76 mmol) were added. The mixture was reacted at room temperature for 16 h, quenched with water (15 mL), and extracted with ethyl acetate (20 mL×3). The organic phase was washed with saturated brine (20 mL×3) and concentrated to dryness by rotary evaporation, and the residue was purified by column chromatography (dichloromethane:methanol=10:1) to give the target product as a yellow solid (300 mg, 28.4% yield).

LC-MS: 363.2 [M+H]⁺.

### Step 4

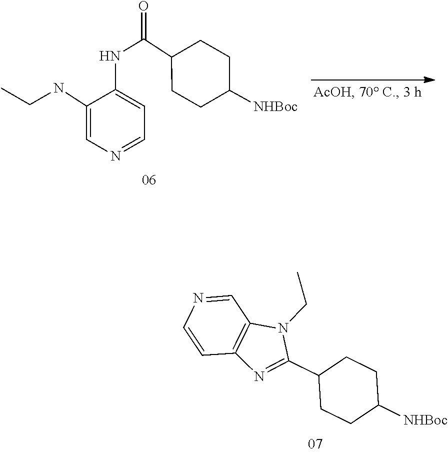

In a 100 mL single-neck flask, 06 (300 mg, 0.829 mmol) was dissolved in acetic acid (2 mL), and the mixture was reacted at 70° C. for 3 h and concentrated to dryness by rotary evaporation. The residue was purified by column chromatography (dichloromethane:methanol=10:1) to give the target product as an orange solid (130 mg, 45.6% yield).

LC-MS: 345.1 [M+H]⁺.

### Step 5

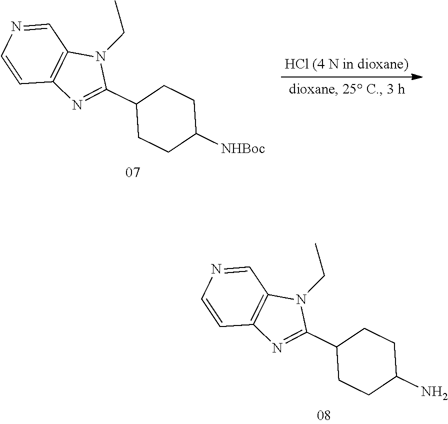 [SMILES: CCn1c(C2CCC(NC(=O)OC(C)(C)C)CC2)nc2ccncc21 ; CCn1c(C2CCC(N)CC2)nc2ccncc21]

In a 50 mL single-neck flask, 07 (130 mg, 0.378 mmol) was dissolved in 1,4-dioxane (5 mL), and hydrochloric acid in 1,4-dioxane (1 mL) was added. The reaction mixture was stirred at room temperature for 3 h and concentrated under reduced pressure to give the target crude product (257 mg), which was directly used in the next step.

LC-MS: 245.2 [M+H]⁺.

### Step 6

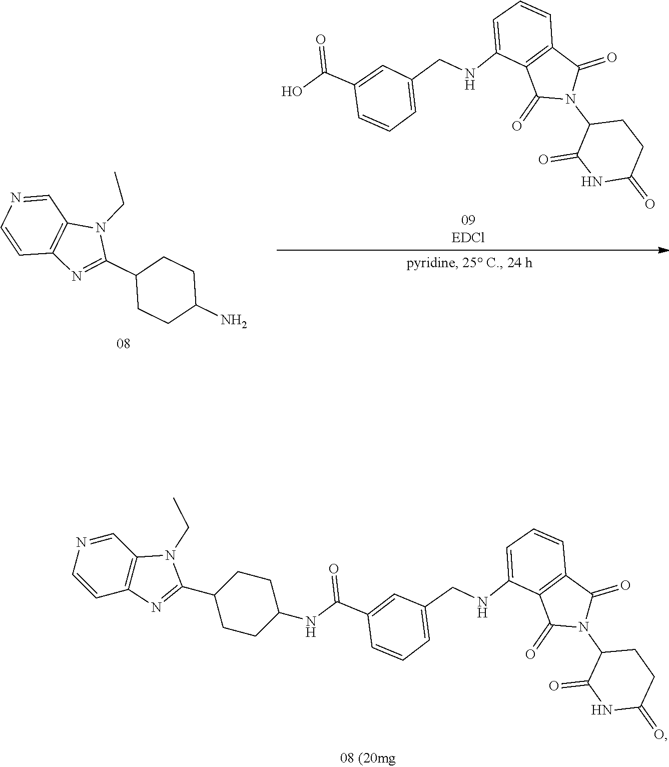 [SMILES: CCn1c(C2CCC(N)CC2)nc2ccncc21 ; O=C1CCC(N2C(=O)c3cccc(NCc4cccc(C(=O)O)c4)c3C2=O)C(=O)N1 ; CCn1c(C2CCC(NC(=O)c3cccc(CNc4cccc5c4C(=O)N(C4CCC(=O)NC4=O)C5=O)c3)CC2)nc2ccncc21]

0.0819 mmol) was dissolved in pyridine (1 mL) at room temperature, and 09 (33 mg, 0.0819 mmol) and EDCI (47 mg, 0.246 mmol) were added successively. The above mixture was stirred at room temperature for 48 h. The reaction mixture was purified by preparative high performance liquid chromatography (FA) to give a yellowish-green solid (4.40 mg, 1.9% yield). LC-MS: 634.3 [M+H]⁺, ¹H NMR (400 MHz, DMSO-d₆) δ 11.10 (s, 1H), 8.91 (s, 1H), 8.39-8.24 (m, 2H), 7.89 (s, 1H), 7.77 (d, J=7.3 Hz, 1H), 7.53 (dd, J=18.5, 6.9 Hz, 3H), 7.41 (t, J=7.5 Hz, 1H), 7.29 (s, 1H), 7.03 (d, J=7.1 Hz, 1H), 6.97 (d, J=8.4 Hz, 1H), 5.09-5.02 (m, 1H), 4.62 (d, J=6.3 Hz, 2H), 4.36 (d, J=7.0 Hz, 2H), 4.07 (s, 1H), 2.97 (d, J=7.0 Hz, 1H), 2.67 (s, 1H), 2.33 (s, 1H), 2.11 (s, 1H), 2.02 (s, 5H), 1.78 (d, J=10.5 Hz, 4H), 1.38 (t, J=7.2 Hz, 3H).

### Example 72

### Synthesis of

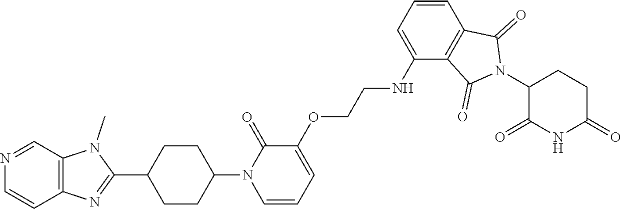 [SMILES: Cn1c(C2CCC(n3cccc(OCCNc4cccc5c4C(=O)N(C4CCC(=O)NC4=O)C5=O)c3=O)CC2)nc2ccncc21]

### Example 73

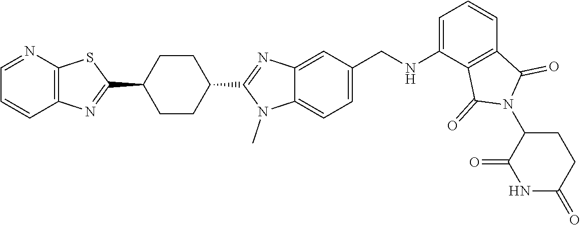 [SMILES: Cn1c2ccc(CNc3cccc4c3C(=O)N(C3CCC(=O)NC3=O)C4=O)cc2nc1[C@H]1CC[C@H](c2nc3cccnc3s2)CC1]

### Preparation Steps

### Step 1

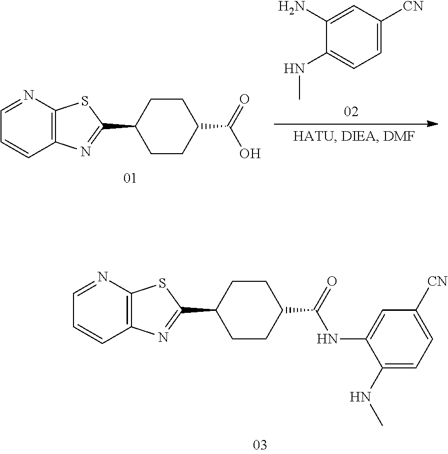

01 (590 mg, 2.25 mmol), 02 (331 mg, 2.25 mmol) and N,N-diisopropylethylamine (872 mg, 6.75 mmol) were dissolved in anhydrous N,N-dimethylformamide (12 mL), and (7-azabenzotriazole)-tetramethyluronium hexafluorophosphate (1.28 g, 3.38 mmol) was added with stirring at room temperature. The reaction mixture was stirred at room temperature for 16 h. Water (20 mL) was added to the mixture, followed by filtration to give the target product as a white solid (500 mg, 56.8% yield). LC-MS: 392.2 [M+H]⁺.

### Step 2

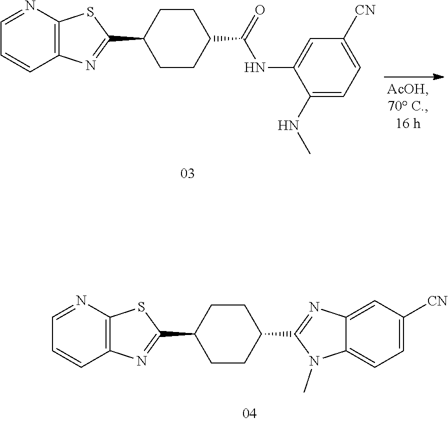

03 (500 mg, 1.28 mmol) was dissolved in acetic acid (10 mL), and the reaction mixture was stirred at 70° C. for 16 h and concentrated to dryness by rotary evaporation to give the target product as a white solid (467 mg, 97.9% yield). LC-MS: 374.3 [M+H]⁺.

### Step 3

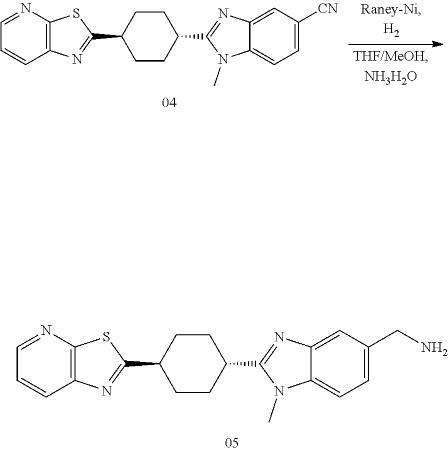 [SMILES: Cn1c2ccc(C#N)cc2nc1[C@H]1CC[C@H](c2nc3cccnc3s2)CC1 ; Cn1c2ccc(CN)cc2nc1[C@H]1CC[C@H](c2nc3cccnc3s2)CC1]

04 (460 mg, 1.23 mmol) was dissolved in tetrahydrofuran (5 mL) and methanol (5 mL), and Raney nickel (200 mg) and 2 drops of ammonia solution were added. The reaction mixture was stirred at room temperature for 8 h and filtered, and the filtrate was concentrated to dryness by rotary evaporation. The residue was purified through a reversed-phase column (C18, 10-100% acetonitrile/water as mobile phase, 0.1% formic acid) to give the target product as a white solid (110 mg, 23.6% yield). LC-MS: 378.5 [M+H]⁺.

### Step 4

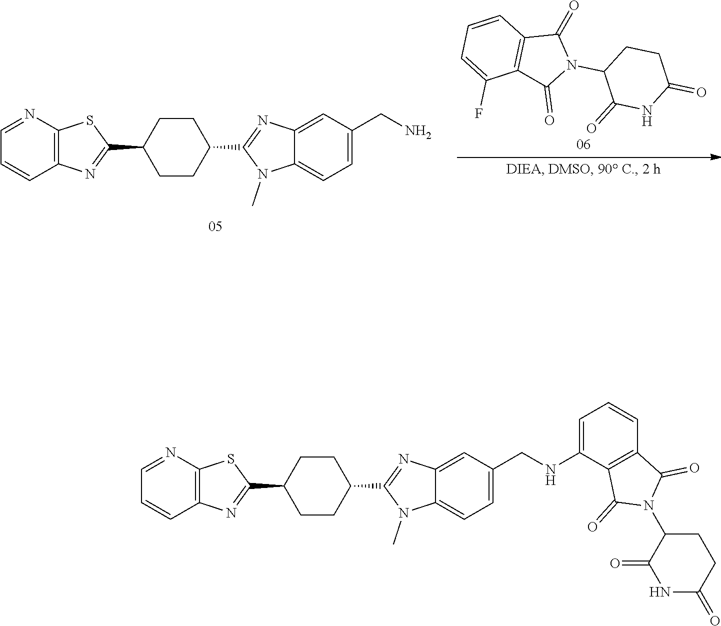 [SMILES: Cn1c2ccc(CN)cc2nc1[C@H]1CC[C@H](c2nc3cccnc3s2)CC1 ; O=C1CCC(N2C(=O)c3cccc(F)c3C2=O)C(=O)N1 ; Cn1c2ccc(CNc3cccc4c3C(=O)N(C3CCC(=O)NC3=O)C4=O)cc2nc1[C@H]1CC[C@H](c2nc3cccnc3s2)CC1]

05 (110 mg, 0.291 mmol), 06 (72.4 mg, 0.262 mmol) and N,N-diisopropylethylamine (113 mg, 0.873 mmol) were dissolved in anhydrous dimethyl sulfoxide (3 mL), and the reaction mixture was stirred at 90° C. for 2 h. The mixture was purified by preparative reversed-phase liquid chromatography (C18, 5-100% acetonitrile/water as mobile phase, 0.1% formic acid) to separate the target product as a yellowish-green solid (20.39 mg, 11.0% yield). LC-MS: 634.4 [M+H]⁺, ¹H NMR (400 MHz, DMSO-d₆) δ 11.09 (s, 1H), 8.59 (d, J=3.5 Hz, 1H), 8.35 (d, J=7.2 Hz, 1H), 7.59-7.55 (m, 2H), 7.51-7.44 (m, 2H), 7.26-7.19 (m, 2H), 7.05-6.99 (m, 2H), 5.07 (dd, J=12.9, 5.4 Hz, 1H), 4.62 (d, J=6.0 Hz, 2H), 3.78 (s, 3H), 3.15-3.09 (m, 1H), 2.95-2.84 (m, 1H), 2.62-2.54 (m, 2H), 2.35-2.26 (m, 2H), 2.14-2.02 (m, 4H), 1.92-1.81 (m, 4H).

### Example 74

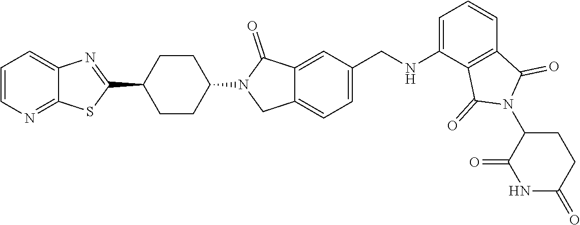 [SMILES: O=C1CCC(N2C(=O)c3cccc(NCc4ccc5c(c4)C(=O)N([C@H]4CC[C@H](c6nc7cccnc7s6)CC4)C5)c3C2=O)C(=O)N1]

### Preparation Steps

### Step 1

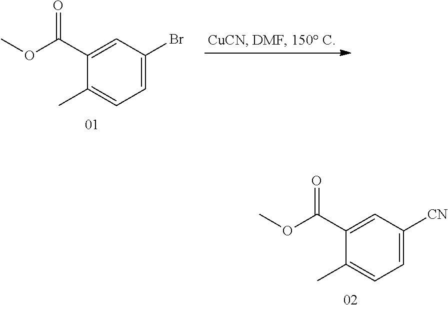

01 (10 g, 43.65 mmol) and cuprous cyanide (5.87 g, 65.48 mmol) were dissolved in N,N-dimethylformamide (100 mL) at room temperature, and the reaction mixture was stirred in an argon atmosphere at 147° C. for 12 h, quenched with ice water, and extracted with ethyl acetate (100 mL×3). The organic phase was washed with saturated brine, dried over anhydrous sodium sulfate, and filtered, and the filtrate was concentrated to dryness by rotary evaporation. The residue was passed through a silica gel column (petroleum ether:ethyl acetate=2:1) to separate the target product as a yellow solid (4.82 g, 63.9% yield).

### Step 2

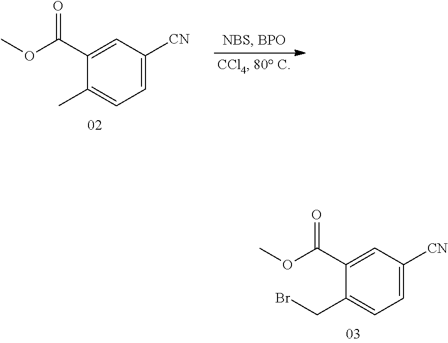

02 (4.82 g, 27.87 mmol) was dissolved in carbon tetrachloride (50 mL) at room temperature, and N-bromosuccinimide (5 g, 28.15 mmol) and dibenzoyl peroxide (675 mg, 2.79 mmol) were added at room temperature. The reaction mixture was stirred in an argon atmosphere at 80° C. for 12 h, quenched with water (50 mL), and extracted with ethyl acetate (100 mL×3). The organic phase was washed with saturated brine, dried over anhydrous sodium sulfate, and filtered, and the filtrate was concentrated to dryness by rotary evaporation. The residue was passed through a silica gel column (petroleum ether:ethyl acetate=2:1) to separate the target product as a white solid (4.45 g, 64.9% yield).

### Step 3

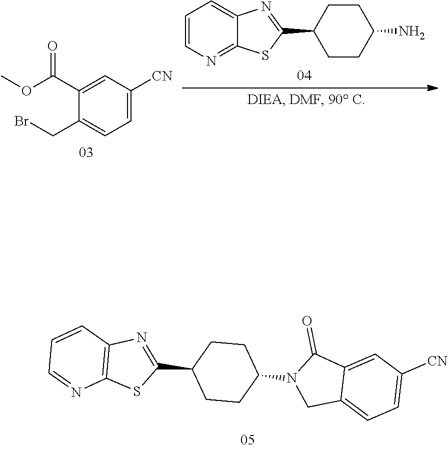

03 (200 mg, 0.78 mmol) and 04 (220 mg, 0.94 mmol) were dissolved in N,N-dimethylformamide (12 mL) at room temperature, and N,N-diisopropylethylamine (305 mg, 2.36 mmol) was added at room temperature. The reaction mixture was stirred in an argon atmosphere at 90° C. for 3 h, quenched with water (50 mL), and extracted with ethyl acetate (100 mL×3). The organic phase was washed with saturated brine, dried over anhydrous sodium sulfate, and filtered, and the filtrate was concentrated to dryness by rotary evaporation. The residue was passed through a silica gel column (methanol:dichloromethane=1:10) to separate the target product as a white solid (83 mg, 28.4% yield). LC-MS: 375.1 [M+H]⁺.

### Step 4

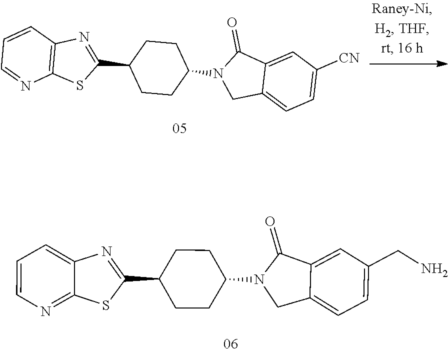 [SMILES: N#Cc1ccc2c(c1)C(=O)N([C@H]1CC[C@H](c3nc4cccnc4s3)CC1)C2 ; NCc1ccc2c(c1)C(=O)N([C@H]1CC[C@H](c3nc4cccnc4s3)CC1)C2]

05 (65 mg, 0.17 mmol) was dissolved in tetrahydrofuran (15 mL) at room temperature, and Raney-Ni was added. The reaction mixture was stirred in a hydrogen atmosphere at room temperature for 12 h and filtered, and the filtrate was concentrated to dryness by rotary evaporation to give the target product as a yellow solid (52 mg, 80.9% yield). LC-MS: 379.2 [M+H]⁺.

### Step 5

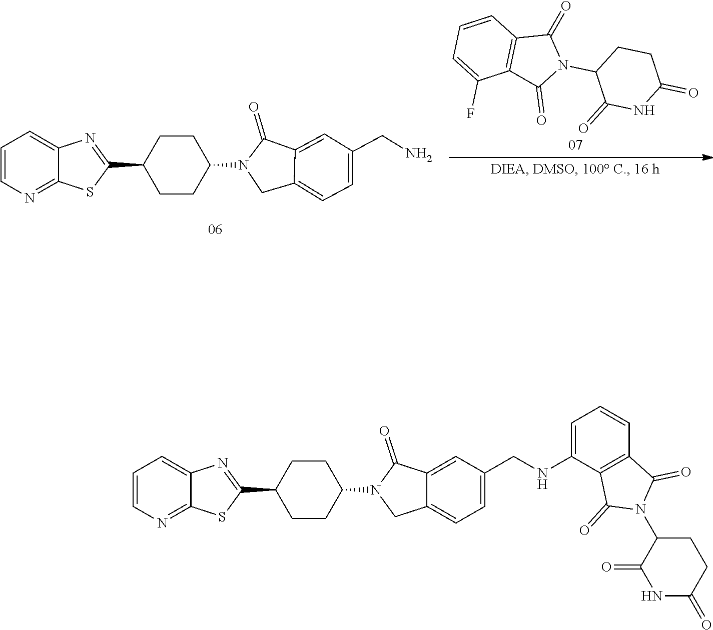 [SMILES: NCc1ccc2c(c1)C(=O)N([C@H]1CC[C@H](c3nc4cccnc4s3)CC1)C2 ; O=C1CCC(N2C(=O)c3cccc(F)c3C2=O)C(=O)N1 ; O=C1CCC(N2C(=O)c3cccc(NCc4ccc5c(c4)C(=O)N([C@H]4CC[C@H](c6nc7cccnc7s6)CC4)C5)c3C2=O)C(=O)N1]

06 (45 mg, 0.12 mmol) and 07 (26 mg, 0.10 mmol) were dissolved in dimethyl sulfoxide (1 mL) at room temperature, and N,N-diisopropylethylamine (46 mg, 0.36 mmol) was added at room temperature. The reaction mixture was stirred at 110° C. for 12 h and purified by preparative chromatography to separate the target product as a green solid (4.01 mg, 5.3% yield). LC-MS: 635.3 [M+H]⁺. ¹H NMR (400 MHz, DMSO-d₆) δ 11.09 (s, 1H), 8.58 (d, J=4.6 Hz, 1H), 8.35 (d, J=8.2 Hz, 1H), 7.70 (s, 1H), 7.63 (d, J=7.8 Hz, 1H), 7.60-7.54 (m, 2H), 7.52-7.46 (m, 1H), 7.38 (d, J=6.0 Hz, 1H), 7.02 (d, J=6.9 Hz, 1H), 6.95 (d, J=8.4 Hz, 1H), 5.07 (dd, J=12.9, 5.1 Hz, 1H), 4.67 (d, J=6.3 Hz, 2H), 4.46 (s, 2H), 4.14 (s, 1H), 3.21-3.18 (m, 1H), 2.95-2.84 (m, 1H), 2.65 (d, J=14.3 Hz, 2H), 2.31 (s, 2H), 2.07 (s, 1H), 1.89 (d, J=9.9 Hz, 2H), 1.82 (s, 4H).

### Example 75

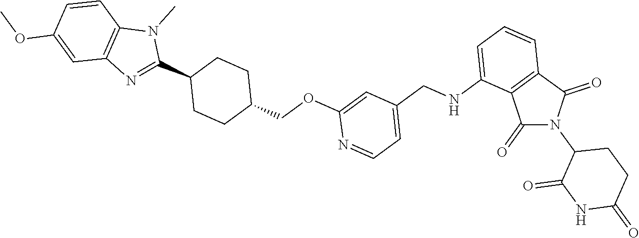 [SMILES: COc1ccc2c(c1)nc([C@H]1CC[C@H](COc3cc(CNc4cccc5c4C(=O)N(C4CCC(=O)NC4=O)C5=O)ccn3)CC1)n2C]

### Preparation Steps

### Step 1

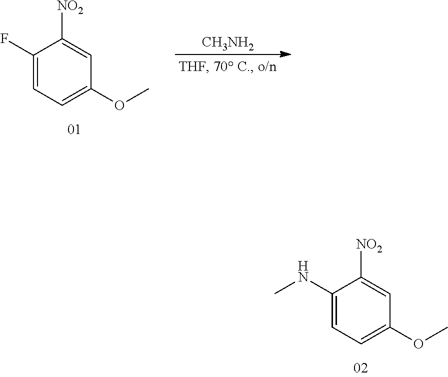

Starting material 01 (5 g, 29 mmol) and methylamine (2.7 g, 88 mmol) was dissolved in tetrahydrofuran (100 mL), and then the reaction mixture was stirred at 70° C. for 16 h. After the reaction was completed, the reaction mixture was directly concentrated to dryness by rotary evaporation to give the target product as a red solid (3.8 g, crude).

### Step 2

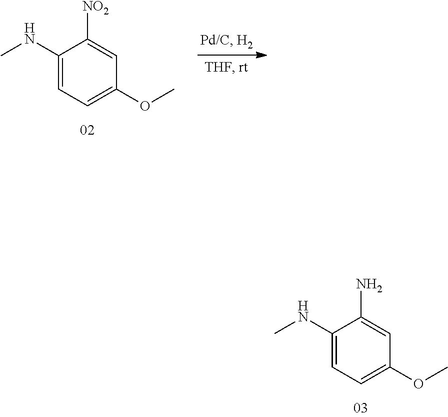

02 (4.45 g, 24.4 mmol) was dissolved in tetrahydrofuran (100 mL) at room temperature, and wet palladium on carbon (1 g) was added. The reaction mixture was purged with hydrogen twice, stirred in a hydrogen atmosphere at room temperature for 16 h, and filtered. The filtrate was concentrated to dryness by rotary evaporation, and the residue was passed through a silica gel column (petroleum ether:ethyl acetate=100:0 to 45:55) to separate the target product as an orange solid (670 mg, 18.0% yield). LC-MS: 153.1 [M+H]⁺.

### Step 3

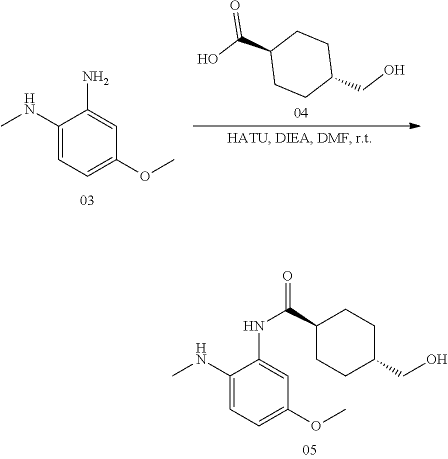

03 (650 mg, 4.27 mmol), 04 (676 mg, 4.27 mmol) and N,N-diisopropylethylamine (1.65 g, 12.8 mmol) were dissolved in N,N-dimethylformamide (10 mL) at room temperature, and (7-azabenzotriazole)-tetramethyluronium hexafluorophosphate (1.95 g, 5.12 mmol) was added with stirring at room temperature. The reaction mixture was stirred at room temperature for 16 h. Water (100 mL) was added, followed by extraction with ethyl acetate (50 mL×3). The organic phase was washed with saturated brine (100 mL), dried over anhydrous sodium sulfate, and filtered, and the filtrate was concentrated to dryness by rotary evaporation. The residue was purified through a silica gel column (dichloromethane:methanol=10:1) to give the target product as a pink solid (2 g, crude). LC-MS: 293.2 [M+H]⁺.

### Step 4

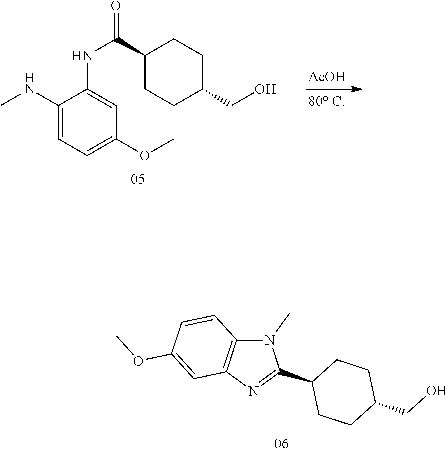 [SMILES: "AcOH" ; CNc1ccc(OC)cc1NC(=O)[C@H]1CC[C@H](CO)CC1 ; COc1ccc2c(c1)nc([C@H]1CC[C@H](CO)CC1)n2C]

05 (2 g, crude) was dissolved in acetic acid (20 mL) at room temperature, and the reaction mixture was stirred at 80° C. for 4 h. The solvent was removed by rotary evaporation. The residue was purified through a reversed-phase column (C18, water:acetonitrile=100:0 to 50:50, 0.1% formic acid) to give the target product as a pale yellow solid (600 mg, 51.2% yield over two steps). LC-MS: 275.1 [M+H]⁺.

### Step 5

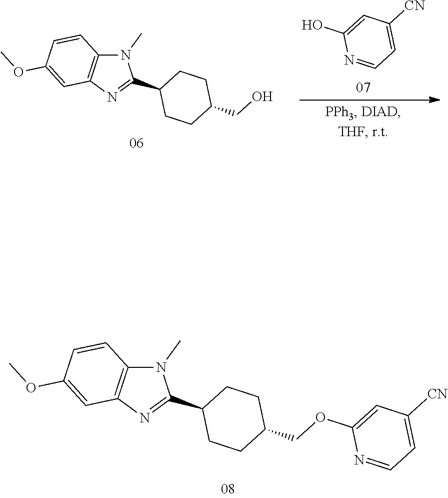 [SMILES: COc1ccc2c(c1)nc([C@H]1CC[C@H](CO)CC1)n2C ; N#Cc1ccnc(O)c1 ; COc1ccc2c(c1)nc([C@H]1CC[C@H](COc3cc(C#N)ccn3)CC1)n2C]

06 (300 mg, 1.09 mmol), 07 (131 mg, 1.09 mmol) and triphenylphosphine (344 mg, 1.31 mmol) were dissolved in anhydrous tetrahydrofuran (20 mL) at room temperature, and diisopropyl azodicarboxylate (265 mg, 1.31 mmol) was added slowly at room temperature. The reaction mixture was stirred at room temperature for 16 h. Water (50 mL) was added, followed by extraction with ethyl acetate (50 mL×3). The organic phase was washed with saturated brine (50 mL), dried over anhydrous sodium sulfate, and filtered, and the filtrate was concentrated to dryness by rotary evaporation. The residue was purified through a reversed-phase column (C18, water:acetonitrile=100:0 to 30:70, 0.1% formic acid) to give the target product as a yellow solid (350 mg, 85% yield). LC-MS: 377.1 [M+H]⁺.

### Step 6

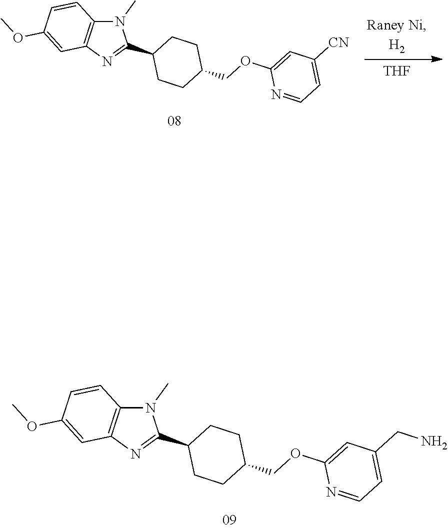 [SMILES: COc1ccc2c(c1)nc([C@H]1CC[C@H](COc3cc(C#N)ccn3)CC1)n2C ; COc1ccc2c(c1)nc([C@H]1CC[C@H](COc3cc(CN)ccn3)CC1)n2C ; "THF"]

08 (150 mg, 0.39 mmol) was dissolved in tetrahydrofuran (10 mL) at room temperature, and Raney nickel (100 mg) was added at room temperature. The reaction mixture was stirred in a hydrogen atmosphere at room temperature for 6 h and filtered, and the filtrate was concentrated to dryness by rotary evaporation to give the target product as a colorless sticky liquid (150 mg, crude). LC-MS: 381.1 [M+H]⁺.

### Step 7

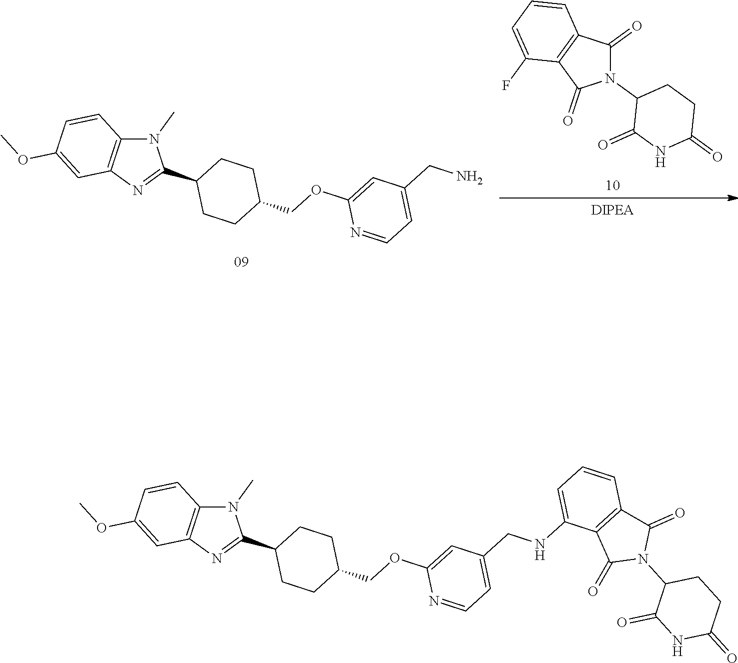 [SMILES: COc1ccc2c(c1)nc([C@H]1CC[C@H](COc3cc(CN)ccn3)CC1)n2C ; O=C1CCC(N2C(=O)c3cccc(F)c3C2=O)C(=O)N1 ; COc1ccc2c(c1)nc([C@H]1CC[C@H](COc3cc(CNc4cccc5c4C(=O)N(C4CCC(=O)NC4=O)C5=O)ccn3)CC1)n2C]

09 (70 mg, 0.184 mmol), 10 (50.8 mg, 0.184 mmol) and N,N-diisopropylethylamine (71.3 mg, 0.552 mmol) were dissolved in dimethyl sulfoxide (5 mL) at room temperature. The reaction mixture was stirred at 80° C. for 16 h. The mixture was cooled to 20° C. and then purified by preparative chromatography (C18, 5-100% acetonitrile/water as mobile phase, 0.1% formic acid) to separate the target product as a yellow solid (24.43 mg, 20.9% yield). LC-MS: 637.0 [M+H]⁺. ¹H NMR (400 MHz, CDCl₃) δ 8.12 (d, J=5.3 Hz, 1H), 8.06 (s, 1H), 7.49-7.43 (m, 1H), 7.38 (s, 1H), 7.23 (d, J=8.9 Hz, 1H), 7.16 (d, J=7.1 Hz, 1H), 6.97 (d, J=7.8 Hz, 1H), 6.84 (d, J=5.3 Hz, 1H), 6.75 (dd, J=16.6, 7.7 Hz, 3H), 4.95 (dd, J=12.2, 5.3 Hz, 1H), 4.49 (d, J=6.1 Hz, 2H), 4.16 (d, J=6.2 Hz, 2H), 3.86 (s, 3H), 3.79 (s, 3H), 2.99-2.68 (m, 4H), 2.18-1.99 (m, 9H), 1.29 (dd, J=18.2, 10.6 Hz, 2H).

### Example 76

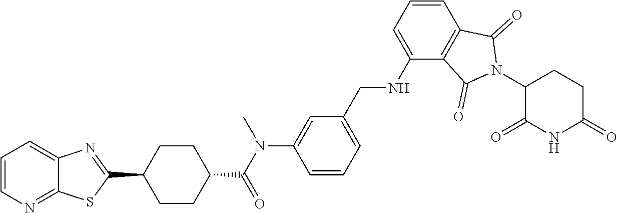 [SMILES: CN(c1cccc(CNc2cccc3c2C(=O)N(C2CCC(=O)NC2=O)C3=O)c1)C(=O)[C@H]1CC[C@H](c2nc3cccnc3s2)CC1]

### Preparation Steps

### Step 1

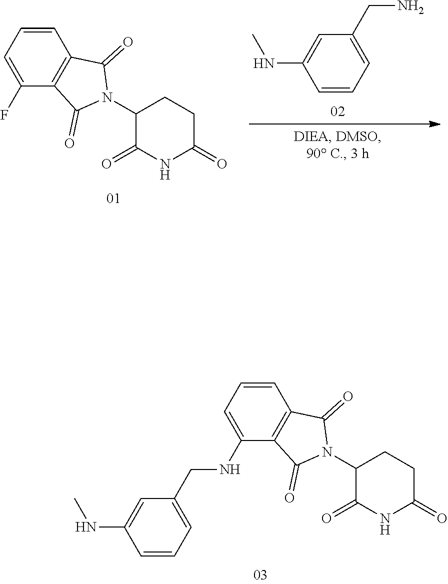

01 (203 mg, 0.734 mmol), 02 (100 mg, 0.734 mmol) and N,N-diisopropylethylamine (284 mg, 2.20 mmol) were dissolved in anhydrous dimethyl sulfoxide (1 mL) at room temperature. The reaction mixture was stirred at 90° C. for 3 h. The mixture was purified through a reversed-phase column (C18, 5-100% acetonitrile/water as mobile phase, 0.1% formic acid) to give the target product as a green solid (94.0 mg, 32.6% yield). LC-MS: 393.3 [M+H]⁺.

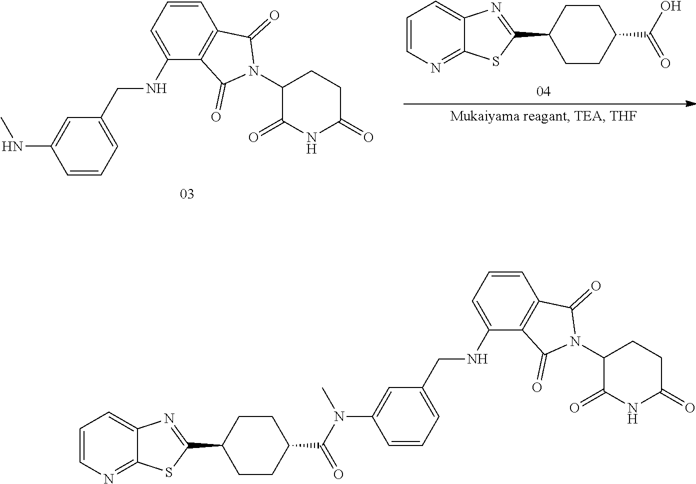 [SMILES: CNc1cccc(CNc2cccc3c2C(=O)N(C2CCC(=O)NC2=O)C3=O)c1 ; O=C(O)[C@H]1CC[C@H](c2nc3cccnc3s2)CC1 ; CN(c1cccc(CNc2cccc3c2C(=O)N(C2CCC(=O)NC2=O)C3=O)c1)C(=O)[C@H]1CC[C@H](c2nc3cccnc3s2)CC1]

To a sealed tube were added at room temperature 03 (54.0 mg, 0.138 mmol), 04 (36.2 mg, 0.138 mmol), triethylamine (33.5 mg, 0.331 mmol), 2-chloro-1-methylpyridinium iodide (42.3 mg, 0.166 mmol) and anhydrous tetrahydrofuran (3 mL), and the reaction mixture was stirred at 70° C. for 16 h. The mixture was purified by preparative chromatography (C18, 10-100% acetonitrile/water as mobile phase, 0.1% formic acid) to separate the target product as a green solid (11.95 mg, 13.6% yield). LC-MS: 637.4 [M+H]⁺, ¹H NMR (400 MHz, DMSO-d₆) δ 11.08 (s, 1H), 8.55 (dd, J=4.6, 1.3 Hz, 1H), 8.29 (dd, J=8.2, 1.1 Hz, 1H), 7.54 (dd, J=8.2, 4.6 Hz, 1H), 7.51-7.42 (m, 3H), 7.40-7.35 (m, 1H), 7.33 (s, 1H), 7.24 (d, J=7.2 Hz, 1H), 6.98 (d, J=8.6 Hz, 1H), 6.90 (d, J=6.9 Hz, 1H), 5.04 (dd, J=12.9, 5.3 Hz, 1H), 4.60 (d, J=5.9 Hz, 2H), 3.14 (s, 3H), 3.03 (t, J=12.0 Hz, 1H), 2.91-2.81 (m, 1H), 2.61-2.53 (m, 2H), 2.13-1.96 (m, 4H), 1.68-1.49 (m, 4H), 1.15-0.97 (m, 2H).

### Example 77

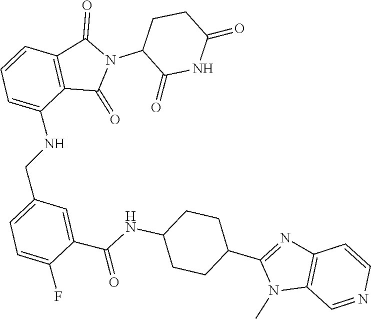 [SMILES: Cn1c(C2CCC(NC(=O)c3cc(CNc4cccc5c4C(=O)N(C4CCC(=O)NC4=O)C5=O)ccc3F)CC2)nc2ccncc21]

### Preparation Steps

### Step 1

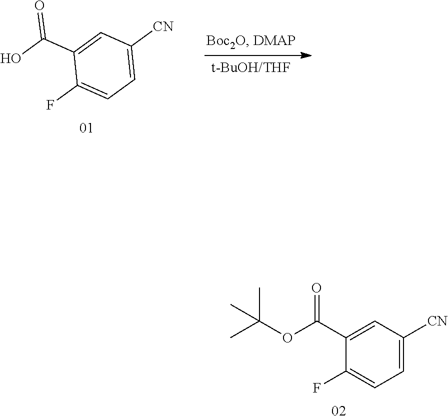 [SMILES: N#Cc1ccc(F)c(C(=O)O)c1 ; CC(C)(C)OC(=O)c1cc(C#N)ccc1F]

In a 100 mL single-neck flask, 01 (500 mg, 3.03 mmol) was dissolved in a mixed solution of tert-butanol (9 mL)/tetrahydrofuran (3 mL), and di-tert-butyl dicarbonate (1.32 g, 6.06 mmol) and 4-dimethylaminopyridine (111 mg, 0.908 mmol) were added. The mixture was reacted at room temperature for 16 h, concentrated under reduced pressure to remove the volatile solvent, then diluted with water (15 mL), and extracted with ethyl acetate (20 mL×3). The organic phase was washed with saturated brine (20 mL×3) and concentrated to dryness by rotary evaporation, and the residue was purified by column chromatography (petroleum ether:ethyl acetate=5:1) to give 02 (430 mg, 64.2% yield, brown solid).

LC-MS: 222.1 [M+H]⁺.

### Step 2

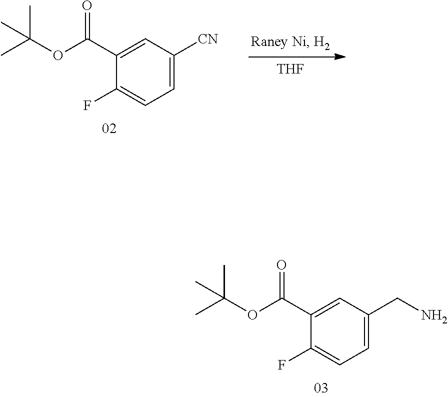 [SMILES: CC(C)(C)OC(=O)c1cc(C#N)ccc1F ; CC(C)(C)OC(=O)c1cc(CN)ccc1F ; "THF"]

In a 100 mL single-neck flask, 02 (200 mg, 0.904 mmol) was dissolved in tetrahydrofuran (10 mL), and Raney nickel (30 mg) was added. The mixture was reacted in a hydrogen atmosphere at room temperature for 3 h and filtered, and the filtrate was concentrated to give 03 (195 mg, 95.8% yield, yellow oil).

LC-MS: 226.1 [M+H]⁺.

### Step 3

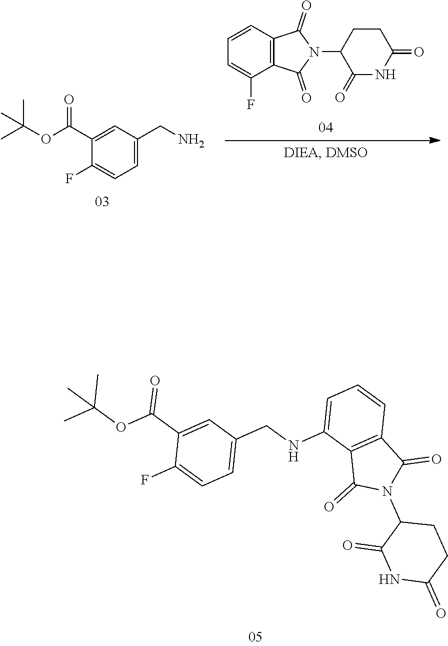 [SMILES: CC(C)(C)OC(=O)c1cc(CN)ccc1F ; O=C1CCC(N2C(=O)c3cccc(F)c3C2=O)C(=O)N1 ; CC(C)(C)OC(=O)c1cc(CNc2cccc3c2C(=O)N(C2CCC(=O)NC2=O)C3=O)ccc1F]

In a 50 mL single-neck flask, 03 (50 mg, 0.222 mmol) was dissolved in dimethyl sulfoxide (1 mL), and 04 (67 mg, 0.244 mmol) and N,N-diisopropylethylamine (57 mg, 0.444 mmol) were added. The mixture was reacted in an argon atmosphere at 120° C. for 16 h, quenched with water (5 mL), and extracted with ethyl acetate (10 mL×3). The organic phase was washed with saturated brine (10 mL×3) and concentrated to dryness by rotary evaporation, and the residue was purified by column chromatography (dichloromethane:methanol=20:1) to give 05 (90 mg, 84.2% yield, brown solid).

LC-MS: 482.1 [M+H]⁺.

### Step 4

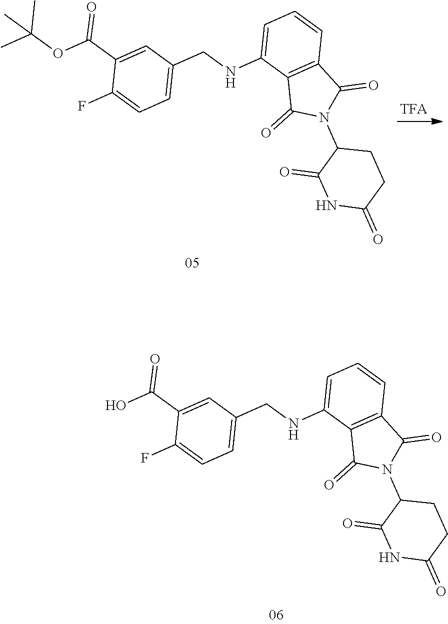 [SMILES: CC(C)(C)OC(=O)c1cc(CNc2cccc3c2C(=O)N(C2CCC(=O)NC2=O)C3=O)ccc1F ; O=C1CCC(N2C(=O)c3cccc(NCc4ccc(F)c(C(=O)O)c4)c3C2=O)C(=O)N1]

In a 50 mL single-neck flask, 05 (40 mg, 0.0831 mmol) was dissolved in dichloromethane (5 mL), and trifluoroacetic acid (1 mL) was added. The mixture was reacted at room temperature for 2 h and concentrated to dryness by rotary evaporation to give crude 06 (38 mg, crude, yellow solid).

LC-MS: 426.2 [M+H]⁺.

### Step 5

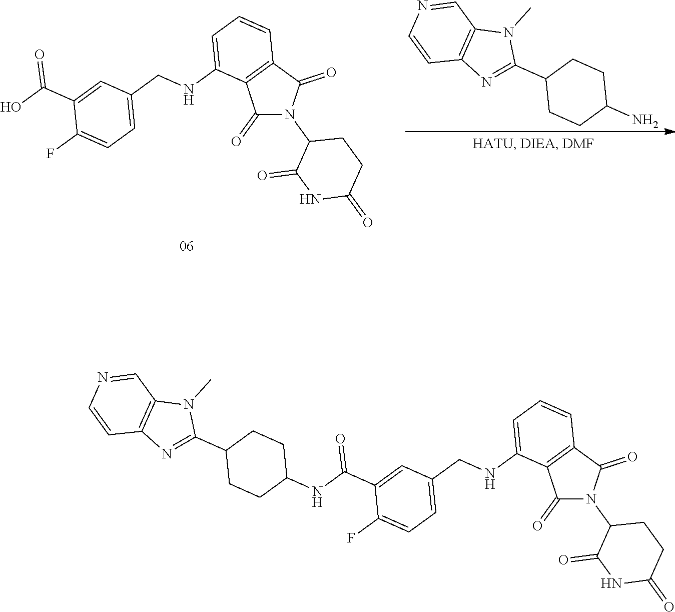 [SMILES: O=C1CCC(N2C(=O)c3cccc(NCc4ccc(F)c(C(=O)O)c4)c3C2=O)C(=O)N1 ; Cn1c(C2CCC(N)CC2)nc2ccncc21 ; Cn1c(C2CCC(NC(=O)c3cc(CNc4cccc5c4C(=O)N(C4CCC(=O)NC4=O)C5=O)ccc3F)CC2)nc2ccncc21]

Crude 06 (38 mg, 0.0831 mmol) was dissolved in N,N-dimethylformamide (1 mL) at room temperature, and HATU (47 mg, 0.125 mmol), N,N-diisopropylethylamine (32 mg, 0.249 mmol) and 07 (19 mg, 0.0831 mmol) were added successively. The above mixture was stirred at room temperature for 2 h. The reaction mixture was purified by preparative high performance liquid chromatography (hydrochloric acid) to give the target product (9.55 mg, 18.2% yield, yellow solid).

LC-MS: 638.3 [M+H]⁺.

¹H NMR (400 MHz, DMSO-d6) δ 11.10 (s, 1H), 9.49 (s, 1H), 8.57 (s, 1H), 8.33 (d, J=7.8 Hz, 1H), 8.20-8.13 (m, 1H), 7.58 (d, J=4.7 Hz, 1H), 7.51 (d, J=7.5 Hz, 2H), 7.32 (s, 1H), 7.25 (t, J=9.0 Hz, 1H), 7.04 (d, J=6.8 Hz, 1H), 6.98 (d, J=8.6 Hz, 1H), 5.07 (dd, J=12.7, 5.2 Hz, 1H), 4.57 (s, 2H), 4.04 (d, J=4.2 Hz, 3H), 3.24-3.14 (m, 1H), 2.95-2.83 (m, 1H), 2.65-2.53 (m, 2H), 2.15-1.92 (m, 6H), 1.84-1.71 (m, 2H), 1.61-1.46 (m, 2H).

### Example 78

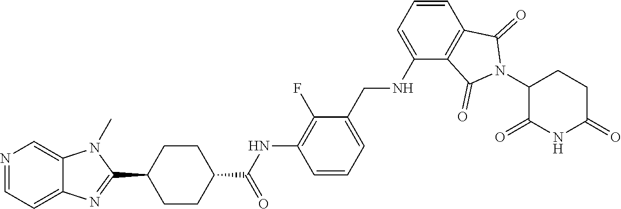 [SMILES: Cn1c2cnccc2nc1[C@H]1CC[C@H](C(=O)Nc2cccc(CNc3cccc4c3C(=O)N(C3CCC(=O)NC3=O)C4=O)c2F)CC1]

### Preparation Steps

### Step 1

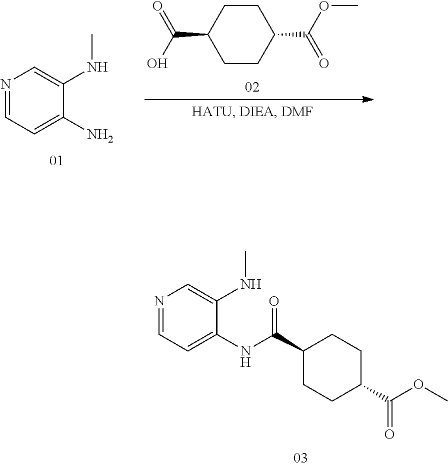

01 (1 g, 8.13 mmol), 02 (1.36 g, 7.32 mmol) and N,N-diisopropylethylamine (2.1 g, 16.62 mmol) were dissolved in N,N-dimethylformamide (12 mL) at room temperature, and (7-azabenzotriazole)-tetramethyluronium hexafluorophosphate (3.71 g, 9.76 mmol) was added with stirring at room temperature. The reaction mixture was stirred at room temperature for 2 h and passed through a reversed-phase column (C18, water:acetonitrile=100:0 to 35:65, 0.1% formic acid) to separate the target product as a white solid (1.7 g, 79.8% yield). LC-MS: 292.1 [M+H]⁺.

### Step 2

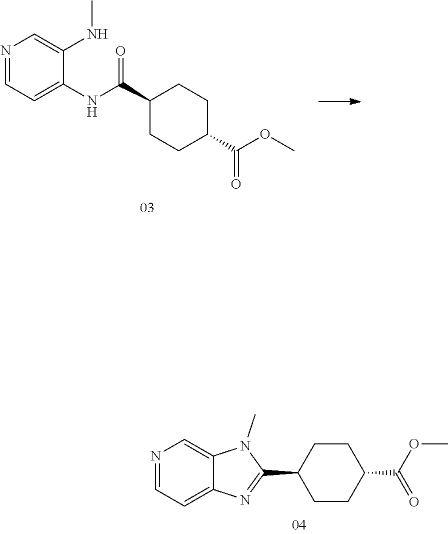 [SMILES: CNc1cnccc1NC(=O)[C@H]1CC[C@H](C(=O)OC)CC1 ; COC(=O)[C@H]1CC[C@H](c2nc3ccncc3n2C)CC1]

03 (1.7 g, 5.84 mmol) was dissolved in acetic acid (6 mL) at room temperature, and the reaction mixture was stirred in an argon atmosphere at 70° C. for 2 h. The mixture was concentrated to dryness by rotary evaporation to give the target product as a yellow oil (1.5 g, 94.0% yield). LC-MS: 274.2 [M+H]⁺.

### Step 3

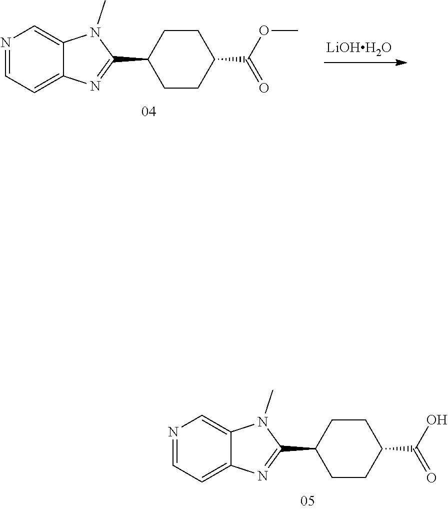

04 (1.5 g, 5.49 mmol) was dissolved in tetrahydrofuran (5 mL) and water (5 mL) at room temperature, and lithium hydroxide monohydrate (231 mg, 5.49 mmol) was added at room temperature. The reaction mixture was stirred at room temperature for 3 h, concentrated by rotary evaporation to remove tetrahydrofuran, adjusted to a pH value less than 7, and concentrated to dryness by rotary evaporation to give the target product as a white solid (1.3 g, 91.4% yield). LC-MS: 260.2 [M+H]⁺.

### Step 4

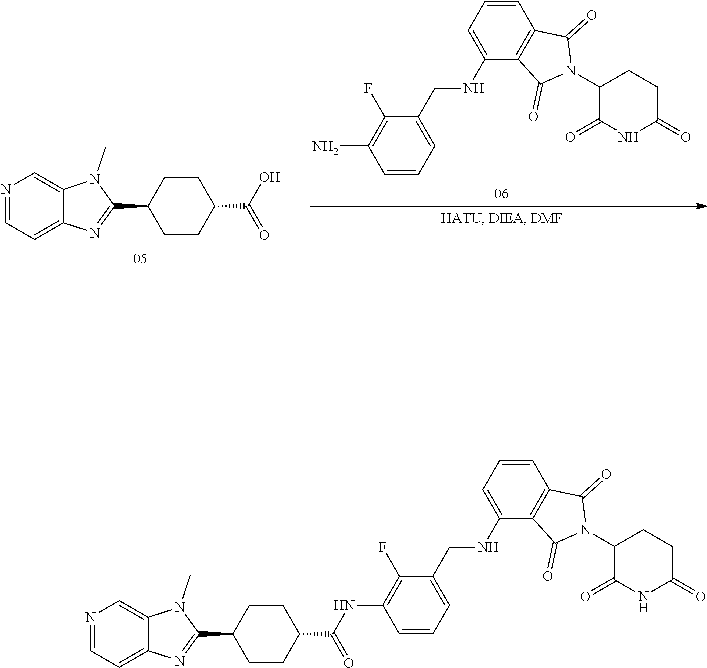 [SMILES: Cn1c2cnccc2nc1[C@H]1CC[C@H](C(=O)O)CC1 ; Nc1cccc(CNc2cccc3c2C(=O)N(C2CCC(=O)NC2=O)C3=O)c1F ; Cn1c2cnccc2nc1[C@H]1CC[C@H](C(=O)Nc2cccc(CNc3cccc4c3C(=O)N(C3CCC(=O)NC3=O)C4=O)c2F)CC1]

05 (118 mg, 0.45 mmol), 06 (150 mg, 0.37 mmol), N-methylimidazole (62 mg, 0.74 mmol) and N,N,N′,N′-tetramethylchloroformamidinium hexafluorophosphate (135 mg, 0.48 mmol) were dissolved in acetonitrile (2 mL) at room temperature. The reaction mixture was stirred at room temperature for 2 h. The mixture was purified by preparative chromatography (C18, 5-100% acetonitrile/water as mobile phase, 0.03% hydrochloric acid) followed by lyophilization to give the target product as a yellow solid (30.12 mg, 9.93% yield). LC-MS: [M+H]⁺ 638.4. 1H NMR (400 MHz, DMSO-d₆) δ 11.10 (s, 1H), 9.77 (s, 1H), 9.50 (s, 1H), 8.56 (d, J=6.4 Hz, 1H), 8.16 (d, J=6.5 Hz, 1H), 7.78 (s, 1H), 7.58-7.52 (m, 1H), 7.16-7.06 (m, 3H), 7.04 (dd, J=9.8, 8.0 Hz, 2H), 5.11-5.04 (m, 1H), 4.64 (s, 2H), 4.04 (s, 3H), 2.89 (s, 2H), 2.73 (s, 1H), 2.64-2.56 (m, 2H), 2.10-1.98 (m, 5H), 1.77-1.67 (m, 4H).

### Example 79

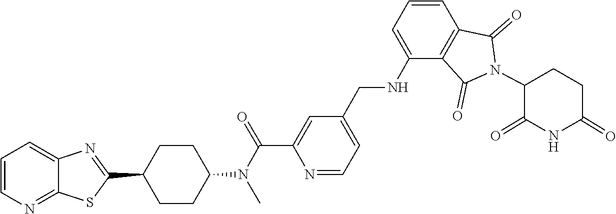 [SMILES: CN(C(=O)c1cc(CNc2cccc3c2C(=O)N(C2CCC(=O)NC2=O)C3=O)ccn1)[C@H]1CC[C@H](c2nc3cccnc3s2)CC1]

### Preparation Steps

### Step 1

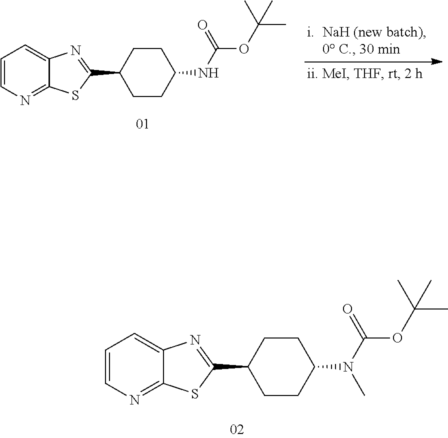 [SMILES: CC(C)(C)OC(=O)N[C@H]1CC[C@H](c2nc3cccnc3s2)CC1 ; CN(C(=O)OC(C)(C)C)[C@H]1CC[C@H](c2nc3cccnc3s2)CC1]

01 (30 mg, 0.09 mmol) was dissolved in N,N-dimethylformamide (3 mL) at room temperature, and sodium hydride (60%) (18 mg, 0.451 mmol) was added at 0° C. The reaction mixture was stirred at 0° C. for 1 h, then iodomethane (19 mg, 0.135 mmol) was added, and the reaction mixture was stirred at room temperature for 16 h, quenched with ammonium chloride (10 mL), and extracted with ethyl acetate (10 mL×3). The organic phase was washed with saturated brine, dried over anhydrous sodium sulfate, and concentrated to dryness by rotary evaporation to give the target product as a yellow oily liquid (25 mg, 84.3% yield). LC-MS: 348.3 [M+H]⁺.

### Step 2

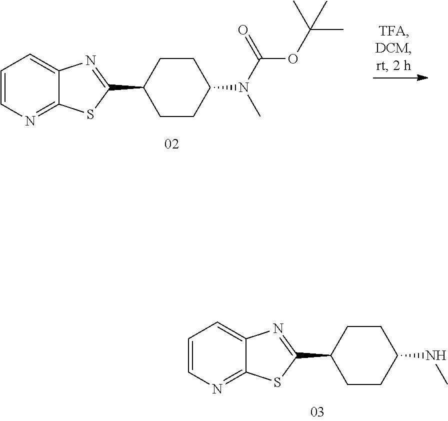 [SMILES: CN(C(=O)OC(C)(C)C)[C@H]1CC[C@H](c2nc3cccnc3s2)CC1 ; CN[C@H]1CC[C@H](c2nc3cccnc3s2)CC1]

02 (24 mg, 0.0689 mmol) was dissolved in dichloromethane (2 mL) at room temperature, and trifluoroacetic acid (0.3 mL) was added at room temperature. The reaction mixture was stirred at room temperature for 2 h and concentrated to dryness by rotary evaporation to give the target product as a yellow oily liquid (19 mg, 83.4% yield). LC-MS: 247.1 [M+H]⁺.

### Step 3

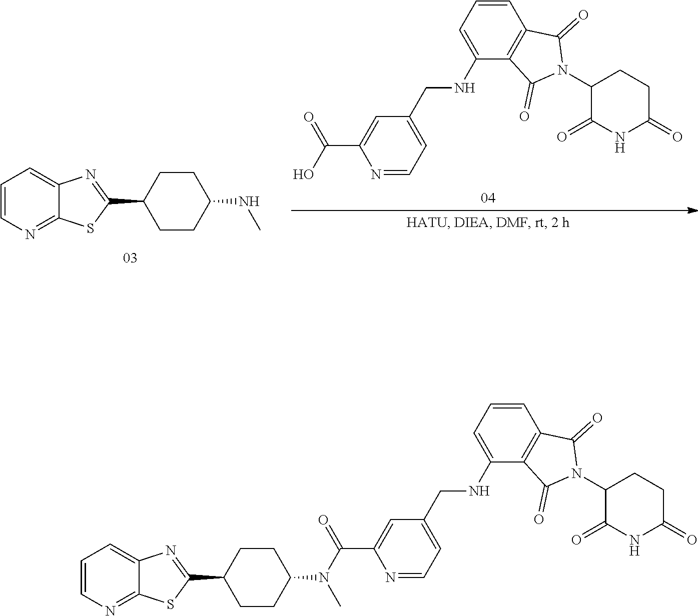 [SMILES: CN[C@H]1CC[C@H](c2nc3cccnc3s2)CC1 ; O=C1CCC(N2C(=O)c3cccc(NCc4ccnc(C(=O)O)c4)c3C2=O)C(=O)N1 ; CN(C(=O)c1cc(CNc2cccc3c2C(=O)N(C2CCC(=O)NC2=O)C3=O)ccn1)[C@H]1CC[C@H](c2nc3cccnc3s2)CC1]

03 (10 mg, 0.0381 mmol), 04 (15 mg, 0.0381 mmol) and N,N-diisopropylethylamine (15 mg, 0.114 mmol) were dissolved in N,N-dimethylformamide (1 mL) at room temperature, and (7-azabenzotriazole)-tetramethyluronium hexafluorophosphate (22 mg, 0.0572 mmol) was added with stirring at room temperature. The reaction mixture was stirred at room temperature for 2 h. The mixture was purified by preparative chromatography to separate the target product as a yellow solid (1 mg, 9.3% yield). LC-MS: 638.1 [M+H]⁺.

¹H NMR (400 MHz, DMSO-d₆) δ 11.12 (s, 1H), 9.42 (s, 1H), 8.78-8.42 (m, 3H), 8.27-8.08 (m, 2H), 7.86 (t, J=7.9 Hz, 1H), 7.76 (t, J=5.9 Hz, 1H), 7.55 (dd, J=19.2, 7.9 Hz, 2H), 5.56 (s, 2H), 5.13 (dd, J=12.9, 5.3 Hz, 1H), 4.14-3.99 (m, 3H), 3.99-3.87 (m, 1H), 3.23-3.10 (m, 1H), 2.96-2.84 (m, 1H), 2.68-2.55 (m, 2H), 2.16-1.94 (m, 5H), 1.93-1.60 (m, 4H).

### Example 80

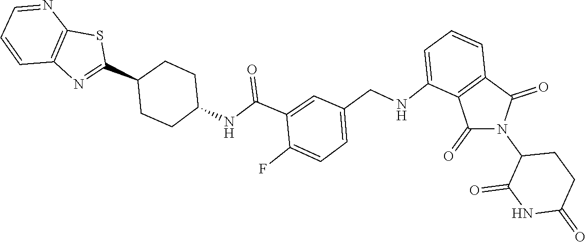 [SMILES: O=C1CCC(N2C(=O)c3cccc(NCc4ccc(F)c(C(=O)N[C@H]5CC[C@H](c6nc7cccnc7s6)CC5)c4)c3C2=O)C(=O)N1]

### Preparation Steps

### Step 1

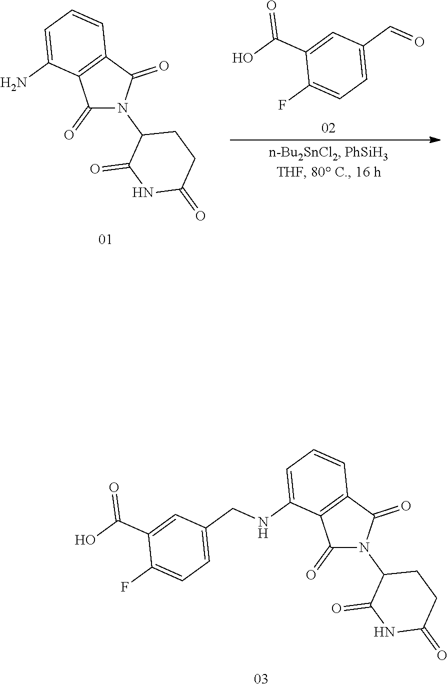 [SMILES: Nc1cccc2c1C(=O)N(C1CCC(=O)NC1=O)C2=O ; O=Cc1ccc(F)c(C(=O)O)c1 ; O=C1CCC(N2C(=O)c3cccc(NCc4ccc(F)c(C(=O)O)c4)c3C2=O)C(=O)N1]

To a 20 mL microwave tube were added 01 (135 mg, 0.50 mmol), 02 (100 mg, 0.60 mmol), dibutyltin chloride (180 mg, 0.60 mmol), triphenylsilane (54 mg, 0.40 mmol) and THF (3 mL), and the mixture was reacted in the sealed tube at 80° C. for 16 h, quenched with water, and extracted with ethyl acetate. The organic phase was washed with saturated brine, dried over anhydrous sodium sulfate, and concentrated to dryness by rotary evaporation, and the residue was purified by preparative thin layer chromatography (dichloromethane:methanol=10:1) to give the product as a yellow solid (75 mg, 35.7% yield). LC-MS: 426.1 [M+H]⁺.

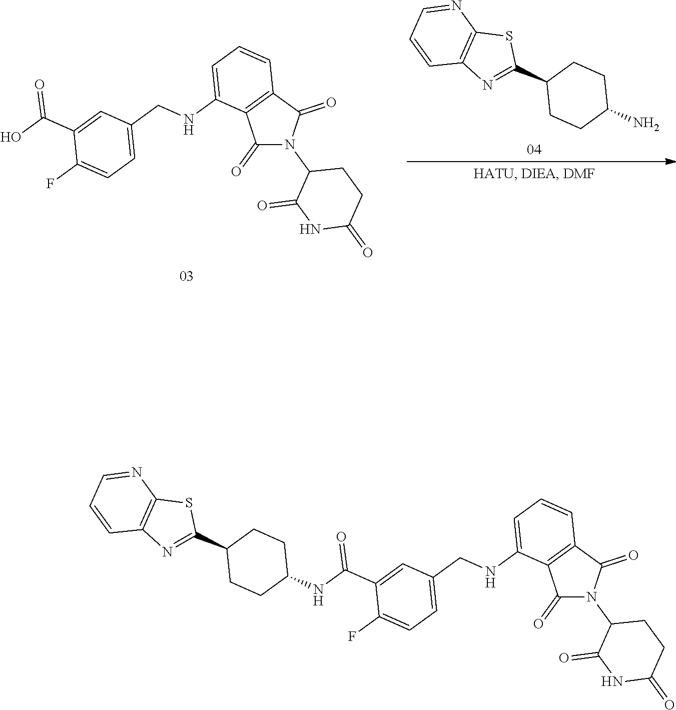 [SMILES: O=C1CCC(N2C(=O)c3cccc(NCc4ccc(F)c(C(=O)O)c4)c3C2=O)C(=O)N1 ; N[C@H]1CC[C@H](c2nc3cccnc3s2)CC1 ; O=C1CCC(N2C(=O)c3cccc(NCc4ccc(F)c(C(=O)N[C@H]5CC[C@H](c6nc7cccnc7s6)CC5)c4)c3C2=O)C(=O)N1]

To a 50 mL single-neck flask were added 03 (75 mg, 0.18 mmol), 04 (40 mg, 0.18 mmol), HATU (98 mg, 0.26 mmol), DIEA (68 mg, 0.53 mmol) and DMF (2 mL), and the mixture was stirred at room temperature for 16 h and purified by preparative reversed-phase chromatography to give the product as a yellow solid (20.52 mg, 18.9% yield). LC-MS: 641.1 [M+H]⁺, ¹HNMR (400 MHz, DMSO-d₆) δ 11.11 (s, 1H), 8.60-8.57 (m, 1H), 8.35 (d, J=8.2 Hz, 1H), 8.29 (d, J=7.6 Hz, 1H), 7.60 (d, J=6.8 Hz, 1H), 7.58-7.54 (m, 1H), 7.52 (d, J=7.5 Hz, 1H), 7.33 (s, 1H), 7.29-7.20 (m, 1H), 7.04 (d, J=7.0 Hz, 1H), 6.98 (d, J=8.6 Hz, 1H), 5.08 (dd, J=12.9, 5.2 Hz, 1H), 4.58 (d, J=5.7 Hz, 2H), 3.84 (d, J=7.6 Hz, 1H), 3.14 (t, J=11.9 Hz, 1H), 2.95-2.83 (m, 1H), 2.58 (dd, J=16.5, 10.2 Hz, 2H), 2.23 (d, J=11.7 Hz, 2H), 2.08-2.00 (m, 3H).

### Example 81

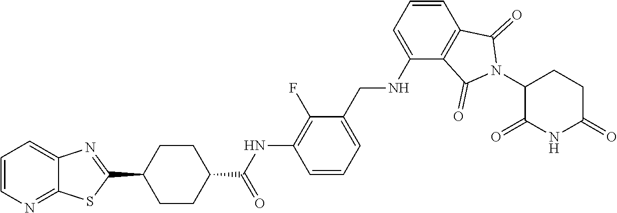 [SMILES: O=C1CCC(N2C(=O)c3cccc(NCc4cccc(NC(=O)[C@H]5CC[C@H](c6nc7cccnc7s6)CC5)c4F)c3C2=O)C(=O)N1]

### Preparation Steps

### Step 1

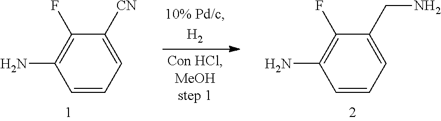 [SMILES: N#Cc1cccc(N)c1F ; NCc1cccc(N)c1F]

Compound 1 (1.0 g, 7.35 mmol) was dissolved in methanol (30 mL) at room temperature, and concentrated hydrochloric acid (1.0 mL) was added, followed by addition of 10% Pd/C (200 mg). The mixture was stirred in a hydrogen atmosphere at room temperature for 48 h. The reaction mixture was concentrated under reduced pressure to give a crude product, which was then purified by reversed-phase chromatography to give compound 2 (900 mg, 83.73% yield). LC-MS: 141.1 [M+H]⁺.

### Step 2

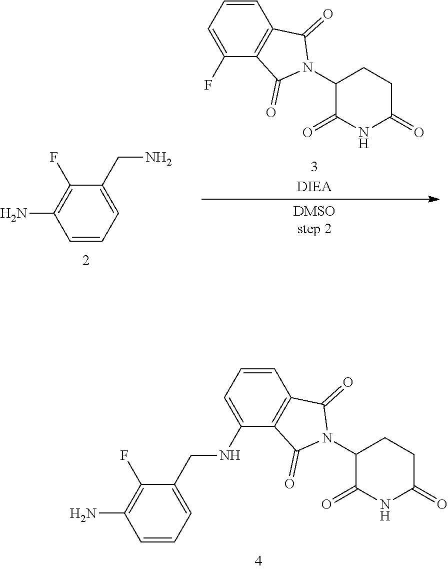 [SMILES: O=C1CCC(N2C(=O)c3cccc(F)c3C2=O)C(=O)N1 ; NCc1cccc(N)c1F ; Nc1cccc(CNc2cccc3c2C(=O)N(C2CCC(=O)NC2=O)C3=O)c1F]

Compound 2 (500 mg, 3.51 mmol) was dissolved in dimethyl sulfoxide (10 mL) at room temperature, and compound 3 (940 mg, 3.51 mmol) and N,N-diisopropylethylamine (900 mg, 7 mmol) were added successively. In a nitrogen atmosphere, the reaction mixture was heated to 100° C. and stirred for 3 h. The reaction mixture was purified by reversed-phase chromatography to give compound 4 (650 mg, 46.4% yield). LC-MS: 397.1 [M+H]⁺.

### Step 3

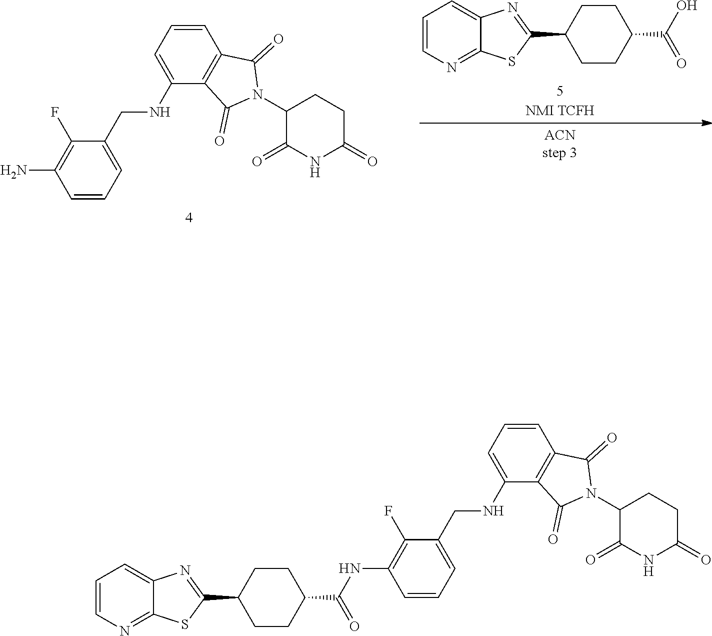 [SMILES: Nc1cccc(CNc2cccc3c2C(=O)N(C2CCC(=O)NC2=O)C3=O)c1F ; O=C(O)[C@H]1CC[C@H](c2nc3cccnc3s2)CC1 ; O=C1CCC(N2C(=O)c3cccc(NCc4cccc(NC(=O)[C@H]5CC[C@H](c6nc7cccnc7s6)CC5)c4F)c3C2=O)C(=O)N1]

Compound 4 (100 mg, 0.25 mmol), compound 5 (66 mg, 0.25 mmol), MNI (41 mg, 0.5 mmol) and N,N,N,N-tetramethylchloroformamidinium hexafluorophosphate (144 mg, 0.25 mmol) were dissolved in acetonitrile at room temperature. The mixture was stirred at room temperature for 3 h. The reaction mixture was purified by reversed-phase chromatography to give the compound PRJ2-3168 (7.03 mg, 4.36% yield). LC-MS: 641.1 [M+H]⁺.

¹H NMR (400 MHz, DMSO-d₆) δ 11.11 (s, 1H), 9.71 (s, 1H), 8.58 (dd, J=4.6, 1.4 Hz, 1H), 8.38-8.31 (m, 1H), 7.83-7.73 (m, 1H), 7.61-7.51 (m, 2H), 7.16-7.01 (m, 5H), 5.07 (dd, J=12.8, 5.2 Hz, 1H), 4.64 (d, J=6.2 Hz, 2H), 3.71-3.40 (m, 1H), 3.35-3.28 (m, 1H), 3.24-3.13 (m, 1H), 2.93-2.84 (m, 1H), 2.65-2.52 (m, 4H), 2.30-2.22 (m, 2H), 2.09-1.60 (m, 8H).

### Example 82

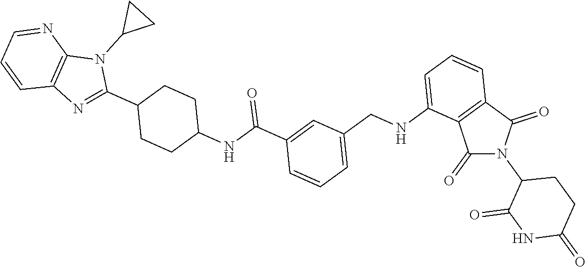 [SMILES: O=C1CCC(N2C(=O)c3cccc(NCc4cccc(C(=O)NC5CCC(c6nc7cccnc7n6C6CC6)CC5)c4)c3C2=O)C(=O)N1]

### Preparation Steps

### Step 1

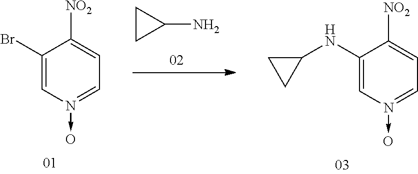

To a 100 mL single-neck flask were added 01 (1 g, 4.57 mmol), 02 (1.31 g, 22.95 mmol) and tetrahydrofuran (20 mL), and the mixture was reacted at 60° C. for 2 h, quenched with water (20 mL), and extracted with ethyl acetate (20 mL×2). The organic phase was washed with saturated brine (20 mL×3) and concentrated to dryness by rotary evaporation, and the residue was purified by column chromatography (ethyl acetate:petroleum ether=1:1) to give the target product as a yellow solid (740 mg, 83.0% yield). LC-MS: 196.1 [M+H]⁺.

### Step 2

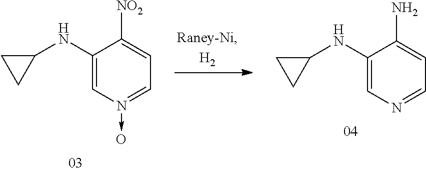 [SMILES: O=[N+]([O-])c1cc[n+]([O-])cc1NC1CC1 ; Nc1ccncc1NC1CC1]

To a 100 mL single-neck flask were added 03 (740 mg, 3.79 mmol), Raney nickel (300 mg) and tetrahydrofuran (40 mL), and the mixture was reacted in a hydrogen atmosphere at room temperature for 2 h and filtered. The filtrate was concentrated to dryness by rotary evaporation to give the target product as a red solid (500 mg, 88.4% yield). LC-MS: 150.1 [M+H]⁺.

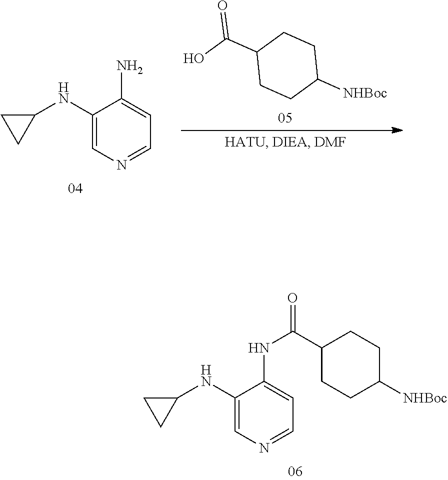 [SMILES: Nc1ccncc1NC1CC1 ; CC(C)(C)OC(=O)NC1CCC(C(=O)O)CC1 ; CC(C)(C)OC(=O)NC1CCC(C(=O)Nc2ccncc2NC2CC2)CC1]

To a 100 mL single-neck flask were added 04 (400 mg, 2.68 mmol), 05 (620 mg, 2.55 mmol), HATU (1.53 g, 4.02 mmol), DIEA (1.04 g, 8.05 mmol) and DMF (20 mL), and the mixture was reacted at room temperature for 2 h, quenched with water (20 mL), and extracted with ethyl acetate (20 mL×2). The organic phase was washed with saturated brine (20 mL×3) and concentrated to dryness by rotary evaporation, and the residue was purified by column chromatography (dichloromethane:methanol=10:1) to give the target product as a yellow solid (400 mg, 39.8% yield). LC-MS: 375.2 [M+H]⁺.

### Step 4

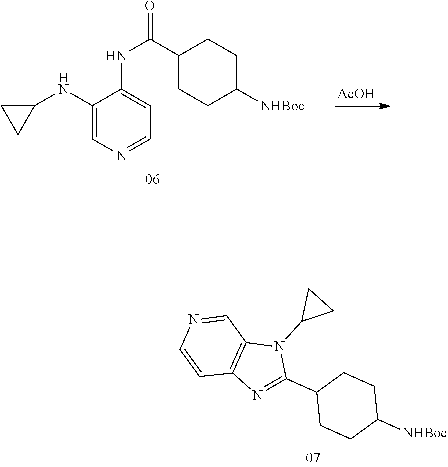 [SMILES: "AcOH" ; CC(C)(C)OC(=O)NC1CCC(C(=O)Nc2ccncc2NC2CC2)CC1 ; CC(C)(C)OC(=O)NC1CCC(c2nc3ccncc3n2C2CC2)CC1]

In a 100 mL single-neck round-bottom flask, 06 (100 mg, 0.267 mmol) was dissolved in acetic acid (5 mL), and the mixture was reacted at 70° C. for 2.5 h and concentrated to dryness by rotary evaporation. The residue was purified by column chromatography (dichloromethane:methanol=10:1) to give the target product as a pale yellow solid (80 mg, 84.0% yield). LC-MS: 357.3 [M+H]⁺.

### Step 5

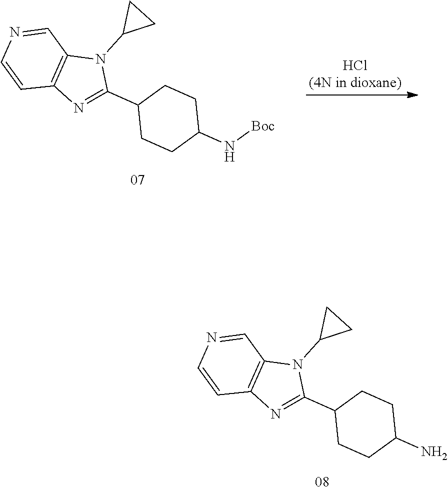 [SMILES: CC(C)(C)OC(=O)NC1CCC(c2nc3ccncc3n2C2CC2)CC1 ; NC1CCC(c2nc3ccncc3n2C2CC2)CC1]

In a 50 mL single-neck flask, 07 (40 mg, 0.112 mmol) was dissolved in dichloromethane (5 mL), and hydrochloric acid in 1,4-dioxane (0.5 mL) was added. The reaction mixture was stirred at room temperature for 1 h and could be directly used in the next step after being concentrated to dryness by rotary evaporation. LC-MS: 257.3 [M+H]⁺.

### Step 6

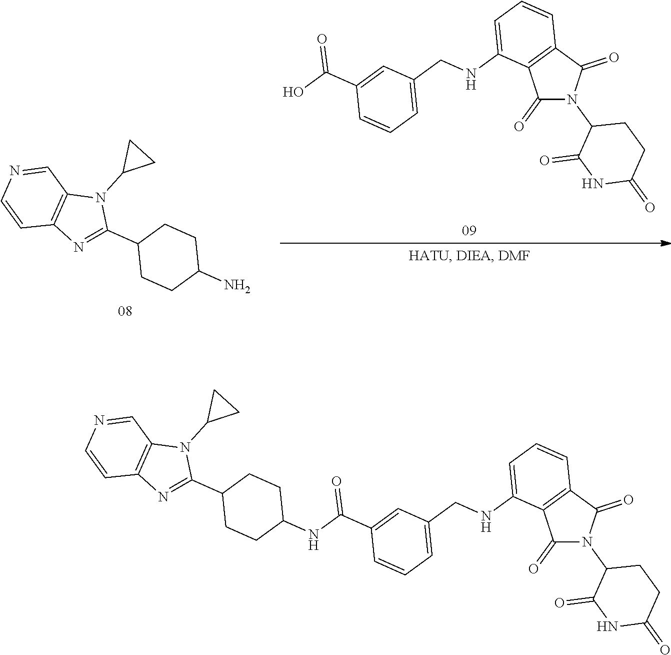 [SMILES: NC1CCC(c2nc3ccncc3n2C2CC2)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6nc7ccncc7n6C6CC6)CC5)c4)c3C2=O)C(=O)N1]

08 (27 mg, 0.105 mmol) was dissolved in N,N-dimethylformamide (5 mL) at room temperature, and 09 (42 mg, 0.103 mmol), HATU (67 mg, 0.176 mmol) and N,N-diisopropylethylamine (46 mg, 0.356 mmol) were added successively. The above mixture was stirred at room temperature for 1 h. The reaction mixture was purified by preparative high performance liquid chromatography to give a yellow solid (37.98 mg, 55.8% yield). LC-MS: 646.4 [M+H]⁺, ¹H NMR (400 MHz, DMSO-d₆) δ 11.10 (s, 1H), 8.89 (s, 1H), 8.38-8.22 (m, 2H), 7.88 (s, 1H), 7.74 (d, J=7.7 Hz, 1H), 7.56 (d, J=5.4 Hz, 1H), 7.54-7.48 (m, 2H), 7.43 (t, J=7.6 Hz, 1H), 7.29 (t, J=6.3 Hz, 1H), 7.04 (d, J=7.0 Hz, 1H), 6.96 (d, J=8.6 Hz, 1H), 5.08 (dd, J=12.9, 5.4 Hz, 1H), 4.62 (d, J=6.1 Hz, 2H), 3.89 (d, J=7.7 Hz, 1H), 3.54-3.44 (m, 1H), 3.18 (t, J=11.5 Hz, 1H), 2.96-2.83 (m, 1H), 2.58 (dd, J=16.1, 10.4 Hz, 2H), 2.06 (dd, J=30.5, 12.4 Hz, 5H), 1.76 (dd, J=24.9, 11.2 Hz, 2H), 1.56 (dd, J=24.2, 11.6 Hz, 2H), 1.26 (d, J=5.2 Hz, 2H), 1.13 (d, J=2.8 Hz, 2H).

### Example 83

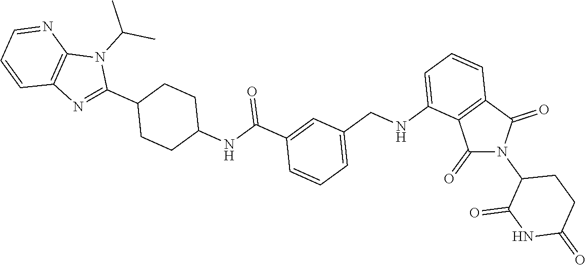 [SMILES: CC(C)n1c(C2CCC(NC(=O)c3cccc(CNc4cccc5c4C(=O)N(C4CCC(=O)NC4=O)C5=O)c3)CC2)nc2cccnc21]

### Preparation Steps

### Step 1

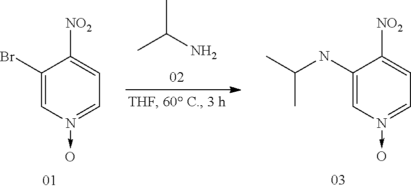

In a 100 mL single-neck flask, 01 (1 g, 4.57 mmol) was dissolved in tetrahydrofuran (10 mL), and 02 (1.35 g, 22.84 mmol) was added. The mixture was reacted at 60° C. for 3 h, quenched with water (20 mL), and extracted with ethyl acetate (20 mL×3). The organic phase was washed with saturated brine (20 mL×3) and concentrated to dryness by rotary evaporation, and the residue was purified by column chromatography (ethyl acetate:petroleum ether=1:1) to give the target product as a yellow solid (880 mg, 97.7% yield).

LC-MS: 198.1 [M+H]⁺.

### Step 2

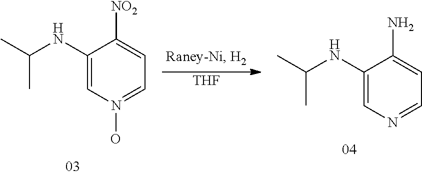

In a 100 mL single-neck flask, 03 (830 mg, 4.21 mmol) was dissolved in tetrahydrofuran (40 mL), and Raney nickel (100 mg) was added. The mixture was reacted in a hydrogen atmosphere at room temperature for 2 h and filtered, and the filtrate was concentrated to dryness by rotary evaporation to give the target product as a deep purple solid (610 mg, 90.4% yield).

LC-MS: 152.2 [M+H]⁺.

### Step 3

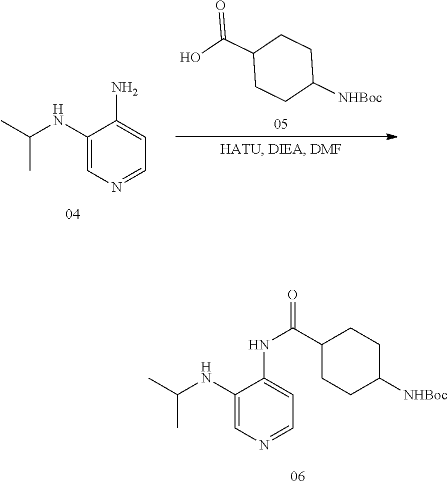 [SMILES: CC(C)Nc1cnccc1N ; CC(C)(C)OC(=O)NC1CCC(C(=O)O)CC1 ; CC(C)Nc1cnccc1NC(=O)C1CCC(NC(=O)OC(C)(C)C)CC1]

In a 100 mL single-neck flask, 04 (400 mg, 2.64 mmol) was dissolved in N,N-dimethylformamide (5 mL), and 05 (611 mg, 2.51 mmol), HATU (1.51 g, 3.97 mmol) and N,N-diisopropylethylamine (1.03 g, 7.97 mmol) were added. The mixture was reacted at room temperature for 16 h, quenched with water (15 mL), and extracted with ethyl acetate (20 mL×3). The organic phase was washed with saturated brine (20 mL×3) and concentrated to dryness by rotary evaporation, and the residue was purified by column chromatography (dichloromethane:methanol=10:1) to give the target product as a red solid (472 mg, 47.4% yield).

LC-MS: 377.2 [M+H]⁺.

### Step 4

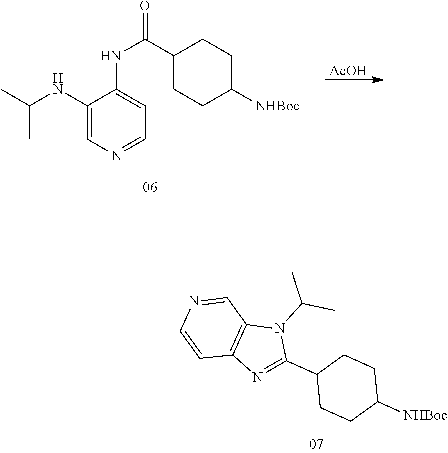 [SMILES: "AcOH" ; CC(C)Nc1cnccc1NC(=O)C1CCC(NC(=O)OC(C)(C)C)CC1 ; CC(C)n1c(C2CCC(NC(=O)OC(C)(C)C)CC2)nc2ccncc21]

In a 100 mL single-neck flask, 06 (272 mg, 0.722 mmol) was dissolved in acetic acid (3 mL), and the mixture was reacted at 70° C. for 2.5 h and concentrated to dryness by rotary evaporation. The residue was purified by column chromatography (dichloromethane:methanol=10:1) to give the target product as an orange solid (210 mg, 81.1% yield).

LC-MS: 359.2 [M+H]⁺.

### Step 5

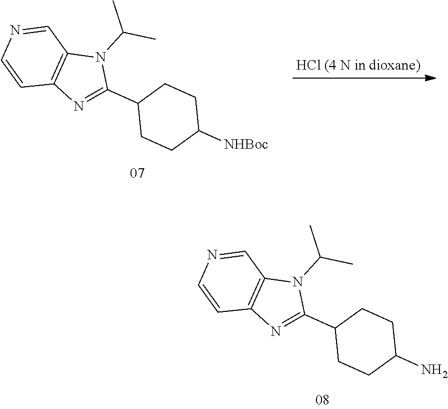 [SMILES: CC(C)n1c(C2CCC(NC(=O)OC(C)(C)C)CC2)nc2ccncc21 ; CC(C)n1c(C2CCC(N)CC2)nc2ccncc21]

In a 50 mL single-neck flask, 07 (100 mg, 0.279 mmol) was dissolved in dichloromethane (5 mL), and hydrochloric acid in 1,4-dioxane (0.5 mL) was added. The reaction mixture was stirred at room temperature for 1 h and concentrated under reduced pressure to give the target crude product (80 mg), which was directly used in the next step.

LC-MS: 259.3 [M+H]⁺.

### Step 6

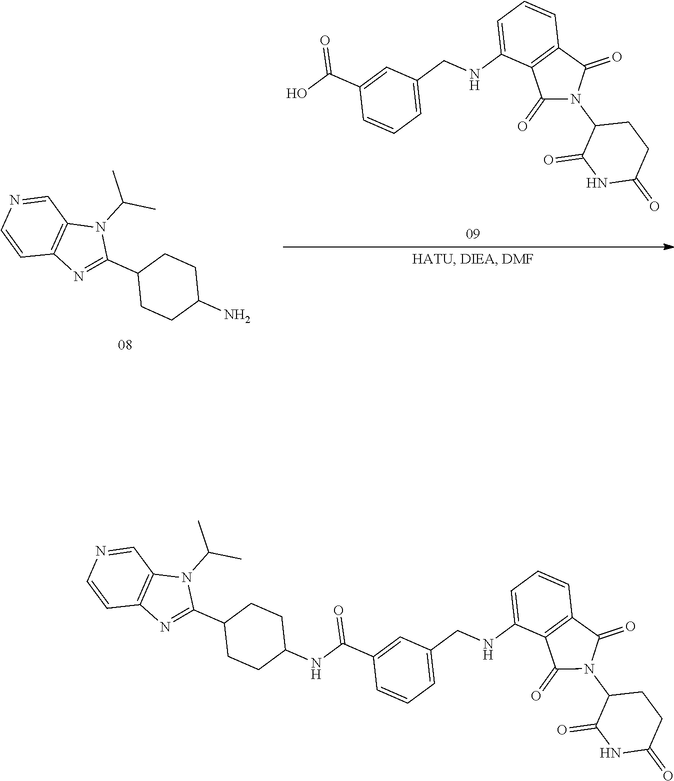 [SMILES: CC(C)n1c(C2CCC(N)CC2)nc2ccncc21 ; O=C1CCC(N2C(=O)c3cccc(NCc4cccc(C(=O)O)c4)c3C2=O)C(=O)N1 ; CC(C)n1c(C2CCC(NC(=O)c3cccc(CNc4cccc5c4C(=O)N(C4CCC(=O)NC4=O)C5=O)c3)CC2)nc2ccncc21]

08 (80 mg, 0.279 mmol) was dissolved in N,N-dimethylformamide (1 mL) at room temperature, and 09 (114 mg, 0.279 mmol), HATU (159 mg, 0.418 mmol) and N,N-diisopropylethylamine (108 mg, 0.837 mmol) were added successively. The above mixture was stirred at room temperature for 1 h. The reaction mixture was purified by preparative high performance liquid chromatography (TFA) to give a yellowish-green solid (59.89 mg, 33.1% yield).

LC-MS: 648.2 [M+H]⁺.

¹H NMR (400 MHz, DMSO-d₆) δ 11.10 (s, 0.69H), 10.97 (s, 0.26H), 9.60 (s, 1H), 8.57 (d, J=6.4 Hz, 1H), 8.45-8.32 (m, 1H), 8.18 (dd, J=15.5, 6.4 Hz, 1H), 7.86 (s, 1H), 7.78-7.72 (m, 1H), 7.58 (d, J=7.6 Hz, 1H), 7.54-7.49 (m, 1H), 7.43 (t, J=6.6 Hz, 1H), 7.29 (s, 1H), 7.14 (dd, J=30.3, 7.8 Hz, 1H), 7.06-6.93 (m, 1H), 5.16-5.02 (m, 2H), 4.78 (s, 1H), 4.62 (s, 1H), 3.31-3.18 (m, 1H), 2.96-2.79 (m, 1H), 2.67-2.54 (m, 2H), 2.24-1.95 (m, 6H), 1.83 (d, J=10.8 Hz, 3H), 1.65 (d, J=6.7 Hz, 7H).

### Example 84

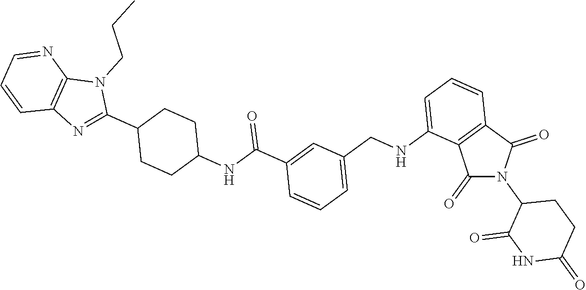 [SMILES: CCCn1c(C2CCC(NC(=O)c3cccc(CNc4cccc5c4C(=O)N(C4CCC(=O)NC4=O)C5=O)c3)CC2)nc2cccnc21]

### Preparation Steps

### Step 1

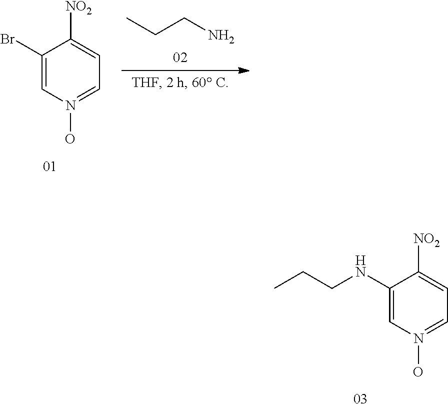

To a 100 mL single-neck flask were added 01 (1 g, 4.57 mmol), 02 (1.35 g, 22.84 mmol) and tetrahydrofuran (20 mL), and the mixture was reacted at 60° C. for 2 h, quenched with water (20 mL), and extracted with ethyl acetate (20 mL×2). The organic phase was washed with saturated brine (20 mL×3) and concentrated to dryness by rotary evaporation, and the residue was purified by column chromatography (ethyl acetate:petroleum ether=1:1) to give the target product as a yellow solid (800 mg, 88.8% yield). LC-MS: 198.2 [M+H]⁺.

### Step 2

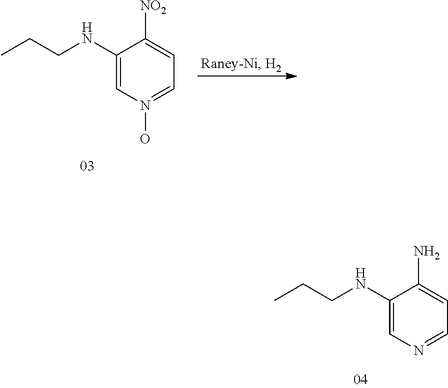

To a 100 mL single-neck flask were added 03 (800 mg, 4.06 mmol), Raney nickel (300 mg) and tetrahydrofuran (40 mL), and the mixture was reacted in a hydrogen atmosphere at room temperature for 2 h and filtered. The filtrate was concentrated to dryness by rotary evaporation to give the target product as a red solid (580 mg, 94.5% yield). LC-MS: 152.2 [M+H]⁺.

### Step 3

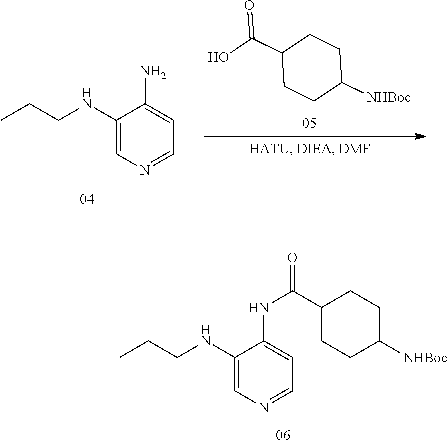 [SMILES: CCCNc1cnccc1N ; CC(C)(C)OC(=O)NC1CCC(C(=O)O)CC1 ; CCCNc1cnccc1NC(=O)C1CCC(NC(=O)OC(C)(C)C)CC1]

To a 100 mL single-neck flask were added 04 (400 mg, 2.65 mmol), 05 (611 mg, 2.51 mmol), HATU (1.51 g, 3.97 mmol), DIEA (1.03 g, 7.97 mmol) and DMF (20 mL), and the mixture was reacted at room temperature for 2 h, quenched with water (20 mL), and extracted with ethyl acetate (20 mL×2). The organic phase was washed with saturated brine (20 mL×3) and concentrated to dryness by rotary evaporation, and the residue was purified by column chromatography (dichloromethane:methanol=20:1) to give the target product as a yellow solid (600 mg, 60.2% yield). LC-MS: 377.3 [M+H]⁺.

### Step 4

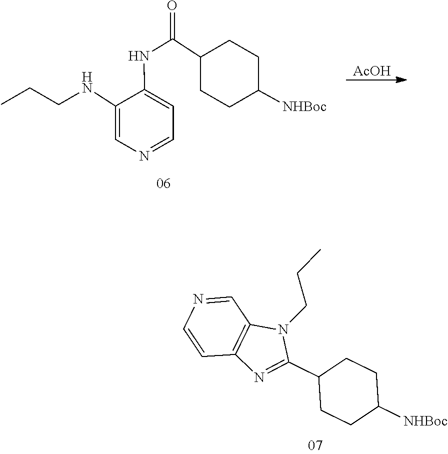 [SMILES: "AcOH" ; CCCNc1cnccc1NC(=O)C1CCC(NC(=O)OC(C)(C)C)CC1 ; CCCn1c(C2CCC(NC(=O)OC(C)(C)C)CC2)nc2ccncc21]

In a 100 mL single-neck round-bottom flask, 06 (300 mg, 0.797 mmol) was dissolved in acetic acid (5 mL), and the mixture was reacted at 70° C. for 2.5 h and concentrated to dryness by rotary evaporation. The residue was purified by column chromatography (dichloromethane:methanol=10:1) to give the target product as a pale yellow solid (280 mg, 98.0% yield). LC-MS: 359.2 [M+H]⁺.

### Step 5

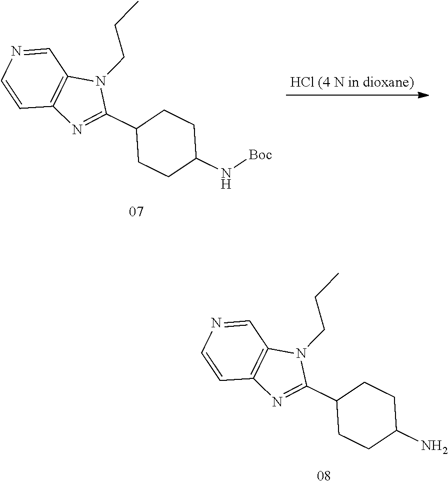 [SMILES: CCCn1c(C2CCC(NC(=O)OC(C)(C)C)CC2)nc2ccncc21 ; CCCn1c(C2CCC(N)CC2)nc2ccncc21]

In a 50 mL single-neck flask, 07 (65 mg, 0.181 mmol) was dissolved in dichloromethane (5 mL), and hydrochloric acid in 1,4-dioxane (0.5 mL) was added, and the reaction mixture was stirred at room temperature for 1 h and concentrated to dryness by rotary evaporation to give the target compound (40 mg, crude), which could be directly used in the next step without purification. LC-MS: 259.2 [M+H]⁺.

### Step 6

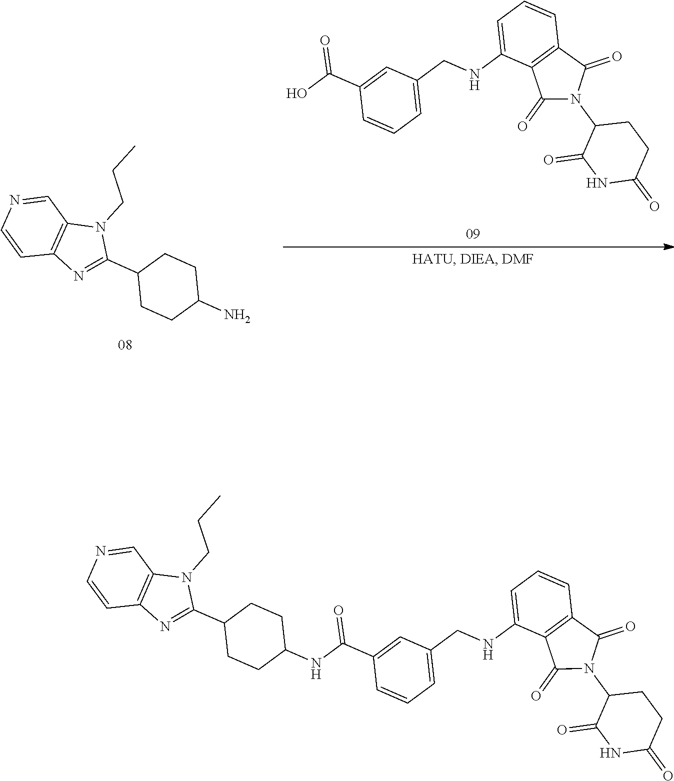 [SMILES: CCCn1c(C2CCC(N)CC2)nc2ccncc21 ; O=C1CCC(N2C(=O)c3cccc(NCc4cccc(C(=O)O)c4)c3C2=O)C(=O)N1 ; CCCn1c(C2CCC(NC(=O)c3cccc(CNc4cccc5c4C(=O)N(C4CCC(=O)NC4=O)C5=O)c3)CC2)nc2ccncc21]

08 (40 mg, 0.155 mmol) was dissolved in N,N-dimethylformamide (5 mL) at room temperature, and 09 (63 mg, 0.155 mmol), HATU (88 mg, 0.231 mmol) and N,N-diisopropylethylamine (100 mg, 0.774 mmol) were added successively. The above mixture was stirred at room temperature for 1 h. The reaction mixture was purified by preparative high performance liquid chromatography to give a yellowish-green solid (60.65 mg, 60.5% yield). LC-MS: 648.3 [M+H]⁺, ¹H NMR (400 MHz, DMSO-d₆) δ 11.10 (s, 1H), 9.54 (s, 1H), 8.58 (d, J=6.4 Hz, 1H), 8.34 (d, J=7.6 Hz, 1H), 8.19 (d, J=6.4 Hz, 1H), 7.86 (s, 1H), 7.74 (d, J=8.3 Hz, 1H), 7.51 (d, J=7.3 Hz, 2H), 7.43 (t, J=7.6 Hz, 1H), 7.29 (s, 1H), 7.04 (d, J=7.1 Hz, 1H), 6.96 (d, J=8.5 Hz, 1H), 5.08 (dd, J=12.8, 5.5 Hz, 1H), 4.62 (s, 2H), 4.50-4.46 (m, 2H), 3.87 (s, 1H), 3.16 (s, 1H), 2.90 (t, J=12.7 Hz, 1H), 2.67-2.55 (m, 2H), 2.03 (d, J=11.9 Hz, 6H), 1.83 (dd, J=14.7, 7.9 Hz, 4H), 1.66-1.56 (m, 2H), 0.94 (t, J=7.3 Hz, 3H).

### Example 85

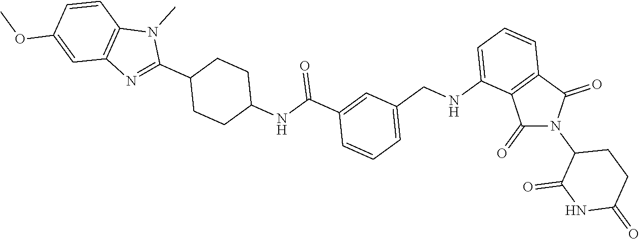 [SMILES: COc1ccc2c(c1)nc(C1CCC(NC(=O)c3cccc(CNc4cccc5c4C(=O)N(C4CCC(=O)NC4=O)C5=O)c3)CC1)n2C]

### Preparation Steps

### Step 1

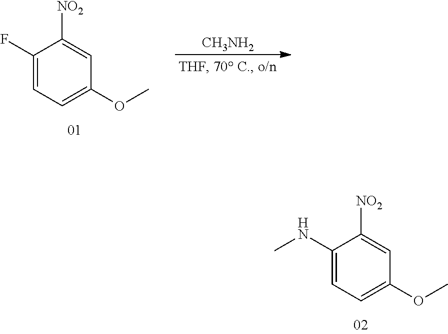

To a 100 mL single-neck flask were added 01 (1 g, 5.84 mmol), 02 (3.36 g, 29.21 mmol) and tetrahydrofuran (20 mL), and the mixture was reacted at 70° C. for 12 h, quenched with water (20 mL), and extracted with ethyl acetate (20 mL×2). The organic phase was washed with saturated brine (20 mL×3) and concentrated to dryness by rotary evaporation, and the residue was purified by column chromatography (ethyl acetate:petroleum ether=1:1) to give the target product as a yellow solid (800 mg, 75.1% yield). LC-MS: 183.1 [M+H]⁺.

### Step 2

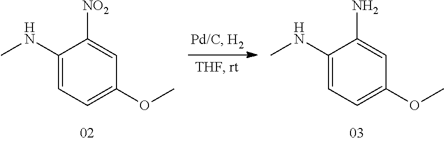

To a 100 mL single-neck flask were added 02 (800 mg, 4.39 mmol), Raney nickel (300 mg) and tetrahydrofuran (40 mL), and the mixture was reacted in a hydrogen atmosphere at room temperature for 2 h and filtered. The filtrate was concentrated to dryness by rotary evaporation to give the target product as a red solid (400 mg, 59.8% yield). LC-MS: 153.2 [M+H]⁺.

### Step 3

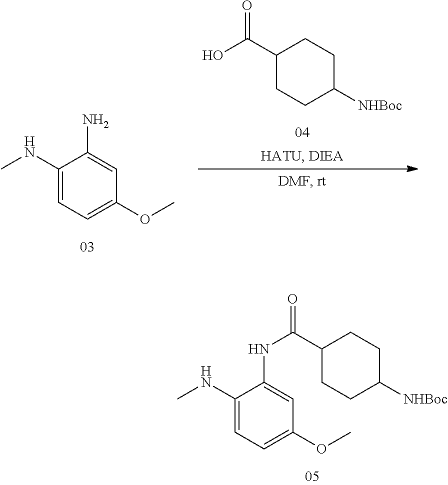

To a 100 mL single-neck flask were added 03 (200 mg, 1.31 mmol), 04 (310 mg, 1.27 mmol), HATU (749 mg, 1.97 mmol), DIEA (509 mg, 3.94 mmol) and DMF (10 mL), and the mixture was reacted at room temperature for 2 h, quenched with water (20 mL), and extracted with ethyl acetate (20 mL×2). The organic phase was washed with saturated brine (20 mL×3) and concentrated to dryness by rotary evaporation, and the residue was purified by column chromatography (dichloromethane:methanol=20:1) to give the target product as a yellow solid (100 mg, 20.2% yield). LC-MS: 378.2 [M+H]⁺.

### Step 4

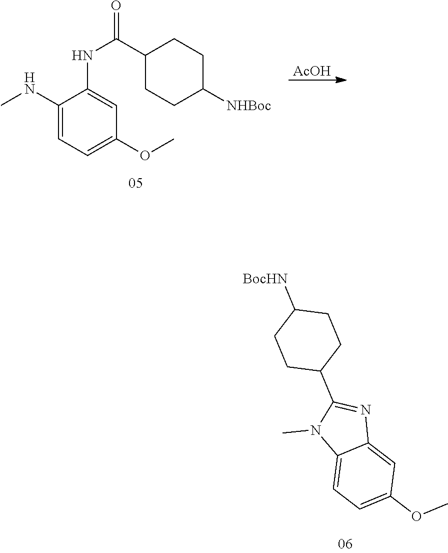 [SMILES: "AcOH" ; CNc1ccc(OC)cc1NC(=O)C1CCC(NC(=O)OC(C)(C)C)CC1 ; COc1ccc2c(c1)nc(C1CCC(NC(=O)OC(C)(C)C)CC1)n2C]

In a 100 mL single-neck round-bottom flask, 05 (100 mg, 0.265 mmol) was dissolved in acetic acid (2 mL), and the mixture was reacted at 70° C. for 2.5 h and concentrated to dryness by rotary evaporation. The residue was purified by column chromatography (dichloromethane:methanol=10:1) to give the target product as a pale yellow solid (40 mg, 42.0% yield). LC-MS: 360.1 [M+H]⁺.

### Step 5

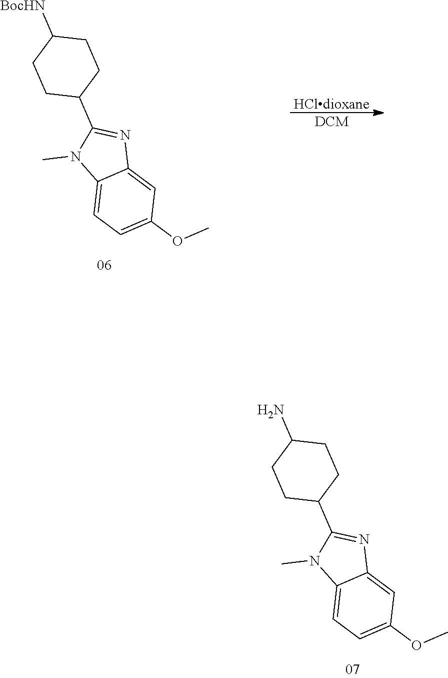

In a 50 mL single-neck flask, 06 (40 mg, 0.111 mmol) was dissolved in dichloromethane (5 mL), and hydrochloric acid in 1,4-dioxane (0.5 mL) was added. The reaction mixture was stirred at room temperature for 1 h and could be directly used in the next step after being concentrated to dryness by rotary evaporation. LC-MS: 260.4 [M+H]⁺.

### Step 6

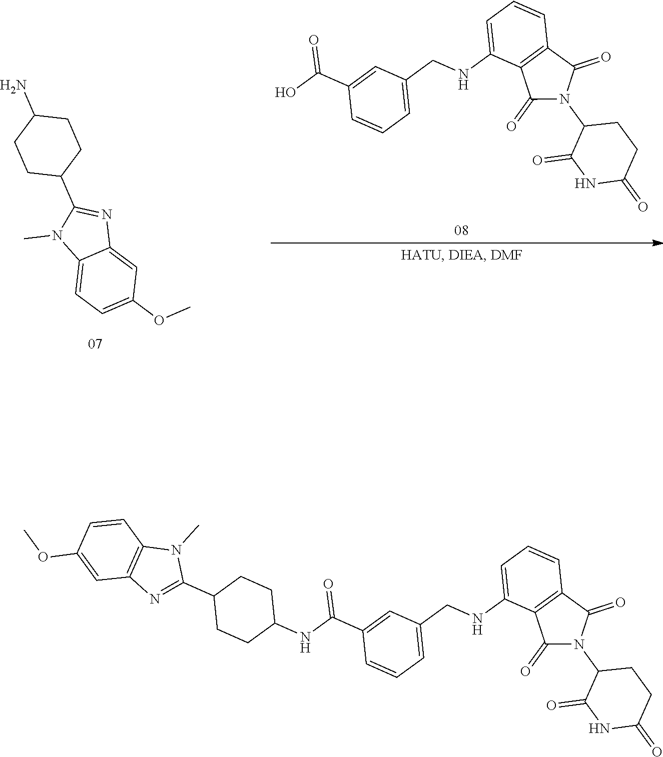 [SMILES: COc1ccc2c(c1)nc(C1CCC(N)CC1)n2C ; O=C1CCC(N2C(=O)c3cccc(NCc4cccc(C(=O)O)c4)c3C2=O)C(=O)N1 ; COc1ccc2c(c1)nc(C1CCC(NC(=O)c3cccc(CNc4cccc5c4C(=O)N(C4CCC(=O)NC4=O)C5=O)c3)CC1)n2C]

07 (18 mg, 0.0694 mmol) was dissolved in N,N-dimethylformamide (1 mL) at room temperature, and 08 (28 mg, 0.0687 mmol), HATU (40 mg, 0.105 mmol) and N,N-diisopropylethylamine (27 mg, 0.209 mmol) were added successively. The above mixture was stirred at room temperature for 1 h. The reaction mixture was purified by preparative high performance liquid chromatography to give a yellowish-green solid (25.59 mg, 56.8% yield). LC-MS: 649.3 [M+H]⁺, ¹H NMR (400 MHz, DMSO-d6) δ 11.10 (s, 1H), 8.31 (d, J=7.5 Hz, 1H), 7.87 (s, 1H), 7.74 (d, J=7.9 Hz, 1H), 7.56-7.47 (m, 2H), 7.42 (t, J=7.7 Hz, 1H), 7.36 (d, J=8.7 Hz, 1H), 7.29 (s, 1H), 7.11 (d, J=2.3 Hz, 1H), 7.04 (d, J=7.0 Hz, 1H), 6.96 (d, J=8.4 Hz, 1H), 6.81 (dd, J=8.8, 2.4 Hz, 1H), 5.08 (dd, J=12.7, 5.3 Hz, 1H), 4.62 (d, J=5.9 Hz, 2H), 3.75 (d, J=7.0 Hz, 7H), 2.91 (s, 2H), 1.99 (s, 6H), 1.73 (d, J=13.9 Hz, 3H), 1.56 (d, J=10.5 Hz, 2H).

### Example 86

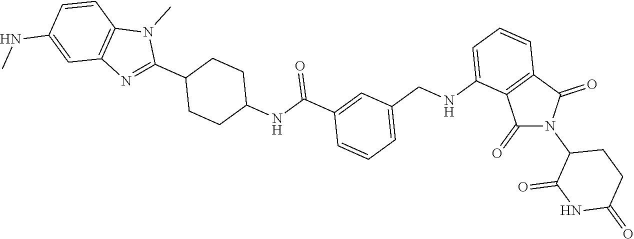 [SMILES: CNc1ccc2c(c1)nc(C1CCC(NC(=O)c3cccc(CNc4cccc5c4C(=O)N(C4CCC(=O)NC4=O)C5=O)c3)CC1)n2C]

### Preparation Steps

### Step 1

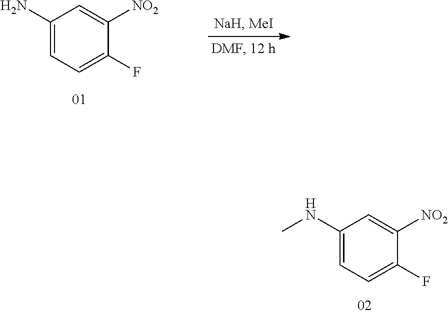

Sodium hydride (307 mg, 7.67 mmol, 60%) was added to a 100 mL three-necked flask, purged, and cooled to 0° C. 01 (1 g, 6.4 mmol) was dissolved in DMF, and the resulting solution was added to the reaction system. After the reaction system was reacted at 0° C. for 0.5 h, iodomethane (1.36 g, 9.58 mmol) was added. The reaction system was reacted at 25° C. for 12 h, diluted with water (50 mL), and extracted with ethyl acetate (30 mL×3). The organic phase was washed with saturated brine (30 mL×2), dried over anhydrous sodium sulfate, and concentrated to dryness by rotary evaporation, and the residue was purified by column chromatography (petroleum ether:ethyl acetate=3:1) to give the target product as a red solid (300 mg, 28% yield). LC-MS: 171.1 [M+H]⁺.

### Step 2

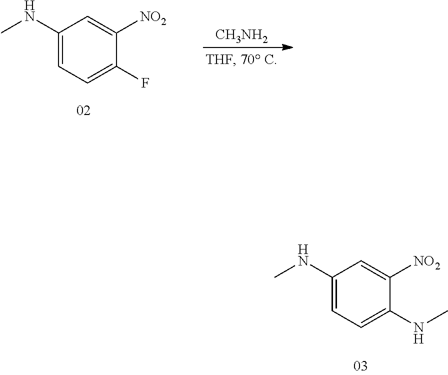

To a 100 mL single-neck flask were added 02 (0.3 g, 1.76 mmol), a solution of methylamine in alcohol (1.01 g, 8.8 mmol) and tetrahydrofuran (20 mL), and the mixture was reacted at 70° C. for 36 h, quenched with water (20 mL), and extracted with ethyl acetate (20 mL×2). The organic phase was washed with saturated brine (20 mL×3) and concentrated to dryness by rotary evaporation, and the residue was purified by column chromatography (ethyl acetate:petroleum ether=1:1) to give the target product as a black solid (230 mg, 72% yield). LC-MS: 182.2 [M+H]⁺.

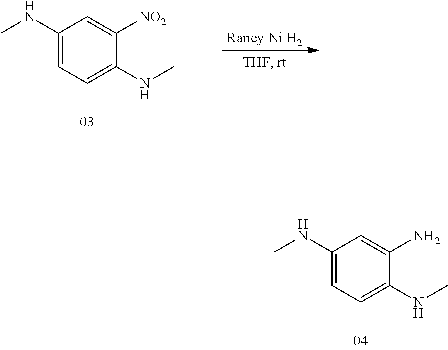

To a 100 mL single-neck flask were added 03 (230 mg, 1.27 mmol), Raney nickel (200 mg) and tetrahydrofuran (20 mL), and the mixture was reacted in a hydrogen atmosphere at room temperature for 2 h and filtered. The filtrate was concentrated to dryness by rotary evaporation to give the target product as a red solid (150 mg, 78% yield). LC-MS: 152.2 [M+H]⁺.

### Step 4

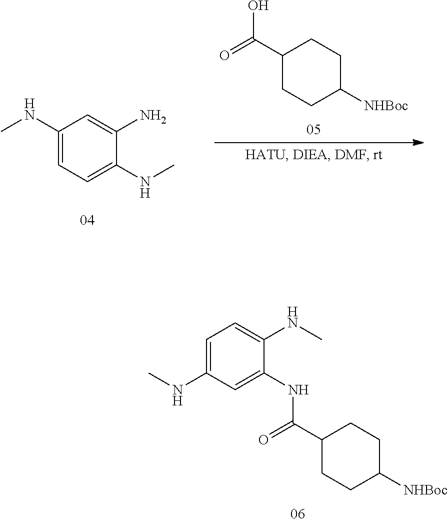

To a 100 mL single-neck flask were added 04 (150 mg, 0.992 mmol), 05 (180 mg, 0.74 mmol), HATU (458 mg, 1.20 mmol), DIEA (359 mg, 2.78 mmol) and DMF (10 mL), and the mixture was reacted at room temperature for 2 h, quenched with water (20 mL), and extracted with ethyl acetate (20 mL×2). The organic phase was washed with saturated brine (20 mL×3) and concentrated to dryness by rotary evaporation, and the residue was purified by column chromatography (dichloromethane:methanol=10:1) to give the target product as a white solid (100 mg, 36% yield). LC-MS: 377.3 [M+H]⁺.

### Step 5

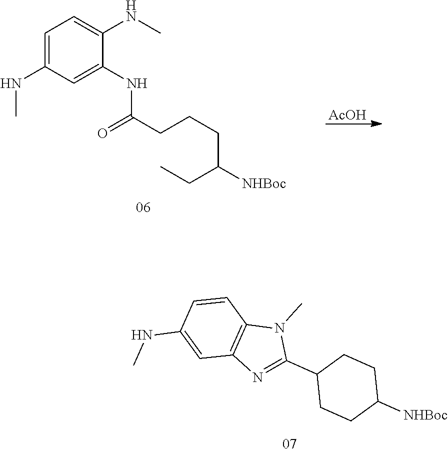 [SMILES: "AcOH" ; CCC(CCCC(=O)Nc1cc(NC)ccc1NC)NC(=O)OC(C)(C)C ; CNc1ccc2c(c1)nc(C1CCC(NC(=O)OC(C)(C)C)CC1)n2C]

In a 100 mL single-neck round-bottom flask, 06 (100 mg, 0.267 mmol) was dissolved in acetic acid (1 mL), and the mixture was reacted at 70° C. for 4 h concentrated to dryness by rotary evaporation, diluted with ethyl acetate (20 mL×2), adjusted to pH 8-9 with saturated aqueous sodium bicarbonate solution (20 mL×3), washed with saturated brine (20 mL×2), dried, concentrated, and purified by column chromatography (dichloromethane:methanol=10:1) to give the target product 07 as a pale yellow solid (18 mg, 19% yield). LC-MS: 359.2 [M+H]⁺.

### Step 6

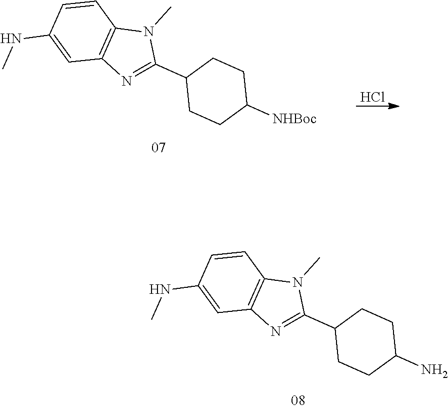 [SMILES: CNc1ccc2c(c1)nc(C1CCC(NC(=O)OC(C)(C)C)CC1)n2C ; Cl ; CNc1ccc2c(c1)nc(C1CCC(N)CC1)n2C]

In a 50 mL single-neck flask, 07 (18 mg, 0.0502 mmol) was dissolved in dichloromethane (2 mL), and hydrochloric acid in 1,4-dioxane (0.5 mL) was added. The reaction mixture was stirred at room temperature for 1 h and could be directly used in the next step after being concentrated to dryness by rotary evaporation (18 mg, crude). LC-MS: 259.4 [M+H]⁺.

### Step 7

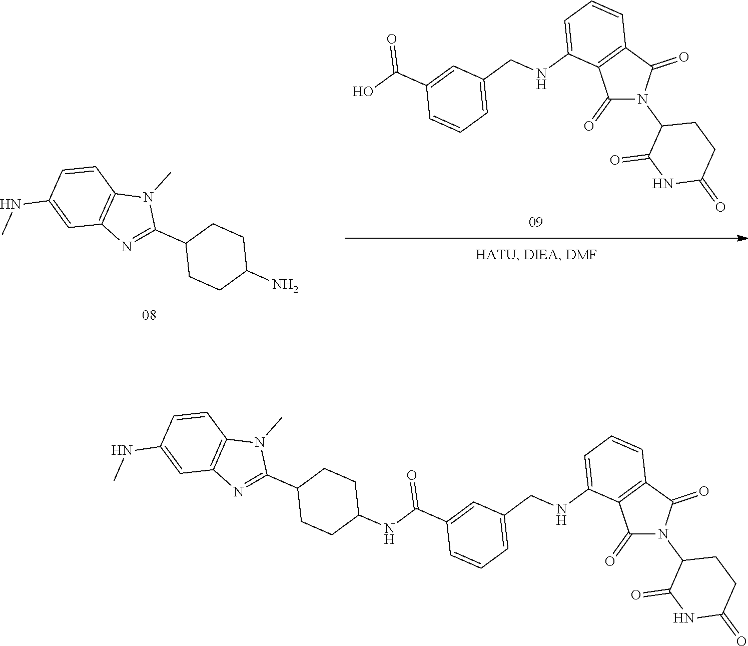 [SMILES: CNc1ccc2c(c1)nc(C1CCC(N)CC1)n2C ; O=C1CCC(N2C(=O)c3cccc(NCc4cccc(C(=O)O)c4)c3C2=O)C(=O)N1 ; CNc1ccc2c(c1)nc(C1CCC(NC(=O)c3cccc(CNc4cccc5c4C(=O)N(C4CCC(=O)NC4=O)C5=O)c3)CC1)n2C]

08 (18 mg, 0.0502 mmol) was dissolved in N,N-dimethylformamide (1 mL) at room temperature, and 09 (18 mg, 0.044 mmol), HATU (20 mg, 0.053 mmol) and N,N-diisopropylethylamine (30 mg, 0.232 mmol) were added successively. The above mixture was stirred at room temperature for 1 h. The reaction mixture was purified by preparative high performance liquid chromatography to give a yellowish-green solid (9.55 mg, 33% yield over two steps). LC-MS: 648.3 [M+H]⁺, ¹H NMR (400 MHz, DMSO-d₆) δ 11.10 (s, 1H), 8.30 (s, 2H), 7.87 (s, 1H), 7.74 (d, J=7.7 Hz, 1H), 7.56-7.48 (m, 2H), 7.42 (t, J=7.6 Hz, 1H), 7.29 (t, J=6.2 Hz, 1H), 7.17 (d, J=8.6 Hz, 1H), 7.03 (d, J=7.1 Hz, 1H), 6.96 (d, J=8.7 Hz, 1H), 6.62 (d, J=1.7 Hz, 1H), 6.54 (dd, J=8.6, 2.0 Hz, 1H), 5.08 (dd, J=12.8, 5.4 Hz, 1H), 4.62 (d, J=6.1 Hz, 2H), 3.87-3.80 (m, 1H), 3.67 (s, 3H), 2.91 (dd, J=23.5, 7.5 Hz, 2H), 2.68 (s, 3H), 2.62-2.56 (m, 2H), 2.08-2.02 (m, 1H), 1.98 (d, J=9.3 Hz, 4H), 1.71 (dd, J=23.1, 10.3 Hz, 2H), 1.54 (dd, J=22.5, 11.7 Hz, 2H).

### Example 87

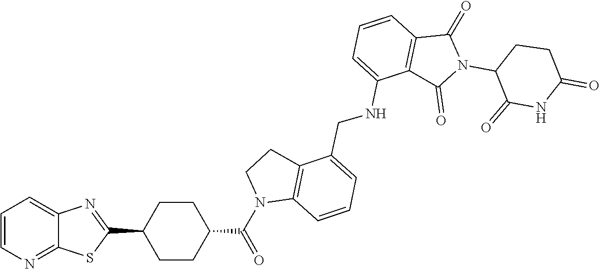 [SMILES: O=C1CCC(N2C(=O)c3cccc(NCc4cccc5c4CCN5C(=O)[C@H]4CC[C@H](c5nc6cccnc6s5)CC4)c3C2=O)C(=O)N1]

### Preparation Steps

### Step 1

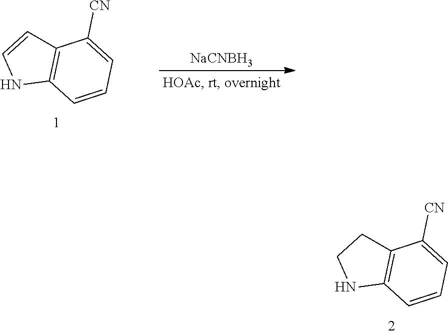 [SMILES: N#Cc1cccc2[nH]ccc12 ; "NaCNBH3" ; N#Cc1cccc2c1CCN2]

Compound 1 (2 g, 14.08 mmol) was dissolved in acetic acid solution (10 mL), and sodium cyanoborohydride (1.7 g, 28.16 mmol) was added. The mixture was stirred overnight at room temperature. After the reaction was completed, water was added to the reaction mixture, and the aqueous phase was extracted three times with ethyl acetate. The organic phases were combined, dried over anhydrous sodium sulfate, and filtered, and the filtrate was concentrated to give a crude product, compound 2 (1.8 g, 88.67% yield, brown oil). LC-MS: 145.1 [M+H]⁺.

### Step 2

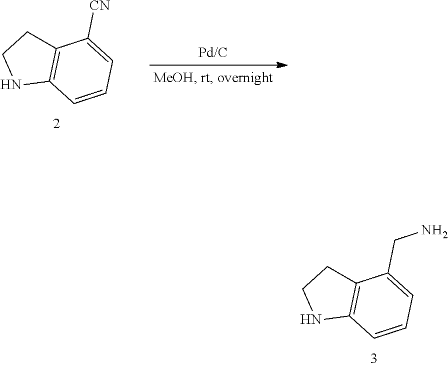 [SMILES: N#Cc1cccc2c1CCN2 ; NCc1cccc2c1CCN2]

Compound 2 (1.8 g, 12.5 mmol) was dissolved in MeOH (10 mL) at room temperature, and Pd/C (180 mg) was added. The reaction mixture was stirred at room temperature overnight and concentrated to give a crude product, which was then purified by acidic reversed-phase chromatography to give a crude product, compound 3 (200 mg, 10.81% yield, white solid). LC-MS: 149.1 [M+H]⁺.

### Step 3

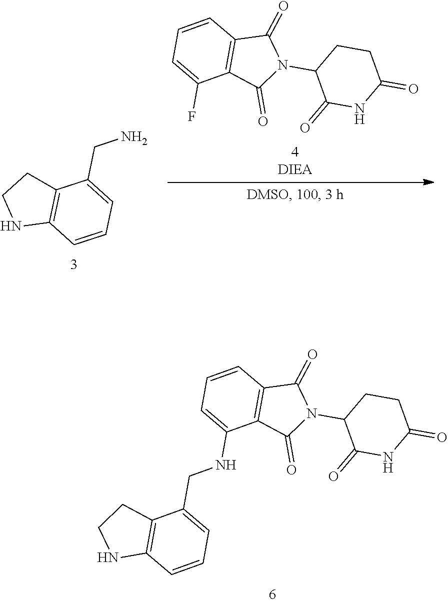 [SMILES: O=C1CCC(N2C(=O)c3cccc(F)c3C2=O)C(=O)N1 ; NCc1cccc2c1CCN2 ; O=C1CCC(N2C(=O)c3cccc(NCc4cccc5c4CCN5)c3C2=O)C(=O)N1]

Compound 3 (150 mg, 1.01 mmol) and compound 4 (279 mg, 1.01 mmol) were dissolved in DMSO solution (2 mL) at room temperature, and DIEA (394 mg, 3.03 mmol) was added. The reaction mixture was stirred at 100° C. for 3 h and concentrated to give a crude product, which was then purified by acidic reversed-phase chromatography to give compound 6 (200 mg, 48.78% yield, yellow solid). LC-MS: 405.0 [M+H]⁺.

### Step 4

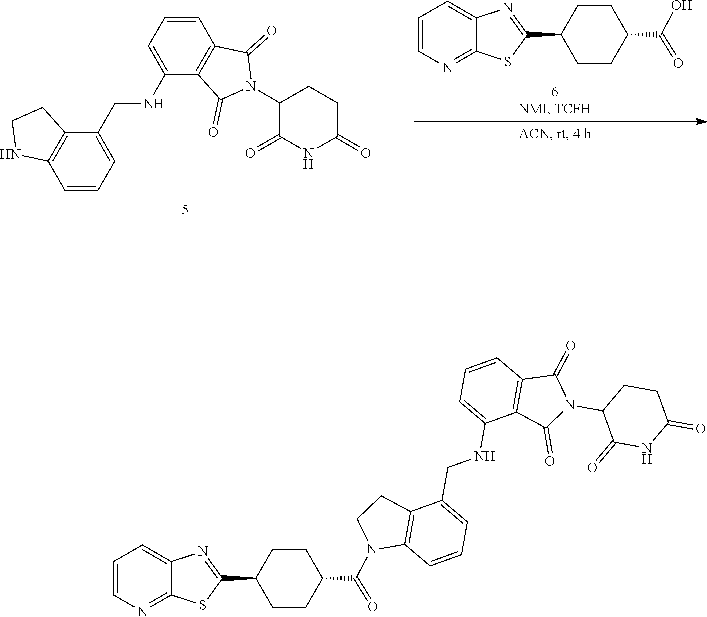 [SMILES: O=C1CCC(N2C(=O)c3cccc(NCc4cccc5c4CCN5)c3C2=O)C(=O)N1 ; O=C(O)[C@H]1CC[C@H](c2nc3cccnc3s2)CC1 ; O=C1CCC(N2C(=O)c3cccc(NCc4cccc5c4CCN5C(=O)[C@H]4CC[C@H](c5nc6cccnc6s5)CC4)c3C2=O)C(=O)N1]

Compound 5 (100 mg, 0.25 mmol) and compound 6 (84 mg, 0.33 mmol) were dissolved in acetonitrile solution (5 mL) at room temperature, and NMI (40 mg, 0.50 mmol) and N,N,N,N-tetramethylchloroformamidinium hexafluorophosphate (90 mg, 0.33 mmol) were added. The reaction mixture was stirred at room temperature for 1 h and concentrated to give a crude product, which was then purified by acidic reversed-phase chromatography to give the target product (9.96 mg, 6.21% yield, yellow solid). LC-MS: 649.1 [M+H]⁺. 1H NMR (400 MHz, DMSO-d₆) δ 11.10 (s, 1H), 8.62-8.56 (m, 1H), 8.38-8.30 (m, 1H), 8.04 (d, J=7.8 Hz, 1H), 7.60-7.44 (m, 2H), 7.13 (t, J=6.8 Hz, 2H), 7.04 (d, J=7.2 Hz, 1H), 6.94 (d, J=8.4 Hz, 2H), 5.12-5.02 (m, 1H), 4.52 (d, J=6.0 Hz, 2H), 4.27 (t, J=8.4 Hz, 2H), 3.21 (t, J=8.4 Hz, 3H), 2.97-2.84 (m, 1H), 2.70 (d, J=20.0 Hz, 1H), 2.64-2.53 (m, 2H), 2.25 (d, J=12.2 Hz, 2H), 2.10-1.96 (m, 3H), 1.80-1.62 (m, 4H).

### Example 88

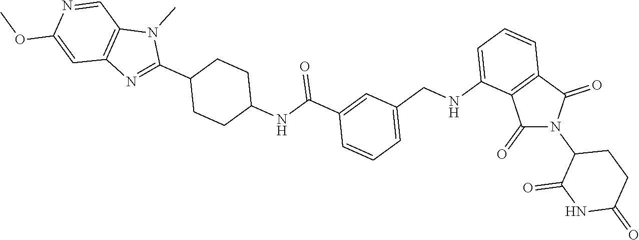 [SMILES: COc1cc2nc(C3CCC(NC(=O)c4cccc(CNc5cccc6c5C(=O)N(C5CCC(=O)NC5=O)C6=O)c4)CC3)n(C)c2cn1]

### Preparation Steps

### Step 1

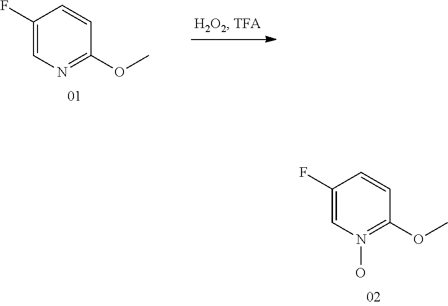

01 (10 g, 78.7 mmol) was dissolved in trifluoroacetic acid (80 mL) at room temperature, and hydrogen peroxide (30%, 39.5 mL) was added slowly with stirring at room temperature. The reaction mixture was stirred in an argon atmosphere at 70° C. for 48 h and concentrated to dryness by rotary evaporation, and the residue was purified by column chromatography (methanol:dichloromethane=1:10) to give a yellow solid (4.7 g, 42% yield). LC-MS: 144.3 [M+H]⁺.

### Step 2

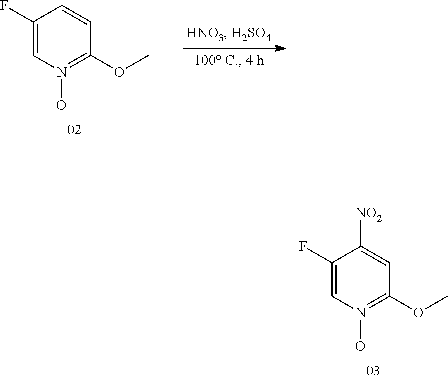

02 (4.7 g, 32.867 mmol) was dissolved in sulfuric acid (50 mL) at room temperature, and nitric acid (25 mL) was added slowly with stirring at room temperature. The reaction mixture was stirred in an argon atmosphere at 100° C. for 4 h and basified with anhydrous sodium bicarbonate. Water (100 mL) was added, followed by extraction with ethyl acetate (100 mL×3). The organic phase was washed with saturated brine (100 mL), dried over anhydrous sodium sulfate, and concentrated to dryness by rotary evaporation, and the residue was purified by column chromatography (ethyl acetate:petroleum ether=1:2) to give a yellow solid (1.236 g, 20% yield).

LC-MS: 189.2 [M+H]⁺.

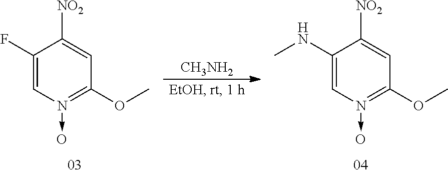

03 (1.236 g, 6.57 mmol) was dissolved in ethanol solution (40 mL) at room temperature, and a solution of methylamine in ethanol (20 mL) was added slowly with stirring at room temperature. The reaction mixture was stirred at room temperature for 1 h and concentrated to dryness by rotary evaporation to give a red solid (1.667 g, 99% yield). LC-MS: 200.1 [M+H]⁺.

### Step 4

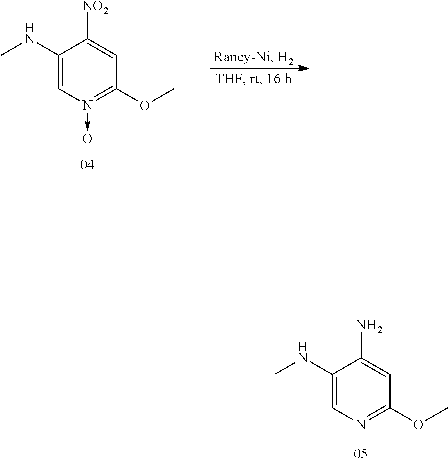

04 (800 mg, 4.02 mmol) was dissolved in THF (30 mL) at room temperature, and Raney-Ni was added at room temperature. The reaction mixture was stirred in a hydrogen atmosphere at room temperature for 4 h and filtered, and the filtrate was concentrated to dryness by rotary evaporation to give a reddish-brown solid (653 mg, 99% yield). LC-MS: 154.1 [M+H]⁺.

### Step 5

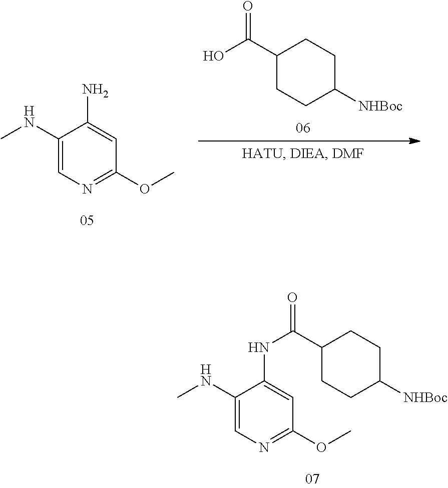

05 (300 mg, 1.958 mmol), 06 (428 mg, 1.76 mmol) and N,N-diisopropylethylamine (759 mg, 5.87 mmol) were dissolved in N,N-dimethylformamide (15 mL) at room temperature, and (7-azabenzotriazole)-tetramethyluronium hexafluorophosphate (1116 mg, 2.937 mmol) was added with stirring at room temperature. The reaction mixture was stirred at room temperature for 2 h, quenched with water (30 mL), and extracted with ethyl acetate (20 mL×3). The organic phase was washed with saturated brine (30 mL), dried over anhydrous sodium sulfate, and concentrated to dryness by rotary evaporation, and the residue was purified by column chromatography (methanol:dichloromethane=1:10) to give a brown oily liquid (584 mg, 78.9% yield). LC-MS: 379.4 [M+H]⁺.

### Step 6

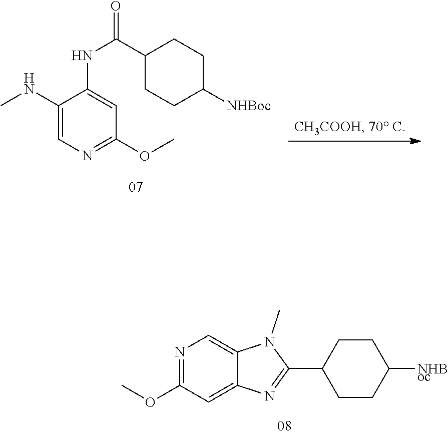 [SMILES: CNc1cnc(OC)cc1NC(=O)C1CCC(NC(=O)OC(C)(C)C)CC1 ; COc1cc2nc(C3CCC(NC(=O)OC(C)(C)C)CC3)n(C)c2cn1]

07 (534 mg, 1.41 mmol) was dissolved in acetic acid (10 mL) at room temperature, and the reaction mixture was reacted at 70° C. for 2 h and concentrated to dryness by rotary evaporation. The residue was purified through a C18 reversed-phase column to give a yellow solid (282 mg, 55.6% yield). LC-MS: 361.4 [M+H]⁺.

### Step 7

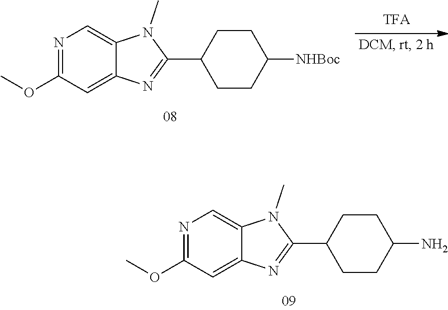 [SMILES: COc1cc2nc(C3CCC(NC(=O)OC(C)(C)C)CC3)n(C)c2cn1 ; COc1cc2nc(C3CCC(N)CC3)n(C)c2cn1]

08 (50 mg, 0.139 mmol) was dissolved in dichloromethane (2 mL) at room temperature, and trifluoroacetic acid (0.4 mL) was added with stirring at room temperature. The reaction mixture was reacted at room temperature for 12 h and concentrated to dryness by rotary evaporation to give a brown oily liquid (50 mg, 99% yield). LC-MS: 261.2 [M+H]⁺.

### Step 8

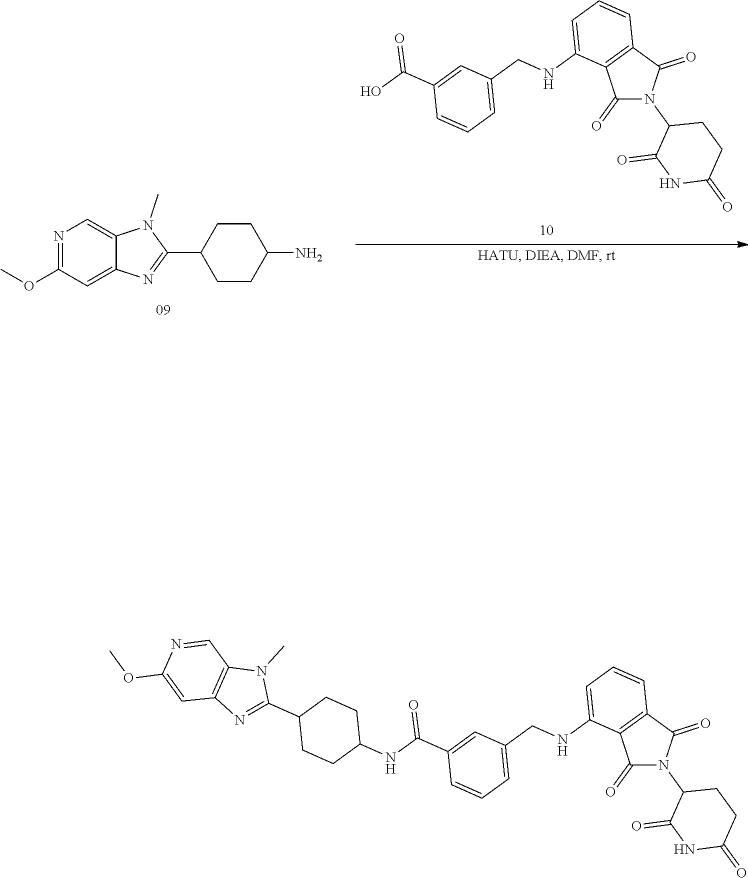 [SMILES: COc1cc2nc(C3CCC(N)CC3)n(C)c2cn1 ; O=C1CCC(N2C(=O)c3cccc(NCc4cccc(C(=O)O)c4)c3C2=O)C(=O)N1 ; COc1cc2nc(C3CCC(NC(=O)c4cccc(CNc5cccc6c5C(=O)N(C5CCC(=O)NC5=O)C6=O)c4)CC3)n(C)c2cn1]

09 (50 mg, 0.192 mmol), 10 (70 mg, 0.173 mmol) and N,N-diisopropylethylamine (74 mg, 0.576 mmol) were dissolved in DMF (1.5 mL) at room temperature, and (7-azabenzotriazole)-tetramethyluronium hexafluorophosphate (109 mg, 0.289 mmol) was added with stirring at room temperature. The reaction mixture was stirred at room temperature for 12 h and purified by preparative chromatography to give a green solid (21.07 mg, 16.9% yield). LC-MS: 650.3 [M+H]⁺.

1H NMR (400 MHz, DMSO-d₆) δ 11.11 (s, 1H), 8.69 (s, 1H), 8.36 (d, J=7.5 Hz, 1H), 7.86 (s, 1H), 7.74 (d, J=7.8 Hz, 1H), 7.55-7.48 (m, 2H), 7.43 (t, J=7.6 Hz, 1H), 7.30 (s, 1H), 7.12-7.02 (m, 2H), 6.95 (d, J=8.6 Hz, 1H), 5.08 (dd, J=12.8, 5.4 Hz, 1H), 4.62 (d, J=5.9 Hz, 2H), 3.93 (d, J=7.3 Hz, 6H), 3.88-3.82 (m, 1H), 3.13 (s, 1H), 2.89 (dd, J=21.7, 10.2 Hz, 1H), 2.58 (dd, J=17.8, 11.1 Hz, 2H), 2.03 (d, J=22.6 Hz, 5H), 1.80-1.70 (m, 2H), 1.56 (dd, J=22.7, 11.5 Hz, 2H).

### Example 89

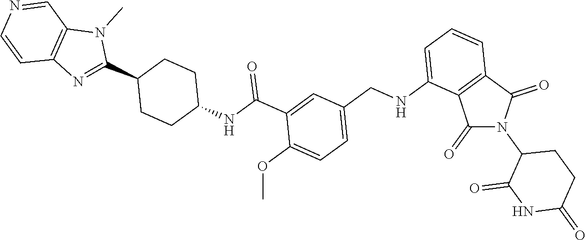 [SMILES: COc1ccc(CNc2cccc3c2C(=O)N(C2CCC(=O)NC2=O)C3=O)cc1C(=O)N[C@H]1CC[C@H](c2nc3ccncc3n2C)CC1]

### Preparation Steps

### Step 1

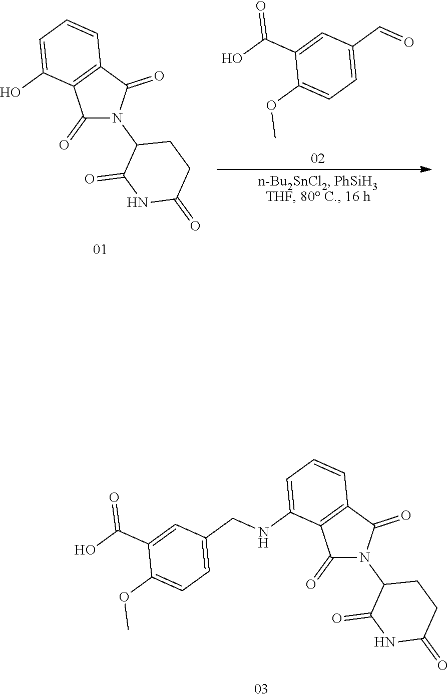 [SMILES: O=C1CCC(N2C(=O)c3cccc(O)c3C2=O)C(=O)N1 ; COc1ccc(C=O)cc1C(=O)O ; "02" ; "n-Bu2SnCl2, PhSiH3THF, 80° C., 16 h" ; COc1ccc(CNc2cccc3c2C(=O)N(C2CCC(=O)NC2=O)C3=O)cc1C(=O)O]

In a 10 mL microwave tube, 01 (253 mg, 0.93 mmol) and 02 (200 mg, 1.10 mmol) were dissolved in tetrahydrofuran (5 mL), and PhSiH₃ (100 mg, 0.93 mmol) and n-Bu₂SnCl₂ (336 mg, 1.11 mmol) were added. The mixture was reacted in a sealed tube at 80° C. for 16 h, and cooled to room temperature, and water (20 mL) was added. The reaction mixture was extracted with ethyl acetate (20 mL×3), and the organic phase was concentrated to dryness by rotary evaporation to give a crude product, which was then purified by pre-TLC to give the target product (360 mg, 89.1% yield). LC-MS: 438.0 [M+H]⁺.

### Step 2

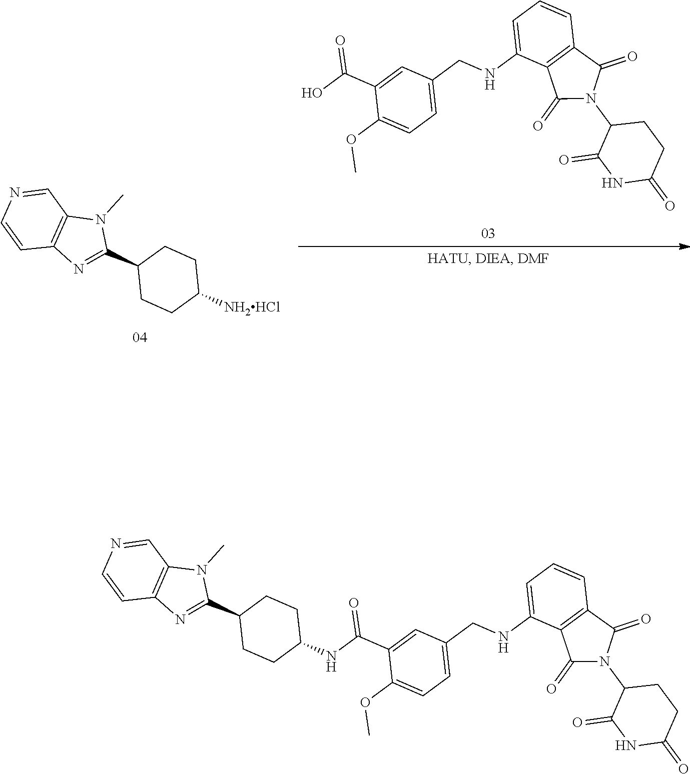

04 (100 mg, 0.37 mmol), 03 (165 mg, 0.37 mmol) and N,N-diisopropylethylamine (146 mg, 1.13 mmol) were dissolved in N,N-dimethylformamide (2 mL) at room temperature, and (7-azabenzotriazole)-tetramethyluronium hexafluorophosphate (172 mg, 0.45 mmol) was added with stirring at room temperature. The reaction mixture was stirred at room temperature for 2 h. The mixture was purified by preparative chromatography (C18, 5-100% acetonitrile/water as mobile phase, 0.03% hydrochloric acid) followed by lyophilization to give the target product as a yellow solid (in the form of a hydrochloride salt, 34.78 mg, 13.7% yield). LC-MS: [M+H]⁺ 650.4.

¹H NMR (400 MHz, DMSO-d₆) δ 11.11 (s, 1H), 9.47 (s, 1H), 8.57 (s, 1H), 8.18 (s, 1H), 8.06 (s, 1H), 7.71 (s, 1H), 7.50 (d, J=10.8 Hz, 2H), 7.24 (s, 1H), 7.12 (d, J=7.2 Hz, 1H), 7.01 (d, J=8.7 Hz, 2H), 5.06 (s, 1H), 4.53 (s, 2H), 4.03 (s, 3H), 3.87 (s, 3H), 3.23-3.20 (m, 1H), 2.90 (s, 1H), 2.58 (s, 2H), 2.06 (s, 6H), 1.78 (d, J=12.1 Hz, 2H), 1.56 (s, 2H).

### Example 90

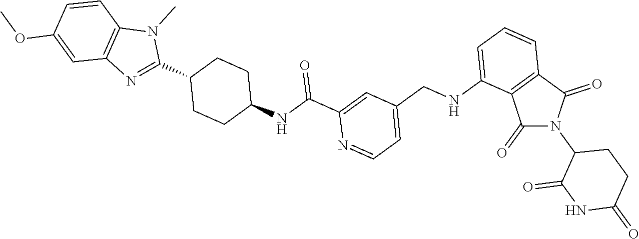 [SMILES: COc1ccc2c(c1)nc([C@H]1CC[C@H](NC(=O)c3cc(CNc4cccc5c4C(=O)N(C4CCC(=O)NC4=O)C5=O)ccn3)CC1)n2C]

### Preparation Steps

### Step 1

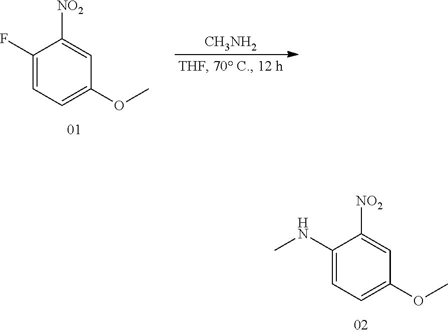

To a 100 mL single-neck flask were added 01 (1 g, 5.84 mmol), a solution of methylamine in ethanol (3.36 g, 29.21 mmol) and tetrahydrofuran (20 mL), and the mixture was reacted at 70° C. for 12 h, quenched with water (20 mL), and extracted with ethyl acetate (20 mL×2). The organic phase was washed with saturated brine (20 mL×3) and concentrated to dryness by rotary evaporation, and the residue was purified by column chromatography (ethyl acetate:petroleum ether=1:1) to give the target product as a red solid (800 mg, 75% yield). LC-MS: 183.1 [M+H]⁺.

### Step 2

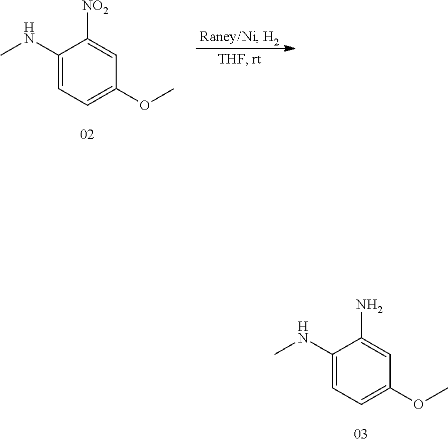

To a 100 mL single-neck flask were added 02 (600 mg, 3.29 mmol), Raney nickel (300 mg) and tetrahydrofuran (40 mL), and the mixture was reacted in a hydrogen atmosphere at room temperature for 2 h and filtered. The filtrate was concentrated to dryness by rotary evaporation to give the target product as a red solid (300 mg, 60% yield). LC-MS: 153.4 [M+H]⁺.

### Step 3

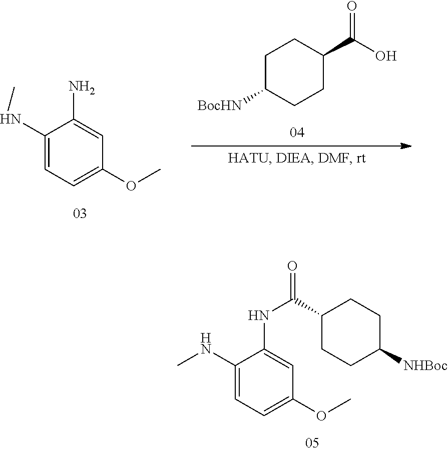

To a 100 mL single-neck flask were added 03 (250 mg, 1.64 mmol), 04 (399 mg, 1.64 mmol), HATU (810 mg, 2.13 mmol), DIEA (636 mg, 4.92 mmol) and DMF (10 mL), and the mixture was reacted at room temperature for 2 h, quenched with water (20 mL), and extracted with ethyl acetate (20 mL×2). The organic phase was washed with saturated brine (20 mL×3) and concentrated to dryness by rotary evaporation, and the residue was purified by column chromatography (dichloromethane:methanol=10:1) to give the target product as a white solid (400 mg, 65% yield). LC-MS: 378.2 [M+H]⁺.

### Step 4

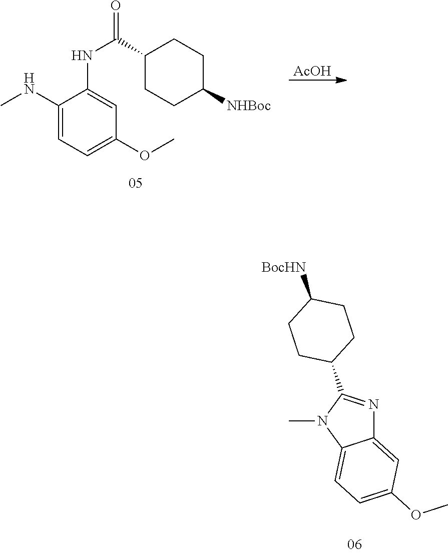 [SMILES: "AcOH" ; CNc1ccc(OC)cc1NC(=O)[C@H]1CC[C@H](NC(=O)OC(C)(C)C)CC1 ; COc1ccc2c(c1)nc([C@H]1CC[C@H](NC(=O)OC(C)(C)C)CC1)n2C]

In a 100 mL single-neck round-bottom flask, 05 (200 mg, 0.53 mmol) was dissolved in acetic acid (2 mL), and the mixture was reacted at 70° C. for 2.5 h, concentrated to dryness by rotary evaporation, diluted with ethyl acetate (20 mL×3), adjusted to pH 8-9 with saturated aqueous sodium bicarbonate solution (20 mL×2), washed with saturated brine (20 mL×3), dried, concentrated, and purified by column chromatography (dichloromethane:methanol=10:1) to give the target product 06 as a pale yellow solid (150 mg, 79% yield). LC-MS: 360.2 [M+H]⁺.

### Step 5

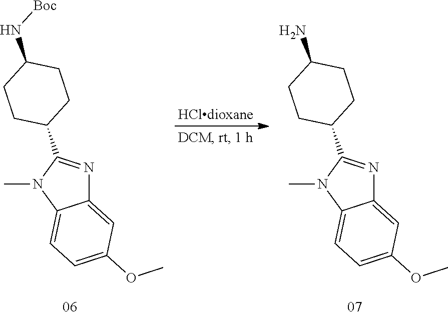 [SMILES: COc1ccc2c(c1)nc([C@H]1CC[C@H](NC(=O)OC(C)(C)C)CC1)n2C ; "DCM, rt, 1 h" ; "HCl·dioxane" ; COc1ccc2c(c1)nc([C@H]1CC[C@H](N)CC1)n2C]

In a 50 mL single-neck flask, 06 (50 mg, 0.139 mmol) was dissolved in dichloromethane (5 mL), and hydrochloric acid in 1,4-dioxane (4 mol/L, 0.5 mL) was added. The reaction mixture was stirred at room temperature for 1 h and could be directly used in the next step after being concentrated to dryness by rotary evaporation. LC-MS: 260.1 [M+H]⁺.

### Step 6

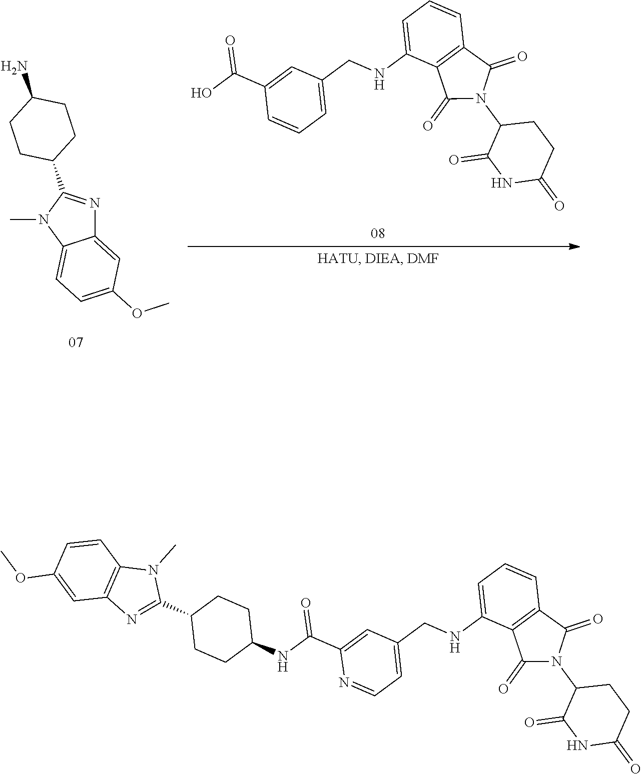 [SMILES: COc1ccc2c(c1)nc([C@H]1CC[C@H](N)CC1)n2C ; O=C1CCC(N2C(=O)c3cccc(NCc4cccc(C(=O)O)c4)c3C2=O)C(=O)N1 ; COc1ccc2c(c1)nc([C@H]1CC[C@H](NC(=O)c3cc(CNc4cccc5c4C(=O)N(C4CCC(=O)NC4=O)C5=O)ccn3)CC1)n2C]

07 (35 mg, 0.135 mmol) was dissolved in N,N-dimethylformamide (1 mL) at room temperature, and 08 (55 mg, 0.135 mmol), HATU (76 mg, 0.2 mmol) and N,N-diisopropylethylamine (87 mg, 0.673 mmol) were added successively. The above mixture was stirred at room temperature for 1 h. The reaction mixture was purified by preparative high performance liquid chromatography to give a yellowish-green solid (39.08 mg, 45% yield). LC-MS: 650.3 [M+H]⁺.

¹H NMR (400 MHz, DMSO-d6) δ 11.11 (s, 1H), 8.69 (d, J=8.4 Hz, 1H), 8.60 (d, J=5.0 Hz, 1H), 8.04 (s, 1H), 7.88 (d, J=9.0 Hz, 1H), 7.59 (d, J=4.9 Hz, 1H), 7.53-7.45 (m, 2H), 7.24-7.19 (m, 2H), 7.06 (d, J=7.1 Hz, 1H), 6.87 (d, J=8.6 Hz, 1H), 5.10 (dd, J=12.8, 5.4 Hz, 1H), 4.72 (d, J=5.8 Hz, 2H), 4.01 (s, 3H), 3.88 (s, 3H), 3.29 (t, J=12.0 Hz, 1H), 2.97-2.85 (m, 1H), 2.64-2.53 (m, 2H), 2.18-1.95 (m, 6H), 1.81 (dd, J=24.6, 12.0 Hz, 2H), 1.69 (dd, J=23.2, 11.2 Hz, 2H).

### Example 91

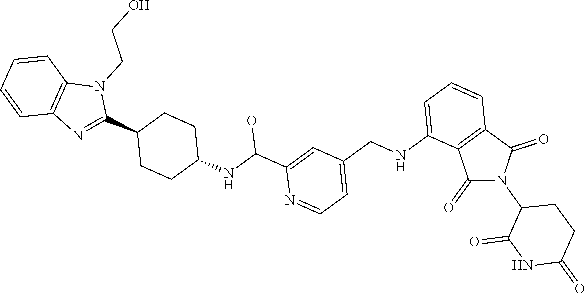

### Preparation Steps

### Step 1

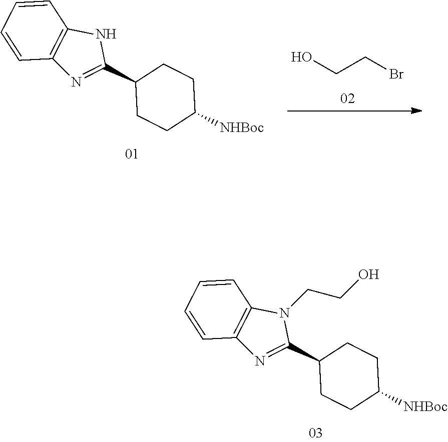

To a sealed tube were added at room temperature 01 (100 mg, 0.317 mmol), 02 (51.5 mg, 0.412 mmol), cesium carbonate (145 mg, 0.444 mmol) and N,N-dimethylformamide (3 mL). The above mixture was stirred under closed conditions at 100° C. for 16 h and filtered. The filtrate was passed through a reversed-phase column (C18, 5-100% acetonitrile/water as mobile phase, 0.03% trifluoroacetic acid) to separate the target product as a colorless oil (130 mg, crude). LC-MS: 360.4 [M+H]⁺.

### Step 2

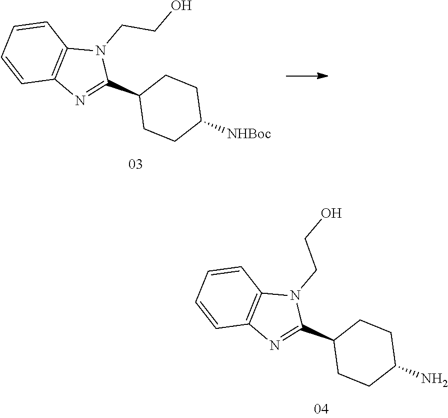 [SMILES: CC(C)(C)OC(=O)N[C@H]1CC[C@H](c2nc3ccccc3n2CCO)CC1 ; N[C@H]1CC[C@H](c2nc3ccccc3n2CCO)CC1]

03 (130 mg, 0.362 mmol) was dissolved in methanol (2 mL) at room temperature, and a solution of hydrogen chloride in dioxane (2 mL) was added. The above mixture was stirred at room temperature for 2 h and concentrated to dryness by rotary evaporation to give the target product as a brownish-yellow oil (34 mg, 41.3% yield over two steps). LC-MS: 260.4 [M+H]⁺.

### Step 3

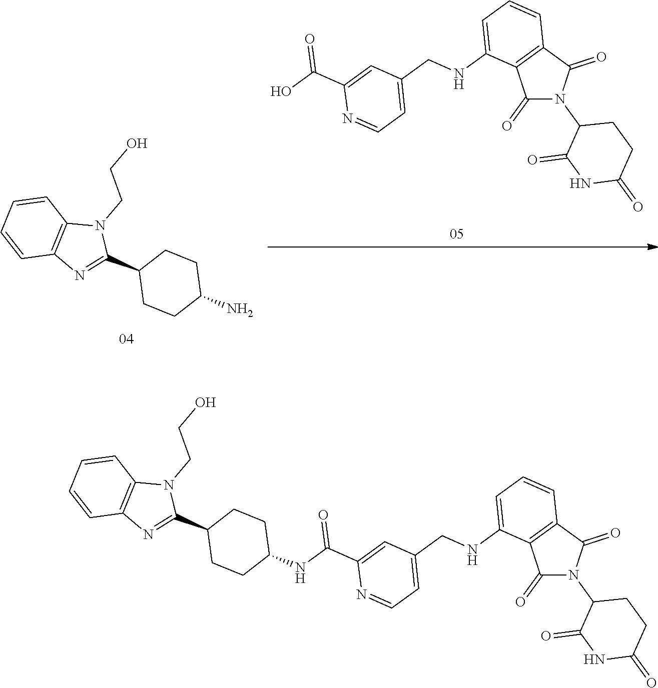 [SMILES: N[C@H]1CC[C@H](c2nc3ccccc3n2CCO)CC1 ; O=C1CCC(N2C(=O)c3cccc(NCc4ccnc(C(=O)O)c4)c3C2=O)C(=O)N1 ; O=C1CCC(N2C(=O)c3cccc(NCc4ccnc(C(=O)N[C@H]5CC[C@H](c6nc7ccccc7n6CCO)CC5)c4)c3C2=O)C(=O)N1]

04 (34.0 mg, 0.131 mmol), 05 (53.5 mg, 0.131 mmol) and NN-diisopropylethylamine (50.8 mg, 0.393 mmol) were mixed in anhydrous N,N-dimethylformamide (1 mL) at room temperature, and (7-azabenzotriazole)-tetramethyluronium hexafluorophosphate (74.9 mg, 0.197 mmol) was added with stirring at room temperature. The reaction mixture was stirred at room temperature for 2 h. The mixture was purified by preparative chromatography (C18, 5-100% acetonitrile/water as mobile phase, 0.1% formic acid) to separate the target product as a yellow solid (22.42 mg, 24.5% yield). LC-MS: 650.3 [M+H]⁺. ¹H NMR (400 MHz, DMSO-d₆) δ 11.17 (s, 1H), 8.67-8.60 (m, 2H), 8.10 (s, 1H), 7.65-7.59 (m, 2H), 7.58-7.52 (m, 3H), 7.26-7.17 (m, 2H), 7.11 (d, J=7.0 Hz, 1H), 6.93 (d, J=8.6 Hz, 1H), 5.15 (dd, J=12.8, 5.3 Hz, 1H), 5.04 (t, J=5.3 Hz, 1H), 4.77 (d, J=6.4 Hz, 2H), 4.33 (t, J=5.0 Hz, 2H), 3.99-3.85 (m, 1H), 3.77 (dd, J=10.5, 5.2 Hz, 2H), 3.13-3.02 (m, 1H), 3.00-2.89 (m, 1H), 2.70-2.58 (m, 2H), 2.17-2.11 (m, 1H), 2.09-1.97 (m, 4H), 1.90-1.78 (m, 2H), 1.73-1.62 (m, 2H).

### Example 92

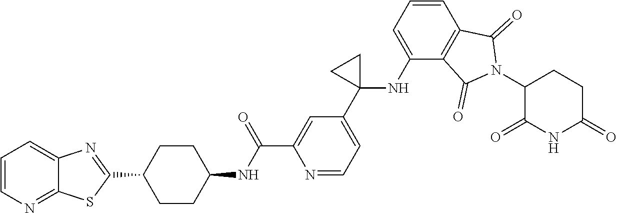 [SMILES: O=C1CCC(N2C(=O)c3cccc(NC4(c5ccnc(C(=O)N[C@H]6CC[C@H](c7nc8cccnc8s7)CC6)c5)CC4)c3C2=O)C(=O)N1]

### Preparation Steps

### Step 1

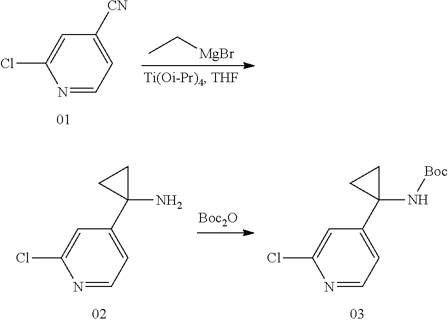

01 (5 g, 36.09 mmol) was dissolved in tetrahydrofuran (50 mL) at room temperature, and tetraisopropyl titanate (15.4 g, 54.14 mmol) was added at 0° C. The mixture was stirred for 10 min, and then ethylmagnesium bromide (36 mL, 108.26 mmol) was added. The mixture was stirred for 10 min, slowly heated to room temperature, stirred for 4 h, quenched with water, and then stirred for 0.5 h. Di-tert-butyl dicarbonate (7.87 g, 39.1 mmol) was added, and the mixture was reacted for 16 h. The reaction mixture was diluted with water (75 mL), and extracted with ethyl acetate (100 mL×3). The organic phases were washed with saturated brine (30 mL×3), combined, dried over anhydrous sodium sulfate, and concentrated to give a crude product, which was then purified by column chromatography (petroleum ether:ethyl acetate=2:1) to give a yellow oil (330 mg, 3.4% yield). LC-MS: 269.2 [M+H]⁺.

### Step 2

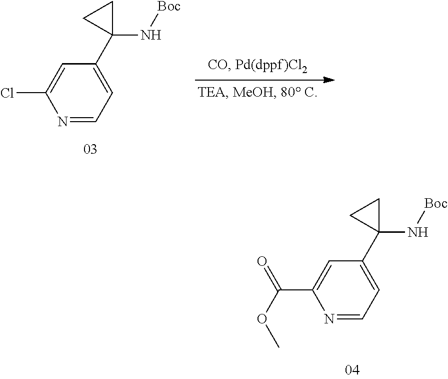 [SMILES: CC(C)(C)OC(=O)NC1(c2ccnc(Cl)c2)CC1 ; COC(=O)c1cc(C2(NC(=O)OC(C)(C)C)CC2)ccn1]

03 (400 mg, 1.49 mmol) was dissolved in methanol (10 mL) at room temperature, and triethylamine (453 mg, 4.48 mmol) and [1,1′-bis(diphenylphosphino)ferrocene]palladium dichloride (109 mg, 0.14 mmol) were added. The mixture was stirred in a carbon monoxide atmosphere at 80° C. for 16 h. After being cooled to room temperature, the reaction mixture was diluted with water (10 mL) and extracted with ethyl acetate (25 mL×3). The organic phases were washed with saturated brine (10 mL×3), combined, dried over anhydrous sodium sulfate, and concentrated to give a crude product, which was then purified by column chromatography (petroleum ether:ethyl acetate=1:1) to give a yellow oil (200 mg, 45.9% yield). LC-MS: 293.4 [M+H]⁺.

### Step 3

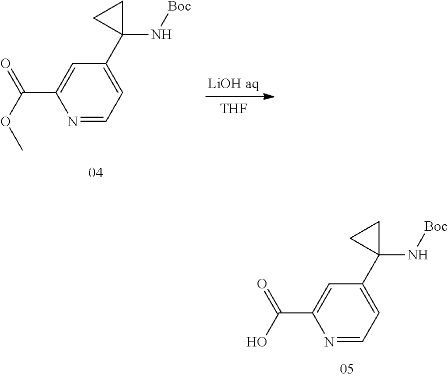 [SMILES: COC(=O)c1cc(C2(NC(=O)OC(C)(C)C)CC2)ccn1 ; CC(C)(C)OC(=O)NC1(c2ccnc(C(=O)O)c2)CC1 ; "THF"]

04 (200 mg, 0.68 mmol) was dissolved in tetrahydrofuran (2 mL) at room temperature, and saturated aqueous lithium hydroxide solution (1 mL) was added. The mixture was stirred in a nitrogen atmosphere at 35° C. for 16 h and concentrated to give a yellow solid (189 mg, crude).

LC-MS: 279.2 [M+H]⁺.

### Step 4

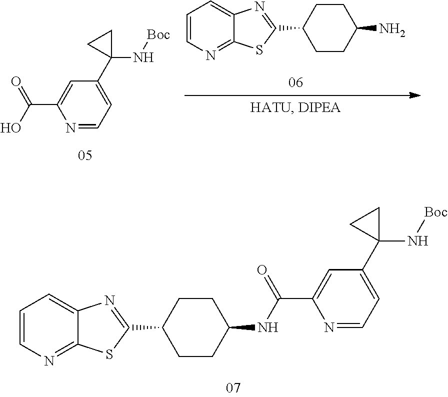

05 (189 mg, 0.68 mmol) was dissolved in N,N-dimethylformamide (2 mL) at room temperature, and 06 (158 mg, 0.68 mmol), 2-(7-azabenzotriazol)-N,N,N,N-tetramethyluronium hexafluorophosphate (517 mg, 1.36 mmol) and N,N-diisopropylethylamine (263 mg, 2.04 mmol) were added. The mixture was stirred in a nitrogen atmosphere at room temperature for 2 h. The reaction mixture was diluted with water (10 mL) and extracted with ethyl acetate (25 mL×3), and the organic phases were washed with saturated brine (10 mL×3), combined, dried over anhydrous sodium sulfate, and concentrated to give a crude product, which was then purified by column chromatography (petroleum ether:ethyl acetate=1:1) to give a yellow oil (240 mg, 71.4% yield). LC-MS: 494.2 [M+H]⁺.

### Step 5

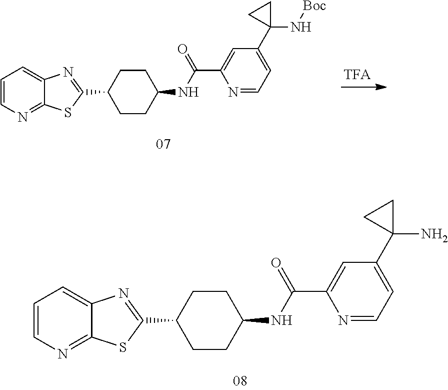 [SMILES: CC(C)(C)OC(=O)NC1(c2ccnc(C(=O)N[C@H]3CC[C@H](c4nc5cccnc5s4)CC3)c2)CC1 ; NC1(c2ccnc(C(=O)N[C@H]3CC[C@H](c4nc5cccnc5s4)CC3)c2)CC1]

To a 50 mL single-neck flask were added 07 (120 mg, 0.243 mmol), trifluoroacetic acid (2.5 mL) and dichloromethane (2.5 mL), and the mixture was reacted at room temperature for 12 h and concentrated to dryness by rotary evaporation. The residue was purified by reversed-phase column chromatography (acetonitrile/water=46%) to give a colorless transparent liquid (46 mg, 48.2% yield). LC-MS: 394.3 [M+H]⁺.

### Step 6

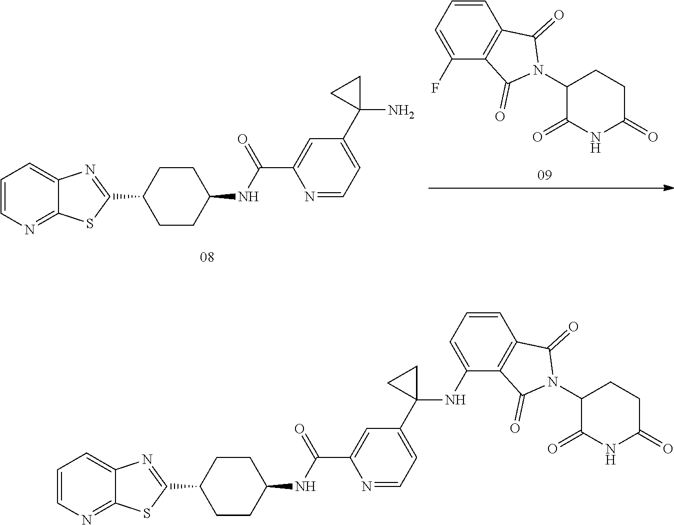 [SMILES: NC1(c2ccnc(C(=O)N[C@H]3CC[C@H](c4nc5cccnc5s4)CC3)c2)CC1 ; O=C1CCC(N2C(=O)c3cccc(F)c3C2=O)C(=O)N1 ; O=C1CCC(N2C(=O)c3cccc(NC4(c5ccnc(C(=O)N[C@H]6CC[C@H](c7nc8cccnc8s7)CC6)c5)CC4)c3C2=O)C(=O)N1]

To a 50 mL single-neck flask were added 08 (46 mg, 0.117 mmol), 09 (48 mg, 0.117 mmol), N,N-diisopropylethylamine (45.36 mg, 0.351 mmol) and dimethyl sulfoxide (1.5 mL), and the mixture was reacted at 140° C. for 16 h. The reaction mixture was purified by preparative high performance liquid chromatography to give a green solid (3.68 mg, 4.8% yield). LC-MS: 650.2 [M+H]⁺.

¹H NMR (400 MHz, DMSO-d₆) δ 8.60-8.54 (m, 2H), 8.52-8.46 (m, 1H), 8.34 (dd, J=8.2, 1.4 Hz, 1H), 7.80 (d, J=11.4 Hz, 1H), 7.59-7.48 (m, 3H), 7.38-7.30 (m, 1H), 7.13 (d, J=7.1 Hz, 1H), 6.87 (t, J=8.2 Hz, 1H), 5.11 (dd, J=12.8, 5.4 Hz, 1H), 3.85 (s, 1H), 3.12 (s, 1H), 2.89 (d, J=12.4 Hz, 1H), 2.64-2.55 (m, 2H), 2.34 (d, J=7.9 Hz, 2H), 2.22 (d, J=11.3 Hz, 2H), 2.09 (s, 1H), 2.03-1.92 (m, 3H), 1.75 (s, 1H), 1.68 (d, J=8.7 Hz, 2H), 1.61 (s, 1H), 1.51-1.47 (m, 1H).

### Example 93

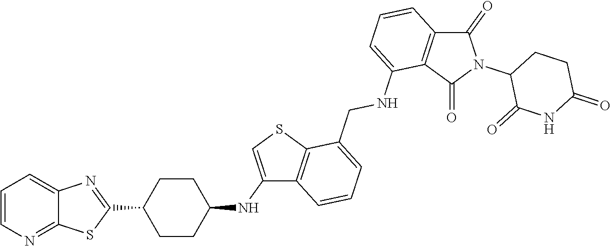 [SMILES: O=C1CCC(N2C(=O)c3cccc(NCc4cccc5c(N[C@H]6CC[C@H](c7nc8cccnc8s7)CC6)csc45)c3C2=O)C(=O)N1]

### Preparation Steps

### Step 1

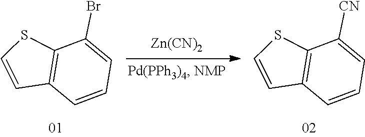

01 (500 mg, 2.35 mmol), zinc cyanide (276 mg, 2.35 mmol) and tetrakis(triphenylphosphine)palladium (272 mg, 0.235 mmol) were mixed in N-methylpyrrolidone (10 mL) at room temperature, and the mixture was purged with argon three times, stirred in an argon atmosphere at 130° C. for 3 h, and cooled to room temperature. Water (100 mL) was added, followed by extraction with ethyl acetate (30 mL×3). The organic phase was washed with saturated brine (30 mL×2), dried, and filtered, and the filtrate was concentrated to dryness by rotary evaporation. The residue was passed through a flash silica gel column (petroleum ether:ethyl acetate=20:1) to separate the target product as a white solid (344 mg, 83.7% yield). ¹H NMR (400 MHz, DMSO) δ 8.30 (d, J=8.1 Hz, 1H), 8.05 (d, J=5.4 Hz, 1H), 7.99 (d, J=7.4 Hz, 1H), 7.69 (d, J=5.4 Hz, 1H), 7.63 (t, J=7.8 Hz, 1H).

### Step 2

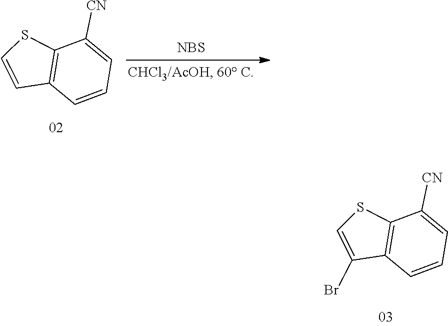

02 (344 mg, 2.16 mmol) was dissolved in chloroform (5 mL) and acetic acid (5 mL) at room temperature, and N-bromosuccinimide (500 mg, 2.81 mmol) was added at room temperature. The reaction mixture was stirred at 60° C. for 16 h, cooled to room temperature, and concentrated. Water (60 mL) was added, followed by extraction with ethyl acetate (20 mL×3). The organic phase was washed with saturated brine (60 mL), dried over anhydrous sodium sulfate, and filtered, and the filtrate was concentrated to dryness by rotary evaporation. The residue was passed through a flash silica gel column (petroleum ether:ethyl acetate=5:1) to separate the target product as a white solid (234 mg, 45.5% yield). ¹H NMR (400 MHz, DMSO) δ 8.23 (s, 1H), 8.10-8.04 (m, 2H), 7.73-7.67 (m, 1H).

### Step 3

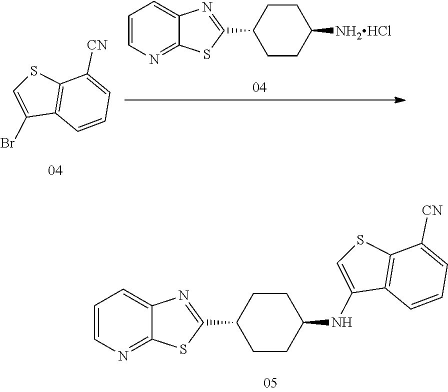

To a sealed tube were added at room temperature 03 (140 mg, 0.588 mmol), 04 (159 mg, 0.588 mmol), cuprous iodide (16.8 mg, 0.0882 mmol), 2,2,6,6-tetramethyl-3,5-heptanedione (32.5 mg, 0.176 mmol), cesium carbonate (575 mg, 1.76 mmol) and N-methylpyrrolidone (5 mL). The mixture was purged with argon for one minute, and then the tube was sealed. The reaction mixture was stirred at 100° C. for 14 h and cooled to room temperature. Then water (50 mL) was added, followed by extraction with ethyl acetate (10 mL×3). The organic phase was washed with saturated brine (20 mL), dried over anhydrous sodium sulfate, and filtered, and the filtrate was concentrated to dryness by rotary evaporation. The residue was passed through a flash silica gel column (petroleum ether:ethyl acetate=1:1) to separate the target product as a yellow solid (15 mg, 6.5% yield). LC-MS: 391.3 [M+H]⁺.

### Step 4

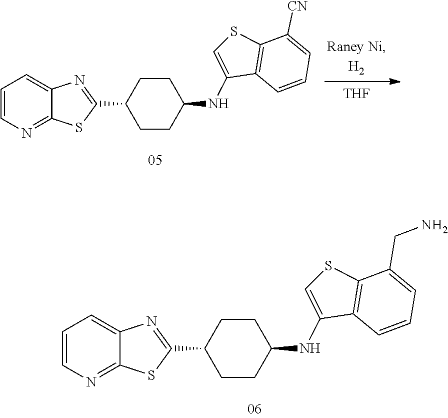 [SMILES: N#Cc1cccc2c(N[C@H]3CC[C@H](c4nc5cccnc5s4)CC3)csc12 ; NCc1cccc2c(N[C@H]3CC[C@H](c4nc5cccnc5s4)CC3)csc12 ; "THF"]

05 (15 mg, 0.0384 mmol) was dissolved in tetrahydrofuran (0.5 mL) and methanol (0.5 mL), and Raney nickel (15 mg) and ammonia solution (0.2 mL) were added. After being purged with hydrogen three times, the reaction mixture was stirred in a hydrogen atmosphere at room temperature for 14 h and filtered. The filtrate was concentrated to dryness by rotary evaporation to give the target product as a brown oil (15 mg, crude). LC-MS: 395.3 [M+H]⁺.

### Step 5

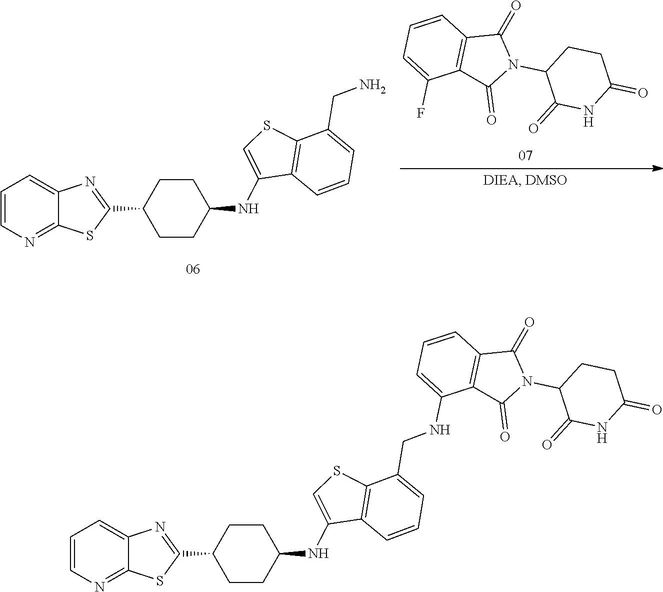 [SMILES: NCc1cccc2c(N[C@H]3CC[C@H](c4nc5cccnc5s4)CC3)csc12 ; O=C1CCC(N2C(=O)c3cccc(F)c3C2=O)C(=O)N1 ; O=C1CCC(N2C(=O)c3cccc(NCc4cccc5c(N[C@H]6CC[C@H](c7nc8cccnc8s7)CC6)csc45)c3C2=O)C(=O)N1]

06 (15 mg, 0.0380 mmol), 07 (8.40 mg, 0.0304 mmol) and N,N-diisopropylethylamine (9.82 mg, 0.0760 mmol) were dissolved in anhydrous dimethyl sulfoxide (0.5 mL), and the reaction mixture was stirred at 120° C. for 12 h. The mixture was purified by preparative chromatography (C18, 5-100% acetonitrile/water as mobile phase, 0.1% formic acid) to separate the target product as a yellow solid (2 mg, 8.1% yield). LC-MS: 651.2 [M+H]⁺, ¹H NMR (400 MHz, DMSO) δ 11.12 (s, 1H), 8.19-8.13 (m, 1H), 8.13-8.04 (m, 1H), 7.63-7.48 (m, 3H), 7.45-7.35 (m, 3H), 7.24-7.15 (m, 1H), 7.07 (d, J=7.0 Hz, 1H), 6.99 (d, J=8.7 Hz, 1H), 5.10 (dd, J=12.9, 5.3 Hz, 1H), 4.89 (d, J=5.9 Hz, 2H), 3.75-3.67 (m, 1H), 3.01-2.86 (m, 2H), 2.65-2.54 (m, 2H), 2.10-2.04 (m, 1H), 1.95-1.61 (m, 9H).

### Example 94

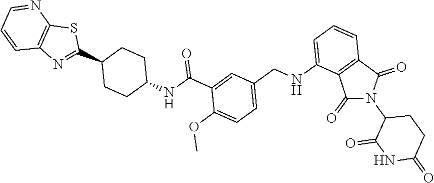 [SMILES: COc1ccc(CNc2cccc3c2C(=O)N(C2CCC(=O)NC2=O)C3=O)cc1C(=O)N[C@H]1CC[C@H](c2nc3cccnc3s2)CC1]

### Preparation Steps

### Step 1

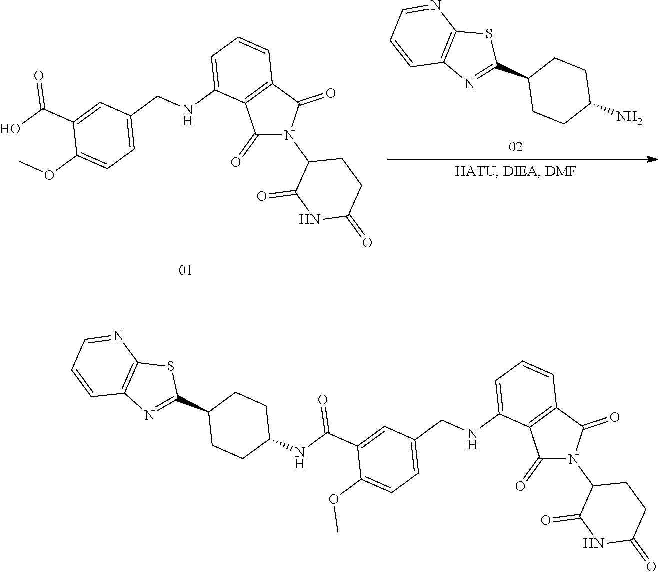 [SMILES: COc1ccc(CNc2cccc3c2C(=O)N(C2CCC(=O)NC2=O)C3=O)cc1C(=O)O ; N[C@H]1CC[C@H](c2nc3cccnc3s2)CC1 ; COc1ccc(CNc2cccc3c2C(=O)N(C2CCC(=O)NC2=O)C3=O)cc1C(=O)N[C@H]1CC[C@H](c2nc3cccnc3s2)CC1]

To a 50 mL single-neck flask were added 01 (75 mg, 0.17 mmol), 02 (40 mg, 0.17 mmol), HATU (98 mg, 0.26 mmol), DIPEA (66 mg, 0.52 mmol) and DMF (2 mL), and the mixture was stirred at room temperature for 16 h and purified by preparative reversed-phase chromatography to give the product as a yellow solid (24.71 mg, 22.3% yield). LCMS 653.1 [M+H]^{+ 1}HNMR (400 MHz, DMSO-d₆) δ 11.09 (s, 1H), 8.58 (d, J=4.7 Hz, 1H), 8.34 (d, J=8.2 Hz, 1H), 8.02 (d, J=7.7 Hz, 1H), 7.73 (d, J=1.9 Hz, 1H), 7.58-7.53 (m, 1H), 7.49 (dd, J=16.5, 8.1 Hz, 2H), 7.23 (t, J=6.2 Hz, 1H), 7.11 (d, J=8.6 Hz, 1H), 7.01 (t, J=6.0 Hz, 1H), 6.98 (d, J=8.7 Hz, 1H), 5.07 (dd, J=12.9, 5.3 Hz, 1H), 4.52 (d, J=6.0 Hz, 2H), 3.87 (s, 3H), 3.83 (s, 1H), 3.15 (t, J=11.6 Hz, 2H), 2.88 (dd, J=16.4, 9.5 Hz, 1H), 2.57 (dd, J=18.6, 10.5 Hz, 2H), 2.23 (d, J=11.8 Hz, 2H), 2.12-1.99 (m, 3H), 1.74 (dd, J=16.5, 10.6 Hz, 2H), 1.51 (dd, J=14.8, 11.0 Hz, 2H).

### Example 95

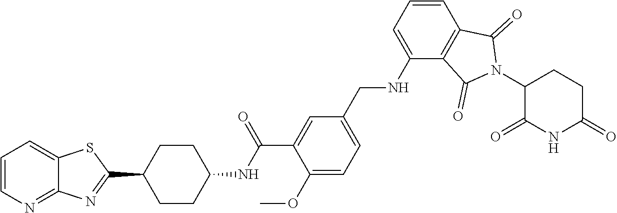 [SMILES: COc1ccc(CNc2cccc3c2C(=O)N(C2CCC(=O)NC2=O)C3=O)cc1C(=O)N[C@H]1CC[C@H](c2nc3ncccc3s2)CC1]

### Preparation Steps

### Step 1

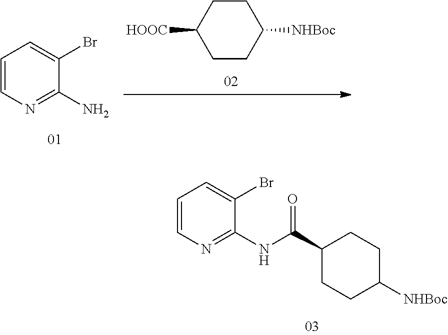

01 (2.00 g, 11.6 mmol), 02 (1.88 g, 7.73 mmol) and 4-dimethylaminopyridine (1.22 g, 10.0 mmol) were dissolved in anhydrous N,N-dimethylformamide (30 mL), and 1-(3-dimethylaminopropyl)-3-ethylcarbodiimide hydrochloride (1.92 g, 10.0 mmol) was added with stirring at room temperature. The reaction mixture was stirred at room temperature for 7 days. Water (20 mL) was added, followed by filtration to give the target product as a gray solid (1.65 g, 35.8% yield). LC-MS: 398.2 [M+H]⁺.

### Step 2

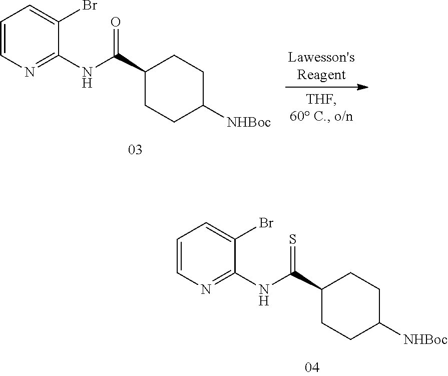

03 (600 mg, 1.51 mmol) and Lawesson's reagent (918 mg, 2.27 mmol) were dissolved in anhydrous tetrahydrofuran (15 mL), and the reaction mixture was stirred at 60° C. for 16 h. Water (100 mL) was added, followed by extraction with ethyl acetate (30 mL×3). The organic phase was washed with saturated brine (50 mL), dried over anhydrous sodium sulfate, and filtered, and the filtrate was concentrated to dryness by rotary evaporation. The residue was purified through a flash silica gel column (petroleum ether:ethyl acetate=2:1) and a reversed-phase column (C18, 10-100% acetonitrile/water as mobile phase, 0.1% formic acid) to give the target product as a white solid (386 mg, 61.8% yield). LC-MS: 414.2 [M+H]⁺.

### Step 3

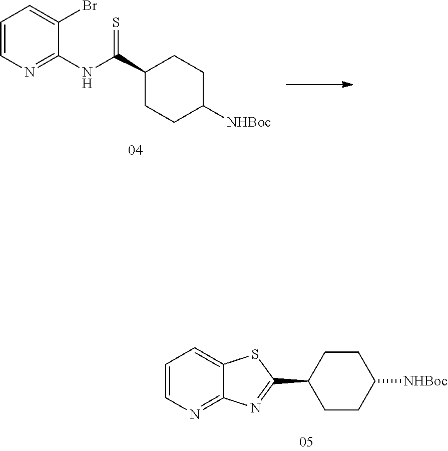

To a sealed tube were added 04 (310 mg, 0.748 mmol), cuprous iodide (14.2 mg, 0.075 mmol), 1,10-phenanthroline (30.0, 0.150 mmol), cesium carbonate (487 mg, 1.50 mmol) and ethylene glycol dimethyl ether (15 mL), and the reaction mixture was stirred at 90° C. for 16 h and filtered. The residue was purified through a flash silica gel column (petroleum ether:ethyl acetate=0-100%) and a reversed-phase column (C18, 10-100% acetonitrile/water as mobile phase, 0.03% trifluoroacetic acid) to give the target product as a white solid (80.0 mg, 32.1% yield). LC-MS: 334.3 [M+H]⁺.

### Step 4

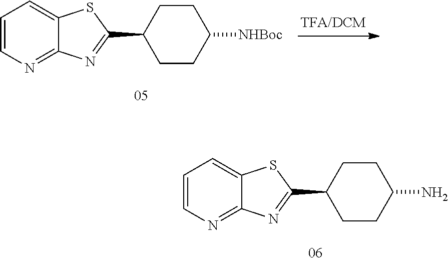 [SMILES: CC(C)(C)OC(=O)N[C@H]1CC[C@H](c2nc3ncccc3s2)CC1 ; N[C@H]1CC[C@H](c2nc3ncccc3s2)CC1]

05 (80.0 mg, 0.240 mmol) was dissolved in dichloromethane (2 mL), and trifluoroacetic acid (2 mL) was added with stirring at room temperature. The reaction mixture was stirred at room temperature for 1 h and concentrated under reduced pressure to remove the solvent to give the target product as a white solid (56.0 mg, crude). LC-MS: 234.2 [M+H]⁺.

### Step 5

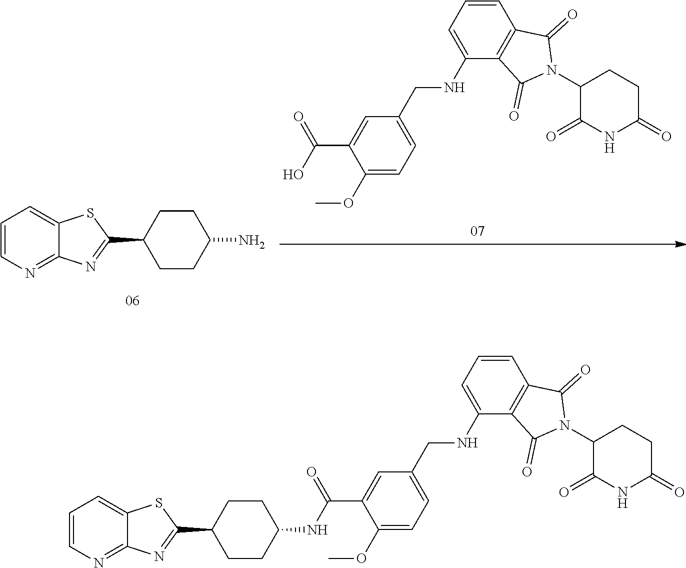 [SMILES: COc1ccc(CNc2cccc3c2C(=O)N(C2CCC(=O)NC2=O)C3=O)cc1C(=O)O ; N[C@H]1CC[C@H](c2nc3ncccc3s2)CC1 ; COc1ccc(CNc2cccc3c2C(=O)N(C2CCC(=O)NC2=O)C3=O)cc1C(=O)N[C@H]1CC[C@H](c2nc3ncccc3s2)CC1]

06 (56.0 mg, 0.240 mmol), 07 (105 mg, 0.240 mmol) and NN-diisopropylethylamine (124 mg, 0.960 mmol) were dissolved in anhydrous N,N-dimethylformamide (3 mL), and (7-azabenzotriazole)-tetramethyluronium hexafluorophosphate (137 mg, 0.360 mmol) was added in batches with stirring at room temperature. The reaction mixture was stirred at room temperature for 2 h. The mixture was purified by preparative reversed-phase liquid chromatography (C18, 5-100% acetonitrile/water as mobile phase, 0.1% formic acid) to separate the target product as a yellowish-green solid (52.62 mg, 33.6% yield). LC-MS: 653.3 [M+H]⁺, ¹H NMR (400 MHz, DMSO-d₆) δ 11.10 (s, 1H), 8.68-8.62 (m, 1H), 8.57 (dd, J=8.0, 1.4 Hz, 1H), 8.02 (d, J=7.7 Hz, 1H), 7.73 (d, J=1.7 Hz, 1H), 7.54-7.42 (m, 3H), 7.24 (t, J=6.3 Hz, 1H), 7.11 (d, J=8.6 Hz, 1H), 7.02 (d, J=7.0 Hz, 1H), 6.98 (d, J=8.6 Hz, 1H), 5.07 (dd, J=12.9, 5.2 Hz, 1H), 4.52 (d, J=5.4 Hz, 2H), 3.90-3.81 (m, 4H), 3.22-3.13 (m, 1H), 2.95-2.83 (m, 1H), 2.63-2.54 (m, 2H), 2.27-2.19 (m, 2H), 2.09-2.00 (m, 3H), 1.80-1.68 (m, 2H), 1.57-1.46 (m, 2H).

### Example 96

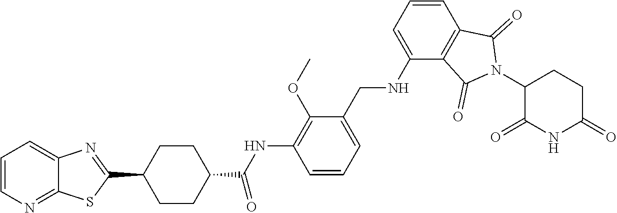 [SMILES: COc1c(CNc2cccc3c2C(=O)N(C2CCC(=O)NC2=O)C3=O)cccc1NC(=O)[C@H]1CC[C@H](c2nc3cccnc3s2)CC1]

### Preparation Steps

### Step 1

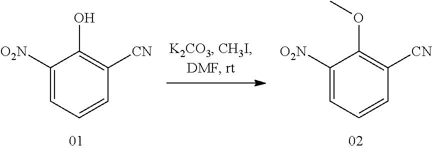

01 (500 mg, 3.05 mmol), potassium carbonate (1.69 g, 12.2 mmol) and iodomethane (1.73 g, 12.2 mmol) were dissolved in N,N-dimethylformamide (10 mL) at room temperature, and the reaction mixture was stirred at room temperature for 16 h. Water (50 mL) was added, followed by extraction with ethyl acetate (50 mL×3). The organic phase was washed with saturated sodium carbonate solution (50 mL×3) and saturated brine, dried over anhydrous sodium sulfate, and filtered, and the filtrate was concentrated to dryness by rotary evaporation to give the target product as a yellow solid (490 mg, 90.3% yield). ¹H NMR (400 MHz, CDCl₃) δ 8.03 (dd, J=8.2, 1.7 Hz, 1H), 7.83 (dd, J=7.8, 1.7 Hz, 1H), 7.31 (dd, J=16.2, 8.2 Hz, 1H), 4.20 (s, 3H).

### Step 2

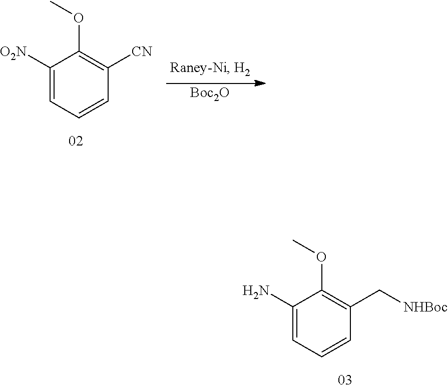 [SMILES: "Boc2O" ; COc1c(C#N)cccc1[N+](=O)[O-] ; COc1c(N)cccc1CNC(=O)OC(C)(C)C]

02 (150 mg, 0.842 mmol) was dissolved in methanol (10 mL) at room temperature, and Raney nickel (100 mg) and di-tert-butyl dicarbonate (202 mg, 0.926 mmol) were added at room temperature. The reaction mixture was purged with hydrogen twice, stirred in a hydrogen atmosphere at room temperature for 16 h, and filtered. The filtrate was concentrated to dryness by rotary evaporation, and the residue was passed through a silica gel column (petroleum ether:ethyl acetate=3:1) to separate the target product as a yellow oil (140 mg, 65.9% yield).

LC-MS: 197.0 [M+H−56]⁺.

### Step 3

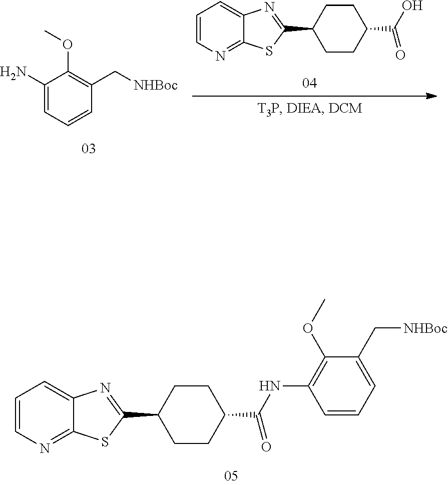 [SMILES: COc1c(N)cccc1CNC(=O)OC(C)(C)C ; O=C(O)[C@H]1CC[C@H](c2nc3cccnc3s2)CC1 ; COc1c(CNC(=O)OC(C)(C)C)cccc1NC(=O)[C@H]1CC[C@H](c2nc3cccnc3s2)CC1]

03 (140 mg, 0.555 mmol), 04 (146 mg, 0.555 mmol) and N,N-diisopropylethylamine (215 mg, 1.67 mmol) were dissolved in dichloromethane (10 mL) at room temperature, and 1-propylphosphonic anhydride (706 mg, 1.11 mmol, 50% in ethyl acetate) was added with stirring at room temperature. The reaction mixture was stirred at room temperature for 16 h. Water (100 mL) was added, followed by extraction with ethyl acetate (50 mL×3). The organic phase was washed with saturated brine (100 mL), dried over anhydrous sodium sulfate, and filtered, and the filtrate was concentrated to dryness by rotary evaporation. The residue was purified through a silica gel column (petroleum ether:ethyl acetate=2:1) to give the target product as a pink solid (70 mg, 25.4% yield). LC-MS: 497.1 [M+H]⁺.

### Step 4

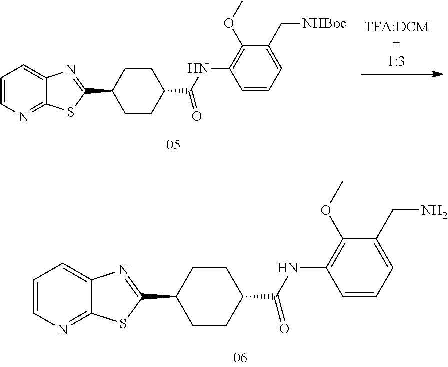 [SMILES: COc1c(CNC(=O)OC(C)(C)C)cccc1NC(=O)[C@H]1CC[C@H](c2nc3cccnc3s2)CC1 ; COc1c(CN)cccc1NC(=O)[C@H]1CC[C@H](c2nc3cccnc3s2)CC1]

05 (30 mg, 0.0604 mmol) was dissolved in dichloromethane (3 mL) at room temperature, and trifluoroacetic acid (1 mL) was added dropwise with stirring at room temperature. The reaction mixture was stirred at room temperature for 6 h and concentrated by rotary evaporation to remove the solvent to give the target product as a yellow oil (30 mg, crude). LC-MS: 397.1 [M+H]⁺.

### Step 5

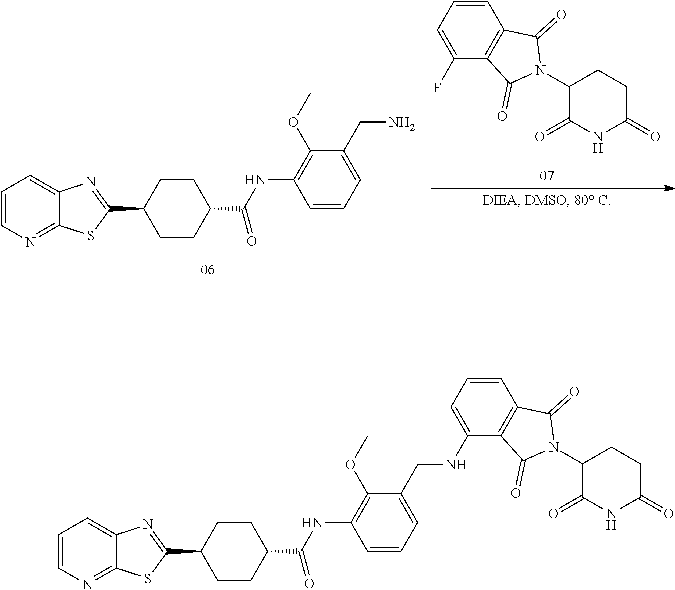 [SMILES: COc1c(CN)cccc1NC(=O)[C@H]1CC[C@H](c2nc3cccnc3s2)CC1 ; O=C1CCC(N2C(=O)c3cccc(F)c3C2=O)C(=O)N1 ; COc1c(CNc2cccc3c2C(=O)N(C2CCC(=O)NC2=O)C3=O)cccc1NC(=O)[C@H]1CC[C@H](c2nc3cccnc3s2)CC1]

06 (30 mg, 0.0757 mmol), 97 (20.9 mg, 0.0757 mmol) and NN-diisopropylethylamine (29.4 mg, 0.227 mmol) were dissolved in anhydrous dimethyl sulfoxide (2 mL) at room temperature, and the reaction mixture was stirred at 80° C. for 16 h. The mixture was cooled to 20° C., and purified by preparative chromatography (C18, 5-100% acetonitrile/water as mobile phase, 0.1% formic acid) to separate the target product as a yellow solid (4.97 mg, 10.1% yield). LC-MS: 653.2 [M+H]⁺. ¹H NMR (400 MHz, CDCl₃) δ 8.58 (s, 1H), 8.29 (d, J=8.5 Hz, 2H), 8.00 (d, J=7.7 Hz, 1H), 7.77 (s, 1H), 7.49 (dd, J=16.2, 8.8 Hz, 2H), 7.17-7.05 (m, 4H), 6.88 (d, J=8.5 Hz, 1H), 6.68 (s, 1H), 4.92 (dd, J=12.2, 5.4 Hz, 1H), 4.55 (s, 2H), 3.85 (s, 3H), 3.20 (s, 1H), 2.95-2.67 (m, 3H), 2.43 (d, J=10.9 Hz, 4H), 2.24 (d, J=9.9 Hz, 3H), 2.19-2.10 (m, 2H), 1.93-1.77 (m, 9H).

### Example 97

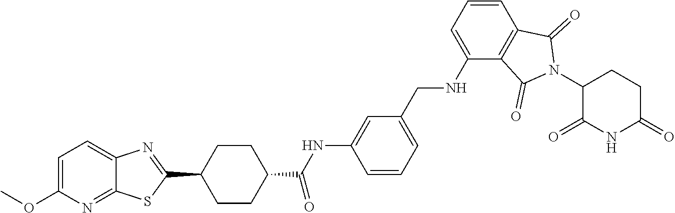 [SMILES: COc1ccc2nc([C@H]3CC[C@H](C(=O)Nc4cccc(CNc5cccc6c5C(=O)N(C5CCC(=O)NC5=O)C6=O)c4)CC3)sc2n1]

### Preparation Steps

### Step 1

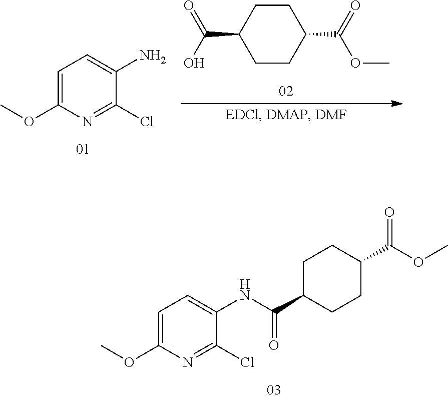

01 (1.257 g, 8.06 mmol), 02 (1.0 g, 5.37 mmol), 1-ethyl-3(3-dimethylpropylamine)carbodiimide (1.335 g, 6.98 mmol) and 4-dimethylaminopyridine (0.853 g, 6.98 mmol) were dissolved in N,N-dimethylformamide (10 mL). The reaction mixture was stirred at room temperature for 2 h. Water (60 mL) was added to the mixture, and a solid precipitated. The mixture was filtered, and the resulting filter cake was concentrated to dryness by rotary evaporation to give the target product as a white solid (1.587 g, 90.7% yield). LC-MS: 327.2 [M+H]⁺.

### Step 2

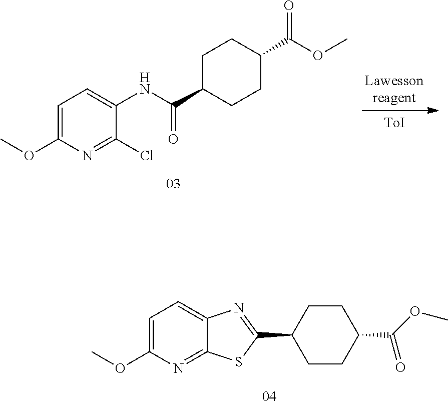

03 (800 mg, 2.45 mmol) and Lawesson's reagent (1485 mg, 3.68 mmol) were dissolved in toluene (8 mL) at room temperature, and the reaction mixture was stirred in an argon atmosphere at 120° C. for 16 h. Water (60 mL) was added, followed by extraction with ethyl acetate (50 mL×3). The organic phase was washed with saturated brine (100 mL), dried over anhydrous sodium sulfate, and filtered, and the filtrate was concentrated to dryness by rotary evaporation. The residue was purified through a silica gel column (petroleum ether:ethyl acetate=5:1) to give the target product as a white solid (461 mg, 61.5% yield). LC-MS: 307.1 [M+H]⁺.

### Step 3

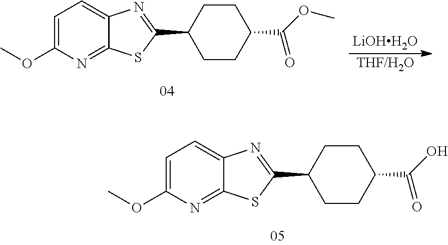

04 (100 mg, 0.33 mmol) was dissolved in tetrahydrofuran (2 mL) and water (2 mL) at room temperature, and lithium hydroxide monohydrate (35 mg, 0.83 mmol) was added at room temperature. The reaction mixture was stirred at room temperature for 3 h and concentrated to dryness by rotary evaporation. Water (20 mL) and ethyl acetate (20 mL) were added, and the aqueous phase was separated, adjusted to a pH value less than 7, and extracted with ethyl acetate (30 mL). The organic phase was concentrated to dryness by rotary evaporation to give the target product as a white solid (82 mg, 95.8% yield). LC-MS: 260.2 [M+H]⁺.

### Step 4

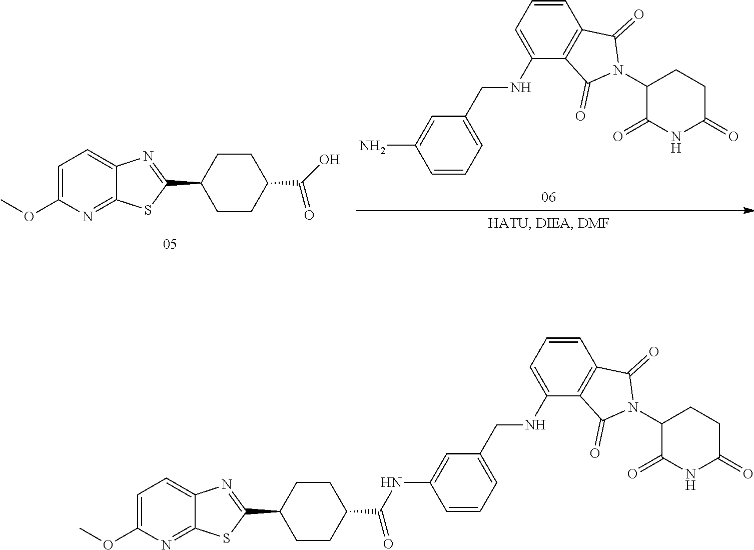 [SMILES: COc1ccc2nc([C@H]3CC[C@H](C(=O)O)CC3)sc2n1 ; Nc1cccc(CNc2cccc3c2C(=O)N(C2CCC(=O)NC2=O)C3=O)c1 ; COc1ccc2nc([C@H]3CC[C@H](C(=O)Nc4cccc(CNc5cccc6c5C(=O)N(C5CCC(=O)NC5=O)C6=O)c4)CC3)sc2n1]

05 (45 mg, 0.174 mmol), 06 (66 mg, 0.174 mmol) and N,N-diisopropylethylamine (67 mg, 0.522 mmol) were dissolved in N,N-dimethylformamide (1 mL) at room temperature, and (7-azabenzotriazole)-tetramethyluronium hexafluorophosphate (79 mg, 0.209 mmol) was added with stirring at room temperature. The reaction mixture was stirred at room temperature for 2 h. The mixture was purified by preparative chromatography (C18, 5-100% acetonitrile/water as mobile phase, 0.10% formic acid) to separate the target product as a yellow solid (26.72 mg, 23.6% yield). LC-MS: 653.3 [M+H]⁺. ¹H NMR (400 MHz, DMSO) δ 11.10 (s, 1H), 9.89 (s, 1H), 8.22 (d, J=8.9 Hz, 1H), 7.59 (d, J=8.4 Hz, 1H), 7.52 (dd, J=14.1, 6.2 Hz, 2H), 7.29-7.20 (m, 2H), 7.03 (d, J=7.1 Hz, 2H), 6.96 (d, J=8.8 Hz, 1H), 6.92 (d, J=8.6 Hz, 1H), 5.08 (dd, J=12.9, 5.3 Hz, 1H), 4.54 (d, J=6.0 Hz, 2H), 3.92 (s, 3H), 3.10 (s, 1H), 2.95-2.84 (m, 1H), 2.61 (dd, J=29.6, 13.4 Hz, 2H), 2.24 (s, 2H), 2.09-1.94 (m, 4H), 1.69-1.62 (m, 2H), 1.61-1.53 (m, 2H).

### Example 98

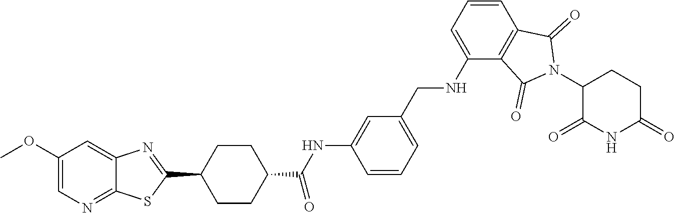 [SMILES: COc1cnc2sc([C@H]3CC[C@H](C(=O)Nc4cccc(CNc5cccc6c5C(=O)N(C5CCC(=O)NC5=O)C6=O)c4)CC3)nc2c1]

### Preparation Steps

### Step 1

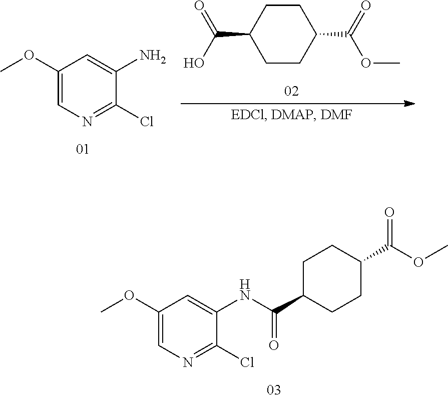

01 (250 mg, 1.582 mmol), 02 (196 mg, 1.055 mmol), 1-(3-dimethylaminopropyl)-3-ethylcarbodiimide hydrochloride (203 mg, 1.582 mmol) and 4-dimethylaminopyridine (128 mg, 1.582 mmol) were dissolved in N,N-dimethylformamide (2 mL) at room temperature, and the reaction mixture was stirred at 40° C. for 5 days. Water (100 mL) was added, followed by extraction with ethyl acetate (200 mL×3). The organic phase was washed with saturated brine (100 mL), dried over anhydrous sodium sulfate, and filtered, and the filtrate was concentrated to dryness by rotary evaporation. The residue was purified through a silica gel column (petroleum ether:ethyl acetate=4:1 to 3:1) to give the target product as a white solid (280 mg, 54.1%). LC-MS: 327.2 [M+H]⁺.

### Step 2

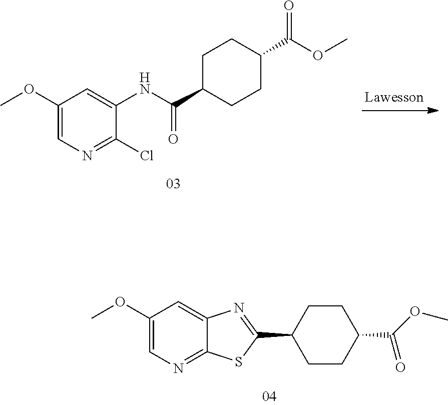

03 (250 mg, 0.767 mmol) was dissolved in tetrahydrofuran (10 mL) at room temperature, and Lawesson's reagent (404 mg, 1.0 mmol) was added. The reaction mixture was stirred at 60° C. for 16 h, and aqueous sodium bicarbonate solution (100 mL) was added, followed by extraction with ethyl acetate (200 mL×3). The organic phase was washed with saturated brine (100 mL), dried over anhydrous sodium sulfate, and filtered, and the filtrate was concentrated to dryness by rotary evaporation. The residue was purified through a silica gel column (petroleum ether:ethyl acetate=3:1 to 2:1) to give the target product as a white solid (180 mg, 76.9%). LC-MS: 307.1 [M+H]⁺.

### Step 3

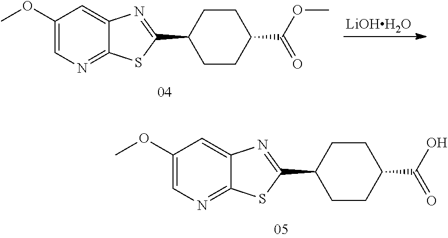

04 (150 mg, 0.490 mmol) was dissolved in tetrahydrofuran (3 mL) and methanol (3 mL) at room temperature, and lithium hydroxide monohydrate (40 mg, 0.980 mmol) dissolved in water (2 mL) was added. The reaction mixture was stirred at 50° C. for 2 h, concentrated by rotary evaporation to remove the solvent, adjusted to pH 11 with 0.5N HCl, and filtered to give the target product as a white solid (400 mg, crude). LC-MS: 294.1 [M+H]⁺.

### Step 4

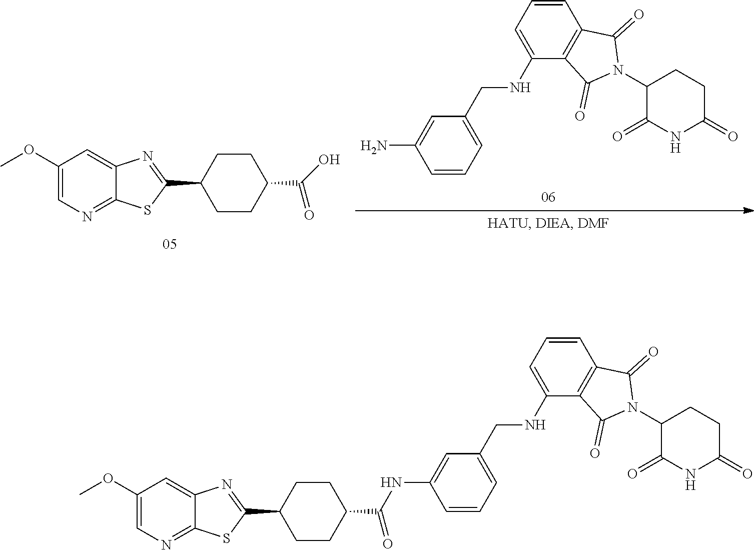 [SMILES: COc1cnc2sc([C@H]3CC[C@H](C(=O)O)CC3)nc2c1 ; Nc1cccc(CNc2cccc3c2C(=O)N(C2CCC(=O)NC2=O)C3=O)c1 ; COc1cnc2sc([C@H]3CC[C@H](C(=O)Nc4cccc(CNc5cccc6c5C(=O)N(C5CCC(=O)NC5=O)C6=O)c4)CC3)nc2c1]

05 (50 mg, 0.342 mmol), 06 (98 mg, 0.514 mmol), N,N-diisopropylethylamine (64 mg, 1.028 mmol) and 2-(7-azabenzotriazol)-N,N,N,N-tetramethyluronium hexafluorophosphate (195 mg, 1.028 mmol) were dissolved in N,N-dimethylformamide (2 mL) at room temperature, and the reaction mixture was stirred at room temperature for 16 h and purified by preparative chromatography (C18, 5-100% acetonitrile/water as mobile phase, 0.1% formic acid) to separate the target product as a yellow solid (19.30 mg, 17.1% yield). LC-MS: 653.5 [M+H]⁺. ¹H NMR (400 MHz, DMSO) δ 11.11 (s, 1H), 9.92 (s, 1H), 8.35 (s, 1H), 7.97 (s, 1H), 7.67-7.46 (m, 3H), 7.34-7.18 (m, 2H), 7.04 (d, J=6.9 Hz, 2H), 6.92 (d, J=8.4 Hz, 1H), 5.15-4.99 (m, 1H), 4.54 (d, J=4.5 Hz, 2H), 3.90 (s, 3H), 3.14 (s, 1H), 2.90 (t, J=13.3 Hz, 1H), 2.72-2.56 (m, 2H), 2.41 (s, 1H), 2.25 (s, 2H), 2.02 (d, J=42.5 Hz, 3H), 1.63 (s, 4H).

### Example 99

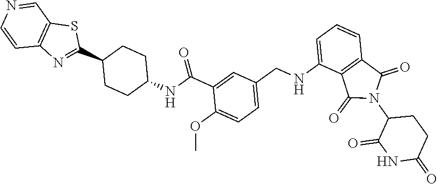 [SMILES: COc1ccc(CNc2cccc3c2C(=O)N(C2CCC(=O)NC2=O)C3=O)cc1C(=O)N[C@H]1CC[C@H](c2nc3ccncc3s2)CC1]

### Preparation Steps

### Step 1

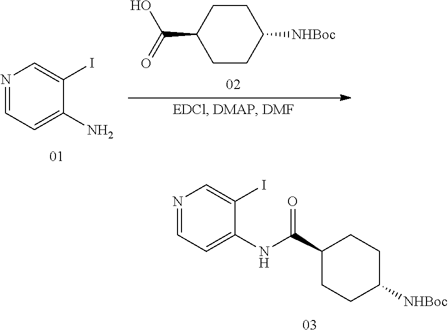 [SMILES: Nc1ccncc1I ; CC(C)(C)OC(=O)N[C@H]1CC[C@H](C(=O)O)CC1 ; CC(C)(C)OC(=O)N[C@H]1CC[C@H](C(=O)Nc2ccncc2I)CC1]

01 (1.00 g, 4.55 mmol), 02 (1.11 g, 4.55 mmol) and 4-dimethylaminopyridine (723 mg, 5.92 mmol) were dissolved in anhydrous N,N-dimethylformamide (15 mL), and 1-(3-dimethylaminopropyl)-3-ethylcarbodiimide hydrochloride (1.13 g, 5.92 mmol) was added with stirring at room temperature. The reaction mixture was stirred at room temperature for 16 h. Water (20 mL) was added to the mixture, followed by filtration to give the target product as a white solid (1.92 g, 94.8% yield). LC-MS: 446.2 [M+H]⁺.

### Step 2

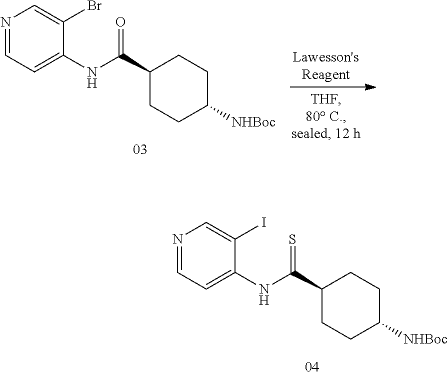 [SMILES: CC(C)(C)OC(=O)N[C@H]1CC[C@H](C(=O)Nc2ccncc2Br)CC1 ; CC(C)(C)OC(=O)N[C@H]1CC[C@H](C(=S)Nc2ccncc2I)CC1]

To a sealed tube were added 03 (830 mg, 1.86 mmol), Lawesson's reagent (1.13 g, 2.79 mmol) and anhydrous tetrahydrofuran (15 mL) at room temperature, and the reaction mixture was stirred at 80° C. for 16 h. Water (100 mL) was added, followed by extraction with ethyl acetate (30 mL×3). The organic phase was washed with saturated brine (50 mL), dried over anhydrous sodium sulfate, and filtered, and the filtrate was concentrated to dryness by rotary evaporation. The residue was purified through a reversed-phase column (C18, 10-100% acetonitrile/water as mobile phase, 0.1% formic acid) to give the target product as a white solid (290 mg, 33.7% yield). LC-MS: 462.3 [M+H]⁺.

### Step 3

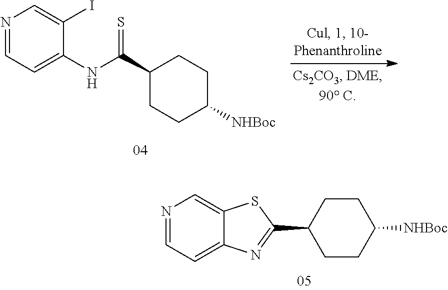 [SMILES: CC(C)(C)OC(=O)N[C@H]1CC[C@H](C(=S)Nc2ccncc2I)CC1 ; CC(C)(C)OC(=O)N[C@H]1CC[C@H](c2nc3ccncc3s2)CC1]

To a sealed tube were added 04 (270 mg, 0.585 mmol), cuprous iodide (11.1 mg, 0.059 mmol), 1,10-phenanthroline (21.1 mg, 0.117 mmol), cesium carbonate (381 mg, 1.17 mmol) and ethylene glycol dimethyl ether (7 mL), and the reaction mixture was stirred at 90° C. for 4 h and filtered. The filtrate was concentrated to dryness by rotary evaporation, and the residue was purified through a flash silica gel column (ethyl acetate:petroleum ether=0-50%) to give the target product as a white solid (100 mg, 51.2% yield). LC-MS: 334.3 [M+H]⁺.

### Step 4

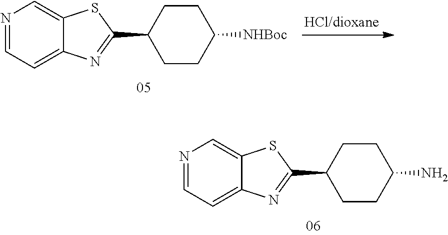 [SMILES: CC(C)(C)OC(=O)N[C@H]1CC[C@H](c2nc3ccncc3s2)CC1 ; N[C@H]1CC[C@H](c2nc3ccncc3s2)CC1]

05 (100 mg, 0.299 mmol) was dissolved in dichloromethane (3 mL) and methanol (3 mL), and a 4 M solution of hydrogen chloride in dioxane (3 mL) was added with stirring at room temperature. The reaction mixture was stirred at room temperature for 4 h and concentrated under reduced pressure to remove the solvent to give the target product as a yellow solid (80.0 mg, crude). LC-MS: 234.1 [M+H]⁺.

### Step 5

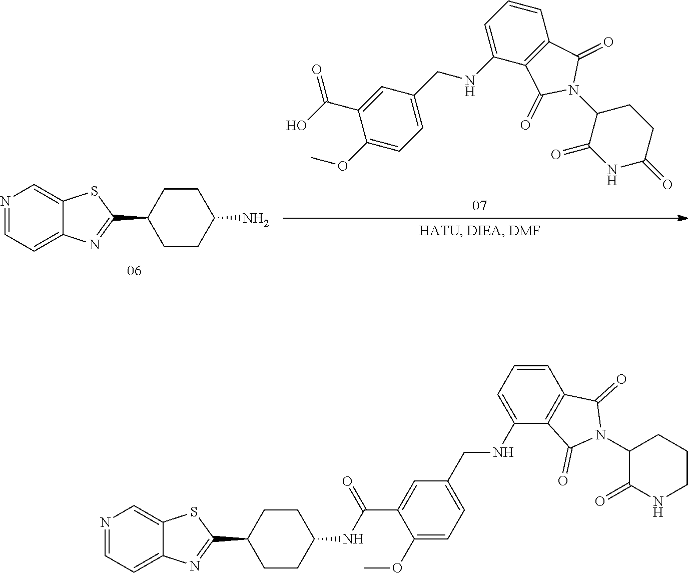 [SMILES: COc1ccc(CNc2cccc3c2C(=O)N(C2CCC(=O)NC2=O)C3=O)cc1C(=O)O ; N[C@H]1CC[C@H](c2nc3ccncc3s2)CC1 ; COc1ccc(CNc2cccc3c2C(=O)N(C2CCCNC2=O)C3=O)cc1C(=O)N[C@H]1CC[C@H](c2nc3ccncc3s2)CC1]

06 (80.0 mg, 0.296 mmol), 07 (129 mg, 0.296 mmol) and N,N-diisopropylethylamine (153 mg, 1.18 mmol) were dissolved in anhydrous N,N-dimethylformamide (4 mL), and (7-azabenzotriazole)-tetramethyluronium hexafluorophosphate (169 mg, 0.444 mmol) was added in batches with stirring at room temperature. The reaction mixture was stirred at room temperature for 1 h. The mixture was purified by preparative reversed-phase liquid chromatography (C18, 5-100% acetonitrile/water as mobile phase, 0.03% trifluoroacetic acid) to separate the target product as a yellowish-green solid (95.75 mg, 49.4% yield). LC-MS: 653.3 [M+H]⁺, ¹H NMR (400 MHz, DMSO-d₆) δ 11.10 (s, 1H), 9.54 (s, 1H), 8.74 (d, J=4.9 Hz, 1H), 8.20 (d, J=6.0 Hz, 1H), 8.03 (d, J=7.7 Hz, 1H), 7.73 (s, 1H), 7.55-7.49 (m, 1H), 7.47 (d, J=8.3 Hz, 1H), 7.27-7.20 (m, 1H), 7.11 (d, J=8.6 Hz, 1H), 7.02 (d, J=7.0 Hz, 1H), 6.98 (d, J=8.7 Hz, 1H), 5.07 (dd, J=12.9, 5.3 Hz, 1H), 4.52 (d, J=3.7 Hz, 2H), 3.91-3.80 (m, 4H), 3.36-3.22 (m, 1H), 2.95-2.82 (m, 1H), 2.64-2.52 (m, 2H), 2.31-2.18 (m, 2H), 2.09-2.01 (m, 3H), 1.82-1.72 (m, 2H), 1.60-1.46 (m, 2H).

### Example 100

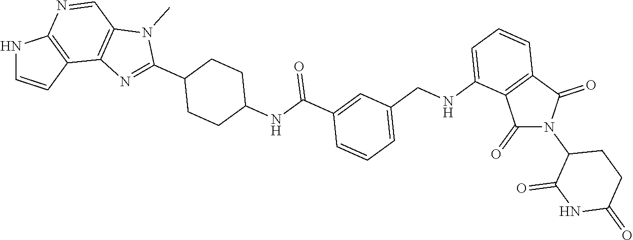 [SMILES: Cn1c(C2CCC(NC(=O)c3cccc(CNc4cccc5c4C(=O)N(C4CCC(=O)NC4=O)C5=O)c3)CC2)nc2c3cc[nH]c3ncc21]

### Preparation Steps

### Step 1

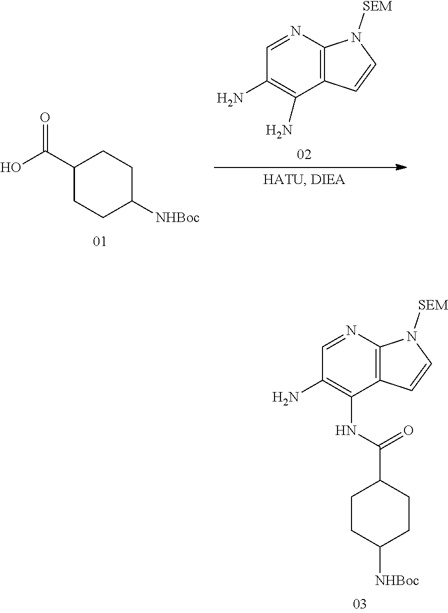 [SMILES: CC(C)(C)OC(=O)NC1CCC(C(=O)O)CC1 ; C[Si](C)(C)CCOCn1ccc2c(N)c(N)cnc21 ; CC(C)(C)OC(=O)NC1CCC(C(=O)Nc2c(N)cnc3c2ccn3COCC[Si](C)(C)C)CC1]

To a 100 mL three-necked flask were added 01 (1 g, 4.11 mmol), 02 (1.14 g, 4.09 mmol), HATU (2.34 g, 6.15 mmol), DIEA (1.59 g, 12.3 mmol) and DMF (20 mL), and the mixture was reacted at room temperature for 16 h. Water (50 mL) was added, followed by extraction with ethyl acetate (30 mL×3). The organic phase was washed with saturated brine (30 mL×2), dried over anhydrous sodium sulfate, and concentrated to dryness by rotary evaporation, and the residue was purified by column chromatography (dichloromethane:methanol=20:1) to give the target product as a brown solid (1.1 g, 53.1% yield). LC-MS: 504.3 [M+H]⁺.

### Step 2

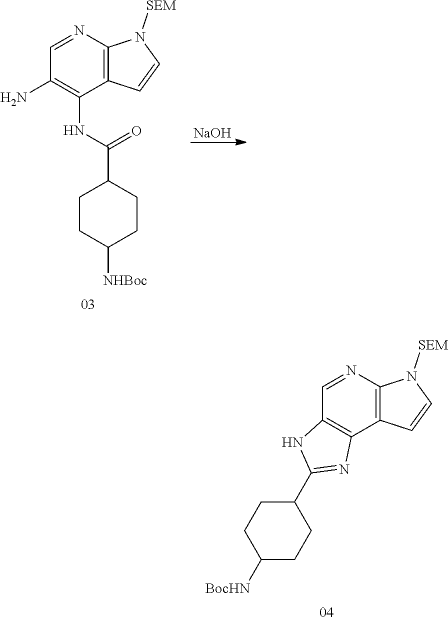

To a 100 mL single-neck round-bottom flask were added 03 (1.1 g, 2.18 mmol), NaOH (261 mg, 6.53 mmol) and absolute ethanol (50 mL), and the mixture was reacted at 80° C. for 16 h. The reaction mixture was concentrated under reduced pressure, and water (50 mL) was added, followed by extraction with ethyl acetate (30 mL×3). The organic phase was washed with saturated brine (30 mL×2), dried over anhydrous sodium sulfate, and concentrated to dryness by rotary evaporation, and the residue was purified by column chromatography to give the target product as a brown solid (700 mg, 66.0% yield). LC-MS: 458.2 [M+H]⁺.

### Step 3

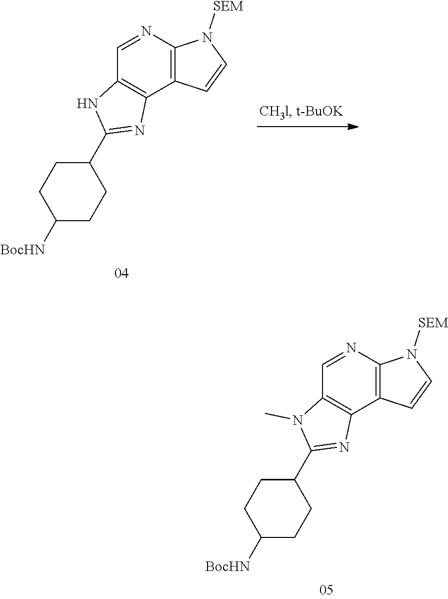 [SMILES: CC(C)(C)OC(=O)NC1CCC(c2nc3c(cnc4c3ccn4COCC[Si](C)(C)C)[nH]2)CC1 ; Cn1c(C2CCC(NC(=O)OC(C)(C)C)CC2)nc2c3ccn(COCC[Si](C)(C)C)c3ncc21]

04 (350 mg, 0.721 mmol) was dissolved in acetonitrile (10 mL) at room temperature, and potassium tert-butoxide (162 mg, 1.44 mmol) was added at 0° C., followed by addition of iodomethane (205 mg, 1.44 mmol). The reaction mixture was heated to room temperature, stirred for 2 h, and filtered, and the filtrate was concentrated to dryness by rotary evaporation. The residue was purified by preparative high performance liquid chromatography to give the target product as a white solid (30 mg, 8.3% yield). LC-MS: 500.3 [M+H]⁺.

### Step 4

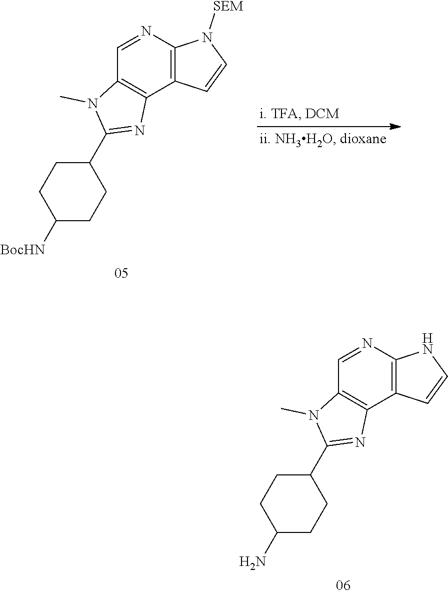 [SMILES: Cn1c(C2CCC(NC(=O)OC(C)(C)C)CC2)nc2c3ccn(COCC[Si](C)(C)C)c3ncc21 ; Cn1c(C2CCC(N)CC2)nc2c3cc[nH]c3ncc21]

To a 100 mL single-neck flask were added 05 (30 mg, 0.06 mmol), trifluoroacetic acid (1 mL) and dichloromethane (5 mL), and the mixture was reacted at room temperature for 16 h and concentrated to give a crude product as a yellow oil, followed by addition of ammonia solution (1 mL) and dioxane (5 mL). The mixture was stirred at 70° C. for 16 h, concentrated, and purified by preparative high performance liquid chromatography to give the target product as a white solid (4.7 mg, 29.1% yield). LC-MS: 270.1 [M+H]⁺.

### Step 5

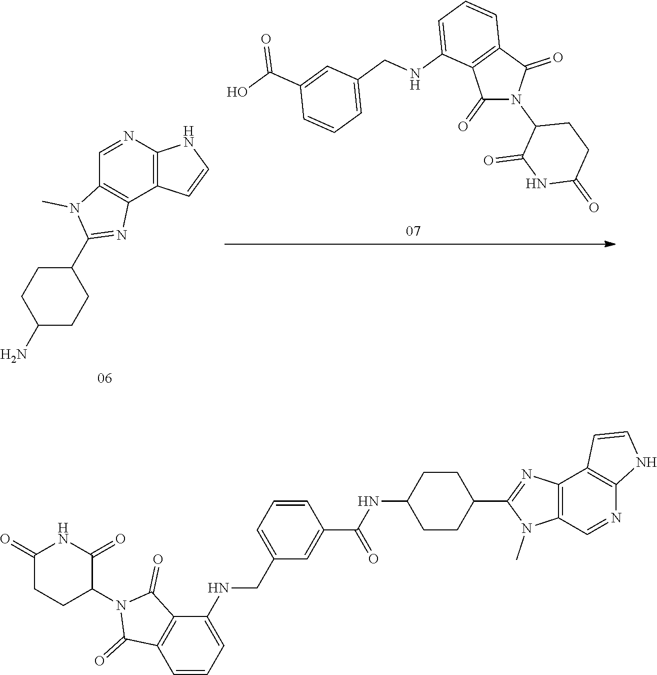 [SMILES: Cn1c(C2CCC(N)CC2)nc2c3cc[nH]c3ncc21 ; O=C1CCC(N2C(=O)c3cccc(NCc4cccc(C(=O)O)c4)c3C2=O)C(=O)N1 ; Cn1c(C2CCC(NC(=O)c3cccc(CNc4cccc5c4C(=O)N(C4CCC(=O)NC4=O)C5=O)c3)CC2)nc2c3cc[nH]c3ncc21]

To a 100 mL single-neck flask were added 06 (4.7 mg, 0.0174 mmol), 07 (7.1 mg, 0.0174 mmol), HATU (10 mg, 0.0263 mmol), DIEA (67 mg, 0.0929 mmol) and DMF (2 mL), and the mixture was reacted at room temperature for 16 h and purified by preparative reversed-phase liquid chromatography to give the target product as a yellowish-green solid (2.60 mg, 22.6% yield).

LC-MS: 659.2 [M+H]⁺.

1H NMR (400 MHz, DMSO-d₆) δ 11.56 (s, 1H), 8.49 (s, 1H), 8.35 (s, 2H), 7.88 (s, 1H), 7.75 (d, J=7.6 Hz, 1H), 7.56-7.48 (m, 2H), 7.43 (t, J=7.6 Hz, 1H), 7.40-7.36 (m, 1H), 7.30 (t, J=6.2 Hz, 1H), 7.04 (d, J=7.0 Hz, 1H), 6.96 (d, J=8.6 Hz, 1H), 6.67-6.63 (m, 1H), 5.32 (t, J=4.5 Hz, 1H), 5.08 (dd, J=12.8, 5.4 Hz, 1H), 4.63 (d, J=6.1 Hz, 2H), 3.91 (s, 3H), 3.02 (t, J=11.6 Hz, 1H), 2.95-2.85 (m, 1H), 2.58 (dd, J=16.4, 10.8 Hz, 2H), 2.03 (s, 3H), 1.98 (d, J=6.8 Hz, 1H), 1.82 (d, J=13.2 Hz, 2H), 1.59 (d, J=11.3 Hz, 2H), 1.46 (d, J=6.9 Hz, 1H).

### Example 101

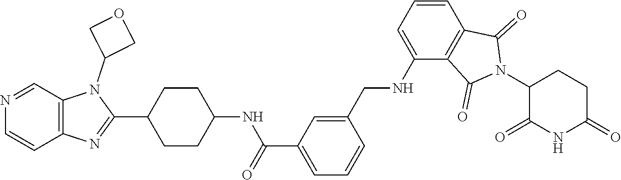 [SMILES: O=C1CCC(N2C(=O)c3cccc(NCc4cccc(C(=O)NC5CCC(c6nc7ccncc7n6C6COC6)CC5)c4)c3C2=O)C(=O)N1]

### Preparation Steps

### Step 1

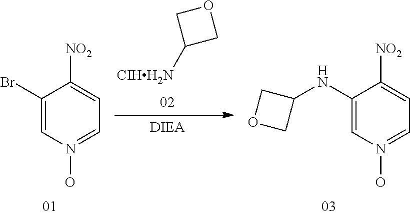

3-bromo-4-nitropyridine-N-oxide (1.10 g, 5.02 mmol), oxetan-3-amine hydrochloride (827 mg, 7.53 mmol) and N,N-diisopropylethylamine (1.29 g, 10.0 mmol) were dissolved in anhydrous N,N-dimethylformamide (10 mL), and the mixture was stirred at 100° C. for 3 h and passed through a reversed-phase column (C18, 10-100% acetonitrile/water as mobile phase, 0.03% trifluoroacetic acid) to separate the target product as a yellow solid (471 mg, 44.4% yield). LC-MS: 212.1 [M+H]⁺.

### Step 2

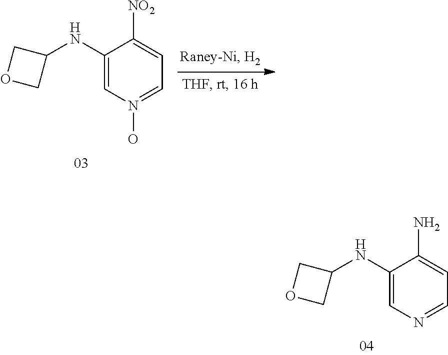

03 (471 mg, 2.23 mmol) was dissolved in tetrahydrofuran (50 mL), and Raney nickel (100 mg) was added. The mixture was purged with hydrogen, stirred in a hydrogen atmosphere at room temperature for 16 h, and filtered, and the filtrate was concentrated to dryness by rotary evaporation to give the target product as a brown solid (290 mg, 78.7% yield). LC-MS: 166.3 [M+H]⁺.

### Step 3

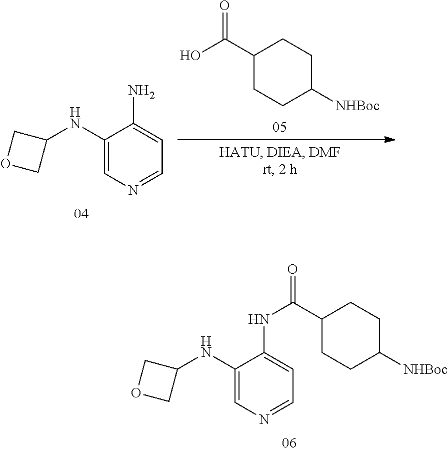 [SMILES: Nc1ccncc1NC1COC1 ; CC(C)(C)OC(=O)NC1CCC(C(=O)O)CC1 ; CC(C)(C)OC(=O)NC1CCC(C(=O)Nc2ccncc2NC2COC2)CC1]

04 (290 mg, 1.76 mmol), 4-(Boc-amino)cyclohexanecarboxylic acid (406 mg, 1.67 mmol) and N,N-diisopropylethylamine (682 mg, 5.28 mmol) were dissolved in anhydrous N,N-dimethylformamide (7 mL), and (7-azabenzotriazole)-tetramethyluronium hexafluorophosphate (1.00 g, 2.64 mmol) was added in batches with stirring at room temperature. The mixture was stirred at room temperature for 2 h and passed through a reversed-phase column (C18, 10-100% acetonitrile/water as mobile phase, 0.03% trifluoroacetic acid) to separate the target product as a white solid (358 mg, 52.2% yield). LC-MS: 391.2 [M+H]⁺.

### Step 4

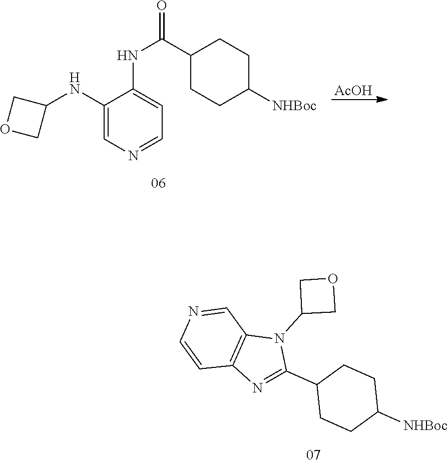 [SMILES: "AcOH" ; CC(C)(C)OC(=O)NC1CCC(C(=O)Nc2ccncc2NC2COC2)CC1 ; CC(C)(C)OC(=O)NC1CCC(c2nc3ccncc3n2C2COC2)CC1]

06 (358 mg, 0.917 mmol) was dissolved in acetic acid (9 mL), and the reaction mixture was stirred at 70° C. for 16 h and concentrated to dryness by rotary evaporation to give the target product as a brown solid (333 mg, crude). LC-MS: 373.2 [M+H]⁺.

### Step 5

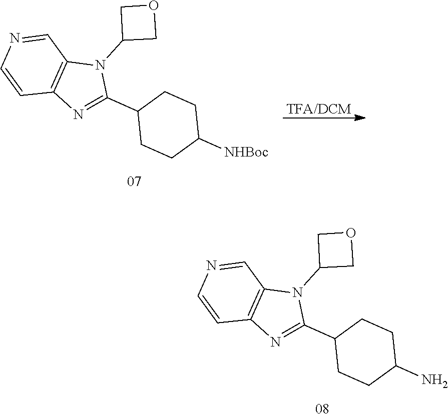 [SMILES: CC(C)(C)OC(=O)NC1CCC(c2nc3ccncc3n2C2COC2)CC1 ; "TFA/DCM" ; NC1CCC(c2nc3ccncc3n2C2COC2)CC1]

07 (140 mg, 0.376 mmol) was dissolved in dichloromethane (2 mL), and trifluoroacetic acid (0.2 mL) was added dropwise with stirring at room temperature. The reaction mixture was stirred at room temperature for 1 h. The mixture was concentrated under reduced pressure to remove the solvent to give the target product as a brown oil (102 mg, crude). LC-MS: 273.3 [M+H]⁺.

### Step 6

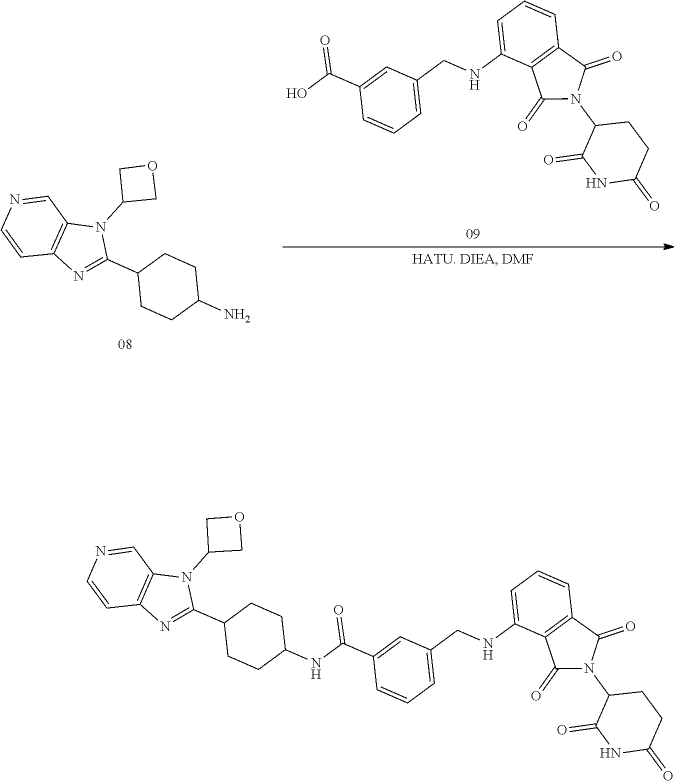 [SMILES: NC1CCC(c2nc3ccncc3n2C2COC2)CC1 ; O=C1CCC(N2C(=O)c3cccc(NCc4cccc(C(=O)O)c4)c3C2=O)C(=O)N1 ; O=C1CCC(N2C(=O)c3cccc(NCc4cccc(C(=O)NC5CCC(c6nc7ccncc7n6C6COC6)CC5)c4)c3C2=O)C(=O)N1]

08 (102 mg, 0.375 mmol), 09 (153 mg, 0.375 mmol) and N,N-diisopropylethylamine (146 mg, 1.13 mmol) were dissolved in anhydrous N,N-dimethylformamide (2 mL), and (7-azabenzotriazole)-tetramethyluronium hexafluorophosphate (214 mg, 0.563 mmol) was added in batches with stirring at room temperature. The reaction mixture was stirred at room temperature for 2 h. The mixture was purified by preparative reversed-phase liquid chromatography (C18, 10-100% acetonitrile/water as mobile phase, 0.1% trifluoroacetic acid) and preparative thin-layer chromatography (dichloromethane/methanol=10/1) to separate the target product as a yellow solid (P1: 5.41 mg, P2: 1.73 mg, 2.9% yield). P1: LC-MS: 662.3 [M+H]⁺, ¹H NMR (400 MHz, DMSO-d₆) δ 11.10 (s, 1H), 9.29 (s, 1H), 8.36 (d, J=5.5 Hz, 1H), 8.33 (d, J=7.8 Hz, 1H), 7.86 (s, 1H), 7.74 (d, J=7.6 Hz, 1H), 7.66 (d, J=5.5 Hz, 1H), 7.54-7.49 (m, 2H), 7.43 (t, J=7.6 Hz, 1H), 7.29 (t, J=5.8 Hz, 1H), 7.04 (d, J=7.0 Hz, 1H), 6.96 (d, J=8.6 Hz, 1H), 5.92-5.84 (m, 1H), 5.17 (t, J=7.6 Hz, 2H), 5.11-5.03 (m, 3H), 4.62 (d, J=6.1 Hz, 2H), 3.87-3.78 (m, 1H), 3.05-2.96 (m, 1H), 2.94-2.82 (m, 1H), 2.64-2.53 (m, 2H), 2.09-2.02 (m, 1H), 2.01-1.91 (m, 4H), 1.79-1.67 (m, 2H), 1.61-1.50 (m, 2H).

### Example 102

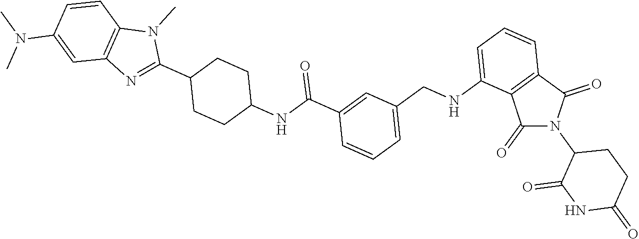 [SMILES: CN(C)c1ccc2c(c1)nc(C1CCC(NC(=O)c3cccc(CNc4cccc5c4C(=O)N(C4CCC(=O)NC4=O)C5=O)c3)CC1)n2C]

### Preparation Steps

### Step 1

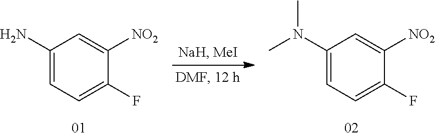

60% sodium hydride (2.05 g, 51.2 mmol) was mixed in anhydrous N,N-dimethylformamide (50 mL), and 4-fluoro-3-nitroaniline (2 g, 12.8 mmol) was added with stirring at 0° C. The reaction mixture was stirred at 0° C. for 0.5 h, then iodomethane (9.09 g, 64 mmol) was added, and the reaction mixture was stirred at room temperature for 12 h. Ice water (50 mL) was added to the mixture, followed by extraction with ethyl acetate (30 mL×3). The organic phase was washed with saturated brine (30 mL×2), dried, and filtered, and the filtrate was concentrated to dryness by rotary evaporation. The residue was passed through a flash silica gel column (petroleum ether:ethyl acetate=3:1) to separate the target product as a red solid (1.1 g, 47% yield). LC-MS: 185.3 [M+H]⁺.

### Step 2

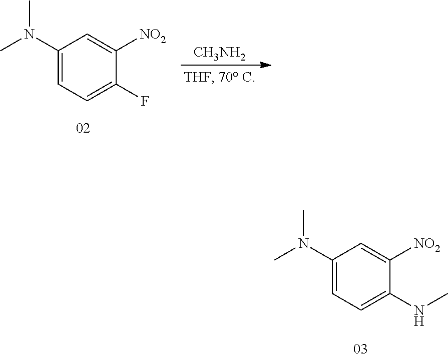

02 (500 mg, 2.72 mmol) was dissolved in tetrahydrofuran (20 mL), and a solution of methylamine in ethanol (1.61 g, 13.56 mmol) was added at room temperature. The reaction mixture was stirred at 70° C. for 36 h, cooled to room temperature, and then concentrated. Water (20 mL) was added, followed by extraction with ethyl acetate (20 mL×2). The organic phase was washed with saturated brine (20 mL×3), dried over anhydrous sodium sulfate, and filtered, and the filtrate was concentrated to dryness by rotary evaporation. The residue was passed through a flash silica gel column (petroleum ether:ethyl acetate=1:1) to separate the target product as a black solid (500 mg, 94% yield). LC-MS: 196.1 [M+H]⁺.

### Step 3

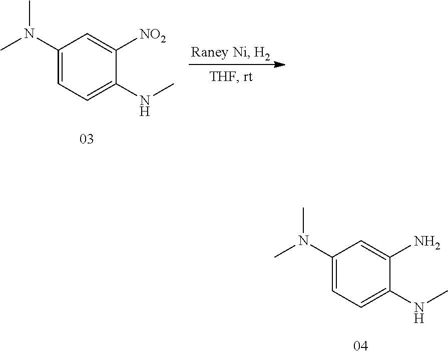

03 (250 mg, 1.28 mmol) was dissolved in tetrahydrofuran (20 mL) at room temperature, and Raney nickel (200 mg) was added. The reaction mixture was purged with hydrogen three times, stirred in a hydrogen atmosphere at room temperature for 2 h, and filtered, and the filtrate was concentrated to dryness by rotary evaporation to give the target product as a red solid (150 mg, 71% yield). LC-MS: 166.2 [M+H]⁺.

### Step 4

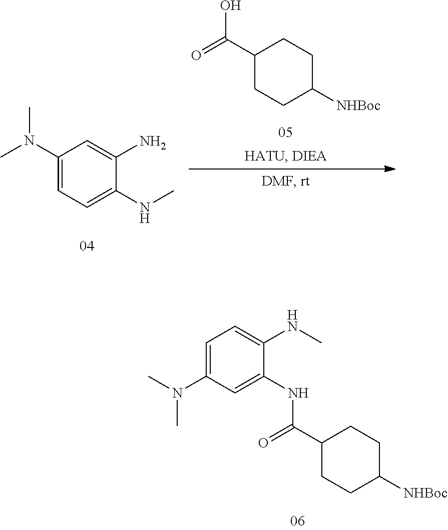

04 (150 mg, 0.907 mmol) was dissolved in anhydrous N,N-dimethylformamide (20 mL), and (7-azabenzotriazole)-tetramethyluronium hexafluorophosphate (448 mg, 1.18 mmol), N,N-diisopropylethylamine (351 mg, 2.72 mmol) and 05 (190 mg, 0.781 mmol) were added successively with stirring at room temperature. The reaction mixture was stirred at room temperature for 2 h. Ice water (20 mL) was added, followed by extraction with ethyl acetate (20 mL×2). The organic phase was washed with saturated brine (20 mL×3), dried, and filtered, and the filtrate was concentrated to dryness by rotary evaporation. The residue was passed through a flash silica gel column (dichloromethane:methanol=10:1) to separate the target product as a white solid (100 mg, 33% yield). LC-MS: 391.3 [M+H]⁺.

### Step 5

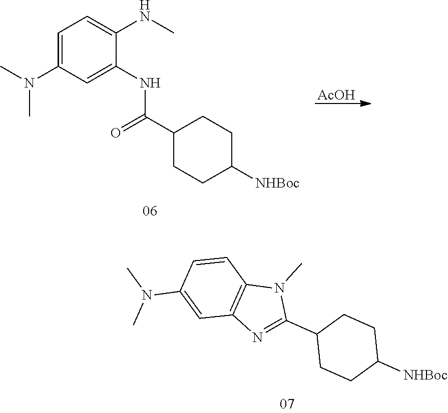 [SMILES: "AcOH" ; CNc1ccc(N(C)C)cc1NC(=O)C1CCC(NC(=O)OC(C)(C)C)CC1 ; CN(C)c1ccc2c(c1)nc(C1CCC(NC(=O)OC(C)(C)C)CC1)n2C]

06 (100 mg, 0.256 mmol) was dissolved in acetic acid (1 mL), and the reaction mixture was stirred at 70° C. for 2.5 h concentrated under reduced pressure to remove the solvent, diluted with ethyl acetate (20 mL), washed with saturated aqueous sodium bicarbonate solution (20 mL×3) and water (20 mL), dried, and filtered. The filtrate was concentrated to dryness by rotary evaporation. The residue was passed through a flash silica gel column (dichloromethane:methanol=10:1) to separate the target product as a yellow solid (70 mg, 73% yield). LC-MS: 373.3 [M+H]⁺.

### Step 6

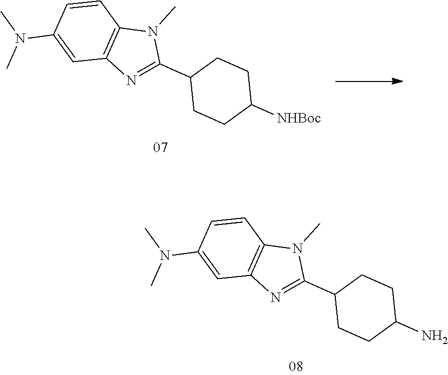 [SMILES: CN(C)c1ccc2c(c1)nc(C1CCC(NC(=O)OC(C)(C)C)CC1)n2C ; CN(C)c1ccc2c(c1)nc(C1CCC(N)CC1)n2C]

07 (35.0 mg, 0.09 mmol) was dissolved in dichloromethane (2 mL), and a 4 M solution of hydrogen chloride in dioxane (0.5 mL) was added with stirring at room temperature. The reaction mixture was stirred at room temperature for 1 h and concentrated under reduced pressure to remove the solvent to give the target product as a yellow solid (22.0 mg, crude). LC-MS: 273.2 [M+H]⁺.

### Step 7

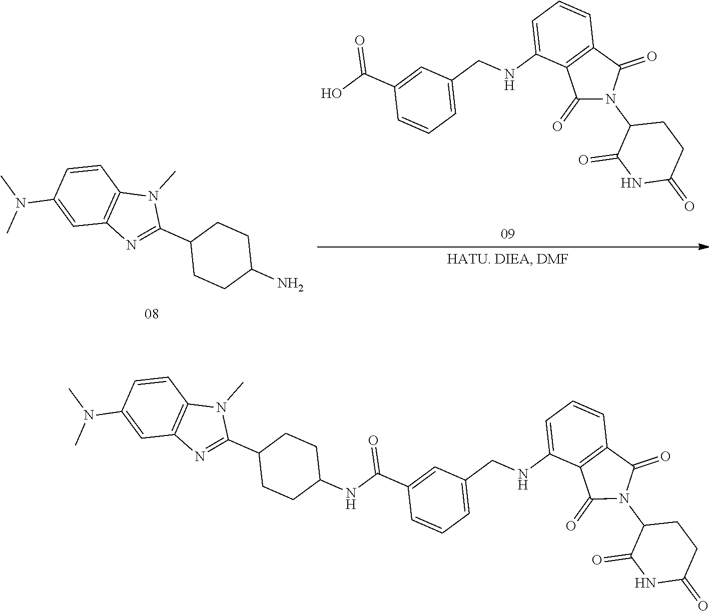 [SMILES: CN(C)c1ccc2c(c1)nc(C1CCC(N)CC1)n2C ; O=C1CCC(N2C(=O)c3cccc(NCc4cccc(C(=O)O)c4)c3C2=O)C(=O)N1 ; CN(C)c1ccc2c(c1)nc(C1CCC(NC(=O)c3cccc(CNc4cccc5c4C(=O)N(C4CCC(=O)NC4=O)C5=O)c3)CC1)n2C]

08 (22 mg, 0.081 mmol) and (7-azabenzotriazole)-tetramethyluronium hexafluorophosphate (40 mg, 0.105 mmol) were dissolved in anhydrous N,N-dimethylformamide (1 mL) at room temperature, and N,N-diisopropylethylamine (52 mg, 0.402 mmol) and 09 (32 mg, 0.078 mmol) were added with stirring at room temperature. The reaction mixture was stirred at room temperature for 1 h. The mixture was purified by preparative chromatography (C18, 5-100% acetonitrile/water as mobile phase, 0.1% formic acid) to separate the target product as a yellow solid (26.59 mg, 51% yield). LC-MS: 662.4 [M+H]⁺, ¹H NMR (400 MHz, DMSO-d₆) δ 11.10 (s, 1H), 8.30 (d, J=7.9 Hz, 6H), 7.89-7.69 (m, 6H), 7.62-7.49 (m, 5H), 7.42 (t, J=7.6 Hz, 4H), 7.38-7.23 (m, 6H), 7.03 (d, J=7.0 Hz, 3H), 6.96 (d, J=8.6 Hz, 3H), 6.90 (d, J=2.2 Hz, 3H), 6.79 (dd, J=8.8, 2.2 Hz, 3H), 5.08 (dd, J=12.8, 5.4 Hz, 3H), 4.62 (d, J=6.0 Hz, 6H), 3.84 (d, J=7.6 Hz, 6H), 3.75-3.65 (m, 11H), 2.87 (d, J=14.6 Hz, 40H), 2.61-2.44 (m, 81H), 2.40-1.86 (m, 22H), 1.86-1.48 (m, 14H).

### Example 103

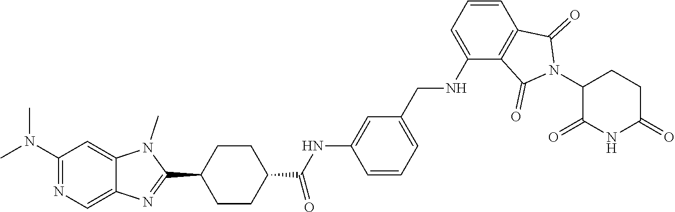 [SMILES: CN(C)c1cc2c(cn1)nc([C@H]1CC[C@H](C(=O)Nc3cccc(CNc4cccc5c4C(=O)N(C4CCC(=O)NC4=O)C5=O)c3)CC1)n2C]

### Preparation Steps

### Step 1

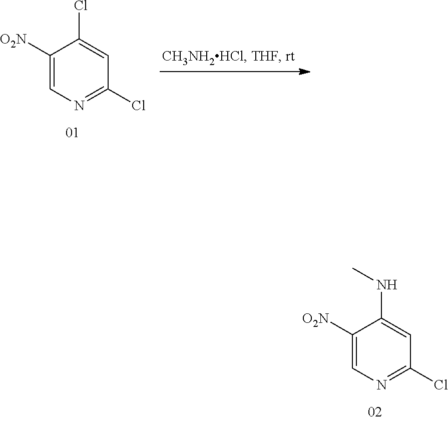

To a 250 mL single-neck flask were added 01 (10 g, 51.8 mmol), methylamine hydrochloride (4.2 g, 62.2 mmol), triethylamine (21.0 mg, 207 mmol) and tetrahydrofuran (100 mL), and the mixture was reacted at 30° C. for 3 h, quenched with water, and extracted with ethyl acetate (50 mL×3). The organic phase was washed with saturated brine (50 mL×2), dried over anhydrous sodium sulfate, and concentrated to dryness by rotary evaporation to give a yellow solid (9.80 g, crude), which was directly used in the next step without purification. LC-MS: 188.2 [M+H]⁺.

### Step 2

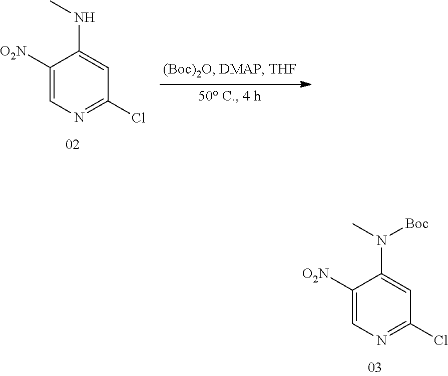 [SMILES: CNc1cc(Cl)ncc1[N+](=O)[O-] ; CN(C(=O)OC(C)(C)C)c1cc(Cl)ncc1[N+](=O)[O-]]

To a 250 mL single-neck flask were added 02 (9.8 g, 52.2 mmol), di-tert-butyl dicarbonate (11.4 g, 52.2 mmol), 4-dimethylaminopyridine (1.28 g, 10.4 mmol) and tetrahydrofuran (50 mL), and the mixture was reacted at 50° C. for 1 h, quenched with water, and extracted with ethyl acetate (50 mL×3). The organic phase was washed with saturated brine (50 mL×2), dried over anhydrous sodium sulfate, and concentrated to dryness by rotary evaporation, and the residue was purified by column chromatography (ethyl acetate:petroleum ether=1:2) to give a yellow solid (4.96 g, 33.1% yield). LC-MS: 288.2 [M+H]⁺.

### Step 3

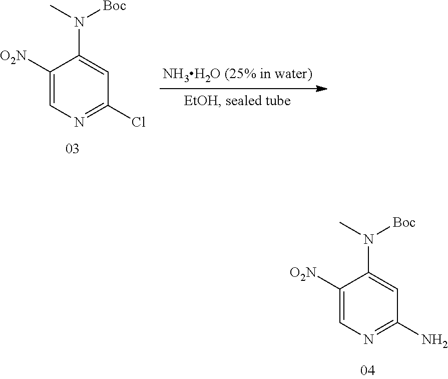 [SMILES: CN(C(=O)OC(C)(C)C)c1cc(Cl)ncc1[N+](=O)[O-] ; CN(C(=O)OC(C)(C)C)c1cc(N)ncc1[N+](=O)[O-]]

To a 100 mL closed container were added 03 (4.96 g, 17.30 mmol), ammonia solution (30 mL) and ethanol (30 mL), and the mixture was reacted at 80° C. for 12 h, quenched with water, and extracted with ethyl acetate (50 mL×3). The organic phase was washed with saturated brine (50 mL×2), dried over anhydrous sodium sulfate, and concentrated to dryness by rotary evaporation, and the residue was purified by column chromatography (ethyl acetate:petroleum ether=1:2) to give a yellow solid (2.06 g, 44.5% yield). LC-MS: 269.3 [M+H]⁺.

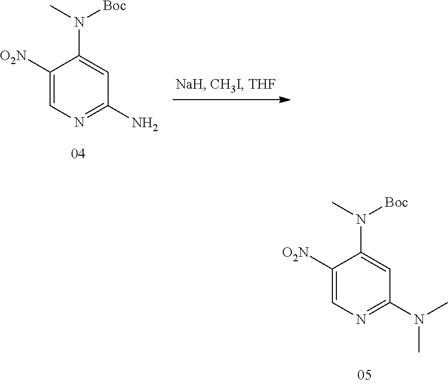 [SMILES: CN(C(=O)OC(C)(C)C)c1cc(N)ncc1[N+](=O)[O-] ; CN(C)c1cc(N(C)C(=O)OC(C)(C)C)c([N+](=O)[O-])cn1]

To a 100 mL single-neck flask were added 04 (2.99 g, 11.145 mmol) and tetrahydrofuran (30 mL), and sodium hydride (1.34 g, 55.7 mmol) was added at 0° C. After the mixture was cooled in an ice bath for 20 min, iodomethane (7.91 g, 55.7 mmol) was added, and the mixture was reacted at room temperature for 12 h, quenched with water, and extracted with ethyl acetate (50 mL×3). The organic phase was washed with saturated brine (50 mL×2), dried over anhydrous sodium sulfate, and concentrated to dryness by rotary evaporation, and the residue was purified by column chromatography (ethyl acetate:petroleum ether=1:2) to give a yellow solid (3.12 g, 94% yield). LC-MS: 297.2 [M+H]⁺.

### Step 5

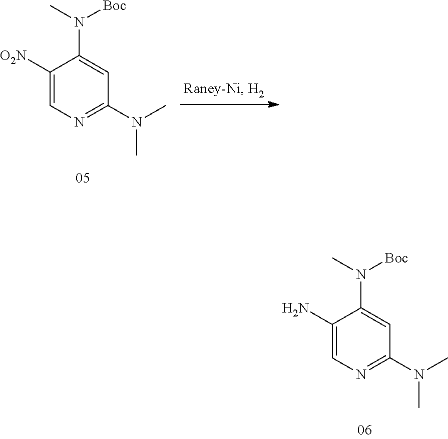 [SMILES: CN(C)c1cc(N(C)C(=O)OC(C)(C)C)c([N+](=O)[O-])cn1 ; CN(C)c1cc(N(C)C(=O)OC(C)(C)C)c(N)cn1]

To a 100 mL single-neck flask were added 05 (1 g, 3.375 mmol), Raney nickel and MeOH (15 mL), and the mixture was reacted at room temperature for 12 h and filtered to give a reddish-brown solid (1 g, 100% yield). LC-MS: 267.3 [M+H]⁺.

### Step 6

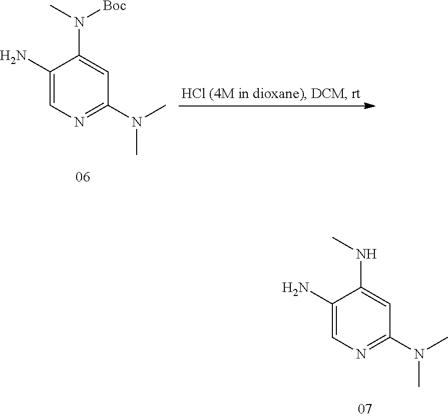 [SMILES: CN(C)c1cc(N(C)C(=O)OC(C)(C)C)c(N)cn1 ; CNc1cc(N(C)C)ncc1N]

A solution of hydrochloric acid in dioxane (4 M, 2.5 mL) was added to a solution of 06 (1 g, 3.759 mmol) in dichloromethane (10 mL) at room temperature, and the mixture was reacted at room temperature for 12 h and concentrated to dryness by rotary evaporation to give a purple solid (1 g, 100% yield). LC-MS: 167.3 [M+H]⁺.

### Step 7

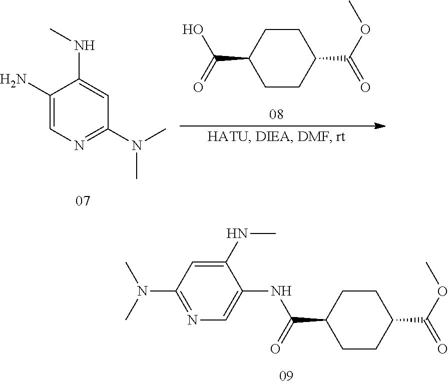 [SMILES: CNc1cc(N(C)C)ncc1N ; COC(=O)[C@H]1CC[C@H](C(=O)O)CC1 ; CNc1cc(N(C)C)ncc1NC(=O)[C@H]1CC[C@H](C(=O)OC)CC1]

To a 100 mL single-neck flask were added 07 (500 mg, 3.01 mmol), 08 (504 mg, 2.71 mmol), 2-(7-azabenzotriazol)-N,N,N,N-tetramethyluronium hexafluorophosphate (1.72 g, 4.52 mmol), N,N-diisopropylethylamine (1.17 g, 9.03 mmol) and N,N-dimethylformamide (5 mL), and the mixture was reacted at room temperature for 12 h and purified through a reversed-phase column (acetonitrile/water=56%, 0.3% FA) to give a brown oily liquid (1.11 g, 100% yield). LC-MS: 335.3 [M+H]⁺.

### Step 8

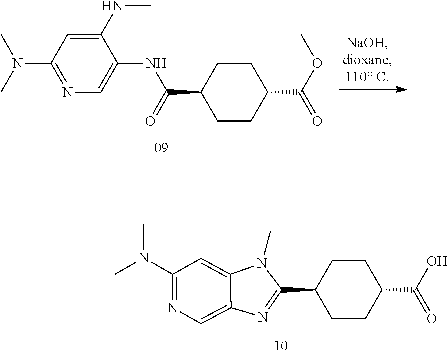 [SMILES: CNc1cc(N(C)C)ncc1NC(=O)[C@H]1CC[C@H](C(=O)OC)CC1 ; CN(C)c1cc2c(cn1)nc([C@H]1CC[C@H](C(=O)O)CC1)n2C]

To a 100 mL single-neck flask were added 09 (345 mg, 1.03 mmol), dioxane (10 mL) and sodium hydroxide solution (4 M, 2 mL), and the mixture was reacted at 110° C. for 2 h and purified through a reversed-phase column (acetonitrile/water=40%, 0.3% FA) to give a brown oily liquid (47 mg, 15% yield). LC-MS: 303.3 [M+H]⁺.

### Step 9

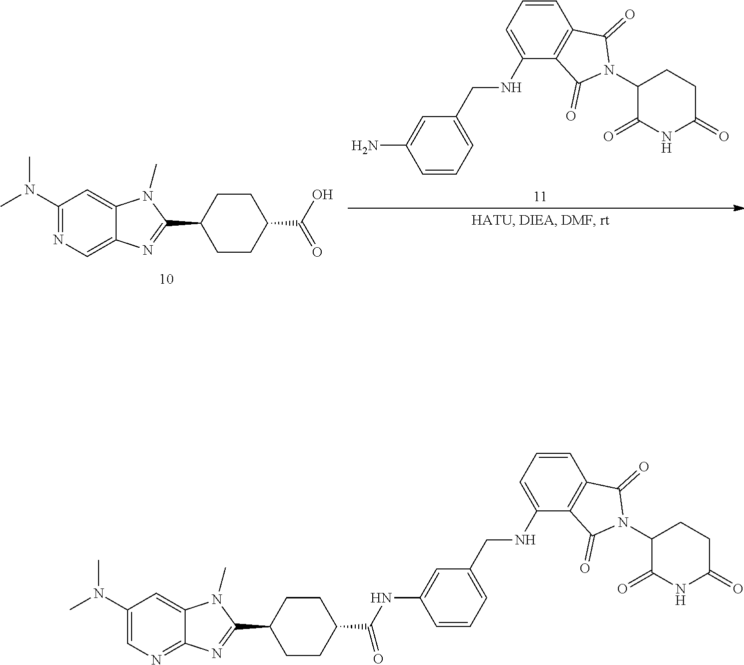 [SMILES: CN(C)c1cc2c(cn1)nc([C@H]1CC[C@H](C(=O)O)CC1)n2C ; Nc1cccc(CNc2cccc3c2C(=O)N(C2CCC(=O)NC2=O)C3=O)c1 ; CN(C)c1cnc2nc([C@H]3CC[C@H](C(=O)Nc4cccc(CNc5cccc6c5C(=O)N(C5CCC(=O)NC5=O)C6=O)c4)CC3)n(C)c2c1]

To a 50 mL single-neck flask were added 10 (30 mg, 0.099 mmol), intermediate 11 (33.71 mg, 0.0891 mmol), 2-(7-azabenzotriazol)-N,N,N,N-tetramethyluronium hexafluorophosphate (56.46 mg, 0.1485 mmol), N,N-diisopropylethylamine (38.4 mg, 0.297 mmol) and N,N-dimethylformamide (1 mL), and the reaction mixture was reacted at room temperature for 12 h and purified by preparative high performance liquid chromatography to give a yellow solid (30.36 mg, 46.3% yield). LC-MS: 663.4 [M+H]⁺.

¹H NMR (400 MHz, DMSO-d₆) δ 11.11 (s, 1H), 9.92 (s, 1H), 8.53 (s, 1H), 7.60 (d, J=7.7 Hz, 1H), 7.57-7.48 (m, 2H), 7.30-7.18 (m, 2H), 7.08 (d, J=5.8 Hz, 1H), 7.04 (d, J=7.1 Hz, 2H), 6.92 (d, J=8.6 Hz, 1H), 5.08 (dd, J=12.7, 5.1 Hz, 1H), 4.54 (d, J=5.9 Hz, 2H), 3.78 (d, J=12.3 Hz, 4H), 3.19 (s, 6H), 3.08 (s, 1H), 2.88 (d, J=12.1 Hz, 1H), 2.57 (d, J=7.1 Hz, 1H), 2.43 (s, 1H), 2.08 (d, J=12.3 Hz, 3H), 1.96 (d, J=12.3 Hz, 2H), 1.73-1.53 (m, 4H).

### Example 104

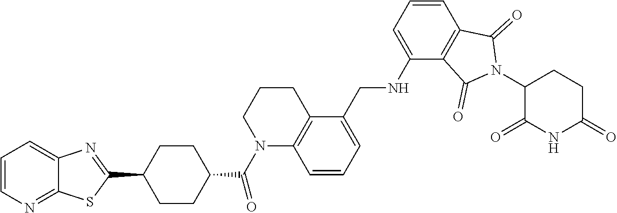 [SMILES: O=C1CCC(N2C(=O)c3cccc(NCc4cccc5c4CCCN5C(=O)[C@H]4CC[C@H](c5nc6cccnc6s5)CC4)c3C2=O)C(=O)N1]

### Preparation Steps

### Step 1

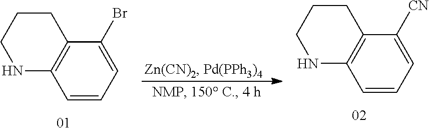 [SMILES: Brc1cccc2c1CCCN2 ; N#Cc1cccc2c1CCCN2]

To a sealed tube were added 01 (550 mg, 2.59 mmol), zinc cyanide (304 mg, 2.59 mmol), tetrakis(triphenylphosphine)palladium (299 mg, 0.259 mmol) and anhydrous N-methylpyrrolidone (20 mL), and the above mixture was stirred at 150° C. for 4 h. Water (100 mL) was added, followed by extraction with ethyl acetate (20 mL×3). The organic phase was washed with saturated brine (50 mL), dried over anhydrous sodium sulfate, and filtered, and the filtrate was concentrated to dryness by rotary evaporation. The residue was purified through a flash silica gel column (petroleum ether:ethyl acetate=2:1) to give the target product as a white solid (357 mg, 87.0% yield). LC-MS: 159.3 [M+H]⁺.

### Step 2

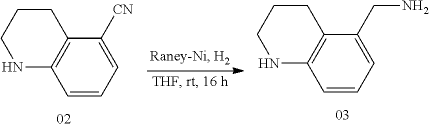 [SMILES: N#Cc1cccc2c1CCCN2 ; NCc1cccc2c1CCCN2]

02 (357 mg, 2.26 mmol) was dissolved in tetrahydrofuran (5 mL) at room temperature, and then Raney nickel (100 mg) was added. The reaction mixture was stirred in a hydrogen atmosphere at room temperature for 16 h and filtered, and the filtrate was concentrated to dryness by rotary evaporation to give the target product as a colorless oil (300 mg, 81.9% yield). LC-MS: 162.4 [M+H]⁺.

### Step 3

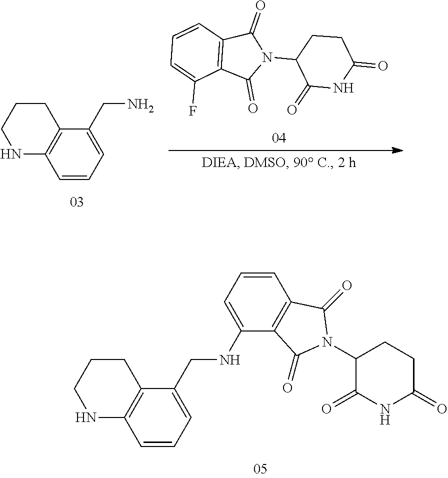 [SMILES: NCc1cccc2c1CCCN2 ; O=C1CCC(N2C(=O)c3cccc(F)c3C2=O)C(=O)N1 ; O=C1CCC(N2C(=O)c3cccc(NCc4cccc5c4CCCN5)c3C2=O)C(=O)N1]

03 (300 mg, 1.85 mmol), 04 (358 mg, 1.29 mmol) and N,N-diisopropylethylamine (717 mg, 5.55 mmol) were dissolved in anhydrous dimethyl sulfoxide (10 mL) at room temperature. The reaction mixture was stirred at 90° C. for 2 h. The mixture was cooled to room temperature, and water (80 mL) was added, followed by extraction with ethyl acetate (20 mL×3). The organic phase was washed with saturated brine (50 mL), dried over anhydrous sodium sulfate, and filtered, and the filtrate was concentrated to dryness by rotary evaporation. The residue was purified through a flash silica gel column (petroleum ether:ethyl acetate=1:1) to give the target product as a yellow solid (317 mg, 41.0% yield). LC-MS: 419.3 [M+H]⁺.

### Step 4

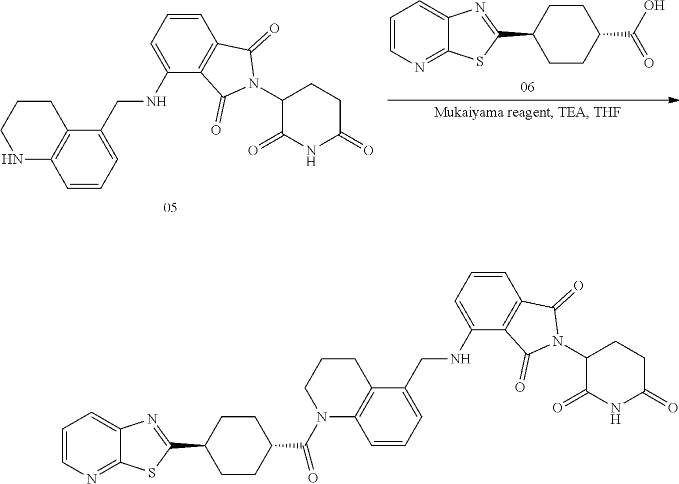 [SMILES: O=C1CCC(N2C(=O)c3cccc(NCc4cccc5c4CCCN5)c3C2=O)C(=O)N1 ; O=C(O)[C@H]1CC[C@H](c2nc3cccnc3s2)CC1 ; O=C1CCC(N2C(=O)c3cccc(NCc4cccc5c4CCCN5C(=O)[C@H]4CC[C@H](c5nc6cccnc6s5)CC4)c3C2=O)C(=O)N1]

To a sealed tube were added at room temperature 05 (100 mg, 0.239 mmol), 06 (62.7 mg, 0.239 mmol), triethylamine (58.0 mg, 0.574 mmol), 2-chloro-1-methylpyridinium iodide (73.3 mg, 0.287 mmol) and anhydrous tetrahydrofuran (5 mL), and the reaction mixture was stirred at 70° C. for 16 h. The mixture was purified by preparative chromatography (C18, 10-100% acetonitrile/water as mobile phase, 0.1% formic acid) to separate the target product as a green solid (42.54 mg, 26.9% yield). LC-MS: 663.4 [M+H]⁺, ¹H NMR (400 MHz, DMSO-d₆) δ 11.09 (s, 1H), 8.56 (dd, J=4.6, 1.4 Hz, 1H), 8.31 (dd, J=8.2, 1.2 Hz, 1H), 7.57-7.51 (m, 2H), 7.26 (s, 1H), 7.17 (t, J=7.4 Hz, 1H), 7.10-7.00 (m, 3H), 6.96 (d, J=8.6 Hz, 1H), 5.08 (dd, J=12.8, 5.3 Hz, 1H), 4.55 (d, J=5.1 Hz, 2H), 3.74 (t, J=5.2 Hz, 2H), 3.17 (t, J=11.7 Hz, 1H), 2.96-2.84 (m, 2H), 2.78 (t, J=6.7 Hz, 2H), 2.62-2.53 (m, 2H), 2.17 (d, J=10.5 Hz, 2H), 2.09-2.02 (m, 1H), 1.98-1.84 (m, 4H), 1.71-1.49 (m, 4H).

### Example 105

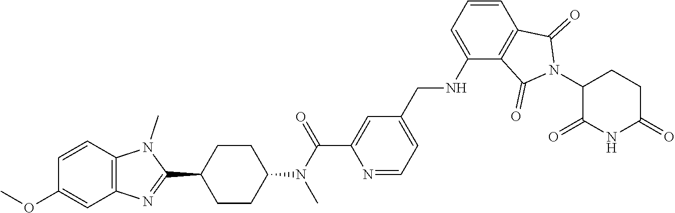 [SMILES: COc1ccc2c(c1)nc([C@H]1CC[C@H](N(C)C(=O)c3cc(CNc4cccc5c4C(=O)N(C4CCC(=O)NC4=O)C5=O)ccn3)CC1)n2C]

### Preparation Steps

### Step 1

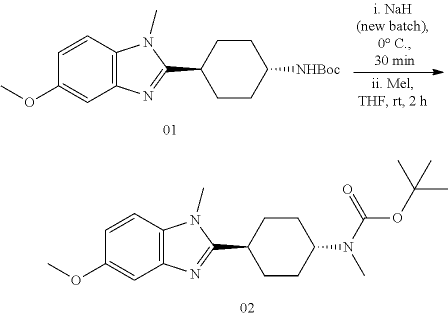 [SMILES: COc1ccc2c(c1)nc([C@H]1CC[C@H](NC(=O)OC(C)(C)C)CC1)n2C ; COc1ccc2c(c1)nc([C@H]1CC[C@H](N(C)C(=O)OC(C)(C)C)CC1)n2C]

01 (25 mg, 0.0695 mmol) was dissolved in N,N-dimethylformamide (5 mL) at room temperature, and sodium hydride (60%) (14 mg, 0.348 mmol) was added at 0° C. The reaction mixture was stirred at 0° C. for 1 h, then iodomethane (13 mg, 0.0904 mmol) was added, and the reaction mixture was stirred at room temperature for 16 h, quenched with ammonium chloride (10 mL), and extracted with ethyl acetate (10 mL×3). The organic phase was washed with saturated brine, dried over anhydrous sodium sulfate, and concentrated to dryness by rotary evaporation to give the target product as a yellow oily liquid (24 mg, 82.3% yield). LC-MS: 374.2 [M+H]⁺.

### Step 2

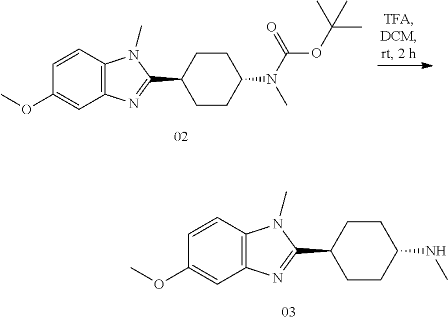

02 (24 mg, 0.0643 mmol) was dissolved in dichloromethane (2 mL) at room temperature, and trifluoroacetic acid (0.3 mL) was added at room temperature. The reaction mixture was stirred at room temperature for 2 h and concentrated to dryness by rotary evaporation to give the target product as a yellow oily liquid (21 mg, crude). LC-MS: 274.2 [M+H]⁺.

### Step 3

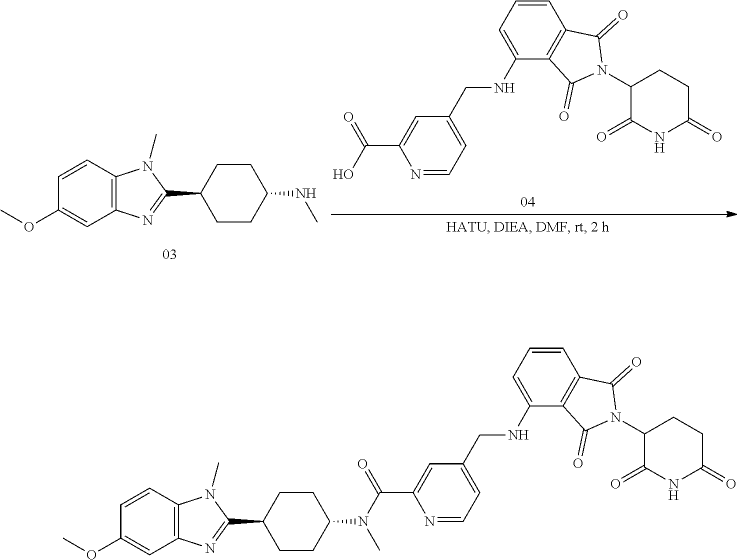

03 (21 mg, 0.0643), 04 (34 mg, 0.0834 mmol) and N,N-diisopropylethylamine (26 mg, 0.209 mmol) were dissolved in N,N-dimethylformamide (1 mL) at room temperature, and (7-azabenzotriazole)-tetramethyluronium hexafluorophosphate (39 mg, 0.104 mmol) was added with stirring at room temperature. The reaction mixture was stirred at room temperature for 2 h. The mixture was purified by preparative chromatography to separate the target product as a yellow solid (5.37 mg, 11.7% yield). LC-MS: 664.4.

¹H NMR (400 MHz, DMSO-d₆) δ 11.11 (s, 1H), 8.53 (dd, J=10.8, 5.0 Hz, 1H), 8.38 (s, 2H), 7.53-7.48 (m, 1H), 7.42 (dd, J=21.0, 12.9 Hz, 3H), 7.34 (d, J=9.0 Hz, 1H), 7.13-6.94 (m, 2H), 6.93-6.88 (m, 1H), 6.83-6.78 (m, 1H), 5.08 (dd, J=12.7, 5.4 Hz, 1H), 4.69 (d, J=6.2 Hz, 2H), 2.88 (d, J=23.8 Hz, 4H), 2.57 (d, J=30.1 Hz, 2H), 2.10-1.96 (m, 3H), 1.81 (dd, J=34.7, 14.6 Hz, 6H), 1.33 (d, J=15.1 Hz, 2H).

### Example 106

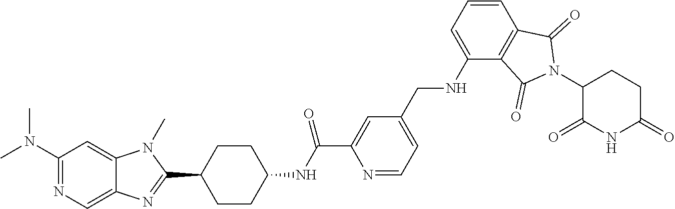 [SMILES: CN(C)c1cc2c(cn1)nc([C@H]1CC[C@H](NC(=O)c3cc(CNc4cccc5c4C(=O)N(C4CCC(=O)NC4=O)C5=O)ccn3)CC1)n2C]

### Preparation Steps

### Step 1

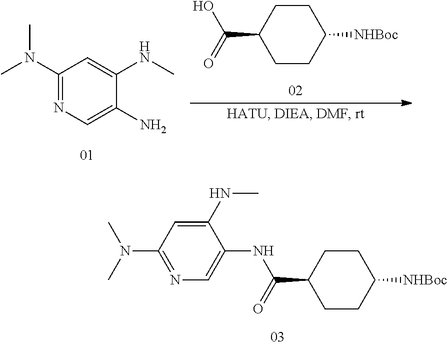

To a 100 mL single-neck flask were added 01 (500 mg, 3.01 mmol), 02 (659.1 mg, 2.709 mmol), 2-(7-azabenzotriazol)-N,N,N,N-tetramethyluronium hexafluorophosphate (1716.73 mg, 4.515 mmol), N,N-diisopropylethylamine (1167.04 mg, 9.03 mmol) and N,N-dimethylformamide (5 mL), and the mixture was reacted at room temperature for 12 h and purified through a reversed-phase column (acetonitrile/water=60%, 0.3% FA) to give a purple solid (500 mg, 42.5% yield).

LC-MS: 392.4 [M+H]⁺.

### Step 2

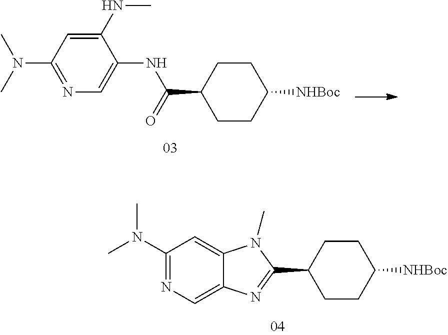 [SMILES: CNc1cc(N(C)C)ncc1NC(=O)[C@H]1CC[C@H](NC(=O)OC(C)(C)C)CC1 ; CN(C)c1cc2c(cn1)nc([C@H]1CC[C@H](NC(=O)OC(C)(C)C)CC1)n2C]

To a 100 mL single-neck flask were added 03 (430 mg, 1.01 mmol), dioxane (10 mL) and sodium hydroxide solution (4 M, 2 mL), and the mixture was reacted at 110° C. for 2 h and purified (acetonitrile/water=55%, 0.3% FA) to give a brown oily liquid (169 mg, 44.9% yield).

LC-MS: 374.4 [M+H]⁺.

### Step 3

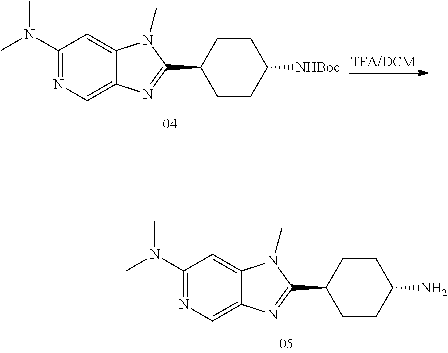 [SMILES: CN(C)c1cc2c(cn1)nc([C@H]1CC[C@H](NC(=O)OC(C)(C)C)CC1)n2C ; CN(C)c1cc2c(cn1)nc([C@H]1CC[C@H](N)CC1)n2C]

To a 100 mL single-neck flask were added 04 (80 mg, 0.214 mmol), trifluoroacetic acid (3 mL) and dichloromethane (2 mL), and the mixture was reacted at room temperature for 12 h and concentrated to dryness by rotary evaporation to give a brown oily liquid (80 mg, 100% yield).

LC-MS: 274.2 [M+H]⁺.

### Step 4

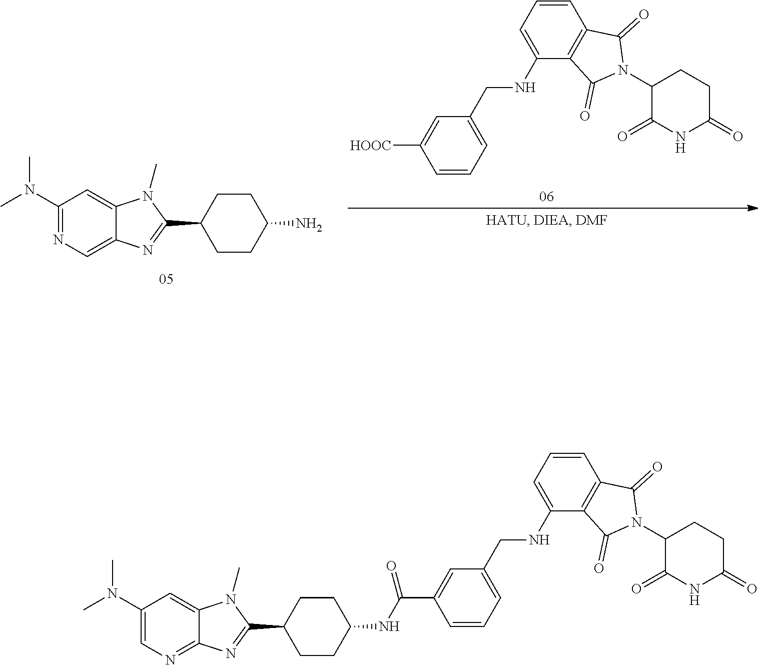 [SMILES: CN(C)c1cc2c(cn1)nc([C@H]1CC[C@H](N)CC1)n2C ; O=C1CCC(N2C(=O)c3cccc(NCc4cccc(C(=O)O)c4)c3C2=O)C(=O)N1 ; CN(C)c1cnc2nc([C@H]3CC[C@H](NC(=O)c4cccc(CNc5cccc6c5C(=O)N(C5CCC(=O)NC5=O)C6=O)c4)CC3)n(C)c2c1]

To a 50 mL single-neck flask were added 05 (40 mg, 0.146 mmol), 06 (53.660 mg, 0.1314 mmol), 2-(7-azabenzotriazol)-N,N,N,N-tetramethyluronium hexafluorophosphate (83.27 mg, 0.219 mmol), N,N-diisopropylethylamine (56.61 mg, 0.438 mmol) and N,N-dimethylformamide (1 mL), and the reaction mixture was reacted at room temperature for 12 h and purified by preparative high performance liquid chromatography to give a yellow solid (7.85 mg, 8.1% yield). LC-MS: 664.4 [M+H]⁺.

¹H NMR (400 MHz, DMSO-d₆) δ 11.11 (s, 1H), 8.60 (t, J=6.1 Hz, 2H), 8.52 (s, 1H), 8.04 (s, 1H), 7.57 (d, J=4.5 Hz, 1H), 7.52-7.44 (m, 2H), 7.02 (dd, J=28.0, 13.0 Hz, 2H), 6.87 (d, J=8.3 Hz, 1H), 5.09 (dd, J=13.0, 5.1 Hz, 1H), 4.71 (d, J=6.2 Hz, 2H), 3.86 (s, 1H), 3.78 (s, 3H), 3.16 (s, 6H), 2.98-2.87 (m, 2H), 2.60 (d, J=21.9 Hz, 3H), 2.07 (d, J=10.7 Hz, 2H), 1.97 (s, 2H), 1.67 (dd, J=21.5, 11.7 Hz, 4H).

### Example 107

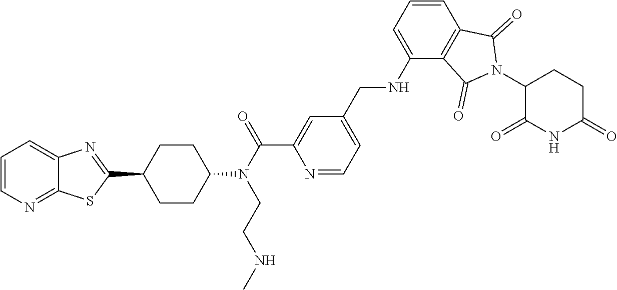 [SMILES: CNCCN(C(=O)c1cc(CNc2cccc3c2C(=O)N(C2CCC(=O)NC2=O)C3=O)ccn1)[C@H]1CC[C@H](c2nc3cccnc3s2)CC1]

### Preparation Steps

### Step 1

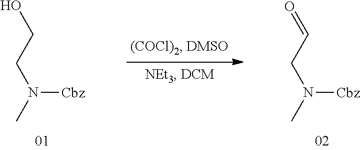

Oxalyl chloride (3.94 g, 31.1 mmol) was dissolved in anhydrous dichloromethane (10 mL) at room temperature, and a mixed solution of dimethyl sulfoxide (4.85 g, 62.1 mmol) and anhydrous dichloromethane (10 mL) was added at −78° C. After the mixture was stirred for 10 min, a mixed solution of 01 (5.00 g, 23.9 mmol) and anhydrous dichloromethane (10 mL) was added dropwise. After the mixture was stirred at that temperature for 1 h, triethylamine (12.1 g, 119.5 mmol) was added. After being stirred for 10 min, the above mixture was heated to room temperature and stirred for 2 h. Water (150 mL) was added, followed by extraction with dichloromethane (30 mL×3). The organic phase was washed with saturated brine (50 mL), dried over anhydrous sodium sulfate, and filtered, and the filtrate was concentrated to dryness by rotary evaporation to give the target product as a yellow oil (4.51 g, 91.0% yield).

### Step 2

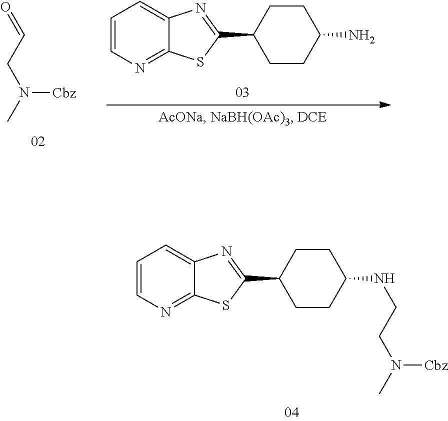 [SMILES: CN(CC=O)C(=O)OCc1ccccc1 ; N[C@H]1CC[C@H](c2nc3cccnc3s2)CC1 ; CN(CCN[C@H]1CC[C@H](c2nc3cccnc3s2)CC1)C(=O)OCc1ccccc1]

02 (50.0 mg, 0.241 mmol), 03 (65.0 mg, 0.241 mmol) and sodium acetate (65.6 mg, 0.482 mmol) were dissolved in dichloroethane (3 mL) at room temperature. After the mixture was stirred for 30 min, sodium triacetoxyborohydride (153 mg, 0.723 mmol) was added. The above mixture was stirred at room temperature for 16 h. The mixture was purified through a silica gel column (dichloromethane:methanol=10:1) to separate the target product as a white solid (45 mg, 43.9% yield). LC-MS: 425.4 [M+H]⁺.

### Step 3

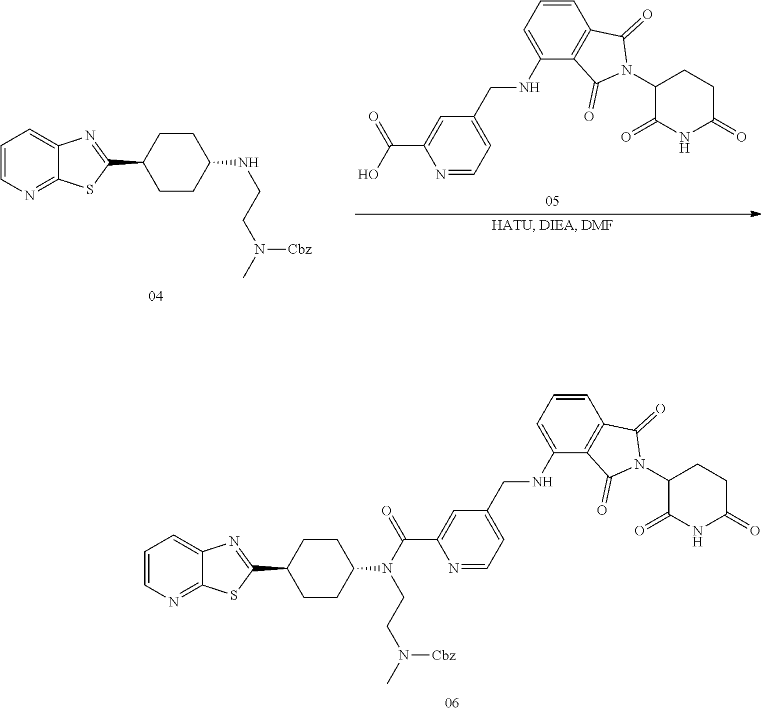 [SMILES: CN(CCN[C@H]1CC[C@H](c2nc3cccnc3s2)CC1)C(=O)OCc1ccccc1 ; O=C1CCC(N2C(=O)c3cccc(NCc4ccnc(C(=O)O)c4)c3C2=O)C(=O)N1 ; CN(CCN(C(=O)c1cc(CNc2cccc3c2C(=O)N(C2CCC(=O)NC2=O)C3=O)ccn1)[C@H]1CC[C@H](c2nc3cccnc3s2)CC1)C(=O)OCc1ccccc1]

04 (45.0 mg, 0.106 mmol), 05 (43.3 mg, 0.106 mmol) and N,N-diisopropylethylamine (41.1 mg, 0.318 mmol) were mixed in anhydrous N,N-dimethylformamide (2 mL) at room temperature, and (7-azabenzotriazole)-tetramethyluronium hexafluorophosphate (60.5 mg, 0.159 mmol) was added with stirring at room temperature. The reaction mixture was stirred at room temperature for 16 h. The mixture was passed through a reversed-phase column (C18, 10-100% acetonitrile/water as mobile phase, 0.1% formic acid) to separate the target product as a green solid (17 mg, 19.7% yield). LC-MS: 815.5 [M+H]⁺.

### Step 4

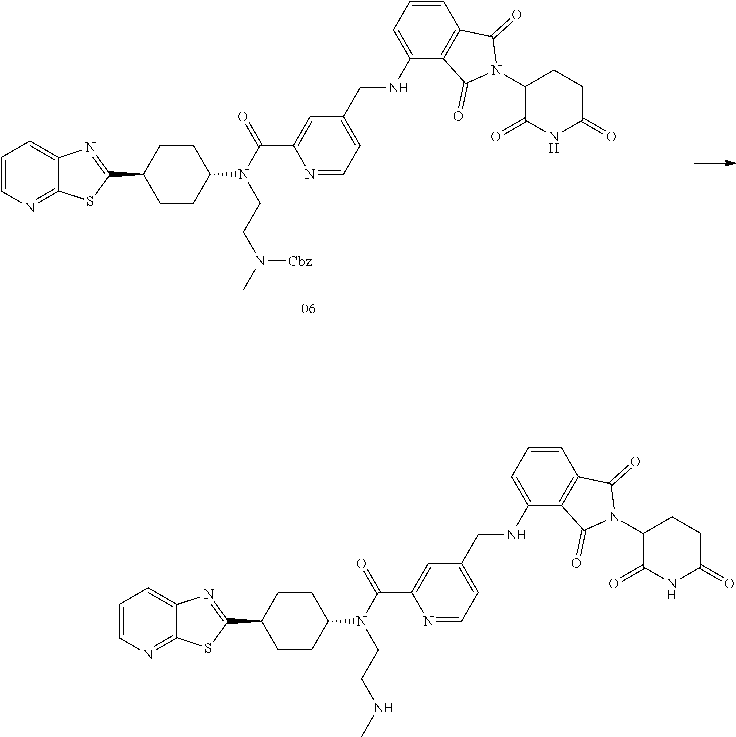 [SMILES: CN(CCN(C(=O)c1cc(CNc2cccc3c2C(=O)N(C2CCC(=O)NC2=O)C3=O)ccn1)[C@H]1CC[C@H](c2nc3cccnc3s2)CC1)C(=O)OCc1ccccc1 ; CNCCN(C(=O)c1cc(CNc2cccc3c2C(=O)N(C2CCC(=O)NC2=O)C3=O)ccn1)[C@H]1CC[C@H](c2nc3cccnc3s2)CC1]

06 (13.0 mg, 0.016 mmol) was dissolved in hydrobromic acid (1 mL) at room temperature. The above mixture was stirred at room temperature for 30 min and concentrated to dryness by rotary evaporation, and the residue was purified by preparative chromatography (C18, 5-100% acetonitrile/water as mobile phase, 0.1% formic acid) to separate the target product as a green solid (1.07 mg, 9.85% yield). LC-MS: 681.3 [M+H]⁺, 1H NMR (400 MHz, DMSO-d₆) δ 11.10 (s, 1H), 8.59-8.48 (m, 2H), 8.36-8.28 (m, 1H), 8.26 (s, 1H), 7.59-7.52 (m, 1H), 7.52-7.40 (m, 4H), 7.07-6.97 (m, 1H), 6.93-6.87 (m, 1H), 5.12-5.05 (m, 1H), 4.67 (d, J=5.7 Hz, 2H), 3.51-3.47 (m, 3H), 3.10-3.04 (m, 1H), 2.94-2.77 (m, 3H), 2.63-2.55 (m, 2H), 2.42 (s, 2H), 2.33-2.26 (m, 1H), 2.15-1.93 (m, 4H), 1.90-1.73 (m, 4H), 1.34-1.23 (m, 1H).

### Example 108

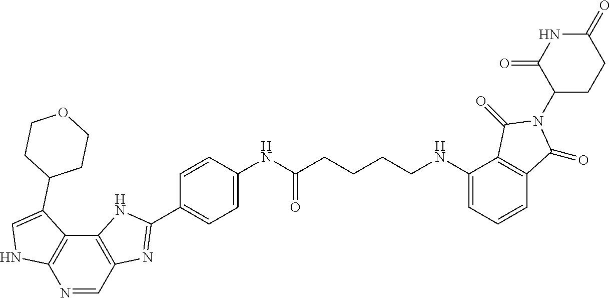 [SMILES: O=C1CCC(N2C(=O)c3cccc(NCCCCC(=O)Nc4ccc(-c5nc6cnc7[nH]cc(C8CCOCC8)c7c6[nH]5)cc4)c3C2=O)C(=O)N1]

### Preparation Steps

### Step 1

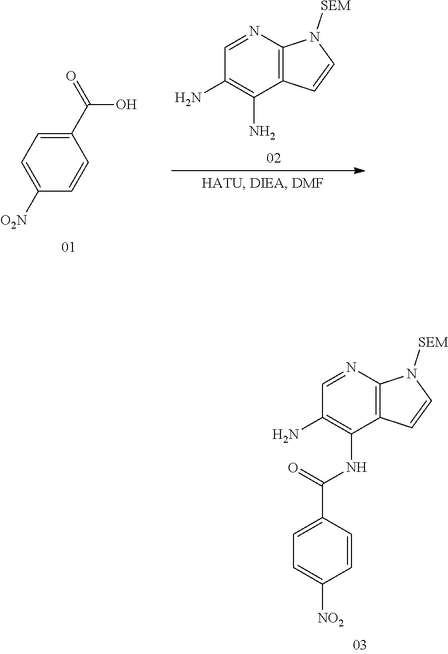 [SMILES: O=C(O)c1ccc([N+](=O)[O-])cc1 ; C[Si](C)(C)CCOCn1ccc2c(N)c(N)cnc21 ; C[Si](C)(C)CCOCn1ccc2c(NC(=O)c3ccc([N+](=O)[O-])cc3)c(N)cnc21]

To a 100 mL single-neck flask were added 01 (300 mg, 1.80 mmol), 02 (500 mg, 1.80 mmol), HATU (1.02 g, 2.69 mmol), N,N-diisopropylethylamine (696 mg, 5.39 mmol) and N,N-dimethylformamide (8 mL), and the mixture was reacted at room temperature for 16 h, quenched with water (20 mL), and extracted with ethyl acetate (40 mL). The organic phase was washed with saturated brine (20 mL×3) and concentrated to dryness by rotary evaporation, and the residue was purified by column chromatography (petroleum ether:ethyl acetate=1:1) to give the target product as a brown solid (700 mg, 91.2% yield). LC-MS: 428.1 [M+H]⁺.

### Step 2

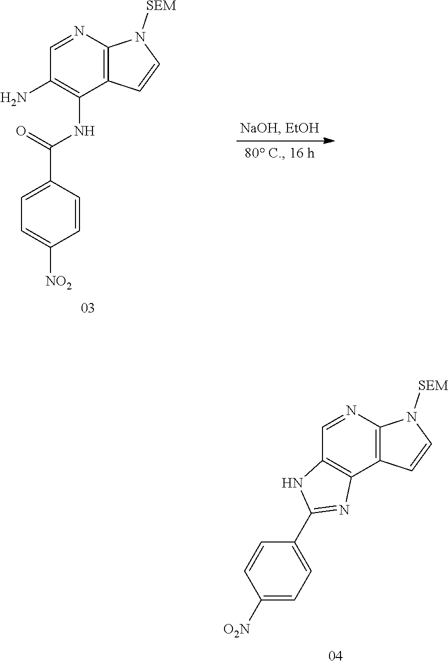 [SMILES: C[Si](C)(C)CCOCn1ccc2c(NC(=O)c3ccc([N+](=O)[O-])cc3)c(N)cnc21 ; C[Si](C)(C)CCOCn1ccc2c3nc(-c4ccc([N+](=O)[O-])cc4)[nH]c3cnc21]

In a 100 mL single-neck round-bottom flask, 03 (700 mg, 1.64 mmol) and sodium hydroxide (196 mg, 4.91 mmol) were dissolved in ethanol (10 mL), and the mixture was reacted at 80° C. for 16 h and concentrated to dryness by rotary evaporation. The residue was purified by column chromatography (dichloromethane:methanol=20:1) to give the target product as a brown solid (520 mg, 77.6% yield). LC-MS: 410.2 [M+H]⁺.

### Step 3

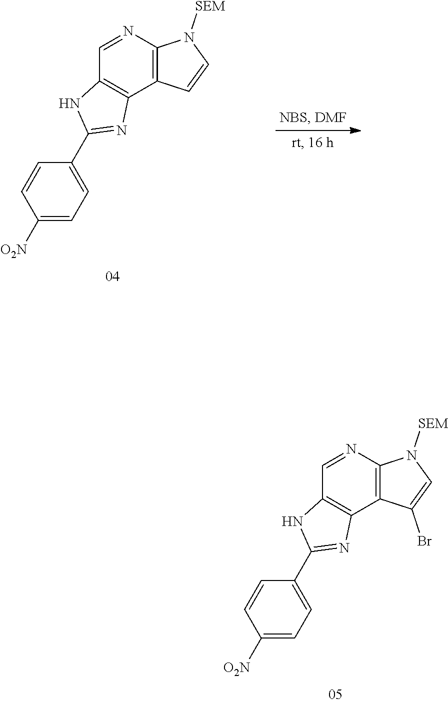 [SMILES: C[Si](C)(C)CCOCn1ccc2c3nc(-c4ccc([N+](=O)[O-])cc4)[nH]c3cnc21 ; C[Si](C)(C)CCOCn1cc(Br)c2c3nc(-c4ccc([N+](=O)[O-])cc4)[nH]c3cnc21]

In a 100 mL single-neck flask, 04 (520 mg, 1.27 mmol) was dissolved in N,N-dimethylformamide (5 mL), and N-bromosuccinimide (203 mg, 1.14 mmol) was added. The reaction mixture was stirred at room temperature for 16 h and purified by column chromatography (dichloromethane:methanol=20:1) to give the target product as a brown solid (530 mg, 85.5% yield). LC-MS: 487.9/489.9 [M+H]⁺.

### Step 4

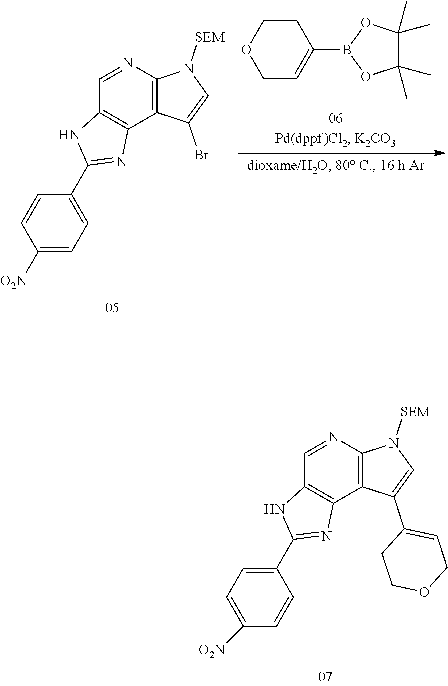 [SMILES: C[Si](C)(C)CCOCn1cc(Br)c2c3nc(-c4ccc([N+](=O)[O-])cc4)[nH]c3cnc21 ; CC1(C)OB(C2=CCOCC2)OC1(C)C ; C[Si](C)(C)CCOCn1cc(C2=CCOCC2)c2c3nc(-c4ccc([N+](=O)[O-])cc4)[nH]c3cnc21]

In a 100 mL single-neck flask, 05 (530 mg, 1.09 mmol) was dissolved in 1,4-dioxane (10 mL) and water (2 mL), and 06 (342 mg, 1.63 mmol), potassium carbonate (450 mg, 3.26 mmol) and Pd(dppf)Cl₂ (40 mg, 0.0543 mmol) were added. The mixture was reacted in an argon atmosphere at 80° C. for 16 h and purified by reversed-phase chromatography (acetonitrile:water=77%) to give the target product as a brown solid (210 mg, 39.4% yield). LC-MS: 492.2 [M+H]⁺.

### Step 5

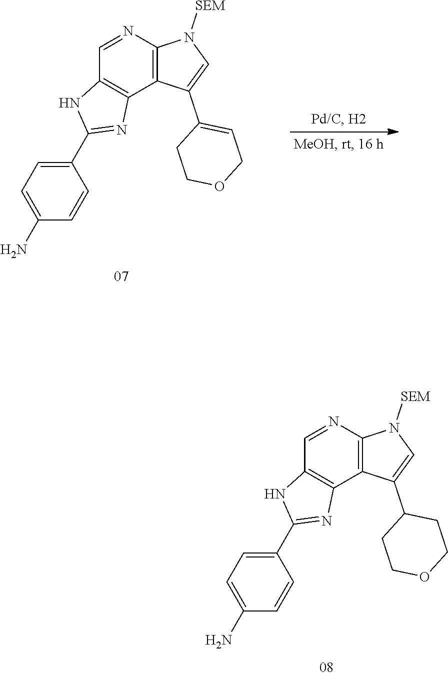 [SMILES: C[Si](C)(C)CCOCn1cc(C2=CCOCC2)c2c3nc(-c4ccc(N)cc4)[nH]c3cnc21 ; C[Si](C)(C)CCOCn1cc(C2CCOCC2)c2c3nc(-c4ccc(N)cc4)[nH]c3cnc21]

In a 100 mL single-neck flask, 07 (210 mg, 0.427 mmol) was dissolved in methanol (10 mL), and palladium on carbon (20 mg) was added. The mixture was stirred in a hydrogen atmosphere for 16 h and filtered, and the filtrate was concentrated to dryness by rotary evaporation to give the target product as a brown solid (163 mg, 82.3% yield). LC-MS: 464.2 [M+H]⁺.

### Step 6

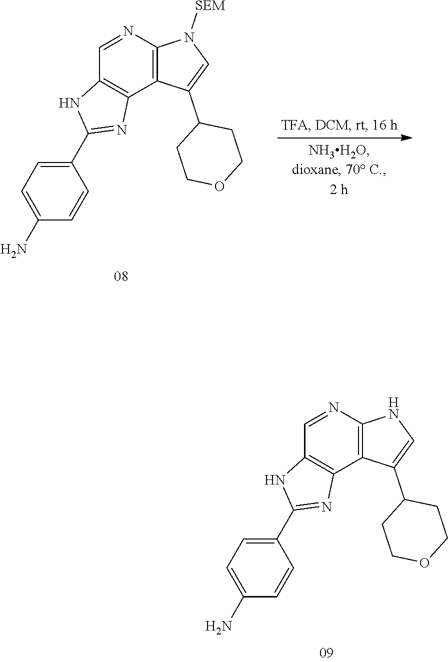 [SMILES: C[Si](C)(C)CCOCn1cc(C2CCOCC2)c2c3nc(-c4ccc(N)cc4)[nH]c3cnc21 ; Nc1ccc(-c2nc3c(cnc4[nH]cc(C5CCOCC5)c43)[nH]2)cc1]

In a 50 mL single-neck flask, 08 (163 mg, 0.352 mmol) was dissolved in dichloromethane (5 mL), and trifluoroacetic acid (0.5 mL) was added. The reaction mixture was stirred at room temperature for 16 h. After the reaction mixture was concentrated to dryness by rotary evaporation, 1,4-dioxane (5 mL) and ammonia solution (1 mL) were added. The reaction mixture was stirred at 70° C. for 1 h and concentrated to dryness by rotary evaporation, and the residue was purified by preparative reversed-phase liquid chromatography to give the target product as a white solid (20 mg, 17.1% yield). LC-MS: 334.1 [M+H]⁺.

### Step 7

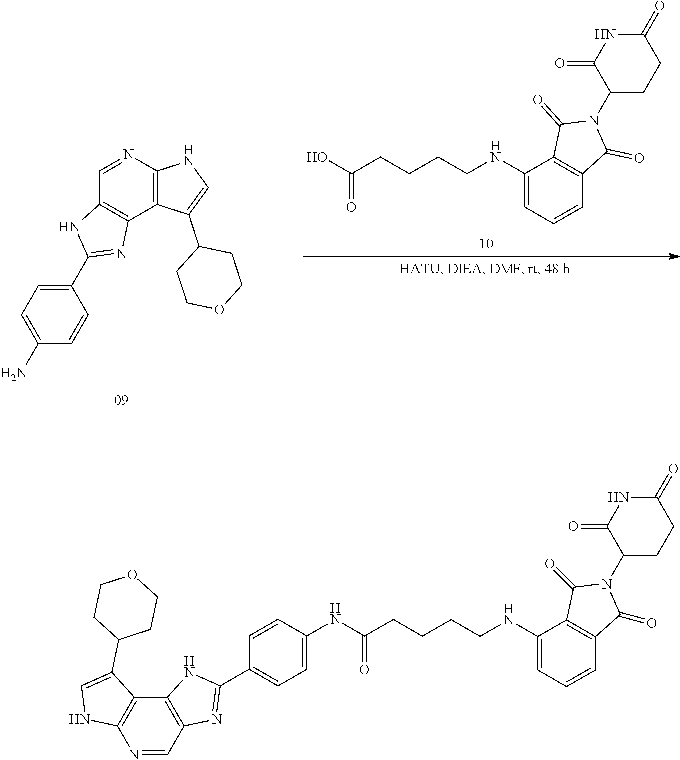 [SMILES: Nc1ccc(-c2nc3c(cnc4[nH]cc(C5CCOCC5)c43)[nH]2)cc1 ; O=C(O)CCCCNc1cccc2c1C(=O)N(C1CCC(=O)NC1=O)C2=O ; O=C1CCC(N2C(=O)c3cccc(NCCCCC(=O)Nc4ccc(-c5nc6cnc7[nH]cc(C8CCOCC8)c7c6[nH]5)cc4)c3C2=O)C(=O)N1]

To a 25 mL single-neck flask were added 09 (20 mg, 0.0600 mmol), 10 (22.4 mg, 0.0600 mmol), HATU (34.2 mg, 0.0900 mmol), N,N-diisopropylethylamine (23.3 mg, 0.180 mmol) and N,N-dimethylformamide (8 mL), and the mixture was reacted at room temperature for 16 h and purified by preparative reversed-phase liquid chromatography to give the target product as a brown solid (2.11 mg, 5.1% yield). LC-MS: 689.2 [M+H]⁺.

¹H NMR (400 MHz, DMSO-d₆) δ 13.02 (s, 0.65H), 12.51 (s, 0.24H), 11.46 (s, 0.23H), 11.29 (s, 0.69H), 11.08 (s, 1H), 10.16 (s, 1H), 8.55 (s, 0.23H), 8.46 (s, 0.70H), 8.15 (d, J=8.4 Hz, 2H), 7.79 (d, J=8.4 Hz, 2H), 7.58 (t, J=7.7 Hz, 1H), 7.13 (d, J=8.8 Hz, 2H), 7.02 (d, J=7.0 Hz, 1H), 6.61 (s, 1H), 5.05 (dd, J=13.0, 5.1 Hz, 1H), 4.00 (s, 2H), 3.59 (s, 2H), 3.36 (s, 2H), 2.88 (t, J=12.8 Hz, 1H), 2.58 (d, J=16.7 Hz, 2H), 2.41 (d, J=6.9 Hz, 2H), 2.23-1.84 (m, 5H), 1.80-1.52 (m, 5H).

### Example 109

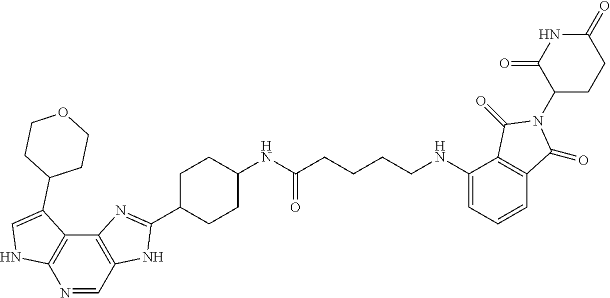 [SMILES: O=C1CCC(N2C(=O)c3cccc(NCCCCC(=O)NC4CCC(c5nc6c(cnc7[nH]cc(C8CCOCC8)c76)[nH]5)CC4)c3C2=O)C(=O)N1]

### Preparation Steps

### Step 1

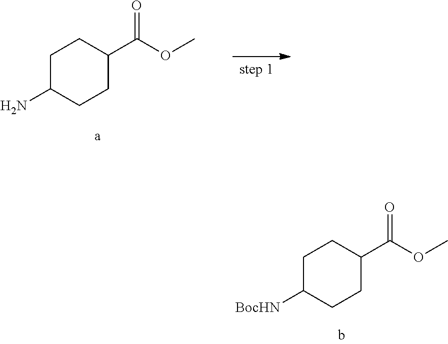 [SMILES: COC(=O)C1CCC(N)CC1 ; "step 1" ; COC(=O)C1CCC(NC(=O)OC(C)(C)C)CC1]

In a 3 L single-neck flask, a (100 g, 0.516 mol) was dissolved in DMF (1 L), and (Boc)₂O (562.8 g, 2.58 mol), TEA (526.7 g, 5.16 mol) and DMAP (31.5 g, 0.258 mol) were added. The mixture was reacted at 70° C. for 18 h and filtered under vacuum, and the filtrate was concentrated, purified by column chromatography (petroleum ether:ethyl acetate=60:1), and concentrated under reduced pressure to give the target product as a yellow oil (130 g, 97.9% yield). LC-MS: 258 [M+H]⁺.

### Step 2

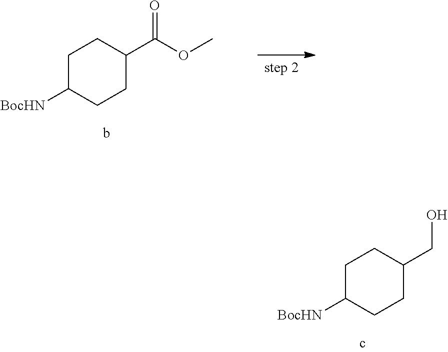 [SMILES: COC(=O)C1CCC(NC(=O)OC(C)(C)C)CC1 ; "step 2" ; CC(C)(C)OC(=O)NC1CCC(CO)CC1]

In a 3 L three-necked flask, LiAlH₄ (57.6 g, 1.515 mmol) was dissolved in THF (1.2 L) and cooled to −5° C. b (130 g, 0.505 mmol) was dissolved in THF (800 mL), and the resulting solution was added dropwise slowly to the system. The reaction system was reacted at 0° C. for 1 h, quenched with ice water, washed with 15% aqueous NaOH solution followed by ice water. The mixture was filtered under vacuum, and the filtrate was concentrated and extracted with ethyl acetate (500 mL×3). The organic phase was washed with saturated brine (200 mL×3), dried over anhydrous sodium sulfate, and concentrated under reduced pressure to give the target product as a colorless oil (98 g). LC-MS: 230 [M+H]⁺.

### Step 3

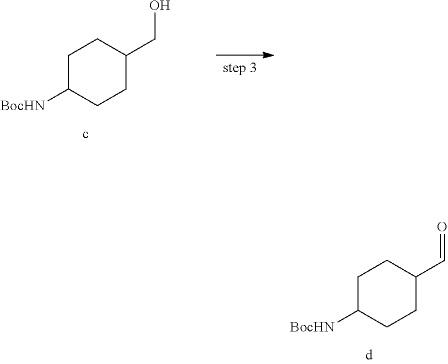 [SMILES: CC(C)(C)OC(=O)NC1CCC(CO)CC1 ; "step 3" ; CC(C)(C)OC(=O)NC1CCC(C=O)CC1]

In a 3 L three-necked flask, c (98 g, 0.427 mol) was dissolved in DCM (1.5 L), and the solution was cooled to 0° C. in an ice bath. Dess-Martin periodinane (181.3 g, 0.427 mol) was added, and the mixture was reacted at room temperature for 1 h and filtered under vacuum. The filtrate was quenched with saturated aqueous NaHCO₃ solution and extracted with dichloromethane (200 mL×3), and the organic phase was washed with saturated brine (200 mL×2), dried over anhydrous sodium sulfate, purified by column chromatography, and concentrated under reduced pressure to give the target product as a yellow oil (62 g). LC-MS: 228 [M+H]⁺.

### Step 4

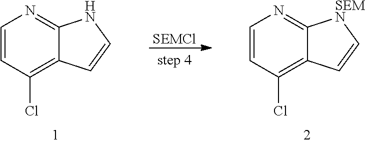 [SMILES: Clc1ccnc2[nH]ccc12 ; "SEMCl" ; C[Si](C)(C)CCOCn1ccc2c(Cl)ccnc21]

In a 2 L three-necked flask, 1 (100 g, 0.655 mol) was dissolved in DMF (1 L), and the solution was cooled to 0° C. in an ice bath. NaH (39.4 g, 0.98 mol) was added, and the mixture was reacted at room temperature for 1 h and cooled to 0° C. in an ice bath. SEMCl (131.1 g, 0.786 mol) was added. The reaction mixture was quenched with water and extracted with ethyl acetate (200 mL×3), and the organic phase was washed with saturated brine (200 mL×2), dried over anhydrous sodium sulfate, purified by column chromatography, and concentrated under reduced pressure to give the target product as a yellow oil (170 g). LC-MS: 283 [M+H]⁺.

### Step 5

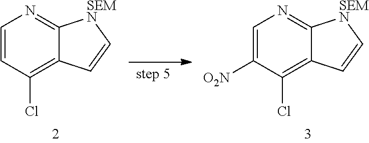 [SMILES: C[Si](C)(C)CCOCn1ccc2c(Cl)ccnc21 ; C[Si](C)(C)CCOCn1ccc2c(Cl)c([N+](=O)[O-])cnc21]

In a 3 L three-necked flask, 2 (170 g, 0.6 mol) was dissolved in DMF (1.5 L), cooled to 0° C. in an ice bath, and tetrabutylammonium nitrate (274.5 g, 0.9 mol) and trifluoroacetic anhydride (195.7 g, 0.93 mol) were added. The mixture was reacted in a nitrogen atmosphere at room temperature for 20 h, quenched with saturated aqueous NaHCO₃ solution, and extracted with dichloromethane (200 mL×3), and the organic phase was washed with saturated brine (200 mL×2), dried over anhydrous sodium sulfate, and concentrated to give the target product as a yellow oil (130 g). LC-MS: 328 [M+H]⁺.

### Step 6

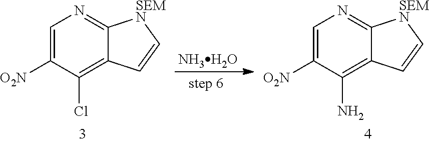

In a 2 L single-neck flask, 3 (130 g, 0.397 mol) was dissolved in 1,4-dioxane (1100 mL), and ammonia solution (1100 mL) was added. The mixture was reacted at 80° C. overnight, quenched with water, concentrated, and extracted with dichloromethane (200 mL×3), and the organic phase was washed with saturated brine (200 mL×2), dried over anhydrous sodium sulfate, and concentrated under reduced pressure to give the target product as a yellow oil (70 g). LC-MS: 309 [M+H]⁺.

### Step 7

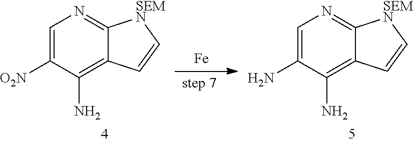

In a 2 L single-neck flask, 4 (70 g, 0.227 mol) was dissolved in ethanol (1.4 L) and water (280 mL), and iron powder (63.6 g, 1.2 mol) and ammonium chloride (60.22 g, 1.2 mol) were added. The mixture was reacted at 80° C. for 2 h, cooled to room temperature, and filtered, and the filtrate was extracted with dichloromethane (200 mL×3). The organic phase was dried over anhydrous sodium sulfate, purified by column chromatography, and concentrated under reduced pressure to give the target product as a yellow oil (35 g). LC-MS: 279 [M+H]⁺.

### Step 8

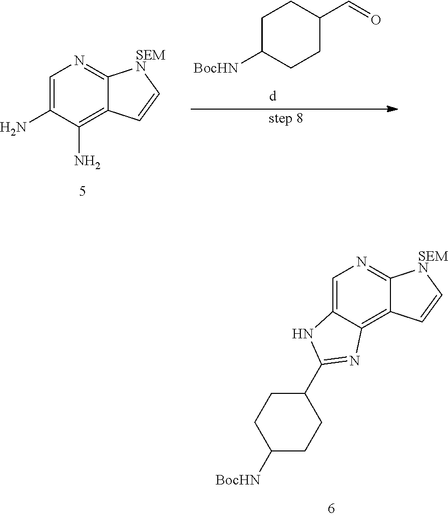 [SMILES: C[Si](C)(C)CCOCn1ccc2c(N)c(N)cnc21 ; CC(C)(C)OC(=O)NC1CCC(C=O)CC1 ; "step 8" ; CC(C)(C)OC(=O)NC1CCC(c2nc3c(cnc4c3ccn4COCC[Si](C)(C)C)[nH]2)CC1]

To a 3 L single-neck flask were added 5 (35 g, 0.126 mol), d (42.86 g, 0.188 mmol), AcOH (700 mL) and methanol (1.4 L), and the mixture was reacted in an oxygen atmosphere at room temperature for 16 h, concentrated, quenched with ice water, and extracted with dichloromethane (200 mL×4). The organic phase was washed with saturated brine (2000 mL×2), dried over anhydrous sodium sulfate, purified by column chromatography, and concentrated under reduced pressure to give the target product as a yellow oil (31 g). LC-MS: 486 [M+H]⁺.

### Step 9

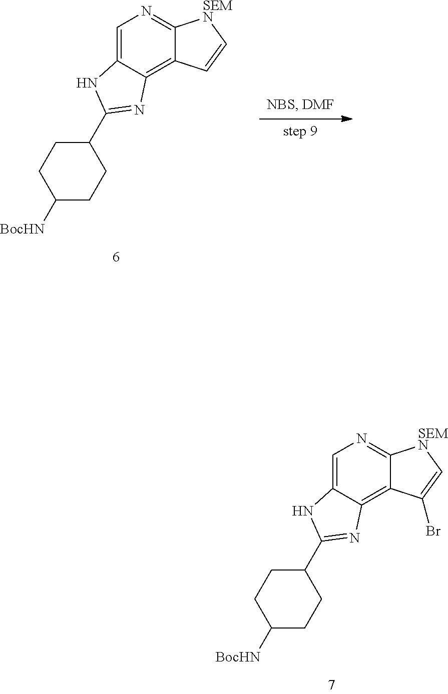

To a 1 L single-neck flask were added 6 (31 g, 63.8 mmol), NBS (13.6 g, 76.6 mmol) and DMF (350 mL), and the mixture was reacted at room temperature for 16 h, quenched with ice water, and extracted with ethyl acetate (200 mL×3), The organic phase was washed with saturated brine (100 mL×3), dried over anhydrous sodium sulfate, purified by column chromatography, and concentrated under reduced pressure to give the target product as a yellow oil (16 g). LC-MS: 564 [M+H]⁺.

### Step 10

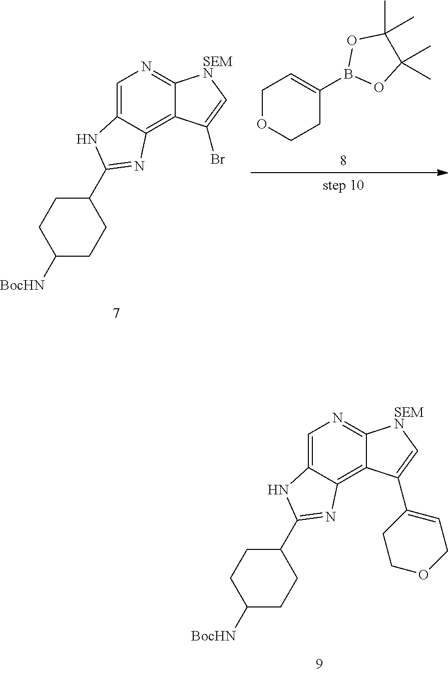

To a 500 mL single-neck flask were added 7 (16 g, 28.3 mmol), 8 (10.12 g, 48.2 mmol), Pd₂(dppf)Cl₂·CH₂Cl₂ (2.34 g, 2.83 mmol), K₂CO₃ (11.7 g, 85.0 mmol), dioxane (170 mL) and H₂O (34 mL), and the mixture was reacted in a nitrogen atmosphere at 80° C. for 16 h, and concentrated to dryness by rotary evaporation. The residue was purified by column chromatography (dichloromethane:methanol=20:1), followed by concentration under reduced pressure to give the target product as a yellow oil (12 g). LC-MS: 568 [M+H]⁺.

### Step 11

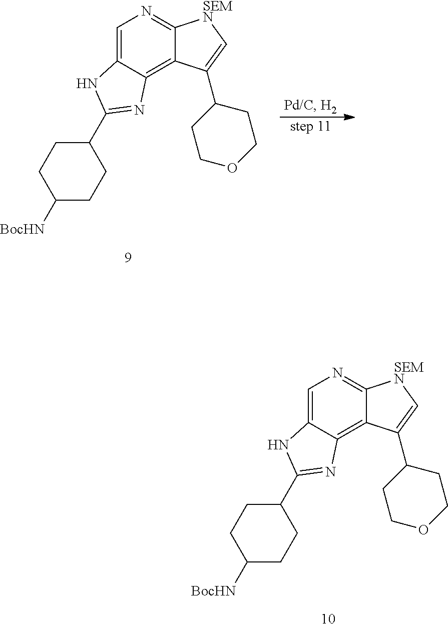 [SMILES: CC(C)(C)OC(=O)NC1CCC(c2nc3c(cnc4c3c(C3CCOCC3)cn4COCC[Si](C)(C)C)[nH]2)CC1 ; "Pd/C, H2" ; "step 11" ; CC(C)(C)OC(=O)NC1CCC(c2nc3c(cnc4c3c(C3CCOCC3)cn4COCC[Si](C)(C)C)[nH]2)CC1 ; "10"]

To a 1 L hydrogenation reactor were added 9 (12 mg, 21.12 mmol), Pd(OH)₂ (3.8 g) and MeOH (300 mL), and the mixture was reacted in a hydrogen atmosphere at room temperature for 16 h and filtered under vacuum. The filtrate was concentrated to give the target product as a yellow oil (11 g). LC-MS: 570 [M+H]⁺.

### Step 12

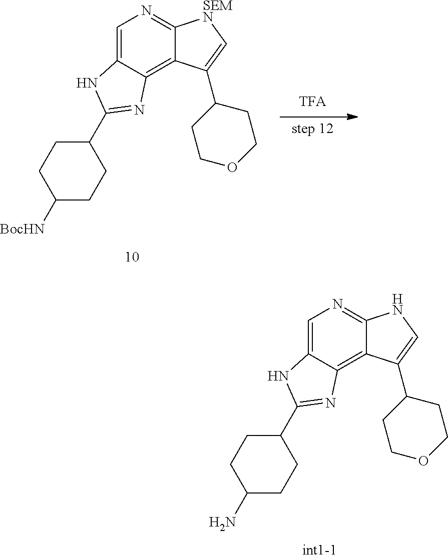 [SMILES: CC(C)(C)OC(=O)NC1CCC(c2nc3c(cnc4c3c(C3CCOCC3)cn4COCC[Si](C)(C)C)[nH]2)CC1 ; "step 12" ; NC1CCC(c2nc3c(cnc4[nH]cc(C5CCOCC5)c43)[nH]2)CC1 ; "TFA"]

To a 1 L single-neck flask were added 10 (11 g, 19.3 mmol), TFA (100 mL) and DCM (300 mL), and the mixture was reacted at room temperature for 16 h and concentrated to give a crude product as a yellow oil (300 mg), followed by addition of NH₃H₂O (40 mL) and dioxane (200 mL). The mixture was reacted at 70° C. for 16 h and concentrated to dryness by rotary evaporation, and the residue was purified by Prep-HPLC (ACN-H₂O, TFA) to give the target product as a yellow oil (3.5 g). LC-MS: 340 [M+H]⁺.

### Step 13

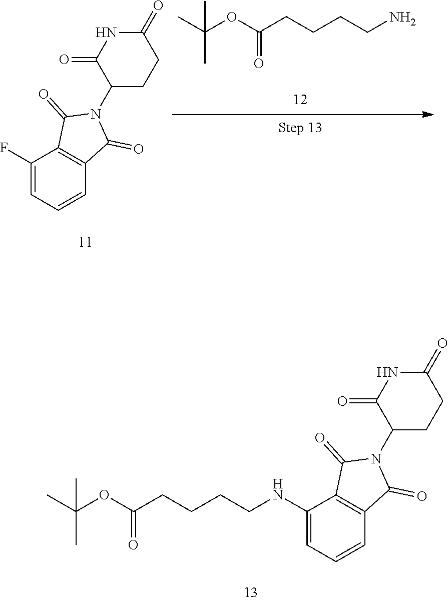

11 (200 mg, 0.724 mmol), 12 (150.5 mg, 0.869 mmol) and N-methylpyrrolidone (2 mL) were added to a 100 mL single-neck flask and mixed well by stirring, and DIEA (468 mg, 3.62 mmol) was added. The mixture was reacted at 110° C. for 18 h and concentrated to dryness by rotary evaporation, and the residue was purified by column chromatography to give the target product as a yellowish-green solid (150 mg, 48.24%). LC-MS: 430 [M+H]⁺.

### Step 14

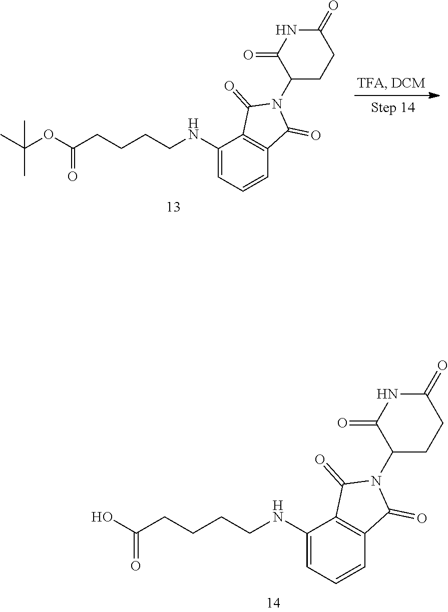 [SMILES: CC(C)(C)OC(=O)CCCCNc1cccc2c1C(=O)N(C1CCC(=O)NC1=O)C2=O ; O=C(O)CCCCNc1cccc2c1C(=O)N(C1CCC(=O)NC1=O)C2=O]

In a 100 mL single-neck flask, 13 (150 mg, 0.35 mmol) was dissolved in DCM (2 mL), and TFA (0.4 mL) was added. The mixture was reacted at room temperature for 16 h and concentrated to dryness by rotary evaporation, and the residue was purified by column chromatography to give the target product as a yellowish-green solid (110 mg, 84.35%). LC-MS: 374 [M+H]⁺.

### Step 15

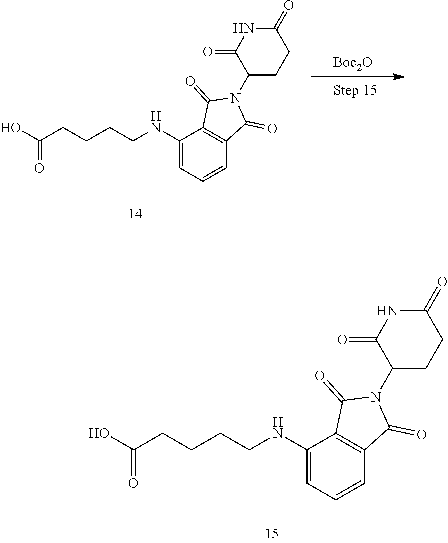 [SMILES: "Boc2O" ; O=C(O)CCCCNc1cccc2c1C(=O)N(C1CCC(=O)NC1=O)C2=O ; O=C(O)CCCCNc1cccc2c1C(=O)N(C1CCC(=O)NC1=O)C2=O]

In a 100 mL single-neck flask, 14 (110 mg, 0.295 mmol) was dissolved in THF (2 mL), and Boc₂O (64.3 mg, 0.295 mmol) and DMAP (3.6 mg, 0.0295 mmol) were added. The mixture was reacted at room temperature for 16 h and concentrated to dryness by rotary evaporation, and the residue was purified by column chromatography to give the target product as a yellowish-green solid (120 mg, 86.02%). LC-MS: 474 [M+H]⁺.

### Step 16

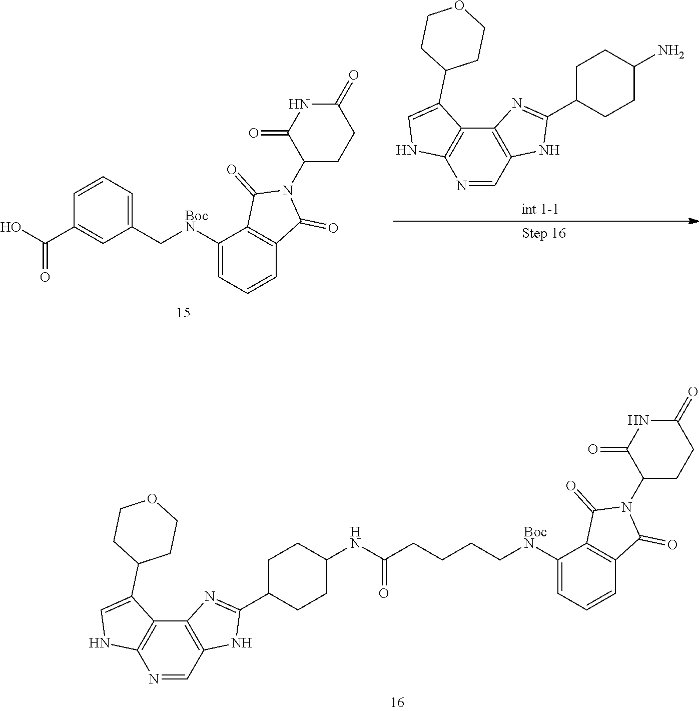 [SMILES: CC(C)(C)OC(=O)N(Cc1cccc(C(=O)O)c1)c1cccc2c1C(=O)N(C1CCC(=O)NC1=O)C2=O ; NC1CCC(c2nc3c(cnc4[nH]cc(C5CCOCC5)c43)[nH]2)CC1 ; CC(C)(C)OC(=O)N(CCCCC(=O)NC1CCC(c2nc3c(cnc4[nH]cc(C5CCOCC5)c43)[nH]2)CC1)c1cccc2c1C(=O)N(C1CCC(=O)NC1=O)C2=O]

In a 100 mL single-neck flask, 15 (70 mg, 0.148 mmol) and int1-1 (60.25 mg, 0.1776 mmol) were dissolved in DMF (2 mL) and mixed well by stirring, and HATU (84.4 mg, 0.222 mmol) and DIEA (57.4 mg, 0.444 mmol) were added. The mixture was reacted at room temperature for 18 h and concentrated to dryness by rotary evaporation, and the residue was purified by column chromatography to give the target product as a yellowish-green solid (45 mg, 38.3%). LC-MS: 795 [M+H]⁺.

### Step 17

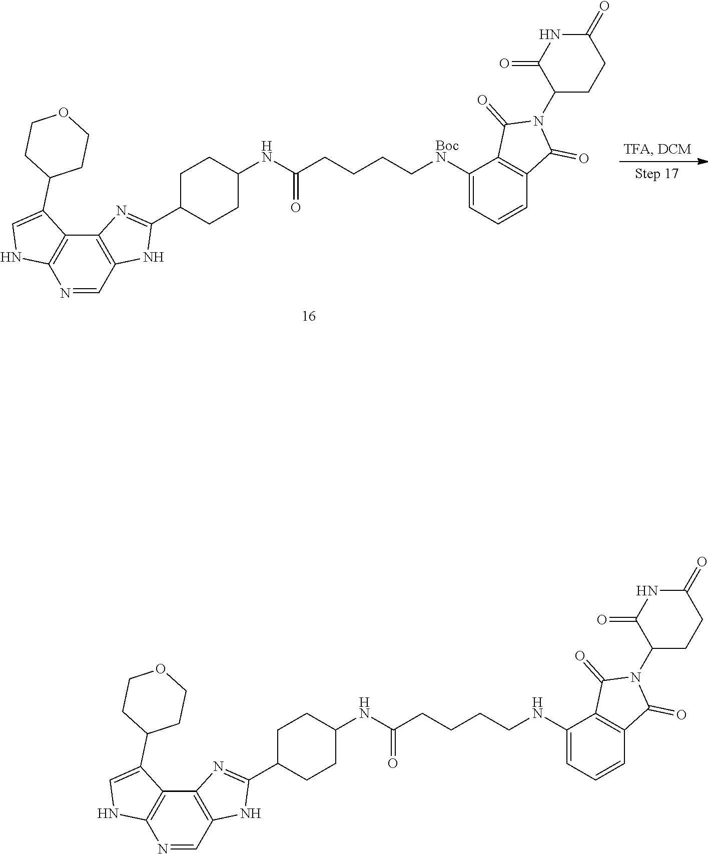 [SMILES: CC(C)(C)OC(=O)N(CCCCC(=O)NC1CCC(c2nc3c(cnc4[nH]cc(C5CCOCC5)c43)[nH]2)CC1)c1cccc2c1C(=O)N(C1CCC(=O)NC1=O)C2=O ; O=C1CCC(N2C(=O)c3cccc(NCCCCC(=O)NC4CCC(c5nc6c(cnc7[nH]cc(C8CCOCC8)c76)[nH]5)CC4)c3C2=O)C(=O)N1]

In a 100 mL single-neck flask, 16 (45 mg, 0.0566 mmol) was dissolved in DCM (2 mL), and TFA (0.4 mL) was added. The mixture was reacted at room temperature for 16 h and concentrated to dryness by rotary evaporation, and the residue was purified by Prep-HPLC, followed by concentration under reduced pressure to give the target product as a yellowish-green solid (5 mg, 12.7%). LC-MS: 695 [M+H]⁺, ¹H NMR (400 MHz, DMSO) δ 12.24 (s, 1H), 11.10 (s, 1H), 8.78 (s, 1H), 7.84 (d, J=7.7 Hz, 1H), 7.59 (dd, J=8.5, 7.2 Hz, 1H), 7.49 (s, 1H), 7.10 (t, J=7.6 Hz, 1H), 7.03 (d, J=7.0 Hz, 1H), 6.59 (s, 1H), 5.06 (dd, J=12.8, 5.4 Hz, 1H), 3.98 (dd, J=10.7, 3.2 Hz, 2H), 3.72-3.57 (m, 3H), 3.37 (dd, J=24.7, 12.7 Hz, 3H), 3.13 (d, J=11.2 Hz, 1H), 2.95-2.81 (m, 1H), 2.64-2.52 (m, 1H), 2.15 (dd, J=26.8, 9.2 Hz, 4H), 2.09-1.82 (m, 6H), 1.75 (dd, J=26.4, 12.6 Hz, 4H), 1.58 (d, J=3.0 Hz, 4H), 1.36 (dd, J=23.6, 11.1 Hz, 2H).

### Example 110

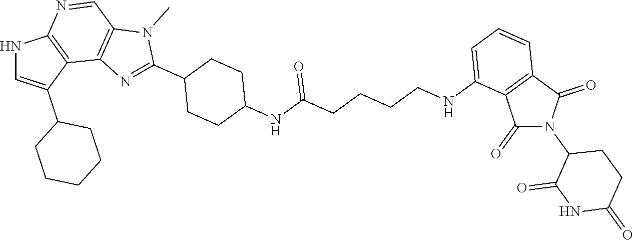 [SMILES: Cn1c(C2CCC(NC(=O)CCCCNc3cccc4c3C(=O)N(C3CCC(=O)NC3=O)C4=O)CC2)nc2c3c(C4CCCCC4)c[nH]c3ncc21]

### Preparation Steps

### Step 1

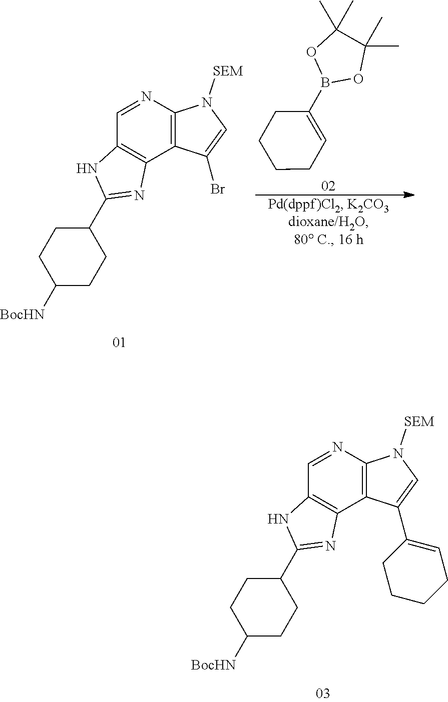

01 (900 mg, 1.59 mmol) was dissolved in 1,4-dioxane:water (12 mL, 5:1), and 02 (332 mg, 1.60 mmol), Pd(dppf)Cl₂ (58 mg, 0.0793 mmol) and potassium carbonate (661 mg, 4.78 mmol) were added successively. The reaction system was heated to 80° C. in an argon atmosphere, stirred for 16 h, and extracted with ethyl acetate (20 mL×3). The organic phases were combined, washed with saturated brine (30 mL), dried over anhydrous sodium sulfate, concentrated, and purified through a reversed-phase column to give a brown solid (450 mg, 49.89% yield). LC-MS: 566 [M+H]⁺.

### Step 2

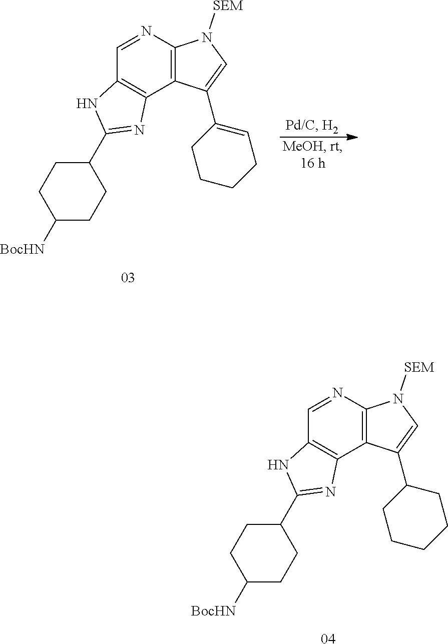 [SMILES: CC(C)(C)OC(=O)NC1CCC(c2nc3c(cnc4c3c(C3=CCCCC3)cn4COCC[Si](C)(C)C)[nH]2)CC1 ; "Pd/C, H2" ; "MeOH, rt,16 h" ; CC(C)(C)OC(=O)NC1CCC(c2nc3c(cnc4c3c(C3CCCCC3)cn4COCC[Si](C)(C)C)[nH]2)CC1 ; "04"]

03 (450 mg, 0.795 mmol) was dissolved in absolute methanol (20 mL) at room temperature, and palladium on carbon (250 mg) was added. The reaction mixture was stirred in a hydrogen atmosphere at room temperature for 16 h. Most starting material was consumed as monitored by LCMS. The reaction mixture was filtered, and the filtrate was concentrated under reduced pressure to give a brown solid (320 mg, 70.86% yield). LC-MS: 568 [M+H]⁺.

### Step 3

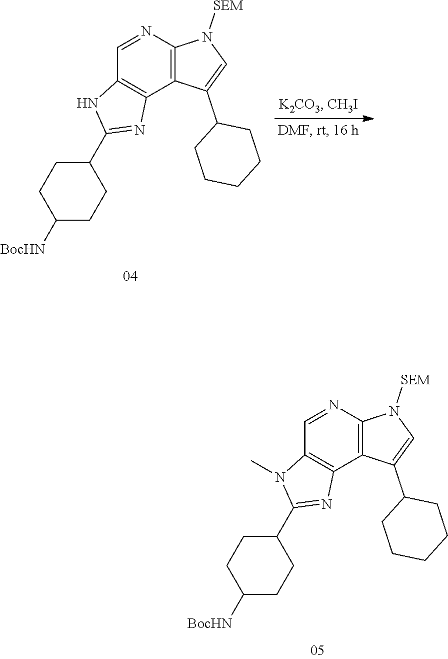 [SMILES: CC(C)(C)OC(=O)NC1CCC(c2nc3c(cnc4c3c(C3CCCCC3)cn4COCC[Si](C)(C)C)[nH]2)CC1 ; Cn1c(C2CCC(NC(=O)OC(C)(C)C)CC2)nc2c3c(C4CCCCC4)cn(COCC[Si](C)(C)C)c3ncc21]

04 (320 mg, 0.564 mmol) was dissolved in N,N-dimethylformamide (5 mL) at room temperature, and potassium carbonate (389 mg, 2.81 mmol) was added, followed by addition of iodomethane (480 mg, 3.38 mmol) after 15 min. The reaction mixture was stirred at room temperature overnight and extracted with ethyl acetate (10 mL×3). The organic phases were combined, washed with saturated brine (10 mL), dried over anhydrous sodium sulfate, then concentrated, and purified by column chromatography (dichloromethane:methanol=20:1) to give a white solid (110 mg, 33.55% yield). LC-MS: 582 [M+H]⁺.

### Step 4

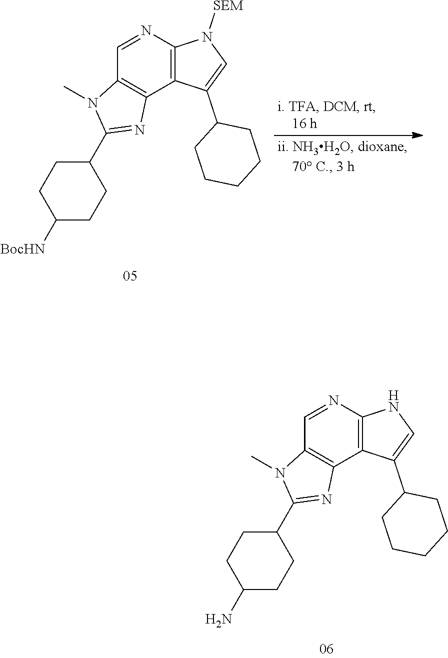 [SMILES: Cn1c(C2CCC(NC(=O)OC(C)(C)C)CC2)nc2c3c(C4CCCCC4)cn(COCC[Si](C)(C)C)c3ncc21 ; Cn1c(C2CCC(N)CC2)nc2c3c(C4CCCCC4)c[nH]c3ncc21]

05 (110 mg, 0.189 mmol) was dissolved in dichloromethane (8 mL) at room temperature, and trifluoroacetic acid (2 mL) was added. The above mixture was stirred at room temperature for 16 h. After the reaction mixture was concentrated to dryness by rotary evaporation, 1,4-dioxane (5 mL) and ammonia solution (1 mL) were added. The reaction mixture was stirred at 70° C. for 3 h and concentrated to dryness by rotary evaporation, and the residue was purified by preparative high performance liquid chromatography to give a brown solid (50 mg, 75.25% yield). LC-MS: 352 [M+H]⁺.

### Step 5

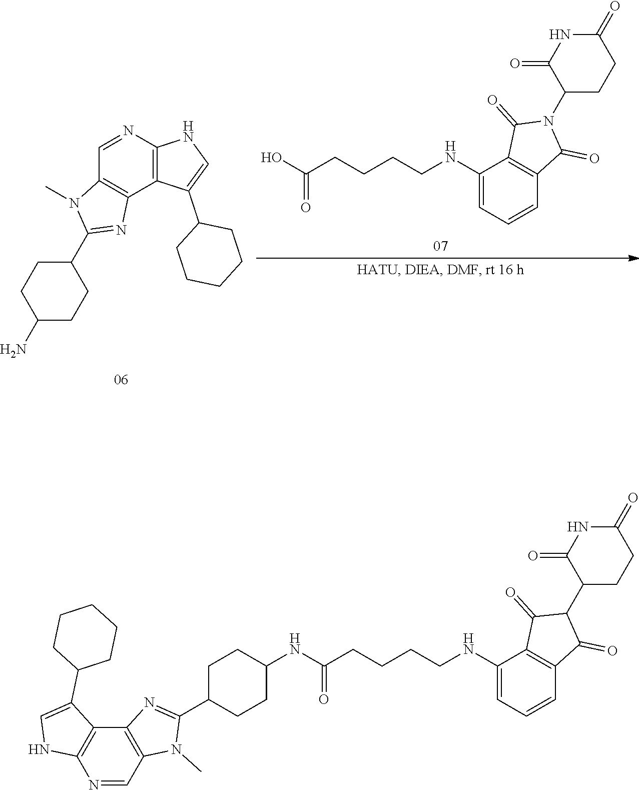 [SMILES: Cn1c(C2CCC(N)CC2)nc2c3c(C4CCCCC4)c[nH]c3ncc21 ; O=C(O)CCCCNc1cccc2c1C(=O)N(C1CCC(=O)NC1=O)C2=O ; Cn1c(C2CCC(NC(=O)CCCCNc3cccc4c3C(=O)C(C3CCC(=O)NC3=O)C4=O)CC2)nc2c3c(C4CCCCC4)c[nH]c3ncc21]

06 (30 mg, 0.0853 mmol) was dissolved in N,N-dimethylformamide (5 mL) at room temperature, and 07 (32 mg, 0.0857 mmol), HATU (49 mg, 0.129 mmol) and N,N-diisopropylethylamine (33 mg, 0.255 mmol) were added successively. The above mixture was stirred at room temperature for 1 h. The reaction mixture was purified twice by preparative high performance liquid chromatography to give a yellowish-green solid (1.95 mg, 3.23% yield).

LC-MS: 707.6 [M+H]^{+ 1}H NMR (400 MHz, DMSO-d₆) δ 12.11 (s, 1H), 11.09 (s, 1H), 8.87 (s, 1H), 7.86 (d, J=7.7 Hz, 1H), 7.59 (t, J=7.7 Hz, 1H), 7.34 (d, J=11.5 Hz, 1H), 7.14-7.01 (m, 2H), 6.58 (s, 1H), 5.05 (dd, J=12.9, 5.2 Hz, 1H), 4.00 (s, 3H), 3.64 (s, 2H), 2.87 (d, J=15.8 Hz, 3H), 2.60-2.55 (m, 2H), 2.17-1.90 (m, 11H), 1.87-1.71 (m, 6H), 1.58 (s, 4H), 1.52-1.32 (m, 5H).

### Example 111

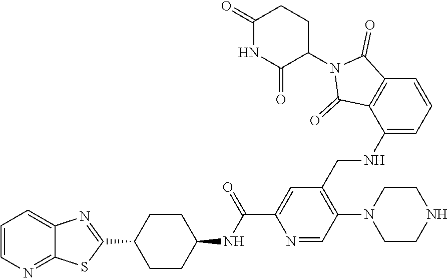 [SMILES: O=C1CCC(N2C(=O)c3cccc(NCc4cc(C(=O)N[C@H]5CC[C@H](c6nc7cccnc7s6)CC5)ncc4N4CCNCC4)c3C2=O)C(=O)N1]

### Preparation Steps

### Step 1

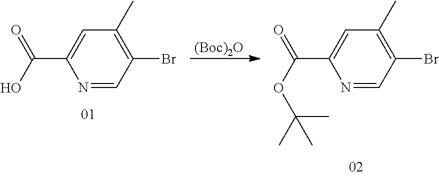

01 (1.00 g, 4.63 mmol) and 4-dimethylaminopyridine (56.6 mg, 0.463 mmol) were dissolved in anhydrous tetrahydrofuran (10 mL), and di-tert-butyl dicarbonate (2.02 g, 9.26 mmol) was added dropwise at 0° C. The reaction mixture was stirred at room temperature for 4 h. Water (60 mL) was added to the mixture, followed by extraction with ethyl acetate (20 mL×3). The organic phase was washed with saturated brine (60 mL), dried over anhydrous sodium sulfate, and filtered, and the filtrate was concentrated to dryness by rotary evaporation. The residue was purified through a flash silica gel column (petroleum ether:ethyl acetate=5:1) to give the target product as a yellow oil (1.16 g, 92.1% yield). LC-MS: 272.2 [M+H]⁺; 216.2 [M+H−56]⁺.

### Step 2

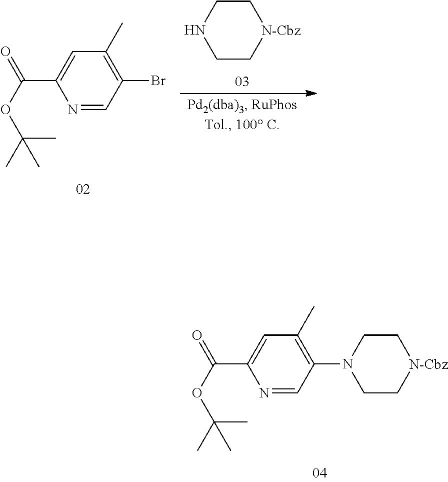

02 (1.06 g, 3.92 mmol), 1-benzyloxycarbonylpiperazine (863 mg, 3.92 mmol), cesium carbonate (3.83 g, 11.8 mmol), tris(dibenzylideneacetone)dipalladium (71.8 mg, 0.078 mmol) and 2-dicyclohexylphosphino-2,6-diisopropoxy-1,1-biphenyl (183 mg, 0.392 mmol) were dissolved in toluene (15 mL) at room temperature, and the reaction mixture was stirred in an argon atmosphere at 100° C. for 16 h and filtered. The filtrate was concentrated to dryness by rotary evaporation, and the mixture was passed through a silica gel column (petroleum ether:ethylacetate=2:1) to separate the target product as a yellow oil (1.31 g, 81.7% yield). LC-MS: 412.4 [M+H]⁺.

### Step 3

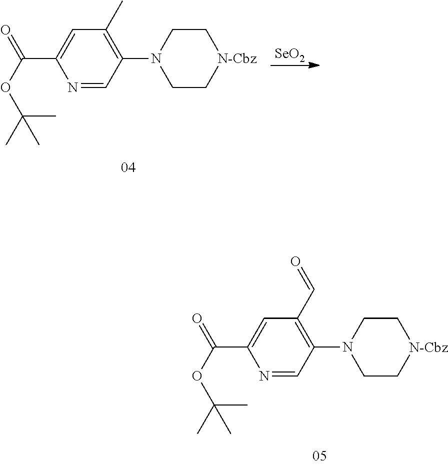

04 (1.30 g, 3.16 mmol) and selenium dioxide (1.75 g, 15.8 mmol) were dissolved in dioxane (15 mL) at room temperature, and the reaction mixture was stirred in an argon atmosphere at 110° C. for 16 h. The mixture was cooled to room temperature and then concentrated under reduced pressure to remove the solvent. Water (150 mL) was added, followed by extraction with ethyl acetate (40 mL×3). The organic phase was washed with saturated brine (80 mL), dried over anhydrous sodium sulfate, and filtered, and the filtrate was concentrated to dryness by rotary evaporation. The residue was passed through a silica gel column (petroleum ether:ethyl acetate=2:1) to separate the target product as a yellow solid (608 mg, 45.2% yield).

### Step 4

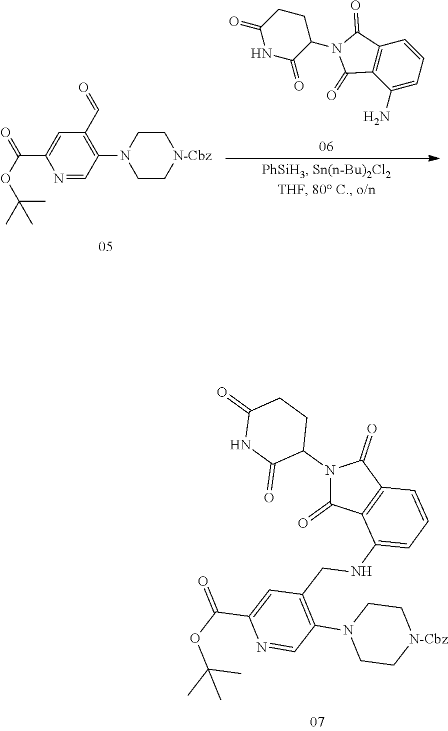

To a sealed tube were added 05 (562 mg, 1.32 mmol), pomalidomide (360 mg, 1.32 mmol), dibutyltin dichloride (480 mg, 1.58 mmol), phenylsilane (143 mg, 1.32 mmol) and anhydrous tetrahydrofuran (10 mL) at room temperature. The reaction mixture was stirred at 80° C. for 16 h, cooled to room temperature, and then filtered, and the filtrate was concentrated to dryness by rotary evaporation. The residue was purified by silica gel column chromatography (dichloromethane:methanol=20:1) and preparative thin-layer chromatography (dichloromethane:methanol=20:1) to separate the target product as a green solid (35 mg, 3.9% yield). LC-MS: 683.4 [M+H]⁺.

### Step 5

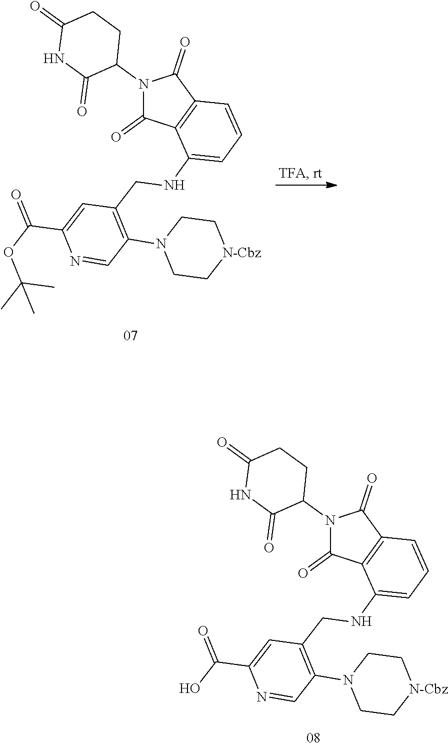

07 (35.0 mg, 0.051 mmol) was dissolved in dichloromethane (2 mL) at room temperature, and trifluoroacetic acid (0.4 mL) was added dropwise with stirring at room temperature. The reaction mixture was stirred at room temperature for 16 h. The mixture was concentrated to dryness by rotary evaporation to give the target product as a green solid (32 mg, crude). LC-MS: 627.3 [M+H]⁺.

### Step 6

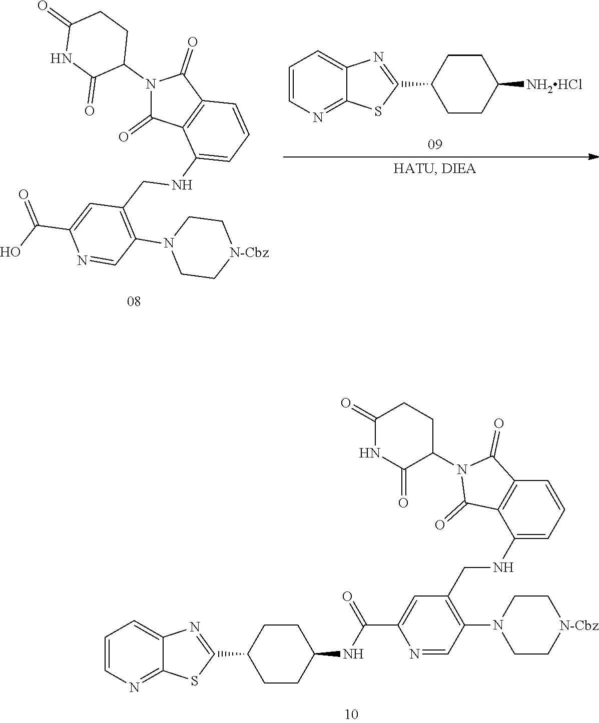

08 (32.0 mg, 0.051 mmol), 09 (13.8 mg, 0.051 mmol) and N,N-diisopropylethylamine (19.8 mg, 0.153 mmol) were mixed in anhydrous N,N-dimethylformamide (2 mL) at room temperature, and (7-azabenzotriazole)-tetramethyluronium hexafluorophosphate (29.3 mg, 0.077 mmol) was added with stirring at room temperature. The reaction mixture was stirred at room temperature for 2 h. The mixture was passed through a reversed-phase column (C18, 10-100% acetonitrile/water as mobile phase, 0.1% formic acid) to separate the target product as a green solid (14 mg, 32.6% yield). LC-MS: 842.4 [M+H]⁺.

### Step 7

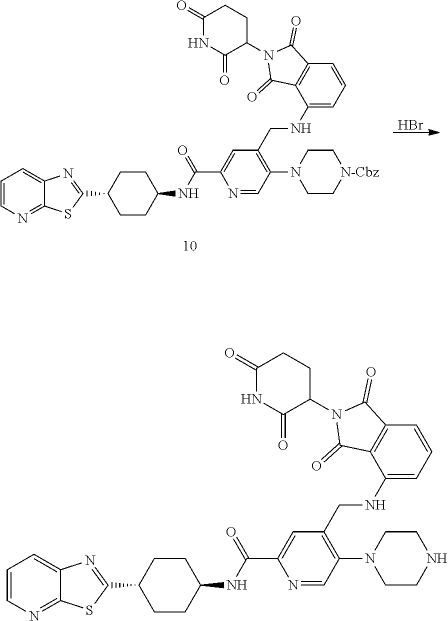

10 (12.0 mg, 0.014 mmol) was dissolved in hydrobromic acid (1 mL) at room temperature. The reaction mixture was stirred at room temperature for 30 min. The mixture was purified by preparative chromatography (C18, 5-100% acetonitrile/water as mobile phase, 0.1% formic acid) to separate the target product as a green solid (5.40 mg, 53.5% yield). LC-MS: 708.0 [M+H]⁺, ¹H NMR (400 MHz, DMSO-d6) δ 11.17 (s, 1H), 8.65-8.60 (m, 1H), 8.39 (td, J=17.1, 8.4 Hz, 3H), 8.26 (s, 1H), 8.08-8.03 (m, 1H), 7.65-7.58 (m, 2H), 7.57-7.50 (m, 1H), 7.11-7.05 (m, 1H), 6.84-6.77 (m, 1H), 5.14 (dd, J=12.8, 5.4 Hz, 1H), 4.64 (d, J=6.4 Hz, 2H), 3.92-3.84 (m, 1H), 3.20-3.08 (m, 9H), 2.99-2.91 (m, 1H), 2.69-2.58 (m, 2H), 2.26 (d, J=11.8 Hz, 2H), 2.19-1.94 (m, 4H), 1.83-1.74 (m, 2H), 1.73-1.61 (m, 2H).

### Example 112

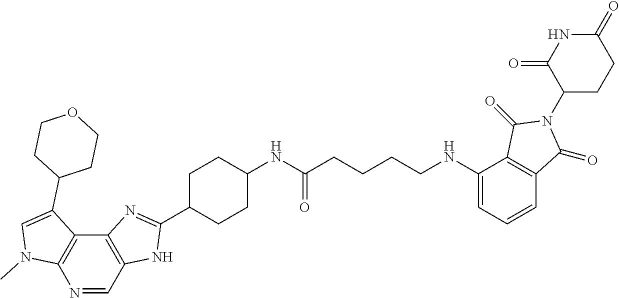 [SMILES: Cn1cc(C2CCOCC2)c2c3nc(C4CCC(NC(=O)CCCCNc5cccc6c5C(=O)N(C5CCC(=O)NC5=O)C6=O)CC4)[nH]c3cnc21]

### Preparation Steps

### Step 1

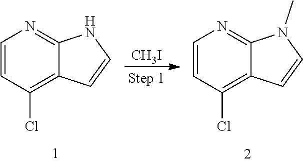

In a 500 mL single-neck flask, 1 (10 g, 65.5 mmol) was dissolved in DMF (100 mL), and NaH (60%, 5.3 g, 131 mmol) was added with cooling in an ice bath. The mixture was stirred for 5 min, followed by addition of iodomethane (10.2 g, 72.1 mmol). The mixture was stirred at room temperature for 1 h, quenched with ice water, and extracted with ethyl acetate. The organic phase was washed once with saturated brine, dried over anhydrous sodium sulfate, concentrated, and purified by column chromatography to give the target product (7.9 g, 72%). LC-MS: 167 [M+H]⁺.

### Step 2

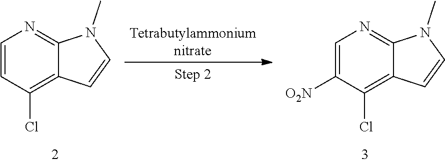

In a 500 mL single-neck flask, 2 (7.9 g, 47.2 mmol) was dissolved in DMF (100 mL), and the resulting solution was cooled to 0° C. Tetrabutylammonium nitrate (21.7 g, 71.1 mmol) and trifluoroacetic anhydride (14.9 g, 71.7 mmol) were added dropwise slowly to the system. The system was reacted at room temperature for 20 h, quenched with saturated sodium bicarbonate solution, and extracted with dichloromethane. The organic phase was washed once with saturated brine, dried over anhydrous sodium sulfate, concentrated, and purified by column chromatography to give the target product (3.8 g, 38%). LC-MS: 212 [M+H]⁺.

### Step 3

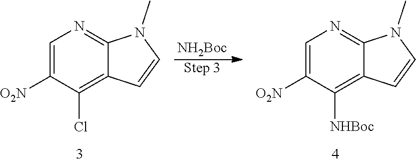

In a 250 mL single-neck flask, 3 (3.8 g, 18.0 mmol), NH₂Boc (3.2 g, 24.9 mmol), cesium carbonate (11.7 g, 35.9 mmol), Xantphos (1.6 g, 2.7 mmol) and Pd₂(dba)₃ (822 mg, 0.9 mmol) were dissolved in 1,4-dioxane (50 mL), and the mixture was reacted in a nitrogen atmosphere at 110° C. for 16 h and filtered under vacuum. The filtrate was quenched with water and extracted by ethyl acetate, and the organic phase was concentrated under reduced pressure and purified by column chromatography to give the target product (2.5 g, 48%). LC-MS: 293 [M+H]⁺.

### Step 4

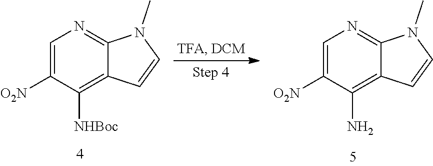 [SMILES: Cn1ccc2c(NC(=O)OC(C)(C)C)c([N+](=O)[O-])cnc21 ; Cn1ccc2c(N)c([N+](=O)[O-])cnc21]

In a 100 mL single-neck flask, 4 (2.5 g, 8.6 mmol) was dissolved in DCM (20 mL), and trifluoroacetic acid (4 mL) was added. The mixture was reacted at room temperature for 16 h, concentrated under reduced pressure, quenched with saturated sodium bicarbonate solution, and extracted with dichloromethane. The organic phase was concentrated and purified by column chromatography to give the target product (1.4 g, 85%). LC-MS: 193 [M+H]⁺.

### Step 5

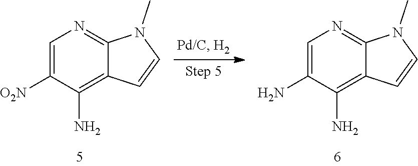

In a 100 mL single-neck flask, 5 (1.4 g, 7.3 mmol) was dissolved in methanol (20 mL), and palladium on carbon (300 mg) was added. The mixture was reacted in a hydrogen atmosphere at room temperature for 16 h and filtered, and the filtrate was concentrated under reduced pressure and dried to give the target product (1.1 g, 93%). LC-MS: 163 [M+H]⁺.

### Step 6

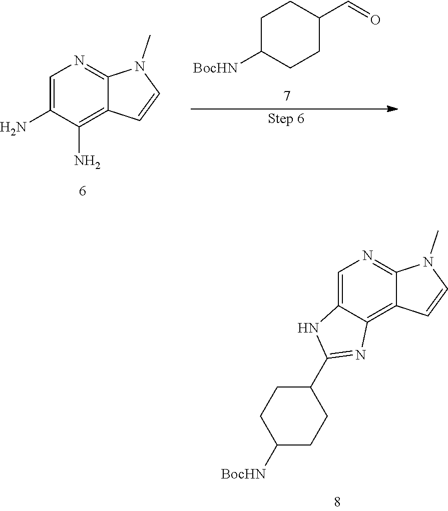 [SMILES: Cn1ccc2c(N)c(N)cnc21 ; CC(C)(C)OC(=O)NC1CCC(C=O)CC1 ; "Step 6" ; Cn1ccc2c3nc(C4CCC(NC(=O)OC(C)(C)C)CC4)[nH]c3cnc21]

In a 100 mL single-neck flask, 6 (1.1 g, 6.8 mmol) and 7 (2.0 g, 8.8 mmol) were dissolved in acetic acid/methanol (5 mL/10 mL), and the mixture was reacted in an oxygen atmosphere at room temperature for 16 h, concentrated under reduced pressure, quenched with water, and extracted with dichloromethane. The organic phase was washed once with water, dried over anhydrous sodium sulfate, concentrated under reduced pressure, and purified by column chromatography to give the target product (1.0 g, 40%). LC-MS: 370 [M+H]⁺.

### Step 7

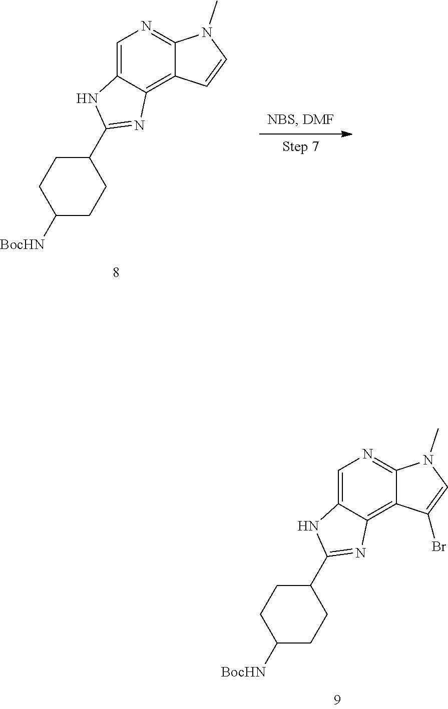

In a 100 mL single-neck flask, 8 (1.0 g, 2.7 mmol) was dissolved in DMF (20 mL), and NBS (578 mg, 3.3 mmol) was added. The mixture was reacted at room temperature for 16 h, quenched with water, and extracted with ethyl acetate. The organic phase was washed once with saturated brine, dried over anhydrous sodium sulfate, concentrated under reduced pressure, and purified by column chromatography to give the target product (620 mg, 51%). LC-MS: 448 [M+H]⁺.

### Step 8

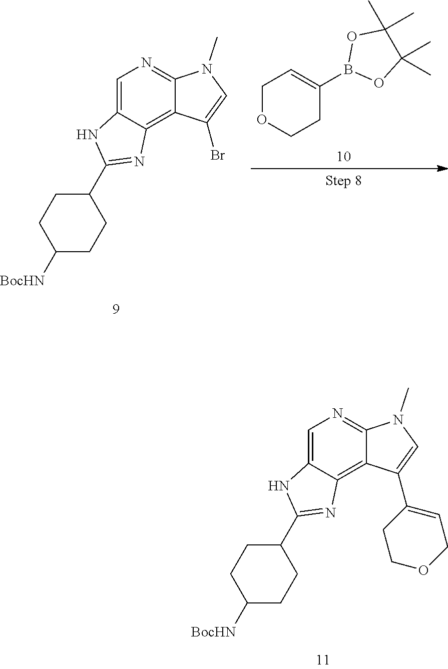

In a 100 mL single-neck flask, 9 (620 mg, 1.38 mmol), 10 (436 mg, 2.07 mmol), Pd(dppf)Cl₂-DCM (56 mg, 0.07 mmol) and potassium carbonate (573 mg, 4.15 mmol) were dissolved in 1,4-dioxane/water (10 mL/2 mL), and the mixture was reacted in a nitrogen atmosphere at 80° C. for 16 h, concentrated under reduced pressure, and purified by column chromatography to give the target product (340 mg, 54%). LC-MS: 452 [M+H]⁺.

### Step 9

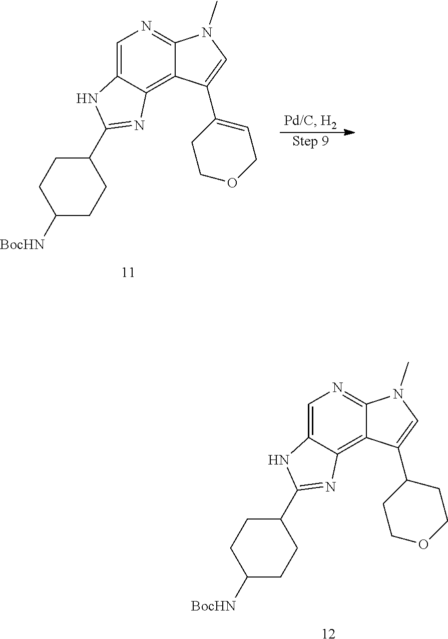 [SMILES: Cn1cc(C2=CCOCC2)c2c3nc(C4CCC(NC(=O)OC(C)(C)C)CC4)[nH]c3cnc21 ; "Step 9" ; "Pd/C, H2" ; Cn1cc(C2CCOCC2)c2c3nc(C4CCC(NC(=O)OC(C)(C)C)CC4)[nH]c3cnc21 ; "12"]

In a 100 mL single-neck flask, 11 (340 mg, 0.75 mmol) was dissolved in methanol (10 mL), and palladium on carbon (100 mg) was added. The mixture was reacted in a hydrogen atmosphere at room temperature for 16 h and filtered, and the filtrate was concentrated under reduced pressure and dried to give the target product (210 mg, 62%). LC-MS: 454 [M+H]⁺.

### Step 10

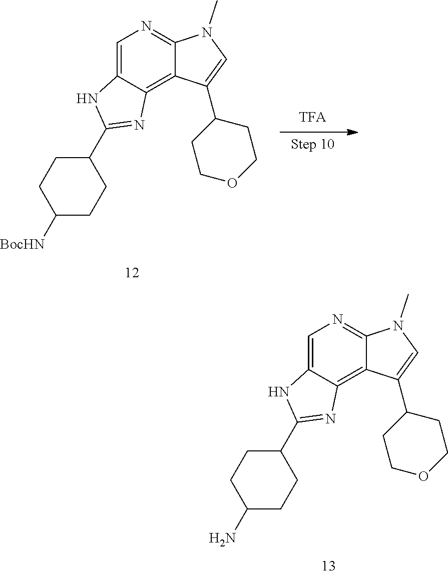 [SMILES: Cn1cc(C2CCOCC2)c2c3nc(C4CCC(NC(=O)OC(C)(C)C)CC4)[nH]c3cnc21 ; "Step 10" ; Cn1cc(C2CCOCC2)c2c3nc(C4CCC(N)CC4)[nH]c3cnc21 ; "TFA"]

In a 100 mL single-neck flask, 12 (210 mg, 0.46 mmol) was dissolved in dichloromethane (5 mL), and trifluoroacetic acid (1 mL) was added. The mixture was reacted at room temperature for 16 h, concentrated under reduced pressure, and purified by preparative liquid chromatography to give the target product (64 mg, 39%). LC-MS: 354 [M+H]⁺.

### Step 11

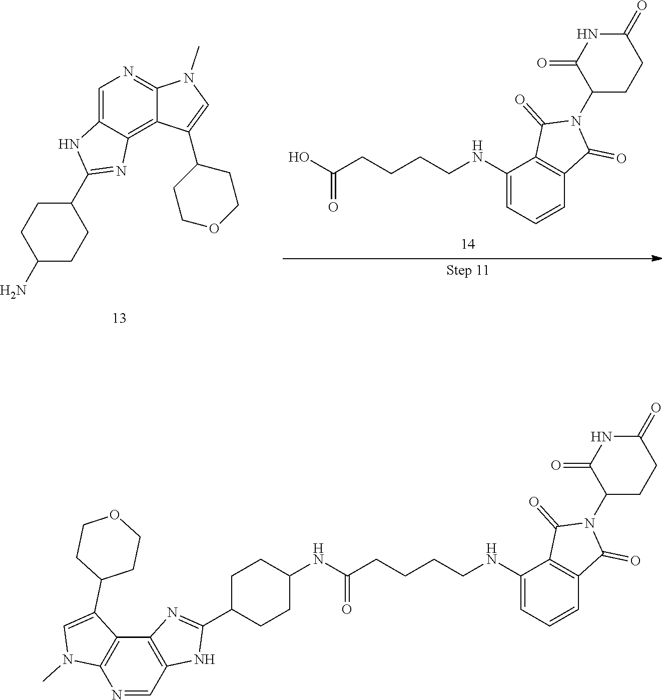 [SMILES: Cn1cc(C2CCOCC2)c2c3nc(C4CCC(N)CC4)[nH]c3cnc21 ; O=C(O)CCCCNc1cccc2c1C(=O)N(C1CCC(=O)NC1=O)C2=O ; Cn1cc(C2CCOCC2)c2c3nc(C4CCC(NC(=O)CCCCNc5cccc6c5C(=O)N(C5CCC(=O)NC5=O)C6=O)CC4)[nH]c3cnc21]

In a 25 mL single-neck flask, 13 (64 mg, 0.18 mmol), 14 (68 mg, 0.18 mmol), HATU (83 mg, 0.22 mmol) and DIEA (70 mg, 0.54 mmol) were dissolved in DMF (2 mL), and the mixture was reacted at room temperature for 16 h, concentrated under reduced pressure, and purified by preparative liquid chromatography to give the bright yellow target product (4.3 mg, 3.4%). LC-MS: 709 [M+H]⁺, 1H NMR (400 MHz, DMSO) δ 11.23 (s, 1H), 11.11 (s, 1H), 8.45 (s, 1H), 7.82 (d, J=7.6 Hz, 1H), 7.59 (dd, J=8.4, 7.2 Hz, 1H), 7.15-6.97 (m, 3H), 6.60 (t, J=6.0 Hz, 1H), 5.06 (dd, J=12.9, 5.4 Hz, 1H), 3.98 (dd, J=10.8, 3.0 Hz, 2H), 3.89 (d, J=11.9 Hz, 3H), 3.66-3.60 (m, 1H), 3.57-3.50 (m, 2H), 3.26-3.15 (m, 2H), 3.04-2.82 (m, 3H), 2.58 (dd, J=19.4, 5.9 Hz, 3H), 2.14 (d, J=14.0 Hz, 3H), 2.02 (dd, J=16.5, 9.1 Hz, 3H), 1.90 (t, J=11.7 Hz, 2H), 1.86-1.77 (m, 2H), 1.77-1.66 (m, 2H), 1.58 (s, 4H), 1.37 (dd, J=23.2, 10.7 Hz, 2H).

### Example 113

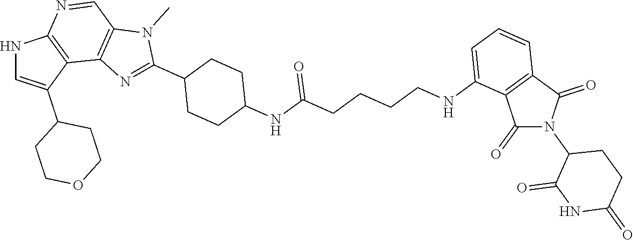 [SMILES: Cn1c(C2CCC(NC(=O)CCCCNc3cccc4c3C(=O)N(C3CCC(=O)NC3=O)C4=O)CC2)nc2c3c(C4CCOCC4)c[nH]c3ncc21]

### Preparation Steps

### Step 1

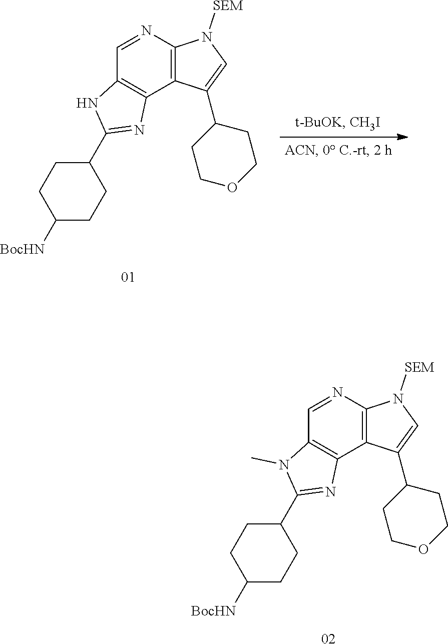 [SMILES: CC(C)(C)OC(=O)NC1CCC(c2nc3c(cnc4c3c(C3CCOCC3)cn4COCC[Si](C)(C)C)[nH]2)CC1 ; Cn1c(C2CCC(NC(=O)OC(C)(C)C)CC2)nc2c3c(C4CCOCC4)cn(COCC[Si](C)(C)C)c3ncc21 ; "02"]

In a 50 mL single-neck flask, 01 (140 mg, 0.246 mmol) was dissolved in acetonitrile (5 mL), and potassium tert-butoxide (55 mg, 0.491 mmol) was added at 0° C. After the mixture was stirred for 0.5 h, iodomethane (174 mg, 1.23 mmol) was added. The mixture was reacted at room temperature for 2 h and extracted with ethyl acetate (20 mL) and water (10 mL). The organic phase was dried and filtered, and the filtrate was concentrated to dryness by rotary evaporation. The residue was purified by preparative thin-layer chromatography to give the target product as a yellow solid (113 mg, 78.77% yield). LC-MS: 584 [M+H]⁺.

### Step 2

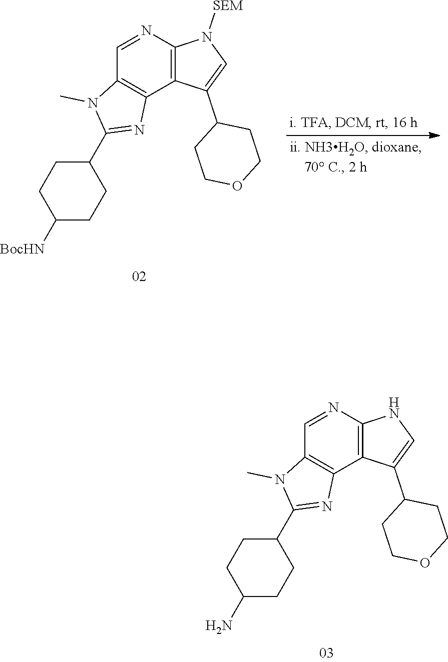 [SMILES: Cn1c(C2CCC(NC(=O)OC(C)(C)C)CC2)nc2c3c(C4CCOCC4)cn(COCC[Si](C)(C)C)c3ncc21 ; Cn1c(C2CCC(N)CC2)nc2c3c(C4CCOCC4)c[nH]c3ncc21]

In a 50 mL single-neck flask, 02 (113 mg, 0.194 mmol) was dissolved in dichloromethane (5 mL), and trifluoroacetic acid (0.5 mL) was added. The reaction mixture was stirred at room temperature for 16 h. After the reaction mixture was concentrated to dryness by rotary evaporation, 1,4-dioxane (5 mL) and ammonia solution (1 mL) were added. The reaction mixture was stirred at 70° C. for 3 h and concentrated to dryness by rotary evaporation, and the residue was purified by preparative reversed-phase liquid chromatography to give the target product as a white solid (44 mg, 64.32% yield). LC-MS: 354 [M+H]⁺.

### Step 3

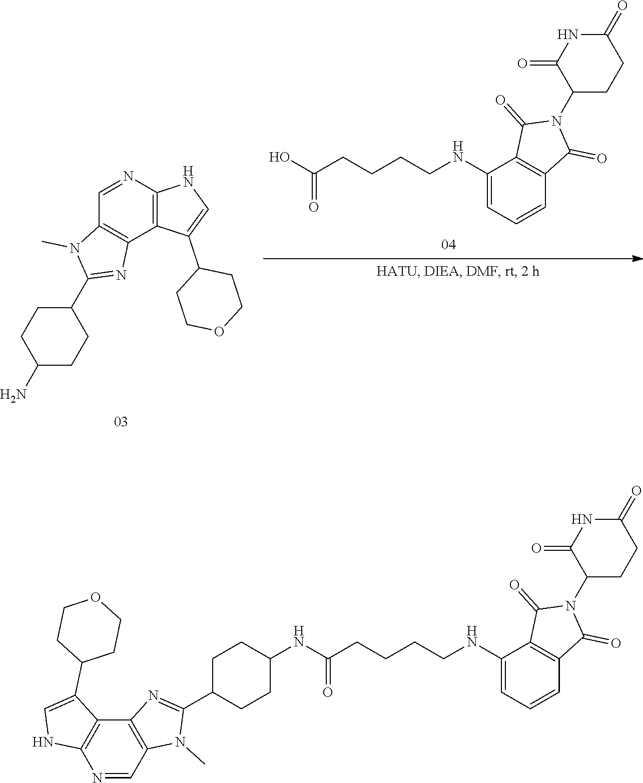 [SMILES: Cn1c(C2CCC(N)CC2)nc2c3c(C4CCOCC4)c[nH]c3ncc21 ; O=C(O)CCCCNc1cccc2c1C(=O)N(C1CCC(=O)NC1=O)C2=O ; Cn1c(C2CCC(NC(=O)CCCCNc3cccc4c3C(=O)N(C3CCC(=O)NC3=O)C4=O)CC2)nc2c3c(C4CCOCC4)c[nH]c3ncc21]

In a 50 mL single-neck flask, 03 (25 mg, 0.0707 mmol) was dissolved in N,N-dimethylformamide (2 mL), and HATU (40 mg, 0.106 mmol), N,N-diisopropylethylamine (27 mg, 0.212 mmol) and 04 (25 mg, 0.0707 mmol) were added. The reaction mixture was stirred at room temperature for 16 h and extracted with ethyl acetate (10 mL) and water (5 mL×3). The organic phases were combined, dried, and concentrated to dryness by rotary evaporation, and the residue was purified by preparative reversed-phase liquid chromatography to give the target product as a yellow solid (4.98 mg, 9.93% yield). LC-MS: 709.2 [M+H]⁺.

HNMR: ¹H NMR (400 MHz, DMSO-d₆) δ 11.22 (s, 1H), 11.08 (s, 1H), 8.45 (s, 1H), 7.79 (d, J=7.7 Hz, 1H), 7.63-7.55 (m, 1H), 7.13-7.07 (m, 2H), 7.03 (d, J=7.0 Hz, 1H), 6.58 (t, J=5.8 Hz, 1H), 5.05 (dd, J=12.8, 5.3 Hz, 1H), 3.98 (dd, J=11.0, 3.2 Hz, 2H), 3.88 (s, 3H), 3.68-3.59 (m, 1H), 3.54 (t, J=10.9 Hz, 2H), 3.22 (t, J=11.8 Hz, 1H), 3.02-2.82 (m, 2H), 2.64-2.52 (m, 2H), 2.14 (d, J=12.5 Hz, 4H), 2.01-1.78 (m, 11H), 1.59 (s, 4H), 1.43-1.21 (m, 4H).

### Example 114

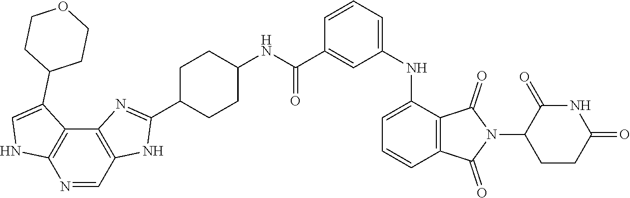 [SMILES: O=C1CCC(N2C(=O)c3cccc(Nc4cccc(C(=O)NC5CCC(c6nc7c(cnc8[nH]cc(C9CCOCC9)c87)[nH]6)CC5)c4)c3C2=O)C(=O)N1]

### Preparation Steps

### Step 1

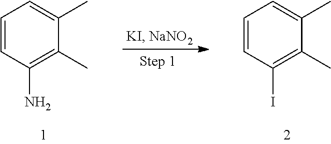

A solution of HCl (32%, 25 mL, 0.22 mol) in H₂O (50 mL) was added dropwise to 2,3-dimethylaniline (10 g, 0.082 mol) maintained at 90° C. and mixed well by stirring and then the mixture was cooled and maintained at −10° C. Then a solution of NaNO₂ (6.1 g, 0.09 mol) in H₂O (50 mL) was added dropwise to the mixture, and the mixture was stirred for 30 min, followed by addition of KI (15.2 g, 0.092 mol). The mixture was stirred at 55° C. for 5 min, then stirred at room temperature overnight, and extracted with ethyl acetate (50 mL×3). The organic phase was washed with 1N sodium hydroxide solution, dried over anhydrous sodium sulfate, and concentrated to dryness by rotary evaporation to give the product as a brown liquid (6.9 g, 36.24%).

### Step 2

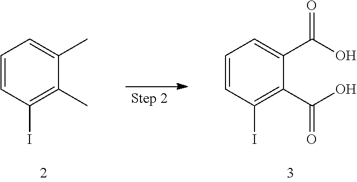

KMnO₄ (13.8 g, 89.1 mmol) was added to a stirred mixture of compound 2 (6.9 g, 29.7 mmol) and NaOH (0.54 g, 13.5 mmol) in H₂O (220 mL), and the resulting mixture was maintained at 65° C. The resulting mixture was heated at reflux for 2 days and filtered. The filtrate was acidified with hydrochloric acid and extracted with ethyl acetate (3×500 mL), and the organic phase was washed with saturated sodium chloride, dried over anhydrous sodium sulfate, and concentrated to dryness by rotary evaporation to give a brown solid (5.1 g, 58.81%). MS: 291 [M−H]⁻.

### Step 3

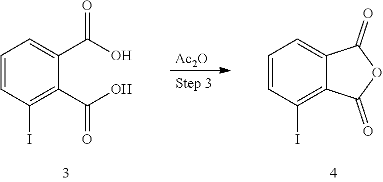 [SMILES: "Ac2O" ; O=C(O)c1cccc(I)c1C(=O)O ; O=C1OC(=O)c2c(I)cccc21]

Acetic anhydride (25 mL) was added to a solution of 3-iodophthalic acid (5.1 g, 17.1 mmol) in THF (75 mL) at room temperature. The reaction mixture was heated to 85° C. and stirred for 6 h, then cooled to room temperature, and concentrated to give the product as a yellow solid (3.3 g, 70.43%). MS: 275 [M+H]⁺.

### Step 4

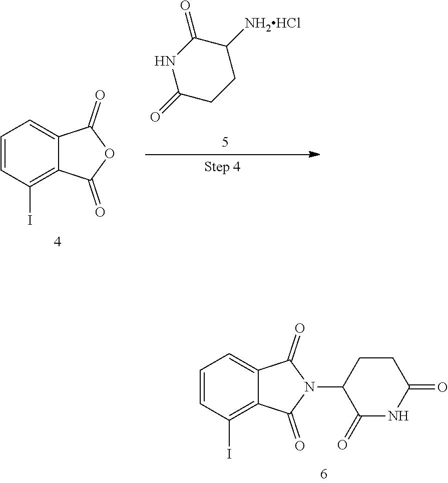

Compound 5 (1.98 g, 12 mmol) was added to a solution of compound 4 (3.30 g, 12 mmol) in CH₃COOH (100 mL). The mixture was refluxed for 12 h, then diluted with EtOAc (100 mL), washed with saturated HCl solution (1N, 50 mL), dried over Na₂SO₄, and concentrated under reduced pressure. The residue was purified by column chromatography on silica gel (dichloromethane:methanol=50:1) to give the compound 8 as a white solid (2.6 g, 56.67%). MS: 385 [M+H]⁺.

### Step 5

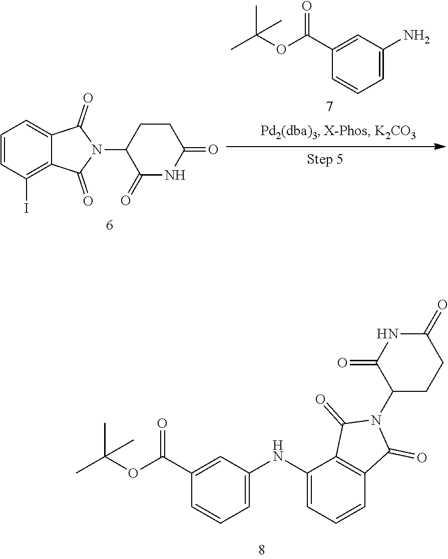 [SMILES: CC(C)(C)OC(=O)c1cccc(N)c1 ; O=C1CCC(N2C(=O)c3cccc(I)c3C2=O)C(=O)N1 ; CC(C)(C)OC(=O)c1cccc(Nc2cccc3c2C(=O)N(C2CCC(=O)NC2=O)C3=O)c1]

Compound 6 (2.6 g, 6.8 mmol), compound 7 (1.3 g, 6.8 mmol), Pd₂(dba)₃ (622 mg, 0.68 mmol), X-Phos (485 mg, 1.02 mmol) and K₂CO₃ (1876 mg, 13.6 mmol) were added to a three-necked flask containing toluene in a nitrogen atmosphere. The reaction mixture was stirred at 100° C. for 18 h and filtered, and the filtrate was collected, dried, and purified by column chromatography (ethyl acetate:petroleum ether=1:3) to give the product as a yellow solid (310 mg, 10.15%). MS: 450 [M+H]⁺.

### Step 6

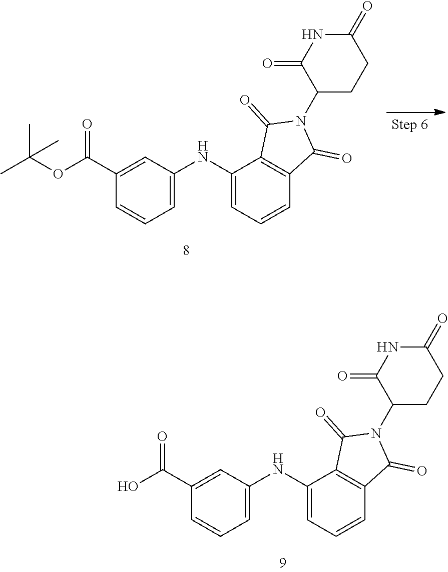 [SMILES: CC(C)(C)OC(=O)c1cccc(Nc2cccc3c2C(=O)N(C2CCC(=O)NC2=O)C3=O)c1 ; O=C1CCC(N2C(=O)c3cccc(Nc4cccc(C(=O)O)c4)c3C2=O)C(=O)N1]

Compound 8 (270 mg, 0.6 mmol) was added to 10% wt TFA/DCM. The mixture was stirred at room temperature for 2 h and extracted with dichloromethane (10 mL×3), and the aqueous phase was retained, adjusted to pH 7 with sodium bicarbonate, and extracted with ethyl acetate. The organic phase was dried over anhydrous sodium sulfate and concentrated to dryness by rotary evaporation to give the product (126 mg, 53.29%). MS: 394 [M+H]⁺.

### Step 7

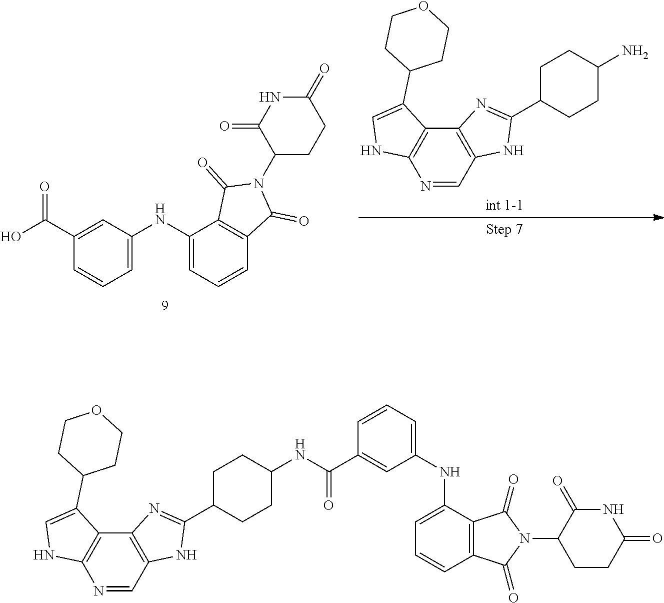 [SMILES: O=C1CCC(N2C(=O)c3cccc(Nc4cccc(C(=O)O)c4)c3C2=O)C(=O)N1 ; NC1CCC(c2nc3c(cnc4[nH]cc(C5CCOCC5)c43)[nH]2)CC1 ; O=C1CCC(N2C(=O)c3cccc(Nc4cccc(C(=O)NC5CCC(c6nc7c(cnc8[nH]cc(C9CCOCC9)c87)[nH]6)CC5)c4)c3C2=O)C(=O)N1]

Compound 9 (126 mg, 0.31 mmol), int1-1 (105 mg, 0.30 mmol), HATU (177 mg, 0.47 mmol) and DIEA (122 mg, 0.94 mmol) were added to a flask containing dichloromethane. The mixture was stirred at room temperature for 18 h and extracted with dichloromethane (10 mL×3), and the organic phase was washed with saturated brine, dried over anhydrous sodium sulfate, and concentrated to dryness by rotary evaporation. The residue was purified by preparative high performance liquid chromatography to give the product (3 mg, 1.35%). MS: 715 [M+H]⁺, 1H NMR (400 MHz, DMSO) δ 12.30 (d, J=89.0 Hz, 1H), 11.42-11.10 (m, 2H), 8.58 (s, 2H), 8.38 (dd, J=27.4, 17.7 Hz, 1H), 8.14 (s, 1H), 7.81 (s, 1H), 7.74-7.55 (m, 2H), 7.47 (dd, J=14.0, 7.2 Hz, 2H), 7.30 (d, J=7.0 Hz, 1H), 7.12 (d, J=12.2 Hz, 1H), 5.14 (dd, J=12.8, 5.3 Hz, 1H), 3.97 (s, 1H), 3.91 (s, 1H), 3.64 (t, J=11.3 Hz, 1H), 3.54 (t, J=11.4 Hz, 1H), 3.02-2.82 (m, 1H), 2.61 (dd, J=36.5, 15.4 Hz, 2H), 2.29-1.98 (m, 3H), 1.98-1.70 (m, 2H), 1.70-1.37 (m, 2H), 1.19 (d, J=34.6 Hz, 1H).

### Example 115

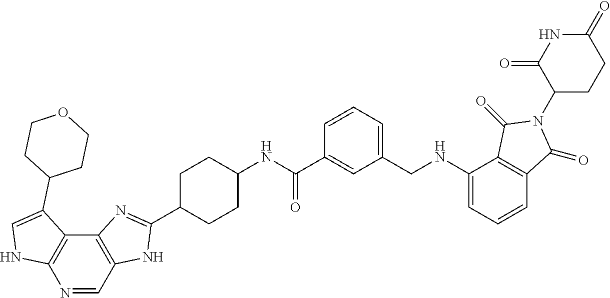 [SMILES: O=C1CCC(N2C(=O)c3cccc(NCc4cccc(C(=O)NC5CCC(c6nc7c(cnc8[nH]cc(C9CCOCC9)c87)[nH]6)CC5)c4)c3C2=O)C(=O)N1]

### Preparation Steps

### Step 1

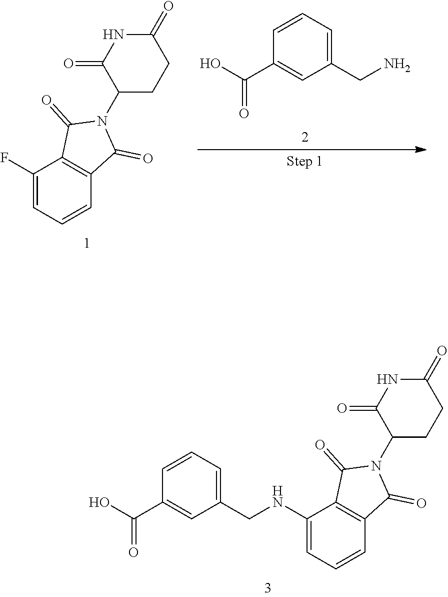 [SMILES: O=C1CCC(N2C(=O)c3cccc(F)c3C2=O)C(=O)N1 ; NCc1cccc(C(=O)O)c1 ; O=C1CCC(N2C(=O)c3cccc(NCc4cccc(C(=O)O)c4)c3C2=O)C(=O)N1]

1 (500 mg, 1.81 mmol), 2 (330. mg, 2.17 mmol) and N-methylpyrrolidone (5 mL) were added to a 100 mL single-neck flask and mixed well by stirring, and DIEA (701.7, 5.43 mmol) was added. The mixture was reacted at 110° C. for 18 h and concentrated to dryness by rotary evaporation, and the residue was purified by column chromatography to give the target product as a yellowish-green solid (320 mg, 43.4%). LC-MS: 408 [M+H]⁺.

### Step 2

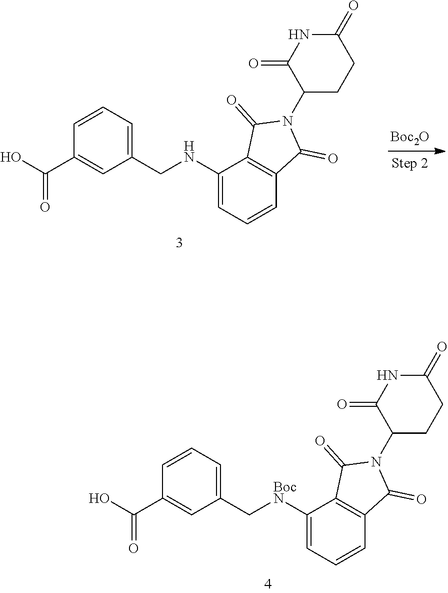 [SMILES: "Boc2O" ; O=C1CCC(N2C(=O)c3cccc(NCc4cccc(C(=O)O)c4)c3C2=O)C(=O)N1 ; CC(C)(C)OC(=O)N(Cc1cccc(C(=O)O)c1)c1cccc2c1C(=O)N(C1CCC(=O)NC1=O)C2=O]

In a 100 mL single-neck flask, 3 (320 mg, 0.786 mmol) was dissolved in THF (4 mL), and Boc₂O (205.6 mg, 0.942 mmol) and DMAP (3.9 mg, 0.032 mmol) were added. The mixture was reacted at room temperature for 16 h and concentrated to dryness by rotary evaporation, and the residue was purified by column chromatography to give the target product as a yellowish-green solid (265 mg, 66.5%). LC-MS: 508 [M+H]⁺.

### Step 3

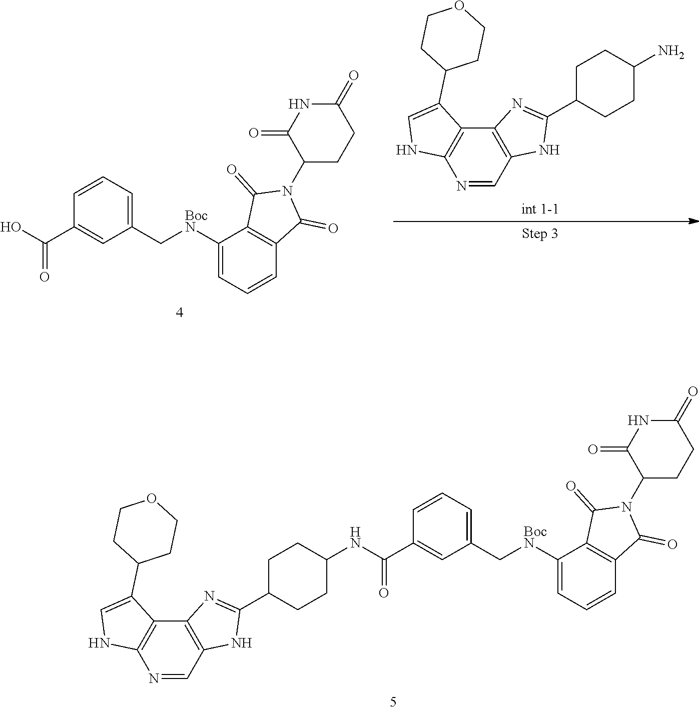 [SMILES: CC(C)(C)OC(=O)N(Cc1cccc(C(=O)O)c1)c1cccc2c1C(=O)N(C1CCC(=O)NC1=O)C2=O ; NC1CCC(c2nc3c(cnc4[nH]cc(C5CCOCC5)c43)[nH]2)CC1 ; CC(C)(C)OC(=O)N(Cc1cccc(C(=O)NC2CCC(c3nc4c(cnc5[nH]cc(C6CCOCC6)c54)[nH]3)CC2)c1)c1cccc2c1C(=O)N(C1CCC(=O)NC1=O)C2=O]

In a 100 mL single-neck flask, 4 (265 mg, 0.530 mmol) and int1-1 (214 mg, 0.630 mmol) were dissolved in DMF (4 mL) and mixed well by stirring, and HATU (298.5 mg, 0.795 mmol) and DIEA (205.5 mg, 1.59 mmol) were added. The mixture was reacted at room temperature for 18 h and concentrated to dryness by rotary evaporation, and the residue was purified by column chromatography to give the target product as a yellowish-green solid (74 mg, 17.0%). LC-MS: 829 [M+H]⁺.

### Step 4

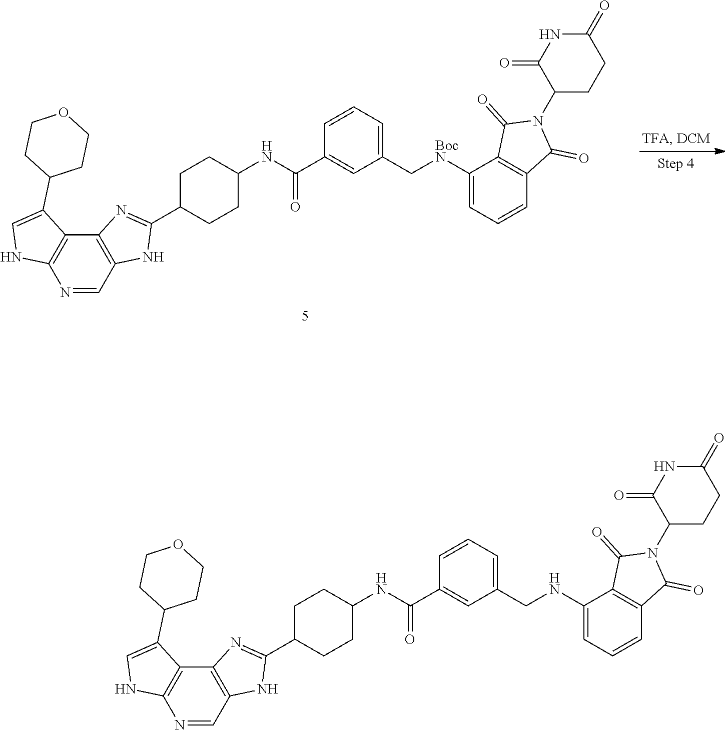 [SMILES: CC(C)(C)OC(=O)N(Cc1cccc(C(=O)NC2CCC(c3nc4c(cnc5[nH]cc(C6CCOCC6)c54)[nH]3)CC2)c1)c1cccc2c1C(=O)N(C1CCC(=O)NC1=O)C2=O ; O=C1CCC(N2C(=O)c3cccc(NCc4cccc(C(=O)NC5CCC(c6nc7c(cnc8[nH]cc(C9CCOCC9)c87)[nH]6)CC5)c4)c3C2=O)C(=O)N1]

In a 100 mL single-neck flask, 5 (74 mg, 0.0892 mmol) was dissolved in DCM (2 mL), and TFA (0.4 mL) was added. The mixture was reacted at room temperature for 16 h and concentrated to dryness by rotary evaporation, and the residue was purified by preparative high performance liquid chromatography to give the target product (3.5 mg, 4.78% yield). LC-MS: 729 [M+H]⁺, 1H NMR (400 MHz, DMSO) δ 12.07 (s, 1H), 11.12 (s, 1H), 8.72 (s, 1H), 8.38 (d, J=7.9 Hz, 1H), 7.88 (s, 1H), 7.75 (d, J=7.8 Hz, 1H), 7.52 (dd, J=10.7, 4.9 Hz, 2H), 7.44 (t, J=5.7 Hz, 2H), 7.32 (t, J=6.1 Hz, 1H), 7.04 (d, J=7.0 Hz, 1H), 6.96 (d, J=8.6 Hz, 1H), 5.09 (dd, J=12.8, 5.3 Hz, 1H), 4.63 (d, J=5.9 Hz, 2H), 3.98 (dd, J=10.5, 3.3 Hz, 5H), 2.88 (d, J=11.7 Hz, 1H), 2.24 (s, 1H), 2.07-1.94 (m, 6H), 1.86-1.72 (m, 4H), 1.62-1.53 (m, 2H), 1.23 (s, 4H).

### Example 116

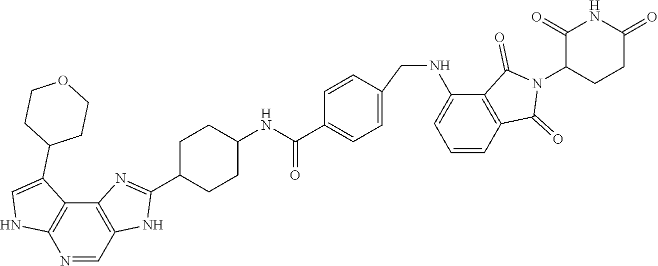 [SMILES: O=C1CCC(N2C(=O)c3cccc(NCc4ccc(C(=O)NC5CCC(c6nc7c(cnc8[nH]cc(C9CCOCC9)c87)[nH]6)CC5)cc4)c3C2=O)C(=O)N1]

### Preparation Steps

### Step 1

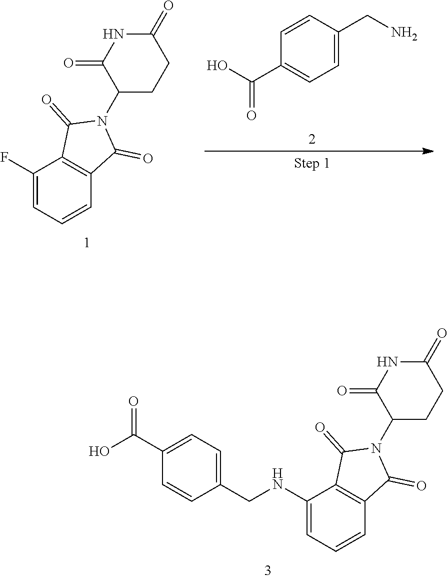 [SMILES: O=C1CCC(N2C(=O)c3cccc(F)c3C2=O)C(=O)N1 ; NCc1ccc(C(=O)O)cc1 ; O=C1CCC(N2C(=O)c3cccc(NCc4ccc(C(=O)O)cc4)c3C2=O)C(=O)N1]

1 (500 mg, 1.81 mmol), 2 (270 mg, 2.17 mmol) and N-methylpyrrolidone (5 mL) were added to a 100 mL single-neck flask and mixed well by stirring, and DIEA (1.17 mg, 9.05 mmol) was added. The mixture was reacted at 110° C. for 18 h and concentrated to dryness by rotary evaporation, and the residue was purified by column chromatography to give the target product as a yellowish-green solid (345 mg, 46.8%). LC-MS: 408 [M+H]⁺.

### Step 2

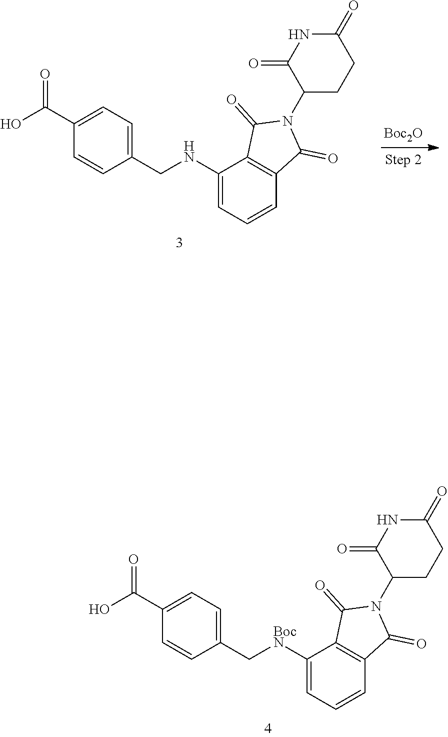 [SMILES: "Boc2O" ; O=C1CCC(N2C(=O)c3cccc(NCc4ccc(C(=O)O)cc4)c3C2=O)C(=O)N1 ; CC(C)(C)OC(=O)N(Cc1ccc(C(=O)O)cc1)c1cccc2c1C(=O)N(C1CCC(=O)NC1=O)C2=O]

In a 100 mL single-neck flask, 3 (345 mg, 0.852 mmol) was dissolved in THF (4 mL), and Boc₂O (222.8 mg, 1.02 mmol) and DMAP (4.2 mg, 0.0345 mmol) were added. The mixture was reacted at room temperature for 16 h and concentrated to dryness by rotary evaporation, and the residue was purified by column chromatography to give the target product as a yellowish-green solid (274 mg, 63.8%). LC-MS: 508 [M+H]⁺.

### Step 3

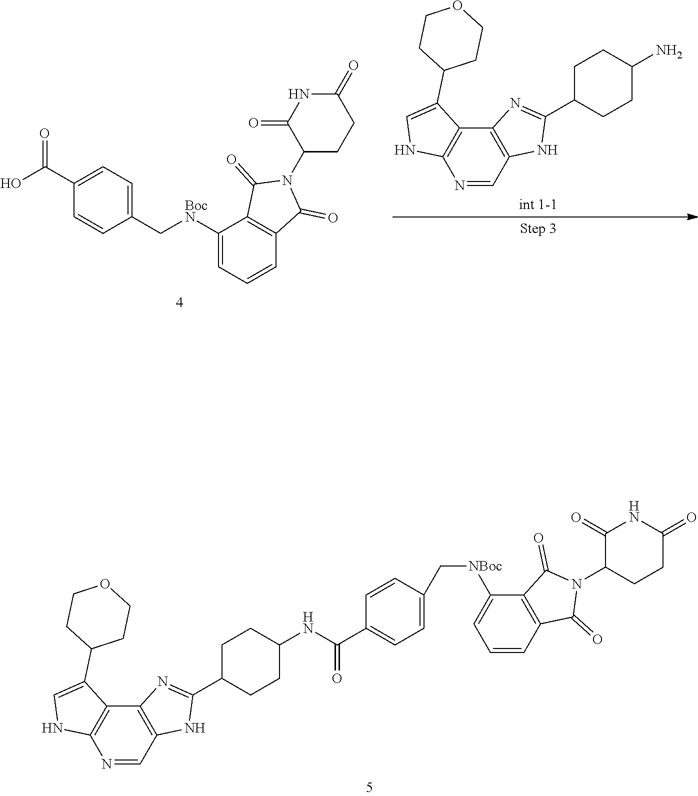 [SMILES: CC(C)(C)OC(=O)N(Cc1ccc(C(=O)O)cc1)c1cccc2c1C(=O)N(C1CCC(=O)NC1=O)C2=O ; NC1CCC(c2nc3c(cnc4[nH]cc(C5CCOCC5)c43)[nH]2)CC1 ; CC(C)(C)OC(=O)N(Cc1ccc(C(=O)NC2CCC(c3nc4c(cnc5[nH]cc(C6CCOCC6)c54)[nH]3)CC2)cc1)c1cccc2c1C(=O)N(C1CCC(=O)NC1=O)C2=O]

In a 100 mL single-neck flask, 4 (274 mg, 0.54 mmol) and int1-1 (218 mg, 0.642 mmol) were dissolved in DMF (2 mL) and mixed well by stirring, and HATU (308 mg, 0.81 mmol) and DIEA (209.5 mg, 1.62 mmol) were added. The mixture was reacted at room temperature for 18 h and concentrated to dryness by rotary evaporation, and the residue was purified by column chromatography to give the target product as a yellowish-green solid (82 mg, 18.3%). LC-MS: 829 [M+H]⁺.

### Step 4

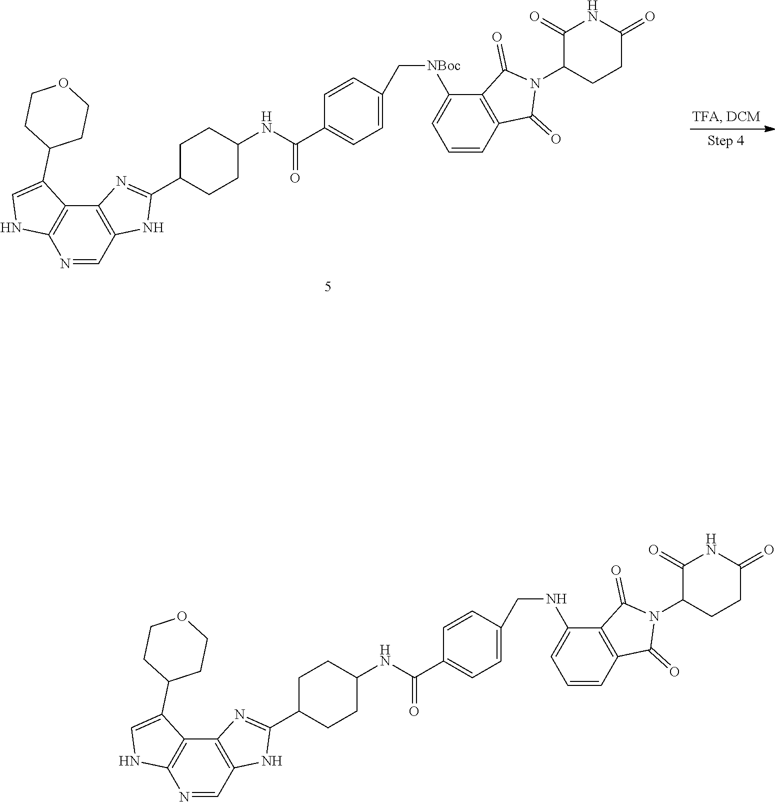 [SMILES: CC(C)(C)OC(=O)N(Cc1ccc(C(=O)NC2CCC(c3nc4c(cnc5[nH]cc(C6CCOCC6)c54)[nH]3)CC2)cc1)c1cccc2c1C(=O)N(C1CCC(=O)NC1=O)C2=O ; O=C1CCC(N2C(=O)c3cccc(NCc4ccc(C(=O)NC5CCC(c6nc7c(cnc8[nH]cc(C9CCOCC9)c87)[nH]6)CC5)cc4)c3C2=O)C(=O)N1]

In a 100 mL single-neck flask, 5 (82 mg, 0.099 mmol) was dissolved in DCM (2 mL), and TFA (0.4 mL) was added. The mixture was reacted at room temperature for 16 h and concentrated to dryness by rotary evaporation, and the residue was purified by preparative high performance liquid chromatography to give the target product (3.6 mg, 5.0% yield).

LC-MS: 729 [M+H]⁺, 1H NMR (400 MHz, DMSO) δ 12.15 (s, 1H), 11.13 (s, 1H), 8.74 (s, 1H), 8.33 (d, J=7.9 Hz, 1H), 7.83 (d, J=8.2 Hz, 2H), 7.51 (d, J=8.3 Hz, 1H), 7.48 (d, J=4.8 Hz, 1H), 7.45 (s, 2H), 7.33 (s, 1H), 7.04 (d, J=7.0 Hz, 1H), 6.92 (d, J=8.6 Hz, 1H), 5.08 (s, 1H), 4.63 (d, J=5.7 Hz, 2H), 4.02-3.88 (m, 5H), 3.63 (d, J=10.9 Hz, 2H), 3.18 (s, 1H), 2.94-2.87 (m, 1H), 2.22 (d, J=11.3 Hz, 2H), 2.07-1.92 (m, 6H), 1.85-1.71 (m, 4H), 1.58 (s, 2H).

Other representative example compounds are shown in the table below, but are not limited to these.

TABLE 1
Other representative example compounds

| Example | |
|---|---|
| No. | Structure |
| 117 | 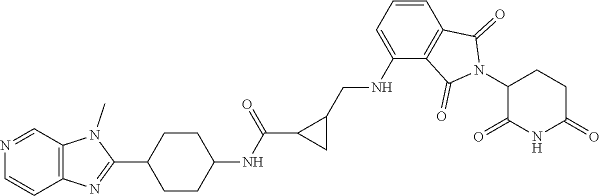 [SMILES: Cn1c(C2CCC(NC(=O)C3CC3CNc3cccc4c3C(=O)N(C3CCC(=O)NC3=O)C4=O)CC2)nc2ccncc21] |
| 118 | 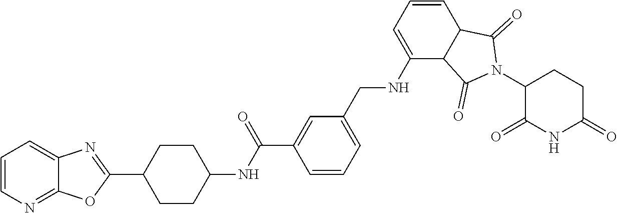 [SMILES: O=C1CCC(N2C(=O)C3C=CC=C(NCc4cccc(C(=O)NC5CCC(c6nc7cccnc7o6)CC5)c4)C3C2=O)C(=O)N1] |
| 119 | 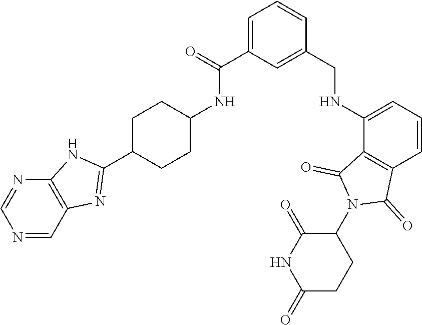 [SMILES: O=C1CCC(N2C(=O)c3cccc(NCc4cccc(C(=O)NC5CCC(c6nc7cncnc7[nH]6)CC5)c4)c3C2=O)C(=O)N1] |
| 120 | 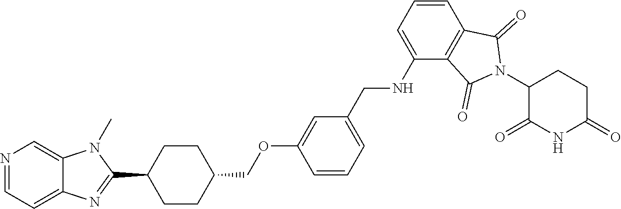 [SMILES: Cn1c2cnccc2nc1[C@H]1CC[C@H](COc2cccc(CNc3cccc4c3C(=O)N(C3CCC(=O)NC3=O)C4=O)c2)CC1] |
| 121 | 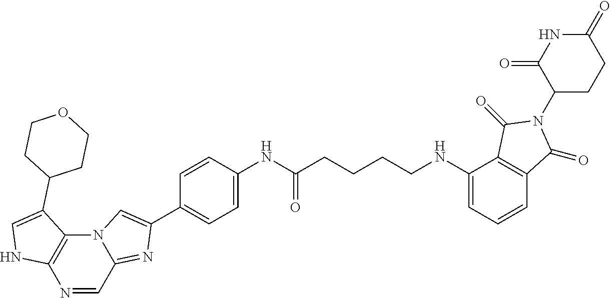 [SMILES: O=C1CCC(N2C(=O)c3cccc(NCCCCC(=O)Nc4ccc(-c5cn6c(cnc7[nH]cc(C8CCOCC8)c76)n5)cc4)c3C2=O)C(=O)N1] |
| 122 | 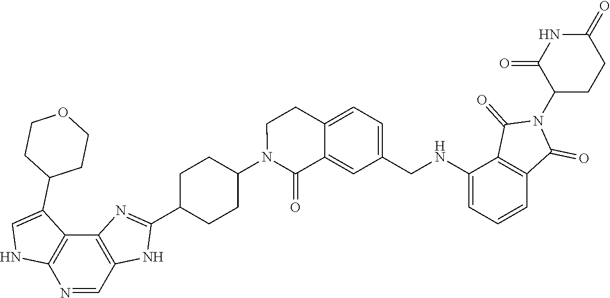 [SMILES: O=C1CCC(N2C(=O)c3cccc(NCc4ccc5c(c4)C(=O)N(C4CCC(c6nc7c(cnc8[nH]cc(C9CCOCC9)c87)[nH]6)CC4)CC5)c3C2=O)C(=O)N1] |
| 123 | 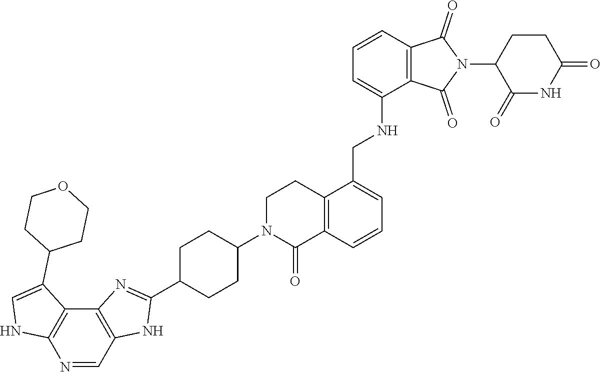 [SMILES: O=C1CCC(N2C(=O)c3cccc(NCc4cccc5c4CCN(C4CCC(c6nc7c(cnc8[nH]cc(C9CCOCC9)c87)[nH]6)CC4)C5=O)c3C2=O)C(=O)N1] |
| 124 | 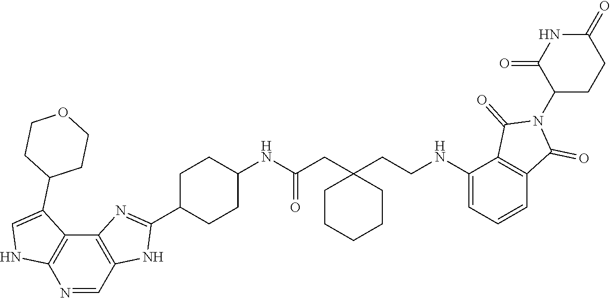 [SMILES: O=C1CCC(N2C(=O)c3cccc(NCCC4(CC(=O)NC5CCC(c6nc7c(cnc8[nH]cc(C9CCOCC9)c87)[nH]6)CC5)CCCCC4)c3C2=O)C(=O)N1] |
| 125 | 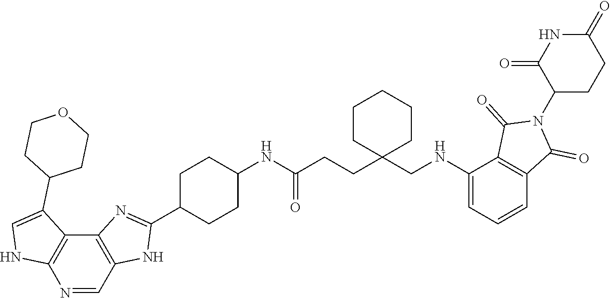 [SMILES: O=C1CCC(N2C(=O)c3cccc(NCC4(CCC(=O)NC5CCC(c6nc7c(cnc8[nH]cc(C9CCOCC9)c87)[nH]6)CC5)CCCCC4)c3C2=O)C(=O)N1] |
| 126 | 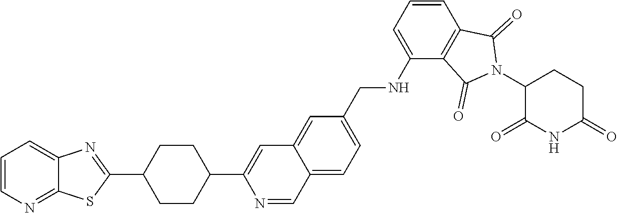 [SMILES: O=C1CCC(N2C(=O)c3cccc(NCc4ccc5cnc(C6CCC(c7nc8cccnc8s7)CC6)cc5c4)c3C2=O)C(=O)N1] |
| 127 | 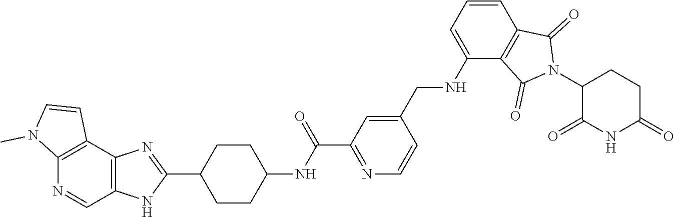 [SMILES: Cn1ccc2c3nc(C4CCC(NC(=O)c5cc(CNc6cccc7c6C(=O)N(C6CCC(=O)NC6=O)C7=O)ccn5)CC4)[nH]c3cnc21] |
| 128 | 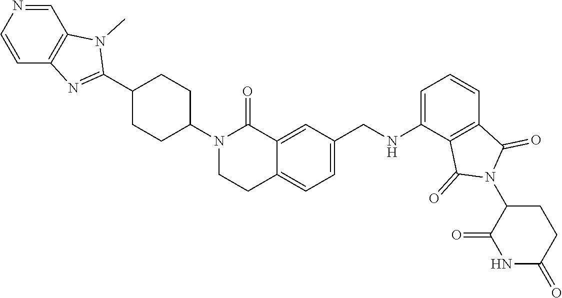 [SMILES: Cn1c(C2CCC(N3CCc4ccc(CNc5cccc6c5C(=O)N(C5CCC(=O)NC5=O)C6=O)cc4C3=O)CC2)nc2ccncc21] |
| 129 | 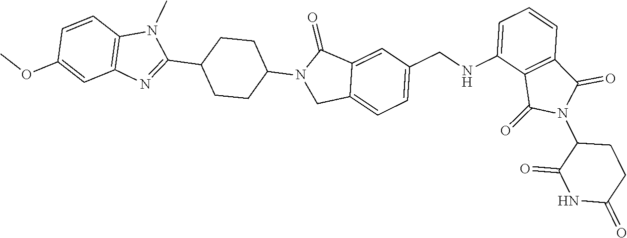 [SMILES: COc1ccc2c(c1)nc(C1CCC(N3Cc4ccc(CNc5cccc6c5C(=O)N(C5CCC(=O)NC5=O)C6=O)cc4C3=O)CC1)n2C] |

### Example 130

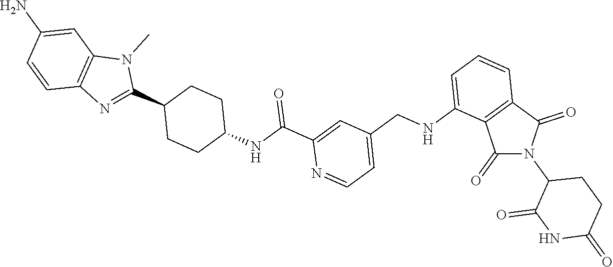 [SMILES: Cn1c2cc(N)ccc2nc1[C@H]1CC[C@H](NC(=O)c2cc(CNc3cccc4c3C(=O)N(C3CCC(=O)NC3=O)C4=O)ccn2)CC1]

### Preparation Steps

### Step 1

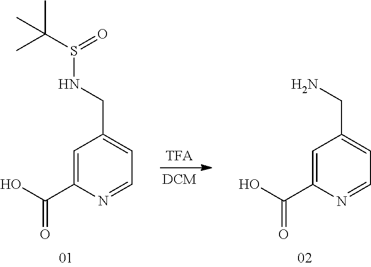 [SMILES: CC(C)(C)S(=O)NCc1ccnc(C(=O)O)c1 ; NCc1ccnc(C(=O)O)c1]

To a solution of compound 01 (83 g, 11.7 mmol) in DCM (30 mL) was added TFA (30 mL). The mixture was stirred at room temperature for 1 h. The reaction mixture was concentrated under reduced pressure to provide the corresponding compound 02 (1.6 g, Crude) as yellow solid. LC-MS: 153.0 [M+H]⁺

### Step 2

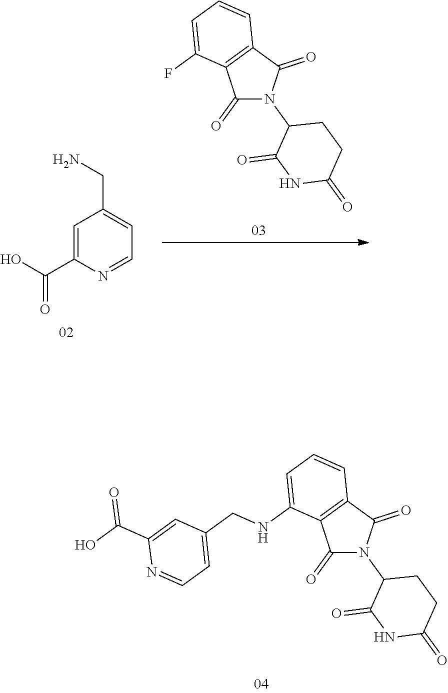 [SMILES: NCc1ccnc(C(=O)O)c1 ; O=C1CCC(N2C(=O)c3cccc(F)c3C2=O)C(=O)N1 ; O=C1CCC(N2C(=O)c3cccc(NCc4ccnc(C(=O)O)c4)c3C2=O)C(=O)N1]

The reaction mixture of compound 021.6 g, 1.05 mmol) and compound 03 (2.9 g, 1.05 mmol) in DMSO (10 mL) was added DIEA (4.1 g, 3.14 mmol) with stirring at 25° C. The mixture was stirred at 150° C. for 2 hours. The residue was purified by Prep-HPLC to afford compound 04 (600 mg, 13.99% yield) as yellow-green solid. LC-MS: 409.0 [M+H]⁺

### Step 3

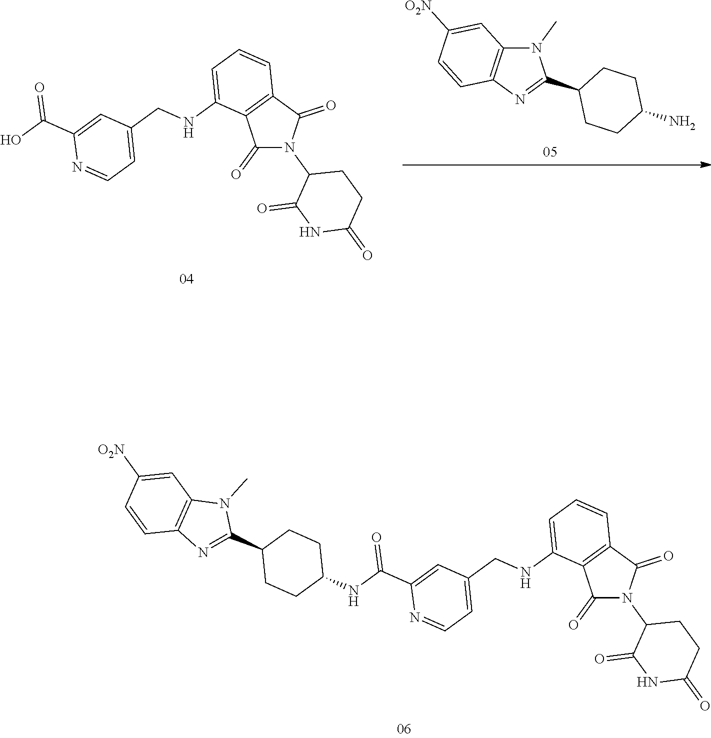 [SMILES: O=C1CCC(N2C(=O)c3cccc(NCc4ccnc(C(=O)O)c4)c3C2=O)C(=O)N1 ; Cn1c2cc([N+](=O)[O-])ccc2nc1[C@H]1CC[C@H](N)CC1 ; Cn1c2cc([N+](=O)[O-])ccc2nc1[C@H]1CC[C@H](NC(=O)c2cc(CNc3cccc4c3C(=O)N(C3CCC(=O)NC3=O)C4=O)ccn2)CC1]

To a mixture of compound 04 (600 mg, 1.47 mmol) in DMF (10 mL) were added compound 05 (403 mg, 1.47 mmol), NMI (603 mg, 7.35 mmol) and TCFH (494 mg, 1.76 mmol). The mixture stirred at room temperature for 1 h. The reaction mixture was purified by Prep-HPLC to afford the corresponding compound 06 (150 mg, 15.37%). LC-MS: 665.3 [M+H]⁺

### Step 4

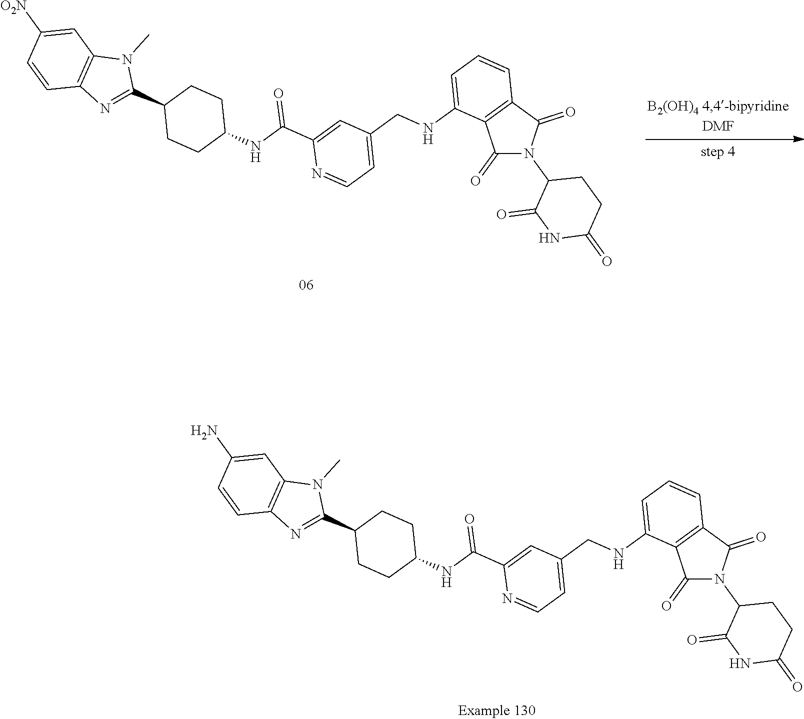 [SMILES: Cn1c2cc([N+](=O)[O-])ccc2nc1[C@H]1CC[C@H](NC(=O)c2cc(CNc3cccc4c3C(=O)N(C3CCC(=O)NC3=O)C4=O)ccn2)CC1 ; Cn1c2cc(N)ccc2nc1[C@H]1CC[C@H](NC(=O)c2cc(CNc3cccc4c3C(=O)N(C3CCC(=O)NC3=O)C4=O)ccn2)CC1]

To a mixture of compound 05 (150 mg, 0.23 mmol) in DMF (2 mL) were added 4,4-Bipyridine (50 mg, 0.34 mmol) and B₂(OH)₄ (60 mg, 0.78 mmol). The mixture was stirred at room temperature for 0.5 h. The reaction mixture was purified by Prep-HPLC to afford the corresponding Example 130 (37.2 mg, 26.0%). LC-MS: 635.3 [M+H]^{+ 1}H NMR (400 MHz, DMSO-d6) δ 11.12 (s, 1H), 8.57 (dd, J=14.4, 6.6 Hz, 2H), 8.04 (s, 1H), 7.57 (d, J=4.9 Hz, 1H), 7.54-7.45 (m, 2H), 7.19 (d, J=8.5 Hz, 1H), 7.05 (d, J=7.2 Hz, 1H), 6.87 (d, J=8.5 Hz, 1H), 6.52 (d, J=1.7 Hz, 2H), 6.47 (d, J=8.4 Hz, 1H), 5.10 (dd, J=12.8, 5.1 Hz, 1H), 4.71 (d, J=6.5 Hz, 2H), 3.90-3.80 (m, 1H), 3.59 (s, 3H), 2.90-2.81 (m, 2H), 2.65-2.58 (m, 3H), 2.03-1.92 (m, 5H), 1.70-1.50 (m, 4H).

### Example 131

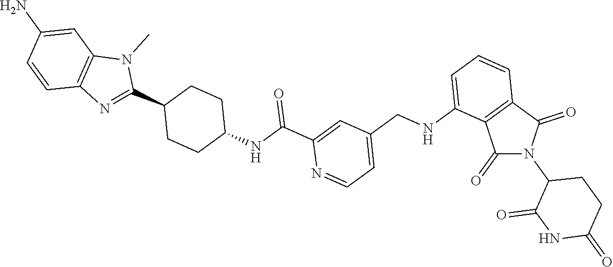 [SMILES: Cn1c2cc(N)ccc2nc1[C@H]1CC[C@H](NC(=O)c2cc(CNc3cccc4c3C(=O)N(C3CCC(=O)NC3=O)C4=O)ccn2)CC1]

### Step 1

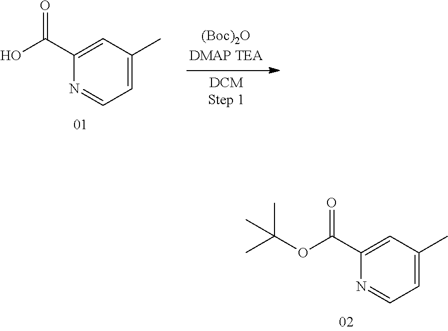

### Step 1

To a solution of compound 01 (5 g, 36.5 mmol) in DCM (50 mL) at 0° C. was added TEA (11 g, 10.9 mmol), DMAP (445 mg, 3.6 mmol) and (Boc)₂O (15.9 g, 7.3 mmol). The mixture was stirred at room temperature for 16 h. The reaction mixture was quenched with water and the aqueous layer was extracted with DCM (3×100 mL). The organic layer was concentrated and wash with brine (2×100 mL), dried over anhydrous Na₂SO₄, The organic layer was concentrated under reduced pressure. The obtained residue was purified by flash silica gel chromatography to provide the corresponding compound 02 (3.6 g, 51.43%), LC-MS: 194.0 [M+H]⁺

### Step 2

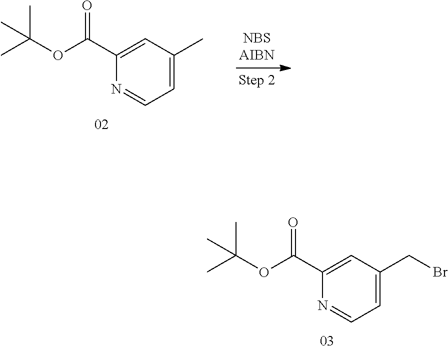

To a solution of compound 02 (3.6 g, 18.7 mmol) in CCl4 (40 mL) was added AIBN (306 mg, 1.87 mmol) and NBS (3.98 g, 22.4 mmol). The mixture was stirred at 80° C. for 4 h. The reaction mixture was quenched with water and the aqueous layer was extracted with DCM (3×50 mL). The organic layer was concentrated and wash with brine (2×30 mL), dried over anhydrous Na₂SO₄, The organic layer was concentrated under reduced pressure. The obtained residue was purified by flash silica gel chromatography to provide the corresponding compound 03 (1.8 g, 36.0%) as a yellow solid. LC-MS: 274.1 [M+3H]⁺

### Step 3

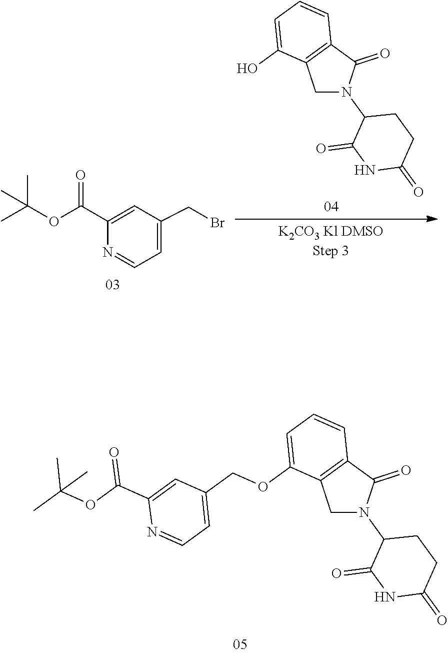 [SMILES: CC(C)(C)OC(=O)c1cc(CBr)ccn1 ; O=C1CCC(N2Cc3c(O)cccc3C2=O)C(=O)N1 ; CC(C)(C)OC(=O)c1cc(COc2cccc3c2CN(C2CCC(=O)NC2=O)C3=O)ccn1]

To a solution of compound 03 (1.8 g, 6.6 mmol) in DMSO (10 mL) was added compound 04 (1.73 g, 6.6 mmol), KI (551 mg, 3.32 mmol) and K₂CO₃ (1.1 g, 7.97 mmol). The mixture was stirred at room temperature for 3 h. The reaction mixture was quenched with water and the aqueous layer was extracted with EA (3×100 mL). The organic layer was concentrated and wash with brine (2×30 mL), dried over anhydrous Na₂SO₄, The organic layer was concentrated under reduced pressure. The obtained residue was purified by flash silica gel chromatography to provide the corresponding compound 05 (800 mg, 26.76%) as yellow solid. LC-MS: 452.1 [M+H]⁺

### Step 4

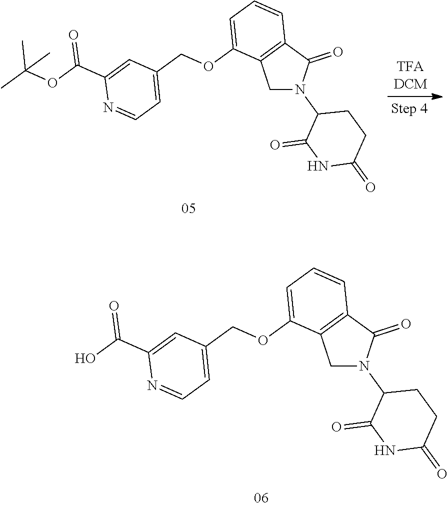 [SMILES: CC(C)(C)OC(=O)c1cc(COc2cccc3c2CN(C2CCC(=O)NC2=O)C3=O)ccn1 ; O=C1CCC(N2Cc3c(OCc4ccnc(C(=O)O)c4)cccc3C2=O)C(=O)N1]

To a solution of compound 05 (800 mg, 1.77 mmol) in DCM (10 mL) was added TFA (10 mL). The mixture was stirred at room temperature for 16 h. The reaction mixture was concentrated under reduced pressure to provide the corresponding compound 06 (600 mg, Crude) as a yellow solid. LC-MS: 396.0 [M+H]⁺

### Step 5

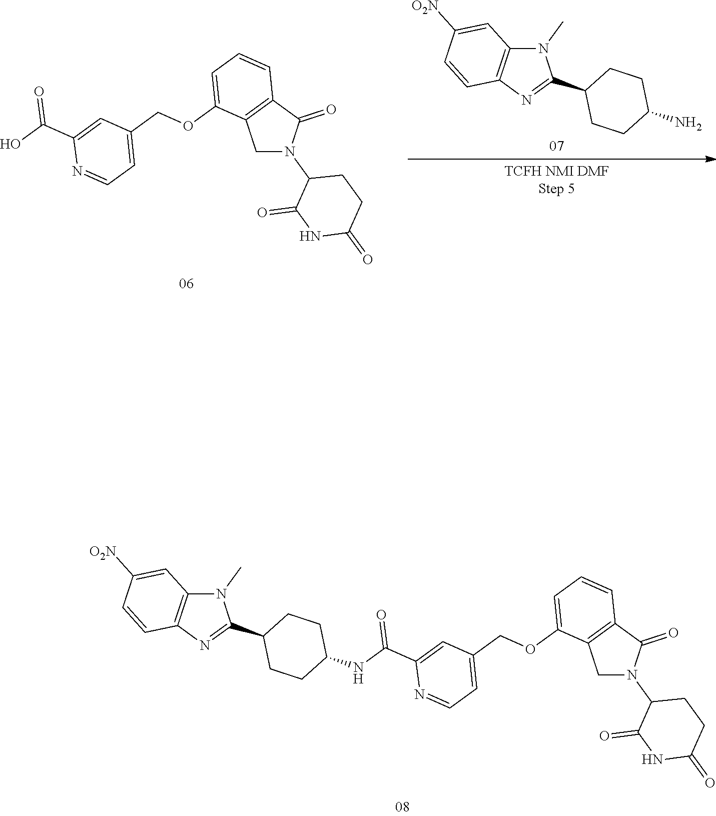 [SMILES: O=C1CCC(N2Cc3c(OCc4ccnc(C(=O)O)c4)cccc3C2=O)C(=O)N1 ; Cn1c2cc([N+](=O)[O-])ccc2nc1[C@H]1CC[C@H](N)CC1 ; Cn1c2cc([N+](=O)[O-])ccc2nc1[C@H]1CC[C@H](NC(=O)c2cc(COc3cccc4c3CN(C3CCC(=O)NC3=O)C4=O)ccn2)CC1]

To a mixture of compound 06 (600 mg, 1.52 mmol) in DMF (10 mL) were added compound 07 (416 mg, 1.52 mmol), NMI (623 mg, 7.6 mmol) and TCFH (510 mg, 1.8 mmol). The mixture stirred at room temperature for 16 h. The reaction mixture was purified by Prep-HPLC to afford the corresponding compound 08 (280 mg, 24.3%). LC-MS: 652.8 [M+H]⁺

### Step 6

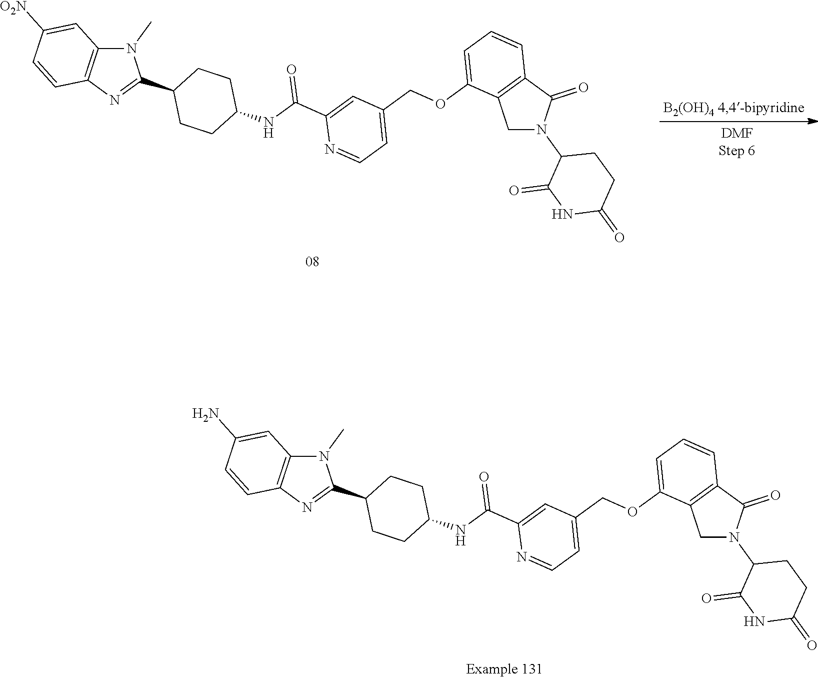 [SMILES: Cn1c2cc([N+](=O)[O-])ccc2nc1[C@H]1CC[C@H](NC(=O)c2cc(COc3cccc4c3CN(C3CCC(=O)NC3=O)C4=O)ccn2)CC1 ; Cn1c2cc(N)ccc2nc1[C@H]1CC[C@H](NC(=O)c2cc(COc3cccc4c3CN(C3CCC(=O)NC3=O)C4=O)ccn2)CC1]

To a mixture of compound 08 (150 mg, 0.23 mmol) in DMF (3 mL) were added 4,4-Bipyridine (54 mg, 0.35 mmol) and B₂(OH)₄ (62 mg, 0.7 mmol). The mixture was stirred at room temperature for 0.5 h. The reaction mixture was purified by Prep-HPLC to afford the corresponding Example 131 (87.33 mg, 55.48%) as a white solid. LC-MS: 622.3 [M+H]^{+ 1}H NMR (400 MHz, DMSO-d6) δ 10.99 (s, 1H), 8.67 (d, J=5.0 Hz, 1H), 8.59 (d, J=8.5 Hz, 1H), 8.13 (s, 1H), 7.71 (d, J=3.9 Hz, 1H), 7.50 (t, J=7.8 Hz, 1H), 7.36 (d, J=7.4 Hz, 1H), 7.29 (d, J=8.1 Hz, 1H), 7.20 (d, J=8.4 Hz, 1H), 6.52 (d, J=1.8 Hz, 1H), 6.47 (dd, J=8.4, 1.9 Hz, 1H), 5.46 (s, 2H), 5.14 (dd, J=13.2, 5.1 Hz, 1H), 4.43 (dd, J=62.2, 17.5 Hz, 2H), 3.94-3.82 (m, 1H), 3.60 (s, 3H), 3.00-2.77 (m, 2H), 2.64-2.56 (m, 2H), 2.48-2.40 (m, 2H), 2.06-1.94 (m, 5H), 1.85-1.50 (m, 4H).

### Example 132

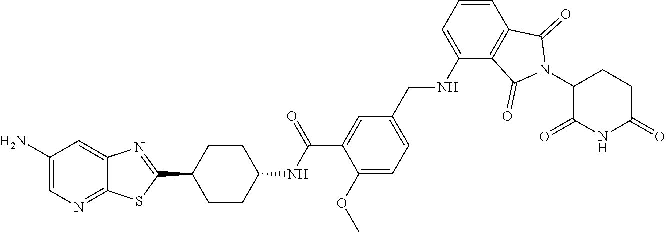 [SMILES: COc1ccc(CNc2cccc3c2C(=O)N(C2CCC(=O)NC2=O)C3=O)cc1C(=O)N[C@H]1CC[C@H](c2nc3cc(N)cnc3s2)CC1]

### Step 1

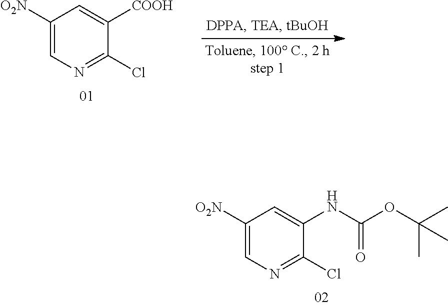 [SMILES: O=C(O)c1cc([N+](=O)[O-])cnc1Cl ; CC(C)(C)OC(=O)Nc1cc([N+](=O)[O-])cnc1Cl]

To the reaction mixture of compound 01 (25 g, 123.8 mmol) in Toluene (300 mL) was added DPPA (17 g, 61.9 mmol), TEA (37.5 g, 371.3 mmol) and t-BuOH (98 g, 185.6 mmol) with stirring at 25° C. The mixture was stirred at 100° C. for 2 hours. The reaction mixture was added water (300 mL) and extracted with EA (300 mL*3), washed with brine and dried concentrated to crude product which was further purified by com-flash (PE:EA=2:3) to afford compound 02 (15 g, 44.4%) as a yellow solid. LC-MS: (ESI, m/z): [M+H]⁺=274.0

### Step 2

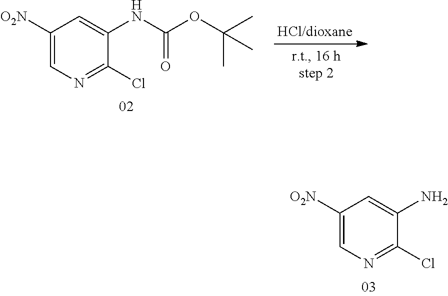 [SMILES: CC(C)(C)OC(=O)Nc1cc([N+](=O)[O-])cnc1Cl ; Nc1cc([N+](=O)[O-])cnc1Cl]

To a reaction mixture of compound 02 (14.5 g, 53.1 mmol) in DCM (150 mL) was added HCl/dioxane (150 mL) with stirring at 25° C. The mixture was stirred at room temperature for 16 hours. The reaction mixture was concentrated under reduced pressure to obtain the crude product. The crude product which was further purified by com-flash (DCM:MeOH=8:1) to afford compound 03 (8.7 g, 94.5%) as a yellow solid. LC-MS: (ESI, m/z): [M+H]⁺=174.0

### Step 3

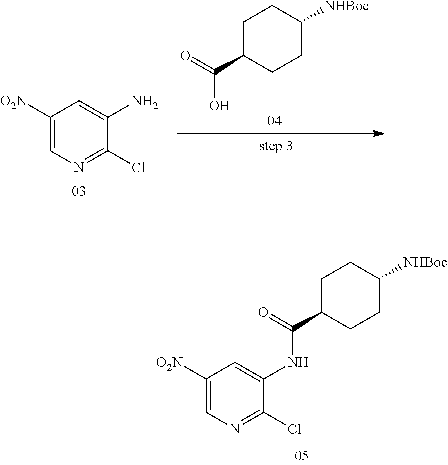 [SMILES: Nc1cc([N+](=O)[O-])cnc1Cl ; CC(C)(C)OC(=O)N[C@H]1CC[C@H](C(=O)O)CC1 ; CC(C)(C)OC(=O)N[C@H]1CC[C@H](C(=O)Nc2cc([N+](=O)[O-])cnc2Cl)CC1]

The reaction mixture of compound 03 (8.7 g, 50.3 mmol) in DMF (100 mL) was added compound 04 (12.2 g, 50.3 mmol) and NMI (20.6 g, 251.4 mmol) with stirring at room temperature. After 30 min at room temperature, TCFH (16.9 g, 60.3 mmol) was added to this reaction at room temperature. The mixture was stirred at room temperature for 16 hours. The reaction mixture was added water (500 mL), extracted with EA (500 mL*3), washed with brine and dried concentrated to crude product which was further purified by com-flash (DCM:MeOH=10:1) to afford compound 05 (7.24 g, 36.2%) as a yellow solid. LC-MS: (ESI, m/z): [M+H]⁺=399.0

### Step 4

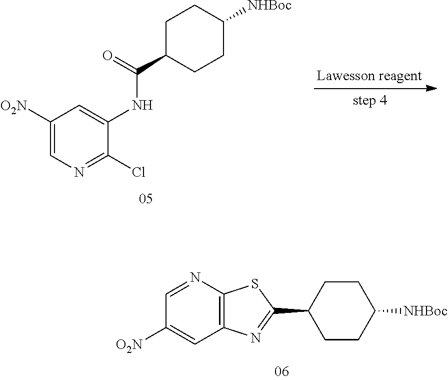 [SMILES: CC(C)(C)OC(=O)N[C@H]1CC[C@H](C(=O)Nc2cc([N+](=O)[O-])cnc2Cl)CC1 ; CC(C)(C)OC(=O)N[C@H]1CC[C@H](c2nc3cc([N+](=O)[O-])cnc3s2)CC1]

The reaction mixture of compound 05 (7.24 g, 18.2 mmol) in THF (100 mL) was added Lawesson reagent (11 g, 27.2 mmol) with stirring at room temperature. The mixture was stirred at 70° C. for 16 hours. The reaction mixture was concentrated, then NaHCO₃ (100 mL) was added, extracted with EA (100 mL*3), washed with brine and dried concentrated to crude product which was further purified by com-flash (DCM:MeOH=10:1) to afford compound 06 (8.2 g, crude) as a yellow solid. LC-MS: (ESI, m/z): [M+H]⁺=379.0

### Step 5

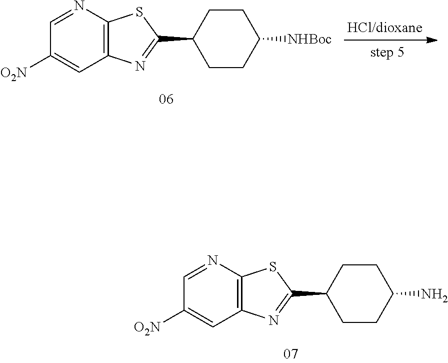 [SMILES: CC(C)(C)OC(=O)N[C@H]1CC[C@H](c2nc3cc([N+](=O)[O-])cnc3s2)CC1 ; N[C@H]1CC[C@H](c2nc3cc([N+](=O)[O-])cnc3s2)CC1]

The reaction mixture of compound 06 (8.2 g, 21.7 mmol) in DCM (100 mL) was added HCl/dioxane (100 mL) with stirring at room temperature. The mixture was stirred at room temperature for 16 hours. The reaction mixture was concentrated under reduced pressure to obtain the crude product. The crude product dissolved in ethyl acetate, then the mixture was filtered and the filtrate was concentrated under reduced pressure to obtain the product compound 07 (5.8 g, 96.7%) as a yellow solid. LC-MS: (ESI, m/z): [M+H]⁺=279.0

### Step 6

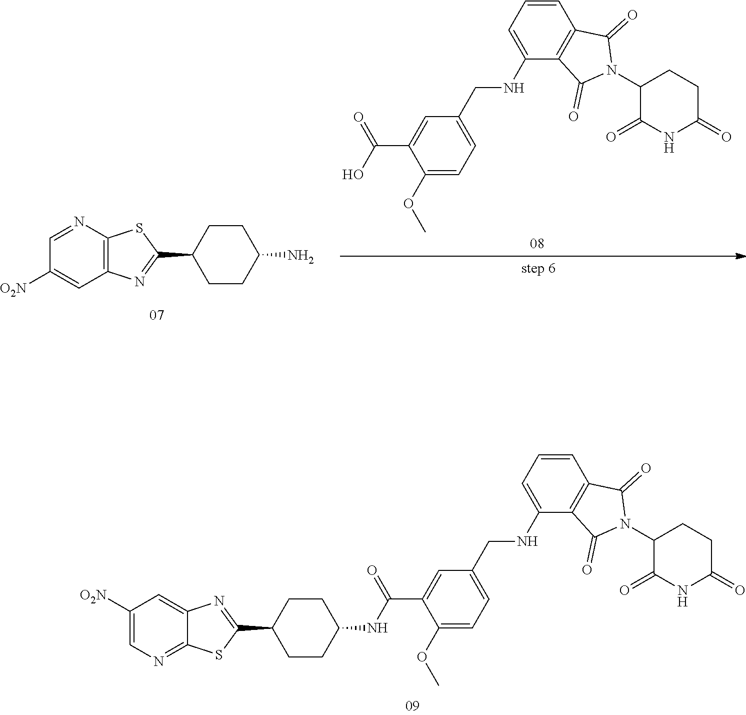 [SMILES: N[C@H]1CC[C@H](c2nc3cc([N+](=O)[O-])cnc3s2)CC1 ; COc1ccc(CNc2cccc3c2C(=O)N(C2CCC(=O)NC2=O)C3=O)cc1C(=O)O ; COc1ccc(CNc2cccc3c2C(=O)N(C2CCC(=O)NC2=O)C3=O)cc1C(=O)N[C@H]1CC[C@H](c2nc3cc([N+](=O)[O-])cnc3s2)CC1]

The reaction mixture of compound 07 (1 g, 2.3 mmol) in DMF (20 mL) was added compound 08 (636 mg, 2.3 mmol) and NMI (938 mg, 11.4 mmol) with stirring at room temperature. After 30 min at room temperature, TCFH (769 mg, 2.7 mmol) was added to this reaction at room temperature. The mixture was stirred at room temperature for 16 hours. The reaction mixture was added water, some solids are precipitated and filtered to obtain crude product. The crude product was purified by Prep-HPLC (water 2767/Qda, Column: Atlantis™ T3 Prep OBD™, 19*250 mm, 10 μm; Mobile Phase A: 0.1% FA/H2O, B: ACN; flow rate: 40 ml/min; gradient: 60%˜70%; Retention Time: 1.7-1.8 min of 3 min) to afford compound 09 (700 mg, 44.03% yield) as yellow-green solid. LC-MS: (ESI, m/z): [M+H]⁺=698.1.

### Step 7

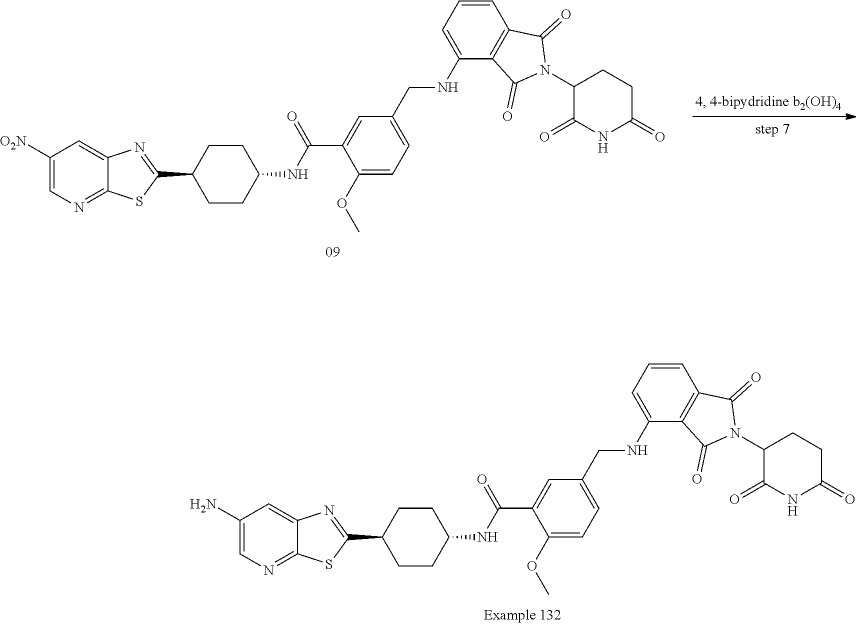 [SMILES: COc1ccc(CNc2cccc3c2C(=O)N(C2CCC(=O)NC2=O)C3=O)cc1C(=O)N[C@H]1CC[C@H](c2nc3cc([N+](=O)[O-])cnc3s2)CC1 ; COc1ccc(CNc2cccc3c2C(=O)N(C2CCC(=O)NC2=O)C3=O)cc1C(=O)N[C@H]1CC[C@H](c2nc3cc(N)cnc3s2)CC1]

The reaction mixture of compound 09 (700 mg, 1.0 mmol) in DMF (8 mL) was added 4,4′bipyridine (15 mg, 0.1 mmol) and B₂(OH)₄ (180 mg, 2.0 mmol) stirring at Rt. The reaction mixture was stirred at room temperature for 10 min. The residue was purified by Prep-HPLC to afford Compound Example 132 (108.23 mg). LC-MS: (ESI, m/z): [M+H]⁺=668.2. ¹H NMR (400 MHz, DMSO-d6) δ 11.09 (s, 1H), 8.02-7.97 (m, 2H), 7.73 (d, J=2.1 Hz, 1H), 7.54-7.45 (m, 2H), 7.37 (d, J=2.4 Hz, 1H), 7.23 (t, J=6.1 Hz, 1H), 7.11 (d, J=8.6 Hz, 1H), 7.00 (dd, J=15.3, 7.8 Hz, 2H), 5.48 (s, 2H), 5.07 (dd, J=13.0, 5.3 Hz, 1H), 4.52 (d, J=5.9 Hz, 2H), 3.88-3.78 (m, 4H), 3.08-2.93 (m, 1H), 2.90-2.83 (m, 1H), 2.61-2.54 (m, 2H), 2.20-2.15 (m, 2H), 2.07-1.99 (m, 3H), 1.73-1.64 (m, 2H), 1.53-1.43 (m, 2H).

### Example 133

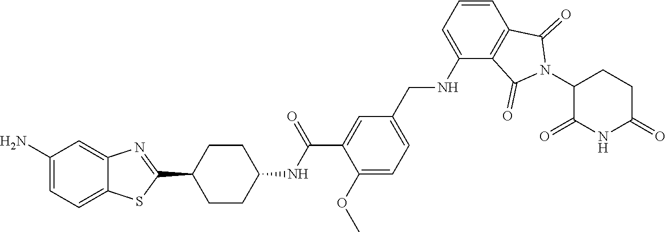 [SMILES: COc1ccc(CNc2cccc3c2C(=O)N(C2CCC(=O)NC2=O)C3=O)cc1C(=O)N[C@H]1CC[C@H](c2nc3cc(N)ccc3s2)CC1]

### Step 1

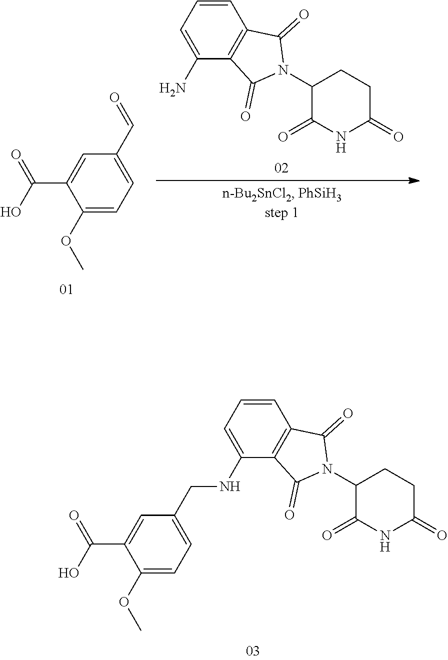 [SMILES: COc1ccc(C=O)cc1C(=O)O ; Nc1cccc2c1C(=O)N(C1CCC(=O)NC1=O)C2=O ; COc1ccc(CNc2cccc3c2C(=O)N(C2CCC(=O)NC2=O)C3=O)cc1C(=O)O]

The reaction mixture of compound 01 (1.45 g, 8.1 mmol) in THF (40 mL) was added compound 02 (2 g, 7.3 mmol), n-Bu₂SnCl₂ (2.45 g, 8.1 mmol) and PhSiH₃ (791 mg, 7.3 mmol) with stirring at 25° C. The mixture was stirred at 80° C. for 16 hours. The reaction mixture was concentrated under reduced pressure to obtain the crude product. The crude product which was further purified by com-flash (DCM:MeOH=10:1) to afford compound 03 (1.4 g, 37.5%) as a green solid. LC-MS: (ESI, m/z): [M+H]⁺=455.0

### Step 2

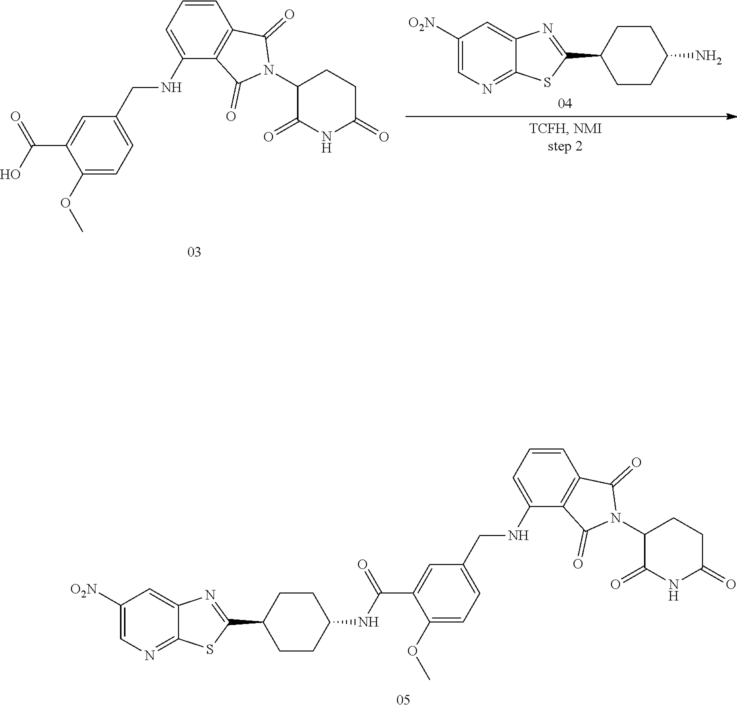 [SMILES: COc1ccc(CNc2cccc3c2C(=O)N(C2CCC(=O)NC2=O)C3=O)cc1C(=O)O ; N[C@H]1CC[C@H](c2nc3cc([N+](=O)[O-])cnc3s2)CC1 ; COc1ccc(CNc2cccc3c2C(=O)N(C2CCC(=O)NC2=O)C3=O)cc1C(=O)N[C@H]1CC[C@H](c2nc3cc([N+](=O)[O-])cnc3s2)CC1]

A reaction mixture of compound 03 (300 mg, 0.69 mmol) in DMF (2 mL) was added compound 04 (190 mg, 0.69 mmol) and NMI (281 mg, 3.43 mmol) with stirring at room temperature. After 10 min at room temperature, TCFH (230 mg, 0.84 mmol) was added to this reaction at room temperature. The mixture was stirred at room temperature for 1 hours. The mixture was purified by Prep-HPLC to afford compound 05 (120 mg, 25.1% yield) as a yellow-green solid. LC-MS: (ESI, m/z): [M+H]⁺=697.2

### Step 3

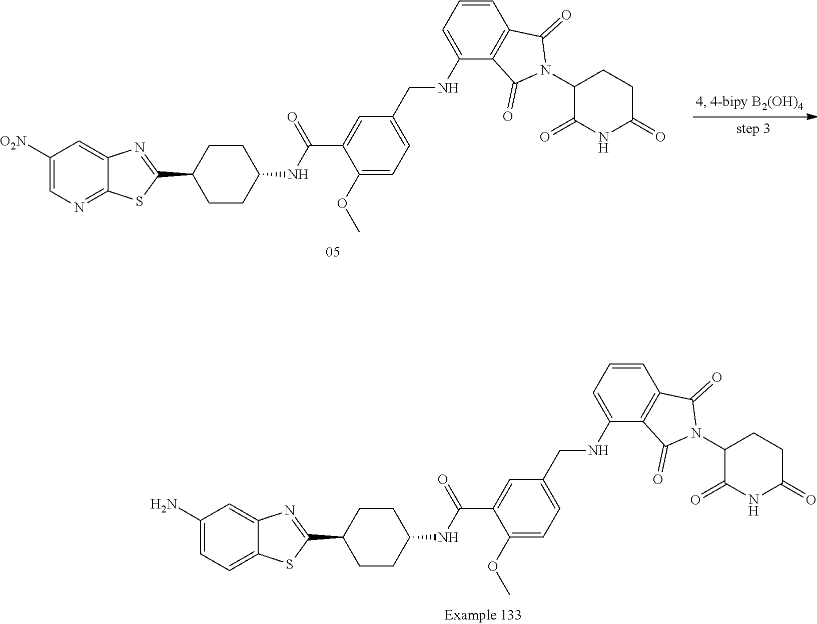 [SMILES: COc1ccc(CNc2cccc3c2C(=O)N(C2CCC(=O)NC2=O)C3=O)cc1C(=O)N[C@H]1CC[C@H](c2nc3cc([N+](=O)[O-])cnc3s2)CC1 ; COc1ccc(CNc2cccc3c2C(=O)N(C2CCC(=O)NC2=O)C3=O)cc1C(=O)N[C@H]1CC[C@H](c2nc3cc(N)ccc3s2)CC1]

The reaction mixture of compound 09 (120 mg, 0.172 mmol) in DMF (2 mL) was added 4,4′bipyridine (2.7 mg, 0.017 mmol) and B₂(OH)₄ (30 mg, 0.34 mmol) stirring at Rt. The reaction mixture was stirred at room temperature for 5 min. The residue was purified by Prep-HPLC to afford Compound Example 133 (46.84 mg, 40.8%) as yellow-green solid. LC-MS: (ESI, m/z): [M+H]⁺=667.4. ¹H NMR (400 MHz, DMSO-d6) δ 11.09 (s, 1H), 8.00 (d, J=7.7 Hz, 1H), 7.73 (s, 1H), 7.60 (d, J=8.5 Hz, 1H), 7.50 (dd, J=15.2, 7.5 Hz, 2H), 7.24 (s, 1H), 7.16-6.90 (m, 4H), 6.71 (d, J=8.5 Hz, 1H), 5.21 (s, 2H), 5.07 (dd, J=12.7, 5.2 Hz, 1H), 4.52 (d, J=5.8 Hz, 211), 3.89-3.74 (m, 411), 3.04-2.84 (m, 2H), 2.66-2.58 (m, 1H), 2.20-2.16 (m, 2H), 2.10-2.02 (m, 4H), 1.78-1.62 (m, 2H), 1.59-1.38 (m, 2H).

### Example 134

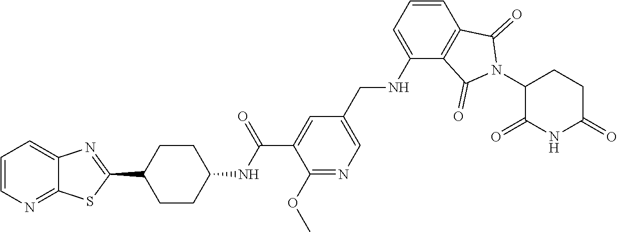

### Step 1

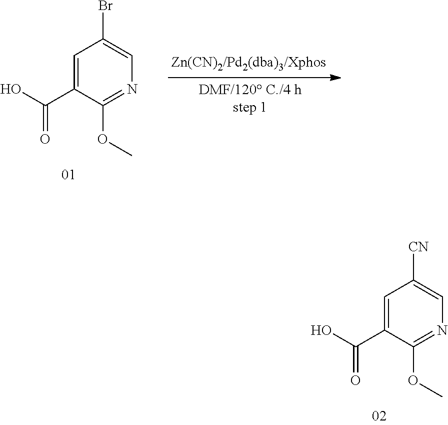

The reaction mixture of compound 01 (2 g, 8.62 mmol) in DMF (30 mL) was added X-Phos (410 mg, 0.86 mmol), Pd₂(dba)₃ (402 mg, 0.43 mmol) and ZnCN (5 g, 43.1 mmol) with stirring at 25° C. The mixture was stirred at 120° C. for 4 hours. The reaction mixture was added water (300 mL) and extracted with EA (300 mL*3), washed with brine and dried concentrated to crude product which was further purified by com-flash (DCM:MeOH=5:1) to afford compound 02 (600 mg, 38.96%) as a white solid. LC-MS: (ESI, m/z): [M+H]⁺=177.2

### Step 2

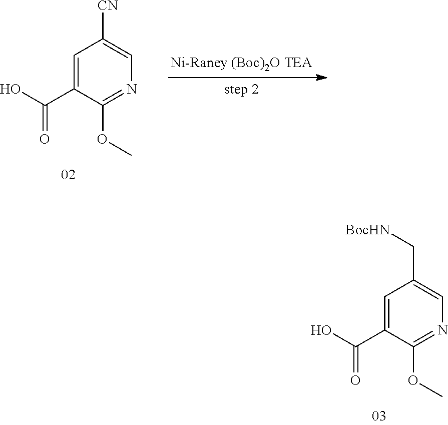 [SMILES: COc1ncc(C#N)cc1C(=O)O ; COc1ncc(CNC(=O)OC(C)(C)C)cc1C(=O)O]

A reaction mixture of compound 02 (600 mg, 3.37 mmol) in MeOH (10 mL) was added TEA (1.7 g, 16.85 mmol), (Boc)₂O (2.2 g, 10.11 mmol) and Ni-Raney (1 mL) with stirring at 25° C. The mixture was stirred in an autoclave with hydrogen at room temperature at 3 MPa for 16 h. The reaction mixture was filtered and the filtrate was concentrated under reduced pressure to obtain the crude product. The crude product which was further purified by com-flash (DCM:MeOH=8:1) to afford compound 03 (300 mg, 31.58%) as a white solid. LC-MS: (ESI, m/z): [M+H]⁺=283.1

### Step 3

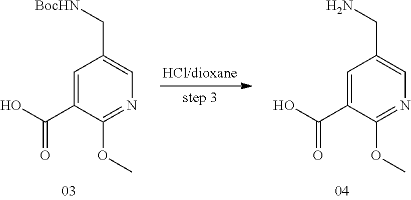 [SMILES: COc1ncc(CNC(=O)OC(C)(C)C)cc1C(=O)O ; COc1ncc(CN)cc1C(=O)O]

The reaction mixture of compound 03 (300 mg, 1.06 mmol) in DCM (50 mL) was added HCl/dioxane (50 mL) with stirring at 25° C. The mixture was stirred at room temperature for 1 hours. The reaction mixture was concentrated under reduced pressure to obtain the crude product.compound 04 (210 mg, crude) as a white solid. LC-MS: (ESI, m/z): [M−NH₃]⁺=166.1

### Step 4

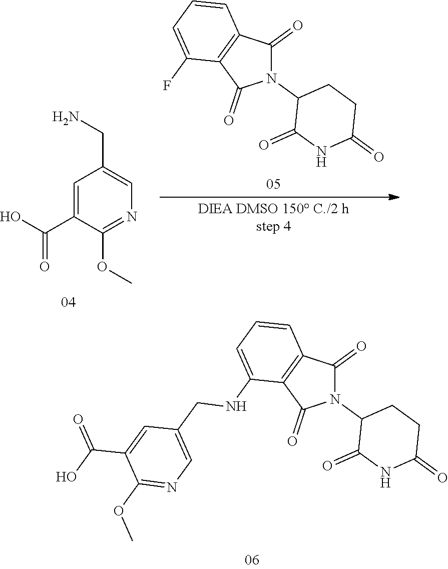 [SMILES: COc1ncc(CN)cc1C(=O)O ; O=C1CCC(N2C(=O)c3cccc(F)c3C2=O)C(=O)N1 ; COc1ncc(CNc2cccc3c2C(=O)N(C2CCC(=O)NC2=O)C3=O)cc1C(=O)O]

The reaction mixture of compound 04 (210 mg, 1.15 mmol) and compound 05 (318 mg, 1.15 mmol) in DMSO (2 mL) was added DIEA (1.04 g, 8.0 mmol) with stirring at 25° C. The mixture was stirred at 150° C. for 2 hours. The residue was purified by Prep-HPLC to afford compound 06 (60 mg, 11.88% yield) as yellow-green solid. LC-MS: (ESI, m/z): [M+Na]⁺=461.2

### Step 5

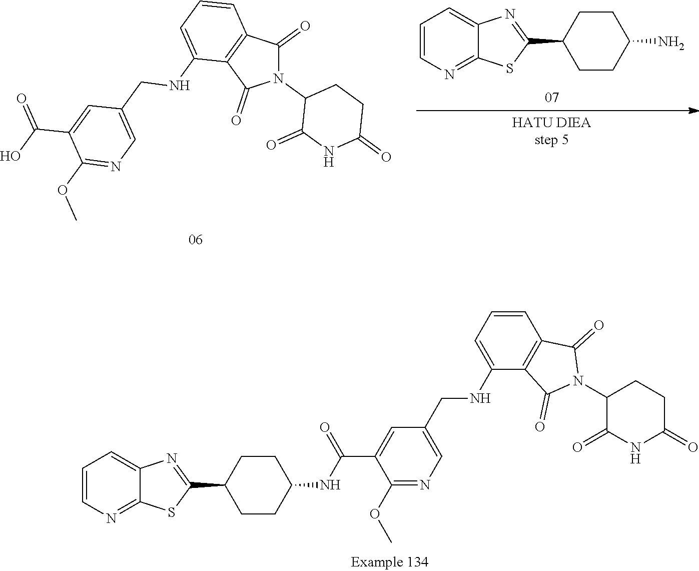 [SMILES: COc1ncc(CNc2cccc3c2C(=O)N(C2CCC(=O)NC2=O)C3=O)cc1C(=O)O ; N[C@H]1CC[C@H](c2nc3cccnc3s2)CC1 ; COc1ncc(CNc2cccc3c2C(=O)N(C2CCC(=O)NC2=O)C3=O)cc1C(=O)N[C@H]1CC[C@H](c2nc3cccnc3s2)CC1]

The reaction mixture of compound 06 (60 mg, 0.137 mmol) in DMF (1 mL) was added DIEA (88 mg, 0.68 mmol) and HATU (62 mg, 0.16 mmol) with stirring at 25° C. After 30 min at 25° C., compound 07 (32 mg, 0.137 mmol) was added to the reaction at 25° C. The mixture was stirred at 25° C. for 1 hours. The residue was purified by Prep-HPLC to afford Compound Example 134 (23.47 mg).

LC-MS: (ESI, m/z): [M+H]⁺=654.0; ¹H NMR (400 MHz, DMSO) δ 11.10 (s, 1H), 8.58 (d, J=4.6 Hz, 1H), 8.39-8.29 (m, 2H), 8.14 (s, 1H), 8.09 (d, J=7.4 Hz, 1H), 7.59-7.50 (m, 2H), 7.33 (s, 1H), 7.06 (dd, J=17.2, 7.7 Hz, 2H), 5.07 (dd, J=13.1, 4.9 Hz, 1H), 4.56 (d, J=6.0 Hz, 2H), 3.96 (s, 3H), 3.90-3.80 (m, 1H), 3.19-3.10 (m, 1H), 2.95-2.85 (m, 1H), 2.61-2.52 (m, 2H), 2.25-2.20 (m, 2H), 2.08-1.99 (m, 3H), 1.80-1.70 (m, 2H), 1.61-1.46 (m, 2H).

### Example 135

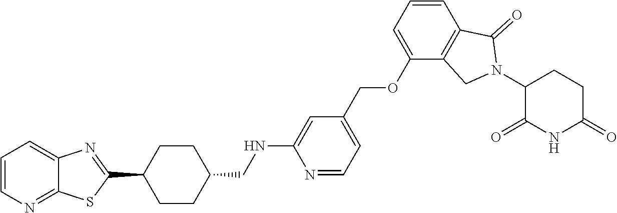 [SMILES: O=C1CCC(N2Cc3c(OCc4ccnc(NC[C@H]5CC[C@H](c6nc7cccnc7s6)CC5)c4)cccc3C2=O)C(=O)N1]

### Step 1

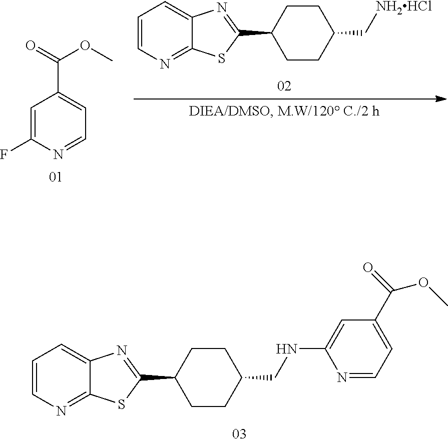

To a solution of compound 01 (1.84 g, 11.87 mmol) in 20 mL DMSO was added compound 02 (2.95 g, 11.94 mmol), DIEA (20.7 mL, 119.07 mmol), and the solution was stirred for 2 h at 120° C. under microwave. The reaction was diluted with water (160 mL) and extracted with EA (60 mL*3). The combined organic phases were washed with water (30 mL*2) and brine (30 mL), dried over anhydrous Na2SO4, filtered and concentrated to dryness in vacuo to give a yellow oil. The yellow oil was purified by com-flash (EA in PE=0-40%), the desired fractions were collected and concentrated to dryness in vacuo to give the desired product 03 as a yellow solid (232 mg, 9.4% yield). LC-MS: 383.2 [M+H]⁺

### Step 2

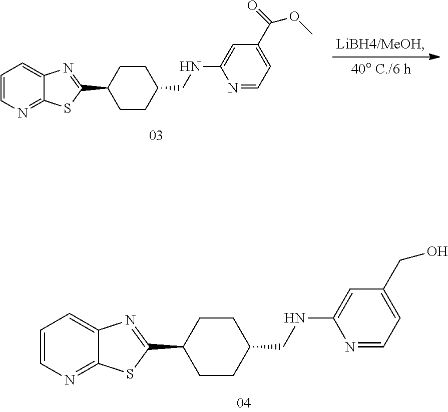 [SMILES: COC(=O)c1ccnc(NC[C@H]2CC[C@H](c3nc4cccnc4s3)CC2)c1 ; OCc1ccnc(NC[C@H]2CC[C@H](c3nc4cccnc4s3)CC2)c1]

To a solution of 03 (232 mg, 0.607 mmol) in MeOH (5 mL) was added LiBH4 (130 mg, 5.969 mmol) at 0° C., the reaction was removed from the ice base and returned to r.t. gradually, then the reaction was stirred at 40° C. for 6 h. Then 1 mL water was added dropwise at 0° C., the mixture was concentrated to dryness in vacuo to give a yellow oil. The yellow oil was purified by com-flash (EA in PE=0-100%), the desired fractions were collected and concentrated to dryness in vacuo to give the desired product 04 as a yellow oil (104 mg, 48.4% yield). LC-MS: 355.3 [M+H]⁺, TLC:PE:EA=0:1, Rf=0.4

### Step 3

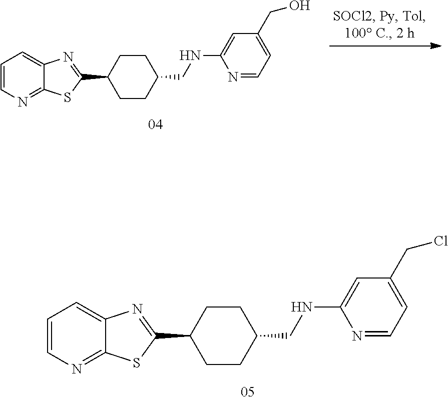 [SMILES: OCc1ccnc(NC[C@H]2CC[C@H](c3nc4cccnc4s3)CC2)c1 ; ClCc1ccnc(NC[C@H]2CC[C@H](c3nc4cccnc4s3)CC2)c1]

SOCl2 (42.7 uL, 0.588 mmol) was added into 04 (104 mg, 0.294 mmol) and Py (9.5 uL, 0.118 mmol) in Toluene (3 mL) at r.t., and it was heated at 100° C. for 2 h. The reaction was concentrated to dryness in vacuo to give a yellow oil. The yellow oil was purified by com-flash (MeOH in DCM=0-20%), the desired fractions were collected and concentrated to dryness in vacuo to give the desired product 05 as a yellow oil. (66 mg, 60.6% yield). LC-MS: 373.0[M+H]⁺, TLC:PE:EA=2:1, Rf=0.4

### Step 4

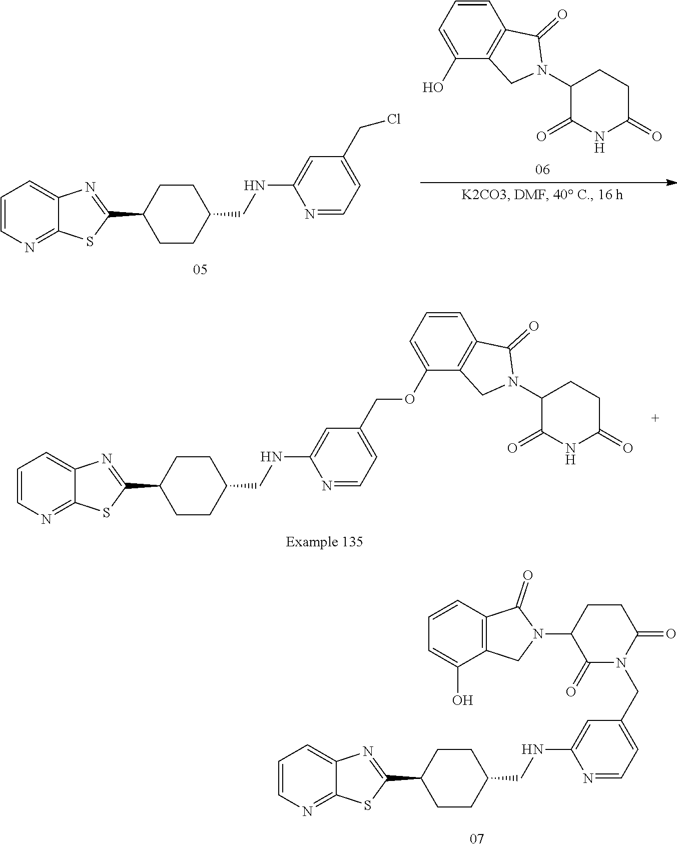 [SMILES: ClCc1ccnc(NC[C@H]2CC[C@H](c3nc4cccnc4s3)CC2)c1 ; O=C1CCC(N2Cc3c(O)cccc3C2=O)C(=O)N1 ; O=C1CCC(N2Cc3c(OCc4ccnc(NC[C@H]5CC[C@H](c6nc7cccnc7s6)CC5)c4)cccc3C2=O)C(=O)N1 ; O=C1CCC(N2Cc3c(O)cccc3C2=O)C(=O)N1Cc1ccnc(NC[C@H]2CC[C@H](c3nc4cccnc4s3)CC2)c1]

To a solution of 05 (60.0 mg, 0.161 mmol) in 1 mL DMF was added 06 (63 mg, 0.242 mmol), K2CO3 (134 mg, 0.970 mmol), the solution was stirred at 40° C. for 16 h. The reaction was purified by com-flash (ACN in NH4HCO3 (0.9% in water)=0-42%), the desired fractions were collected and lyophilized to give a yellow solid. The yellow solid was purified by Prep-HPLC to afford the target product Example 135 (2.81 mg) and compound 07.

Example 135: LC-MS: 597.4[M+H]⁺, ¹H NMR (400 MHz, DMSO-d6) (10.98 (s, 1H), 8.56 (d, J=3.8 Hz, 1H), 8.32 (d, J=8.7 Hz, 1H), 7.95 (d, J=5.0 Hz, 1H), 7.55 (dd, J=8.2, 4.6 Hz, 1H), 7.48 (t, J=7.6 Hz, 1H), 7.34 (d, J=7.5 Hz, 1H), 7.24 (d, J=8.0 Hz, 1H), 6.65 (s, 1H), 6.52 (d, J=6.4 Hz, 2H), 5.15 (d, J=6.5 Hz, 2H), 4.46 (d, J=17.5 Hz, 1H), 4.48-4.28 (m, 2H), 3.16-3.10 (m, 3H), 2.96-2.85 (m, 1H), 2.63-2.53 (m, 1H), 2.22-2.15 (m, 2H), 2.05-1.90 (m, 3H), 1.67-1.50 (m, 3H), 1.21-1.11 (m, 3H).

Compound 07: LC-MS: 597.4[M+H]⁺, ¹H NMR (400 MHz, DMSO-d6) δ 10.12 (s, 1H), 8.56 (d, J=3.2 Hz, 1H), 8.32 (d, J=8.2 Hz, 1H), 7.86 (d, J=5.3 Hz, 1H), 7.55 (dd, J=8.2, 4.7 Hz, 1H), 7.33 (t, J=7.7 Hz, 1H), 7.19 (d, J=7.4 Hz, 1H), 7.02 (d, J=8.0 Hz, 1H), 6.52 (s, 1H), 6.41-6.21 (m, 2H), 5.28 (dd, J=13.4, 5.0 Hz, 1H), 4.68 (s, 2H), 4.27 (dd, J=83.7, 17.1 Hz, 2H), 3.22-3.03 (m, 5H), 2.88-2.80 (m, 1H), 2.26-2.05 (m, 4H), 1.98-1.90 (m, 2H), 1.78-1.49 (m, 3H), 1.49-1.36 (m, 1H), 1.25-1.10 (m, 2H).

### Example 136

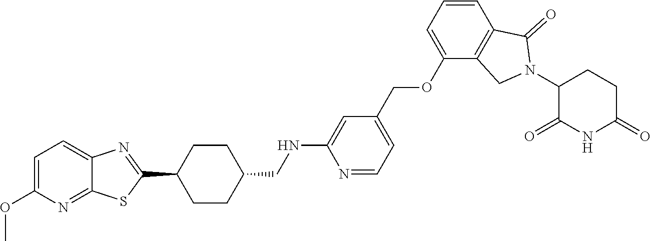 [SMILES: COc1ccc2nc([C@H]3CC[C@H](CNc4cc(COc5cccc6c5CN(C5CCC(=O)NC5=O)C6=O)ccn4)CC3)sc2n1]

### Step 1

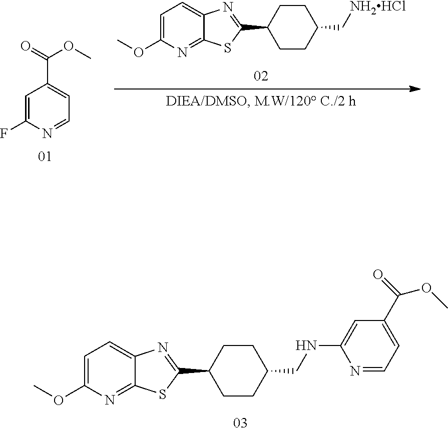

The reaction mixture of compound 01 (1.5 g, 9.68 mmol) and compound 02 (3.03 g, 9.67 mmol) in DMSO (10 mL) was added DIEA (16.8 mL, 96.6 mmol). The mixture was stirred at 150° C. for 2 hours. The reaction mixture was quenched with water and the aqueous layer was extracted with DCM (3×50 mL). The organic layer was concentrated and wash with brine (2×30 mL), dried over anhydrous Na₂SO₄, The organic layer was concentrated under reduced pressure. The obtained residue was purified by flash silica gel chromatography to provide the corresponding compound 03 (260 mg, 6.5%). LC-MS: 413.2 [M+H]⁺,

### Step 2

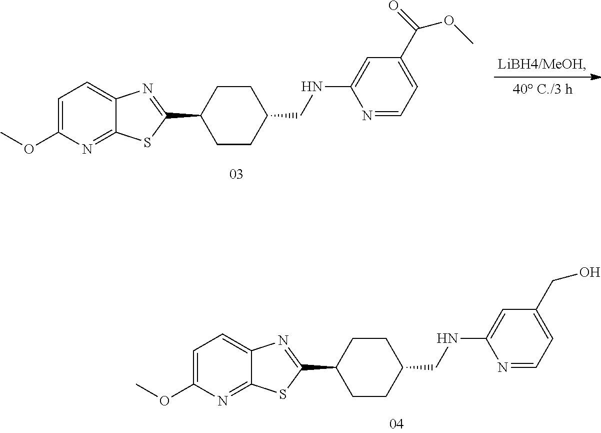

To a solution of compound 03 (260 mg, 0.631 mmol) in MeOH(3 mL) was added LiBH4 (41 mg, 1.882 mmol) at 0° C., the reaction was removed from the ice base and returned to r.t. gradually, then the reaction was stirred at 40° C. for 6 h. Then 1 mL water was added dropwise at 0° C., the mixture was concentrated to dryness in vacuo to give a yellow oil. The yellow oil was purified by com-flash (EA in PE=0-100%), the desired fractions were collected and concentrated to dryness in vacuo to give the desired product 04 as a yellow oil (220 mg, 90.9% yield). LC-MS: 345.2 [M+H]⁺

### Step 3

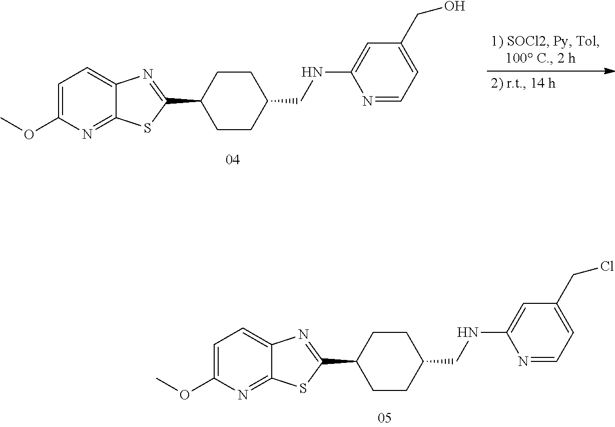 [SMILES: COc1ccc2nc([C@H]3CC[C@H](CNc4cc(CO)ccn4)CC3)sc2n1 ; COc1ccc2nc([C@H]3CC[C@H](CNc4cc(CCl)ccn4)CC3)sc2n1]

SOCl2 (83 uL, 1.143 mmol) was added into a solution of compound 04 (220 mg, 0.573 mmol) and Py (18 uL, 0.224 mmol) in Toluene (3 mL) at r.t., and it was heated at 100° C. for 2 h. The reaction was concentrated to dryness in vacuo to give a yellow oil. The yellow oil was purified by com-flash (MeOH in DCM=0-20%), the desired fractions were collected and concentrated to dryness in vacuo to give the desired product 05 as a yellow oil. (173 mg, 75.2% yield). LC-MS: 403.2[M+H]⁺

### Step 4

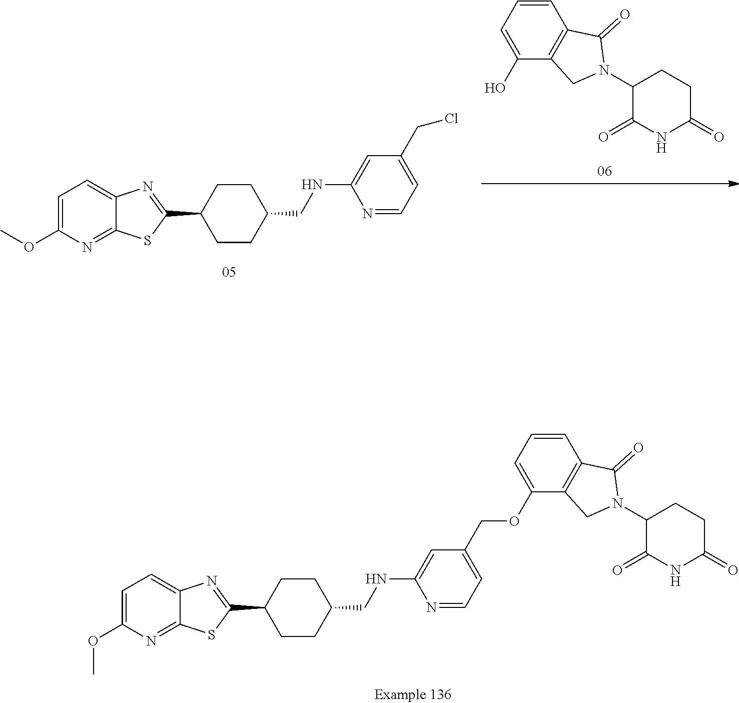 [SMILES: COc1ccc2nc([C@H]3CC[C@H](CNc4cc(CCl)ccn4)CC3)sc2n1 ; O=C1CCC(N2Cc3c(O)cccc3C2=O)C(=O)N1 ; COc1ccc2nc([C@H]3CC[C@H](CNc4cc(COc5cccc6c5CN(C5CCC(=O)NC5=O)C6=O)ccn4)CC3)sc2n1]

To a solution of compound 05 (55 mg, 0.14 mmol) in 1 mL of DMF was added compound 06 (71 mg, 0.27 mmol) and K2CO3 (77 mg, 0.56 mmol). The solution was stirred at 40° C. for 16 h. The reaction was purified by Prep-HPLC to afford the target product Example 136 (2.16 mg). LC-MS: 627.4 [M+H]⁺, ¹H NMR (400 MHz, DMSO-d6) δ 10.99 (s, 1H), 8.20 (d, J=8.9 Hz, 1H), 7.94 (d, J=5.3 Hz, 1H), 7.49 (t, J=7.7 Hz, 1H), 7.34 (d, J=7.5 Hz, 1H), 7.24 (d, J=8.1 Hz, 1H), 6.95 (d, J=8.8 Hz, 1H), 6.64 (s, 1H), 6.53 (t, J=4.5 Hz, 2H), 5.23-5.07 (m, 3H), 4.39 (dd, J=61.8, 17.4 Hz, 2H), 3.92 (s, 3H), 3.20-3.10 (m, 1H), 3.04 (m, 1H), 2.95-2.88 (m, 1H), 2.73-2.57 (m, 2H), 2.47-2.41 (m, 1H), 2.20-2.10 (m, 2H), 2.05-1.95 (m, 1H), 1.95-1.85 (m, 2H), 1.61-1.45 (m, 3H), 1.13 (m, 2H).

### Example 137

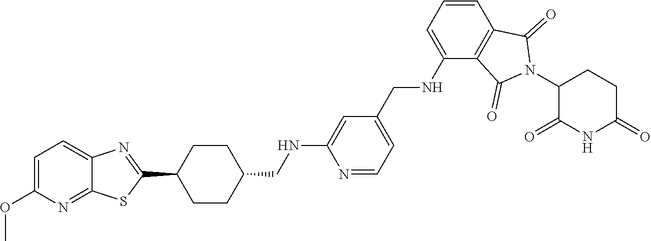 [SMILES: COc1ccc2nc([C@H]3CC[C@H](CNc4cc(CNc5cccc6c5C(=O)N(C5CCC(=O)NC5=O)C6=O)ccn4)CC3)sc2n1]

### Step 1

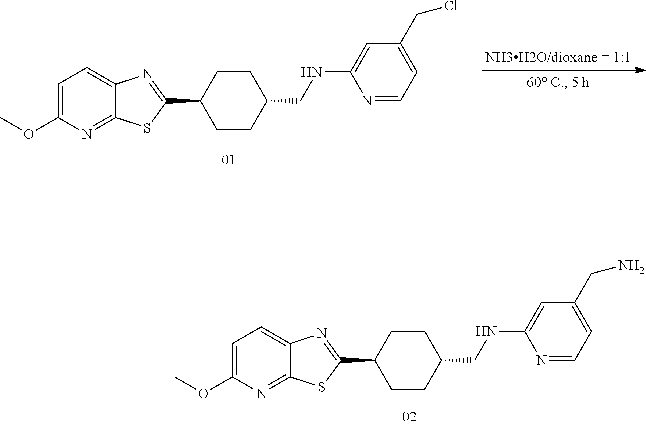 [SMILES: COc1ccc2nc([C@H]3CC[C@H](CNc4cc(CCl)ccn4)CC3)sc2n1 ; COc1ccc2nc([C@H]3CC[C@H](CNc4cc(CN)ccn4)CC3)sc2n1]

To a solution of compound 01 (60 mg, 0.15 mmol) in 3 mL NH3·H₂O/dioxane=1:1 was heated at 60° C. for 5 h. The reaction was concentrated to dryness in vacuo to give a yellow oil. The yellow oil was purified by com-flash (MeOH in DCM=0-37%), the desired fractions were collected and concentrated to dryness in vacuo to give the desired product 02 as a yellow solid. (55 mg, 96.3% yield). LC-MS: 384.1[M+H]⁺

### Step 2

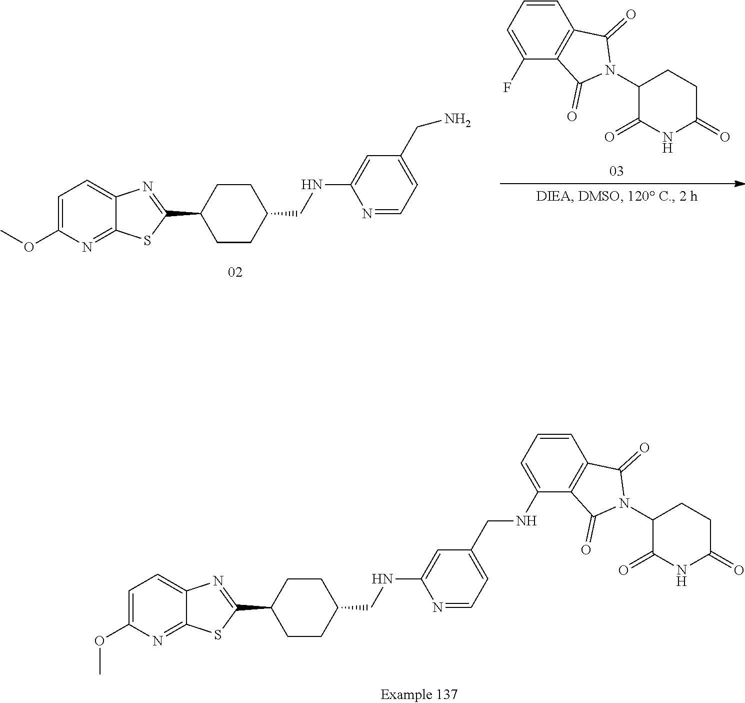 [SMILES: COc1ccc2nc([C@H]3CC[C@H](CNc4cc(CN)ccn4)CC3)sc2n1 ; O=C1CCC(N2C(=O)c3cccc(F)c3C2=O)C(=O)N1 ; COc1ccc2nc([C@H]3CC[C@H](CNc4cc(CNc5cccc6c5C(=O)N(C5CCC(=O)NC5=O)C6=O)ccn4)CC3)sc2n1]

To a solution of compound 02 (55 mg, 0.14 mmol) in 2 mL DMSO was added compound 03 (44 mg, 0.16 mmol), DIEA (0.12 mL, 0.690 mmol), the solution was stirred for 2 h at 120° C. under microwave. The reaction was purified by Prep-HPLC to afford the target product Example 137 (2 mg). LC-MS: 640.4 [M+H]⁺, ¹H NMR (400 MHz, DMSO-d6) δ 11.10 (s, 1H), 8.20 (d, J=8.8 Hz, 1H), 7.88 (d, J=5.3 Hz, 1H), 7.57-7.49 (m, 1H), 7.24 (s, 1H), 7.05 (d, J=7.0 Hz, 1H), 6.94 (d, J=8.8 Hz, 1H), 6.85 (d, J=8.5 Hz, 1H), 6.44 (s, 2H), 5.09 (dd, J=12.9, 5.5 Hz, 1H), 4.45 (s, 2H), 3.92 (s, 3H), 3.13-3.08 (m, 2H), 3.05-2.98 (m, 1H), 2.93-2.85 (m, 1H), 2.64-2.60 (m, 2H), 2.18-2.08 (m, 2H), 2.07-1.98 (m, 1H), 1.90-1.82 (m, 2H), 1.57-1.44 (m, 3H), 1.18-1.03 (m, 3H).

### Example 138

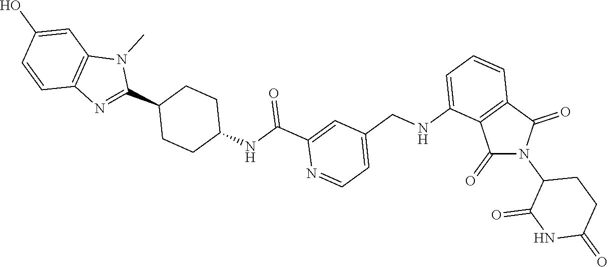 [SMILES: Cn1c2cc(O)ccc2nc1[C@H]1CC[C@H](NC(=O)c2cc(CNc3cccc4c3C(=O)N(C3CCC(=O)NC3=O)C4=O)ccn2)CC1]

### Step 1

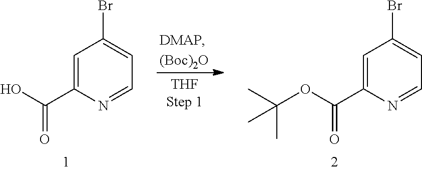

The solution of compound 1 (50 g, 247.5 mmol), DMAP (15.1 g, 123.8 mmol) and Boc₂O (134.9 g, 618.8 mmol) in THF (500 mL) was stirred at room temperature for 3 h. The solution was diluted with water (1000 mL) and DCM (1000 mL). The organic layer was separated and the inorganic layer was extracted with DCM (1000 mL×2). The organic layer were combined and washed with a solution of NaHCO₃ (500 mL×3) and brine (500 mL), dried over Na₂SO₄, filtered and concentrated to give a residue, which was purified by flash chromatography (Petroleum ether:Ethyl acetate=5:1) to give the compound 2 (50 g, 78.62% yield) as a white solid. LC-MS: 258.0 [M+1H]⁺

### Step 2

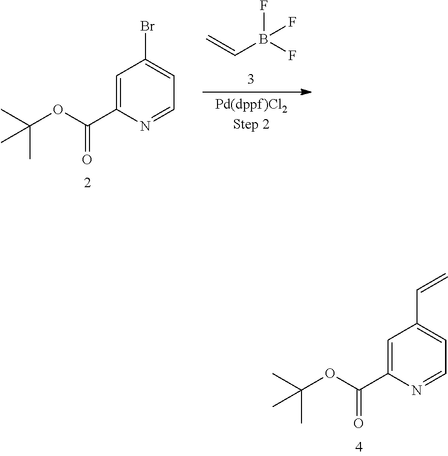

The mixture of compound 2 (20 g, 77.8 mmol), compound 3 (20.9 g, 155.6 mmol), Pd(dppf)Cl₂ (5.6 g, 7.9 mmol) and K₂CO₃ (21.5 g, 155.6 mmol) in Dioxane (200 mL) and water (10 mL) was stirred at 100° C. for 4 h under N₂ atmosphere. The solution was diluted with 200 mL of water and the solution was extracted with Ethyl acetate (200 mL×3). The organic layers were washed with brine (200 mL×3), dried over Na₂SO₄, filtered and concentrated to give a residue, which was purified by flash chromatography (Petroleum ether:Ethyl acetate=5:1) to give the compound 4 (10.4 g, 65.20% yield) as a white solid. LC-MS: 206.1 [M+H]⁺

### Step 3

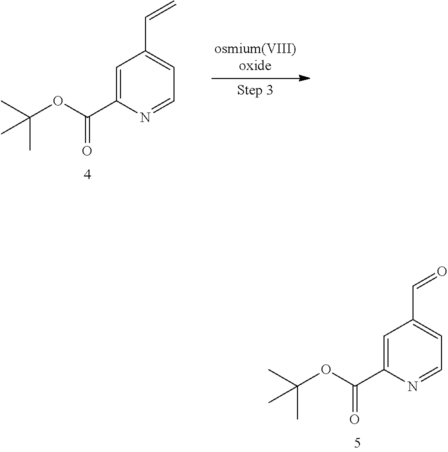

To a solution of compound 4 (10.4 g, 50.7 mmol) in Dioxane (200 mL) was added Sodium periodate (32.6 g, 152.2 mmol) and a solution of Osmium (VIII) oxide (747 mg, 2.0 mmol) in H₂O (20 mL). The solution was stirred at room temperature for 2 h. The solution was diluted with water (100 mL) and the solution was extracted with Ethyl acetate (100 mL×3). The organic layers were washed with brine (100 mL×3), dried over Na₂SO₄, filtered and concentrated to give a residue, which was purified by flash chromatography (Petroleum ether:Ethyl acetate=1:1) to give the compound 5 (6.4 g, 60.95% yield) as a yellow solid.

### Step 4

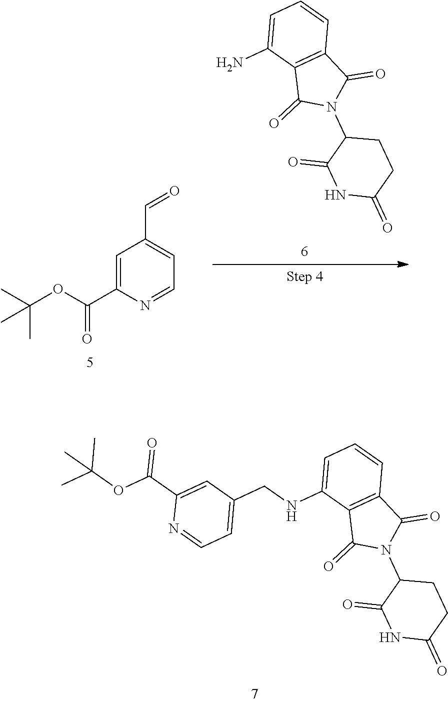 [SMILES: CC(C)(C)OC(=O)c1cc(C=O)ccn1 ; Nc1cccc2c1C(=O)N(C1CCC(=O)NC1=O)C2=O ; CC(C)(C)OC(=O)c1cc(CNc2cccc3c2C(=O)N(C2CCC(=O)NC2=O)C3=O)ccn1]

The mixture of compound 5 (3.0 g, 14.5 mmol), compound 6 (4.0 g, 14.5 mmol), dibutyldichlorotin (5.3 g, 17.4 mmol) and Phenylsilane (2.4 g, 21.8 mmol) in THF (30 mL) was stirred at 70° C. for 2 h. The solvent was removed under reduced pressure to give a residue, which was purified by flash chromatography (Petroleum ether:Ethyl acetate=1:1) to give the compound 7 (0.8 g, 11.90% yield) as a yellow solid. LC-MS: 465.1 [M+H]⁺

### Step 5

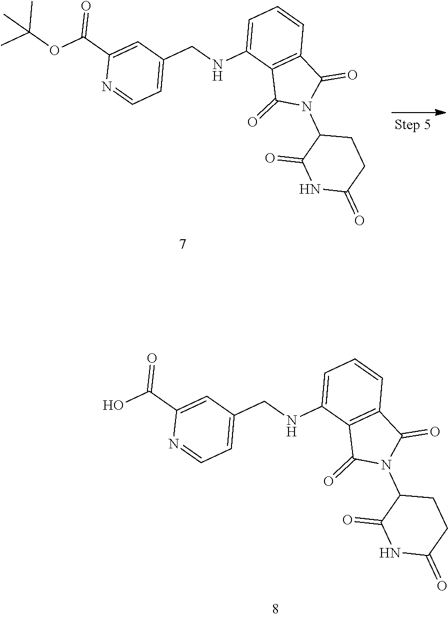 [SMILES: CC(C)(C)OC(=O)c1cc(CNc2cccc3c2C(=O)N(C2CCC(=O)NC2=O)C3=O)ccn1 ; O=C1CCC(N2C(=O)c3cccc(NCc4ccnc(C(=O)O)c4)c3C2=O)C(=O)N1]

The solution of compound 7 (800 mg, 1.72 mmol) in DCM (10 mL) and TFA (10 mL) was stirred at room temperature for 16 h. The solvent was removed under reduced pressure to give the crude compound 8 (600 mg) as a yellow solid. LC-MS: 409.0 [M+H]⁺. The crude product was used for the next step without further purification.

### Step 6

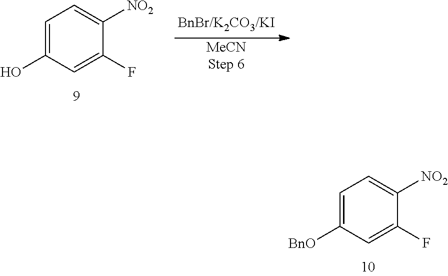 [SMILES: O=[N+]([O-])c1ccc(O)cc1F ; "MeCNStep 6" ; O=[N+]([O-])c1ccc(OCc2ccccc2)cc1F]

The mixture of compound 9 (50 g, 318.3 mmol), BnBr (57.2 g, 334.2 mmol), K₂CO₃ (46.1 g, 334.2 mmol) and KI (5.28 g, 31.83 mmol) in acetonitrile (500 mL) was stirred at 80° C. for 1 h. The solution was cooled to room temperature and diluted with water (500 mL) and the solution was extracted Ethyl acetate (500 mL×3). The organic layers were washed with brine (500 mL×3), dried over Na₂SO₄, filtered and concentrated to give a residue, which was purified by flash chromatography (Petroleum ether:Ethyl acetate=5:1) to give the compound 10 (60 g, 76.34% yield) as a white solid.

### Step 7

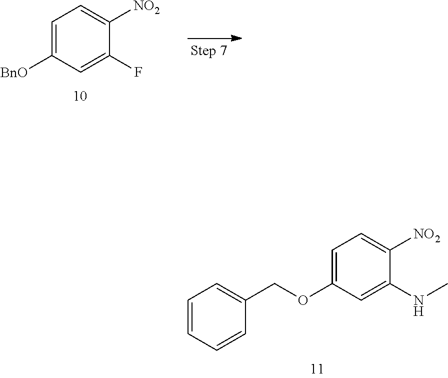 [SMILES: O=[N+]([O-])c1ccc(OCc2ccccc2)cc1F ; CNc1cc(OCc2ccccc2)ccc1[N+](=O)[O-]]

The solution of compound 10 (60 g, 242.9 mmol) and methanamine (40% in water, 500 mL) in NMP (600 mL) was stirred at 50° C. for 16 h. The solution was cooled to room temperature and diluted with water (600 mL) and the solution was extracted Ethyl acetate (600 mL×3). The organic layers were washed with brine (500 mL), dried over Na₂SO₄, filtered and concentrated to give a residue, which was purified by flash chromatography (Petroleum ether:Ethyl acetate=5:1) to give the compound 11 (54 g, 86.17% yield) as a white solid. LC-MS: 259.1 [M+H]⁺

### Step 8

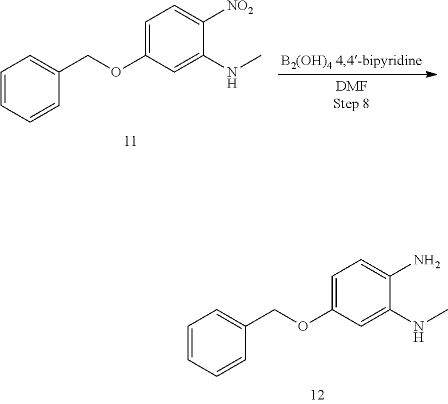 [SMILES: CNc1cc(OCc2ccccc2)ccc1[N+](=O)[O-] ; CNc1cc(OCc2ccccc2)ccc1N]

To s solution of compound 8 (30 g, 116.3 mmol) in DMF (300 mL) was added 4,4′bipyridine (1.8 g, 11.6 mmol) and B₂(OH)₄ (20.7 g, 232.6 mmol) at 0° C. The solution was stirred at room temperature for 5 min. The solution was diluted with water (300 mL) and the solution was extracted Ethyl acetate (300 mL×3). The organic layers were washed with brine (300 mL), dried over Na₂SO₄, filtered and concentrated to give a residue, which was purified by flash chromatography (Petroleum ether:Ethyl acetate=5:1) to give the compound 12 (9 g, 33.96% yield) as a white solid. LC-MS: 229.2 [M+H]⁺

### Step 9

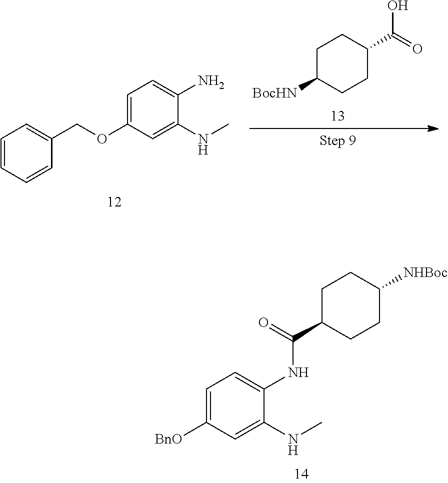 [SMILES: CNc1cc(OCc2ccccc2)ccc1N ; CC(C)(C)OC(=O)N[C@H]1CC[C@H](C(=O)O)CC1 ; CNc1cc(OCc2ccccc2)ccc1NC(=O)[C@H]1CC[C@H](NC(=O)OC(C)(C)C)CC1]

To a solution of compound 12 (3 g, 13.16 mmol) and compound 13 (3.2 g, 13.16 mmol) in DMF (30 mL) was added NMI (4.3 g, 52.64 mmol and TCFH (4.42 g, 15.79 mmol). The solution was stirred at room temperature for 16 h. The solution was diluted with water (100 mL) and the solution was extracted Ethyl acetate (100 mL×3). The organic layers were washed with brine (100 mL), dried over Na₂SO₄, filtered and concentrated to give a residue, which was purified by flash chromatography (Petroleum ether:Ethyl acetate=1:1) to give the compound 14 (2.8 g, 46.98% yield) as a white solid. LC-MS: 454.2 [M+H]⁺

### Step 10

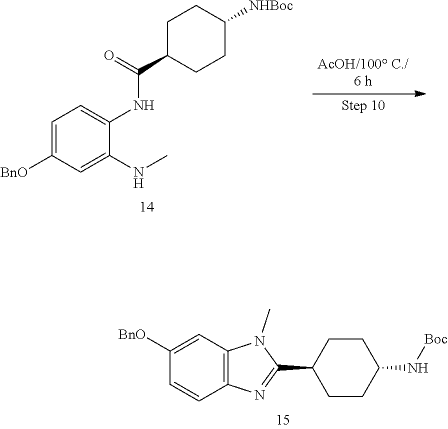 [SMILES: CNc1cc(OCc2ccccc2)ccc1NC(=O)[C@H]1CC[C@H](NC(=O)OC(C)(C)C)CC1 ; Cn1c2cc(OCc3ccccc3)ccc2nc1[C@H]1CC[C@H](NC(=O)OC(C)(C)C)CC1]

The solution of compound 14 (2.8 g, 6.18 mmol) in AcOH (30 mL) was stirred at 100° C. for 16 h. The solvent was removed under reduced pressure to give a residue, which was purified by flash chromatography (Petroleum ether:Ethyl acetate=1:1) to give the compound 15 (2.0 g, 74.35% yield) as a white solid. LC-MS: 436.2 [M+H]⁺

¹H NMR (400 MHz, DMSO-d6) δ 7.48 (d, J=7.4 Hz, 2H), 7.42-7.21 (m, 3H), 7.33 (t, J=7.3 Hz, 1H), 7.16 (d, J=2.1 Hz, 1H), 6.83 (dd, J=8.7, 2.4 Hz, 2H), 5.14 (s, 2H), 3.70 (s, 3H), 3.30-3.25 (m, 1H), 2.93-2.77 (m, 1H), 2.05-1.78 (m, 4H), 1.71-1.53 (m, 2H), 1.48-1.26 (m, 111H).

### Step 11

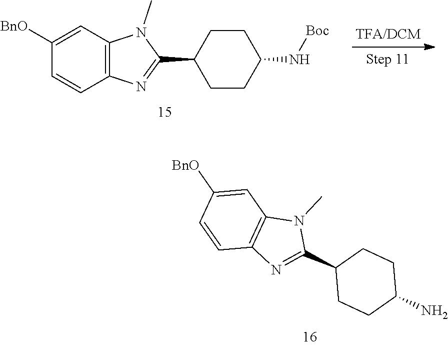 [SMILES: Cn1c2cc(OCc3ccccc3)ccc2nc1[C@H]1CC[C@H](NC(=O)OC(C)(C)C)CC1 ; Cn1c2cc(OCc3ccccc3)ccc2nc1[C@H]1CC[C@H](N)CC1]

The solution of compound 15 (2 g, 4.60 mmol) in DCM (20 mL) and TFA (8 mL) was stirred at room temperature for 16 h. The solvent was removed under reduced pressure to give the crude compound 16 (1.7 g, crude) as a yellow solid. LC-MS: 336.2 [M+H]⁺. The crude product was used for the next step without further purification.

### Step 12

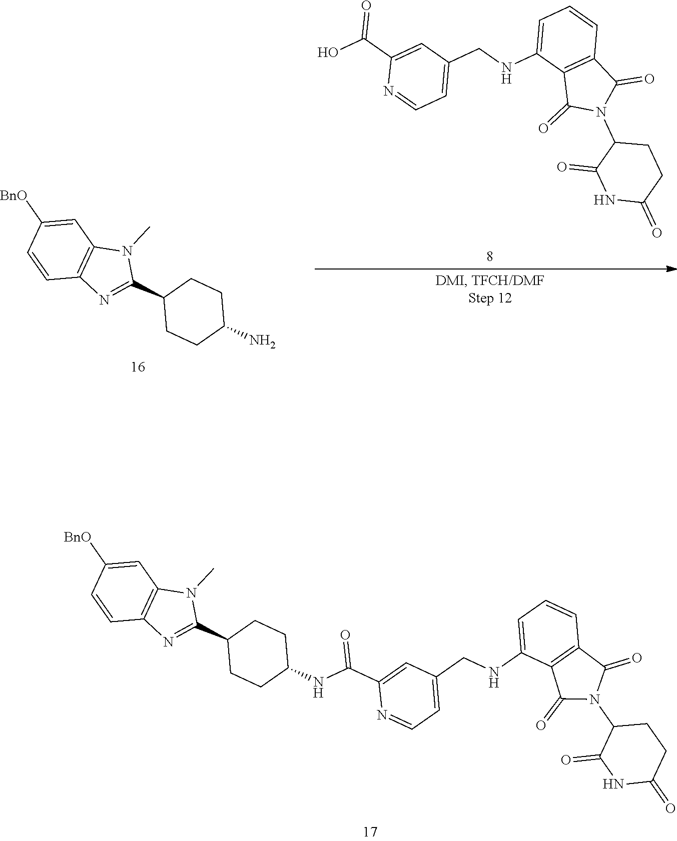 [SMILES: Cn1c2cc(OCc3ccccc3)ccc2nc1[C@H]1CC[C@H](N)CC1 ; O=C1CCC(N2C(=O)c3cccc(NCc4ccnc(C(=O)O)c4)c3C2=O)C(=O)N1 ; Cn1c2cc(OCc3ccccc3)ccc2nc1[C@H]1CC[C@H](NC(=O)c2cc(CNc3cccc4c3C(=O)N(C3CCC(=O)NC3=O)C4=O)ccn2)CC1]

To a solution of compound 16 (800 mg, 2.39 mmol) and compound 8 (974 mg, 2.39 mmol) in DMF (10 mL) was added NMI (784 mg, 9.56 mmol) and TCFH (803 mg, 2.87 mmol). The solution was stirred at room temperature for 16 h. The solution was diluted with water (50 mL) and the solution was extracted Ethyl acetate (50 mL×3). The organic layers were washed with brine (50 mL), dried over Na₂SO₄, filtered and concentrated to give a residue, which was purified by flash chromatography (DCM:MeOH=5:1) to give the compound 17 (650 mg, 37.55% yield) as a yellow solid. LC-MS: 726.2 [M+H]⁺

### Step 13

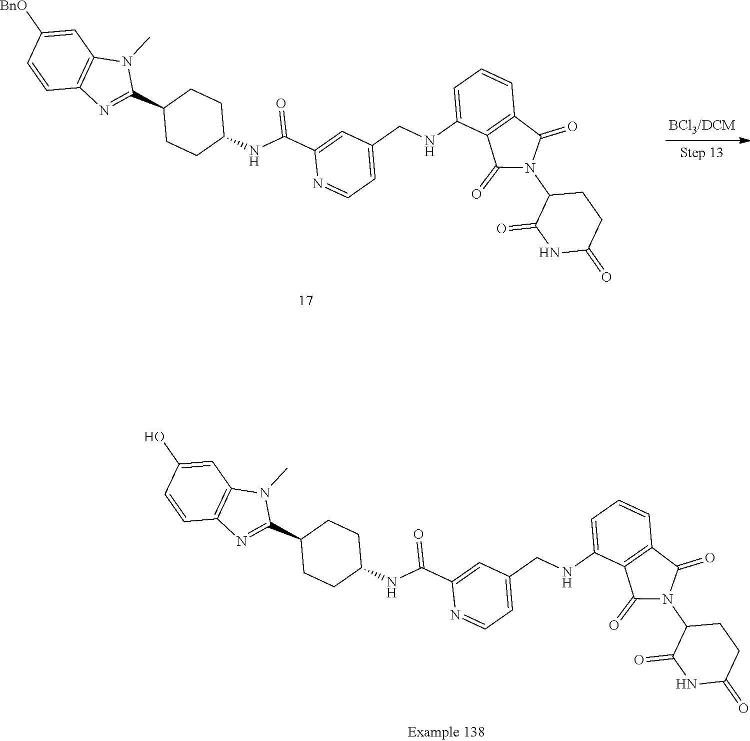 [SMILES: Cn1c2cc(OCc3ccccc3)ccc2nc1[C@H]1CC[C@H](NC(=O)c2cc(CNc3cccc4c3C(=O)N(C3CCC(=O)NC3=O)C4=O)ccn2)CC1 ; Cn1c2cc(O)ccc2nc1[C@H]1CC[C@H](NC(=O)c2cc(CNc3cccc4c3C(=O)N(C3CCC(=O)NC3=O)C4=O)ccn2)CC1]

To a solution of compound 17 (650 mg, 0.90 mmol) in DCM (10 mL) was added BCl₃ (268 mg, 1.8 mmol) dropwise. The solution was stirred at room temperature for 3 h. The solution was quenched with MeOH (8 mL). The solution was concentrated under reduced pressure to give a residue, which was purified by Prep-HPLC to give the desired compound Example 138 (230 mg). LC-MS: 636.4 [M+H]^{+ 1}H NMR (400 MHz, DMSO-d6) δ 11.12 (s, 1H), 9.12 (s, 1H), 8.59-8.54 (m, 2H), 8.05 (s, 1H), 7.60-7.55 (m, 1H), 7.52-7.46 (m, 2H), 7.31 (d, J=8.6 Hz, 1H), 7.05 (d, J=7.0 Hz, 1H), 6.87 (d, J=8.6 Hz, 1H), 6.75 (d, J=2.2 Hz, 1H), 6.62 (dd, J=8.6, 2.3 Hz, 1H), 5.10 (dd, J=12.8, 5.4 Hz, 1H), 4.71 (d, J=6.3 Hz, 2H), 3.92-3.79 (m, 1H), 3.65 (s, 3H), 2.98-2.80 (m, 2H), 2.68-2.52 (m, 2H), 2.15-1.89 (m, 5H), 1.75-1.61 (m, 4H).

### Examples 139-150

Examples 139-150 can be synthesized similarly according to the methods described herein.

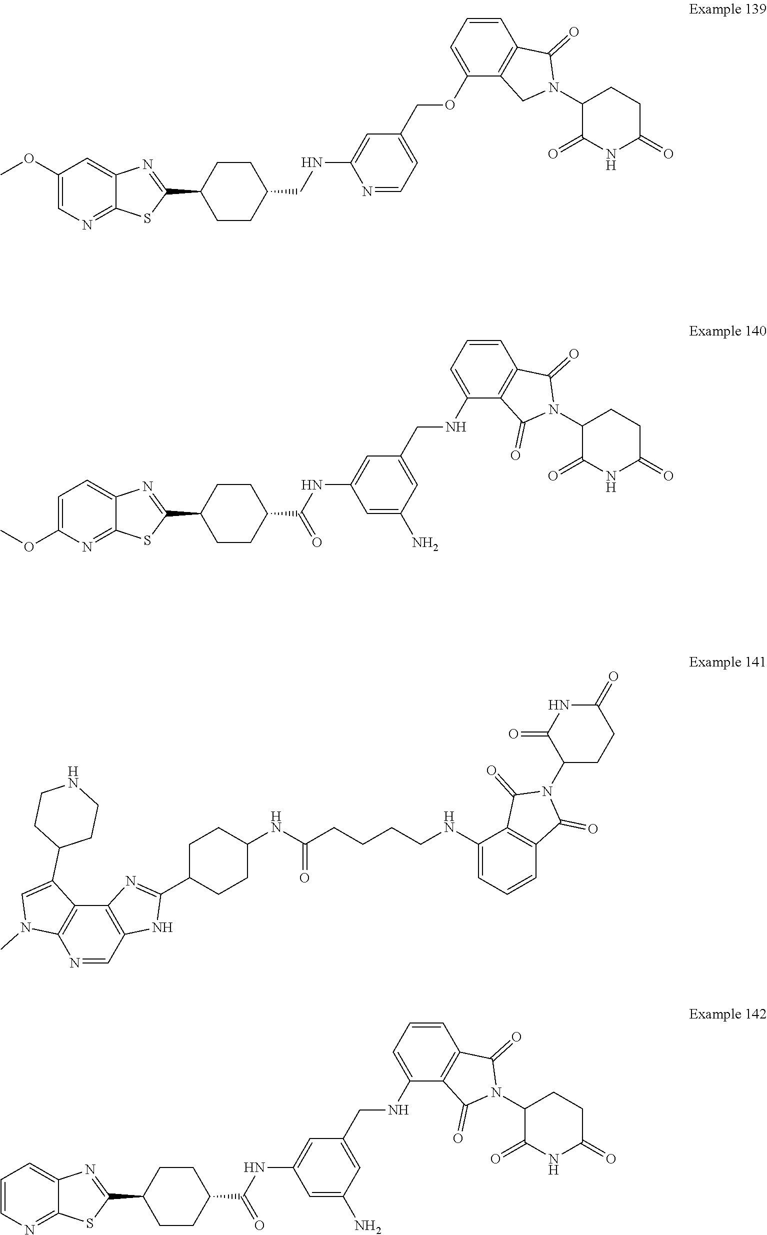 [SMILES: COc1cnc2sc([C@H]3CC[C@H](CNc4cc(COc5cccc6c5CN(C5CCC(=O)NC5=O)C6=O)ccn4)CC3)nc2c1 ; COc1ccc2nc([C@H]3CC[C@H](C(=O)Nc4cc(N)cc(CNc5cccc6c5C(=O)N(C5CCC(=O)NC5=O)C6=O)c4)CC3)sc2n1 ; Cn1cc(C2CCNCC2)c2c3nc(C4CCC(NC(=O)CCCCNc5cccc6c5C(=O)N(C5CCC(=O)NC5=O)C6=O)CC4)[nH]c3cnc21 ; Nc1cc(CNc2cccc3c2C(=O)N(C2CCC(=O)NC2=O)C3=O)cc(NC(=O)[C@H]2CC[C@H](c3nc4cccnc4s3)CC2)c1]

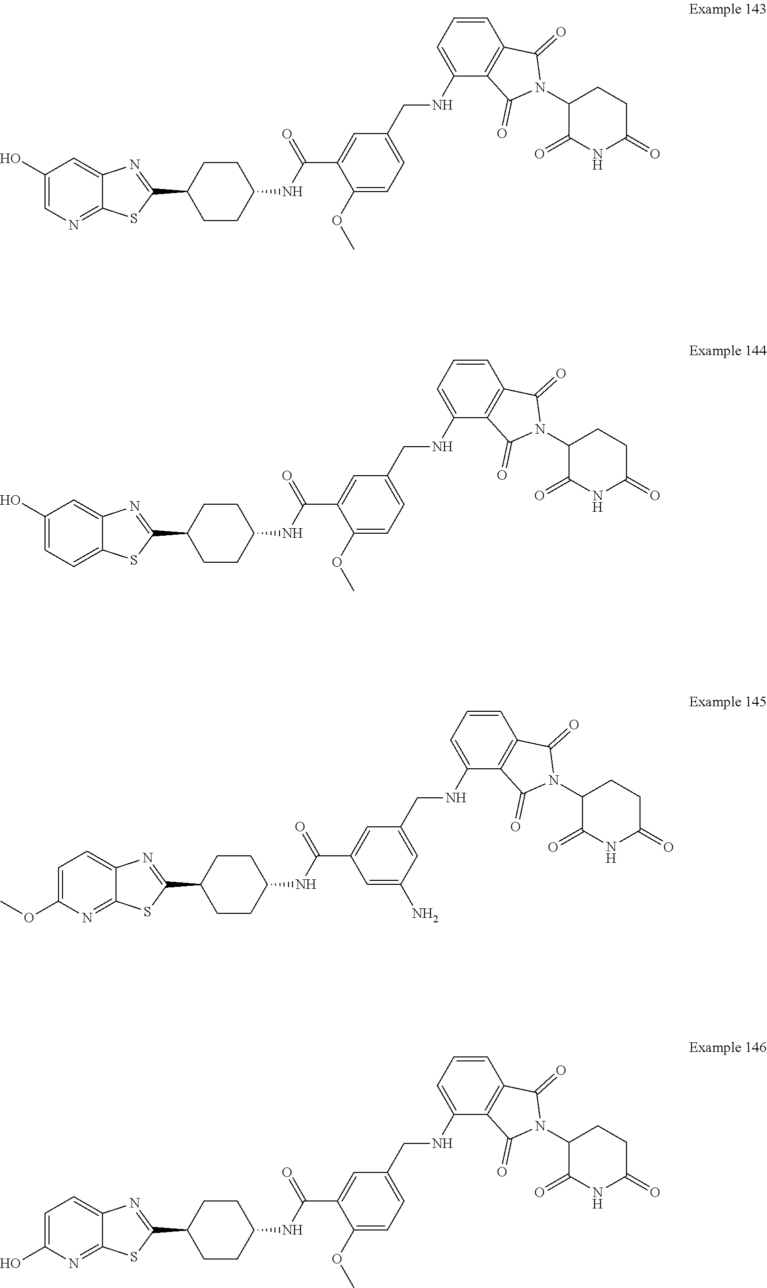 [SMILES: COc1ccc(CNc2cccc3c2C(=O)N(C2CCC(=O)NC2=O)C3=O)cc1C(=O)N[C@H]1CC[C@H](c2nc3cc(O)cnc3s2)CC1 ; COc1ccc(CNc2cccc3c2C(=O)N(C2CCC(=O)NC2=O)C3=O)cc1C(=O)N[C@H]1CC[C@H](c2nc3cc(O)ccc3s2)CC1 ; COc1ccc2nc([C@H]3CC[C@H](NC(=O)c4cc(N)cc(CNc5cccc6c5C(=O)N(C5CCC(=O)NC5=O)C6=O)c4)CC3)sc2n1 ; COc1ccc(CNc2cccc3c2C(=O)N(C2CCC(=O)NC2=O)C3=O)cc1C(=O)N[C@H]1CC[C@H](c2nc3ccc(O)nc3s2)CC1]

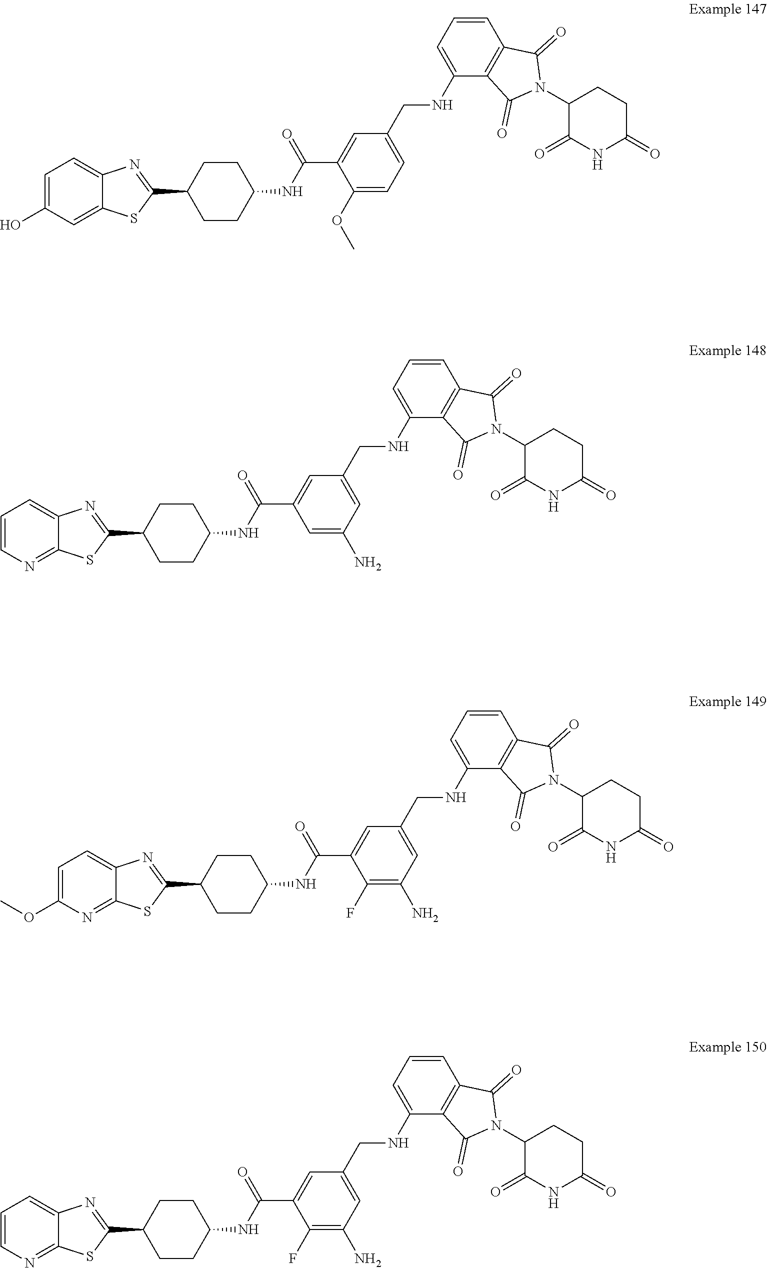 [SMILES: COc1ccc(CNc2cccc3c2C(=O)N(C2CCC(=O)NC2=O)C3=O)cc1C(=O)N[C@H]1CC[C@H](c2nc3ccc(O)cc3s2)CC1 ; Nc1cc(CNc2cccc3c2C(=O)N(C2CCC(=O)NC2=O)C3=O)cc(C(=O)N[C@H]2CC[C@H](c3nc4cccnc4s3)CC2)c1 ; COc1ccc2nc([C@H]3CC[C@H](NC(=O)c4cc(CNc5cccc6c5C(=O)N(C5CCC(=O)NC5=O)C6=O)cc(N)c4F)CC3)sc2n1 ; Nc1cc(CNc2cccc3c2C(=O)N(C2CCC(=O)NC2=O)C3=O)cc(C(=O)N[C@H]2CC[C@H](c3nc4cccnc4s3)CC2)c1F]

### Activity Test Example 1: HL60 Cell Killing Experiment

### 1. Materials

HL60 cell lines were purchased from ATCC (Cat #ATCC® CCL-240™), IMDM media were purchased from ATCC (Cat #ATCC® 30-2005™), FBS was purchased from Gibco (Cat #10091148), celltiter-glo luminescent cell viability assay was purchased from promega (Cat #G7572), and 96-well plates were purchased from corning (Cat #3610).

### 2. Method

The effect of compounds on the growth of HL60 cells was determined by measuring cell viability. Firstly, cells in the logarithmic growth phase were collected and centrifuged, and the culture supernatant was discarded. The centrifuged cells were resuspended in a fresh complete medium, and the cell density was adjusted to 5×10⁴ cells/mL. The resuspended cells were inoculated into two opaque 96-well cell culture plates at 100 μL of cell suspension per well, and the cell culture plates were incubated in a cell incubator at 37° C. for 4 h. After 4 h, dilutions (20 μL) of compounds with different concentrations were added. After one of the cell plates was cooled to room temperature, 60 μL of CTG reagent was added, and the luminescence values were read on the instrument as G0. The other one was incubated in a cell incubator for 48 h. After 48 h, 60 μL of CTG reagent was added, and the luminescence values were read on the instrument as G3.

### 3. Data Analysis

$$\% Proliferation = ( test compound well G 3 - G 0 average value ) / ( DMSO control well G 3 average value - G 0 average value ) \times 100$$

According to the corresponding proliferation rate and concentration of each gradient concentration well, a gradient curve of cell proliferation was fitted using XLFit software, and the GI50 values of compounds were calculated.

### 4. Results

According to the above experimental method, GI50 values of the compounds prepared in the above examples were measured as shown in the table below:

TABLE 2
Experimental results

| No. | GI50/nM | No. | GI50/nM | No. | GI50/nM | No. | GI50/nM |
|---|---|---|---|---|---|---|---|
| 1 | <100 | 2 | <100 | 3 | 100 < GI50 < 1000 | 4 | 100 < GI50 < 1000 |
| 5 | <100 | 6 | <100 | 7 | 1000 < GI50 < 5000 | 8 | <100 |
| 9 | 100 < GI50 < 1000 | 10 | <100 | 11 | 1000 < GI50 < 5000 | 12 | <100 |
| 13 | <100 | 14 | <100 | 15 | <100 | 16 | <100 |
| 17 | <100 | 18 | <100 | 19 | <100 | 20 | <100 |
| 21 | <100 | 22 | <100 | 23 | <100 | 25 | <100 |
| 26 | <100 | 28 | <100 | 29 | <100 | 30 | <100 |
| 31 | 100 < GI50 < 1000 | 32 | 1000 < GI50 < 5000 | 33 | <100 | 34 | <100 |
| 35 | <100 | 36 | <100 | 37 | <100 | 38 | <100 |
| 39 | <100 | 40 | 100 < GI50 < 1000 | 41 | <100 | 42 | <100 |
| 43 | <100 | 44 | <100 | 45 | <100 | 46 | <100 |
| 47 | 1000 < GI50 < 5000 | 48 | <100 | 49 | <100 | 50 | <100 |
| 51 | <100 | 52 | <100 | 53 | <100 | 54 | <100 |
| 55 | 1000 < GI50 < 5000 | 56 | <100 | 57 | <100 | 59 | <100 |
| 60 | <100 | 61 | <100 | 62 | <100 | 63 | 100 < GI50 < 1000 |
| 64 | <100 | 65 | 1000 < GI50 < 5000 | 66 | <100 | 68 | <100 |
| 69 | <100 | 70 | <100 | 71 | <100 | 73 | <100 |
| 74 | 100 < GI50 < 1000 | 75 | <100 | 76 | 1000 < GI50 < 5000 | 77 | <100 |
| 78 | <100 | 79 | <100 | 80 | <100 | 81 | <100 |
| 82 | <100 | 83 | <100 | 84 | <100 | 85 | <100 |
| 86 | <100 | 87 | <100 | 88 | <100 | 89 | <100 |
| 90 | <100 | 91 | 100 < GI50 < 1000 | 92 | <100 | 93 | 1000 < GI50 < 5000 |
| 94 | <100 | 95 | <100 | 96 | 100 < GI50 < 1000 | 97 | <100 |
| 98 | <100 | 99 | <100 | 100 | <100 | 101 | <100 |
| 102 | <100 | 103 | <100 | 104 | 100 < GI50 < 1000 | 105 | 100 < GI50 < 1000 |
| 106 | <100 | 107 | 1000 < GI50 < 5000 | 109 | <100 | 110 | <100 |
| 111 | 1000 < GI50 < 5000 | 112 | <100 | 113 | <100 | 114 | 100 < GI50 < 1000 |
| 115 | <100 | 116 | <100 | 130 | <100 | | |

Note:
No. in the above table refers to the number of the synthesis example, and refers to the compound prepared in the corresponding example.

### Activity Test Example 2: THP-1 Cell Killing Experiment

### 1. Materials

THP-1 cell lines were purchased from ATCC (Cat #ATCC® TIB-202™), RPMI1640 media were purchased from ATCC (Cat #ATCC® 30-2001™), FBS was purchased from Gibco (Cat #10091148), celltiter-glo luminescent cell viability assay was purchased from promega (Cat #G7572), and 96-well plates were purchased from coming (Cat #3610).

### 2. Method

The effect of compounds on the growth of THP-1 cells was determined by measuring cell viability and growth rate. Firstly, cells in the logarithmic growth phase were collected and centrifuged, and the culture supernatant was discarded. The centrifuged cells were resuspended in a fresh complete medium, and the cell density was adjusted to 5×10⁴ cells/mL. The resuspended cells were inoculated into two identical opaque 96-well cell culture plates at 100 μL of cell suspension per well, and the cell culture plates were incubated in a cell incubator at 37° C. for 4 h. After 4 h, dilutions (20 μL) of compounds with different concentrations were added. After one of the cell plates was cooled to room temperature, 60 μL of CTG reagent was added, and the luminescence values were read on the instrument as G0. The other one was incubated in a cell incubator for 48 h. After 48 h, 60 μL of CTG reagent was added, and the luminescence values were read on the instrument as G3.

### 3. Data Analysis

$$\% Proliferation = ( test compound well G 3 - G 0 average value ) / ( DMSO control well G 3 average value - G 0 average value ) \times 100$$

According to the corresponding proliferation rate and concentration of each gradient concentration well, a gradient curve of cell proliferation was fitted using XLFit software, and the GI50 values of compounds were calculated.

### 4. Results

According to the above experimental method, GI50 values of the compounds prepared in the above examples were measured as shown in the table below:

TABLE 3
Experimental results

| | GI50/ | | GI50/ | | GI50/ | | GI50/ |
|---|---|---|---|---|---|---|---|
| No. | nM | No. | nM | No. | nM | No. | nM |
| 83 | 67.62 | 89 | 5.65 | 90 | 4.14 | 97 | 2.58 |

### Activity Test Example 3: Jurkat Cell Killing Experiment

### 1. Materials

Jurkat cell lines were purchased from ATCC (Cat #ATCC® TIB-152™), RPMI1640 media were purchased from ATCC (Cat #ATCC® 30-2001™), FBS was purchased from Gibco (Cat #10091148), celltiter-glo luminescent cell viability assay was purchased from promega (Cat #G7572), and 96-well plates were purchased from coming (Cat #3610).

### 2. Method

The effect of compounds on the growth of Jurkat cells was determined by measuring cell viability and growth rate. Firstly, cells in the logarithmic growth phase were collected and centrifuged, and the culture supernatant was discarded. The centrifuged cells were resuspended in a fresh complete medium, and the cell density was adjusted to 5×10⁴ cells/mL. The resuspended cells were inoculated into two identical opaque 96-well cell culture plates at 100 μL of cell suspension per well, and the cell culture plates were incubated in a cell incubator at 37° C. for 4 h. After 4 h, dilutions (20 μL) of compounds with different concentrations were added. After one of the cell plates was cooled to room temperature, 60 μL of CTG reagent was added, and the luminescence values were read on the instrument as G0. The other one was incubated in a cell incubator for 48 h. After 48 h, 60 μL of CTG reagent was added, and the luminescence values were read on the instrument as G3.

### 3. Data Analysis

$$\% Proliferation = ( test compound well G 3 - G 0 average value ) / ( DMSO control well G 3 average value - G 0 average value ) \times 100$$

According to the corresponding proliferation rate and concentration of each gradient concentration well, a gradient curve of cell proliferation was fitted using XLFit software, and the GI50 values of compounds were calculated.

### 4. Results

According to the above experimental method, GI50 values of the compounds prepared in the above examples were measured as shown in the table below:

TABLE 4
Experimental results

| | GI50/ | | GI50/ | | GI50/ | | GI50/ |
|---|---|---|---|---|---|---|---|
| No. | nM | No. | nM | No. | nM | No. | nM |
| 16 | 4.07 | 26 | 2.24 | 39 | 1.73 | 75 | 0.40 |
| 81 | 11.38 | 89 | 0.50 | 94 | 2.51 | 105 | 1.30 |

### Activity Test Example 4: SU-DHL-2 Cell Killing Experiment

### 1. Materials

SU-DHL-2 cell lines were purchased from ATCC (Cat #ATCC® CRL-2956™), RPMI1640 media were purchased from ATCC (Cat #ATCC® 30-2001™), FBS was purchased from Gibco (Cat #10091148), celltiter-glo luminescent cell viability assay was purchased from promega (Cat #G7572), and 96-well plates were purchased from coming (Cat #3610).

### 2. Method

The effect of compounds on the growth of SU-DHL-2 cells was determined by measuring cell viability and growth rate. Firstly, cells in the logarithmic growth phase were collected and centrifuged, and the culture supernatant was discarded. The centrifuged cells were resuspended in a fresh complete medium, and the cell density was adjusted to 5×10⁴ cells/mL. The resuspended cells were inoculated into two identical opaque 96-well cell culture plates at 100 μL of cell suspension per well, and the cell culture plates were incubated in a cell incubator at 37° C. for 4 h. After 4 h, dilutions (20 μL) of compounds with different concentrations were added. After one of the cell plates was cooled to room temperature, 60 μL of CTG reagent was added, and the luminescence values were read on the instrument as G0. The other one was incubated in a cell incubator for 48 h. After 48 h, 60 μL of CTG reagent was added, and the luminescence values were read on the instrument as G3.

### 3. Data Analysis

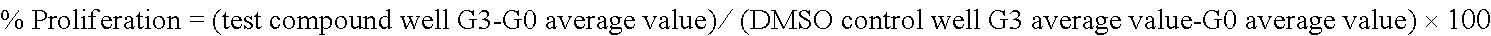

According to the corresponding proliferation rate and concentration of each gradient concentration well, a gradient curve of cell proliferation was fitted using XLFit software, and the GI50 values of compounds were calculated.

### 4. Results

According to the above experimental method, GI50 values of the compounds prepared in the above examples were measured as shown in the table below:

TABLE 5
Experimental results

| | GI50/ | | GI50/ | | GI50/ | | GI50/ |
|---|---|---|---|---|---|---|---|
| No. | nM | No. | nM | No. | nM | No. | nM |
| 70 | 22.29 | 75 | 0.93 | 81 | 5.62 | 89 | 1.42 |
| 97 | 1.51 | | | | | | |

### Activity Test Example 5: Hep3B Cell Killing Experiment

### 1. Materials

Hep3B cell lines were purchased from ATCC (Cat #ATCC® HB-8064™), EMEM media were purchased from ATCC (Cat #ATCC® 30-2003™), FBS was purchased from Gibco (Cat #10091148), celltiter-glo luminescent cell viability assay was purchased from promega (Cat #G7572), and 96-well plates were purchased from coming (Cat #3610).

### 2. Method

The effect of compounds on the growth of Hep3B cells was determined by measuring cell viability and growth rate. Firstly, the cells were digested with pancreatin, and the digestion was then terminated with medium. The cells were centrifuged, and the supernatant was discarded. The centrifuged cells were resuspended in a fresh complete medium, and the cell density was adjusted to 5×10⁴ cells/mL. The resuspended cells were inoculated into two identical opaque 96-well cell culture plates at 100 μL of cell suspension per well, and the cell culture plates were incubated in a cell incubator at 37° C. overnight. The next day, dilutions (20 μL) of compounds with different concentrations were added. After one of the cell plates was cooled to room temperature, 60 μL of CTG reagent was added. The luminescence values were read on the instrument as G0. The other one was incubated in a cell incubator for 48 h. After 48 h, 60 μL of CTG reagent was added, and the luminescence values were read on the instrument as G3.

### 3. Data Analysis

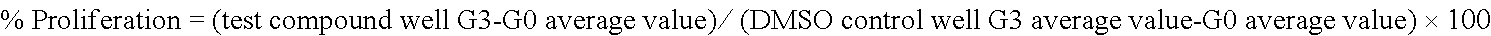

According to the corresponding proliferation rate and concentration of each gradient concentration well, a gradient curve of cell proliferation was fitted using XLFit software, and the GI50 values of compounds were calculated.

### 4. Results

According to the above experimental method, GI50 values of the compounds prepared in the above examples were measured as shown in the table below:

TABLE 6
Experimental results

| | GI50/ | | GI50/ | | GI50/ | | GI50/ |
|---|---|---|---|---|---|---|---|
| No. | nM | No. | nM | No. | nM | No. | nM |
| 10 | 6.91 | 16 | 1.36 | 26 | 2.70 | 39 | 0.99 |
| 61 | 1.48 | 70 | 5.21 | 83 | 7.88 | 97 | 0.46 |

### Activity Test Example 6: NCI-H929 Cell Killing Experiment

### 1. Materials

NCI-H929 cell lines were purchased from ATCC (Cat #ATCC® CRL-9068™), RPMI1640 media were purchased from ATCC (Cat #ATCC® 30-2001™), FBS was purchased from Gibco (Cat #10091148), celltiter-glo luminescent cell viability assay was purchased from promega (Cat #G7572), and 96-well plates were purchased from coming (Cat #3610).

2. Method The effect of compounds on the growth of NCI-H929 cells was determined by measuring cell viability and growth rate. Firstly, cells in the logarithmic growth phase were collected and centrifuged, and the culture supernatant was discarded. The centrifuged cells were resuspended in a fresh complete medium, and the cell density was adjusted to 5×10⁴ cells/mL. The resuspended cells were inoculated into two identical opaque 96-well cell culture plates at 100 μL of cell suspension per well, and the cell culture plates were incubated in a cell incubator at 37° C. for 4 h. After 4 h, dilutions (20 μL) of compounds with different concentrations were added. After one of the cell plates was cooled to room temperature, 60 μL of CTG reagent was added, and the luminescence values were read on the instrument as G0. The other one was incubated in a cell incubator for 48 h. After 48 h, 60 μL of CTG reagent was added, and the luminescence values were read on the instrument as G3.

### 3. Data Analysis

$$\% Proliferation = ( test compound well G 3 - G 0 average value ) / ( DMSO control well G 3 average value - G 0 average value ) \times 100$$

According to the corresponding proliferation rate and concentration of each gradient concentration well, a gradient curve of cell proliferation was fitted using XLFit software, and the GI50 values of compounds were calculated.

### 4. Results

According to the above experimental method, GI50 values of the compounds prepared in the above examples were measured as shown in the table below:

TABLE 7
Experimental results

| | GI50/ | | GI50/ | | GI50/ | | GI50/ |
|---|---|---|---|---|---|---|---|
| No. | nM | No. | nM | No. | nM | No. | nM |
| 10 | 19.61 | 16 | 0.96 | 61 | 1.08 | 70 | 1.63 |
| 81 | 0.33 | 83 | 5.49 | 90 | 0.93 | | |

### Activity Test Example 7: Toledo Cell Killing Experiment

### 1. Materials

Toledo cell lines were purchased from ATCC (Cat #ATCC® CRL-2631™), RPMI1640 media were purchased from ATCC (Cat #ATCC® 30-2001™), FBS was purchased from Gibco (Cat #10091148), celltiter-glo luminescent cell viability assay was purchased from promega (Cat #G7572), and 96-well plates were purchased from coming (Cat #3610).

### 2. Method

The effect of compounds on the growth of Toledo cells was determined by measuring cell viability and growth rate. Firstly, cells in the logarithmic growth phase were collected and centrifuged, and the culture supernatant was discarded. The centrifuged cells were resuspended in a fresh complete medium, and the cell density was adjusted to 5×10⁴ cells/mL. The resuspended cells were inoculated into two identical opaque 96-well cell culture plates at 100 μL of cell suspension per well, and the cell culture plates were incubated in a cell incubator at 37° C. for 4 h. After 4 h, dilutions (20 μL) of compounds with different concentrations were added. After one of the cell plates was cooled to room temperature, 60 μL of CTG reagent was added, and the luminescence values were read on the instrument as G0. The other one was incubated in a cell incubator for 48 h. After 48 h, 60 μL of CTG reagent was added, and the luminescence values were read on the instrument as G3.

### 3. Data Analysis

$$\% Proliferation = ( test compound well G 3 - G 0 average value ) / ( DMSO control well G 3 average value - G 0 average value ) \times 100$$

According to the corresponding proliferation rate and concentration of each gradient concentration well, a gradient curve of cell proliferation was fitted using XLFit software, and the GI50 values of compounds were calculated.

### 4. Results

According to the above experimental method, GI50 values of the compounds prepared in the above examples were measured as shown in the table below:

TABLE 8
Experimental results

| | GI50/ | | GI50/ | | GI50/ | | GI50/ |
|---|---|---|---|---|---|---|---|
| No. | nM | No. | nM | No. | nM | No. | nM |
| 26 | 11.72 | 39 | 3.58 | 61 | 5.31 | 75 | 0.45 |
| 81 | 4.03 | 113 | 1.53 | | | | |

### Activity Test Example 8: Raji Cell Killing Experiment

### 1. Materials

Raji cell lines were purchased from ATCC (Cat #ATCC® CCL-86™), RPMI1640 media were purchased from ATCC (Cat #ATCC® 30-2001™), FBS was purchased from Gibco (Cat #10091148), celltiter-glo luminescent cell viability assay was purchased from promega (Cat #G7572), and 96-well plates were purchased from coming (Cat #3610).

### 2. Method

The effect of compounds on the growth of Raji cells was determined by measuring cell viability and growth rate. Firstly, cells in the logarithmic growth phase were collected and centrifuged, and the culture supernatant was discarded. The centrifuged cells were resuspended in a fresh complete medium, and the cell density was adjusted to 5×10⁴ cells/mL. The resuspended cells were inoculated into two identical opaque 96-well cell culture plates at 100 μL of cell suspension per well, and the cell culture plates were incubated in a cell incubator at 37° C. for 4 h. After 4 h, dilutions (20 μL) of compounds with different concentrations were added. After one of the cell plates was cooled to room temperature, 60 μL of CTG reagent was added, and the luminescence values were read on the instrument as G0. The other one was incubated in a cell incubator for 48 h. After 48 h, 60 μL of CTG reagent was added, and the luminescence values were read on the instrument as G3.

### 3. Data Analysis

$$\% Proliferation = ( test compound well G 3 - G 0 average value ) / ( DMSO control well G 3 average value - G 0 average value ) \times 100$$

According to the corresponding proliferation rate and concentration of each gradient concentration well, a gradient curve of cell proliferation was fitted using XLFit software, and the GI50 values of compounds were calculated.

### 4. Results

According to the above experimental method, GI50 values of the compounds prepared in the above examples were measured as shown in the table below:

TABLE 9
Experimental results

| | GI50/ | | GI50/ | | GI50/ | | GI50/ |
|---|---|---|---|---|---|---|---|
| No. | nM | No. | nM | No. | nM | No. | nM |
| 16 | 362.02 | 75 | 1.79 | 90 | 5.88 | 97 | 3.43 |

### Activity Test Example 9: Study on Degradation of GSPT1 by the Compounds

### 1. Materials

GSPT1/HEK293 hibit cell lines were constructed by Shanghai BioDuro Co., Ltd., HEK293 cell lines were purchased from ATCC (Cat #ATCC® CRL-1573™), DMEM media were purchased from ATCC (Cat #ATCC® 30-2002™), FBS was purchased from Gibco (Cat #10091148), Nano Glo HiBiT Lytic Detection System was purchased from Promega (Cat #N3040), and 96-well plates were purchased from coming (Cat #3610).

### 2. Method

The degradation of GSPT1 by the compounds was determined by measuring the intensity of hibit signals in cells. Firstly, the cells were digested with pancreatin, and the digestion was then terminated with medium. The cells were centrifuged, and the supernatant was discarded. The centrifuged cells were resuspended in a fresh complete medium, and the cell density was adjusted to 2.5×10⁴ cells/mL. The resuspended cells were inoculated into an opaque 96-well cell culture plate at 100 μL of cell suspension per well, and the cell culture plate was incubated in a cell incubator at 37° C. overnight. The next day, dilutions (20 μL) of compounds with different concentrations were added, and the plate was incubated in a cell incubator for 6 h. After 6 h, 60 μL of Nano Glo HiBiT Lytic Detection System reagent was added and the luminescence values were read on the instrument.

### 3. Data Analysis

$$Degradation rate = 100 - ( value of test compound well / average value of DMSO control wells ) \times 100$$

According to the corresponding degradation rate and concentration of each gradient concentration well, a gradient curve of degradation rate was fitted using XLFit software, and the DC50 values of compounds were calculated.

### 4. Results

According to the above experimental method, DC50 values of the compounds prepared in the above examples were measured as shown in the table below:

TABLE 10
Experimental results

| No. | DC50/nM | No. | DC50/nM | No. | DC50/nM | No. | DC50/nM |
|---|---|---|---|---|---|---|---|
| 1 | <100 | 2 | <100 | 3 | <100 | 4 | <100 |
| 5 | <100 | 6 | <100 | 8 | <100 | 9 | <100 |
| 10 | <100 | 12 | <100 | 13 | <100 | 14 | <100 |
| 15 | <100 | 16 | <100 | 17 | <100 | 18 | <100 |
| 19 | <100 | 21 | <100 | 22 | <100 | 23 | <100 |
| 25 | <100 | 26 | <100 | 29 | <100 | 30 | <100 |
| 33 | <100 | 34 | <100 | 35 | <100 | 36 | <100 |
| 37 | <100 | 38 | <100 | 39 | <100 | 40 | 100 < DC50 < 1000 |
| 41 | 1000 < DC50 < 5000 | 42 | <100 | 43 | <100 | 44 | <100 |
| 48 | <100 | 49 | <100 | 50 | <100 | 51 | <100 |
| 52 | <100 | 53 | <100 | 56 | <100 | 57 | <100 |
| 59 | <100 | 60 | <100 | 61 | <100 | 62 | <100 |
| 66 | <100 | 69 | <100 | 70 | <100 | 71 | <100 |
| 75 | <100 | 77 | <100 | 79 | <100 | 80 | <100 |
| 81 | <100 | 82 | <100 | 83 | <100 | 84 | <100 |
| 85 | <100 | 86 | <100 | 89 | <100 | 90 | <100 |
| 94 | <100 | 95 | <100 | 97 | <100 | 98 | <100 |
| 99 | <100 | 100 | <100 | 101 | <100 | 105 | 100 < DC50 < 1000 |
| 108 | 100 < DC50 < 1000 | 109 | <100 | 110 | <100 | 112 | <100 |
| 113 | <100 | 114 | 100 < DC50 < 1000 | 115 | <100 | 116 | <100 |
| 130 | <100 | 131 | <100 | 132 | <100 | 133 | <100 |
| 134 | <100 | 135 | <100 | 136 | <100 | 137 | <100 |

The above description is only for the purpose of illustrating the preferred example of the present invention, and is not intended to limit the scope of the present invention. Any modifications, equivalents and the like made without departing from the spirit and principle of the present invention should be included in the protection scope of the present invention.

The foregoing examples and methods described herein may vary based on the abilities, experience, and preferences of those skilled in the art.

The certain order in which the steps of the method are listed in the present invention does not place any limitation on the order of the steps of the method.

What is claimed is:

1. A compound of Formula A, or a pharmaceutically acceptable salt thereof, 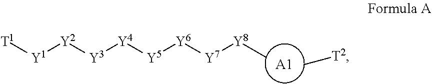
wherein:
T¹ is a glutarimide containing moiety;
T² is hydrogen or an optionally substituted fused 8-14 membered bicyclic or tricyclic heteroaryl having 1-6 ring heteroatoms each independently selected from N, S, and O, preferably, T² is not hydrogen;
Ring A1 is a 4-10 membered monocyclic or bicyclic carbocyclic or heterocyclic ring, which is optionally substituted with one or more substituents each independently halogen, OH, CN, oxo, NH₂, C₁₋₆ alkyl, C₂₋₆ alkenyl, C₂₋₆ alkynyl, C₁₋₆ heteroalkyl, or a 3-7 membered ring selected from C₃₋₇ carbocyclic, 3-7 membered heterocyclic, phenyl, or 5 or 6 membered heteroaryl, wherein the C₁₋₆ alkyl, C₂₋₆ alkenyl, C₂₋₆ alkynyl, C₁₋₆ heteroalkyl, or 3-7 membered ring is optionally substituted;
Y¹ to Y⁷ are defined according to (1)-(11) below:
Y¹ is NH,O,S,S(O),SO₂,C(G^{A})(G^{B}),C(O), or N(G^{C}); Y² is NH,O,S,S(O),SO₂,C(G^{A})(G^{B}),C(O), or N(G^{C}); Y³ is NH,O,S,S(O),SO₂,C(G^{A})(G^{B}),C(O), or N(G^{C}); Y⁴ is NH,O,S,S(O),SO₂,C(G^{A})(G^{B}),C(O), or N(G^{C}); Y⁵ is NH,O,S,S(O),SO₂,C(G^{A})(G^{B}),C(O), or N(G^{C}); Y⁶ is NH,O,S,S(O),SO₂,C(G^{A})(G^{B}),C(O), or N(G^{C}); Y⁷ is NH,O,S,S(O),SO₂,C(G^{A})(G^{B}),C(O), or N(G^{C}); (1)
wherein each of G^{A} and G^{B} at each occurrence is independently hydrogen, deuterium, halogen, OH, C₁₋₄ alkyl optionally substituted with halogen and/or OH, or C₁₋₄ heteroalkyl optionally substituted with halogen and/or OH, or two adjacent G^{A} are joined to form a double bond, or two adjacent C(G^{A})(G^{B}) represent a triple bond, or G^{A} and G^{B} together with the carbon atom they are both attached to are joined to form an optionally substituted 3-7 membered carbocyclic or heterocyclic ring; G^{C} at each occurrence is independently an optionally substituted group selected from C₁₋₄ alkyl, C₁₋₄ heteroalkyl, or a 3-8 membered ring;
provided that:
(ii) two adjacent groups of Y¹ to Y⁷ are not both selected from NH, O, S, or N(G^{C});
(iii) two adjacent groups of Y¹ to Y⁷ are not both selected from S(O), SO₂, or C(O);
(iv) at most three groups selected from Y¹ to Y⁷ can be S(O), SO₂, or C(O);
(v) at most two groups selected from Y¹ to Y⁷ can be C(G^{A})(G^{B}) wherein G^{A} and G^{B} together with the carbon atom they are both attached to are joined to form an optionally substituted 3-6 membered carbocyclic or heterocyclic ring; and
(vi) the combination of Y¹ to Y⁷ does not contain a bond selected from S—S(O), S—SO₂, and S—C(O);
(2) Y³, Y⁴, and Y⁵ together represent an optionally substituted 3-8 membered ring, and Y¹, Y², Y⁶, and Y⁷ are as defined in (1);
(3) Y⁴, Y⁵, and Y⁶ together represent an optionally substituted 3-8 membered ring, and Y¹, Y², Y³, and Y⁷ are as defined in (1);
(4) Y⁵, Y⁶, and Y⁷ together represent an optionally substituted 3-8 membered ring, and Y¹, Y², Y³, and Y⁴ are as defined in (1);
(5) Y³, Y⁴, Y⁵, and Y⁶ together represent an optionally substituted 3-10 membered ring, and Y¹, Y², and Y⁷ are as defined in (1);
(6) Y⁴, Y⁵, Y⁶, and Y⁷ together represent an optionally substituted 3-10 membered ring, and Y¹, Y², and Y³ are as defined in (1);
(7) Y², Y³, and Y⁴ together represent an optionally substituted 3-8 membered ring, and Y¹, Y⁵, Y⁶, and Y⁷ are as defined in (1);
(8) Y², Y³, Y⁴, and Y⁵ together represent an optionally substituted 3-10 membered ring, and Y¹, Y⁶, and Y⁷ are as defined in (1);
(9) Y², Y³, Y⁴, Y⁵, and Y⁶ together represent an optionally substituted 3-10 membered ring, and Y¹ and Y⁷ are as defined in (1);
(10) Y³, Y⁴, Y⁵, Y⁶, and Y⁷ together represent an optionally substituted 3-10 membered ring, preferably, 8-10 membered heterocyclic or heteroaryl ring, and Y¹ and Y² are as defined in (1);
(11) Y², Y³, Y⁴, Y⁵, Y⁶, and Y⁷ together represent an optionally substituted 3-10 membered ring, preferably, 8-10 membered heterocyclic or heteroaryl ring, and Y¹ is as defined in (1); and
Y⁸ is null, O, NH, C(O), an optionally substituted C₁₋₆ alkylene, or an optionally substituted C₁₋₆ heteroalkylene.

2. The compound of claim 1, or a pharmaceutically acceptable salt thereof, wherein T¹ is a moiety having a structure according to 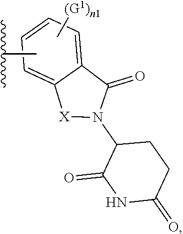
wherein:
X is CH₂ or C(═O),
n1 is an integer of 0-2, and
G¹ at each occurrence is independently halogen, CN, OH, NH₂, an optionally substituted C₁₋₆ alkyl, optionally substituted C₁₋₆ heteroalkyl, optionally substituted 3-8 membered carbocyclic or heterocyclic ring, optionally substituted phenyl, or optionally substituted heteroaryl.

3. The compound of claim 2, or a pharmaceutically acceptable salt thereof, characterized as having a structure according to Formula A1:

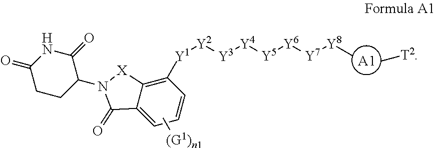

4. The compound of claim 2 or 3, or a pharmaceutically acceptable salt thereof, wherein n1 is 0.

5. The compound of any of claims 1-4, or a pharmaceutically acceptable salt thereof, wherein Y¹ is NH, O, or N(G^{C}), preferably, Y¹ is NH or O.

6. The compound of any of claims 1-5, or a pharmaceutically acceptable salt thereof, wherein Y² is C(G^{A})(G^{B}), preferably, each of G^{A} and G^{B} is independently hydrogen, deuterium, halogen, or C₁₋₄ alkyl (e.g., methyl), or G^{A} and G^{B} together with the carbon atom they are both attached to are joined to form a 3-6 membered carbocyclic or heterocyclic ring, which is optionally substituted with one or more substituents each independently selected from halogen, OH, and C₁₋₄ alkyl (e.g., methyl), for example, Y² is CH₂, CH(CH₃), C(CH₃)₂, 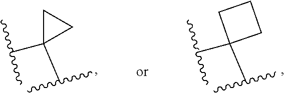 [SMILES: *C1(*)CC1 ; *C1(*)CCC1]
preferably, Y¹-Y² is —NHCH₂— or —OCH₂—.

7. The compound of any of claims 1-6, or a pharmaceutically acceptable salt thereof, wherein Y³, Y⁴, and Y⁵ together represent an optionally substituted 3-8 membered ring selected from 3-8 membered carbocyclic ring, 3-8 membered heterocyclic ring, phenyl ring, or 5- or 6-membered heteroaryl ring, preferably, Y³, Y⁴, and Y⁵ together represent an optionally substituted phenylene or an optionally substituted 5 or 6-membered heteroarylene.

8. The compound of any of claims 1-6, or a pharmaceutically acceptable salt thereof, wherein Y³, Y⁴, and Y⁵ together represent an optionally substituted phenylene or an optionally substituted 5 or 6-membered heteroarylene, wherein Y² and Y⁶ are not ortho to each other on the phenyl or heteroaryl ring, preferably, Y² and Y⁶ are meta to each other.

9. The compound of any of claims 1-6, or a pharmaceutically acceptable salt thereof, wherein Y³, Y⁴, and Y⁵ together represent a 1,3-phenylene, which is optionally substituted with one or more substituents each independently selected from halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

10. The compound of any of claims 1-6, or a pharmaceutically acceptable salt thereof, wherein Y³, Y⁴, and Y⁵ together represent a 5 or 6-membered heteroarylene, such as a 2,5-thiophenylene 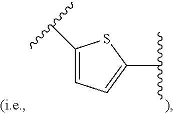 [SMILES: *c1ccc(*)s1]
2,5-furanylene, 2,4-pyridinylene, 2,6-pyridinylene, 3,5-pyridinylene, 2,4-pyrimidinylene, 2,6-pyrimidinylene, or 4,6-pyrimidinylene, etc., wherein the 5 or 6-membered heteroarylene is optionally substituted with one or more substituents each independently selected from halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

11. The compound of any of claims 1-6, or a pharmaceutically acceptable salt thereof, wherein Y³, Y⁴, and Y⁵ together represent a 3-6 membered carbocyclic or heterocyclic ring, which is optionally substituted with one or more substituents each independently oxo, halogen, OH, NH₂, C₁₋₄ alkyl optionally substituted with F, C₁₋₄ alkoxy optionally substituted with F, or a 3-4 membered carbocyclic or heterocyclic ring optionally substituted with F and/or methyl, for example, Y³, Y⁴, and Y⁵ together represent

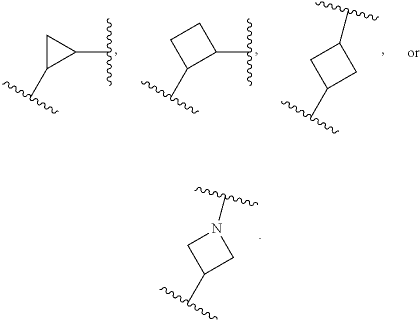 [SMILES: *C1CC1* ; *C1CCC1* ; *C1CC(*)C1 ; *C1CN(*)C1]

12. The compound of any of claims 1-6, or a pharmaceutically acceptable salt thereof, wherein Y³ is C(G^{A})(G^{B}), preferably, each of G^{A} and G^{B} is independently hydrogen, deuterium, halogen, or C₁₋₄ alkyl (e.g., methyl), or G^{A} and G^{B} together with the carbon atom they are both attached to are joined to form a 3-6 membered carbocyclic or heterocyclic ring, which is optionally substituted with one or more substituents each independently selected from halogen, OH, and C₁₋₄ alkyl (e.g., methyl), for example, Y³ is CH₂,

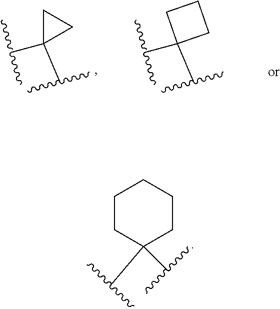 [SMILES: *C1(*)CC1 ; *C1(*)CCC1 ; *C1(*)CCCCC1]

13. The compound of any of claims 1-6 and 12, or a pharmaceutically acceptable salt thereof, wherein Y⁴ is O, NH, N(C₁₋₄ alkyl), or C(G^{A})(G^{B}), preferably, each of G^{A} and G^{B} is independently hydrogen, deuterium, halogen, or C₁₋₄ alkyl (e.g., methyl), or G^{A} and G^{B} together with the carbon atom they are both attached to are joined to form a 3-6 membered carbocyclic or heterocyclic ring, which is optionally substituted with one or more substituents each independently selected from halogen, OH, and C₁₋₄ alkyl (e.g., methyl), for example, Y⁴ is O, CH₂, CH(CH₃), C(CH₃)₂,

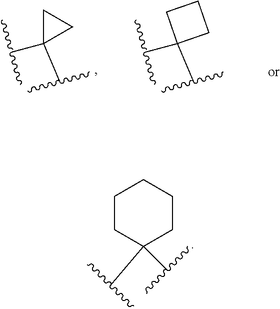 [SMILES: *C1(*)CC1 ; *C1(*)CCC1 ; *C1(*)CCCCC1]

14. The compound of any of claims 1-6 and 12-13, or a pharmaceutically acceptable salt thereof, wherein Y⁵ is O, NH, N(C₁₋₄ alkyl), or C(G^{A})(G^{B}), preferably, each of G^{A} and G^{B} is independently hydrogen, deuterium, halogen, or C₁₋₄ alkyl (e.g., methyl), or G^{A} and G^{B} together with the carbon atom they are both attached to are joined to form a 3-6 membered carbocyclic or heterocyclic ring, which is optionally substituted with one or more substituents each independently selected from halogen, OH, and C₁₋₄ alkyl (e.g., methyl), for example, Y⁵ is O or CH₂.

15. The compound of any of claims 1-6 and 12-13, or a pharmaceutically acceptable salt thereof, wherein Y⁵, Y⁶, and Y⁷ together represent an optionally substituted 3-8 membered ring selected from 3-8 membered carbocyclic ring, 3-8 membered heterocyclic ring, phenyl ring, or heteroaryl ring, for example, Y⁵, Y⁶, and Y⁷ together represent

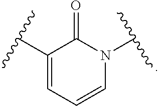 [SMILES: *c1cccn(*)c1=O]

16. The compound of any of claims 1-6 and 12-14, or a pharmaceutically acceptable salt thereof, wherein Y⁶ is O, C(O) or C(G^{A})(G^{B}), preferably, each of G^{A} and G^{B} is independently hydrogen, deuterium, halogen, or C₁₋₄ alkyl (e.g., methyl), for example, Y⁶ is O, C(O) or CH₂.

17. The compound of any of claims 1-6, 12-14, and 16, or a pharmaceutically acceptable salt thereof, wherein Y⁷ is O, C(O), NH, N(G^{C}), or C(G^{A})(G^{B}), preferably, each of G^{A} and G^{B} is independently hydrogen, deuterium, halogen, or C₁₋₄ alkyl (e.g., methyl), preferably, G^{C} is C₁₋₆ alkyl (e.g., methyl) or C₁₋₆ heteroalkyl, for example, Y⁷ is NH, O, CH₂, or N(CH₃).

18. The compound of any of claims 1-6 and 12-14, or a pharmaceutically acceptable salt thereof, wherein Y⁶-Y⁷ is —C(O)NH—, —C(O)—N(G^{C})-, —NHCH₂—, or —OCH₂—, wherein G^{C} is C₁₋₆ alkyl (e.g., methyl) or C₁₋₆ heteroalkyl, for example, Y⁶—Y⁷ is —C(O)NH—, —C(O)—N(CH₃)—, —NHCH₂—, or —OCH₂—.

19. The compound of any of claims 1-6 and 12, or a pharmaceutically acceptable salt thereof, wherein Y⁴, Y⁵, Y⁶, and Y⁷ together represent an optionally substituted 3-8 membered ring selected from 3-8 membered carbocyclic ring, 3-8 membered heterocyclic ring, phenyl ring, or heteroaryl ring, for example, Y⁴, Y⁵, Y⁶, and Y⁷ together represent

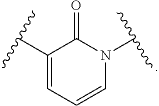 [SMILES: *c1cccn(*)c1=O]

20. The compound of any of claims 1-5, or a pharmaceutically acceptable salt thereof, wherein Y², Y³, Y⁴, Y⁵, Y⁶, and Y⁷ together represent an optionally substituted 8-10 membered heterocyclic or heteroaryl ring.

21. The compound of any of claims 1-6, or a pharmaceutically acceptable salt thereof, wherein Y³, Y⁴, Y⁵, Y⁶, and Y⁷ together represent an optionally substituted 8-10 membered heterocyclic or heteroaryl ring, preferably, a 6,5-fused or 6,6-fused heterocyclic or heteroaryl ring, having 1-5 ring heteroatoms each independently O, N, or S.

22. The compound of any of claims 1-6, or a pharmaceutically acceptable salt thereof, wherein Y³, Y⁴, Y⁵, Y⁶, and Y⁷ together represent an optionally substituted 6,5-fused or 6,6-fused heterocyclic ring, in which a phenyl ring or 6-membered heteroaryl is fused with a 5- or 6-membered heterocyclic ring having 1 or 2 ring heteroatoms each independently O, N, or S, preferably, 1 ring nitrogen atom, for example, Y³, Y⁴, Y⁵, Y⁶ and Y⁷ together represent

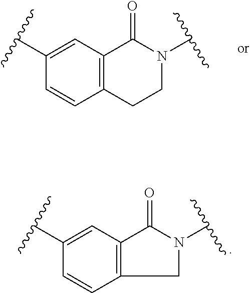 [SMILES: *c1ccc2c(c1)C(=O)N(*)CC2 ; *c1ccc2c(c1)C(=O)N(*)C2]

23. The compound of any of claims 1-6, or a pharmaceutically acceptable salt thereof, wherein Y³, Y⁴, Y⁵, Y⁶, and Y⁷ together represent an optionally substituted 6,5-fused or 6,6-fused heteroaryl ring having 1-5 ring heteroatoms each independently O, N, or S, for example, an optionally substituted benzimidazole, indazole, benzothiophene, etc., when substituted, the 6,5-fused or 6,6-fused heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

24. The compound of any of claims 1-23, or a pharmaceutically acceptable salt thereof, wherein Y⁸ is null.

25. The compound of any of claims 1-23, or a pharmaceutically acceptable salt thereof, wherein Y⁸ is O, NH, C(O), C₁₋₂ alkylene, or C₁₋₆ heteroalkylene having 1-3 heteroatoms independently selected from O, N, and S, wherein the S is optionally oxidized, and the C₁₋₆ heteroalkylene is optionally substituted with 1 or 2 oxo groups.

26. The compound of any of claims 1-25, or a pharmaceutically acceptable salt thereof, wherein Ring A1 is a 4-8 membered monocyclic carbocyclic or heterocyclic ring optionally substituted with one or more substituents each independently halogen, OH, CN, oxo, C₁₋₄ alkyl optionally substituted with F, or C₁₋₄ heteroalkyl optionally substituted with F.

27. The compound of any of claims 1-25, or a pharmaceutically acceptable salt thereof, wherein Ring A1 is a C₄₋₇cycloalkylene optionally substituted with one or more substituents each independently halogen, OH, CN, oxo, C₁₋₄ alkyl optionally substituted with F, or C₁₋₄ heteroalkyl optionally substituted with F.

28. The compound of any of claims 1-25, or a pharmaceutically acceptable salt thereof, wherein Ring A1 is cyclohexylene, preferably, a 1,4-trans-cyclohexylene,

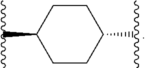 [SMILES: *[C@H]1CC[C@H](*)CC1]

29. The compound of any of claims 1-28, or a pharmaceutically acceptable salt thereof, wherein T2 is an optionally substituted 5,5-fused or 6,5-fused heteroaryl ring having 1-5 ring heteroatoms each independently O, N, or S, for example, an optionally substituted thiazolopyridine, imidazolopyridine, benzimidazole, pyrazolopyridine, oxazolopyridine, benzoxazole, indole, benzothiophene, benzothiazole, thienopyridine, thienopyrimidine, thienothiophene, etc., when substituted, the 5,5-fused or 6,5-fused heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl.

30. The compound of any of claims 1-28, or a pharmaceutically acceptable salt thereof, wherein T² is an optionally substituted 6,5-fused heteroaryl ring having 1-5 ring heteroatoms each independently O, N, or S, wherein one of the fused rings is a furan, thiophene, pyrrole, pyrazole, imidazole, oxazole, thiazole, isoxazole, or isothiazole, which is fused with a benzene, pyridine, pyrimidine, pyridazine, or pyrazine, wherein when substituted, the 6,5-fused heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl, preferably, T² connects to Ring A1 through a ring atom on the 5-membered ring, for example, T is an optionally substituted heteroaryl selected from:

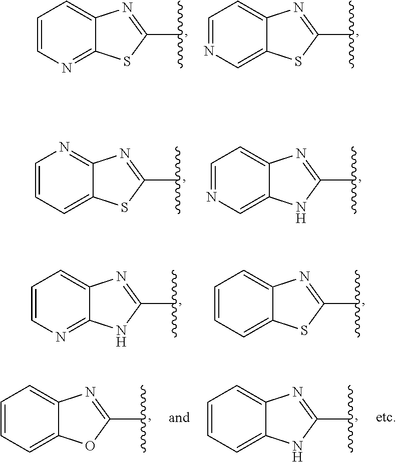

31. The compound of any of claims 1-28, or a pharmaceutically acceptable salt thereof, wherein T² is an optionally substituted 5,6,5-fused, 6,5,5-fused, or 5,6,6-fused tricyclic heteroaryl ring having 1-5 ring heteroatoms each independently O, N, or S, wherein each 5-membered ring of the fused rings is independently a furan, thiophene, pyrrole, pyrazole, imidazole, oxazole, thiazole, isoxazole, or isothiazole, and each 6-membered ring of the fused rings is independently benzene, pyridine, pyrimidine, pyridazine, or pyrazine, wherein when substituted, the 5,6,5-fused, 6,5,5-fused, or 5,6,6-fused tricyclic heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG^{Z}G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl, for example, T is an optionally substituted heteroaryl selected from:

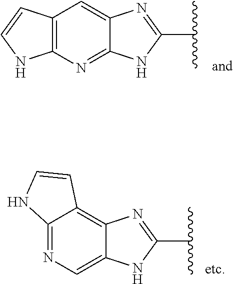 [SMILES: *c1nc2cc3cc[nH]c3nc2[nH]1 ; *c1nc2c(cnc3[nH]ccc32)[nH]1]

32. A compound of Formula B, or a pharmaceutically acceptable salt thereof, 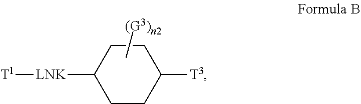
wherein:
T¹ is a glutarimide containing moiety;
T³ is an optionally substituted fused 8-14 membered bicyclic or tricyclic heteroaryl having 1-6 ring heteroatoms each independently selected from N, S, and O;
n2 is an integer of 0-2, and G³ at each occurrence is independently halogen, OH, CN, oxo, C₁₋₄ alkyl optionally substituted with F, or C₁₋₄ heteroalkyl optionally substituted with F;
preferably, n2 is 0; and
LNK is a linker that connects T to the cyclohexyl ring, wherein the linker is a chain, ring, or a ring-chain structure, wherein the smallest number of chain or ring forming atoms of the linker is at least 4 (e.g., 4, 5, 6, 7, 8, or 9), wherein each of the chain or ring forming atoms is independently selected from C, N, O, and S, wherein the smallest number is the least number of atoms of the linker needed to reach from T¹ to the cyclohexyl ring, starting from the atom that is bonded to T¹ and ending with the atom that is bonded to the cyclohexyl ring.

33. The compound of claim 32, or a pharmaceutically acceptable salt thereof, characterized as having a structure according to Formula B1:

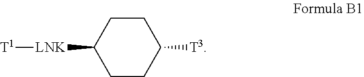

34. The compound of claim 32 or 33, or a pharmaceutically acceptable salt thereof, wherein T³ is an optionally substituted 6,5-fused heteroaryl ring having 1-5 ring heteroatoms each independently O, N, or S, wherein one of the fused rings is a furan, thiophene, pyrrole, pyrazole, imidazole, oxazole, thiazole, isoxazole, or isothiazole, which is fused with a benzene, pyridine, pyrimidine, pyridazine, or pyrazine, wherein when substituted, the 6,5-fused heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl, preferably, T³ connects to the cyclohexyl ring through a ring atom on the 5-membered ring, for example, T³ is an optionally substituted heteroaryl selected from:

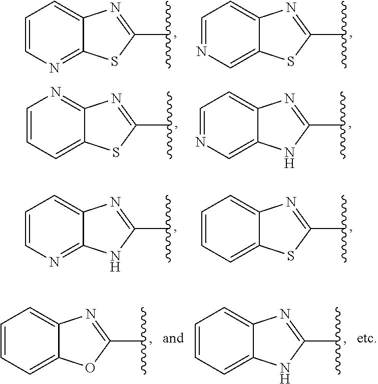

35. The compound of claim 32 or 33, or a pharmaceutically acceptable salt thereof, wherein T³ is an optionally substituted 5,6,5-fused, 6,5,5-fused, or 5,6,6-fused tricyclic heteroaryl ring having 1-5 ring heteroatoms each independently O, N, or S, wherein each 5-membered ring of the fused rings is independently a furane, thiophene, pyrrole, pyrazole, imidazole, oxazole, thiazole, isoxazole, or isothiazole, and each 6-membered ring of the fused rings is independently benzene, pyridine, pyrimidine, pyridazine, or pyrazine, wherein when substituted, the 5,6,5-fused, 6,5,5-fused, or 5,6,6-fused tricyclic heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl, preferably, T³ connects to the cyclohexyl ring through a ring atom on a 5-membered ring, for example, T³ is an optionally substituted heteroaryl selected from:

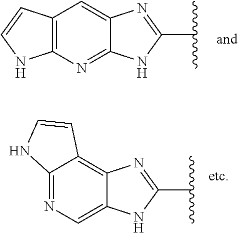 [SMILES: *c1nc2cc3cc[nH]c3nc2[nH]1 ; *c1nc2c(cnc3[nH]ccc32)[nH]1]

36. The compound of any of claims 32-35, or a pharmaceutically acceptable salt thereof, wherein T¹ is a moiety having a structure according to 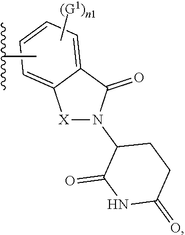
wherein:
X is CH₂ or C(═O),
n1 is an integer of 0-2, and
G¹ at each occurrence is independently halogen, CN, OH, NH₂, an optionally substituted C₁₋₆ alkyl, optionally substituted C₁₋₆ heteroalkyl, optionally substituted 3-8 membered carbocyclic or heterocyclic ring, optionally substituted phenyl, or optionally substituted heteroaryl.

37. The compound of claim 36, or a pharmaceutically acceptable salt thereof, characterized as having a structure according to Formula B2:

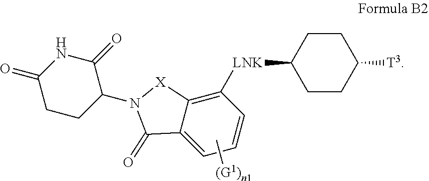

38. The compound of claim 36 or 37, or a pharmaceutically acceptable salt thereof, wherein n1 is 0.

39. The compound of any of claims 32-38, or a pharmaceutically acceptable salt thereof, wherein LNK is any linker defined herein, for example, a linker according to 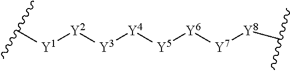
as defined in any of claims 5-25, or any linker as defined herein for L in connection with Formula (I).

40. A compound of Formula C, or a pharmaceutically acceptable salt thereof, 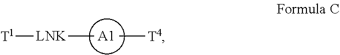
wherein:
T¹ is a glutarimide containing moiety;
LNK is a linker that connects T¹ to Ring A1, wherein the linker is a chain, ring, or a ring-chain structure, wherein the smallest number of chain or ring forming atoms of the linker is at least 4 (e.g., 4, 5, 6, 7, 8, or 9), wherein each of the chain or ring forming atoms is independently selected from C, N, O, and S, wherein the smallest number is the least number of atoms of the linker needed to reach from T¹ to Ring A1, starting from the atom that is bonded to T¹ and ending with the atom that is bonded to Ring A1;
Ring A1 is an optionally substituted 4-10 membered monocyclic or bicyclic carbocyclic or heterocyclic ring; and
T⁴ is an optionally substituted 5,5-fused or 6,5-fused heteroaryl ring having 1-5 ring heteroatoms each independently O, N, or S, or an optionally substituted 5,6,5-fused, 6,5,5-fused, or 5,6,6-fused tricyclic heteroaryl ring having 1-6 ring heteroatoms each independently O, N, or S, preferably, a ring atom of a 5-membered ring of T⁴ is bonded to Ring A1.

41. The compound of claim 40, or a pharmaceutically acceptable salt thereof, wherein T⁴ is an optionally substituted 6,5-fused heteroaryl ring having 1-5 ring heteroatoms each independently O, N, or S, wherein one of the fused rings is a furan, thiophene, pyrrole, pyrazole, imidazole, oxazole, thiazole, isoxazole, or isothiazole, which is fused with a benzene, pyridine, pyrimidine, pyridazine, or pyrazine, wherein when substituted, the 6,5-fused heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl, for example, T⁴ is an optionally substituted heteroaryl selected from:

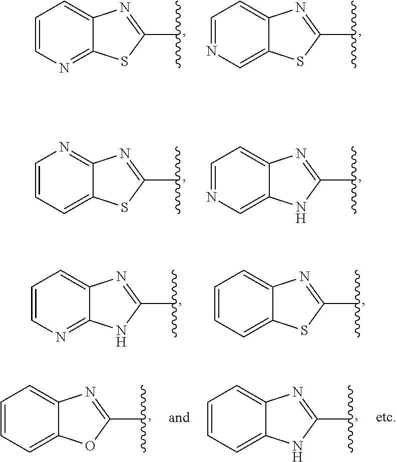

42. The compound of claim 40, or a pharmaceutically acceptable salt thereof, wherein T⁴ is an optionally substituted 5,6,5-fused, 6,5,5-fused, or 5,6,6-fused tricyclic heteroaryl ring having 1-5 ring heteroatoms each independently O, N, or S, wherein each 5-membered ring of the fused rings is independently a furane, thiophene, pyrrole, pyrazole, imidazole, oxazole, thiazole, isoxazole, or isothiazole, and each 6-membered ring of the fused rings is independently benzene, pyridine, pyrimidine, pyridazine, or pyrazine, wherein when substituted, the 5,6,5-fused, 6,5,5-fused, or 5,6,6-fused tricyclic heteroaryl ring is preferably substituted with 1-3 substituents each independently halogen, CN, G², OH, NH₂, O-G², NHG², NG²G², COOH, CONH₂, C(O)O-G², C(O)NHG², or C(O)NG²G², wherein G² at each occurrence is independently an optionally substituted C₁₋₄ alkyl, optionally substituted C₂₋₄ alkenyl, optionally substituted C₂₋₄ alkynyl, optionally substituted C₁₋₄ heteroalkyl, or optionally substituted 3-7 membered ring (preferably 3-7 membered carbocyclic or heterocyclic ring), preferably, when substituted, the optionally substituted group is substituted with one or more substituents each independently oxo (as applicable), halogen, OH, CN, NH₂, optionally substituted C₁₋₄ alkyl, optionally substituted C₁₋₄ heteroalkyl, or an optionally substituted 3-4 membered carbocyclic or heterocyclic ring; more preferably, G² at each occurrence is independently C₁₋₄ alkyl optionally substituted with 1-3 G^{S1}, C₁₋₄ heteroalkyl optionally substituted with 1-3 G^{S2}, or a 3-7 membered ring (preferably 3-7 membered carbocyclic or heterocyclic ring) optionally substituted with 1-3 G^{S2}, wherein G^{S1} at each occurrence is independently halogen, OH, C₁₋₄ heteroalkyl optionally substituted with F, or a 3-4 membered carbocyclic or heterocyclic ring optionally substituted with F and/or methyl; and G^{S2} at each occurrence is independently oxo (as applicable), halogen, OH, NH₂, C₁₋₄ alkyl optionally substituted with F, C₁₋₄ heteroalkyl optionally substituted with F, or a 3-4 membered carbocyclic or heterocyclic ring optionally substituted with F and/or methyl, for example, T⁴ is an optionally substituted heteroaryl selected from:

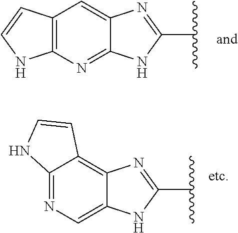 [SMILES: *c1nc2cc3cc[nH]c3nc2[nH]1 ; *c1nc2c(cnc3[nH]ccc32)[nH]1]

43. The compound of any of claims 40-42, or a pharmaceutically acceptable salt thereof, wherein T¹ is a moiety having a structure according to 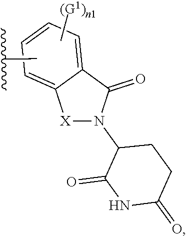
wherein:
X is CH₂ or C(═O),
n1 is an integer of 0-2, and
G¹ at each occurrence is independently halogen, CN, OH, NH₂, an optionally substituted C₁₋₆ alkyl, optionally substituted C₁₋₆ heteroalkyl, optionally substituted 3-8 membered carbocyclic or heterocyclic ring, optionally substituted phenyl, or optionally substituted heteroaryl.

44. The compound of claim 43, or a pharmaceutically acceptable salt thereof, characterized as having a structure according to Formula C1:

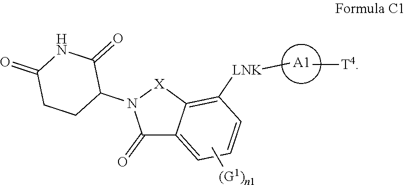

45. The compound of claim 43 or 44, or a pharmaceutically acceptable salt thereof, wherein n1 is 0.

46. The compound of any of claims 40-45, or a pharmaceutically acceptable salt thereof, wherein LNK is any linker defined herein, for example, a linker according to 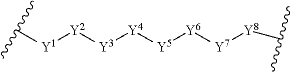
as defined in any of claims 5-25, or any linker as defined herein for L in connection with Formula (I).

47. The compound of any of claims 40-46, or a pharmaceutically acceptable salt thereof, wherein Ring A1 is an optionally substituted 4-8 membered monocyclic carbocyclic or heterocyclic ring.

48. The compound of any of claims 40-46, or a pharmaceutically acceptable salt thereof, wherein Ring A1 is an optionally substituted C₄₋₇cycloalkylene.

49. The compound of any of claims 40-46, or a pharmaceutically acceptable salt thereof, wherein Ring A1 is cyclohexylene, preferably, a 1,4-trans-cyclohexylene,

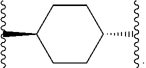 [SMILES: *[C@H]1CC[C@H](*)CC1]

50. A compound selected from any of Examples 1-150, or a pharmaceutically acceptable salt thereof.

51. A pharmaceutical composition comprising the compound of any of claims 1-50 or a pharmaceutically acceptable salt thereof and a pharmaceutically acceptable excipient.

52. A method of inducing degradation of a protein in a cell, the method comprising contacting the cell with the compound of any of claims 1-50 or a pharmaceutically acceptable salt thereof.

53. The method of claim 52, wherein the protein is GSPT1.

54. The method of claim 52 or 53, wherein the cell is a cancer cell.

55. A method of treating cancer in a subject in need thereof, the method comprising administering to the subject a therapeutically effective amount of the compound of any of claims 1-50 or a pharmaceutically acceptable salt thereof or the pharmaceutical composition of claim 51.

56. The method of claim 55, wherein the cancer is associated with GSPT1 activity.

57. A compound, or a pharmaceutically acceptable salt, a stereoisomer, an ester, a prodrug, a solvate and a deuterated compound thereof, the compound having the following structure: 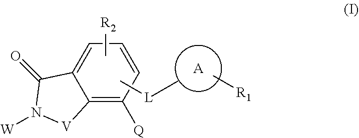
wherein,
W is selected from: C₀₋₁₀ alkyl, C₃₋₆ cycloalkyl, heterocycloalkyl, and heteroaryl;
R₁ is one or more independent substituents of ring A and is selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), and 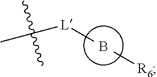
L′ is selected from: single bond, C₁₋₁₀ alkylene, —O—, —O—C₁₋₁₀ alkylene-, —S—, —S—C₁₋₁₀ alkylene-, —SO—, —SO—C₁₋₁₀ alkylene-, —SO₂—, —SO₂—C₁₋₁₀ alkylene-, —N(C₀₋₁₀ alkyl)-, —N—(C₀₋₁₀ alkyl) alkylene-, —CO—, —CO—C₁₋₁₀ alkylene-, —CONH—, and —CONH—C₁₋₁₀ alkylene-; R₆ is one or more independent substituents on ring B and is selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl;
R₂ is one or more independent substituents of a benzene ring and is selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, aryl, heterocycloalkyl, and heteroaryl;
V is selected from: O, S, 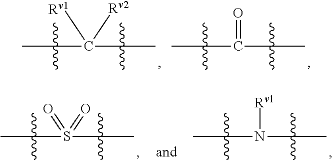
wherein R^{v1}, R^{v2} is independently selected from: C₀₋₁₀ alkyl, C₃₋₆ cycloalkyl, and heterocycloalkyl;
Q is H or a single bond, or Q and V, together with carbon atoms therebetween, form aryl or heteroaryl;
L has the following structure: 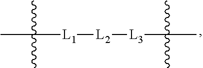
wherein,
L₁ is selected from: —NR^{L1}—(C₀₋₁₀ alkylene)-, —O—(C₀₋₁₀ alkylene)-, —S—(C₀₋₁₀ alkylene)-, —NR^{L1}CO—(C₀₋₁₀ alkylene)-, —CONR^{L1}—(C₀₋₁₀ alkylene)-, —CO—(C₀₋₁₀ alkylene)-, —NR^{L1}CONH—(C₀₋₁₀ alkylene)-, —(C₁₋₁₀ alkylene)-, —SO₂—(C₀₋₁₀ alkylene)-, —SO—(C₀₋₁₀ alkylene)-, 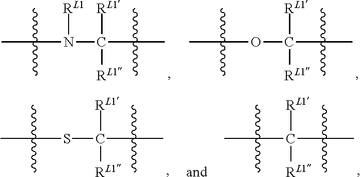
wherein R^{L1} is selected from: H, —OH, C₁₋₆ alkyl, C₃₋₆ cycloalkyl, heterocycloalkyl, wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, C₃₋₆ cycloalkyl, and heterocycloalkyl; R^{L1′} and R^{L1″} are independently selected from: halogen, C₁₋₆ alkyl, C₃₋₆ cycloalkyl, heterocycloalkyl, wherein one or more H in the C₁₋₆alkyl, C₃₋₆ cycloalkyl or heterocycloalkyl may be substituted with a substituent selected from: halogen (particularly F), —OH, —NH₂, alkoxy, alkylamino, C₃₋₆ cycloalkyl, and heterocycloalkyl; or R^{L1′} and R^{L″}, together with carbon atoms linked thereto, form cycloalkyl or heterocyclyl;
L₃ is selected from: —(C₀₋₁₀ alkylene)-, —(C₀₋₁₀ alkylene)-NR^{L3}—, —(C₀₋₁₀ alkylene)-O—, —(C₀₋₁₀ alkylene)-S—, —(C₀₋₁₀ alkylene)-NR^{L3}CO—, —(C₀₋₁₀ alkylene)-CONR^{L3}—, —(C₀₋₁₀ alkylene)-CO—, —(C₀₋₁₀ alkylene)-NHCONR^{L3}—, —(C₀₋₁₀ alkylene)-SO₂—, —(C₀₋₁₀ alkylene)-SO—, 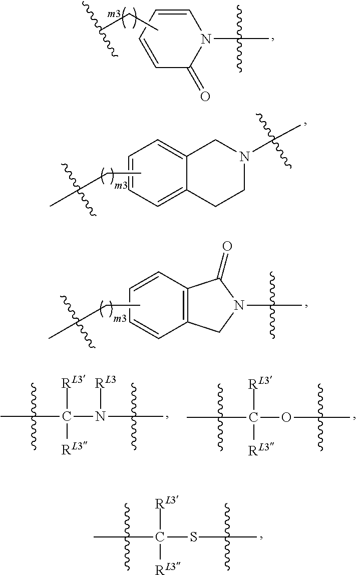
—NR^{L3}—(C₀₋₁₀ alkylene)-, —O—(C₀₋₁₀ alkylene)-, —S—(C₀₋₁₀ alkylene)-, —NR^{L3}CO—(C₀₋₁₀ alkylene)-, —CONR^{L3}—(C₀₋₁₀ alkylene)-, —CO—(C₀₋₁₀ alkylene)-, —NHCONR^{L3}—(C₀₋₁₀ alkylene)-, —SO₂—(C₀₋₁₀ alkylene)-, —SO—(C₀₋₁₀ alkylene)-, 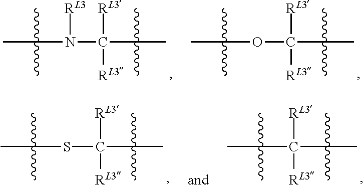
wherein R^{L3} is selected from: H, —OH, C₁₋₆ alkyl, C₃₋₆ cycloalkyl, and heterocycloalkyl, wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, C₃₋₆ cycloalkyl, and heterocycloalkyl; R^{L3′} and R^{L3″} are independently selected from: halogen, C₁₋₆ alkyl, C₃₋₆ cycloalkyl, and heterocycloalkyl, wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, C₃₋₆ cycloalkyl, heterocycloalkyl; or R^{L3′} and R^{L3″}, together with carbon atoms linked thereto, form cycloalkyl or heterocyclyl;
L₂ is a covalent bond, or a divalent, saturated or unsaturated, linear or branched C1-50 hydrocarbon chain, wherein 0-6 methylene units are independently substituted with: -Cy-, —O—, —NR^{L2}—, —S—, —OC(O)—, —C(O)O—, —C(O)—, —S(O)—, —S(O)₂—, —NR^{L2}S(O)₂—, —S(O)₂—NR^{L2}—, —NR^{L2}—C(O)—C(O)NR^{L2}—, —OC(O)NR^{L2}—, —NR^{L2}—C(O)O—, 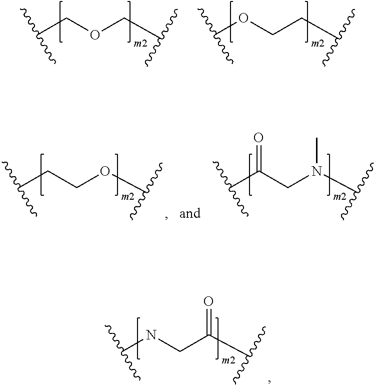
wherein m2 is an integer selected from 0-10, and each -Cy- is independently selected from the following optionally substituted divalent rings: arylene, cycloalkylene, and heterocyclylene; R^{L2} is selected from: H, —OH, C₁₋₆ alkyl, C₃₋₆ cycloalkyl, and heterocycloalkyl, wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, C₃₋₆ cycloalkyl, and heterocycloalkyl.

58. The compound according to claim 57, wherein W is 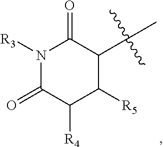
wherein R₃ is selected from: C₀₋₁₀ alkyl, C₃₋₆ cycloalkyl, heterocycloalkyl, and heteroaryl;
R₄ and R₅ are independently selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl;
preferably, W is

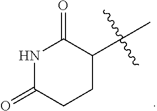 [SMILES: *C1CCC(=O)NC1=O]

59. The compound according to claim 57 or 58, wherein V is selected from: —CH₂—, 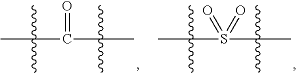
and —NH—; preferably, V is

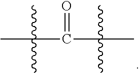 [SMILES: *C(*)=O]

60. The compound according to any one of claims 57 to 59, having the following structure: 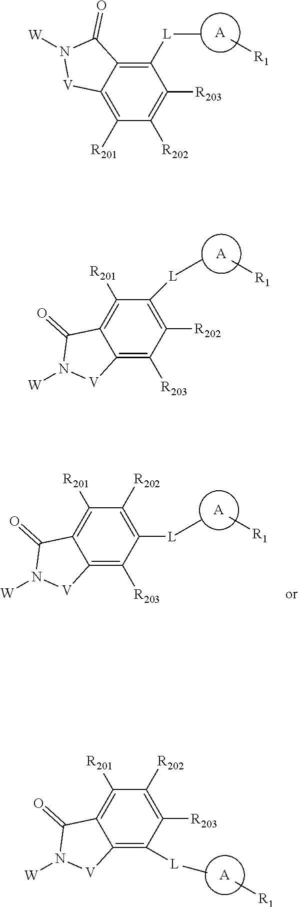
wherein, R₂₀₁, R₂₀₂ and R₂₀₃ are independently selected from: cycloalkyl, aryl, heterocycloalkyl, and heteroaryl; or R₂₀₁, R₂₀₂ and R₂₀₃ are independently selected from: halogen, —CN, —NO₂, —CF₃, —OCF₃, —H, C₁₋₆ alkyl, —OH, —NH₂, and —N(C₁₋₃ alkyl)(C₁₋₃alkyl);
preferably, R₂₀₁, R₂₀₂ and R₂₀₃ are all —H.

61. The compound according to claim 57 or 58, having the following structures: 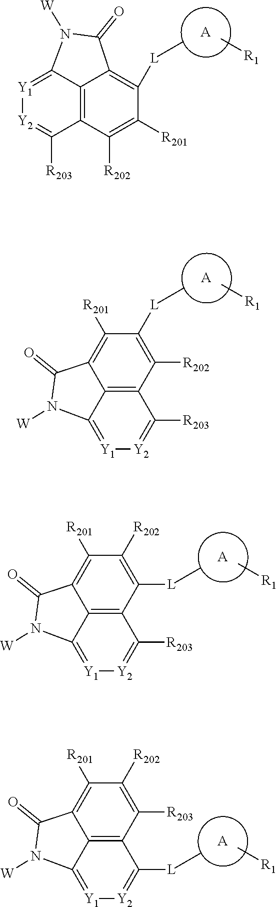
wherein Y₁ and Y₂ are independently selected from: CH and N; R₂₀₁, R₂₀₂ and R₂₀₃ are independently selected from: cycloalkyl, aryl, heterocycloalkyl, and heteroaryl; or R₂₀₁, R₂₀₂ and R₂₀₃ are independently selected from: halogen, —CN, —NO₂, —CF₃, —OCF₃, —H, C₁₋₆ alkyl, —OH, —NH₂, and —N(C₁₋₃ alkyl)(C₁₋₃alkyl);
preferably, Y₁ and Y₂ are both CH; or Y₁ is CH, Y₂ is N; or Y₁ is N, Y₂ is CH; or Y₁ and Y₂ are both N;
preferably, R₂₀₁, R₂₀₂ and R₂₀₃ are all —H.

62. The compound according to any one of claims 57 to 61, wherein ring A is a benzene ring, an 8- to 10-membered bicyclic aromatic ring, a 3- to 7-membered monocyclic saturated aliphatic ring, a 4- to 7-membered saturated spiro aliphatic ring, an 8- to 10-membered saturated fused aliphatic ring, an 8-10-membered saturated bridged aliphatic ring, a 4- to 7-membered monocyclic saturated heterocyclic ring, a 4- to 7-membered saturated spiro heterocyclic ring, an 8- to 10-membered saturated fused heterocyclic ring, or an 8- to 10-membered saturated bridged heterocyclic ring;
preferably, moiety 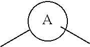
is selected from: 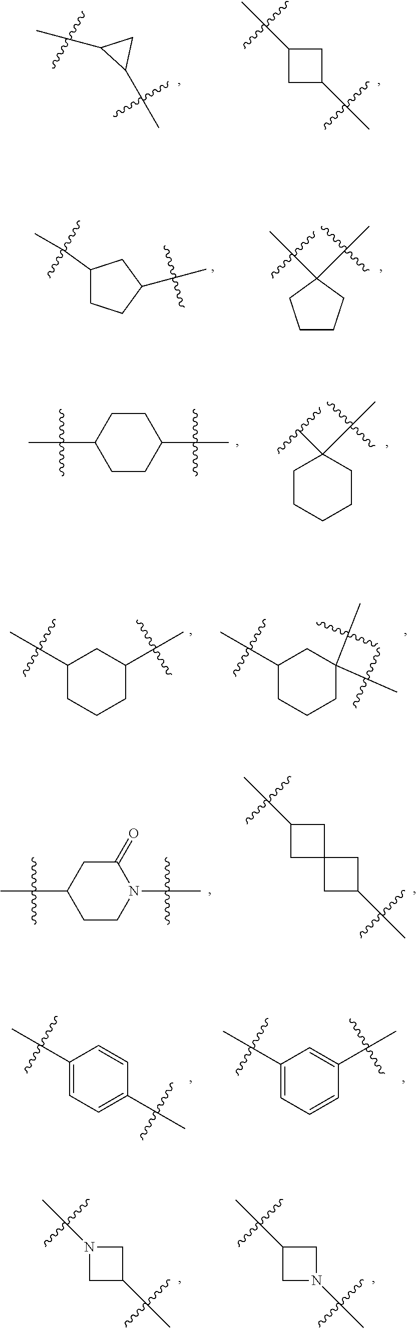 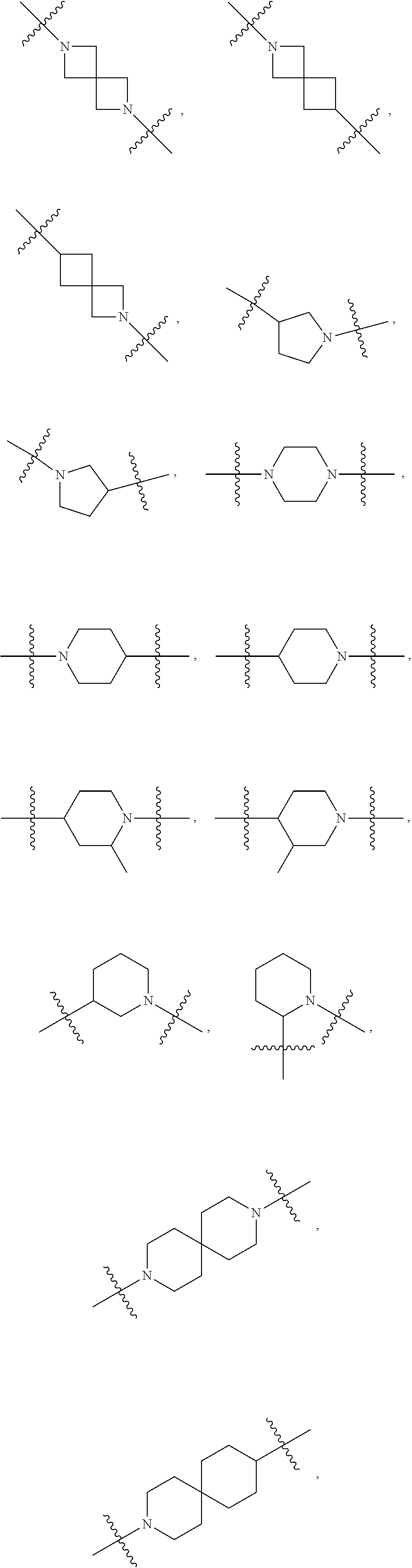 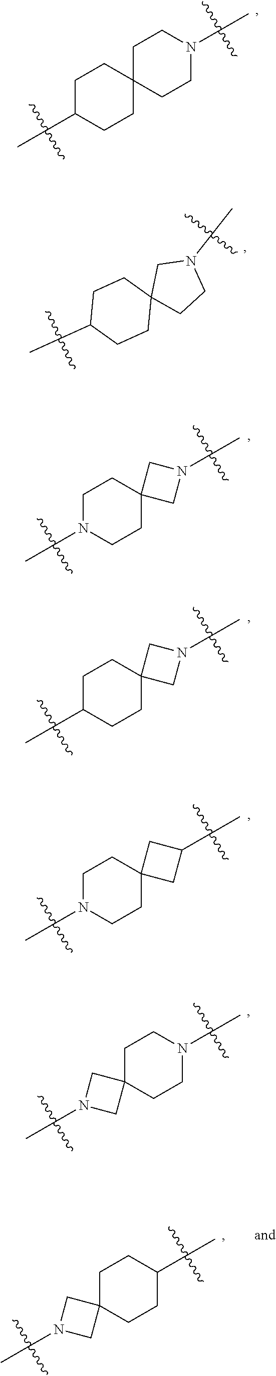 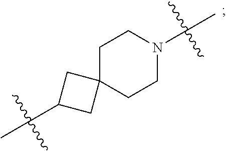 [SMILES: *C1CC2(CCN(*)CC2)C1]
more preferably, moiety is, 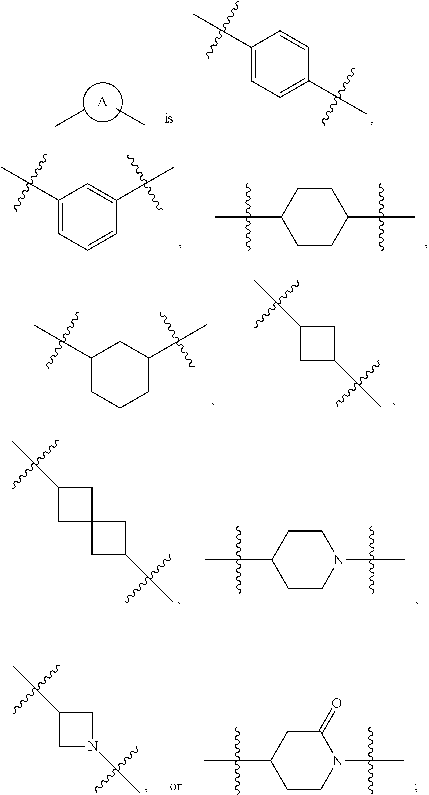
further preferably, moiety

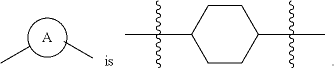

63. The compound according to any one of claims 57 to 62, wherein ring B is a benzene ring, a bicyclic aromatic ring, a tricyclic aromatic ring, a monocyclic heteroaromatic ring, a bicyclic heteroaromatic ring, or a tricyclic heteroaromatic ring.

64. The compound according to any one of claims 57 to 63, wherein moiety 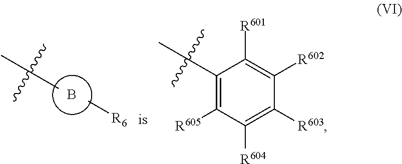
wherein R⁶⁰¹, R⁶⁰², R⁶⁰³, R⁶⁰⁴ and R⁶⁰⁵ are independently selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ to alkyl), —CON(C₀₋₁₀ alkyl)(C₀₋₁₀ alkyl), —CO(C₀₋₁₀ alkyl), cycloalkyl, heterocycloalkyl, and heteroaryl, wherein H at the carbon atoms may be substituted with one or more groups selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl;
preferably, R⁶⁰¹, R⁶⁰², R⁶⁰³, R⁶⁰⁴ and R⁶⁰⁵ are independently selected from: —H, halogen, —OH, C₁₋₆ alkyl, and —O(C₁₋₆ alkyl).

65. The compound according to any one of claims 57 to 63, wherein moiety 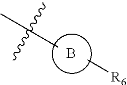
has the following structure: 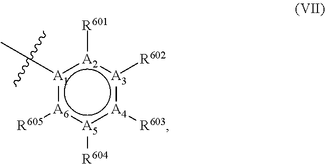
wherein A₁, A₂, A₃, A₄, A₅ and A₆ are independently selected from C and N, and at least one of A₁, A₂, A₃, A₄, A₅ and A₆ is N; when any one of A₂, A₃, A₄, A₅ and A₆ is N, R⁶⁰¹, R⁶⁰², R⁶⁰³, R⁶⁰⁴ and R⁶⁰⁵ corresponding thereto is absent; R⁶⁰¹, R⁶⁰², R⁶⁰³, R⁶⁰⁴ and R⁶⁰⁵ are independently selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl, wherein H at the carbon atoms may be substituted with one or more groups selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl;
preferably, moiety 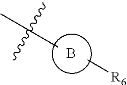
is selected from the following structures: 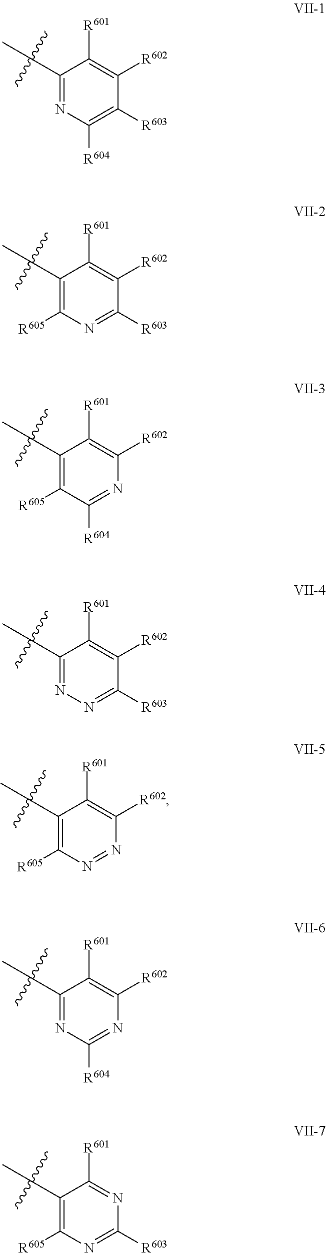 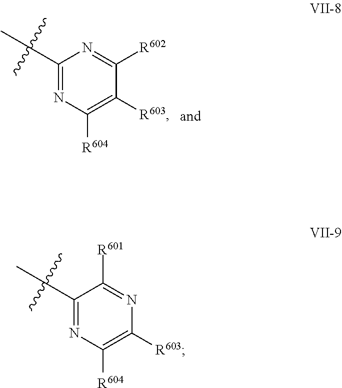
preferably, in formula VII, R⁶⁰¹, R⁶⁰², R⁶⁰³, R⁶⁰⁴ and R⁶⁰⁵ are independently selected from —H, -D, —F, —Cl, —Br, —I, —CF₃, —CHF₂, —CH₂F, —CH₂CF₃, —CN, —NO₂, —CH₃,

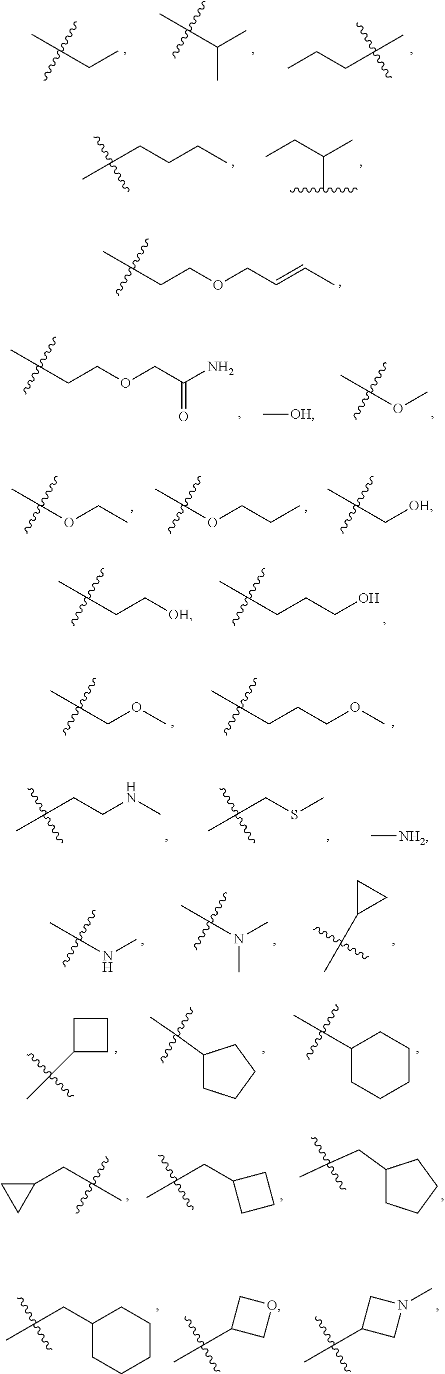

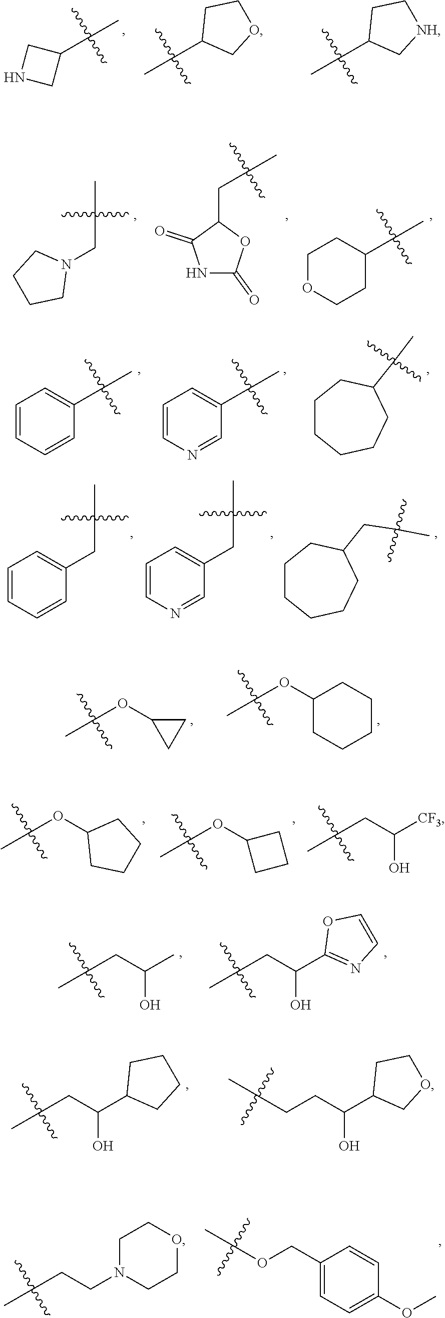

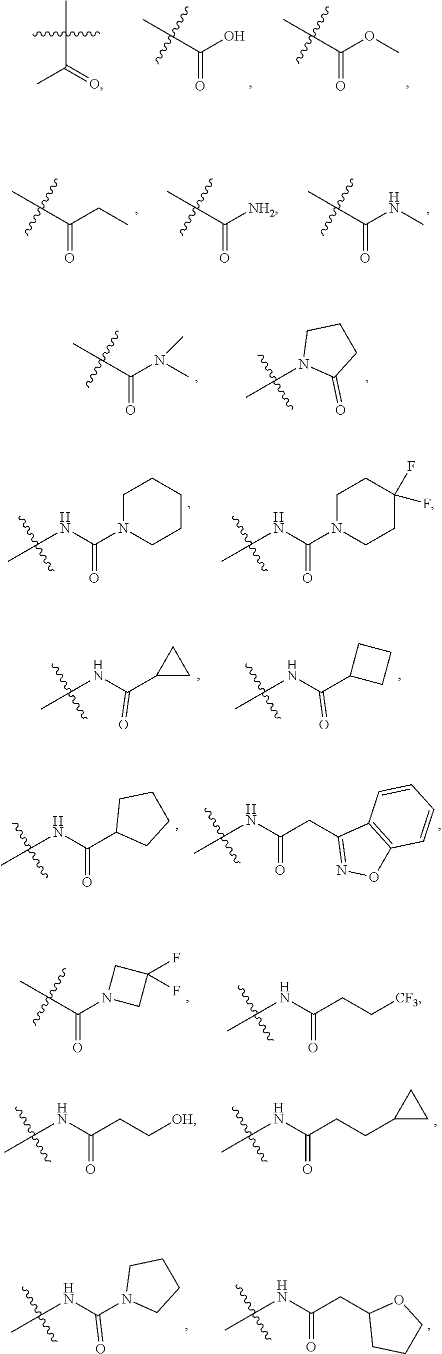

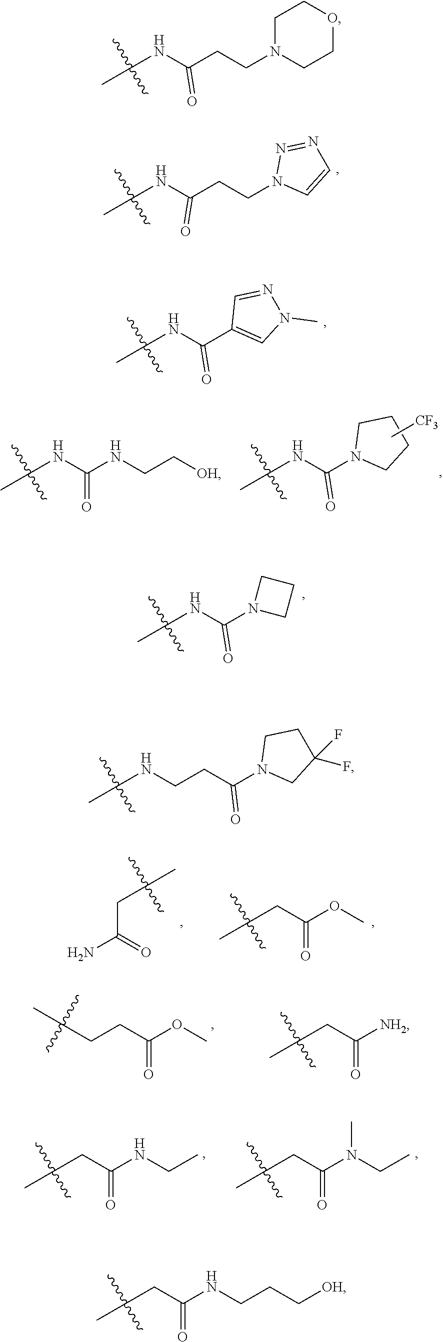

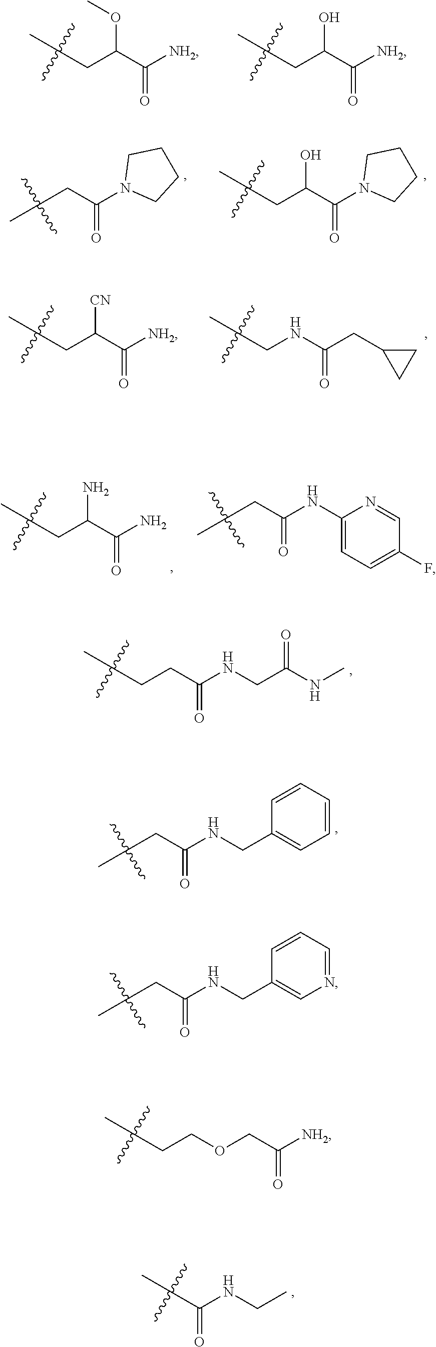

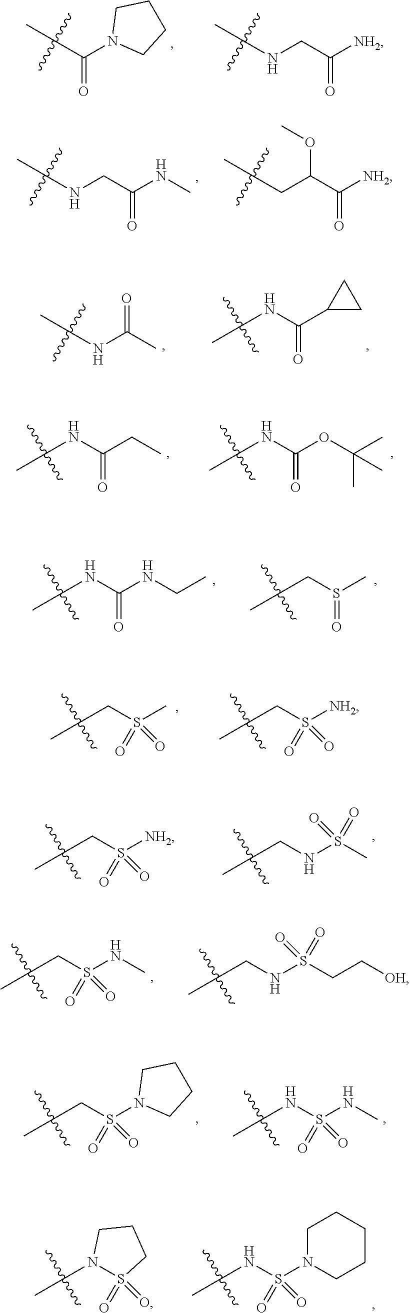

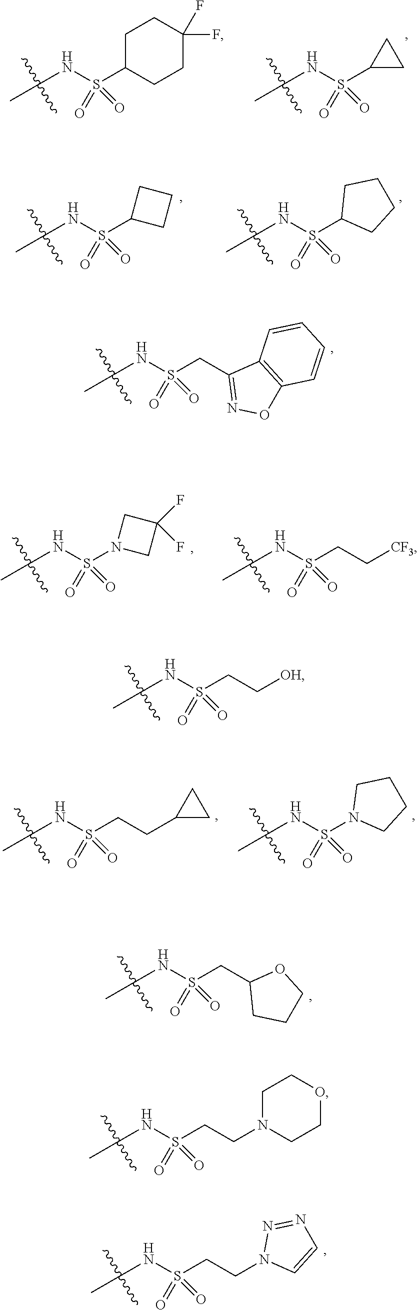

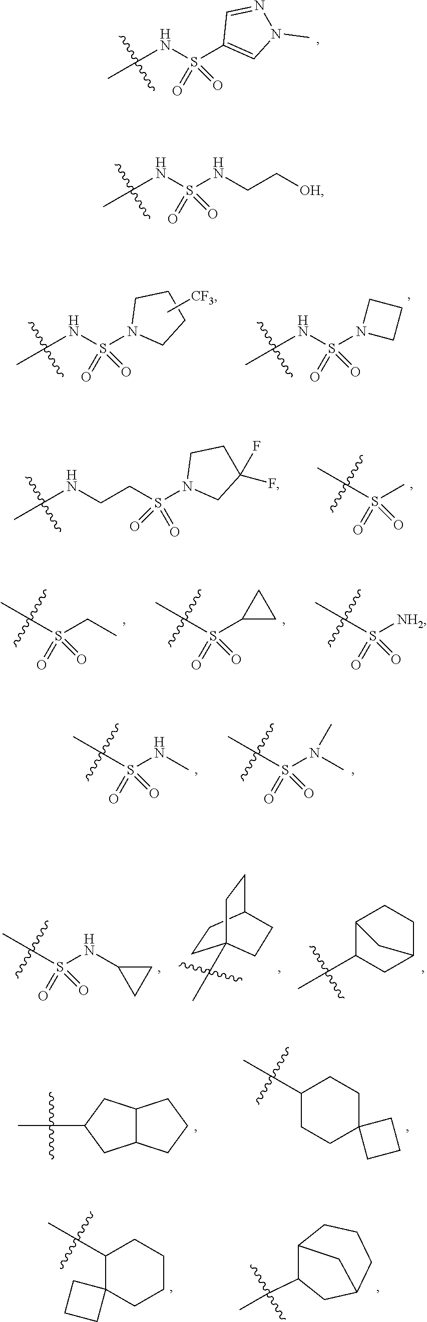

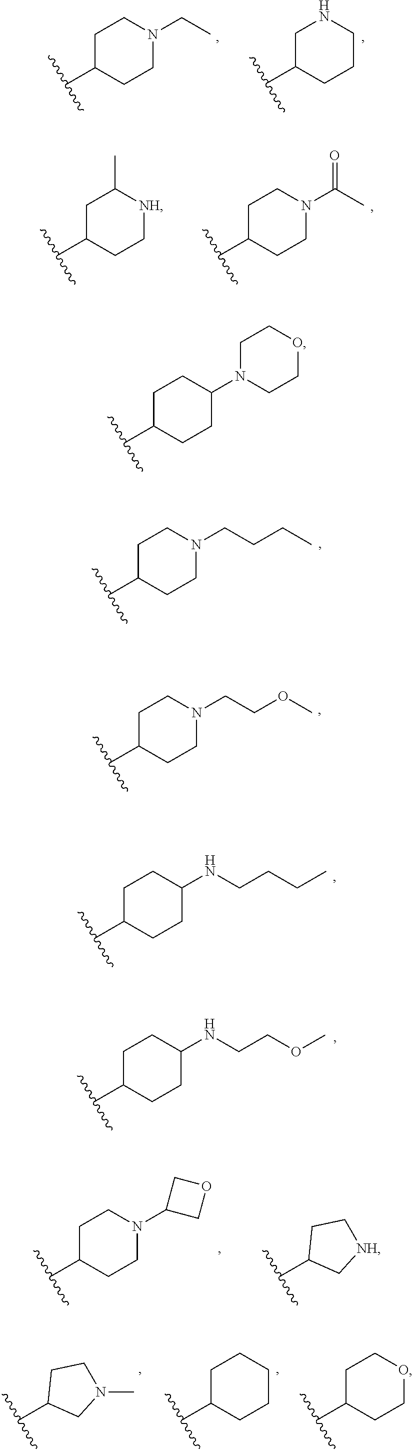

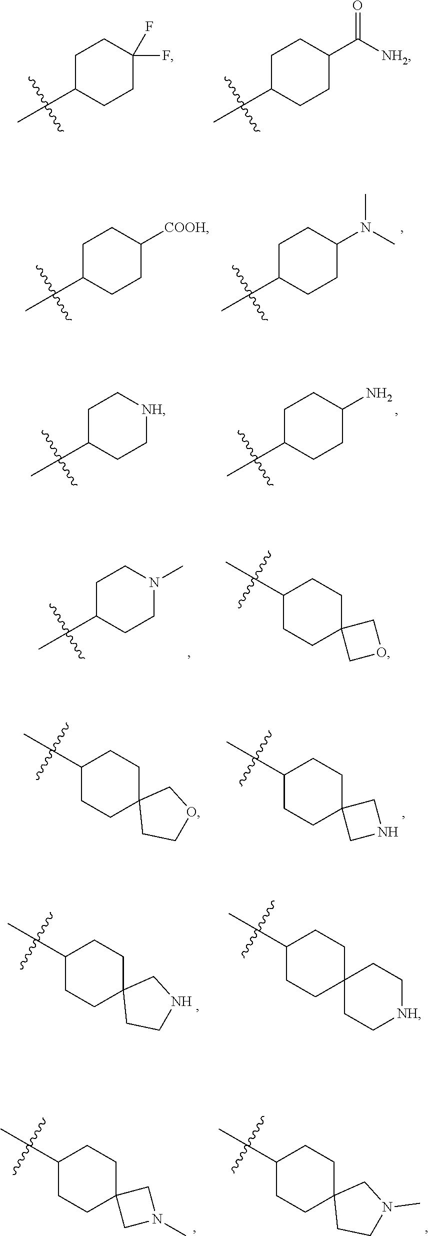

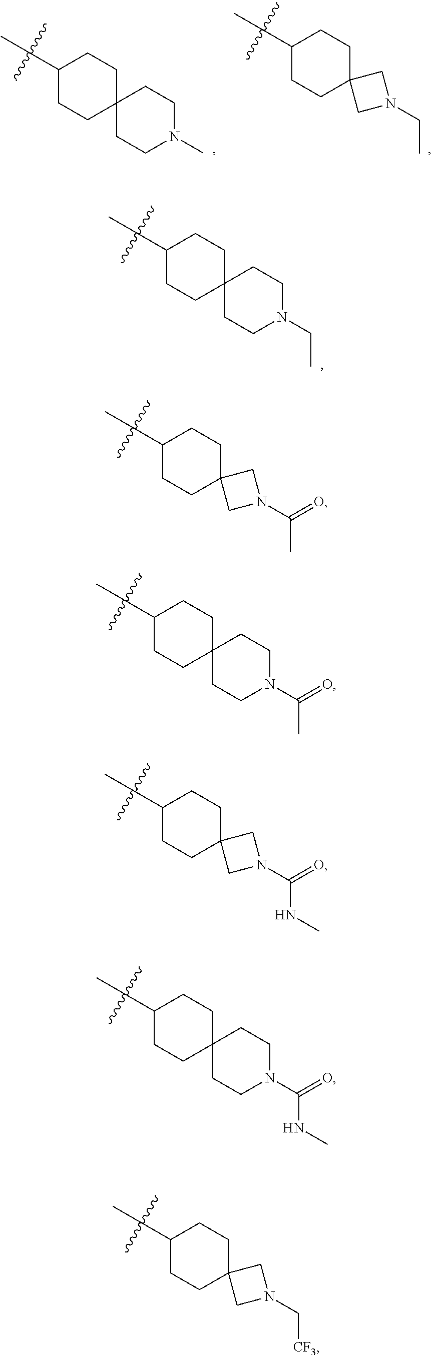

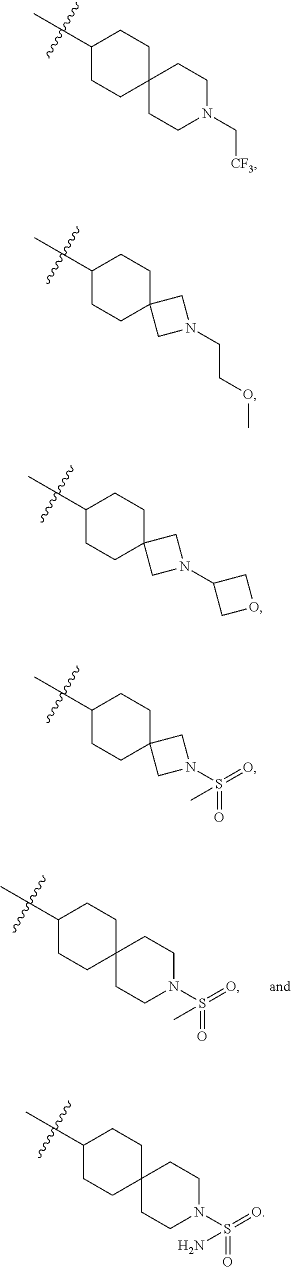 [SMILES: *C1CCC2(CC1)CCN(CC(F)(F)F)CC2 ; *C1CCC2(CC1)CN(CCOC)C2 ; *C1CCC2(CC1)CN(C1COC1)C2 ; *C1CCC2(CC1)CN(S(C)(=O)=O)C2 ; *C1CCC2(CC1)CCN(S(C)(=O)=O)CC2 ; *C1CCC2(CC1)CCN(S(N)(=O)=O)CC2]

66. The compound according to any one of claims 57 to 63, wherein moiety 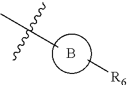
has the following structure: 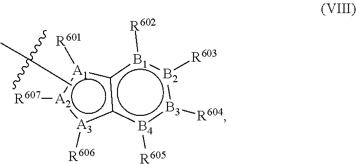
wherein A₁, A₂ and A₃ are independently selected from C, N, O and S; B₁, B₂ and B₃ are independently selected from C and N; B₄ is C, N or absent, and satisfies valence-bond saturation; R⁶⁰¹, R⁶⁰², R⁶⁰³, R⁶⁰⁴, R⁶⁰⁵, R⁶⁰⁶ and R⁶⁰⁷ may be appropriately absent; R⁶⁰¹, R⁶⁰², R⁶⁰³, R⁶⁰⁴, R⁶⁰⁵, R⁶⁰⁶ and R⁶⁰⁷ are independently selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl, wherein H at the carbon atoms may be substituted with one or more groups selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl;
preferably, moiety 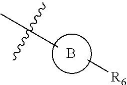
is selected from the following structure: 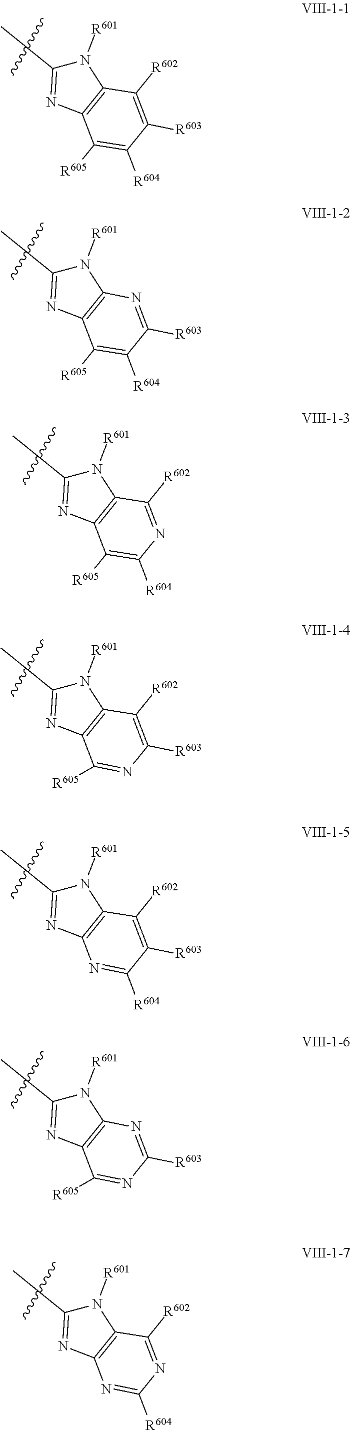 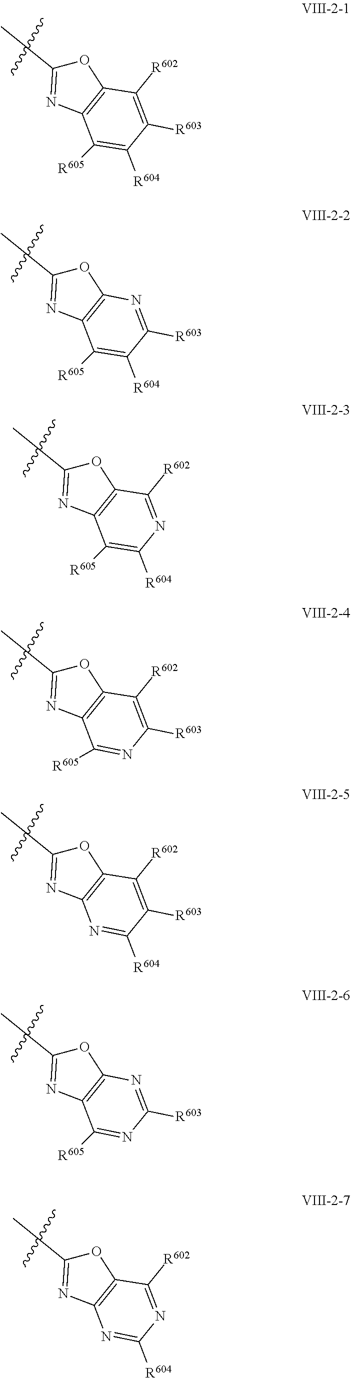 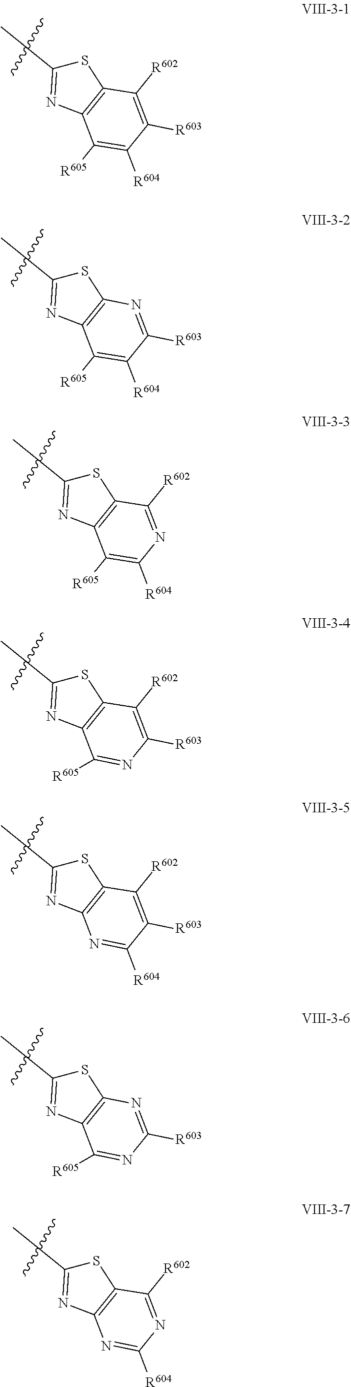 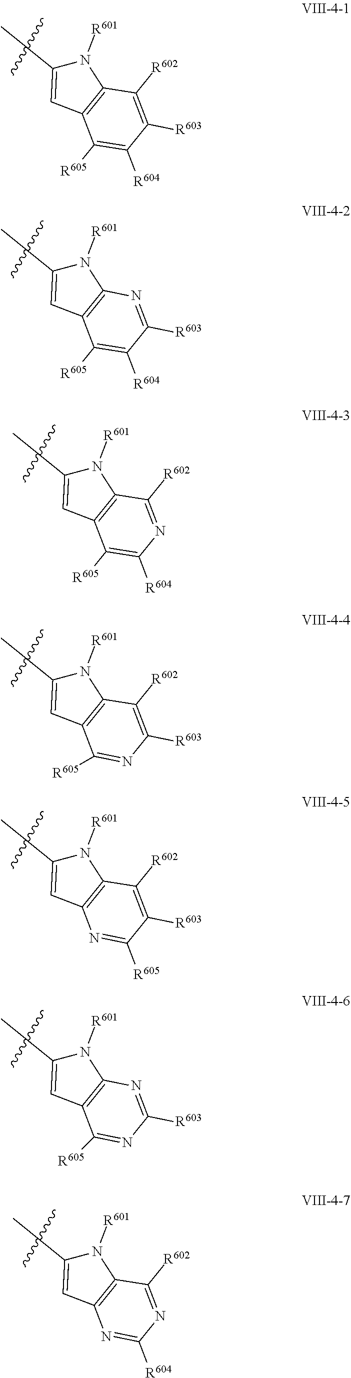 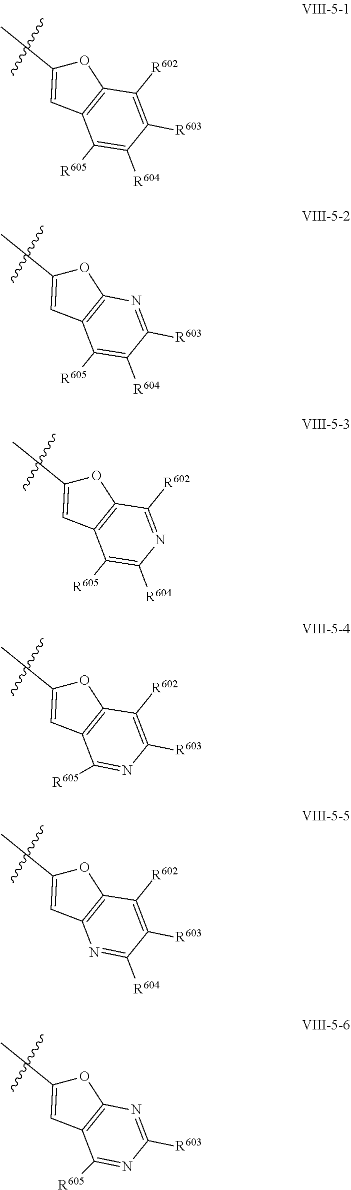 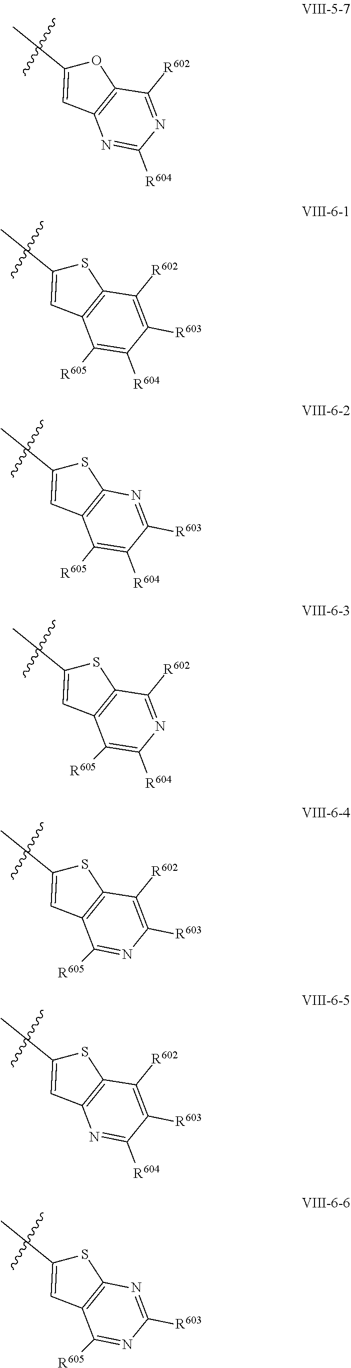 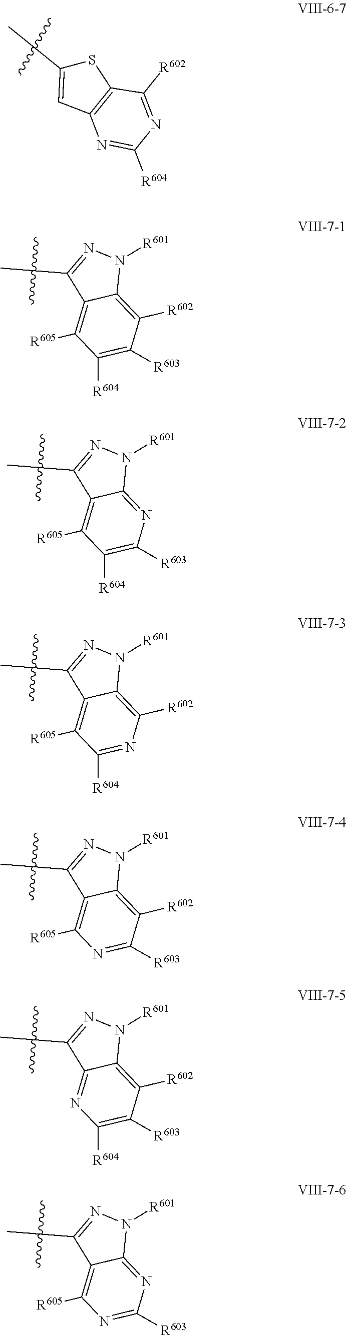 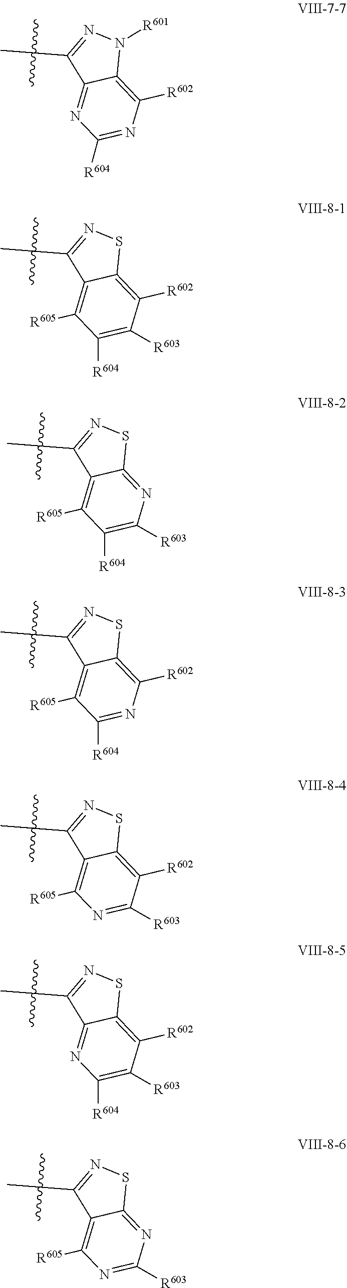 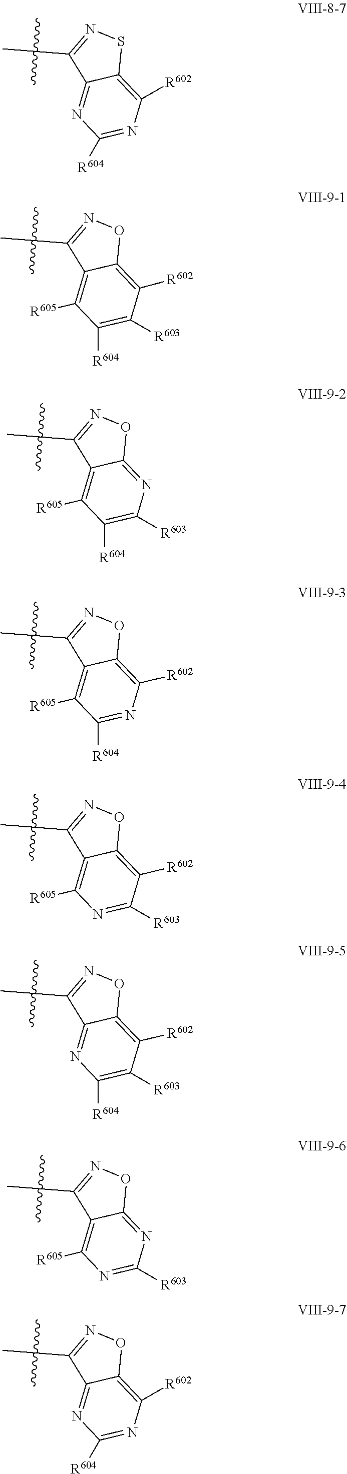 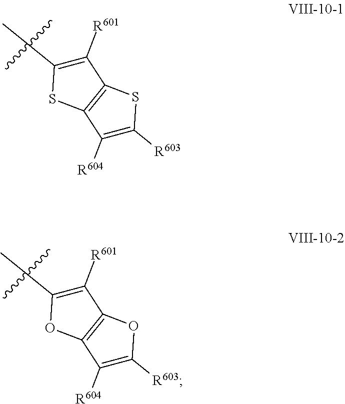
preferably, in formula VIII, R⁶⁰¹, R⁶⁰², R⁶⁰³, R⁶⁰⁴, R⁶⁰⁵, R⁶⁰⁶ and R⁶⁰⁷ are independently selected from: —H, -D, —F, —Cl, —Br, —I, —CF₃, —CHF₂, —CH₂F, —CH₂CF₃, —CN, —NO₂, —CH₃, 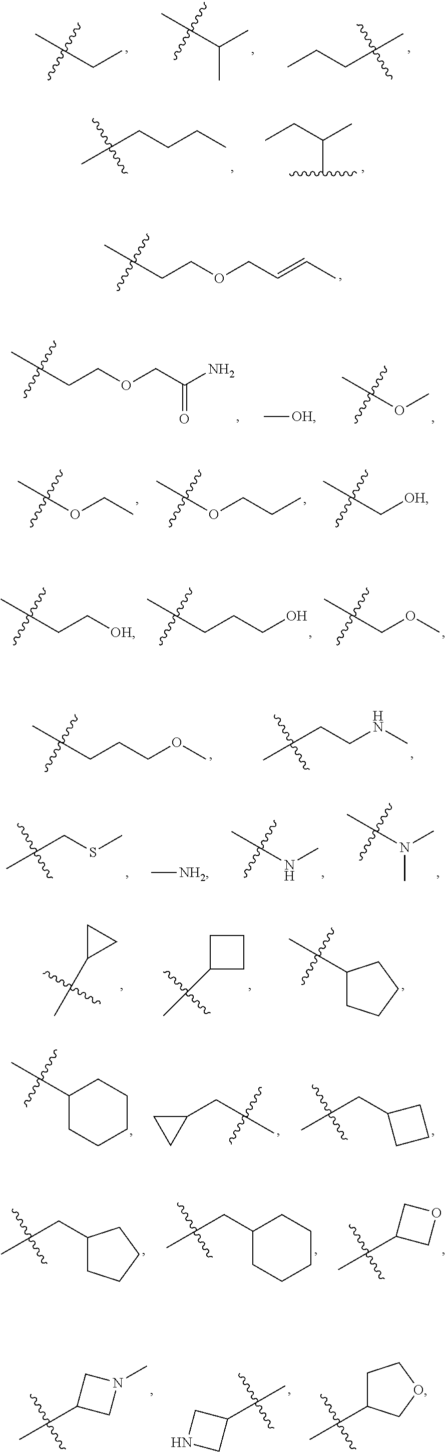 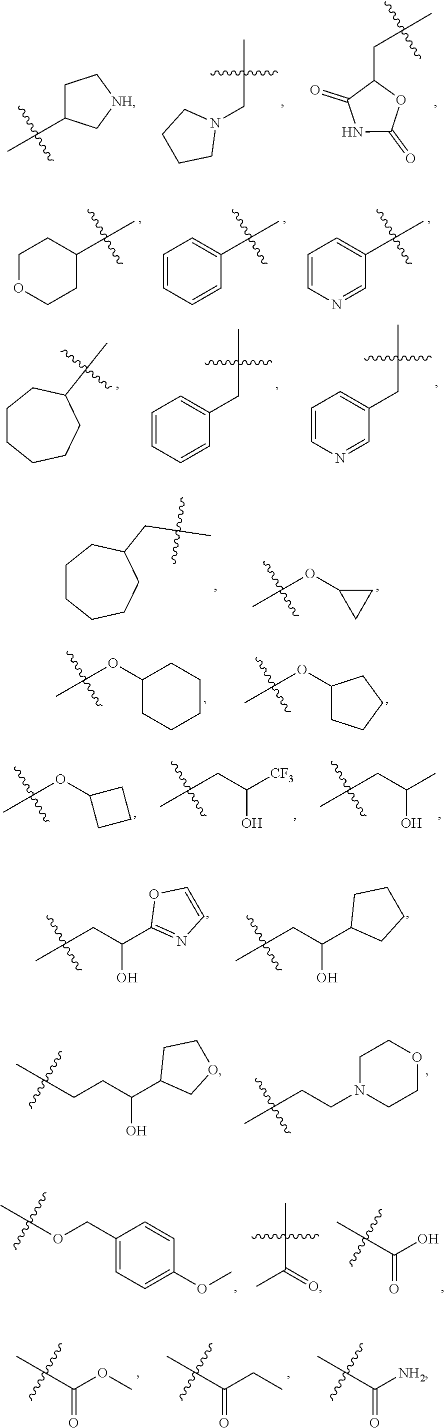 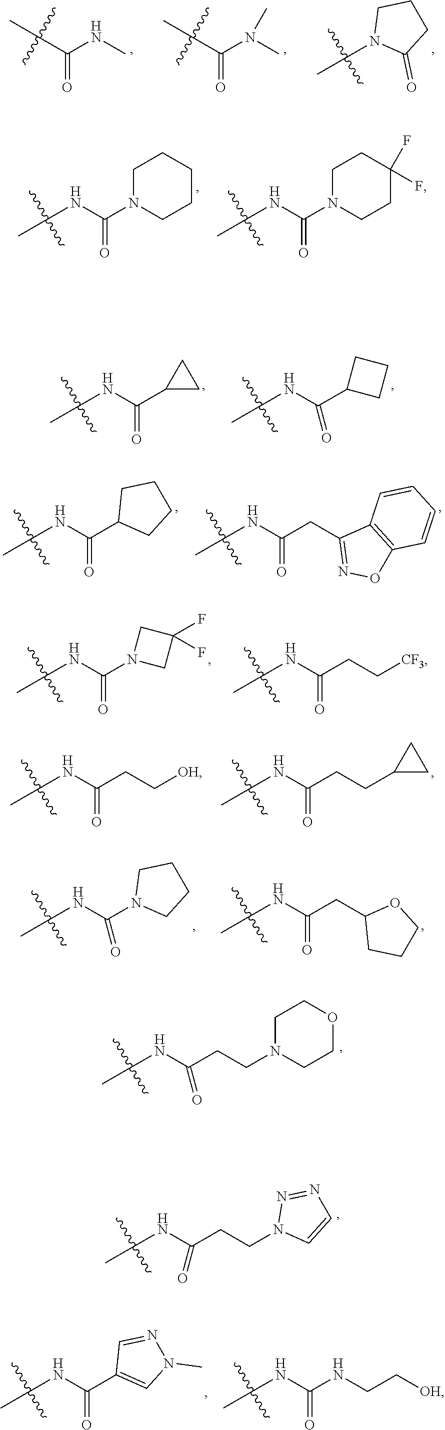 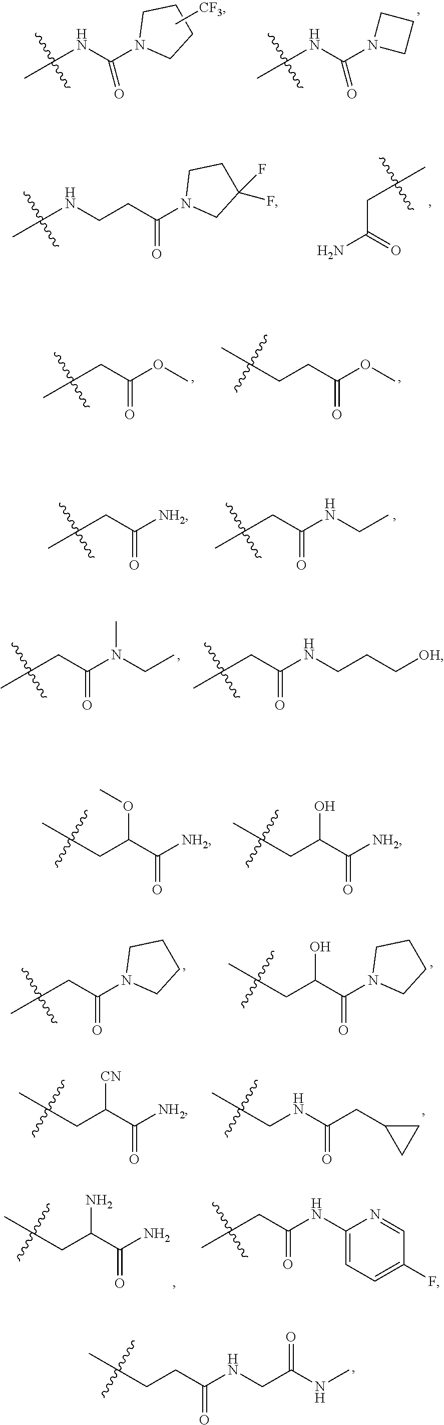 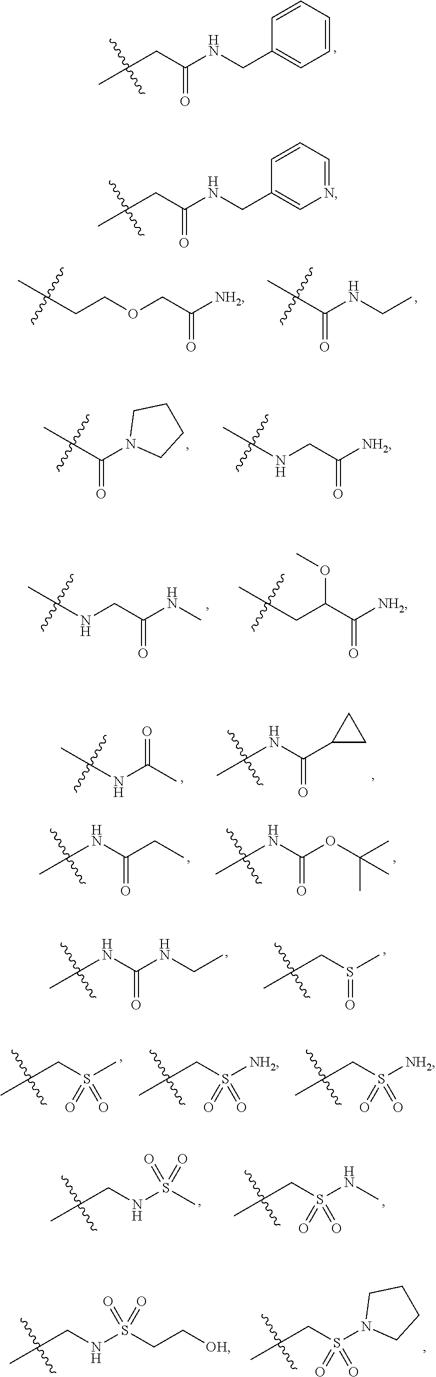 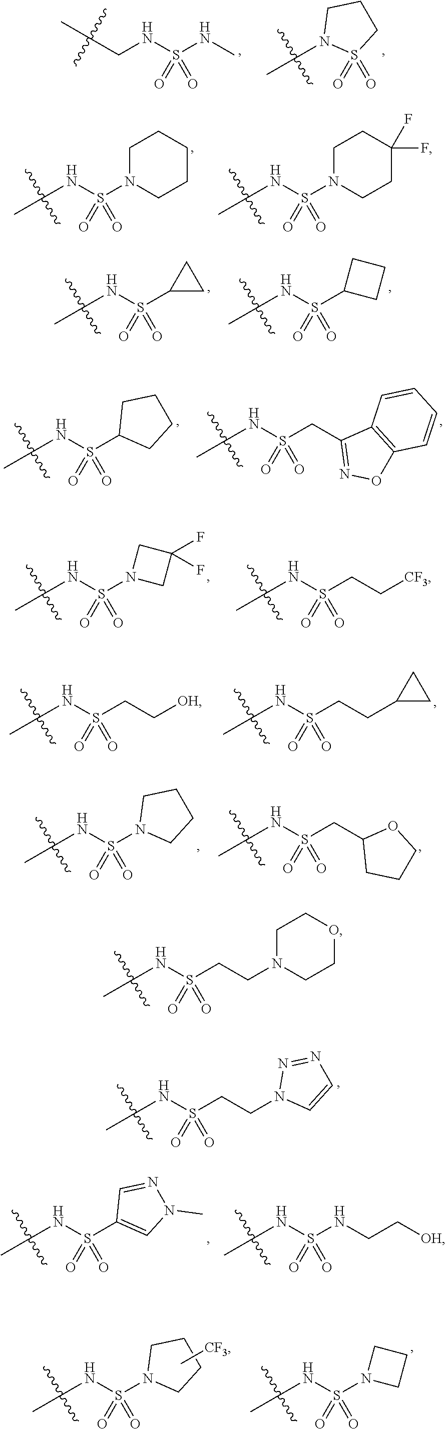 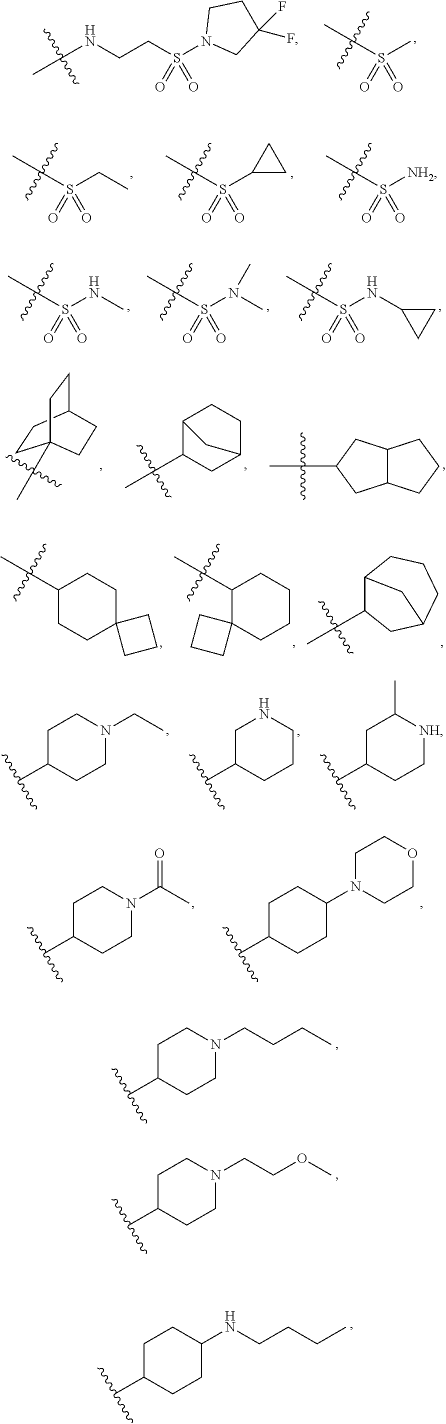 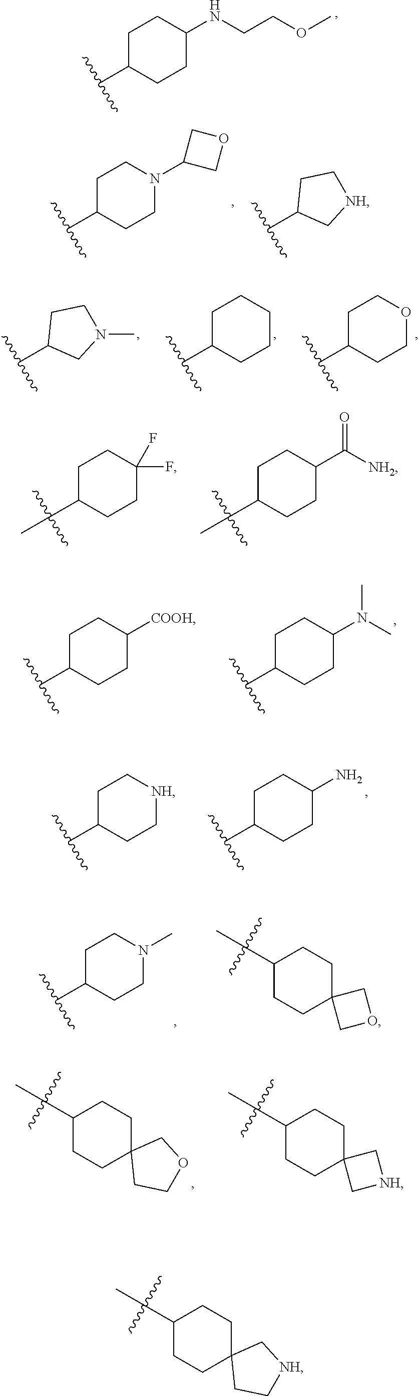 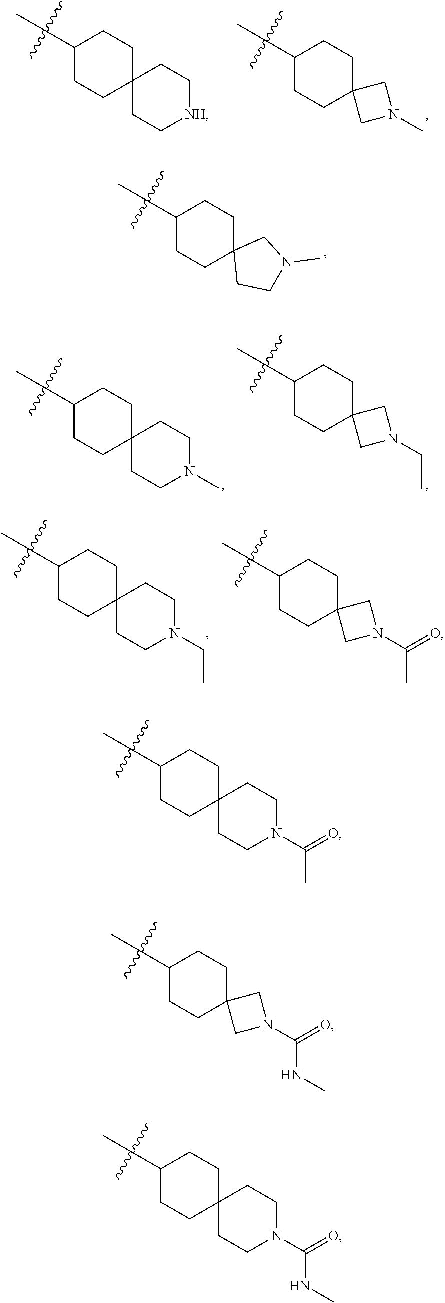 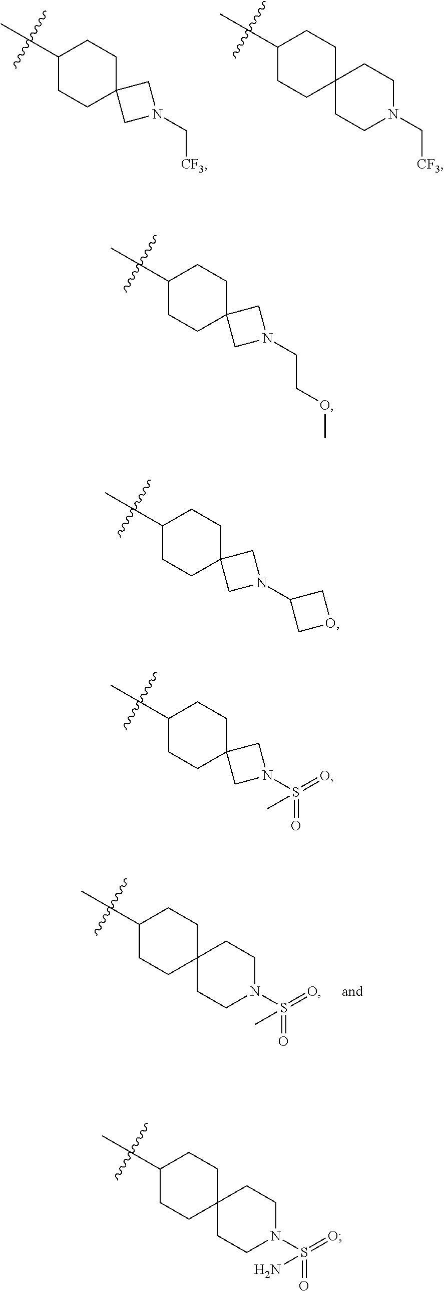
more preferably, R⁶⁰¹ is selected from: —H, C₁₋₆ alkyl (e.g., methyl, n-propyl or isopropyl), C₃₋₆ cycloalkyl (e.g., cyclopropyl, cyclobutyl, cyclopentyl, or cyclohexyl), and heterocycloalkyl 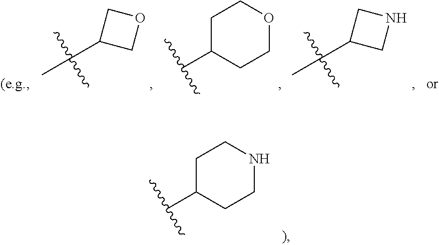 [SMILES: *C1COC1 ; *C1CCOCC1 ; *C1CNC1 ; *C1CCNCC1]
wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, and C₃₋₆ cycloalkyl; further preferably, R⁶⁰¹ is selected from: —H, C₁₋₃ alkyl, 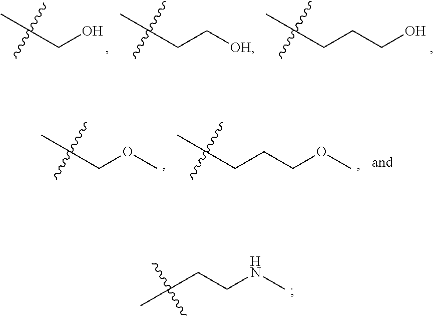
more preferably, R⁶⁰³ is selected from: —H, C₁₋₆ alkyl (e.g., methyl, ethyl, n-propyl, or isopropyl), —OH, C₁₋₆ alkoxy (e.g., methoxy, or ethoxy), —NH₂, C₁₋₆ alkylamino 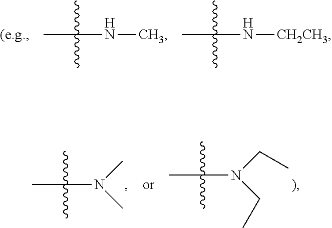
C₃₋₆ cycloalkyl (e.g. cyclopropyl, cyclobutyl, cyclopentyl, or cyclohexyl), and heterocycloalkyl 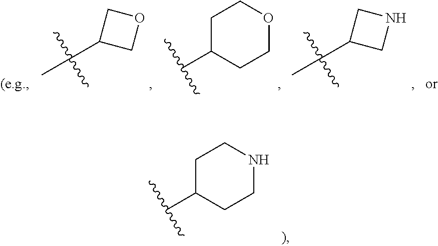 [SMILES: *C1COC1 ; *C1CCOCC1 ; *C1CNC1 ; *C1CCNCC1]
wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, and C₃₋₆ cycloalkyl; further preferably, R⁶⁰³ is selected from: —H, methoxy, and 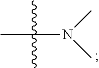 [SMILES: *N(C)C]
more preferably, R⁶⁰⁴ is selected from: —H, C₁₋₆ alkyl (e.g., methyl, ethyl, n-propyl, isopropyl), —OH, C₁₋₆ alkoxy (e.g., methoxy, or ethoxy), —NH₂, C₁₋₆ alkylamino 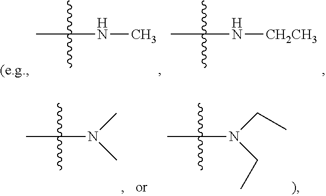
C₃₋₆ cycloalkyl (e.g., cyclopropyl, cyclobutyl, cyclopentyl, or cyclohexyl), heterocycloalkyl 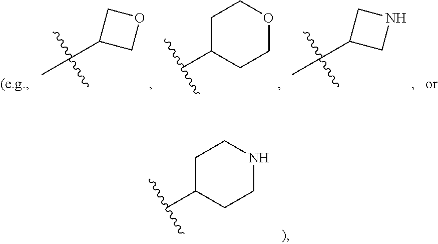 [SMILES: *C1COC1 ; *C1CCOCC1 ; *C1CNC1 ; *C1CCNCC1]
wherein one or more H in the C₁₋₆ alkyl, C₃₋₆ cycloalkyl or heterocycloalkyl may be substituted with a substituent selected from: halogen (particularly F), —OH, —NH₂, alkoxy, alkylamino, and C₃₋₆ cycloalkyl; further preferably, R⁶⁰⁴ is selected from: —H, methoxy, and

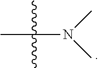 [SMILES: *N(C)C]

67. The compound according to any one of claims 57 to 63, wherein moiety 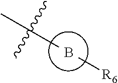
has the following structure: 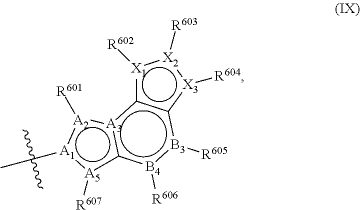
wherein A₁, A₂ and A₃ are independently selected from C, N, O and S; B₁, B₂ and B₃ are independently selected from C and N; B₄ is C, N or absent; X₁, X₂ and X₃ are independently selected from C, N, O and S, and satisfy valence-bond saturation; R⁶⁰¹, R⁶⁰², R⁶⁰³, R⁶⁰⁴, R⁶⁰⁵, R⁶⁰⁶ and R⁶⁰⁷ may be appropriately absent;
R⁶⁰¹, R⁶⁰², R⁶⁰³, R⁶⁰⁴, R⁶⁰⁵, R⁶⁰⁶ and R⁶⁰⁷ are independently selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl, wherein H at the carbon atoms may be substituted with one or more groups selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl;
preferably, moiety 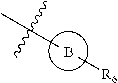
is selected from the following structures: 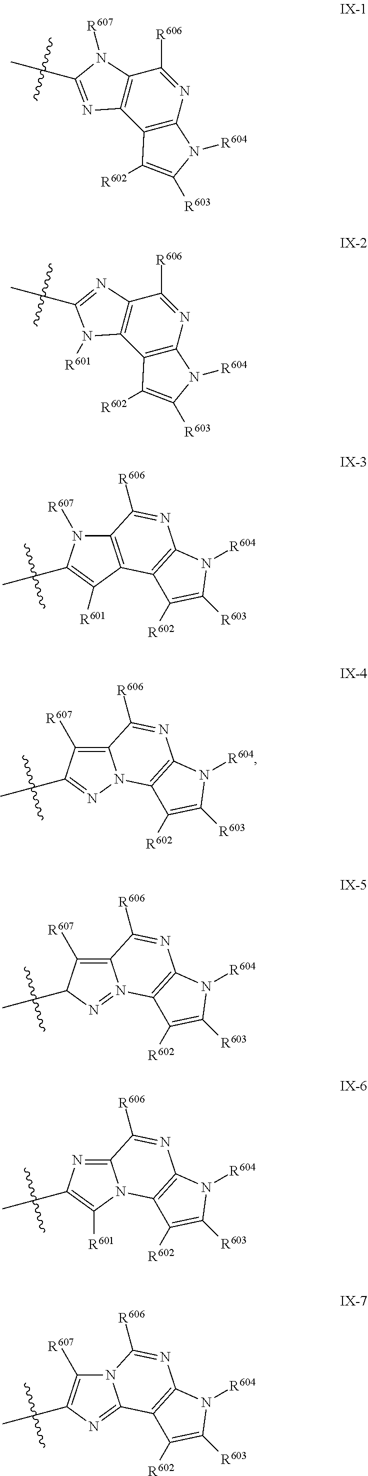 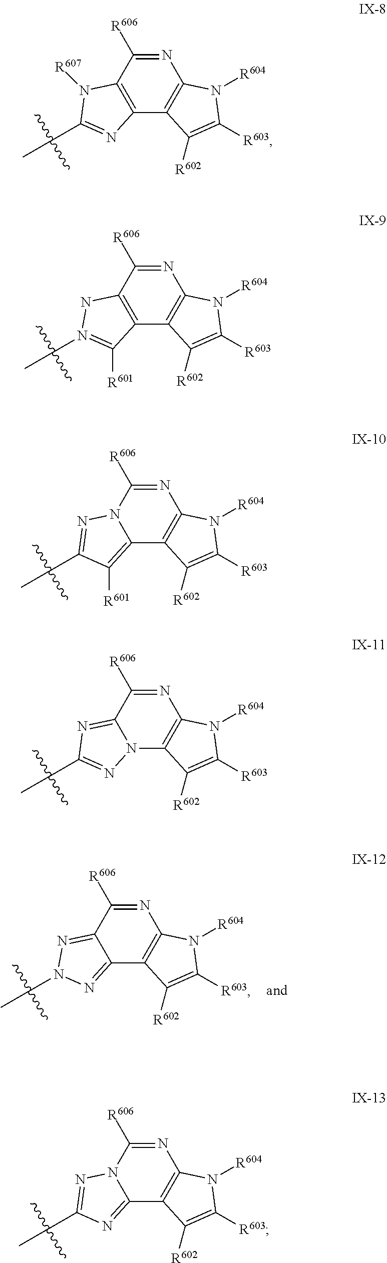
preferably, R⁶⁰¹, R⁶⁰², R⁶⁰³, R⁶⁰⁴, R⁶⁰⁵, R⁶⁰⁶ and R⁶⁰⁷ are independently selected from: —H, -D, —F, —Cl, —Br, —I, —CF₃, —CHF₂, —CH₂F, —CH₂CF₃, —CN, —NO₂, —CH₃, 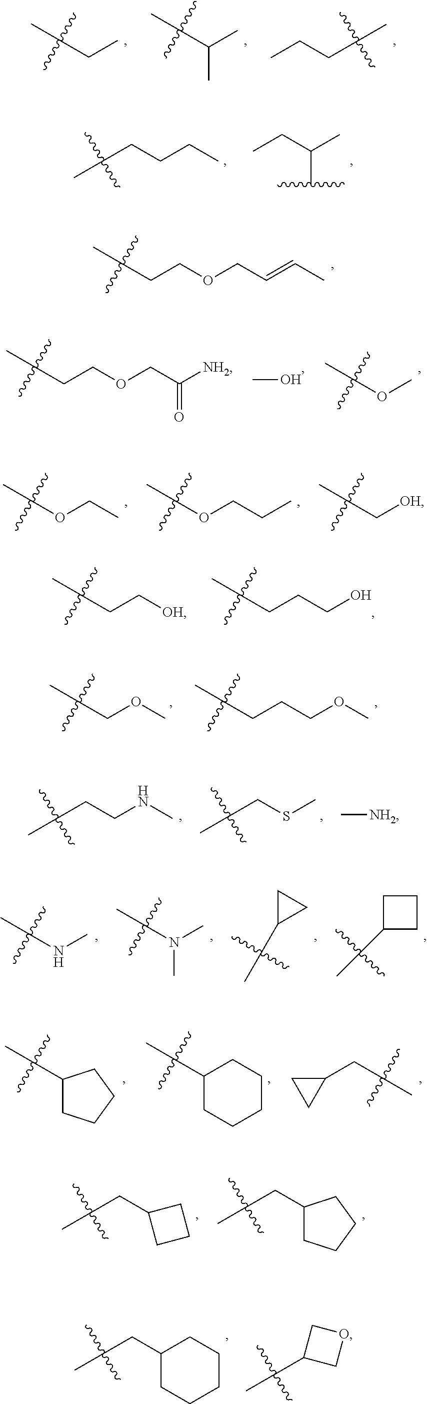 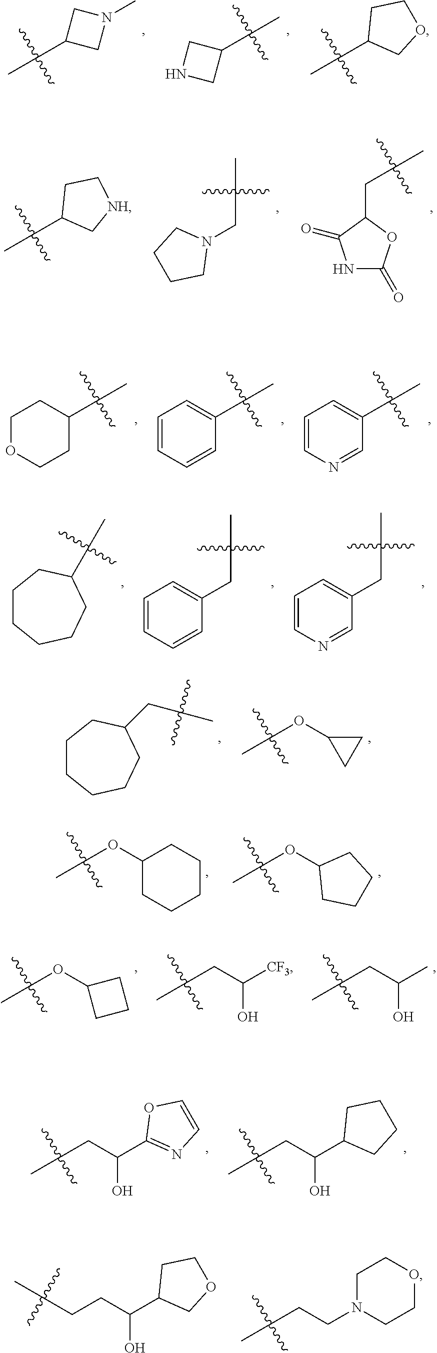 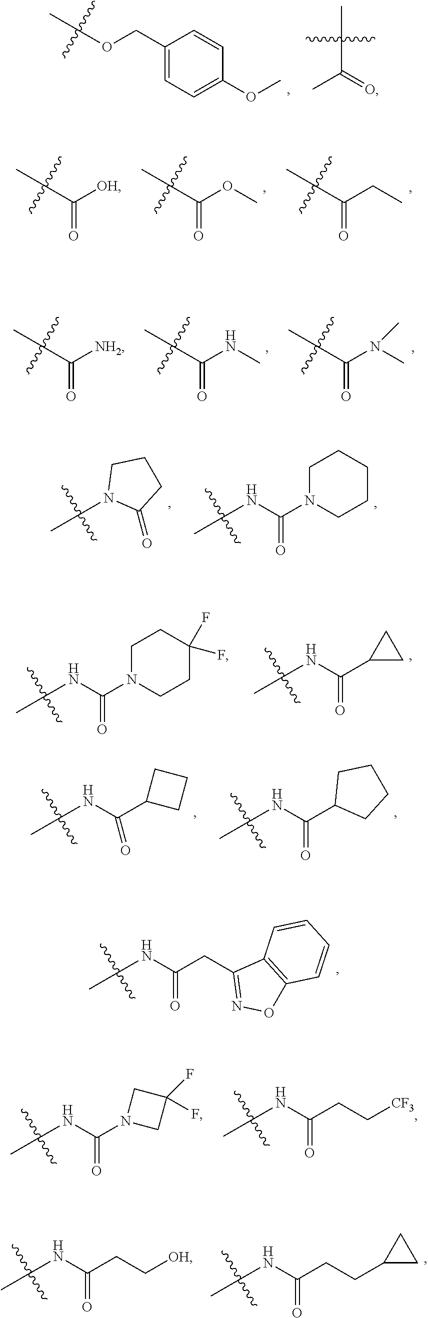 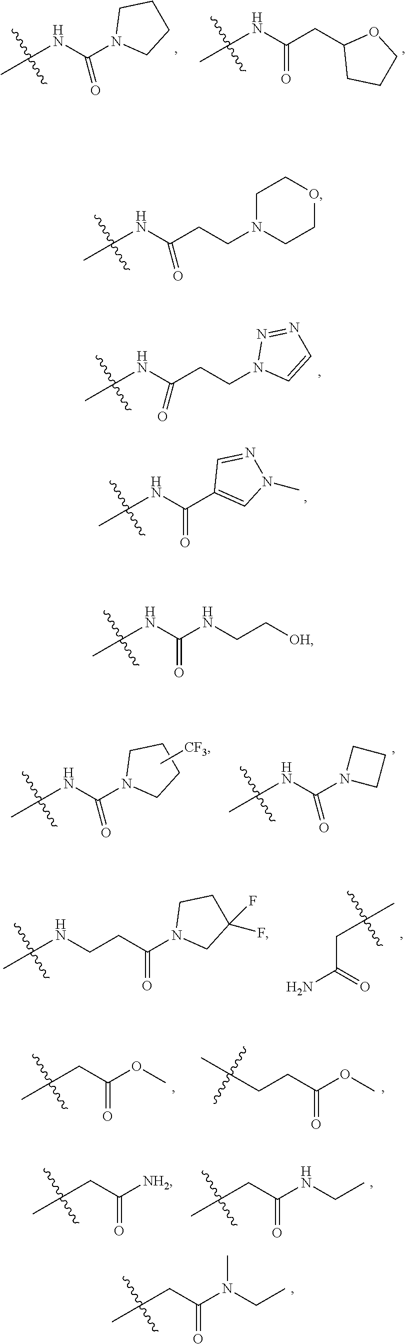 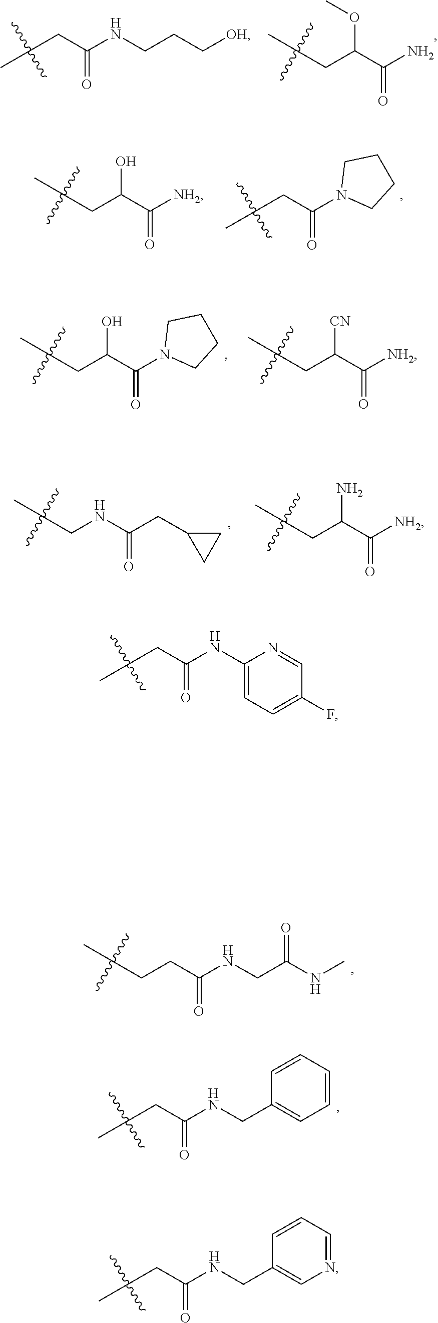 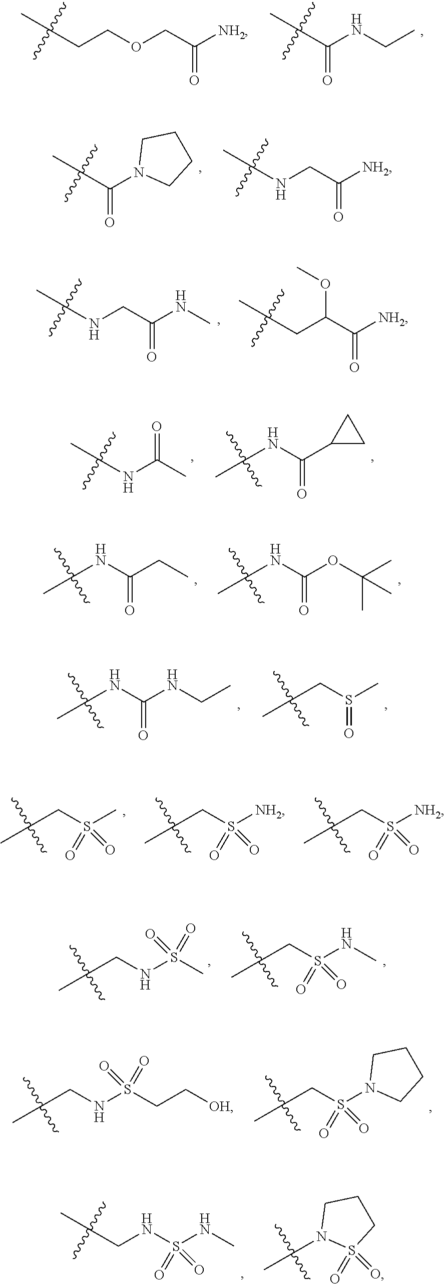 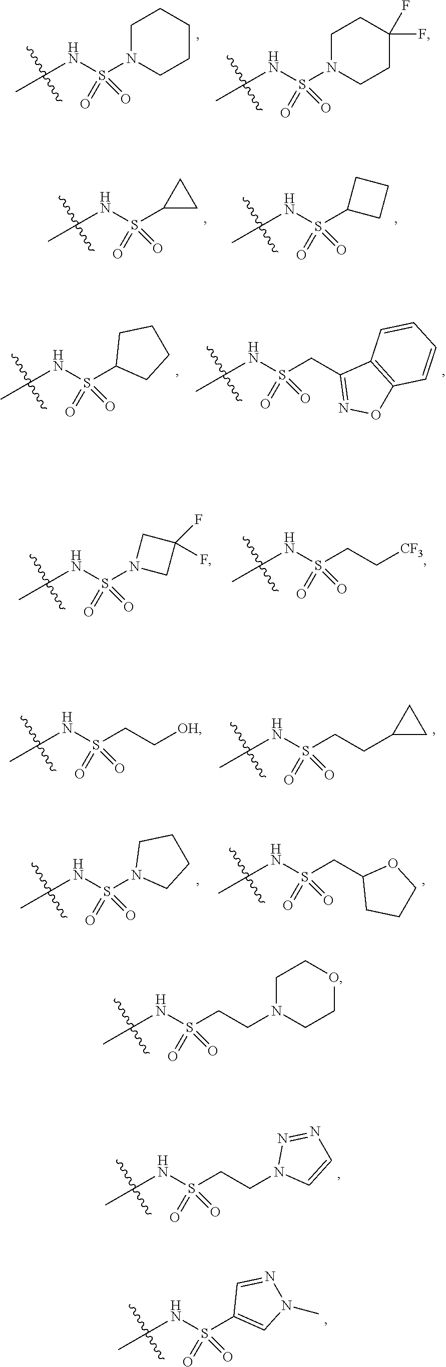 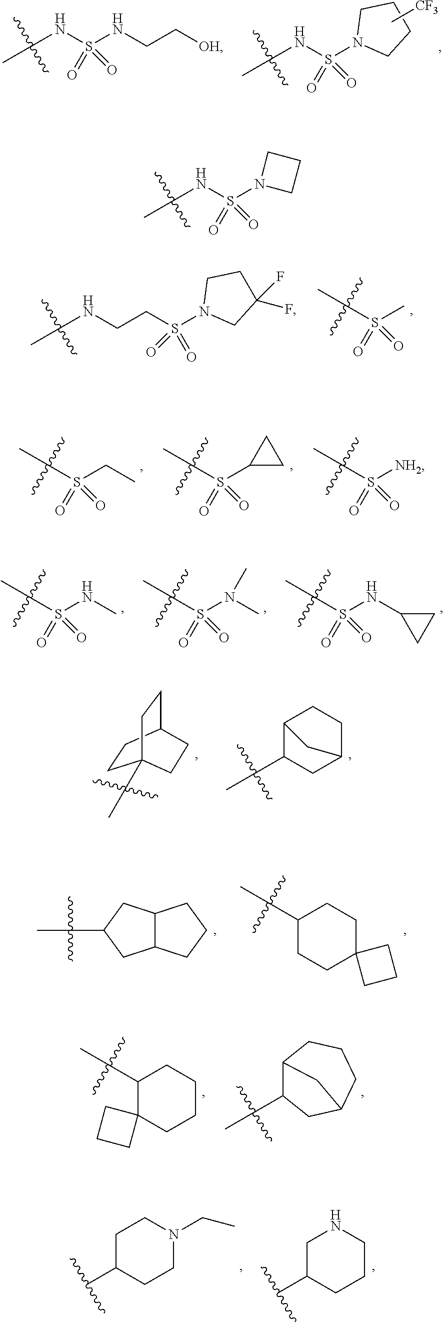 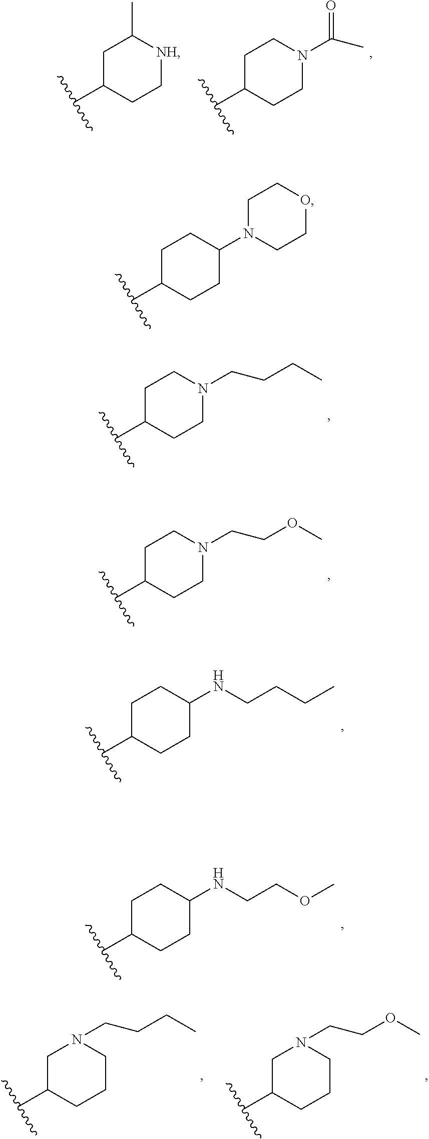 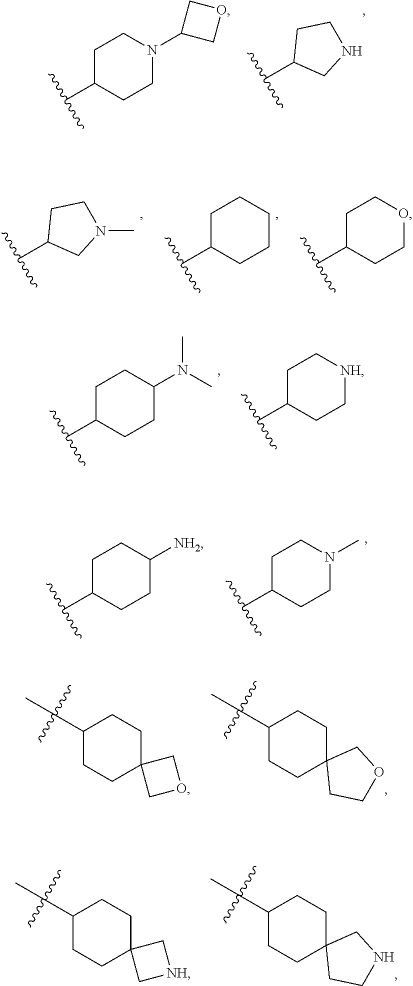 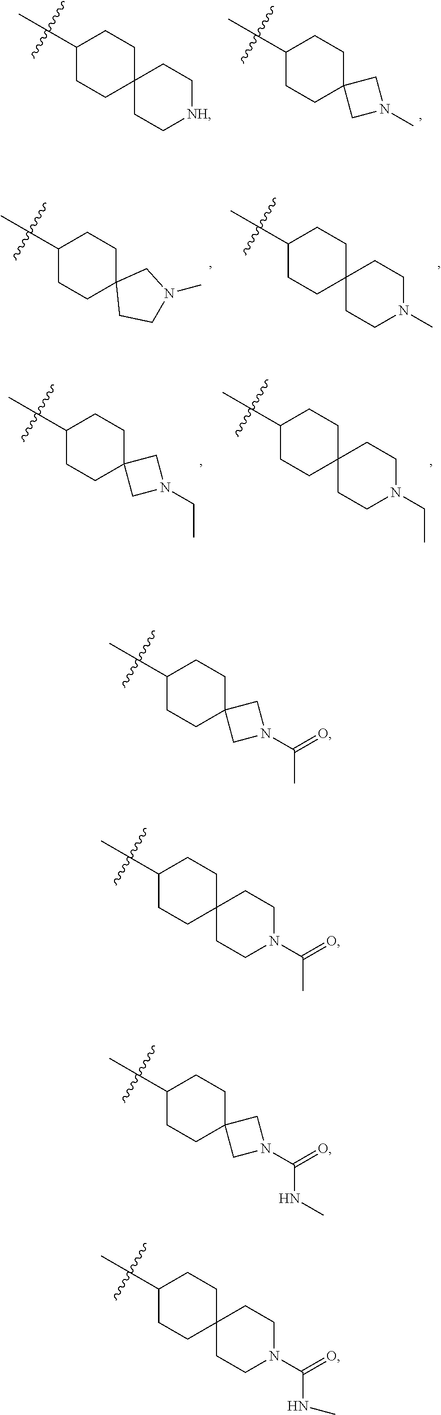 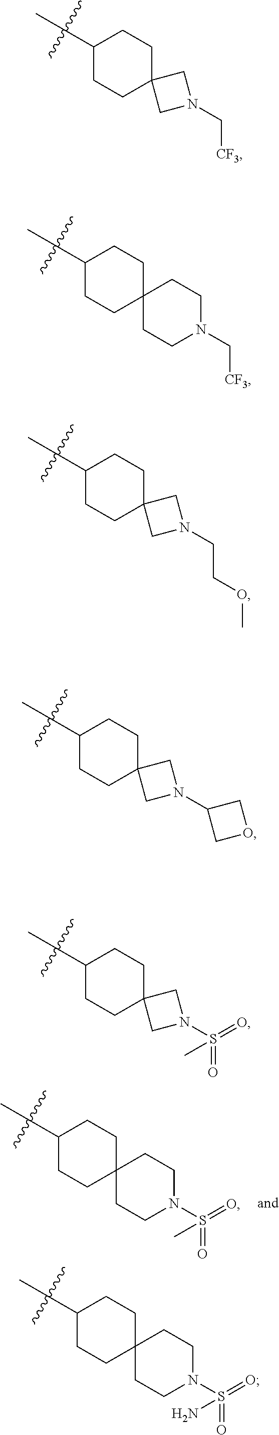
more preferably, R⁶⁰¹ is selected from: —H, C₁₋₆ alkyl (e.g., methyl, ethyl, n-propyl, or isopropyl), and C₃₋₆ cycloalkyl (e.g., cyclopropyl, cyclobutyl, cyclopentyl, or cyclohexyl);
more preferably, R⁶⁰⁴ is selected from: —H, C₁₋₆ alkyl (e.g., methyl, ethyl, n-propyl, or isopropyl), and C₃₋₆ cycloalkyl (e.g., cyclopropyl, cyclobutyl, cyclopentyl, or cyclohexyl);
more preferably, R⁶⁰⁷ is selected from: —H, C₁₋₆ alkyl (e.g., methyl, ethyl, n-propyl, or isopropyl), and C₃₋₆ cycloalkyl (e.g., cyclopropyl, cyclobutyl, cyclopentyl, or cyclohexyl);
more preferably, R² is selected from:

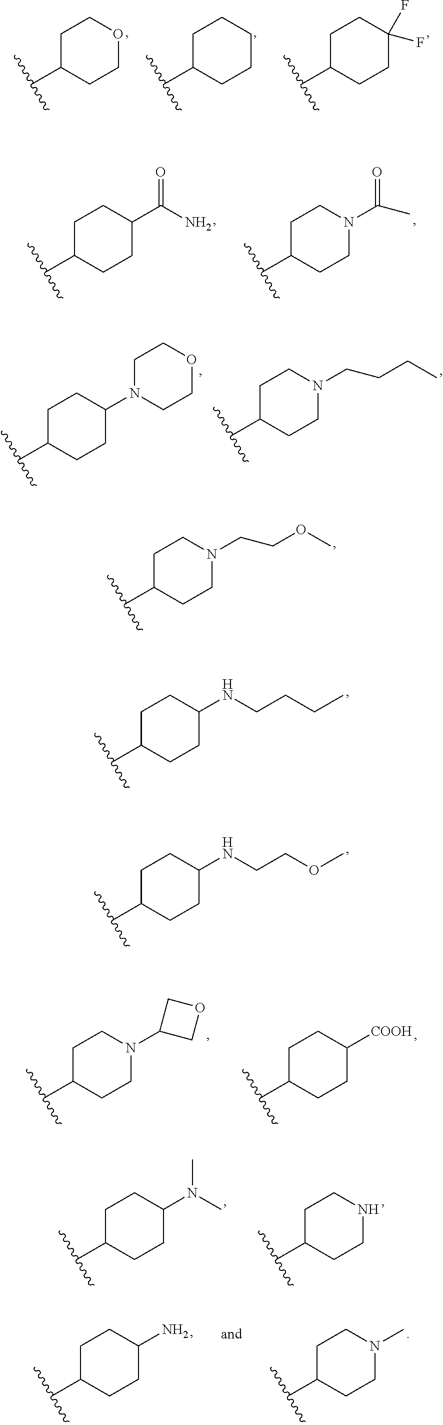

68. The compound according to any one of claims 57 to 63, wherein 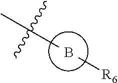
is selected from: 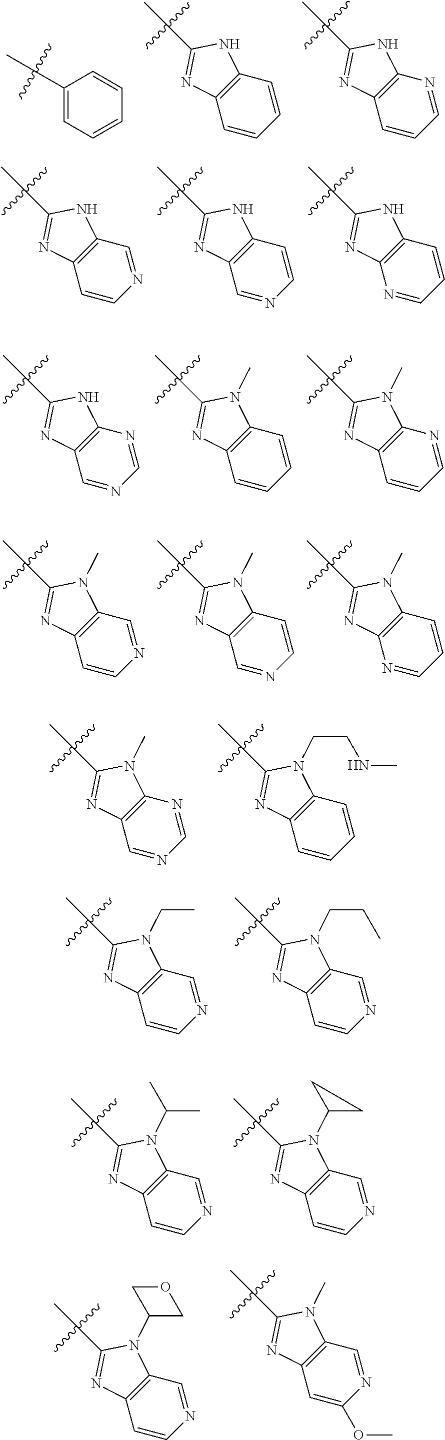 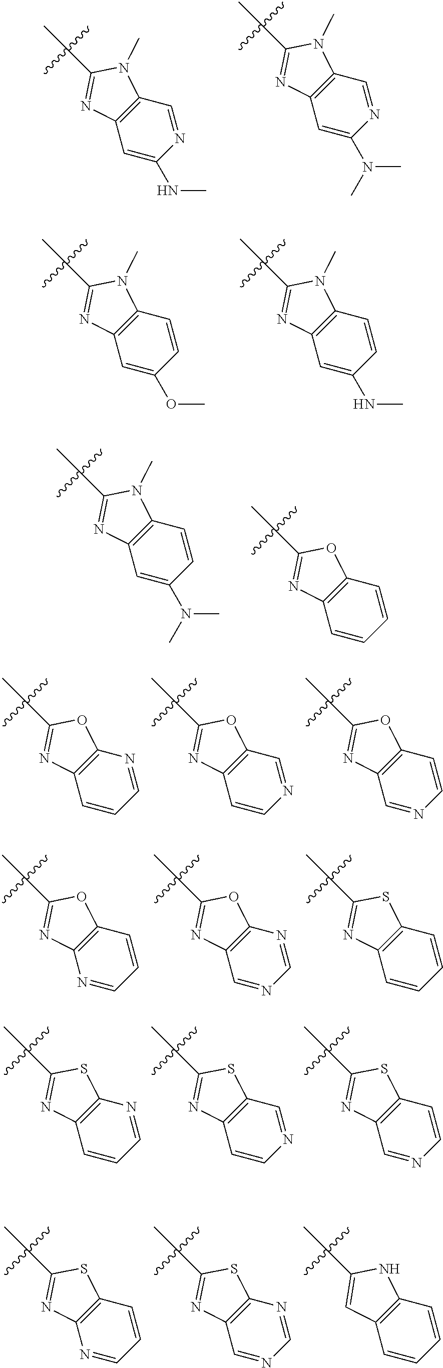 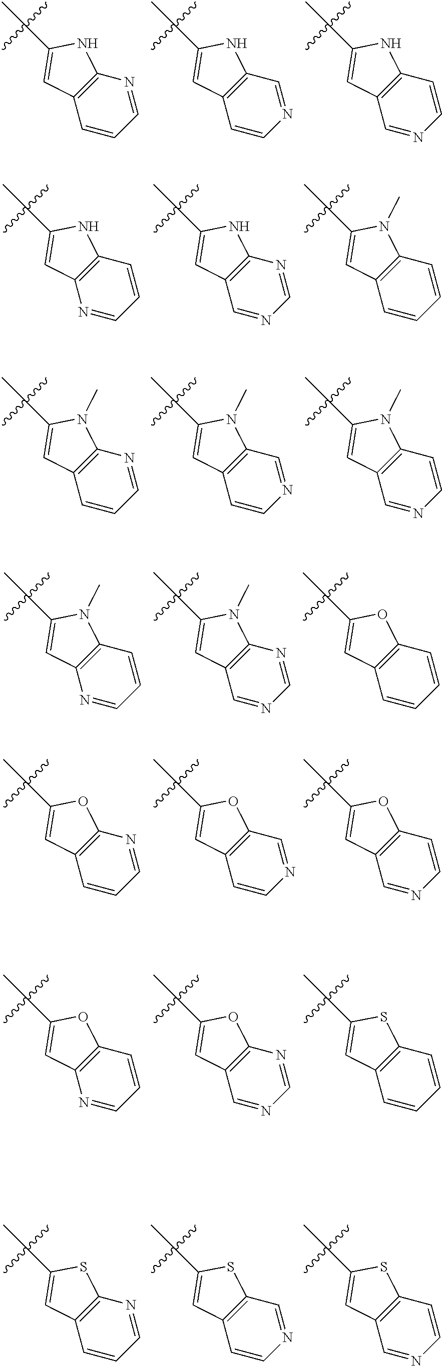 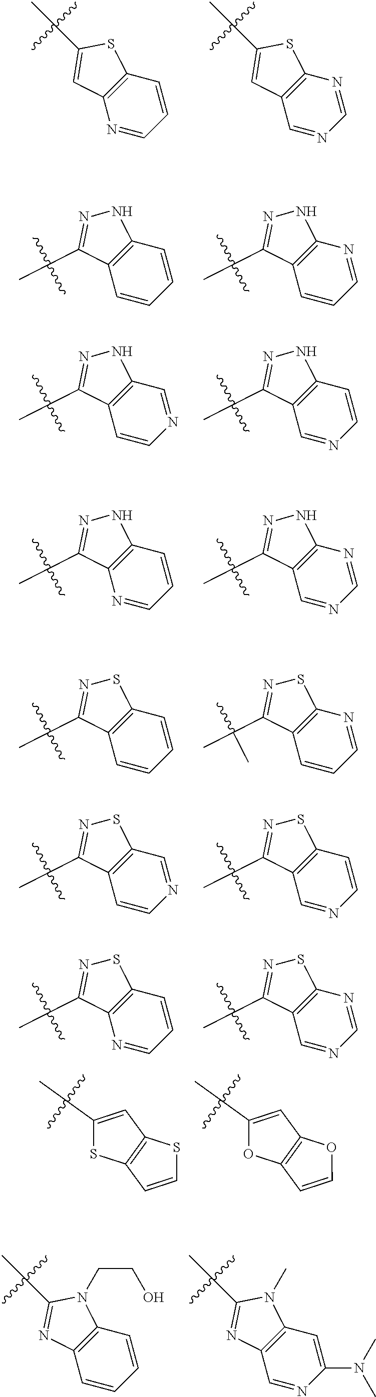 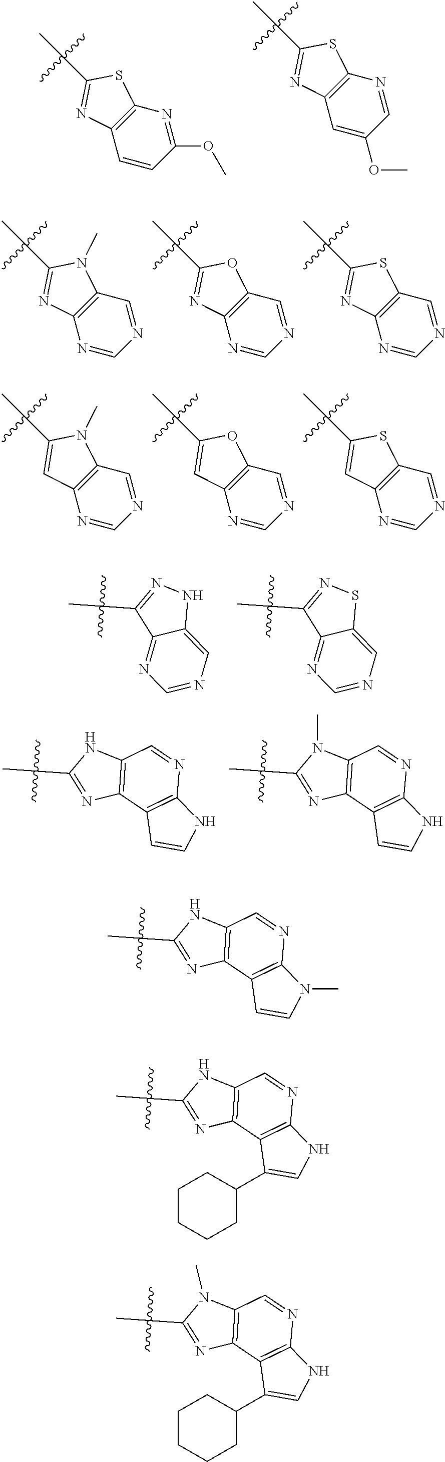 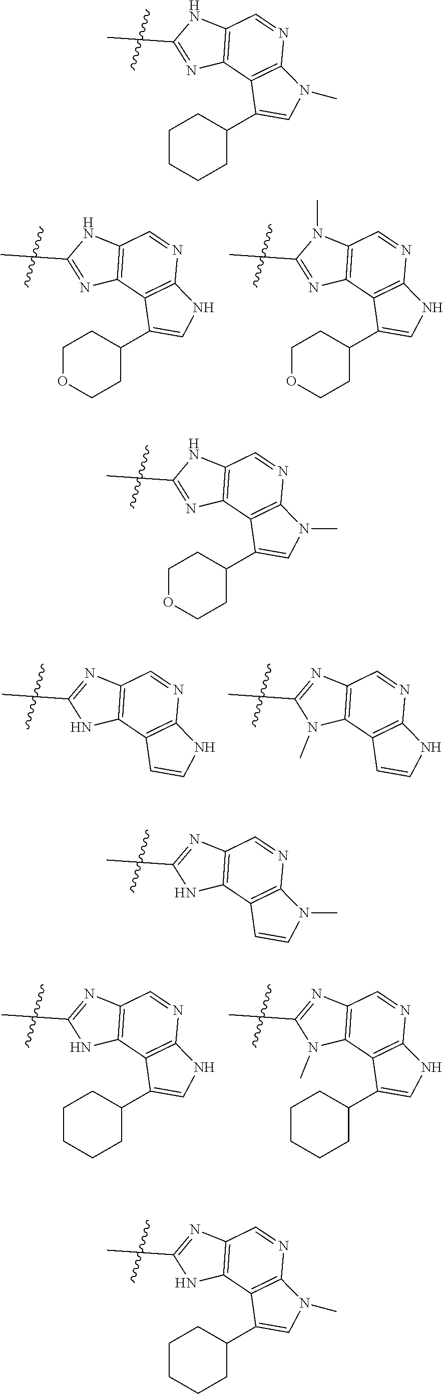 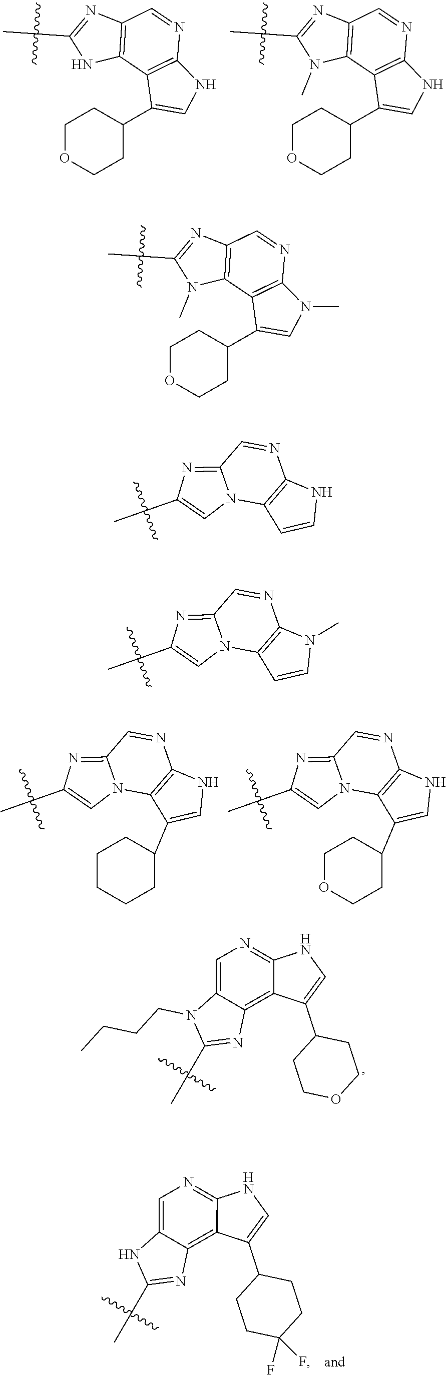 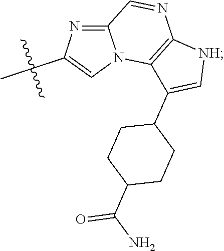 [SMILES: *c1cn2c(cnc3[nH]cc(C4CCC(C(N)=O)CC4)c32)n1]
wherein H linked to carbon atoms may be optionally substituted with one or more groups selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, heterocycloalkyl, and heteroaryl.

69. The compound according to claim 1, wherein R₁ is selected from: H, halogen, —CN, —NO₂, —CF₃, —OCF₃, C₁₋₆ alkyl, —O(C₁₋₆ alkyl), —N(C₁₋₆ alkyl)(C₁₋₆ alkyl), —COOH, —COO(C₁₋₆ alkyl), —OCOH, —OCO(C₁₋₆ alkyl), —CONH(C₁₋₆ alkyl), —CON(C₁₋₆ alkyl)(C₁₋₆ alkyl),

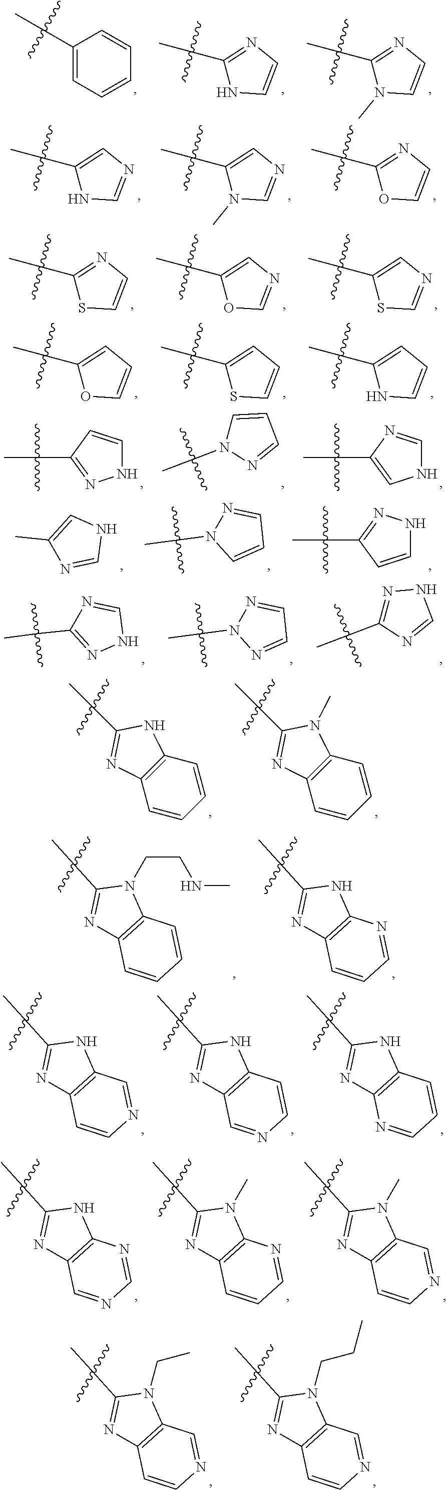

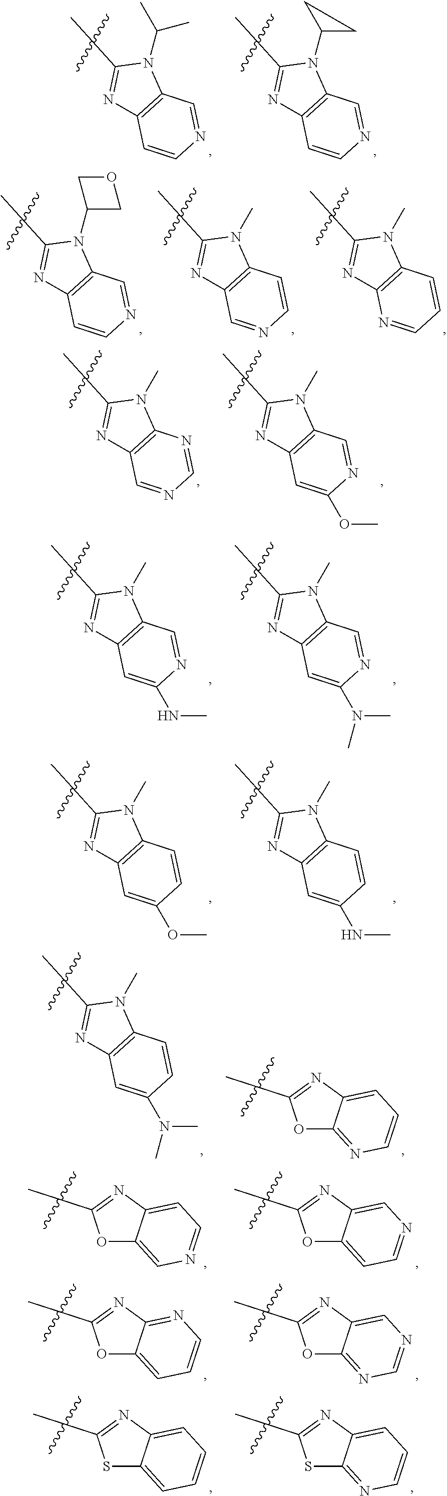

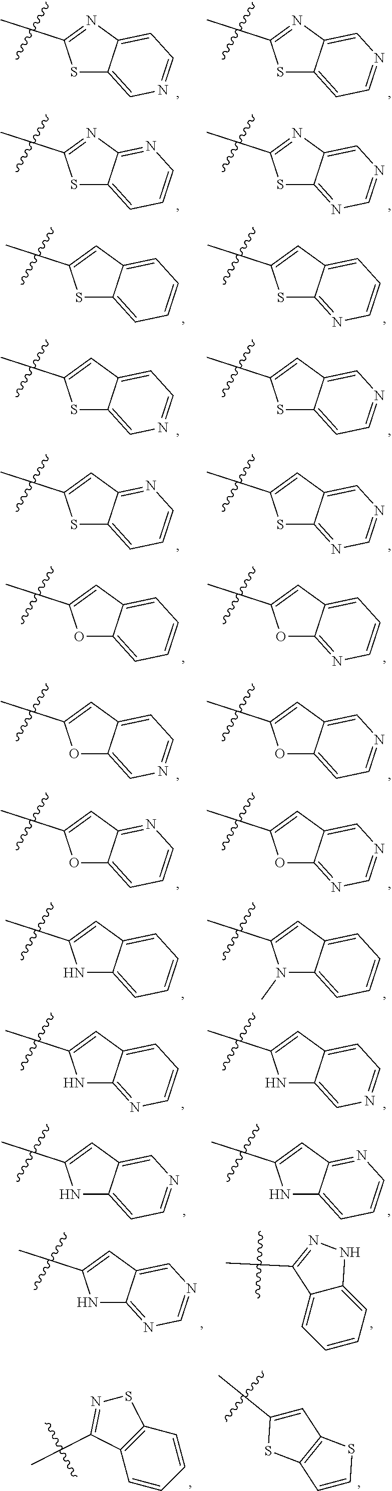

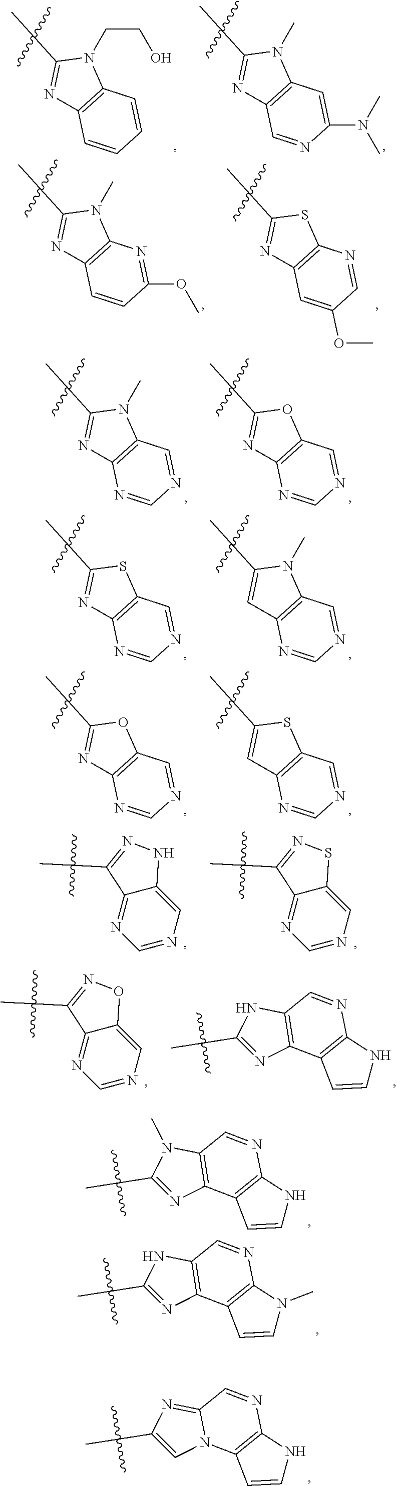

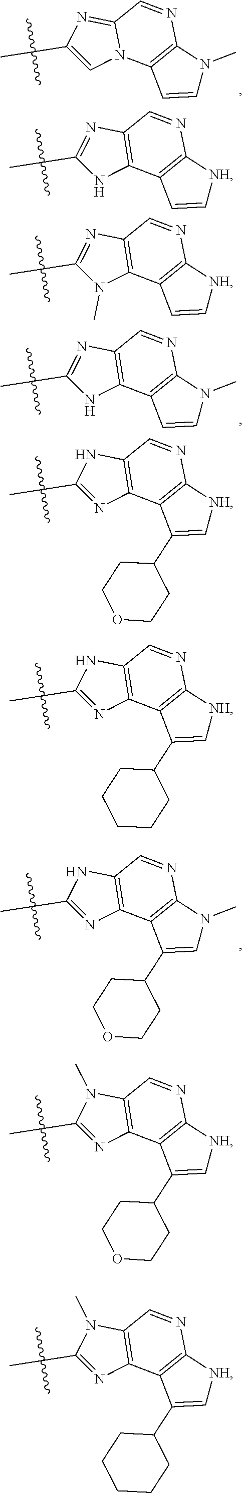

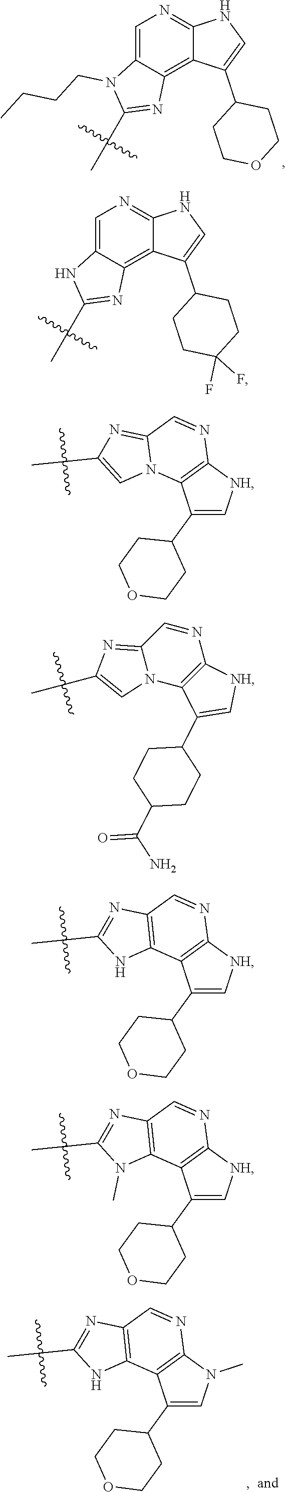

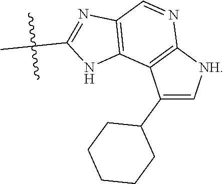 [SMILES: *c1nc2cnc3[nH]cc(C4CCCCC4)c3c2[nH]1]

70. The compound according to any one of claims 57 to 69, wherein each -Cy- is independently selected from: 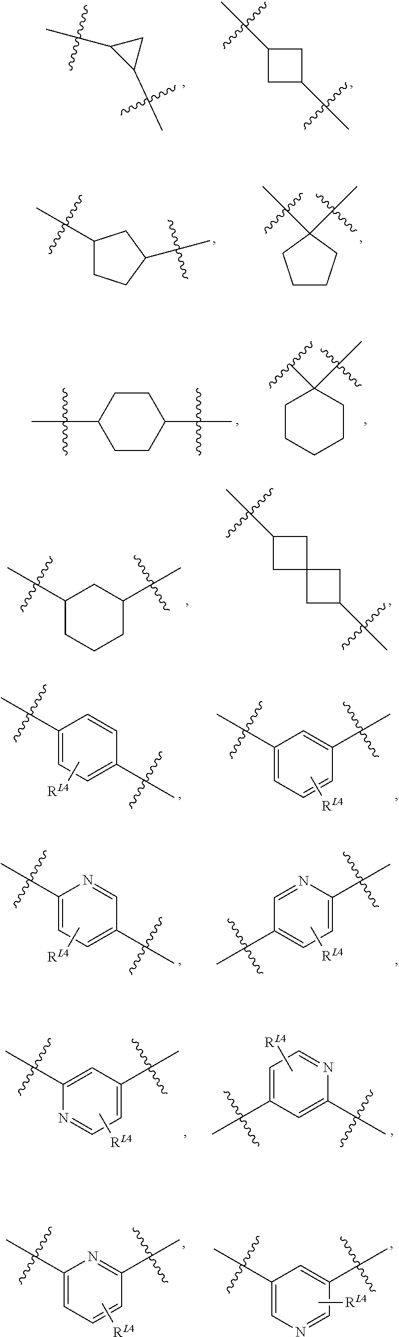 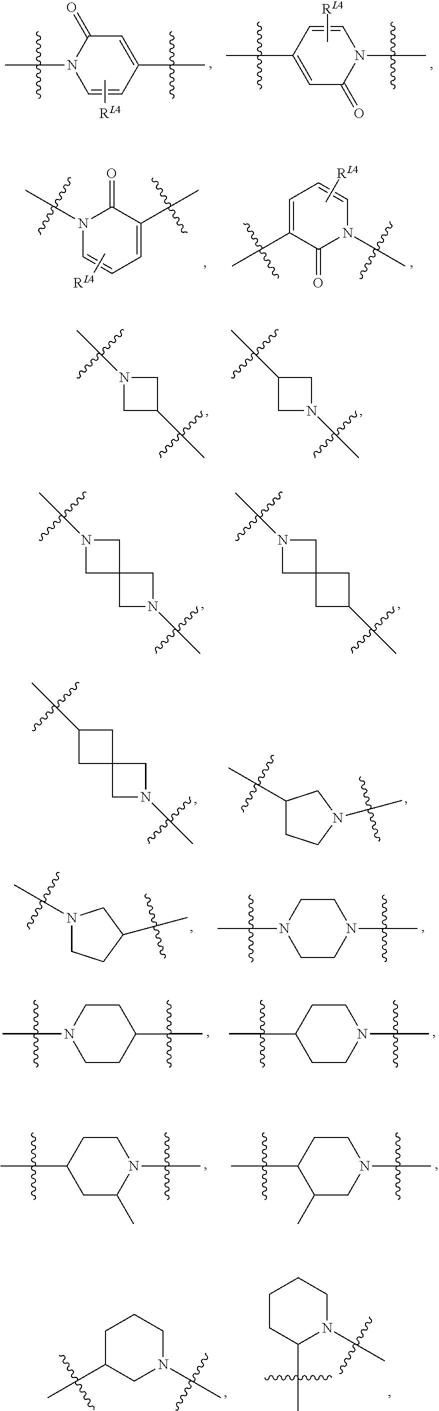 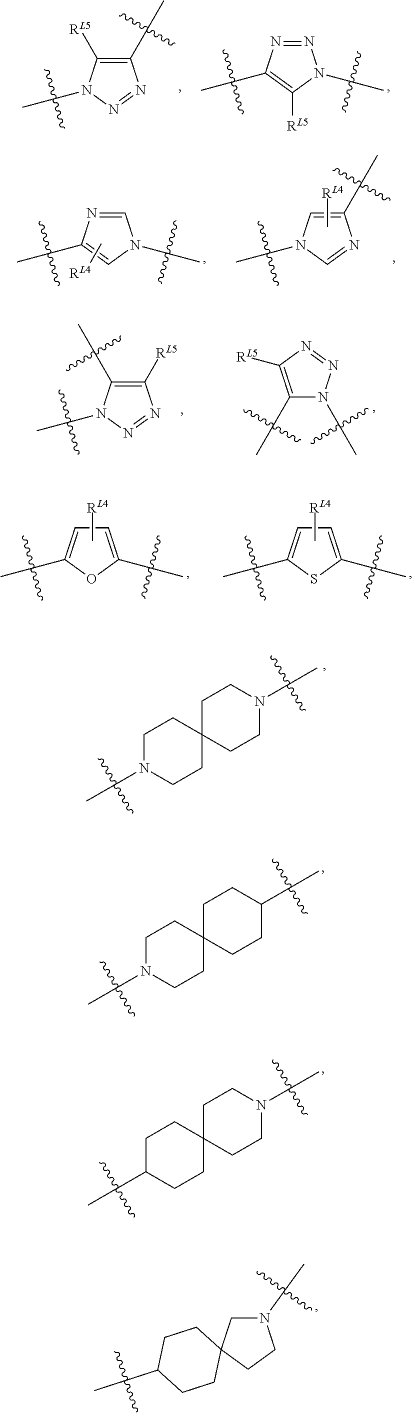 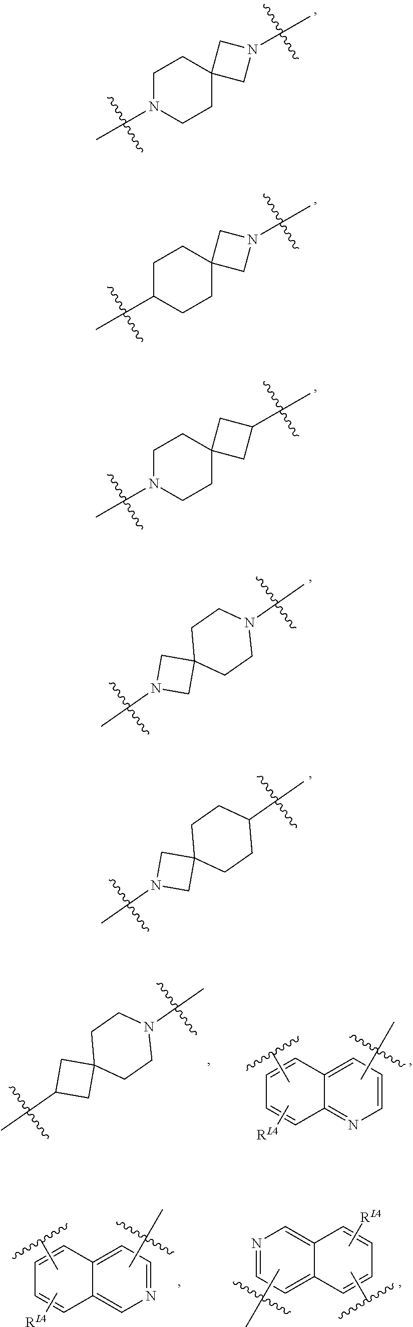 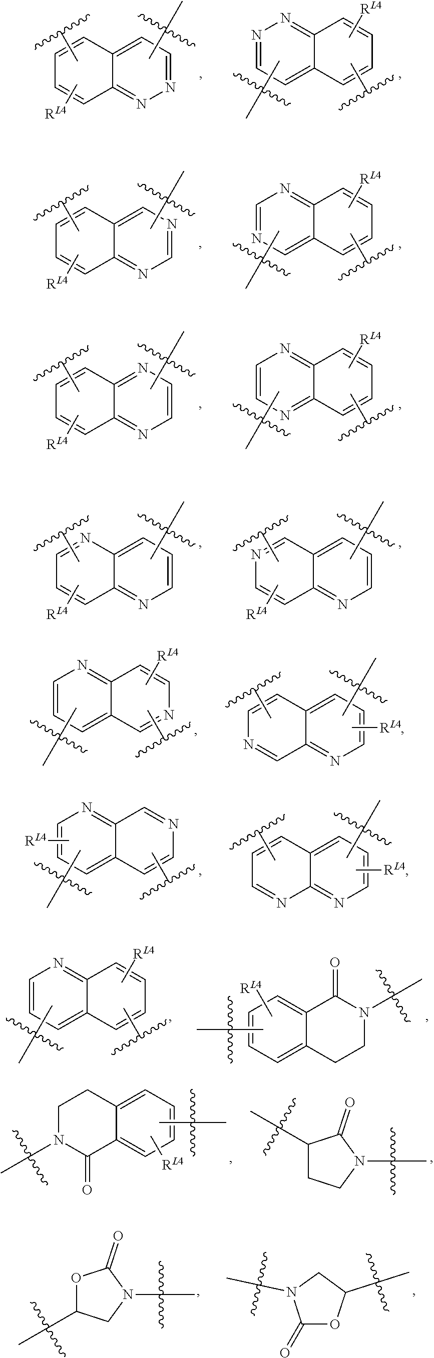 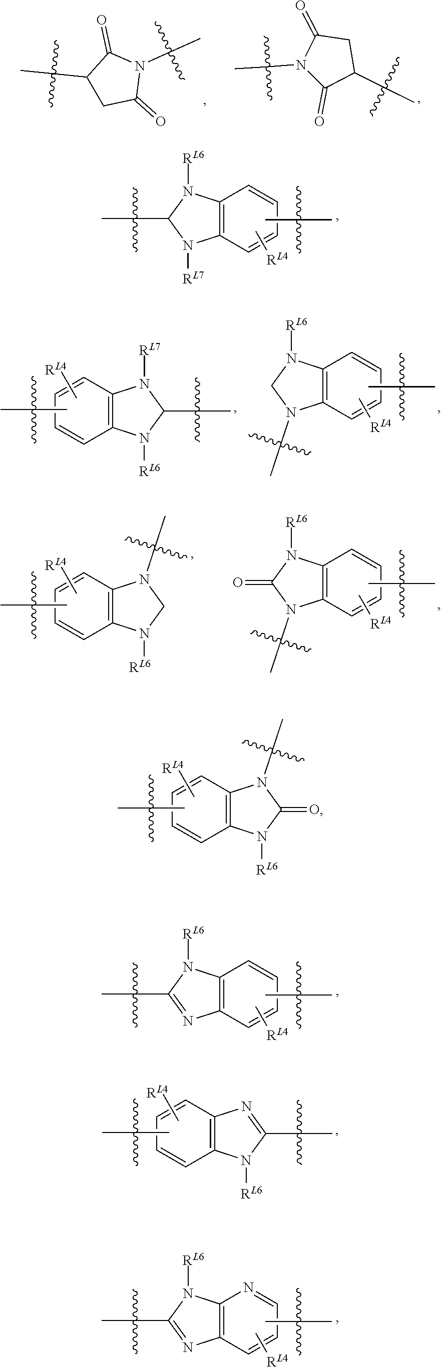 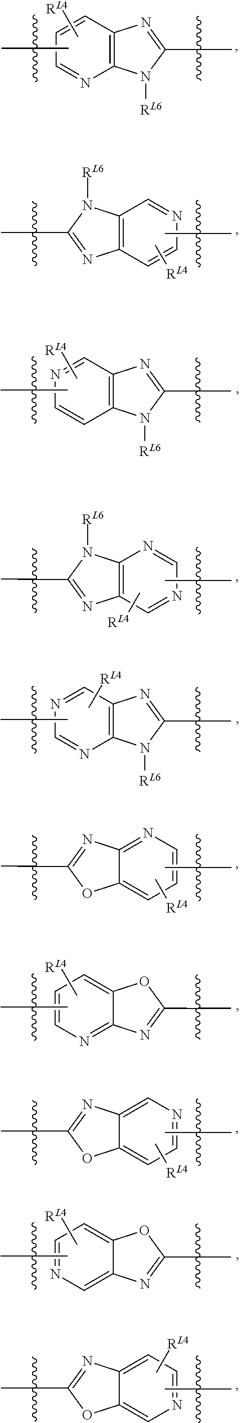 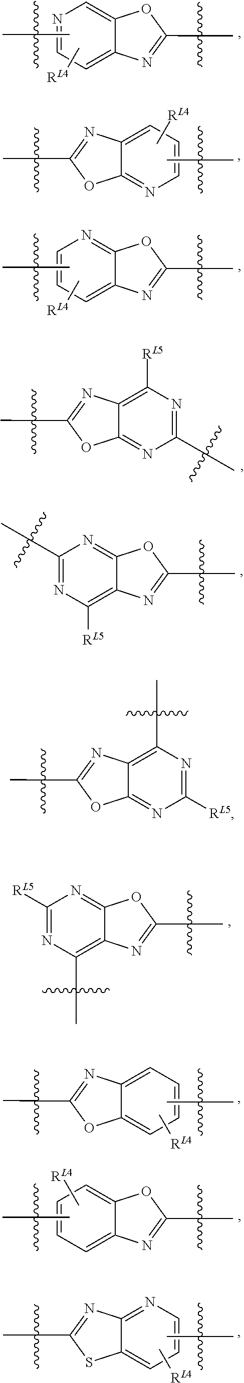 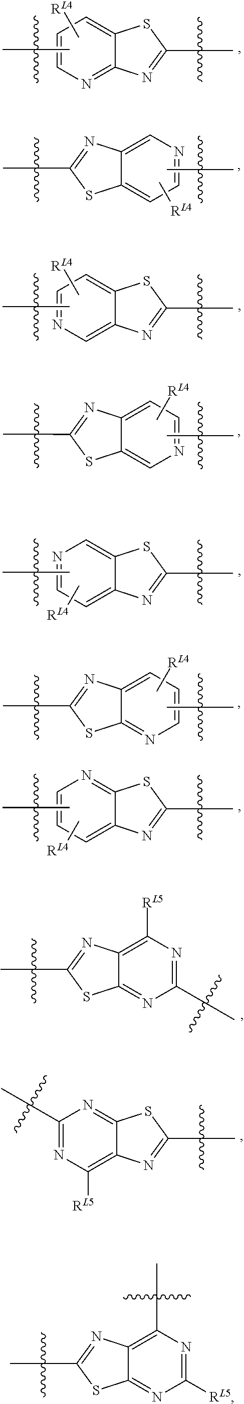 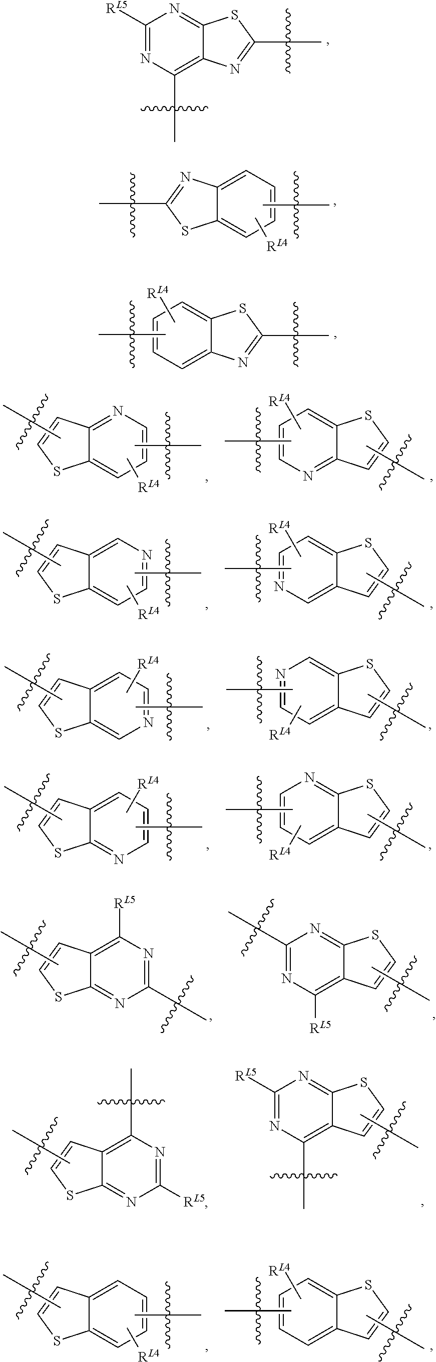 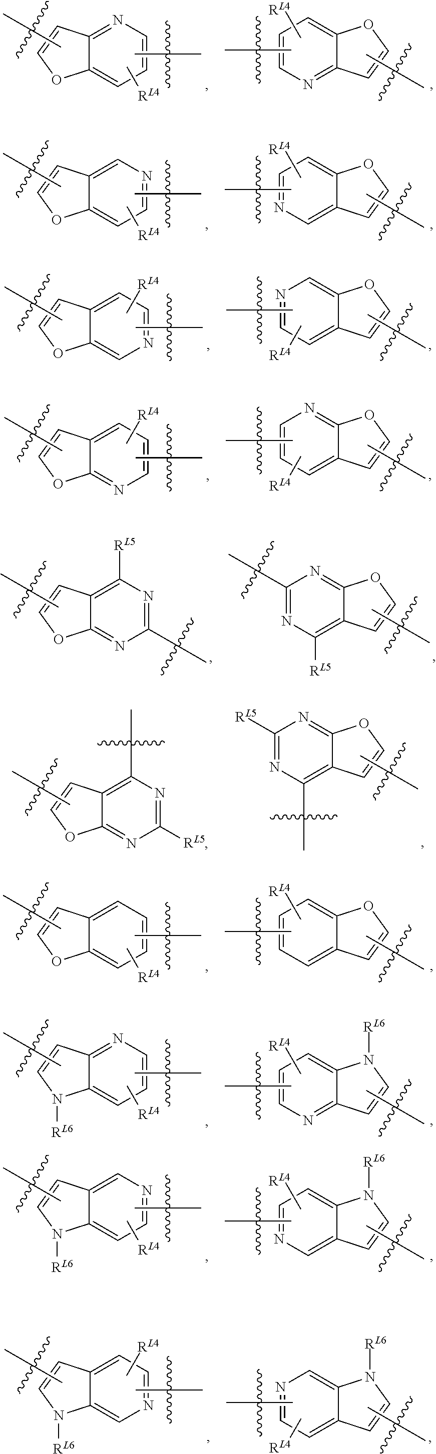 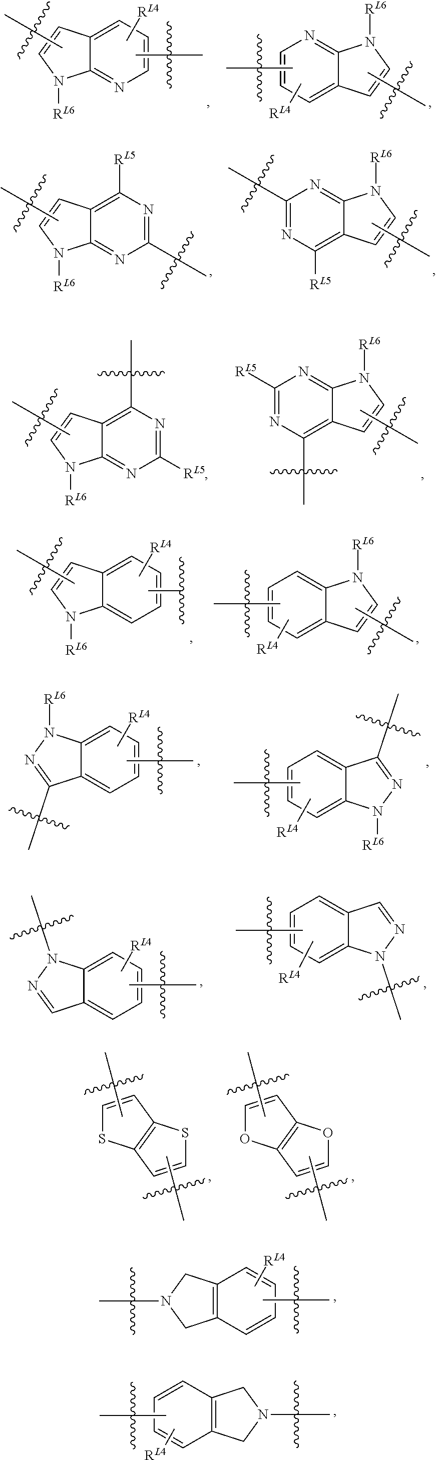 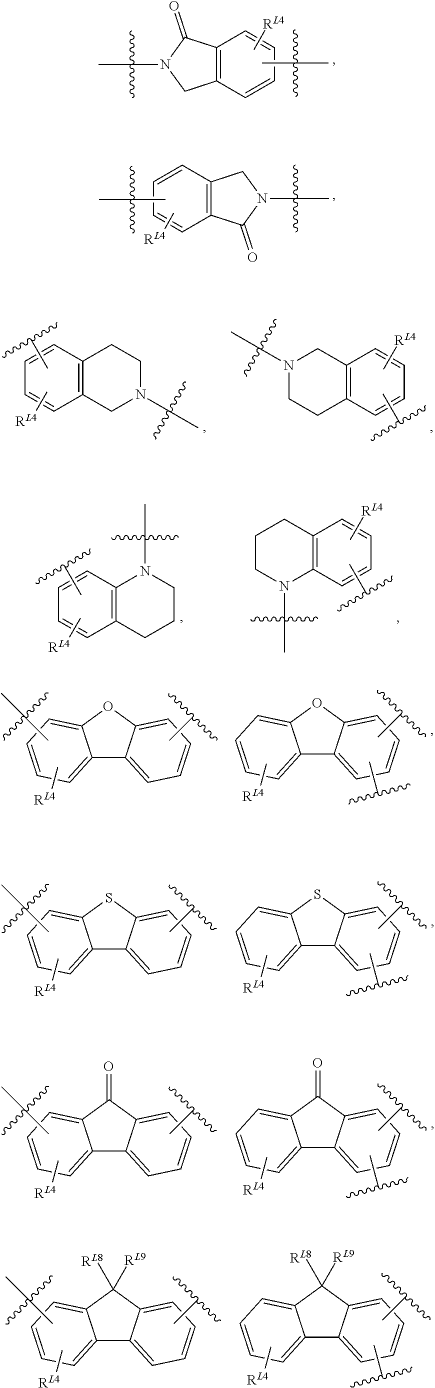 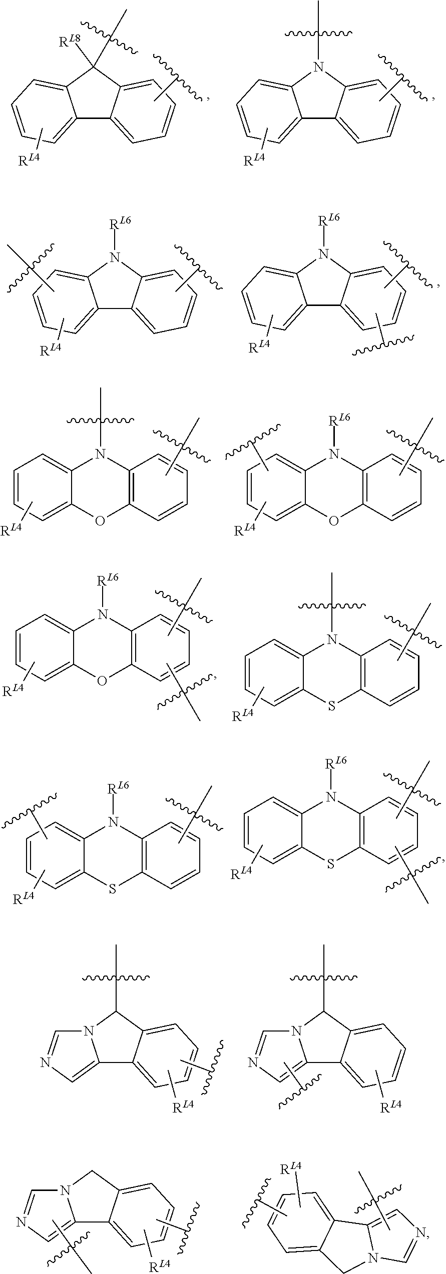 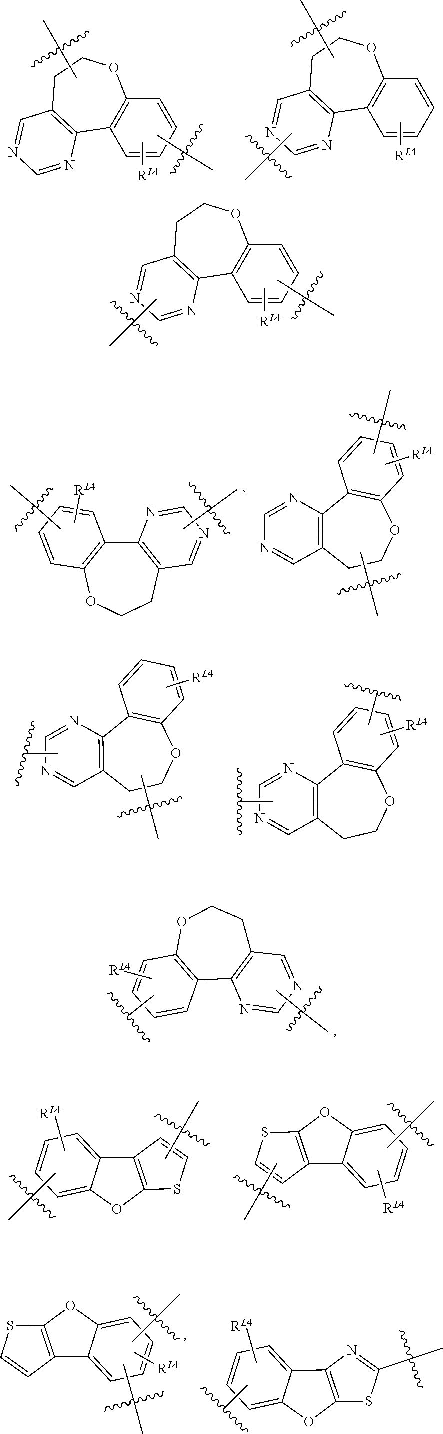 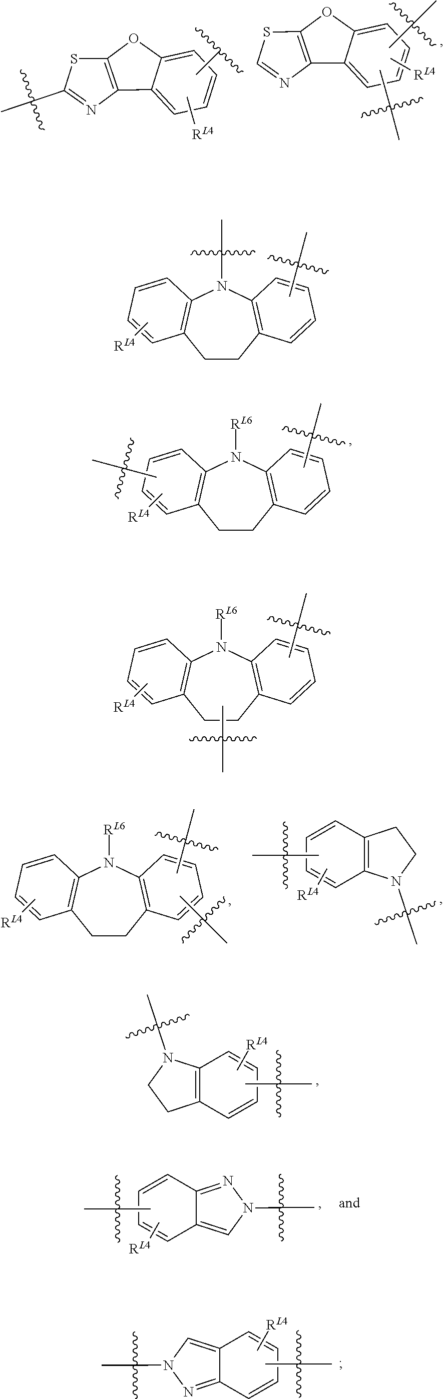
wherein R^{L4} is one or more independent substituents of a ring and is selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), and three- to six-membered heterocycloalkyl;
R^{L5} is selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), and —CO(C₀₋₁₀ alkyl);
R^{L6} and R^{L7} are independently selected from: C₀₋₁₀ alkyl, C₃₋₆ cycloalkyl, heterocycloalkyl, and heteroaryl;
R^{L8} and R^{L9} are independently selected from: halogen, —CN, —NO₂, —CF₃, —OCF₃, C₀₋₁₀ alkyl, —O(C₀₋₁₀ alkyl), —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), and —CO(C₀₋₁₀ alkyl); or R^{L8} and R^{L9}, together with a carbon atom linked to both R^{L8} and R^{L9}, form substituted or unsubstituted cycloalkyl or heterocyclyl;
preferably, each -Cy- is independently selected from: 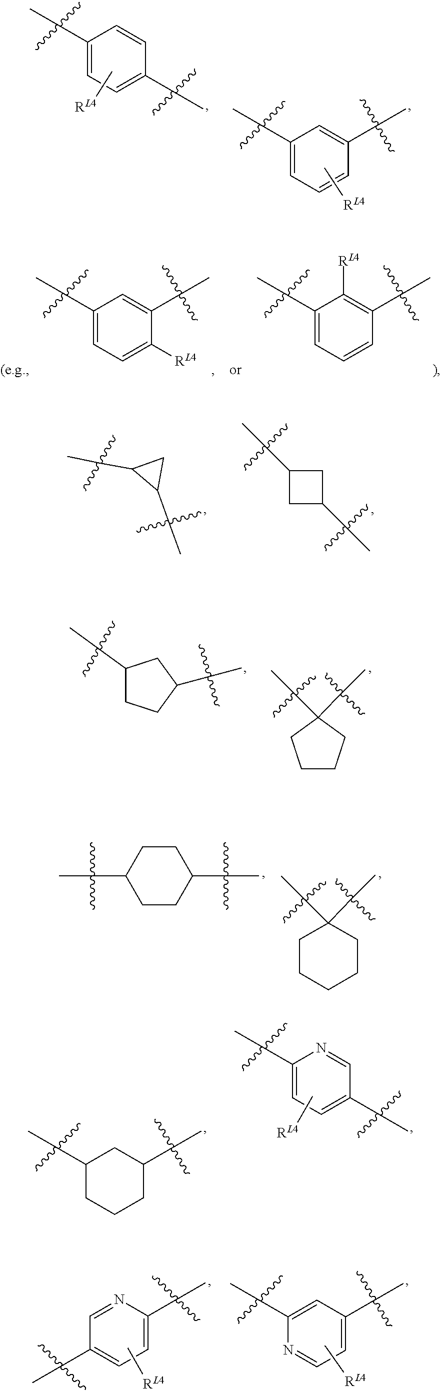 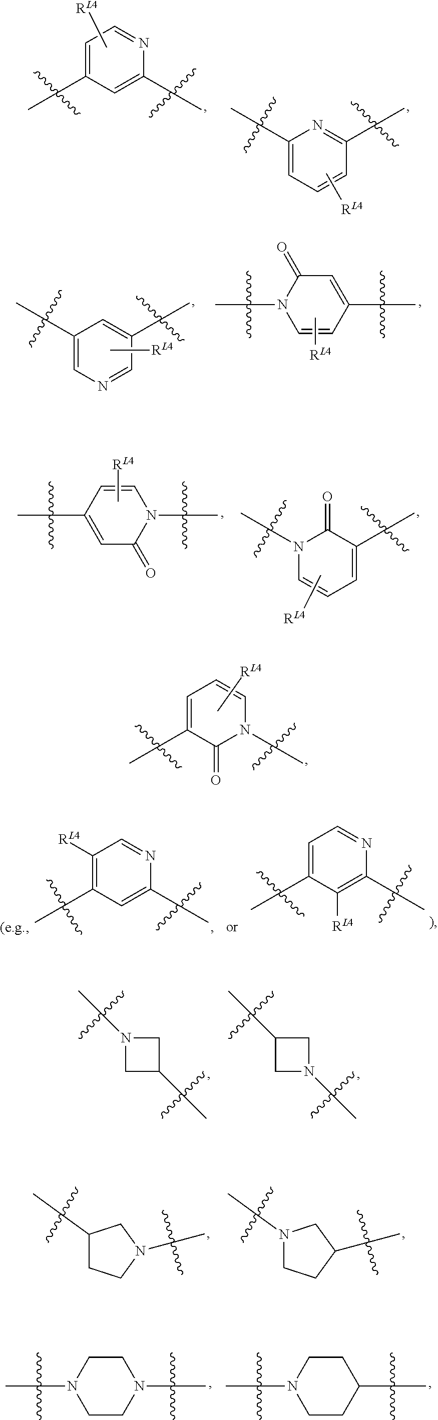 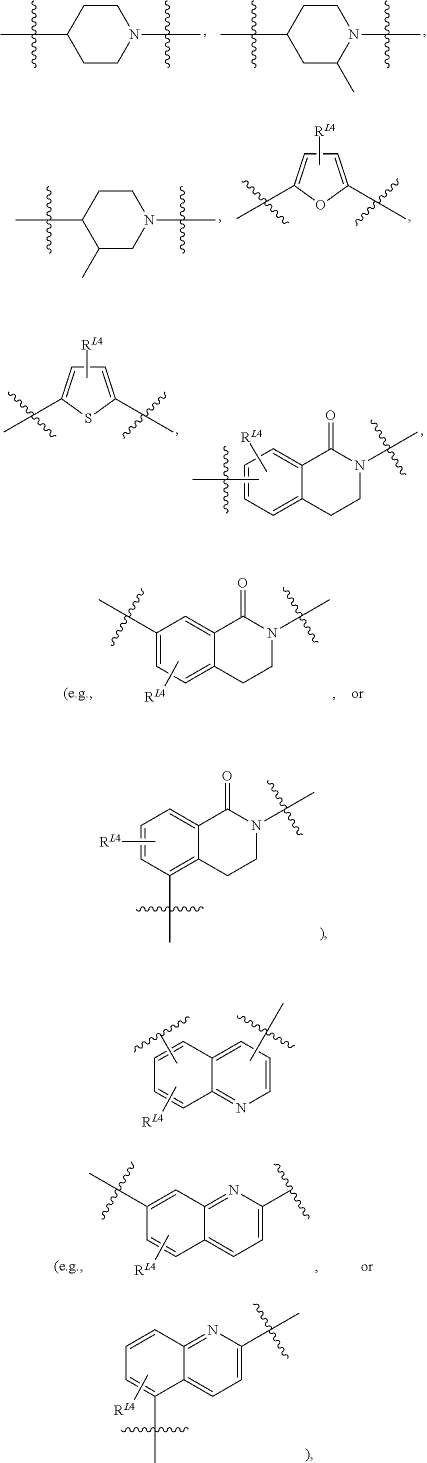 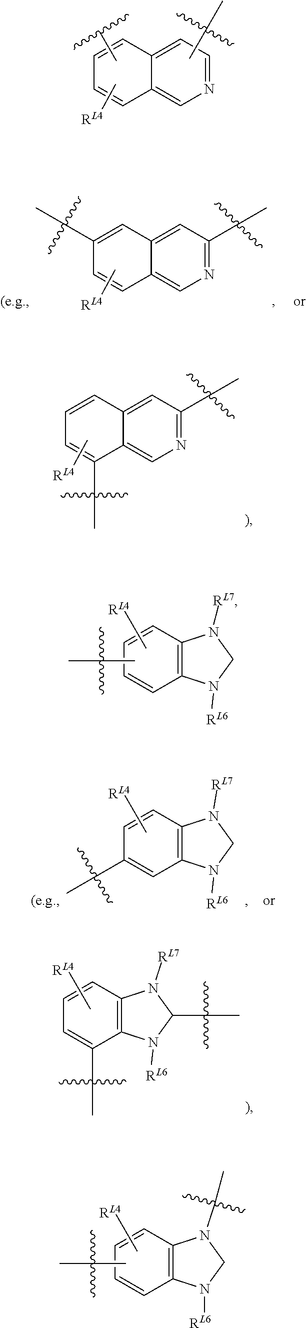 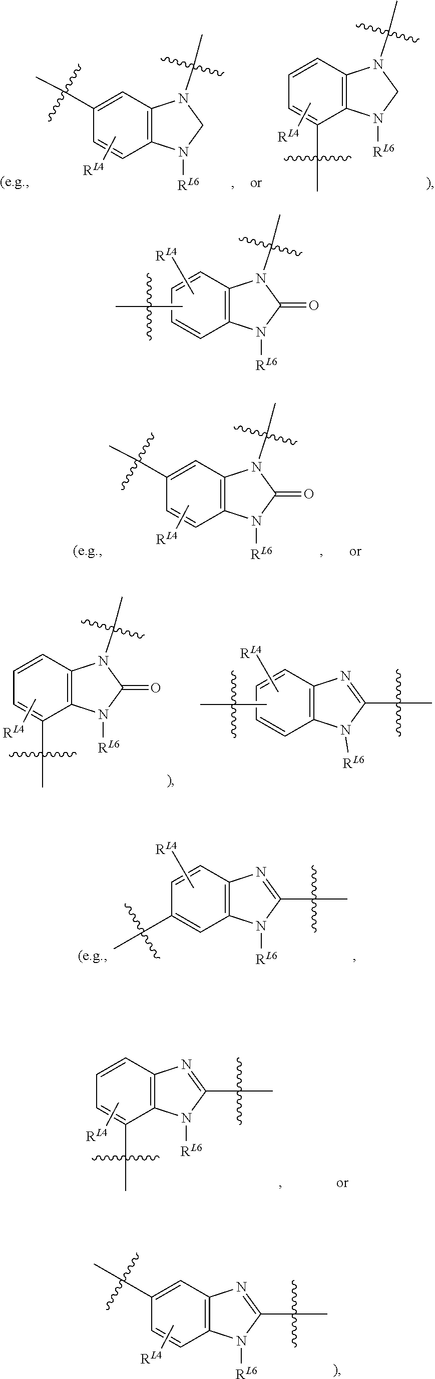 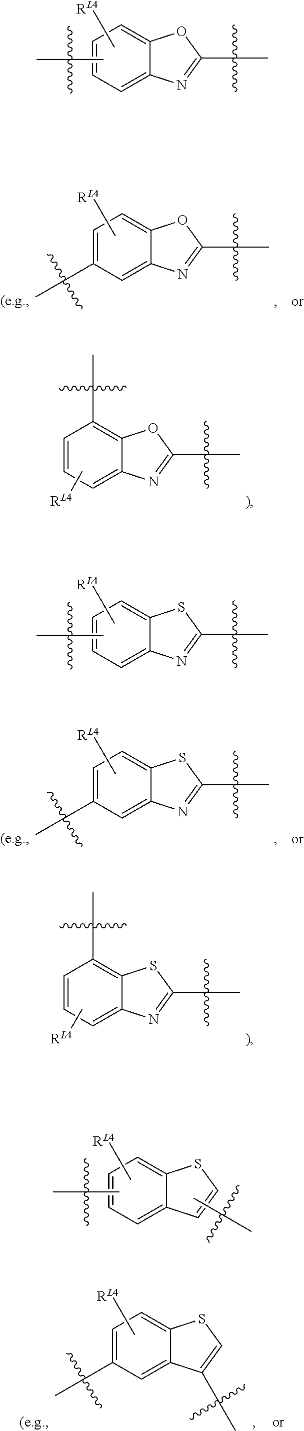 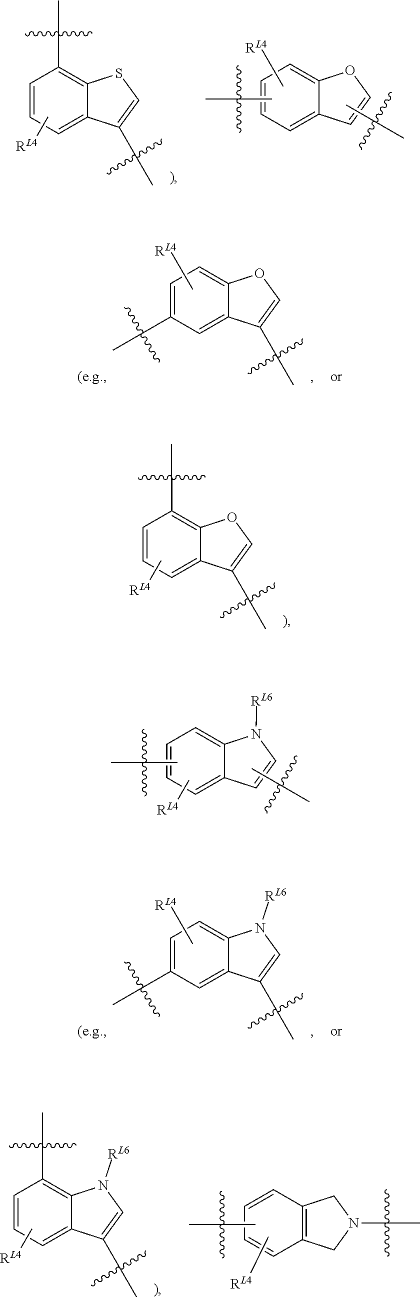 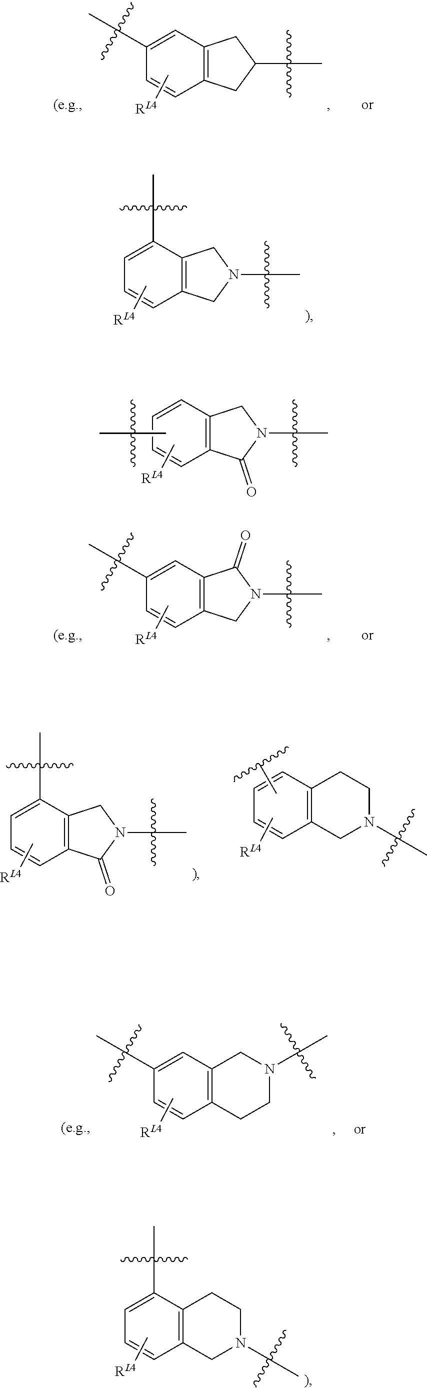 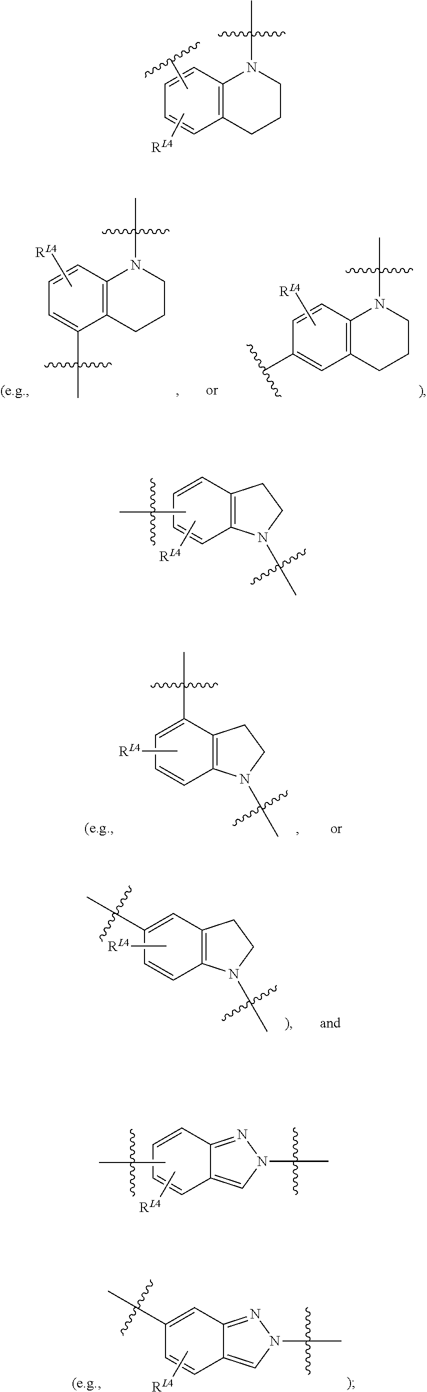
more preferably, each -Cy- is independently selected from:

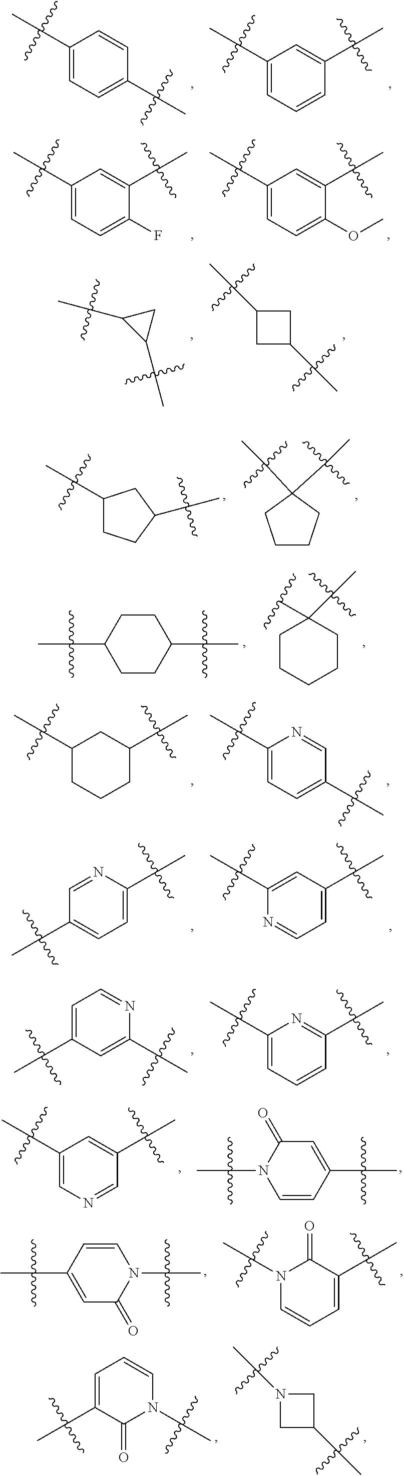

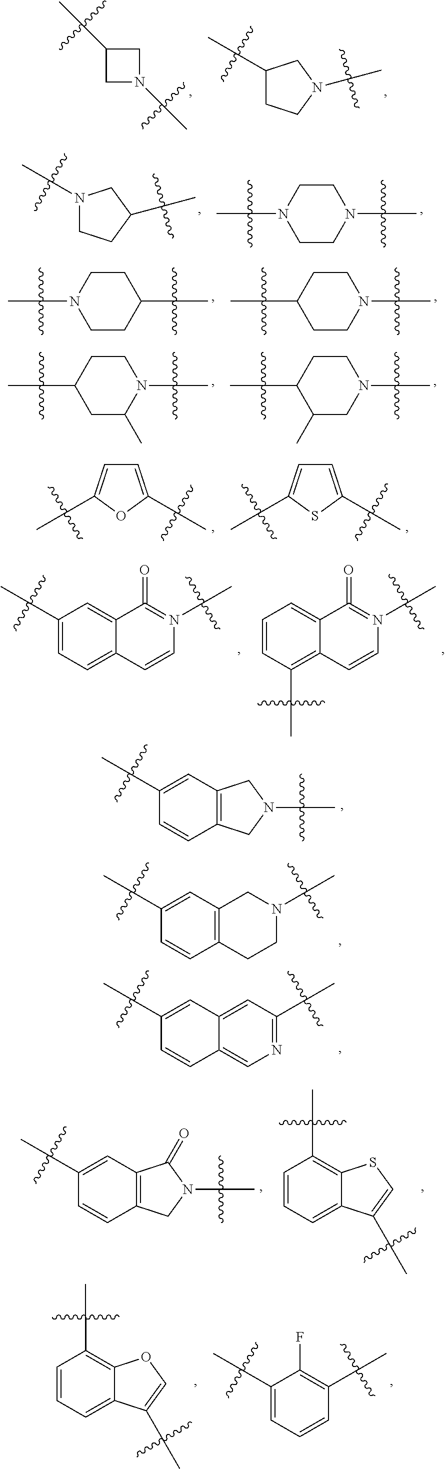

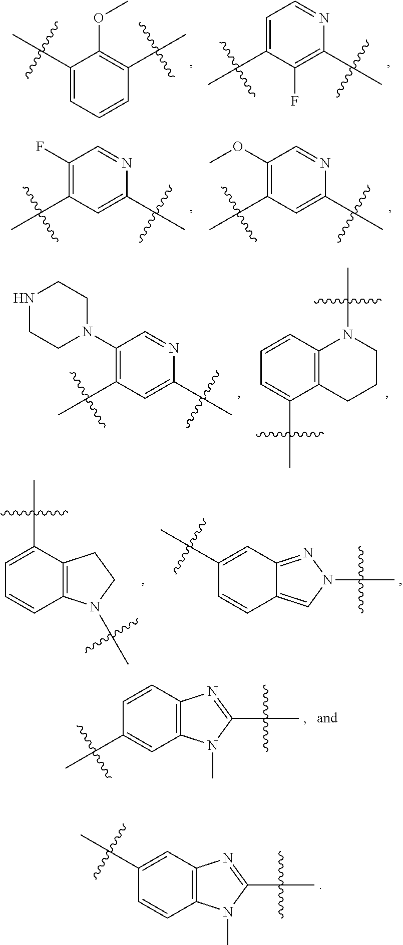

71. The compound according to any one of claims 57 to 70, wherein L₁ is —NR^{L1}—(C₀₋₆ alkylene)-, —O—(C₀₋₆ alkylene)-, —S—(C₀₋₆ alkylene)-, —(C₀₋₆ alkylene)-, 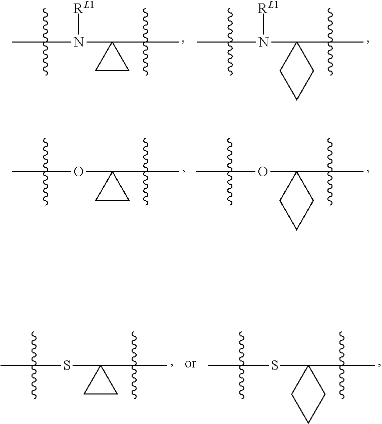
wherein R^{L1} is selected from: H, C₁₋₆ alkyl, wherein one or more H in the alkyl may be substituted with a substituent selected from: halogen (particularly F), —OH, —NH₂, C₁₋₆ alkoxy (e.g., methoxy, or ethoxy), and C₁₋₆ alkylamino 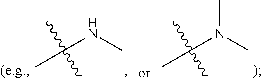
preferably, L₁ is selected from: —NH—, —NH—CH₂—, —NH—CH₂CH₂—, —NH—CH₂CH₂CH₂—, 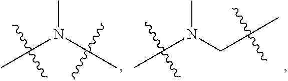
—O—, —O—CH₂—, —O—CH₂CH₂—, —O—CH₂CH₂CH₂—, —S—, —S—CH₂—, —S—CH₂CH₂—, —S—CH₂CH₂CH₂—, single bond, —CH₂—, —CH₂CH₂—, —CH₂CH₂CH₂—, and

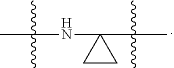 [SMILES: *NC1(*)CC1]

72. The compound according to any one of claims 57 to 71, wherein L₃ is —(C₀₋₆ alkylene)-NR^{L3}CO—, —(C₀₋₆ alkylene)-CONR^{L3}—, —(C₀₋₆ alkylene)-CO—, —(C₀₋₆ alkylene)-NH—, —(C₀₋₆ alkylene)-, 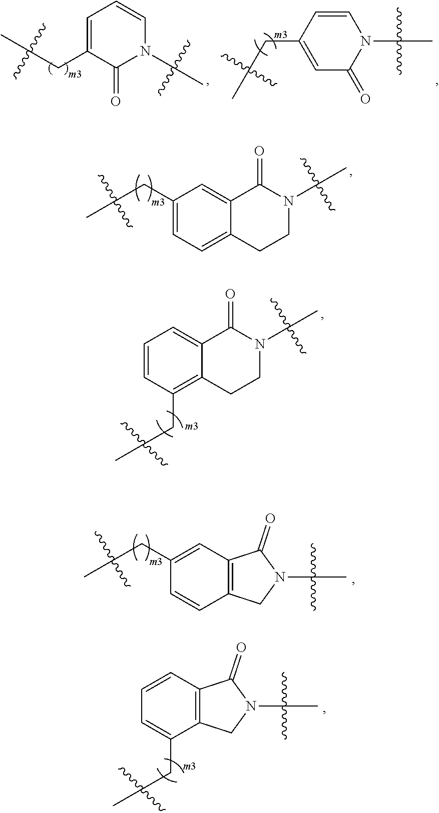
—NR^{L3}—(C₀₋₆ alkylene)-, —O—(C₀₋₆ alkylene)-, —S—(C₀₋₆ alkylene)-, —NR^{L3}CO—(C₀₋₆ alkylene)-, —CONR^{L3}—(C₀₋₆ alkylene)-, or —CO—(C₀₋₆ alkylene)-, wherein R^{L3} is selected from: H and C₁₋₆ alkyl, wherein one or more H in the alkyl may be substituted with a substituent selected from: halogen (particularly F), —OH, —NH₂, C₁₋₆ alkoxy (e.g., methoxy, or ethoxy), and C₁₋₆ alkylamino 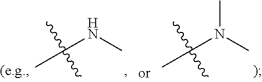
preferably, L₃ is selected from: —CONH—, —CH₂—CONH—, —CH₂CH₂—CONH—, —CH₂CH₂CH₂—CONH—, 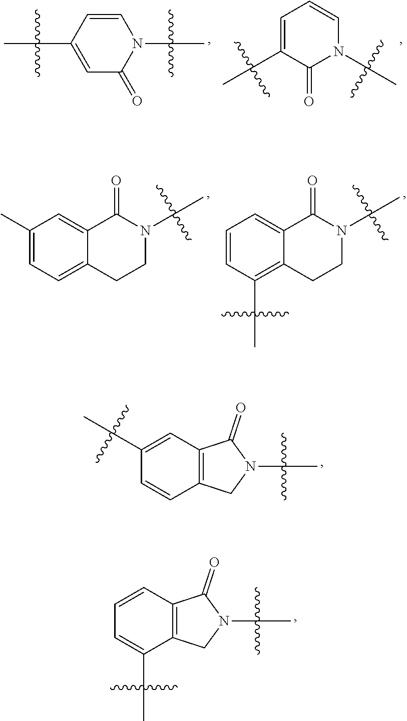 [SMILES: *c1ccn(*)c(=O)c1 ; *c1cccn(*)c1=O ; *N1CCc2ccc(C)cc2C1=O ; *c1cccc2c1CCN(*)C2=O ; *c1ccc2c(c1)C(=O)N(*)C2 ; *c1cccc2c1CN(*)C2=O]
—NH—, —CH₂—NH—, —CH₂CH₂—NH—, —CH₂CH₂CH₂—NH—, single bond, 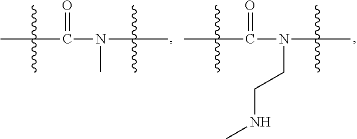
—NHCO—, 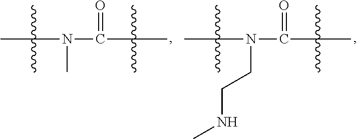
—NHCO—CH₂—, —CO—, —CO—CH₂—, —NH—CH₂—, 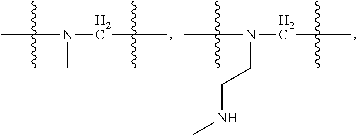
—O—CH₂—, and —S—CH₂—.

73. The compound according to any one of claims 57 to 72, wherein L₂ is selected from: C1-C20 linear alkylene, —(CH₂CH₂O)ₘ₂—, and -Cy-, wherein m2 is 0, 1, 2, 3, 4, or 5.

74. The compound according to any one of claims 57 to 69, wherein L is selected from:

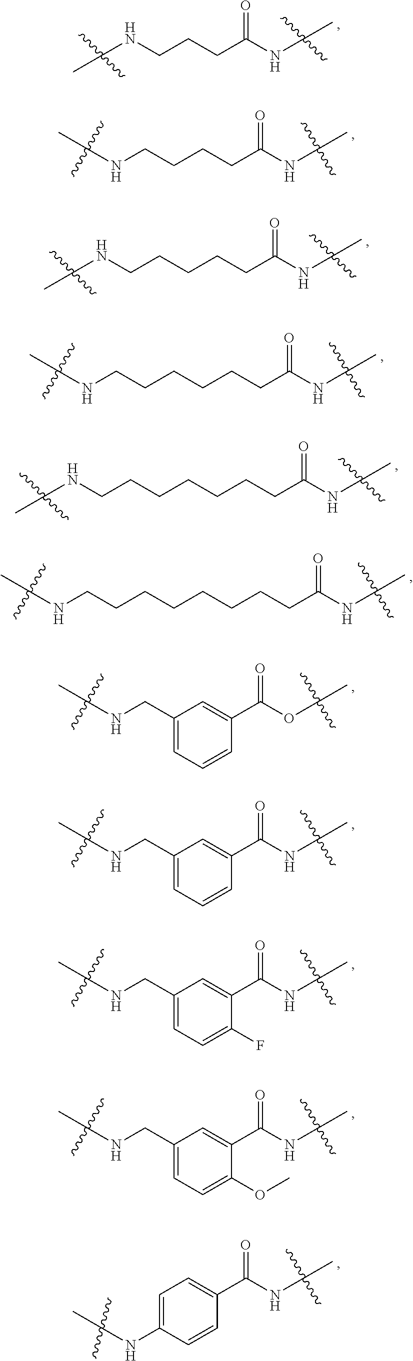

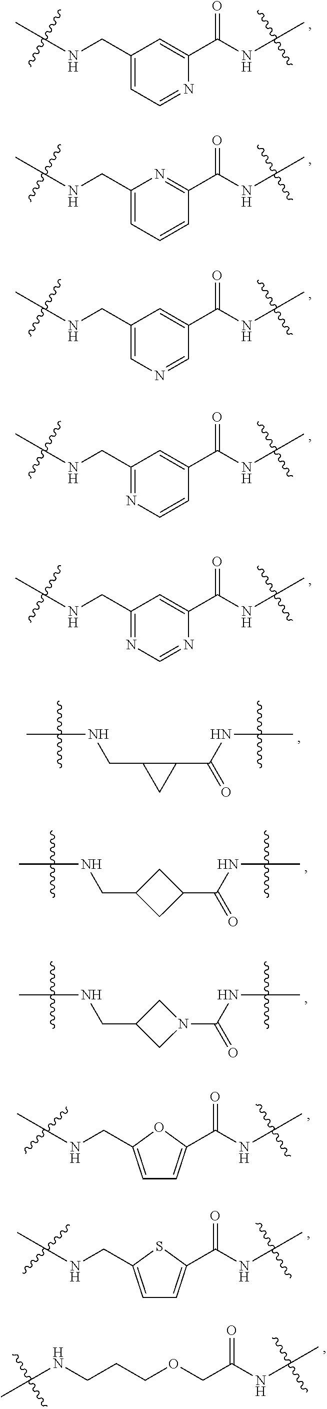

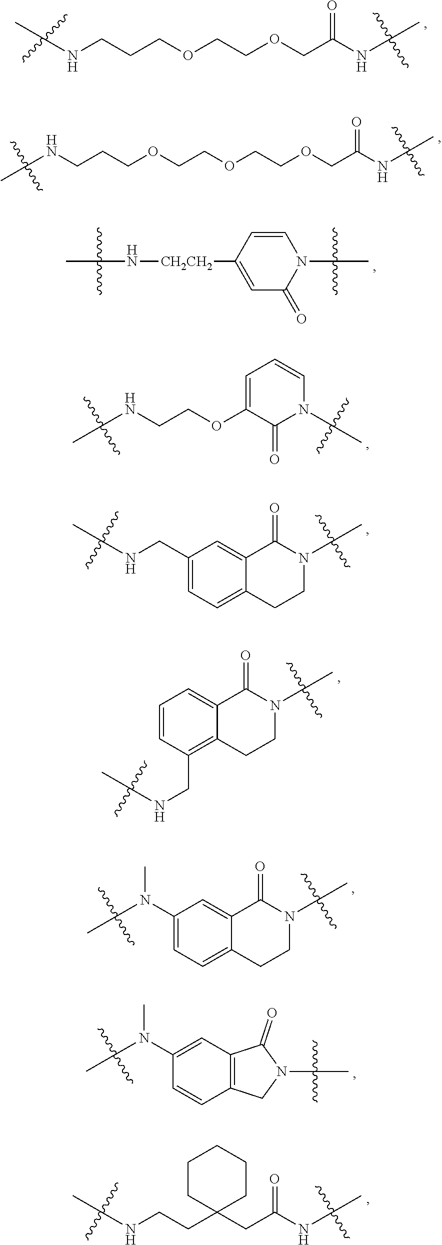

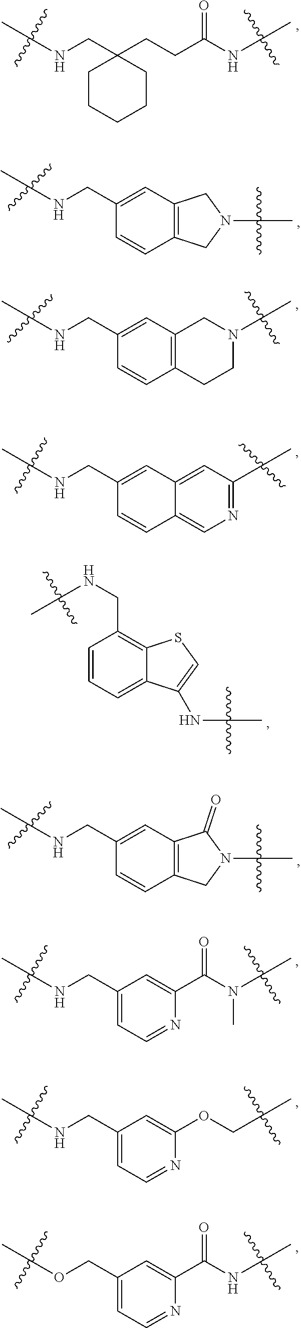

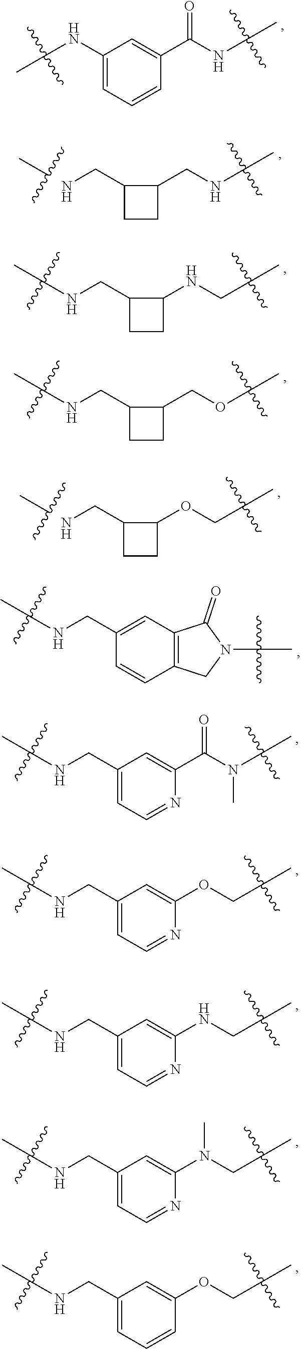

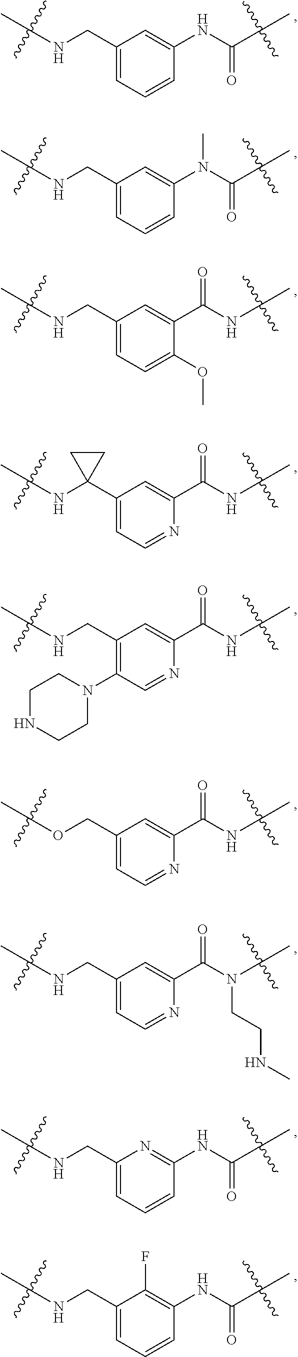

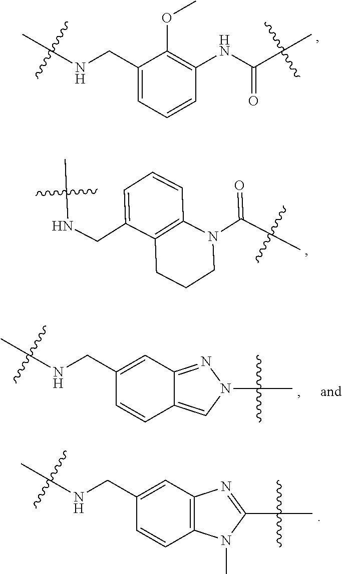 [SMILES: *NCc1cccc(NC(*)=O)c1OC ; *NCc1cccc2c1CCCN2C(*)=O ; *NCc1ccc2cn(*)nc2c1 ; *NCc1ccc2c(c1)nc(*)n2C]

75. The compound according to claim 57, selected from the following structures:

| ID | Structure |
|---|---|
| A1 | 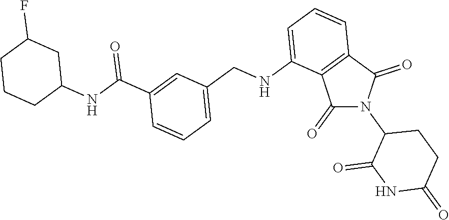 [SMILES: O=C1CCC(N2C(=O)c3cccc(NCc4cccc(C(=O)NC5CCCC(F)C5)c4)c3C2=O)C(=O)N1] |
| A2 | 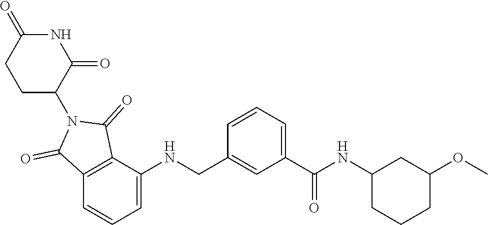 [SMILES: COC1CCCC(NC(=O)c2cccc(CNc3cccc4c3C(=O)N(C3CCC(=O)NC3=O)C4=O)c2)C1] |
| A3 | 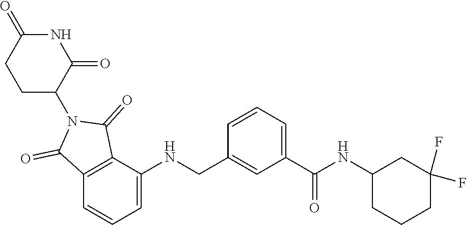 [SMILES: O=C1CCC(N2C(=O)c3cccc(NCc4cccc(C(=O)NC5CCCC(F)(F)C5)c4)c3C2=O)C(=O)N1] |
| A4 | 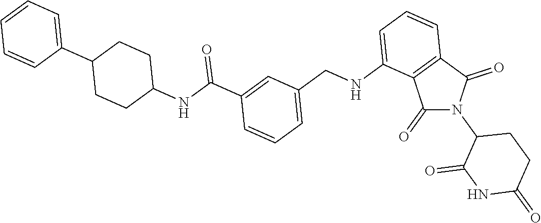 [SMILES: O=C1CCC(N2C(=O)c3cccc(NCc4cccc(C(=O)NC5CCC(c6ccccc6)CC5)c4)c3C2=O)C(=O)N1] |
| A5 | 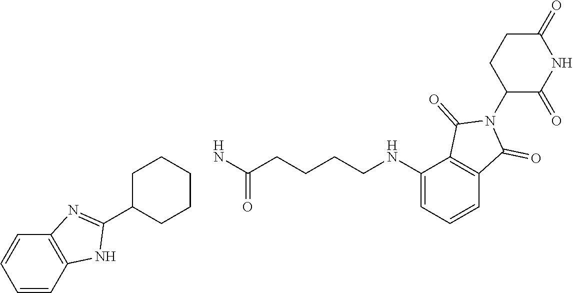 |
| A6 | 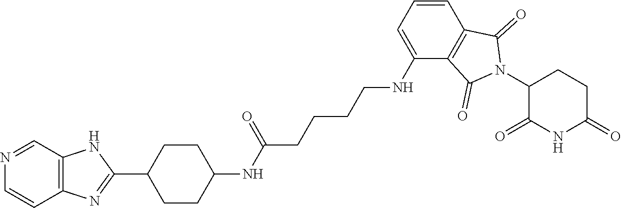 [SMILES: O=C1CCC(N2C(=O)c3cccc(NCCCCC(=O)NC4CCC(c5nc6ccncc6[nH]5)CC4)c3C2=O)C(=O)N1] |
| A7 | 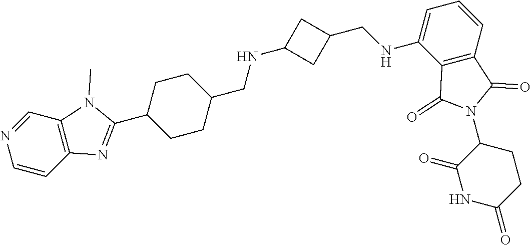 [SMILES: Cn1c(C2CCC(CNC3CC(CNc4cccc5c4C(=O)N(C4CCC(=O)NC4=O)C5=O)C3)CC2)nc2ccncc21] |
| A8 | 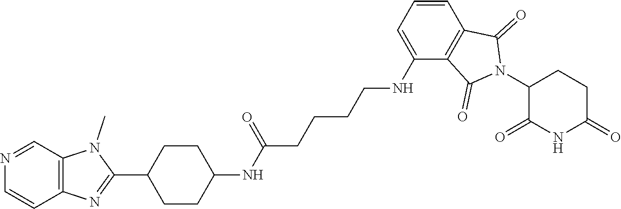 [SMILES: Cn1c(C2CCC(NC(=O)CCCCNc3cccc4c3C(=O)N(C3CCC(=O)NC3=O)C4=O)CC2)nc2ccncc21] |
| A9 | 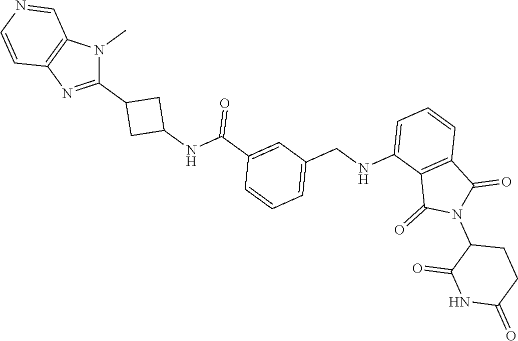 [SMILES: Cn1c(C2CC(NC(=O)c3cccc(CNc4cccc5c4C(=O)N(C4CCC(=O)NC4=O)C5=O)c3)C2)nc2ccncc21] |
| A10 | 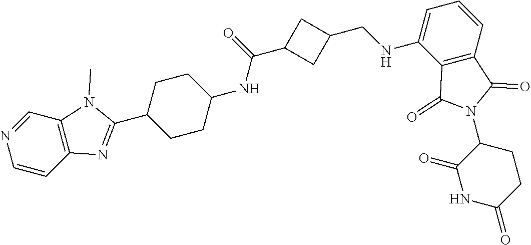 [SMILES: Cn1c(C2CCC(NC(=O)C3CC(CNc4cccc5c4C(=O)N(C4CCC(=O)NC4=O)C5=O)C3)CC2)nc2ccncc21] |
| A11 | 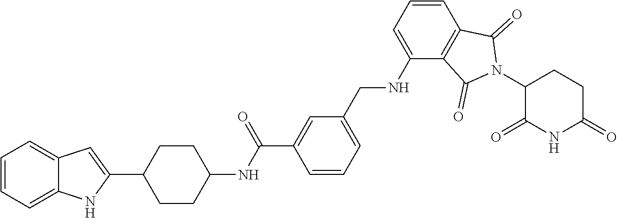 [SMILES: O=C1CCC(N2C(=O)c3cccc(NCc4cccc(C(=O)NC5CCC(c6cc7ccccc7[nH]6)CC5)c4)c3C2=O)C(=O)N1] |
| A12 | 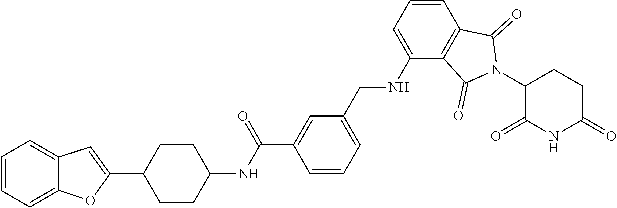 [SMILES: O=C1CCC(N2C(=O)c3cccc(NCc4cccc(C(=O)NC5CCC(c6cc7ccccc7o6)CC5)c4)c3C2=O)C(=O)N1] |
| A13 | 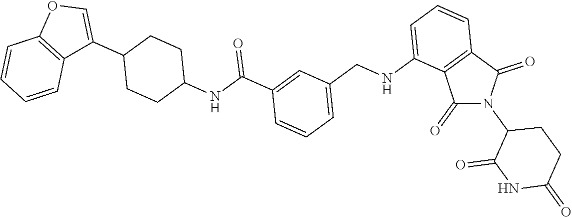 [SMILES: O=C1CCC(N2C(=O)c3cccc(NCc4cccc(C(=O)NC5CCC(c6coc7ccccc67)CC5)c4)c3C2=O)C(=O)N1] |
| A14 | 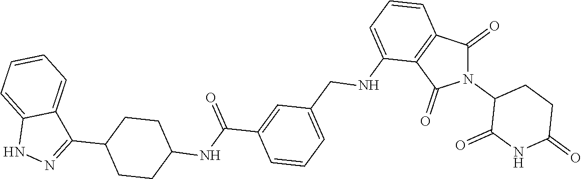 [SMILES: O=C1CCC(N2C(=O)c3cccc(NCc4cccc(C(=O)NC5CCC(c6n[nH]c7ccccc67)CC5)c4)c3C2=O)C(=O)N1] |
| A15 | 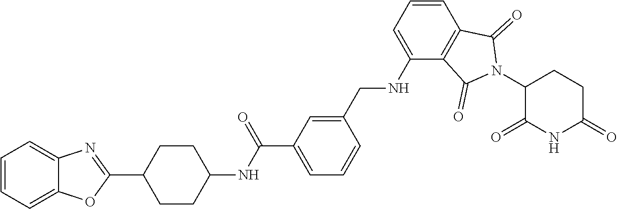 [SMILES: O=C1CCC(N2C(=O)c3cccc(NCc4cccc(C(=O)NC5CCC(c6nc7ccccc7o6)CC5)c4)c3C2=O)C(=O)N1] |
| A16 | 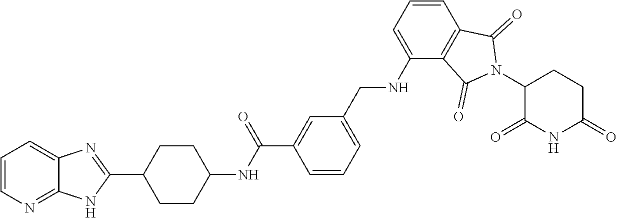 [SMILES: O=C1CCC(N2C(=O)c3cccc(NCc4cccc(C(=O)NC5CCC(c6nc7cccnc7[nH]6)CC5)c4)c3C2=O)C(=O)N1] |
| A17 | 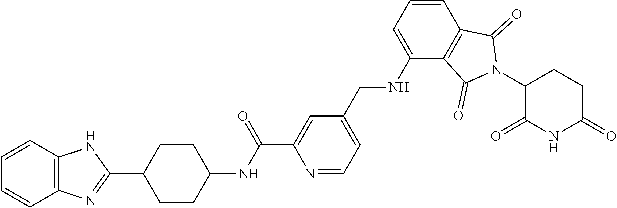 [SMILES: O=C1CCC(N2C(=O)c3cccc(NCc4ccnc(C(=O)NC5CCC(c6nc7ccccc7[nH]6)CC5)c4)c3C2=O)C(=O)N1] |
| A18 | 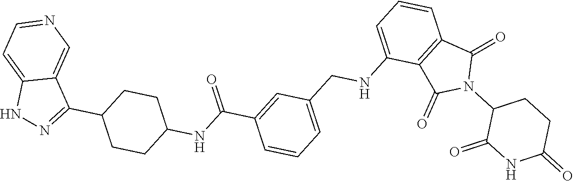 [SMILES: O=C1CCC(N2C(=O)c3cccc(NCc4cccc(C(=O)NC5CCC(c6n[nH]c7ccncc67)CC5)c4)c3C2=O)C(=O)N1] |
| A19 | 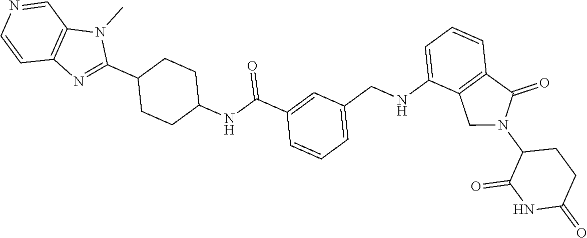 [SMILES: Cn1c(C2CCC(NC(=O)c3cccc(CNc4cccc5c4CN(C4CCC(=O)NC4=O)C5=O)c3)CC2)nc2ccncc21] |
| A20 | 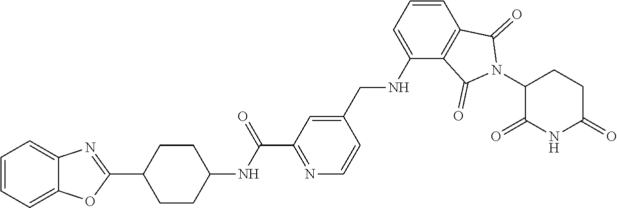 [SMILES: O=C1CCC(N2C(=O)c3cccc(NCc4ccnc(C(=O)NC5CCC(c6nc7ccccc7o6)CC5)c4)c3C2=O)C(=O)N1] |
| A21 | 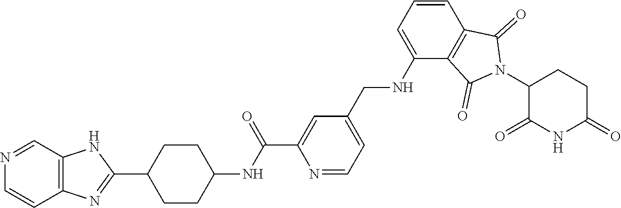 [SMILES: O=C1CCC(N2C(=O)c3cccc(NCc4ccnc(C(=O)NC5CCC(c6nc7ccncc7[nH]6)CC5)c4)c3C2=O)C(=O)N1] |
| A22 | 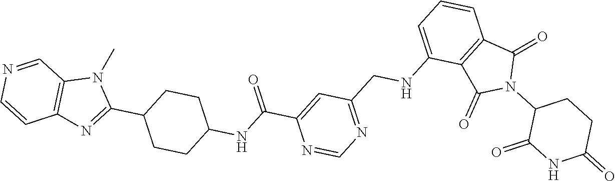 [SMILES: Cn1c(C2CCC(NC(=O)c3cc(CNc4cccc5c4C(=O)N(C4CCC(=O)NC4=O)C5=O)ncn3)CC2)nc2ccncc21] |
| A23 | 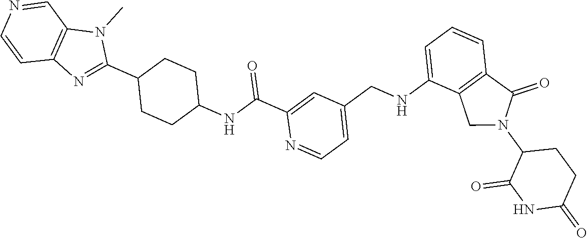 [SMILES: Cn1c(C2CCC(NC(=O)c3cc(CNc4cccc5c4CN(C4CCC(=O)NC4=O)C5=O)ccn3)CC2)nc2ccncc21] |
| A24 | 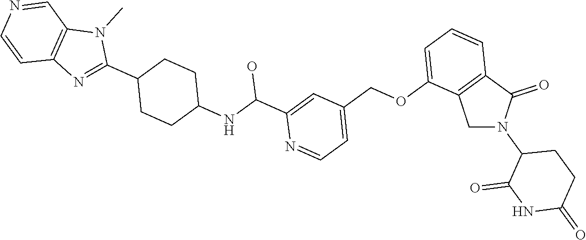 |
| A25 | 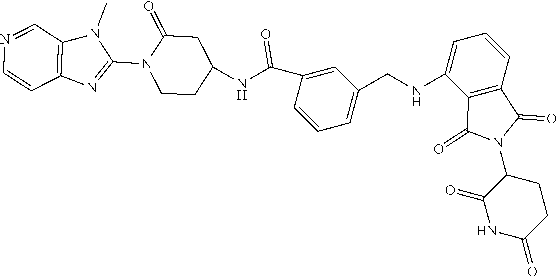 [SMILES: Cn1c(N2CCC(NC(=O)c3cccc(CNc4cccc5c4C(=O)N(C4CCC(=O)NC4=O)C5=O)c3)CC2=O)nc2ccncc21] |
| A26 | 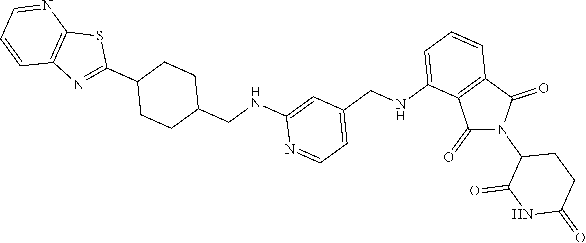 [SMILES: O=C1CCC(N2C(=O)c3cccc(NCc4ccnc(NCC5CCC(c6nc7cccnc7s6)CC5)c4)c3C2=O)C(=O)N1] |
| A27 | 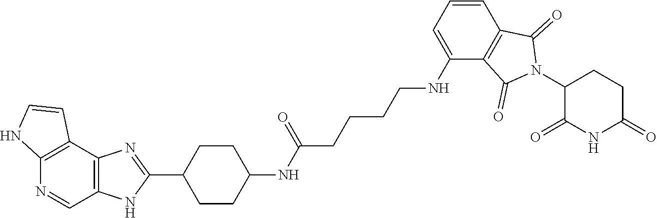 [SMILES: O=C1CCC(N2C(=O)c3cccc(NCCCCC(=O)NC4CCC(c5nc6c(cnc7[nH]ccc76)[nH]5)CC4)c3C2=O)C(=O)N1] |
| A28 | 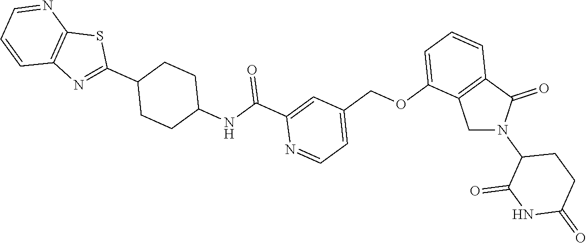 [SMILES: O=C1CCC(N2Cc3c(OCc4ccnc(C(=O)NC5CCC(c6nc7cccnc7s6)CC5)c4)cccc3C2=O)C(=O)N1] |
| A29 | 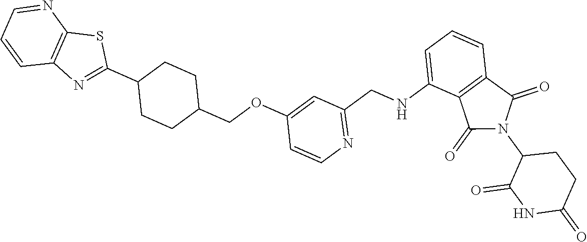 [SMILES: O=C1CCC(N2C(=O)c3cccc(NCc4cc(OCC5CCC(c6nc7cccnc7s6)CC5)ccn4)c3C2=O)C(=O)N1] |
| A30 | 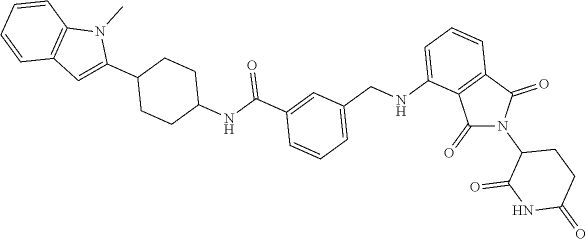 [SMILES: Cn1c(C2CCC(NC(=O)c3cccc(CNc4cccc5c4C(=O)N(C4CCC(=O)NC4=O)C5=O)c3)CC2)cc2ccccc21] |
| A31 | 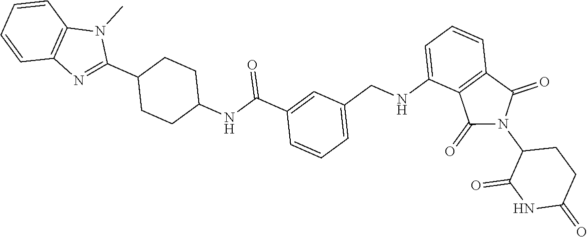 [SMILES: Cn1c(C2CCC(NC(=O)c3cccc(CNc4cccc5c4C(=O)N(C4CCC(=O)NC4=O)C5=O)c3)CC2)nc2ccccc21] |
| A32 | 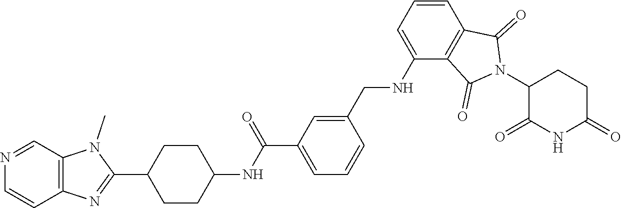 [SMILES: Cn1c(C2CCC(NC(=O)c3cccc(CNc4cccc5c4C(=O)N(C4CCC(=O)NC4=O)C5=O)c3)CC2)nc2ccncc21] |
| A33 | 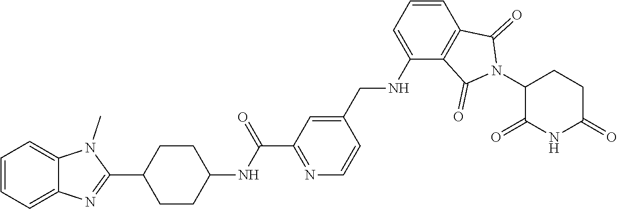 [SMILES: Cn1c(C2CCC(NC(=O)c3cc(CNc4cccc5c4C(=O)N(C4CCC(=O)NC4=O)C5=O)ccn3)CC2)nc2ccccc21] |
| A34 | 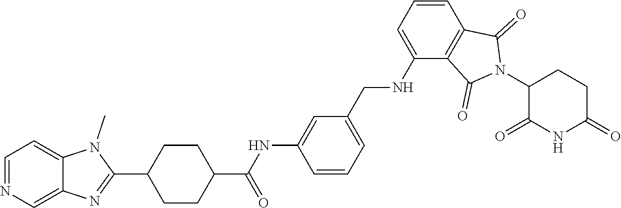 [SMILES: Cn1c(C2CCC(C(=O)Nc3cccc(CNc4cccc5c4C(=O)N(C4CCC(=O)NC4=O)C5=O)c3)CC2)nc2cnccc21] |
| A35 | 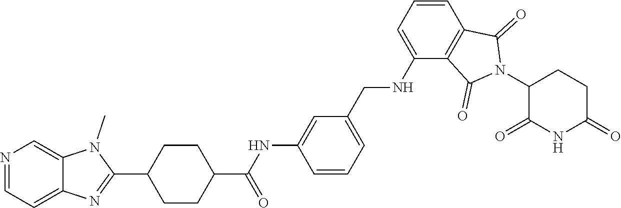 [SMILES: Cn1c(C2CCC(C(=O)Nc3cccc(CNc4cccc5c4C(=O)N(C4CCC(=O)NC4=O)C5=O)c3)CC2)nc2ccncc21] |
| A36 | 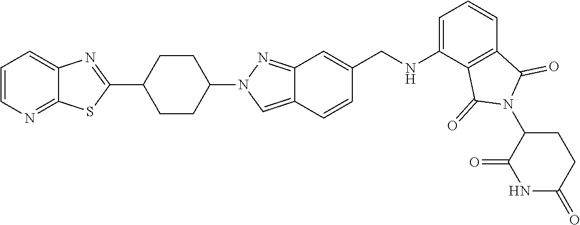 [SMILES: O=C1CCC(N2C(=O)c3cccc(NCc4ccc5cn(C6CCC(c7nc8cccnc8s7)CC6)nc5c4)c3C2=O)C(=O)N1] |
| A37 | 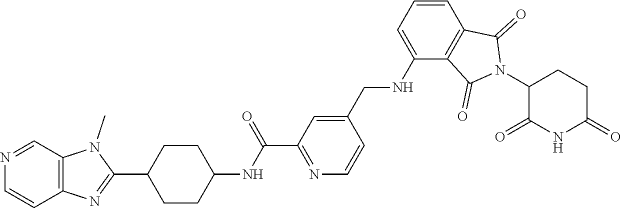 [SMILES: Cn1c(C2CCC(NC(=O)c3cc(CNc4cccc5c4C(=O)N(C4CCC(=O)NC4=O)C5=O)ccn3)CC2)nc2ccncc21] |
| A38 | 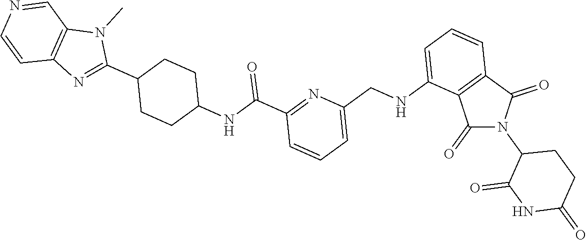 [SMILES: Cn1c(C2CCC(NC(=O)c3cccc(CNc4cccc5c4C(=O)N(C4CCC(=O)NC4=O)C5=O)n3)CC2)nc2ccncc21] |
| A39 | 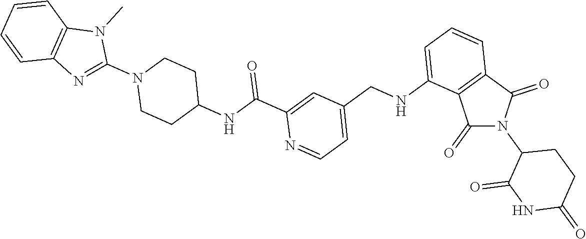 [SMILES: Cn1c(N2CCC(NC(=O)c3cc(CNc4cccc5c4C(=O)N(C4CCC(=O)NC4=O)C5=O)ccn3)CC2)nc2ccccc21] |
| A40 | 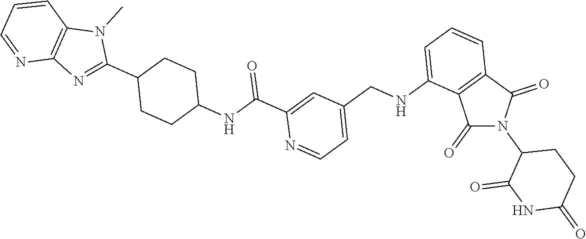 [SMILES: Cn1c(C2CCC(NC(=O)c3cc(CNc4cccc5c4C(=O)N(C4CCC(=O)NC4=O)C5=O)ccn3)CC2)nc2ncccc21] |
| A41 | 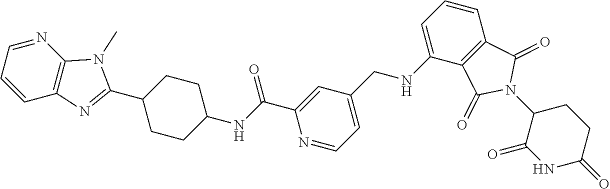 [SMILES: Cn1c(C2CCC(NC(=O)c3cc(CNc4cccc5c4C(=O)N(C4CCC(=O)NC4=O)C5=O)ccn3)CC2)nc2cccnc21] |
| A42 | 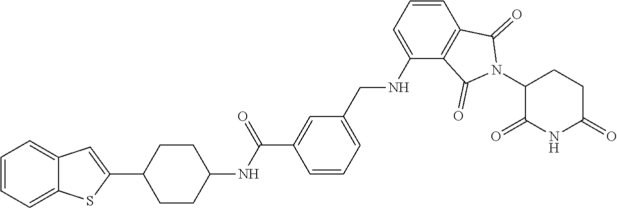 [SMILES: O=C1CCC(N2C(=O)c3cccc(NCc4cccc(C(=O)NC5CCC(c6cc7ccccc7s6)CC5)c4)c3C2=O)C(=O)N1] |
| A43 | 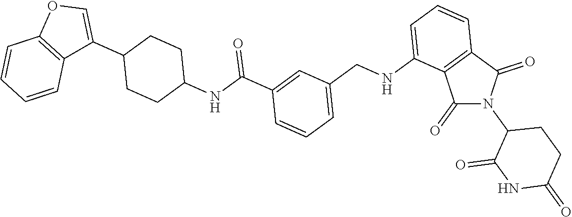 [SMILES: O=C1CCC(N2C(=O)c3cccc(NCc4cccc(C(=O)NC5CCC(c6coc7ccccc67)CC5)c4)c3C2=O)C(=O)N1] |
| A44 | 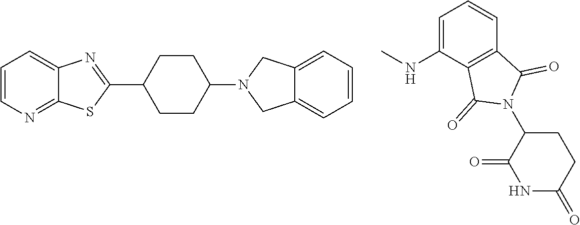 |
| A45 | 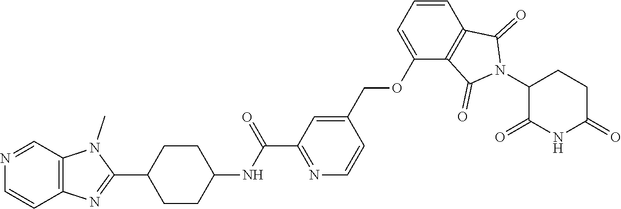 [SMILES: Cn1c(C2CCC(NC(=O)c3cc(COc4cccc5c4C(=O)N(C4CCC(=O)NC4=O)C5=O)ccn3)CC2)nc2ccncc21] |
| A46 | 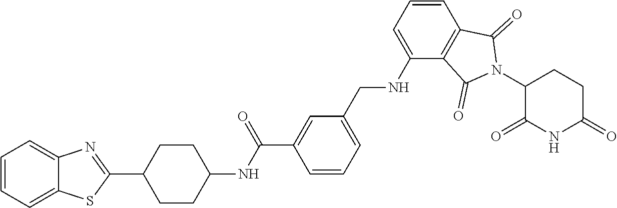 [SMILES: O=C1CCC(N2C(=O)c3cccc(NCc4cccc(C(=O)NC5CCC(c6nc7ccccc7s6)CC5)c4)c3C2=O)C(=O)N1] |
| A47 | 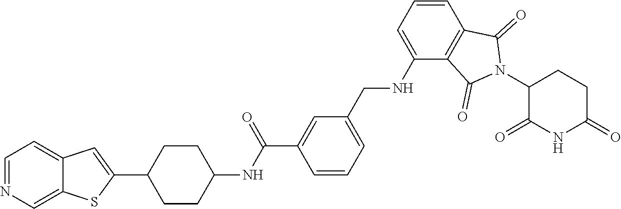 [SMILES: O=C1CCC(N2C(=O)c3cccc(NCc4cccc(C(=O)NC5CCC(c6cc7ccncc7s6)CC5)c4)c3C2=O)C(=O)N1] |
| A48 | 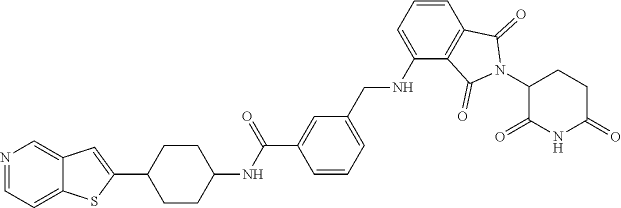 [SMILES: O=C1CCC(N2C(=O)c3cccc(NCc4cccc(C(=O)NC5CCC(c6cc7cnccc7s6)CC5)c4)c3C2=O)C(=O)N1] |
| A49 | 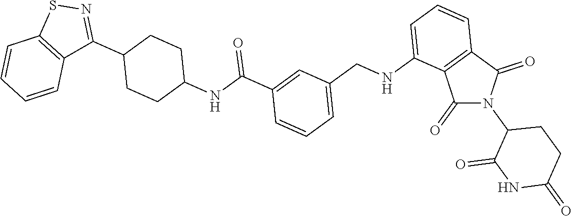 [SMILES: O=C1CCC(N2C(=O)c3cccc(NCc4cccc(C(=O)NC5CCC(c6nsc7ccccc67)CC5)c4)c3C2=O)C(=O)N1] |
| A50 | 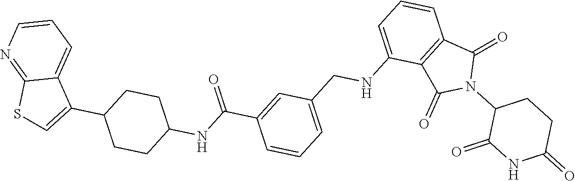 [SMILES: O=C1CCC(N2C(=O)c3cccc(NCc4cccc(C(=O)NC5CCC(c6csc7ncccc67)CC5)c4)c3C2=O)C(=O)N1] |
| A51 | 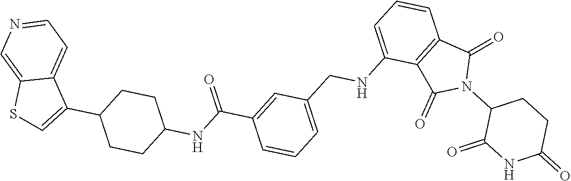 [SMILES: O=C1CCC(N2C(=O)c3cccc(NCc4cccc(C(=O)NC5CCC(c6csc7cnccc67)CC5)c4)c3C2=O)C(=O)N1] |
| A52 | 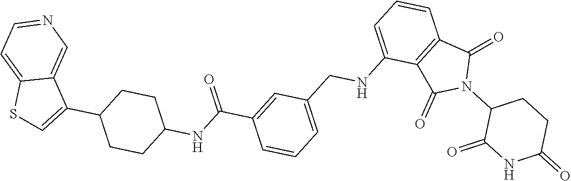 [SMILES: O=C1CCC(N2C(=O)c3cccc(NCc4cccc(C(=O)NC5CCC(c6csc7ccncc67)CC5)c4)c3C2=O)C(=O)N1] |
| A53 | 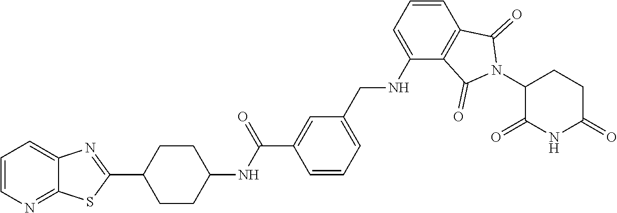 [SMILES: O=C1CCC(N2C(=O)c3cccc(NCc4cccc(C(=O)NC5CCC(c6nc7cccnc7s6)CC5)c4)c3C2=O)C(=O)N1] |
| A54 | 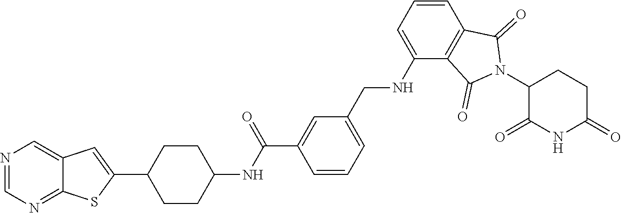 [SMILES: O=C1CCC(N2C(=O)c3cccc(NCc4cccc(C(=O)NC5CCC(c6cc7cncnc7s6)CC5)c4)c3C2=O)C(=O)N1] |
| A55 | 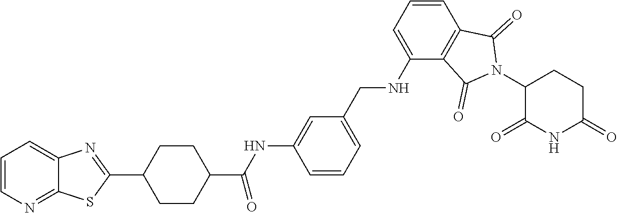 [SMILES: O=C1CCC(N2C(=O)c3cccc(NCc4cccc(NC(=O)C5CCC(c6nc7cccnc7s6)CC5)c4)c3C2=O)C(=O)N1] |
| A56 | 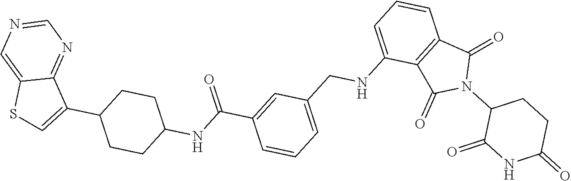 [SMILES: O=C1CCC(N2C(=O)c3cccc(NCc4cccc(C(=O)NC5CCC(c6csc7cncnc67)CC5)c4)c3C2=O)C(=O)N1] |
| A57 | 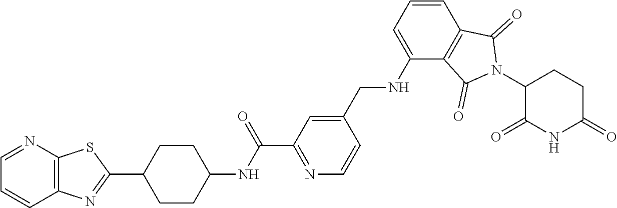 [SMILES: O=C1CCC(N2C(=O)c3cccc(NCc4ccnc(C(=O)NC5CCC(c6nc7cccnc7s6)CC5)c4)c3C2=O)C(=O)N1] |
| A58 | 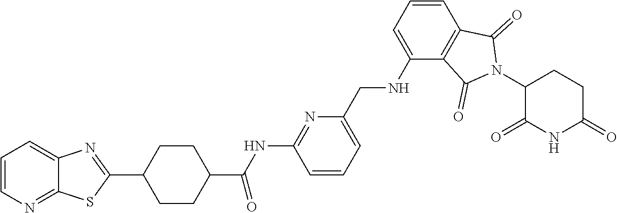 [SMILES: O=C1CCC(N2C(=O)c3cccc(NCc4cccc(NC(=O)C5CCC(c6nc7cccnc7s6)CC5)n4)c3C2=O)C(=O)N1] |
| A59 | 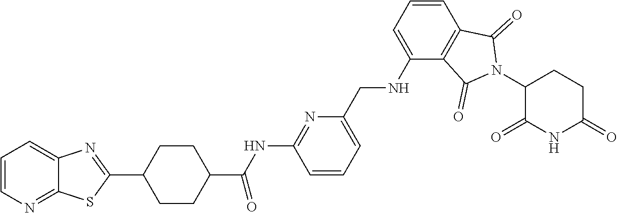 [SMILES: O=C1CCC(N2C(=O)c3cccc(NCc4cccc(NC(=O)C5CCC(c6nc7cccnc7s6)CC5)n4)c3C2=O)C(=O)N1] |
| A60 | 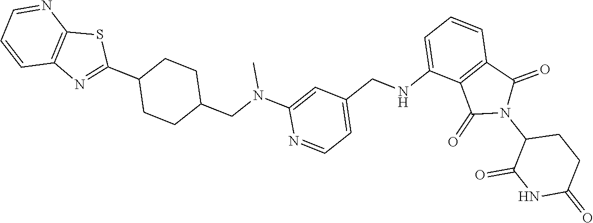 [SMILES: CN(CC1CCC(c2nc3cccnc3s2)CC1)c1cc(CNc2cccc3c2C(=O)N(C2CCC(=O)NC2=O)C3=O)ccn1] |
| A61 | 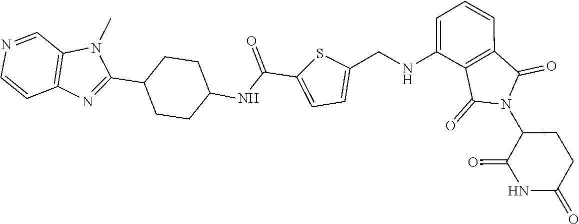 [SMILES: Cn1c(C2CCC(NC(=O)c3ccc(CNc4cccc5c4C(=O)N(C4CCC(=O)NC4=O)C5=O)s3)CC2)nc2ccncc21] |
| A62 | 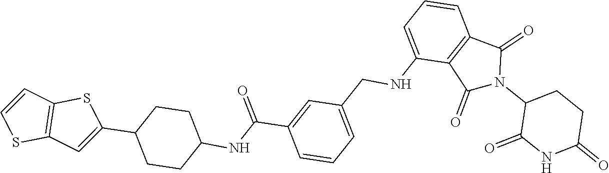 [SMILES: O=C1CCC(N2C(=O)c3cccc(NCc4cccc(C(=O)NC5CCC(c6cc7sccc7s6)CC5)c4)c3C2=O)C(=O)N1] |
| A63 | 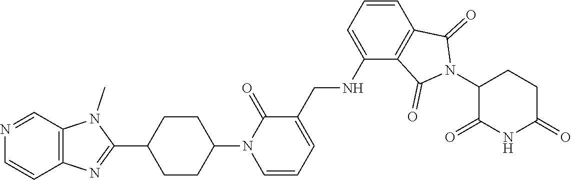 [SMILES: Cn1c(C2CCC(n3cccc(CNc4cccc5c4C(=O)N(C4CCC(=O)NC4=O)C5=O)c3=O)CC2)nc2ccncc21] |
| A64 | 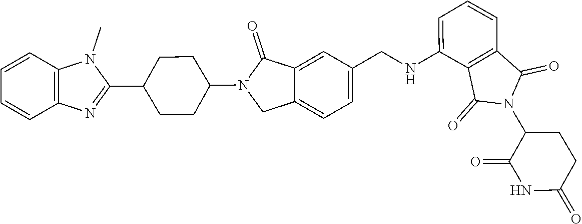 [SMILES: Cn1c(C2CCC(N3Cc4ccc(CNc5cccc6c5C(=O)N(C5CCC(=O)NC5=O)C6=O)cc4C3=O)CC2)nc2ccccc21] |
| A65 | 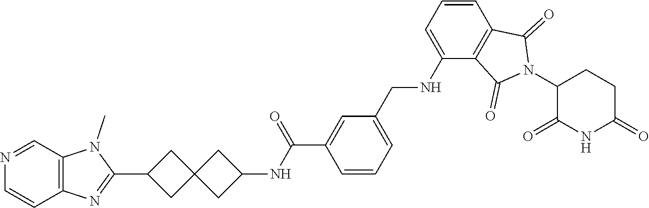 [SMILES: Cn1c(C2CC3(CC(NC(=O)c4cccc(CNc5cccc6c5C(=O)N(C5CCC(=O)NC5=O)C6=O)c4)C3)C2)nc2ccncc21] |
| A66 | 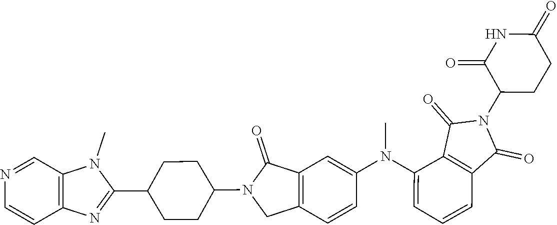 [SMILES: CN(c1ccc2c(c1)C(=O)N(C1CCC(c3nc4ccncc4n3C)CC1)C2)c1cccc2c1C(=O)N(C1CCC(=O)NC1=O)C2=O] |
| A67 | 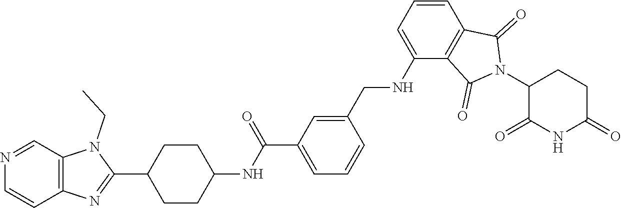 [SMILES: CCn1c(C2CCC(NC(=O)c3cccc(CNc4cccc5c4C(=O)N(C4CCC(=O)NC4=O)C5=O)c3)CC2)nc2ccncc21] |
| A68 | 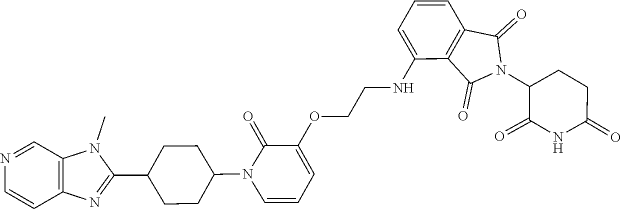 [SMILES: Cn1c(C2CCC(n3cccc(OCCNc4cccc5c4C(=O)N(C4CCC(=O)NC4=O)C5=O)c3=O)CC2)nc2ccncc21] |
| A69 | 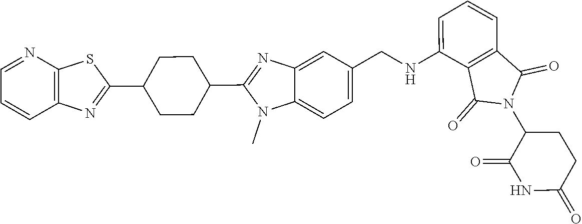 [SMILES: Cn1c(C2CCC(c3nc4cccnc4s3)CC2)nc2cc(CNc3cccc4c3C(=O)N(C3CCC(=O)NC3=O)C4=O)ccc21] |
| A70 | 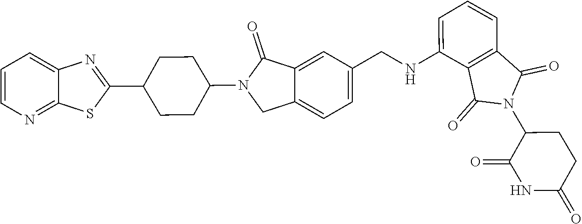 [SMILES: O=C1CCC(N2C(=O)c3cccc(NCc4ccc5c(c4)C(=O)N(C4CCC(c6nc7cccnc7s6)CC4)C5)c3C2=O)C(=O)N1] |
| A71 | 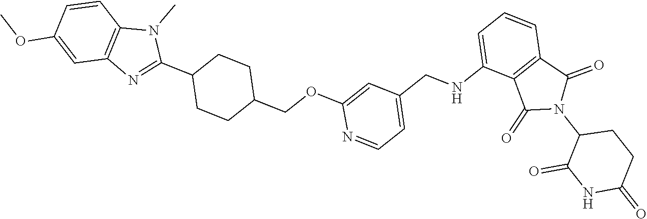 [SMILES: COc1ccc2c(c1)nc(C1CCC(COc3cc(CNc4cccc5c4C(=O)N(C4CCC(=O)NC4=O)C5=O)ccn3)CC1)n2C] |
| A72 | 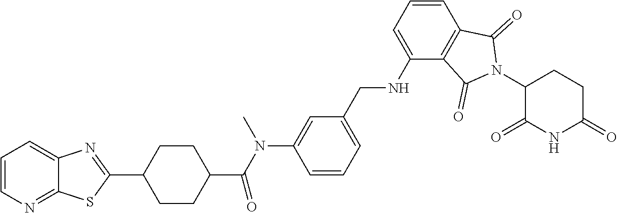 [SMILES: CN(C(=O)C1CCC(c2nc3cccnc3s2)CC1)c1cccc(CNc2cccc3c2C(=O)N(C2CCC(=O)NC2=O)C3=O)c1] |
| A73 | 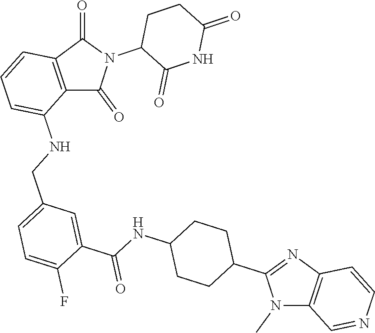 [SMILES: Cn1c(C2CCC(NC(=O)c3cc(CNc4cccc5c4C(=O)N(C4CCC(=O)NC4=O)C5=O)ccc3F)CC2)nc2ccncc21] |
| A74 | 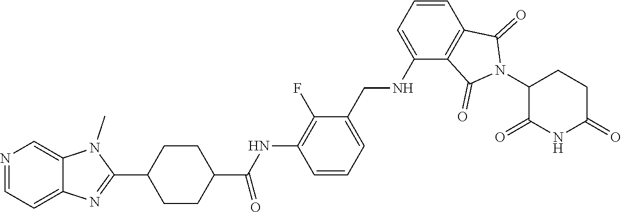 [SMILES: Cn1c(C2CCC(C(=O)Nc3cccc(CNc4cccc5c4C(=O)N(C4CCC(=O)NC4=O)C5=O)c3F)CC2)nc2ccncc21] |
| A75 | 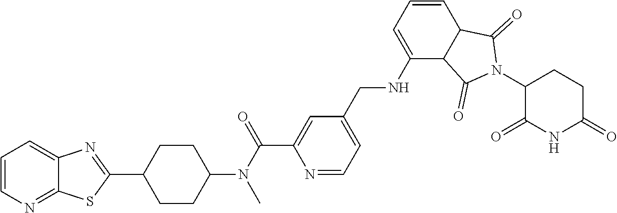 [SMILES: CN(C(=O)c1cc(CNC2=CC=CC3C(=O)N(C4CCC(=O)NC4=O)C(=O)C23)ccn1)C1CCC(c2nc3cccnc3s2)CC1] |
| A76 | 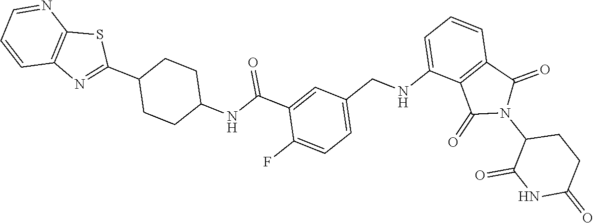 [SMILES: O=C1CCC(N2C(=O)c3cccc(NCc4ccc(F)c(C(=O)NC5CCC(c6nc7cccnc7s6)CC5)c4)c3C2=O)C(=O)N1] |
| A77 | 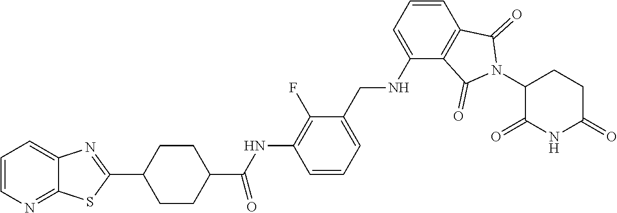 [SMILES: O=C1CCC(N2C(=O)c3cccc(NCc4cccc(NC(=O)C5CCC(c6nc7cccnc7s6)CC5)c4F)c3C2=O)C(=O)N1] |
| A78 | 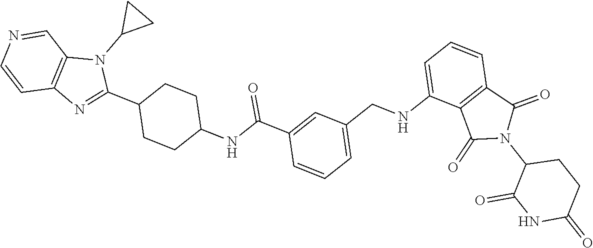 [SMILES: O=C1CCC(N2C(=O)c3cccc(NCc4cccc(C(=O)NC5CCC(c6nc7ccncc7n6C6CC6)CC5)c4)c3C2=O)C(=O)N1] |
| A79 | 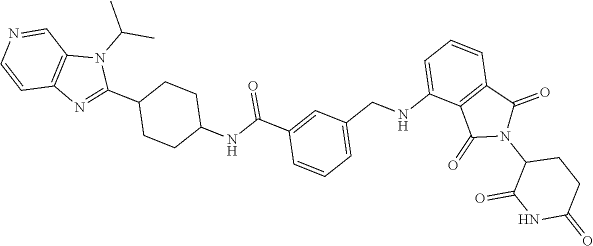 [SMILES: CC(C)n1c(C2CCC(NC(=O)c3cccc(CNc4cccc5c4C(=O)N(C4CCC(=O)NC4=O)C5=O)c3)CC2)nc2ccncc21] |
| A80 | 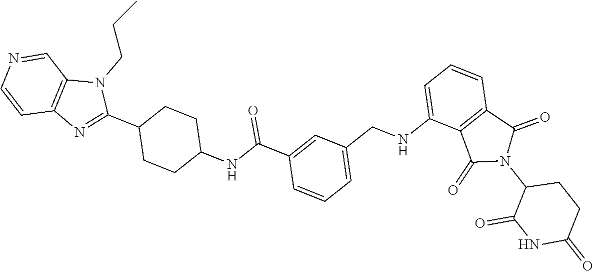 [SMILES: CCCn1c(C2CCC(NC(=O)c3cccc(CNc4cccc5c4C(=O)N(C4CCC(=O)NC4=O)C5=O)c3)CC2)nc2ccncc21] |
| A81 | 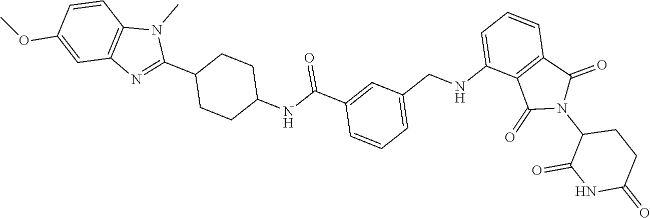 [SMILES: COc1ccc2c(c1)nc(C1CCC(NC(=O)c3cccc(CNc4cccc5c4C(=O)N(C4CCC(=O)NC4=O)C5=O)c3)CC1)n2C] |
| A82 | 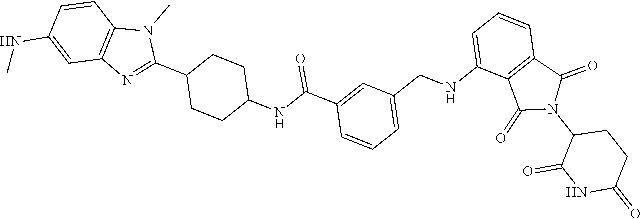 [SMILES: CNc1ccc2c(c1)nc(C1CCC(NC(=O)c3cccc(CNc4cccc5c4C(=O)N(C4CCC(=O)NC4=O)C5=O)c3)CC1)n2C] |
| A83 | 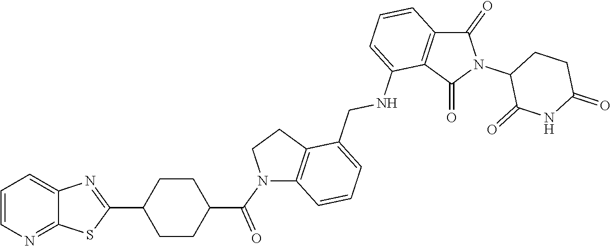 [SMILES: O=C1CCC(N2C(=O)c3cccc(NCc4cccc5c4CCN5C(=O)C4CCC(c5nc6cccnc6s5)CC4)c3C2=O)C(=O)N1] |
| A84 | 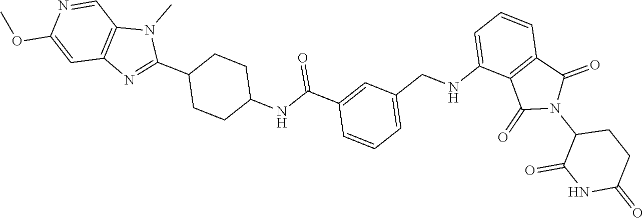 [SMILES: COc1cc2nc(C3CCC(NC(=O)c4cccc(CNc5cccc6c5C(=O)N(C5CCC(=O)NC5=O)C6=O)c4)CC3)n(C)c2cn1] |
| A85 | 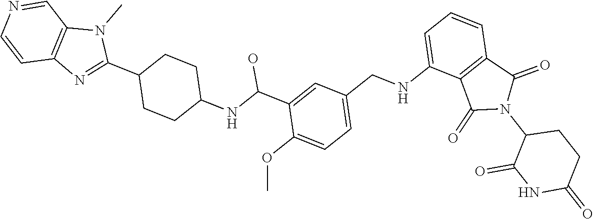 [SMILES: COc1ccc(CNc2cccc3c2C(=O)N(C2CCC(=O)NC2=O)C3=O)cc1C(O)NC1CCC(c2nc3ccncc3n2C)CC1] |
| A86 | 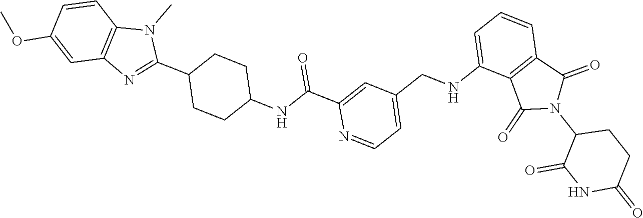 [SMILES: COc1ccc2c(c1)nc(C1CCC(NC(=O)c3cc(CNc4cccc5c4C(=O)N(C4CCC(=O)NC4=O)C5=O)ccn3)CC1)n2C] |
| A87 | 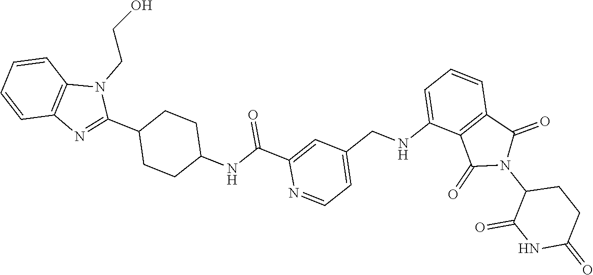 [SMILES: O=C1CCC(N2C(=O)c3cccc(NCc4ccnc(C(=O)NC5CCC(c6nc7ccccc7n6CCO)CC5)c4)c3C2=O)C(=O)N1] |
| A88 | 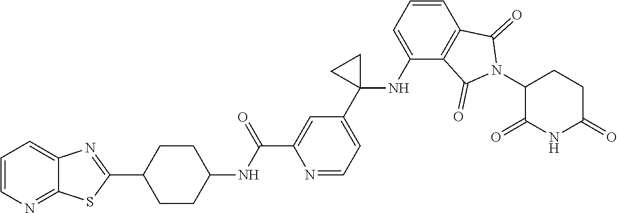 [SMILES: O=C1CCC(N2C(=O)c3cccc(NC4(c5ccnc(C(=O)NC6CCC(c7nc8cccnc8s7)CC6)c5)CC4)c3C2=O)C(=O)N1] |
| A89 | 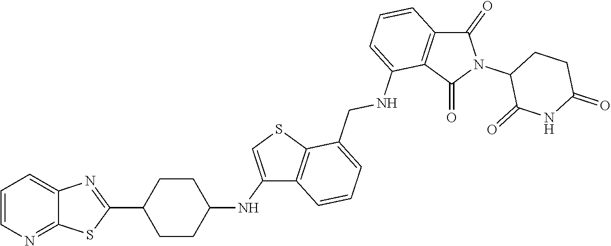 [SMILES: O=C1CCC(N2C(=O)c3cccc(NCc4cccc5c(NC6CCC(c7nc8cccnc8s7)CC6)csc45)c3C2=O)C(=O)N1] |
| A90 | 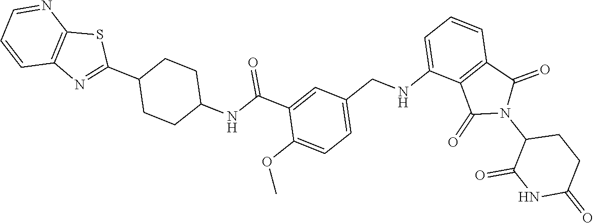 [SMILES: COc1ccc(CNc2cccc3c2C(=O)N(C2CCC(=O)NC2=O)C3=O)cc1C(=O)NC1CCC(c2nc3cccnc3s2)CC1] |
| A91 | 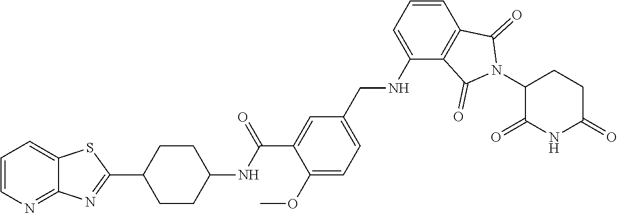 [SMILES: COc1ccc(CNc2cccc3c2C(=O)N(C2CCC(=O)NC2=O)C3=O)cc1C(=O)NC1CCC(c2nc3ncccc3s2)CC1] |
| A92 | 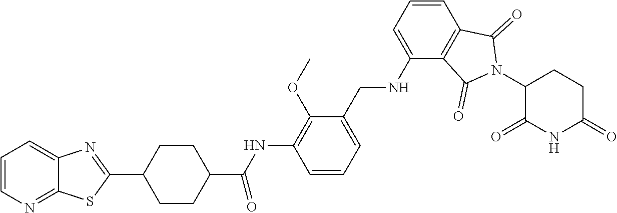 [SMILES: COc1c(CNc2cccc3c2C(=O)N(C2CCC(=O)NC2=O)C3=O)cccc1NC(=O)C1CCC(c2nc3cccnc3s2)CC1] |
| A93 | 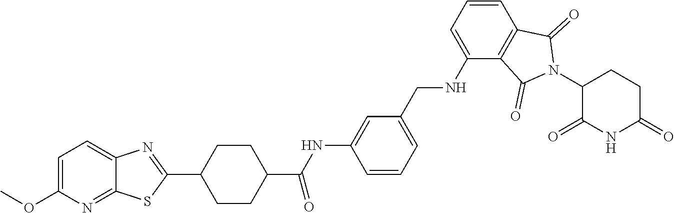 [SMILES: COc1ccc2nc(C3CCC(C(=O)Nc4cccc(CNc5cccc6c5C(=O)N(C5CCC(=O)NC5=O)C6=O)c4)CC3)sc2n1] |
| A94 | 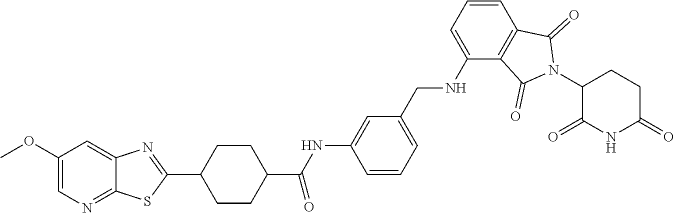 [SMILES: COc1cnc2sc(C3CCC(C(=O)Nc4cccc(CNc5cccc6c5C(=O)N(C5CCC(=O)NC5=O)C6=O)c4)CC3)nc2c1] |
| A95 | 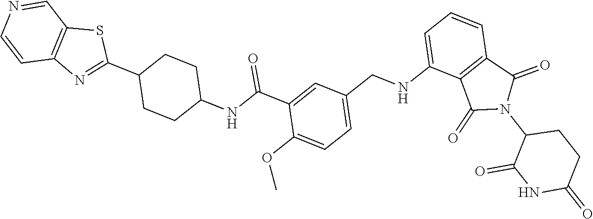 [SMILES: COc1ccc(CNc2cccc3c2C(=O)N(C2CCC(=O)NC2=O)C3=O)cc1C(=O)NC1CCC(c2nc3ccncc3s2)CC1] |
| A96 | 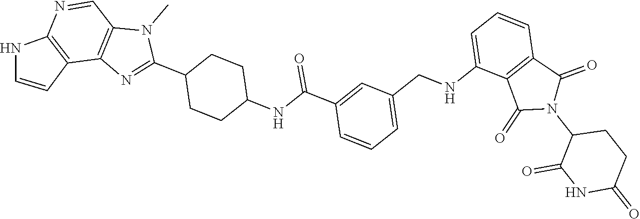 [SMILES: Cn1c(C2CCC(NC(=O)c3cccc(CNc4cccc5c4C(=O)N(C4CCC(=O)NC4=O)C5=O)c3)CC2)nc2c3cc[nH]c3ncc21] |
| A97 | 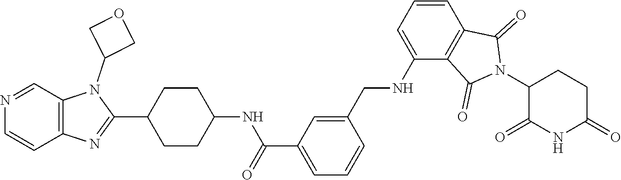 [SMILES: O=C1CCC(N2C(=O)c3cccc(NCc4cccc(C(=O)NC5CCC(c6nc7ccncc7n6C6COC6)CC5)c4)c3C2=O)C(=O)N1] |
| A98 | 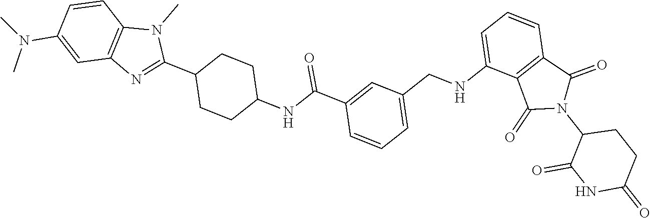 [SMILES: CN(C)c1ccc2c(c1)nc(C1CCC(NC(=O)c3cccc(CNc4cccc5c4C(=O)N(C4CCC(=O)NC4=O)C5=O)c3)CC1)n2C] |
| A99 | 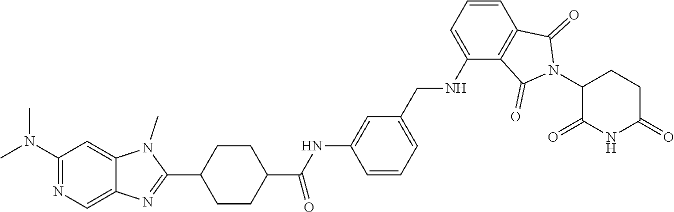 [SMILES: CN(C)c1cc2c(cn1)nc(C1CCC(C(=O)Nc3cccc(CNc4cccc5c4C(=O)N(C4CCC(=O)NC4=O)C5=O)c3)CC1)n2C] |
| A100 | 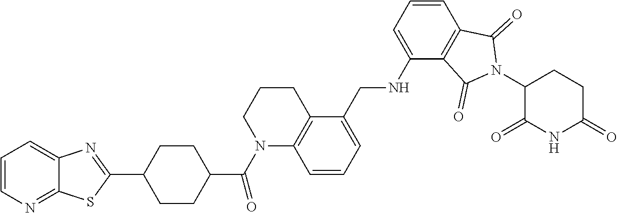 [SMILES: O=C1CCC(N2C(=O)c3cccc(NCc4cccc5c4CCCN5C(=O)C4CCC(c5nc6cccnc6s5)CC4)c3C2=O)C(=O)N1] |
| A101 | 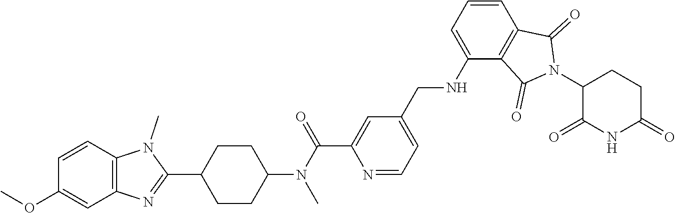 [SMILES: COc1ccc2c(c1)nc(C1CCC(N(C)C(=O)c3cc(CNc4cccc5c4C(=O)N(C4CCC(=O)NC4=O)C5=O)ccn3)CC1)n2C] |
| A102 | 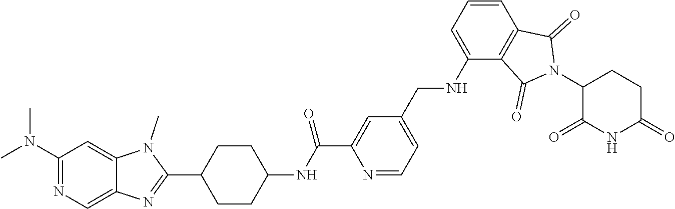 [SMILES: CN(C)c1cc2c(cn1)nc(C1CCC(NC(=O)c3cc(CNc4cccc5c4C(=O)N(C4CCC(=O)NC4=O)C5=O)ccn3)CC1)n2C] |
| A103 | 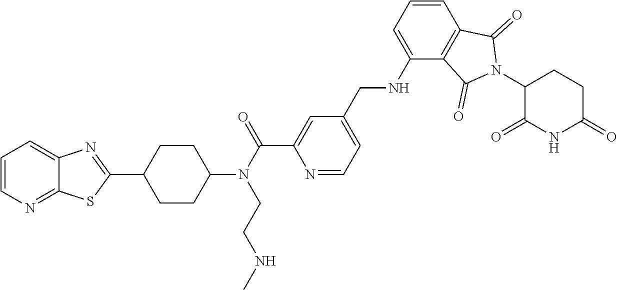 [SMILES: CNCCN(C(=O)c1cc(CNc2cccc3c2C(=O)N(C2CCC(=O)NC2=O)C3=O)ccn1)C1CCC(c2nc3cccnc3s2)CC1] |
| A104 | 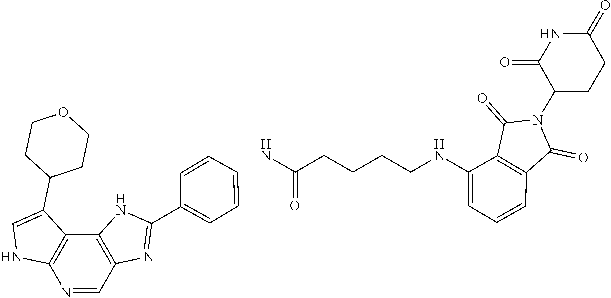 |
| A105 | 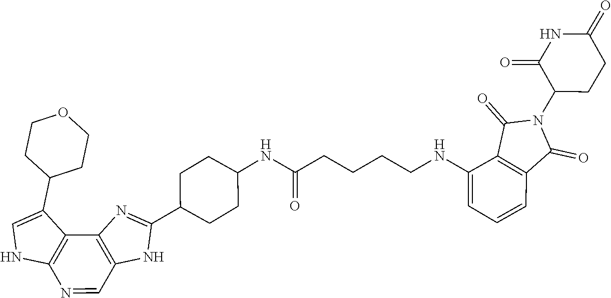 [SMILES: O=C1CCC(N2C(=O)c3cccc(NCCCCC(=O)NC4CCC(c5nc6c(cnc7[nH]cc(C8CCOCC8)c76)[nH]5)CC4)c3C2=O)C(=O)N1] |
| A106 | 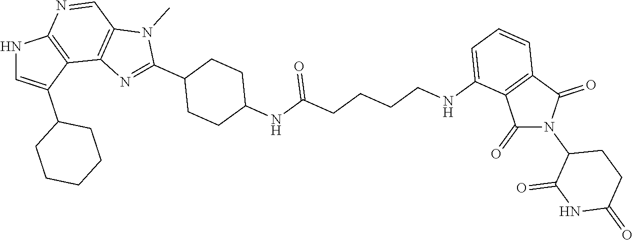 [SMILES: Cn1c(C2CCC(NC(=O)CCCCNc3cccc4c3C(=O)N(C3CCC(=O)NC3=O)C4=O)CC2)nc2c3c(C4CCCCC4)c[nH]c3ncc21] |
| A107 | 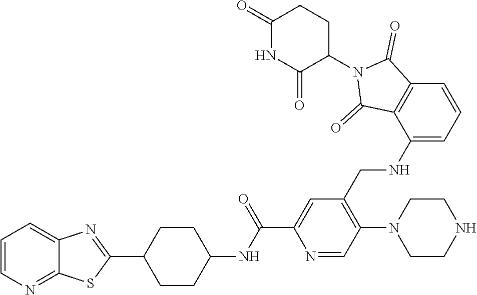 [SMILES: O=C1CCC(N2C(=O)c3cccc(NCc4cc(C(=O)NC5CCC(c6nc7cccnc7s6)CC5)ncc4N4CCNCC4)c3C2=O)C(=O)N1] |
| A108 | 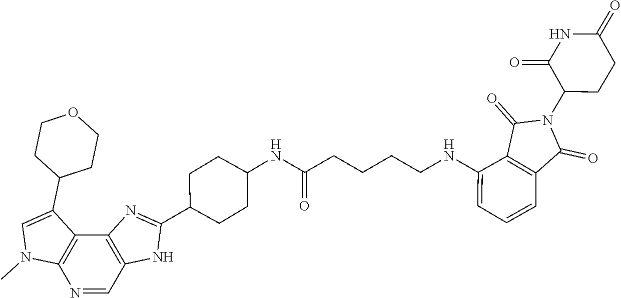 [SMILES: Cn1cc(C2CCOCC2)c2c3nc(C4CCC(NC(=O)CCCCNc5cccc6c5C(=O)N(C5CCC(=O)NC5=O)C6=O)CC4)[nH]c3cnc21] |
| A109 | 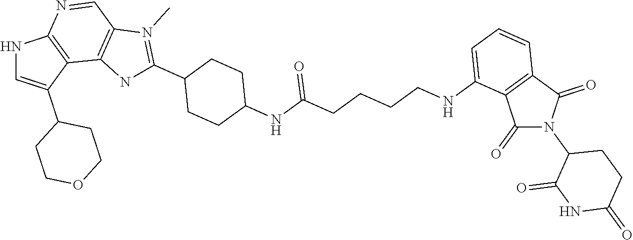 |
| A110 | 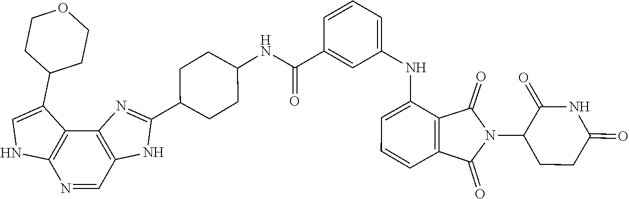 [SMILES: O=C1CCC(N2C(=O)c3cccc(Nc4cccc(C(=O)NC5CCC(c6nc7c(cnc8[nH]cc(C9CCOCC9)c87)[nH]6)CC5)c4)c3C2=O)C(=O)N1] |
| A111 | 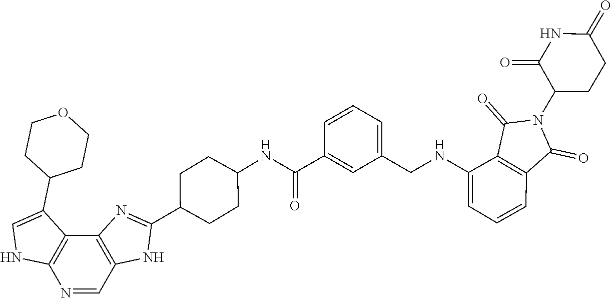 [SMILES: O=C1CCC(N2C(=O)c3cccc(NCc4cccc(C(=O)NC5CCC(c6nc7c(cnc8[nH]cc(C9CCOCC9)c87)[nH]6)CC5)c4)c3C2=O)C(=O)N1] |
| A112 | 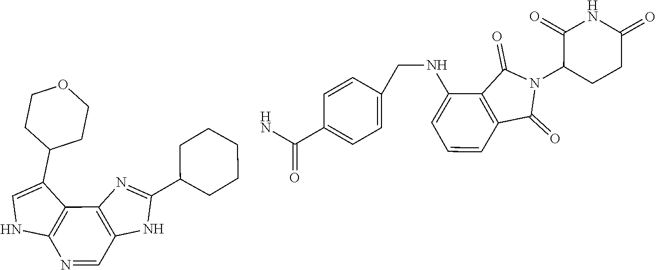 |
| A113 | 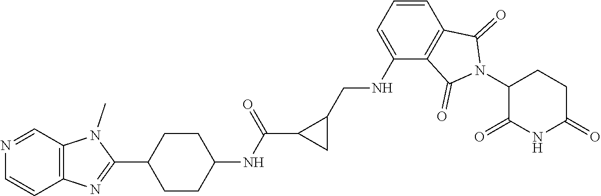 [SMILES: Cn1c(C2CCC(NC(=O)C3CC3CNc3cccc4c3C(=O)N(C3CCC(=O)NC3=O)C4=O)CC2)nc2ccncc21] |
| A114 | 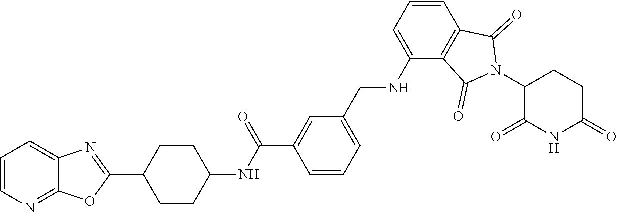 [SMILES: O=C1CCC(N2C(=O)c3cccc(NCc4cccc(C(=O)NC5CCC(c6nc7cccnc7o6)CC5)c4)c3C2=O)C(=O)N1] |
| A115 | 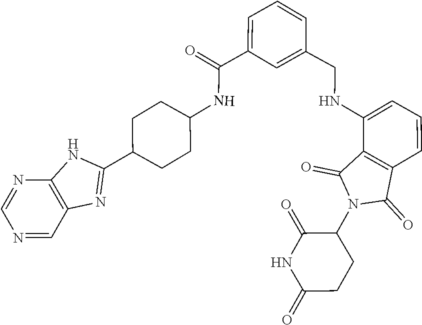 [SMILES: O=C1CCC(N2C(=O)c3cccc(NCc4cccc(C(=O)NC5CCC(c6nc7cncnc7[nH]6)CC5)c4)c3C2=O)C(=O)N1] |
| A116 | 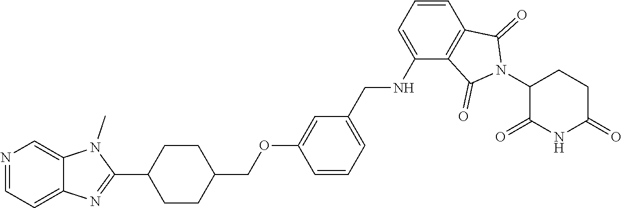 [SMILES: Cn1c(C2CCC(COc3cccc(CNc4cccc5c4C(=O)N(C4CCC(=O)NC4=O)C5=O)c3)CC2)nc2ccncc21] |
| A117 | 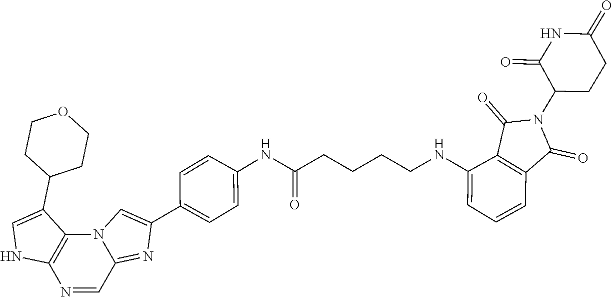 [SMILES: O=C1CCC(N2C(=O)c3cccc(NCCCCC(=O)Nc4ccc(-c5cn6c(cnc7[nH]cc(C8CCOCC8)c76)n5)cc4)c3C2=O)C(=O)N1] |
| A118 | 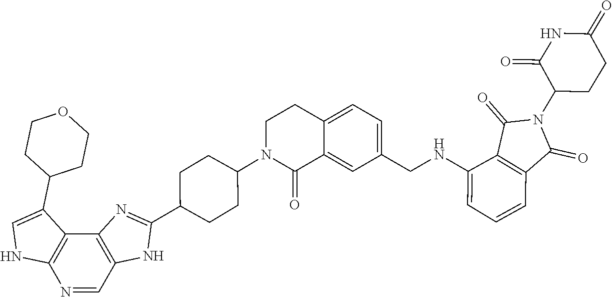 [SMILES: O=C1CCC(N2C(=O)c3cccc(NCc4ccc5c(c4)C(=O)N(C4CCC(c6nc7c(cnc8[nH]cc(C9CCOCC9)c87)[nH]6)CC4)CC5)c3C2=O)C(=O)N1] |
| A119 | 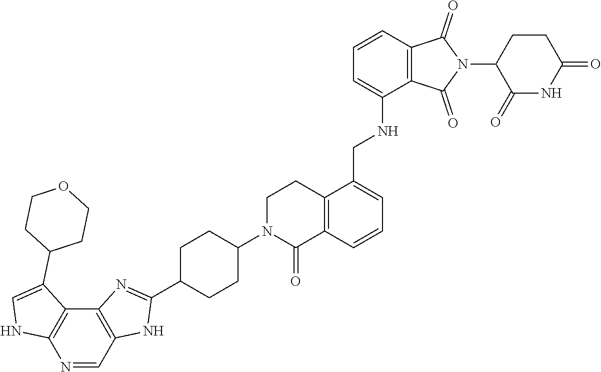 [SMILES: O=C1CCC(N2C(=O)c3cccc(NCc4cccc5c4CCN(C4CCC(c6nc7c(cnc8[nH]cc(C9CCOCC9)c87)[nH]6)CC4)C5=O)c3C2=O)C(=O)N1] |
| A120 | 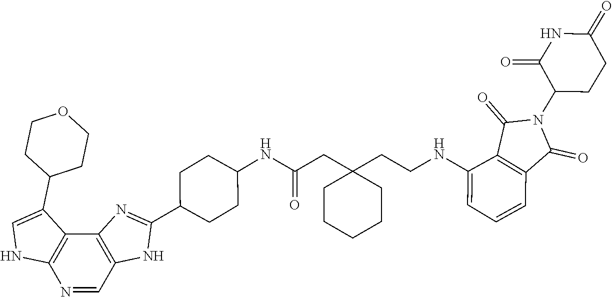 [SMILES: O=C1CCC(N2C(=O)c3cccc(NCCC4(CC(=O)NC5CCC(c6nc7c(cnc8[nH]cc(C9CCOCC9)c87)[nH]6)CC5)CCCCC4)c3C2=O)C(=O)N1] |
| A121 | 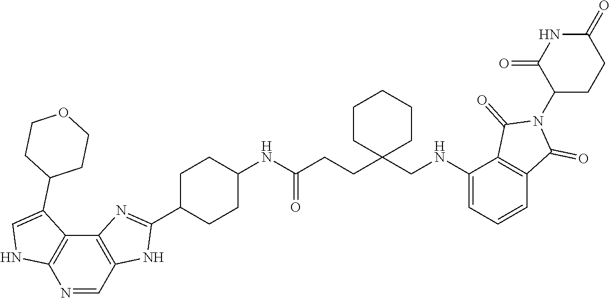 [SMILES: O=C1CCC(N2C(=O)c3cccc(NCC4(CCC(=O)NC5CCC(c6nc7c(cnc8[nH]cc(C9CCOCC9)c87)[nH]6)CC5)CCCCC4)c3C2=O)C(=O)N1] |
| A122 | 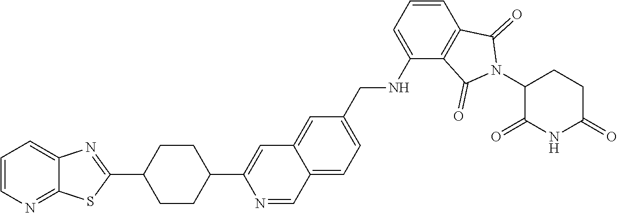 [SMILES: O=C1CCC(N2C(=O)c3cccc(NCc4ccc5cnc(C6CCC(c7nc8cccnc8s7)CC6)cc5c4)c3C2=O)C(=O)N1] |
| A123 | 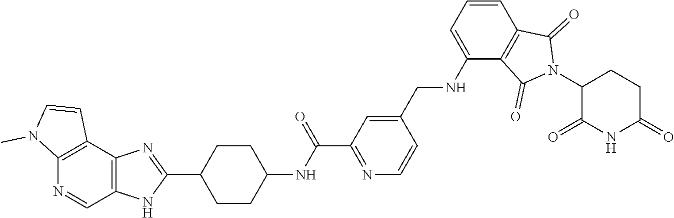 [SMILES: Cn1ccc2c3nc(C4CCC(NC(=O)c5cc(CNc6cccc7c6C(=O)N(C6CCC(=O)NC6=O)C7=O)ccn5)CC4)[nH]c3cnc21] |
| A124 | 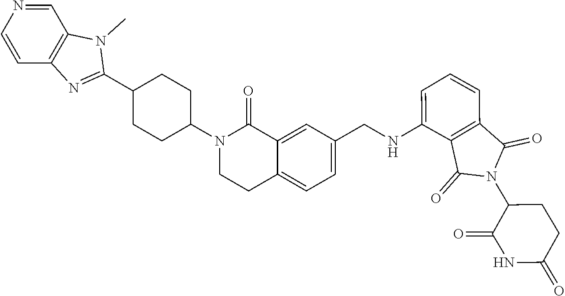 [SMILES: Cn1c(C2CCC(N3CCc4ccc(CNc5cccc6c5C(=O)N(C5CCC(=O)NC5=O)C6=O)cc4C3=O)CC2)nc2ccncc21] |
| A125 | 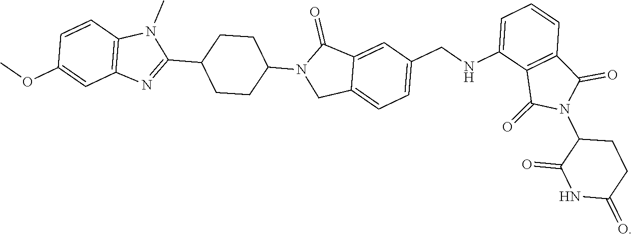 [SMILES: COc1ccc2c(c1)nc(C1CCC(N3Cc4ccc(CNc5cccc6c5C(=O)N(C5CCC(=O)NC5=O)C6=O)cc4C3=O)CC1)n2C] |

76. The compound according to claim 57, wherein the stereoisomer is selected from the following structures:

| ID | Structure |
|---|---|
| A21′ | 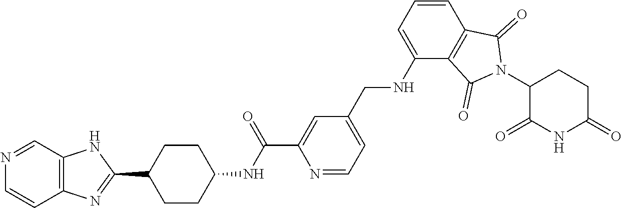 |
| A24′ | 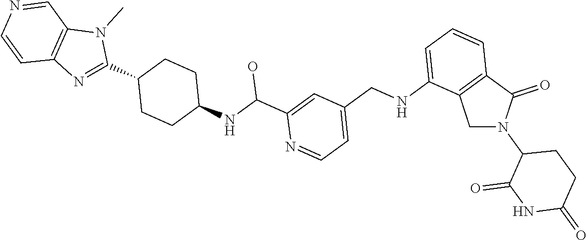 [SMILES: Cn1c2cnccc2nc1[C@H]1CC[C@H](NC(O)c2cc(CNc3cccc4c3CN(C3CCC(=O)NC3=O)C4=O)ccn2)CC1] |
| A26′ | 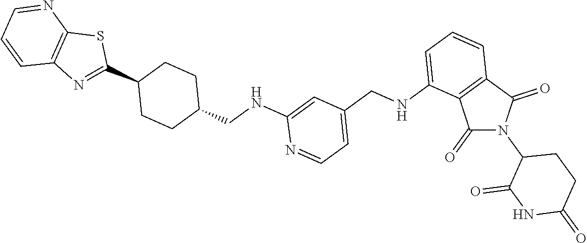 [SMILES: O=C1CCC(N2C(=O)c3cccc(NCc4ccnc(NC[C@H]5CC[C@H](c6nc7cccnc7s6)CC5)c4)c3C2=O)C(=O)N1] |
| A28′ | 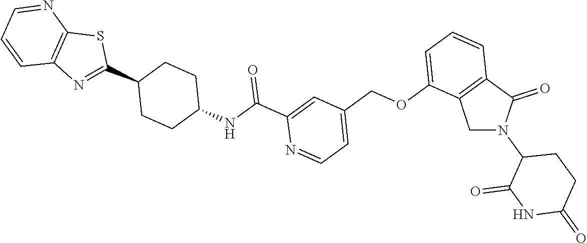 [SMILES: O=C1CCC(N2Cc3c(OCc4ccnc(C(=O)N[C@H]5CC[C@H](c6nc7cccnc7s6)CC5)c4)cccc3C2=O)C(=O)N1] |
| A29′ | 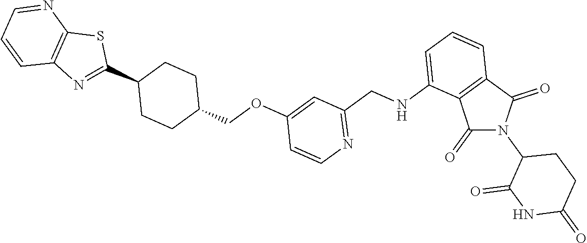 [SMILES: O=C1CCC(N2C(=O)c3cccc(NCc4cc(OC[C@H]5CC[C@H](c6nc7cccnc7s6)CC5)ccn4)c3C2=O)C(=O)N1] |
| A32′ | 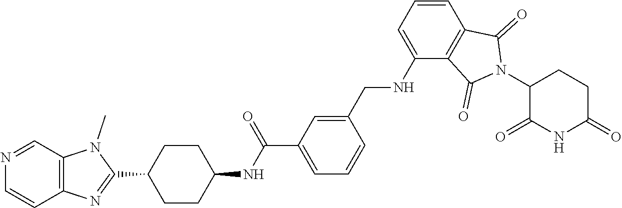 [SMILES: Cn1c2cnccc2nc1[C@H]1CC[C@H](NC(=O)c2cccc(CNc3cccc4c3C(=O)N(C3CCC(=O)NC3=O)C4=O)c2)CC1] |
| A33′ | 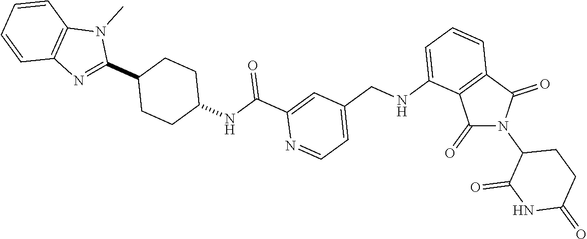 |
| A34′ | 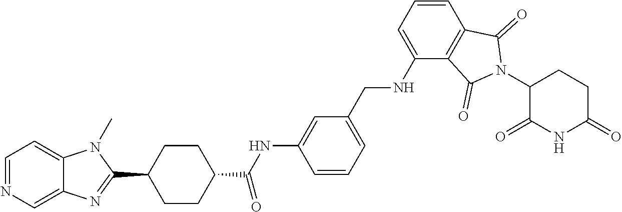 [SMILES: Cn1c2ccncc2nc1[C@H]1CC[C@H](C(=O)Nc2cccc(CNc3cccc4c3C(=O)N(C3CCC(=O)NC3=O)C4=O)c2)CC1] |
| A35′ | 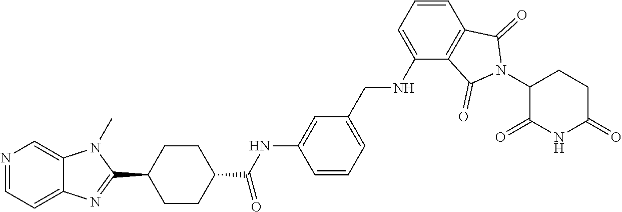 [SMILES: Cn1c2cnccc2nc1[C@H]1CC[C@H](C(=O)Nc2cccc(CNc3cccc4c3C(=O)N(C3CCC(=O)NC3=O)C4=O)c2)CC1] |
| A36′ | 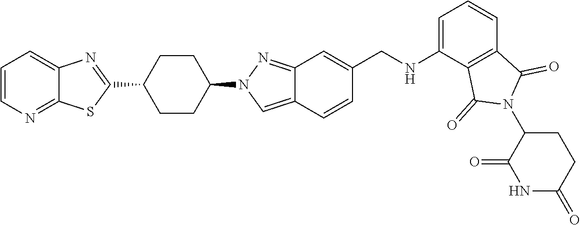 [SMILES: O=C1CCC(N2C(=O)c3cccc(NCc4ccc5cn([C@H]6CC[C@H](c7nc8cccnc8s7)CC6)nc5c4)c3C2=O)C(=O)N1] |
| A37′ | 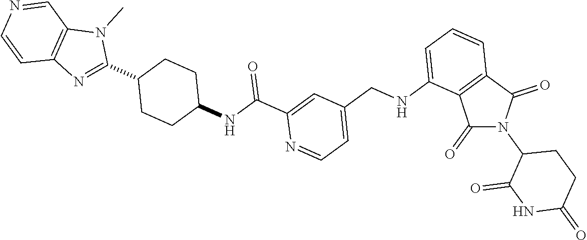 |
| A40′ | 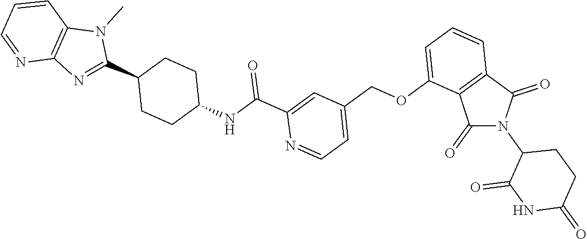 [SMILES: Cn1c2cccnc2nc1[C@H]1CC[C@H](NC(=O)c2cc(COc3cccc4c3C(=O)N(C3CCC(=O)NC3=O)C4=O)ccn2)CC1] |
| A41′ | 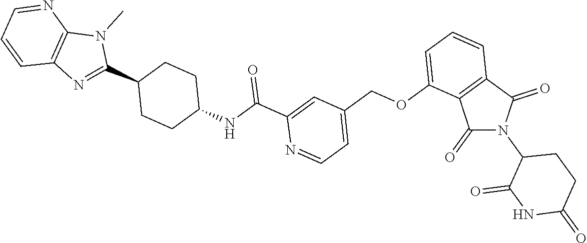 [SMILES: Cn1c2ncccc2nc1[C@H]1CC[C@H](NC(=O)c2cc(COc3cccc4c3C(=O)N(C3CCC(=O)NC3=O)C4=O)ccn2)CC1] |
| A43′ | 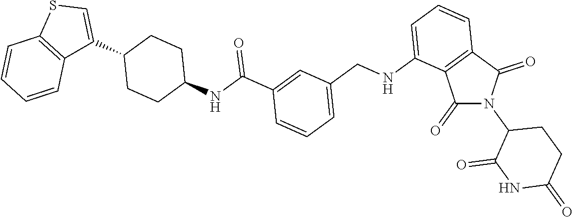 [SMILES: O=C1CCC(N2C(=O)c3cccc(NCc4cccc(C(=O)N[C@H]5CC[C@H](c6csc7ccccc76)CC5)c4)c3C2=O)C(=O)N1] |
| A44′ | 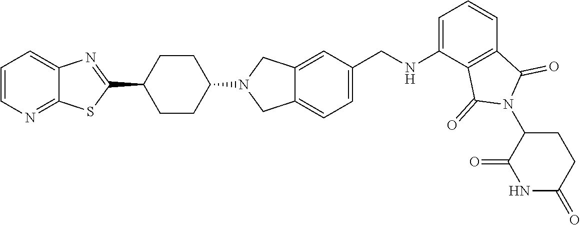 [SMILES: O=C1CCC(N2C(=O)c3cccc(NCc4ccc5c(c4)CN([C@H]4CC[C@H](c6nc7cccnc7s6)CC4)C5)c3C2=O)C(=O)N1] |
| A55′ | 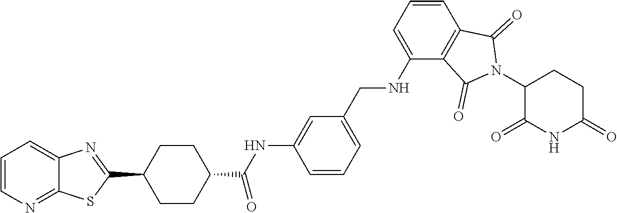 [SMILES: O=C1CCC(N2C(=O)c3cccc(NCc4cccc(NC(=O)[C@H]5CC[C@H](c6nc7cccnc7s6)CC5)c4)c3C2=O)C(=O)N1] |
| A56′ | 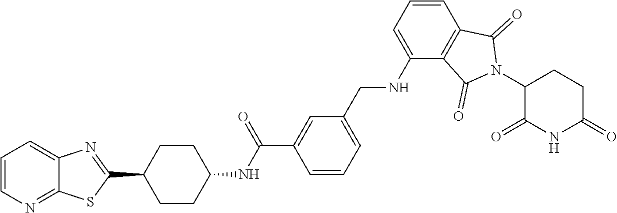 [SMILES: O=C1CCC(N2C(=O)c3cccc(NCc4cccc(C(=O)N[C@H]5CC[C@H](c6nc7cccnc7s6)CC5)c4)c3C2=O)C(=O)N1] |
| A58′ | 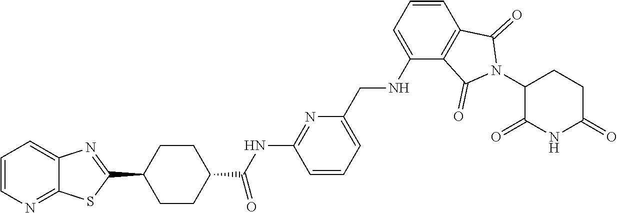 [SMILES: O=C1CCC(N2C(=O)c3cccc(NCc4cccc(NC(=O)[C@H]5CC[C@H](c6nc7cccnc7s6)CC5)n4)c3C2=O)C(=O)N1] |
| A59′ | 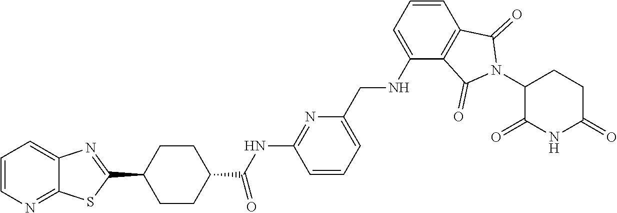 [SMILES: O=C1CCC(N2C(=O)c3cccc(NCc4cccc(NC(=O)[C@H]5CC[C@H](c6nc7cccnc7s6)CC5)n4)c3C2=O)C(=O)N1] |
| A60′ | 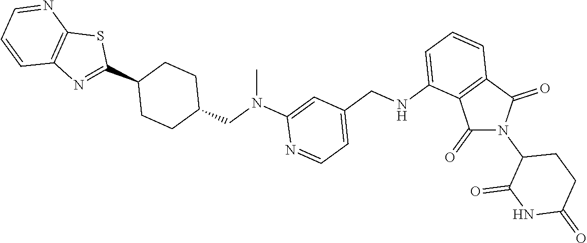 [SMILES: CN(C[C@H]1CC[C@H](c2nc3cccnc3s2)CC1)c1cc(CNc2cccc3c2C(=O)N(C2CCC(=O)NC2=O)C3=O)ccn1] |
| A64′ | 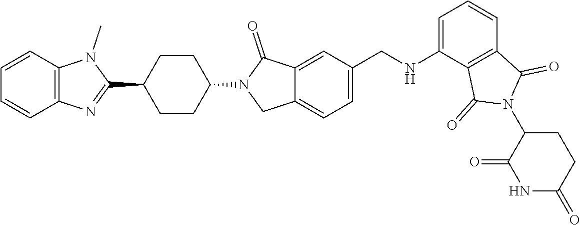 [SMILES: Cn1c2ccccc2nc1[C@H]1CC[C@H](N2Cc3ccc(CNc4cccc5c4C(=O)N(C4CCC(=O)NC4=O)C5=O)cc3C2=O)CC1] |
| A66′ | 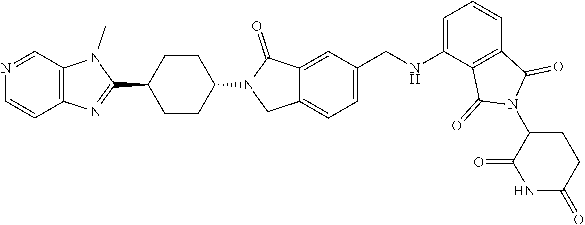 [SMILES: Cn1c2cnccc2nc1[C@H]1CC[C@H](N2Cc3ccc(CNc4cccc5c4C(=O)N(C4CCC(=O)NC4=O)C5=O)cc3C2=O)CC1] |
| A69′ | 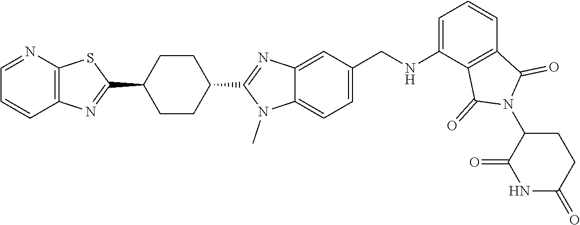 [SMILES: Cn1c2ccc(CNc3cccc4c3C(=O)N(C3CCC(=O)NC3=O)C4=O)cc2nc1[C@H]1CC[C@H](c2nc3cccnc3s2)CC1] |
| A70′ | 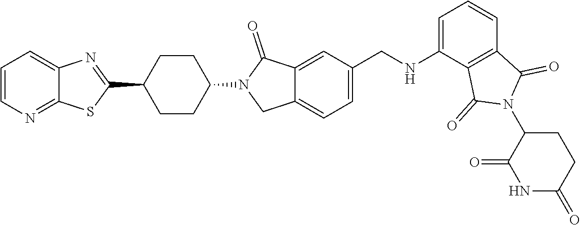 [SMILES: O=C1CCC(N2C(=O)c3cccc(NCc4ccc5c(c4)C(=O)N([C@H]4CC[C@H](c6nc7cccnc7s6)CC4)C5)c3C2=O)C(=O)N1] |
| A71′ | 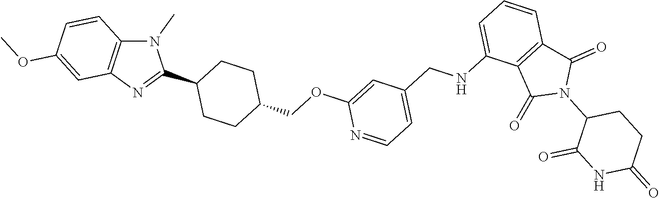 [SMILES: COc1ccc2c(c1)nc([C@H]1CC[C@H](COc3cc(CNc4cccc5c4C(=O)N(C4CCC(=O)NC4=O)C5=O)ccn3)CC1)n2C] |
| A72′ | 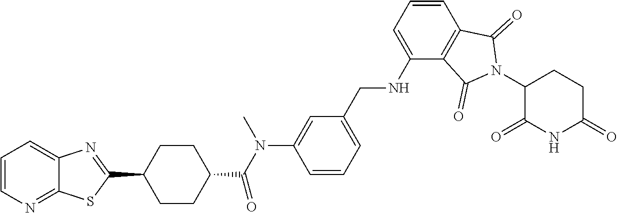 [SMILES: CN(c1cccc(CNc2cccc3c2C(=O)N(C2CCC(=O)NC2=O)C3=O)c1)C(=O)[C@H]1CC[C@H](c2nc3cccnc3s2)CC1] |
| A74′ | 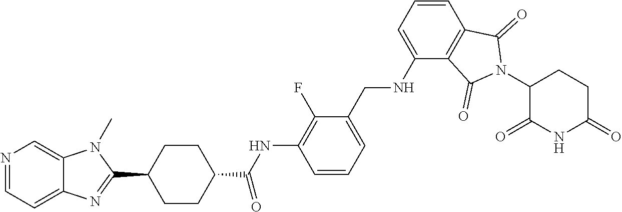 [SMILES: Cn1c2cnccc2nc1[C@H]1CC[C@H](C(=O)Nc2cccc(CNc3cccc4c3C(=O)N(C3CCC(=O)NC3=O)C4=O)c2F)CC1] |
| A75′ | 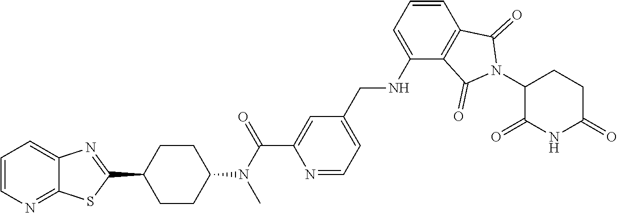 [SMILES: CN(C(=O)c1cc(CNc2cccc3c2C(=O)N(C2CCC(=O)NC2=O)C3=O)ccn1)[C@H]1CC[C@H](c2nc3cccnc3s2)CC1] |
| A76′ | 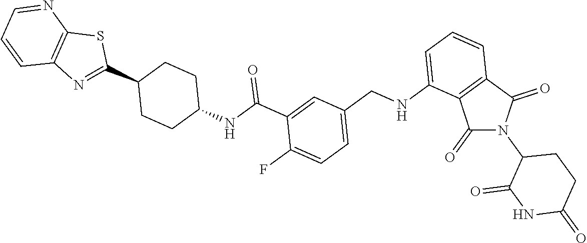 [SMILES: O=C1CCC(N2C(=O)c3cccc(NCc4ccc(F)c(C(=O)N[C@H]5CC[C@H](c6nc7cccnc7s6)CC5)c4)c3C2=O)C(=O)N1] |
| A77′ | 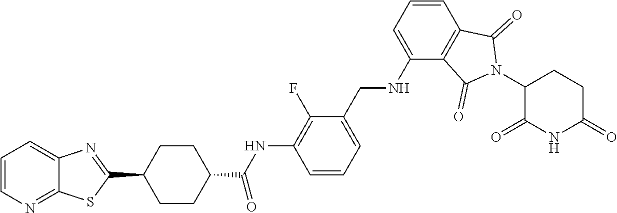 [SMILES: O=C1CCC(N2C(=O)c3cccc(NCc4cccc(NC(=O)[C@H]5CC[C@H](c6nc7cccnc7s6)CC5)c4F)c3C2=O)C(=O)N1] |
| A83′ | 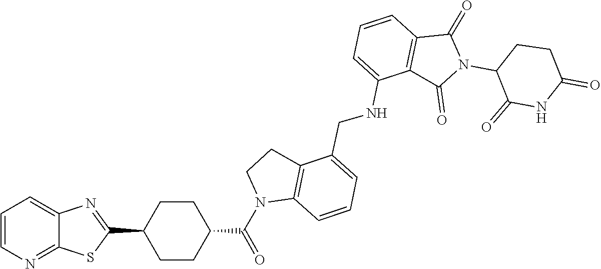 [SMILES: O=C1CCC(N2C(=O)c3cccc(NCc4cccc5c4CCN5C(=O)[C@H]4CC[C@H](c5nc6cccnc6s5)CC4)c3C2=O)C(=O)N1] |
| A85′ | 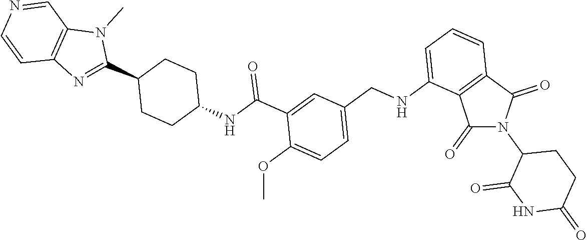 [SMILES: COc1ccc(CNc2cccc3c2C(=O)N(C2CCC(=O)NC2=O)C3=O)cc1C(=O)N[C@H]1CC[C@H](c2nc3ccncc3n2C)CC1] |
| A86′ | 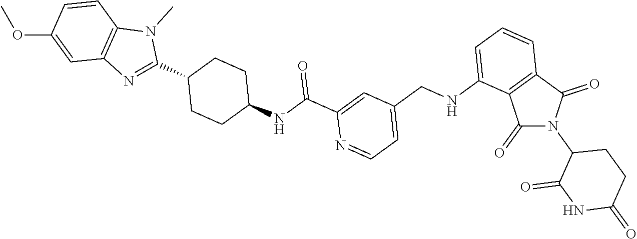 [SMILES: COc1ccc2c(c1)nc([C@H]1CC[C@H](NC(=O)c3cc(CNc4cccc5c4C(=O)N(C4CCC(=O)NC4=O)C5=O)ccn3)CC1)n2C] |
| A87′ | 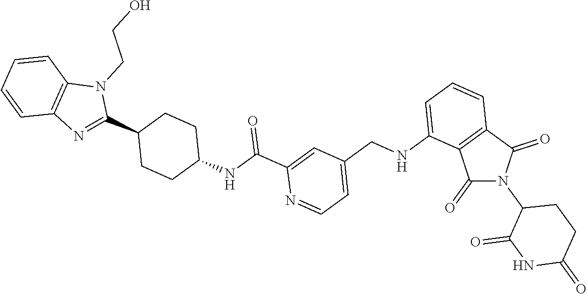 [SMILES: O=C1CCC(N2C(=O)c3cccc(NCc4ccnc(C(=O)N[C@H]5CC[C@H](c6nc7ccccc7n6CCO)CC5)c4)c3C2=O)C(=O)N1] |
| A88′ | 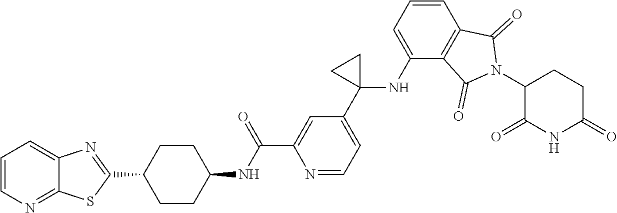 [SMILES: O=C1CCC(N2C(=O)c3cccc(NC4(c5ccnc(C(=O)N[C@H]6CC[C@H](c7nc8cccnc8s7)CC6)c5)CC4)c3C2=O)C(=O)N1] |
| A89′ | 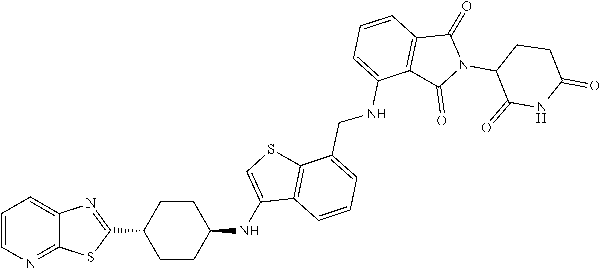 [SMILES: O=C1CCC(N2C(=O)c3cccc(NCc4cccc5c(N[C@H]6CC[C@H](c7nc8cccnc8s7)CC6)csc45)c3C2=O)C(=O)N1] |
| A90′ | 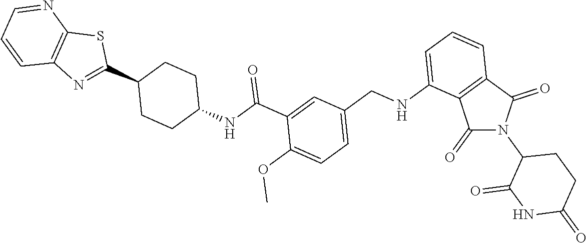 [SMILES: COc1ccc(CNc2cccc3c2C(=O)N(C2CCC(=O)NC2=O)C3=O)cc1C(=O)N[C@H]1CC[C@H](c2nc3cccnc3s2)CC1] |
| A91′ | 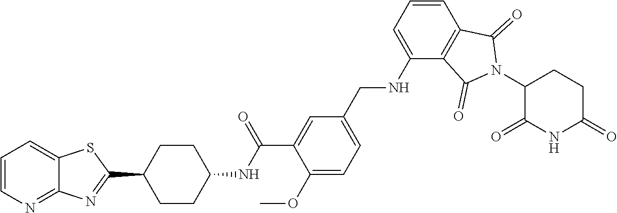 [SMILES: COc1ccc(CNc2cccc3c2C(=O)N(C2CCC(=O)NC2=O)C3=O)cc1C(=O)N[C@H]1CC[C@H](c2nc3ncccc3s2)CC1] |
| A92′ | 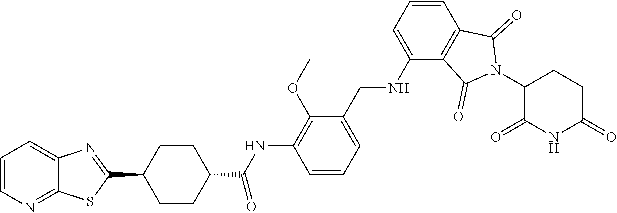 [SMILES: COc1c(CNc2cccc3c2C(=O)N(C2CCC(=O)NC2=O)C3=O)cccc1NC(=O)[C@H]1CC[C@H](c2nc3cccnc3s2)CC1] |
| A93′ | 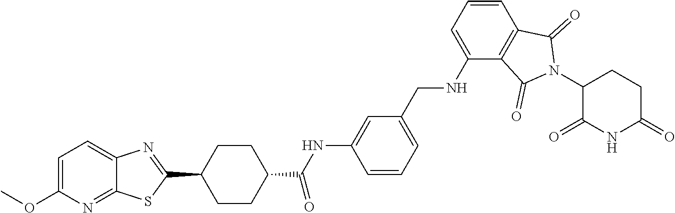 [SMILES: COc1ccc2nc([C@H]3CC[C@H](C(=O)Nc4cccc(CNc5cccc6c5C(=O)N(C5CCC(=O)NC5=O)C6=O)c4)CC3)sc2n1] |
| A94′ | 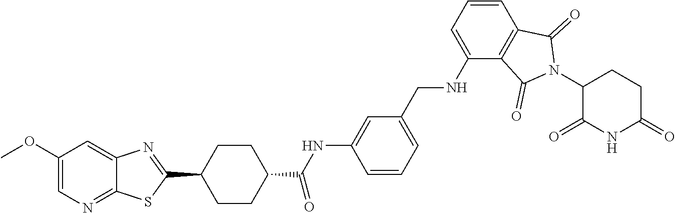 [SMILES: COc1cnc2sc([C@H]3CC[C@H](C(=O)Nc4cccc(CNc5cccc6c5C(=O)N(C5CCC(=O)NC5=O)C6=O)c4)CC3)nc2c1] |
| A95′ | 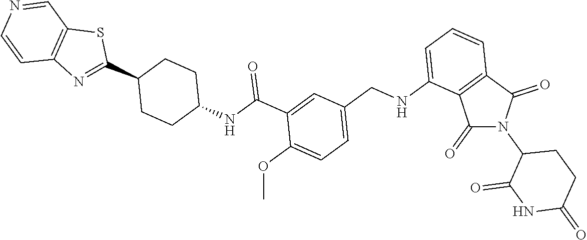 [SMILES: COc1ccc(CNc2cccc3c2C(=O)N(C2CCC(=O)NC2=O)C3=O)cc1C(=O)N[C@H]1CC[C@H](c2nc3ccncc3s2)CC1] |
| A99′ | 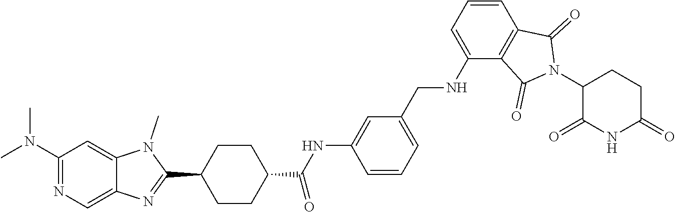 [SMILES: CN(C)c1cc2c(cn1)nc([C@H]1CC[C@H](C(=O)Nc3cccc(CNc4cccc5c4C(=O)N(C4CCC(=O)NC4=O)C5=O)c3)CC1)n2C] |
| A100′ | 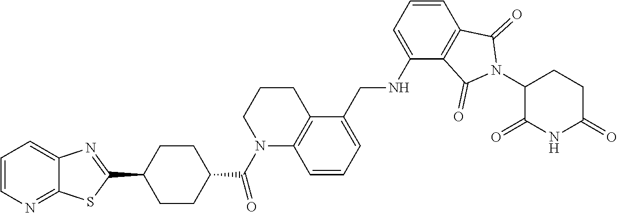 [SMILES: O=C1CCC(N2C(=O)c3cccc(NCc4cccc5c4CCCN5C(=O)[C@H]4CC[C@H](c5nc6cccnc6s5)CC4)c3C2=O)C(=O)N1] |
| A101′ | 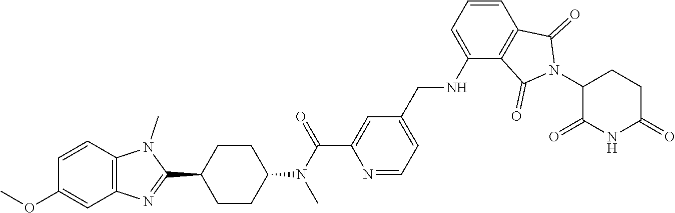 [SMILES: COc1ccc2c(c1)nc([C@H]1CC[C@H](N(C)C(=O)c3cc(CNc4cccc5c4C(=O)N(C4CCC(=O)NC4=O)C5=O)ccn3)CC1)n2C] |
| A102′ | 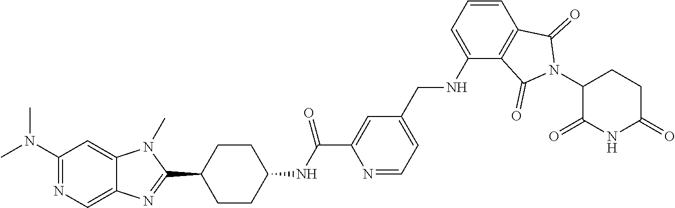 [SMILES: CN(C)c1cc2c(cn1)nc([C@H]1CC[C@H](NC(=O)c3cc(CNc4cccc5c4C(=O)N(C4CCC(=O)NC4=O)C5=O)ccn3)CC1)n2C] |
| A103′ | 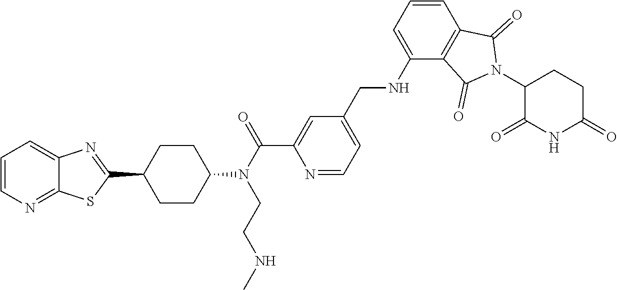 [SMILES: CNCCN(C(=O)c1cc(CNc2cccc3c2C(=O)N(C2CCC(=O)NC2=O)C3=O)ccn1)[C@H]1CC[C@H](c2nc3cccnc3s2)CC1] |
| A107′ | 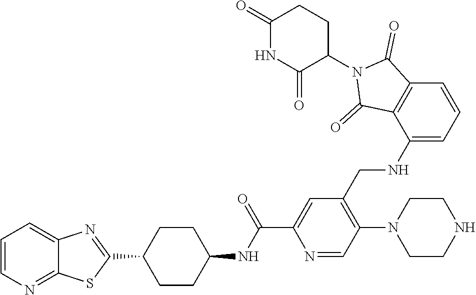 [SMILES: O=C1CCC(N2C(=O)c3cccc(NCc4cc(C(=O)N[C@H]5CC[C@H](c6nc7cccnc7s6)CC5)ncc4N4CCNCC4)c3C2=O)C(=O)N1] |
| A116′ | 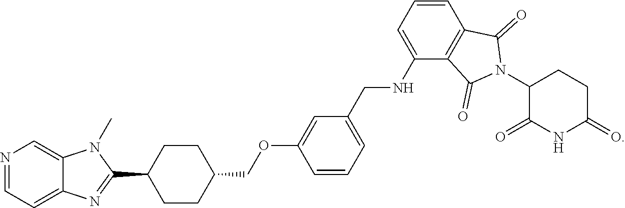 [SMILES: Cn1c2cnccc2nc1[C@H]1CC[C@H](COc2cccc(CNc3cccc4c3C(=O)N(C3CCC(=O)NC3=O)C4=O)c2)CC1] |

77. A pharmaceutical composition comprising the compound according to any one of claims 57 to 76, or a pharmaceutically acceptable salt, a stereoisomer, an ester, a prodrug, a solvate and a deuterated compound thereof.

78. Use of the compound according to any one of claims 57 to 76, or a pharmaceutically acceptable salt, a stereoisomer, an ester, a prodrug, a solvate and a deuterated compound thereof, in preparing a medicament for preventing and/or treating a GSPT1-associated disease.

79. The use according to claim 78, wherein the GSPT1-associated disease is selected from: an autoimmune disease, an inflammatory disease, a heteroimmune disease, a neurodegenerative disease, and a tumor;
preferably, the autoimmune disease is selected from: one or more of organ-specific autoimmune diseases, systemic lupus erythematosus, rheumatoid arthritis, systemic vasculitis, scleroderma, pemphigus, dermatomyositis, mixed connective tissue disease, autoimmune hemolytic anemia, thyroid autoimmune disease and ulcerative colitis;
preferably, the inflammatory disease is selected from: one or more of osteoarthritis, gout, chronic obstructive pulmonary disease, periodic fever, rash, lymphadenectasis, sepsis, osteoarthritis, ankylosing spondylitis, psoriasis, inflammatory bowel disease, Crohn's disease, ulcerative colitis, uveitis, asthma, and allergy;
preferably, the neurodegenerative disease is selected from: Alzheimer's disease, Parkinson's disease, Huntington's disease, amyotrophic lateral sclerosis, different types of spinocerebellar ataxia, and Pick's disease;
preferably, the tumor is selected from: adrenal cancer, anal cancer, angiosarcoma, appendiceal cancer, biliary tract cancer, bladder cancer, breast cancer, brain cancer, bronchial cancer, carcinoid tumor, cervical cancer, choriocarcinoma, chordoma, craniopharyngioma, colorectal cancer, connective tissue cancer, epithelial cancer, ependymoma, endothelial sarcoma, endometrial cancer, esophageal cancer, Ewing sarcoma, eye cancer, gallbladder cancer, gastric cancer, gastrointestinal stromal tumor (GIST), germ cell cancer, head and neck cancer, hematologic malignancies, hemangioblastoma, hypopharynx cancer, inflammatory myofibroblastoma, immune cell amyloidosis, kidney cancer, liver cancer, lung cancer, leiomyosarcoma (LMS), muscle cancer, mesothelioma, myeloproliferative disease (MPD), neuroblastoma, neurofibroma, neuroendocrine cancer, osteosarcoma, ovarian cancer, papillary adenocarcinoma, pancreatic cancer, penile cancer, pineal tumor, primitive neuroectodermal tumor (PNT), prostate cancer, rhabdomyosarcoma, salivary gland cancer, skin cancer, small bowel cancer, soft tissue sarcoma, sebaceous gland cancer, sweat gland cancer, synovial tumor, testicular cancer, thyroid cancer, urethral cancer, vaginal cancer, and vulval cancer.

80. The use according to claim 78, wherein the GSPT1-associated disease is a hematologic malignancy;
preferably, the hematologic malignancy is selected from: leukemia, lymphoma, and multiple myeloma;
preferably, the leukemia is chronic lymphocytic leukemia, chronic myelogenous leukemia, acute lymphocytic leukemia, acute myelogenous leukemia, or acute monocytic leukemia, particularly acute myelogenous leukemia;
preferably, the lymphoma is B-cell lymphoma, T-cell lymphoma or NK/T-cell lymphoma, particularly diffuse large B-cell lymphoma.
